(19) United States
(12) Patent Application Publication
(10) Pub. No.: US 2024/0398989 A1
(43) Pub. Date: Dec. 5, 2024

(54) METHODS AND COMPOSITIONS FOR DIAGNOSIS AND TREATMENT OF METABOLIC DISORDERS

(71) Applicants: The Broad Institute, Inc., Cambridge, MA (US); Beth Israel Deaconess Medical Center, Boston, MA (US)

(72) Inventors: Melina Claussnitzer, Boston, MA (US); Hesamaddin Dashti, Cambridge, MA (US); Samantha Laber, Cambridge, MA (US); Sophie Strobel, Cambridge, MA (US)

(21) Appl. No.: 18/576,444

(22) PCT Filed: Jul. 6, 2022
(86) PCT No.: PCT/US22/73454

§ 371 Date: Jan. 4, 2024

Related U.S. Application Data
(60) Provisional application No. 63/218,656, filed on Jul. 6, 2021.

(51) Int. Cl. A61K 48/00; A61K 35/35; A61K 38/17; C12Q 1/6883; G01N 33/50

(52) U.S. Cl. CPC A61K 48/0058; A61K 35/35; A61K 38/1709; C12Q 1/6883; G01N 33/5044; C12Q 2600/136; C12Q 2600/156

(57) ABSTRACT

Most disease-associated genetic loci map to more than one disease or trait, suggesting they act through multiple cell types and tissues giving rise to complex disease phenotypes. This pervasive pleiotropy of human diseases presents a tremendous burden on identifying mediating mechanisms and therapeutic targets. Multiple metabolic risk haplotypes are associated with risk for metabolic diseases. However, whether a haplotype actually causes a disease and the mechanisms that cause the disease are unknown. Integration of phenotypic and transcriptional profiling in primary human cells allows for functional characterization of disease-associated genetic variants. Applicants have analyzed multiple risk haplotypes and determined the function of risk haplotypes involved in causation of specific metabolic phenotypes, such as type 2 diabetes and lipodystrophy. Methods of treatments are disclosed herein.

### CROSS-REFERENCE TO RELATED APPLICATIONS

This application claims the benefit of U.S. Provisional Application No. 63/218,656, filed Jul. 6, 2021. The entire contents of the above-identified application are hereby fully incorporated herein by reference.

### REFERENCE TO AN ELECTRONIC SEQUENCE LISTING

The contents of the electronic sequence listing (“BROD-5380WP.xml”; Size is 89,008 bytes and it was created on Jul. 6, 2022) is herein incorporated by reference in its entirety.

### TECHNICAL FIELD

The subject matter disclosed herein is generally directed to compositions and methods for increasing COBLL1 expression or activity in adipocytes or BCL2 expression or activity to treat cardio-metabolic diseases, such as type 2 diabetes. The subject matter disclosed herein is also generally directed to adipocyte morphological and cellular profiling and metabolic genetic and polygenic risk.

### BACKGROUND

Obesity and type 2 diabetes related traits are intimately linked by both environmental and genetic factors. The global prevalence of obesity and type 2 diabetes (T2D) has risen dramatically over the past century, and both diseases constitute a severely increasing health problem worldwide, with T2D) being predicted to rise in prevalence from 451 to 693 million people between the years 2017 and 2045 (Cho et al. 2018). Most epidemiological and genetic studies have linked obesity to the pathogenesis of T2D through positive phenotypic correlations between adiposity and T2D. However, a small number of loci have been reported that do not follow this observation or even correlate in the opposite phenotypic direction (Lu et al. 2016). In fact, up to 45% of obese individuals do not present with poor glycemic and/or lipid profiles, commonly called the metabolically healthy obese (MHO). Concurrently, up to 30% of normal-weight individuals present with cardiometabolic risk factors, the metabolically obese normal-weight (MONW) (Hosseinpanah et al. 2011; Caleyachetty et al. 2017; Arnlöv et al. 2010; Aung et al. 2014; Calori et al. 2011; Wildman et al. 2008; Primeau et al. 2011; Yaghootkar et al. 2014). Accordingly, there is a need to identify risk markers that can identify patient populations at increased risk for, but not presenting with typical characterized associated with T2D. Likewise, there is a need for new therapeutic targets that can help treat T2D in general, and in patient populations possessing MONW/MOH risk loci in particular.

The majority of genetic loci identified through genome-wide association studies (GWAS) map to more than one disease or trait (Watanabe et al. 2019). This highlights extensive pleiotropy between human diseases and traits and suggests that most loci act through multiple cell types and tissues giving rise to complex disease phenotypes. However, the mechanisms that ultimately converge to modulate disease susceptibility of seemingly unrelated traits and complex diseases are unclear. Type 2 Diabetes is a particularly heterogenous disease with hundreds of loci associated (Mahajan et al. 2018) and multiple tissues implicated in mediating genetic susceptibility (Torres et al. 2020). During disease pathogenesis, T2D) manifests as hyperglycemia which results from either a loss of insulin secretion from pancreatic beta-cells and/or a lack of insulin response in peripheral tissues, such as liver, adipose, and skeletal muscle. Disease heterogeneity of T2D gains further complexity by its diverse clinical presentation. Although T2D is more frequent in obese patients, there is growing evidence for a subset of patients presenting with T2D despite otherwise normal weight or even lower weight (Udler et al. 2018).

Citation or identification of any document in this application is not an admission that such a document is available as prior art to the present invention.

### SUMMARY

In one aspect, the present invention provides for a method of treating subjects at risk for, or suffering from a metabolic disease comprising, administering, to a subject in need thereof, a therapeutically effective amount of one or more agents that: increases the expression or activity of COBLL1, BCL2, or KDSR in one or more lipid-accumulating cells; reduces the expression or activity of VPS4B in one or more lipid-accumulating cells; enhances actin remodeling in one or more lipid-accumulating cells; or inhibits apoptosis in one or more lipid-accumulating cells.

In certain embodiments, the one or more lipid-accumulating cells is selected from the group consisting of adipocyte progenitors, adipocytes, and skeletal muscle. In certain embodiments, the metabolic disease is T2D, MONW/MOH, lipodystrophy, insulin resistance with a “lipodystrophy-like” fat distribution, insulin sensitivity, BMI-adjusted T2D, and/or increased BMI-adjusted waist-to-hip ratio (WHIRadjBMI). In certain embodiments, the subject has decreased expression of COBLL1 in adipocytes and/or adipocyte progenitors; decreased expression of BCL2 and/or KDSR in adipose-derived mesenchymal stem cells (AMSCs); decreased expression of BCL2 in skeletal muscle; and/or increased expression of VPS4B in AMSCs.

In certain embodiments, the subject has an impairment of actin cytoskeleton remodeling in adipocytes and/or adipocyte progenitors; and/or comprises one or more MONW/MOH risk loci, preferably, the rs6712203 variant. In certain embodiments, the subject has decreased expression of BCL2 and/or KDSR in AMSCs, decreased expression of BCL2 in skeletal muscle, increased expression of VPS4B in AMSCs, and/or increased apoptosis in adipocytes; and/or comprises one or more lipodystrophy risk loci, preferably, the rs12454712 variant.

In certain embodiments, the one or more agents that enhances actin remodeling is selected from the group consisting of geodiamolides (Geodiamolide H), Jasplakinolide, Chondramide (Chondramide A), ADF/Cofilin, Arp2/3 complex, Profilin, Gelsolin (Flightless-I), Formin, Villin (Advillin), and Adseverin. In certain embodiments, the metabolic disease is Type-2 Diabetes (T2D) and/or MONW/MOH.

In certain embodiments, the one or more agents that inhibits apoptosis is selected from the group consisting of Ginkgo biloba extract (EGb 761), Rhodiola crenulata extract (RCF), salidroside, dehydroepiandrosterone, allopregnanolone, diosmin, glycine, M50054, BI-6C9, TC9-305 (2-sulfonyl-pyrimidinyl derivatives), BI-11A7, 3-o-tolylthiazolidine-2,4-dione, minocycline, methazolamide, melatonin, gamma-tocotrienol (GTT), 3-hydroxypropyl-triphenylphosphonium (TPP)-conjugated imidazole-substituted oleic acid (TPP-IOA), TPP-conjugated stearic acid (TPP-ISA), TPP-6-ISA, CLZ-8, Xanthan gum (XG), PD98059, Vitamin E, and Tanshinone. In certain embodiments, the metabolic disease is lipodystrophy, insulin resistance with a “lipodystrophy-like” fat distribution, insulin sensitivity, BMI-adjusted T2D), increased BMI-adjusted waist-to-hip ratio (WHRadjBMI), and/or Type-2 Diabetes (T2D)).

In certain embodiments, the expression or activity of COBLL1 is increased in adipocyte progenitors or adipocytes. In certain embodiments, the metabolic disease is Type-2 Diabetes (T2D) and/or MONW/MOH.

In certain embodiments, the expression or activity of BCL2 or KDSR is increased in adipocyte progenitors. In certain embodiments, the adipocyte progenitors are subcutaneous adipose-derived mesenchymal stem cells (AMSCs). In certain embodiments, the expression or activity of BCL2 is increased in skeletal muscle. In certain embodiments, the expression or activity of VPS4B is reduced in adipocyte progenitors. In certain embodiments, the adipocyte progenitors are visceral AMSCs. In certain embodiments, the metabolic disease is lipodystrophy, insulin resistance with a “lipodystrophy-like” fat distribution, insulin sensitivity, BMI-adjusted T2D, increased BMI-adjusted waist-to-hip ratio (WHRadjBMI), and/or Type-2 Diabetes (T2D).

In certain embodiments, the one or more agents are one or more small molecules that enhances the activity or expression of COBLL1. In certain embodiments, the one or more agents are one or more small molecules that enhances the activity or expression of BCL2 or KDSR. In certain embodiments, the one or more agents are one or more small molecules that reduces the activity or expression of VPS4B.

In certain embodiments, the one or more agents is a polynucleotide comprising a sequence encoding COBLL1. In certain embodiments, the polynucleotide is part of a vector system comprising adipocyte specific regulatory sequences for tissue- and/or cell type-specific expression of the one or more agents. In certain embodiments, the vector system comprises a viral vector system. In certain embodiments, the viral vector system has tropism for adipose tissue. In certain embodiments, the one or more agents is a recombinant polypeptide derived from the COBLL1 gene or functional variant thereof.

In certain embodiments, the one or more agents is a fusion protein, comprising a DNA binding element of a programmable nuclease configured to specifically bind to a sequence in proximity or distant to the COBLL1 gene and wherein the protein activates expression of COBLL1; or configured to specifically bind to a sequence in proximity or distant to the 18q21.33 locus and wherein the protein activates expression of BCL2 and/or KDSR. In certain embodiments, the DNA-binding portion comprises a zinc finger protein or DNA-binding domain thereof, TALEN protein or DNA-binding domain thereof, or a Cas nuclease protein or DNA-binding domain thereof. In certain embodiments, the DNA-binding portion is linked to an activation domain. In certain embodiments, the activation domain is derived from an alternative splicing variant of POU2F2 that activates expression. In certain embodiments, the fusion protein is encoded in a polynucleotide vector. In certain embodiments, the vector system comprises adipocyte specific regulatory sequences for tissue specific expression of the one or more agents. In certain embodiments, the vector system comprises a viral vector system optionally comprising a tropism for adipose tissue.

In another aspect, the present invention provides for a method of treating subjects suffering from or at risk of developing T2D or lipodystrophy, comprising administering a gene editing system that corrects one or more genomic variants that decrease the expression or activity of COBLL1 in adipocytes and/or adipocyte progenitors; or that decrease the expression or activity of BCL2 and KDSR in adipocyte progenitors, decrease the expression or activity of BCL2 in skeletal muscle, and increase the expression or activity of VPS4B in adipocyte progenitors.

In another aspect, the present invention provides for a method of treating subjects suffering from or at risk of developing a metabolic disease, comprising administering a gene editing system that corrects one or more genomic risk variants selected from the group consisting of rs6712203 (COBLL1 locus), rs9686661, rs4804833, rs2972144, rs13389219, rs11837287, rs7903146 (TCF7L2 locus), rs1534696 (SNX10 locus), rs287621, rs1412956, rs13133548, rs11667352, rs12454712 (BCL2 locus), rs673918, rs646123, rs2963449, rs1572993, rs632057, rs11637681, rs6063048, rs7660000, rs1421085, rs7258937, rs9939609, rs998584, rs4925109, rs12641088, and any variant that is within the haplotype for the above variants.

In certain embodiments, the gene editing system is a zinc finger nuclease, a TALEN, a meganuclease, or a CRISPR-Cas system. In certain embodiments, the gene editing system is a CRISPR-Cas system. In certain embodiments, the method further comprises a donor template, configured to replace a portion of a genomic sequence comprising the one or more genomic risk variants with a wild-type or non-risk variant. In certain embodiments, the one or more variants comprises rs6712203 or rs12454712.

In certain embodiments, the gene editing system is a base editing system that corrects one or more of the genomic variants to a wild type or non-risk variant. In certain embodiments, the base editing system is a CRISPR-Cas base editing system. In certain embodiments, the one or more genomic variants include rs6712203 or rs12454712.

In certain embodiments, a C allele/risk genotype of rs6712203 is edited to the T allele/non-risk genotype; or wherein a T allele/risk genotype of rs12454712 is edited to the C allele/non-risk genotype.

In certain embodiments, the gene editing system is a prime editing system that corrects one or more of the genomic variants to a wild type or non-risk variant. In certain embodiments, the one or more genomic variants include rs6712203 or rs12454712. In certain embodiments, the PEG RNA encodes a donor template to replace the rs6712203 or rs12454712 variant with a wild-type or non-risk variant. In certain embodiments, the gene editing system is a prime editing system and wherein the PEG RNA encodes a donor template to replace the one or more genomic risk variants with a wild type or non-risk variant.

In certain embodiments, the gene editing system is a programmable transposition system that corrects one or more of the genomic variants to a wild type or non-risk variant. In certain embodiments, the one or more genomic variants include rs6712203 or rs12454712. In certain embodiments, the programmable transposition system is a CAST system. In certain embodiments, the guide polynucleotide of the CAST system comprises a donor construct comprising a donor sequence to replace a genomic region comprising the rs6712203 or rs12454712 variant with a wild type sequence.

In another aspect, the present invention provides for a method of treating Type-2 Diabetes in subjects comprising one or more variants that decrease COBLL1 expression or activity by decreasing binding of POU2F2 to a binding site in an enhancer regulating COBLL1 expression comprising, administering to a subject in need thereof 1) allogenic adipocyte progenitors that exhibit wild type COBLL1 expression, or 2) autologous adipocyte progenitors genetically edited to correct the one or more variants to a wild-type sequence.

In another aspect, the present invention provides for a method of treating a metabolic disorder in subjects comprising administering to a subject in need thereof 1) allogenic adipocyte progenitors that do not comprise one or more genomic risk variants selected from the group consisting of rs6712203 (COBLL1 locus), rs9686661, rs4804833, rs2972144, rs13389219, rs11837287, rs7903146 (TCF7L2 locus), rs1534696 (SNX10 locus), rs287621, rs1412956, rs13133548, rs11667352, rs12454712 (BCL2 locus), rs673918, rs646123, rs2963449, rs1572993, rs632057, rs11637681, rs6063048, rs7660000, rs1421085, rs7258937, rs9939609, rs998584, rs4925109, rs12641088, and any variant that is within the haplotype for the above variants; or, 2) autologous adipocyte progenitors genetically edited to correct the one or genomic risk variants to a wild-type or non-risk variant. In certain embodiments, the one or more variants comprise rs6712203 or rs12454712.

In certain embodiments, the adipocyte progenitors are adipose-derived mesenchymal stem cells (AMSCs). In certain embodiments, the autologous adipocyte progenitors are edited to change a C allele/risk genotype of rs6712203 to the T allele/non-risk genotype.

In another aspect, the present invention provides for a method for detecting a variant in subject, comprising, detecting whether a rs6712203 or rs12454712 variant is present in a subject by conducting a genotyping assay on a biological sample from the subject and detecting whether the rs6712203 or rs12454712 variant is present. In certain embodiments, genotyping is conducted by restriction fragment length polymorphism identification, random amplified polymorphic detection, amplified fragment length polymorphism, PCR, DNA sequencing, allele specific oligonucleotide hybridization, or microarray hybridization. In certain embodiments, the method further comprises administering a) a therapeutically effective amount of one or more agents that increase the expression or activity of COBLL1, or enhance actin remodeling in adipocytes or adipocyte progenitors, b) a therapeutically effective amount of one or more agents that increase the expression or activity of BCL2 and/or KDSR, or inhibit apoptosis in adipocytes or adipocyte progenitors, c) a gene editing system that corrects the one or more variants to a wild type sequence, d) adoptive cell transfer comprising allogenic adipocyte or adipocyte progenitor donors exhibiting wild type COBLL1 expression, or autologous adipocyte or adipocyte progenitor donors genetically modified to correct the one or more variants to a wild type sequence, or c) adoptive cell transfer comprising allogenic adipocyte progenitor donors exhibiting wild type BCL2 and/or KDSR expression, or autologous adipocyte progenitor donors genetically modified to correct the one or more variants to a wild type sequence.

In another aspect, the present invention provides for a method of treating T2D comprising: performing a genotyping assay on a biological sample from a subject to determine if the subject has one or more variants that decrease COBLL1 expression or activity by decreasing binding of POU2F2 to a binding site in an enhancer regulating COBLL1 expression; and if the subject has the one or more variants administering a) a therapeutically effective amount of one or more agents that increase the expression or activity of COBLL1, or enhance actin remodeling in adipocytes or adipocyte progenitors, b) a gene editing system that corrects the one or more variants to a wild type sequence, or c) adoptive cell transfer comprising allogenic adipocyte donors exhibiting wild type COBLL1 expression, or autologous adipocyte donors genetically modified to correct the one or more variants to a wild type sequence; or if the subject does not have the one or more variants, administering a standard-of-care T2D therapy.

In another aspect, the present invention provides for a method of treating lipodystrophy comprising: performing a genotyping assay on a biological sample from a subject to determine if the subject has one or more variants that decrease the expression or activity of BCL2 and KDSR in adipocyte progenitors, decrease the expression or activity of BCL2 in skeletal muscle, and increase the expression or activity of VPS4B in adipocyte progenitors; and if the subject has the one or more variants administering a) a therapeutically effective amount of one or more agents that increase the expression or activity of BCL2 and/or KDSR, or inhibit apoptosis in adipocytes or adipocyte progenitors, b) a gene editing system that corrects the one or more variants to a wild type sequence, or c) adoptive cell transfer comprising allogenic adipocyte progenitor donors exhibiting wild type BCL2 and/or KDSR expression, or autologous adipocyte progenitor donors genetically modified to correct the one or more variants to a wild type sequence; or if the subject does not have the one or more variants, administering a standard-of-care lipodystrophy therapy.

In another aspect, the present invention provides for a method for diagnosing metabolically obese normal weight (MONW) subjects at increased risk for developing T2D comprising, detecting one or more variants that decrease the expression or activity of COBLL1 in adipocyte and/or adipocyte progenitors and diagnosing the subject, and diagnosing the subject as increased risk of T2D if the one or more variants are detected. In certain embodiments, the one or more variants decrease binding of POU2F2 to a binding site in an enhancer regulating COBLL1 expression. In certain embodiments, the one or more variants comprises rs6712203.

In another aspect, the present invention provides for a method for diagnosing lipodystrophy subjects at increased risk for developing T2D or heart disease comprising, detecting one or more variants that that decrease the expression or activity of BCL2 and KDSR in adipocyte progenitors, decrease the expression or activity of BCL2 in skeletal muscle, and increase the expression or activity of VPS4B in adipocyte progenitors and diagnosing the subject as increased risk of T2D or heart disease if the one or more variants are detected. In certain embodiments, the one or more variants comprises rs12454712.

In another aspect, the present invention provides for a method of screening for agents capable of treating T2D in subjects with a MONW/MOH risk phenotype comprising: treating a population of cells comprising adipocytes having the rs6712203 variant with an agent; and detecting actin remodeling and/or one or more COBLL1 co-regulated genes, wherein detecting an increase in actin remodeling and/or the one or more genes identifies agent as capable of treating T2D) in subjects having a MONW/MOH risk phenotype. In certain embodiments, the one or more COBLL1 co-regulated genes are selected from the group consisting of ITGAM, PIK3CA, ROCK2, ITGA1, ARHGEF7, CRK, FGFR2, and ARHGEF6.

In another aspect, the present invention provides for a method of screening for agents capable of treating lipodystrophy in subjects with a lipodystrophy risk phenotype comprising: treating a population of cells comprising adipocytes having the rs12454712 variant with an agent; and detecting apoptosis and/or one or more apoptosis genes, wherein detecting a decrease in apoptosis and/or one or more apoptosis genes identifies agent as capable of treating lipodystrophy in subjects having a lipodystrophy risk phenotype.

In another aspect, the present invention provides for an unbiased high-throughput multiplex profiling method for simultaneously identifying morphological and cellular phenotypes for lipid-accumulating cells comprising: staining a cellular system comprising one or more lipid-accumulating cells with one or more stains that differentiate cellular compartments selected from the group consisting of nuclei, cytoplasm and total cell and differentiate organelles selected from the group consisting of DNA, mitochondria, actin, Golgi, plasma membrane, lipids, nucleoli and cytoplasmic RNA; imaging the stained cells using an automated image analysis pipeline; and identifying one or more morphological features for each of the organelles from the resulting images, wherein the features comprise one or more features selected from the group consisting of object size, object shape, intensity, granularity, texture, colocalization, number of objects, distance to neighboring objects, cellular compartment, and combinations thereof. In certain embodiments, about 100 or more cells are imaged for the cellular system. In certain embodiments, about 500 or more cells are imaged for the cellular system. In certain embodiments, each feature for each organelle includes a quantitative range comprising at least two values for the feature. In certain embodiments, a pattern of morphological features is linked to a cellular phenotype. In certain embodiments, the morphological features are linked to one or more gene expression programs.

In certain embodiments, the cellular system is obtained from a subject. In certain embodiments, the cellular system comprises lipocytes. In certain embodiments, the lipocytes are selected from the group consisting of adipocytes, hepatocytes, macrophages/foam cells and glial cells. In certain embodiments, the lipocytes are part of a pathophysiological process in cells selected from the group consisting of vascular smooth muscle cells, skeletal muscle cells, renal podocytes, and cancer cells. In certain embodiments, the cellular system comprises stem cells differentiated over a time course, wherein the cells from the cellular system are stained and imaged at different time points. In certain embodiments, the time points comprise one or more time points selected from the group consisting of 0 days, 3 days, 8 days and 14 days. In certain embodiments, the cellular system comprises adipose-derived mesenchymal stem cells (AMSCs) differentiated to adipocytes, wherein the cellular system is stained over a time course. In certain embodiments, the AMSCs are obtained from a subject. In certain embodiments, the AMSCs are subcutaneous AMSCs. In certain embodiments, the AMSCs are visceral AMSCs.

In certain embodiments, the method further comprises performing RNA-seq on the lipid-accumulating cells.

In certain embodiments, the cellular system is stained with one or more fluorescent dyes selected from the group consisting of Hoechst, MitoTracker Red, Phalloidin, wheat germ agglutinin (WGA), BODIPY, and SYTO14. In certain embodiments, the imaging is taken across four channels. In certain embodiments, the image analysis pipeline comprises image analysis software and a novel algorithm.

In certain embodiments, cells are clustered based on patterns of features identified.

In certain embodiments, the imaging pipeline comprises artificial intelligence, machine learning, deep learning, neural networks, and/or linear regression modeling.

In certain embodiments, the cellular system comprises cells comprising a SNP of interest, whereby morphological and cellular phenotypes can be determined for the SNP. In certain embodiments, the cellular system comprises cells perturbed with one or more drugs, whereby morphological and cellular phenotypes can be determined for the one or more drugs. In certain embodiments, the cellular system comprises cells perturbed at one or more genomic loci, whereby morphological and cellular phenotypes can be determined for the one or more genomic loci. In certain embodiments, the cells are perturbed with a programmable nuclease or RNAi.

In another aspect, the present invention provides for a method of identifying morphological features for predicting metabolic clinical characteristics in a subject in need thereof comprising: identifying morphological features according to the method of any embodiment herein for one or more cellular systems derived from one or more subjects having a metabolic clinical characteristic; and fitting a logistic regression model for the clinical characteristic on the entire set of features from and selecting features that best fit the model. In certain embodiments, the method further comprises: identifying a subset of features comprising: constructing an interaction network between the features, wherein nodes represent features, edges indicate interactions between two nodes, and edge weight indicates the strength of the interaction, and selecting a subset of nodes with at least one edge above a cutoff weight, whereby features with high-weight interactions are selected; and fitting a logistic regression model for the clinical characteristic on the entire set of features and selecting features that best fit the model. In certain embodiments, the method further comprises grouping the features into a compartment category selected from the group consisting of lipid, actin/Golgi/plasma membrane (AGP), Mito, DNA, and other, and stratifying by differentiation day, wherein the number of features that can be modeled in every grouped and stratified category are the features.

In another aspect, the present invention provides for a method of predicting metabolic clinical characteristics in a subject in need thereof comprising: identifying morphological or cellular features according to the method of any embodiment herein for one or more cellular systems derived from the subject; and estimating a metabolic clinical characteristic from one or more of the features. In certain embodiments, the one or more features used for estimating the clinical characteristic are selected according to any embodiment herein.

In another aspect, the present invention provides for a method of identifying histological features for predicting metabolic clinical characteristics in a subject in need thereof comprising: identifying features for one or more histological images of adipose tissue samples obtained from one or more subjects having a metabolic clinical characteristic, wherein the features are identified by a method comprising: grouping at least 100-500 cells from an image into cell area (μm²) categories, wherein the categories are defined by cell area ranges for a plurality of control subjects of the same sample tissue type; determining for each cell area category one or more features selected from: the fraction of cells in the cell area category, median area of cells in the category, 25% interquartile point in the category, and 75% interquartile point in the category; and fitting a logistic regression model for the clinical characteristic on the entire set of features and selecting features that best fit the model. In certain embodiments, the cells are grouped into 5 area categories consisting of: a cell area <25% quartile point for the control group (very small), a cell area ≥25% quartile point for the control group and <the median cell area for the control group (small), a cell area ≥median cell area for the control group and <mean cell area for the control group (medium), a cell area ≥mean area for the control group and <75% quartile point for the control group (large), and a cell area ≥75% quartile point for the control group (very large).

In another aspect, the present invention provides for a method of predicting metabolic clinical characteristics in a subject in need thereof comprising: identifying features from a histological image of an adipose tissue sample obtained from the subject comprising: grouping at least 100-500 cells from the image into cell area (μm²) categories, wherein the categories are defined by cell area ranges for a plurality of control subjects of the same cell tissue type; determining for each cell area category one or more features selected from the fraction of cells in the cell area category, median area of cells in the category, 25% interquartile point in the category, and 75% interquartile point in the category; and estimating a metabolic clinical characteristic from one or more of the features. In certain embodiments, the cells are grouped into 5 area categories consisting of: a cell area <25% quartile point for the control group (very small), a cell area ≥25% quartile point for the control group and <the median cell area for the control group (small), a cell area ≥median cell area for the control group and <mean cell area for the control group (medium), a cell area ≥mean area for the control group and <75% quartile point for the control group (large), and a cell area ≥75% quartile point for the control group (very large). In certain embodiments, the one or more features used for estimating the clinical characteristic are selected according to any embodiment herein. In certain embodiments, the tissue is subcutaneous adipose tissue. In certain embodiments, the tissue is visceral adipose tissue.

In another aspect, the present invention provides for a method of predicting metabolic clinical characteristics in a subject in need thereof comprising determining clinical characteristics using morphological features and using histological features; and comparing the clinical characteristics to predict clinical characteristics for the subject.

In certain embodiments, the logistic regression model is a linear model with logit link (GLM). In certain embodiments, the linear association with binomial distribution is implemented using the R glm function, wherein the default glm convergence criteria on deviances is used to stop the iterations, wherein the DeLong method is used to calculate confidence intervals for the e-statistics, wherein forward feature selection (R step function) is used to select the features, and/or wherein the Akaike information criterion (AIC) is used as the stop condition for the feature selection procedure.

In another aspect, the present invention provides for a method of detecting HOMA-IR or WHIRadjBMI risk in a subject comprising, detecting one or more features according to the method of any embodiment herein, wherein the one or more features are selected from the group consisting of: increased lipid granularity in visceral adipocytes; increased lipid texture_SumEntropy in visceral adipocytes; increased cell area/shape in visceral adipocytes; decreased lipid texture_InverseDifferenceMoment in visceral adipocytes; decreased BODIPY Texture_AngularSecondMoment; upregulation of one or more genes selected from the group consisting of GYS-1, TPI1, PFKP and PGK; and downregulation of one or more genes selected from the group consisting of ACAA1 and SCP2.

In another aspect, the present invention provides for a method of detecting lipodystrophy risk in a subject comprising, detecting one or more features according to the method of any embodiment herein, wherein the one or more features are selected from the group consisting of: increased mitochondrial stain intensity; smaller lipid droplets on average compared to adipocytes from individuals with low polygenic risk; upregulation of one or more genes selected from the group consisting of EHHADH and NFATC3.

In certain embodiments, the method further comprises a treatment step comprising administering one or more of insulin, thiazolidinedione, biguanide, meglitinide, DPP-4 inhibitors, Sodium-glucose transporter 2 (SGLT2) inhibitor, alpha-glucosidase inhibitor, bile acid sequestrant, sulfonylureas and/or amylin analogs.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1J—LipocyteProfiler creates rich morphological and cellular profiles in adipocytes that are informative for known function a) Schematic of LipocyteProfiler which is a high-content imaging assay that multiplexes six fluorescent dyes imaged in four channels in conjunction with an automated image analysis pipeline to generate rich cellular profiles in lipid-storing cell types, such as adipocytes during differentiation b) Representative microscope image of fully differentiated adipocytes across four channels plus a merged representation across channels c) LipocyteProfiler extracts 3005 morphological and cellular features that map equally to three cellular compartments and across four channels d) BODIPY Median Intensity, a measurement of lipid content within a cell, significantly increases with adipogenic differentiation and decreases following CRISPR/Cas9-mediated knockdown of PPARG in differentiated white adipocytes, see also FIG. 8d knock-out efficiency c) Median mitochondrial intensity is higher in brown (hBAT) compared to white (hWAT) adipocytes throughout differentiation and decreased after CRISPR/Cas9-mediated knockout of PGC1A in hWAT, see also FIG. 8d knock-out efficiency f) BODIPY Granularity measures, as a spectra of 16 lipid droplet size measures, show size-specific changes in hWAT and hBAT during differentiation, see also FIG. 8a Granularity features informative for larger lipid droplets (BODIPY_Granularity 10-16) correlate positively with PLIN1 gene expression and are reduced in PLIN1-KO adipocytes, see also FIGS. 8b-cx-8x PLIN2, FASN-KO, DGAT2-KO g) During adipogenesis of hWAT, cytoskeletal remodeling and resulting homogeneity decreases as shown by reduced Texture_AngularSecondMoment and increased Texture_Entropy of AGP, whereas the inverse is observed for BODIPY where the increase in lipid droplets during differentiation is associated with a more homogenous appearance. hWAT cells show a more homogenous lipid droplet-related appearance than hBAT as seen by higher Texture_AngularSecondMoment and lower Texture_Entropy in hWAT compared to hBAT h) CRISPR/Cas9-mediated knockout of MFN1, a mitochondrial fusion gene, changes Mitochondria_Texture_InfoMeas1, a measure of spatial relationship between specific intensity values, see also FIG. 8c knock-out efficiency i) Large BODIPY objects informative for large lipid droplets are absent in the progenitor state and in early differentiation and increase in later stages of differentiation and are reduced CRISPR/Cas9-mediated KO of PPARG, at day 14 of differentiation, see also FIG. 8c knock-out efficiency j) Area, Shape and Size intuitively change over the course of hWAT differentiation as cells become lipid-laden, grow in size and nuclei become less compact.

FIG. 2A-2C—Correlations between morphological and transcriptional profiles a) Linear mixed model (LMM) was applied to correlate 2760 morphological features derived from LipocyteProfiler with 60,000 transcripts derived from RNAseq in matched samples of subcutaneous AMSCs at terminal differentiation. With FDR <0.1%, Applicants discover 44,736 non-redundant connections that map to 869 morphological features and 10931 genes b) Network of transcript-feature correlations. Genes correlated to specific features are enriched for pathways plausible for their meaning using Pathway enrichment analyses c) morphological signatures of adipocyte marker genes SCD, PLIN2, LIPE, TIMM22, INSR and GLUT4 recapitulate their cellular function.

FIG. 3A-3G—LipocyteProfiler identifies distinct depot-specific morphological signatures associated with differentiation trajectories in both visceral and subcutaneous AMSCs a) Human adipose-derived mesenchymal stem cells (AMSCs) isolated from subcutaneous and visceral adipose depots were differentiated for 14 days, LipocyteProfiler and RNAseq was performed throughout differentiation b) Morphological and transcriptomic profiles show time course specific signatures revealing a differentiation trajectory, but only morphological profiles generated by LipocyteProfiler additionally resolve adipose depot-specific signatures c) Subcutaneous and visceral AMSCs at terminal differentiation have distinct morphological and cellular profiles with differences that are spread across all channels, see also FIG. 9b time-points 1-3 d) SDP analysis. Proportion of subgroups of features driving differentiation differ between subcutaneous and visceral adipocytes and dynamically change over the course of differentiation. In both depots, mitochondrial features drive differentiation predominantly in the early phase of differentiation whereas BODIPY-related features predominate in the terminal phases e) The number of lipid droplets is higher in subcutaneous AMSCs compared to visceral AMSCs at terminal differentiation f) The amount of lipid droplets is higher in subcutaneous and visceral AMSCs at terminal differentiation g) BODIPY granularity from subcutaneous AMSCs at day 14 of differentiation correlates positively with adipose tissue-derived adipocyte size, but shows the inverse relationship for visceral adipose tissue, suggesting distinct cellular mechanisms that lead to adipose tissue hypertrophy in these two depots.

FIG. 4A-4I—LipocyteProfiler identifies molecular mechanisms of drug stimulation in adipocytes and hepatocytes a) LipocyteProfiler was performed in visceral AMSCs that were treated with isoproterenol, which acts on ADRB to induce lipolysis in adipocytes, for 24 hours b) Isoproterenol treatment results in lipid-related and mitochondrial morphological changes in visceral AMSCs at day 14 of differentiation, see also FIG. 9c subcutaneous c) isoproterenol treatment of visceral AMSCs increases mitochondrial intensity and texture entropy, while both measures decrease for BODIPY, mimicking a morphological profile seen during activation of an adipocyte browning program d) The expression of the lipolytic marker gene HSL correlates negatively with Texture SumEntropy e) Isoproterenol treatment reduces lipid droplet sizes measured via BODIPY Granularity f) Oleic acid treatment in hepatocytes shows BODIPY enriched morphological and cellular profile g) Oleic acid treatment alters lipid-related morphological features suggestive of increased lipid droplet size and number h) The effect of metformin treatment in hepatocytes on LipocyteProfile is spread across all channels i) Metformin effect in hepatocytes is suggestive of increased mitochondrial activity, while lipid droplet size and number are reduced. Metformin-treated hepatocytes are also smaller and show reduced cytoskeletal randomness.

FIG. 5A-5F—Polygenic risk effects for insulin resistance converges on a lipid rich morphological profile in differentiated visceral adipocytes a) Donors from the bottom and top 25 percentiles of genome-wide polygenic risk scores for three T2D-related traits (HOMA-IR, T2D, WHIRadjBMI) were selected to compare LipocyteProfiles across the time course of visceral and subcutaneous adipocyte differentiation b) There are significant polygenic effects on image-based cellular signatures for HOMA-IR in terminally differentiated visceral AMSCs (largely BODIPY features) and WHIRadjBMI in subcutaneous AMSCs at day 14 of differentiation (largely mitochondrial and BODIPY features), but no effect for T2D, see also FIG. 11a+c time-points 1-3 c) HOMA-IR polygenic risk in visceral AMSCs manifested in altered BODIPY texture features and a LipocyteProfile resembling an inhibition of lipolysis and lipid degradation, see also FIG. 4c d-f) Correlation of gene expression of 512 genes known to be involved in adipocyte function with HOMA-IR PRS showed that genes that correlated with HOMA-IR were enriched for biological processes related to glucose metabolism, fatty acid transport, degradation and lipolysis (KEGG Pathways 2019).

FIG. 6A-6D—Polygenic risk for lipodystrophy-like phenotype manifests in cellular programs that indicate reduced lipid accumulation capacity in subcutaneous adipocytes a) Schematic of T2D process specific PRS. lipodystrophy-specific PRS consists of 20 T2D loci contributing to polygenic risk for a lipodystrophy-like phenotype, refer Udler et al. b) Depot-specific effects on LipocyteProfiles in AMSCs at day 14 are under the polygenetic control of lipodystrophy cluster with a mitochondrial and AGP driven profile in subcutaneous AMSCs, whereas in visceral AMSCs mostly BODIPY features were associated with increased polygenic risk c) Representative images of computed averaged subcutaneous AMSCs from low and high risk allele carriers for lipodystrophy PRS show higher mitochondrial intensity and reduced lipid droplet size in high risk carriers, see also FIG. 12a time-points 1-3 d) Genes that are connected to lipodystrophy PRS-mediated differential features in subcutaneous AMSCs at day 14 are enriched for mitochondrial LipocyteProfiler features, see also FIG. 12c.

FIG. 7A-7G—Allele-specific effect of the 2p23.3 lipodystrophy locus on mitochondrial fragmentation and lipid accumulation in visceral adipocytes a) PheWAS at the DNMT34 risk locus shows associations with height, WHRadjBMI, T2D and Calcium b) LipocyteProfiler was performed in subcutaneous and visceral AMSCs of 8 risk and 6 non-risk haplotype carriers at 3 time points during adipocyte differentiation (day 0, 3 and 14) c) In visceral AMSCs, 74 and 76 features were different between haplotypes at day 3 and day 14 of differentiation, respectively, in visceral AMSCs, with 70% of differential features at day 3 being mitochondrial, whereas 80% of those features different at day 14 were BODIPY-related d) Mitochondrial Max Intensity and Texture Entropy were higher at day 3 of differentiation in visceral AMSCs from 6 risk haplotype carriers, suggesting more fragmented and more active mitochondria e) LargeBODIPYObject Median Intensity was lower and Texture AngularSecondMoment was higher at day 14 of differentiation in visceral AMSCs from 6 risk haplotype carriers, suggesting a perturbed lipid phenotype of reduced lipid droplet stabilization and/or formation f) Mitochondrial Granularity (7-8 size measures) at day 3 of differentiation in visceral AMSCs was increased in risk allele carriers, suggestive of less tubular and more active mitochondria g) BODIPY Granularity was reduced in visceral AMSCs of risk allele carriers at day 14 of differentiation, suggesting smaller sized lipid droplets.

FIG. 8A-8C—Benchmarking of Lipocyte Profiler features a) BODIPY Granularity measurements, captured by spectra of 16 lipid droplet size measures, show size-specific changes in hWAT and hBAT during differentiation, suggesting hBAT generally accumulate less medium-size and large lipid droplets as seen by lower values across the spectra of granularity b) Granularity features informative for larger lipid droplets (BODIPY_Granularity 10-16) correlate positively with PLIN2 gene expression c) BODIPY_Granularity measures are reduced in CRISPR/Cas9-mediated KO of FASN and DGAT in hWAT at day 14 of differentiation.

FIG. 9A-9D—Depot and drug induced differences in adipocytes and hepatocytes a) Gene expression from RNAseq of adipogenesis marker genes LIPE, PPARG, PLIN1 and GLUT4 in visceral (top) and subcutaneous (bottom) AMSCs throughout differentiation b) Subcutaneous and visceral AMSCs have distinct morphological and cellular profiles with differences that are spread across all channels that become apparent at day 3 of differentiation and are maintained at day 8, see also FIG. 3c time-point day 14 c) Isoproterenol treatment results in no effect on morphological profile in subcutaneous AMSCs at day 14 of differentiation, see also FIG. 4b visceral d) ADRB3 is higher expressed in visceral compared to subcutaneous adipose tissue from GTEX.

FIG. 10A-10B—Batch effect and variance explained analysis a) Morphological profiles of hBAT, hWAT and SGBS across differentiation cluster according to cell type and show maturation trajectory in PC1 and PC2, but don't cluster in batch distinct groups (two plots on left). BEclear analysis shows no significant batch effect and accuracy of predicting cell type is higher than predicting batch using a k-nearest neighbor supervised machine learning algorithm, two plots on right b) Variance component analysis across all data to assess contribution of intrinsic genetic variation on adipocyte morphology and cellular traits across 65 donor-derived differentiating AMSCs. This analysis showed that patientID explains more of overall variance compared to contribution of other possible confounding factors such as batch, adipose depot, T2D status, age, sex, BMI, cell density, month/year of sampling and passage numbers.

FIG. 11A-11C—PRS a) LipocyteProfiler differences between top and bottom 25% of HOMA-IR risk in subcutaneous and visceral AMSCs at day 0, day 3 and day 8 of adipogenesis, see also FIG. 5b, day 14 visceral b) Representative significant features of HOMA-IR morphological profile correlate with PRS percentile, see also FIG. 5c c) LipocyteProfiler differences between top and bottom 25% of WHIRadjBMI risk in subcutaneous and visceral AMSCs at day 0, day 3 and day 8 of adipogenesis, see also FIG. 5b, day 14 subcutaneous.

FIG. 12A-12C—Lipodystrophy a) LipocyteProfiler differences driven by polygenic risk in subcutaneous and visceral AMSCs at day 0, day 3 and day 8 of adipogenesis, see also FIG. 6b, day 14 b) Morphological and cellular profiles of marker genes of monogenic familial partial lipodystrophy syndromes like PPARG, LIPE, PLIN1, AKT2, CIDEC, LMNA and ZMPSTE24 show similar morphological signatures to the polygenic lipodystrophy profile with high effect sizes of mitochondrial and AGP features, see also FIG. 6b, day 14 c) Genes that were identified to be correlated to lipodystrophy morphological profile are under polygenic control for lipodystrophy risk.

FIG. 13—DNMT3A-KO mice. Heterozygous knockout mice for DNMT3A show increased body weight due to increased overall fat mass and have reduced bone mineral density.

FIG. 14A-14C—The pleiotropic 2q24.3 MONW locus is associated with increased risk for type 2 diabetes and decreased adiposity related traits and maps to sparse enhancer signatures in adipocytes. a) PheWAS of trait associations at the haplotype in UK Biobank (Elliott et al. 2017). Colors represent trait classes while individual variant association p-values are shown on the Y axis. b) The 2q24.3 MONW locus spans 23 non-coding genetic variants in high linkage disequilibrium. The region of association localizes to a >55 kb interval in an intergenic region between the COBLL1 and the GRB14 genes. c) Chromatin state annotations for the 55 kb-long MNOW risk locus. Genomic intervals are shown across 127 human cell types and tissues reference epigenomes profiled by the Roadmap Epigenomics projects, based on a 25-state chromatin state model (colors, see FIG. 19) learned from 12 epigenomic marks using imputed signal tracks at 25-nucleotide resolution (Roadmap Epigenomics Consortium et al., 2015). Chromatin states considered here include Polycomb repressed states (grey, H3K27me3), weak enhancers (yellow, H3K4me1 only), strong enhancers (orange, also H3K27ac), and transcribed enhancers (lime, also H3K36me3). Polycomb-repressed segments in mesenchymal cells are denoted with a dotted red box.

FIG. 15A-15F—Sequence-based computational methods predict rs6712203 as a likely causal variant at the 2q24.3 MONW locus. a) Phylogenetic conservation analysis and deep convolutional neural network (CNN)-based prediction of chromatin accessibility for 19 highly linked (LD=r2>0.6) variants at the 2q24.3 locus. X axis: Phylogenetic conservation scores of jointly conserved motifs using PMCA (Claussnitzer et al., 2014). PMCA was used to identify orthologous regions in 20 vertebrate species and to scan the 120 bp sequence context around each variant in the haplotype for groups of transcription factor binding site motifs whose sequence, order and distance range is cross-species conserved. The scores have a minimum of 0 (no conserved motif modules), with scores indicating the count of non-overlapping jointly conserved transcription factor binding site motifs whose relative positions within the window are conserved. Y axis: Predicted relative change in chromatin accessibility (SNP accessibility difference SAD scores) in adipocytes for each SNP comparing alleles on each SNP comparing alleles on haplotype 1 and haplotype 2. A deep CNN Basset (Kelley et al., 2016) was trained on genome-wide ATAC-seq data assayed in terminally differentiated AMSCs (day 24 of adipogenic differentiation, see methods). Alleles were assigned to each SNP in the haplotype and evaluated for predicted accessibility using Basset, in which more positive numbers indicate more predicted accessibility on the alternative allele compared to the reference allele. Both PMCA and Basset highlight rs6712203 as a likely causal variant at the 2q24.3 locus and predict that rs6712203 T allele increases chromatin accessibility. b) The haplotype spans a region downstream and intronic to COBLL1. The prioritized variant, rs6712203, lies in the intronic region of the haplotype. b) For the C allele at the locus, there are no substantial nucleotide variants which reduce binding in the region of the SNP rs6712203. Each position on the X axis represents a single nucleotide in the vicinity of COBLL1 and the four values in the heatmap correspond to substitutions to each of the four possible bases. c) For the T allele at the locus, in silico saturation mutagenesis suggests that loss of binding in the region of the POU2F motif that overlaps rs6712203, including the C allele itself, result in significantly reduced predicted chromatin accessibility at the locus. d) Intragenomic replicates (Cowper-Sal lari et al. 2012) predicts a substantially higher binding affinity of POU2 family transcription factors for the T allele than C allele to both strands. X axis, offset from instances of the given kmer sequence (as shown by color); Y axis, estimated affinity of binding in the region. Model with 8mers shown; alternatives with 6mers through 9mers are in FIG. 21b. c) EMSA of nuclear extract of differentiated adipocytes indicates substantially higher binding affinity to the T allele of rs6712203 than the C allele. Competition experiment shown in FIG. 21c. f) Generation of isogenic AMSCs with genotype CC at rs6712203 starting from a TT homozygote. Isogenic lines were differentiated to adipocytes after undergoing clonal expansion, and POU2F2 was silenced (siPOU2F2) or not (siNT).

FIG. 16A-16N—The 2q24.3 effector gene COBLL1 affects actin remodeling processes in differentiating adipocytes and subsequently adipocyte differentiation, insulin sensitivity and lipolysis rate. a) KEGG pathway enrichment of genes correlated with COBLL1 in differentiating adipocytes. Genes with significant co-expression with COBLL1 across four differentiation timepoints in 30 donors were tested for enrichment in KEGG pathways using Enrichr (Chen et al. 2013; Kuleshov et al. 2016). Those pathways which were FDR-adjusted significantly enriched are shown in red. Wikipathways and HCI pathways for differentiating adipocytes, as well as co-expression analysis in an independent set of tissue samples from 12 lean and obese individuals, are shown in FIG. 22c-e. b) Schematic of siCOBLL1 experiments in primary human AMSCs across differentiation. Human AMSCs from a normal-weight female donor were silenced 3 days prior to induction of adipogenesis and Adipocyte Painting performed at 4 time points of differentiation (day 0, day 3, day 8 and day 14) c) Morphological profiles of siCOBLL1-compared to siNT-treated AMSCs at day 14 of differentiation (t-test, 5% FDR). d) Pie chart illustrating non-redundant differential features per channel and class of measurement comparing siCOBLL1 and siNT control at day 14 of differentiation. c-f) Spatial Intensity Distribution of AGP in the center of the cytoplasm (e; Cytoplasm_RadialDistribution_FracAtD_AGP_1of4) and juxtaposed to the plasma membrane (f; Cytoplasm_RadialDistribution_RadialCV_AGP_4of4), t-test g) Representative microscopic images of COBLL1 KD and control at day 0 and 14 of differentiation. h-i) Texture of BODIPY stain (h; Cells Texture Correlation_BODIPY_10_01) and granularity of BODIPY stain (i; Cells_Granularity_3_BODIPY) of siCOBLL1 KD and siNT AMSCs throughout differentiation; t-test. j) Oil-Red-O lipid staining in differentiated SGBS adipocytes following stable lentiviral knock-down of COBLL1 (shCOBLL1) versus the empty vector control (shEV). k) GPDH metabolic activity test in differentiated shCOBLL1 adipocytes compared to shNT adipocytes, t-test. I) Basal and insulin-stimulated ³H-2-deoxyglucose uptake in differentiated shCOBLL1 adipocytes compared to shEV adipocytes, one-way ANOVA with Tukey's HISD test. m) Basal and isoproterenol-stimulated lipolysis rate as measured by glycerol release in differentiated shCOBLL1 adipocytes compared to shEV adipocytes, one-way ANOVA with Tukey's HISD test. n) Western blots for lipolysis-relevant proteins assayed in basal or isoproterenol/IBMX stimulated differentiated shCOBLL1 compared to shEV SGBS adipocytes.

FIG. 17A-17H—The rs6712203 MONW risk haplotype affects the actin remodeling process in adipocytes and adipocyte lipid storage capacity. a) Schematic of adipocyte differentiation and adipocyte profiling of AMSCs derived from TT (n=7) and CC (n=6) allele carriers of rs6712203 using Adipocyte Profiler. b-c) Differences in morphological profiles between TT (n=7) and CC (n=6) allele carriers at day 14 in visceral (b) and subcutaneous (c) AMSCs (multi-way ANOVA, significance level 5% FDR). d) Pie chart illustrating non-redundant differential features per channel and class of measurement at day 14 of subcutaneous adipocyte differentiation in rs6712003 homozygous risk compared to non-risk carriers. e-f) Spatial Intensity Distribution of AGP in the center of the cytoplasm (e; Cytoplasm_RadialDistribution_FracAtD_AGP_1of4) and juxtaposed to the plasma membrane (f; Cytoplasm_RadialDistribution_RadialCV_AGP_4of4) throughout differentiation, multi-way ANOVA. g-h) Lipid droplet count (g; Cells_Children_LargeBODIPYObjects_Count) and intensity of BODIPY stain (h; Cells_Intensity_IntegratedIntensity_AGP) throughout differentiation, multi-way ANOVA.

FIG. 18A-18O—Cobll1 deficient mice are leaner and display metabolically dysfunctional phenotypes. a) Schematic of differentiation and Adipocyte Profiling at 3 time points (day 0, day 2, day 10) of AMSCs derived from Cobll1−/− mice (n=3) and WT (n=4). b) Morphological profiles of AMSCs of Cobll1−/− mice compared to AMSCs of WT mice at day 10; t-test, 5% FDR. c) Pie chart illustrating non-redundant differential features per channel and class of measurement comparing AMSCs of Cobll1−/− and WT mice at day 10 of differentiation. d-g) Lipid droplet count (d; Cells_Children_LargeBODIPYObjects_Count), intensity of BODIPY stain (e; Cells_Intensity_IntegratedIntensity_AGP), granularity of BODIPY stain (f; Cells_Granularity_3_BODIPY) and Texture of actin cytoskeleton (g; Cytoplasm_Texture_Entropy_AGP) at day 10 of differentiation. h) Oil red staining of differentiated murine AMSCs. i) GPDH activity of differentiated murine AMSCs was assessed by measuring the decrease in NADH at 340 nm. Data represent mean±SEM. *, P<0.05 compared to WT group. n.s. not significant. j) Representative photograph of 14 week-old WT and Cobll1−/− mice fed a normal chow. Yellow dotted lines delineate perigonadal white adipose tissue pWAT. k) Mouse body weight across time. I) Body composition (Fat mass/Body weight) m) Body length measurements of WT and Cobll1−/− mice (n=6). n) bone mineral density (BMD) analyses by DEXA. o) Intraperitoneal glucose tolerance test (IPGTT) in WT and Cobll1−/− and Cobll1+/− mice. Graph shows the area under the curve (AUC) of the blood glucose concentration levels measured during IPGTT.

FIG. 19A-19D—a) The annotation panel and color key for the twenty-five-state chromatin model (Roadmap Epigenomics Consortium et al., 2015). Rows represent states and columns are emission parameters (left table) and enrichments of relevant genomic annotations (right panel). b) Stranded allele-specific chromatin measures at the haplotype. For each day of differentiation of an individual heterozygous for the haplotype, the number of reads overlapping with 23 non-coding SNPs in the haplotype, ordered by their start position and strand relative to the position of the variant, are shown. More reads indicate more extensive activity at the variants in the haplotype. c) Replication of the effect at time 0) (mesenchymal stem cells) with ATAC-seq. d) BMI dependence on T2D) association with rs6712203.

FIG. 20A-20C—a) Predicted binding of POU2F2 between the two alleles using the Intragenomic Replicate Method (Cowper-Sal⋅lari et al., 2012). As in FIG. 15d with different kmer counts (6-9) show a consistent change in affinity to the POU2 motif canonical kmer in the region. b) Cross-cell type conserved genome-wide higher order chromatin interactions for the 2q24.3 locus analyzed by Hi-C assays in human fibroblasts (left) and NHEK primary normal human epidermal keratinocytes (right), chr2: 163,556,000-167,558,000 (hg19), binned at 2 kb resolution. c) Schematic of the regulatory circuitry under the genetic control of rs6712203.

FIG. 21—Conditional analyses implicating rs6712203 in the genetic control of anthropometric traits and type 2 diabetes. Each panel represents a different trait/sex/conditional analysis window, and all panels have an X axis corresponding to 100 kb on either side of the rs6712203 variant. Y axis shows, for each variant in the window, the association strength for the given trait conditioned on the variants noted in White British participants in UK Biobank with the sex shown, and red lines indicate the significance threshold 5×10⁻⁸).

FIG. 22A-22E—a) COBLL1 expression in subcutaneous and visceral AMSCs throughout differentiation b) COBLL1 gene expression enrichment across 142 tissues from enrichment profiler (Benita et al. 2010). COBLL1 probes 203641 s at and 203642 s at were used for analysis. c) Correlation with COBLL1 probe ILMN_1761260 using microarray data from lean and obese individuals. d-e) Enrichment of pathways in the HCI (d) and WikiPathways (e) gene set lists from Enrichr, plotted as in FIG. 16a, with p-value thresholds corresponding to the FDR cutoffs in those data.

FIG. 23A-23O—a) COBLL1 expression in siCOBLL1 compared to siNT at day 0, day 3 and day 14 of differentiation, t-test. b-d) Morphological profiles of siCOBLL1-compared to siNT-treated AMSCs at day 0 (b) day 3 (c) and day 9 (d) of differentiation (t-test, 5% FDR). e) Schematic of experimental set-up siCOBLL1 KD and AMSCs differentiation. f) qPCR-based gene expression of COBLL1 and adipocyte marker genes GLUT4, FASN, LIPE, PPARG, PLIN1, FABP4, CEBPA, ADIPOQ in siCOBLL1- and siNT-treated AMSCs at day 14 of differentiation, t-test g) UMAP-based dimensionality reduction of Adipocyte Profiler features in siCOBLL1- and siNT-treated AMSCs throughout differentiation. siCOBLL1 KD in preadipocytes (day-3 of differentiation, red siCOBLL-treated, blue siNT-treated), differentiated AMSCs cluster separately in siCOBLL1 and siNT groups at day 9 and day 14 of differentiation. siCOBLL1- and siNT-treated AMSCs at day 9 (yellow=siCOBLL1-treated, green=siNT-treated) of differentiation cluster separately in patient specific clusters at day 9 and day 14. h) Actin and COBLL1 staining in siCOBLL1 compared to siNT differentiated human subcutaneous adipocytes at day 9 using phalloidin and COBLL1 antibody (specification: HPA053344) as first and Alexa-Fluor 488 as second antibody. Hoechst staining was used to identify single cell nuclei, magnification x63/oil. i) qPCR-based leptin gene expression measurement in shCOBLL1 compared to shEV differentiated SGBS adipocytes. Data are represented as median with 95% confidence interval (one-way ANOVA with Tukey's HSD test). j) Correlation (Pearson's r) of COBLL1 mRNA (COBLL1 ILMN_1761260) with LEP mRNA (ILMN_2207504) in human whole subcutaneous adipose tissue from 24 lean individuals measured by Illumina microarrays. k) Representative Oil-Red-O lipid staining in differentiated SGBS human adipocytes following lentiviral knock-down of COBLL1 (shCOBLL1) and GRB14 (shGRB14) compared to the empty vector control (shEV). I) GPDH metabolic activity measurement in shCOBLL1, shGRB14 and shEV differentiated SGBS adipocytes (one-way ANOVA with Tukey's HSD test). m) Basal and insulin-stimulated 3H-2-deoxyglucose uptake in shCOBLL1, shGRB14 and shEV differentiated SGBS adipocytes (one-way ANOVA with Tukey's HISD) test). n) qPCR-based GLUT4 gene expression measurement in shCOBLL1, shGRB14 and shEV differentiated SGBS adipocytes (one-way ANOVA with Tukey's HISD test). o) Actin and COBLL1 staining in siCOBLL1 compared to siNT differentiated human visceral adipocytes at day 14 using phalloidin and COBLL1-antibody (specification: HPA053344) as first and Alexa-Fluor 488 as second antibody. Hoechst staining was used to identify single cell nuclei, magnification x63/oil.

FIG. 24A-24I—a-c) Differences in morphological profiles between TT (n=7) and CC (n=6) allele carriers at day 0 (a), day 3 (b) and day 8 (c) in subcutaneous AMSCs (multi-way ANOVA, significance level 5% FDR). d-f) Differences in morphological profiles between TT (n=7) and CC (n=6) allele carriers at (d) day 0, (c) day 3 and (f) day 8 in visceral AMSCs (multi-way ANOVA, significance level 5% FDR). g) Pie chart illustrating non-redundant differential features per channel and class of measurement at day 8 of subcutaneous adipocyte differentiation in rs6712003 homozygous risk compared to non-risk carriers. h-i) Differences in morphological profiles between AMSCs from COBLL1 KO mice (n=3) and WT (n=4) at (h) day 0 (i) day 2 (t-test, significance level 5% FDR).

FIG. 25A-25B—Generation of Cobll1 mutant mice using CRISPR/Cas9 editing. a) Overview of the CRISPR/Cas9 strategy to delete ˜20 kb of the Cobll1 gene. The gRNA-targeting sequences (gRNAs) are underlined, and the PAM sequences are indicated in bold. Exons are represented as thick black boxes, introns are indicated as black lines with arrows, and the yellow boxes indicate the DNA-targeting region. Red hexagon indicates a stop codon generating a Cobll1 truncated protein. Agarose gel showing the PCR products generated from DNA containing successfully targeted Cobll1 from F0 mouse tail genomic DNA. The 308 bp band corresponds to the genomic deletion. b) A real-time quantitative PCR of levels of Cobll1 mRNA in white adipose tissue (WAT), liver and kidney of Cobll1 WT, (+/−) and (−/−) animals to confirm the Cobll1 ablation in knockout animals. Each group was analyzed using 5 different mice and the values were expressed as the mean±s.e.m and P values by Student's t-test.

FIG. 26—Diagram depicting experimental methodology for determining the association of in vivo, in vitro, and clinical characteristics.

FIG. 27—Trinity association analyses. Diagram showing the association of in vivo (histology), in vitro (LipocyteProfiler), and clinical characteristics. Every arrow indicates a set of analyses and points towards a dataset with variables that can be estimated.

FIG. 28—Clinical characteristics including demographic variables and type 2 diabetes (T2D)) can be introduced to be used with imaging traits.

FIG. 29—In-vivo traits. Features used to represent histology images.

FIG. 30—In-vivo traits. Features used to represent histology images.

FIG. 31—In-vitro cellular traits. Features used to represent Adipocyte Profiler images.

FIG. 32A-32B—In-vitro cellular traits. Features used to represent LipocyteProfiler images.

FIG. 33A-33B—Association between in-vivo traits and clinical characteristics. Using histology-derived size estimates to model clinical characteristics.

FIG. 34A-34B—Association between in-vivo traits and clinical characteristics. Using histology-derived size estimates to model clinical characteristics.

FIG. 35A-35B—Association between in-vivo traits and clinical characteristics. Using histology-derived size estimates to model clinical characteristics.

FIG. 36A-36B—Association between in-vitro traits and clinical characteristics. Using LipocyteProfiler traits to model clinical characteristics (stratified on differentiation time points).

FIG. 37—Association between in-vitro traits and clinical characteristics. Using LipocyteProfiler traits to model clinical characteristics (stratified on differentiation time points).

FIG. 38A-38B—Association between in-vivo and in-vitro traits. Using LipocyteProfiler derived cellular traits to model histology-derived size estimates.

FIG. 39—Association between in-vivo and in-vitro traits. Using LipocyteProfiler derived cellular traits to model histology-derived size estimates.

FIG. 40—Association between in-vivo and in-vitro traits. Using LipocyteProfiler derived cellular traits to model histology-derived size estimates.

FIG. 41A-41B—Association between in-vivo and in-vitro traits. Using LipocyteProfiler derived cellular traits to model histology-derived size estimates.

FIG. 42A-42F—Rs12454712 is associated with a lipodystrophy-like phenotype and is predicted to regulate target genes in adipose tissue and muscle. a) Phenome-wide association study (PheWAS) (Taliun et al. 2020) for rs12454712 shows associations with a number of metabolic traits, including insulin sensitivity, BMI, BMI-adjusted T2D, T2D, BMI-adjusted waist-to-hip ratio (WHIRadjBMI) and WHR b) The 18q21.33 locus contains no other variants in high linkage disequilibrium with the lead variant rs12454712 and overlaps active regulatory marks in adipose tissue and skeletal muscle. Image visualized using the T2D knowledge portal c) Promoter Capture Hi-C and ABC d) eQTL starnet browser e) Schematic description of RNA-seq profiling in patient-derived subcutaneous and visceral adipose-derived mesenchymal stem cells (AMSCs) throughout adipogenesis (day 0, day 3, day 8, day 14) f) Gene expression of target genes in AMSCs at day 0.

FIG. 43A-43K—Allele-specific effect of rs12454712 on ROS and apoptosis in subcutaneous adipocytes. a) LipocyteProfiler was performed in subcutaneous and visceral AMSCs of 11 risk and 5 non-risk haplotype carriers for rs12454712 at four time points during adipocyte differentiation (day 0, 3, 8 and 14) b) In subcutaneous AMSCs, Mito features were different between haplotypes at day 8 and day 14 of differentiation, respectively c) (first panel) Representative images of subcutaneous AMSCs from TT risk (top) and CC non-risk (bottom) haplotype at day 8 of differentiation stained using LipocytePainting. Scale bar=10 μm. (second and third panel) Significant features different between haplotypes at day 8. d) (first panel) Representative images of subcutaneous AMSCs from TT risk (top) and CC non-risk (bottom) haplotype at day 14 of differentiation stained using LipocytePainting. Scale bar=10 μm (second and third panel) Significant features different between haplotypes at day 14. c) BCL2 was silenced in subcutaneous AMSCs using siRNA from five normal-weight female individuals for assessment of cell number, cell morphology throughout differentiation using LipocyteProfiler and mitochondrial respiration using the Seahorse Bioflux Analyser at day 14 of differentiation. AMSCs were treated with siBCL2 for 3 days before induction, at which point knockdown efficiency was ˜60% and maintained until terminal differentiation. f) Predicted ROS levels in subcutaneous AMSCs of risk and non-risk haplotype carriers for rs12454712 at four time points during adipocyte differentiation (day 0, 3, 8 and 14). g) LipocyteProfiles of BCL2-KD and non-targeting control AMSCs (n=5) show differences in mitochondrial and lipid-related features at day 14 of adipocyte differentiation. h) Differential gene expression of BCL2-KD and non-targeting control AMSCs show differences in pro-apoptotic genes and lipid-related genes at day 14 of adipocyte differentiation. i) At day 14, BCL2-KD reduced cell numbers in subcutaneous AMSCs (n=3) by ˜50% as assessed using Hoechst intensity. j) Predicted ROS levels in BCL-KD and control AMSCs at day 14 of differentiation. k) LipocyteProfiles of BCL2-KD and non-targeting control compared to subcutaneous AMSCs from TT risk and CC non-risk haplotypes.

FIG. 44A-44E—Allele-specific effect of rs12454712 on thermogenesis and lipid accumulation in visceral adipocytes. a) LipocyteProfiler in visceral AMSCs of 11 risk and 5 non-risk haplotype carriers for rs12454712 at four time points during adipocyte differentiation (day 0, 3, 8 and 14) revealed a genotype-driven effect specifically on day 14 on mitochondrial and lipid-related features b) Representative images of visceral AMSCs from TT risk and CC non-risk haplotype at day 14 of differentiation stained using LipocytePainting. Scale bar 10 μm. Significant features different between haplotypes at day 14 (graphs). c) VPS4B gene expression (velocity) at day 0 in visceral AMSCs compared to RNA-seq and LipocyteProfile at Day 14. d) Experimental design for comparing isoproterenol treatment to risk and non-risk haplotypes in visceral AMSCs. e) Experiment showing brown adipocyte following FFA treatment in risk and non-risk haplotypes.

FIG. 45A-45D—Polygenic risk for WHRadjBMI manifests in an apoptotic cellular profile in subcutaneous adipocytes. a) Schematic showing subjects in the top and bottom 25% of polygenic risk for WHRadjBMI. b) Representative images of subcutaneous AMSCs from low risk and high risk subjects at day 14 of differentiation stained using LipocytePainting. c) Differences of LipocyteProfiles between top and bottom 25% of polygenic risk for WHRadjBMI were mostly mitochondrial and lipid-related. d) Differences of indicated features between top and bottom 25% of polygenic risk for WHIRadjBMI.

FIG. 46A-46E—Regulatory landscape around rs12454712. a) Phenome-wide association study (PheWAS) using UKBB data visualized in the browser big.stats.ox.ac.uk/ (Elliott et al. 2018) for rs12454712 shows associations with a number of obesity- and fat-related (e.g. Hip circumference) as well as muscle-related (e.g. Basal metabolic rate) traits. b) Hi-C in MSCs (Dixon et al. 2015) visualized using the 3D) Genome browser (Wang et al. 2018). c) rs12454712 lies within an active regulatory element assessed by overlapping the locus with chromatin state maps across 833 reference epigenomes (Boix et al. 2021). d) Activity-by-contact (ABC) target gene prediction (Fulco et al. 2019) in adipocyte nuclei (ENCODE Project Consortium 2004; Zhou et al. 2015) and adipocytes differentiated from adipose-derived mesenchymal stem cells (Schmidt et al. 2015) e) Gene expression of target genes at days 3, 8 and 14 in subcutaneous and visceral adipose-derived mesenchymal stem cells.

FIG. 47A-47C—a) Gene expression compared to LipocyteProfile features. Features having a FDR less than or equal to 5% are shown. b) Effect size of significant Mito features different between CC and TT alleles for rs12454712. c) Effect size of significant Lipid, AGP, and DNA features different between CC and TT alleles for rs12454712.

FIG. 48A-48G—BCL2-KO affects mitochondrial structure and function. a) siBCL2 KO efficiencies b) LipocyteProfiles of BCL2-KD and non-targeting control AMSCs (n=5) at day 0, 3 and 8 of adipocyte differentiation. c) Relative Hoechst staining of BCL2-KD and non-targeting control. d) BCL-2KD increases mitochondrial texture and intensity, resembling the TT risk haplotype e) Representative images of BCL2-KD and non-targeting control. f) Mitochondrial granularity features were increased in BCL2-KO adipocytes specifically for the smaller measurements (Cytoplasm_Granularity_Mito), and decreased for the larger measurements (Cytoplasm_Granularity_Mito), indicating more fragmented mitochondria in BCL2-KO adipocytes compared to siNT-treated cells. Gene expression of hFis, a mitochondrial fission gene, correlated negatively larger granularity measures across adipocytes from 26 individuals. Gene expression of MFN2, a mitochondrial fusion gene, correlated negatively for smaller and positively with larger granularity measures, suggesting that mitochondrial fragmentation phenotype observed in adipocytes from TT risk allele carriers and BCL2-KO adipocytes is indicative of increased mitochondrial g) Mitochondrial stress test using the Seahorse Bioflux Analyser in BCL-KD and control AMSCs (n=3) at day 14 of differentiation shows increased maximal OCR (top) in BCL2-KO AMSCs. A combined measure of OCR and ECR (a measure of extracellular acidification), revealed that BCL2-KD resulted in a more energetic profile compared to control.

FIG. 49A-49C—a) LipocyteProfiler in visceral AMSCs of 11 risk and 5 non-risk haplotype carriers for rs12454712. b) VPS4B gene expression (velocity) at day 0 in visceral AMSCs compared to LipocyteProfile at Day 14. c) LipocyteProfiler comparison of isoproterenol treatment to risk and non-risk haplotypes in visceral AMSCs.

FIG. 50—Diagram showing differences between the TT risk allele and CC non-risk allele.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand and are usually inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minis satellites.

As used herein, the term “type 2 diabetes”, also known as type 2 diabetes mellitus, and often referred to as diabetes includes, e.g., adult-onset diabetes.

There are multiple terms for stem cells derived from adipose tissue, for example, preadipocytes, adipose-derived stromal cells (ADSC), processed lipoaspirated cells, adipose-derived mesenchymal stem cells (AMSC), adipose-derived adult stem cells. (Tsuji W, Rubin J P, Marra K G. Adipose-derived stem cells: Implications in tissue regeneration. World J Stem Cells. 2014; 6 (3): 312-321). These terms are used interchangeably throughout the specification. As used herein, “adipocyte progenitors” can refer to stem cells or any cell intermediates that differentiate into adipocytes.

As used in this context, to “treat” means to cure, ameliorate, stabilize, prevent, or reduce the severity of at least one symptom or a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “in need of treatment” as used herein refers to a judgment made by a caregiver (e.g., physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compositions and therapeutic agents described herein. In embodiments, the judgment by the caregiver has been made, and the subject identified as requiring or benefitting from treatment.

The administration of compositions, agents, cells, or populations of cells, as disclosed herein may be carried out in any convenient manner including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

Reference is made to the manuscript posted Jul. 19, 2021 on BioRxiv and entitled, “Discovering cellular programs of intrinsic and extrinsic drivers of metabolic traits using LipocyteProfiler” and having as authors Samantha Laber, Sophie Strobel, Josep-Maria Mercader, Hesam Dashti, Alina Ainbinder, Julius Honecker, Garrett Garborcauskas, David R. Stirling, Aaron Leong, Katherine Figueroa, Nasa Sinnott-Armstrong, Maria Kost-Alimova, Giacomo Deodato, Alycen Harney, Gregory P. Way, Alham Saadat, Sierra Harken, Saskia Reibe-Pal, Hannah Ebert, Yixin Zhang, Virtu Calabuig-Navarro, Elizabeth McGonagle, Adam Stefek, Josée Dupuis, Beth A. Cimini, Hans Hauner, Miriam S. Udler, Anne E. Carpenter, Jose C. Florez, Cecilia M. Lindgren, Suzanne B. R. Jacobs, Melina Claussnitzer (bioRxiv 2021.07.17.452050; doi: doi.org/10.1101/2021.07.17.452050).

All publications, published patent documents, and patent applications cited herein are hereby incorporated by reference to the same extent as though each individual publication, published patent document, or patent application was specifically and individually indicated as being incorporated by reference.

### Overview

Most disease-associated genetic loci map to more than one disease or trait, suggesting they act through multiple cell types and tissues giving rise to complex disease phenotypes. This pervasive pleiotropy of human diseases presents a tremendous burden on identifying mediating mechanisms and therapeutic targets. Multiple metabolic risk haplotypes are associated with risk for metabolic diseases. However, whether a haplotype actually causes a disease and the mechanisms that cause the disease are unknown. For example, a risk haplotype may be important for disease in a specific cell type at a specific time. Integration of phenotypic and transcriptional profiling in primary human cells allows for functional characterization of disease-associated genetic variants. Applicants have analyzed multiple risk haplotypes and determined the function of risk haplotypes involved in causation of specific metabolic phenotypes, such as type 2 diabetes and lipodystrophy.

The metabolic risk haplotype at 2q24.3 displays cross-phenotype association signatures that are reminiscent of the MONW/MOH phenotype and associate with increased risk of T2D, increased HOMA-IR, increased WHR adjusted for BMI (WHRadjBMI) and decreased body fat percentage, decreased estimated subcutaneous adipose tissue mass, and cardiometabolic trait risk (Kooner et al. 2011; DIAbetes Genetics Replication And Met . . . ; Morris et al. 2012; Heid et al. 2010; Lu et al. 2016). Consistent with these associations, the 2q24.3 locus falls into the lipodystrophy cluster of T2D loci (Udler et al. 2018), suggesting adipocytes as the mediating cell type at this locus. Notably, amongst the 20 loci identified in the T2D lipodystrophy process-specific cluster (Udler et al. 2018), the 2q24.3 locus is the top scoring one, inferring the strongest contribution to a ‘lipodystrophic-like’ phenotype amongst the T2D GWAS loci. However, similar to the vast majority of genetic risk loci identified through GWAS, the function of the 2q24.3 metabolic risk locus is currently unknown.

Applicants have identified causal variants leading to reduced COBL11 expression. Applicants further demonstrate that the cellular program under the genetic control of the 2q23.4 risk locus and the effector gene COBL11 is characterized by an impairment of actin cytoskeleton remodeling processes in differentiating subcutaneous adipocytes and a subsequent failure of these cells to accumulate lipids, and develop into a metabolically active and insulin-sensitive subcutaneous adipocyte. While not being bound by a particular scientific theory, individual risk for T2D and fasting insulin is believed to be modified by changes to the mass, distribution, and function of adipose tissue (Lotta et al 2017; Small et al 2018), and that a metabolically healthy state is largely dependent on subcutaneous adipose tissue expandability. As disclosed in further detail herein. Applicants have, for the first time, identified actin cytoskeleton remodeling as a critical factor for subcutaneous adipocyte function and as causally involved in metabolic disease progression in humans, thus identifying COBL11 and causal variants impacting COBL11 expression or function as viable therapeutic targets for treating and/or preventing T2D.

Using an unbiased approach based on phenotypic profiling of primary human adipocytes Applicants dissected the function of a genomic risk locus in 18q21.33 that is strongly associated with a lipodystrophy-like metabolic phenotype. Applicants showed that the haplotype modifies gene expression of at least three target genes (BCL2, KDSR, and VPS4B) in at least three diabetes-related tissues (subcutaneous adipose tissue, visceral adipose tissue, and skeletal muscle) during specific temporal windows with distinct cellular and morphological consequences that converge to modulate disease susceptibility. BCL2 and KDSR showed reduced expression in subcutaneous adipose-derived mesenchymal stem cells (AMSCs), however, reduced apoptosis and mitochondrial morphological features were observed in mature adipocytes that are terminally differentiated. BCL2 also showed reduced expression in skeletal muscle. VSPB4 showed increased expression in visceral adipose-derived mesenchymal stem cells (AMSCs), however, mitochondrial morphological features were observed in mature adipocytes that are terminally differentiated. The genotype mediated expression on target gene expression was observed in AMSCs and the morphological features were observed in differentiated adipocytes. Applicants identified that the rs12454712 variant increases apoptosis and apoptosis related genes in adipocytes. Thus, inhibiting apoptosis can be used to treat metabolic diseases caused by this mechanism.

Specifically, phenotype-informed clustering of T2D identified a subset of T2D loci that follow clinical presentation of insulin resistance with a “lipodystrophy-like” fat distribution (low BMI, adiponectin, and high-density lipoprotein cholesterol, and high triglycerides) (Udler et al. 2018). Amongst those genetic signals was rs12454712 on 18q21.33, a genetic locus of unknown function that maps to the first intron of the BCL2 gene. The 18q21.33 locus, like most genetic risk loci, lies within the non-coding genome, making the identification of mediating target genes and mechanisms challenging and experimentally intense. Non-coding variants may regulate one or more genes across long genomic distances, and the same variant might have very context-specific functions, including regulating different genes in different cell types under specific environmental conditions. In this study, we set out to decipher the function of the 18q21.33 metabolic risk locus. By combining novel experimental and statistical methods, Applicants mechanistically dissect this pleiotropic locus into mediating cell types and target genes, developmental time-points of action and cellular functions that could account for the associated phenotypes in humans.

Together, the findings highlight the complexities underlying disease-associated loci in humans and showcase an approach of unbiased dissection of mediating mechanisms.

Accordingly, embodiments disclosed herein are directed to methods for treating subjects at risk for, or suffering from, Type-2 Diabetes (T2D) or lipodystrophy. A subject may be at risk for T2D if they clinically demonstrate increased glucose tolerance, increased insulin resistance, are identified as possessing a MONW/MOH risk loci or lipodystrophy risk loci, or a combination thereof. Thus, treatment methods disclosed herein are directed to subjects who are both at risk for T2D) or lipodystrophy or have been diagnosed with T2D) or lipodystrophy. In example embodiments, the methods provide treatment options for individuals who possess certain metabolic risk loci, in particular those who classify as MONW/MOH. In one aspect, embodiments disclosed herein are directed to methods of treating subjects at risk for, or suffering from T2D, by administering one or more agents that increase COBL11 expression or COBL11 activity in adipocyte or adipocyte-progenitor cell types. In another aspect, embodiments disclosed herein are directed to methods of treating subjects at risk for, or suffering from, T2D by administering one or more agents that can edit causal risk variants in adipocyte or adipocyte-progenitors to a wild-type or non-risk variant. In certain example embodiments, the causal risk variant is an intronic variant in the COBL11 gene. In certain example embodiments, the intronic variant alters the binding affinity of POU Class 2 Homeobox 2 (POU2F2) to an enhancer controlling COBL11 expression. In certain example embodiments, the causal variant includes rs6712203. In another aspect, embodiments disclosed herein are directed to methods of treating subjects at risk for, or suffering from, lipodystrophy by administering one or more apoptosis inhibitors. In another aspect, embodiments disclosed herein are directed to methods of treating subjects at risk for, or suffering from, lipodystrophy by administering one or more agents that can edit causal risk variants in adipocyte-progenitors to a wild-type or non-risk variant. In certain example embodiments, the causal variant includes rs12454712.

In another aspect, embodiments disclosed herein are directed to a method for a identifying the presence of a rs6712203 or rs12454712 variant in a subject by conducting a genotyping assay on a biological sample from the subject. In one example embodiment, identification of the rs6712203 variant further comprises treating the subject with one or more agent that increases the expression or activity of COBLL1, or enhances actin remodeling; corrects the one or more variants to a wild type sequence with a gene editing system; or adoptive cell transfer comprising allogenic adipocyte donor exhibiting wild type COBLL1 expression, or autologous adipocyte donors genetically modified to correct the one or more variants to a wild type sequence. In one example embodiment, identification of the rs12454712 variant further comprises treating the subject with one or more agent that increases the expression or activity of BCL2, or inhibits apoptosis; corrects the one or more variants to a wild type sequence with a gene editing system; or adoptive cell transfer comprising allogenic adipocyte donor exhibiting wild type expression, or autologous adipocyte donors genetically modified to correct the one or more variants to a wild type sequence.

In another aspect, embodiments disclosed herein are directed to a method of treating a person at risk for, or suffering from T2D, based on detecting one or more polygenic risk indicators, and administering one or more treatments for increasing the expression of activity of COBLL1, or that enhance actin remodeling in adipocyte or adipocyte progenitors, if the polygenic risk indicator is detected, or treating the subject with a T2D standard-or-care therapy if the polygenic risk indicator is not detected.

In another aspect, embodiments disclosed herein are directed to methods for unbiased high-throughput multiplex profiling of morphological and cell phenotypes simultaneously. At least four fluorescent dyes may be used to stain cells. The stained cells are imaged using an automated image analysis pipeline, and morphological and cellular phenotypes are identified from the resulting images.

### Methods of Treatment

In one example embodiment, a method of treating subjects that are at risk for, or suffering from Type-2 Diabetes (T2D)), comprises administering to a subject in need thereof, a therapeutically effective amount of one or more agents that increase the expression or activity of COBLL1, or that enhance actin remodeling, in adipocytes or adipocyte progenitors. In one example embodiment, the subject may suffer from a cellular dysfunction that leads to impairment of actin cytoskeleton remodeling in adipocytes and/or adipocyte progenitors. In another example embodiment, the subject may have one or more MONW/MOH risk loci.

In another example embodiment, a method of treating subjects that are at risk for, or suffering from lipodystrophy, comprises administering to a subject in need thereof, a therapeutically effective amount of one or more agents that increase the expression or activity of BCL2 or KDSR, decrease the expression or activity of VPS4B, or that inhibit apoptosis, in adipocytes or adipocyte progenitors. In one example embodiment, the subject may suffer from a cellular dysfunction that leads to impairment of mitochondrial mechanisms that prevent apoptosis in adipocytes. In another example embodiment, the subject may have one or more lipodystrophy risk loci.

As used herein “lipodystrophy” refers to a group of genetic or acquired disorders in which the body is unable to produce and maintain healthy fat tissue. The medical condition is characterized by abnormal or degenerative conditions of the body's adipose tissue. (“Lipo” is Greek for “fat”, and “dystrophy” is Greek for “abnormal or degenerative condition”.) This condition is also characterized by a lack of circulating leptin which may lead to osteosclerosis. The absence of fat tissue is associated with insulin resistance, hypertriglyceridemia, non-alcoholic fatty liver disease (NAFLD) and metabolic syndrome. Due to an insufficient capacity of subcutaneous adipose tissue to store fat, fat is deposited in non-adipose tissue (lipotoxicity), leading to insulin resistance. Patients display hypertriglyceridemia, severe fatty liver disease and little or no adipose tissue. Average patient lifespan is approximately 30 years before death, with liver failure being the usual cause of death. In contrast to the high levels seen in non-alcoholic fatty liver disease associated with obesity, leptin levels are very low in lipodystrophy. In certain embodiments, polygenic lipodystrophy includes insulin resistance with a “lipodystrophy-like” fat distribution, insulin sensitivity, BMI-adjusted T2D, increased BMI-adjusted waist-to-hip ratio (WHRadjBMI), and/or Type-2 Diabetes (T2D).

### Small Molecules Targeting COBLL1, BCL2, KDSR, or VPS4B Expression or Activity

In certain example embodiments, a method of treating subjects that are at risk for, or are suffering from Type 2 Diabetes (T2D) comprises administering one or more small molecules that increases expression of COBLL1, increases binding of POU2F2 to a binding site in an enhancer regulating COBLL1 expression, or that enhances actin remodeling in adipocytes or adipocyte progenitors.

In certain example embodiments, a method of treating subjects that are at risk for, or are suffering from lipodystrophy comprises administering one or more small molecules that increases expression of BCL2 in pre-adipocytes (e.g., subcutaneous AMSCs) and/or skeletal muscle, increases binding of BCL2 to pro-apoptotic proteins, or that inhibits apoptosis in adipocytes.

In certain example embodiments, a method of treating subjects that are at risk for, or are suffering from lipodystrophy comprises administering one or more small molecules that increases expression of KDSR in pre-adipocytes (e.g., subcutaneous AMSCs), increases activity of KDSR, or that enhances mitochondrial function in adipocytes.

In certain example embodiments, a method of treating subjects that are at risk for, or are suffering from lipodystrophy comprises administering one or more small molecules that increases expression of VPS4B in pre-adipocytes (e.g., visceral AMSCs), increases activity of VPS4B, or that enhances mitochondrial in adipocytes.

The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In example embodiments, the small molecule may act as an antagonist or agonist.

### Small Molecules That Enhance Actin Remodeling in Adipocyte or Adipocyte Progenitors

In one example embodiment, a method for treating subjects suffering from, or at risk of, T2D comprises administering small molecules that target a similar mechanism of action as COBLL1, that is enhancing actin remodeling in adipocytes or adipocyte progenitors.

Actin is a protein and invertebrates have three main monomer isoforms including α-isoforms of skeletal, cardiac, and smooth muscles; β-isoforms in non-muscle and muscle cells; and γ-isoforms in non-muscle and muscle cells. Actin participates in protein-protein interactions and can transition between monomeric states called G-actin and filamentous states called F-actin. Actin plays a role in many cellular functions such as cell motility, cell shape, polarity, and regulation of transcription. Actin belongs to a structural superfamily with sugar kinases, hexokinases, and Hsp70 proteins. Actin comprises of around 375 amino acids and folds into two major a/B domains or inner and outer domains further comprising of four subdomains.

The actin cytoskeleton comprises of a network of fibrous actin and is the system that allows organelle, chromosome, and cell movement. It is also the structural support for a cell and can change the cell morphology by assembling or disassembling. This reorganization is also called actin remodeling and is controlled by actin-binding proteins that regulate nucleation, branching, elongation, bundling, severing, and capping of actin filaments.

Herein, improper actin cytoskeleton remodeling is implicated in metabolic disease progression. It has been shown that adipocyte size is positively correlated with impaired insulin sensitivity and glucose tolerance. Moreover, adipocyte size was shown to predict Type-2 diabetes. (Hansson, B., et al. Adipose cell size changes are associated with a drastic actin remodeling. Sci. Rep. 9, 12941, 2019). As a major structural modifier in adipocytes, actin cytoskeleton remodeling can be regulated as a treatment method for preventing or treating metabolic disorders or diseases. Further, the actin cytoskeleton remodeling process is required for differentiating subcutaneous adipocytes, and subsequent accumulation of lipids and development into metabolically active and insulin-sensitive subcutaneous adipocytes. Treatment may be regulation of COBLL1 expression, regulation of POU2F2 binding, and/or modification of a rs6712203 genetic variant.

In one example embodiment, actin remodeling can be enhanced by an agent selected from the group consisting of geodiamolides (Geodiamolide H), Jasplakinolide, Chondramide (Chondramide A), ADF/Cofilin, Arp2/3 complex, Profilin, Gelsolin (Flightless-I), Formin, Villin (Advillin), and Adseverin. In another example embodiment, the agent is a geodiamolide which is a cyclodepsipeptide commonly derived from marine sponges. In specific non-limiting embodiments, the geodiamolide is Geodiamolide H. In another example embodiment, the agent is a jasplakinolide, also known as jaspamide, is a cyclic peptide with a fifteen-carbon macrocyclic ring containing three amino acid residues 1-alanine, N-methyl-2-bromotryptophan, and βtyrosine. In another example embodiment, the agent is chondramide, which is a cyclodepsipeptide isolated from the mycobacterium Chondromyces crocatus. In another example embodiment, the agent is ADF/cofilin, which are actin-binding proteins of the actin-depolymerization factor family. ADF may also be known as destrin. In some embodiments, the agent is Arp2/3 complex, which is an assembly of seven protein subunits. Two of the seven subunits are actin-related proteins ARP2 and ARP3. In another example embodiment, the agent is profilin, which is an actin-binding protein. In another example embodiment, the agent is gelsolin, which is an actin binding/regulatory protein. In specific non-limiting embodiments, the gelsolin is Flightless I. In another example embodiment, the agent is formin, which is a protein with a conserved FH2 domain that stabilizes actin. In certain example embodiments, the agent is vilin which is a calcium-regulated actin-binding protein. In specific non-limiting embodiments, the vilin is advilin, a member of a gelsolin/villin superfamily of actin binding and regulatory proteins. In another example embodiment, the agent is adseverin also known as scinderin, which belongs to the gelsolin superfamily and is an actin severing and capping protein.

Small Molecules that Inhibit Apoptosis or Target BCL2 Expression

In one example embodiment, a method for treating subjects suffering from, or at risk of, lipodystrophy comprises administering small molecules that inhibit apoptosis or enhance BCL2 expression in adipocytes or adipocyte progenitors (e.g., BCL2). In one example embodiment, apoptosis can be inhibited by an agent selected from the group consisting of Ginkgo biloba extract (FGb 761), Rhodiola crenulata extract (RCF), salidroside, dehydroepiandrosterone, allopregnanolone, diosmin, glycine, M50054, BI-6C9, TC9-305 (2-sulfonyl-pyrimidinyl derivatives), BI-11A7, 3-o-tolylthiazolidine-2,4-dione, minocycline, methazolamide, melatonin, gamma-tocotrienol (GTT), 3-hydroxypropyl-triphenylphosphonium (TPP)-conjugated imidazole-substituted oleic acid (TPP-IOA), TPP-conjugated stearic acid (TPP-ISA), TPP-6-ISA, CLZ-8, Xanthan gum (XG), PD98059, Vitamin E, and Tanshinone (see, e.g., El-Shimaa Mohamed Naguib Abdelhafcz, Sara Mohamed Naguib Abdelhafez Ali, Mohamed Ramadan Eisa Hassan and Adel Mohammed Abdel-Hakem (June 20th 2019). Apoptotic Inhibitors as Therapeutic Targets for Cell Survival, Cytotoxicity-Definition, Identification, and Cytotoxic Compounds, Erman Salih Istifli and Hasan Basri Ila, IntechOpen, DOI: 10.5772/intechopen.85465).

Rhodiola crenulata extract (RCE) is an edible alcohol extract, conserving greatly the mitochondrial integrity and in turn prohibiting the release of cytochrome C, which leads to cell death. The effective concentration of the most important component, salidroside, was ˜4% (w/w). Glycine can upregulate of Bcl2 and Bcl2-bax (apoptosis regulator BAX). Minocycline directly inhibits the release of cytochrome C from mitochondria. Methazolamide was FDA approved for the treatment of glaucoma, while melatonin inhibited oxygen/glucose deprivation induced cell death, loss of mitochondrial membrane potential, release of mitochondrial factors, pro-IL-1β processing, and activation of caspase-1 and -3. Gamma-tocotrienol (GTT) prevents the activation of caspase-3 and caspase-9, reducing the release of cytochrome C from the mitochondria and preventing H2O2-induced apoptosis. 3-hydroxypropyl-triphenylphosphonium (TPP)-conjugated imidazole-substituted oleic acid (TPP-IOA) and stearic acid (TPP-ISA) exert strong specific liganding of heme-iron in cytochrome C/cardiolipin (CI) complex and effectively suppress its peroxidase activity and CL peroxidation, thus preventing cytochrome C release and cell death. TPP-6-ISA is an effective inhibitor of the peroxidase function of cyt c/CL complexes with a significant antiapoptotic activity. CLZ-8 is capable of targeting a PUMA protein and provides for apoptosis resistance. Xanthan gum (XG) is an extracellular polysaccharide secreted by microorganisms that decreases the apoptosis of chondrocytes, downregulates the expressions of active caspase-9, active caspase-3 and bax, and upregulates the expression of bcl-2. PD98059 inhibits apoptosis through inhibition of BAX and other factors. Vitamin E can modify BAX and BCL-2 expression levels. Tanshinone can inhibit the expression of Bax and stimulate the expression of Bcl-2.

### Gene Therapy Approaches for Increasing COBLL1 Expression

In one example embodiment, subjects at risk for, or suffering T2D, are treated by increasing expression of COBLL1 using a gene therapy approach. As used herein, the terms “gene therapy”, “gene delivery”, “gene transfer” and “genetic modification” are used interchangeably and refer to modifying or manipulating the expression of a gene to alter the biological properties of living cells for therapeutic use.

In one example embodiment, a vector for use in gene therapy comprises a sequence encoding COBLL1 or a functional fragment thereof, and is used to deliver said sequence to adipocyte or adipocyte progenitors to increase expression of COBLL1 in those cells types. The vector may further comprise one or more regulatory elements to control expression of COBLL1. The vector may further comprise regulatory/control elements, e.g., promoters, enhancers, introns, polyadenylation signals, Kozak consensus sequences, or internal ribosome entry sites (IRES). The vector may further comprise cellular localization signals, such as a nuclear localization signal (NLS) or nuclear export signal (NES). The vector may further comprise a targeting moiety that directs the vector specifically to adipocyte or adipocyte progenitors. In another example embodiment, the vector may comprise a viral vector with a trophism specific for adipocyte and adipocyte progenitors.

### COBLL1 Sequence

COBLL1, also known as CORDON-BLEU WHI2 REPEAT PROTEIN-LIKE 1; CORDON-BLEU PROTEIN-LIKE 1; COBL-LIKE 1; COBLR1; and KIAA0977, is located on the human 2Q24.3 locus. In one example embodiment, the polynucleotide sequence included in the vector is a DNA sequence derived from the primary accession number Q53SF7. In another example embodiment, the DNA sequence is Q53SF7. In another example embodiment, the DNA sequence is derived from the secondary accession numbers A6NM73, Q6IQ33, Q7Z316, Q9BRH4, Q9UG88, and Q9Y213. In another example embodiment, the DNA sequence is selected from the group consisting of A6NMZ3, Q6IQ33, Q7Z316, Q9BRH4, Q9UG88, and Q9Y2I3.

In another example embodiment, the polynucleotide sequence included in the vector is a RNA sequence derived from; NM_001365672; NM_014900; NM_001278458; NM_001278460; NM_001278461; NM_001365670; NM_001365671; NM_001365673; NM_001365674; or NM_001365675. In another example embodiment, the polynucleotide sequence included in the vector is a RNA sequence selected from the group consisting of: NM_001365672; NM_014900; NM_001278458; NM_001278460; NM_001278461; NM_001365670; NM_001365671; NM_001365673; NM_001365674; or NM_001365675. In another example embodiment, the sequence include in the vector is derived from mRNA selected from the group consisting of: AB023194.1; AI261693.1; AK001813.1; AK002054.1; AK002057.1; AK075181.1; AK225849.1; AK294937.1; AL049939.1; AL832824.1; BC006264.2; BC071588.1; BX537877.1; BX648994.1; BX649112.1; or CB989062.1. In another example embodiment, the sequence included in the vector is a mRNA sequence selected from the group consisting of: AB023194.1; AI261693.1; AK001813.1; AK002054.1; AK002057.1; AK075181.1; AK225849.1; AK294937.1; AL049939.1; AL832824.1; BC006264.2; BC071588.1; BX537877.1; BX648994.1; BX649112.1; or CB989062.1.

All gene name symbols as used throughout the specification refer to the gene as commonly known in the art. The examples described herein that refer to gene names are to be understood to encompass human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. Reference to a gene encompasses the gene product (e.g., protein encoded for by the gene).

### Regulatory Elements

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operably-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The term “operably linked” as used herein also refers to the functional relationship and position of a promoter sequence relative to a polynucleotide of interest (e.g., a promoter or enhancer is operably linked to a coding sequence if it affects the transcription of that sequence). Typically, an operably linked promoter is contiguous with the sequence of interest. However, enhancers need not be contiguous with the sequence of interest to control its expression. The term “promoter”, as used herein, refers to a nucleic acid fragment that functions to control the transcription of one or more polynucleotides, located upstream of the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. A “tissue-specific” promoter is only active in specific types of differentiated cells or tissues.

In another embodiment, the vector of the invention further comprises expression control sequences including, but not limited to, appropriate transcription sequences (i.e., initiation, termination, promoter, and enhancer), efficient RNA processing signals (e.g., splicing and polyadenylation (polyA) signals), sequences that stabilize cytoplasmic mRNA, sequences that enhance translation efficiency (i.e., Kozak consensus sequence), and sequences that enhance protein stability. A great number of expression control sequences, including promoters which are native, constitutive, inducible, or tissue-specific are known in the art and may be utilized according to the present invention.

In another embodiment, the vector of the invention further comprises a post-transcriptional regulatory region. In a preferred embodiment, the post-transcriptional regulatory region is the Woodchuck Hepatitis Virus post-transcriptional region (WPRE) or functional variants and fragments thereof and the PPT-CTS or functional variants and fragments thereof (see, e.g., Zufferey R, et al., J. Virol. 1999; 73:2886-2892; and Kappes J, et al., WO 2001/044481). In a particular embodiment, the post-transcriptional regulatory region is WPRE. The term “Woodchuck hepatitis virus posttranscriptional regulatory element” or “WPRE”, as used herein, refers to a DNA sequence that, when transcribed, creates a tertiary structure capable of enhancing the expression of a gene (see, e.g., Lec Y, et al., Exp. Physiol. 2005; 90 (1): 33-37 and Donello J, et al, J. Virol. 1998; 72 (6): 5085-5092).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990).

Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as adipose tissue or particular cell types (e.g., adipocytes or adipocyte progenitors). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Also encompassed by the term “regulatory element” are enhancer elements (e.g., adipose specific enhancers or Woodchuck Hepatitis Virus Posttranscriptional Regulatory Element (WPRE)). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., COBLL1).

In a preferred embodiment, the adipose-tissue specific regulatory region according to the invention comprises the adipose-specific aP2 enhancer and the basal aP2 promoter (see, e.g., Rival Y, et al., J. Pharmacol. Exp. Ther. 2004:31 1 (2): 467-475). The region comprising the adipose-specific aP2 enhancer and the basal aP2 promoter is also known as “mini/aP2 regulatory region” and is formed by the basal promoter of the aP2 gene and the adipose-specific enhancer of said aP2 gene. Preferably, the aP2 promoter is murine. (See, e.g., Graves R, et al, Mol. Cell Biol. 1992; 12 (3): 1202-1208; and Ross S, et al, Proc. Natl. Acad. Sci. USA 1990; 87:9590-9594).

In another preferred embodiment, the adipose-tissue specific regulatory region according to the invention comprises the adipose-specific UCP1 enhancer and the basal UCP1 promoter. (Sec, e.g., del Mar Gonzalez-Barroso M, et al, J. Biol. Chem. 2000; 275 (41): 31722-31732; and Rim J, et al, J. Biol. Chem. 2002; 277 (37): 34589-34600). The region comprising the adipose-specific (CPI enhancer and the basal UCP1 promoter is also known as “mini/UCP regulatory region” and refers to a combination of the basal promoter of the UCP1 gene and the adipose-specific enhancer of said UCP1 gene. Preferably, a rat UCP1 promoter is used. (See, e.g., Larose M, et al, J. Biol. Chem. 1996; 271 (49): 31533-31542; and Cassard-Doulcier A, et al, Biochem. J. 1998; 333:243-246).

### Vector Selection

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g., adenoviruses, adeno-associated viruses as well as retroviruses and lentiviruses), as well as non-viral vectors such as plasmids.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably-linked. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, COBLL1 is introduced to adipocytes or adipocyte progenitors by means of an AAV viral vector. The terms “adeno-associated virus”, “AAV virion”, and “AAV particle”, as used interchangeably herein, refer to a virion composed of at least one AAV capsid protein (preferably all capsid proteins of a particular AAV serotype) and an encapsidated polynucleotide AAV genome. If the particle comprises a heterologous polynucleotide flanked by AAV inverted terminal repeats (i.e., a polynucleotide that is not a wild-type AAV genome, e.g., a transgene is delivered to a mammalian cell), it is often referred to as an “AAV vector particle” or “AAV vector”. AAV refers to a virus belonging to the genus dependovirus parvoviridae. The AAV genome is approximately 4.7 kilobases long and consists of single-stranded deoxyribonucleic acid (ssDNA), which can be in either the positive or negative orientation. The genome comprises Inverted Terminal Repeats (ITRs), and two Open Reading Frames (ORFs), at both ends of the DNA strand: rep and cap. The Rep framework is formed by four overlapping genes encoding the Rep proteins required for the AAV life cycle. The cap framework contains overlapping nucleotide sequences of the capsid proteins: VP1, VP2, and VP3, which interact together to form an icosahedral symmetric capsid (see, e.g., Carter B, Adeno-assisted viruses and ado-assisted viruses vectors for genetic drive, Lassic D, et al, eds., “Gene Therapy: Therapeutic Mechanisms and Strategies” (Marcel Dekker, Inc., New York, NY, US, 2000); and Gao G, et al, J. Virol. 2004; 78 (12): 6381-6388). The term “adeno-associated virus ITR” or “AAV ITR” as used herein refers to inverted terminal repeats present at both ends of the DNA strand of the genome of an adeno-associated virus. The ITR sequences are required for efficient proliferation of the AAV genome. Another characteristic of these sequences is their ability to form hairpins. This property contributes to its own priming, which allows synthesis of the second DNA strand independent of the priming enzyme. It has also been shown that ITRs are essential for integration and rescue of wild-type AAV DNA into the host cell genome (i.e., chromosome 19 of humans) and for efficient encapsidation of AAV DNA that binds to the resulting fully assembled, DNase-resistant AAV particles.

The term “AAV vector” as used herein further refers to a vector comprising one or more polynucleotides of interest (or transgenes) flanked by AAV terminal repeats (ITRs). Such AAV vectors can be replicated and packaged as infectious viral particles when present in a host cell that has been transfected with a vector that can encode and express Rep and Cap gene products (i.e., AAV Rep and Cap proteins), and wherein the host cell has been transfected with a vector that encodes and expresses proteins from adenovirus open reading frame F4orf 6. When an AAV vector is incorporated into a larger polynucleotide (e.g., a chromosome or another vector, such as a plasmid for cloning or transfection), then the AAV vector is typically referred to as a “protein-vector”. This protein-vector can be “rescued” by replication and encapsidation in the presence of AAV packaging functions and the necessary helper functions provided by E4orf 6.

In one example embodiment, gene therapy uses an adeno-associated viral (AAV) vector comprising a recombinant viral genome wherein said recombinant viral genome comprises an expression cassette comprising an adipose tissue-specific transcriptional regulatory region operably linked to a polynucleotide encoding for COBLL1 (AAV vectors can also be used for any compositions described herein, such as a programmable nuclease). AAV according to the present invention can include any serotype of the 42 serotypes of AAV known. In another example embodiment, the AAV is as described previously for adipose tissue specific tropism (see, e.g., WO2014020149A1; and Bates R, Huang W, Cao L. Adipose Tissue: An Emerging Target for Adeno-associated Viral Vectors. Mol Ther Methods Clin Dev. 2020; 19:236-249). In particular, the AAV may include an adipocyte specific promoter.

In particular, the AAV of the present invention may belong to the serotype AAV1, AAV2, AAV3 (including types 3A and 3B), AAV4, AAV5, AAV6, AAV7, AAV8, AAV9, AAV10, AAV11 and any other AAV. In a preferred embodiment, the adeno-associated viral vector of the invention is of a serotype selected from the group consisting of the AAV6, AAV7, AAV8, and AAV9 serotypes. In more preferred embodiments, the adeno-associated viral vector of the invention is an AAV8 serotype. In more preferred embodiments, the adeno-associated viral vector of the invention is the engineered hybrid serotype Rec2 (see, e.g., Charbel Issa, et al., 2013, Assessment of tropism and effectiveness of new primate-derived hybrid recombinant AAV serotypes in the mouse and primate retina PLOS ONE, 8 (2013), p. e60361). In one example embodiment, Rec2 can be used for oral administration, as oral administration of Rec2 results in preferential transduction of BAT with absence of transduction in the gastrointestinal track.

The genome of the AAV according to the invention typically comprises the cis-acting 5′ and 3′ inverted terminal repeat sequences and an expression cassette (see, e.g., Tijsser P, Ed., “Handbook of Parvoviruses” (CRC Press, Boca Raton, FL, US, 1990, pp. 155-168)).

The polynucleotide of the invention can comprise ITRs derived from any one of the AAV serotypes. In a preferred embodiment, the ITRs are derived from the AAV2 serotype. The AAV of the invention comprises a capsid from any serotype. In particular embodiment, the capsid is derived from the AAV of the group consisting on AAV1, AAV2, AAV4, AAV5, AAV6, AAV7, AAV8 and AAV9. In a preferred embodiment, the AAV of the invention comprises a capsid derived from the AAV8 or AAV9 serotypes.

In another particular embodiment, the AAV vector is a pseudotyped AAV vector (i.e., the vector comprises sequences or components originating from at least two distinct AAV serotypes). In a particular embodiment, the pseudotyped AAV vector comprises an AAV genome derived from one AAV serotype (e.g., AAV2), and a capsid derived at least in part from a distinct AAV serotype. In a preferred embodiment, the adeno-associated viral vector used in the method for transducing cells in vitro or in vivo has a serotype selected from the group consisting of AAV6, AAV7, AAV8, and AAV9, and the adeno-associated virus ITRs are AAV2 ITRs.

In one example embodiment, adeno-associated viral vectors of the AAV6, AAV7, AAV8, and AAV9 serotypes are capable of transducing adipose tissue cells efficiently. This feature makes possible the development of methods for the treatment of diseases which require or may benefit from the expression of a polynucleotide of interest in adipocytes (e.g., COBLL1). In particular, this finding facilitates the delivery of polypeptides of interest to a subject in need thereof by administering the AAV vectors of the invention to the patient, thus generating adipocytes capable of expressing the polynucleotide of interest and its encoded polypeptide in vivo (e.g., COBLL1).

In one embodiment the AAV vector contains one promoter with the addition of at least one target sequence of at least one miRNA.

In one example embodiment, the transcriptional regulatory region within the AAV comprises a mini/aP2 regulatory region when white adipocytes or stem cells for differentiating to white adipocytes are transduced. In another example embodiment, the transcriptional regulatory region within the AAV comprises a mini/UCP1 regulatory region when brown adipocytes or stem cells for differentiating to brown adipocytes are transduced. In another example embodiment, the transduced cells can be implanted in the human or animal body to obtain the desired therapeutic effect (described further herein in section on ACT). Thus, the invention also relates to a method for the treatment or prevention of a disease which comprises administering to a subject in need thereof the adipocytes or cell compositions obtained according to the method of the invention.

In one example embodiment, COBLL1 is introduced to adipocytes or adipocyte progenitors by means of a lentiviral viral vector (see, e.g., Balkow A, Hoffmann L S, Klepac K, et al. Direct lentivirus injection for fast and efficient gene transfer into brown and beige adipose tissue. J Biol Methods. 2016; 3 (3): e48. Published 2016 Jul. 16. doi: 10.14440/jbm.2016.123). Lentiviruses are enveloped, single stranded RNA viruses that belong to the family of Retroviridae. Moreover, lentiviral vectors are preferred as they are able to transduce or infect non-dividing cells and typically produce high viral titers.

In one example embodiment, the vector is a “plasmid,” which refers to a circular double stranded DNA loop into which additional DNA segments can be inserted, such as by standard molecular cloning techniques.

In one example embodiment, the vector is an mRNA vector (see, e.g., Sahin, U, Kariko, K and Tureci, O (2014). mRNA-based therapeutics-developing a new class of drugs. Nat Rev Drug Discov 13:759-780; Weissman D, Kariko K. mRNA: Fulfilling the Promise of Gene Therapy. Mol Ther. 2015; 23 (9): 1416-1417. doi: 10.1038/mt.2015.138; Kowalski P S, Rudra A, Miao L, Anderson D G. Delivering the Messenger: Advances in Technologies for Therapeutic mRNA Delivery. Mol Ther. 2019; 27 (4): 710-728. doi: 10.1016/j.ymthe.2019.02.012; Magadum A, Kaur K, Zangi L. mRNA-Based Protein Replacement Therapy for the Heart. Mol Ther. 2019; 27 (4): 785-793. doi: 10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7 (5): 319-334. doi: 10.4155/tde-2016-0006; and Khalil A S, Yu X, Umhoefer J M, et al. Single-dose mRNA therapy via biomaterial-mediated sequestration of overexpressed proteins. Sci Adv. 2020; 6 (27): caba2422). In an exemplary embodiment, mRNA encoding for COBL11 is delivered using lipid nanoparticles (see, e.g., Reichmuth, et al., 2016) and administered directly to adipose tissue. In an exemplary embodiment, mRNA encoding for COBLL1 is delivered using biomaterial-mediated sequestration (see, e.g., Khalil, et al., 2020) and administered directly to adipose tissue. Sequences present in mRNA molecules, as described further herein, are applicable to mRNA vectors (e.g., Kozak consensus sequence, miRNA target sites and WPRE).

In one example embodiment, the non-viral vector for use in gene transfer and/or nanoparticle formulations is a lipid. In one example embodiment the non-viral lipid vector may comprise: 1,2-Dioleoyl-sn-glycero-3-phosphatidylcholine; 1,2-Dioleoyl-sn-glycero-3-phosphatidylethanolamine; Cholesterol; N-[1-(2,3-Dioleyloxy) propyl]N,N, N-trimethylammonium chloride; 1,2-Dioleoyloxy-3-trimethylammonium-propane; Dioctadecylamidoglycylspermine; N-(3-Aminopropyl)-N,N-dimethyl-2,3-bis(dodecyloxy)-1-propanaminium bromide; Cetyltrimethylammonium bromide; 6-Lauroxyhexyl ornithinate; 1-(2,3-Diolcoyloxypropyl)-2,4,6-trimethylpyridinium; 2,3-Dioleyloxy-N-[2 (sperminecarboxamido-ethyl]-N,N-dimethyl-1-propanaminium trifluoroacetate; 1,2-Diolcyl-3-trimethylammonium-propane; N-(2-Hydroxyethyl)-N,N-dimethyl-2,3-bis(tetradecyloxy)-1-propanaminium bromide; Dimyristooxypropyl dimethyl hydroxyethyl ammonium bromide; 3β-[N—(N′,N′-Dimethylaminoethane)-carbamoyl]cholesterol; Bis-guanidium-tren-cholesterol; 1,3-Diodeoxy-2-(6-carboxy-spermyl)-propylamide; Dimethyloctadecylammonium bromide; Dioctadecylamidoglicylspermidin; rac-[(2,3-Dioctadecyloxypropyl) (2-hydroxyethyl)]-dimethylammonium chloride; rac-[2 (2,3-Dihexadecyloxypropyl-oxymethyloxy)ethyl]trimethylammonium bromide; Ethyldimyristoylphosphatidylcholine; 1,2-Distearyloxy-N,N-dimethyl-3-aminopropane; 1,2-Dimyristoyl-trimethylammonium propane; O,O′-Dimyristyl-N-lysyl aspartate; 1,2-Distearoyl-sn-glycero-3-ethylphosphocholine; N-Palmitoyl D-erythro-sphingosyl carbamoyl-spermine; N-t-Butyl-N0-tetradecyl-3-tetradecylaminopropionamidine; Octadecenolyoxy [ethyl-2-heptadecenyl-3 hydroxyethyl]imidazolinium chloride; N1-Cholesteryloxycarbonyl-3,7-diazanonane-1,9-diamine; 2-(3-[Bis(3-amino-propyl)-amino]propylamino)-N-ditetradecylcarbamoylme-ethyl-acetamide; 1,2-dilinoleyloxy-3-dimethylaminopropane; 2,2-dilinoleyl-4-dimethylaminoethyl-[1,3]-dioxolane; and dilinoleyl-methyl-4-dimethylaminobutyrate.

In one example embodiment, the non-viral vector for use in gene transfer and/or nanoparticle formulations is a polymer. In one example embodiment the non-viral polymer vector may comprise: Poly(ethylene)glycol; Polyethylenimine; Dithiobis(succinimidylpropionate); Dimethyl-3,3′-dithiobispropionimidate; Poly(ethylene imine)biscarbamate; Poly(L-lysine); Histidine modified PLL; Poly(N-vinylpyrrolidone); Poly(propylenimine); Poly(amidoamine); Poly(amido ethylenimine); Triethylenetetramine; Poly(β-aminoester); Poly(4-hydroxy-L-proline ester); Poly(allylamine); Poly(α-[4-aminobutyl]-L-glycolic acid); Poly(D,L-lactic-co-glycolic acid); Poly(N-ethyl-4-vinylpyridinium bromide); Poly(phosphazene)s; Poly(phosphoester)s; Poly(phosphoramidate)s; Poly(N-2-hydroxypropylmethacrylamide); Poly(2-(dimethylamino)ethyl methacrylate); Poly(2-aminoethyl propylene phosphate); Chitosan; Galactosylated chitosan; N-Dodacylated chitosan; Histone; Collagen; and Dextran-spermine.

### Targeted Adipocyte or Adipocyte-Progenitor Delivery

In one example embodiment, gene therapy vectors are used that have tropism for expression in adipocytes or adipocyte progenitors. In another example embodiment, the transcriptional regulatory region may comprise a promoter and, optionally, an enhancer region. Preferably, the promoter is specific for adipose tissue. The enhancer need not be specific for adipose tissue. Alternatively, the transcriptional regulatory region may comprise an adipose tissue-specific promoter and an adipose tissue-specific enhancer. In one embodiment, the tissue-specific promoter is an adipocyte-specific promoter such as, for example, the adipocyte protein 2 (aP2, also known as fatty acid binding protein 4 (FABP4)), the PPARy promoter, the adiponectin promoter, the phosphoenolpyruvate carboxykinase (PEPCK) promoter, the promoter derived from human aromatase cytochrome p450 (p450arom), or the Foxa-2 promoter (see, e.g., Graves R, et al, Genes Dev. 1991; 5:428-437; Ross S, et al, Proc. Natl. Acad. Sci. USA 1990; 87:9590-9594; Simpson E, et al., U.S. Pat. No. 5,446,143; Mahendroo M, et al., J. Biol. Chem. 1993; 268:19463-19470; Simpson E, et al., Clin. Chem. 1993; 39:317-324; and Sasaki H, et al., Cell 1994; 76:103-115). In a preferred embodiment, the enhancer region is selected from the group consisting of the adipose-specific aP2 enhancer and the adipose-specific UCP1 enhancer. In another example embodiment, an adipose-specific promoter is much less potent than that of a ubiquitous promoter. Thus, a ubiquitous promoter, such as hybrid cytomegalovirus enhancer/chicken β-actin (CBA or CAG) or cytomegalovirus (CMV) is used. In another example embodiment, a ubiquitous promoter is used in combination with any adipose targeting strategy described herein or when the vector is administered locally to adipose tissue. In another example embodiment, systemic delivery utilizes an adipose-specific promoter with a higher dose, while local delivery utilizes a CBA or CMV promoter with a lower dosage.

In one embodiment, the vector contains at least one target sequence of at least one miRNA expressed in non-adipose tissue. In another example embodiment, liver- and heart-specific abundant miRNAs are used to de-target or suppress transgene expression in liver and heart by embedding the miRNA target sequences in the vectors, in particular for AAV8 vectors. In one embodiment, the target sequence of at least one miRNA is located in the 3′ untranslated region (3′UTR) of cellular messenger RNA (mRNA). Exemplary target sequences of the at least one miRNA include, but are not limited to miR1 (miRbase database accession numbers MI0000651 and MI0000437), miR122 or miR122a (MI0000442), miR152 (MI0000462), miR199 (MI0000242), miR215 (MI0000291), miR192 (MI0000234), miR148a (MI0000253), miR194 (MI0000488), miR1 (MI0000651), miRT133 (MI0000450), miR206 (MI0000490), miR208 (MI0000251), miR124 (MI0000443), miR125 (MI0000469), miR216 (MI0000292), and miR130 (MI0000448). In preferred embodiments, the miRNA target sites are selected from miRNA122a and miRNA1. In another example embodiment, 1, 2, 3, or 4 repeat target sites for each miRNA can be used. Sequence references are publicly available and may be obtained from the miRbase (www.mirbase.org/). The term “microRNAs” or “miRNAs”, as used herein, are small (˜22-nt), evolutionarily conserved, regulatory RNAs involved in RNA-mediated gene silencing at the post-transcriptional level (see, e.g., Barrel DP. Cell 2004; 116:281-297). Through base pairing with complementary regions (most often in the 3′ untranslated region (3′UTR) of cellular messenger RNA (mRNA)), miRNAs can act to suppress mRNA translation or, upon high-sequence homology, cause the catalytic degradation of mRNA. Because of the highly differential tissue expression of many miRNAs, cellular miRNAs can be exploited to mediate tissue-specific targeting of gene therapy vectors. By engineering tandem copies of target elements perfectly complementary to tissue-specific miRNAs (miRT) within vectors, transgene expression in undesired tissues can be efficiently inhibited.

### Recombinant COBLL1

In another example embodiment, a method for treating subjects at risk for, or suffering from, T2D comprises administering a COBL11 recombinant polypeptide. In certain embodiments, recombinant COBLL1 protein is delivered intracellularly to a subject in need thereof and is used as a protein therapeutic. Protein therapeutics offer high specificity, and the ability to treat “undruggable” targets, in diseases associated with protein deficiencies or mutations (e.g., COBLL1). As used herein COBLL1 protein includes all variants and protein fragments, described further herein. Previous studies have found that COBLL1 interacts with ROR1 (Plešingerová, et al. Expression of COBLL1 encoding novel ROR1 binding partner is robust predictor of survival in chronic c lymphocytic leukemia. Haematologica. 2018; 103 (2): 313-324). Applicants discovered that COBLL1 plays a role in the remodeling of the actin cytoskeleton, specifically, actin remodeling in differentiating adipocytes. Thus, while not being bound by a particular scientific theory, it is expected that administration of functional COBLL1 protein may restore proper actin remodeling in differentiating adipocytes.

COBLL1 has the following domains: WH2, COBL-like, and Cordon-bleu_ubiquitin_domain. The WHI2 (WASP-Homology 2, or Wiskott-Aldrich homology 2) domain is an ˜18 amino acids actin-binding motif. Single WH2 domains can sequester G-actin. COBL contains three G-actin-binding WH2 domains and act as a dynamizer of actin assembly. COBL has profilin-like filament nucleating and severing activities. The Cordon-bleu_ubiquitin_domain protein domain is highly conserved among vertebrates. The sequence contains three repeated lysine, arginine, and proline-rich regions, the KKRAP motif. It is expressed specifically in the node. This domain has a ubiquitin-like fold. In certain embodiments, full length COBLL1 protein is administered. In one example embodiment, a COBL11 sequence selected from Table A is administered. In certain embodiments, a truncated COBLL1 protein is administered. For example, protein domains that function in the nucleus are not required for the recombinant protein (e.g., AR interacting domains). Further, only the actin binding domains and domains required for actin remodeling are required. Various methods can be used for delivery of COBLL1 to adipose cells. In certain embodiments, COBLL1 is delivered in a composition capable of delivering COBLL1 intracellularly.

TABLE A

| Locus | Sequence |
|---|---|
| NP_001352601 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltkslndlgl relyamdvnr escqisqnld imkekenkgf |
| | fsffqrskkk rdqtasapat plvnkhrptf trsntiskpy isntlpsdap kkrraplppm |
| | pasqsvpqdl ahiqerpasc ivksmsvdet dkspceagrv ragslqlssm sagnsslrrt |
| | krkapsppsk ipphqsdens rvtalqpvdg vppdsasean speelsspag issdysleei |
| | dekeelsevp kveaenispk sqdipfvstd iintlkndpd salgngsgef sqnsmeekqe |
| | tkstdgqeph svvydtsngk kvvdsirnlk slgpnqenvv qneiivypen |
| | tednmkngvk kteinvegva knnnidmeve rpsnseahet dtaisykenh |
| | laassvpdqk lnqpsaektk daaiqttpsc nsfdgkhqdh nlsdskveec vqtsnnnist |
| | qhsclssqds vntsrefrsq gtliihsedp ltvkdpicah gnddllppvd ridknstasy |
| | lknyplyrqd ynpkpkpsne itreyipkig mttykivppk sleiskdwqs etieykddqd |
| | mhalgkkhth envketaiqt edsaisespe eplpnlkpkp nlrtehqvps svsspddamv |
| | splkpapkmt rdtgtapfap nleeinnile skfksrasna qakpssfflq mqkrvsghyv |
| | tsaaaksvha apnpapkelt nkeaerdmlp speqtlspls kmphsvpqpl vektdddvig |
| | qapaeasppp iapkpvtipa sqvstqnlkt lktfgaprpy sssgpspfal avvkrsqsfs |
| | kertespsas alvqppante egkthsvnkf vdipqlgvsd kennsahneq nsqiptptdg |
| | psftvmrqss ltfqssdpeq mrqslltair sgeaaaklkr vtipsntisv ngrsrlshsm |
| | spdaqdgh (SEQ ID NO: 1) |
| NP_055715 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltkslndlgl relyamdvnr escqisqnld imkekenkgf |
| | fsffqrskkk rdqtasapat plvnkhrptf trsntiskpy isntlpsdap kkrraplppm |
| | pasqsvpqdl ahiqerpasc ivksmsvdet dkspceagrv ragslqlssm sagnsslrrt |
| | krkapsppsk ipphqsdens rvtalqpvdg vppdsasean speelsspet fhpglssqeq |
| | ctapklmeet svfecpgtpe aaitsltsgi ssdysleeid ekeelsevpk veaenispks |
| | qdipfvstdi intlkndpds algngsgefs qnsmeekqet kstdgqephs vvydtsngkk |
| | vvdsirnlks lgpnqenvqn eiivypente dnmkngvkkt einvegvakn nnidmeverp |
| | snseahetdt aisykenhla assvpdqkln qpsaektkda aiqttpscns fdgkhqdhnl |
| | sdskveecvq tsnnnistqh sclssqdsvn tsrefrsqgt liihsedplt vkdpicahgn |
| | ddllppvdri dknstasylk nyplyrqdyn pkpkpsneit reyipkigmt tykivppksl |
| | eiskdwqset ieykddqdmh algkkhthen vketaiqted saisespeep lpnlkpkpnl |
| | rtehqvpssv sspddamvsp lkpapkmtrd tgtapfapnl eeinnilesk fksrasnaqa |
| | kpssfflqmq krvsghyvts aaaksvhaap npapkeltnk eaerdmlpsp eqtlsplskm |
| | phsvpqplve ktdddvigqa paeaspppia pkpvtipasq vstqnlktlk tfgaprpyss |
| | sgpspfalav vkrsqsfske rtespsasal vqppanteeg kthsvnkfvd ipqlgvsdke |
| | nnsahneqns qiptptdgps ftvmrqsslt fqssdpeqmr qslltairsg eaaaklkrvt |
| | ipsntisvng rsrlshsmsp daqdgh (SEQ ID NO: 2) |
| NP_001265387 | mppswsplmc graaeaaass rtpgremgqa vtrrlgagar aaprramdgr tprpqdapar |
| | reiagswrkp kakaplppae tkytdvssaa dsvestafim eqkenmidkd velsvvlpgd |
| | iiksttvhgs kpmmdllifl caqyhlnpss ytidllsaeq nhikfkpntp igmlevekvi |
| | lkpkmldkkk ptpiipektv rvvinfkktq ktivrvspha slqelapiic skcefdplht |
| | lllkdyqsqe pldltksind lglrelyamd vnratsvtvf sksslqescq isqnldimke |
| | kenkgffsff qrskkkrdqt asapatplvn khrptftrsn tiskpyisnt lpsdapkkrr |
| | aplppmpasq svpqdlahiq erpascivks msvdetdksp ceagrvrags lqlssmsagn |
| | sslrrtkrka psppskipph qsdensrvta lqpvdgvppd saseanspee lsspetfhpg |
| | lssqeqctap klmeetsvfe cpgtpeaait sltsgissdy sleeidekee lsevpkveae |
| | nispksqdip fvstdiintl kndpdsalgn gsgefsqnsm eekqetkstd gqephsvvyd |
| | tsngkkvvds irnlkslgpn qenvvqneii vypentednm kngvkktein vegvaknnni |
| | dmeverpsns eahetdtais ykenhlaass vpdqklnqps aektkdaaiq ttpscnsfdg |
| | khqdhnlsds kveecvqtsn nnistqhscl ssqdsvntsr efrsqgtlii hsedpltvkd |
| | picahgnddl lppvdridkn stasylknyp lyrqdynpkp kpsneitrey ipkigmttyk |
| | ivppksleis kdwqsetiey kddqdmhalg kkhthenvke taiqtedsai sespeeplpn |
| | lkpkpnlrte hqvpssvssp ddamvsplkp apkmtrdtgt apfapnleei nnileskfks |
| | rasnaqakps sfflqmqkrv sghyvtsaaa ksvhaapnpa pkeltnkeae rdmlpspeqt |
| | lsplskmphs vpqplvektd ddvigqapae aspppiapkp vtipasqvst qnlktlktfg |
| | aprpysssgp spfalavvkr sqsfskerte spsasalvqp panteegkth svnkfvdipq |
| | lgvsdkenns ahneqnsqip tptdgpsftv mrqssltfqs sdpeqmrqsl ltairsgeaa |
| | aklkrvtips ntisvngrsr lshsmspdaq dgh (SEQ ID NO: 3) |
| NP_001265389 | mppswsplmc graaeaaass rtpgremgqa vtrrlgagar aaprramdgr tprpqdapar |
| | rkpkakaplp paetkytdvs saadsvesta fimeqkenmi dkdvelsvvl pgdiiksttv |
| | hgskpmmdll iflcaqyhln pssytidlls aeqnhikfkp ntpigmleve kvilkpkmld |
| | kkkptpiipe ktvrvvinfk ktqktivrvs phaslqelap iicskcefdp lhtlllkdyq |
| | sqepldltks lndlglrely amdvnrescq isqnldimke kenkgffsff qrskkkrdqt |
| | asapatplvn khrptftrsn tiskpyisnt lpsdapkkrr aplppmpasq svpqdlahiq |
| | erpascivks msvdetdksp ceagrvrags lqlssmsagn sslrrtkrka psppskipph |
| | qsdensrvta lqpvdgvppd saseanspee lsspagissd ysleeideke elsevpkvea |
| | enispksqdi pfvstdiint lkndpdsalg ngsgefsqns meekqetkst dgqephsvvy |
| | dtsngkkvvd sirnlkslgp nqenvvqnei ivypentedn mkngvkktei nvegvaknnn |
| | idmeverpsn seahetdtai sykenhlaas svpdqklnqp saektkdaai qttpscnsfd |
| | gkhqdhnlsd skveecvqts nnnistqhsc lssqdsvnts refrsqgtli ihsedpltvk |
| | dpicahgndd llppvdridk nstasylkny plyrqdynpk pkpsneitre yipkigmtty |
| | kivppkslei skdwqsetie ykddqdmhal gkkhthenvk etaiqtedsa isespeeplp |
| | nlkpkpnlrt ehqvpssvss pddamvsplk papkmtrdtg tapfapnlee innileskfk |
| | srasnaqakp ssfflqmqkr vsghyvtsaa aksvhaapnp apkeltnkea erdmlpspeq |
| | tlsplskmph svpqplvekt dddvigqapa caspppiapk pvtipasqvs tqnlktlktf |
| | gaprpysssg pspfalavvk rsqsfskert espsasalvq ppanteegkt hsvnkfvdip |
| | qlgvsdkenn sahneqnsqi ptptdgpsft vmrqssltfq ssdpeqmrqs lltairsgea |
| | aaklkrvtip sntisvngrs rlshsmspda qdgh (SEQ ID NO: 4) |
| NP_001265390 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltkslndlgl relyamdvnr escqisqnld imkekenkgf |
| | fsffqrskkk rdqtasapat plvnkhrptf trsntiskpy isntlpsdap kkrraplppm |
| | pasqsvpqdl ahiqerpasc ivksmsvdet dkspceagrv ragslqlssm sagnsslrrt |
| | krkapsppsk ipphqsdens rvtalqpvdg vppdsasean speelsspag issdysleei |
| | dekeelsevp kveaenispk sqdipfvstd iintlkndpd salgngsgef sqnsmeekqe |
| | tkstdgqeph svvydtsngk kvvdsirnlk slgpnqenvv qneiivypen tednmkngvk |
| | kteinvegva knnnidmeve rpsnseahet dtaisykenh laassvpdqk lnqpsaektk |
| | daaiqttpsc nsfdgkhqdh nlsdskveec vqtsnnnist qhsclssqds vntsrefrsq |
| | gtliihsedp ltvkdpicah gnddllppvd ridknstasy lknyplyrqd ynpkpkpsne |
| | itreyipkig mttykivppk sleiskdwqs etieykddqd mhalgkkhth envketaiqt |
| | edsaisespe eplpnlkpkp nlrtehqvps svsspddamv splkpapkmt rdtgtapfap |
| | nleeinnile skfksrasna qakpssfflq mqkrvsghyv tsaaaksvha apnpapkelt |
| | nkeaerdmlp speqtlspls kmphsvpqpl vektdddvig qapacasppp iapkpvtipa |
| | sqvstqnlkt lktfgaprpy sssgpspfal avvkrsqsfs kertespsas alvqppante |
| | egkthsvnkf vdipqlgvsd kennsahneq nsqiptptdg psftvmrqss ltfqssdpeq |
| | mrqslltair sgeaaaklkr vtipsntisv ngrsrlshsm spdaqdgh |
| | (SEQ ID NO: 5) |
| NP_001352599 | mppswsplmc graaeaaass rtpgremgqa vtrrlgagar aaprramdgr tprpqdapar |
| | rkpkakaplp paetkytdvs saadsvesta fimeqkenmi dkdvelsvvl pgdiiksttv |
| | hgskpmmdll iflcaqyhln pssytidlls aeqnhikfkp ntpigmleve kvilkpkmld |
| | kkkptpiipe ktvrvvinfk ktqktivrvs phaslqelap iicskcefdp lhtlllkdyq |
| | sqepldltks lndlglrely amdvnrescq isqnldimke kenkgffsff qrskkkrdqt |
| | asapatplvn khrptftrsn tiskpyisnt lpsdapkkrr aplppmpasq svpqdlahiq |
| | erpascivks msvdetdksp ceagrvrags lqlssmsagn sslrrtkrka psppskipph |
| | qsdensrvta lqpvdgvppd saseanspee lsspagissd ysleeideke elsevpkvea |
| | enispksqdi pfvstdiint lkndpdsalg ngsgefsqns meekqetkst dgqephsvvy |
| | dtsngkkvvd sirnlkslgp nqenvqneii vypentednm kngvkktein vegvaknnni |
| | dmeverpsns eahetdtais ykenhlaass vpdqklnqps aektkdaaiq ttpscnsfdg |
| | khqdhnlsds kveecvqtsn nnistqhscl ssqdsvntsr efrsqgtlii hsedpltvkd |
| | picahgnddl lppvdridkn stasylknyp lyrqdynpkp kpsneitrey ipkigmttyk |
| | ivppksleis kdwqsetiey kddqdmhalg kkhthenvke taiqtedsai sespeeplpn |
| | lkpkpnlrte hqvpssvssp ddamvsplkp apkmtrdtgt apfapnleei nnileskfks |
| | rasnaqakps sfflqmqkrv sghyvtsaaa ksvhaapnpa pkeltnkeae rdmlpspeqt |
| | lsplskmphs vpqplvektd ddvigqapae aspppiapkp vtipasqvst qnlktlktfg |
| | aprpysssgp spfalavvkr sqsfskerte spsasalvqp panteegkth svnkfvdipq |
| | lgvsdkenns ahneqnsqip tptdgpsftv mrqssltfqs sdpeqmrqsl ltairsgeaa |
| | aklkrvtips ntisvngrsr lshsmspdaq dgh (SEQ ID NO: 6) |
| NP_001352600 | mppswsplmc graaeaaass rtpgremgqa vtrrlgagar aaprramdgr tprpqdapar |
| | rkpkakaplp paetkytdvs saadsvesta fimeqkenmi dkdvelsvvl pgdiiksttv |
| | hgskpmmdll iflcaqyhln pssytidlls aeqnhikfkp ntpigmleve kvilkpkmld |
| | kkkptpiipe ktvrvvinfk ktqktivrvs phaslqelap iicskcefdp lhtlllkdyq |
| | sqepldltks lndlglrely amdvnratsv tvfsksslqe scqisqnldi mkekenkgff |
| | sffqrskkkr dqtasapatp lvnkhrptft rsntiskpyi sntlpsdapk krraplppmp |
| | asqsvpqdla hiqerpasci vksmsvdetd kspceagrvr agslqlssms agnsslrrtk |
| | rkapsppski pphqsdensr vtalqpvdgv ppdsaseans peelsspagi ssdysleeid |
| | ekeelsevpk veaenispks qdipfvstdi intlkndpds algngsgefs qnsmeekqet |
| | kstdgqephs vvydtsngkk vvdsirnlks lgpnqenvvq neiivypent ednmkngvkk |
| | teinvegvak nnnidmever psnseahetd taisykenhl aassvpdqkl nqpsaektkd |
| | aaiqttpscn sfdgkhqdhn lsdskveecv qtsnnnistq hsclssqdsv ntsrefrsqg |
| | tliihsedpl tvkdpicahg nddllppvdr idknstasyl knyplyrqdy npkpkpsnei |
| | treyipkigm ttykivppks leiskdwqse tieykddqdm halgkkhthe nvketaiqte |
| | dsaisespee plpnlkpkpn lrtehqvpss vsspddamvs plkpapkmtr dtgtapfapn |
| | leeinniles kfksrasnaq akpssfflqm qkrvsghyvt saaaksvhaa pnpapkeltn |
| | keaerdmlps peqtlsplsk mphsvpqplv ektdddvigq apaeaspppi apkpvtipas |
| | qvstqnlktl ktfgaprpys ssgpspfala vvkrsqsfsk ertespsasa lvqppantee |
| | gkthsvnkfv dipqlgvsdk ennsahneqn sqiptptdgp sftvmrqssl tfqssdpeqm |
| | rqslltairs geaaaklkrd g (SEQ ID NO: 7) |
| NP_001352602 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltkslndlgl relyamdvnr escqisqnld imkekenkgf |
| | fsffqrskkk rdqtasapat plvnkhrptf trsntiskpy isntlpsdap kkrraplppm |
| | pasqsvpqdl ahiqerpasc ivksmsvdet dkspceagrv ragslqlssm sagnsslrrt |
| | krkapsppsk ipphqsdens rvtalqpvdg vppdsasean speelsspag issdysleei |
| | dekeelsevp kveaenispk sqdipfvstd iintlkndpd salgngsgef sqnsmeekqe |
| | tkstdgqeph svvydtsngk kvvdsimnlk slgpnqenvv qneiivypen tednmkngvk |
| | kteinvegva knnnidmeve rpsnseahet dtaisykenh laassvpdqk lnqpsaektk |
| | daaiqttpsc nsfdgkhqdh nlsdskveec vqtsnnnist qhsclssqds vntsrefrsq |
| | gtliihsedp ltvkdpicah gnddllppvd ridknstasy lknyplyrqd ynpkpkpsne |
| | itreyipkig mttykivppk sleiskdwqs etieykddqd mhalgkkhth envketaiqt |
| | edsaisespe eplpnlkpkp nlrtehqvps svsspddamv splkpapkmt rdtgtapfap |
| | nleeinnile skfksrasna qakpssfflq mqkrvsghyv tsaaaksvha apnpapkelt |
| | nkeaerdmlp speqtlspls kmphsvpqpl vektdddvig qapaeasppp iapkpvtipa |
| | sqvstqnlkt lktfgaprpy sssgpspfal avvkrsqsfs kertespsas alvqppante |
| | egkthsvnkf vdipqlgvsd kennsahneq nsqiptptdg psftvmrqss ltfqssdpeq |
| | mrqslltair sgeaaaklkr vtipsntisv ngrsrlshsm spdaqdgh |
| | (SEQ ID NO: 8) |
| NP_001352603 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltksindlgl relyamdvnr atsvtvfsks slqescqisq |
| | nldimkeken kgffsffqrs kkkrdqtasa patplvnkhr ptftrsntis kpyisntlps |
| | dapkkrrapl ppmpasqsvp qdlahiqerp ascivksmsv detdkspcea grvragslql |
| | ssmsagnssl rrtkrkapsp pskipphqsd ensrvtalqp vdgvppdsas eanspeelss |
| | pagissdysl eeidekeels evpkveaeni spksqdipfv stdiintlkn dpdsalgngs |
| | gefsqnsmee kqetkstdgq ephsvvydts ngkkvvdsir nlkslgpnqe nvvqneiivy |
| | pentednmkn gvkkteinve gvaknnnidm everpsnsea hetdtaisyk enhlaassvp |
| | dqklnqpsae ktkdaaiqtt pscnsfdgkh qdhnlsdskv eecvqtsnnn istqhsclss |
| | qdsvntsref rsqgtliihs edpltvkdpi cahgnddllp pvdridknst asylknyply |
| | rqdynpkpkp sneitreyip kigmttykiv ppksleiskd wqsetieykd dqdmhalgkk |
| | hthenvketa iqtedsaise speeplpnlk pkpnlrtehq vpssvsspdd amvsplkpap |
| | kmtrdtgtap fapnleeinn ileskfksra snaqakpssf flqmqkrvsg hyvtsaaaks |
| | vhaapnpapk eltnkeaerd mlpspeqtls plskmphsvp qplvektddd vigqapaeas |
| | pppiapkpvt ipasqvstqn lktlktfgap rpysssgpsp falavvkrsq sfskertesp |
| | sasalvqppa nteegkthsv nkfvdipqlg vsdkennsah neqnsqiptp tdgpsftvmr |
| | qssltfqssd peqmrqsllt airsgeaaak lkrvtipsnt isvngrsrls hsmspdaqdg |
| | h (SEQ ID NO: 9) |
| NP_001352604 | mdgrtprpqd aparrkpkak aplppaetky tdvssaadsv estafimeqk enmidkdvel |
| | svvlpgdiik sttvhgskpm mdlliflcaq yhlnpssyti dllsaeqnhi kfkpntpigm |
| | levekvilkp kmldkkkptp iipektvrvv infkktqkti vrvsphaslq elapiicskc |
| | efdplhtlll kdyqsqepld ltksindlgl relyamdvnr atsvtvfsks slqescqisq |
| | nldimkeken kgffsffqrs kkkrdqtasa patplvnkhr ptftrsntis kpyisntlps |
| | dapkkrrapl ppmpasqsvp qdlahiqerp ascivksmsv detdkspcea grvragslql |
| | ssmsagnssl rrtkrkapsp pskipphqsd ensrvtalqp vdgvppdsas eanspeelss |
| | pagissdysl eeidekeels evpkveaeni spksqdipfv stdiintlkn dpdsalgngs |
| | gefsqnsmee kqetkstdgq ephsvvydts ngkkvvdsir nlkslgpnqe nvvqneiivy |
| | pentednmkn gvkkteinve gvaknnnidm everpsnsea hetdtaisyk enhlaassvp |
| | dqklnqpsae ktkdaaiqtt pscnsfdgkh qdhnlsdskv eecvqtsnnn istqhsclss |
| | qdsvntsref rsqgtliihs edpltvkdpi cahgnddllp pvdridknst asylknyply |
| | rqdynpkpkp sneitreyip kigmttykiv ppksleiskd wqsetieykd dqdmhalgkk |
| | hthenvketa iqtedsaise speeplpnlk pkpnlrtehq vpssvsspdd amvsplkpap |
| | kmtrdtgtap fapnleeinn ileskfksra snaqakpssf flqmqkrvsg hyvtsaaaks |
| | vhaapnpapk eltnkeaerd mlpspeqtls plskmphsvp qplvektddd vigqapaeas |
| | pppiapkpvt ipasqvstqn lktlktfgap rpysssgpsp falavvkrsq sfskertesp |
| | sasalvqppa nteegkthsv nkfvdipqlg vsdkennsah neqnsqiptp tdgpsftvmr |
| | qssltfqssd peqmrqsllt airsgeaaak lkrvtipsnt isvngrsrls hsmspdaqdg |
| | (SEQ ID NO: 10) |

### Recombinant BCL2

In another example embodiment, a method for treating subjects at risk for, or suffering from, lipodystrophy comprises administering a BCL2 recombinant polypeptide. In certain embodiments, recombinant BCL2 protein is delivered intracellularly to a subject in need thereof and is used as a protein therapeutic. Protein therapeutics offer high specificity, and the ability to treat “undruggable” targets, in diseases associated with protein deficiencies or mutations (e.g., BCL2). As used herein BCL2 protein includes all variants and protein fragments, described further herein. Previous studies have found that BCL2 promotes and inhibits apoptosis, and that the BCL-2 family proteins are evolutionary conserved and share BCL2 homology (BH) domains. Choudhury, A comparative analysis of BCL-2 family, Bioinformation. 2019; 15 (4): 299-306. In an aspect, the BCL2 is selected from three groups based on their primary function (1) anti-apoptotic proteins (BCL-2, BCL-XL, BCL-W, MCL-1, BFL-1/A1), (2) pro-apoptotic pore-formers (BAX, BAK, BOK) and (3) pro-apoptotic BH3-only proteins (BAD, BID, BIK, BIM, BMF, HRK, NOXA, PUMA, etc.). In an aspect, the BCL-2 comprises a BH3 domain. In embodiments, the BCL-2 protein is an anti-apoptotic or pore-former protein and comprises BH1, BH2, BH3 and BH4 domain. Sec, e.g., Kale, J., Osterlund, E. & Andrews, D. BCL-2 family proteins: changing partners in the dance towards death. Cell Death Differ 25, 65 80 (2018). Residues of the domains in BCL-2 are generally conserved: BIII (residues 136-155), BH2 (187-202), BH3 (93-107) and BH4 (10-30). See, e.g., Reed J C, Zha H, Aime-Sempe C, Takayama S, Wang H G. Structure-function analysis of Bcl-2 family proteins. Regulators of programmed cell death. Adv Exp Med Biol. 1996; 406:99-112. In an aspect, the BCL-2 is an anti-apoptotic protein and comprises both BH1 and BH2 domains. In an aspect, the BCL-2 protein may be truncated at the BH4 domain.

As disclosed herein, the variant causes BCL2 to be reduced in Subcutaneous AMSCs and skeletal muscle. The reduction is in the stem cells at day 0, but the effect on increased apoptosis is seen in mature adipocytes. Thus, while not being bound by a particular scientific theory, it is expected that administration of functional BCL2 protein may improve or enhance modulation of disease susceptibility in T2D. In an aspect, the administration of BCL-2 is provided when the risk allele rs12454712 is present.

In certain embodiments, full length BCL2 protein is administered. In one example embodiment, a BCL2 sequence selected from Table 2 is administered. In certain embodiments, a truncated BCL2 protein is administered. In an aspect, an isoform of a BCL-2 or BCL-2-like protein, for example, BCL2L1, BCL2L2, BCL2L10, BCL2L12, BCL2L13, BCL2L14, BCL2L15 is provided. Various methods can be used for delivery of BCL2 to adipose cells. In certain embodiments, BCL2 is delivered in a composition capable of delivering BCL2 intracellularly. In embodiments, BCL2 is administered to skeletal muscle or AMSCs.

TABLE B

| Locus | Sequence |
|---|---|
| NM_0006 | MAHAGRTGYD NREIVMKYIH YKLSQRGYEW |
| 33.3 | DAGDVGAAPP GAAPAPGIFS SQPGHTPHPA |
| | ASRDPVARTS PLQTPAAPGA AAGPALSPVP |
| | PVVHLTLRQA GDDFSRRYRR DFAEMSSQLH |
| | LTPFTARGRF ATVVEELFRD GVNWGRIVAF |
| | FEFGGVMCVE SVNREMSPLV DNIALWMTEY |
| | LNRHLHTWIQ DNGGWDAFVE LYGPSMRPLF |
| | DFSWLSLKTL LSLALVGACI TLGAYLGHK |
| | (SEQ ID NO: 11) |
| NP_00064 | MAHAGRTGYDNREIVMKYIHYKLSQRGYEWDA |
| 8.2 | GDVGAAPPGAAPAPGIFSSQPGHTPHPAASRD |
| | PVARTSPLQTPAAPGAAAGPALSPVPPVVHLT |
| | LRQAGDDFSRRYRRDFAEMSSQLHLTPFTARG |
| | RFATVVEELFRDGVNWGRIVAFFEFGGVMCVE |
| | SVNREMSPLVDNIALWMTEYLNRHLHTWIQDN |
| | GGWVGALGDVSLG (SEQ ID NO: 12) |
| Q07817- | msqsnrelvv dflsyklsqk gyswsqfsdv |
| 01 | eenrteapeg tesemetpsa ingnpswhla |
| (NP_0013 | dspavngatg hsssldarev ipmaavkqal |
| 04848.1) | reagdefelr yrrafsdlts qlhitpgtay |
| | qsfeqvvnel frdgvnwgri vaffsfggal |
| | cvesvdkemq vlvsriaawm atylndhlep |
| | wiqenggwdt fvelygnnaa aesrkgqerf |
| | nrwfltgmtv agvvllgslf srk |
| | (SEQ ID NO: 13) |
| NP_00118 | msqsnrelvv dflsyklsqk gyswsqfsdv |
| 2.1 | eenrteapeg tesemetpsa ingnpswhla |
| | dspavngatg hsssldarev ipmaavkqal |
| | reagdefelr yrrafsdlts qlhitpgtay |
| | qsfeqdtfve lygnnaaaes rkgqerfnrw |
| | fltgmtvagv vllgslfsrk |
| | (SEQ ID NO: 14) |
| Q07817-3 | MSQSNRELVV DFLSYKLSQK GYSWSQFSDV |
| | EENRTEAPEG TESEMETPSA INGNPSWHLA |
| | DSPAVNGATG HSSSLDAREV IPMAAVKQAL |
| | REAGDEFELR YRRAFSDLTS QLHITPGTAY |
| | QSFEQVVNEL FRDGVNWGRI VAFFSFGGAL |
| | CVESVDKEMQ VLVSRIAAWM ATYLNDHLEP |
| | WIQENGGWVR TKPLVCPFSL ASGQRSPTAL |
| | LLYLFLLCWV IVGDVDS |
| | (SEQ ID NO: 15) |
| Q07817-2 | MSQSNRELVV DFLSYKLSQK GYSWSQFSDV |
| | EENRTEAPEG TESEMETPSA INGNPSWHLA |
| | DSPAVNGATG HSSSLDAREV IPMAAVKQAL |
| | REAGDEFELR YRRAFSDLTS QLHITPGTAY |
| | QSFEQDTFVE LYGNNAAAES RKGQERFNRW |
| | FLTGMTVAGV VLLGSLFSRK |
| | (SEQ ID NO: 16) |
| NP_62004 | mcstsgcdle eiplddddln tiefkilayy |
| 8.1 | trhhvfkstp alfspkllrt rslsqrglgn |
| | csaneswtev swpcrnsqss ekainlgkkk |
| | sswkaffgvv ekedsqstpa kvsaqgqrtl |
| | eyqdshsqqw srclsnveqc leheavdpkv |
| | isianrvaei vyswpppqat qaggfkskei |
| | fvteglsfql qghvpvasss kkdeeeqila |
| | kivellkysg dqlerklkkd kalmghfqdg |
| | lsysvfktit dqvlmgvdpr gesevkaqgf |
| | kaalvidvta kltaidnhpm nrvlgfgtky |
| | lkenfspwiq qhggwekilg isheevd |
| | (SEQ ID NO: 17) |
| NP_06512 | mvdqlrertt madplrerte llladylgyc |
| 9 | arepgtpepa pstpeaavlr saaarlrqih |
| | rsffsaylgy pgnrfelval madsvlsdsp |
| | gptwgrvvtl vtfagtller gplvtarwkk |
| | wgfqprlkeq egdvardcqr lvallssrlm |
| | gqhrawlqaq ggwdgfchff rtpfplafwr |
| | kglvqaflsc llttafiylw trll |
| | (SEQ ID NO: 18) |
| NP_00126 | mgrpaglfpp lcpflgfrpe acwerhmqie |
| 9445.1 | rapsvppflr wagyrpgpvr rrgkvelikf |
| | vrvqwrrpqv ewrrrrwgpg pgasmagsee |
| | lglredtlrv laaflrrgea agspvptppr |
| | spaqeeptdf lsrlrrclpc slgrgaapse |
| | sprpcslpir pcyglepgpa tpdfyalvaq |
| | rleqlvqeql ksppspelqg ppstekeail |
| | rrlvalleee aevinqkegi lavspvdlnl |
| | pld (SEQ ID NO: 19) |

### Recombinant KDSR

In an example embodiment, a method for treating subjects at risk for, or suffering from, lipodystrophy comprises administering a 3-ketodihydrosphingosine reductase (KDSR) recombinant polypeptide. In certain embodiments, recombinant KDSR protein is delivered intracellularly to a subject in need thereof and is used as a protein therapeutic. Protein therapeutics offer high specificity, and the ability to treat “undruggable” targets, in diseases associated with protein deficiencies or mutations (e.g., KDSR). As used herein KDSR protein includes all variants and protein fragments, described further herein. In an aspect, KDSR comprises the sequence

(SEQ ID NO: 20)
MLLLAAAFLVAFVLLLYMVSPLISPKPLALPGAHVVVTGGSSGIGKCIAI
ECYKQGAFITLVARNEDKLLQAKKEIEMHSINDKQVVLCISVDVSQDYNQ
VENVIKQAQEKLGPVDMLVNCAGMAVSGKFEDLEVSTFERLMSINYLGSV
YPSRAVITTMKERRVGRIVFVSSQAGQLGLFGFTAYSASKFAIRGLAEAL
QMEVKPYNVYITVAYPPDTDTPGFAEENRTKPLETRLISETTSVCKPEQV
AKQIVKDAIQGNFNSSLGSDGYMLSALTCGMAPVTSITEGLQQVVTMGLF
RTIALFYLGSFDSIVRRCMMQREKSENADKTA.

Previous studies have found that KDSR putative active site residues of the encoded protein are found on the cytosolic side of the endoplasmic reticulum membrane. Key structural elements of KDSR include transmembrane anchors near the N-terminal and C-terminal ends of the protein, Rossman folds, and a highly conserved domain containing three putative catalytic sites. See, e.g., Bariana, T. K., et al. (2019). Sphingolipid dysregulation due to lack of functional KDSR impairs proplatelet formation causing thrombocytopenia. Haematologica, 104 (5), 1036-1045. Doi: 10.3324/haematol.2018.20478. The TyrXXXLys, Asn, and Ser residues form the canonical catalytic triad, and the putative NAD binding site is identified as ThrGlyXXXGlyxGly (SEQ ID NO: 21). See, Boyden et al., Mutations in KDSR Cause Recessive Progressive Symmetric Erythrokeratoderma, The American Journal of Human Genetics 100, 978 984, Jun. 1, 2017; doi: 0.1016/j.ajhg.2017.05.003. Applicants discovered that KDSR plays a role in adipocytes. Thus, while not being bound by a particular scientific theory, it is expected that administration of functional KDSR protein may provide treatment for metabolic disease, alone or in combination with BCL2, and/or COBLL1.

In certain embodiments, full length KDSR protein is administered. In certain embodiments, a truncated KDSR protein is administered. Various methods can be used for delivery of KDSR to adipose cells. In certain embodiments, KDSR is delivered in a composition capable of delivering KDSR intracellularly, in an aspect delivered to AMSCs.

### Recombinant VPS4B

In an example embodiment, a method for treating subjects at risk for, or suffering from, lipodystrophy comprises reducing the expression or activity of a Vacuolar protein sorting-associated protein 4B (VPS4B) recombinant polypeptide. As used herein, VPS4B protein includes all variants and protein fragments, described further herein. In an aspect, VPS4B the comprises sequence:

(SEQ ID NO: 22)
MSSTSPNLQKAIDLASKAAQEDKAGNYEEALQLYQHAVQYFLHVVKYEAQ
GDKAKQSIRAKCTEYLDRAEKLKEYLKNKEKKAQKPVKEGQPSPADEKGN
DSDGEGESDDPEKKKLQNQLQGAIVIERPNVKWSDVAGLEGAKEALKEAV
ILPIKFPHLFTGKRTPWRGILLFGPPGTGKSYLAKAVATEANNSTFFSIS
SSDLVSKWLGESEKLVKNLFQLARENKPSIIFIDEIDSLCGSRSENESEA
ARRIKTEFLVQMQGVGVDNDGILVLGATNIPWVLDSAIRRRFEKRIYIPL
PEPHARAAMFKLHLGTTQNSLTEADFRELGRKTDGYSGADISIIVRDALM
QPVRKVQSATHFKKVRGPSRADPNHLVDDLLTPCSPGDPGAIEMTWMDVP
GDKLLEPVVSMSDMLRSLSNTKPTVNEHDLLKLKKFTEDFGQEG.

Vps4 is an adenosine triphosphatase associated with diverse cellular activities (AAA) family member, a subfamily of the AAA+ superfamily. AAA+ ATPases function in assembly/disassembly of protein complexes, protein transport and protein degradation. See, e.g. Ogura T, Wilkinson A J. AAA+ superfamily ATPases: common structure-diverse function. Genes Cells 2001; 6:575-597. The VSP4B is a mammalian homologue of Vps4p, and is also referred to suppressor of K I transport growth defect (SKD1). The VPS4B comprises an AAA domain which is further divided into an alpha/beta domain and an alpha helical domain, a beta-domain inserted with the AAA alpha helical domain and a C-terminal alpha helix (helix alpha10). Sec, Inoue et al., Traffic (2008) 9:12, 2180-2189. The apo form of human VPS4B, which shows 96% amino acid sequence identity with mouse SKD1; however, the human VPS4B structure comprises an N-terminal beta strand structure, an N-terminal region (residues 1 122) including the microtubule-interacting and trafficking (MIT) domain, and comprise a/β domains (residues 123-300 and 425-444).

Applicants discovered that increased expression or activity of VPS4B plays a role in lipid-accumulating cells, for example increased expression is associated with risk or presence of metabolic disease. Thus, while not being bound by a particular scientific theory, it is expected that administration of a catalytically inactive VPS4B or a molecule that inhibits VPS4B may be used for treatment of subjects suffering or at risk from metabolic disease. In an aspect, the VPS4B comprises one or more mutations, In one embodiment, inhibition of VPS4B function is by short hairpin VPS4B (sh-VPS4B) or expression of dominant negative VPS4B (E235Q) See, Lin et al., Identification of an AAA ATPase VPS4B-Dependent Pathway That Modulates Epidermal Growth Factor Receptor Abundance and Signaling during Hypoxia, (2012) Mol. And Cell. Biol. 32:6 1124-1138; doi: 10.1128/MCB.06053-11. In certain embodiments, a short hairpin VPS4B protein is administered.

### Gene Editing of Risk Variants

In one embodiment, a method of treating subjects at risk for, or suffering from, T2D comprises administering a gene editing system that corrects one or more genomic variants that decrease the expression of COBL11 in adipocyte and/or adipocyte progenitors. In one example embodiment, the gene editing system is used to edit one or more variants that reduce COBL11 expression. In one example embodiment, the one or more variants reduce binding of POU2FA to an enhancer controlling COBL11 expression. In another example embodiment, the gene editing system is used to edit a rs6712203 variant from C to T. In one embodiment, a method of treating subjects at risk for, or suffering from, lipodystrophy comprises administering a gene editing system that corrects one or more genomic variants that decrease the expression of BCL2 in adipose-derived mesenchymal stem cells (AMSCs) or skeletal muscle and/or KDSR in ASMCs. In one example embodiment, the gene editing system is used to edit one or more variants that reduce BCL2 and/or KDSR expression. In one embodiment, a method of treating subjects at risk for, or suffering from, lipodystrophy comprises administering a gene editing system that corrects one or more genomic variants that increase the expression of VPS4B in ASMCs. In one example embodiment, the gene editing system is used to edit one or more variants that increase VPS4B. In another example embodiment, the gene editing system is used to edit a rs12454712 variant from T to C.

### Programmable Nucleases

In certain example embodiments, a programmable nuclease may be used to edit a genomic region comprising one or more genomic variants associated with decreased expression or activity of COBLL1 in adipocyte or adipocyte progenitors. In certain example embodiments, a programmable nuclease may be used to edit a genomic region comprising one or more genomic variants associated with increased expression or activity of VPS4B in ASMCs. In example embodiments, a programmable nuclease may be used to edit a genomic region comprising one or more genomic variants associated with decreased expression or activity of BCL2 in skeletal muscle, or with decreased expression or activity of BCL2 or KDSR in ASMCs. Gene editing using programmable nucleases may utilize two different cell repair pathways, non-homologous end joining (NHEJ) and homology directed repair. In certain example embodiment, HDR is used to provide template that replaces a genomic region comprising the variant with a donor that edits the risk variant to a wild-type or non-risk variant. Example programmable nucleases for use in this manner include zinc finger nucleases (ZFN), TALE nucleases (TALENS), meganucleases, and CRISPR-Cas systems.

### CRISPR-Cas

In one example embodiment, the gene editing system is a CRISPR-Cas system. The CRISPR-Cas systems comprise a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

### NHEJ-Based Editing

In one example embodiment, the CRISPR-Cas system is used to introduce one or more insertions or deletions that restore POU2FA binding to an enhancer that controls expression of COBL11. More than one guide sequence may be selected to insert multiple insertion, deletions, or combination thereof. Likewise, more than one Cas protein type may be used, for example, to maximize targets sites adjacent to different PAMs. In one example embodiment, a guide sequence is selected that directs the CRISPR-Cas system to make one or more insertions or deletions within the enhance region containing a variant that reduces POU2A binding to an enhancer controlling COBL11 expression. In one example embodiment, a guide is selected that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs upstream of a variant that reduces POU2FA binding to an enhancer controlling COBL11 expression. In one example embodiment, a guide sequence is selected to that directs the CRISPR-Cas system to make an insertion 1, 2, 3, 4, 5, 6, 7, 8, 9, 10, 11, 12, 13, 14, 15, 16, 17, 18, 19, 20, 21, 22, 23, 24, or 25 base pairs downstream of a variant that reduces POU2FA binding to an enhancer controlling COBL11 expression. In one example embodiment, a guide sequence is selected to that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of a variant that reduces POU2FA binding to an enhancer controlling COBL11 expression. In one example embodiment, a guide sequence is selected to that directs the CRISPR-Cas system to make a deletion 1, 2, 3, 4, 5, 6, 7, 8, 9, 10, 11, 12, 13, 14, 15, 16, 17, 18, 19, 20, 21, 22, 23, 24, or 25 base pairs downstream of a variant that reduces POU2FA binding to an enhancer controlling COBL11 expression. In one example embodiment, the above insertions and/or deletions are made relative to the rs6712203 variant position.

### HDR Template Based Editing

In one example embodiment, a donor template is provided to replace a genomic sequence comprising one or more variants that reduce COBL11A expression. A donor template may comprise an insertion sequence flanked by two homology regions. The insertion sequence comprises an edited sequence to be inserted in place of the target sequence (e.g. a portion of genomic DNA comprising the one or more variants). The homology regions comprise sequences that are homologous to the genomic DNA strands at the site of the CRISPR-Cas induced double-strand break. Cellular HDR mechanisms then facilitate insertion of the insertion sequence at the site of the DSB.

Accordingly, in certain example embodiments, a donor template and guide sequence are selected to direct excision and replacement of a section of genome DNA comprising a variant that reduces POU2FA binding to an enhancer controlling COBL11 expression with an insertion sequence that edits the one or more variants to a wild-type or non-risk variant. In one example embodiment, the insertion sequence comprises a wild-type or non-risk variant that restores or increases POU2FA binding to the enhancer. In one example embodiment, the insertion sequence encodes a portion of genomic DNA in which the rs6712203 variant is changed from a C to a T.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144 149).

### Class 1 Systems

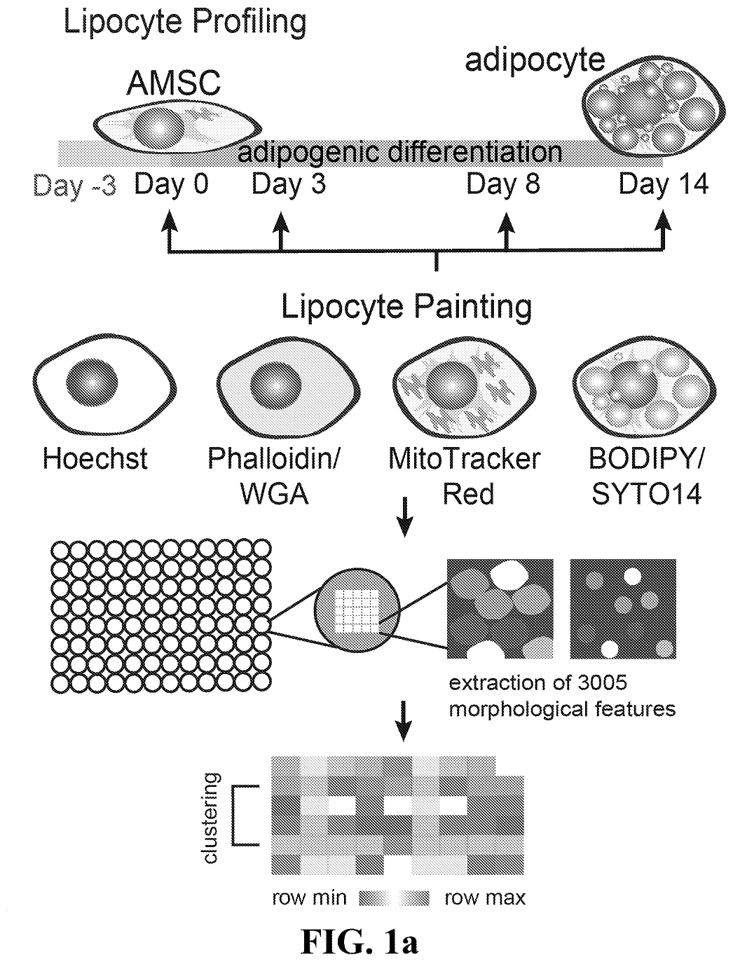

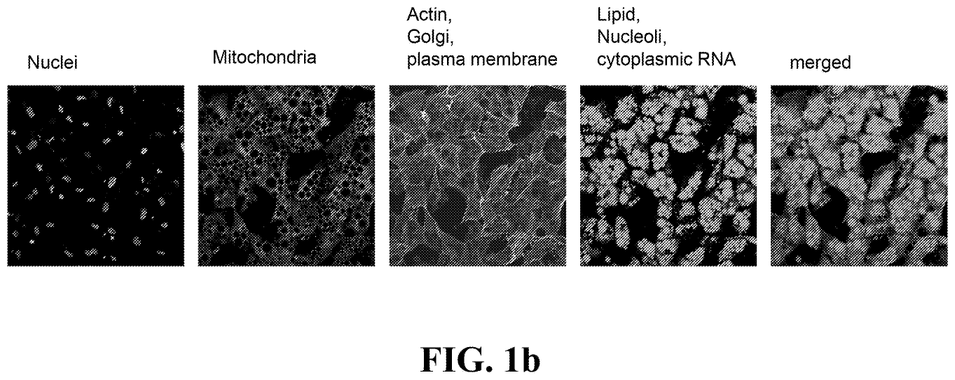

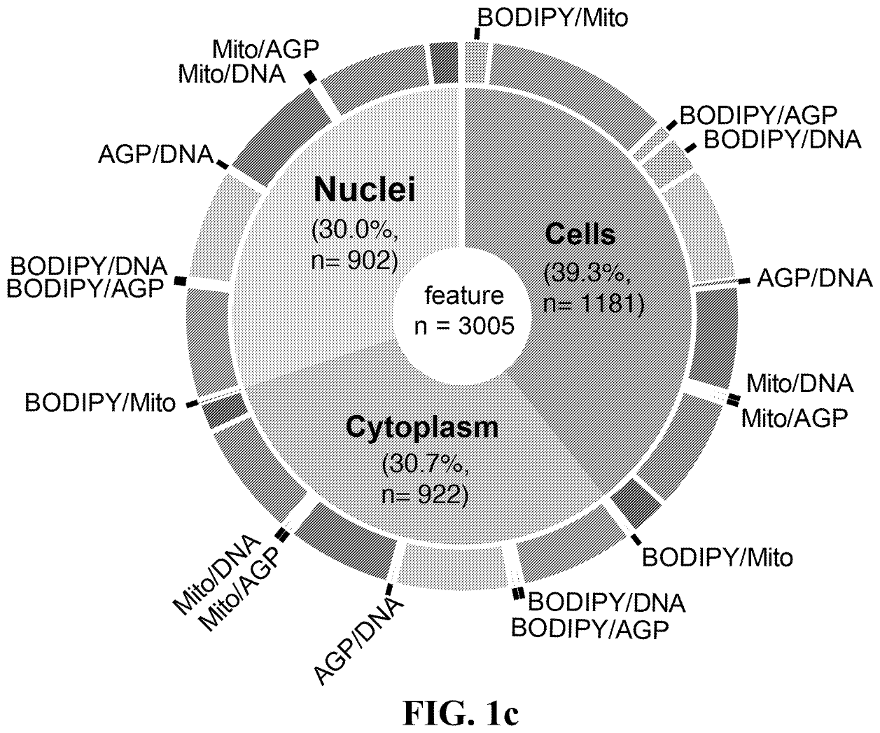

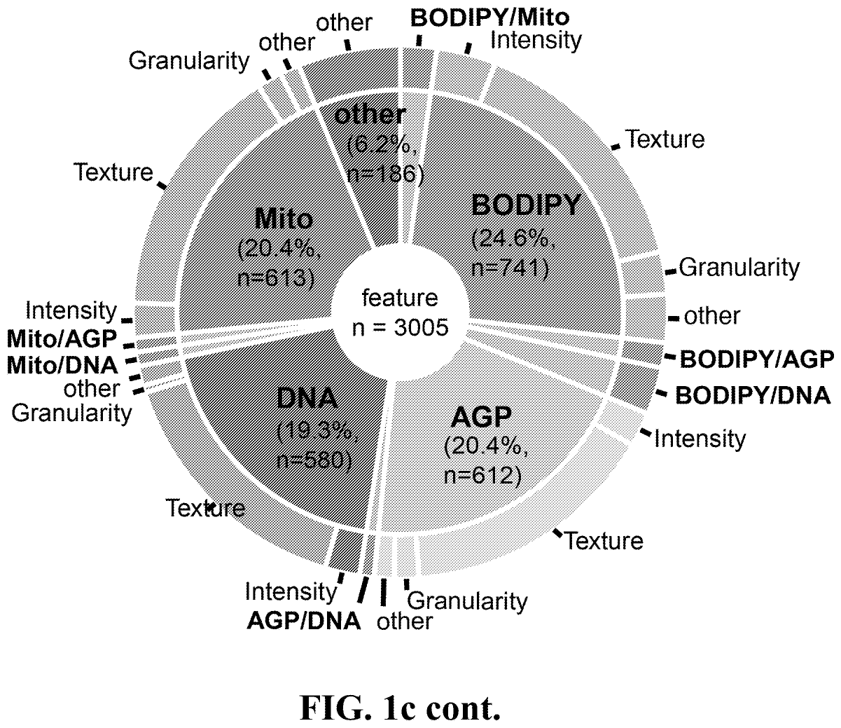

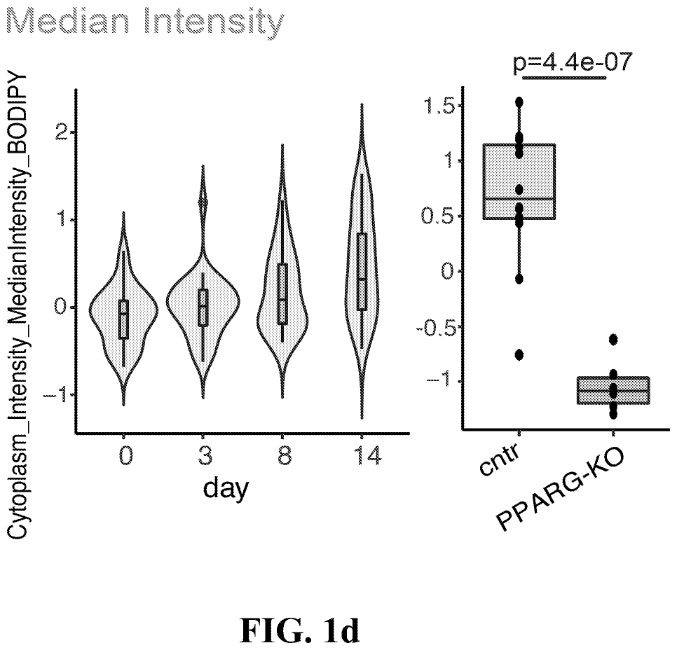

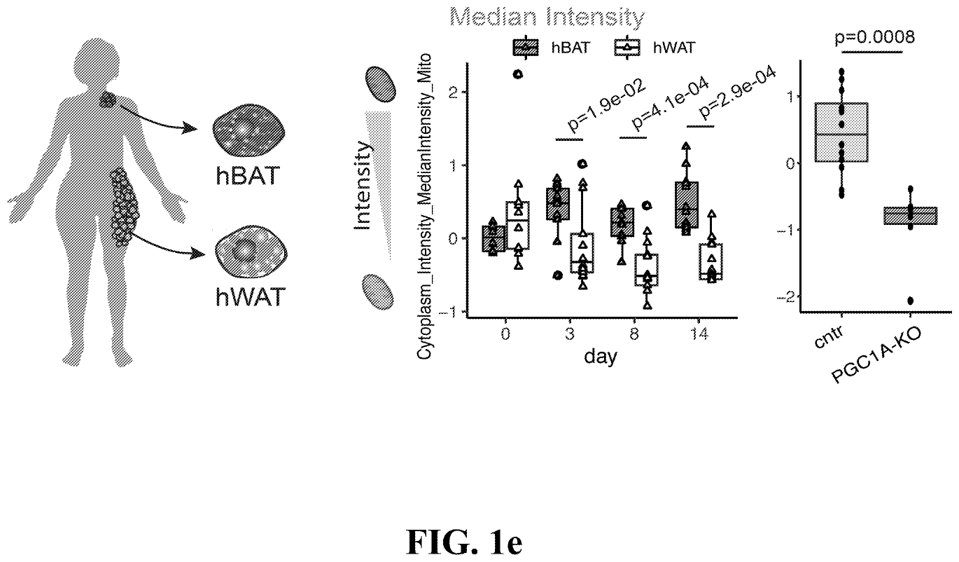

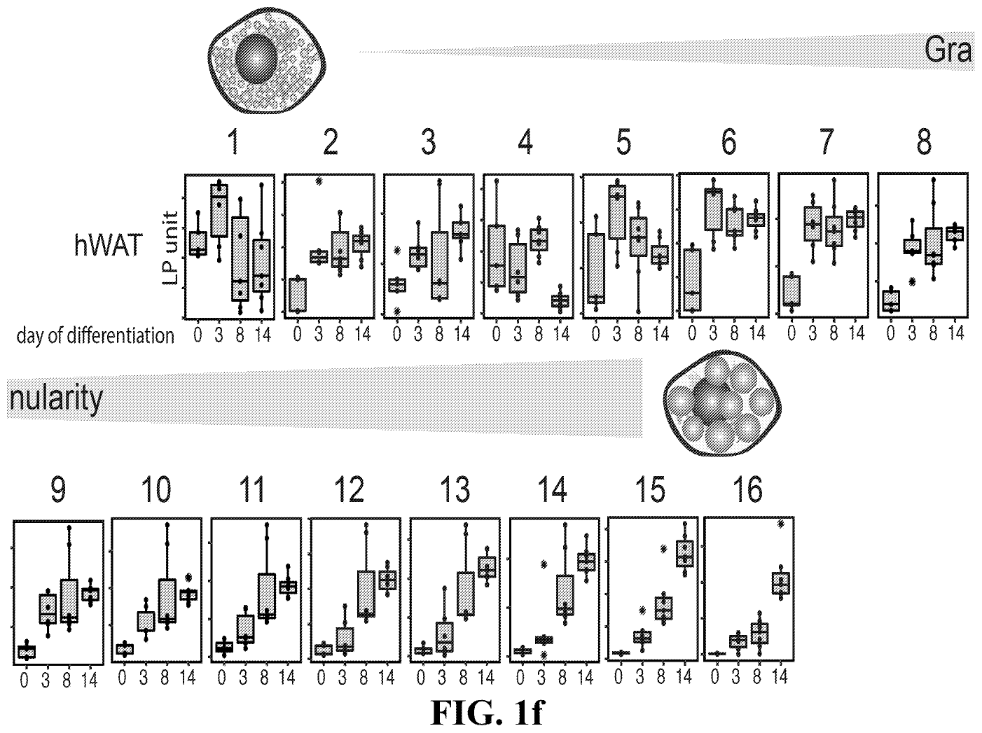

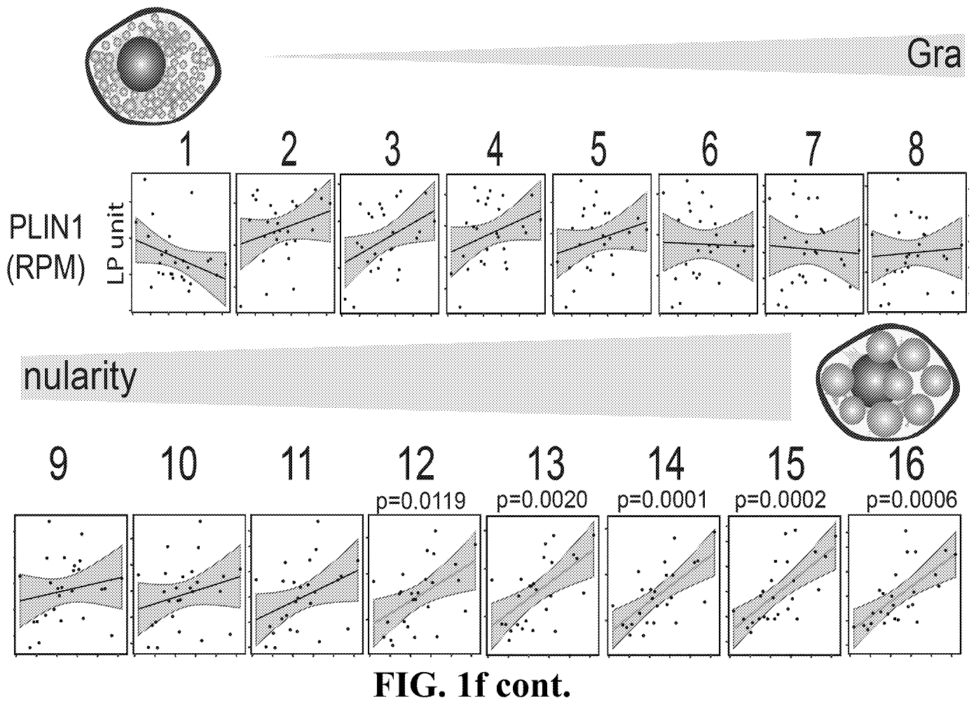

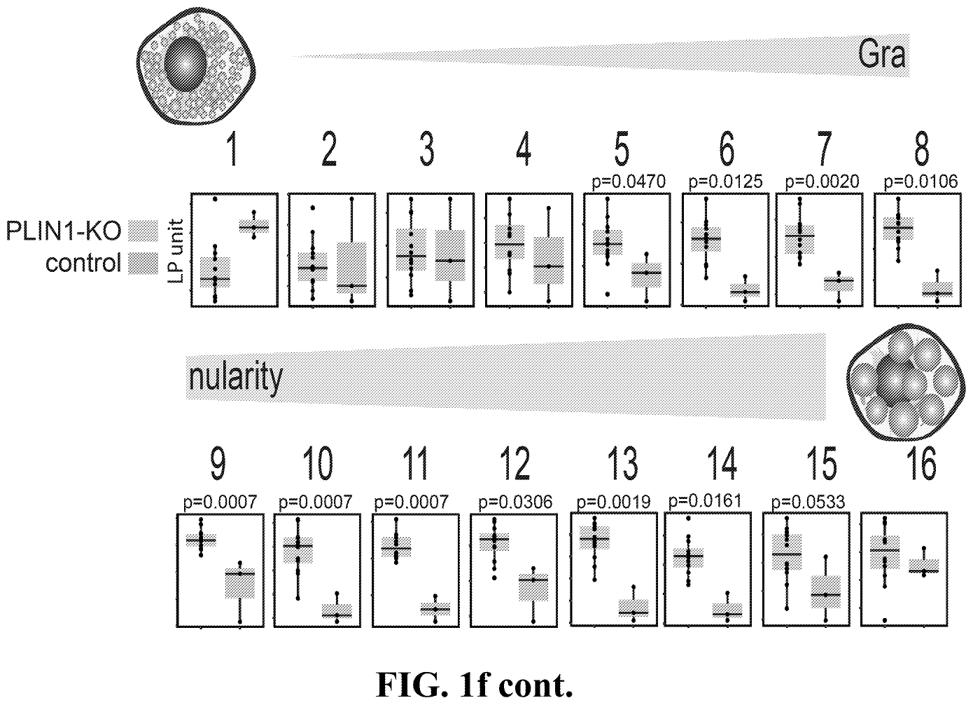

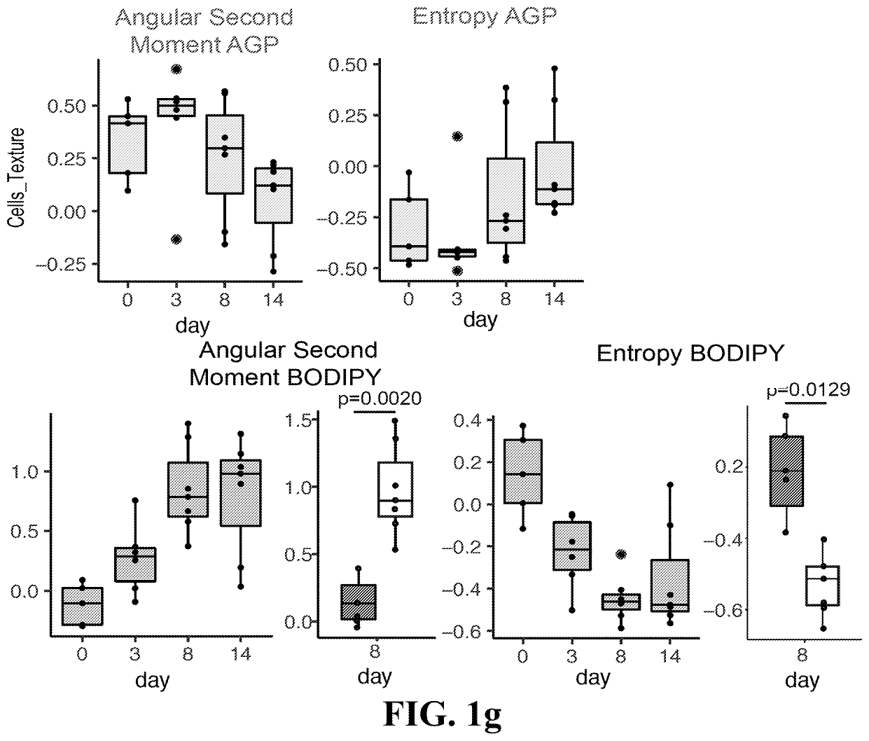

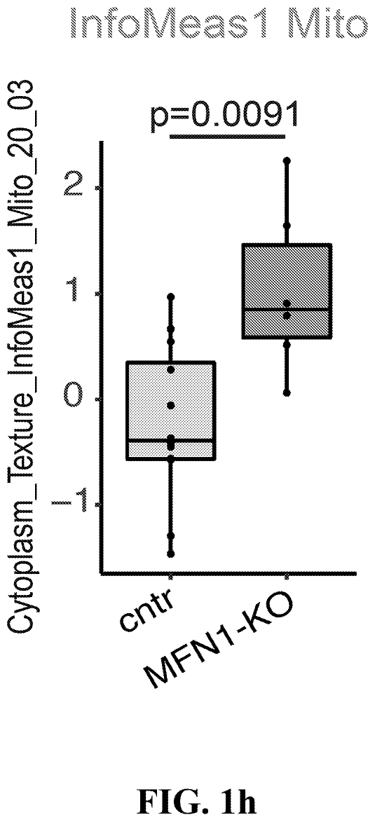

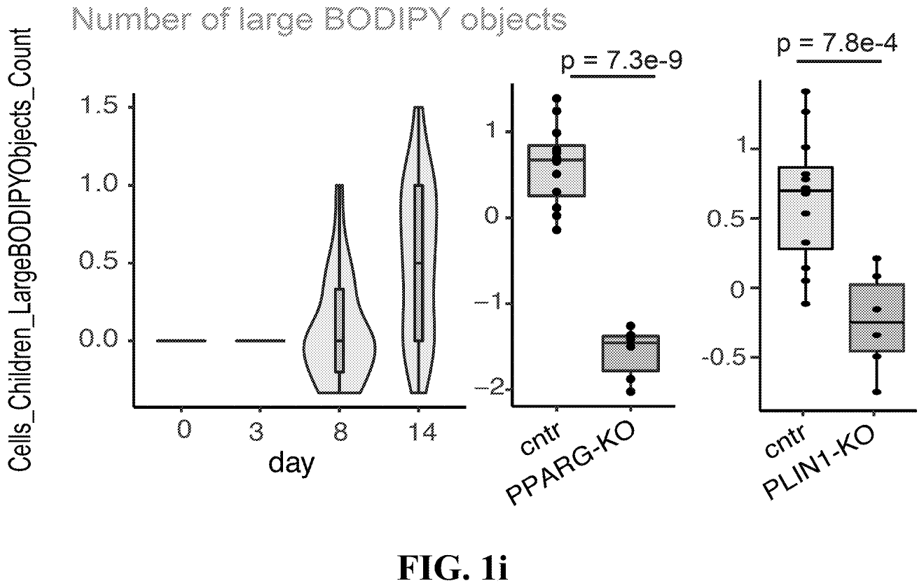

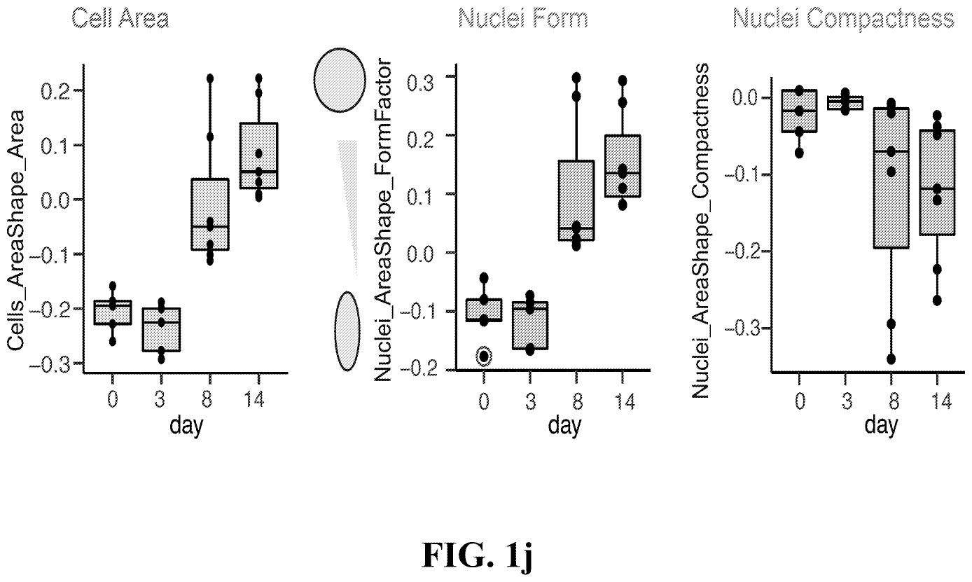

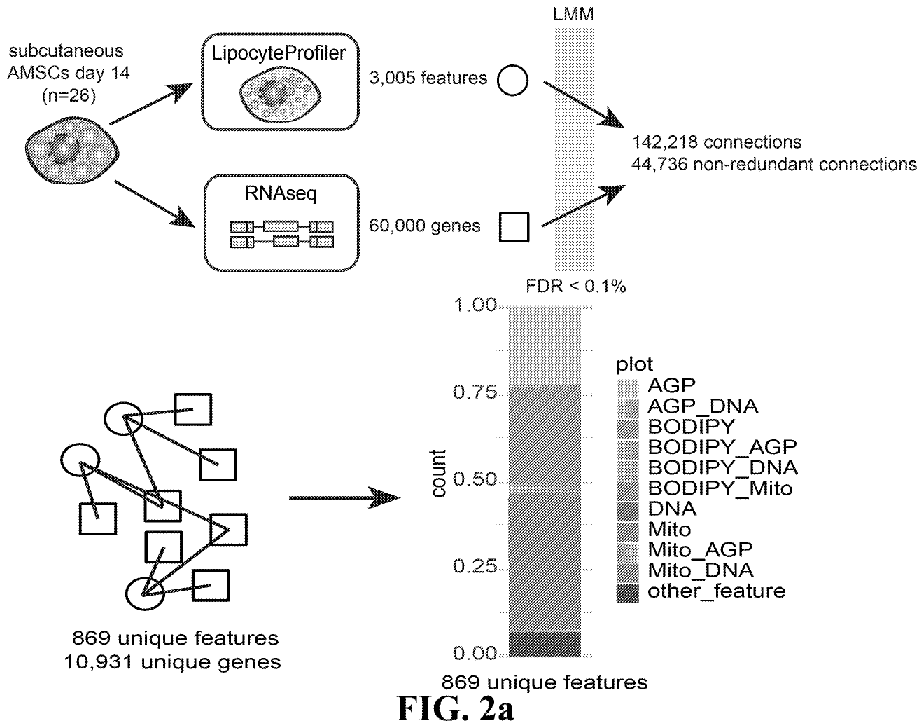

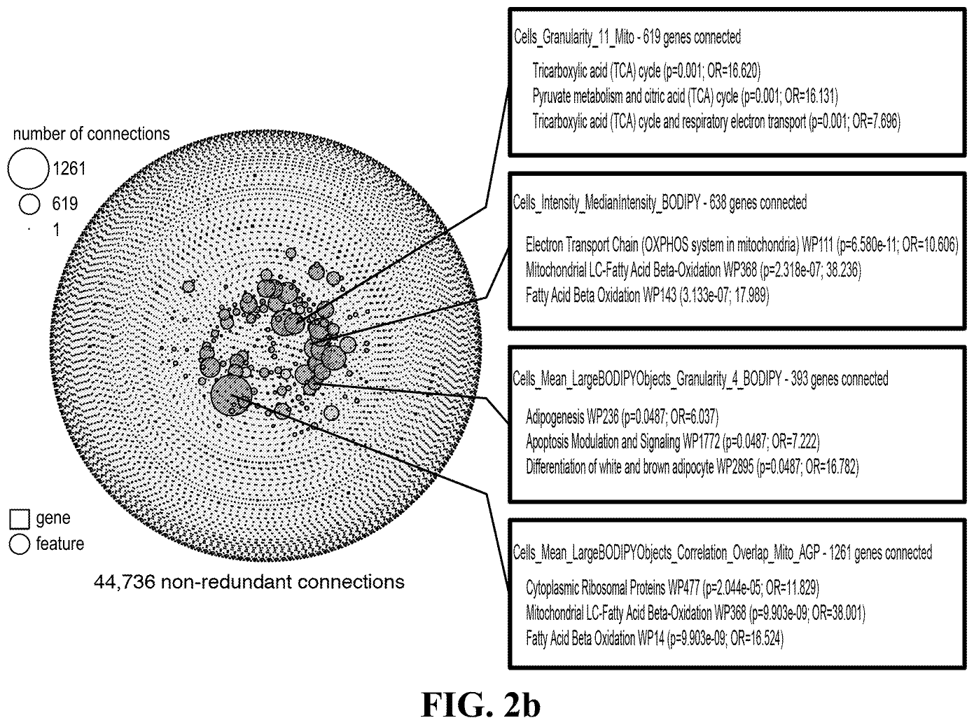

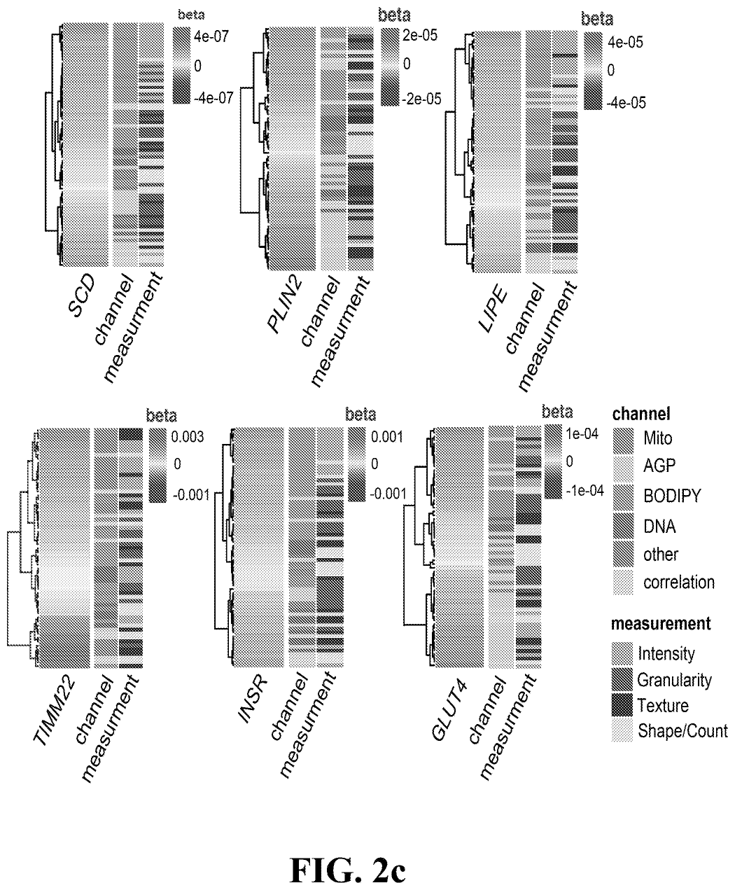

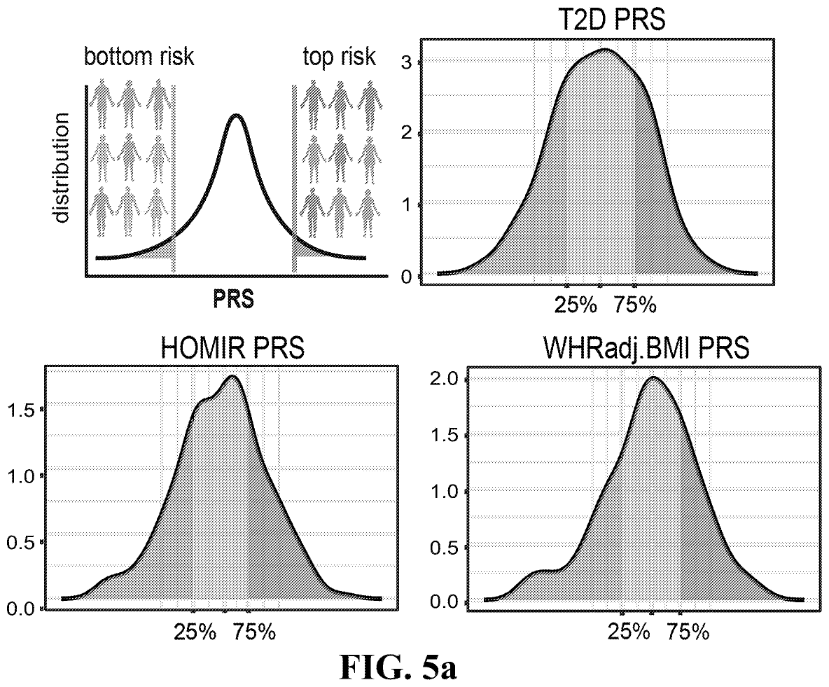

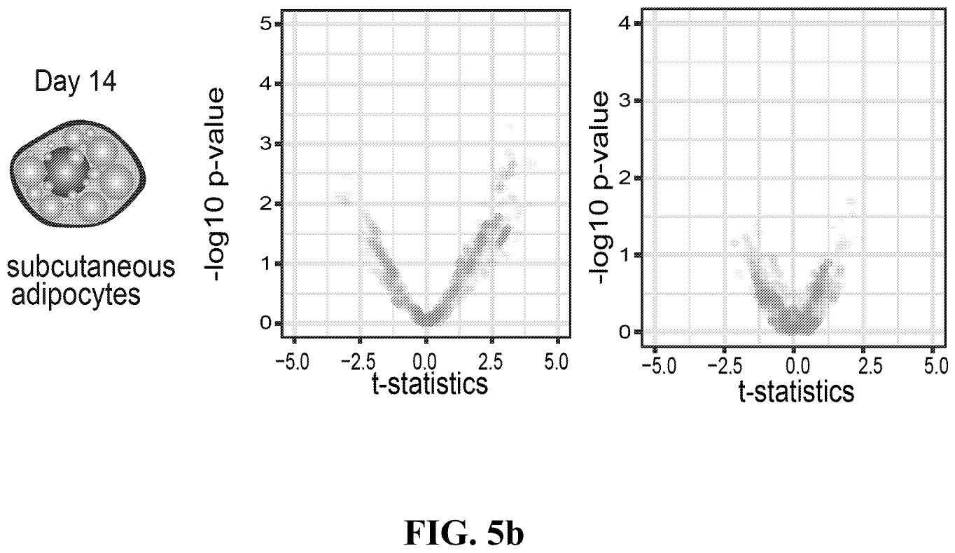

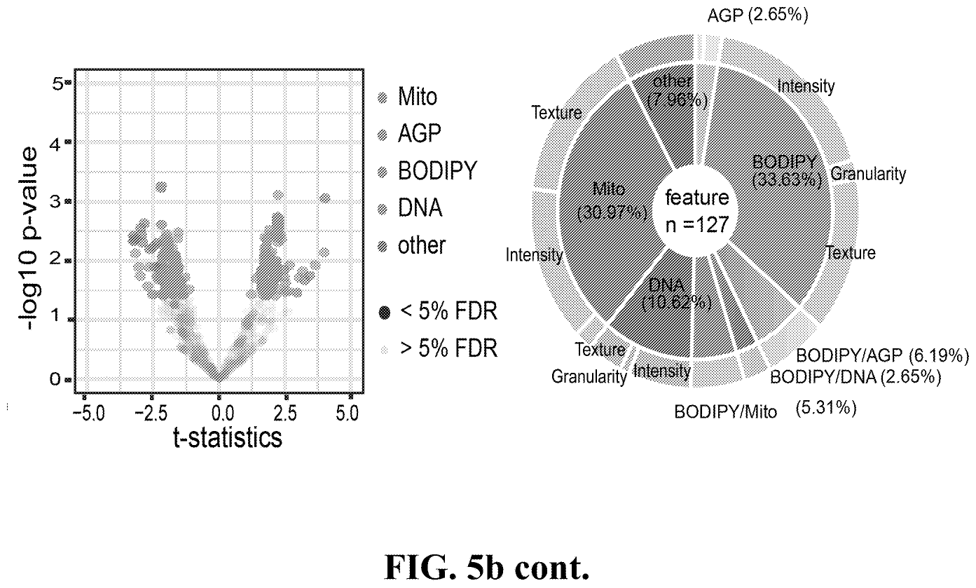

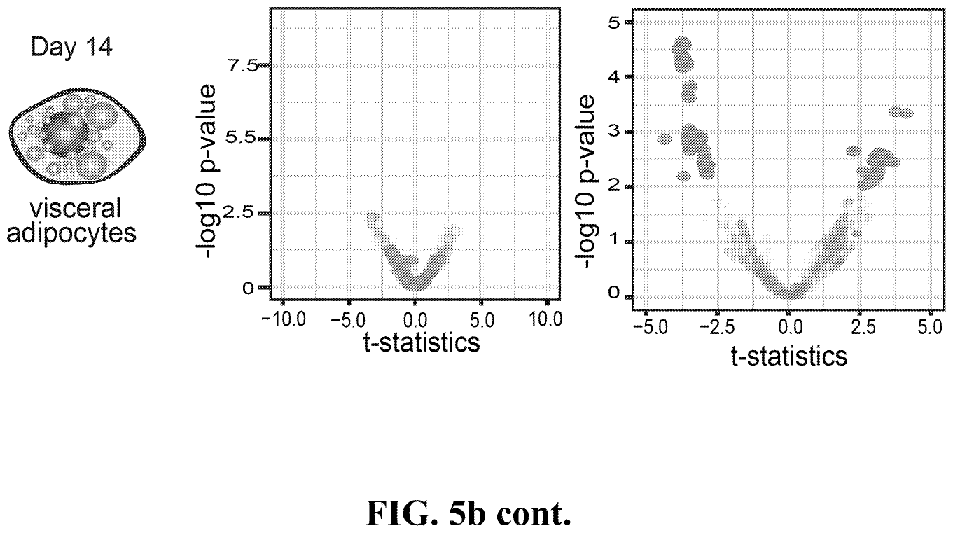

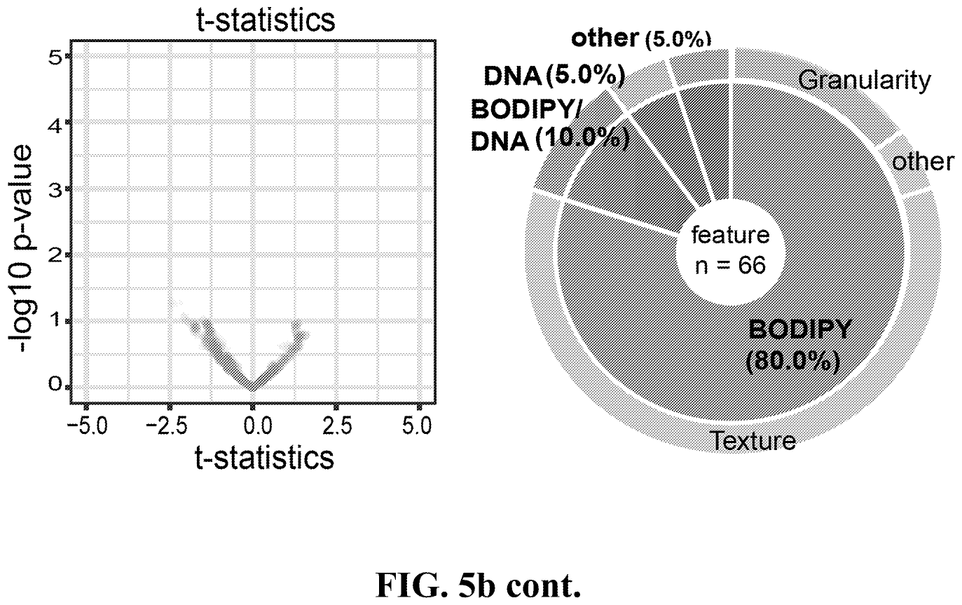

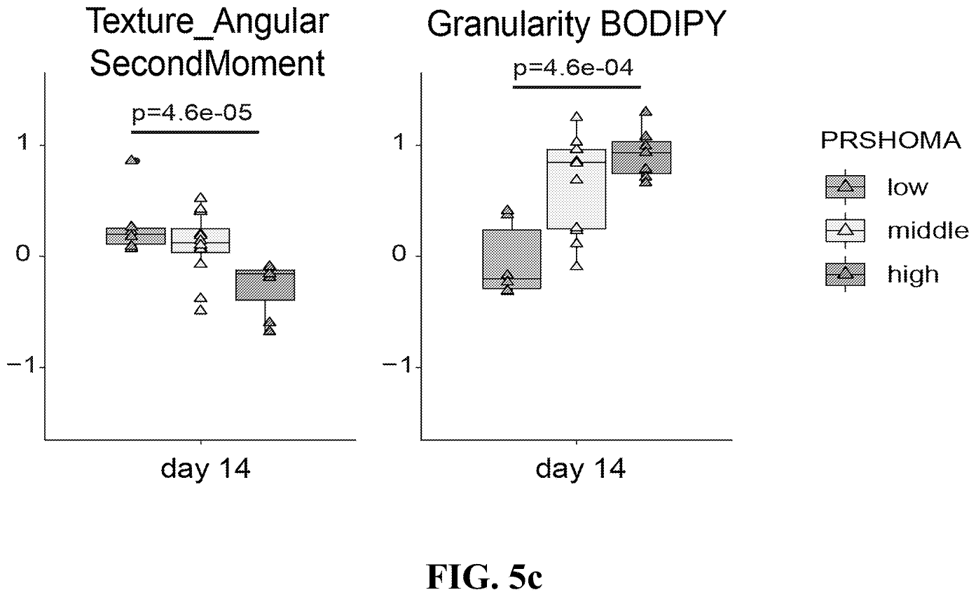

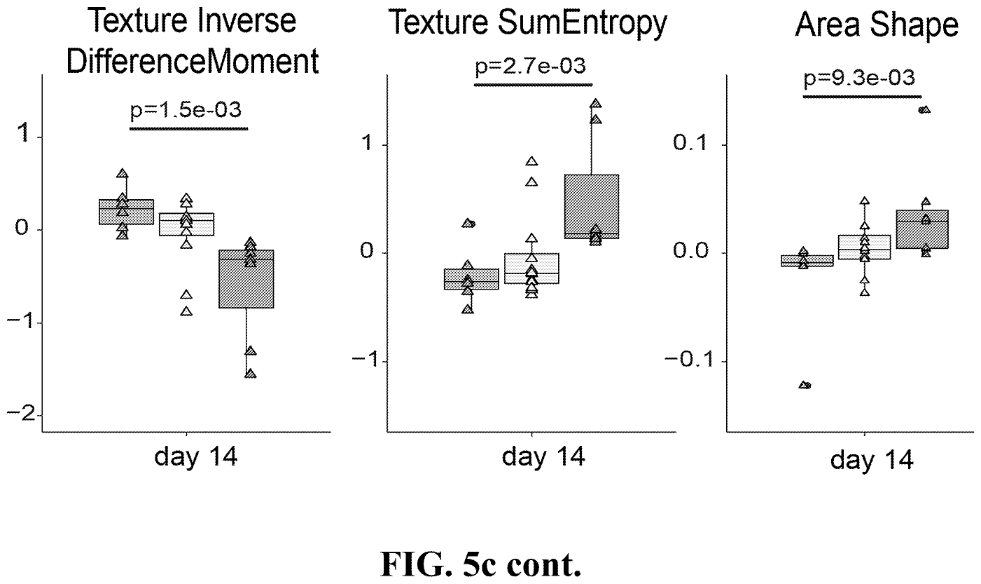

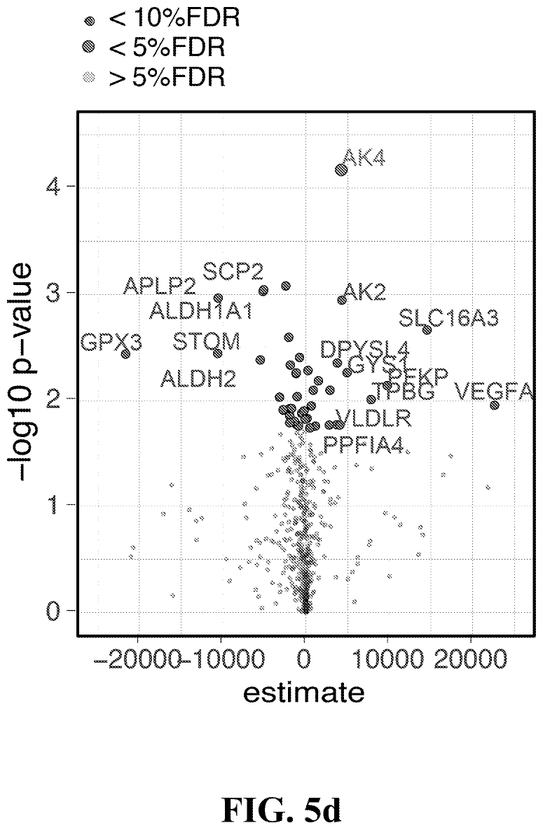

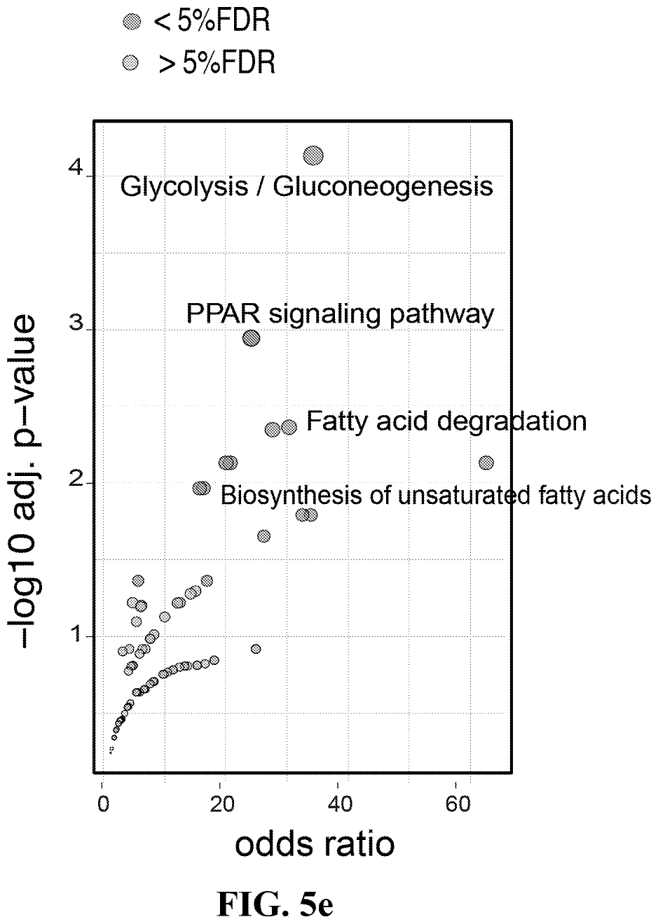

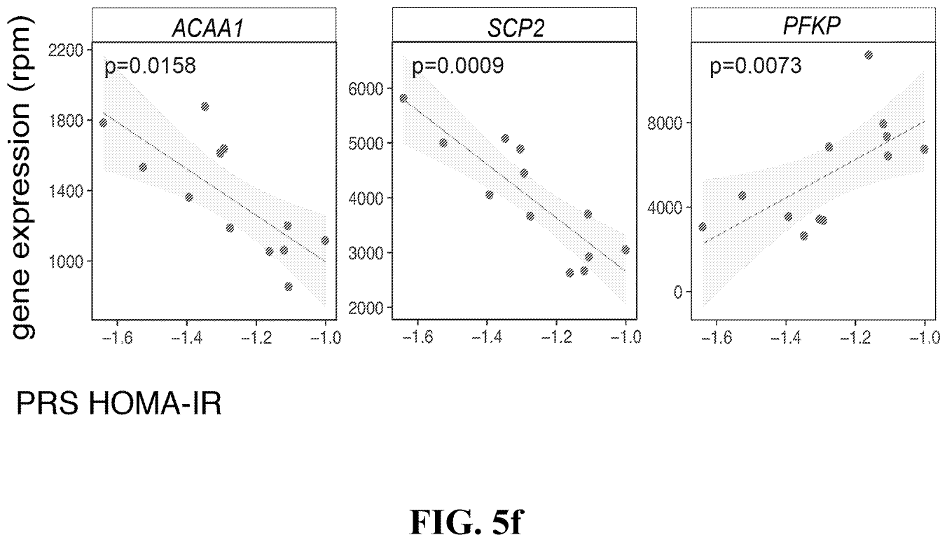

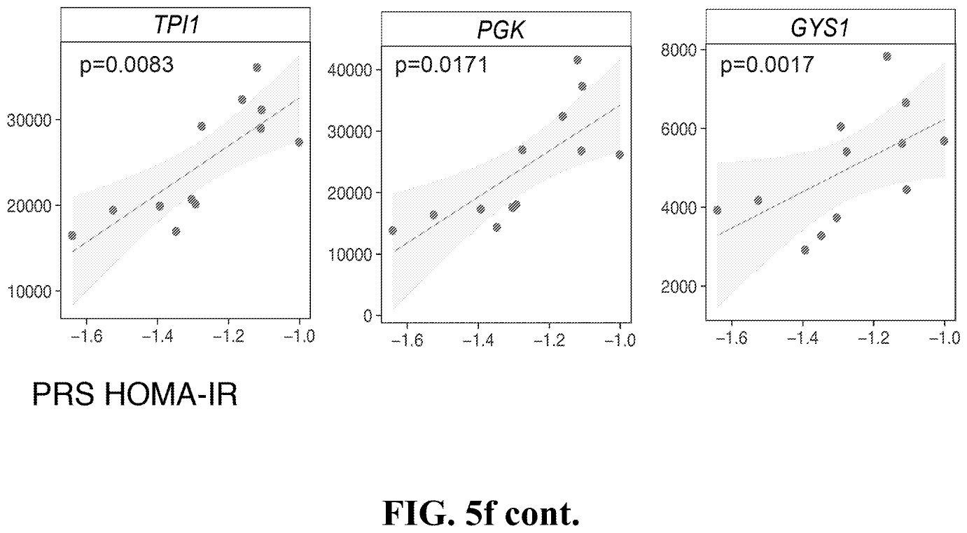

The CRISPR-Cas therapeutic methods disclosed herein may be designed for use with Class 1 CRISPR-Cas systems. In certain example embodiments, the Class 1 system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020)., incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374:20180087, DOI: 10.1098/rstb.2018.0087. In one aspect, Class 1 systems are characterized by the signature protein Cas3. The Cascade in particular Class 1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al, the CRISPR Journal, v. 1, n5, FIG. 5.

### Class 2 Systems

The CRISPR-Cas therapeutic methods disclosed herein may be designed for use with. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1 (V-U3), V-F2, V-F3, V-G, V-II, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a RuvC-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type II CRISPR-Cas system is a II-C1 CRISPR-Cas system. In one example embodiment, the Type II CRISPR-Cas system is a II-C2 CRISPR-Cas system. In some example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2c1), Cas12c (C2c3), Cas12d (CasY), Cas12e (CasX), Cas14, and/or CasΦ.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36 (4) 702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In I some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and P A Carr and G M Church, 2009, Nature Biotechnology 27 (12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracer sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16 (4): 504-517. Table C (from Gleditzsch et al. 2019) below shows several Cas polypeptides and the PAM sequence they recognize.

TABLE C
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, |
| | TAG, and CAC |
| Type I-C | NTTC |
| Type I-E | 5′-AAG-3′ |
| TypeI-F | GG |

In a preferred embodiment, the CRISPR effector protein may recognize a 3′ PAM. In one example embodiment, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523 (7561): 481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155 (Pt. 3): 733-740; Atschul et al. 1990. J. Mol. Biol. 215:403-410; Biswass et al. 2013 RNA Biol. 10:817-827; and Grissa et al. 2007. Nucleic Acid Res. 35: W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16 (4): 504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16 (4): 504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 23) or PKKKRKVEAS (SEQ ID NO:24); the NLS from nucleoplasmin (e.g., the nucleoplasmin bipartite NLS with the sequence KRPAATKKAGQAKKKK (SEQ ID NO: 25)); the c-myc NLS having the amino acid sequence PAAKRVKLD (SEQ ID NO: 26) or RQRRNELKRSP (SEQ ID NO: 27); the hRNPA1 M9 NIS having the sequence NOSSNFGPMKGGNFGGRSSGPYGGGGQYFAKPRNQGGY (SEQ ID NO: 28); the sequence RMRIZFKNKGKDTAELRRRRVEVSVELRKAKKDEQILKRRNV (SEQ ID NO: 29) of the IBB domain from importin-alpha; the sequences VSRKRPRP (SEQ ID NO: 30) and PPKKARED (SEQ ID NO: 31) of the myoma T protein; the sequence PQPKKKPL (SEQ ID NO: 32) of human p53; the sequence SALIKKKKKMAP (SEQ ID NO: 33) of mouse c-abl IV; the sequences DRLRR (SEQ ID NO: 34) and PKQKKRK (SEQ ID NO: 35) of the influenza virus NS1; the sequence RKLKKKIKKL (SEQ ID NO: 36) of the Hepatitis virus delta antigen; the sequence REKKKFLKRR (SEQ ID NO: 37) of the mouse Mx1 protein; the sequence KRKGDEVDGVDEVAKKKSKK (SEQ ID NO: 37) of the human poly(ADP-ribose) polymerase; and the sequence RKCLQAGMNLEARKTKK (SEQ ID NO: 39) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NIS at the carboxy terminus). When more than one NIS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

### Zinc Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain (e.g., activator domain). The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALENs

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39: e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or ₃₄ or ₃₅, where the subscript indicates the amino acid position and X represents any amino acid. X₁₂X₁₃ indicate the RVDs. In some polypeptide monomers, the variable amino acid at position 13 is missing or absent and in such monomers, the RVD) consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or ₃₄ or ₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD) of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011). each of which is incorporated herein by reference in its entirety.

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in one example embodiment, the TALE polypeptides described herein further comprise an N-terminal capping region and/or a C-terminal capping region.

An exemplary amino acid sequence of a N-terminal capping region is:

(SEQ ID NO: 40)
M D P I R S R T P S P A R E L L S G P Q P D G V
Q P T A D R G V S P P A G G P L D G L P A R R T
M S R T R L P S P P A P S P A F S A D S F S D L
L R Q F D P S L F N T S L F D S L P P F G A H H
T E A A T G E W D E V Q S G L R A A D A P P P T
M R V A V T A A R P P R A K P A P R R R A A Q P
S D A S P A A Q V D L R T L G Y S Q Q Q Q E K I
K P K V R S T V A Q H H E A L V G H G F T H A H
I V A L S Q H P A A L G T V A V K Y Q D M I A A
L P E A T H E A I V G V G K Q W S G A R A L E A
L L T V A G E L R G P P L Q L D T G Q L L K I A
K R G G V T A V E A V H A W R N A L T G A P L N

An exemplary amino acid sequence of a C-terminal capping region is:

(SEQ ID NO: 41)
R P A L E S I V A Q L S R P D P A L A A L T N D
H L V A L A C L G G R P A L D A V K K G L P H A
P A L I K R T N R R I P E R T S H R V A D H A Q
V V R V L G F F Q C H S H P A Q A F D D A M T Q
F G M S R H G L L Q L F R R V G V T E L E A R S
G T L P P A S Q R W D R I L Q A S G M K R A K P
S P T S T Q T P D Q A S L H A F A D S L E R D L
D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in one example embodiment, fragments of the N-terminal and/or C-terminal capping regions are included in the TALE polypeptides described herein.

In one example embodiment, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In another example embodiment, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In one example embodiment, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In one example embodiment, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a/F array targets three DNA bases. A customized array of individual zinc finger domains is assembled into a/F protein (ZFP).

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Engineered Transcriptional Activators (CRISPRa)

In one example embodiment, a programmable nuclease system is used to recruit an activator protein to the COBLL1 gene in order to enhance expression. In one example embodiment, the activator protein is recruited to the enhancer region of the COBLL1 gene. In another example embodiment, the nuclease system is programmed to bind a sequence variant responsible for decreased COBLL1 expression. In another example embodiment, the nuclease system is recruited to a POU2F2 binding site comprising a mutation that decreases or eliminates binding by POU2F2. In a preferred embodiment, the mutation is rs6712203. In another embodiment, the mutation is rs6712203 and the nuclease system is recruited within 20 base pairs surrounding it. In another example embodiment, the nuclease system is recruited to an enhancer possessing the variant. For example, if a subject comprises a variant that prevents binding of a transcription factor to an enhancer controlling expression of COBLL1, a catalytically inactive Cas protein (“dCas”) fused to an activator can be used to recruit that activator protein to the mutated sequence. Accordingly, a guide sequence is designed to direct binding of the dCas-activator fusion such that the activator can interact with the target genomic region and induce COBLL1 expression. In one example embodiment, the guide is designed to bind within 10, 20, 30, 40, 50, 60, 70, 80, 90, 100 or up to 500 base pairs of the variant nucleotide. In one example embodiment, a CRISPR guide sequence includes the specific variant nucleotide. In one example embodiment, POU2F2 or the activation domain thereof is recruited to the COBLL1 enhancer. The Cas protein used may be any of the Cas proteins disclosed above. In one example protein, the Cas protein is a dCas9.

In one embodiment, the programmable nuclease system is a CRISPRa system (see, e.g., US20180057810A1; and Konermann et al. “Genome-scale transcriptional activation by an engineered CRISPR-Cas9 complex” Nature. 2014 Dec. 10. doi: 10.1038/nature14136). Numerous genetic variants associated with disease phenotypes are found to be in non-coding region of the genome, and frequently coincide with transcription factor (TF) binding sites and non-coding RNA genes. In one embodiment, a CRISPR system may be used to activate gene transcription. A nuclease-dead RNA-guided DNA binding domain, dCas9, tethered to transcriptional activator domains that promote gene activation (e.g., p65) may be used for “CRISPRa” that activates transcription. In one example embodiment, for use of dCas9 as an activator (CRISPRa), a guide RNA is engineered to carry RNA binding motifs (e.g., MS2) that recruit effector domains fused to RNA-motif binding proteins, increasing transcription. A key dendritic cell molecule, p65, may be used as a signal amplifier, but is not required.

In certain embodiments, one or more activator domains are recruited. In one example embodiment, the activation domain is linked to the CRISPR enzyme. In another example embodiment, the guide sequence includes aptamer sequences that bind to adaptor proteins fused to an activation domain. In general, the positioning of the one or more activator domains on the inactivated CRISPR enzyme or CRISPR complex is one which allows for correct spatial orientation for the activator domain to affect the target with the attributed functional effect. For example, the transcription activator is placed in a spatial orientation which allows it to affect the transcription of the target. This may include positions other than the N-/C-terminus of the CRISPR enzyme.

In another example embodiment, a zinc finger system is used to recruit an activation domain to the COBLL1 gene. In one example embodiment, the activation domain is linked to the zinc finger system. In general, the positioning of the one or more activator domains on the zinc finger system is one which allows for correct spatial orientation for the activator domain to affect the target with the attributed functional effect.

In another example embodiment, a TALE system is used to recruit an activation domain to the COBLL1 gene. In one example embodiment, the activation domain is linked to the TALE system. In general, the positioning of the one or more activator domains on the TALE system is one which allows for correct spatial orientation for the activator domain to affect the target with the attributed functional effect. For example, the transcription activator is placed in a spatial orientation which allows it to affect the transcription of the target.

In another example embodiment, a meganuclease system is used to recruit an activation domain to the COBLL1 gene. In one example embodiment, the activation domain is linked to the meganuclease system. In general, the positioning of the one or more activator domains on the inactivated meganuclease system is one which allows for correct spatial orientation for the activator domain to affect the target with the attributed functional effect. For example, the transcription activator is placed in a spatial orientation which allows it to affect the transcription of the target.

### Base Editing

In one example embodiment, a method of treating subjects suffering from, or at risk of developing, T2D) comprises administering a base editing system that corrects one or more variants associated with decreased expression or activity of COBL11 in adipocyte and/or adipocyte progenitors. A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas12a or Cas12b polypeptide. The nucleobase deaminase may be cytosine base editor (CBE) or adenosine base editors (ABEs). CBEs convert C⋅G base pairs into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Example base editing systems are disclosed in Rees and Liu. 2018. Nat. Rev. Genet. 19 (12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

The editing window of a base editing system may range over a 5-8 nucleotide window, depending on the base editing system used. Id. Accordingly, given the base editing system used, a guide sequence may be selected to direct the base editing system to convert a base or base pair of one or more variants resulting in reduced POU2FA binding to an enhancer controlling COBL11 expression to a wild-type or non-risk variant. In one example embodiment, the variant is rs6712203. Accordingly, in one example embodiment, the base editing system comprises a CBE capable of editing the C of rs6712203 to a T. In one embodiment, the variant is rs12454712. Accordingly, in one example embodiment, the base editing system comprises a CBE capable of editing the T of rs12454712 to a C.

### ARCUS Based Editing

In one example embodiment, a method of treating subjects suffering from, or at risk of developing, T2D comprises administering an ARCUS base editing system. Exemplary methods for using ARCUS can be found in U.S. Pat. No. 10,851,358, US Publication No. 2020-0239544, and WIPO Publication No. 2020/206231 which are incorporated herein by reference.

### Prime Editing

In one example embodiment, a method of treating subjects suffering from, or at risk of developing, T2D comprises administering a prime editing system that corrects one or more variants associated with decreased expression or activity of COBL11 in adipocyte and/or adipocyte progenitors. In one example embodiment, a method of treating subjects suffering from, or at risk of developing, lipodystrophy comprises administering a prime editing system that corrects one or more variants associated with decreased expression or activity of BCL2 in skeletal muscle or ASMCs and/or KDSR in ASMCs. In an example embodiment, a method of treating subjects suffering from, or at risk of developing, lipodystrophy comprises administering a prime editing system that corrects one or more variants associated with increased expression or activity of VPS4B in ASMCs. In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g. genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place. In on example embodiment, a pegRNA is configured to direct the prime editing system to convert a single base or base pair of the one or more variants associated with reduced COBL11 expression to a wild-type or non-risk variant. In one example embodiment, a pegRNA is configured to direct the prime editing system to convert a single base or base pair of one or more variants associated with reduced POU2FA binding to an enhancer controlling COBL11 expression such that POU2FA binding affinity to the enhance. In another example embodiment, a pegRNA is configured to direct the prime editing system to convert to C of rs6712203 to a T. In another example embodiment, a pegRNA is configured to direct the prime editing system to excise a portion of genomic DNA comprising one or more variants associated with reduced expression of COBL11 with a sequence that replaces the one or more variants with a wild-type or non-risk variant. In another example embodiment, a pegRNA is configured to direct the prime editing system to excise a portion of genomic DNA comprising one or more variants that reduce POU2FA binding to an enhancer controlling COBL11 expression such that the binding affinity of POU2FA is restored. In one example embodiment, the one or more variants comprise rs6712203. Accordingly, in one example embodiment, a pegRNA is configured to the prime editing system to excise a portion of genomic DNA comprising rs6712203 and replace with a polynucleotide sequence in which the C of rs6712203 is replaced with a T.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576:149-157, particularly at pg. 3, FIG. 2a-2b, and Extended Data FIGS. 5a-c.

### CRISPR Associated Transposases (CAST)

In one example embodiment, a method of treating subject suffering from, or at risk of developing, T2D comprises administering a CAST system that replaces a genomic region comprising one or more variants associated with decreased expression or activity of COBL11 in adipocyte and/or adipocyte progenitors with a polynucleotide sequence comprising a wild type sequence or non-risk variant. In one example embodiment, a CAST system is used to replace all or a portion of an enhancer controlling COBL11 expression and comprising one or more variants that reduce POU2FA binding to the enhancer. In one example embodiment, a CAST system is used to replace a portion of genomic DNA comprising the rs6712203 variant with a sequence that replaces the C of rs6712203 with a T.

In one example embodiment, a method of treating subject suffering from, or at risk of developing, lipodystrophy comprises administering a CAST system that replaces a genomic region comprising one or more variants associated with decreased expression or activity of BCL2 in ASMCs or skeletal muscle and/or KDSR in ASMCs with a polynucleotide sequence comprising a wild type sequence or non-risk variant. In one example embodiment, a method of treating subject suffering from, or at risk of developing, lipodystrophy comprises administering a CAST system that replaces a genomic region comprising one or more variants associated with increased expression or activity of VPS4B in ASMCs. In one example embodiment, a CAST system is used to replace a portion of genomic DNA comprising the rs12454712 variant with a sequence that replaces the T of rs12454712 with a C.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g. genomic DNA). The guide molecule is capable of forming a CRISPR-Cas complex with the Cas polypeptide, and can be programmed to direct the entire CAST complex such that the transposase is positioned to insert the donor sequence at the target site on the target polynucleotide. For multimeric transposase, only those transposases needed for recognition of the donor construct and transposition of the donor sequence into the target polypeptide may be required. The Cas may be naturally catalytically inactive or engineered to be catalytically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-If Cas polypeptide. In one example embodiment, the Cas polypeptide may comprise a cas6, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type If-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-1b Cas polypeptide. In one example embodiment, the Cas polypeptide may comprise a cas6, a cas7, and a cas8b (e.g. a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transposon complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO/2021/041922 which is incorporated herein by reference.

In one example embodiment, the CAST comprise a catalytically inactive Type II Cas polypeptide (e.g. dCas9) fused to one or more polypeptides of a Tn5 transposase. In another example embodiment, the CAST system comprises a catalytically inactive Type II Cas polypeptide (e.g. dCas9) fused to a piggyback transposase

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

Delivery mechanisms for CAST systems includes those discussed above for CRISPR-Cas systems.

### Adoptive Cell Transfer (ACT)

In one example embodiment, a subject at risk for, or suffering from, Type-2 Diabetes (T2D)) due to decreased COBLL1 expression or activity or aberrant actin remodeling is treated by transplanting AMSCs having normal function to adipose tissue in the subject (ACT). As used herein, “transplant” refers to transferring cells to a subject to replace or supplement cells or tissue causing disease and can be used interchangeably with “ACT”. The AMSCs may be obtained from a donor (allogenic) or obtained from the subject (autologous) and modified using gene therapy to have normal function when differentiated into adipocytes. As used herein, “ACT”, “adoptive cell therapy” and “adoptive cell transfer” may be used interchangeably. In another example embodiment, Adoptive cell therapy (ACT) can refer to the transfer of cells to a patient with the goal of transferring the functionality and characteristics into the new host by engraftment of the cells. As used herein, the terms “engraft” or “engraftment” refers to the process of cell incorporation into a tissue of interest in vivo through contact with existing cells of the tissue. Adoptive cell therapy (ACT) can refer to the transfer of cells back into the same patient or into a new recipient host with the goal of transferring the functionality and characteristics into the new host (e.g., adipocyte function). In another example embodiment, use of autologous cells helps the subject by minimizing graft-versus-host disease (GVHD). In another example embodiment, allogenic AMSCs can be transferred to a subject, as AMSCs are hypoimmunogenic. In another example embodiment, allogenic cells can be edited to reduce alloreactivity and prevent GVHD. Thus, use of allogenic cells allows for cells to be obtained from healthy donors and prepared for use in patients as opposed to preparing autologous cells from a patient after diagnosis. In another example embodiment, gene therapy as described herein can be used to modify cells ex vivo before ACT. In another example embodiment, a programmable nuclease is used to enhance expression of the endogenous COBLL1 gene. In another example embodiment, a polynucleotide sequence encoding COBLL1 is transferred to cells. In another example embodiment, genome editing is used to repair expression of the endogenous COBLL1 gene.

In another example embodiment, a programmable nuclease is used to enhance expression of the endogenous BCL2 gene. In another example embodiment, a polynucleotide sequence encoding BCL2 is transferred to cells. In another example embodiment, genome editing is used to repair expression of the endogenous BCL2 gene. In another example embodiment, a programmable nuclease is used to enhance expression of the endogenous KDSR gene. In another example embodiment, a polynucleotide sequence encoding KDSR is transferred to cells. In another example embodiment, genome editing is used to repair expression of the endogenous KDSR gene. In another example embodiment, a programmable nuclease is used to reduce expression of the endogenous VPS4B gene. In another example embodiment, genome editing is used to repair expression of the endogenous VPS4B gene. In another example embodiment, a programmable nuclease is used to enhance expression of the endogenous VPS4B gene. In another example embodiment, the modified cells can be implanted in the human or animal body to obtain the desired therapeutic effect.

### Adipose Derived Mesenchymal Stem Cells

Mesenchymal stem cells are multipotent stromal cells that can differentiate into a variety of cell types, including osteoblasts (bone cells), chondrocytes (cartilage cells), myocytes (muscle cells) and adipocytes, which are fat cells that give rise to marrow adipose tissue. The bone marrow (BM) stroma contains a heterogeneous population of cells, including endothelial cells, fibroblasts, adipocytes and osteogenic cells, and it was initially thought to function primarily as a structural framework upon which hematopoiesis occurs. However, it turns out that at least two distinct stem cell populations reside in the bone marrow of many mammalian species: hematopoietic stem cells (HSCs) and mesenchymal stem cells (MSCs), with the latter responsible for the maintenance of the non-hematopoietic bone marrow cells. MSCs, also termed multipotent marrow stromal cells or mesenchymal stromal cells, are a heterogeneous population of plastic-adherent, fibroblast-like cells, which can self-renew and differentiate into bone, adipose and cartilage tissue in culture. Single cell suspensions of BM stroma can generate colonies of adherent fibroblast-like cells in vitro. These colony-forming unit fibroblasts (CFU-Fs) are capable of osteogenic differentiation and provide evidence for a clonogenic precursor for cells of the bone lineage. Functional in vitro characterization of the stromal compartment has also revealed its importance in regulating the proliferation, differentiation and survival of HSCs. CFU-F initiating cells in vivo have been shown to be quiescent, existing at a low frequency in human bone marrow.

Although MSCs are traditionally isolated from bone marrow, cells with MSC-like characteristics have been isolated from a variety of fetal, neonatal and adult tissues, including cord blood, peripheral blood, fetal liver and lung, adipose tissue, compact bone, dental pulp, dermis, human islet, adult brain, skeletal muscle, amniotic fluid, synovium, and the circulatory system. There is evidence indicating a perivascular location for these MSC-like cells in all tissues, implying that all MSCs are pericytes that closely encircle endothelial cells in capillaries and microvessels in multiple organs. Pericytes are thought to stabilize blood vessels, contribute to tissue homeostasis under physiological conditions, and play an active role in response to focal tissue injury through the release of bioactive molecules with trophic and immunomodulatory properties. Pericytes and adventitial cells also natively express mesenchymal markers and share similar gene expression profiles as well as developmental and differentiation potential with mesenchymal cells. Pericytes may represent a subpopulation of the total pool of assayable MSCs at least within the bone marrow.

AMSCs can be collected from a subject or donor and can be maintained and expanded in culture for long periods of time without losing their differentiation capacity (see, e.g., Mazini, et al. “Regenerative Capacity of Adipose Derived Stem Cells (ADSCs), Comparison with Mesenchymal Stem Cells (MSCs).” International journal of molecular sciences vol. 20, 10 2523. 22 May. 2019, doi: 10.3390/ijms20102523; and Mazini L, Ezzoubi M, Malka G. Overview of current adipose-derived stem cell (ADSCs) processing involved in therapeutic advancements: flow chart and regulation updates before and after COVID-19. Stem Cell Res Ther. 2021; 12 (1): 1). In another example embodiment, AMSCs are isolated from the subcutaneous adipose tissue (see, e.g., Palumbo, et al. In vitro evaluation of different methods of handling human liposuction aspirate and their effect on adipocytes and adipose derived stem cells. J Cell Physiol. 2015; 230 (8): 1974-1981), which allows for them to be rapidly acquired in large numbers and with a high cellular activity. AMSCs are found in abundant quantities and they are harvested by a minimally invasive procedure, can differentiate into multiple cell lineages in a regulatory and reproducible manner and they are safely transplanted at the both autologous and allogeneic setting (see, e.g., Mazini, et al., 2019). Commercial kits for collection and separation of the stromal vascular fraction (SVF) to isolate AMSCs are available (see, e.g., Mazini, et al., 2019, Table 1). AMSC differentiation into adipocytes is well established and adipose tissue regeneration can be performed in vivo (see, e.g., Tsuji W, Rubin J P, Marra K G. Adipose-derived stem cells: Implications in tissue regeneration. World J Stem Cells. 2014; 6 (3): 312-321).

In one example embodiment, AMSCs are administered in combination with bio-engineered materials (e.g., biomaterials, growth factors, plastic support, nanostructures, polymers, etc., as a support of a tissue or organ repair based on tissue engineering) (see, e.g., Mazini, et al., 2019). In another example embodiment, adipose tissue is generated in vivo using a combination of AMSCs and scaffolds. In an example embodiment, acellular scaffolds in combination with drugs or growth factors are used. Exemplary scaffolds, include, but are not limited to type I collagen, fibrin, silk fibroin, alginate, hyaluronic acid, and matrigel (see, e.g., Choi, et al., Adipose tissue engineering for soft tissue regeneration. Tissue Eng Part B Rev. 2010; 16:413 426; Tsuji, et al., Adipogenesis induced by human adipose tissue-derived stem cells. Tissue Eng Part A. 2009; 15:83 93; and Ito, et al., Adipogenesis using human adipose tissue-derived stromal cells combined with a collagen/gelatin sponge sustaining release of basic fibroblast growth factor. J Tissue Eng Regen Med. 2012: Epub ahead of print). In an example embodiment, injectable scaffolds are used, as minimally invasive therapies would be widely adapted by surgeons. In an example embodiment, methods of drug delivery include, but are not limited to using polymeric microspheres to control the release of factors such as bFGF, insulin, and dexamethasone (see, e.g., Marra, et al., FGF-2 enhances vascularization for adipose tissue engineering. Plast Reconstr Surg. 2008; 121:1153 1164; Kimura, et al., Time course of de novo adipogenesis in matrigel by gelatin microspheres incorporating basic fibroblast growth factor. Tissue Eng. 2002; 8:603-613; and Rubin, et al., Encapsulation of adipogenic factors to promote differentiation of adipose-derived stem cells. J Drug Target. 2009; 17:207-215). In one example embodiment, AMSCs are administered in a dose of about 1-5×10⁶ AMSCs/kg of body weight, however, the dose can be adjusted based on time and administration route and schedule.

### Allogenic Adipocyte Progenitor Donors

In one example embodiment, allogenic AMSCs are used for ACT. In another example embodiment, donors for allogenic AMSCs are screened for mutations/variants that decrease COBLL1 expression as described herein. In another example embodiment, COBLL1 expression is modified in allogenic cells even in situations where the cells do not have a COBLL1 variant or a decrease in function. In another example embodiment, increased COBLL1 expression or activity in transferred cells can compensate for host cells having decreased expression or activity. AMSCs are commonly known for their low immunogenicity and modulatory effects (see, e.g., Puissant, et al. Immunomodulatory effect of human adipose tissue-derived adult stem cells: comparison with bone marrow mesenchymal stem cells. Br J Haematol. 2005; 129 (1): 118-129). Less than 1% of AMSCs express the HLADR protein on their surface, leading to immunosuppressive effects and making them suitable for clinical applications in allogeneic transplantation and in therapies for the treatment of resistant immune disorders. Id. Further, adipogenic differentiated allogenic AMSCs can form new adipose tissue without immune rejection, such that adipogenic differentiated AMSCs can be used as a “universal donor” for soft-tissue engineering in MHC-mismatched recipients (see, e.g., Kim, et al., Clinical implication of allogenic implantation of adipogenic differentiated adipose-derived stem cells. Stem Cells Transl Med. 2014; 3 (11): 1312-1321).

In one example embodiment, the potential immunogenicity of allogeneic cells might cause their rejection after infusion. AMSC differentiation may alter their immunogenic phenotype, increasing HILA class-I and HLA class-II expression (see, e.g., Ceccarelli, et al., Immunomodulatory Effect of Adipose-Derived Stem Cells: The Cutting Edge of Clinical Application. Front Cell Dev Biol. 2020; 8:236). In another example embodiment, adipose tissue from HLA identical siblings, haplo-identical relatives, or HLA-screened healthy volunteers is used for collection and storage until used in an HLA-matched patient for allogenic transfer.

### Autologous Adipocyte Progenitor Donors

In one example embodiment, autologous AMSCs are used for ACT. In one embodiment, autologous AMSCs are used for chronic pathologies because the time required for the isolation and expansion of cells is not a limit given the non-acute nature of the diseases (e.g., T2D, lipodystrophy). In another example embodiment, autologous AMSCs are obtained from a subject in need thereof and cells for ACT are genetically modified using any of the methods described herein (e.g., repair of the mutation decreasing expression of COBLL1 or BCL2, overexpressing COBLL1 or BCL2 using gene therapy). CRISPR-Cas editing has been used to repair a variant in primary adipocytes and AMSCs (see, e.g., Claussnitzer, et al. FTO Obesity Variant Circuitry and Adipocyte Browning in Humans. N Engl J Med. 2015; 373 (10): 895-907).

### Pharmaceutical Formulations and Administration

Also described herein are pharmaceutical formulations that can contain an amount, effective amount, and/or least effective amount, and/or therapeutically effective amount of one or more compounds, molecules, compositions, vectors, vector systems, cells as described above, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a CRISPR-Cas system or component thereof described in greater detail elsewhere herein. In some embodiments, the pharmaceutical formulation can include, such as an active ingredient, a CRISPR-Cas polynucleotide described in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Type-2 Diabetes or a symptom thereof. In some embodiments, the subject in need thereof has or is suspected of having, a metabolic disease or disorder, insulin resistance, or glucose intolerance, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are promoting actin cytoskeleton remodeling processes, promoting accumulation of lipids in targeted cells, and promoting insulin-sensitivity.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/ml.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, pL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

### Viral Vector Formulation, Dosage, and Delivery

Compositions of the invention may be formulated for delivery to human subjects, as well as to animals for veterinary purposes (e.g. livestock (cattle, pigs, others)), and other non-human mammalian subjects. The dosage of the formulation can be measured or calculated as viral particles or as genome copies (“GC”)/viral genomes (“vg”). Any method known in the art can be used to determine the genome copy (GC) number of the viral compositions of the invention. In one example embodiment, the viral compositions can be formulated in dosage units to contain an amount of viral vectors that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of virus in the formulation is 1.0×10⁹ GC, 5.0×10⁹ GC, 1.0×10¹⁰ GC, 5.0×10¹⁰ GC, 1.0×10¹¹GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

The viral vectors can be formulated in a conventional manner using one or more physiologically acceptable carriers or excipients. The viral vectors may be formulated for parenteral administration by injection (e.g. by bolus injection or continuous infusion). Formulations for injection may be presented in unit dosage form (e.g. in ampoules or in multi-dose containers) with an added preservative. The viral compositions may take such forms as suspensions, solutions, or emulsions in oily or aqueous vehicles, and may contain formulatory agents such as suspending, stabilizing, or dispersing agents. Liquid preparations of the viral vector formulations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils), and preservatives (e.g. methyl or propyl-p-hydroxybenzoates or sorbic acid). The preparations may also contain buffer salts. Alternatively, the compositions may be in powder form for constitution with a suitable vehicle (e.g. sterile pyrogen-free water) before use.

### Recombinant Protein Formulation, Dosage, and Delivery

In one example embodiment, virus like particles (VLPs) are used to facilitate intracellular recombinant protein therapy (see, e.g., W (2020252455A1, U.S. Pat. No. 10,577,397B2). In certain embodiments, VLPs include a Gag-COBLL1 fusion protein. The Gag-COBLL1 fusion protein may include a matrix protein, a capsid protein, and/or a nucleocapsid protein covalently linked to COBLL1. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a COBLL1 fusion protein on the inside of the membrane, wherein COBLL1 is fused to a human-endogenous GAG or other plasma membrane recruitment domain (see, e.g., WO2020252455A1). Fusion proteins can be obtained using standard recombinant protein technology.

In one example embodiment, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17 (2): 263). In certain embodiments, cell-penetrating peptides can be conjugated to COBLL1, for example, using standard recombinant protein technology. In certain embodiments, cell-penetrating peptides can be concurrently delivered with recombinant COBLL1.

In one example embodiment, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9 (11): 3280-3292). Non-limiting nanocarriers include, but are not limited to nanoparticles (e.g., silica, gold), polymers, lipid based (e.g., cationic lipid within a polymer shell, lipid-like nanoparticles).

The pharmaceutical composition of the invention may be administered locally or systemically. In a preferred embodiment, the pharmaceutical composition is administered near the tissue whose cells are to be transduced. In a particular embodiment, the pharmaceutical composition of the invention is administered locally to the subcutaneous adipose tissue, which is composed of varying amounts of the two different types of adipose tissue: white adipose tissue (WAT) that stores energy in the form of triacylglycerol (TAG) and brown adipose tissue (BAT) that dissipates energy as heat, “burning” fatty acids to maintain body temperature. In one example embodiment, the pharmaceutical composition of the invention is administered in the white adipose tissue (WAT) and/or in the brown adipose tissue (BAT) by intra-WAT or intra-BAT injection. In another preferred embodiment, the pharmaceutical composition of the invention is administered systemically.

The “adeno-associated virus” (AAV) can be formulated with a physiologically acceptable carrier for use in gene transfer and gene therapy applications. The dosage of the formulation can be measured or calculated as viral particles or as genome copies (“GC”)/viral genomes (“vg”). Any method known in the art can be used to determine the genome copy (GC) number of the viral compositions of the invention. One method for performing AAV GC number titration is as follows: purified AAV vector samples are first treated with DNase to eliminate un-encapsulated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In any of the described methods the one or more vectors may be comprised in a delivery system. In any of the described methods the vectors may be delivered via liposomes, particles (e.g., nanoparticles), exosomes, microvesicles, a gene-gun. In any of the described methods viral vectors may be delivered by transduction of viral particles. The delivery systems may be administered systemically or by localized administration (e.g., direct injection). The term “systemically administered” and “systemic administration”, as used herein, means that the polynucleotides, vectors, polypeptides, or pharmaceutical compositions of the invention are administered to a subject in a non-localized manner. The systemic administration of the polynucleotides, vectors, polypeptides, or pharmaceutical compositions of the invention may reach several organs or tissues throughout the body of the subject or may reach specific organs or tissues of the subject. For example, the intravenous administration of a pharmaceutical composition of the invention may result in the transduction of more than one tissue or organ in a subject. The term “transduce” or “transduction”, as used herein, refers to the process whereby a foreign nucleotide sequence is introduced into a cell via a viral vector. The term “transfection”, as used herein, refers to the introduction of DNA into a recipient eukaryotic cell.

Recombinant protein compositions described herein may be administered systemically (e.g., intravenously) or administered locally to adipose tissue (e.g., injection). In preferred embodiments, the recombinant protein compositions are administered with an appropriate carrier to be administered to a mammal, especially a human, preferably a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions.

### CRISPR-Cas Delivery

The CRISPR-Cas systems disclosed herein may be delivered using vectors comprising polynucleotides encoding the Cas polypeptide and the guide molecule. For HDR based embodiments, the donor template may also be encoded on a vector. Vectors, dosages, and adipocyte-specific configurations suitable for delivery of these components include those discussed above.

The vector(s) can include regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector is to express an array of promoter-RNAs separated by cleavable sequences in the intron of a coding sequence or gene; and, in this instance, it is advantageous to use a polymerase II promoter, which can have increased expression and enable the transcription of long RNA in a tissue specific manner. (see, e.g., Chung K H, Hart C C, Al-Bassam S, et al. Polycistronic RNA polymerase II expression vectors for RNA interference based on BIC/miR-155. Nucleic Acids Res. 2006; 34 (7): e53). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters, especially as to the numbers of RNAs or guides discussed herein, without any undue experimentation.

The Cas polypeptide and guide molecule (and donor) may also be delivered as a pre-formed ribonucleoprotein complex (RNP). Delivery methods for delivery RNPs include virus like particles, cell-penetrating peptides, and nanocarriers discussed above.

Delivery mechanisms for CRISPRa systems include virus like particles, cell-penetrating peptides, and nanocarriers discussed above for CRISPR-Cas systems.

### Base Editing Delivery

Base editing systems may deliver on one or more vectors encoding the Cas-nucleobase deaminase and guide sequence. Vector systems suitable for this purpose includes those discussed above. Alternatively, base editing systems may be delivered as pre-complex Ribonucleoprotein complex (RNP. Systems for delving RNPs include the protein delivery systems: virus like particles; cell-penetrating peptides; and nanocarriers, discuss above.

A further example method for delivery of base-editing systems may include use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Diagnostic and Theranostic Methods

In another aspect, the variants resulting in reduced COBL11 expression may also be used in diagnostic and theranostic methods to detect increased risk for T2D and to guide treatment decisions.

In one example embodiment, a method for treating a subject suffering from, or at risk for, T2D comprises detecting one or more polygenic metabolic risk factors in a subject in need thereof, and administering one of the treatments for increasing COBL11 expression and/or COBL11 activity in adipocyte or adipocyte progenitors if the metabolic risk factors are detected, or administering a T2D standard of care if the metabolic risk factor is detected. In one example embodiment, the one or more risk indicators are selected from the group consisting of; a heterogenous lipid-associated morphological profile in visceral adipocytes, heterogeneity in lipid droplet size in visceral adipocytes, heterogeneity in lipid droplet number in visceral adipocytes, heterogeneity in lipid droplet distribution in visceral adipocytes, if the subject is post-menopausal, optionally older than 50 years old, increased adipocyte diameter, expression of one or more of the 51 genes in Table 6, up-regulation of one or more genes selected from the group consisting of ACAA1 and SCP2, expression of one or more genes selected from the group consisting of PLIN, ABHD5, MGLL, ATGL, and HIS as compared to an average level for adipocytes, increased lipid accumulation in matural visceral adipocytes, and reduced degradation in matural visceral adipocytes. In another example embodiment, the one or more risk factors are selected from the group consisting of higher intensity/ready of BODIPY, higher intensity/reading of mitochondrial-related intensity, higher count of BODIPY-related objects; and decreased BODIPY-related granularity, which may be detected using the methods described in the “Profiling Adipocyte Section” below.

In another example embodiment, a method for detecting T2D, or an increased risk of developing T2D, comprises detecting one or more variants associated with decreased expression of COBL11 or activity of COBL11, wherein detection of the one or more variants indicates a subject has, or is at an increase risk of developing T2D, or alternatively where the subject possesses a MONW/MOH risk phenotype. In certain example embodiments, the one or more variants include rs6712203. Detection of the one or more variants may be determined using any of the methods disclosed in the “Genotyping” section below. In certain example embodiments, the method may further comprise a treatment step comprising administering a therapeutically effective amount of one or more agents that a) increase the expression or activity of COBL11 or enhance actin remodeling in adipocyte or adipocyte-progenitors, b) a gene editing system the corrects one or more variants to a wild-type or non-risk variant, or c) adoptive cell transfer comprising allogenic or autologous adipocyte donors as disclosed in the therapeutic embodiments above.

In another example embodiment, a method for detecting lipodystrophy, or an increased risk of developing lipodystrophy, comprises detecting one or more variants associated with decreased expression of BCL2 and/or KDSR or activity of BCL2 and/or KDSR, or detecting one or more variants associated with increased expression of VPS4B or activity of VPS4B wherein detection of the one or more variants indicates a subject has, or is at an increased risk of developing lipodystrophy, or alternatively where the subject possesses a lipodystrophy risk phenotype. In certain example embodiments, the one or more variants include rs12454712. Detection of the one or more variants may be determined using any of the methods disclosed in the “Genotyping” section below. In certain example embodiments, the method may further comprise a treatment step comprising administering a therapeutically effective amount of one or more agents that a) increase the expression or activity of BCL2 and/or KDSR or decrease expression of VPS4B, b) a gene editing system the corrects one or more variants to a wild-type or non-risk variant, or c) adoptive cell transfer comprising allogenic or autologous adipocyte donors as disclosed in the therapeutic embodiments above.

In another example embodiments, a method of treating T2D comprises performing a genotyping assay on a biological sample from a subject to determine if the subject has one or more risk variants that decrease COBL11 expression or activity, and administering one of the therapeutic modalities described above in the “Methods of Treatment” section if the one or more variants are detected, or administering a T2D standard-of-care therapy, as further defined below, if the one or more variants are not detected. In one example embodiment, the one or more variants comprise rs6712203.

In an example embodiment, a method of treating lipodystrophy comprises performing a genotyping assay on a biological sample from a subject to determine if the subject has one or more risk variants that decrease BCL2 and/or KDSR expression or activity, or one or more risk variants that increase VPS4B expression or activity, and administering one of the therapeutic modalities described above in the “Methods of Treatment” section if the one or more variants are detected, or administering a T2D standard-of-care therapy, as further defined below, if the one or more variants are not detected. In one example embodiment, the one or more variants comprise rs12454712.

### Genotyping Assays

In any of the above diagnostic/theranostic embodiments, identifying whether a metabolic risk factor is present includes obtaining information regarding the identity (i.e., of a specific nucleotide), presence or absence of one or more specific risk loci in a subject. Determining the presence of a risk loci can, but need not, include obtaining a sample comprising DNA from a subject. The individual or organization who determines the presence of an risk loci need not actually carry out the physical analysis of a sample from a subject; the methods can include using information obtained by analysis of the sample by a third party. Thus, the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility. Identifying the presence of a risk loci can be done by any DNA detection method known in the art, including sequencing at least part of a genome of one or more cells from the subject. In certain example embodiments, risk loci are detected via detection of a single nucleotide polymorphism (SNP), e.g., rs6712203.

SNPs may be detected through hybridization-based methods, including dynamic allele-specific hybridization (DASH), molecular beacons, and SNP microarrays, enzyme-based methods including RFLP, PCR-based, e.g., allelic-specific polymerase chain reaction (AS-PCR), polymerase chain reaction restriction fragment length polymorphism (PCR-RFLP), multiplex PCR real-time invader assay (mPCR-RETINA), (amplification refractory mutation system (ARMS), Flap endonuclease, primer extension, 5′ nuclease, e.g., Taqman or 5′nuclease allelic discrimination assay, and oligonucleotide ligation assay, and methods such as single strand conformation polymorphism, temperature gradient gel electrophoresis, denaturing high performance liquid chromatography, high-resolution melting of the entire amplicon, use of DNA mismatch-binding proteins, SNPlex, and Surveyor nuclease assay.

In certain example embodiments, detection of SNPs can be done by sequencing. Sequencing can be, for example, whole genome sequencing. In one example embodiment, the invention involves high-throughput and/or targeted nucleic acid profiling (for example, sequencing, quantitative reverse transcription polymerase chain reaction, and the like).

In certain embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56 (2): 61-77; Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi: 10.1002/0471142727.mb0422s107 (2014). PMCID: 4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437:376-80); Schneider and Dekker (Nat Biotechnol. 2012 Apr. 10; 30 (4): 326-8); Ronaghi et al (Analytical Biochemistry 1996 242:84-9); Shendure et al (Science 2005 309:1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

In certain embodiments, the present invention includes whole genome sequencing. Whole genome sequencing (also known as WGS, full genome sequencing, complete genome sequencing, or entire genome sequencing) is the process of determining the complete DNA sequence of an organism's genome at a single time. This entails sequencing all of an organism's chromosomal DNA as well as DNA contained in the mitochondria and, for plants, in the chloroplast. “Whole genome amplification” (“WGA”) refers to any amplification method that aims to produce an amplification product that is representative of the genome from which it was amplified. Non-limiting WGA methods include Primer extension PCR (PEP) and improved PEP (I-PEP), Degenerated oligonucleotide primed PCR (DOP-PCR), Ligation-mediated PCR (LMP), T7-based linear amplification of DNA (TLAD), and Multiple displacement amplification (MDA).

In certain embodiments, the present invention includes whole exome sequencing. Exome sequencing, also known as whole exome sequencing (WES), is a genomic technique for sequencing all of the protein-coding genes in a genome (known as the exome) (see, e.g., Ng et al., 2009, Nature volume 461, pages 272-276). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology. In certain embodiments, whole exome sequencing is used to determine mutations in genes associated with disease.

In certain embodiments, targeted sequencing is used in the present invention (see, e.g., Mantere et al., PLOS Genet 12 e1005816 2016; and Carneiro et al. BMC Genomics, 2012 13:375). Targeted gene sequencing panels are useful tools for analyzing specific mutations in a given sample. Focused panels contain a select set of genes or gene regions that have known or suspected associations with the disease or phenotype under study. In certain embodiments, targeted sequencing is used to detect mutations associated with a disease in a subject in need thereof. Targeted sequencing can increase the cost-effectiveness of variant discovery and detection.

### Standard of Care Therapies

As noted above, when a metabolic risk factor is not detected, a standard of care therapy may be administered instead. A standard of care therapy may comprise administration metformin, thiazolidinediones (glitazones), biguanides, meglitinides, DPP-4 inhibitors, Sodium-glucose transporter 2 (SGLT2) inhibitors, alpha-glucosidase inhibitors, bile acid sequestrants, incretin based therapies, sulfonylureas and amylin analogs. In some embodiments, the biguanide is a metformin. In some embodiments, the meglitinide is repaglinide or nateglinide. Sulfonylureas include, for example, chlorpropamide, glipizide, glyburide and glimepiride. Rosiglitazone (Avandia) and pioglitazone (ACTOS) are exemplary thiazolidinediones. DPP-4 inhibitors include Sitagliptin (Januvia), saxagliptin (Onglyza), linagliptin (Tradjenta), alogliptin (Nesina). Sodium-glucose transporter 2 (SGLT2) inhibitors include Canagliflozin (Invokana) and dapagliflozin (Farxiga). Acarbose (Precose) and miglitol (Glyset) are exemplary alpha-glucosidase inhibitors. An exemplary bile acid sequestrate is colesevelam (Welchol) which is a cholesterol-lowering medication that can reduce blood glucose levels. In some embodiments, more than one drug can be used in a combination therapy, in particular when the drugs act in different ways to lower blood glucose levels. Treatment may also include, alone, or in addition to drug therapy, intensive lifestyle interventions including modifications to diet and exercise. Initiating a treatment can include devising a treatment plan based on the risk group, which corresponds to the PRS calculated for the subject. In some embodiments, the polygenic risk score is used to guide enhanced monitoring strategies. In some embodiments, the polygenic risk score is used to guide intensive lifestyle interventions. As used herein, “polygenic risk score” refers to an assessment of the risk of a specific condition based on the collective influence of many genetic variants or a score based on the number of variants related to the disease a subject has.

In certain example embodiments, where a metabolic risk factor is detected, the methods of treatment for increasing COBL11, BCL2 or KDSR expression or COBL11, BCL2 or KDSR activity disclosed herein may also be co-administered with a standard of care therapy. Similarly, in an example embodiment, where a metabolic risk factor is detected, the methods of treatment for decreasing VPS4B expression or VPS4B activity disclosed herein may also be co-administered with a standard of care therapy.

### Additional Targets for Treating and Diagnosing Metabolic Diseases

Applicants have performed functional analysis (morphological and histological) of additional SNPs associated with metabolic diseases. For example, SNPs in the BCL2 gene result in cellular phenotypes associated with lipodystrophy. Lipodystrophy syndromes are a group of genetic or acquired disorders in which the body is unable to produce and maintain healthy fat tissue. Other SNPs analyzed using the methods of the present invention include rs9686661, rs4804833, rs2972144, rs13389219, rs11837287, TCF71.2, rs1534696 (SNX10), rs287621, rs1412956, rs13133548, rs11667352, rs12454712 (BCL2), rs673918, rs646123, rs2963449, rs1572993, rs632057, rs11637681, rs6063048, rs7660000, rs1421085, rs7258937, rs9939609, rs998584, rs4925109, and rs12641088. In certain embodiments, the present invention provides for a method of treating subjects suffering from or at risk of developing a metabolic disease, comprising administering a gene editing system that corrects one or more genomic risk variants selected from the group consisting of rs9686661, rs4804833, rs2972144, rs13389219, rs11837287, TCF71.2, rs1534696 (SNX10), rs287621, rs1412956, rs13133548, rs11667352, rs12454712 (BCL2), rs673918, rs646123, rs2963449, rs1572993, rs632057, rs11637681, rs6063048, rs7660000, rs1421085, rs7258937, rs9939609, rs998584, rs4925109, and rs12641088. In certain embodiments, the present invention provides for a method of diagnosing subjects suffering from or at risk of developing a metabolic disease, comprising detecting one or more genomic risk variants selected from the group consisting of rs9686661, rs4804833, rs2972144, rs13389219, rs11837287, TCF7L2, rs1534696 (SNX10), rs287621, rs1412956, rs13133548, rs11667352, rs12454712 (BCL2), rs673918, rs646123, rs2963449, rs1572993, rs632057, rs11637681, rs6063048, rs7660000, rs1421085, rs7258937, rs9939609, rs998584, rs4925109, and rs12641088.

### Profiling Adipocyte Tissue

### LipocyteProfiler

In certain embodiments, high-throughput multiplex profiling for simultaneously identifying morphological and cellular phenotypes is performed on cellular system. The cellular system may be a homogenous population of cells. The cellular system may be derived from a subject. The subject can be a control healthy subject or a subject having a specific clinical phenotype. Methods of obtaining cells from a subject are known in the art and are described further herein. The cellular system can include cells that were isolated and expanded or differentiated. In preferred embodiments, the cellular system may comprise lipid-accumulating cells. The lipid accumulating cells may be lipocytes. As used herein, lipocytes are any fat storing cell. The lipocytes may be adipocytes, hepatocytes, macrophages/foam cells and glial cells. The lipocytes may be part of a pathophysiological process in cells that include fat storing cells, such as, vascular smooth muscle cells, skeletal muscle cells, renal podocytes, and cancer cells. In certain embodiments, high-throughput multiplex and simultaneous profiling of morphological and cellular phenotypes is performed on adipose tissue or adipose cells (e.g., AMSCs, adipocytes). As used herein, adipocytes, also known as lipocytes and fat cells, are the cells that primarily compose adipose tissue, specialized in storing energy as fat. Adipocytes are derived from mesenchymal stem cells which give rise to adipocytes through adipogenesis. The cellular system may include stem cells differentiated over a time course, wherein the cells from the cellular system are stained and imaged at different time points. The time points may be one or more days of differentiation, such as, but not limited to 0 days, 1 day, 2 days, 3 days, 4 days, 5 days, 6 days, 7 days, 8 days, 9 days, 10 days, 11 days, 12 days, 13 days or 14 or more days. The stem cells may be mesenchymal stem cells (AMSCs) differentiated to adipocytes. The AMSCs may be obtained from a subject. The AMSCs may be subcutaneous AMSCs. The AMSCs may be visceral AMSCs. The adipose tissue beneath the skin is called subcutaneous adipose tissue (SAT), whereas the one lining internal organs is termed visceral adipose tissue (VAT).

The method can include a combination of fluorescent dyes that are used to stain various biological models present in adipocytes. The cells can be imaged simultaneously. The images can be analyzed by an automated image analysis pipeline to identify morphological and cellular phenotypes from the resulting images.

In certain embodiments, the cellular system is stained to differentiate cellular compartments. The cellular compartments can include the nucleus, cytoplasm or the entire cell (e.g., including nucleus and cytoplasm). In certain embodiments, the cellular system is stained to differentiate organelles. The organelles can include DNA (e.g., genomic DNA), mitochondria, actin, golgi, plasma membrane, lipids (e.g. lipid containing vesicles), nucleoli and cytoplasmic RNA. In certain embodiments, actin, golgi, plasma membrane are represented as a single organelle (AGP). In certain embodiments, the stain can indicate intensity, granularity, and/or texture for each stained compartment or organelle. The size and shape of each identified object can be determined (e.g., lipid droplets). The colocalization, number of objects, and distance to neighboring objects can also be determined by staining. Methods of staining non-lipocyte cells may be used, such as, CellPainting (Bray M A, Singh S, Han H, et al. Cell Painting, a high-content image-based assay for morphological profiling using multiplexed fluorescent dyes. Nat Protoc. 2016; 11 (9): 1757-1774).

In certain embodiments, features can be extracted from the images. In certain embodiments, the features are categorized based on a range of values for each feature. For example each separate feature can be divided into at least 2 categories based on dividing the values based on a range. Each separate features may be divided into 2, 3, 4, 5, 6, 7, 8, 9, or 10 or more sub features. For example, object size may be divided into 5 size categories. Each size category may have different categories of intensity, texture or granularity. Features can be combinations of object size, object shape, intensity, granularity, texture, colocalization, number of objects, distance to neighboring objects, and/or cellular compartment (see tables and figures for example features).

A number of bioimaging software packages (free and commercial) exist for morphological feature extraction (Eliceiri K W, et al. Biological imaging software tools. Nat Methods. 2012; 9:697 710). In one example, CellProfiler and a novel pipeline can be used to automate imaging (see, e.g., Carpenter et al., (2006) CellProfiler: image analysis software for identifying and quantifying cell phenotypes. Genome Biology 7: R100. PMID: 17076895; and Kamentsky et al., (2011) Improved structure, function, and compatibility for CellProfiler: modular high-throughput image analysis software. Bioinformatics 2011/doi. PMID: 21349861 PMCID: PMC3072555). The image feature extraction workflow for Cell Painting is divided into three tasks, each of which is performed by a CellProfiler pipeline: (a) illumination correction, (b) quality control, and (c) morphological feature extraction.

In one example embodiment the features can be linked to specific phenotypes. The phenotypes can be specific gene programs (biological programs) by comparing features to gene programs in the same cellular system and by determining genes associated with morphological characteristics. As used herein the term “gene program” or “biological program” can be used interchangeably with “expression program” and refers to a set of biomarkers that share a role in a biological function (e.g., lipolysis). Biological programs can include a pattern of biomarker expression that result in a corresponding physiological event or phenotypic trait. Biological programs can include up to several hundred biomarkers that are expressed in a spatially and temporally controlled fashion. The phenotypes can be specific clinical features. In certain embodiments, features associated with clinical characteristics are identified by comparing features in a control group of subjects having a clinical characteristic. Clinical characteristics can include risk for a disease, such as type 2 diabetes (T2D), coronary disease. Clinical characteristics can also include, age, weight, BMI, etc.

In certain embodiments, more than one cell needs to be imaged in order to determine morphological features for a subject or cellular system. In example embodiments, 50 or more cells per cellular system are imaged, more preferably, more than 100, more preferably about 500 or more cells are imaged per cellular system.

In certain embodiments, a cellular system is stained with one or more fluorescent dyes. As used herein, the terms “fluorescent dye”, “reactive dye”, or “fluorophore” are used herein interchangeably. They refer to non-protein molecules that absorb photons and re-emit them. Fluorescent dyes typically contain several combined aromatic groups, or planar or cyclic molecules with several x-bonds. Fluorescent dyes are usually targeted to proteins of interest by antibody conjugates or peptide tags. Fluorescent dyes may be used alone, as a tracer fluid, as a dye for staining of certain structures, or as a probe or indicator. As an indicator, a fluorescent dye may fluoresce as a result of its environment, such as but not limited to, polarity or ions.

In one example embodiment, one or more fluorescent dyes are selected from the group consisting of Hoechst, Phalloidin, WGA, MitoTracker Red, BODIPY, and SYTO14. As used herein, “Hoechst” and “Hoechst 33342” are used interchangeably. The CAS name for Hoechst is 2,5′-1II-benzimidazole, 2′-(4-ethoxyphenyl)-5-(4-methyl-1-piperazinyl). Hoechst is a bis-benzimide derivative that binds to AT-rich sequences in the minor groove of double-stranded DNA. The emission wavelengths of Hoechst are in the red visible spectrum around 630-650 nm and the blue visible spectrum around 405-450 nm.

Phalloidin is a bicyclic peptide that belongs to a class of toxins called phallotoxins that binds to F-actin. These phallotoxins are isolated from Amanita phalloides. Phalloidin conjugates include: Alexa Fluor 350 Phalloidin, whose excitation/emission wavelength is around 346/442 nm respectively; NBD phallacidin, whose excitation/emission wavelength is around 465/536 nm respectively; Alexa Fluor Plus 405 Phalloidin, whose excitation/emission wavelength is around 405/450 nm respectively; Fluorescein phalloidin, whose excitation/emission wavelength is around 496/516 nm respectively; Alexa Fluor 488 Phalloidin, whose excitation/emission wavelength is around 496/519 nm respectively; Oregon Green 488 phalloidin, whose excitation/emission wavelength is around 496/520 nm respectively; Rhodamine phalloidin, whose excitation/emission wavelength is around 540/565 nm respectively; Alexa Fluor Plus 555 phalloidin, whose excitation/emission wavelength is around 555/565 nm respectively; BODIPY 558/568 phalloidin, whose excitation/emission wavelength is around 558/569 nm respectively; Alexa Fluor 594 Phalloidin, whose excitation/emission wavelength is around 590/617 nm respectively; Texas Red-X phalloidin, whose excitation/emission wavelength is around 591/608 nm respectively; Alexa Fluor Plus 647 phalloidin, whose excitation/emission wavelength is around 650/668 nm respectively; Alexa Fluor 680 Phalloidin, whose excitation/emission wavelength is around 679/702 nm respectively; Biotin-XX Phalloidin; and Alexa Fluor Plus 750 Phalloidin, whose excitation/emission wavelength is around 758/784 nm respectively.

Wheat germ agglutinin or WGA is a carbohydrate-binding protein. The excitation/emission wavelengths are around 495/519 nm respectively.

MitoTracker Deep Red is a highly conjugated compound that selectively binds to mitochondria. Additional MitoTracker probes comprise of: MitoTracker Green FM, whose absorption/emission wavelength is around 490/516 nm respectively; MitoTracker Orange CMTMRos, whose absorption/emission wavelength is around 551/576 nm respectively; MitoTracker Orange CM-H2TMRos, whose absorption/emission wavelength is around 551/576 nm respectively; MitoTracker Red CMXRos, whose absorption/emission wavelength is around 578/599 nm respectively; MitoTracker Red CM-H2XRos, whose absorption/emission wavelength is around 578/599 nm respectively; MitoTracker Red FM, whose absorption/emission wavelength is around 581/644 nm respectively

As used herein, the terms “BODIPY”, “dipyrromethencboron difluoride”, and “boron-dipyrromethene” are used herein interchangeably. The BODIPY IUPAC name is 4,4-difluoro-4-bora-3a,4a-diaza-s-indacene. BODIPY probes have fluorescence excitation maxima from around 500-600 nm and emission maxima from around 510-665 nm. In one example embodiment, BODIPY refers to BODIPY 505/515, whose excitation/emission wavelength is around 502/512 nm respectively. In another example embodiment, BODIPY probes comprise of: BODIPY FL, whose absorption/emission wavelength is around 503/512 nm respectively; BODIPY R6G, whose absorption/emission wavelength is around 528/547 nm respectively; BODIPY TMR, whose absorption/emission wavelength is around 544/570 nm respectively; BODIPY 581/591, whose absorption/emission wavelength is around 581/591 nm respectively; BODIPY TR, whose absorption/emission wavelength is around 588/616 nm respectively; BODIPY 630/650, whose absorption/emission wavelength is around 625/640 nm respectively; BODIPY 650/665, whose absorption/emission wavelength is around 646/660 nm respectively.

SYTO14 dye binds to both DNA and RNA. STYO14 probes have fluorescence excitation/emission wavelength is around 517/549 nm for DNA and 521/547 for RNA respectively. Addition SYTO dyes include: SYTO 40 blue-fluorescent nucleic acid stain, whose excitation/emission wavelength is around 419/445 nm respectively; SYTO 41 blue-fluorescent nucleic acid stain, whose excitation/emission wavelength is around 426/455 nm respectively; SYTO 42 blue-fluorescent nucleic acid stain, whose excitation/emission wavelength is around 430/460 nm respectively; SYTO 45 blue-fluorescent nucleic acid stain, whose excitation/emission wavelength is around 452/484 nm respectively; SYTO RNASelect green-fluorescent cell stain, whose excitation/emission wavelength is around 490/530 nm respectively; SYTO 9 green-fluorescent nucleic acid stain, whose excitation/emission wavelength is around 483/503 nm respectively; SYTO 10 green-fluorescent nucleic acid stain, whose excitation/emission wavelength is around 484/505 nm respectively; SYTO BC green-fluorescent nucleic acid stain, whose excitation/emission wavelength is around 485/500 nm respectively; SYTO 13 green-fluorescent nucleic acid stain, whose excitation/emission wavelength is around 488/509 nm respectively; SYTO 16 green-fluorescent nucleic acid stain, whose excitation/emission wavelength is around 488/518 nm respectively; SYTO 24 green-fluorescent nucleic acid stain, whose excitation/emission wavelength is around 490/515 nm respectively; SYTO 21 green-fluorescent nucleic acid stain, whose excitation/emission wavelength is around 494/517 nm respectively; SYTO 12 green-fluorescent nucleic acid stain, whose excitation/emission wavelength is around 500/522 nm respectively; SYTO 11 green-fluorescent nucleic acid stain, whose excitation/emission wavelength is around 508/527 nm respectively; SYTO 25 green-fluorescent nucleic acid stain, whose excitation/emission wavelength is around 521/556 nm respectively; SYTO 81 orange-fluorescent nucleic acid stain, whose excitation/emission wavelength is around 530/544 nm respectively; SYTO 80 orange-fluorescent nucleic acid stain, whose excitation/emission wavelength is around 531/545 nm respectively; SYTO 82 orange-fluorescent nucleic acid stain, whose excitation/emission wavelength is around 541/560 nm respectively; SYTO 83 orange-fluorescent nucleic acid stain, whose excitation/emission wavelength is around 543/559 nm respectively; SYTO 84 orange-fluorescent nucleic acid stain, whose excitation/emission wavelength is around 567/582 nm respectively; SYTO 85 orange-fluorescent nucleic acid stain, whose excitation/emission wavelength is around 567/583 nm respectively; SYTO 64 red-fluorescent nucleic acid stain, whose excitation/emission wavelength is around 598/620 nm respectively; SYTO 61 red-fluorescent nucleic acid stain, whose excitation/emission wavelength is around 620/647 nm respectively; SYTO 17 red-fluorescent nucleic acid stain, whose excitation/emission wavelength is around 621/634 nm respectively; SYTO 59 red-fluorescent nucleic acid stain, whose excitation/emission wavelength is around 622/645 nm respectively; SYTO 62 red-fluorescent nucleic acid stain, whose excitation/emission wavelength is around 649/680 nm respectively; SYTO 60 red-fluorescent nucleic acid stain, whose excitation/emission wavelength is around 652/678 nm respectively; and SYTO 63 red-fluorescent nucleic acid stain, whose excitation/emission wavelength is around 654/675 nm respectively;

In certain embodiment, a dye may be a non-protein organic dye belonging to a family such as Xanthene, Cyanine, Squaraine, Squaraine rotaxane, Naphthalene, Coumarin, Oxadiazole, Anthracene, Pyrene, Oxazine, Acridine, Arylmethine, Tetrapyrrole, Dipyrromethenc.

In certain embodiment, a dye may be a fluorescent protein such as green fluorescent protein (GFP), enhanced green fluorescent protein (EGFP), red fluorescent protein (RFP), blue fluorescent protein (BFP), cyan fluorescent protein (CFP), yellow fluorescent protein (YFP), miRFP, miRFP670, mCherry, tdTomato, DsRed-Monomer, DsRed-Express, DSRed-Express2, DsRed2, AsRed2, mStrawberry, mPlum, mRaspberry, HcRed1, E2-Crimson, mOrange, mOrange2, mBanana, ZsYellow1, TagBFP, mTagBFP2, Azurite, EBFP2, mKalamal, Sirius, Sapphire, T-Sapphire, ECFP, Cerulean, SCFP3A, mTurquoise, m′Turquoise2, monomelic Midoriishi-Cyan, TagCFP, niTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mK02, mTangerine, mApple, mRuby, mRuby2, HcRed-Tandem, mKate2, mNeptune, NiFP, mkeima Red, LSS-mKatel, LSS-mKate2, mBeRFP, PA-GFP, PAmCherry1, PATagRFP, TagRFP6457, IFP1.2, iRFP, Kaede (green), Kaede (red), KikGR1 (green), KikGR1 (red), PS-CFP2, mLos2 (green), mEos2 (red), mEos3.2 (green), mEos3.2 (red), PSmOrange, Dronpa, Dendra2, Timer, AmCyan1, GFPuv, mCFP, CyPct, mKeima-Red, AmCyan1, mTFP1, Midoriishi Cyan, Wild Type GFP, TurboGFP, ZsGreen1, FYFP, Topaz, mCitrine, YPet, Turbo YFP, ZsYellow1, Kusabira Orange, Allophycocyanin, TurboRFP, DsRed monomer, TurboFP602, mRFP1, J-Red, R-phycocrythrin, RPE, B-phycoerythrin, BPE, HcRed1, Katusha, Peridinin Chlorophyll, PerCP, TagFP635, TurboFP635, or a combination thereof.

In certain embodiment, a dye may be a cell function dye such as Indo-1, Fluo-3, Fluo-4, DCFH, DHR, SNARF.

In certain embodiment, a dye may be a nucleic acid dye such as DAPI, SYTOX Blue, Chromomycin A3, Mithramycin, YOYO-1, Ethidium Bromide, Acridine Orange, SYTOX Green, TOTO-1, TO-PRO-1, TO-PRO: Cyanine Monomer, Thiazole Orange, CyTRAK Orange, Propidium Iodide (PI), LDS 751, 7-AAD, SYTOX Orange, TOTO-3, TO-PRO-3, DRAQ5, DRAQ7

In certain embodiment, a dye may be a Reactive and conjugated dye such as Allophycocyanin (APC), Aminocoumarin, APC-Cy7 conjugates, Cascade Blue, Cy2, Cy3, Cy3.5, Cy3B, Cy5, Cy5.5, Cy7, Fluorescein, FluorX, G-Dye100, G-Dye200, G-Dye300, G-Dye400, Hydroxycoumarin, Lissamine Rhodamine B, Lucifer yellow, Methoxycoumarin, NBD, Pacific Blue, Pacific Orange, PE-Cy5 conjugates, PE-Cy7 conjugates, PerCP, R-Phycoerythrin (PE), Red 613, Texas Red, TRITC, TruRed, X-Rhodaminc.

In certain embodiment, a dye may be CF dye, DRAQ and CyTRAK probes, EverFluor, Alexa Fluor, Bella Fluor, Dylight Fluor, Atto and Tracy, FluoProbes, Abberior Dyes, DY and MegaStokes Dyes, Sulfo Cy dyes, HiLyte Fluor, Seta, SeTau and Square Dyes, Quasar and Cal Fluor dyes, SureLight Dyes, APC, APCXL, RPE, BPE, Vio Dyes.

In certain embodiments, morphological profiling is performed on a cellular system and RNA-seq is performed on the same cellular system. In certain embodiments, a separate sample of the cellular system is sequenced. Thus, in one example, RNA-seq data can be linked to morphological imaging data. In certain embodiments, a transcriptome is sequenced. As used herein the term “transcriptome” refers to the set of transcripts molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, samples or cells are clustered based on the features identified. Clustering can use features from varying sources (e.g., LipocyteProfiler, RNA-seq) (see, e.g., International Application No. PCT/US2018/061348).

In certain embodiments, morphological features and optionally gene programs are determined for a SNP of interest. For example, cells are stained that include a SNP and where the SNP is active (e.g., a gene is expressed that is under control of a regulatory element comprising the SNP) or expressed (i.e., the SNP is expressed in the cell type). The function of the SNP may be determined based on determining morphological features. In certain embodiments, morphological features and optionally gene programs are determined for a candidate drug. In certain embodiments, the drug is suspected to alter one or more characteristics of a lipid accumulating cell. In certain embodiments, features associated with perturbation of one or more genomic loci are determined. In preferred embodiments, a cellular system is perturbed with a programmable nuclease system as described herein or an RNAi system as described herein.

In certain embodiments, clinical characteristics can be predicted by determining features for a cellular system obtained from a subject and comparing the features to features identified for a characteristic. In certain embodiments, the features are chosen by fitting a logistic regression model for the clinical characteristic on the entire set of features identified for subjects having a characteristic. Features can be further determined by connecting features in a network and generating a cutoff value to select features with a specific weight of interaction with other features. In another embodiment, features can be the number of features that can be modeled in a specific compartment category. The features that can be modeled can be adjusted based on cutoff values for each feature.

The logistic regression model may be a linear model with logit link (GLM). The linear association with binomial distribution may be implemented using the R glm function. The default glm convergence criteria on deviances may be used to stop the iterations. The DeLong method may be used to calculate confidence intervals for the c-statistics. Forward feature selection (R step function) may be used to select the features. The Akaike information criterion (AIC) may be used as the stop condition for the feature selection procedure. Histology

In certain embodiments, histological staining is performed on a tissue sample. The tissue sample may be obtained from a subject. The subject can be a control healthy subject or a subject having a specific clinical phenotype. Methods of obtaining tissues from a subject are known in the art and are described further herein. In certain embodiments, the tissue sample comprises lipid-accumulating cells. In preferred embodiments, the tissue sample is adipose tissue. The adipose tissue may be subcutaneous adipose tissue (SAT) or visceral adipose tissue (VAT).

Histology, also known as microscopic anatomy or microanatomy, is the branch of biology which studies the microscopic anatomy of biological tissues. Histology is the microscopic counterpart to gross anatomy, which looks at larger structures visible without a microscope. Although one may divide microscopic anatomy into organology, the study of organs, histology, the study of tissues, and cytology, the study of cells, modern usage places these topics under the field of histology. In medicine, histopathology is the branch of histology that includes the microscopic identification and study of diseased tissue. Biological tissue has little inherent contrast in either the light or electron microscope. Staining is employed to give both contrast to the tissue as well as highlighting particular features of interest. When the stain is used to target a specific chemical component of the tissue (and not the general structure), the term histochemistry is used. Antibodies can be used to specifically visualize proteins, carbohydrates, and lipids. This process is called immunohistochemistry, or when the stain is a fluorescent molecule, immunofluorescence. This technique has greatly increased the ability to identify categories of cells under a microscope. Other advanced techniques, such as nonradioactive in situ hybridization, can be combined with immunochemistry to identify specific DNA or RNA molecules with fluorescent probes or tags that can be used for immunofluorescence and enzyme-linked fluorescence amplification.

In certain embodiments, features are extracted from the histological images (see, e.g., Glastonbury C A, Pulit S L, Honecker J, et al. Machine Learning based histology phenotyping to investigate the epidemiologic and genetic basis of adipocyte morphology and cardiometabolic traits. PLOS Comput Biol. 2020; 16 (8): c1008044. Published 2020 Aug. 14. doi: 10.1371/journal.pcbi.1008044). Applicants have identified specific cell area features that associate with clinical features. Previously, cell area could only be associated to BMI (Glastonbury, et al. 2020). In certain embodiments, the histological features are cell area (μm²) features. In certain embodiments, the histological features are cell shape features. In one exemplary embodiment, cell area features include 5, 6, 7, 8, 9, 10, 15, or 20 or more features, preferably 20 features. The features may be determined by grouping cells into two or more size categories (e.g., 5). The size categories may be “very small”, “small”, “medium”, “large” and “very large.” The size categories may be determined by determining cell areas for the same tissue type in a large cohort of the same tissue type (e.g., control group). The cohort may include healthy and diseased subjects. In an example embodiment, the categories are determined by grouping cells according to: cell area <25% quartile point for the control group (very small), cell area ≥25% quartile point for the control group and <the median cell area for the control group (small), cell area ≥median cell area for the control group and <mean cell area for the control group (medium), cell area ≥mean area for the control group and <75% quartile point for the control group (large), and cell area ≥75% quartile point for the control group (very large). The size categories above would, for example, result in 5 features. Each size category can be further divided to determine further features. For example, each size category can be divided into 2, 3, 4 or more features. In an example embodiment, each size category is divided based on the fraction of cells in the cell area category, median area of cells in the category, 25% interquartile point in the category, and 75% interquartile point in the category. Thus, the features in this example that can be determined for each tissue sample would be 20 features. In an example embodiment, the 20 features can be used to predict clinical features that could not be predicted with previous cell area methods. Moreover, the features can be used to predict morphological features. Combining predictions made using both histological and morphological features may provide an improved prediction.

In one example embodiment the features can be linked to specific phenotypes. The phenotypes can be specific gene programs (biological programs) by comparing features to gene programs in the tissue sample and by determining genes associated with histological characteristics. The phenotypes can be specific clinical features. In certain embodiments, features associated with clinical characteristics are identified by comparing features in a control group of subjects having a clinical characteristic. Clinical characteristics can include risk for a disease, such as type 2 diabetes (T2D), coronary disease. Clinical characteristics can also include, age, weight, BMI, etc.

In certain embodiments, more than one cell needs to be imaged in order to determine histological features for a subject. In example embodiments, 50 or more cells per tissue sample are imaged, more preferably, more than 100, more preferably about 500 or more cells are imaged per tissue sample.

In certain embodiments, histological features and optionally gene programs are determined for a SNP of interest. For example, tissues are stained from a subject having a SNP and where the SNP is active (e.g., a gene is expressed that is under control of a regulatory element comprising the SNP) or expressed in the tissue. The function of the SNP may be determined based on determining histological features. In certain embodiments, histological features and optionally gene programs are determined for a candidate drug. In certain embodiments, the drug is suspected to alter one or more characteristics of a lipid accumulating cell. For example, a subject or animal model is treated with a drug before histological analysis, In certain embodiments, features associated with perturbation of one or more genomic loci are determined. In preferred embodiments, a cellular system is perturbed in vivo (e.g., animal model) with a programmable nuclease system as described herein or an RNAi system as described herein.

In certain embodiments, clinical characteristics can be predicted by determining histological features for a tissue obtained from a subject and comparing the features to features identified for a characteristic. In certain embodiments, the features are chosen by fitting a logistic regression model for the clinical characteristic on the entire set of features identified for subjects having a characteristic. The logistic regression model may be a linear model with logit link (GLM). The linear association with binomial distribution may be implemented using the R glm function. The default glm convergence criteria on deviances may be used to stop the iterations. The DeLong method may be used to calculate confidence intervals for the c-statistics. Forward feature selection (R step function) may be used to select the features. The Akaike information criterion (AIC) may be used as the stop condition for the feature selection procedure.

### Screening Methods

### Identifying Novel and Improved Treatments

In certain embodiments, the cell subset frequency and/or differential cell states (e.g., intrinsic immune response) can be detected for screening of novel therapeutic agents. In certain embodiments, the present invention can be used to identify improved treatments by monitoring the identified cell states in a subject undergoing an experimental treatment. In certain embodiments, an organoid system is used to detect shifts in the identified cell states to identify agents capable of shifting a subject from a severe disease state to a mild/moderate state (see, e.g., Yin X, Mead B E, Safaee H, Langer R, Karp J M, Levy O. Engineering Stem Cell Organoids. Cell Stem Cell. 2016; 18 (1): 25-38). As used herein, the term “organoid” or “epithelial organoid” refers to a cell cluster or aggregate that resembles an organ, or part of an organ, and possesses cell types relevant to that particular organ. Organoid systems have been described previously, for example, for brain, retinal, stomach, lung, thyroid, small intestine, colon, liver, kidney, pancreas, prostate, mammary gland, fallopian tube, taste buds, salivary glands, and esophagus (see, e.g., Clevers, Modeling Development and Disease with Organoids, Cell. 2016 Jun. 16; 165 (7): 1586-1597). In certain embodiments, a tissue system or tissue explant is used to detect shifts in the identified cell states to identify agents capable of shifting a subject from a severe disease state to a mild/moderate state (see, e.g., Grivel J C, Margolis L. Use of human tissue explants to study human infectious agents. Nat Protoc. 2009; 4 (2): 256-269). In certain embodiments, an animal model is used to detect shifts in the identified cell states to identify agents capable of shifting a subject from a severe disease state to a mild/moderate state (see, e.g., Muñoz-Fontela C, Dowling W E, Funnell S G P, et al. Animal models for COVID-19. Nature. 2020; 586 (7830): 509-515).

In certain embodiments, candidate agents are screened. The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the present invention provides for gene signature screening to identify agents that shift expression of the gene targets described herein (e.g., cell subset markers and differentially expressed genes). The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The gene signatures or biological programs of the present invention may be used to screen for drugs that reduce the signature or biological program in cells as described herein.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science. 1132939; and Lamb, J., The Connectivity Map: a new tool for biomedical research. Nature Reviews Cancer January 2007: Vol. 7, pp. 54-60). In certain embodiments, Cmap can be used to identify small molecules capable of modulating a gene signature or biological program of the present invention in silico.

Further embodiments are illustrated in the following Examples which are given for illustrative purposes only and are not intended to limit the scope of the invention.

### EXAMPLES

### Example 1—LipocyteProfiler

Here, Applicants provide LipocyteProfiler (also referred to herein as Adipocyte Profiler) which is a metabolic disease-orientated phenotypic profiling system for lipid-accumulating cells. LipocyteProfiler expands on CellPainting (Bray M A, Singh S, Han H, et al. Cell Painting, a high-content image-based assay for morphological profiling using multiplexed fluorescent dyes. Nat Protoc. 2016; 11 (9): 1757-1774) and is an unbiased profiling assay, that multiplexes a combination of dyes that make it amenable to large-scale and high-throughput profiling of generic morphological as well as cell type-specific cellular traits. Lipid droplets are storage organelles at the center of whole body metabolism and energy homeostasis and are highly dynamic organelles, that are ubiquitous to cell types (Olzmann and Carvalho 2019) either as part of cellular homeostasis in lipocytes, such as adipocytes, hepatocytes, macrophages/foam cells and glial cells (Liu et al. 2015; Olzmann and Carvalho 2019; Wang et al. 2013; Grandl and Schmitz. 2010; Robichaud et al. 2021) or as part of pathophysiological processes in cells such as vascular smooth muscle cells, skeletal muscle cells, renal podocytes, and cancer cells (Hershey et al. 2019; Cruz et al. 2020; Wang et al. 2005; Weinert et al. 2013; Prats et al. 2006). Applicants vetted LipocyteProfiler in adipocytes, which are highly specialized cells for the storage of excess energy in the form of lipid droplets. First, Applicants connected known biology with rich phenotypic signatures at spatiotemporal resolution, by characterizing feature profiles of known biological processes, including adipocyte differentiation, distinct characteristics of white and brown adipocyte lineages and targeted perturbation of lipid accumulation via CRISPR/Cas9-mediated knockout of specific marker genes, and drug perturbations. Next, Applicants correlated LipocyteProfiles with transcriptomic data from RNAseq to link gene sets with morphological and cellular features that capture a broad range of cell activity in adipocytes. Applicants then used LipocyteProfiler to connect polygenic risk scores for Type 2 Diabetes (T2D)-related traits to intermediate cellular phenotypes, and found trait-specific cellular mechanisms underlying polygenic risk. Finally, Applicants used the method to uncover cellular traits under the genetic control of an individual genetic risk locus, as shown for the 2p23.3 metabolic risk locus at DNMT34. Applicants demonstrated that the customized morphometric approach is capable of identifying diverse cellular mechanisms by generating depot-specific, trait/process-specific and allele-specific morphological and cellular profiles. In the present study, Applicants show the power of LipocyteProfiler to identify genetically informed cellular programs in adipocytes driving metabolic diseases. The approach demonstrated here paves the way to large-scale and high-throughput forward and reverse phenotypic genetic profiling in lipid storing cell types in the future.

### LipocyteProfiler Creates Meaningful Morphological and Cellular Profiles in Adipocytes

To quantitatively map dynamic, context-dependent morphological and cellular signatures in lipocytes and to discover intrinsic and extrinsic drivers of cellular programs, Applicants developed a profiling approach called LipocyteProfiler, based on high-content imaging (FIG. 1a). LipocyteProfiler expands on the CellPainting protocol (Bray et al. 2016) and is an unbiased high-throughput profiling assay, which multiplexes six fluorescent dyes imaged in four channels in conjunction with an automated image analysis pipeline, to generate rich generic and lipocyte-specific cellular profiles (see Methods for more details) (FIG. 1b). LipocyteProfiler extracts 3,005 morphological and cellular features that map to three cellular compartments (Cell, Cytoplasm, Nucleus) across four channels differentiating the organelles, namely DNA (Hoechst), Mito (MitoTracker Red which stains mitochondria), AGP (Phalloidin multiplexed with Wheat Germ Agglutinin, which stain F-actin cytoskeleton, golgi and plasma membranes), and BODIPY (BODIPY multiplexed with SYTO14, which stain neutral lipids, nucleoli and cytoplasmic RNA) (FIG. 1c). To help interpretations of abstract LipocyteProfiler feature signatures, Applicants benchmarked the main classes of feature measurements, namely intensity, granularity and texture in the context of i) adipocyte differentiation (day 0, day 3, day 8, day 14) in human adipose-derived mesenchymal stem cells (AMSCs), which undergo phenotypic changes from fibroblast-shaped to spheric lipid-filled cells during differentiation, ii) directed gene perturbation using CRISPR/Cas9 knockout of known marker genes of adipocyte function and iii) a comparison between cell models for white and brown adipocytes.

Intensity features, which are a collection of features that measure pixel intensities across an image, cover 15.2% of all LipocyteProfiler extracted features. To test if LipocyteProfiler extracts tractable intensity features, Applicants used an established white adipocyte line (hWAT) (Xue et al. 2015) and mapped the phenotypic signature of progressive lipid accumulation over the course of adipocyte differentiation. Applicants showed that intensity of BODIPY, a proxy of overall lipid content within a cell, significantly increases with adipogenic differentiation (FIG. 1d) and confirmed that directed perturbation of PPARG, the master regulator of adipogenesis, using CRISPR/Cas9-mediated knock-out decreases intensity of BODIPY in differentiated white adipocytes (p=4.4e-7, FIG. 1d). Applicants further leveraged intrinsic differences distinguishing white from brown adipocytes, which are known to be predominantly driven by changes in mitochondrial activity (Cedikova et al. 2016), to inform about the information content of mitochondrial features. Using an established brown adipocyte line derived from human neck fat (hBAT) from the same individual as for the hWAT line, Applicants showed that hBAT adipocytes are characterized by increased mitochondrial intensity compared to white (hWAT) adipocytes throughout differentiation, with the most substantial increase in the fully differentiated state (Median, day 3 p=1.9e-2, day 8 p=4.1e-4, day 14 p=2.9e-4; FIG. 1e), indicating that LipocyteProfiler can assign known cellular programs that distinguish different adipocyte lineages. Indeed, when Applicants perturbed PGC1A, the master regulator of mitochondrial biogenesis and thermogenesis in adipocytes, using CRISPR-Cas9 mediated knockout in hWAT, mitochondrial intensity decreased (p=8.0e-4, FIG. 1e), indicating that mitochondrial intensity is a suitable measure of mitochondrial activity.

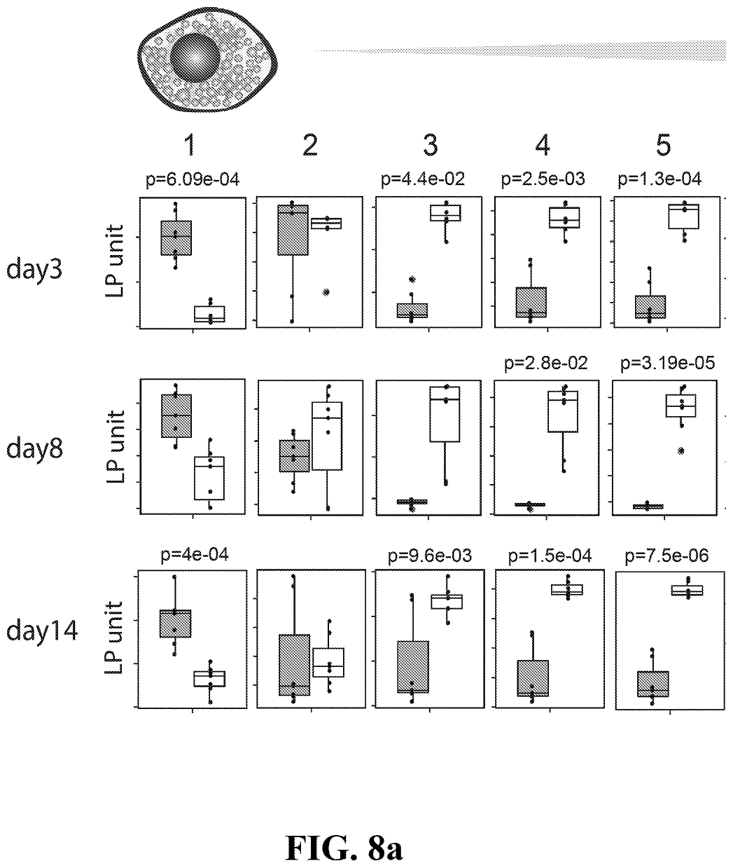

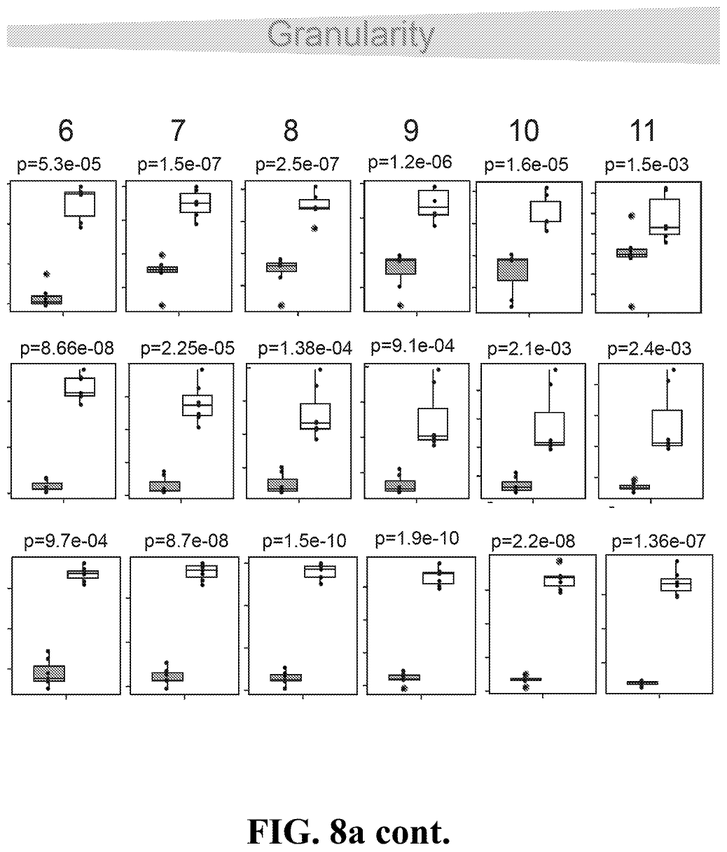

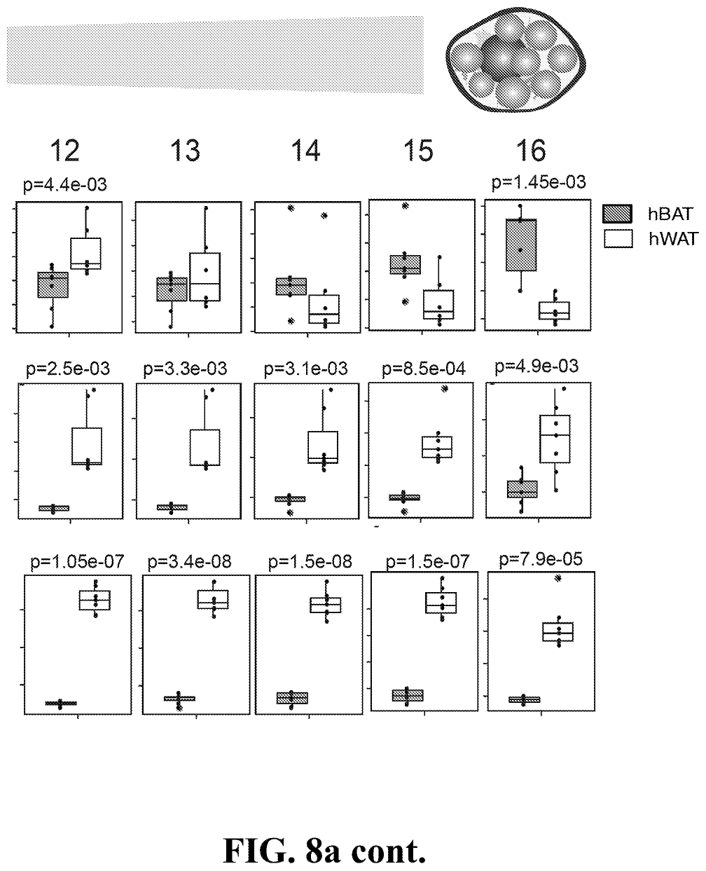

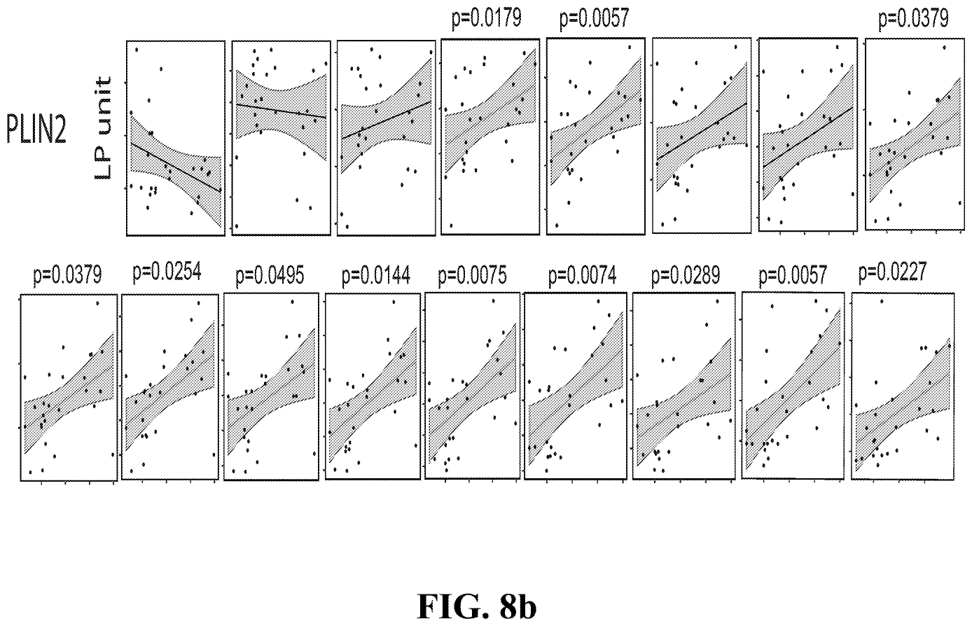

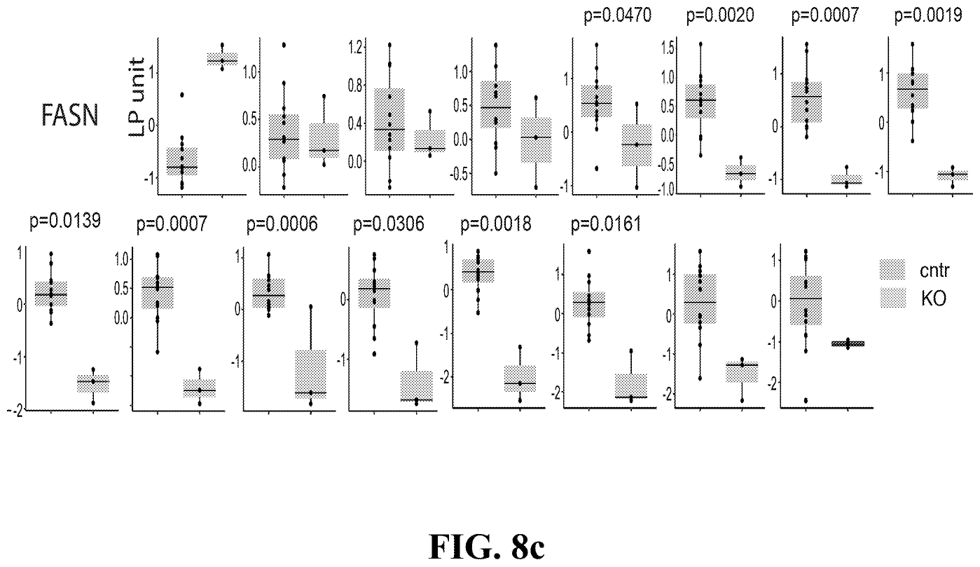

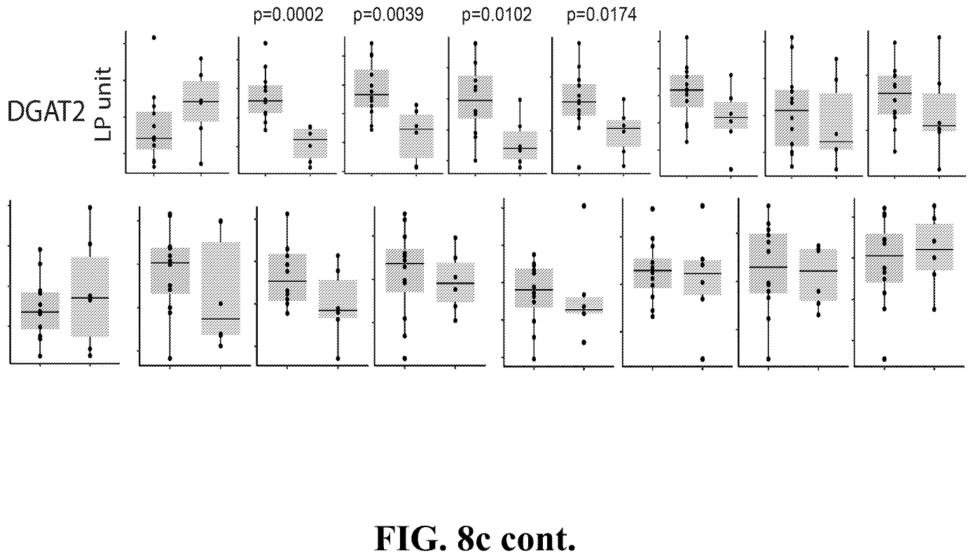

The second class of feature measurement, Granularity, is informative for size spectra and covers 5.9% of total LipocyteProfiler features. Adipocyte differentiation is characterized by the progressive accumulation of lipid droplets that increase first in number and then enlarge and fuse to larger lipid droplets over the course of maturation (Fei et al. 2011). Confirmingly, Applicants found dynamic changes of BODIPY Granularity during the course of differentiation (FIG. 1f). More specifically, Applicants observed that the number of small and medium sized lipid droplets (BODIPY Granularity measures 1-5) present in early differentiating AMSCs either progressively decrease over the course of differentiation or saturate in early stages of differentiation whereas large lipid droplets (BODIPY Granularity measures 10-14) increase in size specifically over the course of differentiation and very large lipid droplets (BODIPY Granularity measures 15-16) are exponentially increasing in terminal differentiation, indicating that lipid droplets form in early differentiation and grow in size thereafter. These data indicate that lipid droplet formation is a dynamic and highly stochastic process that is reflected in BODIPY Granularity measures. To evaluate whether LipocyteProfiler detects intrinsic differences in adipocyte lineages that are known to differ in lipid droplet morphology, Applicants next compared BODIPY Granularity between hWAT and hBAT at day 3, 8 and 14 (FIG. 8a) of adipogenic differentiation. Consistent with the notion that adipocytes from brown adipose tissue have smaller lipid droplets, Applicants found that during differentiation, hBAT generally accumulate less medium-size and large lipid droplets as seen by lower values across the spectra of granularity (FIG. 8a). Applicants then sought to test if lipid droplet size dynamics correlate with mRNA expression levels of lipid droplet-associated perilipins PLIN1, which is specifically expressed in adipocytes and directs the formation of large lipid droplets (Shijun et al. 2020; Gandotra et al. 2011) and PLIN2, which is the only constitutive, ubiquitously expressed lipid droplet protein and associated with a range of lipid droplets in diverse cell types (Brasaemle et al. 1997; Tsai et al. 2017). Applicants observed that mRNA expression levels of PLIN1 positively correlated with BODIPY Granularity features informative for larger lipid droplets (BODIPY_Gramilarity 12-16) (FIG. 1f). PLIN2 correlated with BODIPY Granularity measures of the smaller and larger size spectra (significant for BODIPY_Granularity 4-5 and BODIPY_Granularity 8-16) (FIG. 8b). Confirmingly, when Applicants knocked-out PLIN1, FASN, and DGAT, genes involved in lipid droplet dynamics and lipid metabolism, Applicants observed a size-specific reduction of BODIPY Granularity (FIG. 1f, FIG. 8c), suggesting that BODIPY Granularity features are a suitable output measure of lipid droplet size spectra and an indicator of adipocyte differentiation.

The third main class of features are Texture features (67.8% of total features) that describe the complexity within an image. During adipogenesis of hWAT, AGP Texture_AngularSecondMoment, a measure for image homogeneity, was decreased, whereas it was increased for BODIPY (FIG. 1g). In contrast, Texture_Entropy had the inverse direction (FIG. 1g), suggesting that adipocyte differentiation, a process which is accompanied by drastic cytoskeletal remodelling (more specifically the break-down of F-actin) is reflected by an increase in AGP stain complexity and a less uniform appearance, whereas the lipid droplet-related profile becomes more homogenous as cells mature. When comparing BODIPY Texture in hBAT and hWAT at day 8 of differentiation, Applicants found that hWAT cells showed a more homogenous lipid droplet-related appearance than hBAT (p=0.002, BODIPY Texture AngularSecondMoment; p=0.0126 BODIPY Texture Entropy; FIG. 1g). For mitochondrial stains, LipocyteProfiler derived Texture features can be informative for mitochondrial dynamics, including fission and fusion events. This was evident as perturbation of MFN1, a mitochondrial fusion gene, changes Mitochondria_Texture_InfoMeas1, a measure of spatial relationship between specific intensity values (FIG. 1h; p=0.0091).

LipocyteProfiler extracts a fourth class of Other features, which reflect various measurements of Area, Shape and Size. These size estimates intuitively change over the course of differentiation as cells become lipid-laden, grow in size, and as nuclei become more round and compact (FIG. 1j). For instance, LipocyteProfiler allows to effectively extract quantitative measures of large BODIPY objects informative for large lipid droplets, which are absent in the progenitor state (day 0) and in early differentiation (day 3), increase in later stages of differentiation and are reduced when perturbing the regulators of lipid accumulation, PPARG and PLIN1, at day 14 of differentiation (p=7.3e-09, PPARG-KO; p=7.8e-04, PLIN1-KO; FIG. 1i). Together, LipocyteProfiler outputs a rich set of morphological and cellular features that correlate with cellular function and allow identification of generic and lipocyte-specific morphological and cellular features.

### LipocyteProfiles Reflects Transcriptional State in Adipocytes

To identify relevant processes that converge into morphological and cellular features and to identify pathways of a given set of features, Applicants next used a linear mixed model to correlate the expression of 60,000 genes derived from RNAseq with each of the 2,760 image-based features derived from LipocyteProfiler in adipocytes at day 14 of differentiation. Applicants found 44,736 non-redundant significant feature-gene connections (FDR<0.1) that were composed of 10,931 genes and 869 features, that mapped across all channels (FIG. 2a). Although features from every channel had significant gene correlations, BODIPY features showed the highest amount of gene connections compared to any other channel, suggesting that lipid droplet structure, localization and dynamics in adipocytes most closely represent the transcriptional state of the cell (FIG. 2b). Pathway enrichment analyses of gene-feature connections confirmed that the genes that correlated with a particular feature were biologically meaningful. For example, mitochondrial granularity as a measure of mitochondrial dynamics, was enriched for genes involved in the tricarboxylic acid cycle (TCA) which oxidizes acetyl-CoA in mitochondria and BODIPY intensity as a measure of overall lipid content was enriched for genes involved in oxidative phosphorylation (OXPHOS) and beta-oxidation. Similarly, BODIPY Granularity as a measure of lipid droplet sizes was enriched for adipogenesis, apoptosis and differentiation of white and brown adipocytes and a Correlation feature that measures overlap between lipid droplets, mitochondrial and AGP stains was enriched for cytoplasmic ribosomal protein and beta-oxidation pathway (FIG. 2b; Table 1). In reverse, Applicants found that morphological signatures of adipocyte marker genes SCD, PLIN2, LIPE, INSR, GLUT4 and TIMM22 recapitulate their cellular function (FIG. 2c; Table 2). For example, TIMM22, a mitochondrial membrane gene, showed highest positive correlations with mitochondrial Texture and Intensity features suggesting that mitochondrial Texture describes mitochondrial structures and mitochondrial Intensity describes mitochondrial membrane potential in adipocytes. Together these data show that the mechanistic information gained from LipocyteProfiles is not limited to generic cellular organelles but reflects a transcriptional state of the cell and can be deployed to gain relevant mechanistic insight.

LipocyteProfiler Identifies Distinct Depot-Specific Morphological Signatures Associated with Differentiation Trajectories in Both Visceral and Subcutaneous Adipocytes

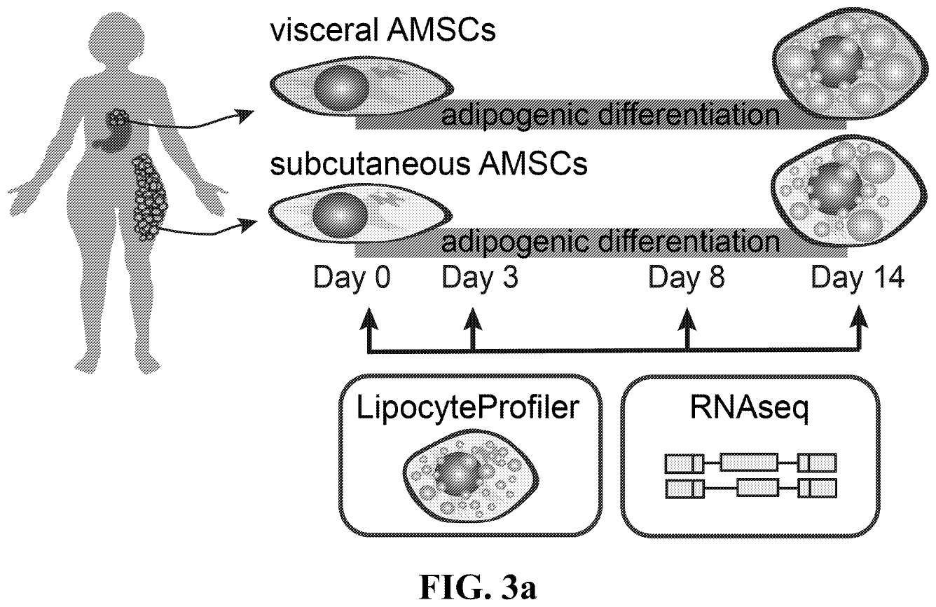

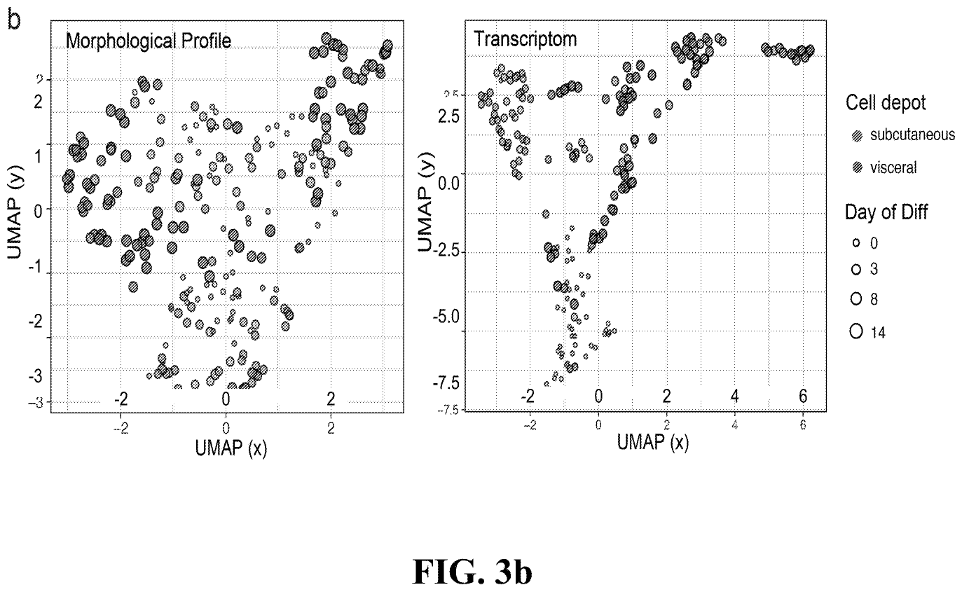

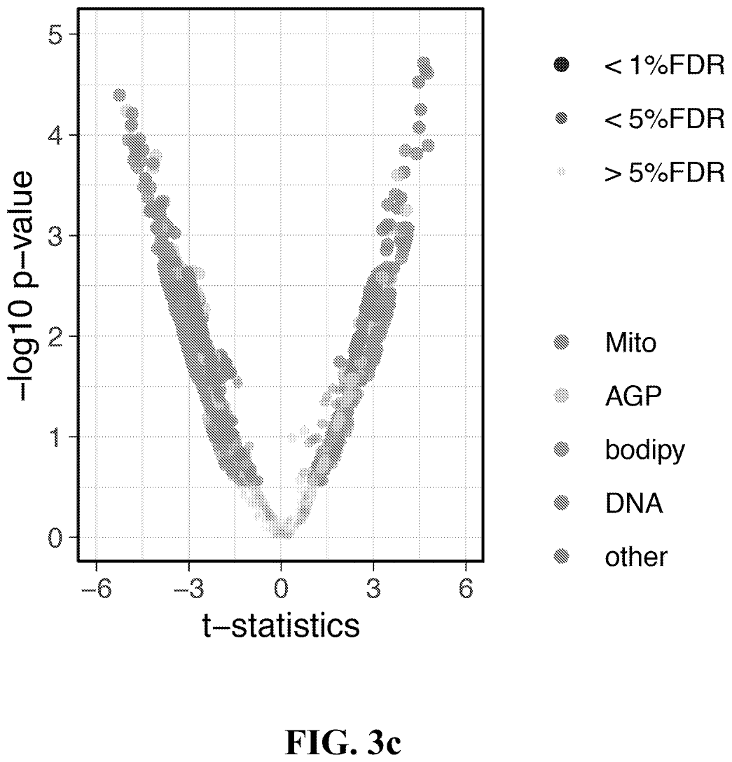

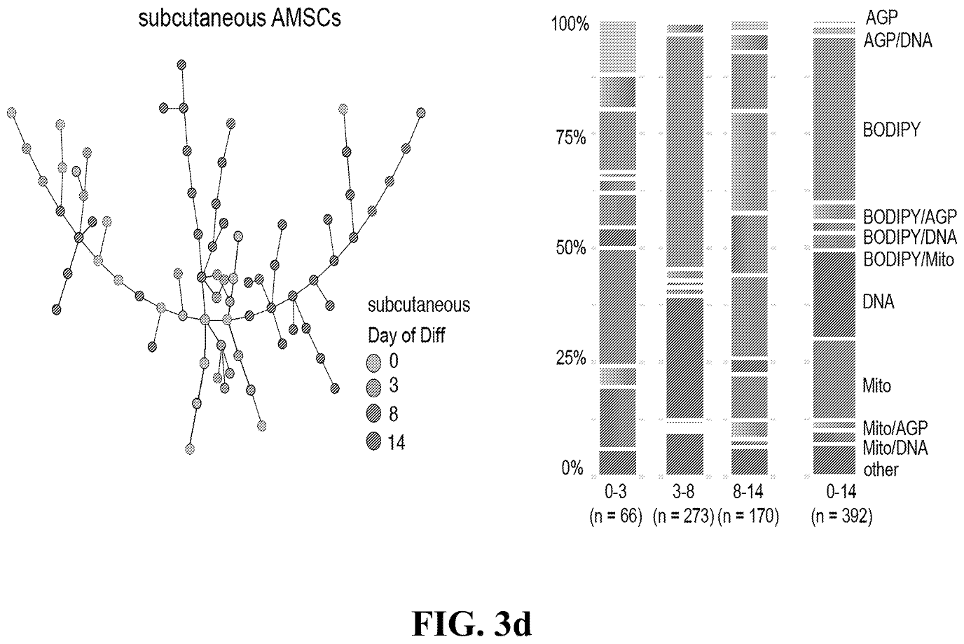

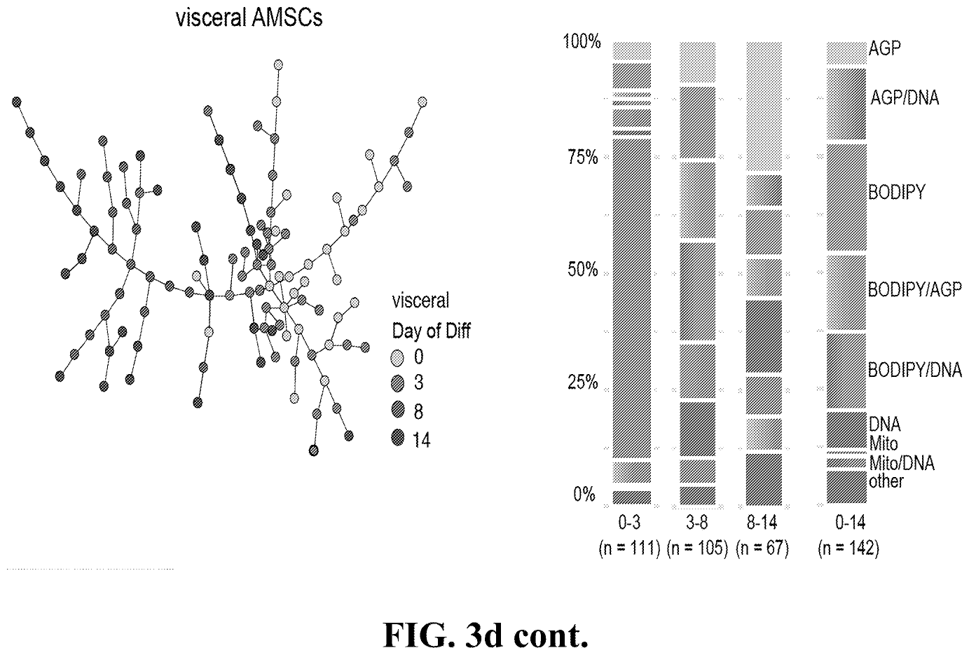

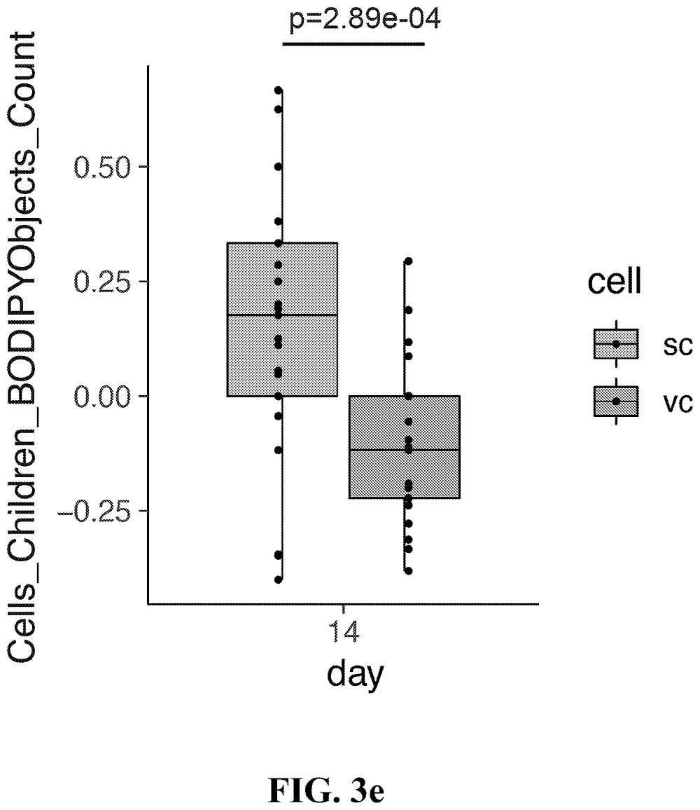

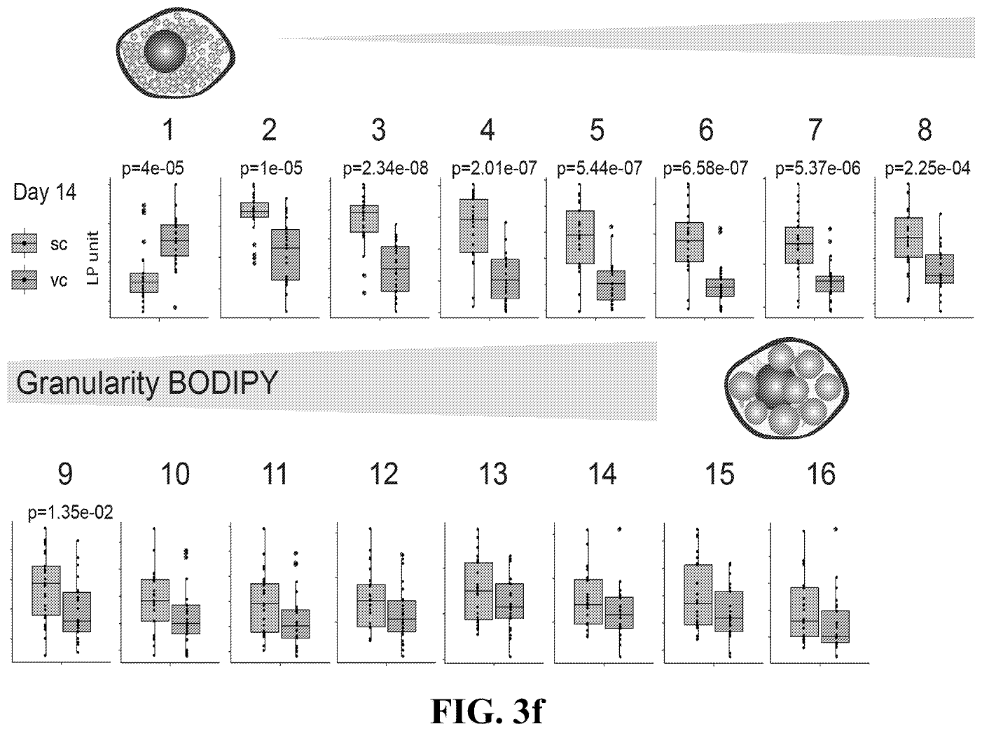

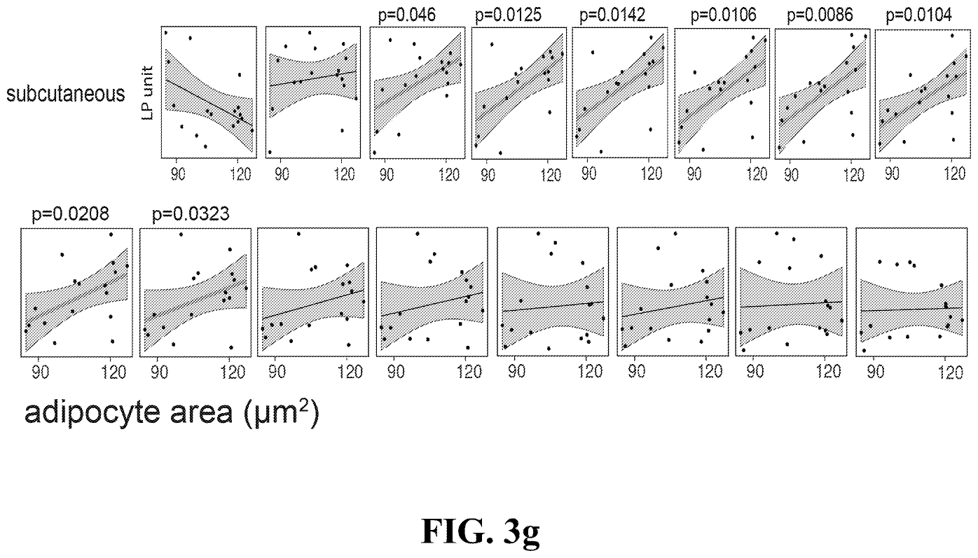

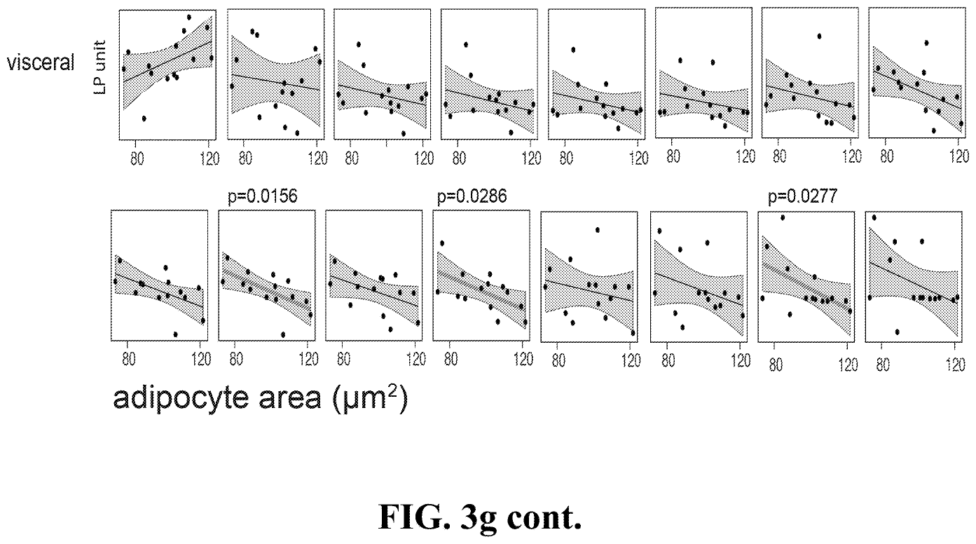

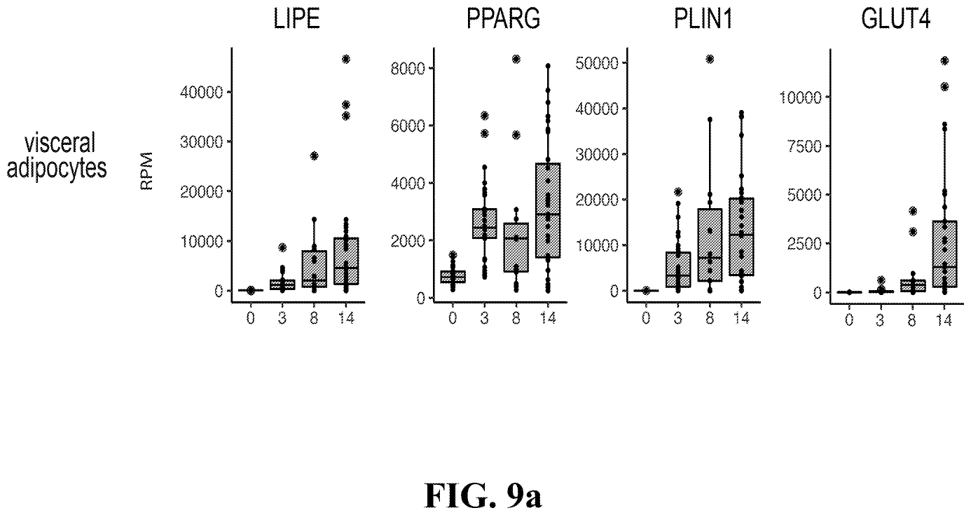

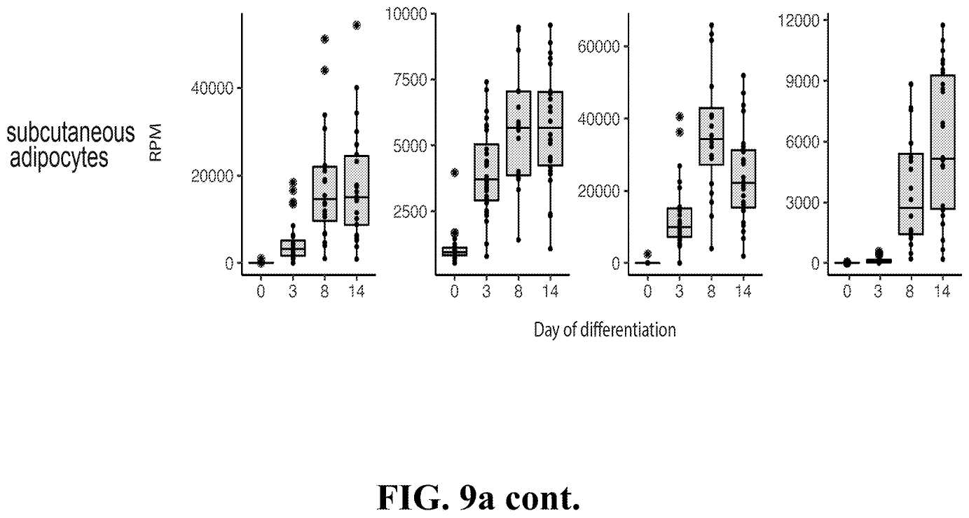

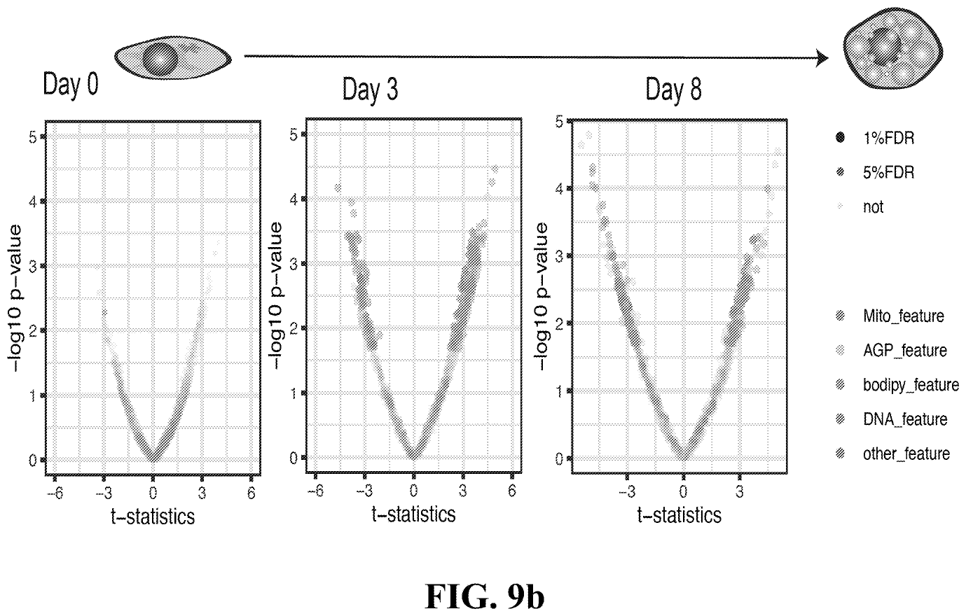

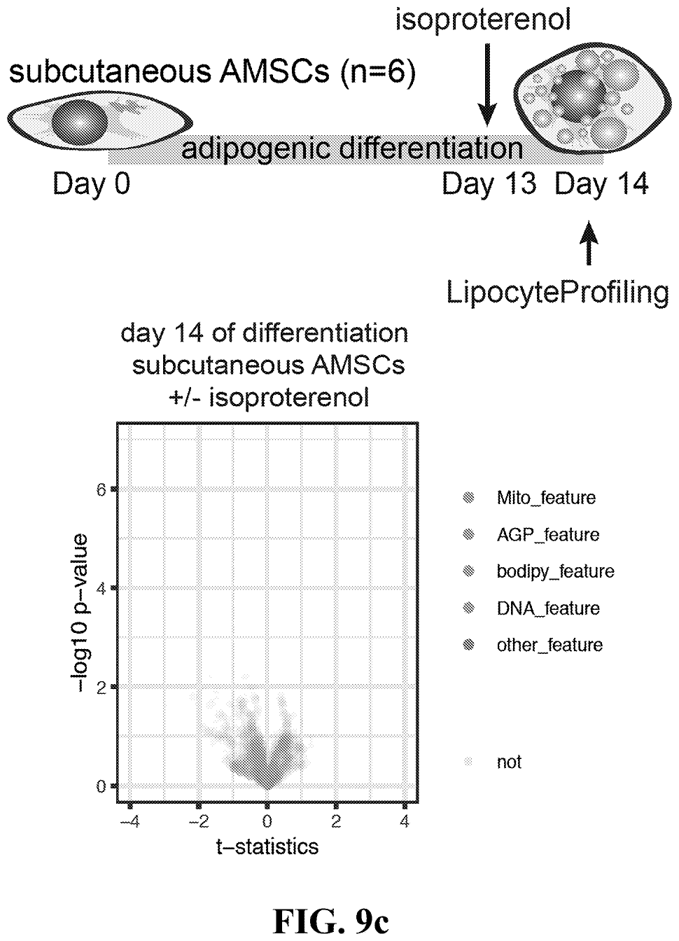

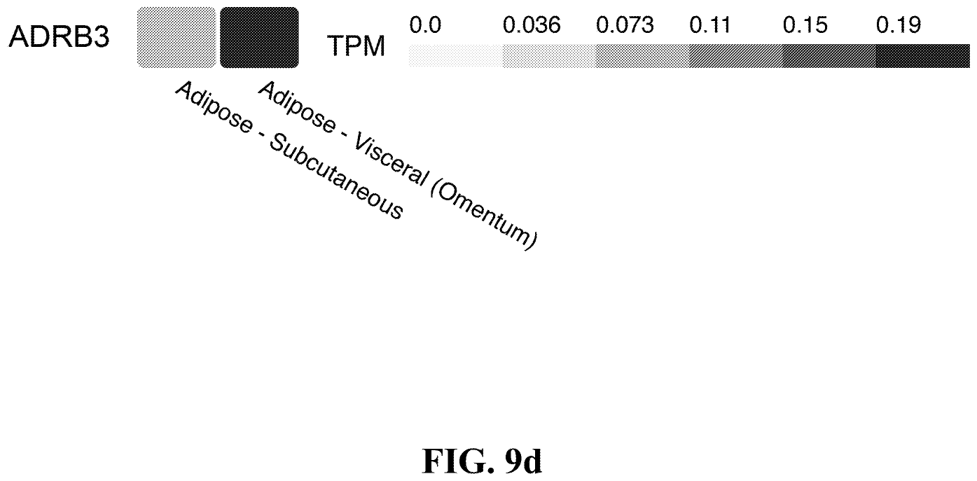

Applicants next sought to distinguish primary human AMSCs derived from the two main adipose tissue depots in the body, namely subcutaneous and visceral, across the course of differentiation. Applicants used those profiles to resolve adipogenesis into temporal dynamic changes in cell morphology (FIG. 3a). Applicants differentiated subcutaneous and visceral AMSCs for 14 days, acquired cell images cells at day 0, day 3, day 8 and day 14 using LipocyteProfiler and validated successful differentiation in both depots by an increase of adipogenesis marker genes (LIPE, PPARG, PLIN1, GLUT4) (FIG. 9a). Concomitantly, Applicants performed RNA-sequencing based transcriptomic profiling at the same differentiation days. Applicants observed that both the morphological and transcriptomic profiles show time course-specific signatures revealing a differentiation trajectory, but only morphological profiles generated by LipocyteProfiler additionally resolve adipose depot-specific signatures throughout differentiation that are spread across all feature classes (FIG. 3b-c, 10b). To discover patterns of adipogenic progression across depot-specific adipogenic differentiation Applicants performed a sample progression discovery analysis (SPD) (Qiu et al. 2011). SPD clusters samples in a manner that reveals their underlying progression and simultaneously identifies subsets of features that show the same progression pattern and are driving differentiation. Applicants discovered that subsets of features driving differentiation differ between subcutaneous and visceral adipocytes and that most dominant feature classes are dynamically changing over the time course of differentiation (FIG. 3d). In visceral adipocytes, mitochondrial features drive differentiation predominantly in the early phase of differentiation whereas BODIPY-related features predominate in the terminal phases (FIG. 3d). In subcutaneous adipocytes, Applicants observed that all feature classes (actin-cytoskeleton, lipid, mitochondrial and nucleic-acid) are equally involved across adipogenesis and that contribution of BODIPY features starts in early phases of differentiation, revealing that accumulation of lipids starts earlier in subcutaneous compared to visceral adipocytes (FIG. 3d). Applicants next compared BODIPY-related depot-specific signatures in mature AMSCs and observed that subcutaneous AMSCs have more lipid droplets compared to visceral AMSCs (BODIPY_Object_count, FIG. 3e p=2.89e-4). More specifically, mature subcutaneous AMSCs show significantly higher BODIPY Granularity of small to medium size granularity measures, whereas visceral adipocytes show higher granularity of very small granularity size measures, suggesting that mature subcutaneous AMSCs have larger intracellular lipid droplets compared to visceral which present more very small and less-defined lipid droplets (FIG. 3f). Expression levels of marker genes of mature adipocytes LIPE, PPARG, PLIN1 and GLUT4 are lower in visceral compared to subcutaneous AMSCs (FIG. 9a). Those results reveal that adipose depots have intrinsically different differentiation capacities and lipid accumulation programs which is in line with previously described distinct properties of subcutaneous and visceral AMSCs across differentiation (Baglioni et al. 2012). This data suggests that LipocyteProfiler is capable of distinguishing morphological and cellular profiles of AMSCs derived from different adipose depots and can facilitate identifying distinct cellular programs driving differentiation that show visible differences in subcutaneous compared to visceral AMSCs.

Lastly, to assess the in vivo relevance of morphological features of in vitro differentiated adipocytes, Applicants correlated BODIPY Granularity features with tissue-derived size estimates of mature floating adipocytes. Applicants showed that changes of BODIPY Granularity of in vitro differentiated female subcutaneous adipocytes correlate significantly with the mean adipocyte size from tissue (FIG. 3g). More specifically, medium size granularity measures increase with larger in vivo size estimates, suggesting that in vivo adipocyte size is reflected by more medium sized lipid droplets in in vitro differentiated subcutaneous adipocytes. Strikingly, Applicants find the opposite direction of effect between correlation of visceral BODIPY Granularity and tissue-derived adipocyte size estimates from floating adipocytes, suggesting that subcutaneous and visceral adipose tissue differ in their cellular programs that govern depot-specific adipose tissue expansion and may account for different depot-specific susceptibility to metabolic diseases. Indeed, white adipose depots have been reported to differ in their respective mechanisms of fat mass expansion under metabolic challenges, with subcutaneous adipose tissue being more capable of hyperplasia whereas visceral adipose tissue expands mainly via hypertrophy (Wang et al. 2013).

### LipocyteProfiler Reveals Cellular Mechanisms Underlying Drug Perturbations in Adipocytes and Hepatocytes

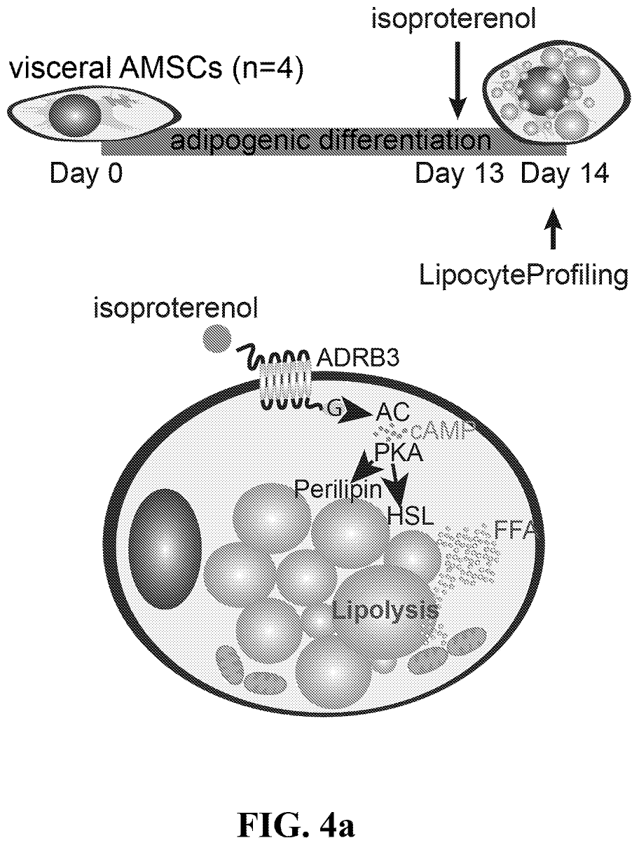

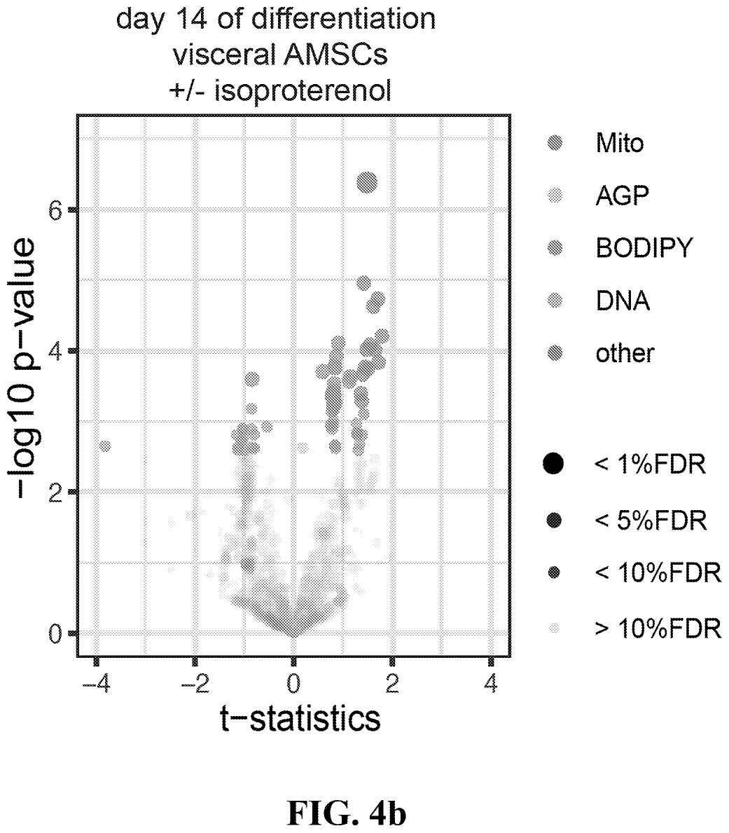

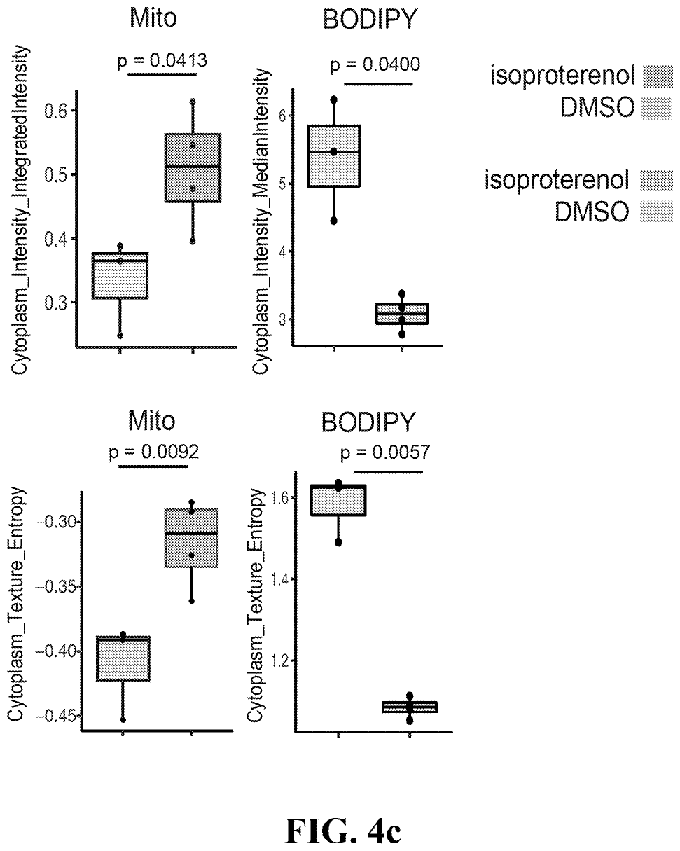

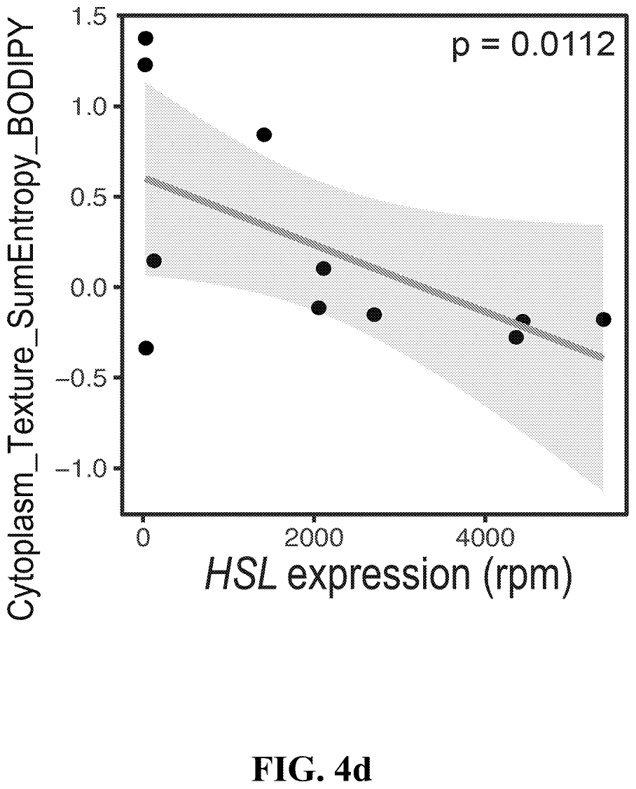

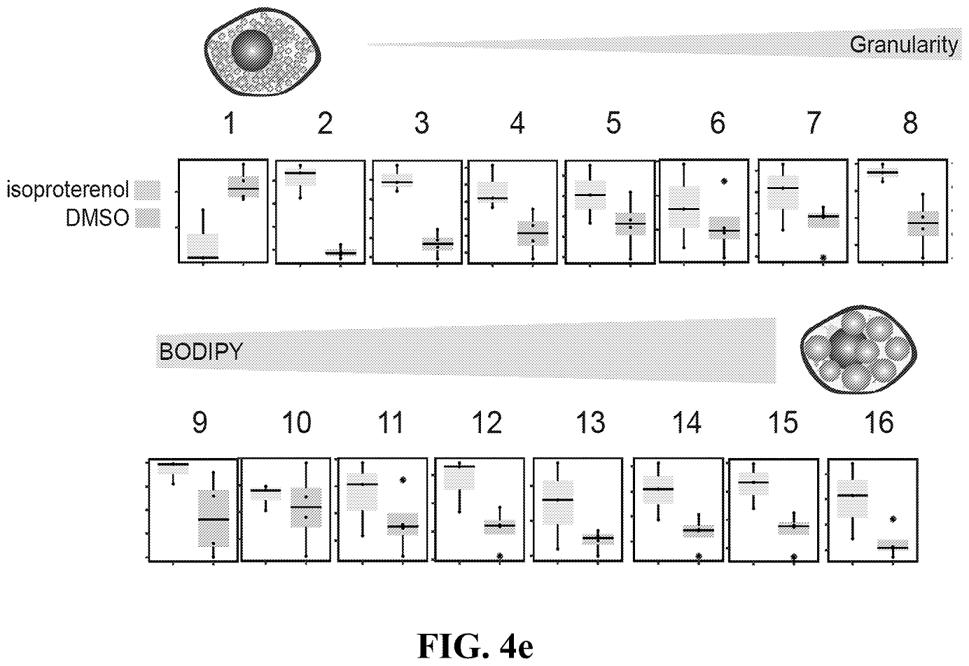

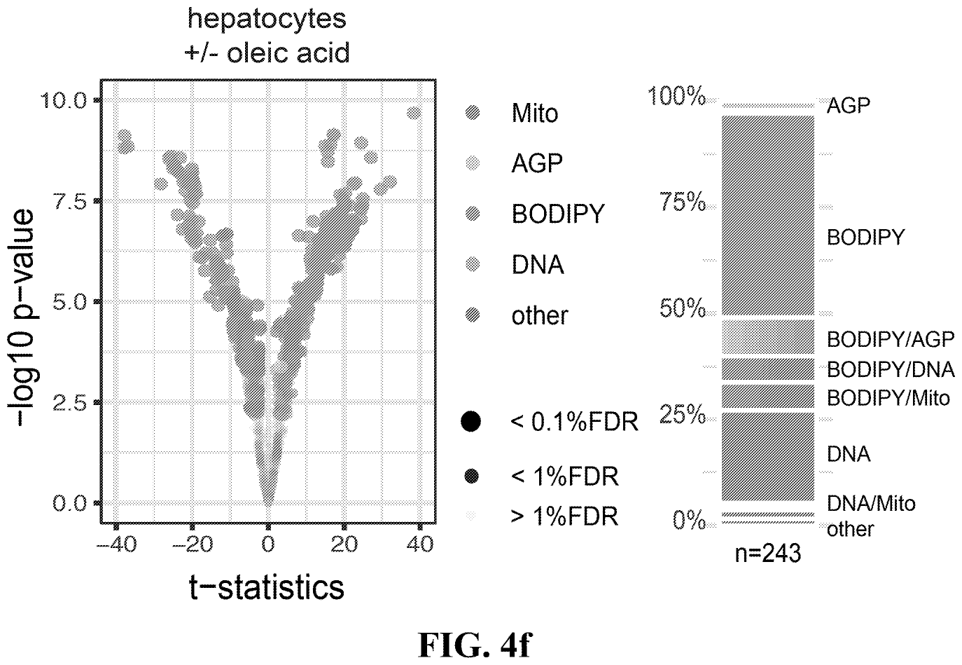

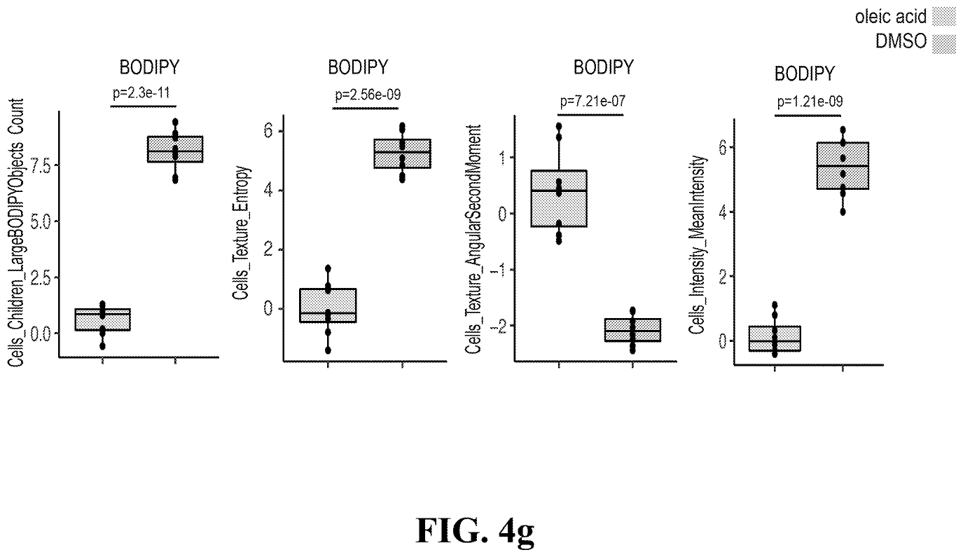

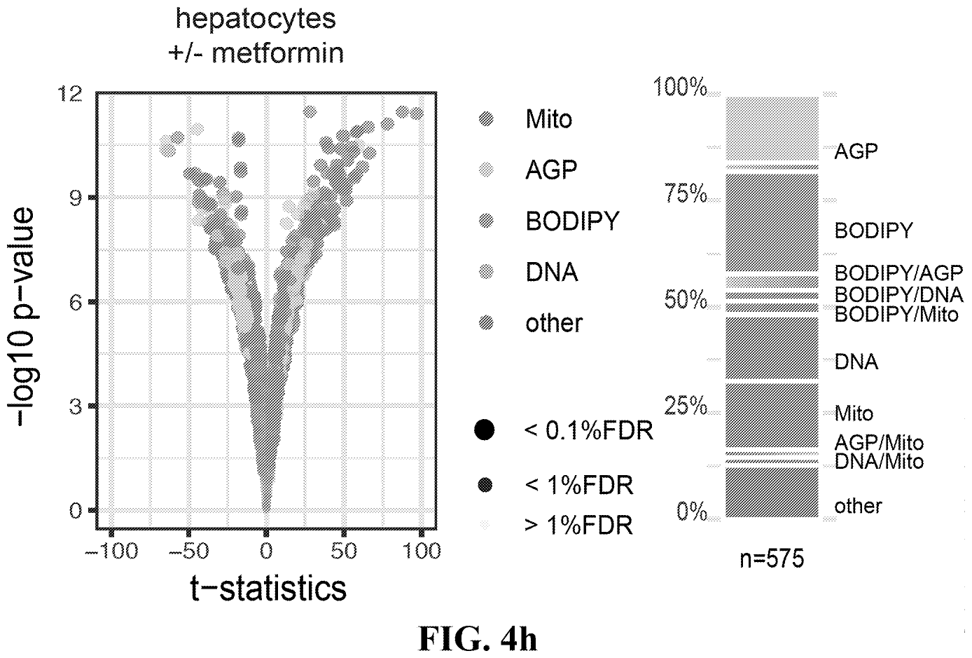

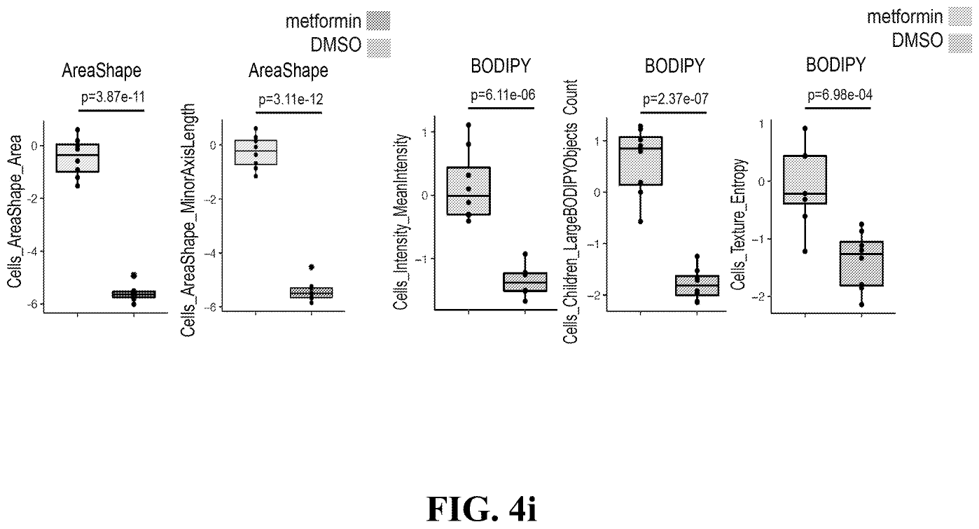

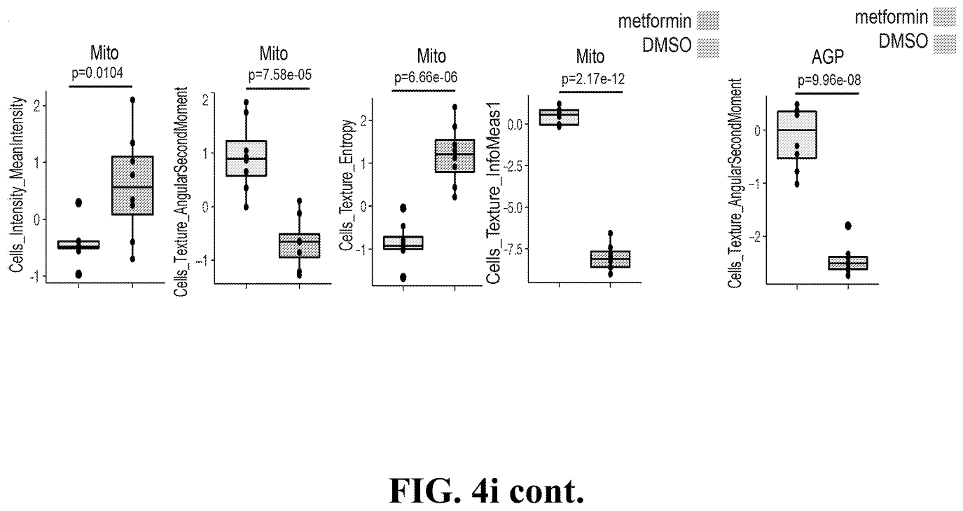

To investigate whether LipocyteProfiler is capable of identifying effects of drug perturbations on morphological and cellular profiles, Applicants first assayed isoproterenol-stimulated compared to DMSO-treated subcutaneous and visceral adipocytes. Isoproterenol is a β-adrenergic agonist that binds to the β-adrenergic receptor (ADRB) in adipocytes. While isoproterenol is known to induce lipolysis and increase mitochondrial energy dissipation (Miller et al. 2015), Applicants set out to find out whether its concerted effects on morphological and cellular signatures could be captured using LipocyteProfiler (FIG. 4a). Applicants observed that visceral adipocytes respond to 24 hour isoproterenol treatment by changes in BODIPY and Mito features (FIG. 4b, Table 3). More specifically, Applicants observed that isoproterenol-treated visceral adipocytes had increased Mitochondrial Intensity (p=0.0413) and differences in mitochondrial Texture_Entropy (p=0.0092), indicative of a more complex appearance compared to DMSO-treated controls (FIG. 4c) and suggesting that isoproterenol treatment results in more active mitochondria in the fission process, which is a reported mechanism of norepinephrine-stimulated browning in adipocytes (Gao and Houtkooper 2014). Isoproterenol-treated visceral adipocytes are further characterized by decreased BODIPY MedianIntensity (p=0.041) and Texture Entropy (p=0.032) (FIG. 4c) as well as decreased BODIPY-related Granularity across the full granularity size spectra (FIG. 4d), suggesting smaller lipid droplets with less overall lipid content in isoproterenol-treated visceral adipocytes compared to DMSO-treated controls due to increased lipolysis. Indeed, hormone sensitive lipase (HSL), a marker of lipolysis, correlated with BODIPY Texture Entropy in visceral adipocytes (p=0.0122; FIG. 4d). Finally, the phenotypic response following isoproterenol treatment was specific to visceral adipocytes, as isoproterenol perturbation had no effect in subcutaneous adipocytes (FIG. 9c). Concordantly, ADRB expression is higher in visceral compared to subcutaneous adipose tissue (FIG. 9d).

Next, Applicants assayed the effects of oleic acid and metformin in primary human hepatocytes (PHH) using LipocyteProfiler. It has been shown that free fatty acid treatment induces lipid droplet accumulation in PHH (Liu et al. 2014). The results showed that 24h treatment of PHH with oleic acid (OA) resulted in changes predominantly of BODIPY features (FIG. 4f, Table 3), with a morphological profile indicative of increased lipid droplet number (LargeBODIPYObjects_Count, p=2.3e-11) and overall lipid content (BODIPY MeanIntensity, p=1.21e-09) as well as differences in Texture (BODIPY Texture Entropy, p=2.56e-09; BODIPY Texture_AngularSecondMoment, p 7.21e-07; FIG. 4g). Alternatively, treatment of PHH with 5 mM metformin for 24h, on the other hand, caused morphological and cellular changes that were spread across all channels (FIG. 4h, Table 3), with a profile suggestive of smaller cells (AreaShape Area, p=3.87e-11; AreaShape MinorAxislength, p=3.11e-12) with increased mitochondrial membrane potential (Mito MeanIntensity, p 0.0104) due to increased fusion (Mito Texture_angularSecondMoment, p=7.58e-05; Mito Texture Entropy, p=6.66e-06; Mito Texture_InfoMeas1, p=2.17e-12) and reduced lipid content (BODIPY MeanIntensity, p=6.11e-06), reduced lipid droplet number (LargeBODIPYObjects_Count, p=2.37e-07) and difference in Texture (BODIPY Texture Entropy, p 6.98e-04) (FIG. 4i). This concerted effect of metformin on mitochondrial dynamics and function as well as lipid-related features is consistent with a less uniform appearance of the cytoskeleton, Golgi and plasma membrane in metformin-treated hepatocytes compared to control (AGP Texture_AngularSecondMoment, p=9.96e-08). Indeed, metformin is known to inhibit mitochondrial complex 1 in hepatocytes which increases mitochondrial membrane potential and leads to diminished lipid accumulation in primary hepatocytes (Liu et al. 2014). Together, Applicants showed that morphological and cellular profiles of drug perturbation in lipocytes resemble an amelioration of cellular signatures of known biology and drug action in a single concerted snapshot of cell behavior.

### Polygenic Risk Effects for Insulin Resistance Converges on a Lipid Rich Morphological Profile in Differentiated Visceral Adipocytes

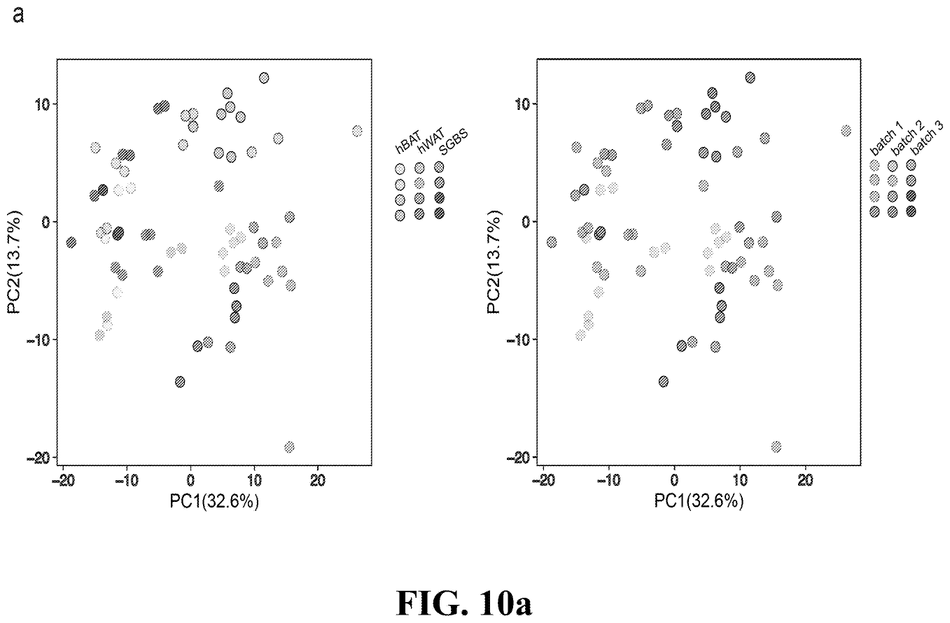

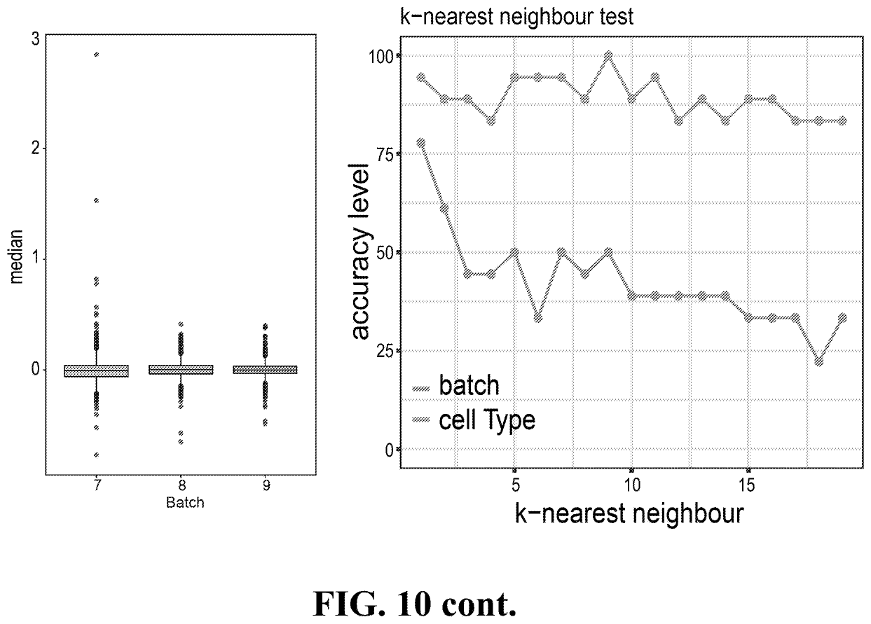

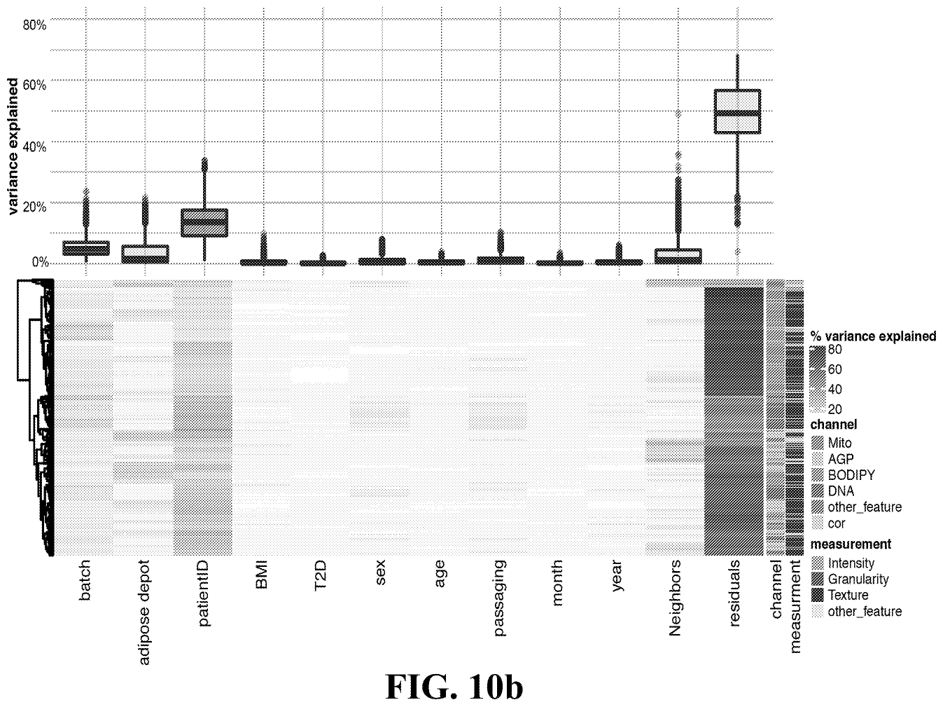

Next, Applicants used LipocyteProfiler to discover cellular programs of metabolic polygenic risk in adipocytes. For systematic profiling of AMSCs in the context of natural genetic variation (Table 4), Applicants first assessed the effect of both technical and biological variance on LipocyteProfiler features in the setting. To obtain a measure of batch-to-batch variance associated with the experimental set-up, Applicants differentiated hWAT, hBAT and SGBS preadipocytes (Fischer-Posovszky et al. 2008) in three independent experiments and found no significant batch effect (BEscore 0.0047, 0.0001, 0.0003, FIG. 10a). Applicants also showed that the accuracy of predicting cell type is higher than predicting batch (FIG. 10a), indicating that the LipocyteProfiler framework is associated with low batch effect and high accuracy to detect intrinsic versus extrinsic variance within the data set. Secondly, Applicants performed a variance component analysis across all data, on adipocyte morphological and cellular traits across 65 donor-derived differentiating AMSCs, to assess the contribution of intrinsic genetic variation compared to the contribution of other possible confounding factors such as batch, adipose depot, T2D status, age, sex, BMI, cell density, month/year of sampling and passage numbers. In total Applicants found that across all samples and batches, the largest contributor to feature variance was donor ID, explaining 11.45% (25th quantile)-21.95% (75th quantile) (median 17.03%) of variance (FIG. 10b). Other factors appeared to contribute only marginally to overall variance of the data, including batch effect (3.94%-8.84%, median=6.02%), adipose depot (7.68%-7.12%, median-2%) and plating density (3.75%-5.61%, median=1.55%). This suggests that LipocyteProfiler is able to detect and distinguish inter-individual genetic variation on features to a similar degree as reported for human iPSCs, where quantitative assays of cell morphology demonstrated a donor contribution to inter-individual variation in the range of 8%-23% (Kilpinen et al. 2017). However, to account for the variable feature-specific contributions of batch, sex, age and BMI to overall feature variance, Applicants forthon corrected for those covariables in the analyses. Together, these data suggest that AMSCs-derived LipocyteProfiles can be used to study the effect of genetic contributions to cellular morphology.

Using the latest summary statistics for T2D, Applicants then constructed individual genome-wide polygenic risk scores (PRSs) for three T2D-related traits that have been linked to adipose tissue, using the latest summary statistics for T2D (Mahajan et al. 2018), HOMA-IR (Dupuis et al. 2010), a proxy of insulin resistance (Matthews et al. 1985), and waist-to-hip ratio adjusted for BMI (WHRadjBMI) (Pulit et al. 2019). Applicants selected donors from the bottom and top 25 percentiles of these genome-wide PRSs (forthon referred to as low polygenic risk and high polygenic risk, respectively) and compared LipocyteProfiles across the time course of visceral and subcutaneous adipocyte differentiation in high and low polygenic risk groups for each of the traits (FIG. 5a).

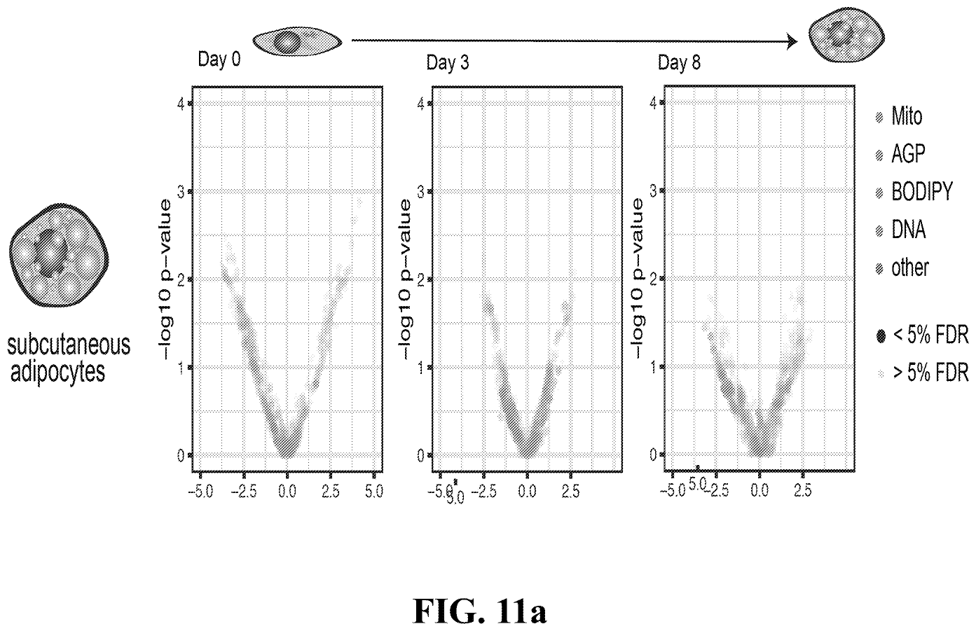

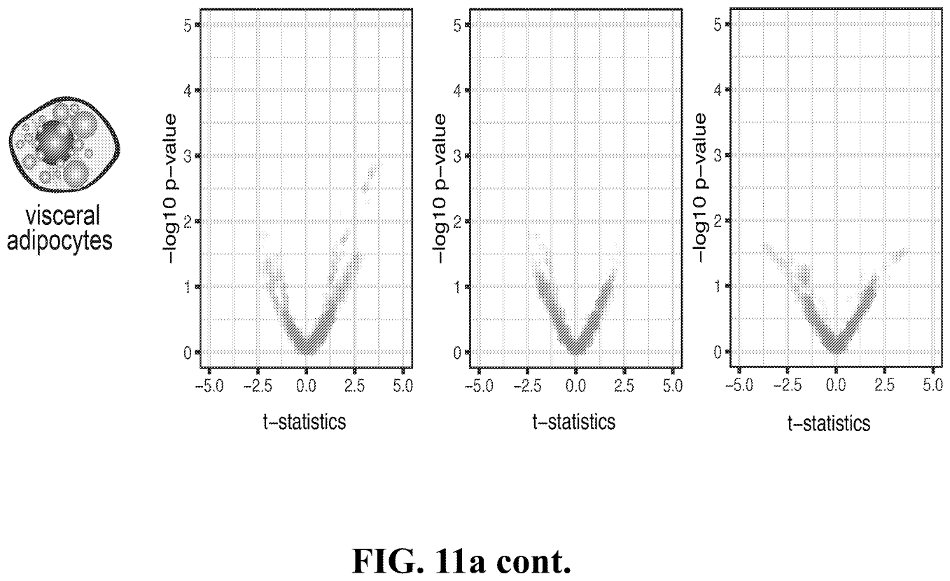

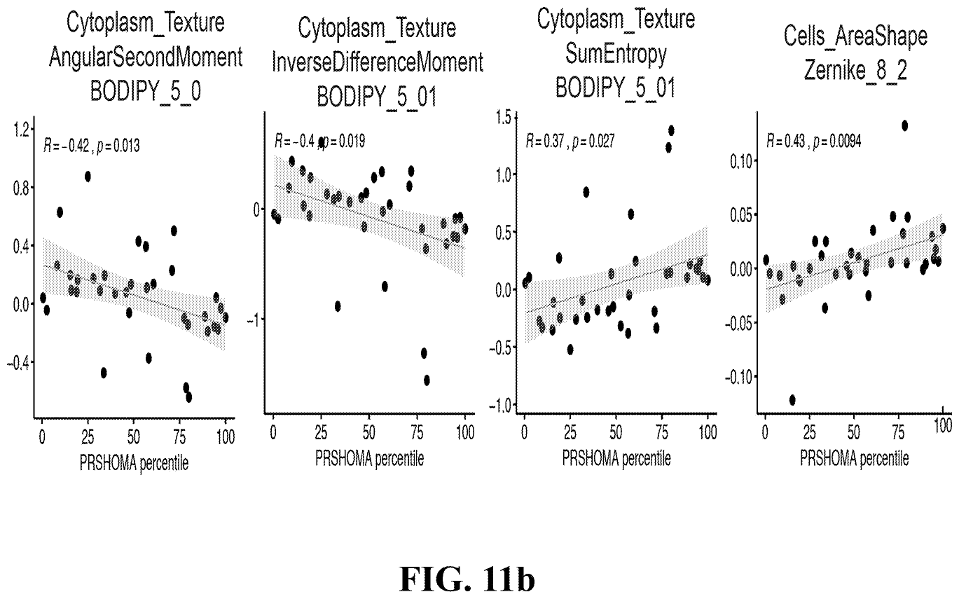

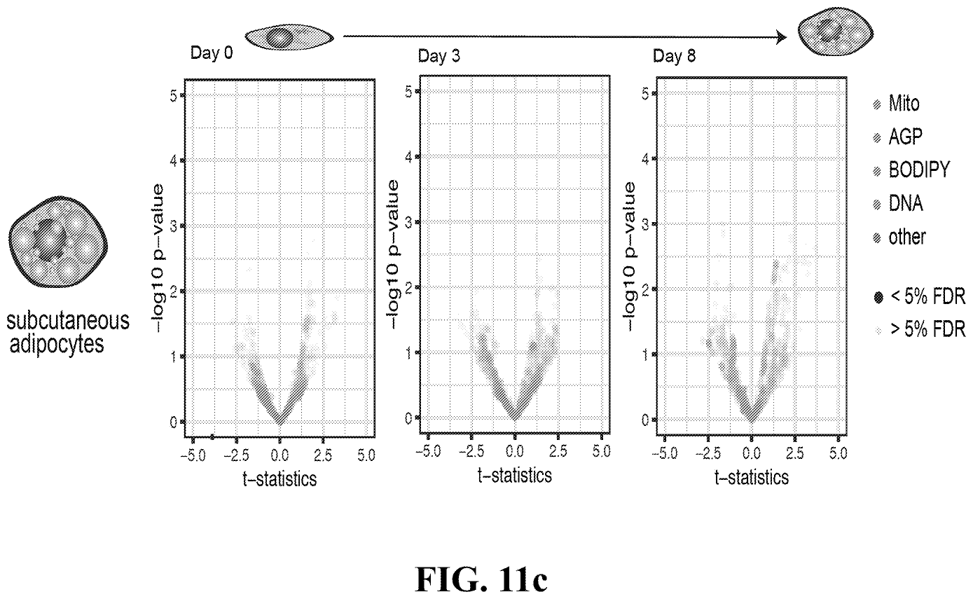

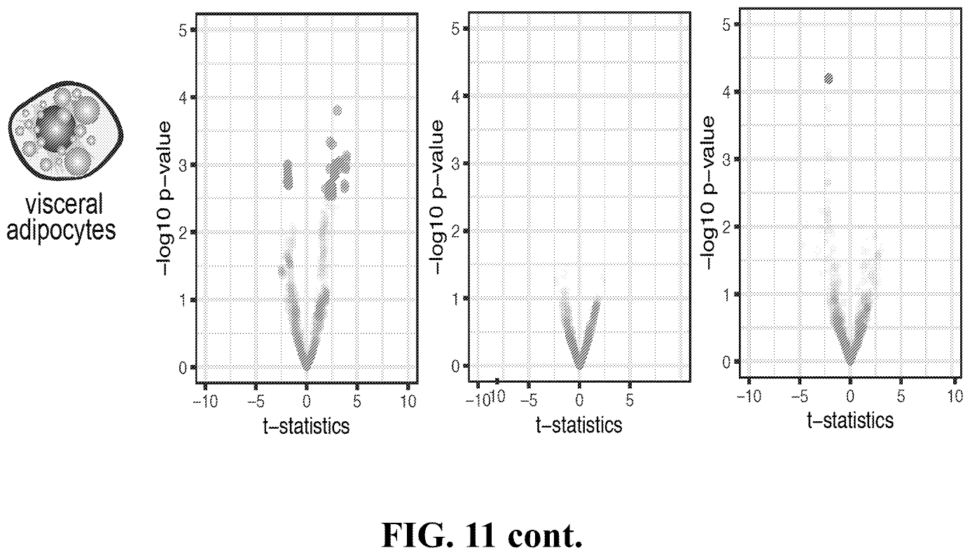

Applicants found significant polygenic effects on image-based cellular signatures for HOMA-IR and WHIRadjBMI, but no effect for T2D (Table 5). More specifically, Applicants observed an effect of HOMA-IR polygenic risk on morphological profiles at day 14 in visceral adipocytes (43 features, FDR<5%, FIG. 5b, FIG. 11a-d), indicating a spatiotemporal and depot-specific effect of polygenic risk. The features different in the high compared to low HOMA-IR PRS carriers mapped mostly to the BODIPY channel (FIG. 5b), where visceral adipocytes from high polygenic risk individuals showed increased BODIPY Granularity (p=4.6E-04, FIG. 5c), increased Texture_SumEntropy (p=2.7E-03, FIG. 5c), increased Area_Shape (p=9.3E-03, FIG. 5c), decreased Texture_InverseDifferenceMoment (p=1.5E-03, FIG. 5c) and reduced Texture_AngularSecondMoment (p=4.6E-05, FIG. 5c). This profile recapitulates signatures that resemble an inhibition of lipolysis and lipid degradation, as shown with the inverse direction of effect in isoproterenol-stimulated visceral AMSCs as shown in FIG. 4, and indicate that visceral adipocytes from individuals with high polygenic risk for insulin resistance show a heterogeneous lipid-associated morphological profile, with differences in lipid droplet size, number and distribution, and coherent with excessive lipid accumulation due to a decreased degradation of lipids by lipolysis. Applicants further ascertained the effects of polygenic risk for HOMA-IR on gene expression of 512 genes known to be involved in adipocyte differentiation and function, and identified 51 genes under the polygenic control of HOMA-IR (FDR<10%) in fully differentiated visceral adipocytes (FIG. 5d, Table 6). Applicants observed that genes, which correlate with the HOMA-IR PRS were enriched for biological processes related to glucose metabolism, fatty acid transport, degradation and lipolysis (FIG. 5e, Table 7). Negatively correlated genes include ACAA1 (p=1.58E-02) and SCP2 (p=9.41E-04) (FIG. 5f), coherent with an inhibition of lipolysis and lipid degradation in visceral adipocytes from individuals at high polygenic risk for HOMA-IR. Positively correlated genes include GYS1, a regulator of glycogen biosynthesis, which has been shown to causally link glycogen metabolism to lipid droplet formation in brown adipocytes (Mayeuf-Louchart et al. 2019) (p=5.48E-03, FIG. 5f), multiple critical enzymes of the glycolysis pathway, such as TPI1 (p=8.31E-01), PFKP (p=7.25E-03) and PGK (p=1.71E-02) (FIG. 5f), and marker genes of energy metabolism, such as AK2 and AK4, indicating a metabolic switch from lipolytic degradation of triglycerides to glycolytic activity. Although a causal link between visceral adipose mass and insulin resistance has been widely observed (Lebovitz and Banerji 2005), the mechanism behind this observation is largely unknown. Together, orthogonal evidence from high-content image-based and RNAseq-based profiling experiments in subcutaneous and visceral AMSCs suggests that individuals with high polygenic risk for HOMA-IR are characterized by a block of lipid degradation in visceral adipocytes.

Polygenic Risk for Lipodystrophy-Like Phenotype Manifests in Cellular Programs that Indicate Reduced Lipid Accumulation Capacity in Subcutaneous Adipocytes

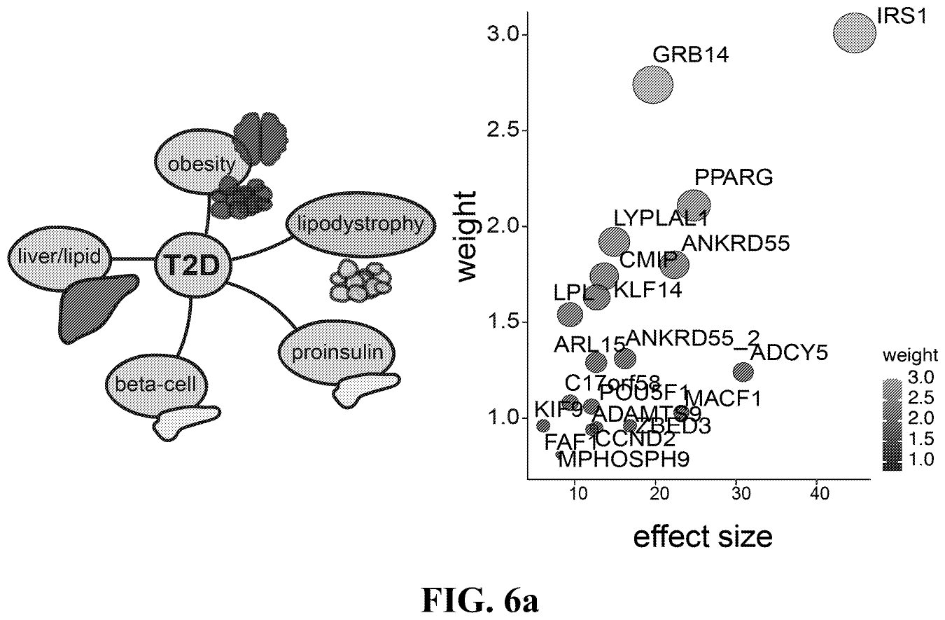

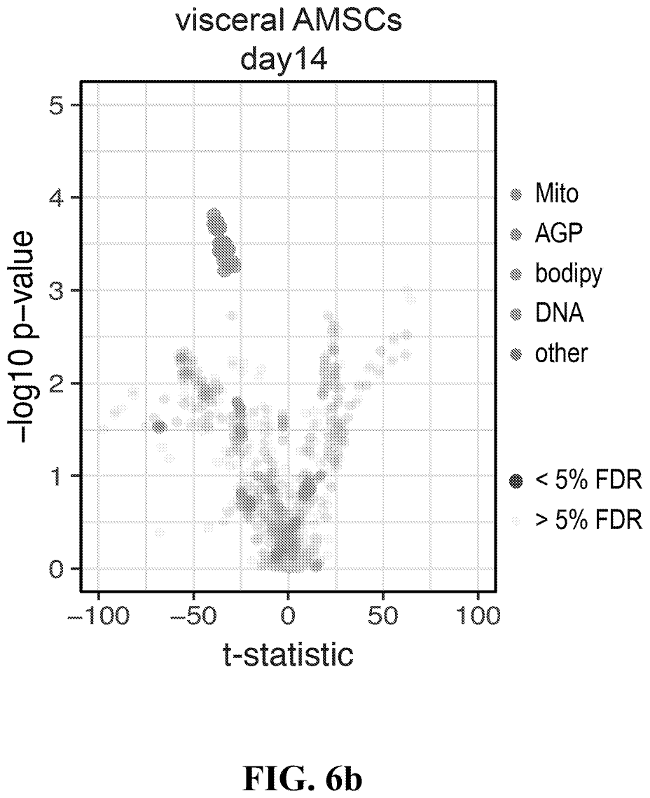

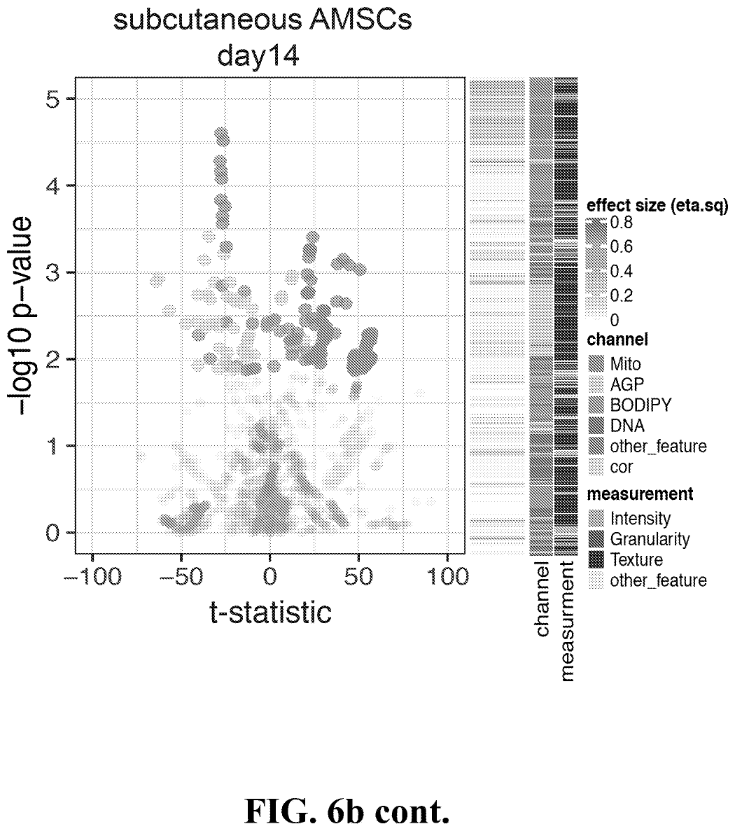

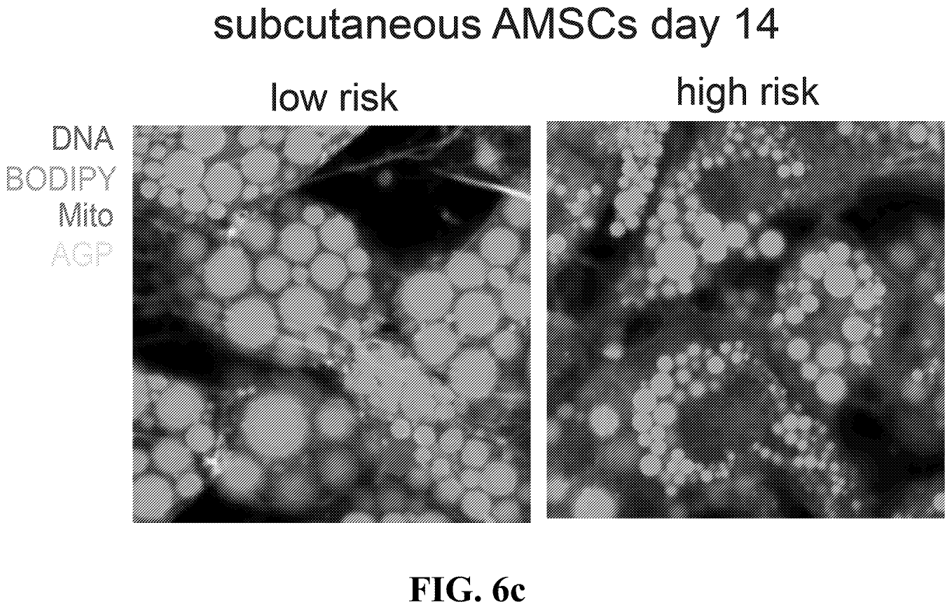

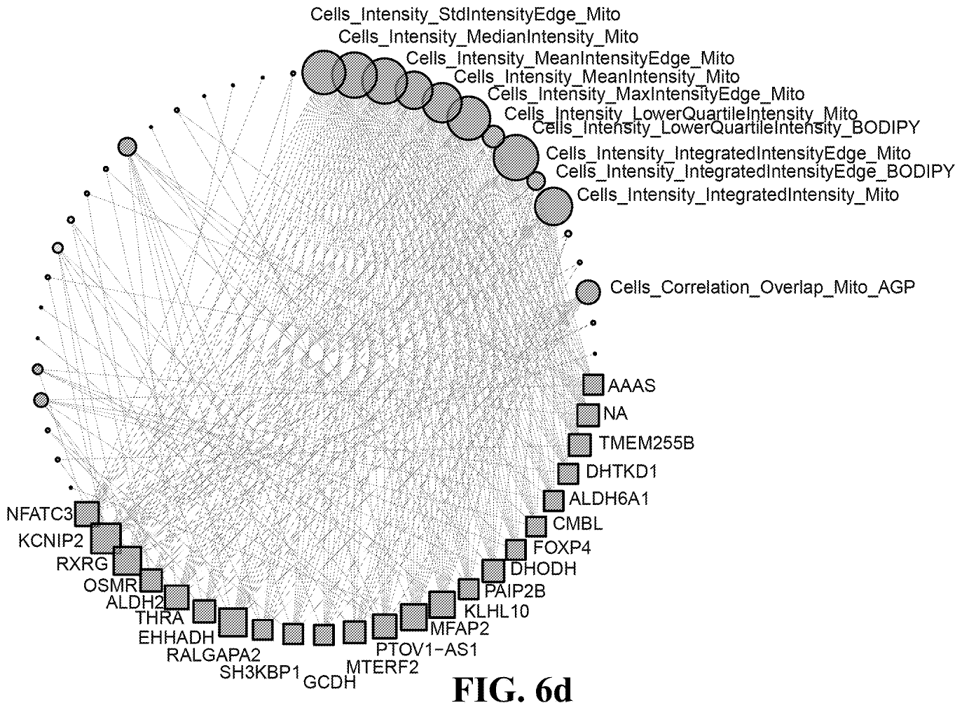

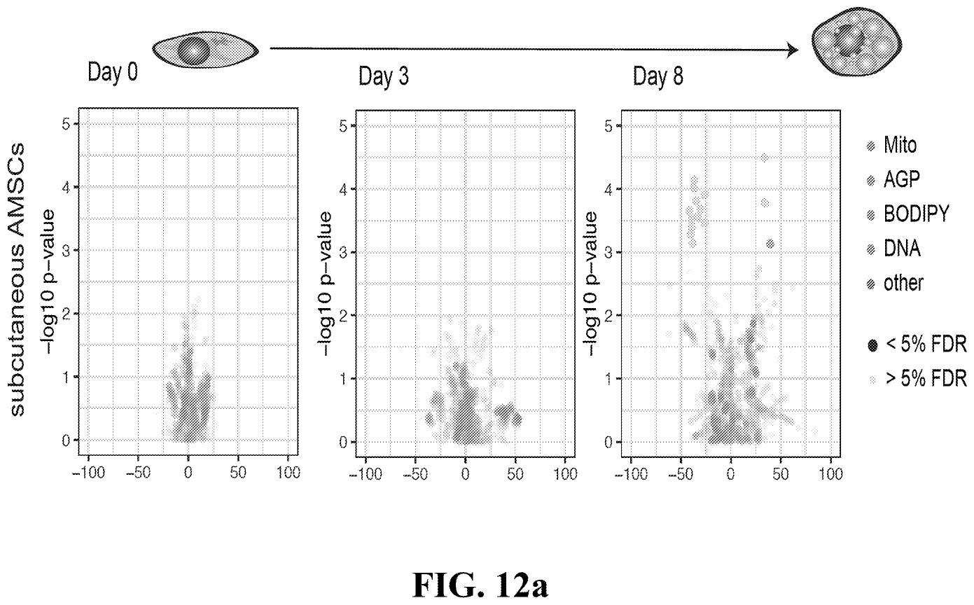

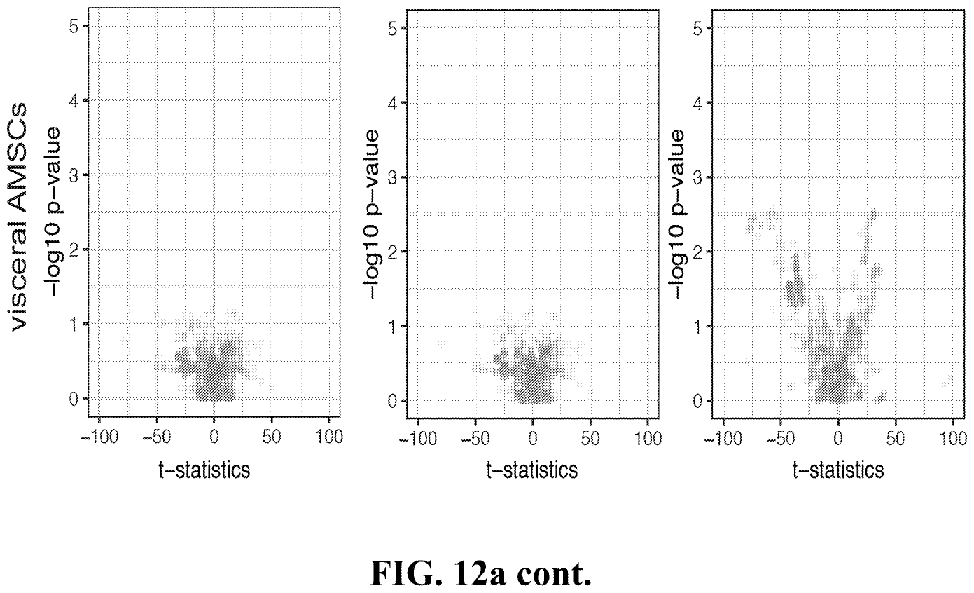

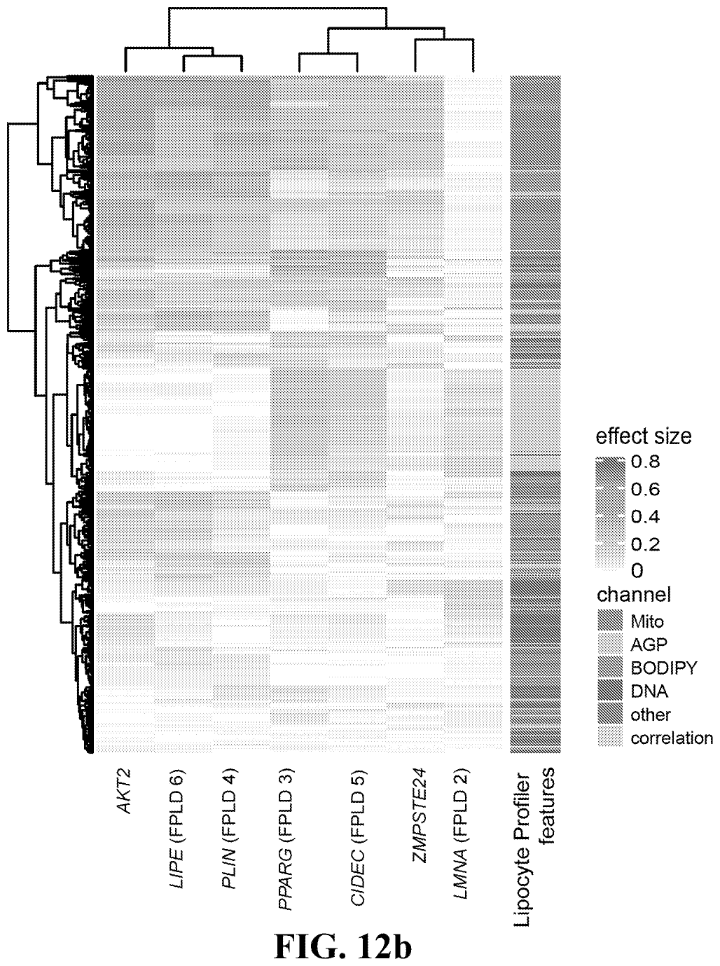

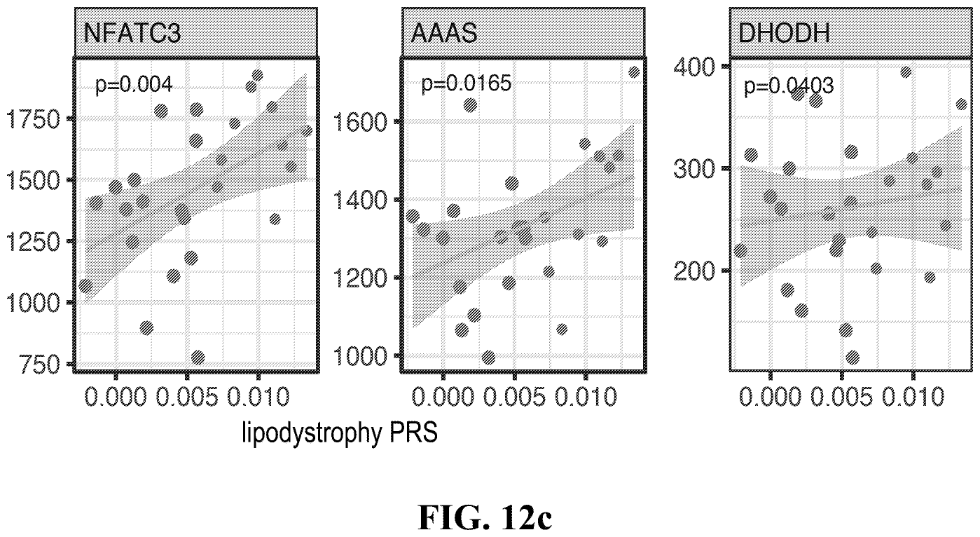

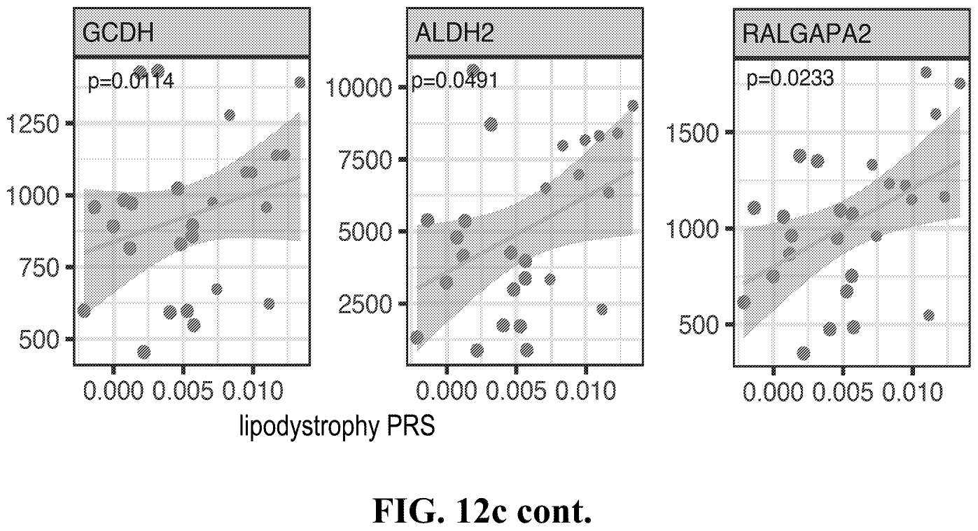

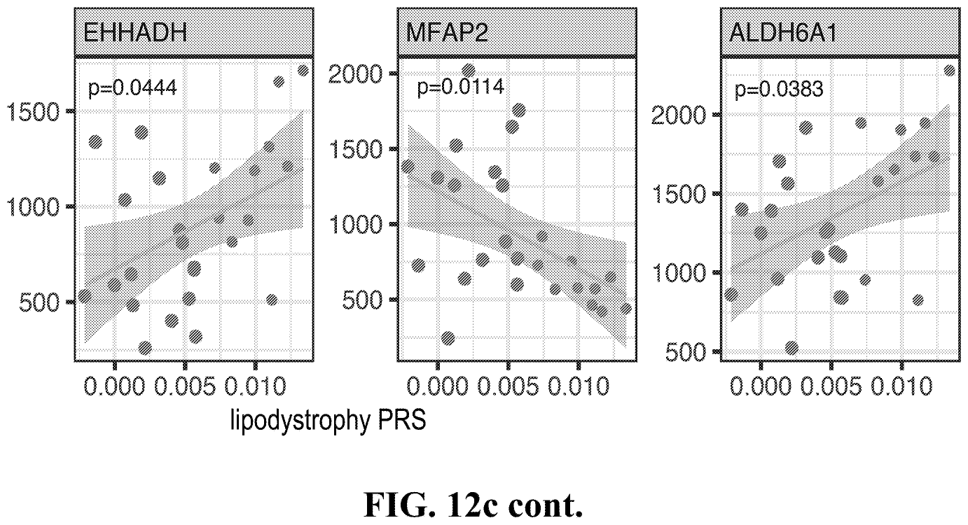

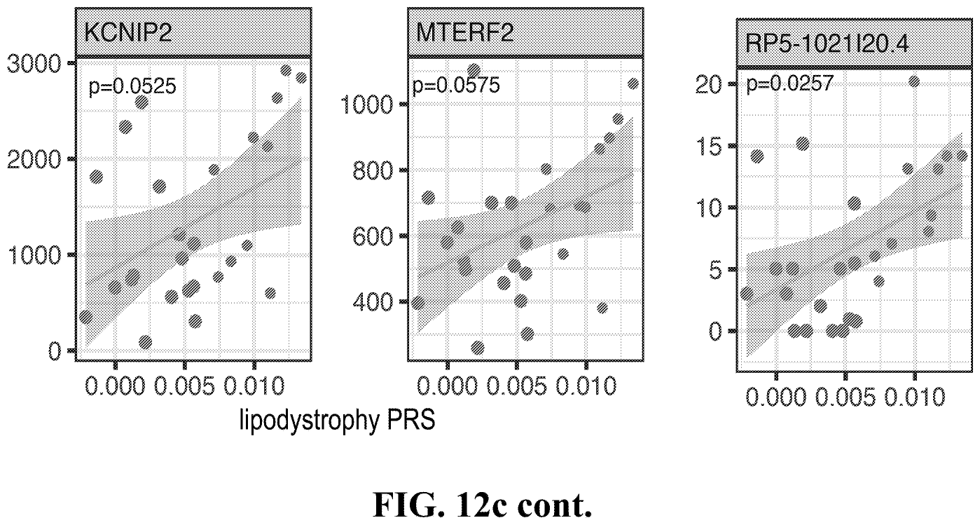

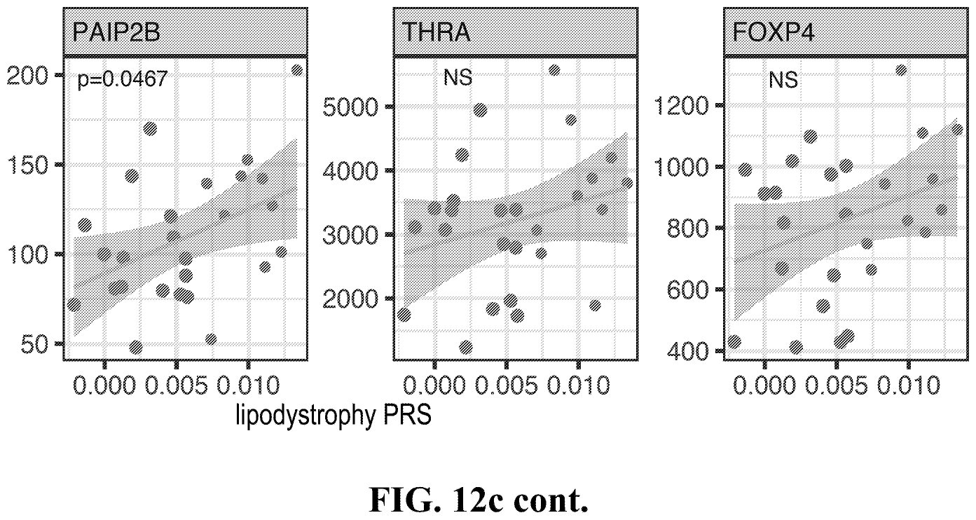

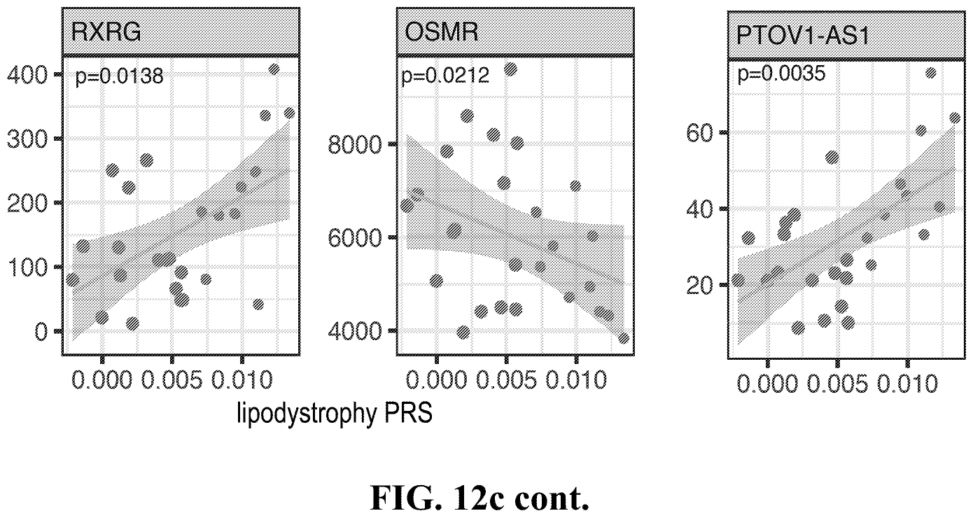

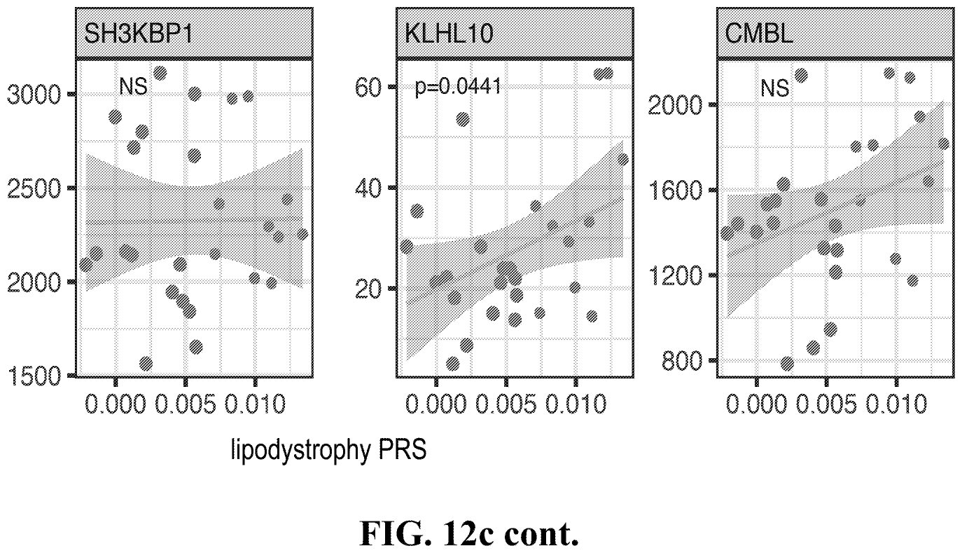

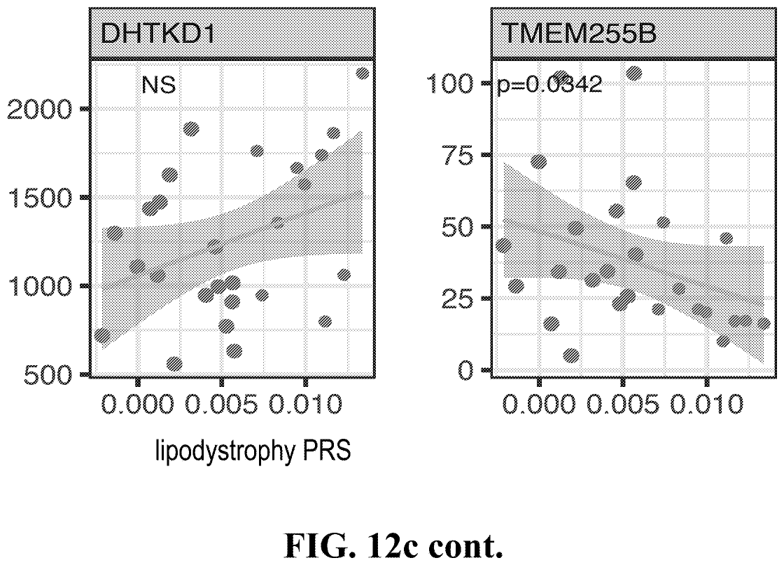

To resolve polygenic effects on adipocyte cellular programs beyond heterogeneous T2D and insulin resistance traits, Applicants used clinically informed process-specific, partitioned PRSs of lipodystrophy (Udler et al. 2018) and correlated those scores with morphological features throughout adipocyte differentiation. Those lipodystrophy PRSs were constructed based on 20 T2D-associated loci with a lipodystrophy-like phenotype (FIG. 6a). Applicants found that polygenic risk of lipodystrophy correlated with predominantly mitochondrial, AGP and BODIPY features in subcutaneous AMSCs at day 14 of differentiation, whereas in mature visceral AMSCs mostly BODIPY features were associated with increased polygenic risk (FIG. 6b, FIG. 12a, Table 8), again highlighting a depot-specific and spatiotemporal-dependent effect of polygenetic risk on morphological profiles captured with LipocyteProfiler. Prototype images of average subcutaneous adipocytes of individuals at the tail ends of lipodystrophy polygenic risk (top and bottom 25th percentile) visibly show that adipocytes from high polygenic risk carriers have increased mitochondrial stain intensity, suggestive of higher mitochondrial activity, accompanied by on average smaller lipid droplets compared to adipocytes from individuals with low polygenic risk (FIG. 6b). Morphological and cellular profiles of marker genes of monogenic familial partial lipodystrophy syndromes like PPARG, LIPE, PLIN1, AKT2, CIDEC, LMNA and ZMPSTE24 show similar morphological signatures to the polygenic lipodystrophy profile with high effect sizes of mitochondrial and AGP features (FIG. 12b), suggesting that polygenic risk and monogenic forms of lipodystrophy converge on similar cellular mechanisms. This notion is particularly interesting in the context of the finding that different monogenic forms of lipodystrophy (independent of the mutation) showed similar consequence on mitochondrial OXPHOS in patient samples (Sleigh et al. 2012).

To identify cellular pathways of lipodystrophy polygenic risk that could underlie the morphological signature in subcutaneous adipocytes, Applicants created a network of genes linked to features identified to be under the control of lipodystrophy polygenic risk. This analysis identified 23 genes that had equal or more than 10 connections to features derived from the polygenic risk lipodystrophy LipocyteProfile (FDR 0.1%, FIG. 6c). 18 of those identified genes were significantly correlated with PRS (FIG. 12c). For example, Applicants found EHHADH, a marker gene of peroxisomal b-oxidation, and NFATC3, which was previously linked to a lipodystrophic phenotype in mice (Hu et al. 2018), to be positively correlated with an increase of polygenic risk (p=0.0444, EHHADH; p=0.004, NFATC3; FIG. 12c). Those results suggest that gene networks identified through morphological signatures recapitulate mechanisms of polygenic risk and LipocyteProfiler can be used to identify molecular mechanisms of disease risk.

### Allele-Specific Effect of the 2p23.3 Lipodystrophy Locus on Mitochondrial Fragmentation and Lipid Accumulation in Visceral Adipocytes

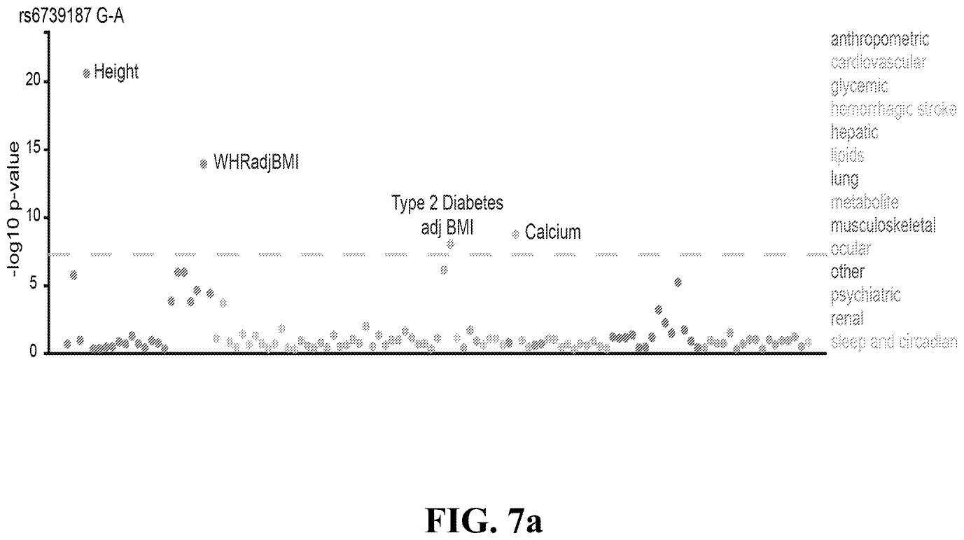

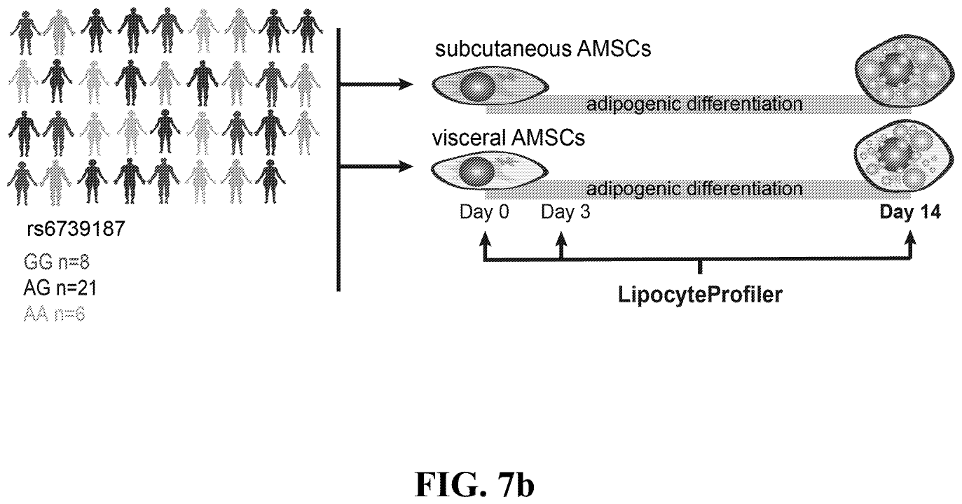

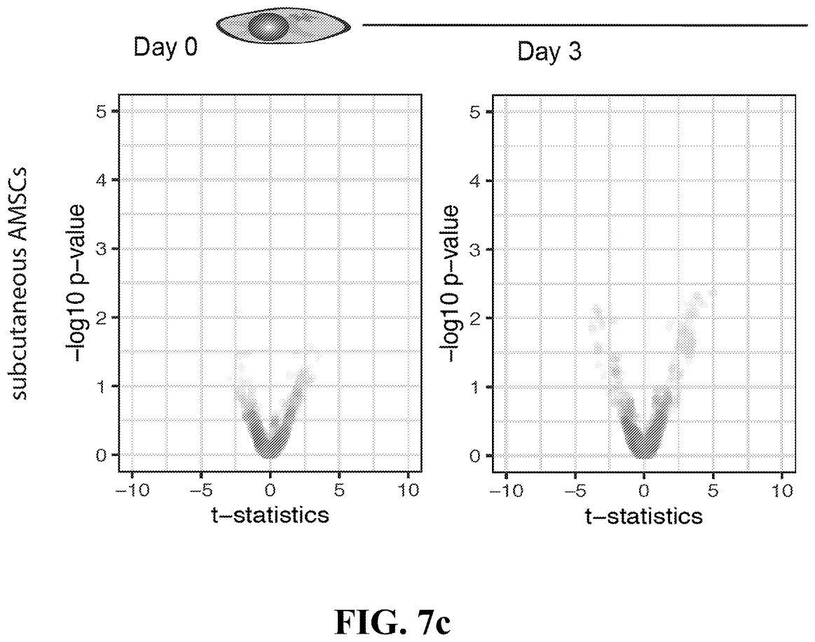

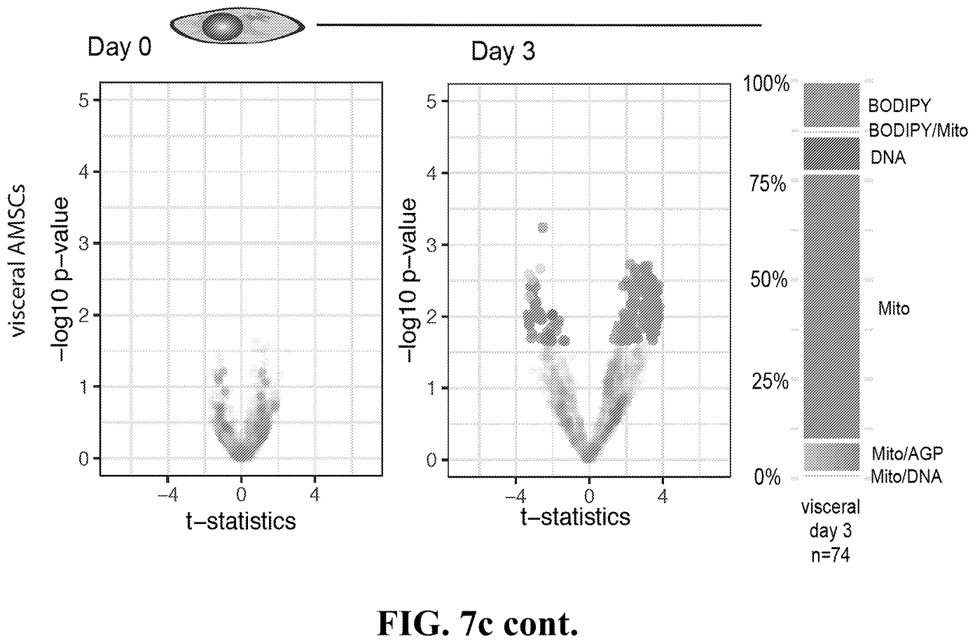

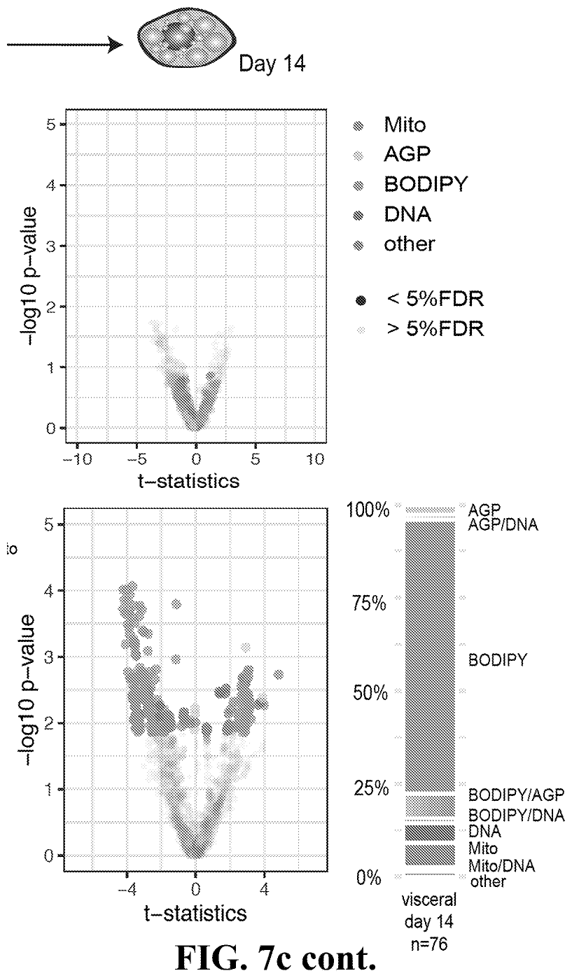

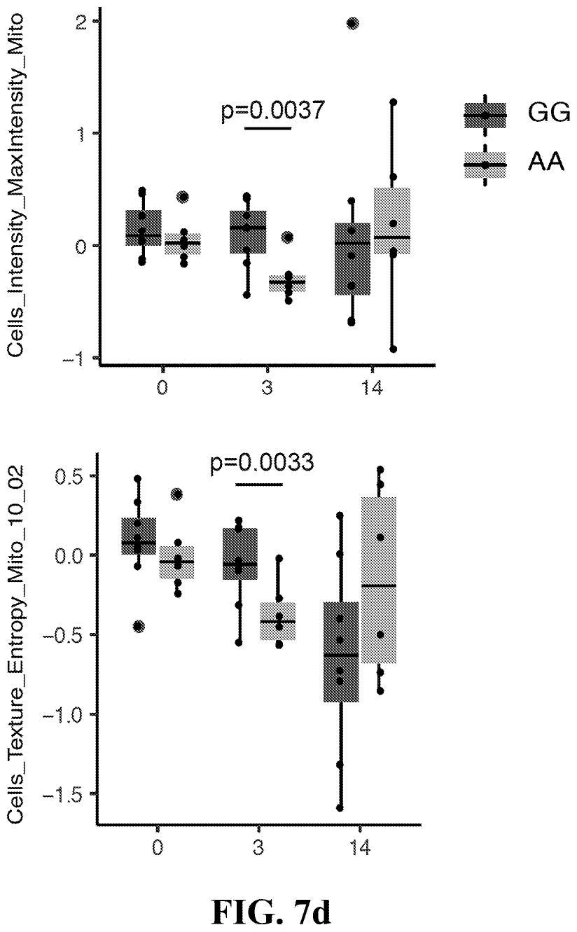

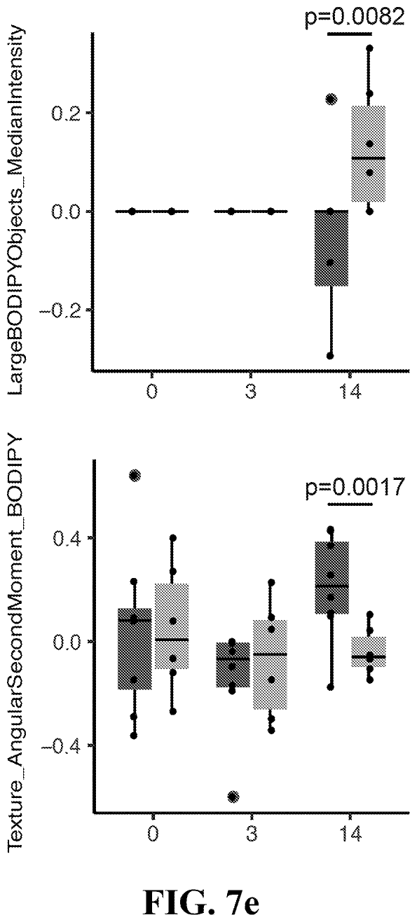

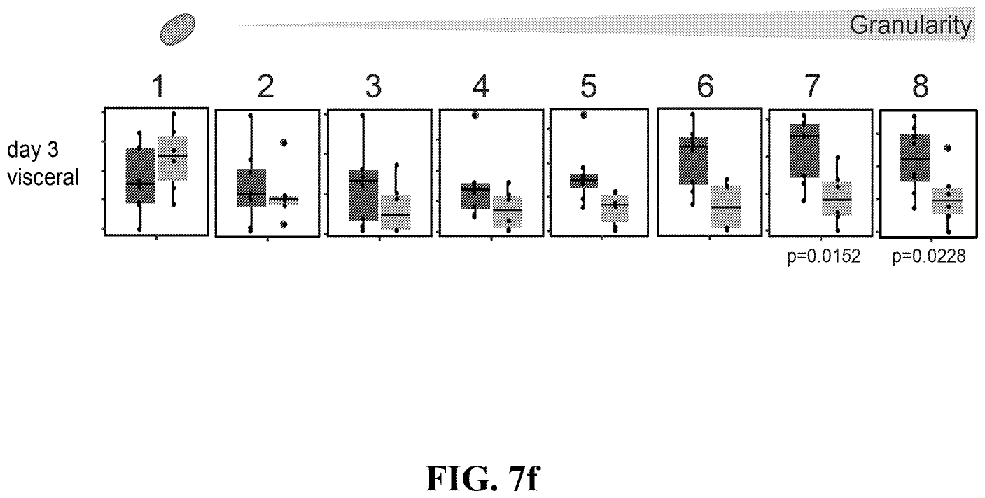

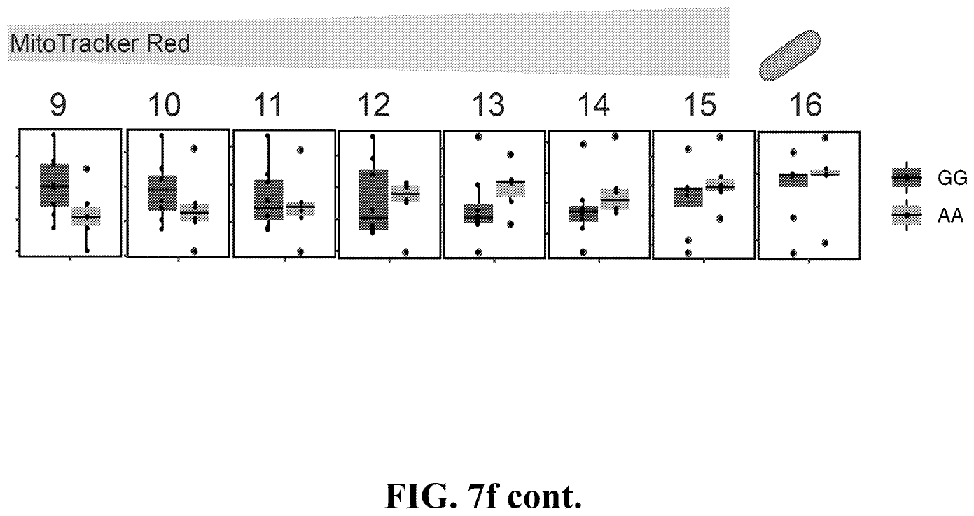

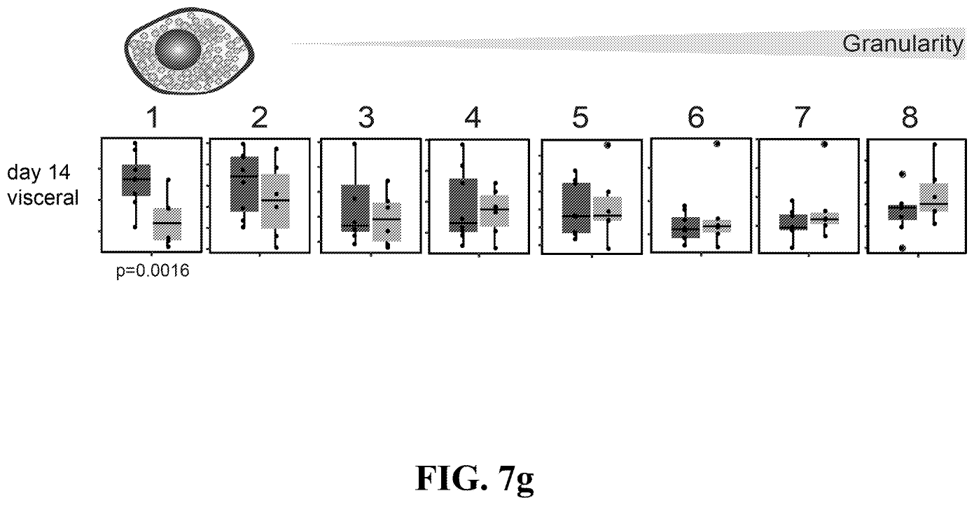

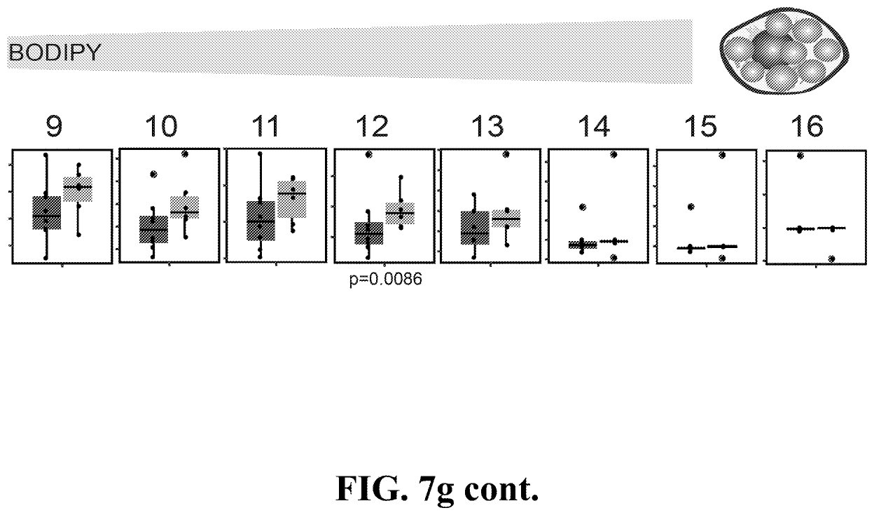

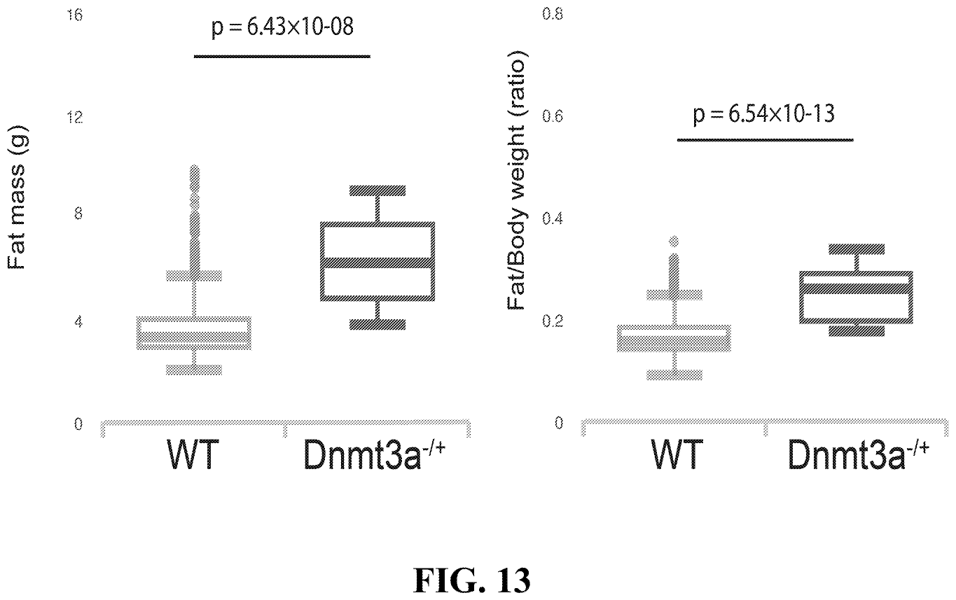

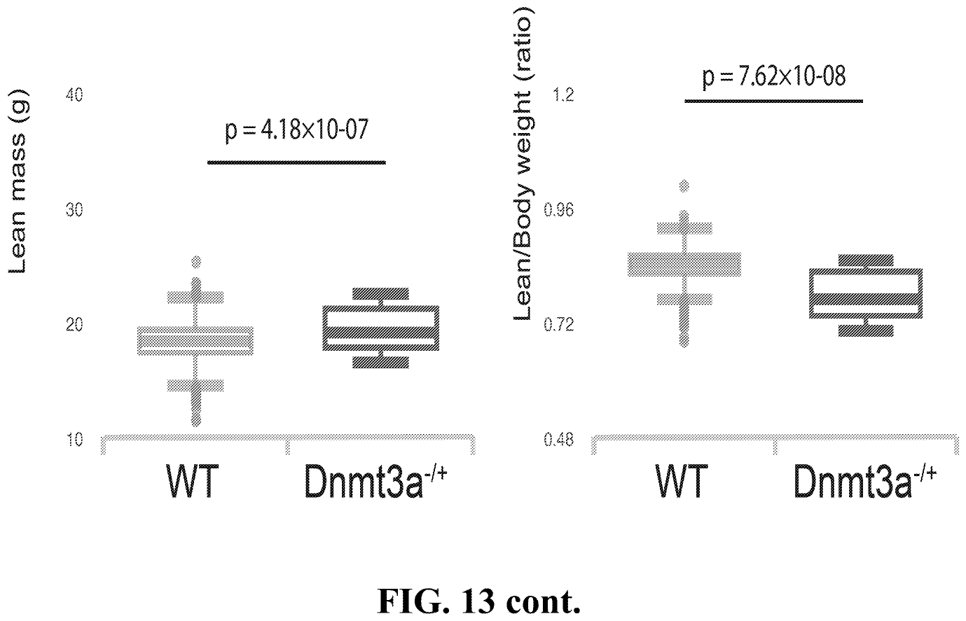

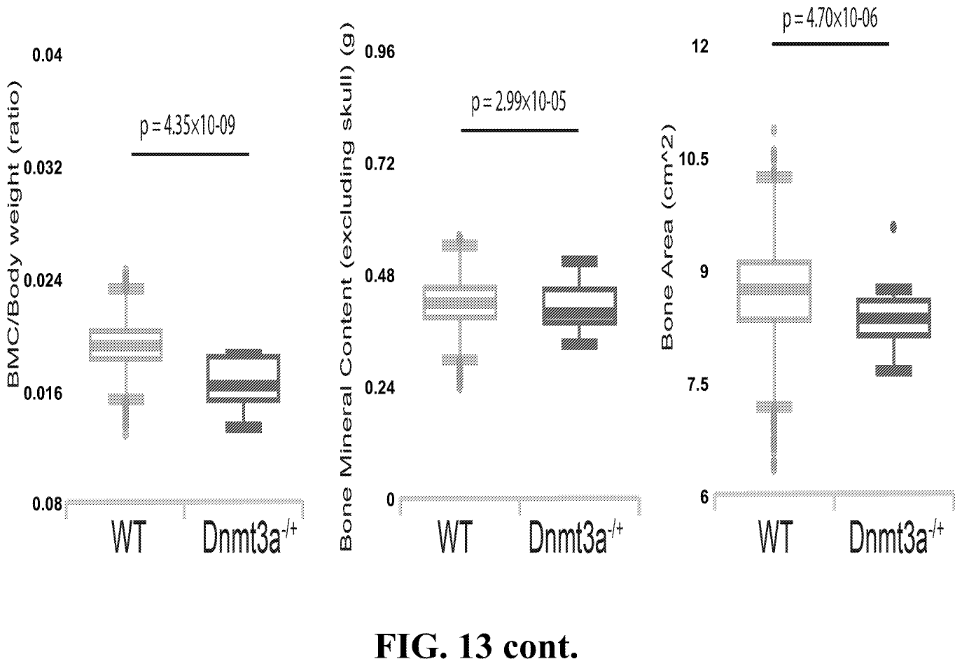

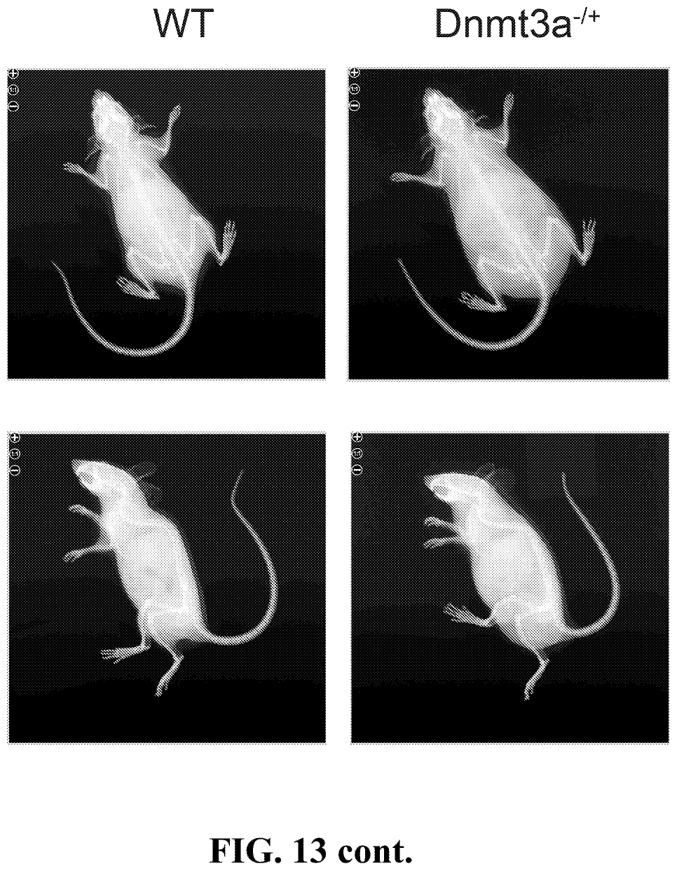

To confirm that Applicants can apply LipocyteProfiler to link an individual genetic risk locus to meaningful cellular profiles in adipocytes Applicants investigated a locus on chromosome 2, location 2p23.3, spanning the DNMT34 gene. The metabolic risk haplotype (minor allele frequency of 0.35 in 1000 Genome Phase 3 combined populations), associated with higher risk for T2D and WHIRadjBMI (FIG. 7a). To map the 2p23.3 metabolic risk locus to cellular functions, Applicants compared LipocyteProfiles of subcutaneous and visceral AMSCs of risk and non-risk haplotype carriers at 3 time points during adipocyte differentiation (before (day 0), early (day 3) and terminal differentiation (day 14)) (FIG. 7b). Applicants observed that in visceral AMSCs 83 and 78 features were significantly different between haplotypes at day 3 and day 14 of differentiation, respectively (FIG. 7c, Table 9), where 70% of differential features at day 3 being mitochondrial, whereas 80% of those features different at day 14 were BODIPY-related. These findings suggest that the 2p23.3 locus is associated with an intermediate phenotype on mitochondrial function during early differentiation that progresses to altered lipid droplet formation in mature visceral adipocytes. At day 3 of differentiation, some of the top-scoring mitochondrial features, mitochondrial Intensity (p=0.0037), Entropy (p=0.0033; FIG. 7d), and mitochondrial granularity features were increased in metabolic risk carriers, suggestive of less tubular and more active mitochondria. At day 14 of differentiation, AMSCs from metabolic risk haplotype carriers showed decreased BODIPY Intensity (FIG. 7e), increased BODIPY AngularSecondMoment and size-specific changes in Granularity, with smaller sized lipid droplets being increased (size 1) and large sized lipid droplets (size 12) being decreased, suggesting a perturbed lipid phenotype of reduced lipid droplet stabilization and/or formation. This profile is associated with increased adipocyte size estimates in visceral adipose tissue as shown in FIG. 3 and suggests that risk haplotype carriers have hypertrophy of visceral WAT. Applicants further note that the findings in adipose are corroborated by organismal perturbation of the candidate effector transcript DNMT3/in mice, where deletion of Dnmt3a results in changes of whole body fat mass (FIG. 13) and protects from high-fat-diet induced insulin resistance, mainly attributed to visceral adipose tissue (You et al. 2017). Together, the data demonstrate that LipocyteProfiler captures complex cellular phenotypes associated with the genetic risk for metabolic diseases and traits, and allows to effectively resolve spatio-temporal context of action. With LipocyteProfiler Applicants generated a resource that enables unbiased mechanistic interrogation of hundreds of metabolic disease loci for which functions are still unknown.

### LipocyteProfiler Discussion

In this study, Applicants present a new imaging framework, LipocyteProfiler, and demonstrate its power in unraveling causal disease mechanisms. Applicants showed that the mechanistic information gained from LipocyteProfiles is not limited to generic cellular organelles but reflects a physiological state of the cell that yields insight into disease-relevant cellular mechanisms. Using LipocyteProfiler, Applicants were able to detect subtle phenotypic differences driven by drug treatment and natural genetic variation at relatively small sample size. This is potentially due to the design of LipocyteProfiler presenting a more granular assay that has high sensitivity for small effect sizes because it assesses cellular phenotypes that present the amelioration of genomic, transcriptional and proteomic states. Applicants showed that polygenic risk for T2D-related traits converge into discrete pathways and mechanisms and demonstrated that LipocyteProfiler determines morphological and cellular signatures underlying differential polygenic risk that were specific to adipocyte depot, trait and developmental time point. Applicants generated a resource and assay that enables unbiased mechanistic interrogation of the hundreds of metabolic disease loci whose function still remains unknown. Applicants showed that LipocyteProfiler could be used to characterize and map underlying mechanisms of donor contribution and drug perturbation to cell behavior. This approach can pave the way for future cellular GWAS linking common genetic variation to phenotypes and can accelerate therapeutic pathway discovery.

### LipocyteProfiler Methods

### Human Primary AMSCs Isolation and Differentiation/Abdominal Laparoscopy Cohort-Munich Obesity BioBank/MOBB

Applicants obtained AMSCs from subcutaneous and visceral adipose tissue from patients undergoing a range of abdominal laparoscopic surgeries (sleeve gastrectomy, fundoplication or appendectomy). The visceral adipose tissue is derived from the proximity of the angle of His and subcutaneous adipose tissue obtained from beneath the skin at the site of surgical incision. Additionally, human liposuction material was obtained. Each participant gave written informed consent before inclusion and the study protocol was approved by the ethics committee of the Technical University of Munich (Study No 5716/13). Isolation of AMSCs was performed as previously described (Hauner et al. 2001). For a subset of donors, purity of AMCSs was assessed as previously described (Raajendiran et al. 2019). Briefly, cells were stained with 0.05 ug CD34, 0.125 ug CD29, 0.375 ug CD31, 0.125 ug CD45 per 250K cells and analyzed on CytoFlex together with negative control samples of corresponding AMCSs.

### Flow Cytometry

Purity of AMCSs was assessed as previously described (Raajendiran et al, 2019). Briefly, cells were stained with 0.05 ug CD34, 0.125 ug CD29, 0.375 ug CD31, 0.125 ug CD45 per 250K cells and analyzed on CytoFlex together with negative control samples of corresponding AMCSs.

### Differentiation of Human AMSCs

For imaging, cells were seeded at 10K cells/well in 96-well plates (Cell Carrier, Perkin Elmer #6005550) and induced 4 days after seeding. For RNAseq, cells were seeded at 40K cells/well in 12-well dishes (Corning). Before Induction cells were cultured in proliferation medium (Basic medium consisting of DMEM-F12 1% Penicillin-Streptomycin, 33 μM Biotin and 17 μM Pantothenate supplemented with 0.13 μM Insulin, 0.01 ug/ml EGF, 0.001 ug/ml FGF, 2.5% FCS). Adipogenic differentiation was induced by changing culture medium to induction medium. (Basic medium supplemented with 0.861 pM Insulin, 1 nM T3, 0.1 μM Cortisol, 0.01 mg/ml Transferrin, 1 μM Rosiglitazone, 25 nM Dexamethasone, 2.5 nM IBMX). On day 3 of adipogenic differentiation culture medium was changed to differentiation medium (Basic medium supplemented with 0.861 pM Insulin, 1 nM T3, 0.1 μM Cortisol, 0.01 mg/ml Transferrin). Medium was changed every 3 days. Visceral-derived AMSCs were differentiated by further adding 2% FBS as well as 0.1 mM oleic and linoleic acid to the induction and differentiation media. For isoproterenol stimulation experiments, 1 μM isoproterenol was added to the differentiation media and cells treated overnight.

### Isolation and Adipocyte Diameter Determination of Floating Mature Adipocytes

Mature adipocyte isolation was carried out as described earlier (Fischer B, Schöttl T, Schempp C, et al. Inverse relationship between body mass index and mitochondrial oxidative phosphorylation capacity in human subcutaneous adipocytes. Am J Physiol Endocrinol Metab. 2015; 309 (4): E380-F387). Immediately after isolation, approximately 50 μl of the adipocyte suspension was pipetted onto a glass slide and the diameter of 100 cells was manually determined under a light microscope.

### Primary Human Hepatocytes Culture

Primary human hepatocytes (PHH) were purchased from BioIVT. Donor lot YNZ was used in this study. PHH were thawed and immediately resuspended in CP media (BioIVT) supplemented with torpedo antibiotic (BioIVT). Cell count and viability were assessed by trypan blue exclusion test prior to plating. Hepatocytes were plated onto collagen-coated Cellcarrier-96 Ultra Microplates (Perkin Elmer) at a density of 50,000 cells per well in CP media supplemented. Four hours after plating, media was replaced with fresh CP media. After 24 h, media was replaced with fresh CP media or CP media containing oleic acid (0.3 mM) or metformin (5 mM). Hepatocytes were incubated for an additional 24 h prior to processing.

### LipocytePainting

Human primary AMSCs and PHH were plated in 96-well CellCarrier plates (Perkinelmer #6005550). AMSCs were differentiated for 14 days and high content imaging was performed at day 0, day 3, day 8 and day 14 of adipogenic differentiation. Primary human hepatocytes were stained after 48 h in culture, and 24h following treatment with oleic acid or metformin. On the respective day of the assay, cell culture media was removed and replaced by 0.5 μM Mitotracker staining solution (1 mM MitoTracker Deep Red stock (Invitrogen #M22426) diluted in culture media) to each well followed by 30 minutes incubation at 37° C.′ protected from light. After 30 min Mitotracker staining solution was removed and cells were washed twice with Dulbecco's Phosphate-Buffered Saline (1×), DPBS (Corning® #21-030-CV) and 2.9 μM BODIPY staining solution (3.8 mM BODIPY 505/515 stock (Thermofisher #D) 3921) diluted in DPBS) was added followed by 15 minutes incubation at 37° C. protected from light. Subsequently, cells were fixed by adding 16% Methanol-free Paraformaldehyde, PFA (Electron Microscopy Sciences #15710-S) directly to the BODIPY staining solution to a final concentration of 3.2% and incubated for 20 minutes at RT protected from light. PFA was removed and cells were washed once with Hank's Balanced Salt Solution (1×), HBSS (Gibco #14025076). To permeabilize cells 0.1% Triton X-100 (Sigma Aldrich #X100) was added and incubated at RT for 10 minutes protected from light. After Permeabilization multi-stain solution (10 units of Alexa Fluor™ 568 Phalloidin (ThermoFisher #A12380), 0.01 mg/ml Hoechst 33342 (Invitrogen #H3570), 0.0015 mg/ml Wheat Germ Agglutinin, Alexa Fluor™ 555 Conjugate (ThermoFisher #W32464), 3 uM SYTO™ 14 Green Fluorescent Nucleic Acid Stain (Invitrogen #/S7576) diluted in HBSS) was added and cells were incubated at RT for 10 minutes protected from light. Finally, staining solution was removed and cells were washed three times with HBSS. Cells were imaged using a Opera Phenix High content screening system. Per well Applicants imaged 25 fields.

### Genotyping and Quality Control of Genotyping Data

DNA was extracted and sent to the Oxford Genotyping Center for genotyping on the Infinium HTS assay on Global Screening Array bead-chips. Genotype QC was done using GenomeStudio and genotypes were converted into PLINK format for downstream analysis. Applicants checked sample missingness but found no sample with missingness >5%. For the remaining sample quality control (QC) steps, Applicants reduced the genotyping data down to a set of high-quality SNPs. These SNPs were: (a) Common (minor allele frequency >10%); (b) Had missingness <0.1%; (c) Independent, pruned at a linkage disequilibrium (r2) threshold of 0.2; (d) Autosomal only; (e) Outside the lactase locus (chr2), the major histocompatibility complex (MHC, chr6), and outside the inversions on chr8 and chr17; (f) In Hardy-Weinberg equilibrium (P>1×10 3).

Using the remaining ˜65,000 SNPs, Applicants checked samples for inbreeding (--het in PLINK), but found no samples with excess homozygosity or heterozygosity (no sample >6 standard deviations from the mean). Applicants also checked for relatedness (--genome in PLINK) and found one pair of samples to be identical; Applicants kept the sample with the higher overall genotyping rate. Finally, Applicants performed PCA using EIGENSTRAT and projected the samples onto data from HapMap3, which includes samples from 11 global populations. Six samples appeared to have some amount of non-European ancestral background, while the majority of samples appeared to be of European descent. Applicants removed no samples at this step, selecting to adjust for principal components in genome-wide testing. However, adjustment for principal components failed to eliminate population stratification, and Applicants therefore restricted to samples of European descent only, defined as samples falling within +/−10 standard deviations of the first and second principal component values of the CEU (Northern and Western European-ancestry samples living in Utah) and TSI (Tuscans in Italy) samples included in the HapMap 3 dataset.4 2, 43 Finally, sex information was received after initial sample QC was complete. As a result, one sample with potentially mismatching sex information (comparing genotypes and phenotype information) was discovered after analyses were complete and therefore remained in the analysis.

### SNP Quality Control

Applicants removed all SNPs with missingness >5% and out of HWE, P<1×10⁻⁶. Applicants also removed monomorphic SNPs. Finally, Applicants set heterozygous haploid sites to missing to enable downstream imputation.

The final cleaned dataset included 190 samples and ˜700,000 SNPs. Applicants note that histology data was not available for all genotyped samples.

### Genotype Imputation

For the genotyped cohorts without imputation data (ENDOX and MOBB) Applicants performed imputation via the Michigan Imputation Server. Applicants aligned SNPs to the positive strand, and then uploaded the data (in VCF format) to the server. Applicants imputed the data with the Haplotype Reference Consortium (HRC) panel, to be consistent with the fatDIVA data which was already imputed with the HRC panel. Applicants selected EAGLE as the phasing tool to phase the data. To impute chromosome X, Applicants followed the server protocol for imputing this chromosome (including using SHAPEIT to perform the phasing step).

### Genetic Risk Scores for Obesity-Related Traits

### Construction of Genetic Risk Scores

Applicants constructed GRSs for BMI, WHIR, and WHIRadjBMI using independent (12<0.05) primary (“index”, associated with each obesity trait P<5×10⁻⁹) SNPs in the combined-sexes analyses in a recent GWAS3 (see data availability). Applicants excluded SNPs with duplicated positions, missingness >0.05, HWE P<1×10⁻⁶, and minor allele frequency <0.05 in the imputed data, after filtering on imputation info >0.3 in the imputed cohorts and restricting the GTEx cohort to those of European ancestry and excluding one individual due to relatedness. For these analyses, the individual in MOBB with potential sex mis-match between genotypic and phenotypic sex was removed. Only SNPs available in all cohorts after quality control was included, resulting in a final set of 530, 259, and 274 SNPs for BMI, WHIR and WHRadjBMI, respectively. The SNPs were aligned so that the effect allele corresponded to the obesity-trait increasing allele. GRSs were then computed for each participant by taking the sum of the participant's obesity-increasing alleles weighted by the SNPs effect estimate, using plink v1.90b3.5 0.

### Statistical Analyses

Applicants then investigated associations with subcutaneous and visceral mean adipocyte area per 1-unit higher obesity GRS, corresponding to a predicted one standard deviation higher obesity trait, using linear regression in R version 3.4.3.5 1 All analyses were performed both with adipocyte area in μm² and in standard deviation units, computed through rank inverse normal transformation of the residuals and adjusting for any covariates at this stage. Applicants adjusted for age, sex, and ten principal components, and with and without adjusting for BMI in the GTEx, MOBB, and fatDIVA cohorts. As Applicants did not have access to data about age and BMI in the all-female ENDOX cohort, Applicants only adjusted for ten principal components in that cohort and with and without adjusting for chip type. Applicants then meta-analyzed the cohorts, assuming a fixed-effects model. In the main meta-analysis model, ENDOX was included using the adjusted for chip type estimates. As a sensitivity analysis, Applicants also reran the meta-analyses using the ENDOX estimates unadjusted for chip type and completely excluding the ENDOX cohort, yielding highly similar results.

### LipocyteProfiling

Quantitation was performed using CellProfiler 3.1.9. Prior to processing, flat field illumination correction was performed using functions generated from the median intensity across each plate. Nuclei were identified using the DAPI stain and then expanded to identify whole cells using the Phalloidin/WGA and BODIPY stains. Regions of cytoplasm were then determined by removing the Nuclei from the Cell segmentations. Speckles of BODIPY staining were enhanced to assist in detection of small and large individual Lipid objects. For each object set measurements were collected representing size, shape, intensity, granularity, texture, colocalization and distance to neighbouring objects. After LipocyteProfiler (LP) feature extraction data was filtered by applying automated and manual quality control steps. First, fields with a total cell count less than 50 cells were removed. Second, every field was assessed visually and fields that were corrupted by experimental induced technical artifacts were removed. Furthermore, blocklisted features (Way, Gregory (2019): Blocklist Features-Cell Profiler. figshare. Dataset. doi.org/10.6084/m9.figshare.10255811.v3), LP-features measurement category Manders, R W C and Costes, that are known to be noisy and generally unreliable were removed. Additionally, LP-features named SmallLipidObjetcs, that measure small objects stained by SYTO14 rather than lipid informative objects, were also removed. After filtering data were normalised per plate using a robust scaling approach (Pedregosa et al. 2011) that subtracts the median from each variable and divides it by the interquartile range. Individual wells were aggregated for downstream analysis by cell depot and day of differentiation.

Subsequent data analyses were performed in R3.6.1 and Matlab using base packages unless noted. To assess batch effects Applicants visualized the data using a Principle component analysis and quantified it using a Kolmogorov-Smirnov test implemented in the “BEclear” R package (Akulenko et al. 2016). Additionally Applicants performed a k-nearest neighbour (knn) supervised machine learning algorithm implemented in the “class” R package (Venables and Ripley 2002) to investigate the accuracy of predicting biological and technical variation. For this analysis the data set, consisting of 3 different cell types (hWAT, hBAT, SGBS) distributed on the 96-well plate, imaged at 4 days of differentiation, was split into equally balanced testing (n=18) and training (n=56) sets. Accuracy of the classification model was predicted based on three different categories cell type, batch and column of the 96-well plate. (github)

For dimensionality reduction visualisation Uniform manifold approximation and projection maps (UMAP) were created using the UMAP R package version 0.2.7.0 (McInnes et al. 2018) (github). To visualise LipocyteProfiles and their effect size ComplexHeatmap Bioconductor package version 2.7.7 (Gu et al. 2016) was used. (github)

To identify patterns of adipocyte differentiation underlying the morphological profiles a sample progression discovery analysis (SPD)) was performed using the algorithm previously described (Qiu et al. 2011). Briefly, the two adipose depots were analyzed separately and features were clustered into modules based on correlation (correlation coefficient 0.6). Minimal spanning trees (MST) were constructed for each module and MSTs of each module are correlated to each other. Modules that support common MST were selected and an overall MST based on features of all selected modules is reconstructed.

Variance component analysis was performed by fitting multivariable linear regression models-yi˜xi+zi+ . . . —where y denotes an LipocyteProfiler feature of individual i and x, 7, etc. independent variables that could confound identification of biological sources of variability of the dataset. Independent variables are experimental batch, adipose depot, passaging before freezing, season and year of AMSCs isolation, sex, age, BMI, T2D status of individual, LipocyteProfiler feature Cells_Neighbors_PercentTouching_Adjacent corresponding to density of cell seeding and identification numbers of individuals. (github)

To test whether there is a difference of morphological profiles on the tail ends of polygenic risk scores (PRS) for T2D, HOMA-IR and WHRadjBMI a multi-way analysis of variance (ANOVA) was performed. Individuals belonging to top 25% and bottom 25% of PRS score distribution are categorized into a categorical variable with 2 levels, top 25% or 25% bottom, according to their PRS percentile. Differences of morphological profiles are predicted using the categorised PRS variable adjusted for sex, age, BMI and batch. For the process-specific lipodystrophy polygenic risk score a linear regression model was fitted adjusted for sex, age, BMI and batch to predict differences of morphological and cellular profiles. To overcome multiple testing burden p-values were corrected using false positive rate (FDR) described in R package “qvalue” (qvalue). Features with FDR <5% were classified to be significantly impacted by the PRS variable. (github)

### Generating of Average Cells

For each group of interest, cells were pooled and divided into 100 clusters via K-Means clustering (scikit-learn). Individual cells were then sampled from the cluster closest to a theoretical point representing the mean of all object measurements, as determined by a euclidean distance matrix.

### RNA-seq

RNA-seq data were processed using FastQC (Krueger and Others 2015) and spliced reads were mapped using STAR (Dobin et al. 2013) followed by counting gene levels using featureCounts (Liao et al. 2014). Next, raw read counts were normalized using DESseq2 R package (Love et al. 2014). For differential expression analysis on the tail ends of polygenic risk scores (PRS) for HOMA-IR a multi-way analysis of variance (ANOVA) was performed on subset of 512 genes (GSEA hallmark gene sets for adipogenesis, fatty acid metabolism and glycolysis). Individuals belonging to top 25% and bottom 25% of PRS score distribution are categorized into a categorical variable with 2 levels, top 25% or 25% bottom, according to their PRS percentile. Differences in transcriptional profiles are predicted using categorised PRS variable adjusted for sex, age, BMI and batch. To overcome multiple testing burden p-values were corrected using false positive rate (FDR) described in R package “qvalue” (Storey J D, Bass A J, Dabney A, Robinson D, 2020). Genes with FDR <10% were classified to be significantly impacted by PRS and were uploaded to Enrichr to analyze them as a gene list against the WikiPathways.

### Gene Expression and LipocyteProfiler Feature Network

A linear regression model was fitted of 2,760 LP-features and global transcriptome RNA-seq data adjusted for sex, age, BMI and batch in subcutaneous AMSCs at day 14 of differentiation. Gene LP features association were declared to be significant when passing FDR cut-off of 0.1% FDR. LP features belonging to Cells category were used for further analysis. Associations between genes and LP features were visualized using “igraph” R package (Csardi et al. 2006) (github). Genes that are connected to top scoring LP features were uploaded to Enrichr to analyse them as a gene list against WikiPathways or BioPlanet. Adipocyte marker genes, SCD, PLIN2, LIPE, INSR, GLUT4 and TIMM22, were chosen to demonstrate morphological profiles matching their known pathways, by identifying LP features that associate with those genes with a global significant level of 5% FDR. (github)

### CRISPR-Cas9 Mediated Knockout of Adipocyte Marker Genes

Applicants generated a hWAT cell-line stably expressing Cas9 as previously described (Shalem et al. 2014). Applicants validated the generated line by assessing Cas9 activity (90%) and adipocyte differentiation capacity using adipocyte marker gene expression and morphological profiling. CRISPR/Cas9 mediated knockdown of PPARG, PGC1A, MFN1 and PLIN1 was performed in pre-adipocytes (5 days before differentiation) using three replicates per guide and two guides per gene (guide sequences targeting PPARG: ATACACAGGTGCAATCAAAG (SEQ ID NO: 42) and CAACTTTGGGATCAGCTCCG (SEQ ID NO: 43); PGC1A: TATTGAACGCACCTTAAGTG (SEQ ID NO: 44) and AGTCCTCACTGGTGGACACG (SEQ ID NO: 45); MFN1: CACCAGGTCATCTCTCAAGA (SEQ ID NO: 46) and TTATATGGCCAATCCCACTA (SEQ ID NO: 47); PLIN1: TCACGGCAGATACTTACCAG (SEQ ID NO: 48) and TCTGCACGGTGTATCGAGAG (SEQ ID NO: 49)) as well as five non-targeted controls (control guide sequences: ATCAGGCCTTGTCCGTGATT (SEQ ID NO: 50), TACGTCATTAAGAGTTCAAC (SEQ ID NO: 51), GACAGTGAAATTAGCTCCCA (SEQ ID NO: 52), GATTCATACTAAACACTCTA (SEQ ID NO: 53), CCTAGTTCATAAGCTACGCC (SEQ ID NO: 54)) in an 96-well arrayed format. Guide on-target efficiency was assessed using Next-generation sequencing followed by CRISPResso analysis (Pinello et al. 2016). AMSCs were stained using LipocytePainting (see above) on day 14 of differentiation. After feature extraction and QC steps (see also LipocyteProfiling), Applicants removed samples where guide cutting efficiency was <10% or where discrepancy between the two guides was equal or above 10%.

### Quality Control

Genotyping of all samples was performed in two separate batches using the Infinium HTS assay on Global Screening Array bead-chips. Since the two sets of samples were genotyped with different versions of the beadchips and in different batches, Applicants Qced, imputed, and generated the genome-wide polygenic scores separately and combined the results afterwards.

A 3-step quality control protocol was applied using PLINK (Purcell et al. 2007; Chang et al. 2015), and included 2 stages of SNP removal and an intermediate stage of sample exclusion.

The exclusion criteria for genetic markers consisted of: proportion of missingness ≥0.05, HWE p≤1×10⁻²⁰ for all the cohort, and MAF <0.001. This protocol for genetic markers was performed twice, before and after sample exclusion.

For the individuals, Applicants considered the following exclusion criteria: gender discordance, subject relatedness (pairs with PI-HAT ≥0.125 from which Applicants removed the individual with the highest proportion of missingness), sample call rates ≥0.02 and population structure showing more than 4 standard deviations within the distribution of the study population according to the first seven principal components. After QC, 35 subjects remained for the analysis for which Applicants had matched LipocyteProfiler imaging data.

Genotypes were phased with SHAPEIT2 (Delaneau et al. 2013), and then performed genotype imputation with the Michigan Imputation server, using Haplotype Reference Consortium (HRC) (Consortium and the Haplotype Reference . . . ) as reference panel. Applicants excluded variants with an info imputation r-squared <0.5 and a MAF <0.005.

Genome-wide polygenic scores were computed using PRS-CS (Ge et al. 2019) and using the “auto” parameter to specify the phi shrinkage parameter. Applicants computed the PRS-CS polygenic scores for the following traits: T2D (Mahajan et al. 2018), BMI, waist-to-hip ratio adjusted and unadjusted by BMI, and stratified by sex and combined (Pulit et al. 2019). Genome-wide PRS for HOMA-IR were computed with LdPred (Vilhjálmsson et al. 2015) using summary statistics from Dupuis et al (Dupuis et al. 2010).

Process-specific PRSs were constructed based on five clusters defined in Udler et al. (Udler et al. 2018) by selecting the SNPs that had weight larger than 0.75 for each of a given cluster. Applicants used the effect sizes described in Mahajan et al as weight for the polygenic scores (Mahajan et al. 2018).

All PRSs were tested for association with T2D and with BMI using the 30,240 MGB Biobank samples from European Ancestry defined based on self-reported and principal components.

### MGB Biobank Cohort

The MGB Biobank (Karlson et al. 2016) maintains blood and DNA samples from more than 60,000 consented patients seen at Partners HealthCare hospitals, including Massachusetts General Hospital, Brigham and Women's Hospital, McLean Hospital, and Spaulding Rehabilitation Hospital, all in the USA. Patients are recruited in the context of clinical care appointments at more than 40 sites, clinics and also electronically through the patient portal at Partners HealthCare. Biobank subjects provide consent for the use of their samples and data in broad-based research. The Partners Biobank works closely with the Partners Research Patient Data Registry (RPDR), the Partners' enterprise scale data repository designed to foster investigator access to a wide variety of phenotypic data on more than 4 million Partners HealthCare patients. Approval for analysis of Biobank data was obtained by Partners IRB, study 2016P001018.

Type 2 diabetes status was defined based on “curated phenotypes” developed by the Biobank Portal team using both structured and unstructured electronic medical record (EMR) data and clinical, computational and statistical methods. Natural Language Processing (NLP) was used to extract data from narrative text. Chart reviews by disease experts helped identify features and variables associated with particular phenotypes and were also used to validate results of the algorithms. The process produced robust phenotype algorithms that were evaluated using metrics such as sensitivity, the proportion of true positives correctly identified as such, and positive predictive value (PPV), the proportion of individuals classified as cases by the algorithm (Yu et al. 2015).
a. Control selection criteria.
   1. Individuals determined by the “curated disease” algorithm employed above to have no history of type 2 diabetes with NPV of 99%.
   2. Individuals at least age 55.
   3. Individuals with HbA1c less than 5.7
b. Case selection criteria.
   1. Individuals determined by the “curated disease” algorithm employed above to have type 2 diabetes with PPV of 99%
   2. Individuals at least age 30 given the higher rate of false positive diagnoses in younger individuals.
      Genomic data for 30,240 participants was generated with the Illumina Multi-Ethnic Genotyping Array, which covers more than 1.7 million markers, including content from over 36,000 individuals, and is enriched for exome content with >400,000 markers missense, nonsense, indels, and synonymous variants.

A 3-step quality control protocol was applied using PLINK (Purcell et al. 2007; Chang et al. 2015), and included 2 stages of SNP removal and an intermediate stage of sample exclusion.

The exclusion criteria for genetic markers consisted of: proportion of missingness ≥0.05, HWE p≤1×10⁻²⁰ for all the cohort, and MAF <0.001. This protocol for genetic markers was performed twice, before and after sample exclusion.

For the individuals, Applicants considered the following exclusion criteria: gender discordance, subject relatedness (pairs with PI-HAT ≥0.125 from which Applicants removed the individual with the highest proportion of missingness), sample call rates ≥0.02 and population structure showing more than 4 standard deviations within the distribution of the study population according to the first seven principal components.

Genotypes were phased with SHAPEIT2 (Delaneau et al. 2013), and then performed genotype imputation with the Michigan Imputation server, using Haplotype Reference Consortium (HRC) as reference panel. Applicants excluded variants with an info imputation r-squared <0.5 and a MAF <0.005.

### Human Primary AMSCs Isolation and Differentiation

Human liposuction material used for isolation of preadipocytes was obtained from a collaborating private plastic surgery clinic Medaesthetic Privatklinik Hoffmann & Hoffmann in Munich, Germany. Harvested subcutaneous liposuction material was filled into sterile 1 L laboratory bottles and immediately transported to the laboratory in a secure transportation box. The fat was aliquoted into sterile straight-sided wide-mouth jars, excluding the transfer of liposuction fluid. The fat was stored in cold Adipocyte Basal medium (AC-BM) at a 1:1 ratio of fat to medium and stored at 4° C. to be processed the following day. Additionally, small quantities of the original liposuction material would be aliquoted into T-25 flasks at a 1:1 ratio of fat to medium as controls to check for contamination. These control flasks were stored in the 37° C. incubator and were not processed. Krebs-Ringer Phosphate (KRP) buffer was prepared containing 200 U/ml of collagenase and 4% heat shock fraction BSA and sterilized by filtration using a Bottle Top Filter 0.22 μm. When the fat reached RT, 12.5 ml of liposuction material was aliquoted into sterile 50-ml tubes with plug seal caps. The tubes were filled to 47.5 ml with warm KRP-BSA-collagenase buffer and the caps were securely tightened and wrapped in Parafilm to avoid leakage. The tubes were incubated in a shaking water bath for 30 minutes at 37° C. with strong shaking. After 30 minutes, the oil on top was discarded and the supernatant was initially filtered through a nylon mesh. The supernatant of all tubes was combined after filtration and centrifuged at 200×g for 10 minutes. The supernatant was discarded and each pellet was resuspended with 3 ml of erythrocyte lysis buffer, then all the pellets were pulled in one tube and incubated for 10 minutes at RT. The cell suspension was filtered through a 250 μm Filter and then through 150 μm Filter, followed by centrifugation at 200 g for 10 minutes. The supernatant was discarded and the pellet containing preadipocytes was resuspended in an appropriate amount of DMEM/F12 with 1% P/S and 10% FCS and seeded in T75 cell culture flasks and stored in the incubator (37° C., 5% CO2). The next day the medium was changed to PAC-PM. Once preadipocytes reached 100% confluency in T25 or T75 flasks they were split into 6-well plates at a seeding density on 250,000 cells per plate in PAC-PM. Once they reached 100% confluency, PAC-IM was prepared fresh and added to the preadipocytes to induce differentiation. On day 3 after induction, the medium was changed to PAC-DM and replaced twice a week.

### Isolation of Subcutaneous and Visceral AMSCs

Subcutaneous adipose tissue was sampled from the abdominal area at the site of incision and visceral adipose tissue from the angle of his from patients undergoing elective abdominal laparoscopic surgery. Each patient gave written informed consent prior to inclusion and the study protocol was approved by the ethics committee of the Technical University of Munich (Study nr. 5716/13). Connective tissue and blood vessels were dissected and one gram of minced adipose tissue was digested with 5 ml of Krebs-ringer phosphate buffer containing 200 U/ml of collagenase (SERVA, Heidelberg, Germany). Digestion was carried out at 37° C. for 60 minutes in a shaking water bath. Afterwards the suspension was centrifuged at 200 g for 10 minutes and the supernatant was discarded. The pellet containing the SVF was resuspended in DMEM/F12 (Gibco, Thermo Fisher Scientific, Darmstadt) containing 10% FCS (F7524, Sigma-Aldrich, Taufkirchen, Germany) and 1% penicillin-streptomycin (P/S; PAA Laboratories, Linz, Austria). After filtering the cell suspension through a 70 μm cell strainer the cells were plated, washed with PBS on the next day and medium was changed to proliferation medium. Proliferation and differentiation of isolated preadipocytes was carried out as described earlier [DOI[JH1]:10.1056/NEJMoa1502214]

### Iso Liposuction

Human primary AMSCs were isolated from liposuction material. Each patient gave written informed consent prior to inclusion and the study protocol was approved by the ethics committee of the Technical University of Munich (study nr. 5716/13). The liposuction material was immediately transported to the laboratory and stored with an equal amount of DMEM-F12 (Gibco, Thermo Fisher Scientific, Darmstadt) containing 1% penicillin-streptomycin (P/S; PAA Laboratories, Linz, Austria) over night at 4° C. On the next day the samples were digested in a 1:4 ration with Krebs-Ringer Phosphate (KRP) buffer containing 200 U/ml collagenase (SERVA, Heidelberg, Germany) at 37° C. in a shaking water bath for 60 minutes. After digestion the adipocyte/oil containing layer was removed and the remaining liquid containing the SVF was filtered through a 2000 μm nylon mesh. The SVF was pelleted through centrifugation for 10 minutes at 200 g. The supernatant was discarded and the pellet was resuspended in 37 Â° C. warm erythrocyte lysis buffer (155 mM NH₄Cl, 5.7 mM K2HPO₄, 0.1 mM EDTA dihydrate) and incubated at room temperature for 10 minutes. The cell suspension was filtered through a 250 Î ¼ m Filter and then through a 150 μm Filter, followed by centrifugation at 200 g for 10 minutes. The supernatant was discarded and the pellet containing AMSCs was resuspended in DMEM/F12 containing 1% P/S and 10% FCS (Sigma, F7524). Cells were seeded and washed with PBS on the next day before switching to proliferation medium. Proliferation and differentiation was carried out as described earlier [DOI[JH3]: 10.1056/NEJMoa1502214]

### Example 2—COBLL1

The 2q24.3 MONW Risk Locus Overlaps with Enhancer Signatures in Adipocyte Progenitors

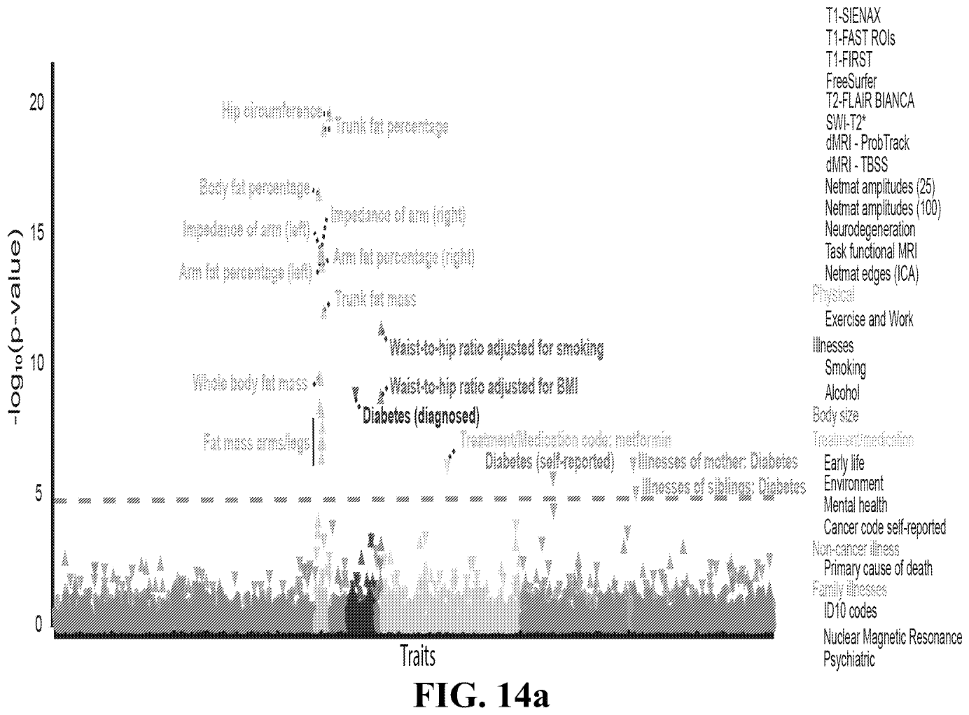

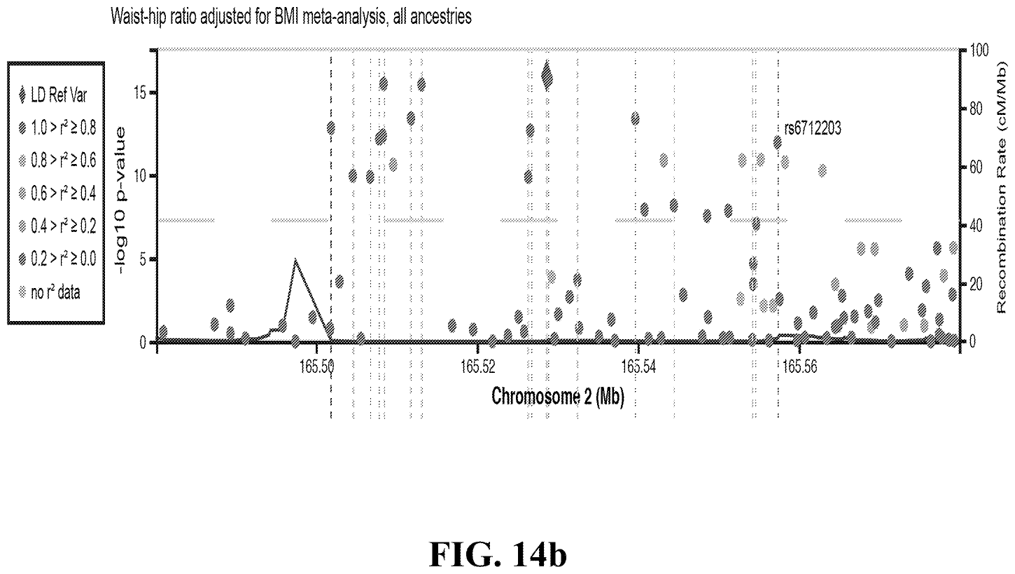

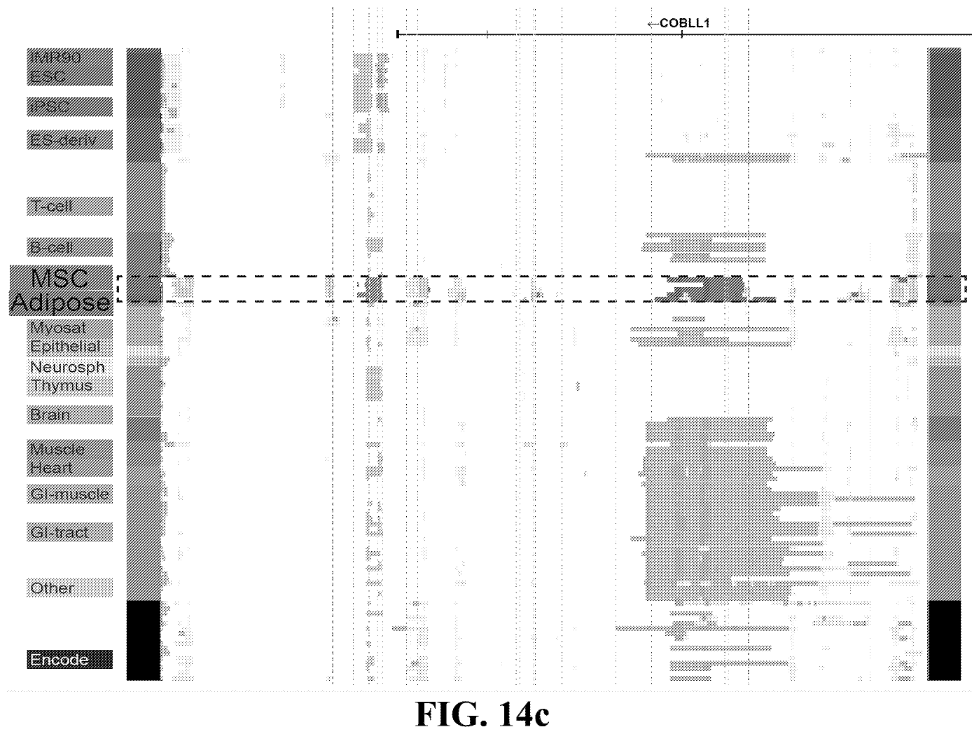

To identify diseases and traits associated with the 2q24.3 locus, Applicants visualized large-scale phenome-wide associations from the UK Biobank (UKBB) (Gagliano et al., 2020). Jointly analyzing phenotypes across the UKBB Applicants observed that the 2q24.3 locus associated with increased T2D risk as well as a series of body fat-related traits (FIG. 14a), including increased WHIRadjBMI, but decreased trunk fat percentage, arm fat percentage, hip circumference, and whole body fat mass, suggesting a complex pleiotropic risk locus consistent with a MONW association signature, i.e. a lean, metabolically unhealthy phenotype.

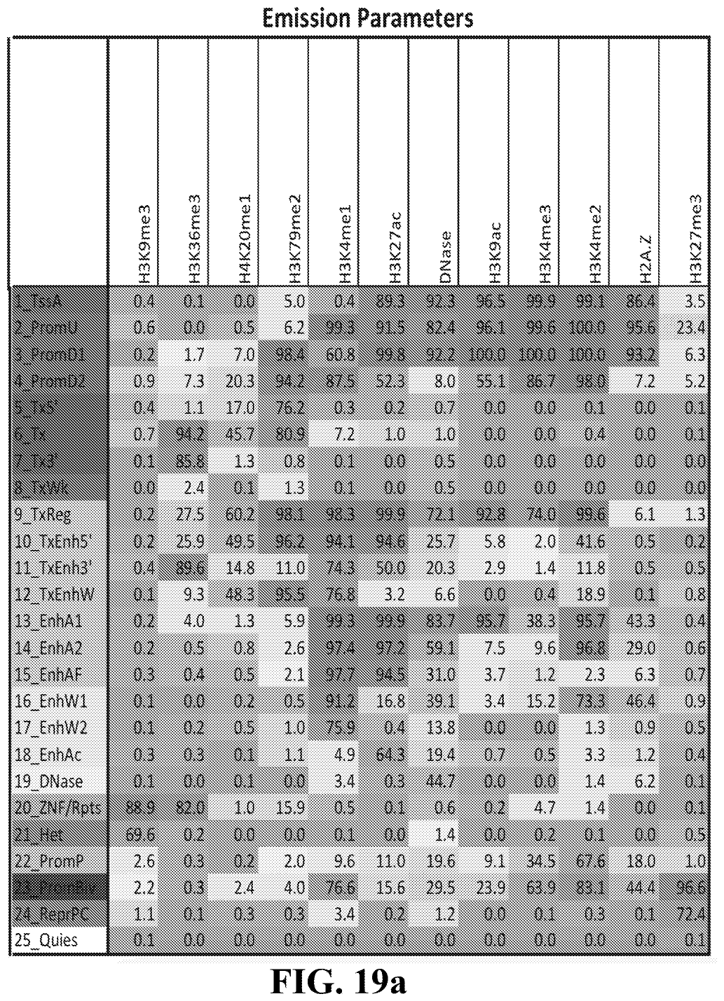

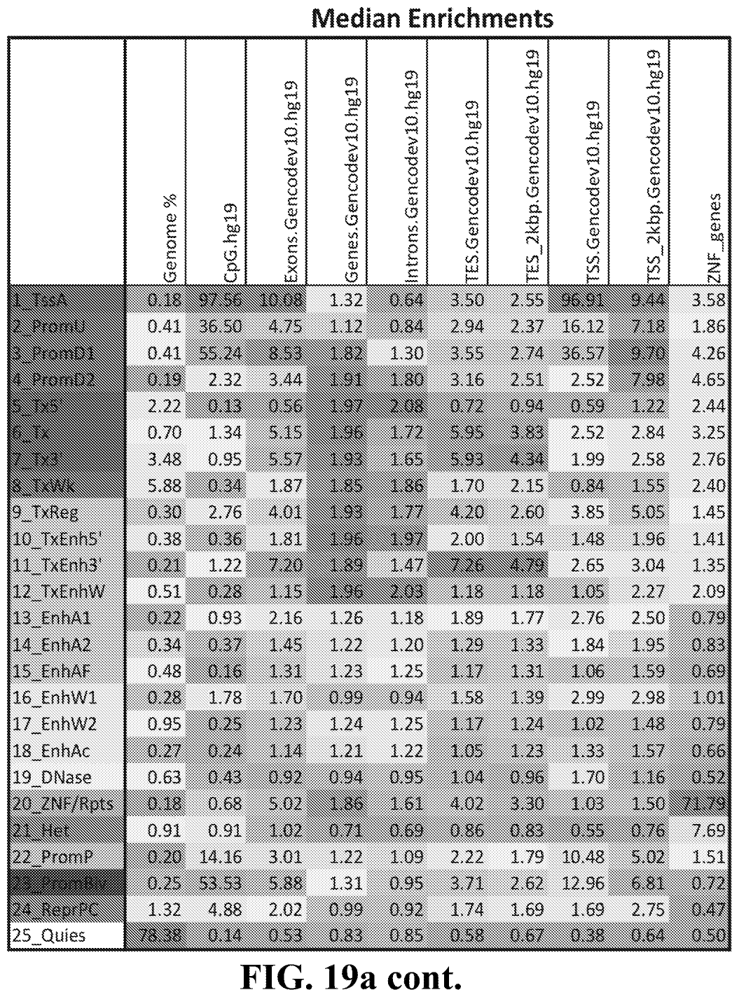

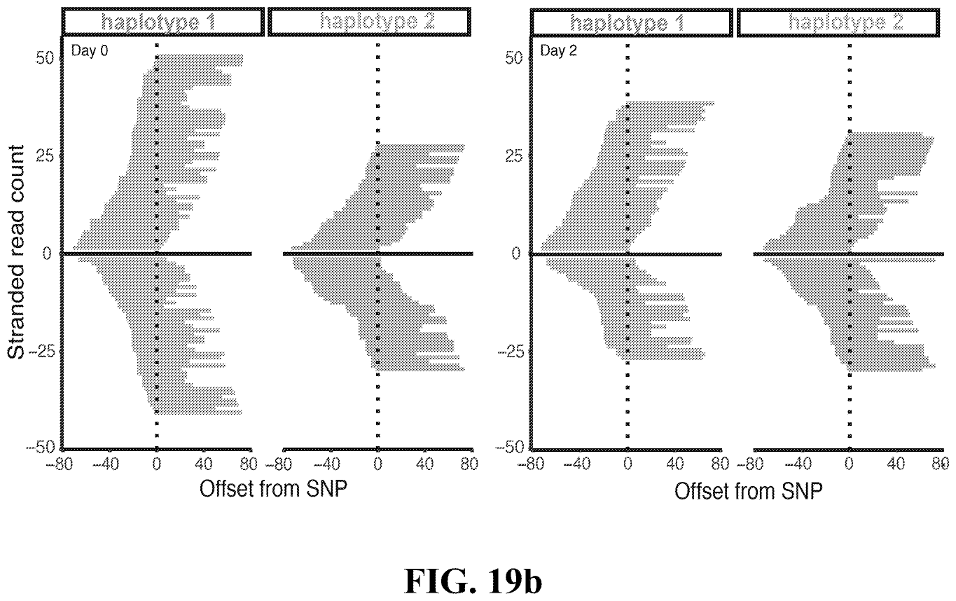

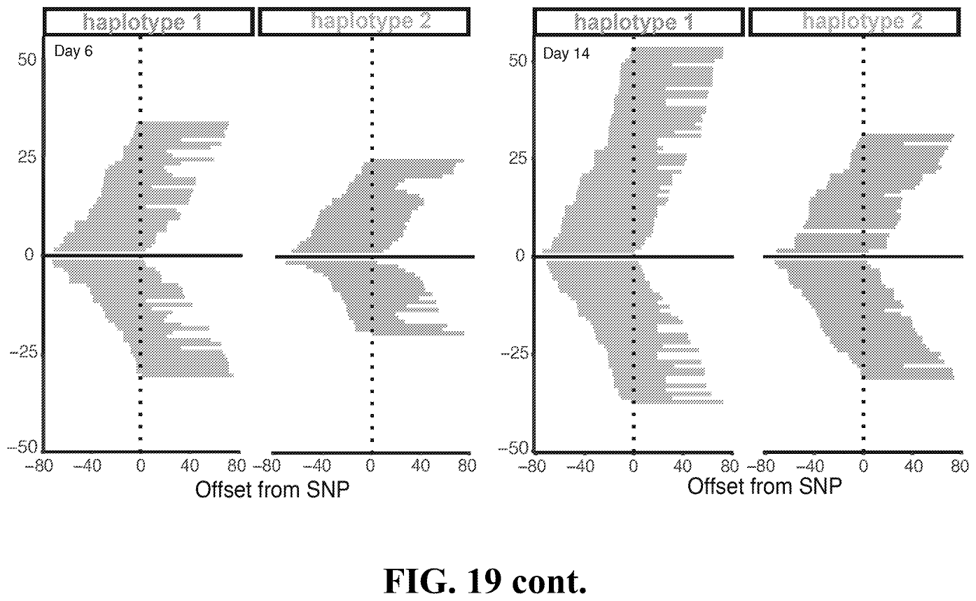

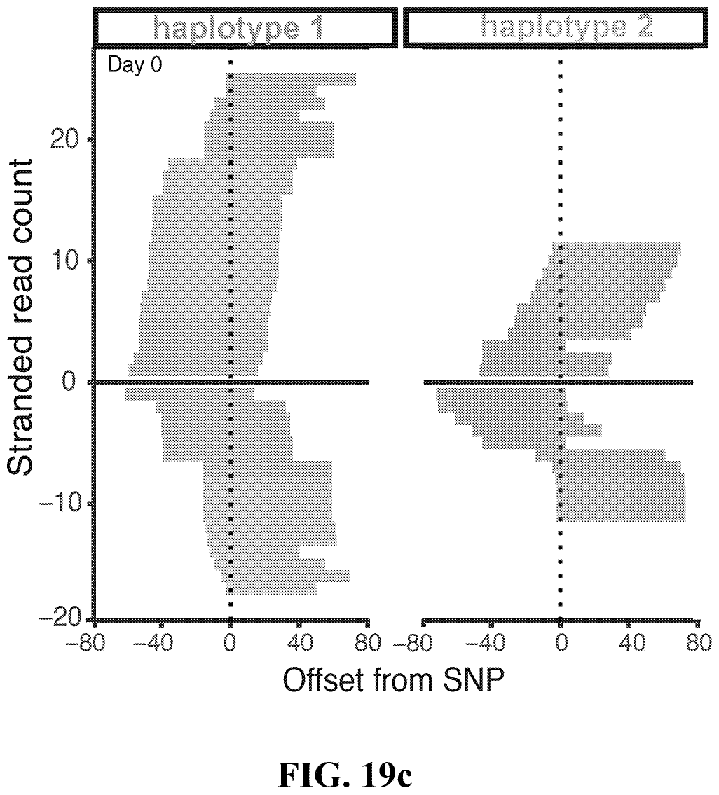

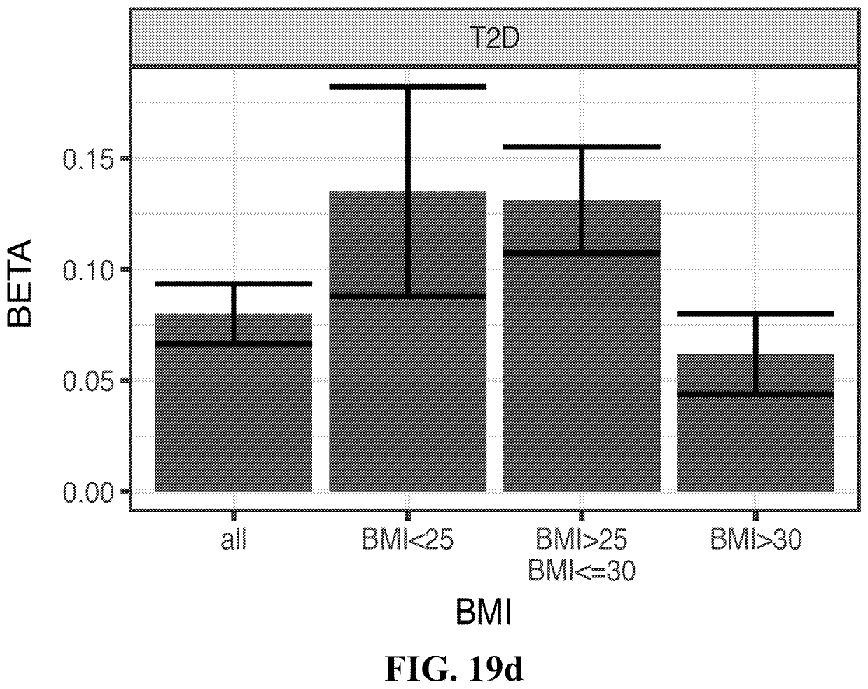

The 2q24.3 locus encompasses 55 kilobases, spanning from COBLL1 intronic regions to the intergenic region between GRB14 and COBLL1 (FIG. 14b). The MNOW locus harbors 19 non-coding variants in high linkage disequilibrium (LD) (r²>0.8, 1000G Phase 1 EUR). To connect genetic variants at the 2q24.3 locus to relevant cell types and cell states, Applicants examined chromatin state maps across 127 reference epigenomes from the Roadmap Epigenomics and the ENCODE consortium (FIG. 14c, FIG. 19a). Applicants found that the locus is characterized by quiescent chromatin in most cell types and tissues, with the exception of enhancer signatures in mesenchymal stem cells, adipocyte progenitors and adipocytes (FIG. 14c). Several of the 19 non-coding variants map within or in the vicinity of regions with active enhancer chromatin states, suggesting that the 2q24.3 locus acts in adipocytes through gene regulatory mechanisms.

Next, Applicants examined whether the two haplotypes show differences in chromatin structure during adipocyte differentiation. Specifically, Applicants performed assays for enhancer activity (H3K27ac ChIP-seq) and chromatin accessibility (ATAC-seq) on adipose-derived mesenchymal stem cells (AMSCs) from heterozygous individuals across a time course of differentiation (before induction (Day 0), early differentiation (Day 2), intermediate differentiation (Day 6) and terminal differentiation (Day 14)) and compared the numbers of reads from the two haplotypes (FIG. 19b-c). The two haplotypes were associated with a significant difference in H3K27 acetylation, a proxy of enhancer activity, and chromatin accessibility, with the MNOW risk haplotype being enriched by roughly 1.5-fold. The allele specific difference in chromatin accessibility was most pronounced at day 0 of differentiation and declined after induction of differentiation. These results indicate that haplotype 1 is associated with an active enhancer state, while haplotype 2 is associated with a weak enhancer state primarily in adipocyte progenitors.

### Rs6712203 Regulatory Circuitry Affects COBLL1 Gene Expression in Adipocyte Progenitors Conditional on the Transcriptional Regulator POU2F2

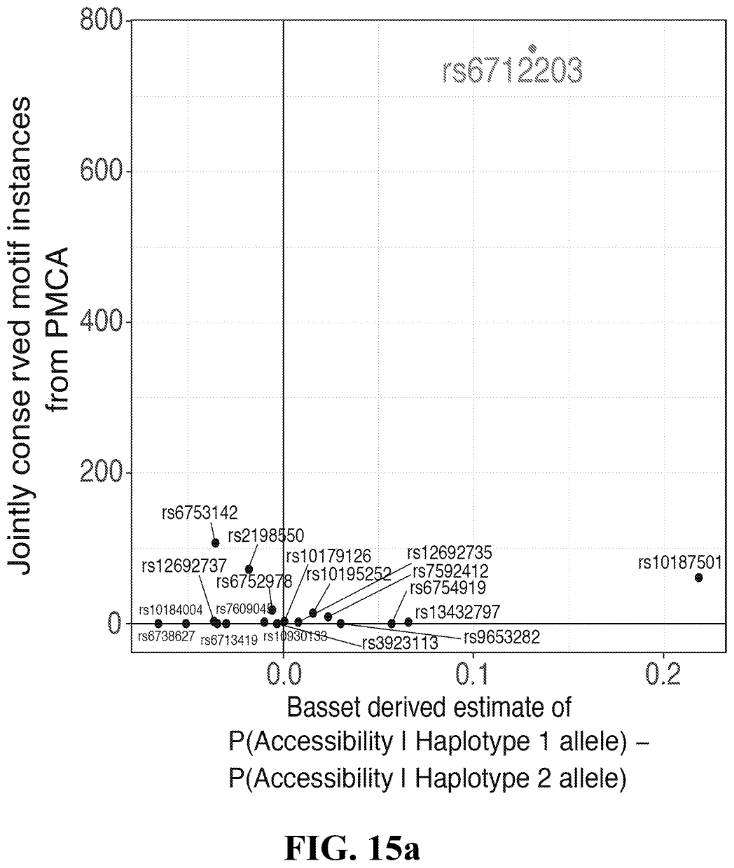

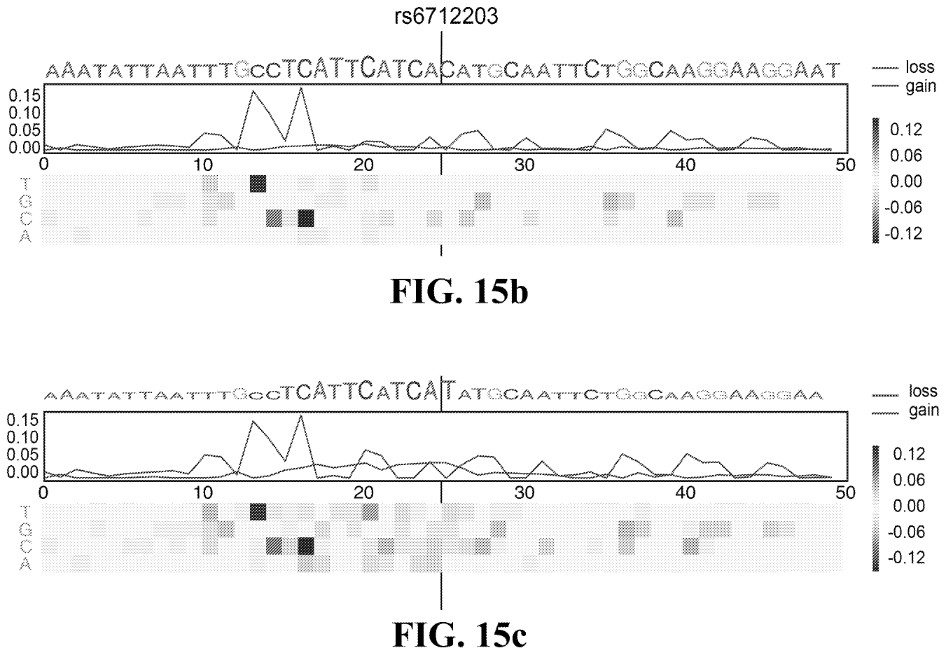

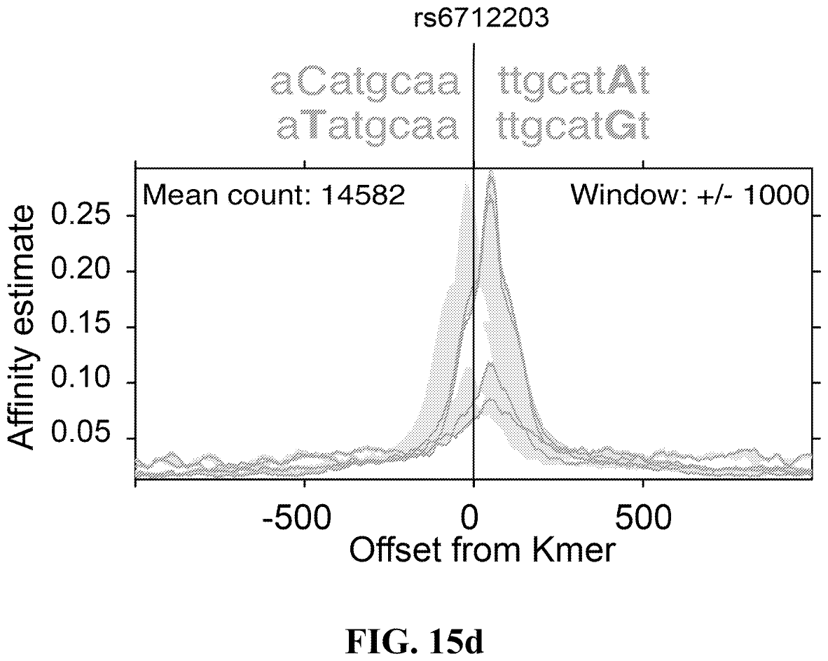

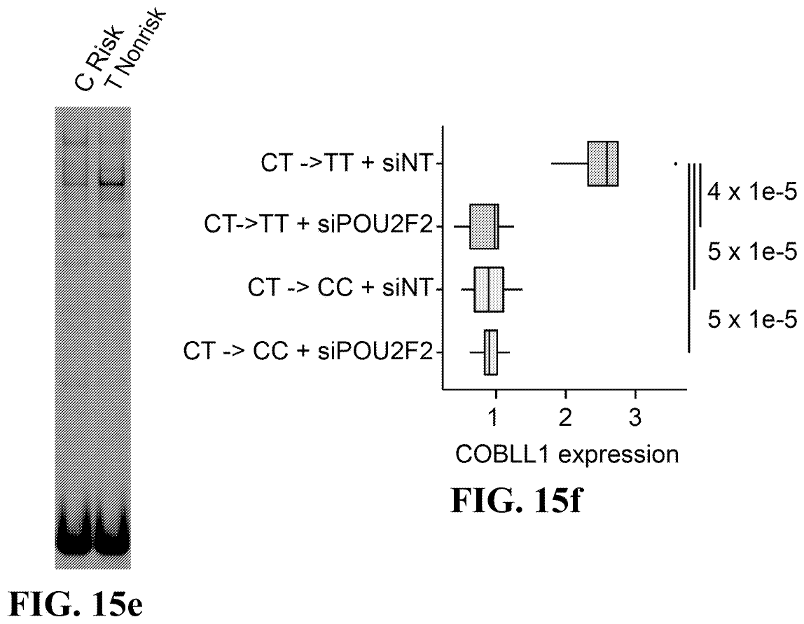

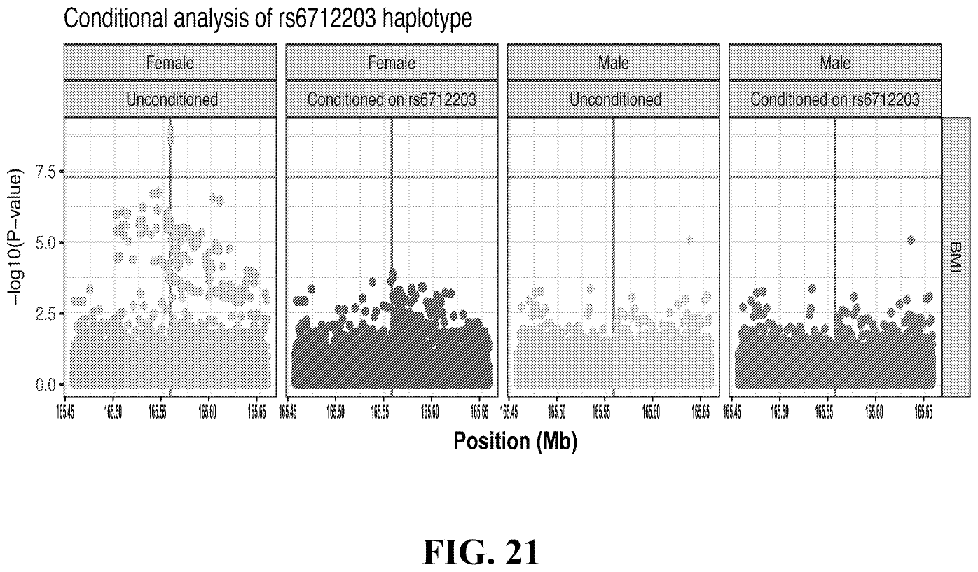

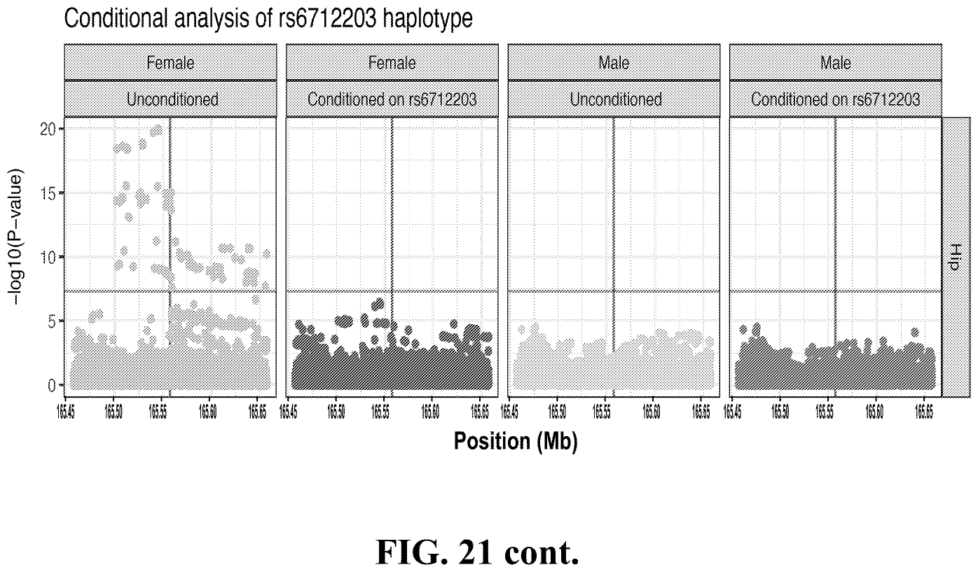

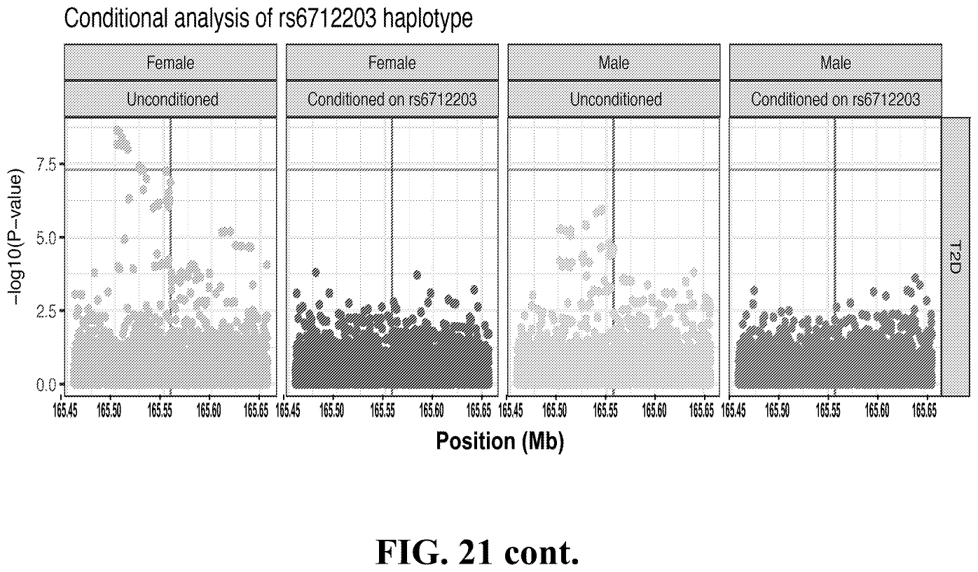

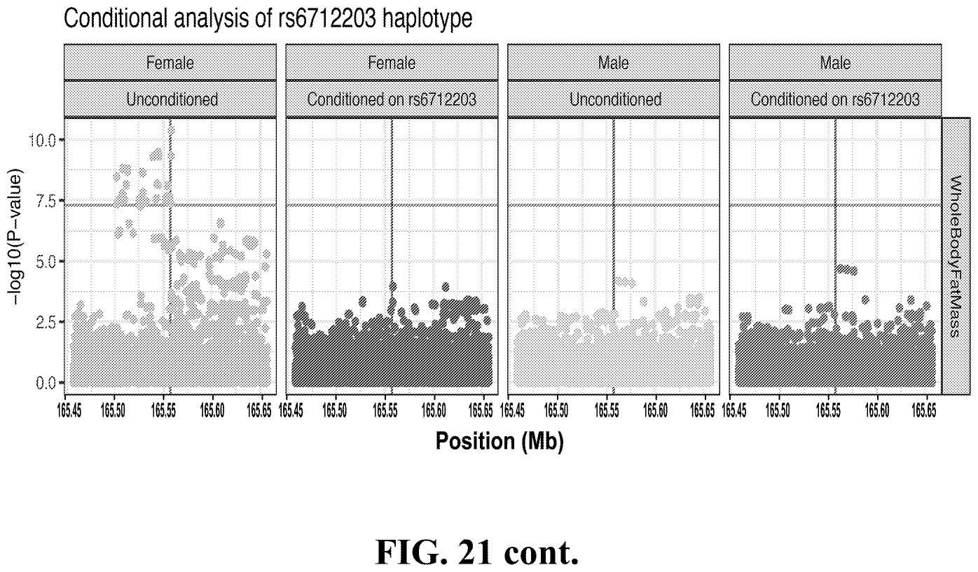

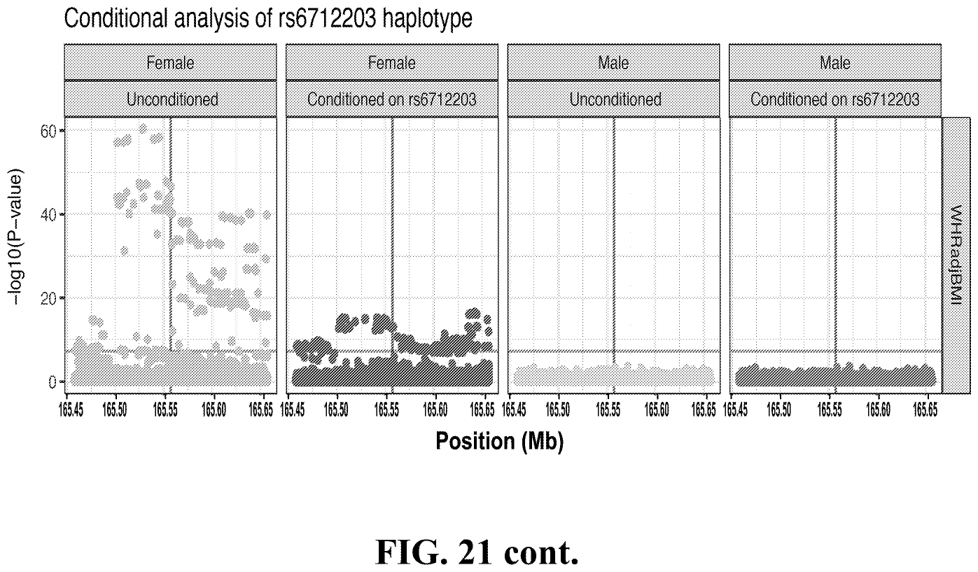

To identify which of the 19 candidate regulatory variants is likely mediating the differential enhancer activity in adipocyte progenitors (FIG. 15a-c), Applicants used two orthogonal computational approaches to prioritize variants, Phylogenetic Module Complexity Analysis (PMCA) (Claussnitzer et al., 2014, 2015; Hindorff et al., 2009) and Bassett (Kelley et al., 2016). PMCA assesses evolutionary conservation of sequence, order and distance (in human and at least one other vertebrate species) of groups of at least three transcription factor binding motifs within a 120 bp-region. Basset (Kelley et al., 2016), uses a sequence-based deep convolutional neural network approach to predict effects of non-coding variants on regulatory activity, by training on the sequence content of a given epigenomic mark in a tissue or cell type of interest. After training on genome-wide chromatin accessibility (ATAC-Seq) data in AMSC progenitors (before induction (DO), one variant, rs6712203, stood out as consistently showing the highest score for PMCA and Bassett (FIG. 15a). Bassett predicted that the T allele on the protective haplotype increases chromatin accessibility relative to the C allele on the risk haplotype in adipocyte progenitors. These sequence-based estimates of rs6712203 C-to-T single nucleotide change importance are consistent with the variant overlapping an active enhancer associated with H3K27 acetylation and H3K4 mono-methylation in adipocyte progenitors. In line with the variant importance at rs6712203, conditional analyses of anthropometric and glycemic traits defining MONW in the UK Biobank confirmed an association consistent with a primary effect driven by rs6712203 C/T in female participants for fat mass and hip circumference and type 2 diabetes in both females and males (FIG. 21). Applicants further observed that the rs6712203 association with T2D was dependent on BMI.

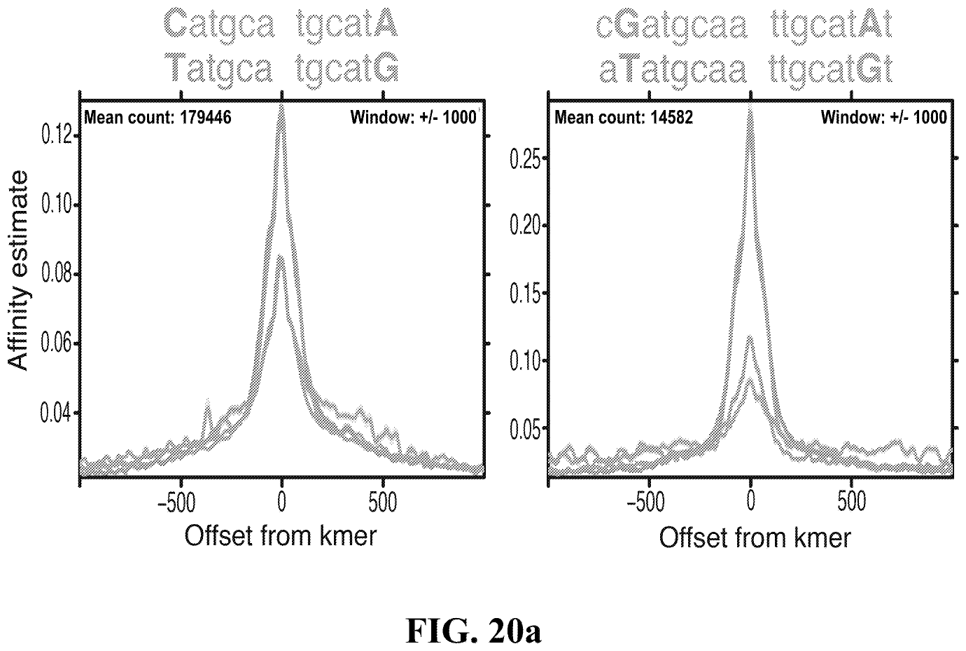

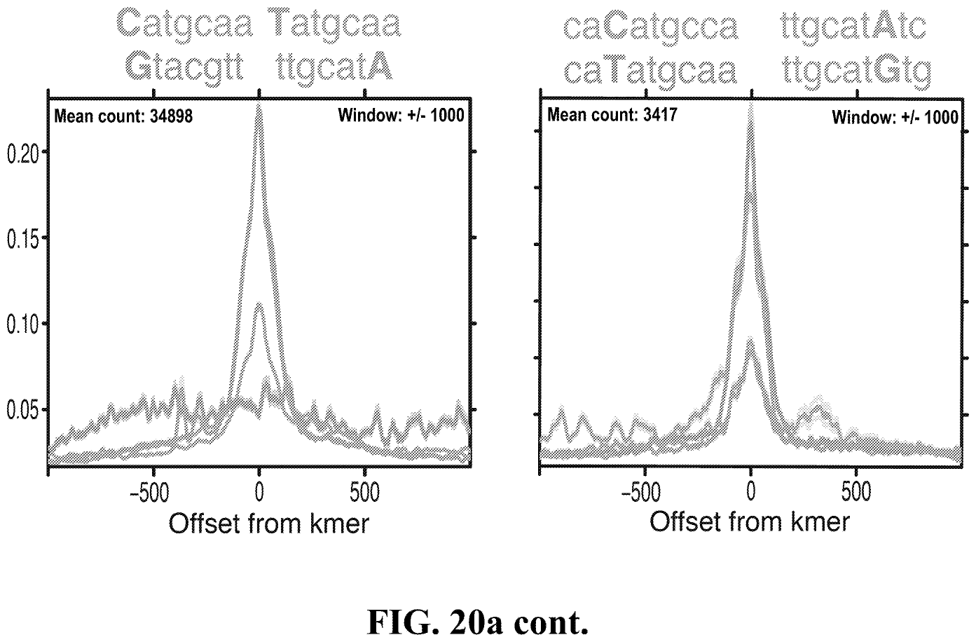

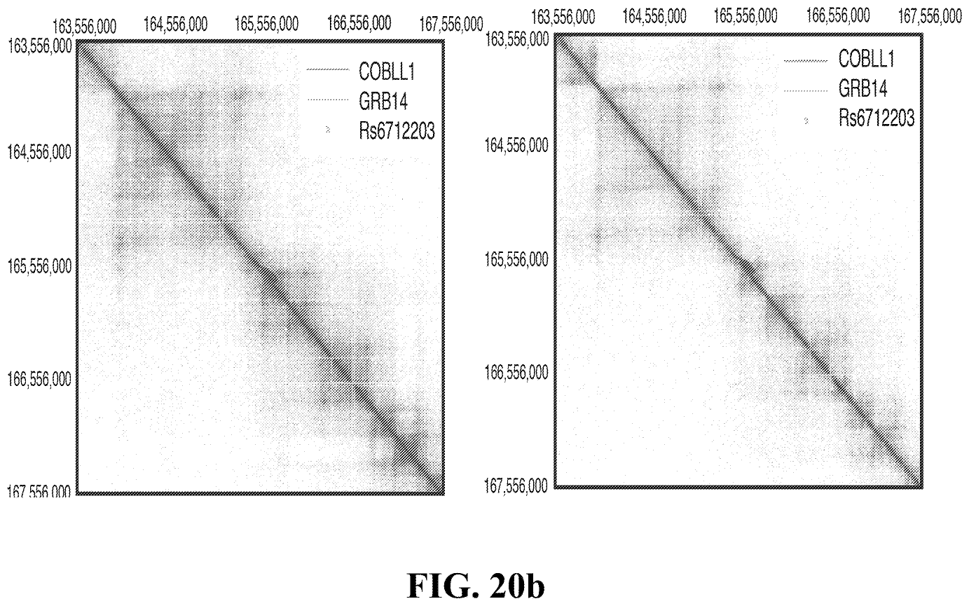

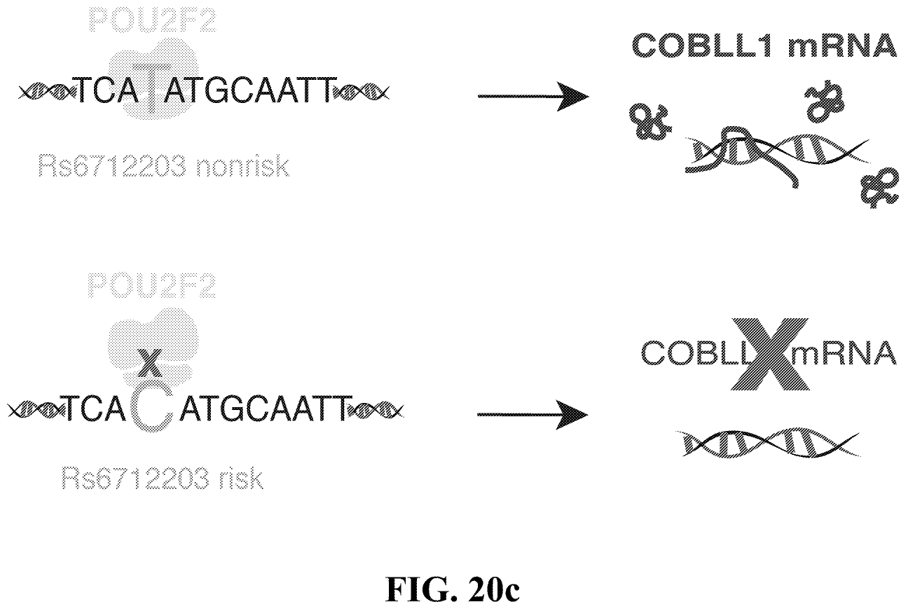

Applicants next performed in silico saturation mutagenesis to evaluate the predicted change in chromatin accessibility from mutation at every position to each alternative nucleotide within a 20 bp region surrounding rs6712203 using ATAC-Seq data during AMSC differentiation. Applicants found that the rs6712203 T allele is critical for a POU2F2 motif (FIG. 15b-d). The C allele of this SNP converts the chromatin in this site into less accessible, supporting a model in which a transcription factor, possibly POU2F2, differentially binds to these allelic variants of rs6712203. To estimate preferential binding affinity of POU2F2 to the C risk compared to the T non-risk allele, Applicants used the intragenomic replicate (IGR) method (Cowper-Sal-lari et al., 2012) on publicly available POU2F2 ChIP-seq data from the ENCODE project. By comparing the frequency of k-mers matching the rs6712203 T allele versus the C allele, Applicants confirm that POU2F2 preferentially binds to the T allele (9-mer change in affinity-0.38, two-tailed permutation p<0.0034) (FIG. 15d, FIG. 20a). Applicants further confirmed that the rs6712203 T-to-C nucleotide change alters transcription factor binding by performing allele-specific electrophoretic mobility shift assays (EMSA) using nuclear lysates from adipocyte progenitors (FIG. 15e). These data indicate an increased POU2F2 binding to the rs6712203 T non-risk allele and suggest POU2F2 as the upstream regulator of variant action at this locus.

Applicants next sought to establish rs6712203 causality by directly confirming that the haplotype-specific effects on enhancer activity and POU2F2 binding are mediated by rs6712203 using CRISPR-based genome editing at this SNP. Applicants edited SGBS preadipocytes (n=5) that are heterozygous at rs6712203 to create isogenic lines for the TT (non-risk genotype) and CC (risk genotype) alleles. Applicants observed that cells harboring the CC homozygous risk showed 2.4-fold lower COBLL1 expression levels compared to the TT non-risk genotype (FIG. 15f), pointing towards COBLL1 as a target gene of the rs6712203 regulatory circuitry. To test a cis-/trans-conditional effect of the variant rs6712203 and the upstream regulator POU2F2 on target gene expression Applicants performed targeted regulator knockdown by siRNA mediated ablation of POU2F2 in AMSCs and found that silencing of POU2F2 in TT allele carriers reduces COBLL1 gene expression to the level of CC allele carriers in preadipocytes (FIG. 15f), confirming POU2F2 as a crucial regulator at this locus.

Applicants used three-dimensional genome conformation data from Hi-C assays in embryonic stem cell-derived MSCs (Dixon et al. 2015) to define the physical boundaries of potential proximal and long-distant target genes and found that the locus lies in a well-defined contact domain containing only two genes, COBLL1 and GRB14 (FIG. 15b), without any evidence for long-range chromatin interactions. Together Applicants found that rs6712203 T-to-C editing in AMSCs reduces COBLL1 gene expression in a POU2F2-dependent manner (model schematic in FIG. 20c).

### COBLL1 Affects Actin Remodeling in Subcutaneous Adipocytes

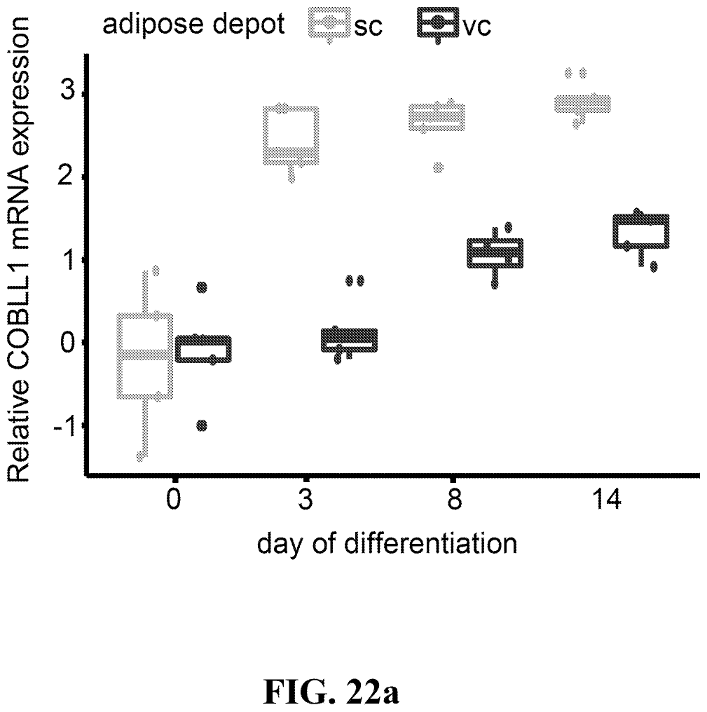

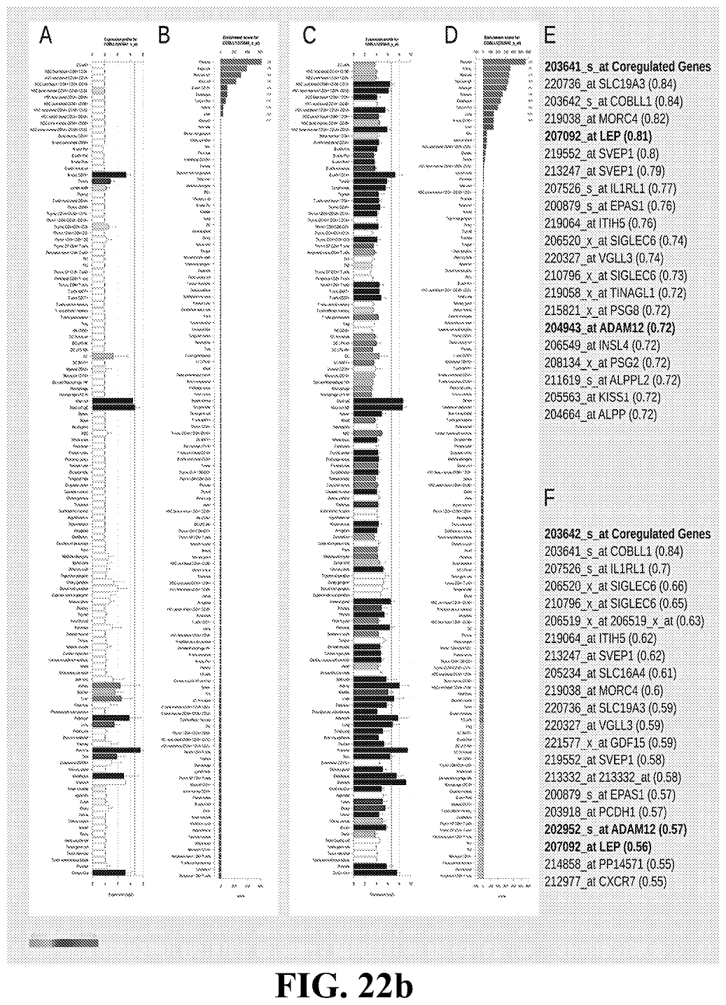

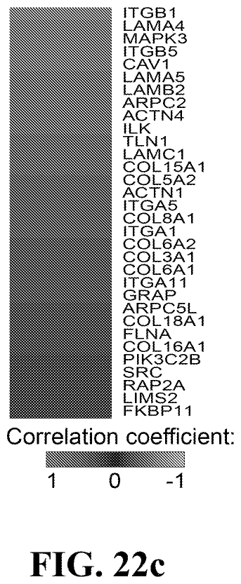

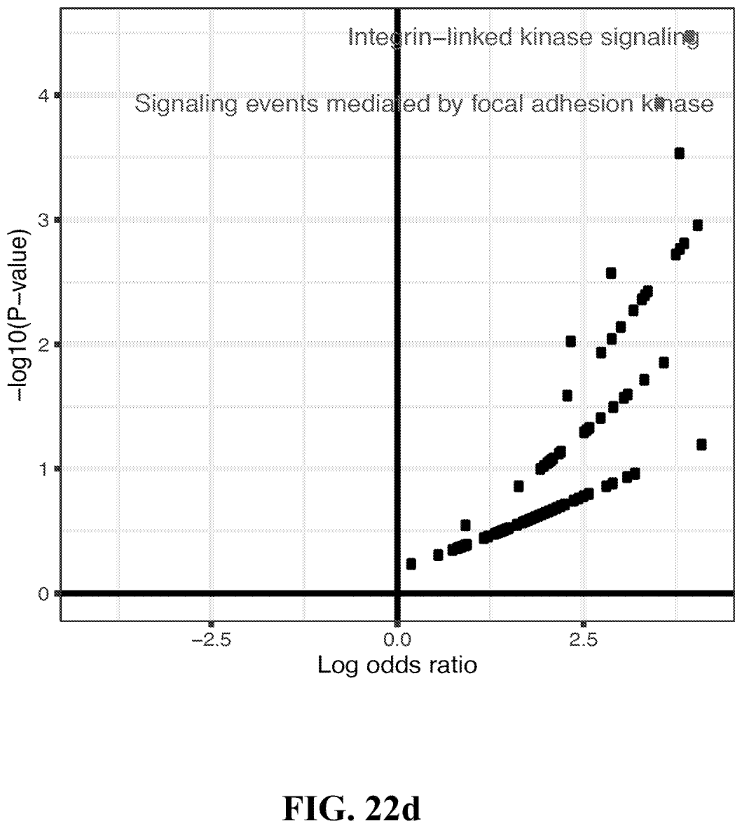

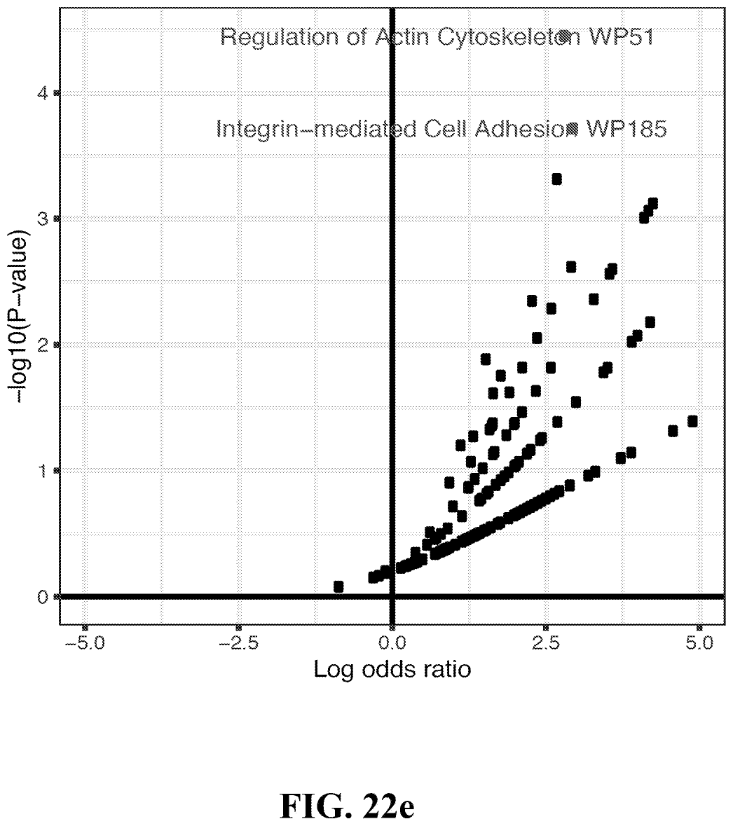

To understand the role of COBLL1 in adipocyte cellular programs, Applicants first examined the gene expression and cellular localization of COBLL1 in differentiating adipocytes and observed that COBLL1 is expressed at any given stage of adipocyte differentiation with an increase in mRNA and protein levels over the course of differentiation (FIG. 22a). Applicants observed consistently higher COBLL1 mRNA levels in subcutaneous compared to visceral adipocytes across the adipocyte differentiation process (FIG. 22a). Overall, Applicants found an enrichment of COBLL1 gene expression in adipose compared to 146 other tissues and cell types (Benita et al. 2010) (FIG. 22b).

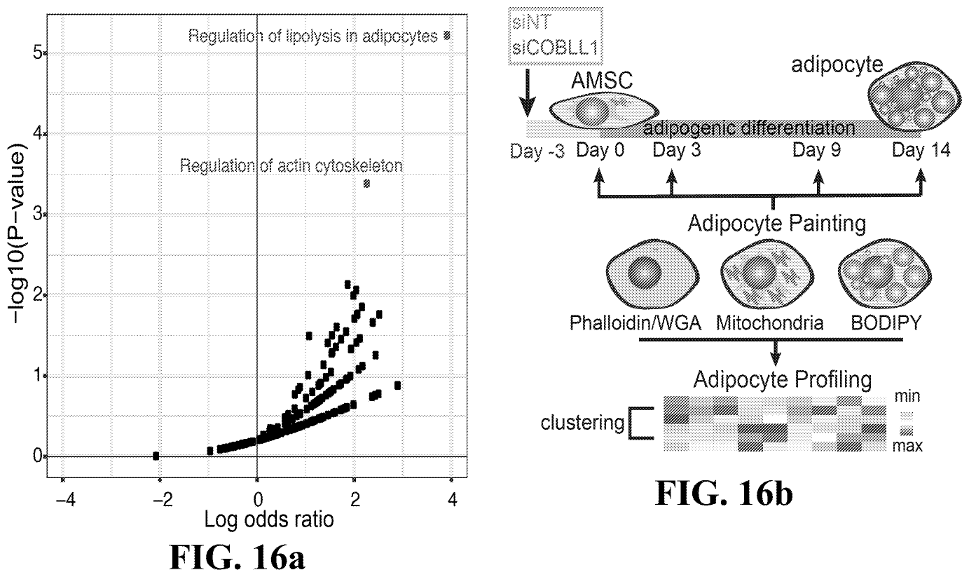

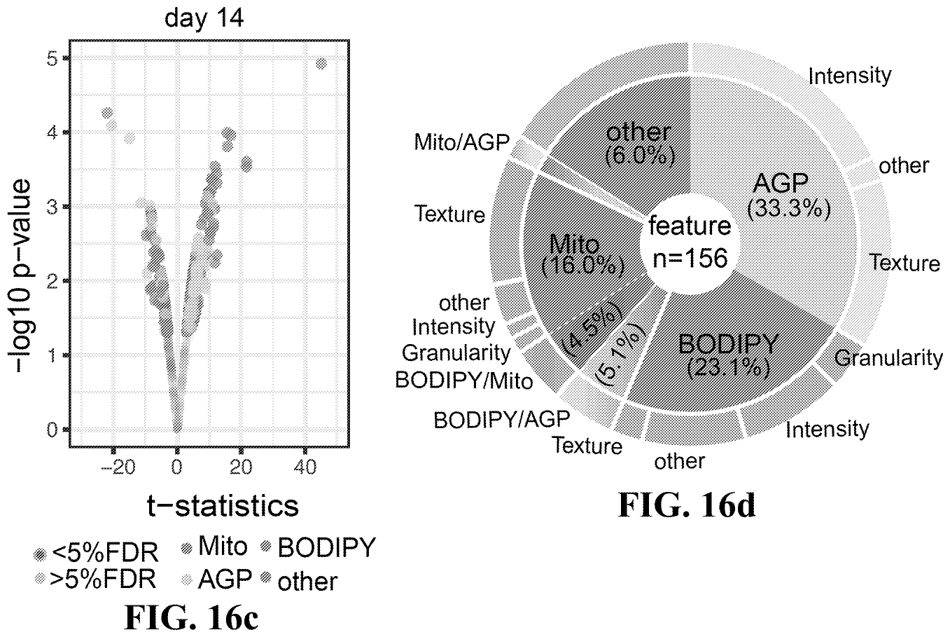

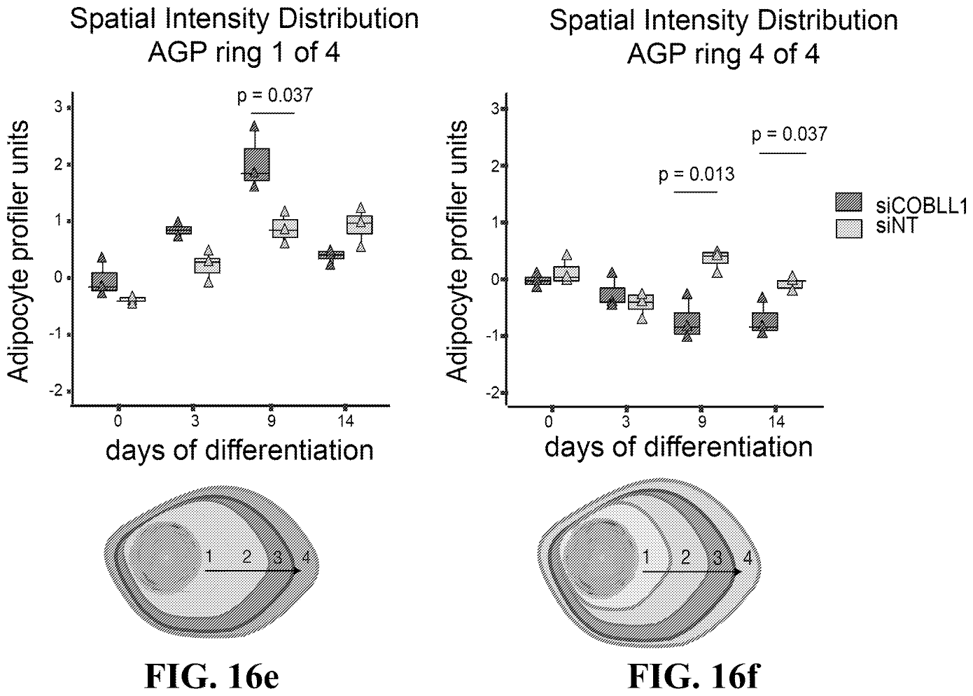

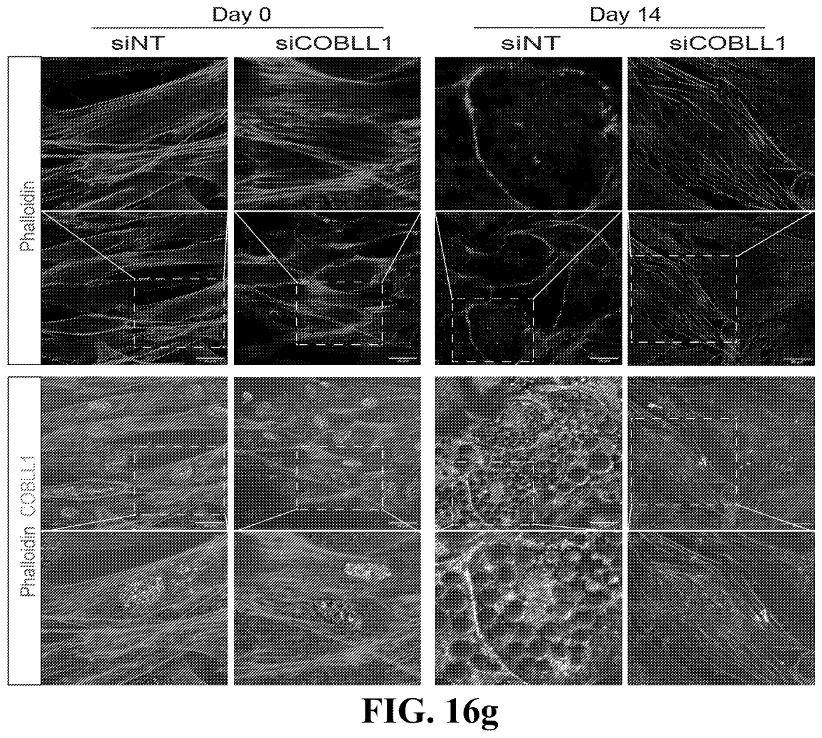

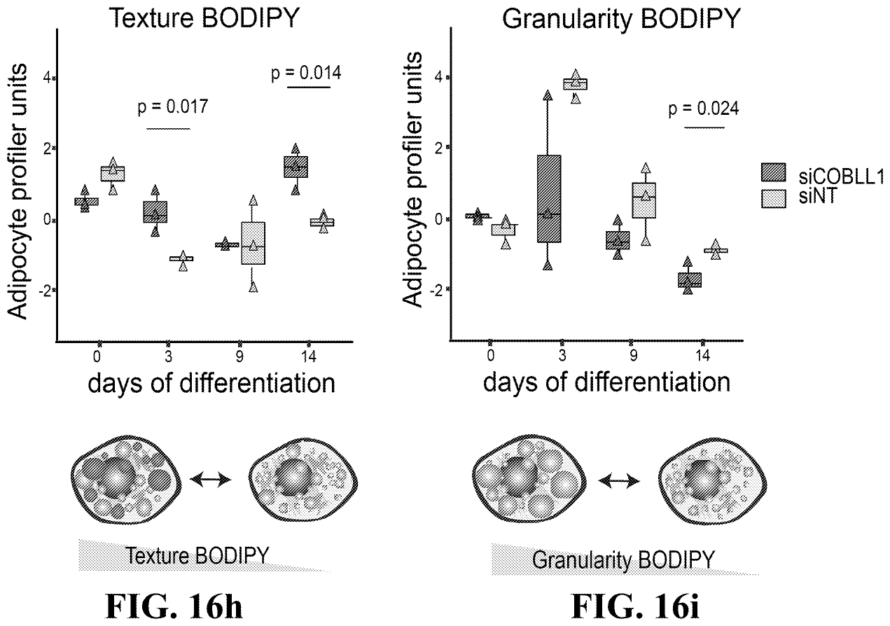

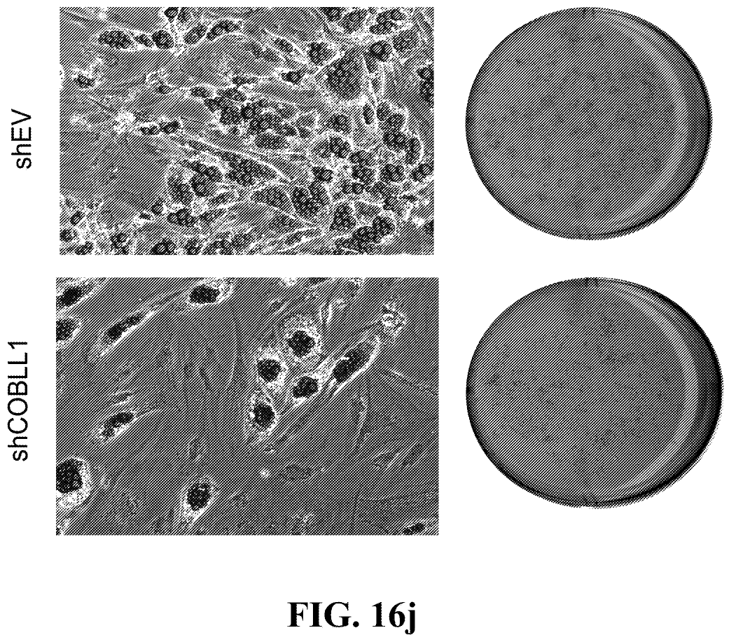

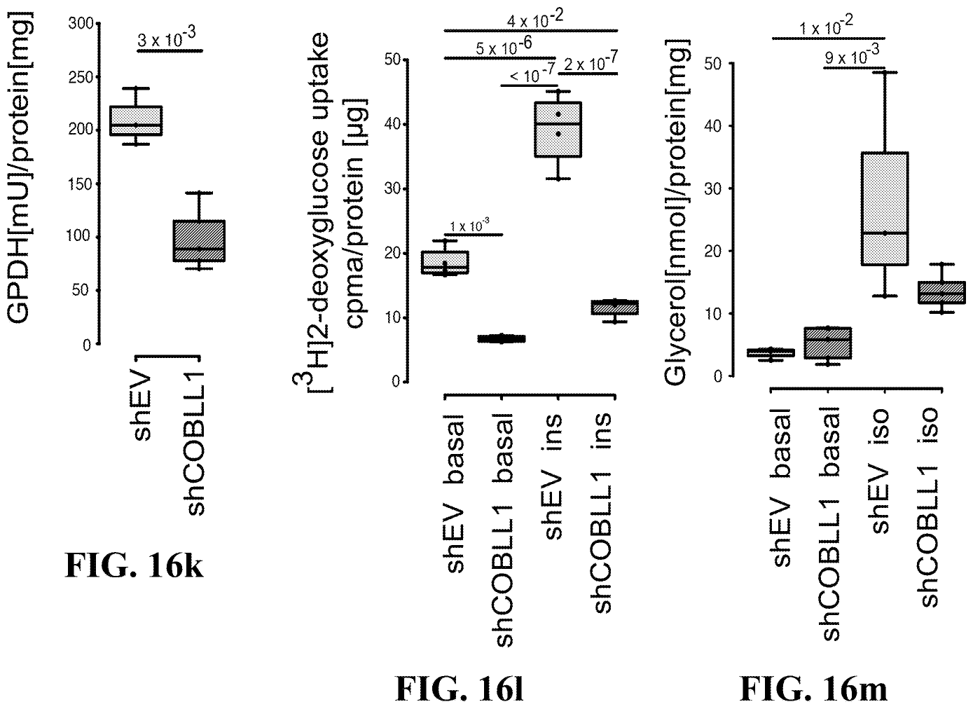

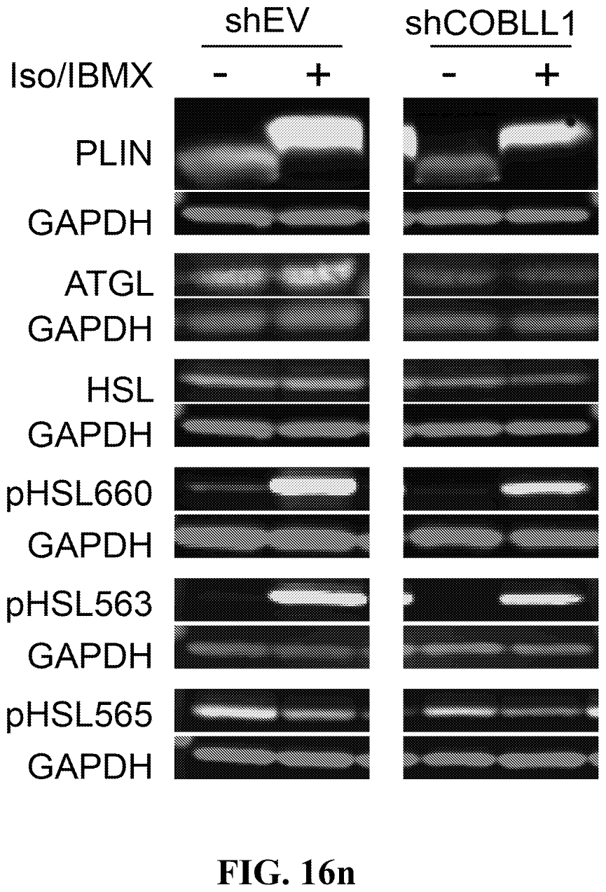

To connect the 2q24.3 locus to cellular functions in adipose Applicants used genome-wide co-expression matrices in adipocytes matched with a series of cellular assays. Applicants identified COBLL1 co-regulated genes in genome-wide expression data from primary human AMSCs in a cohort of 12 healthy, non-obese individuals. COBLL1 co-expressed genes were highly enriched in biological processes related to ‘Regulation of actin cytoskeleton’ and ‘Regulation of lipolysis in adipocytes’, including ITGAM (Integrin Subunit Alpha M), PIK3CA (Phosphatidylinositol-4,5-Bisphosphate 3-Kinase Catalytic Subunit Alpha), ROCK2 (Rho-associated protein kinase 2), ITGA1 (Integrin alpha-1), ARHGEF7 (Rho Guanine Nucleotide Exchange Factor 7), CRK, FGFR2 (Fibroblast Growth Factor Receptor 2), ARHGEF6 (Rho Guanine Nucleotide Exchange Factor 6) (FIG. 16a, FIG. 22c-e, Tables 10-13), which are implicated in actin remodeling processes and insulin responsiveness (Kawaguchi et al. 2003; Qian et al. 2004); Morandi et al. 2016; Trucbestein et al. 2015). This is consistent with recent studies showing that COBLL1 possesses a single WH2 (Wiskott-Aldrich syndrome protein homology 2) actin monomer-binding domain, and promotes F-actin formation in Cos-7 and neuronal cells and prostate cancer cells (Izadi et al. 2018; Takayama et al. 2018).

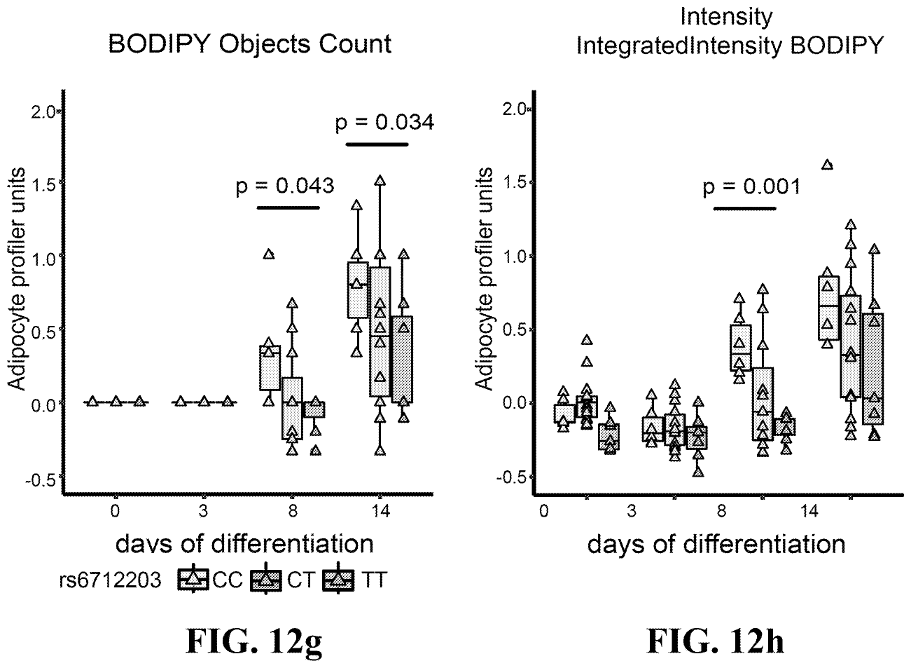

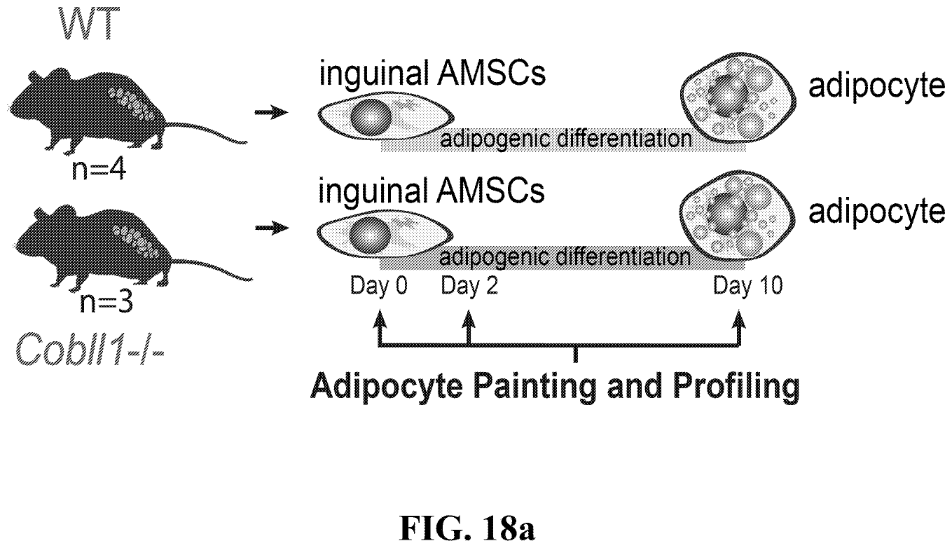

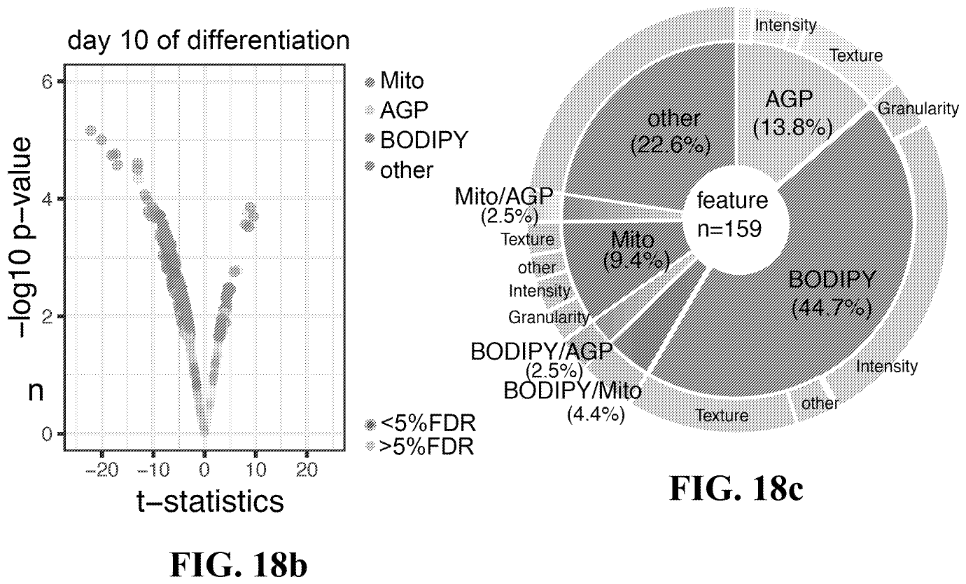

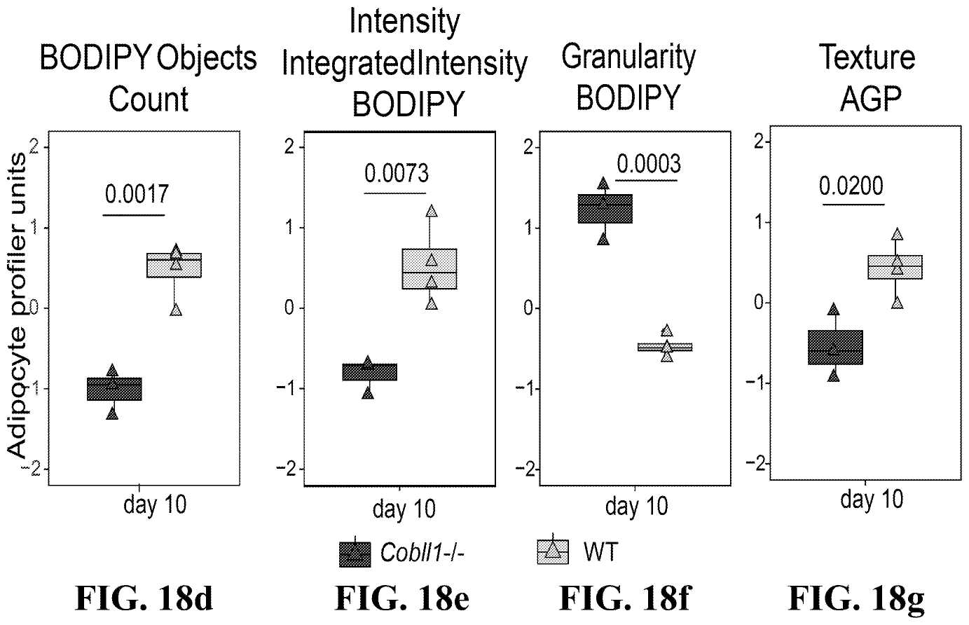

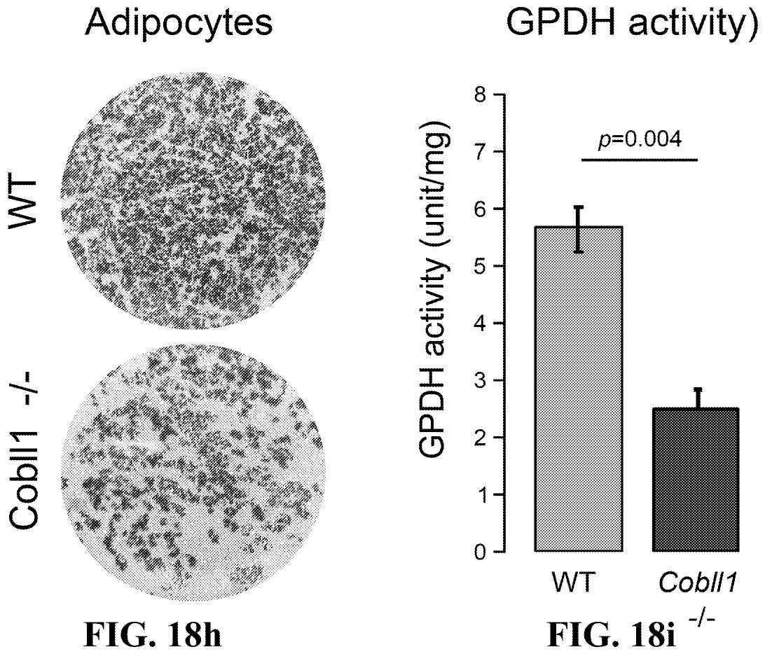

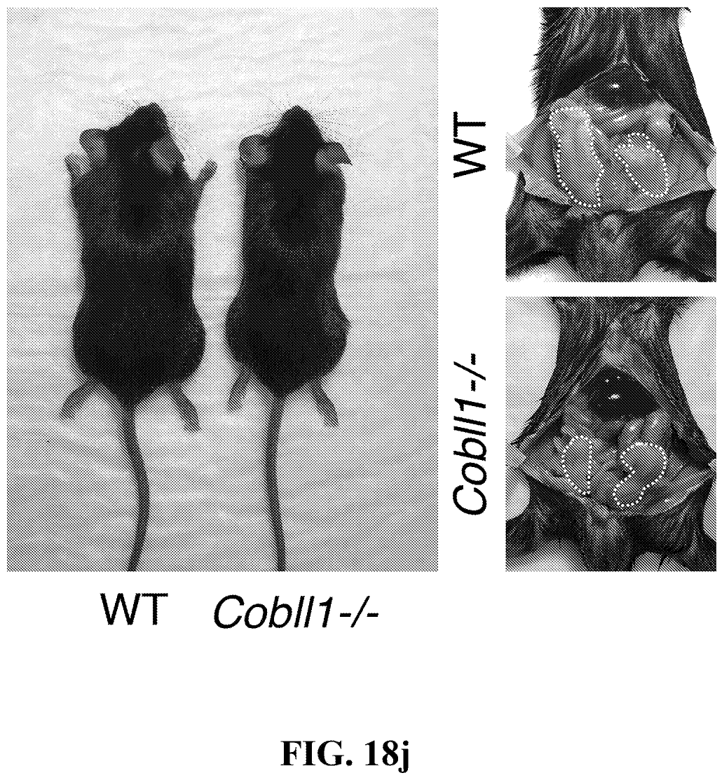

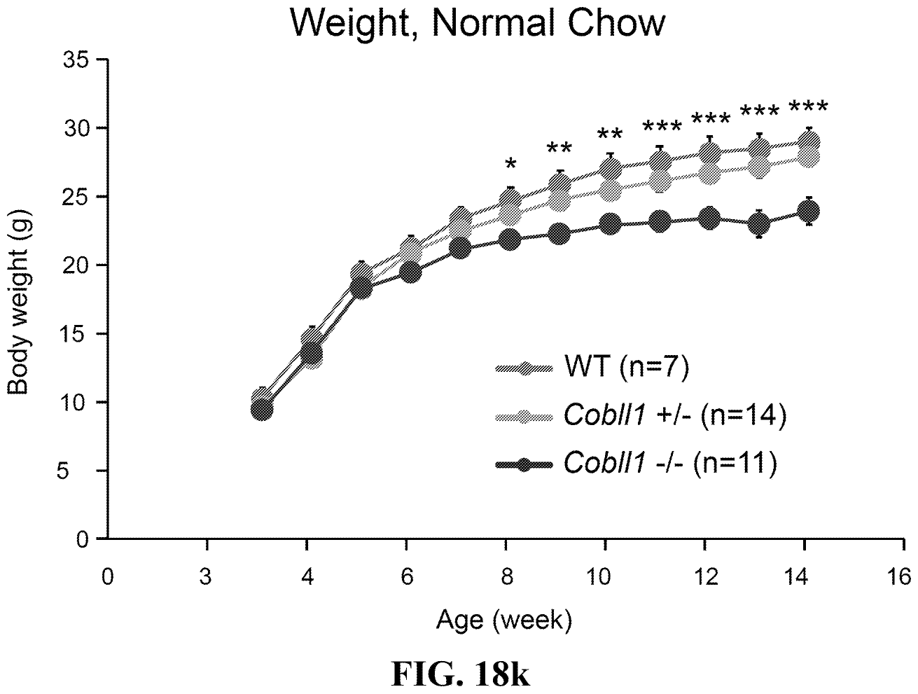

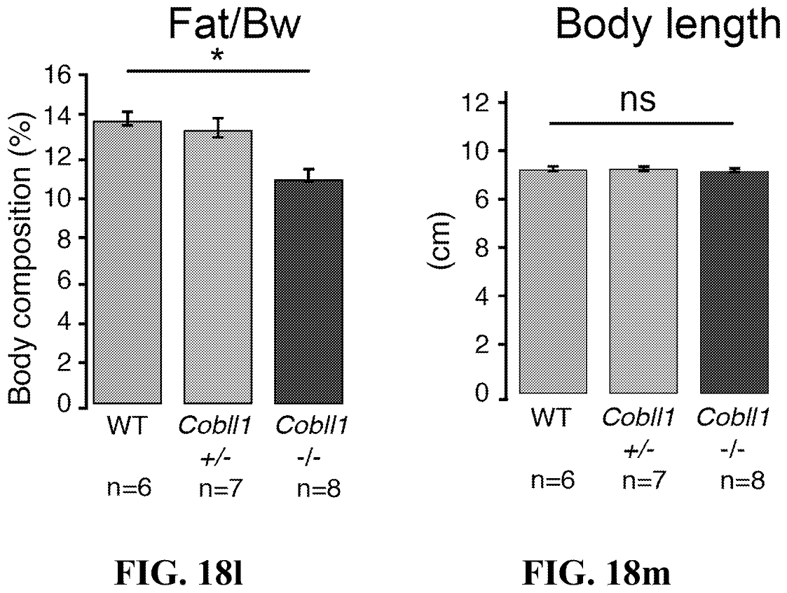

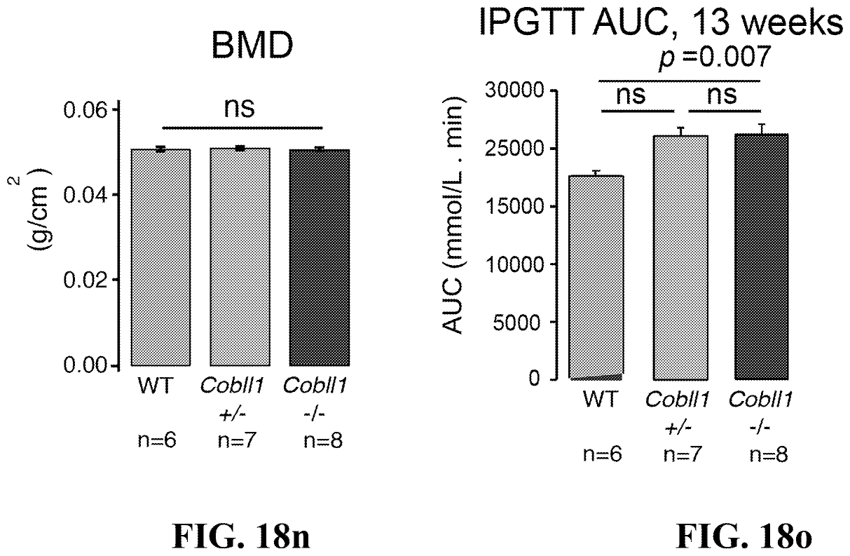

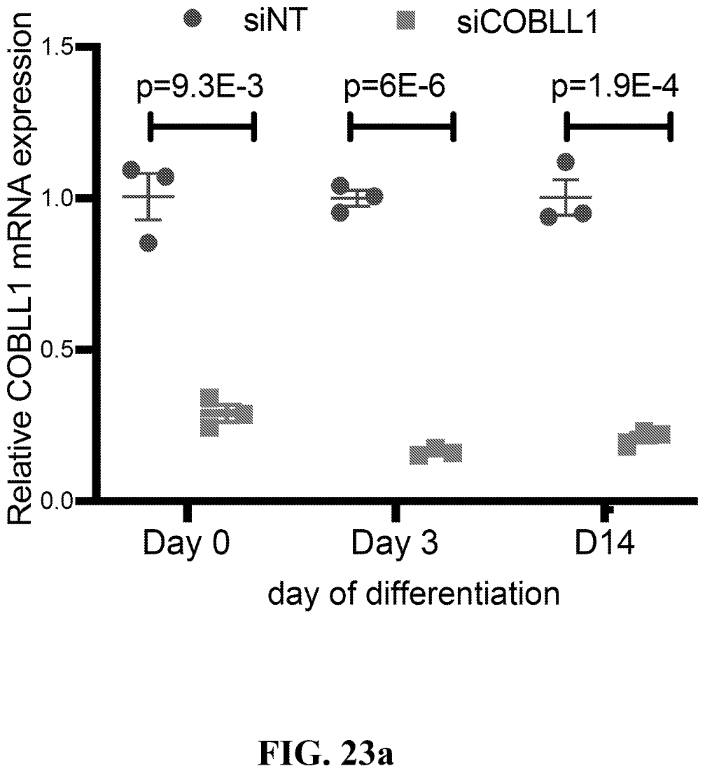

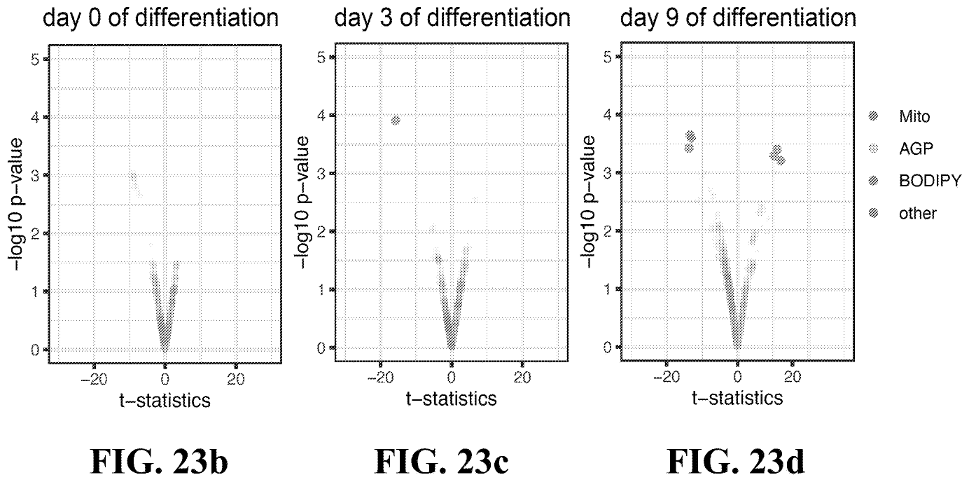

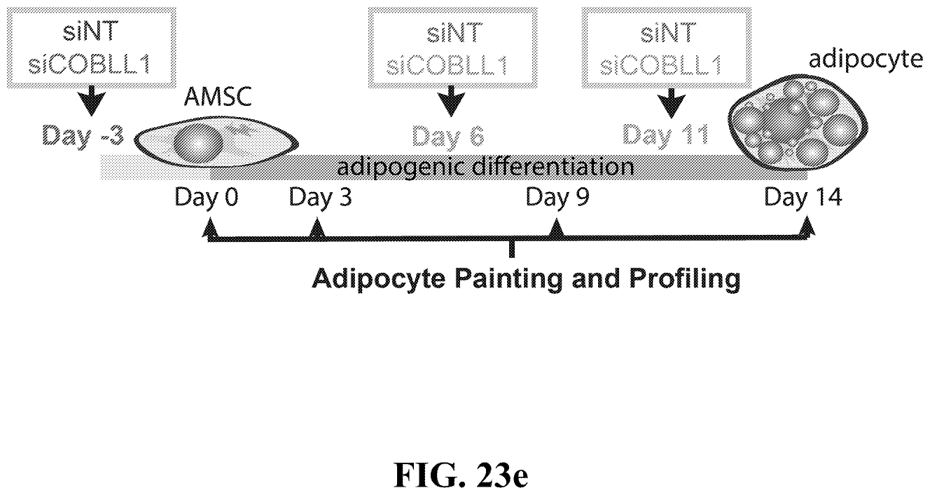

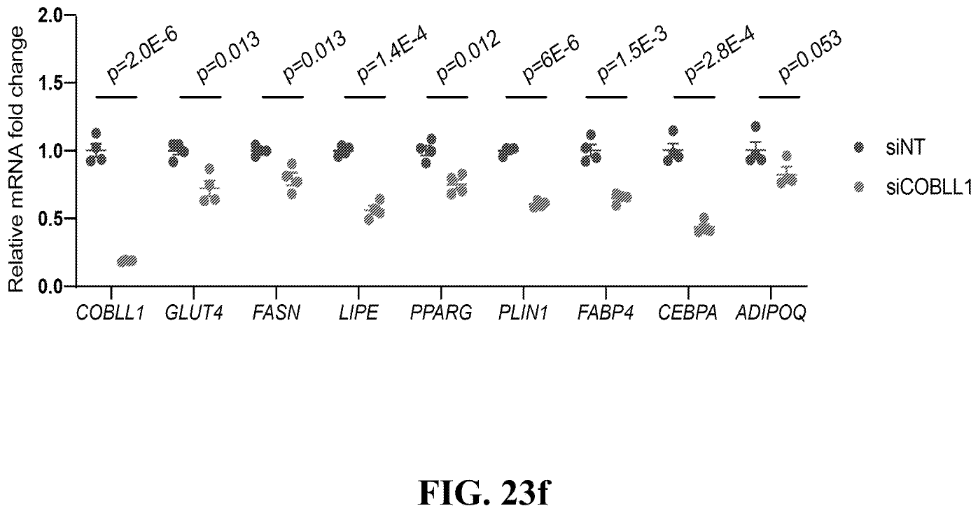

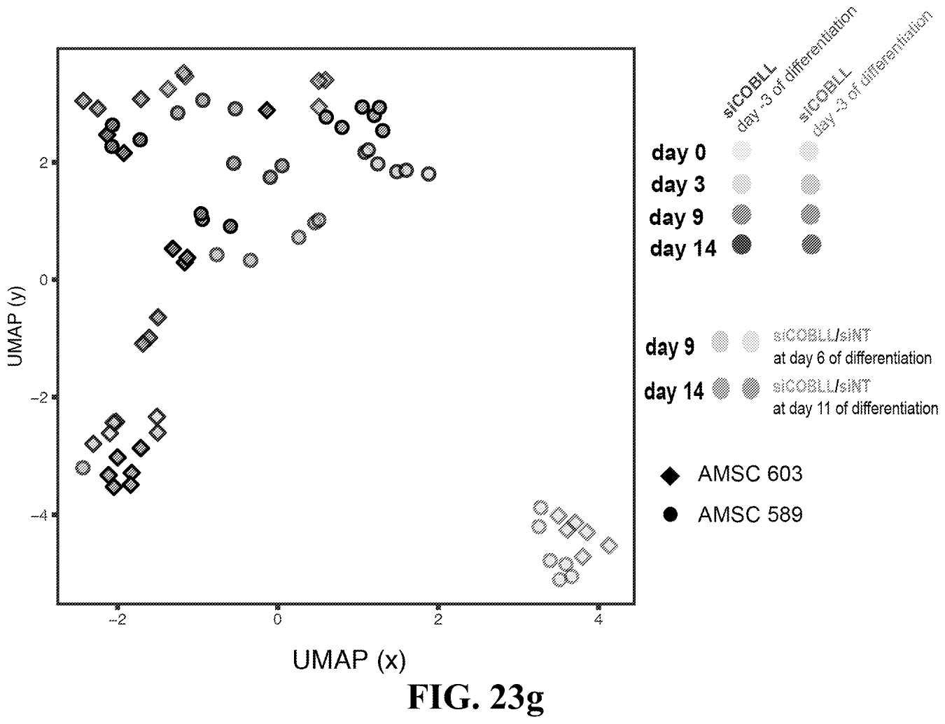

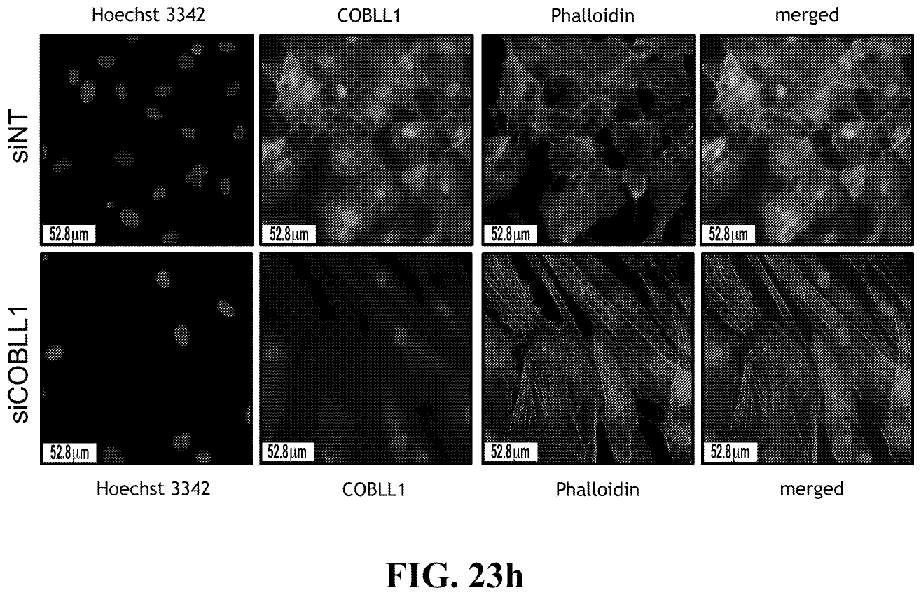

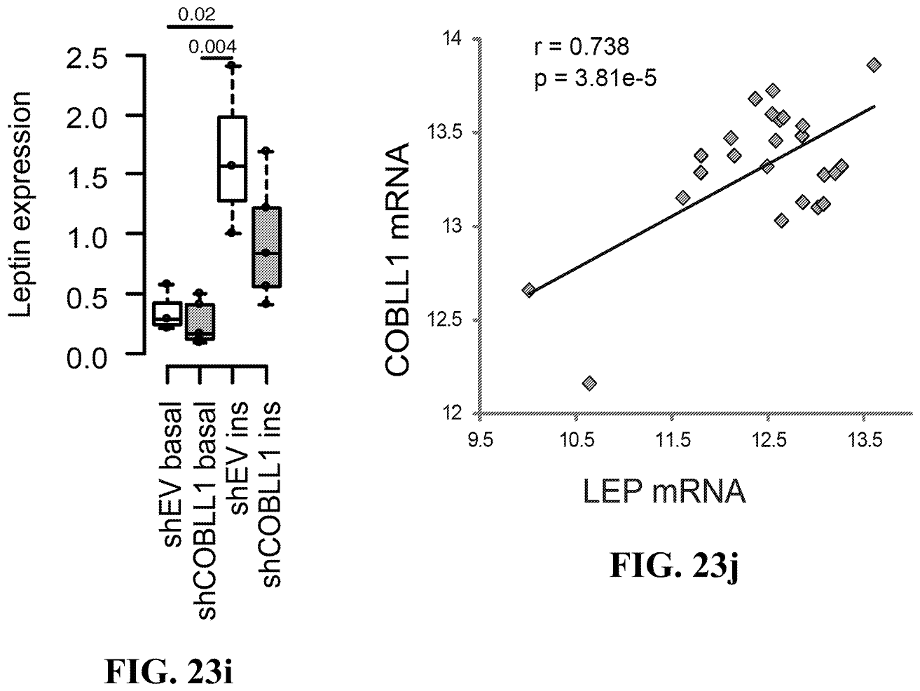

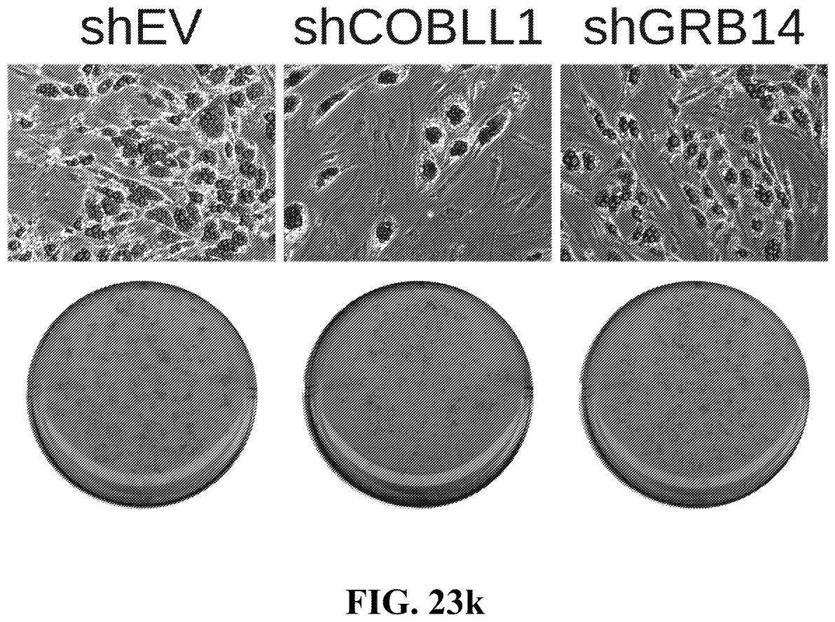

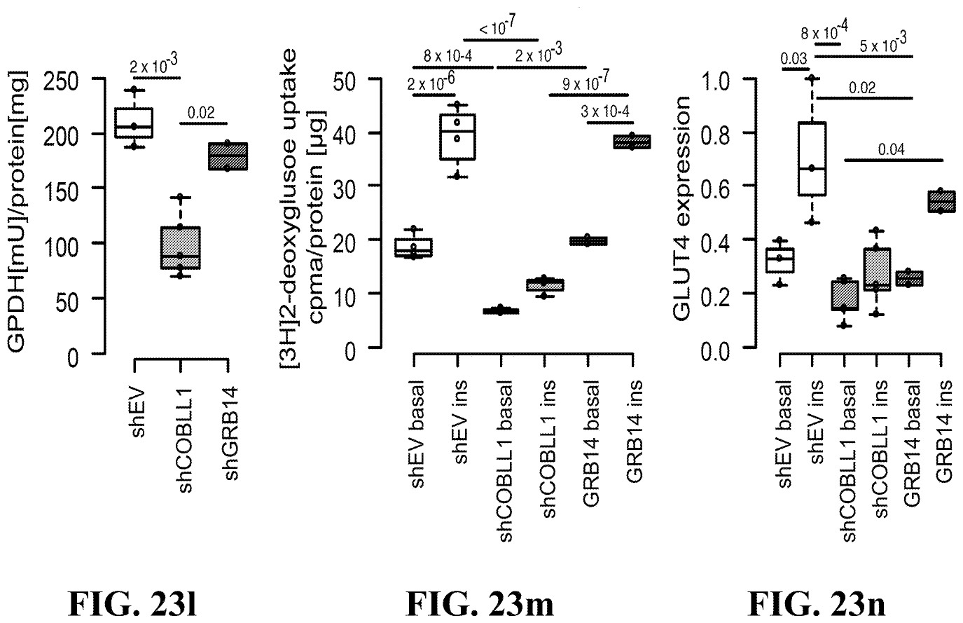

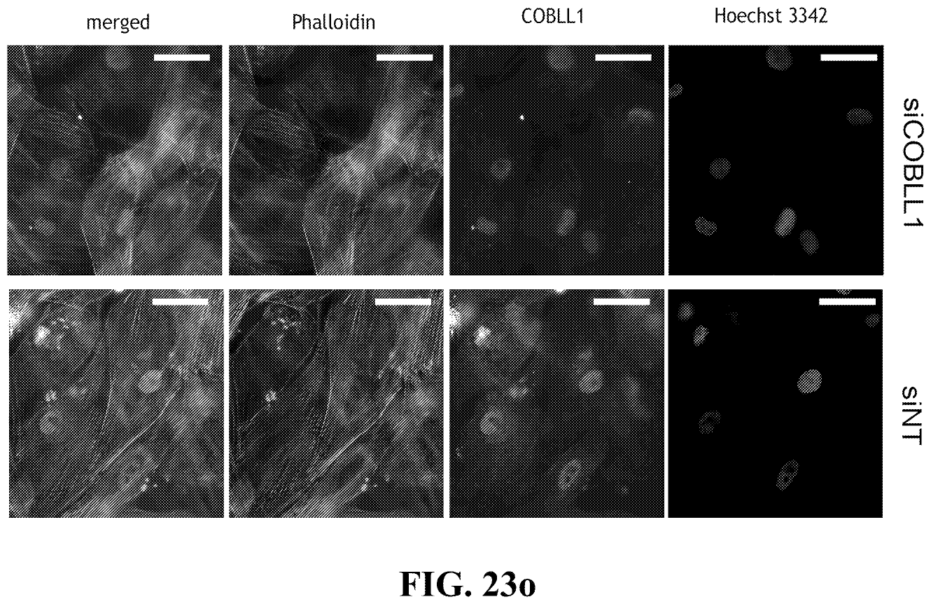

To identify morphological and cellular traits associated with altered COBLL1 expression, Applicants used siRNA-mediated knockdown of COBLL1 in AMSCs coupled with a high-content imaging read-out that Applicants recently developed, Adipocyte Profiler (Sec Example BioRXiv). Adipocyte Profiler allows to examine generic as well as adipocyte-specific cellular traits at four time-points of adipocyte differentiation (before differentiation (day 0), three days (day 3), nine days (day 9) and 14 days (day 14) after adipogenic induction) (FIG. 16b). Applicants examined 1175 quantitative features, spread across two cellular compartments (cell and cytoplasm) and five dyes informative for morphological and adipocyte cellular traits (BODIPY, Phalloidin, WGA, SYTO14, MitoTracker, see Methods) imaged in four fluorescence channels (FIG. 16b). Applicants observed that COBLL1 knockdown in proliferating pre-adipocytes (three days before induction of adipogenesis) with 80% knockdown efficiency (FIG. 23a) results in changes of diverse morphological and cellular features across adipocyte differentiation with a peak at later stages of differentiation (FIG. 16c, FIG. 18b-d). On day 14 of differentiation 156 features differed significantly (FDR<5%) between COBLL1 knockdown and non-targeting control, spread across BODIPY (23.1%), actin-related (AGP) (33.3%) and mitochondrial (16%) channels (FIG. 16d, Table 14). For actin related cellular processes, Applicants observed that COBLL1 knockdown results in differences of spatial intensity distribution of AGP across the cytoplasm. Following COBLL1 silencing Applicants observed increased actin-associated intensity in the center of the cell (day 9p=0.037, FIG. 16e) and decreased actin-associated intensity at the cell cortex (day 9p=0.013, day 14 p=0.037, FIG. 16f) in terminally differentiated subcutaneous adipocytes. This indicates that COBLL1 plays a role in the remodeling of the actin cytoskeleton, as reduced levels of COBLL1 disturb the disassembling of filamentous actin (F-actin) stress fibers across the cytoplasm and the reassembling to cortical F-actin (F-actin juxtaposed to the plasma membrane) during adipocyte maturation, which was accompanied by a reduction in differentiation capacity as shown with decreased amount of lipid droplet formation. More specifically, Applicants confirmed that the COBLL1 knockdown was associated with a decreased disruption of stiff F-actin stress fibers reaching in the middle of the cell body at the expense of F-actin structure assembly at the cell cortex in differentiated cells (FIG. 16g, FIG. 23h). Consistent with the notion that remodeling of F-actin stress fibers to cortical actin is linked to adipocyte differentiation Applicants observed that COBLL1 ablated adipocytes have both a smoother texture of BODIPY-related pixels (pixel intensities are more similar, day 3 p=0.017 and day 14 p=0.014) and a lower BODIPY-related granularity (smaller size spectra of BODIPY objects, day 14 p=0.024) within the cell compared to adipocytes expressing COBLL1, which is indicative of disturbed lipid droplet formation and adipogenic differentiation in COBLL1 ablated cells (FIG. 16h-i).

To investigate if the COBLL1 effect on actin remodeling in adipocytes impacts adipocyte cellular programs related to metabolic disease, Applicants performed stable ablation of COBLL1 using lentivirus (shCOBLL1) in differentiating adipocytes. Applicants observed that ablation of COBLL1 resulted in decreased capacity to differentiate into metabolically active round-shaped lipid filled mature adipocytes, as shown by decreased Oil-Red-O staining of accumulated triglycerides (FIG. 16j), adipocyte differentiation marker gene expression (FIG. 23F) and glycerol-3-phosphate dehydrogenase (GPDH) activity measurements (2.2-fold, p=0.04, FIG. 3k). Applicants further found a correlation between the mRNA levels of COBLL1 and leptin, an adipokine produced in proportion to the size of fat depots (Harris 2014) in primary isolated subcutaneous floating adipocytes (r=0.74, p-value=5×10⁵) (FIG. 23i-j). This effect on leptin is consistent with genome-wide association studies of serum leptin levels (rs6712203 C allele beta-0.0308, p=9×10⁻⁶ and beta-0.0236, p=1×10⁻⁵ [BMI-adjusted] in Kilpeläinen et al 2016 (Kilpeläinen et al. 2016); and beta 0.0285, p 0.005889 in Folkersen et al 2020 (Folkersen et al. 2020)). Applicants further found a 2.1-fold (p-value 2×10⁻⁵) decrease of insulin-responsive glucose uptake in shCOBLL1 adipocytes compared to non-targeting control, as measured by radiolabeled 2-deoxyglucose uptake assays (FIG. 16i). In fact, the data revealed a lack of shCOBLL1 adipocytes to respond to insulin presumably mediated likely as a result of both a decreased differentiation efficiency as well as a failure of the cortical actin remodeling mediated GLUT4 vesicle trafficking. Finally, Applicants observed a failure of shCOBLL1 adipocytes to break down triglycerides to free fatty acids and glycerol through lipolysis following β-adrenergic stimulation using isoproterenol and phosphodiesterase inhibitor IBMX compared to their control cells (FIG. 16m). This was accompanied by decreased protein levels of the lipolytic enzymes adipocyte triglyceride lipase (ATGL), hormone sensitive lipase (HSL), PKA Serine phosphorylated HSL (pHSL660, pHSL563) and on the lipid droplet-associated protein perilipin (PLIN) (FIG. 16n). Notably, Applicants did not observe an effect on cellular and morphological features when COBLL1 was silenced after induction of differentiation (FIG. 23e,g), suggesting that COBLL1 acts early in differentiation with phenotypic effects primarily manifesting in mature adipocytes. Applicants also did not observe an effect when COBLL1 was ablated in visceral AMSCs (FIG. 23n), indicating that COBLL1 is critically involved in actin remodeling processes in subcutaneous adipocytes.

Applicants further examined the effect of GRB14 stable knockdown in AMSCs and observed that GRB14 ablation did not significantly decrease adipocyte differentiation capacity as measured by Oil-Red-O staining, GPDH activity (FIG. 23k-l), and insulin-responsive glucose uptake (FIG. 23n), supporting COBLL1 as the effector gene at this locus.

Together, Applicants connect COBLL1, an 2q24 effector gene, to actin cytoskeleton remodeling processes in differentiating subcutaneous adipocytes, accompanied by a failure in adipocyte differentiation and function, including increased glucose uptake in response to insulin, and lipid break-down to free fatty acids.

### The Rs6712203 MONW Risk Haplotype Affects Actin Cytoskeleton Remodeling and Adipocyte Function

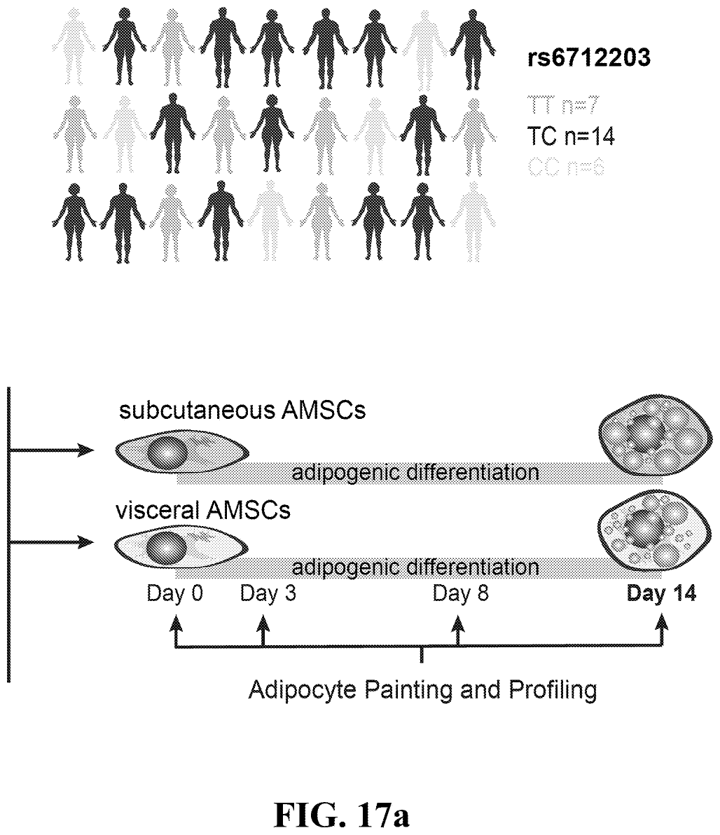

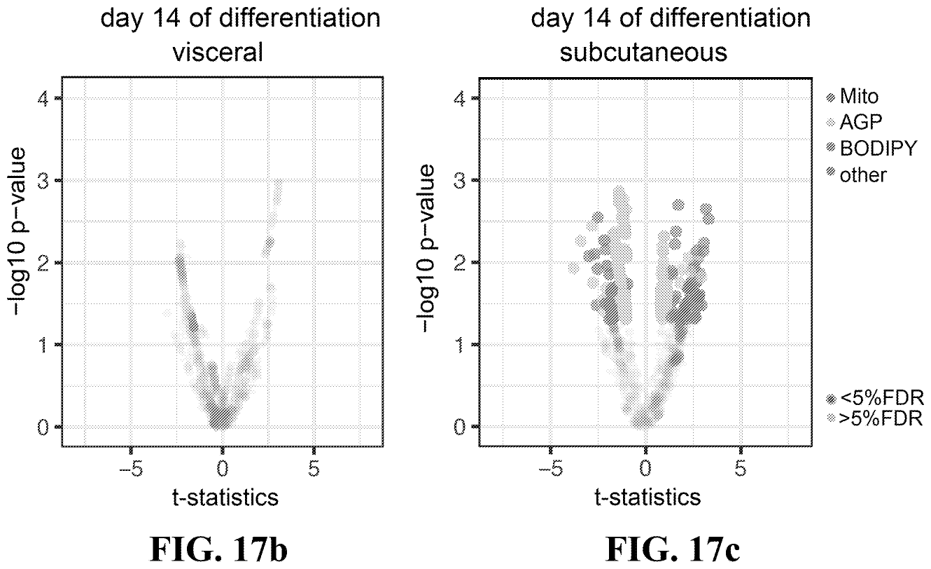

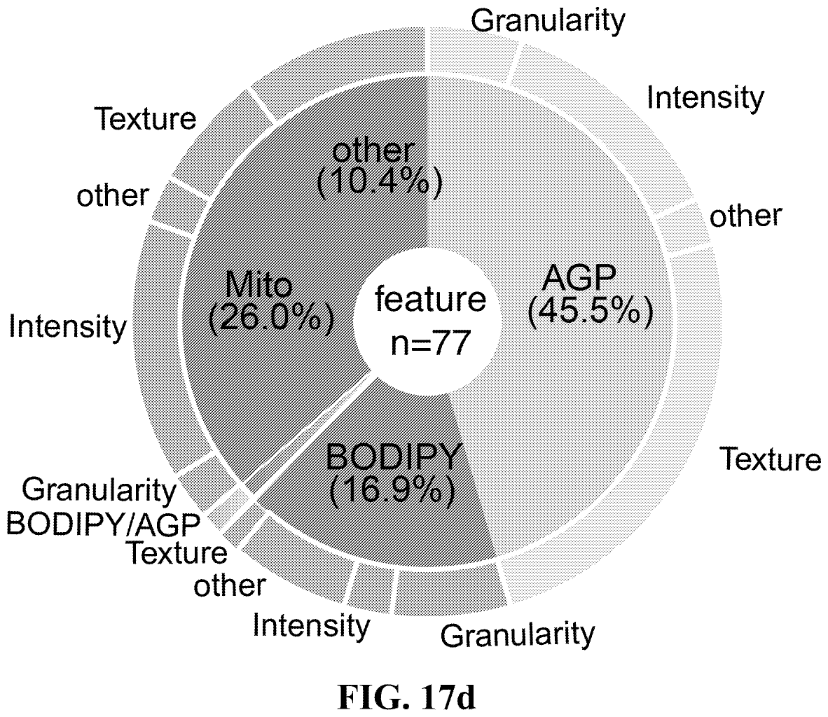

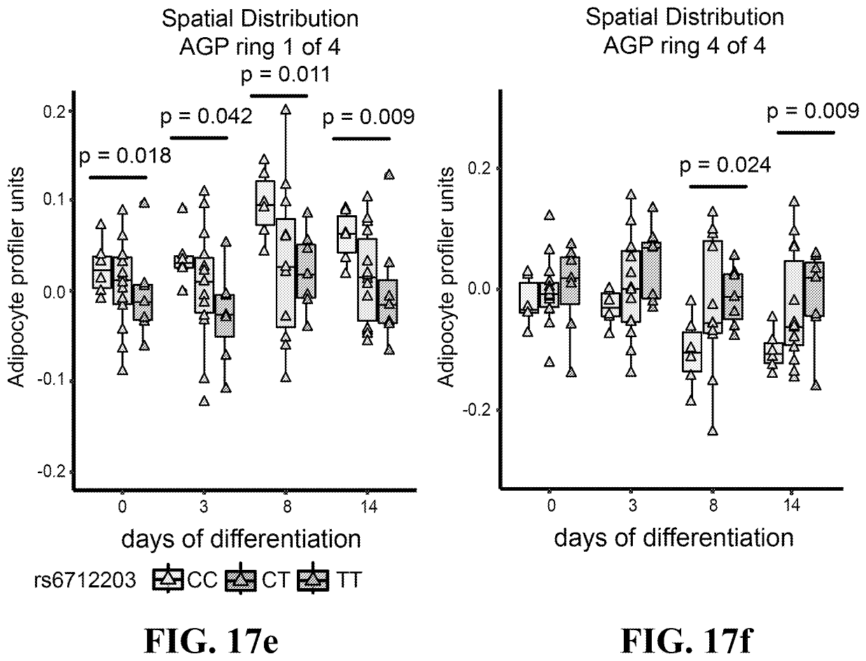

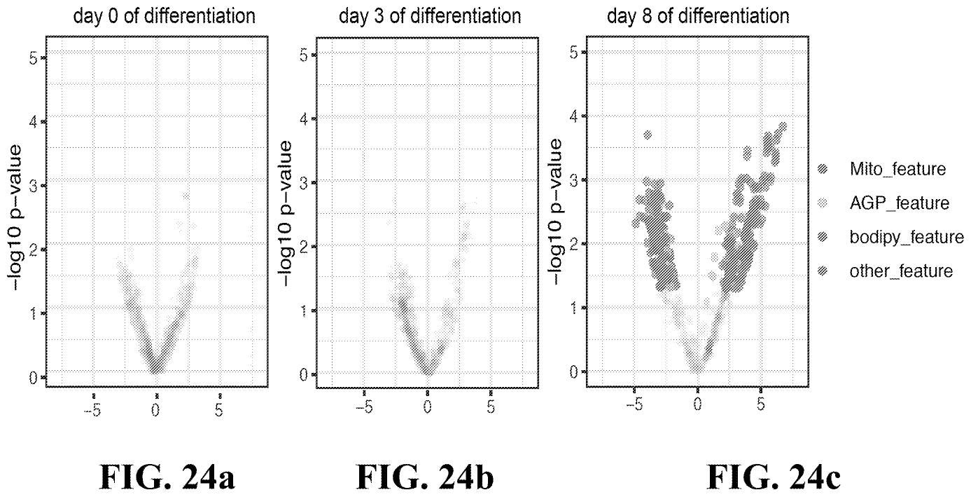

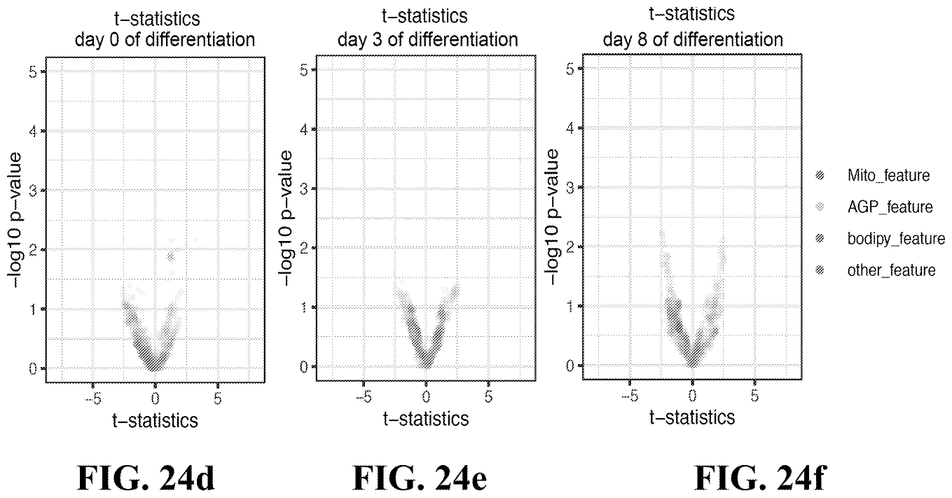

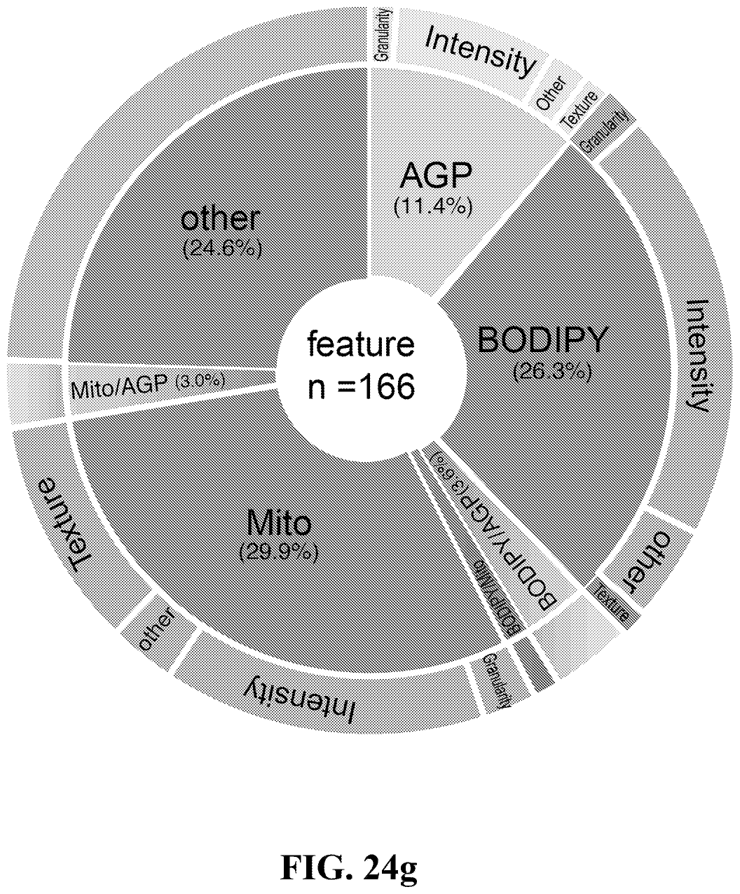

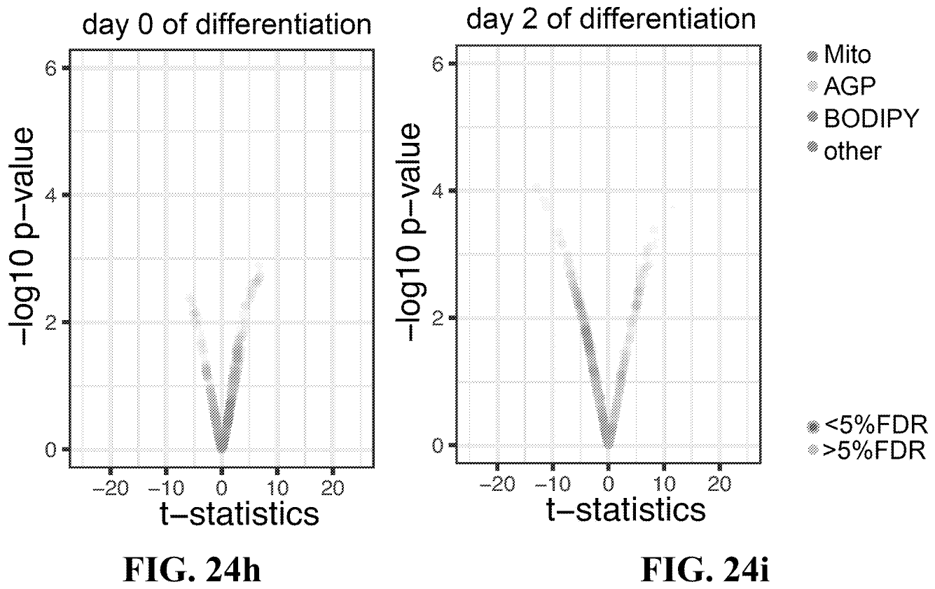

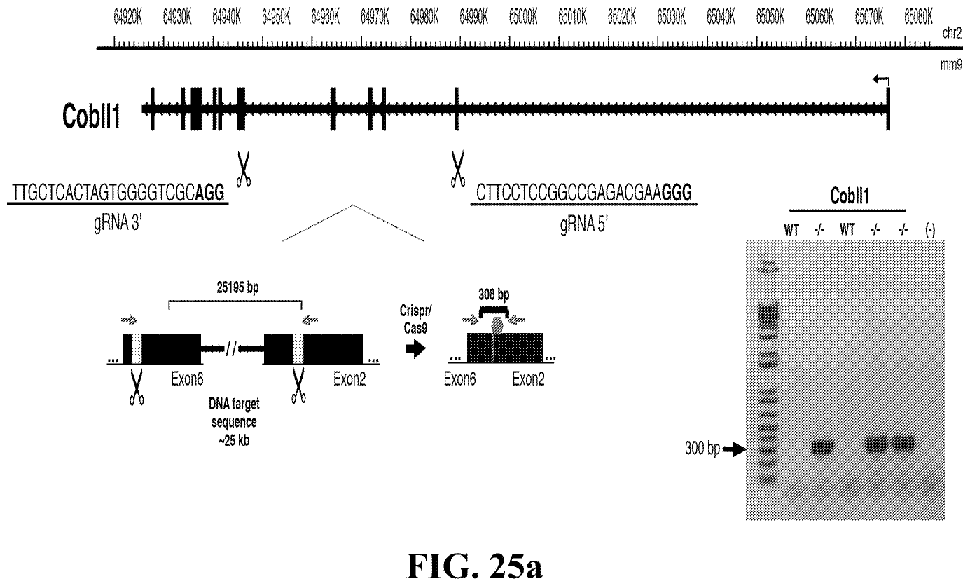

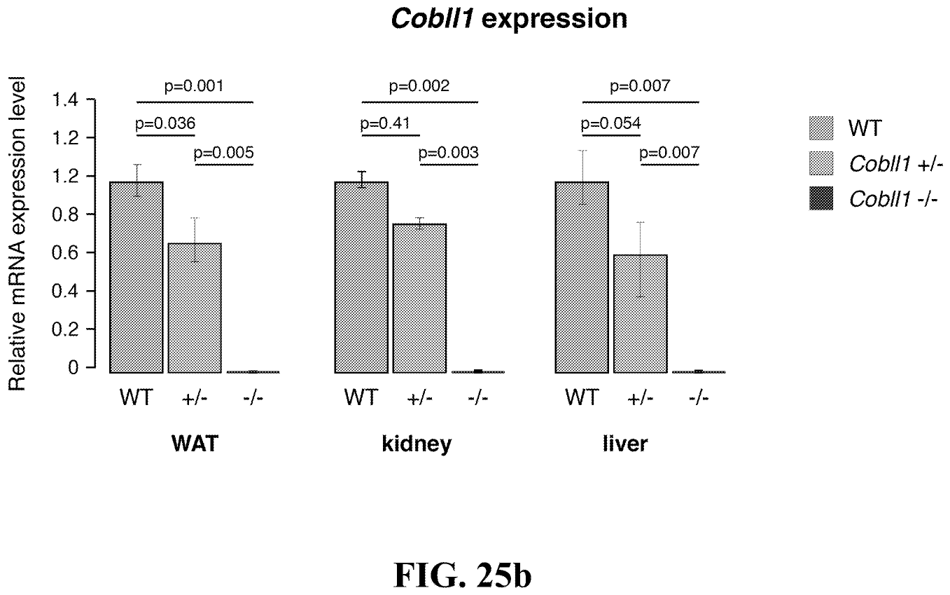

To confirm that the changes on the actin cytoskeleton and subsequent effects on adipocyte functions are under the genetic control of the rs6712203 MONW risk haplotype, Applicants used Adipocyte Profiler (see, Example 1) to phenotypically profile primary human adipocytes across differentiation from individuals carrying the risk haplotype (n=6) compared the non-risk haplotype (n=7) using Adipocyte Profiler (FIG. 17a). Applicants found that 77 morphological features, spread across BODIPY (16.9%), actin-associated AGP (45.5%) and mitochondrial (26.0%) channel, significantly differed between the haplotypes on day 14 of differentiation. (FDR 5%, Table 15). The data revealed that AGP and BODIPY features informative for the actin cytoskeleton and lipid accumulation differed in subcutaneous adipocytes from rs6712203 metabolic risk versus non-risk haplotype carriers (FIG. 17c-d, FIG. 24a-c). Applicants did not observe any significant difference in visceral adipocytes (FIG. 17b, FIG. 24d-f), consistent with the depot-specific effect of COBLL1 knockdown (FIG. 23n). Notably, Applicants found that the risk haplotype associates with increased actin-associated intensity in the center of the cell (day 0p=0.018, day 3 p=0.042, day 9 p=0.011 day 14 p=0.009, FIG. 17e) and decreased actin-associated intensity at the cell cortex (day 9 p=0.024 and day 14 p=0.009, FIG. 17f), which recapitulates the findings following COBLL1 knockdown and confirms that adipocytes from risk allele carriers are characterized by less cortical actin, required for insulin-stimulated glucose uptake in those cells and therefore directly relevant to fasting insulin levels and T2D. Applicants further observed that the risk haplotype associated with a difference in BODIPY objects count (day 8 p=0.043 and day 14 p=0.034, FIG. 17g), representative for number of lipid droplets, and higher BODIPY-related intensity (day 8 p 0.001, FIG. 17h), indicative for dysfunctional lipid droplet formation. These genetic effects on the actin cytoskeleton dynamics and lipid accumulation in AMSCs are fully coherent with the effects Applicants observed following COBLL1 knockdown experiments (FIG. 16b-f, h-i), suggesting that altered COBLL1 expression in the risk haplotype underlies the observed phenotypic effects in adipocytes. Together, these data show that the rs6712203 MONW risk locus, by altering COBLL1 expression levels, impacts actin remodeling in differentiating adipocytes, thereby dramatically affecting fat mass- and T2D-relevant cellular programs including adipocyte differentiation and lipid droplet formation and insulin-stimulated glucose uptake.

### Cobll1-Deficient Mice Display Metabolically Dysfunctional Lean Phenotype

Applicants generated a CRISPR engineered Cobll1 knockout (Cobll1−/−) mouse model to determine a potential role for Cobll1 in the regulation of metabolic function in vivo. First, Applicants sought to assess the effect of Cobll1 knockout on morphological and cellular profiles in differentiating murine perigonadal AMSCs by Adipocyte Profiler (day 0, day 2 and day 10 of differentiation, FIG. 18a). Applicants found that mostly BODIPY features significantly (<5% FDR) differ between knockout and control at day 10 of differentiation (FIG. 18b-c, FIG. 24h-i). More specifically the data revealed AMSCs of Cobll1 knockout mice show less lipid droplets (BODIPY_object_count p 0.0017, FIG. 18d), lower BODIPY Intensity (p=0.0073, FIG. 18e), higher BODIPY-related Granularity (p=0.0003, FIG. 18f) and in line with the BODIPY-related observation decreased actin-cytoskeleton related heterogeneity across Cytoplasm (Cytoplasm_Texture_Entropy_AGP, p=0.0200, FIG. 18g), indicating that Cobbl1 knockout in mice affects actin cytoskeleton remodeling and lipid accumulation during in vitro adipocyte differentiation, mimicking the observations in human adipocytes. Indeed, when examining the effect of Cobll1 knockout adipocytes on lipid accumulation using Oil-red-O, Applicants observed fewer differentiated adipocytes in Cobll1−/− compared to WT cells (FIG. 18h). Applicants also observed a significantly lower GPDH activity, an indicator of adipocyte differentiation of adipocytes, in Cobll1−/− mice was significantly lower compared to the WT littermates (P 0.004) (FIG. 18i), suggesting the ablation of Cobll1−/− leads to impaired adipogenesis, further supporting the finding in human adipocytes.

Applicants assessed the impact of the 2q24.3 MONW locus effector COBLL1 on organismal processes, assayed for growth and body composition phenotypes in Cobll1−/− mice. At 10 weeks of age, Applicants found that Cobll1−/− homozygous animals displayed 20-25% less weight gain compared to the WT control and Cobll1 heterozygous (Cobll1+/−) littermates (FIG. 18j-k), reflecting a significant reduction of the total fat mass percentage (3-5%), but with no difference in body length or in bone mineral density (BMD), suggesting that the lean phenotype of Cobll1−/− is due to reduced fat mass (FIG. 18l-n). Next, Applicants examined glucose homeostasis by performing Intraperitoneal glucose tolerance tests (IPGTT). Cobll1−/− mice displayed impairment glucose tolerance compared to WT and heterozygous littermates (FIG. 18o). In conclusion, the phenotypic characteristics of the Cobll1 knock-out mouse model recapitulate the MONW association patterns observed in humans and demonstrate how abrogation of Cobll1 links molecular and cellular phenotypes to organismal level metabolic phenotypes associated with genetic variation in the 2q24.3 locus in humans.

### Discussion—COBLL1

The 2q24.3 locus is pleiotropic in nature and, intriguingly, is associated with increased risk of T2D and simultaneously with decreased body fat percentage, reminiscent of a MOHN/MOH phenotype association signature. Here, Applicants applied a series of experimental and computational approaches to systematically dissect the 2q24.3 metabolic risk locus and link it to a causal variant (sr6712203), its effector gene (COBLL1), its causal cell type and cell context (developmental time point, adipose depot) and the cellular mechanisms the locus affects (actin remodeling). Together, these altered cellular functions that are relevant for T2D and body fat percentage and distribution, including adipocyte differentiation into metabolically active subcutaneous adipocytes, lipid metabolism and insulin-responsive glucose uptake. When ablating Cobll1 in mice Applicants show a ‘lipodystrophy-like phenotype’, recapitulating the pleiotropic association with T2D and decreased body fat mass in humans. These data use genetic evidence to provide mechanistic evidence that a common genetic variant limits peripheral energy storage capacity and simultaneously affects insulin responsiveness.

The results of this study lend support to the common hypothesis that the individual risk of T2D and fasting insulin is modified by changes to the mass, distribution and function of adipose tissue (Lotta et al. 2017; Small et al. 2018), and that a metabolically healthy state is largely dependent on subcutaneous adipose tissue expandability. Inherited and acquired lipodystrophies, as characterized by the selective or global perturbation of adipose tissue function, mass and distribution, result in severe forms of insulin resistance and diabetes, and shared molecular mechanisms between rare familial partial lipodystrophy type 1 and common forms of insulin resistance at the genetic level have been previously suggested (Lotta et al. 2017). Several common metabolic risk loci are characterized by a MONW/MOH association, and distinct association signatures suggest multiple mechanisms at play, most of which remain to be identified (Loos and Kilpeläinen 2018; Kilpeläinen et al. 2011; Fathzadeh et al. 2020). Previous work has convincingly implicated variants at the FAM13A locus to affect metabolic disease risk by affecting subcutaneous adipocyte differentiation (Fathzadeh et al. 2020). In this work, Applicants implicate for the first time actin cytoskeleton remodeling as a critical factor for subcutaneous adipocyte function and as causally involved in metabolic disease progression in humans, stressing the notion that MONW/MOH predisposing loci control distinct cellular programs.

Applicants observed evidence of sex-dimorphic effects when conditioning MONW traits on rs6712203 which is in line with a reported sexual dimorphism for WHIR consistently conveying stronger effects in women (Heid et al. 2010; Morris et al. 2012; Randall et al. 2013; Sung et al. 2016) and a sex-independent effect on T2D (Vujkovic et al. 2020; Spracklen et al. 2020) and with a sex-dimorphic effect on gene expression for COBLL1, but not for GRB14 (Lagou et al. 2021).

The COBLL1 protein has been introduced as a biomarker of high prognostic value for different types of cancer (Gordon et al., 2003, 2009; Wang et al., 2013; Han et al., 2017), a modulator of cell morphology in prostate cancer (Takayama et al. 2018), and lipid metabolism and insulin signaling in adipocytes (Chen et al. 2020). Here, Applicants establish a chain-of-causation linking the 2q24.3 locus to its functional variant, its adipocyte cell type and context specific effect, its regulatory element, its effector gene COBLL1, and finally its causal cellular function, i.e. actin remodeling in differentiating adipocytes, which is under the genetic control of both the locus and the target gene. Consequently, Applicants establish the gene as a key regulator of subcutaneous adipocyte differentiation, lipid metabolism and insulin sensitivity at the cellular as well as the organismal level. These findings are in line with recent reports linking actin dynamics, regulated by the F/G-actin ratio, and insulin-stimulated trafficking and fusion of GLUT4 vesicles (Chen et al. 2018; Kanzaki and Pessin 2001; Kim et al. 2019).

While the insulin receptor adaptor protein GRB14 (Growth Factor Receptor Bound Protein 14) is an intuitive effector target gene at the 2q24.3 locus and has been shown to affect glucose tolerance (Cariou et al. 2004; Cooney et al. 2004; Chen et al. 2020), Applicants causally implicate COBLL1 as at least one causal effector gene at the locus. Applicants note that COBLL1 as the effector gene that underlies the T2D) association is further corroborated by the T2D-associated coding variant Asn939Asp in COBLL1 (MAF=0.12, p=4.7×10⁻¹¹) (Fuchsberger et al. 2016). Furthermore, recent rare variant aggregation analyses at COBLL1 revealed nominal association with WHR (Kan et al. 2016), concordant with the findings that COBLL1 drives at least part of the 2q24.3 genetic risk. the sequence based predictive models score rs6712203 highest across all 2q24.3 haplotype variants though multiple other variants at the locus are predicted to affect regulatory activity as well. Therefore, while beyond the scope of this study, Applicants note that multiple variants could act in concert at this locus potentially implicating GRB14 along with COBLL1 as effector genes.

The 2q24.3 locus is a prime example of a common genetic locus that predisposes to limited peripheral adipose storage capacity and insulin resistance, driven by an impairment of dynamic actin cytoskeleton remodeling process of the differentiating subcutaneous adipocyte.

### Methods—COBLL1

### Human Primary AMSCs Isolation and Differentiation

Applicants obtained AMSCs from subcutaneous and visceral adipose tissue from patients undergoing a range of abdominal laparoscopic surgeries (sleeve gastrectomy, fundoplication or appendectomy). The visceral adipose tissue is derived from the proximity of the angle of His and subcutaneous adipose tissue obtained from beneath the skin at the site of surgical incision. Additionally, human liposuction material was obtained from a collaborating private plastic surgery clinic Medaesthetic Privatklinik Hoffmann & Hoffmann in Munich, Germany. Isolation of AMSCs was performed as previously described (Claussnitzer 2014; Hauner et al. 2001).

### Differentiation of Human AMSCs

For imaging, cells were seeded at 10K cells/well in 96-well plates (High-content imaging; Cell Carrier, Perkin Elmer #6005550) or seeded at 18,000 cells/well in collagen IV coated 8 well μ-slides (Higher-resolution imaging; ibidi, Gräfelfing, Germany #/80822) and induced 4 days after seeding. For RNAseq, cells were seeded at 40K cells/well in 12-well dishes (Corning). Before Induction cells were cultured in proliferation medium (Basic medium consisting of DMEM-F12 1% Penicillin-Streptomycin, 33 μM Biotin and 17 μM Pantothenate supplemented with 0.13 μM Insulin, 0.01 ug/ml EGF, 0.001 ug/ml FGF, 2.5% FCS). Adipogenic differentiation was induced by changing culture medium to induction medium. (Basic medium supplemented with 0.861 μM Insulin, 1 nM T3, 0.1 μM Cortisol, 0.01 mg/ml Transferrin, 1 μM Rosiglitazone, 25 nM Dexamethasone, 2.5 nM IBMX). On day 3 of adipogenic differentiation culture medium was changed to differentiation medium (Basic medium supplemented with 0.861 μM Insulin, 1 nM T3, 0.1 μM Cortisol, 0.01 mg/ml Transferrin). Medium was changed every 3 days. Visceral-derived AMSCs were differentiated by further adding 2% FBS as well as 0.1 mM oleic and linoleic acid to the induction and differentiation media.

### Genotyping

Genotyping was performed using the Illumina Global Screening beadchip array. DNA was extracted using Qiagen DNeasy Blood and Tissue Kit (Qiagen 69504) and sent to the Oxford Genotyping Center for genotyping on the Infinium HTS assay on Global Screening Array bead-chips. Genotype QC was done using GenomeStudio and genotypes were converted into PLINK format for downstream analysis. Applicants checked sample missingness but found no sample with missingness >5%. For the remaining sample quality control (QC) steps, Applicants reduced the genotyping data down to a set of high-quality SNPs. These SNPs were: (a) Common (minor allele frequency >10%); (b) Had missingness <0.1%; (c) Independent, pruned at a linkage disequilibrium (R²) threshold of 0.2; (d) Autosomal only; (e) Outside the lactase locus (chr2), the major histocompatibility complex (MHC, chr6), and outside the inversions on chr8 and chr17; (f) In Hardy-Weinberg equilibrium (P>1x10 3). Using the remaining ˜65,000 SNPs, Applicants checked samples for inbreeding (--het in PLINK), but found no samples with excess homozygosity or heterozygosity (no sample >6 standard deviations from the mean). Applicants also checked for relatedness (--genome in PLINK) and found one pair of samples to be identical; Applicants kept the sample with the higher overall genotyping rate. Finally, Applicants performed PCA using EIGENSTRAT and projected the samples onto data from HapMap3, which includes samples from 11 global populations. Six samples appeared to have some amount of non-European ancestral background, while the majority of samples appeared to be of European descent. Applicants removed no samples at this step, selecting to adjust for principal components in genome-wide testing. However, adjustment for principal components failed to eliminate population stratification, and Applicants therefore restricted to samples of European descent only, defined as samples falling within +/−10 standard deviations of the first and second principal component values of the CEU (Northern and Western European-ancestry samples living in Utah) and TSI (Tuscans in Italy) samples included in the HapMap 3 dataset. Finally, sex information was received after initial sample QC was complete. As a result, one sample with potentially mismatching sex information (comparing genotypes and phenotype information) was discovered after analyses were complete and therefore remained in the analysis.

### SNP Quality Control.

Applicants removed all SNPs with missingness >5% and out of HWE, P<1x10-6. Applicants also removed monomorphic SNPs. Finally, Applicants set heterozygous haploid sites to missing to enable downstream imputation. The final cleaned dataset included 190 samples and ˜700,000 SNPs. Applicants note that histology data was not available for all genotyped samples.

### Genotype Imputation.

For the genotyped cohorts without imputation data (ENDOX and MOBB) Applicants performed imputation via the Michigan Imputation Server. Applicants aligned SNPs to the positive strand, and then uploaded the data (in VCF format) to the server. Applicants imputed the data with the Haplotype Reference Consortium (IIRC) panel, to be consistent with the fatDIVA data which was already imputed with the IRC panel. Applicants selected EAGLE as the phasing tool to phase the data. To impute chromosome X, Applicants followed the server protocol for imputing this chromosome (including using SHAPEIT to perform the phasing step).

### ATAC-seq in Immortalized AMSCs

ATAC-seq was performed by adapting the protocol from (Buenrostro et al., 2015) by adding a nuclei preparation step. Differentiating cells were lysed directly in cell culture plate at four time-points during differentiation (before adipogenesis was induced (DO), during early (D3) and advanced differentiation (D6), as well as at terminal differentiation (D24)). Ice-cold lysis buffer was added directly onto cells grown in a 12-well plate. Plates were incubated on ice for 10 minutes until cells were permeabilized and nuclei released. Cells in lysis buffer were gently scraped off the well and transferred into a chilled 1.5 ml tube to create crude nuclei. Nuclei were spun down at 600×g for 10 minutes at 4° C. Nuclei pellets were then re-suspended in 40 μl Tagmentation DNA (TD) Buffer (Nextera, FC-121-1031) and quality of nuclei assessed using trypan blue. Volume of 50,000 nuclei was determined using a haemocytometer. Transposition reaction was performed as previously described (Buenrostro et al., 2015). All tagmented DNA was PCR amplified for 8 cycles using the following PCR conditions: 72° C. for 5 minutes, 98° C. for 30 seconds, followed by thermocycling at 98° C. for 10 seconds, 63° C. for 30 seconds and 72° C. for 1 minute. Quality of ATAC-seq libraries was assessed using a Bioanalyzer High Sensitivity ChIP (Applied Biosystems). The profiles showed that all libraries had a mean fragment size of ˜200 bp and characteristic nucleosome patterning, indicating good quality. Libraries were pooled and sequenced on a HiSeq4000 Illumina, generating 50 mio reads/sample, 75 bp paired end. To reduce bias due to PCR amplification of libraries, duplicate reads were removed. Sequencing reads were aligned to hs37d5 and BWA-MEM was used for mapping. All experiments were performed in technical duplicates.

### Adipocyte Painting in Human and Mouse AMSCs

Human primary AMSCs and mouse AMSCs were plated and differentiated in 96-well CellCarrier plates (Perkinelmer #6005550) for 14 days for high content imaging at day 0, day 3, day 8 and day 14 of adipogenic differentiation. On the respective day of the assay, cell culture media was removed and replaced by 0.5 μM Mitotracker staining solution (1 mM MitoTracker Deep Red stock (Invitrogen #M22426) diluted in culture media) to each well followed by 30 minutes incubation at 37° C. protected from light. After 30 min Mitotracker staining solution was removed and cells were washed twice with Dulbecco's Phosphate-Buffered Saline (1×), DPBS (Corning®) #21-030-CV) and 2.9 μM BODIPY staining solution (3.8 mM BODIPY 505/515 stock (Thermofisher #D3921) diluted in DPBS) was added followed by 15 minutes incubation at 37° C. protected from light. Subsequently, cells were fixed by adding 16% Methanol-free Paraformaldehyde, PFA (Electron Microscopy Sciences #15710-S) directly to the BODIPY staining solution to a final concentration of 3.2% and incubated for 20 minutes at RT protected from light. PFA was removed and cells were washed once with Hank's Balanced Salt Solution (1×), HBSS (Gibco #14025076). To permeabilize cells 0.1% Triton X-100 (Sigma Aldrich #X100) was added and incubated at RT for 10 minutes protected from light. After Permeabilization multi-stain solution (10 units of Alexa Fluor™ 568 Phalloidin (ThermoFisher #/A12380), 0.01 mg/ml Hoechst 33342 (Invitrogen #/H3570), 0.0015 mg/ml Wheat Germ Agglutinin, Alexa Fluor™ 555 Conjugate (ThermoFisher #W32464), 3 μM SYTO™ 14 Green Fluorescent Nucleic Acid Stain (Invitrogen/S7576) diluted in HBSS) was added and cells were incubated at RT for 10 minutes protected from light. Finally, staining solution was removed and cells were washed three times with HBSS. Cells were imaged using a Opera Phenix High content screening system. Per well Applicants imaged 25 fields.

### Staining and Microscopy

To stain the actin cytoskeleton, COBLL1 and nuclei, cells were washed twice with ice cold PBS and fixed with paraformaldehyde Roti-Histofix 4% (Roth, Karlsruhe, Germany) for 15 min. Cells were washed twice with ice cold PBS for 5 min and incubated with ice cold 0.1% Triton X/PBS (Roth, Karlsruhe, Germany) for 5 min. Cells were washed again twice with PBS and incubated for 1 hour at RT with 4% BSA, then incubated with 1:100 primary COBLL1-antibody (specification: HPA053344; atlas antibodies, Bromma, Sweden) overnight at 4° C., followed by one hour at room temperature. Cells were washed twice with PBS and stained with 0.46% Bisbenzimide H 33258 (Sigma-Aldrich, Steinheim, Germany), 1% Phalloidin-Atto-565 (Sigma-Aldrich, Steinheim, Germany) and the secondary antibody against COBLL1 1:200 Alexa-Fluor 488 (Abcam, Cambridge, UK). Cells were incubated for one hour at RT in the dark. Afterwards, cells were washed twice with PBS for 5 min and kept in PBS at 4° C. until imaging. Images were acquired on a Leica DMi8 microscope using the HIC PL APO ×63/1.40 oil objective. Images were processed using the Leica LasX software.

### Adipocyte Profiler

Quantitation was performed using CellProfiler 3.1.9. Prior to processing, flat field illumination correction was performed using functions generated from the mean intensity across each plate. Nuclei were identified using the DAPI stain and then expanded to identify whole cells using the AGP and Bodipy stains. Regions of cytoplasm were then determined by removing the Nuclei from the Cell segmentations. Speckles of Bodipy staining were enhanced to assist in detection of small and large individual Bodipy objects. For each object set measurements were collected representing size, shape, intensity, granularity, texture, colocalization and distance to neighboring objects. After feature extraction data was filtered by applying automated and manual quality control steps. First, fields with a total cell count less than 50 cells were removed. Second, fields that are corrupted by experimental induced technical artifacts were removed by applying a manually defined quality control mask. Furthermore, blocklisted features that are known to be noisy and generally unreliable were removed. After filtering data were normalised per plate using a robust scaling approach that subtracts the median from each variable and divides it by the interquartile range. For each individual wells were aggregated for downstream analysis by cell depot and day of differentiation. Subsequent data analyses were performed in R3.6.1 using base packages unless noted. For dimensionality reduction visualization Uniform manifold approximation and projection maps (UMAP) were created using the UMAP R package (github.com/Imcinnes/umap) with default settings.

To test whether there is a difference of morphological profiles at any day of differentiation due to COBLL1 KD both individuals were analyzed separately using a t-test. To test whether there is a difference of morphological profiles at any day of differentiation between risk and non-risk haplotype a multi-way analysis of variance (ANOVA) was performed. Differences in morphological profiles between TT (n=7) and CC (n=6) allele carriers were adjusted for sex, age, BMI and batch. To overcome multiple testing burden p-values were corrected using false positive rate (FDR) described in R package “qvalue” (github.com/StoreyLab/qvalue). Features with FDR <5% were classified to be significant and filtered based on redundancy and effect size.

COBLL1 Silencing Using siRNA

All silencing experiments were performed on 4 technical replicates. One day before silencing, AMSCs were plated into 96-well plates with 10K cells/well or collagen IV coated 8 well glass μ-slides with 18K cells/well using growth medium. RNA-based silencing of COBLL1 was performed using RNAiMAX Reagent (ThermoFisher #13778075) and following the manufacturer's protocol. Briefly, Lipofectamine® RNAiMAX Reagent was diluted in Opti-MEM medium (Gibco, Cat #11058021). At the same time, siRNA was diluted in Opti-MEM medium. Then, diluted siRNA was added to the diluted Lipofectamine®) RNAiMAX reagent at a ratio 1:1 and incubated for 5 min. For coated 8 well glass μ-slides incubated for 20 min at RT. The concentration of reagents per well in a 96-well plate were 0.5 μl (10 μM) of silencing oligo (Ambion Cat #4392420, IDs22467) or negative control duplex (Ambion Cat #4390846), and 1.5 μl of lipofectamine RNAiMAX Reagent. The plate was gently swirled and placed in a 37° C.′ incubator at 5% CO2 for three days. Cells were then induced to differentiate following the standard differentiation cocktail or harvested for gene expression analysis to assess knockdown efficiency.

RNA Preparation and qPCR

Total RNA was extracted with Trizol (Ambion 15596026) and the Direct-zol RNA MiniPrep Kit (Zymo R2052) following the manufacturer's instructions. cDNA was synthesized with High-Capacity cDNA Reverse Transcription Kit (Applied Biosystems 4368814) following the manufacturer's instructions. qPCR was performed using Thermo Scientific PCR Master Mix (Thermo Scientific K0172) and taqman probes for target gene COBLL1 (Thermo Scientific, Cat #4448892, ID Hs01117513_m1) and housekeeping gene CANX (Thermo Scientific, Cat #/4448892, ID Hs01558409_m1). Relative gene expression was calculated by the delta delta Ct method. Target gene expression was normalized to expression of CANX.

### RNAseq and Splicing Analysis

RNA-seq reads were trimmed using SeqPurge with the following command:

SeqPurge -a1
(SEQ ID NO: 55)
CTGTCTCTTATACACATCTCCGAGCCCACGAGAC
-a2
(SEQ ID NO: 56)
CTGTCTCTTATACACATCTGACGCTGCCGACGA

For transcript-level quantification, trimmed reads were analysed using Kallisto (with 25 bootstraps) and the TPM estimates were log-transformed and the top 10 PCs were computed. Next, reads were summed across all transcripts of a given gene to obtain gene-level estimates of the expression in each sample.

For splicing analysis with Leafcutter, reads were mapped with STAR using the following arguments: STAR --twopassMode Basic --outSAMstrandField intronMotif --readFilesCommand zcat --outSAMtype BAM Unsorted

And then processed using samtools and regtools to convert to a junc file with the following command: regtools junctions extract -s 1 -a 8 -m 50 -M 500000

Finally, reads were clustered into splicing events with the following command from the Leafcutter project: leafcutter_cluster_regtools.py -j <files> -m 50 -l 500000

These clusters were then converted to transcripts per million and modeled as a function of rs6712203 genotype.

### RNA Pathway Enrichment Analysis

Transcript-level (log) RNA expression was compared between COBLL1 and all other quantified genes using linear regression. The effect of COBLL1 on other genes was compared adjusted for expression PCs (described above), sample depot source, cell line, and day of differentiation. This resulted in effect sizes of individual genes in terms of how similar they are to COBLL1 and those with estimates that had Bonferroni adjusted P-value >1e-3, absolute effect size <0.1 or >10 were excluded. This left a list of similarly expressed genes with strong association with COBLL1, which were uploaded to Enrichr and analysed as a gene list against the KEGG, WikiPathways, and HCI pathways. The full set of tests is available at maayanlab.cloud/Enrichr/enrich?dataset 1a9a07019bfd8bbddc6cb6c26641bfcf and the sensitivity evaluation in which very lowly expressed and highly expressed genes were not excluded (via the thresholds on absolute effect size described above) are available at maayanlab.cloud/Enrichr/enrich?dataset 231b12708d04818007d93364c489fab7.

### PMCA Variant Conservation Analysis

PMCA results were replicated from (Claussnitzer et al., 2014). Briefly, transcription factor binding sites and their co-occurrence across species were tallied and classified into complex and non-complex regions. Complex regions were counted on the basis of motifs aligned across species, and those were then plotted against the Basset scores (below) to discover putative causal variants.

### Basset Variant Effect Prediction Analysis

Basset models were trained and evaluated as in (Sinnott-Armstrong et al. 2021). Briefly, models were trained to capture chromatin regulation relevant to adipocyte differentiation and these effects were estimated by determining the difference in effect between alleles at each variant. The variants with the largest effect on accessibility were considered the most important and most likely to be causal.

### Allele-Specific Accessibility Analysis

Allele-specific analyses were performed as in (Sinnott-Armstrong et al. 2021). Briefly, reads were aligned from a heterozygous individual on the basis of the variant and the number of reads supporting each allele were tallied at each timepoint and across variants on the haplotype.

### Conditional and BMI-Dependent Variant Association Analysis

Variants (n=6167) within 100 kb of rs6712203 were included in the analysis. White British individuals in the UK Biobank were analyzed with phenotypes type 2 diabetes (as described in (Eastwood et al. 2016)), log waist-to-hip ratio adjusted for body mass index, hip circumference, and whole body fat mass. Individuals were stratified on the basis of reported sex and filtered to the White British unrelated individuals as described in (Sinnott-Armstrong et al. 2021). Conditional analyses and all associations were performed using Plink2.

### Electrophoretic Mobility Shift Assay (EMSA)

EMSA experiments were performed using double stranded Cy5-labelled probes with the risk or non-risk allele of each variant at mid-position. The forward Cy5-labelled strand (for rs6712203 5′-TTAATTTGCCTCATTCATCA[C/T]ATGCAATTCTGGCAAGGAA-3′ (SEQ ID NOS: 57-58) and for rs10195252 5′-CCCCACTTCCCTCTAGGGAA[T/C]GGGAAAGAACATTTAACCT-3′ (SEQ ID NOS; 59-60) and respective unlabeled reverse complementary strands were synthesized (Eurofins, Ebersberg, Germany), annealed and purified from single stranded remains by excision from a 12% polyacrylamide gel. Nuclear protein extracts from primary mature human adipocytes were extracted according to the protocol described by Dugail and colleagues (Dugail 2001).

For EMSA experiments, 1-2 μl buffer containing 3-5 ug proteins was added to 10 mM TrisHCl (pH 7.5), 1 mM MgCl2, 50 mM NaCl, 0.5 mM EDTA, 4% (v/v) glycerol, 0.5 mM DTT and 30 ng/μl poly(dI-dC). After 10 minutes incubation on 4° C., 1 ng of the respective Cy-5 labelled probe was added and the samples were incubated for 20 min at 4° C. After addition of loading buffer with 25 mM TrisHCl pH 7.5, 0.02% OrangeG, 4% glycerol, the samples were subjected onto a nondenaturing 5.3% polyacrylamide gel. After gel separation, Cy-5 fluorescence was detected using the Typhoon TRIO I imager (GE Healthcare, Germany).

### POU2F2 Affinity Modeling Using the Intragenomic Replicates (IGR) Method

The Intragenomic Replicates (IGR) method was used for POU2F2 affinity modeling using POU2F2 ChIP-seq data as previously described (Cowper-Sal lari et al. 2012). In order to correct for systematic bias in the sequencing depth around particular k-mers, all scores were offset by a “baseline” value, defined as the average signal between the forward and reverse complement instances of the k-mer between −200 and −195 and between 195 and 199 bases away from the k-mer center. Thus, if the value across the whole −200 to +199 context was approximately equal, then the overall score is approximately zero, and positive estimated affinities are only possible in cases where the score in the middle of the context is significantly higher than the outside. To further include only large effect binding differences, the “prominence” was defined as the maximum score across any point in the context for either the forward or reverse complement version of the k-mer for both alleles and the “maximum difference” as the maximum absolute difference in scores between the two alleles at any point in the window. The “baseline ratio” was defined as the ratio of the maximum difference to the prominence, which varies between 0 (if the two alleles are equal at all points) and 2 (if they are perfectly complementary at their highest absolute point).

In order to find only high-quality putative disrupted binding sites, the k-mer sequence that gave the highest affinity under the germline was recorded as “reference” and the k-mer sequence which gives the highest affinity under the somatic variant as “alternate.” The “quality” of a given kmer was defined as the correlation between the average context plot forward and the reverse of the average context plot of the reverse complement, and the “symmetry” of a given k-mer as the correlation between the average context plot forward and the average context plot reverse. Quality is high when the antiparallel binding is preserved and symmetry is high when the peak signal is centered with respect to the variant. The results were included as “passed” when the Bonferroni corrected p-value for the comparison is less than 0.05, the baseline ratio is greater than 0.5, the quality and symmetry are both greater than 0.85 for one of the alleles, and the quality and symmetry are both greater than 0.5 for the other allele.

### Microarray Expression Data

A global gene expression measurement was performed, using Illumina HumanRef-8 v.3 BeadChip microarrays from whole abdominal subcutaneous adipose tissue. Signal intensities were quantile normalized before the correlation analysis.

### SGBS Genome Editing

To edit the rs6712203 heterozygous allele in SGBS preadipocytes to the homozygous risk (CC) and non-risk (TT) alleles Applicants applied the CRISPR/Cas9 homology directed repair genome editing approach. The hCas9 vector was purchased from Addgene (Plasmid ID #41815). The guide sequence was selected using the design tool (Zhang Lab, MIT 2013) with a predicted number of 228 potential off target sites, located 211 bp upstream of rs6712203. It was cloned in front of the U6 promoter into the BbsI cloning site of the sgRNA-expression vector (Dr. Ralf Kühn, Helmholtz Zentrum München-Neuherberg), using double stranded oligonucleotides 5′-CACCGACTCTCCACTACCATTGCCA-3′ (SEQ ID NO: 61) and 5′-AAACTGGCAATGGTAGTGGAGAGTC-3′ (SEQ ID NO: 62). For amplification of the 2009 bp homology region with the risk or non-risk allele of rs6712203 at mid position, genomic DNA of SGBS cells was amplified with primers 5′-GGTGGTCCCATTAAAAAGAAAGAAGCTTGG-3′ (SEQ ID NO: 63) and 5′-CTTCTCTTTTACCCTGCTGGCTACTGGTTG-3′ (SEQ ID NO: 64) using the High-Fidelity Q5 DNA polymerase (NEB). The gel purified PCR product was cloned into the blunt end pJet1.2 vector using the CloneJET PCR Cloning kit (Fermentas). A clone with the rs6712203 C allele was selected and the corresponding T allele vector was generated using the Q5 Site-Directed Mutagenesis Kit (NEB) with the primers 5′-TCATTCATCATATGCAATTCTGG-3′ (SEQ ID NO: 65) and 5′-GGCAAATTAATATTTAGGATTATATC-3′ (SEQ ID NO: 66). To avoid Cas9 reactivity after genome editing, the NGG guide target sequence was mutated to NCG in both homology vectors with the primers 5′-CCATTGCCAACGGCTGAGTCAG-3′ (SEQ ID NO: 67) and 5′-TAGTGGAGAGTTCTCACAAAAC-3′ (SEQ ID NO: 68). SGBS cells were co-transfected with the GFP (Lonza), the hCas9, the respective sgRNA, and the pMACS 4.1 (Milteny) plasmids using the Amaxa-Nucleofector device (program U-033) (Lonza). The cells were sorted using the MACSelect™ Transfected Cell Selection kit (Miltenyi). The integrity of each edited vector construct and the SGBS cell nucleotide exchange was confirmed by DNA sequencing (Eurofins, Ebersberg, Germany).

### Lentiviral SGBS Cell Transduction

For the production of lentiviral particles, the MISSON® Lentiviral Packaging Mix (Sigma Aldrich, Steinheim, Germany) was used according to the manufacturer's instructions. Briefly, packaging cells HEK293T were grown in a low antibiotic growth medium (DMEM, 10% FCS, 0.1% penicillin/streptomycin). When cells were about 70% confluent they were co-transfected, using X-treme GENE HP (Roche, Penzberg, Germany), with the packaging plasmid pCMVdeltaR8.91, the envelope plasmid pMD2.G and the pLKO-based plasmid containing shRNA against the human target gene COBLL1 NM_014900.2-3071s1c1, COBLL1 NM_014900.2-4440s1c1, GRB14 NM_004490.1-1581s1c1 or empty-vector MISSION® TRC2 pLKO.5-puro plasmid (Sigma Aldrich, Steinheim, Germany). The cells were incubated for 24 hours, the medium was discarded and replaced with a serum rich medium (30% FCS). The supernatant containing the viable virus particles was collected 48 and 72 hours post transfection, centrifuged to remove cellular debris, and stored at −80° C.

SGBS cells were seeded at a concentration of 2.6×10⁴ cells per 6-well plate and grown in normal growth medium. After 24 hours the medium was replaced and supplemented with 8 μg/ml Polybrene (Sigma-Aldrich, Steinheim, Germany) and virus supernatant with a multiplicity of infection (MOI) of 2. On the consecutive 2 days cells were washed with PBS and medium was replaced to remove the virus. The medium was supplemented with 0.5 ug/ml puromycin 96 hours after infection, to select stable clones. When cells were grown confluent, puromycin was removed from the medium and the cells were differentiated until day 16. Target gene silencing was confirmed after selection and on the day of each experiment by qRT-PCR.

### Glycerol-3-Phosphate Dehydrogenase (GPDH) Activity Measurement

Cells were grown to confluence and differentiated until day 16 in 6 well plates. Cells were collected in a GPDH buffer with 0.05 M Tris/HCl (pH 7.4), 1 mM EDTA and 1 mM Mercaptoethanol, before they were stored at −80° C. until further use. Samples were gently defrosted on 4° C.′, and were sonified for 7 sec at 29% and centrifuged for 10 min at 10.000 g on 4° C. GPDH activity was measured as previously described (Pairault and Green 1979). Briefly, GPDH activity was assessed, measuring the conversion of dihydroxyacetone phosphate (DHAP) (Sigma, St. Louis, USA), in the presence of the coenzyme nicotinamide adenine dinucleotide (NADH) (Omnilab-Applichem, Bremen, Germany) at a wavelength of 340 nm, using the Tecan Infinite 200 (Tecan, Crailsheim, Germany). Protein concentrations were assessed using the BCA-RAC protein assay kit (Thermo Scientific, Germany), with BSA standard samples in GPDH buffer for quantification. The value for each condition was calculated using the ratio between GPDH activity and protein concentration.

### Glucose Uptake, Lipolysis and Western Blot Analysis

For glucose, glycerol and Western Blot analysis shRNA COBLL1 and shRNA empty-vector SGBS cells were differentiated until day 16 in 6 well plates. The insulin-stimulated 2-desoxy-D)-glucose (2-DG) uptake experiment was performed as previously described (Claussnitzer et al. 2011). Briefly cells were incubated in glucose-free DMEM and F12 (1:1) containing 1% penicillin/streptomycin, 16 μM biotin, 36 μM pantothenic acid, 14.3 mM NaHCO3 and 0.5 mM Na-pyruvate (Sigma-Aldrich, Steinheim, Germany) for 12 hours. The medium was replaced with 118 mM NaCl, 1.2 mM KH2PO4, 4.8 mM KCl, 1.2 mM MgSO4, 2.5 mM CaCl2, 10 mM HEPES, 2.5 mM Na-pyruvate (Sigma-Aldrich, Steinheim, Germany), 0.5% BSA (Sigma-Aldrich, Steinheim, Germany) (pH 7.35). After 1.5 hours the same buffer was added fresh either without supplement or with 1 μM insulin for 30 min. The radioactive uptake was started by addition of KRH [3H]-2-desoxy-D)-glucose ([3H]-2-DG) at an activity of 1 μCi/ml and 50 μM 2-desoxy-D-glucose. Cells were incubated for 30 min and then washed with PBS. The cells were scraped off, after addition of 200 μL IGEPAL and 150 μM phloretin. The radioactivity was measured using liquid scintillation counting with an external standard.

For the measurement of glycerol release, cells were washed with PBS and incubated for 3 hours in phenol red free DMEM containing 2% FFA (free fatty acid)-free BSA (Roth, Karlsruhe, Germany). The medium was changed and the cells were incubated for 1 hour without supplement for basal lipolysis or addition of 10 μM Isoproterenol (Sigma-Aldrich, Steinheim, Germany) and 0.5 mM IBMX for stimulated lipolysis. The supernatant was collected for spectrophotometric glycerol measurement in a Sirius tube luminometer (Berthold Technologies, Bad Wildbad, Germany), using the glycerokinase (Sigma-Aldrich, Steinheim, Germany) and the ATP Kit SL (BioThema, Handen, Sweden). Remaining cells were collected for protein quantification and Western Blot analysis in RIPA buffer containing 50 mM TrisHCl (pH 8), 150 mM NaCl, 0.2% SDS, 1% NP-40, 0.5% deoxycholate, 1 mM PMSF, phosphatase and protease inhibitors. Western Blot analysis was performed using a mouse anti-human GAPDH IgG (Ambion-Thermo Fisher Scientific, Waltham, USA) and the Lipolysis Activation Antibody Sampler Kit #8334 (Cell Signaling, Danvers, USA) according to the manufacturer's protocol. Secondary IRDye IgG (LI-COR, Bad Homburg, Germany) were used to generate the fluorescence, detected by the Odyssey scanner (LI-COR, Bad Homburg, Germany).

Relative Gene Expression qRT-PCR

Primer pairs were designed using published nucleotide sequences from the human genome GenBank NCBI/UCSC and ensembl, “primer3input” (Untergasser et al. 2012) was used for primer design, “net primer” (Premier Biosoft, San Francisco, USA) for optimization and “primer blast” NCBI GenBank (Ye et al. 2012) to verify specificity against the gene of interest. Primers against the human target genes LEPTIN (forward TGGGAAGGAAAATGCATTGGG (SEQ ID NO: 69); reverse ATAAGGTCAGGATGGGGTGG (SEQ ID NO: 70)) and GLUT4 (forward CTGTGCCATCCTGATGACTG (SEQ ID NO: 71); reverse CCAGGGCCAATCTCAAAA (SEQ ID NO: 72)) and the reference genes IIPRT (forward TGAAAAGGACCCCACGAAG (SEQ ID NO: 73), reverse AAGCAGATGGCCACAGAACTAG (SEQ ID NO: 74)), PPIA (forward TGGTTCCCAGTTTTTCATC (SEQ ID NO: 75); reverse CGAGTTGTCCACAGTCAGC (SEQ ID NO: 76) and IPO8 (forward CGGATTATAGTCTCTGACCATGTG (SEQ ID NO: 77); reverse TGTGTCACCATGTTCTTCAGG (SEQ ID NO: 78)) were synthesised by Eurofins (Ebersberg, Germany).

Total RNA was extracted using the RNeasy Mini Kit (Qiagen, Hilden, Germany) and 0.5 ug was reverse transcribed using the High-Capacity cDNA Reverse Transcription Kit (Thermo Fisher Scientific, Waltham, USA). qRT-PCR was performed using 96 well plates (black frame, white wells) with Heat Sealing Films, fixed by the 4s2 Automated Heat Sealer (all from 4titude, Surrey, UK). The Maxima SYBR Green Mix (Thermo Fisher Scientific, Waltham, USA) was used for amplification in a qRT-PCR Mastercycler® ep realplex (Eppendorf, Hamburg, Germany), with a denaturation step of 95° C. for 10 min and 40 cycles of 95° C. for 15 sec and 60° C. for 40 sec, followed by a melting curve. Relative gene expression was calculated by the delta delta Ct method (Pfaffl 2001) with a reference gene index of HPRT, PPIA and IPO8.

### Mice

All mice (C57BL/6J) originally were obtained from Charles River Laboratories, Inc. (Wilmington, Massachusetts, USA). To genetically engineer a Cobll1 whole-body knockout (Cobll1−/−) model Applicants used Crispr/Cas9 genome editing system. Male mice were weaned at 4 weeks of age, and body weight was measured every week from 4 to 14 weeks of age. Mice were housed on a 12-hour light/dark cycle with ad libitum access to food (Normal diet: 14% fat, 64.8% carbohydrate, and 21.2% protein, Harlan Teklad). In order to analyze the body fat mass (%), body length (cm), and bone mineral density (BMD, g/cm²) Applicants used the Dual-Energy X-ray Absorptiometry (DEXA) scan. Prior to scanning, animals were anesthetized with ketamine. All procedures were conducted with approval of the Institutional Animal Care and Use Committee (IACUC) of University of Chicago.

### CRISPR/Cas9-Mediated Generation of a Cobll1 Knockout Mouse Model

To confirm directly that ablation of Cobll1 affects T2D-related phenotypes in vivo Applicants applied the CRISPR/Cas9 system to genetically engineered a Cobll1 whole-body knockout (Cobll1−/−) model. Using specific guide RNAs (sgRNAs), Applicants targeted the Cobll1 gene in the C57BL/6 genetic background. Mice homozygous for a Cobll1-null allele are viable with no evidence of embryonic lethality (data not shown). Applicants used guides with the following sequences: gRNA (exon 2) 5′-TTGCTCACTAGTGGGGTCGCAGG 3′ (SEQ ID NO: 79) and gRNA (exon6) 5′-CTTCCTCCGGCCGAGACGAAGGG-3′ (SEQ ID NO: 80).

### Mice Genotyping

The genotypes of Cobll1 mutant mice were determined by PCR amplification of genomic DNA extracted from tails. PCR was performed for 30 cycles at 95° C. for 30 sec, 60° C. for 15 sec, and 72° C. for 30 sec, with a final extension at 72° C. for 5 min. PCR amplification was performed using the primer sets: Forward 5′-AAAAGTTTCCTGATGTGAAAGTCA-3′ (SEQ ID NO: 81) and Reverse 5′ AAAAACAGATGCTCCCCAGA-3′ (SEQ ID NO: 82). The PCR products were size-separated by electrophoresis on a 4% agarose gel for 1 h.

### Mice In Vivo Glucose Tolerance Test

At 16 weeks old, the animals were tested for glucose sensitivity by Intraperitoneal glucose tolerance test (IPGTT). Prior to IPGTT, mice were fasted for 4h and an initial blood glucose reading was taken. This fast was followed by intraperitoneal injection of 2 mg/kg dextrose (Millipore Sigma), and subsequent blood glucose checks using an AccuChek Aviva glucometer (Roche). Blood glucose readings were taken at 15, 30, 60, and 120 min after dextrose injection. After IPGTT, mice resumed a high fat diet. An unpaired two-sided Student's t-test was used to test for significance.

Mouse Real Time qPCR

After establishment of stable Cobll1 knockout mice, the ablation of the Cobll1 expression was confirmed by quantitative RT-PCR in relevant tissues which showed significant decrease in the mRNA fold change of Cobll1 knockout mice compared to WT and heterozygous litter mates. Total RNA was isolated from the inguinal white fat pad (iWAT), kidney and liver using the RNA extraction reagent RNeasy Mini Kit (Qiagen). cDNA synthesis was performed using SuperScript III First-Strand Synthesis System (Thermo Fisher Scientific). Real time qPCR reactions were performed by using SsoAdvanced Universal SYBR Green Supermix. Real time qPCR amplification was performed using the primer sets: qPcrF 5′-CGTCACAGAGCAACAAGACA-3′ (SEQ ID NO: 83) and qPcrR 5′-ACTGAGCACAGAGGAACACG-3′ (SEQ ID NO: 84).

### Mouse RNA-Sequencing

Total RNA was isolated from the inguinal white fat pad (iWAT) using the RNA extraction reagent RNeasy Mini Kit (Qiagen) from adult Cobll1 null mice and WT litter mate. The RNA-sequencing libraries were generated using the NEBNext Ultra™ II RNA Library Prep (New England Biolabs) and were sequenced on Illumina NovaSEQ platform (Illumina).

### Isolation, Culture and Differentiation of Mouse Pre-Adipocytes

Primary adipocytes were isolated from dissected perigonadal white fat pad (pWAT) of 6-week-old mice and digested in 1 g/mL type I collagenase solution (containing 3.5% BSA, v/v) in a 37° C.′ water bath with shaking at 120 rpm for 45 min. The suspension was centrifuged at 250×g for 5 min, and then the cell pellet was resuspended in culture media (DMEM High Glucose, 20% FBS, 100 units/ml penicillin and 0.1 mg/ml streptomycin), was filtered through a 45-μm strainer, and was seeded in 25-cm² flasks. Confluent pre-adipocytes were induced for two days with an adipogenic media (DMEM High Glucose, 10% FBS, Penicillin/Streptomycin (10000U/ml, 10000 ug/ml), 850 nM insulin, 1 nM T3, 500 μM IBMX, 1 μM Dexamethasone, 125 M Indometacin and 1 μM Rosiglitazone), and then switch to differentiation medium (adipogenic media without IBMX, Dexamethasone and Indometacin). Cells were harvested on the 8th day of differentiation and used for further analysis.

### Oil Red O and Glycerol-3-Phosphate Dehydrogenase (GPDH) Assay of Mouse Pre-Adipocytes

Oil Red O staining was used to assess the presence of lipids in mature adipocytes. For Oil Red O staining, cells were washed with phosphate-buffered saline (PBS) and fixed with 4% paraformaldehyde. The fixed cells were then covered with 3 mg/ml Oil Red O dissolved in 60% isopropanol (v/v) for 20 min and then the dye was washed away with H₂O. For determination of GPDH activity Applicants used a commercially available kit from TAKARA Bio Inc. (Shiga, Japan), by monitoring the dihydroxyacetone phosphate-dependent oxidation of NADH at 340 nm. The enzyme activity was calculated by the formula described in the manufacturer's protocol and GPDH activity was expressed as unit/mg of protein.

### Example 3—the Trinity of In Vivo, In Vitro, and Clinical Characteristics

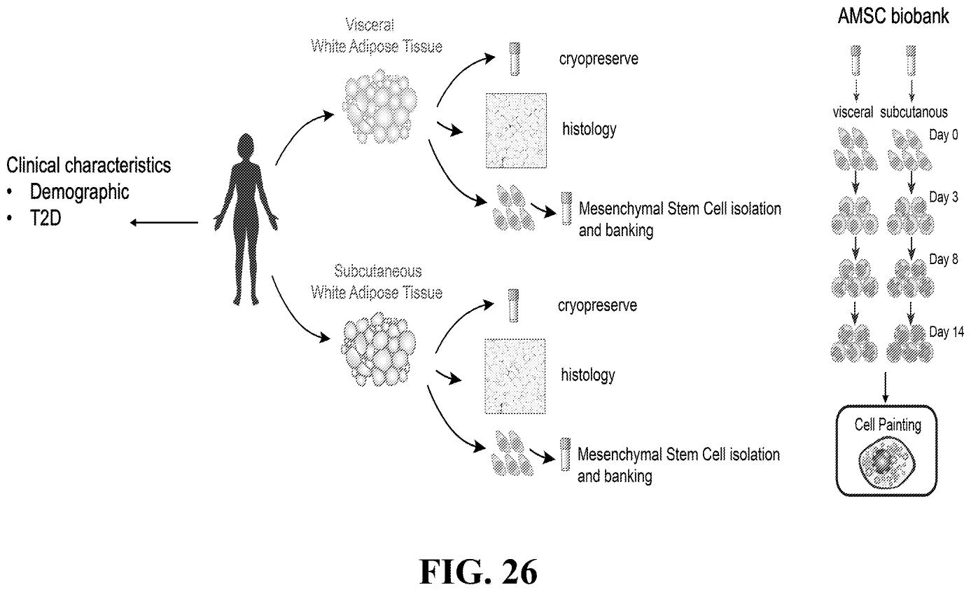

Applicants developed a novel model to link in vitro LipocyteProfiler features to histology cell size estimate features and that these features independently and together can be linked to clinical characteristics. Applicants used a comprehensive and multimodal databank of adipose-derived mesenchymal cells (AMCS) at Melina Claussnitzer Lab (MCL). The databank is a unique resource to investigate associations between in-vivo, cellular, and clinical characteristics of patients. Applicants have used the data to develop a novel analytical pipeline for predicting clinical characteristics in patients. Applicants used datasets for two depots, visceral and subcutaneous adipose cells, containing the cell areas from histology images as reported by Glastonbury C A, Pulit S L, Honecker J, et al. (Machine Learning based histology phenotyping to investigate the epidemiologic and genetic basis of adipocyte morphology and cardiometabolic traits. PLOS Comput Biol. 2020; 16 (8): e1008044. Published 2020 Aug. 14. doi: 10.1371/journal.pcbi.1008044), morphological features identified by LipocyteProfiler (see, example 1), and clinical characteristics of 32 patients (FIG. 26). Applicants showed potential associations between clinical characteristics, in vivo features, and in vitro features. Applicants can identify associations between the three categories of in-vivo (histology-derived features), in-vitro (LipocyteProfiler-derived features), and patient clinical characteristics.

Applicants first confirmed the known associations between histology-derived features and BMI of patients, and showed that the computational pipeline could identify novel associations between histology-derived features and type-2 diabetes (T2D) in visceral samples. Applicants show some of the associations between in-vitro cellular features and clinical characteristics in Example 1, and expanded upon these results by identifying novel associations between the cellular traits and in-vivo histology derived traits. Applicants show that in-vitro features can be used to estimate histology features (mainly in subcutaneous depot) and similarly the in-vivo features can be used to estimate a diverse set of cellular features in both depots and during the examined differentiation time points (days 0, 3, 8, 14).

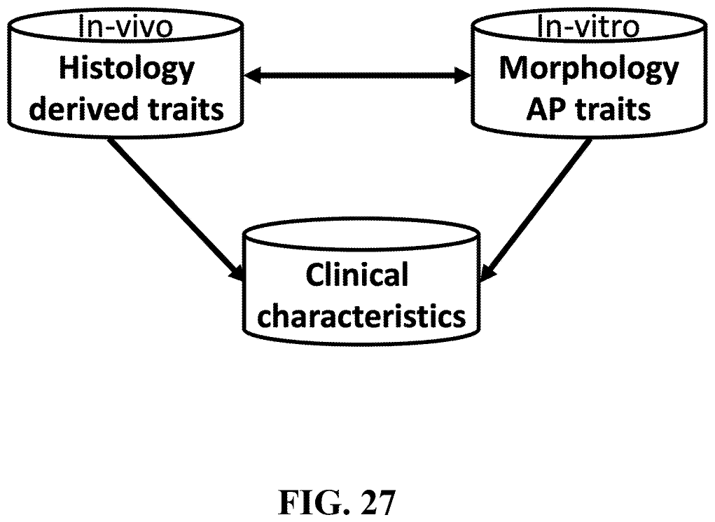

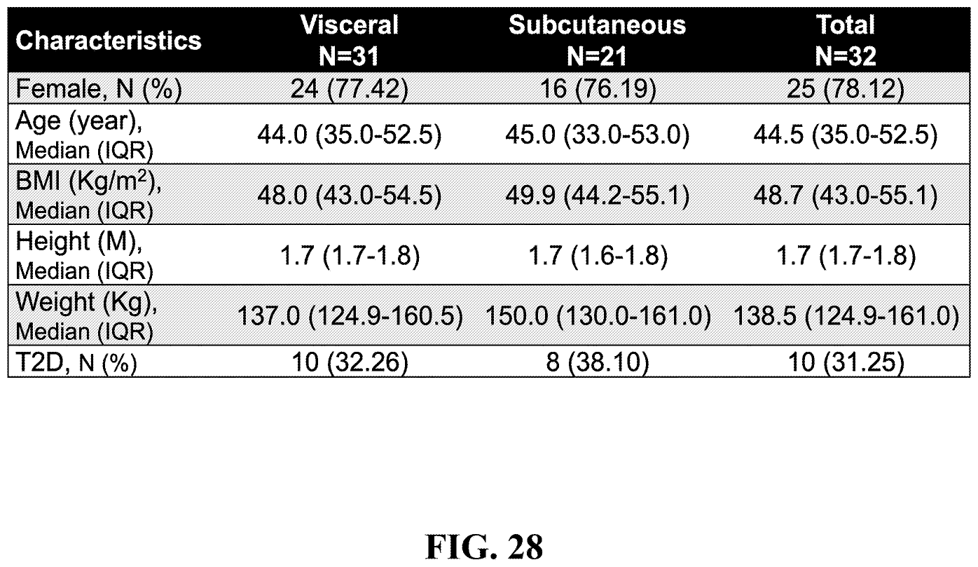

Applicants hypothesized that by using linear models with an expanded set of features, associations between the traits can be identified (FIG. 27). Applicants developed a method that includes: preprocessing of the features, using forward feature selection (AIC stop condition), fitting a generalized linear model, sensitivity analysis on female subjects, and evaluating the models using Pearson correlation with adjusted-p and AUC when applicable. The specificity and sensitivity of the models can be increased by increasing the number of subjects used to develop the models. In one example, Applicants used clinical characteristics that include demographic variables and T2D (FIG. 28).

### Preprocessing

The method for predicting any of in vitro, in vivo and clinical characteristics uses preprocessing pipelines. Applicants used two preprocessing pipelines to prepare the in-vivo histology traits, from the Adipocyte U-Net, and in-vitro cellular traits, from the LipocyteProfiler pipeline outputs.

### In Vivo Traits

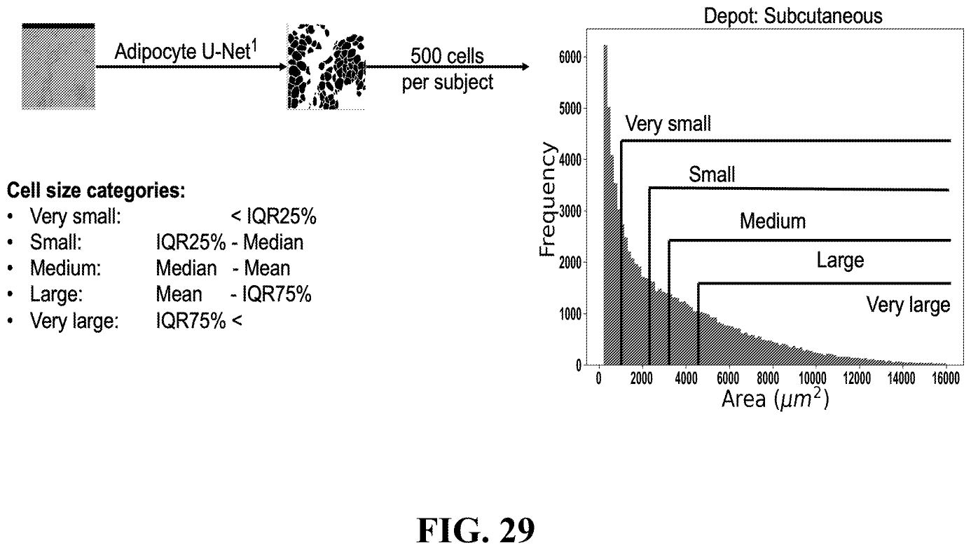

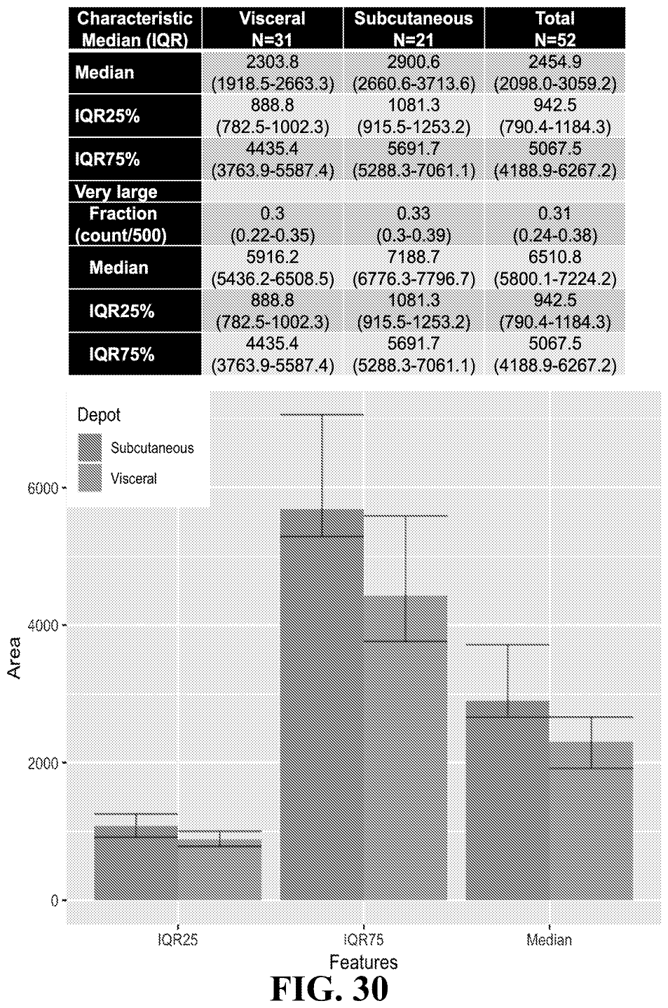

In-vivo histology traits were processed to generate histology features. Applicants previously showed the association between the mean cell sizes and BMI. In order to increase the dimensionality of the features extracted from histology images and to be able to predict further clinical characteristics Applicants defined five cell-size categories and calculate four features per category. Adipocyte U-Net reported 500 cell areas (μm²) per patient. For each depot, Applicants calculated the mean, median, and 25% and 75% quartile points of the 500 cell areas. These values were then used to define five cell-size categories of ‘very small’, ‘small’, ‘medium’, ‘large’, ‘very large’ per depot (‘very small’: cell area <25% point; ‘small’: 25%≤cell area <median; ‘medium’: median≤cell area <mean; ‘large’: mean≤cell area <75% point, and ‘very large’: 75% point≤cell area). For every sample, Applicants grouped the 500 cell areas into the five categories, and for each category Applicants calculated (i) a fraction of the number of cells in the category over 500, (ii) median area, and (iii) the 25 and (iv) 75% interquartile points. Therefore, the histology traits of every sample are captured and represented with 20 features (5 categories×4 variable) (FIG. 29). Examples of features used to represent histology images is shown (FIG. 30).

### In Vitro Traits

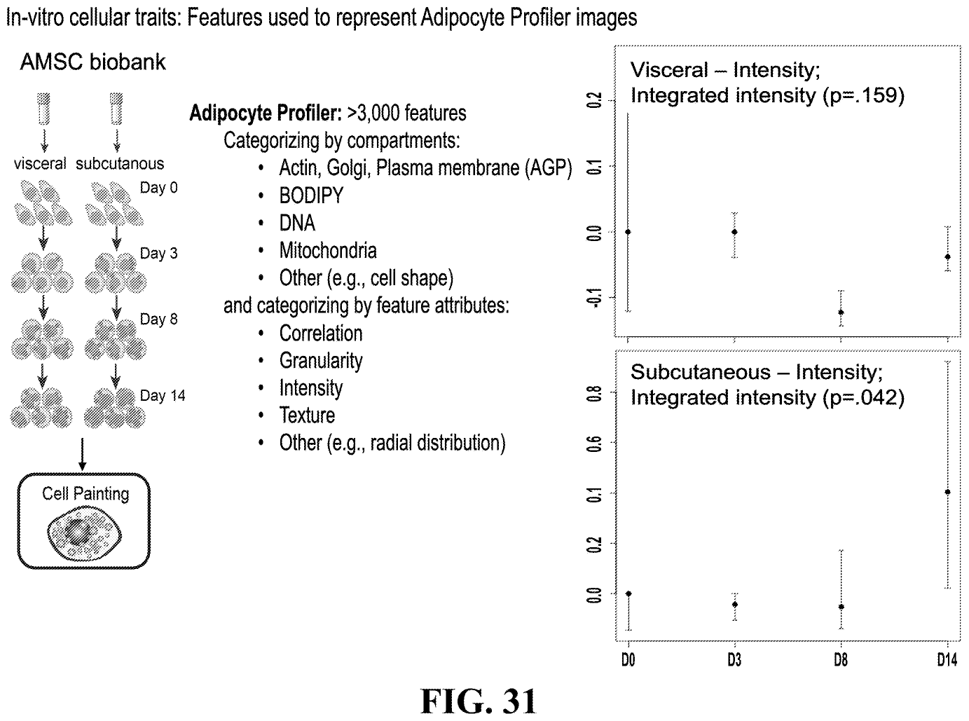

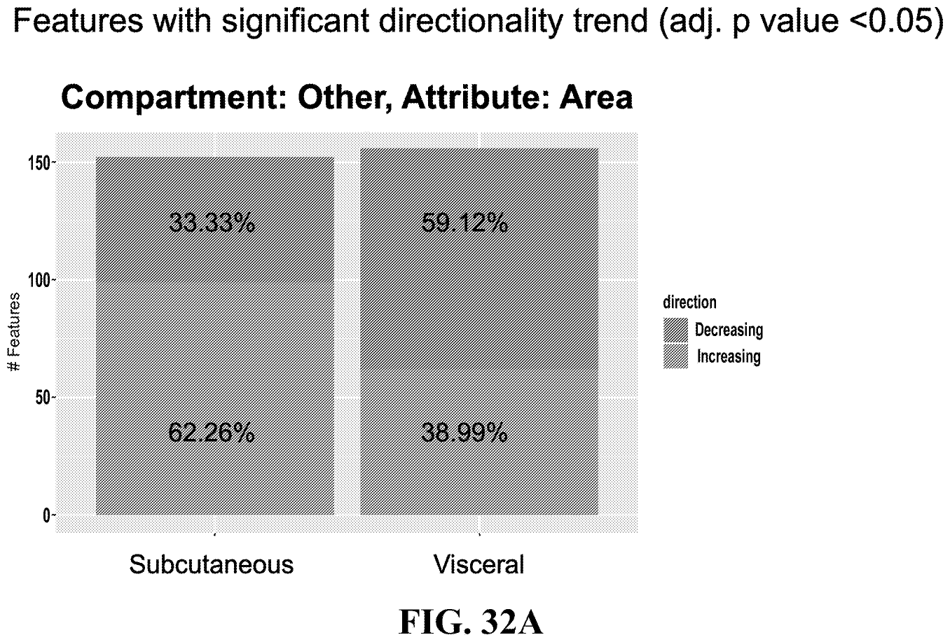

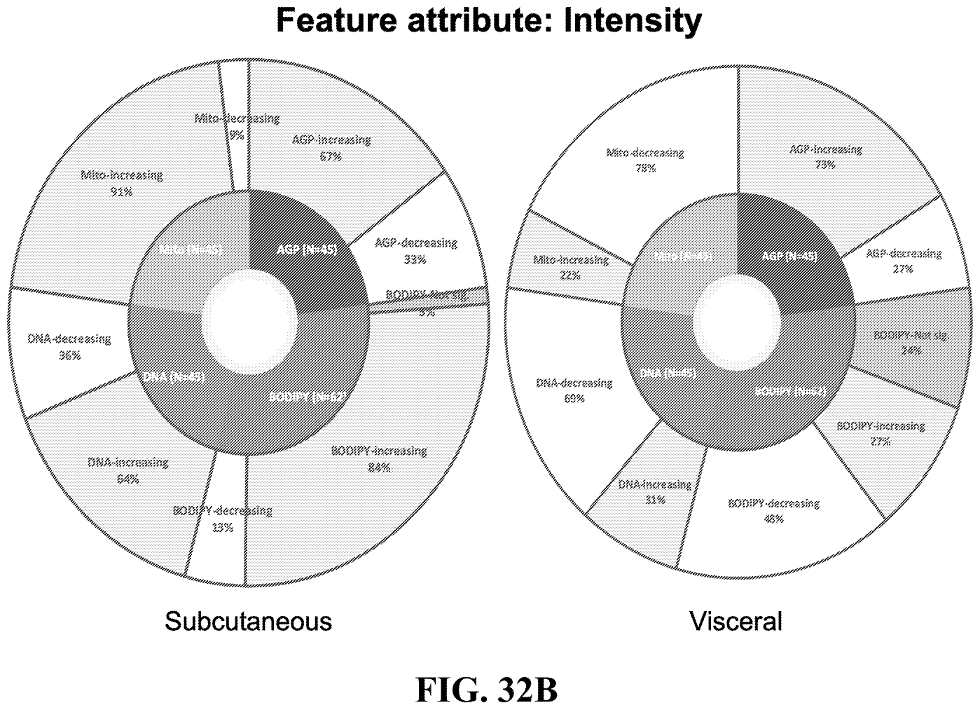

In-vitro cellular traits were processed to generate morphology features. Examples of features used to represent LipocyteProfiler images is shown (FIG. 31). There are correlated features in the set of features generated using LipocyteProfiler, and the large number of features (n=3005) for a relatively small set of samples (N=193) could yield to misleading analysis. Example features with significant directionality trend (adj. p value <0.05) are shown (FIG. 32A-B). To select a subset of representative features, Applicants used the ARACNE (Algorithm for the Reconstruction of Accurate Cellular Networks, BMC Bioinformatics 7, S7, doi: 10.1186/1471-2105-7-S1-S7, 2006) software package. ARACNE was originally developed to address network deconvolution problems in regulatory networks, and showed promising results for identifying transcriptional interactions. ARACNE was used to construct an interaction network between the features measured by LipocyteProfiler. The nodes of this network represent the features, and every edge of the network indicates an interaction between two nodes. ARACNE assigns weights to the edges that can be considered as the importance of the interactions for reconstructing the network. Applicants applied a cutoff of <0.4 on the edges to removed low-weight interactions. The nodes in the graph with at least one edge were used to select a subset of cellular traits.

### Trinity Associations

Applicants examined associations between the three datasets (in-vitro and in-vivo imaging traits and the clinical characteristics of the patients). This contains four sets of analyses: Applicants investigated estimating every variable from the clinical characteristics using (i) in-vitro and (ii) in-vivo imaging traits, (iii) estimating in-vivo imaging traits using the in-vitro variables, and (iv) estimating in-vitro imaging traits using the in-vivo variables.

Applicants used the analysis for estimating clinical characteristics from the in-vivo traits as an example, and this process applies to all four sets of analyses. To estimate a clinical characteristic, a logistic regression model (a generalized linear model with logit link (GLM)) was fit on the entire set of the imaging traits. The linear association with binomial distribution was implemented using the R glm function. The default glm convergence criteria on deviances was used to stop the iterations. The DeLong method was used to calculate confidence intervals for the c-statistics. The Bonferroni adjusted Pearson correlation between the actual and estimated values are also reported. For every clinical variable, Applicants used forward feature selection (R step function) to select the most important imaging traits. The Akaike information criterion (AIC) was used as the stop condition for the feature selection procedure. The R function preProcess was used to normalize (center and scale) the non-dichotic variables.

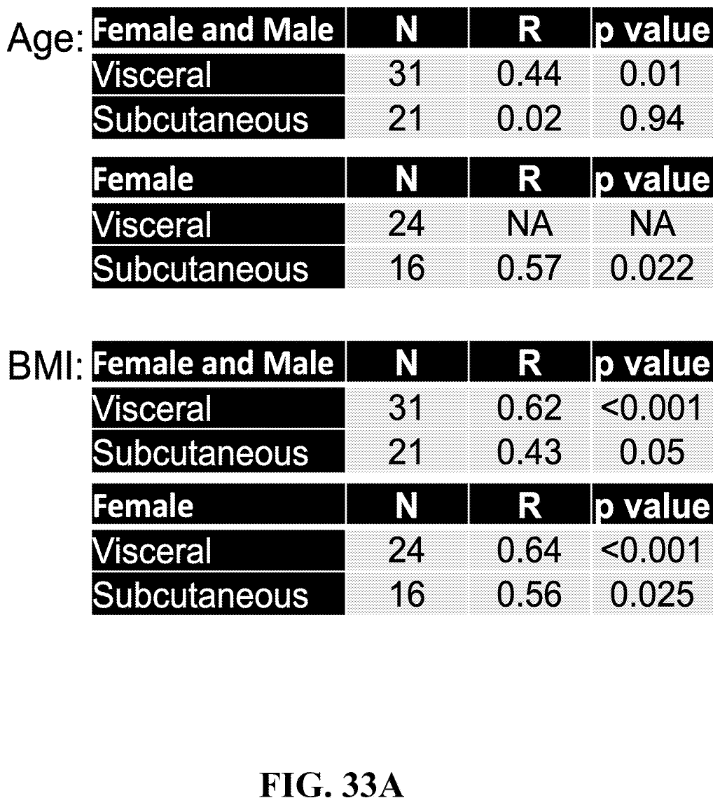

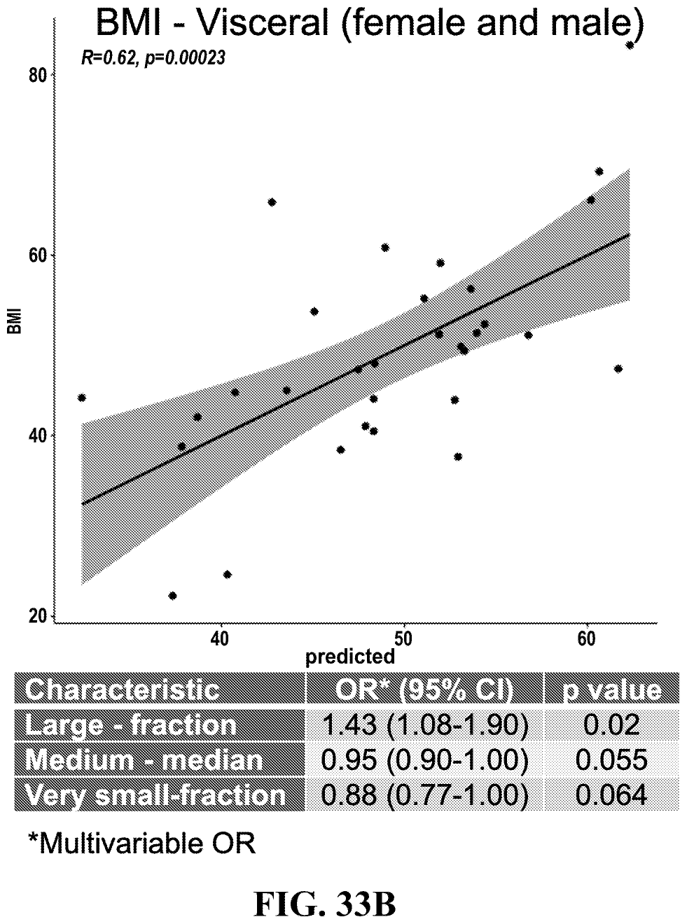

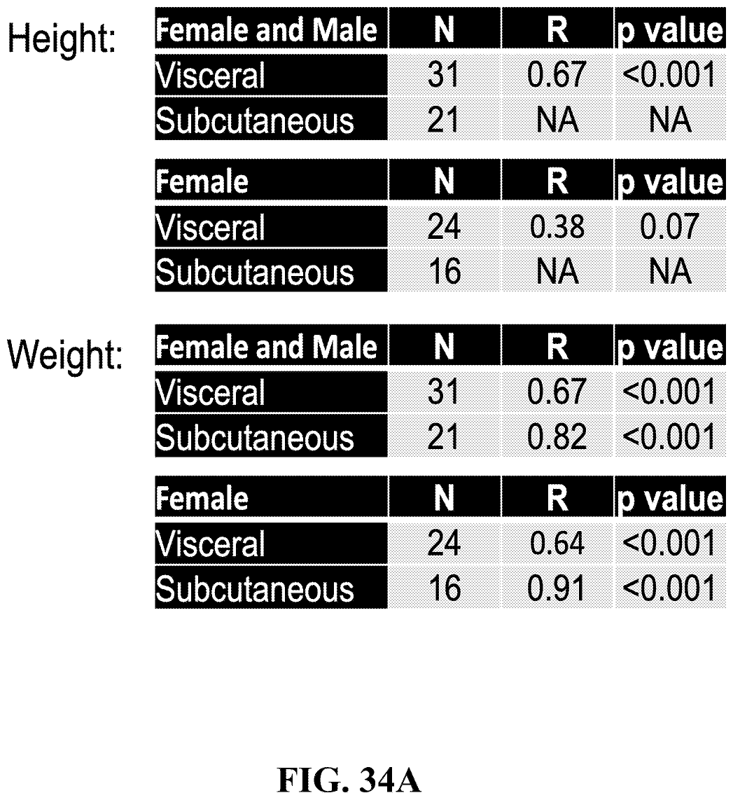

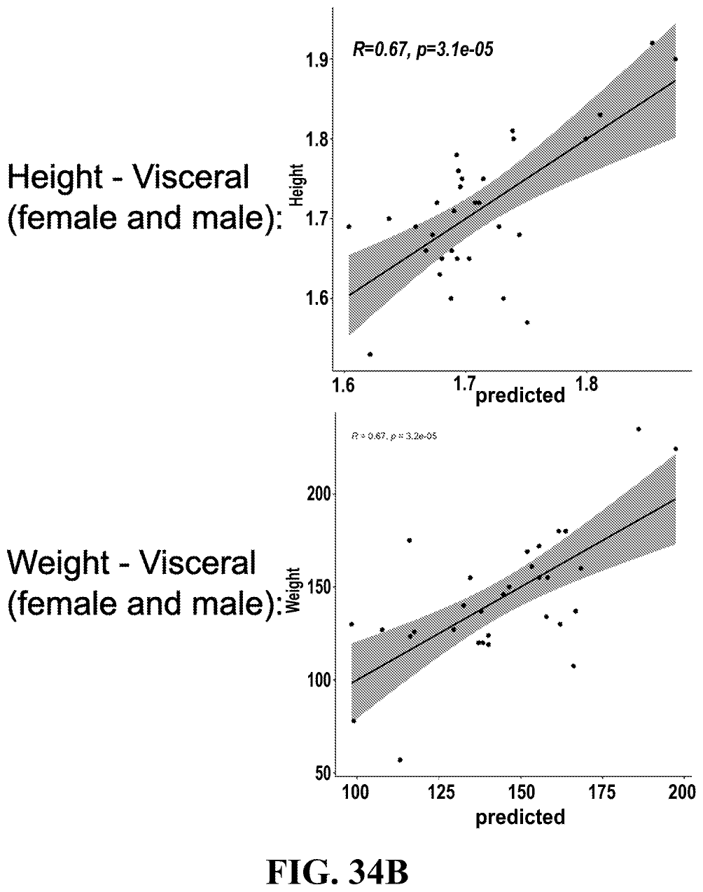

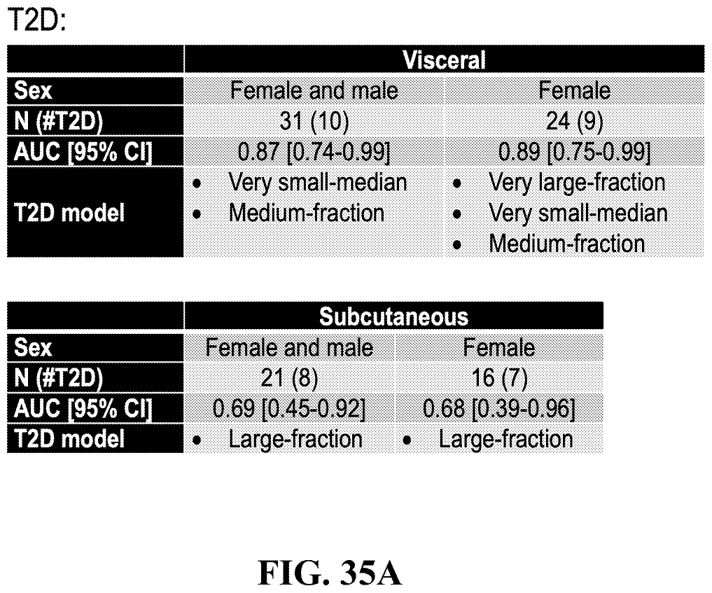

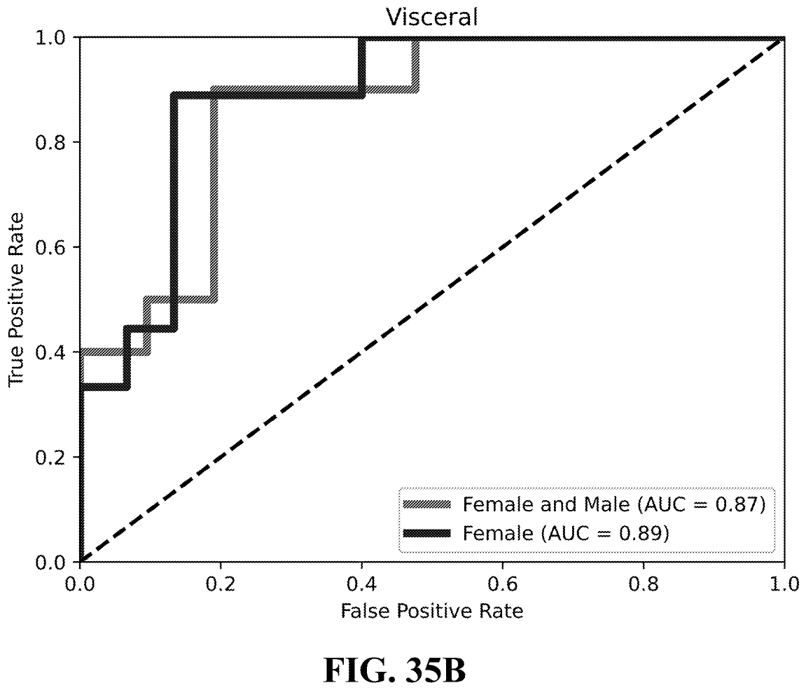

Applicants used histology-derived size estimates to model clinical characteristics. FIG. 33A-B shows prediction of age and BMI using histology-derived size estimates. The prediction is compared to the actual clinical characteristic and R and p values are provided. The large-fraction feature provides for the highest overall risk. FIG. 34A-B shows prediction of height and weight using histology-derived size estimates. FIG. 35A-B shows prediction of T2D) using histology-derived size estimates. AUC is shown to indicate the sensitivity and specificity of the prediction. Traits in visceral AMSCs could predict T2D with an AUC of 0.87. As an example, the model for T2D can use the features of very small-median and medium-fraction.

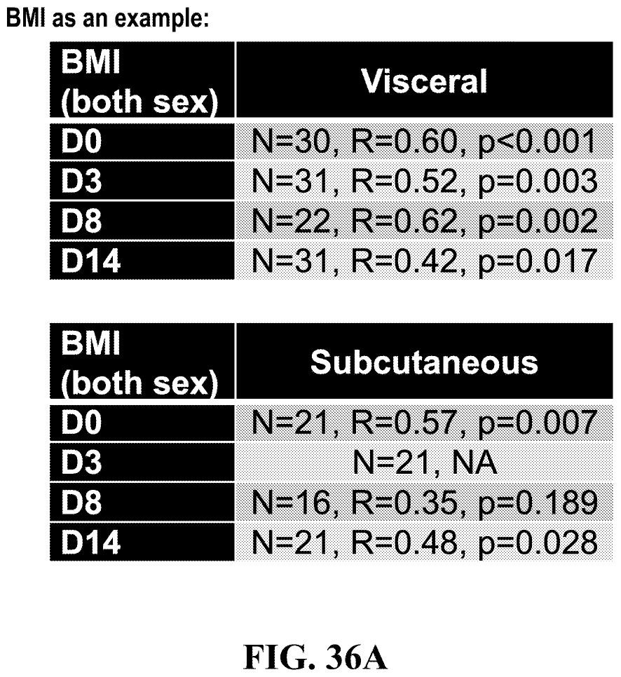

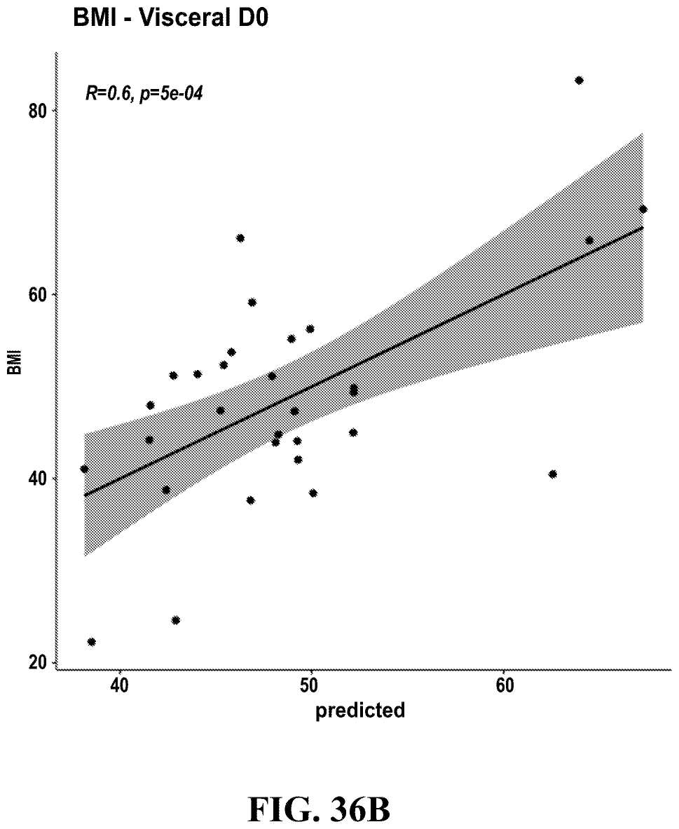

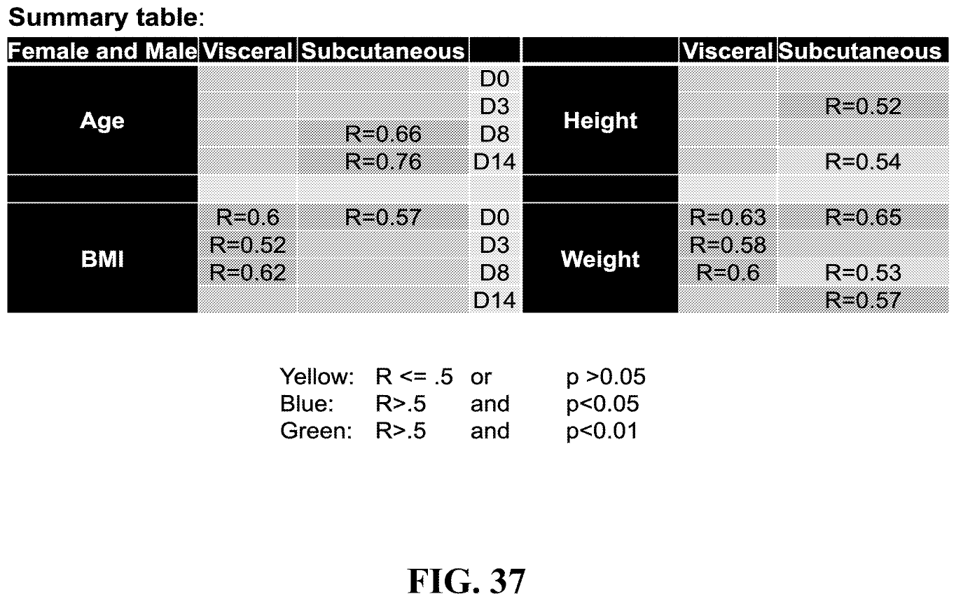

Applicants also used LipocyteProfiler traits to model clinical characteristics. FIG. 36A-B shows prediction of BMI using LipocyteProfiler traits. FIG. 37 shows a summary of predictions made for age, BMI, height and weight using LipocyteProfiler traits. R values greater than 0.5 and p values less than 0.05 are shown for traits in cither visceral or subcutaneous depots and at the differentiation timepoints. In one example, the D14 timepoint in subcutaneous AMSCs has an R value greater than 0.5 and a p value less than 0.05 for height. In another example, the D8 timepoint in subcutaneous AMSCs has an R value greater than 0.5 and a p value less than 0.05 for weight. Every other trait shown has an R value greater than 0.5 and a p value less than 0.01.

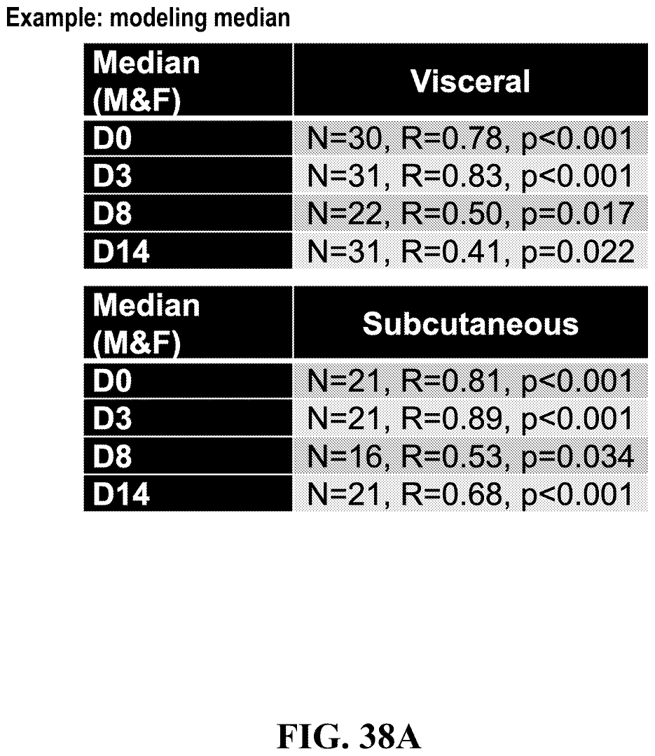

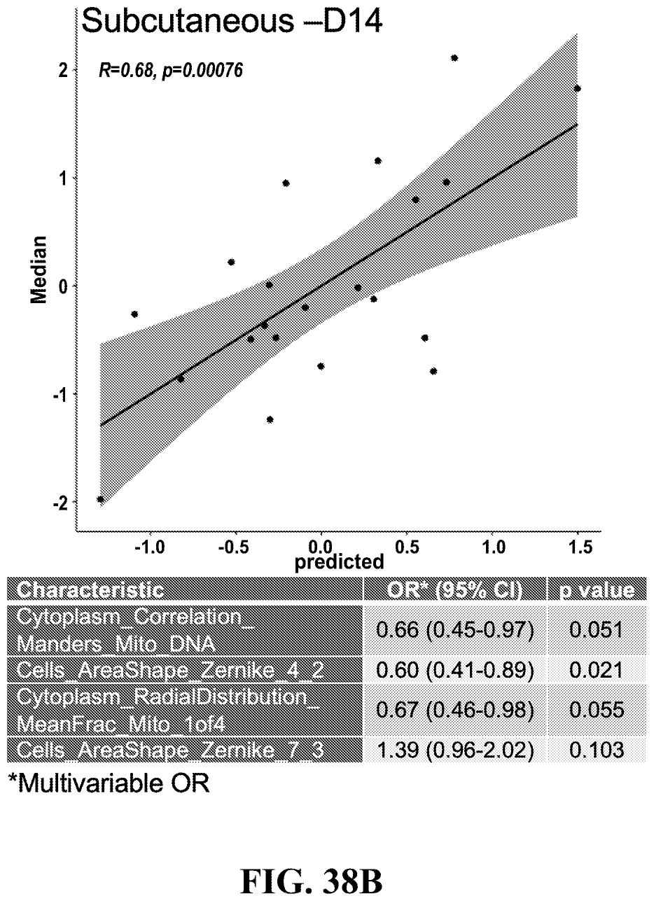

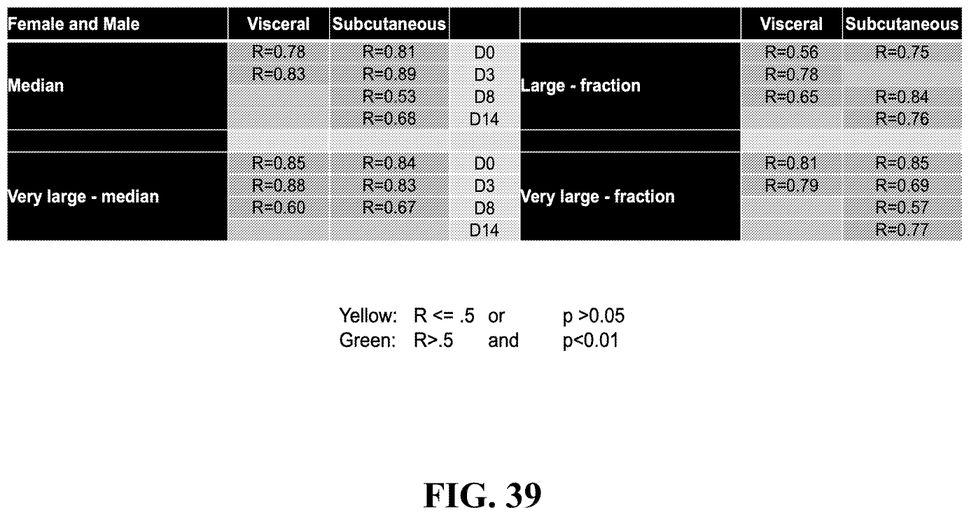

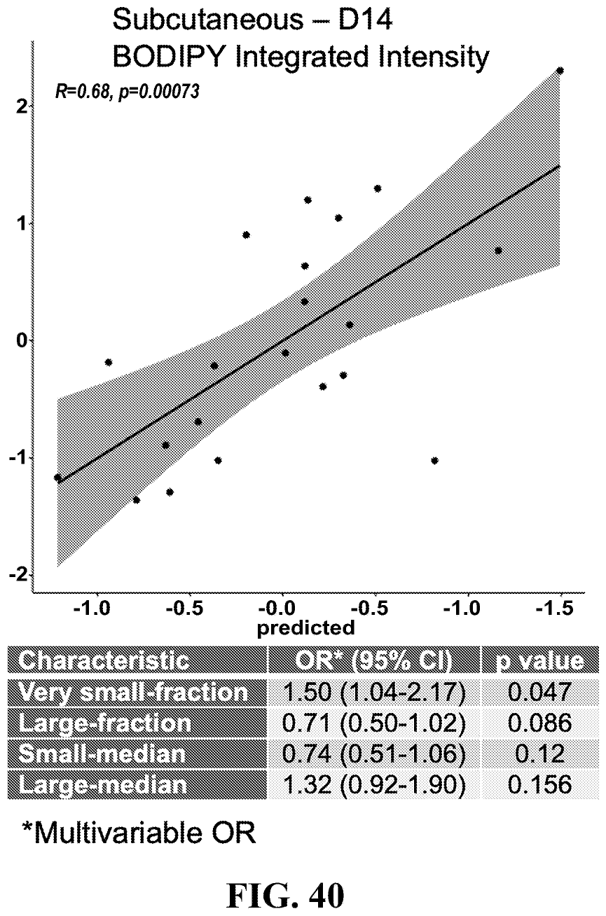

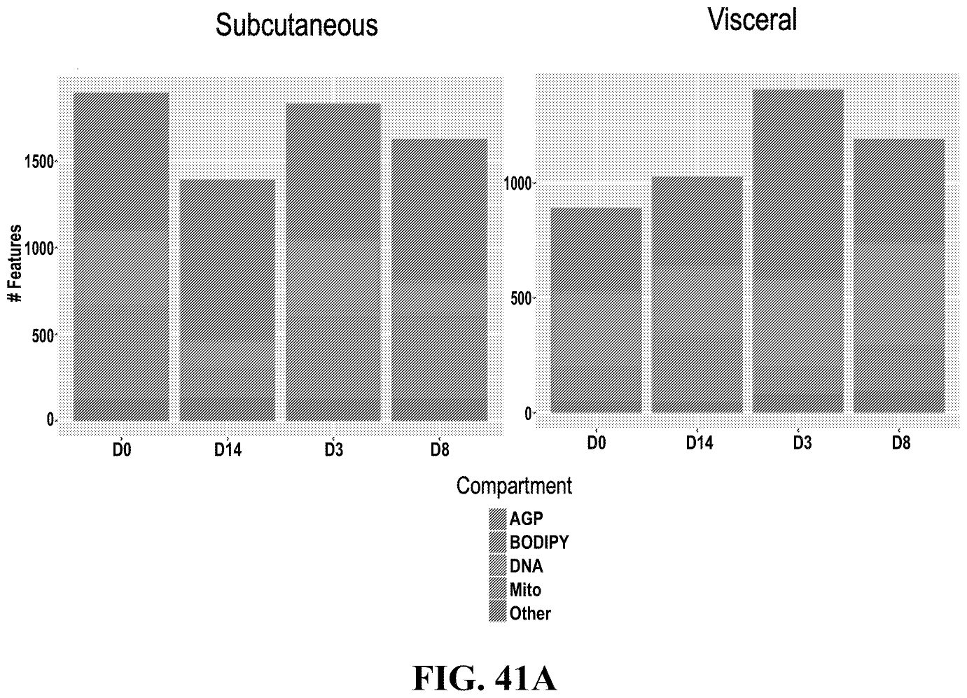

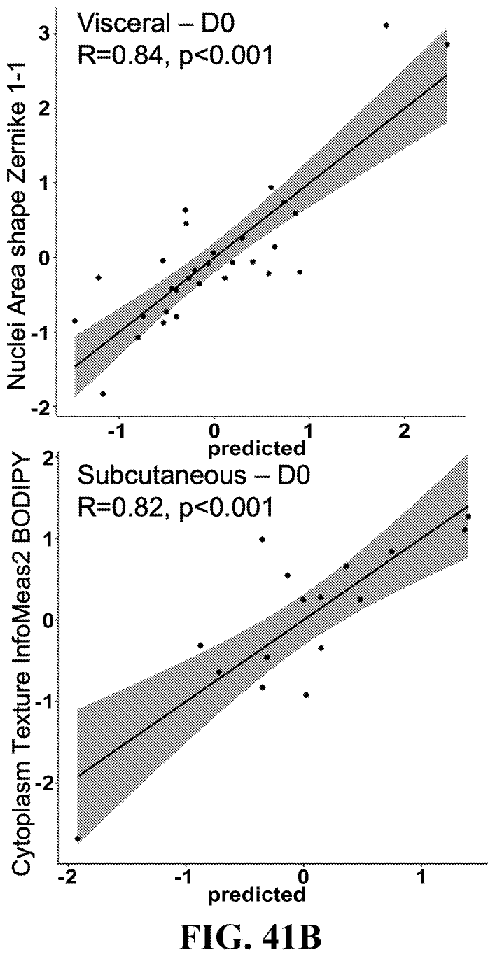

Applicants also used LipocyteProfiler traits to model histology-derived size estimates (FIGS. 38A-B and 39). Applicants also used histology-derived size estimates to model LipocyteProfiler traits (FIGS. 40 and 41A-B). Instead of reporting (R, p) for every ˜3,000 cellular traits: traits are grouped by compartment categories (AGP, BODIPY, etc.) and stratified by differentiation days. The method can report the number of traits that could be modeled (R>0.5, adjusted p<0.05) in every grouped and stratified sub-cohort. The number of traits for each compartment category can be used to predict clinical characteristics. FIG. 41 shows that using the number of modeled morphological traits with an R value greater than 0.5 and an adjusted p value less than 0.05 can be used for the prediction.

### Discussion

Applicants used clinical characteristics, histology, and Adipocyte Profiler derived morphological traits to study associations between the traits. Applicants developed methods of modeling clinical characteristics. In one example, histology adipocyte size traits were used. While most of the clinical characteristics could be modeled using the visceral adipose samples, the models on the subcutaneous samples showed partial success for BMI, weight, and T2D. In another example, Adipocyte Profiler traits were used. Most clinical characteristics could be modeled at some scattered differentiation time points. Applicants observed no trend in the success rate of the models.

Applicants also show modeling histology-derived adipocyte size traits using in-vitro Adipocyte Profiler features. Higher rates of success were observed during early differentiation days using the visceral cohort. Alternatively, using the subcutaneous cohort the traits could be modeled at almost all time points.

Applicants also show modeling cellular adipocyte traits using histology-derived size estimates. A variety of traits from the compartment subgroups (AGP, BODIPY, DNA, Mito, Other) could be modeled at different differentiation time points.

The modeling of clinical characteristics using histology-derived adipocyte traits align with the current knowledge. The results on connecting in-vitro Adipocyte Profiler high-content imaging traits to clinical traits is shown herein for the first time. Histology-derived size estimates can be modeled by in-vitro Adipocyte Profiler traits, which validates the in-vitro adipocyte model system. The results show novel modeling of in-vitro Adipocyte Profiler traits using histology-derived adipocyte size estimates.

### Example 4—Pervasive Pleiotropy Mediates Metabolic Risk at the Rs12454712 Locus (BCL2, KDSR, and VPS4B)

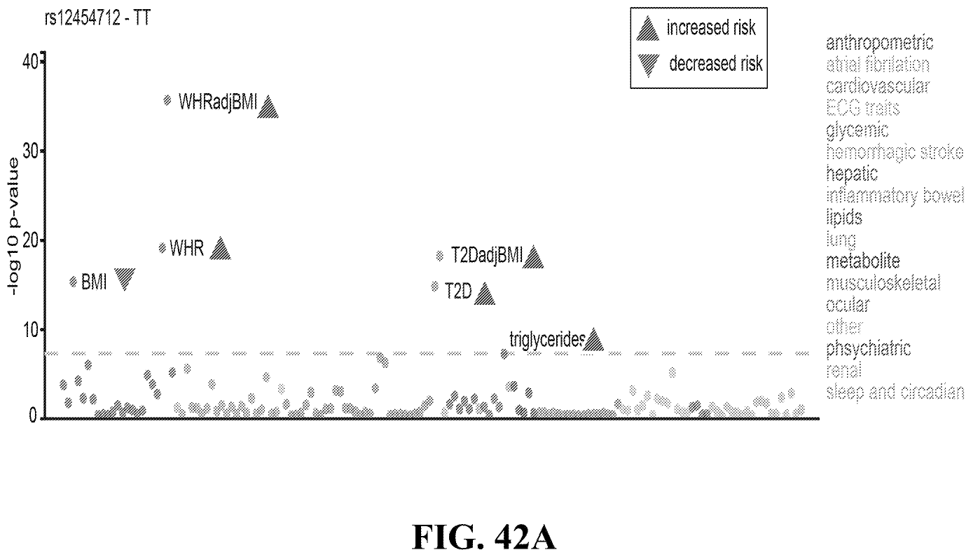

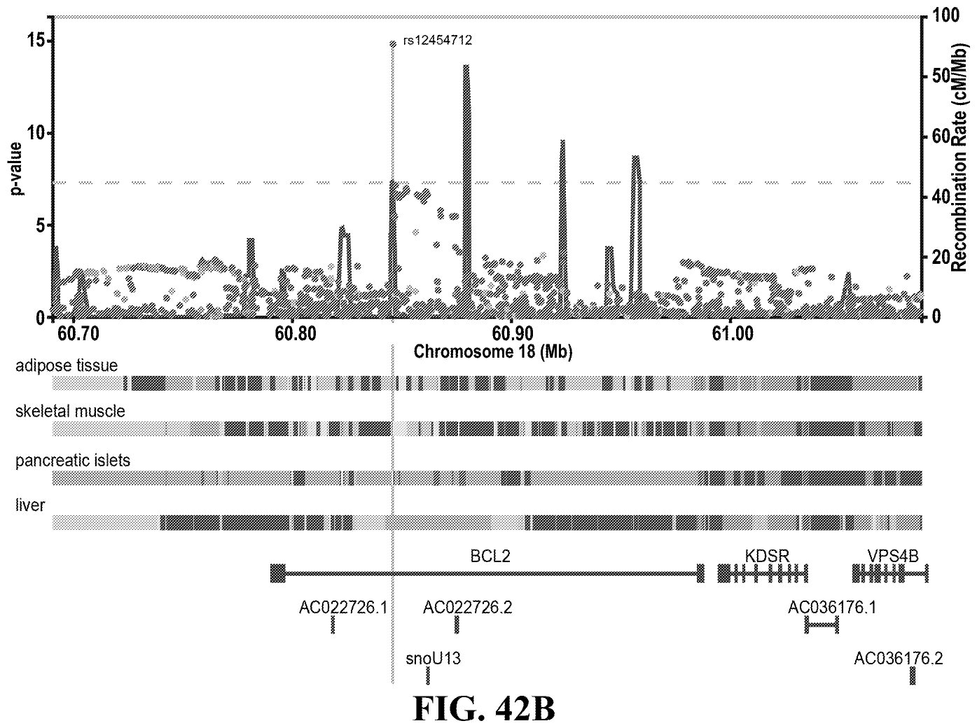

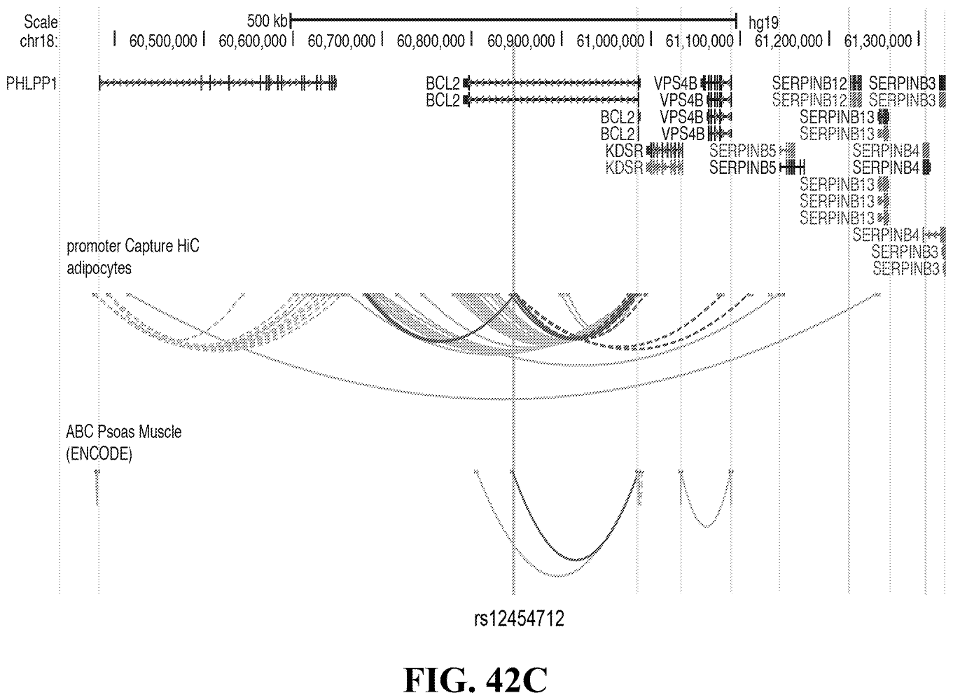

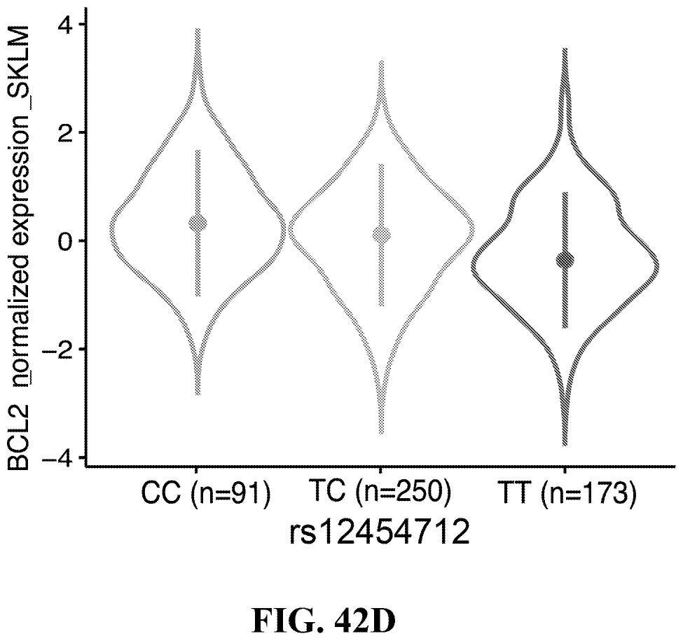

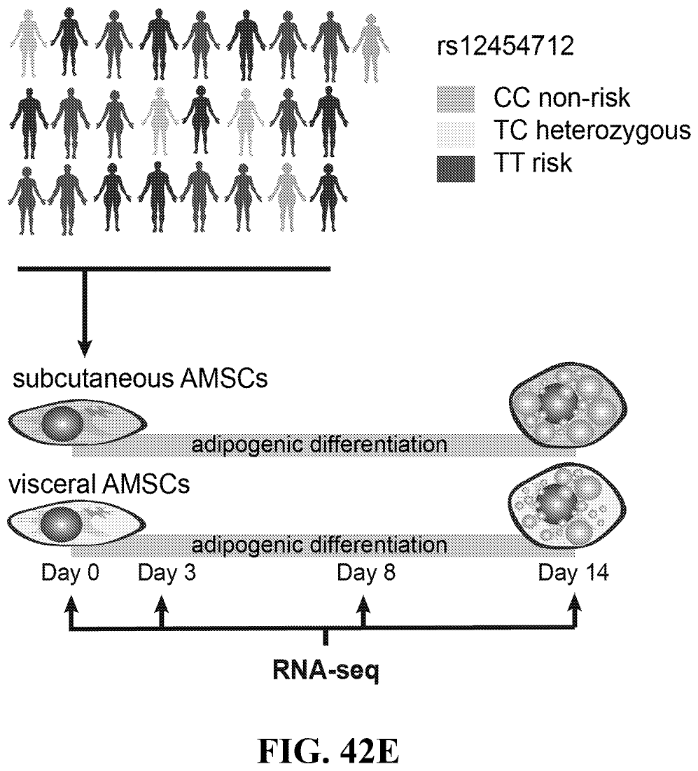

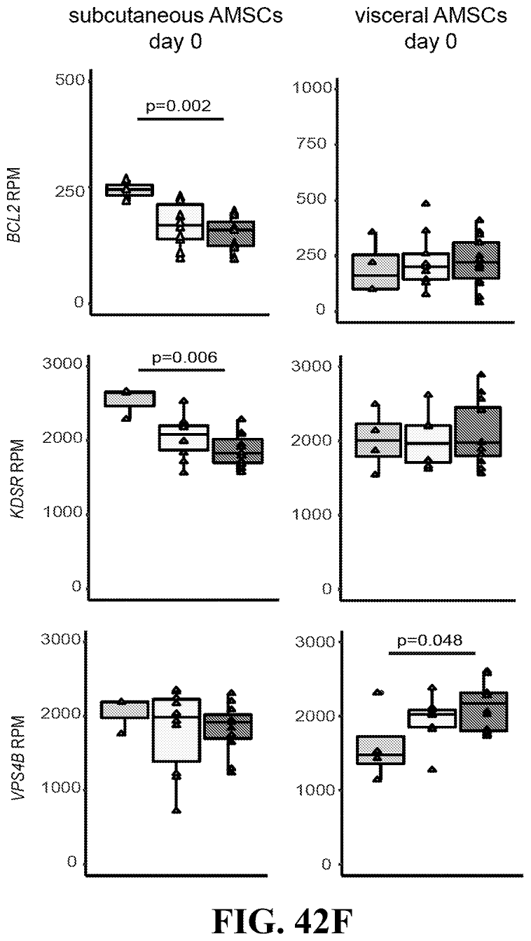

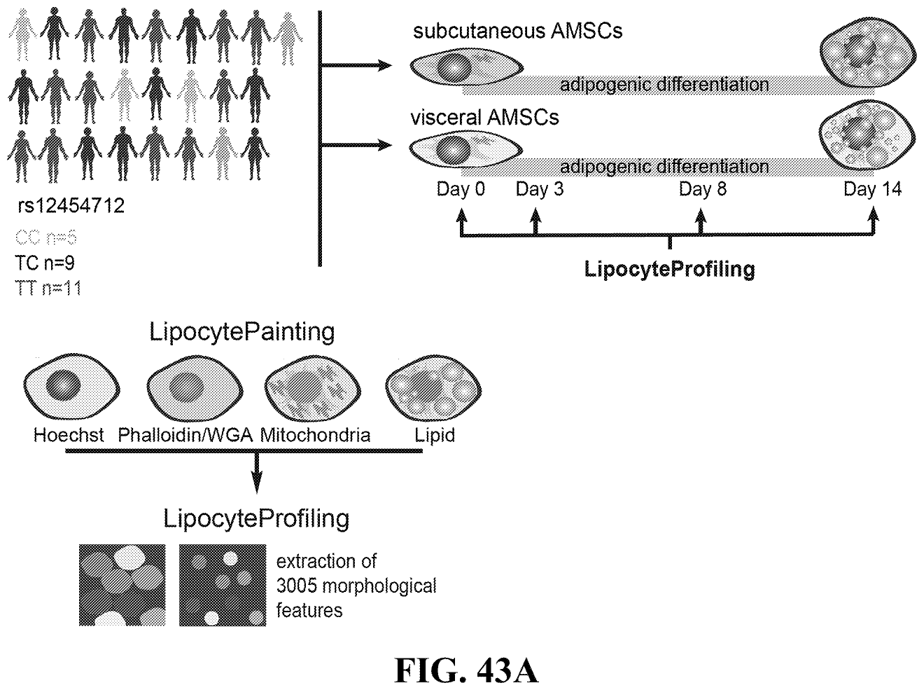

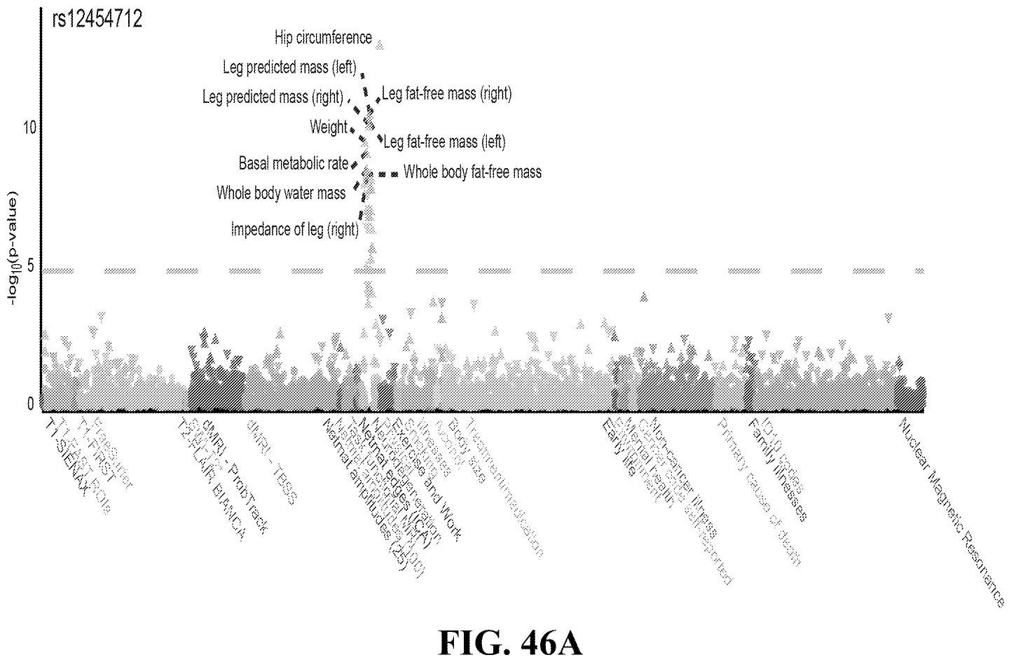

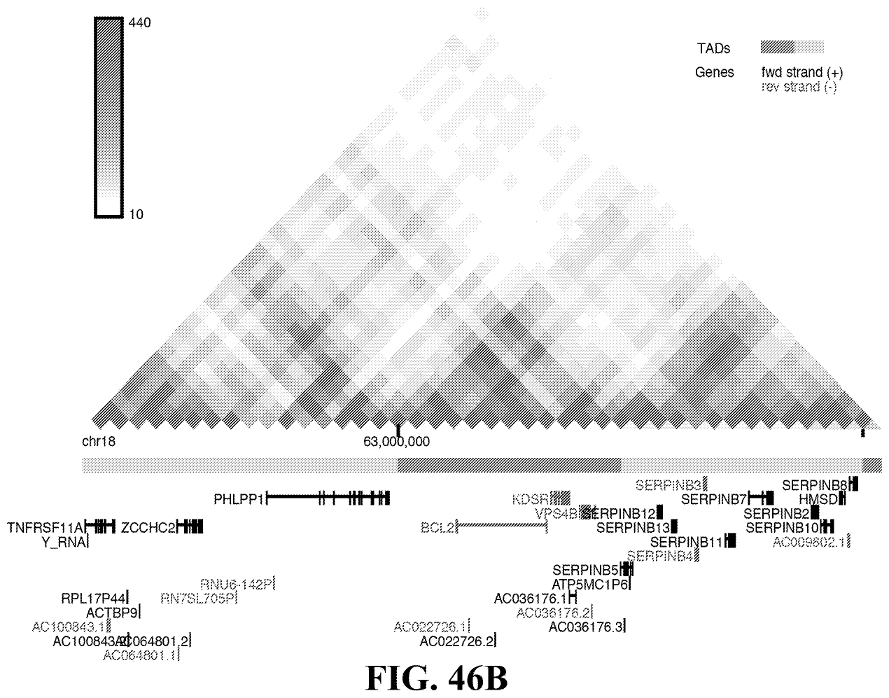

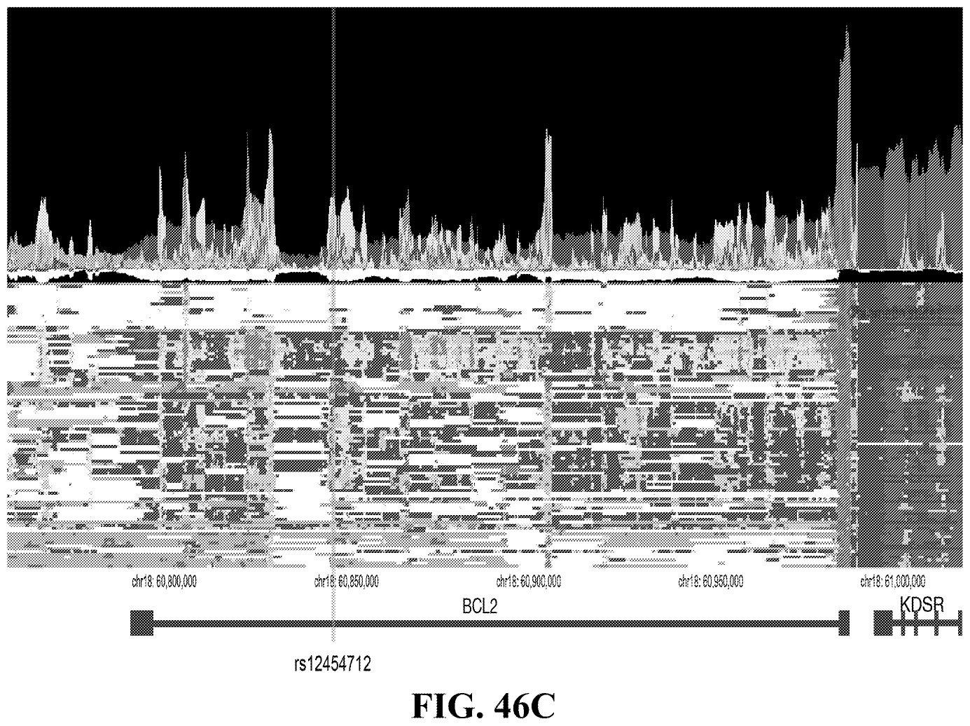

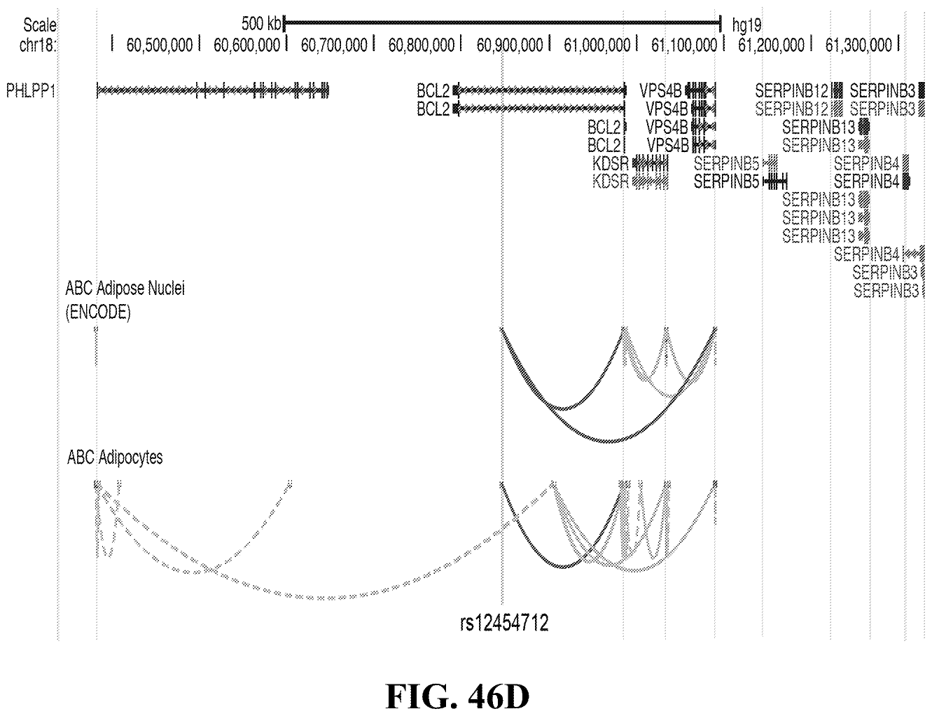

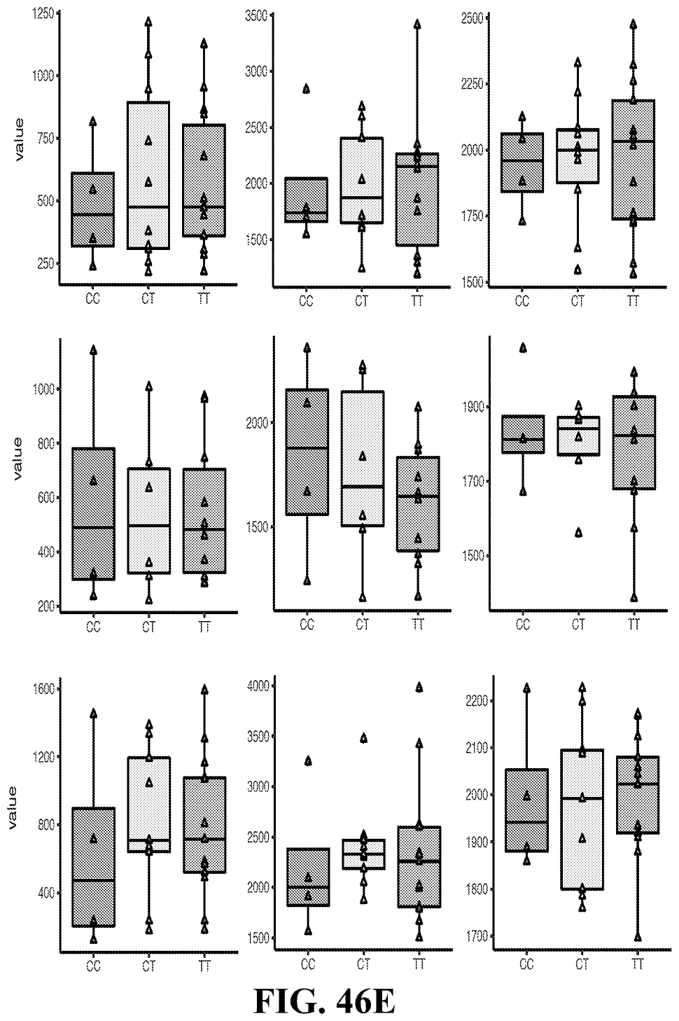

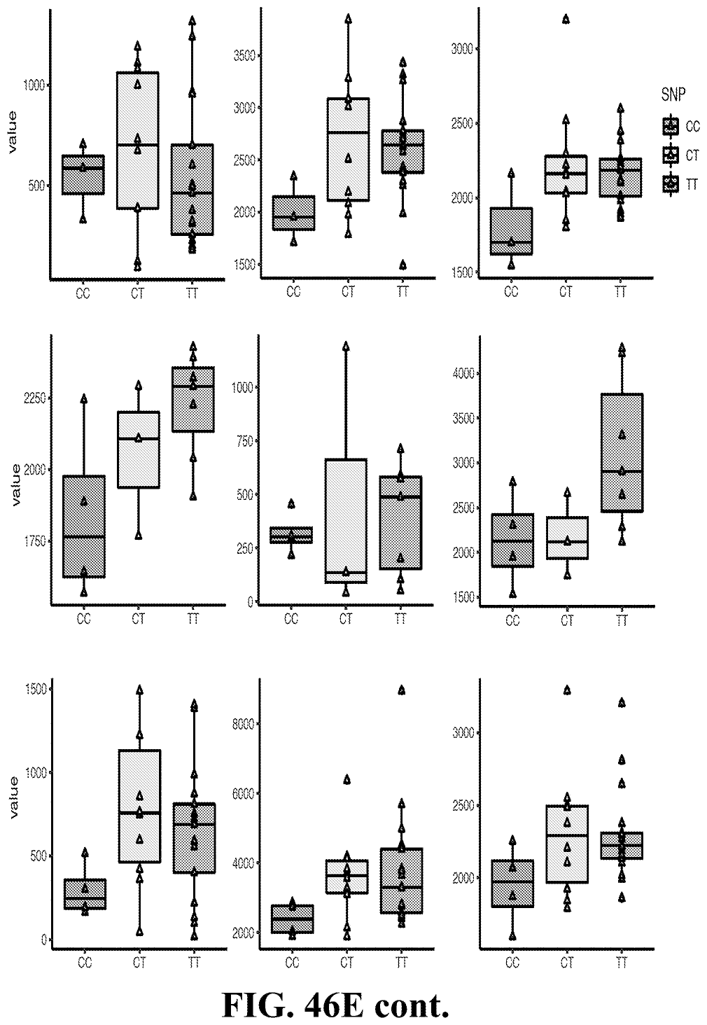

Using PheWAS jointly analyzing many traits and disease states (Taliun et al. 2020), Applicants found rs12454712 to be associated with a number of metabolic traits (FIGS. 42A and 46A), including insulin sensitivity (Walford et al. 2016), BMI-adjusted T2D) (Mahajan et al. 2018), and BMI-adjusted waist-to-hip ratio (WHRadjBMI) (Pulit et al. 2019). Together this indicates a locus where the major T allele associates with a lean metabolically unhealthy phenotype consistent with a clinical presentation of lipodystrophy. The 18q21.33 locus contained no other variants linked to the lead variant, suggesting that rs12454712 is the causal variant mediating disease risk (FIG. 42B). To identify the likely causal tissues of action, Applicants next overlapped rs12454712 with chromatin state maps across 833 reference epigenomes (Boix et al. 2021) and found that this locus maps to an active regulatory element (FIG. 46C). When comparing epigenomic maps in the four most relevant T2D tissues, adipose tissue, skeletal muscle, pancreas and liver (FIG. 42C), Applicants found rs12454712 overlapped active regulatory marks specifically in adipose tissue and skeletal muscle, suggesting that this locus mediates T2D risk through acting in these two tissues. This is consistent with tissue of action (TOA) predictions, which have identified the tissues that most likely mediate T2D risk at this genetic signal to be skeletal muscle and adipose (Torres et al. 2020).

To identify the possible effector transcript(s) mediating risk at the 18q21.33 locus, Applicants next used orthogonal approaches assessing the regulatory architecture surrounding rs12454712. Three-dimensional chromosomal conformation in human mesenchymal stem cells (Dixon et al. 2015) shows that the locus has a concise topologically associated domain (TAD) structure, encompassing three coding genes; BCL2, KDSR, and VPS4B (FIGS. 42C and 46B). In skeletal muscle, activity-by-contact (ABC) (Fulco et al. 2019) target gene predictions and eQTL analyses linked rs12454712 to BCL2 (FIG. 42D), with the TT risk allele being associated with lower BCL2 expression. BCL2 is a crucial regulator of stimulus-induced autophagy in vivo and required for muscle glucose homeostasis (Fernández et al. 2018; He et al. 2012). In adipocytes, promoter Capture Hi-C showed that the variant forms functional connections to the BCL2 promoter (FIG. 42D) and was predicted to regulate BCL2, KDSR and VPS4B using the ABC model (FIGS. 42C and 46D). However, Applicants did not find any eQTLs for rs12454712 in adipose tissue. Given the absence of eQTLs in adipose tissue, Applicants next investigated genotype-driven gene expression differences of potential target genes using RNA-seq from adipose-derived mesenchymal stem cells (AMSCs) of subcutaneous and visceral adipose tissue at four time points of adipogenic differentiation (FIG. 42E). In undifferentiated subcutaneous AMSCs (day 0) BCL2 and KDSR expression were significantly reduced in the TT risk allele compared to the CC allele (FIG. 42F). These allele-specific gene expression changes disappeared with induction of adipogenic differentiation (FIG. 46E). In visceral AMSCs, the TT allele showed increased VPS4B expression at day 0 (FIG. 42F), but had no effect after differentiation was induced (FIG. 46E). Thus, the effect of rs12454712 on BCL2 and KDSR in subcutaneous and VPS4B expression in visceral AMSCs was specific to pre-adipocytes, which is consistent with the absence of eQTLs in mature adipose. Together, these data point towards a regulatory network in which rs12454712 affects at least three target genes in at least three tissues at specific developmental windows. Given that the strongest association for rs12454712 was with body fat distribution, which has been shown to be primarily driven by effects in adipose tissue (Shungin et al. 2015; Pulit et al. 2019), Applicants next set out to dissect the mechanistic underpinnings of rs12454712 in adipocytes from both subcutaneous and visceral adipose depots.

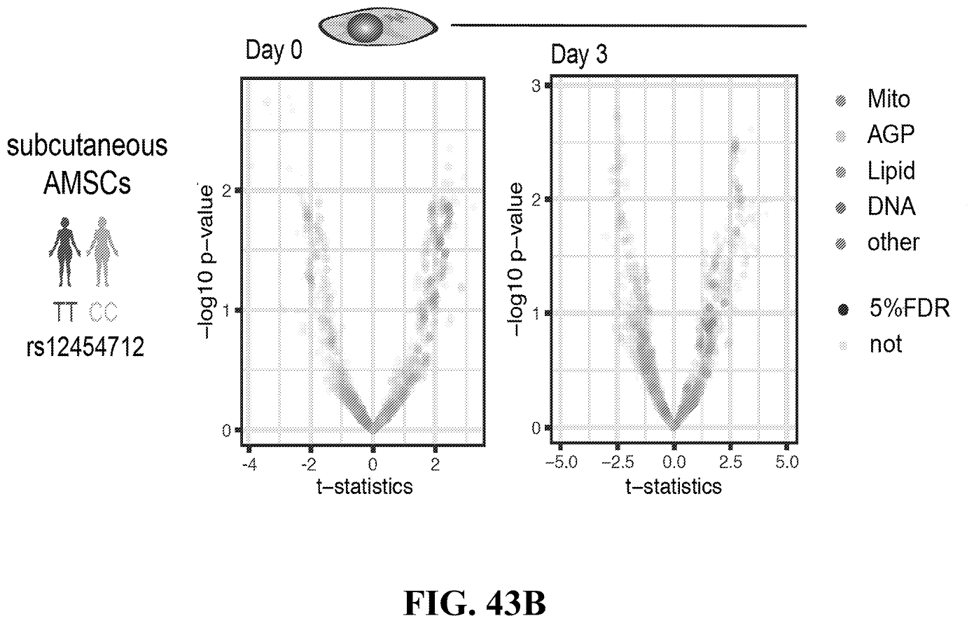

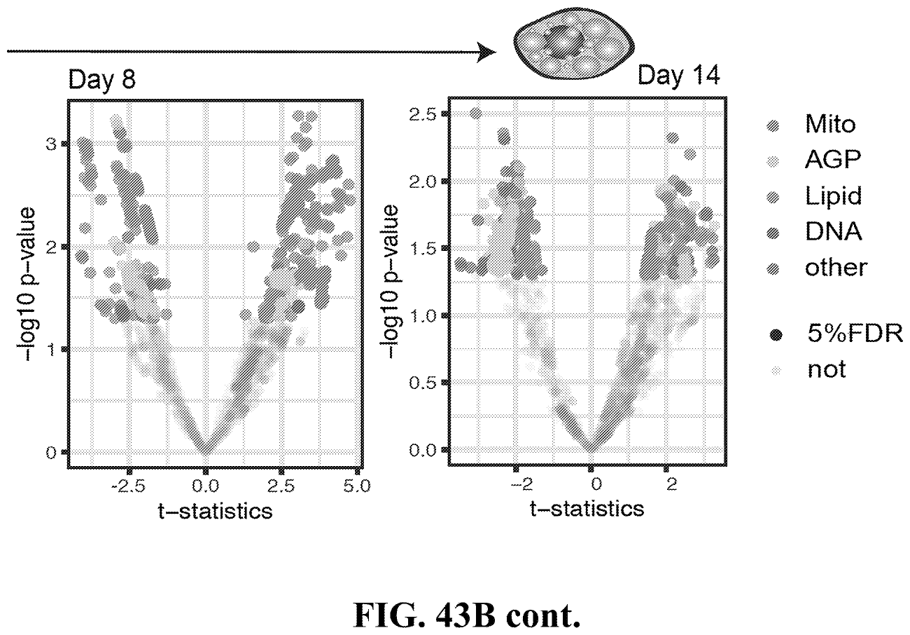

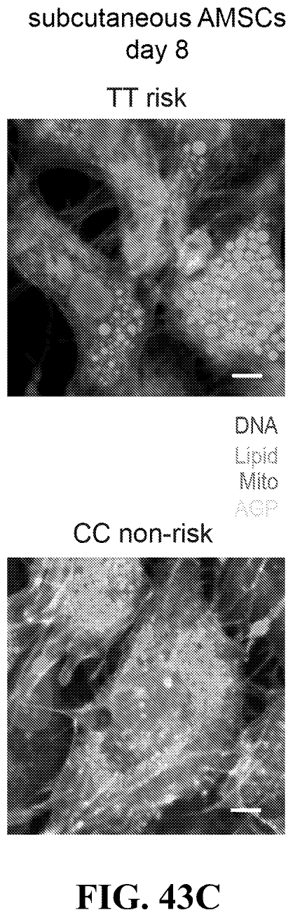

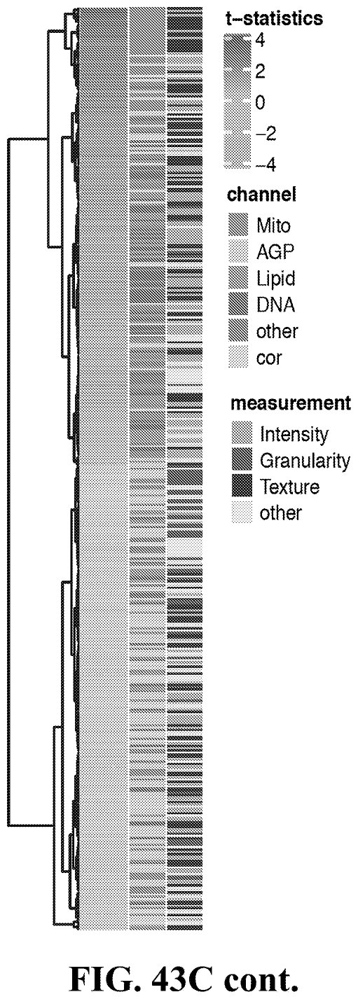

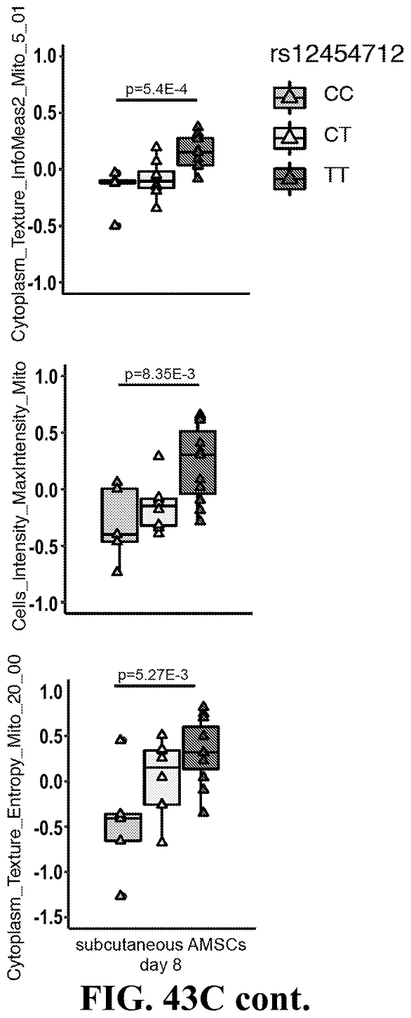

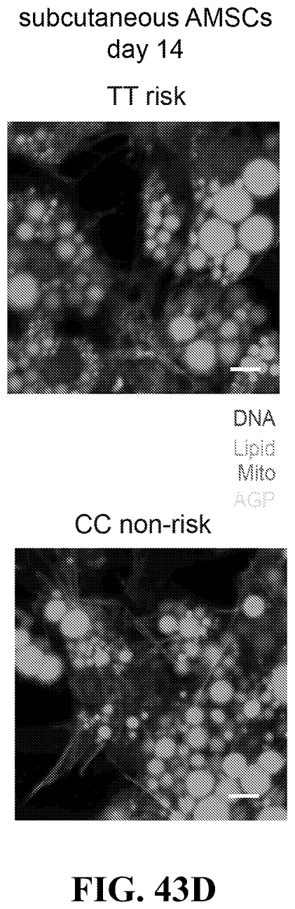

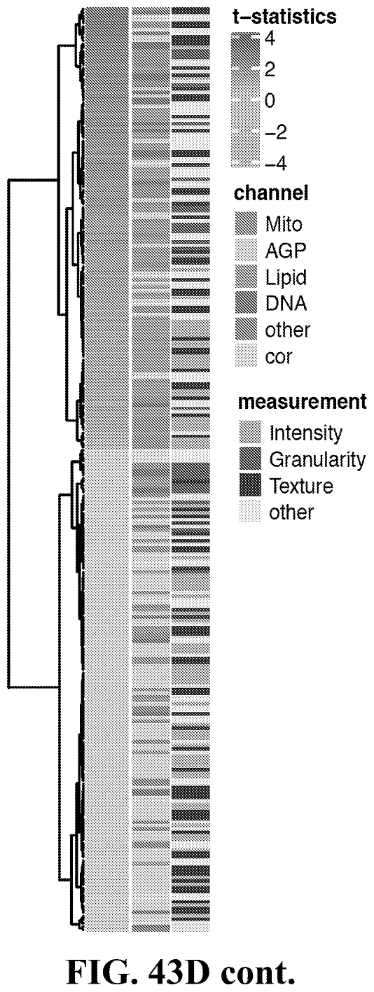

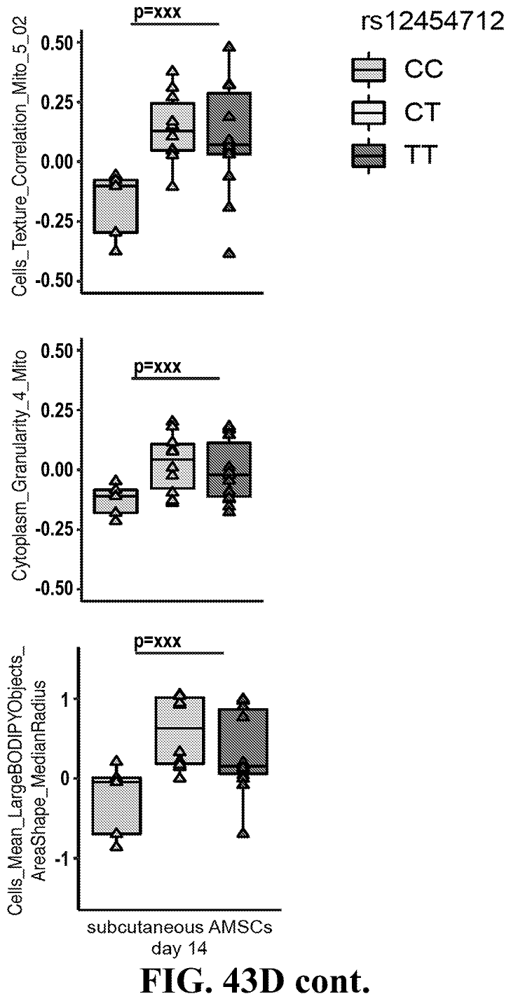

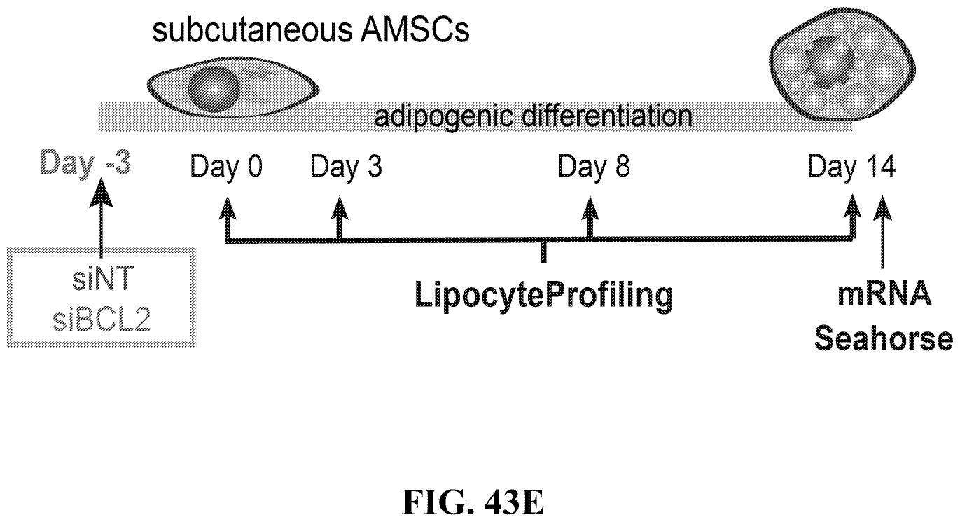

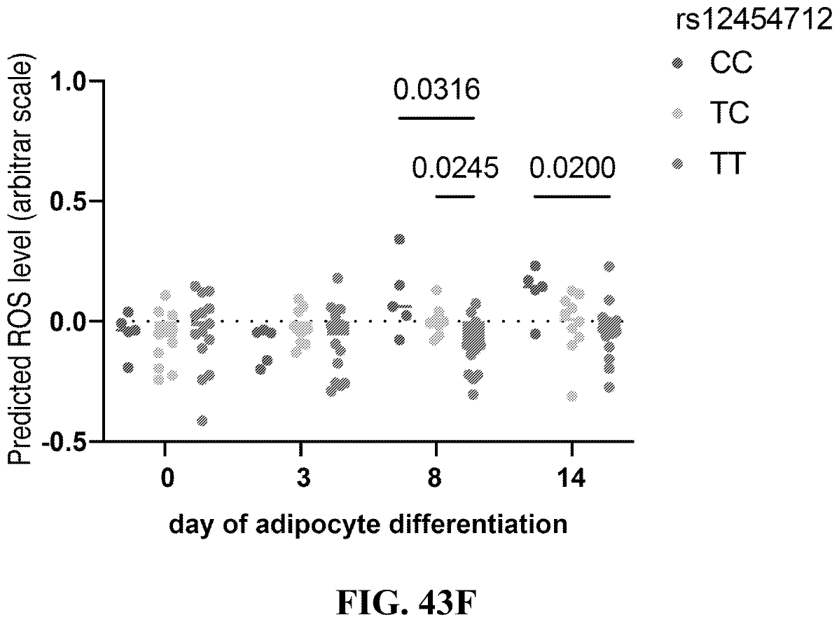

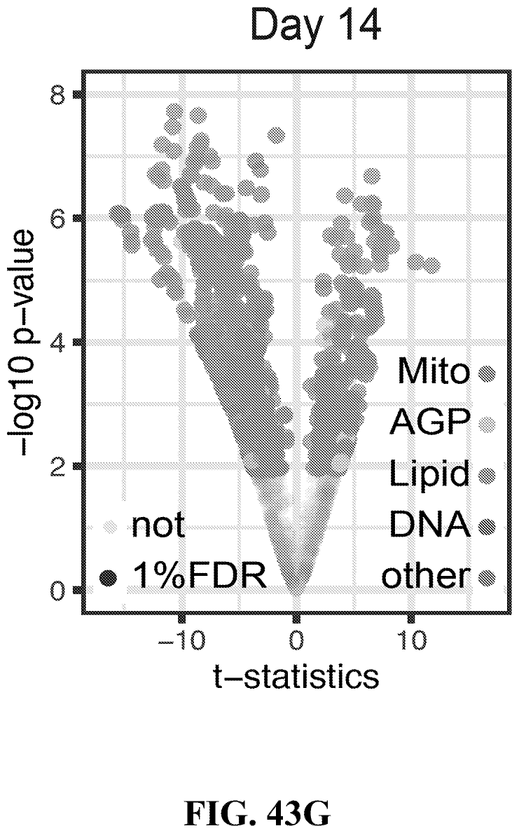

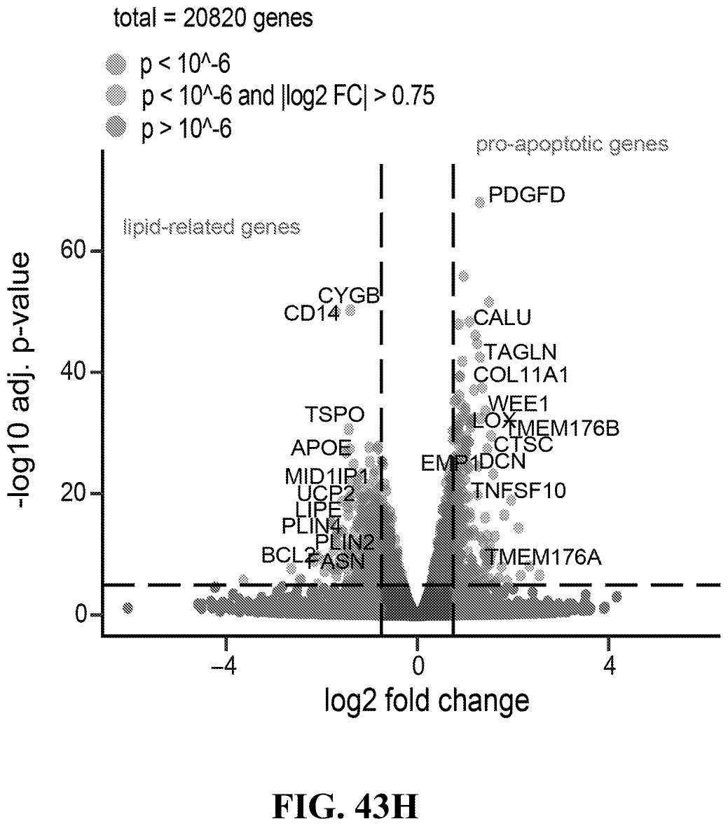

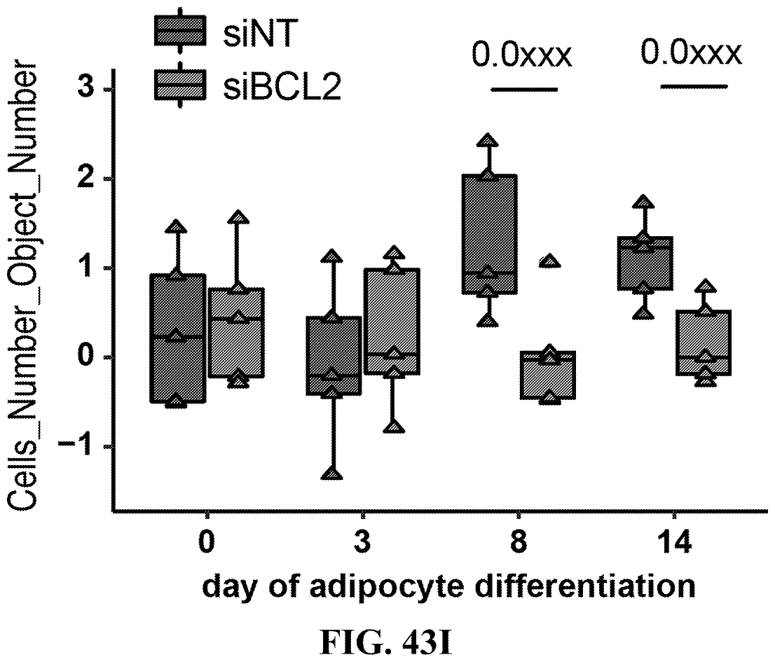

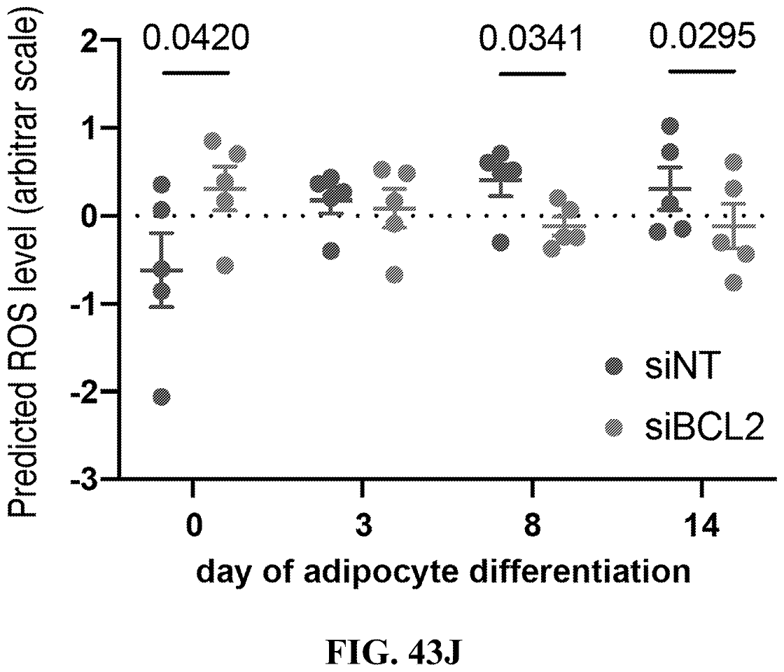

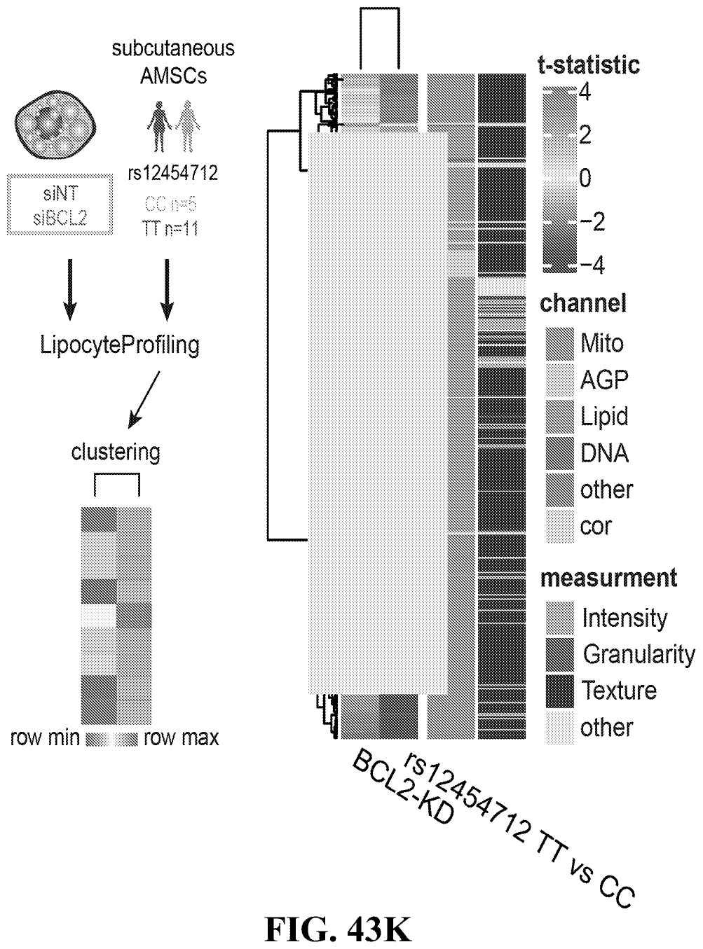

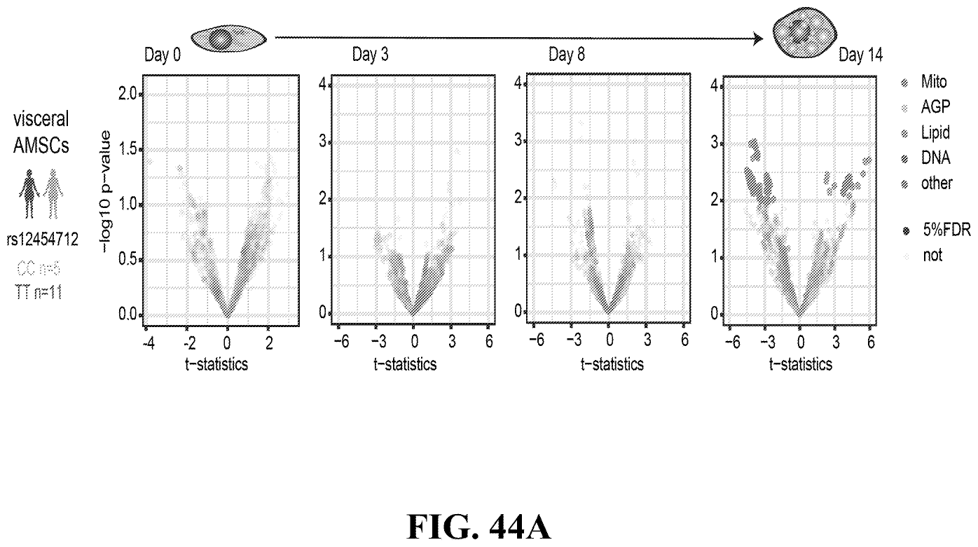

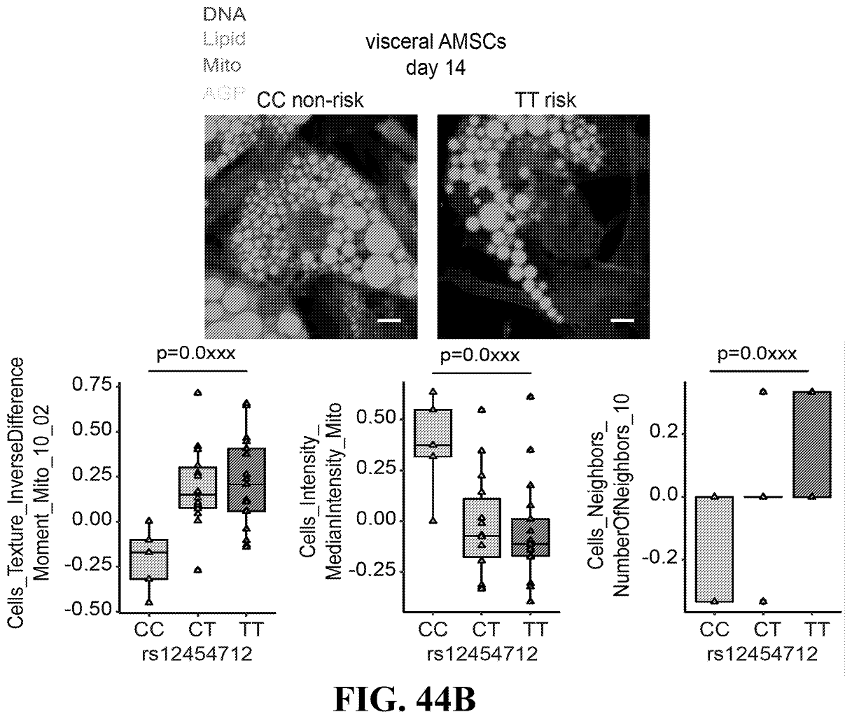

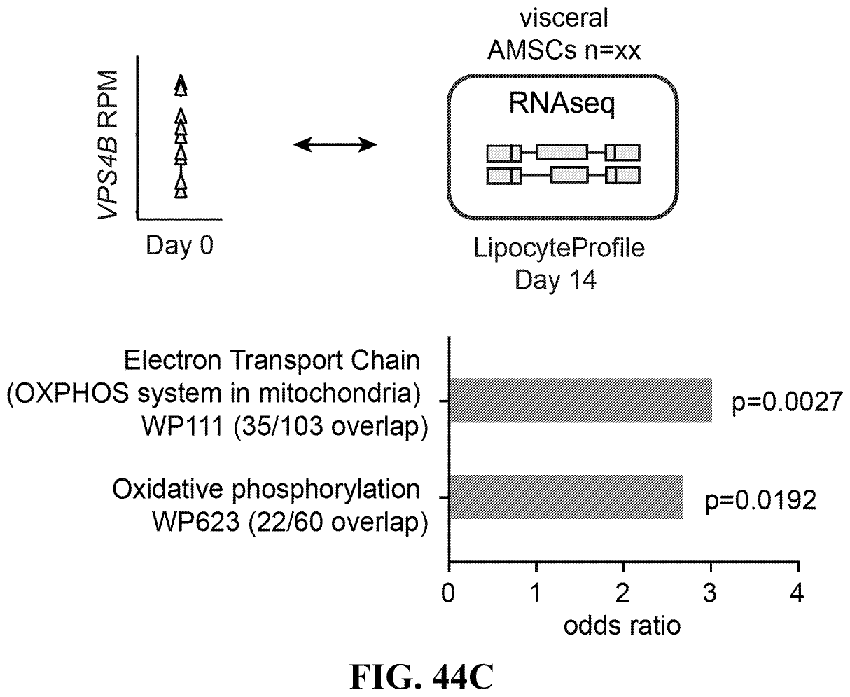

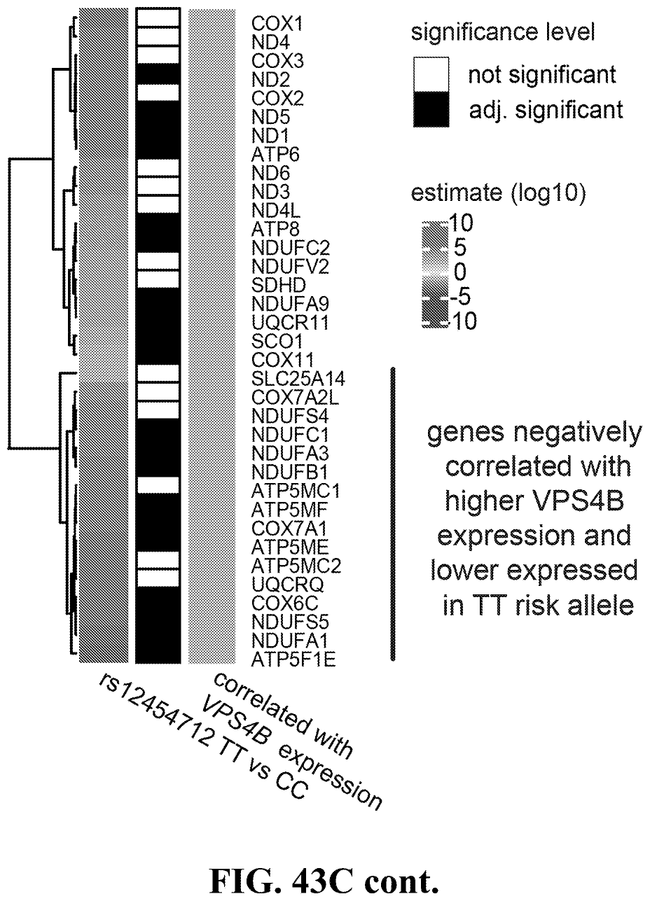

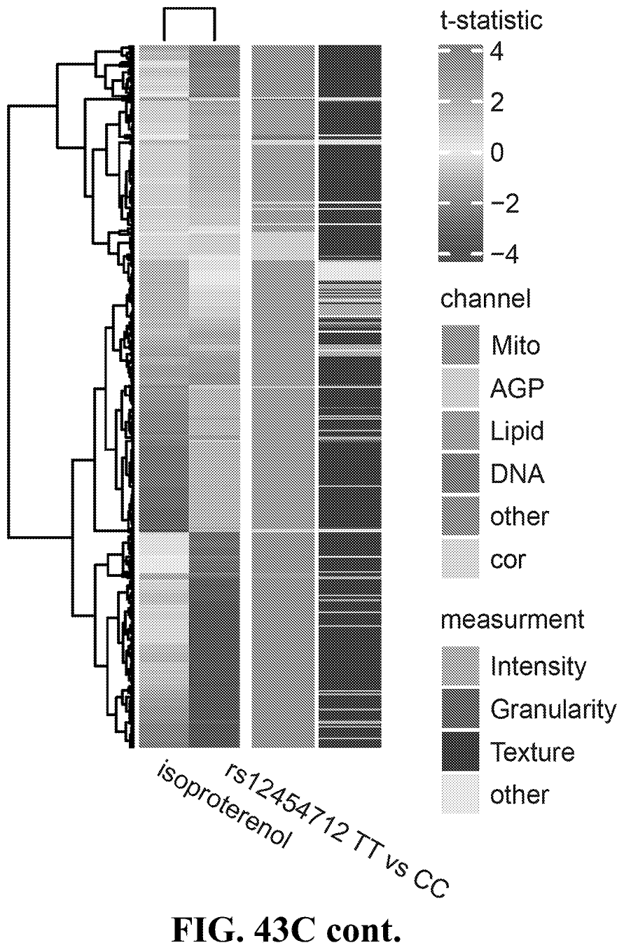

To identify possible functional consequences of rs12454712 in adipocytes, Applicants compared morphological and cellular profiles from TT and CC allele carriers in primary human subcutaneous and visceral AMSCs throughout adipocyte differentiation (FIG. 43A) using a recently established unbiased high content imaging assay, LipocyteProfiler (see, Example 1 and 3). In brief, LipocyteProfiler generates morphological profiles consisting of 3,005 features describing the structure, function and relationship between cellular organelles, namely AGP (actin cytoskeleton, Golgi and plasma membrane), Lipid (lipid droplets and cytoplasmic RNA), Mito (mitochondria) and DNA (nucleic-acid related phenotypes) (see, Example 1 and 3). Applicants found that the morphological profiles between rs12454712 TT and CC genotypes differed significantly in subcutaneous AMSCs at the later stages of adipocyte differentiation (day 8 and day 14) (FIG. 43B). At day 8 of differentiation, Applicants found 172 non-redundant significant features different between the haplotypes, most of which mapped to mitochondria-related features (FIG. 43E, Table 16). To visually confirm a haplotype-driven effect on predominantly mitochondrial features, Applicants generated images of the average cell of both haplotypes at day 8 representing the mean of all measurements and observed higher mitochondrial stain intensity in TT risk allele carriers compared to the CC non-risk (FIG. 43E). Three of the most significant features different between the alleles on day 8 were features informative for the structural appearance of mitochondria, as well as mitochondrial intensity features indicative of mitochondrial membrane potential. Mitochondria are the primary cellular source of reactive oxygen species (ROS), which play a critical role in cellular signalling and where levels above or beyond the physiological range are linked to altered cellular function and apoptosis (Suski et al. 2012). To ascertain that the rs12454712-associated mitochondrial profile shows similarities to a cellular profile of altered mitochondrial (ROS), Applicants next applied machine-learning (ML) based prediction of ROS from the recently developed CellHealth application (Way G P, Kost-Alimova M, Shibue T, et al. Predicting cell health phenotypes using image-based morphology profiling. Mol Biol Cell. 2021; 32 (9): 995-1005) to the profiles. Applicants found that at day 8 and 14, lipocyte profiles from TT risk allele carriers showed reduced ROS profiles (FIG. 43D), suggesting reduced intracellular signaling capacity in those cells.

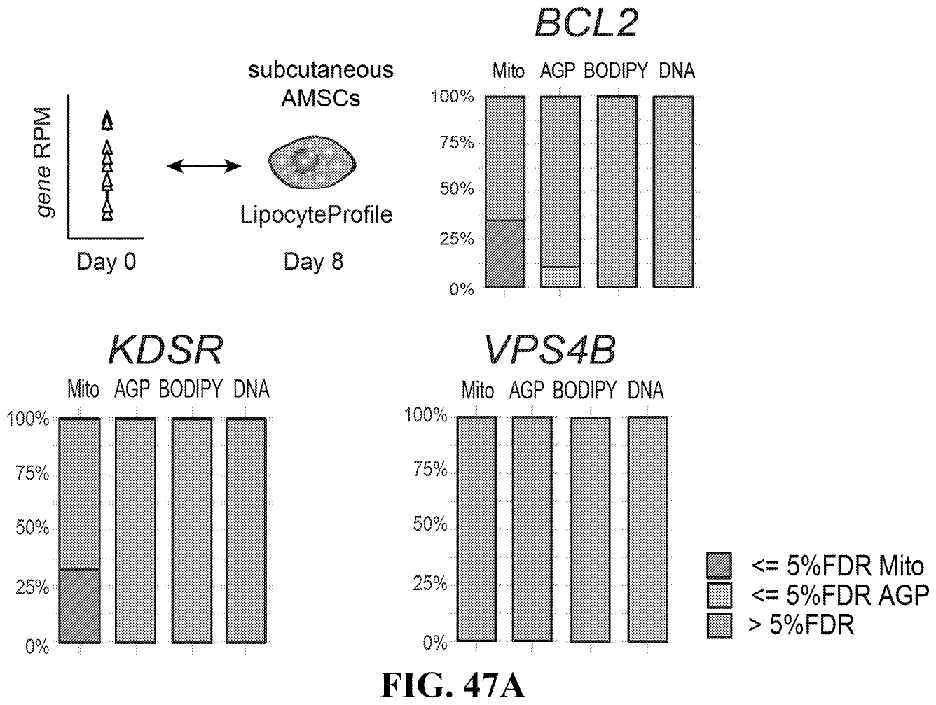

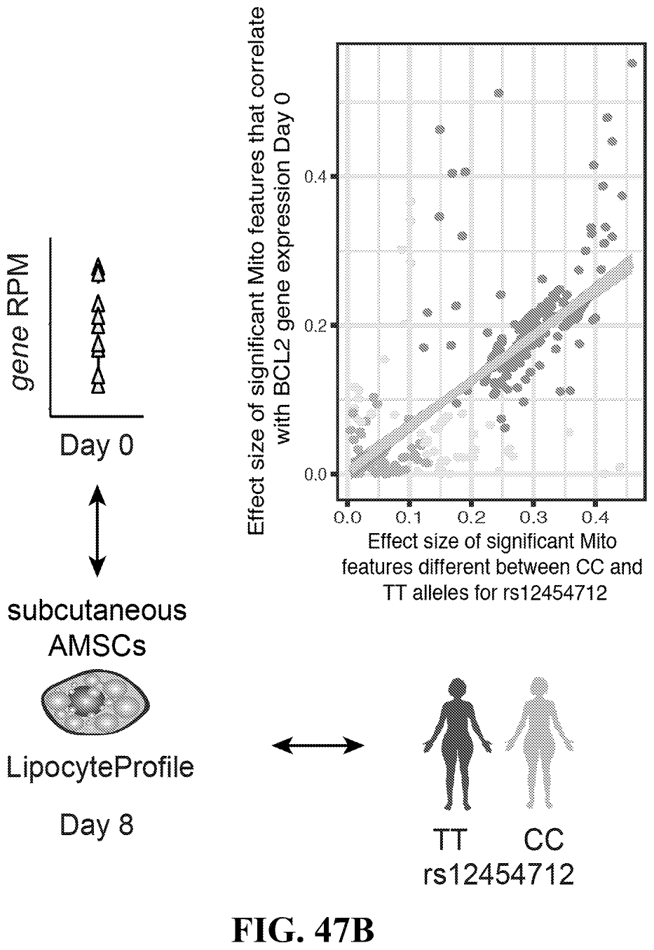

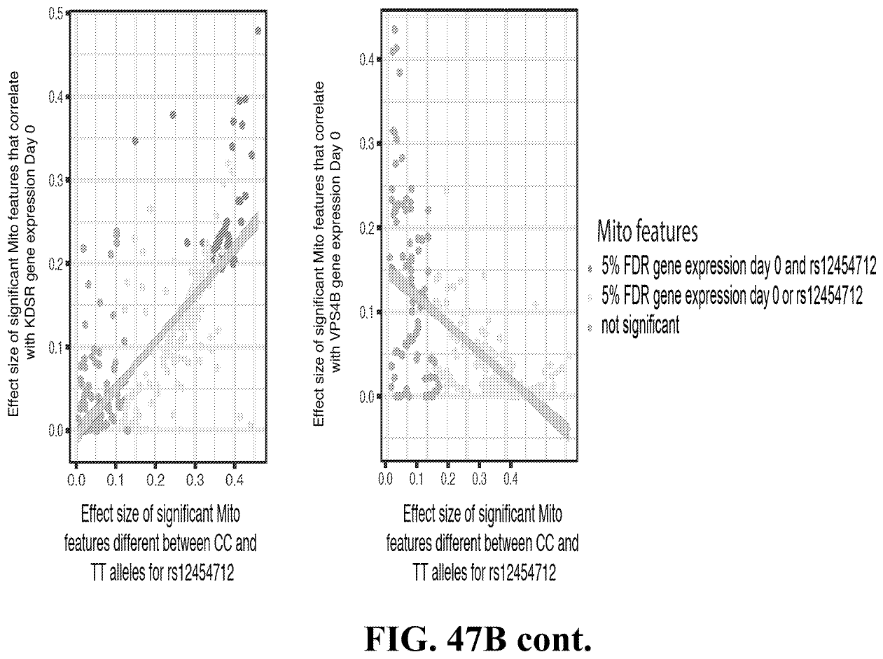

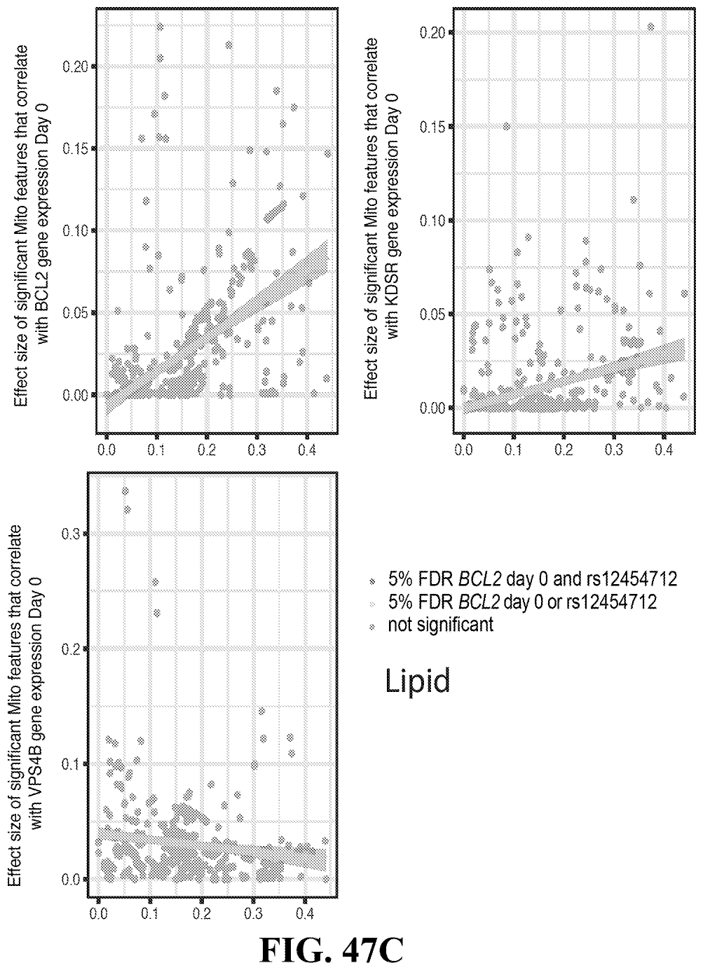

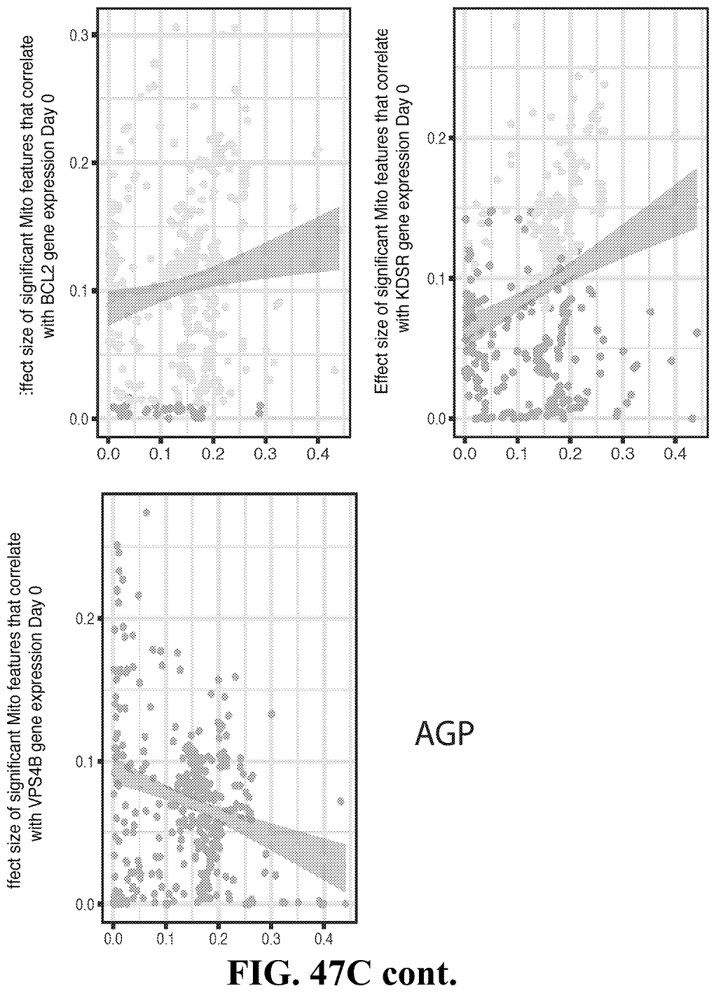

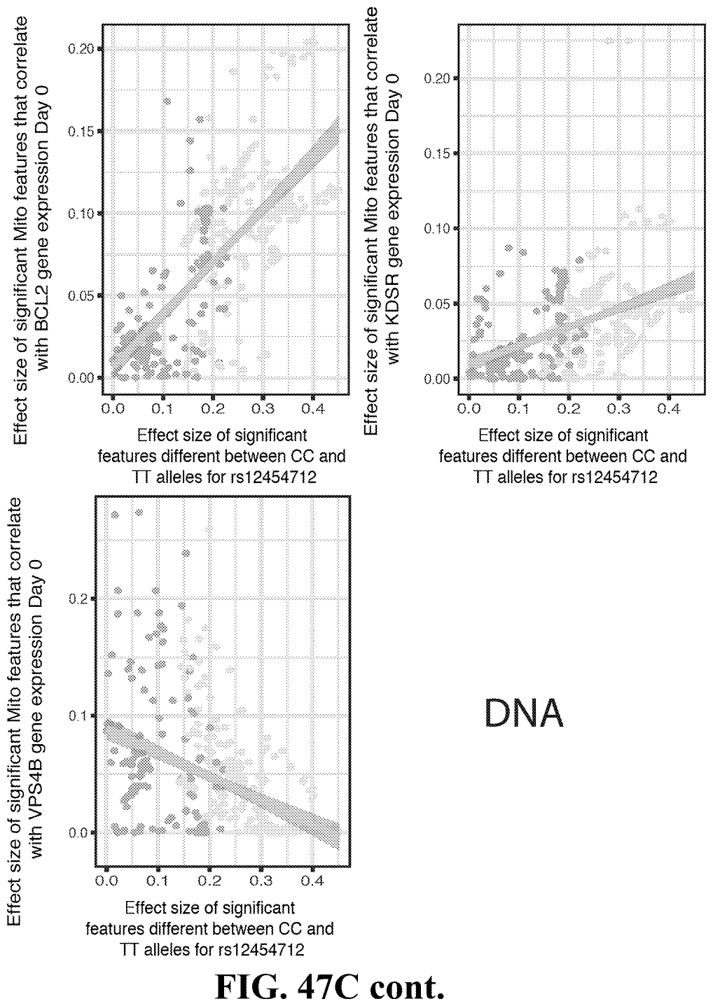

Intriguingly, although target gene expression changes were restricted to undifferentiated pre-adipocytes, the described haplotype-driven cellular consequences on mitochondria manifested in maturing adipocytes. To further assess the effect of target gene expression changes in adipocyte progenitors on function in mature adipocytes, Applicants next correlated BCL2, KDSR and VPS4B gene expression across 26 subcutaneous pre-adipocytes at day 0) (the cell stage in which Applicants see a genotype-driven effect on BCL2 and KDSR gene expression) with their morphological profile at day 8 (the cell stage where Applicants observed haplotype-driven effects on mitochondrial morphology and function). Applicants found that BCL2 and KDSR expression in undifferentiated AMSCs correlated with mitochondrial features at day 8 that resembled haplotype-driven effects when comparing TT with CC allele carriers at this time-point (FIG. 47A). Applicants then correlated effect sizes of LipocyteProfiler features driven by BCL2 and KDSR expression at day 0 with that of rs12454712 haplotype-driven effects on day 8 of differentiation and saw an overlap specifically of mitochondrial morphological features (FIG. 47B). This provides further evidence for BCL2 and KDSR expression in pre-adipocytes to be critical for the cellular haplotype-driven phenotype in maturing adipocytes, despite the absence of rs12454712-driven BCL2 or KDSR gene expression changes at this time point.

In terminally differentiated subcutaneous AMSCs (day 14), the TT risk haplotype manifested in a cellular profile that differed in 171 features from the CC non-risk haplotype. Those features spread across all four channels and across all feature classes (FIG. 43F, Table 17). Similar to day 8, adipocytes from TT risk allele carriers on day 14 showed mitochondrial stain patterns suggestive of a smoother appearance and higher number of small mitochondrial fragments (FIG. 43F), indicating that mitochondrial structure was altered in adipocytes from TT risk allele carriers in a manner similar to a profile of increased mitochondrial fragmentation. The TT risk allele further showed more and larger lipid droplets compared to non-risk allele carriers (FIG. 43F), which was also visible when comparing average cells between both haplotypes (FIG. 43F). Additionally, adipocytes from TT allele carriers had smaller nuclei (Nuclei_AreaShape_MedianRadius), fewer neighbours (Cells_Neighbors_NumberOfNeighbors_Adjacent), and a more condensed cytoplasm (Cytoplasm AreaShape Compactness) (Table 17), all of which are known morphological characteristics of apoptotic cells. Indeed, increased mitochondrial membrane potential and mitochondrial fission/fragmentation are early and fundamental hallmarks of apoptosis, a process progressing into a distinct set of physical changes involving the cytoplasm, nucleus, and plasma membrane (Ly et al. 2003). In the cytoplasm, apoptosis is characterized by the accumulation of cytoplasmic lipid droplets composed largely of neutral lipids (Boren and Brindle 2012). In the nucleus, chromatin condenses and is fragmented by endonucleases. In the plasma membrane, cell junctions are disintegrated, and cells eventually break up. Finally, apoptotic cells round up, lose contact with neighboring cells and shrink (Ly et al. 2003), physical changes that we observe in morphological profiles of TT risk adipocytes (Table 17).

To identify possible, Applicants generated a network consistent of all genes associated with haplotype-driven differential features (<5% FDR) at day 8 based on a linear regression model of LipocyteProfiler-derived features and transcriptome-wide gene expression data of subcutaneous differentiated adipocytes (day 14). Applicants identified 2539 genes that associated significantly (FDR 0.1%) with the morphological and cellular profile of the rs12454712 genotype in subcutaneous adipocytes. The identified genes were significantly enriched for pathways characterizing fatty acid catabolic process (GO: 0009062) and apoptosis (GO): 1900117, 1900118, 1900119) (Table 18). Together, both morphological profiling and gene expression results point towards rs17454712 mediating apoptotic and lipid degradation processes.

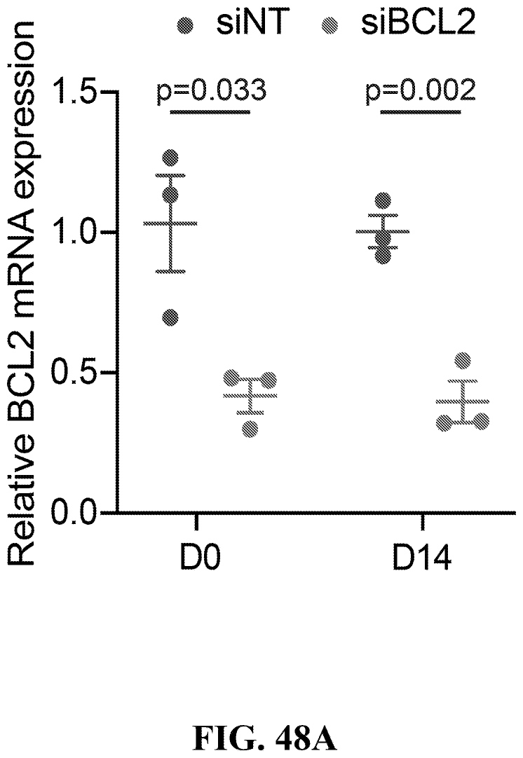

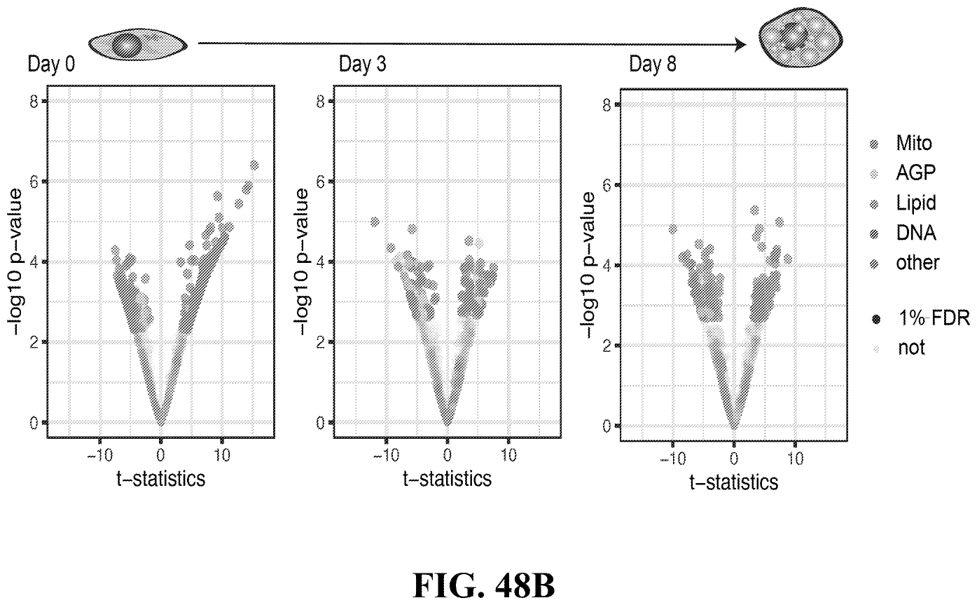

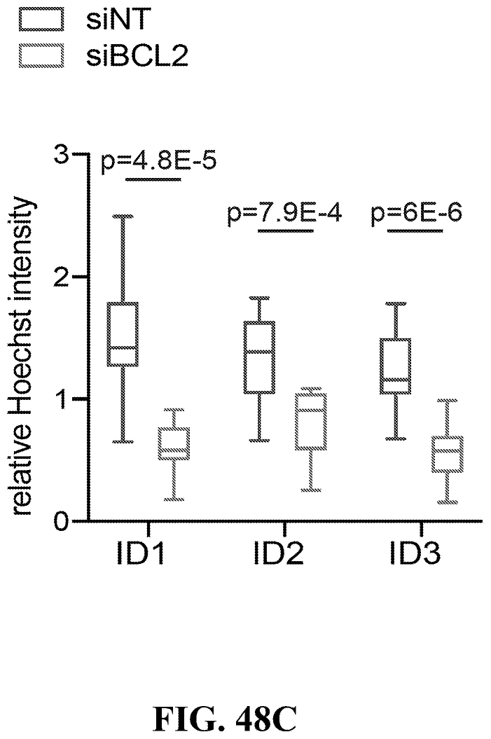

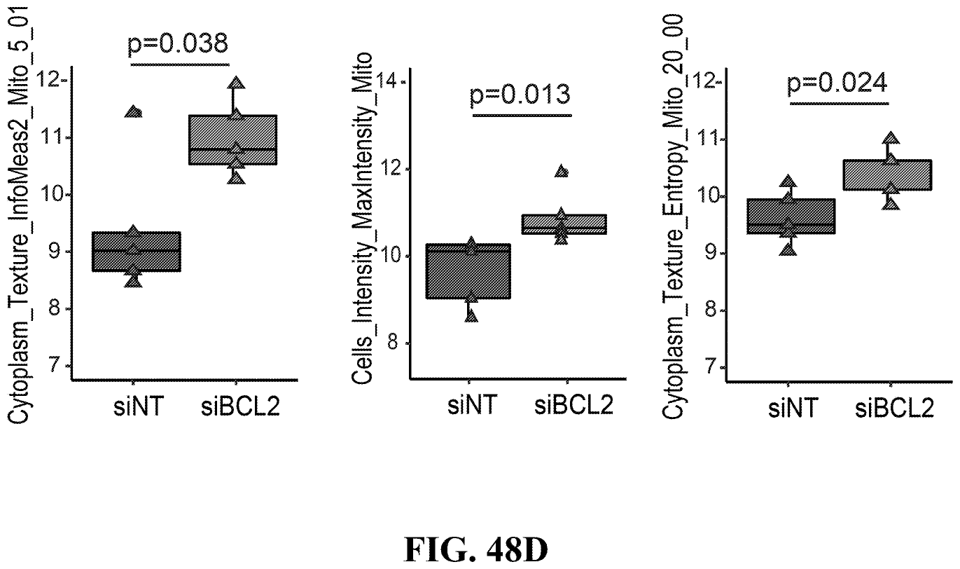

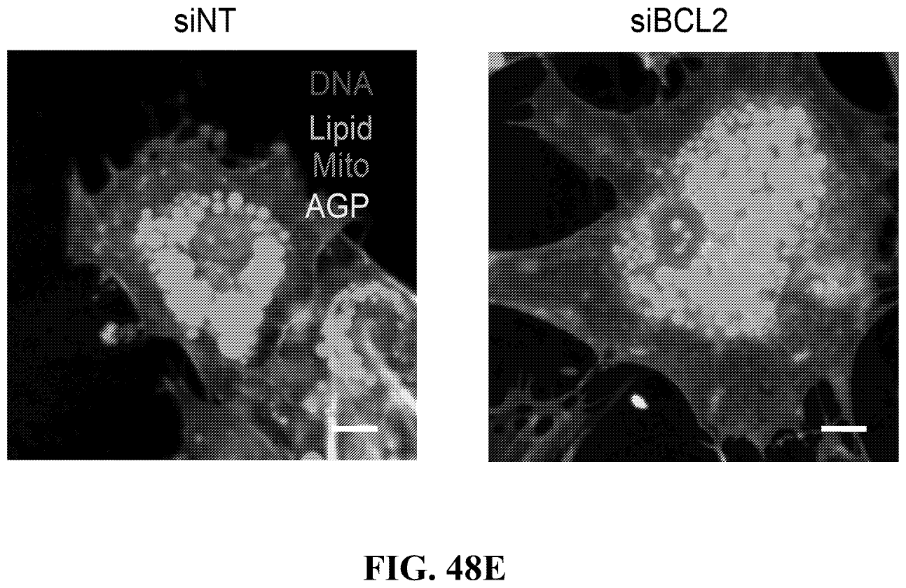

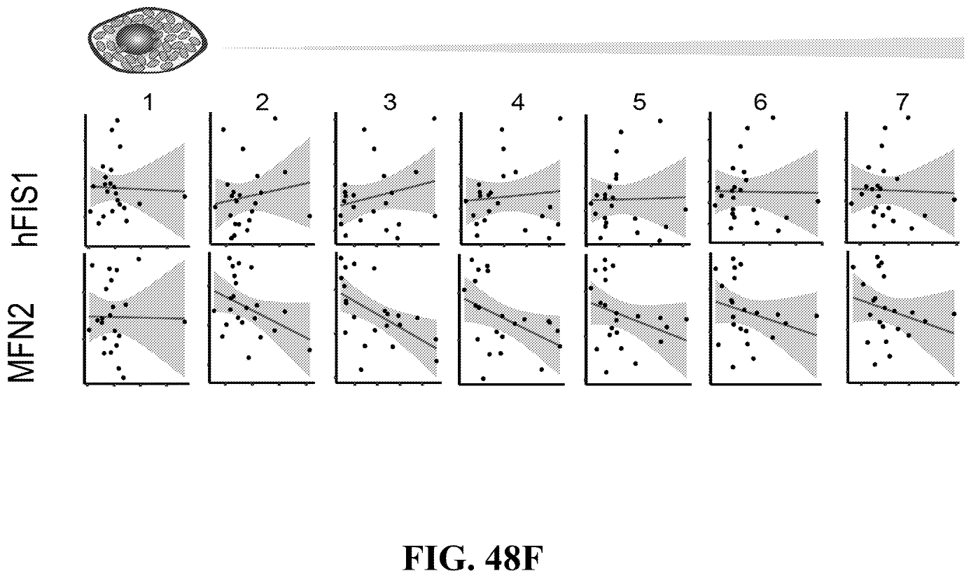

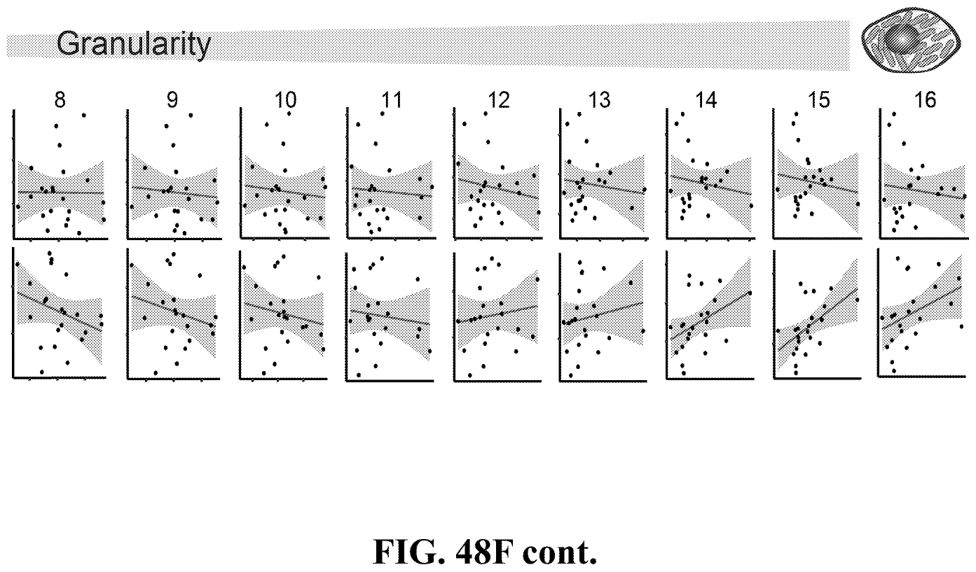

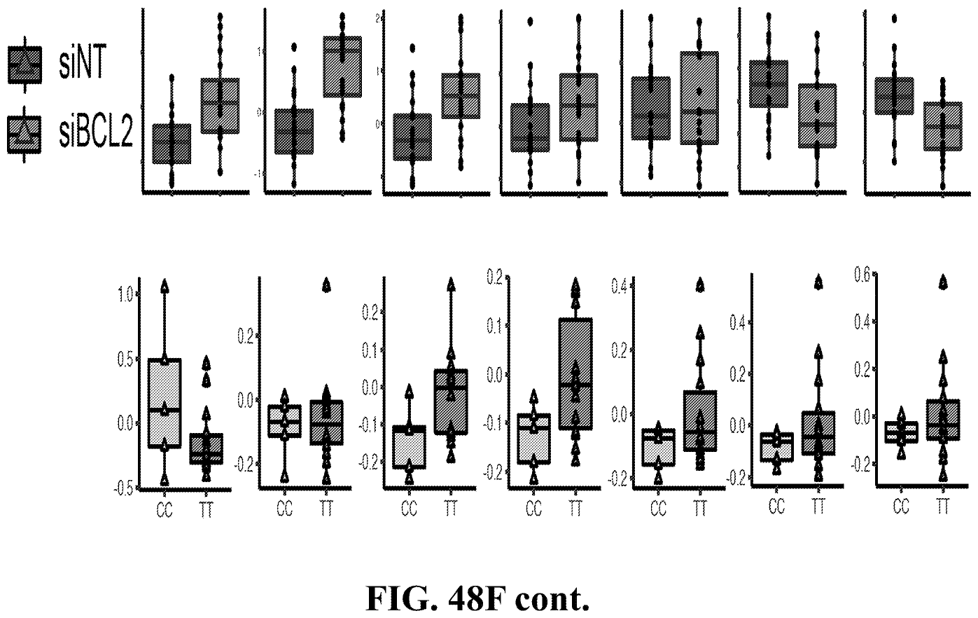

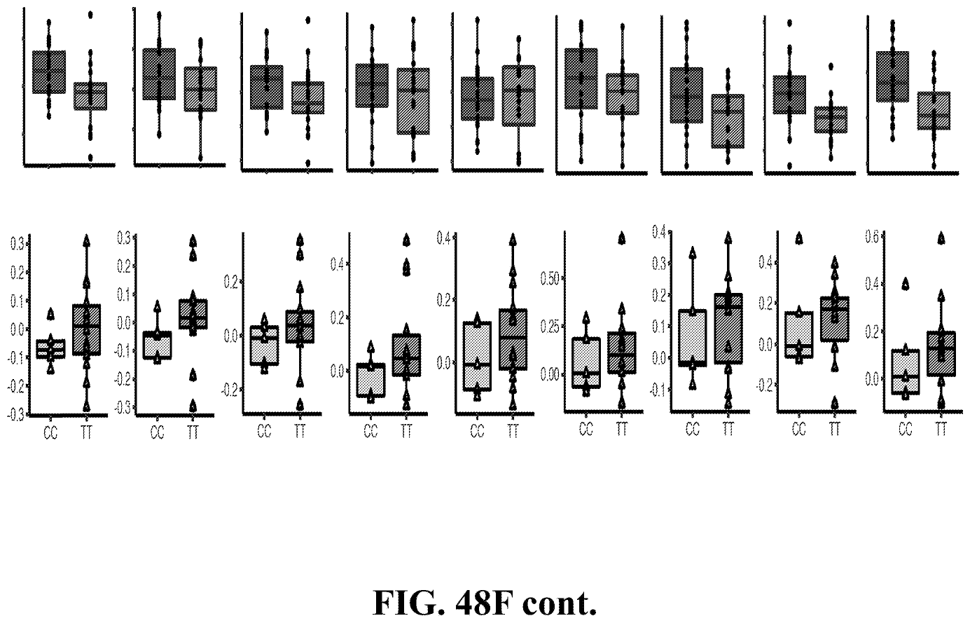

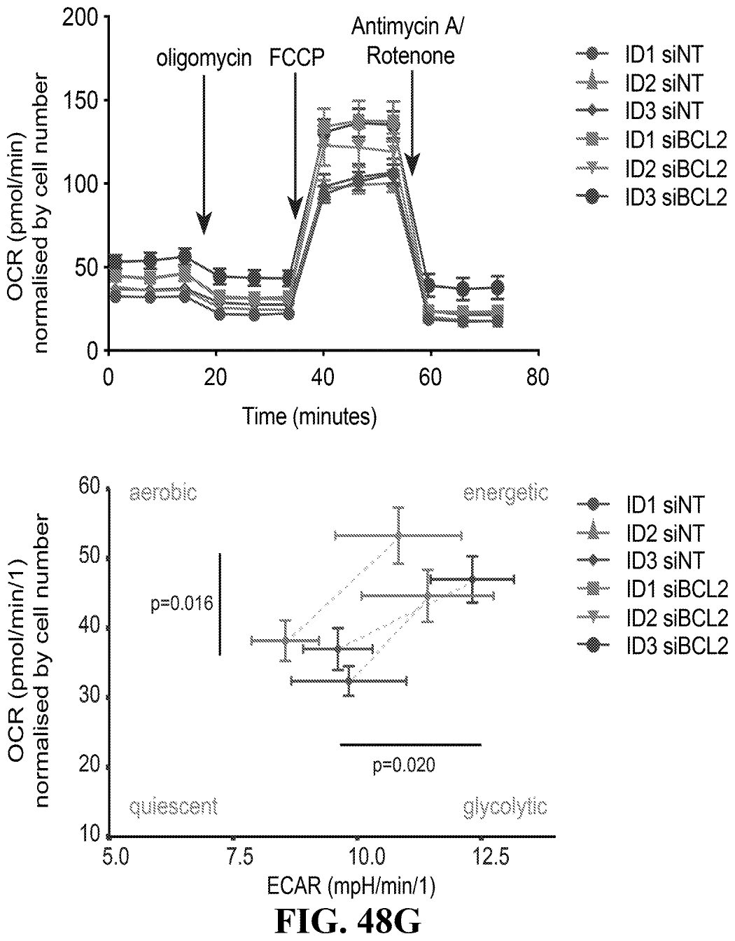

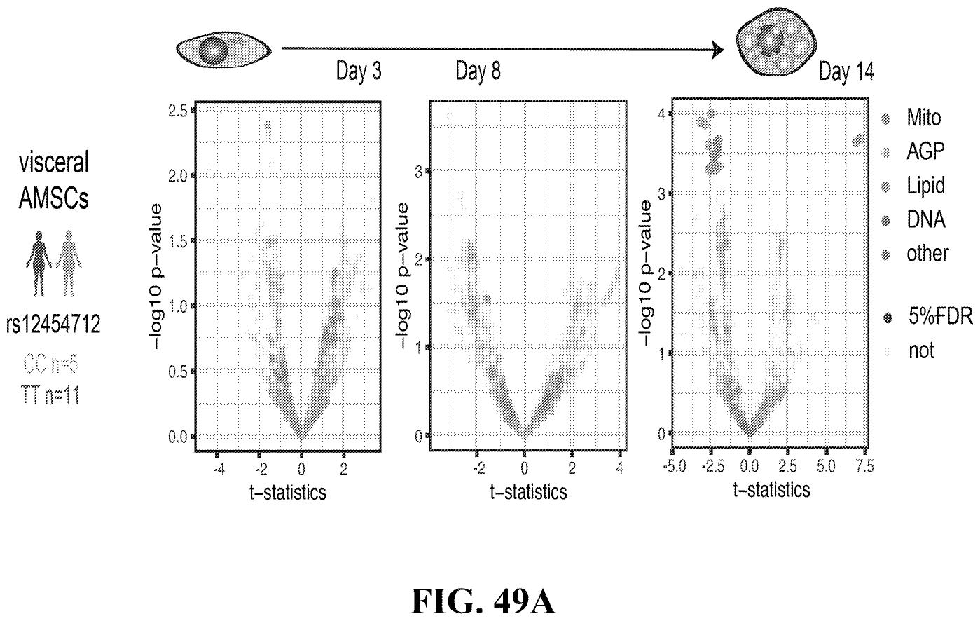

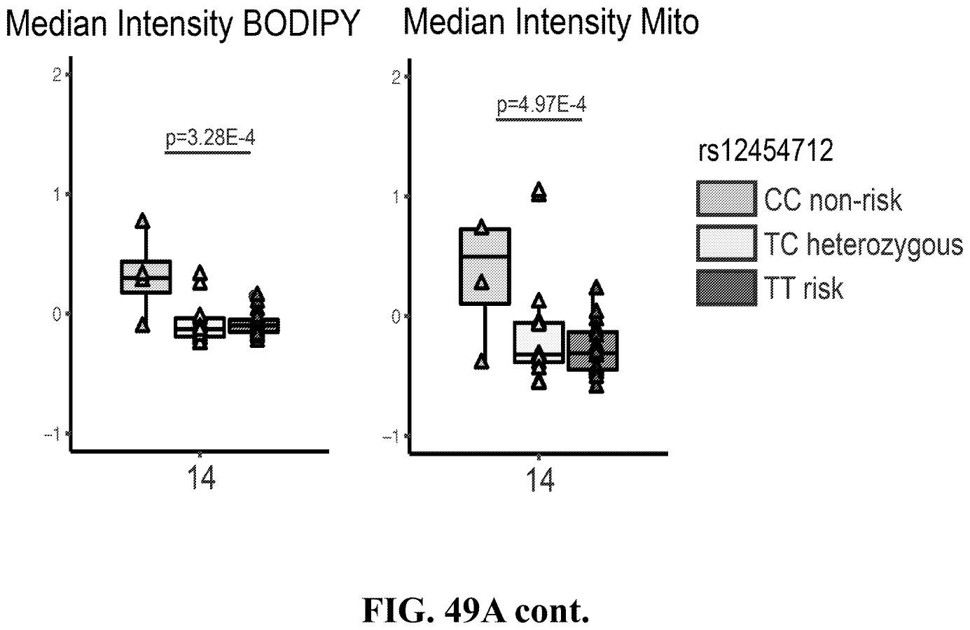

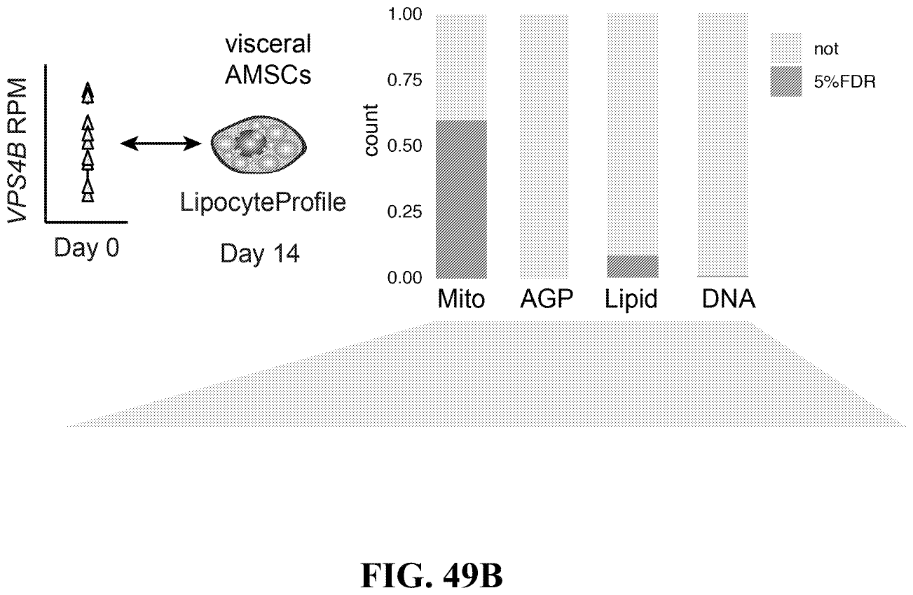

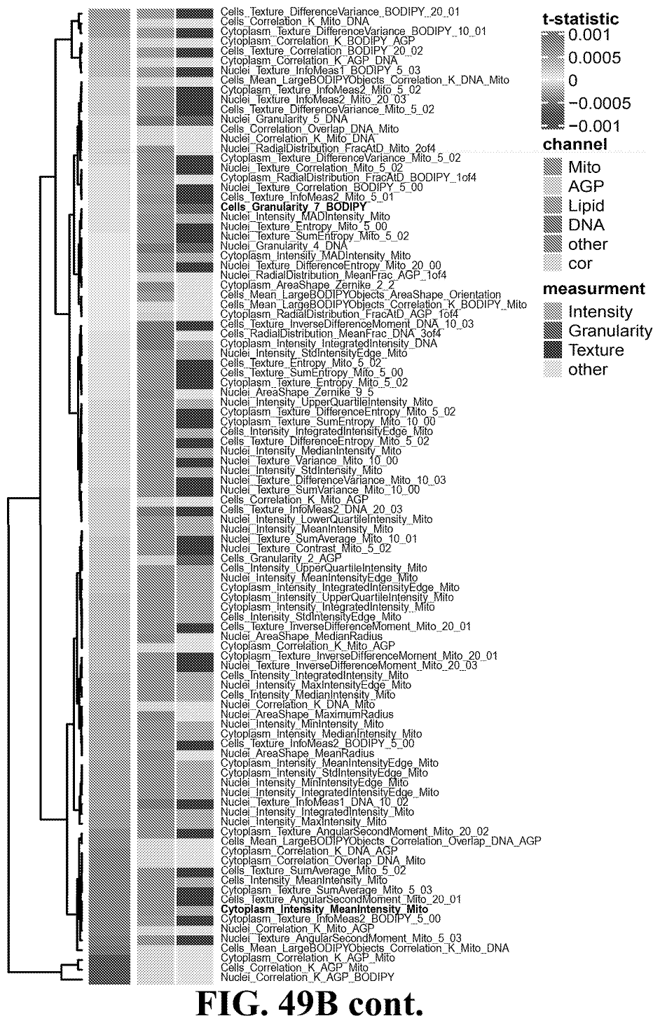

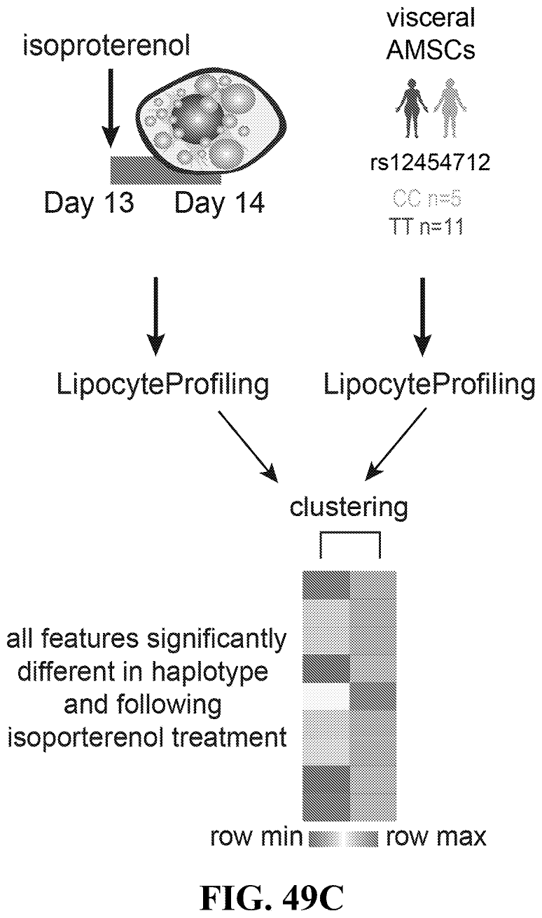

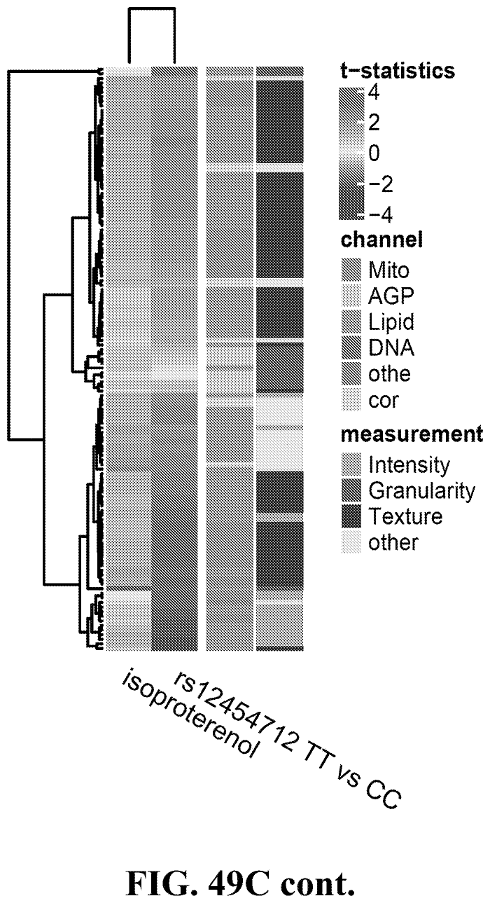

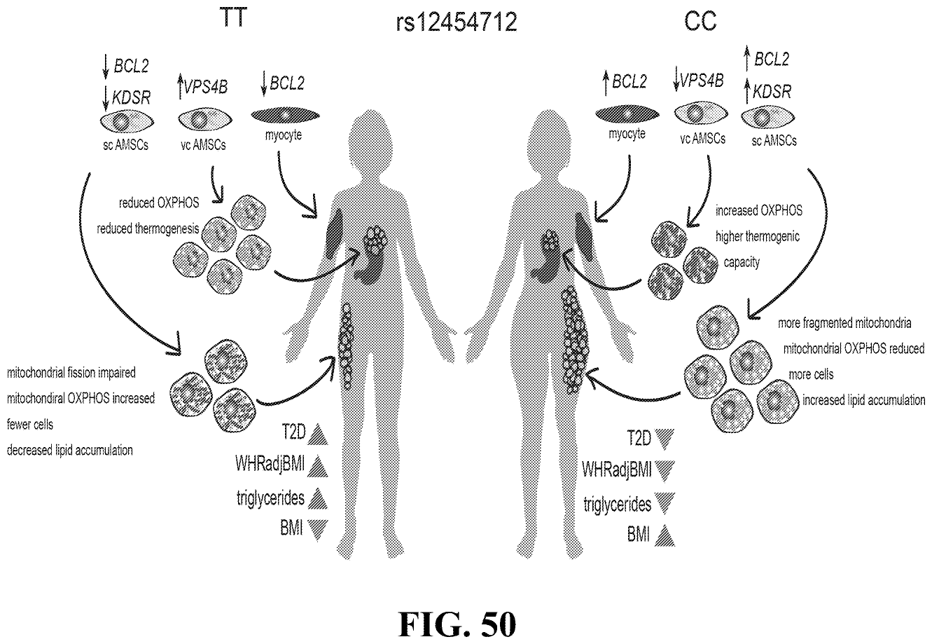

To further validate whether the rs12454712-associated morphological profile resembles cellular signatures that Applicants would expect to see in a state of increased apoptosis, Applicants next generated a cellular reference profile of apoptosis by silencing the well-known anti-apoptotic gene BCL2 using siRNA (˜60% knockdown efficiency; FIG. 48A) in subcutaneous AMSCs from five normal-weight female individuals. Applicants assessed cell number, cell morphology (LipocyteProfiler) and mitochondrial respiration using the Seahorse Bioflux Analyser (FIG. 48G). By day 14, BCL2-KD) reduced cell numbers by ˜50% as assessed using Hoechst intensity (FIG. 48C). Interestingly, the pro-apoptotic consequences of BCL2 loss were restricted to mature adipocytes (days 8 and 14), as there was no difference in cell numbers in AMSCs before induction of adipogenesis and in early differentiation (days 0) and 3) (FIG. 43J). This is in line with previous studies reporting anti-apoptotic functions of BCL2 family members are restricted to differentiating cells, and not detected in mesenchymal stem cells (Oliver et al. 2011). When comparing LipocyteProfiles between BCL2-KD and non-targeting control AMSCs, Applicants found that BCL2-KD alters cellular profiles throughout differentiation, with the strongest effect in day 14 adipocytes where mitochondrial and lipid-related features predominate the BCL2-KD-mediated LipocyteProfiles (FIG. 43H, Table 19). Comparing the individual Mito texture and intensity features that Applicants previously examined in rs12454712 haplotype shows that BCL2-KD increases Mito texture and intensity (FIG. 48D), resembling the TT risk haplotype. Additionally, Mito granularity features were increased in BCL2-KO adipocytes specifically for the smaller, and decreased for the larger granularity measurements (FIG. 48F), indicating more fragmented mitochondria in BCL2-KO adipocytes compared to siNT-treated cells. This mitochondrial fragmented phenotype is consistent with mitochondrial fission, as gene expression of hFis, a mitochondrial fission gene, correlated negatively with larger granularity measures across adipocytes from 26 individuals. Gene expression of MFN2, a mitochondrial fusion gene, correlated negatively with smaller and positively with larger granularity measures, suggesting that mitochondrial fragmentation phenotype observed in adipocytes from TT risk allele carriers and BCL2-KO adipocytes is indicative of increased mitochondrial fission. Transcriptome-wide gene expression of BCL2 knockdown versus control showed that in subcutaneous AMSCs at day 14 pro-apoptotic genes (e.g. TNFSF10, DCN, CALU, TAGEN) were significantly upregulated, whereas genes related to lipid metabolism (e.g. LIPE, PLIN4, FASN and APOE) were downregulated (FIG. 43I; Table 20), similar to what Applicants had observed for the rs12454712 risk allele.

To test if siBCL2-induced cellular changes are associated with altered mitochondrial ROS in a similar fashion as Applicants observed for the rs12454712 genotype, Applicants next applied the same ML-based approach we used to predict ROS levels in TT versus CC allele carriers now in LipocyteProfiles from siBCL2-treated cells. Applicants found that at day 8 and 14 of adipocyte differentiation, adipocytes of BCL2-KD had reduced predicted ROS levels (FIG. 43K), mimicking the rs12454712 risk allele.

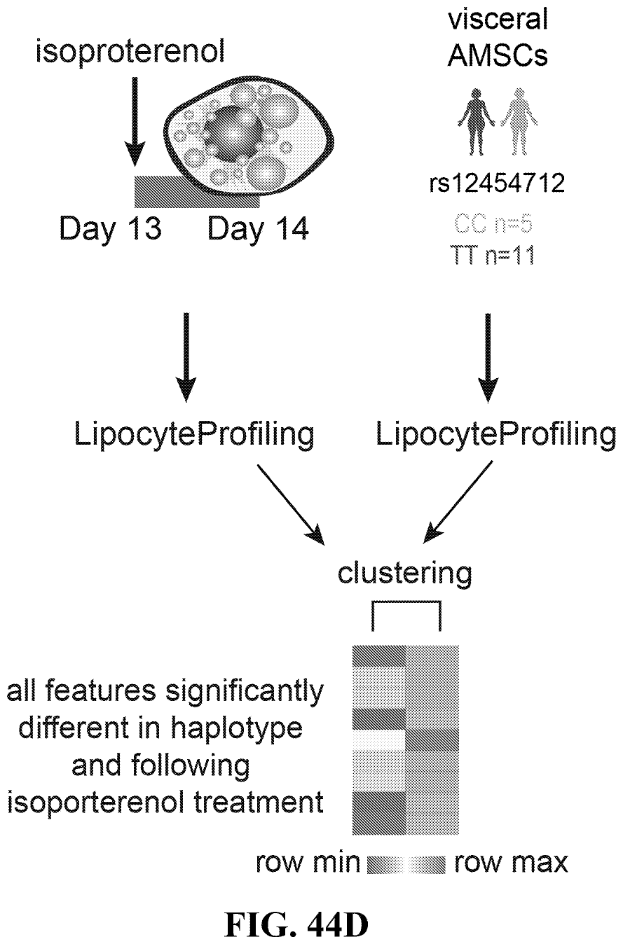

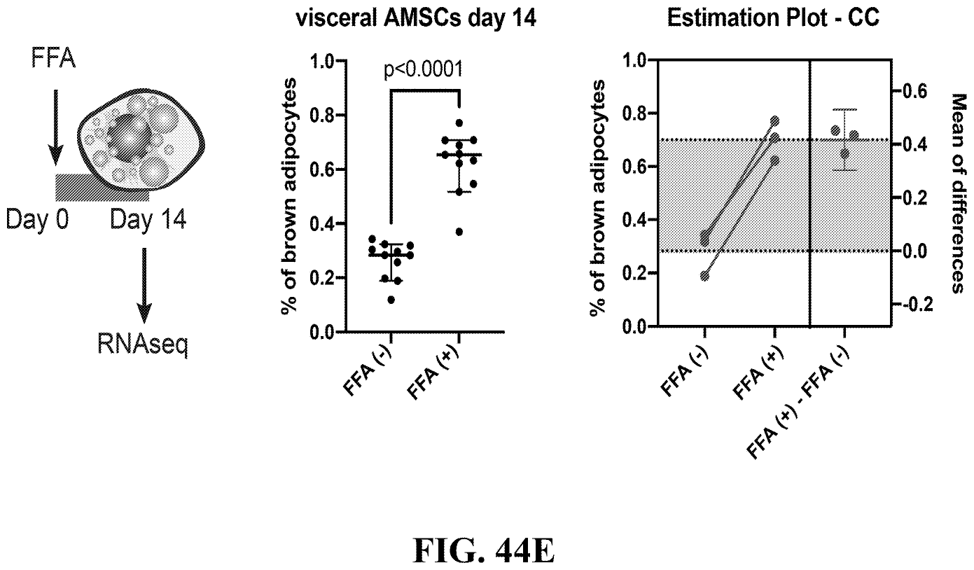

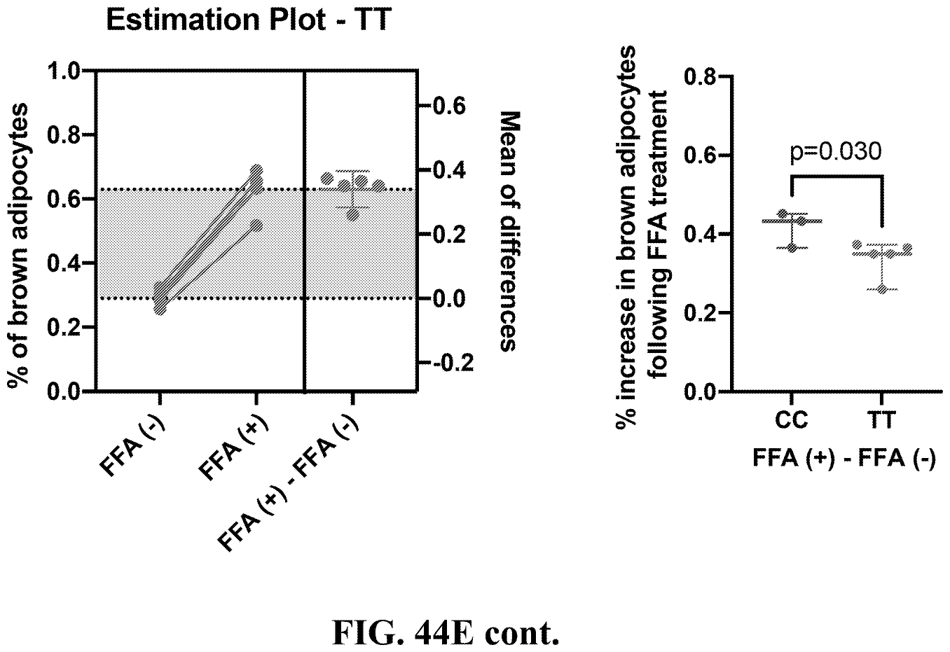

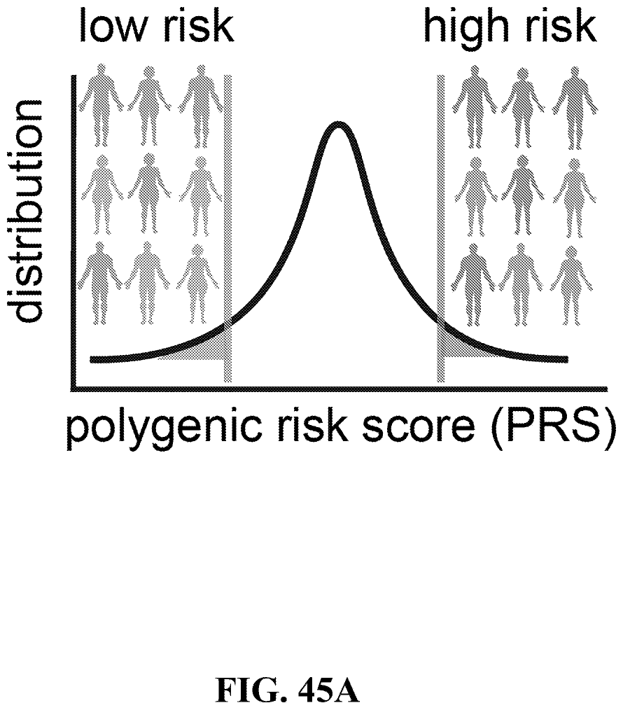

Applicants next investigated whether these BCL2-KD-induced morphological changes translate into altered mitochondrial respiration in a mitochondrial stress test using the Seahorse Bioflux Analyser. BCL2-KD increased oxygen consumption rate (OCR) and extracellular acidification rate (ECR) (FIG. 48G), revealing a more energetic profile in BCL2-KD adipocytes compared to control (FIG. 44G). This is consistent with a recent study reporting an unexpected increase in maximal respiration in subcutaneous adipocytes of metabollically unhealthy obese subjects compared to metabolically healthy obese (Böhm et al. 2020), suggesting that mitochondrial impairment and a possible increased mitochondrial permeability could be an underlying mechanism in subcutaneous adipocytes of insulin resistant individuals. Together, these results suggest that BCL2-KD induces mitochondrial fission, reduces cell number, and increases OCR and extracellular acidification rate. These cellular consequences are consistent with haplotype-driven effects of the TT risk allele and are consistent with a phenotype of altered ROS production and initiation of the apoptotic program. Together, Applicants showed that the TT risk allele for rs12454712 reduces BCL2 and KDSR expression in preadipocytes which leads first to altered mitochondrial structure and function which then progresses into a perturbed cellular and morphological profile in maturing adipocytes resembling apoptosis.

Due to their apoptotic properties, BCL2 inhibitors are currently used in the clinic for chronic lymphocytic leukemia and small lymphocytic lymphoma. Importantly, pharmacological inhibition of BCL2 using venetoclax has been reported to cause hyperglycemia in 16% of patients and severe hyperglycemia in 5% in a 1 year follow up clinical trial (Roberts et al. 2016), as well as loss of body weight in 11-13% of cases, indicating that BCL2 inhibition can lead to systemic metabolic adverse effects including reduced insulin sensitivity.

In visceral AMSCs, Applicants observed a rs12454712 genotype-driven effect on predominantly mitochondrial-associated morphological features at day 14 of differentiation (FIG. 44A; Table 21). More specifically, Applicants observed higher cell number, decreased mitochondrial stain intensity (indicative of mitochondrial membrane potential), and structural differences based on mitochondrial stain intensities in TT risk allele carriers (FIG. 44B). Applicants confirmed these LipocyteProfiler-based findings visually by comparing average cells between both haplotypes in visceral adipocytes at day 14 (FIG. 44B). To assess whether these haplotype-driven cellular changes in visceral adipocytes could be driven by VSPB4 target gene expression changes in visceral pre-adipocytes, Applicants correlated VSPB4 expression at day 0 (where Applicants observed genotype-mediated effect on target gene expression) with both transcriptome-wide gene expression and LipocyteProfiler-derived features in visceral adipocytes on day 14 of differentiation (the time-point where Applicants see morphological consequences of the risk haplotype) as previously described (see FIG. 47). Applicants found that both analyses pointed towards a role for VPS4B expression in preadipocytes to mediating mitochondrial mechanisms in mature adipocytes. Specifically, VSPB4 expression in preadipocytes associated with genes enriched for OXPHOS pathways (FIG. 44C) and for features mapping to the mitochondria channel (FIG. 44C). Applicants next validated VPS4B as the potential target gene at the rs12454712 locus by comparing expression of the 35 OXPHOS genes significantly associated with VPS4B expression at day 0, between TT and CC allele carriers. Applicants confirmed that gene regulation of TT vs. CC allele carriers mimic the effect of VPS4B expression at day 0 on OXPHOS gene enrichment at day 14 (FIG. 44C). Applicants could not observe a similar association for the other potential target genes BCL2 and KDSR.

Specifically, higher expression of VSPB4 (as seen in pre-adipocytes of the TT risk allele) correlated negatively with Mito intensity and texture, but positively with a feature describing overlap between mitochondrial and lipid stain, suggesting a profile of reduced mitochondrial membrane potential and higher colocalization of mitochondria to lipid droplets. This cellular profile is indicative of altered thermogenesis, as mitochondria are anchored to lipid droplets during ATP production and lipid droplet expansion but dissociate during browning-induced fatty acid oxidation (Benador et al. 2018). Disruption of this process has been linked to reduced insulin sensitivity in adipose tissue.

To assess whether visceral AMSCs from TT risk allele carriers resemble morphological profiles that Applicants would expect in a state of reduced thermogenic capacity, Applicants next compared rs12454712-driven morphological signatures with that of isoproterenol-treated visceral AMSCs at day 14 (see Examples 1 and 3). Isoproterenol is an adrenergic agonist known to induce adipocyte browning and increase thermogenic capacity. Applicants found features that are significantly different following isoproterenol treatment and/or between the rs12454712 haplotypes (<FDR 5%). Those features mapped predominantly to the lipid and mitochondria channels (FIG. 44C) and overlapped in their direction of effect, indicating that rs12454712 affects similar cellular programs than isoproterenol treatment. As an orthogonal approach, Applicants next compared the percentage of thermogenesis active adipocytes between haplotypes by differentiating visceral AMSCs with free fatty acids (FFA) (oleic and linoleic acid; see methods), which are another known stimulus of thermogenesis in adipocytes. Using BATLAS, Applicants confirmed that FFA treatment indeed increased the percentage of thermogenic active adipocytes (FIG. 44E). However, TT risk allele carriers show lower percentage of thermogenesis active adipocytes compared to CC allele carriers following FFA treatment, despite similar basal levels, suggesting that visceral AMSCs from TT risk allele carriers show a blunted response to thermogenic stimulus. This supports a model in which elevated VSP4B expression in TT risk allele carriers causes reduced mitochondrial thermogenic capacity in mature adipocytes. Together, these data suggest an adipose depot-dependent effect, in which the TT risk allele associates with distinct cellular signatures in adipocytes from subcutaneous and visceral adipose tissue.

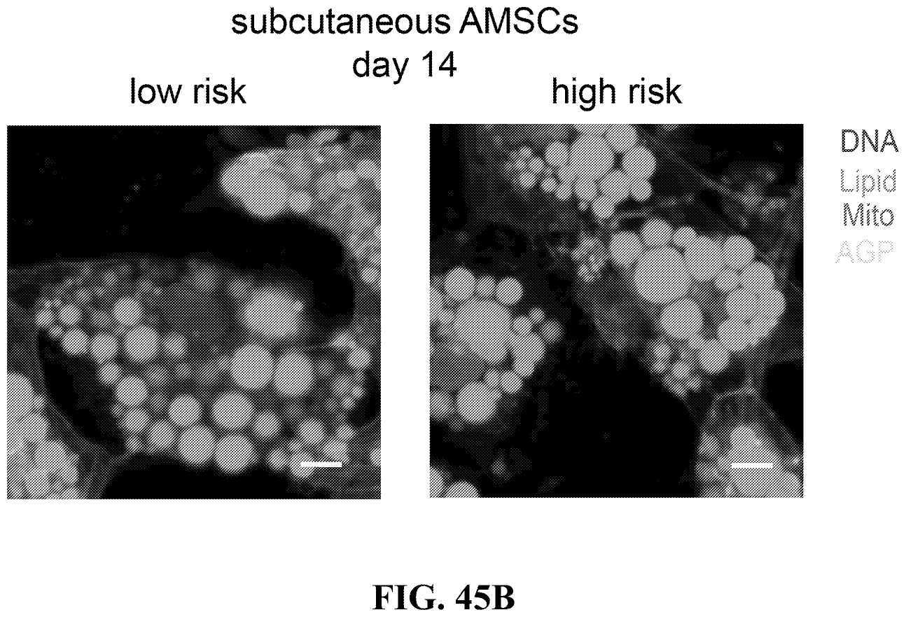

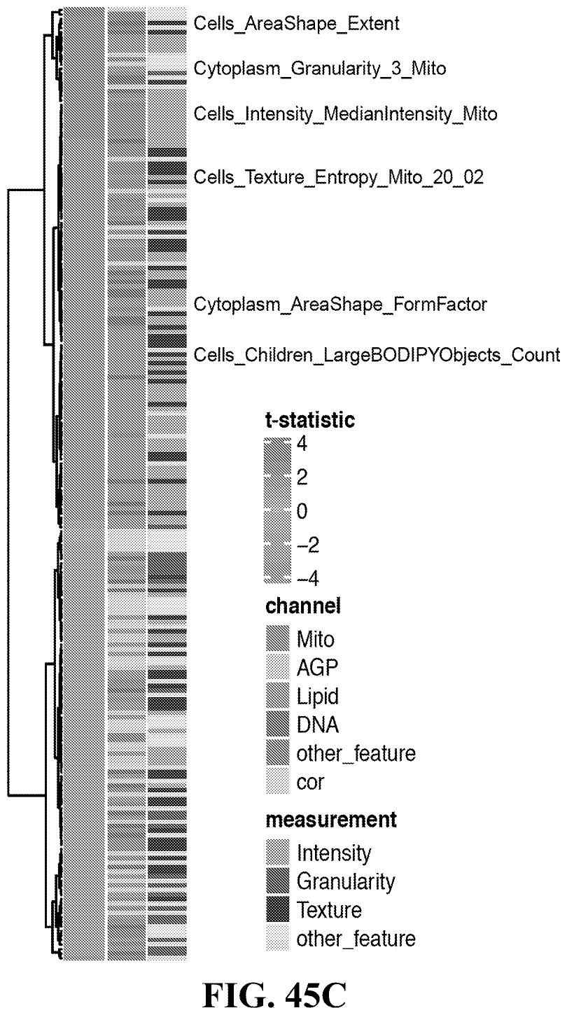

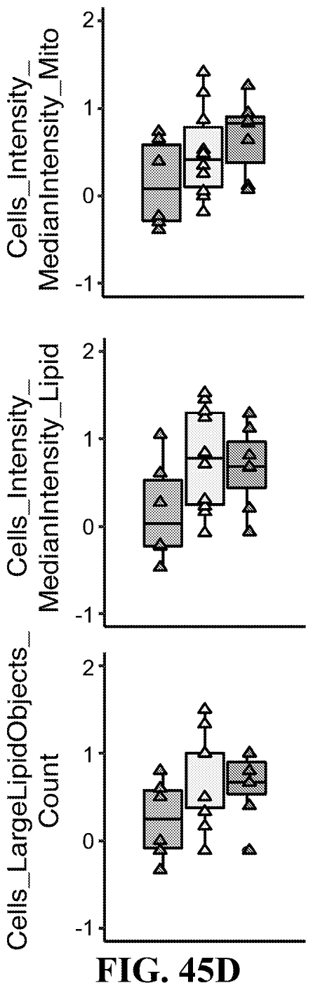

Applicants finally sought to decipher whether the mechanisms identified for rs12454712 would align with global cellular drivers of polygenic risk for increased WHRadjBMI. Applicants compared morphological profiles of high and low polygenic risk female individuals for WHRadjBMI (FIG. 45A) (see Example 1 and 3, and Methods for details) and observed significant morphological differences between subcutaneous adipocytes of low and high risk groups (FIG. 45C). Subcutaneous adipocytes from high risk carriers had higher lipid intensity, higher mitochondrial-related intensity and higher count of Lipid-related objects (FIG. 45D), which was also visible in images of average cells (FIG. 45B). To identify possible mediating pathways, Applicants used a linear regression model of LipocyteProfiler features and transcriptome-wide gene expression data from matched AMSCs at day 14 of differentiation and identified 2429 genes that were connected with at least 5 WHRadjBMI-mediated features. More specifically, the identified WHRadjBMI morphological profile was enriched for genes involved in deficiency of tricarboxylic acid cycle (TCA) pathway (WP2453; WP78), fatty acid oxidation (WP143, WP368), and apoptosis modulation and signaling (WP1772) (Table 22), similar to what Applicants observed for the rs12454712 haplotype in subcutaneous AMSCs (see FIG. 43). Applicants' data suggest that polygenic risk of adverse body fat distribution converges on apoptotic pathways characterised by mitochondrial impairment and that apoptosis is a central/mediating pathway in common genetic risk of adverse body fat distribution (Loh et al. 2020). Increased susceptibility to apoptosis of subcutaneous adipocytes would result in a depletion of peripheral fat storage capacity which in turn is linked to adverse metabolic effects.

Taken together, Applicants have deciphered multiple mechanisms underlying a metabolic risk locus of previously unknown function that presents pleiotropy at every layer of its regulatory circuitry. Applicants have shown that rs12454712 regulates at least three target genes, in three tissues with distinct cellular and morphological consequences, that converge to modulate disease susceptibility and together manifest in a complex metabolic phenotype. Applicants' findings highlight the complexities that one encounters when dissecting disease-associated loci in humans. Here Applicants have showcased a framework based on integration of high content imaging coupled with transcriptomics in a relatively small set of primary human AMSCs that enables unbiased mechanistic interrogation of genetic risk loci. Specifically, this allowed us to i) unravel the spatio-temporal complexities of a risk locus that modulated target gene expression at a specific developmental window and manifested in cellular phenotypes at another, and to ii) identify cellular mechanism by comparing haplotype-driven morphological profiles with signatures of cellular traits (e.g. ROS, apoptosis and thermogenesis).

In conclusion, natural genetic variation in human primary cells manifested in cellular profiles that made it possible to assign molecular mechanisms of the rs12454712 locus that are consistent with an organismal phenotype of adverse body fat distribution and metabolic disease.

### Methods—BCL2, KDSR, VPS4B

BCL2 Silencing Using siRNA

All silencing experiments were performed on 4 technical replicates. One day before silencing, AMSCs were plated into 96-well plates with 10K cells/well using growth medium. RNA-based silencing of BCL2 was performed using RNAiMAX Reagent (ThermoFisher #13778075) and following the manufacturer's protocol. Briefly, Lipofectamine® RNAiMAX Reagent was diluted in Opti-MEM medium (Gibco, Cat #11058021). At the same time, siRNA was diluted in Opti-MEM medium. Then, diluted siRNA was added to the diluted Lipofectamine® RNAiMAX reagent at a ratio 1:1 and incubated for 5 min. The concentration of reagents per well in a 96-well plate were 0.5 μl (10 μM) of silencing oligo (Ambion Cat #4392421, ID s1915) or negative control duplex (Ambion Cat #4390844), and 1.5 μl of lipofectamine RNAiMAX Reagent. The plate was gently swirled and placed in a 37° C.′ incubator at 5% CO2 for three days. Cells were then induced to differentiate following the standard differentiation cocktail or harvested for gene expression analysis to assess knockdown efficiency.

RNA Preparation and qPCR

Total RNA was extracted with Trizol (Ambion 15596026) and the Direct-zol RNA MiniPrep Kit (Zymo R2052) following the manufacturer's instructions. cDNA was synthesized with High-Capacity cDNA Reverse Transcription Kit (Applied Biosystems 4368814) following the manufacturer's instructions. qPCR was performed using Thermo Scientific PCR Master Mix (Thermo Scientific K0172) and taqman probes for target gene BCL2 (Thermo Scientific, Cat #4448892, II Hs04986394_s1) and housekeeping gene CANX (Thermo Scientific, Cat #4448892, ID Hs01558409_m1). Relative gene expression was calculated by the delta delta Ct method. Target gene expression was normalized to expression of CANX.

### Abdominal Laparoscopy Cohort—Munich Obesity BioBank/MOBB

### Samples

Applicants obtained subcutaneous and visceral adipose tissue histology slides from a total of 188 morbidly obese male (35%) and female (65%) patients undergoing a range of abdominal laparoscopic surgeries (sleeve gastrectomy, fundoplication or appendectomy). The visceral adipose tissue is derived from the proximity of the angle of His and subcutaneous adipose tissue obtained from beneath the skin at the site of surgical incision. Images were acquired at 20× magnification with a micron per pixel value of 0.193 μm/pixel. Collagenase digestion and size determination of mature adipocytes was performed as described previously. All samples had genotypes called using the Illumina Global Screening beadchip array.

### Quality Control of Genotyping Data

### Sample Quality Control

DNA was extracted and sent to the Oxford Genotyping Center for genotyping on the Infinium HTS assay on Global Screening Array bead-chips. Genotype QC was done using GenomeStudio and genotypes were converted into PLINK format for downstream analysis.

Applicants checked sample missingness but found no sample with missingness >5%. For the remaining sample quality control (QC) steps, Applicants reduced the genotyping data down to a set of high-quality SNPs. These SNPs were:
(a) Common (minor allele frequency >10%)
(b) Had missingness <0.1%
(c) Independent, pruned at a linkage disequilibrium (r2) threshold of 0.2
(d) Autosomal only
(e) Outside the lactase locus (chr2), the major histocompatibility complex (MHC, chr6), and outside the inversions on chr8 and chr17.
(f) InHardy-Weinberg equilibrium (P>1×10⁻³)

Using the remaining ˜65,000 SNPs, Applicants checked samples for inbreeding (--het in PLINK), but found no samples with excess homozygosity or heterozygosity (no sample >6 standard deviations from the mean). Applicants also checked for relatedness (--genome in PLINK) and found one pair of samples to be identical; Applicants kept the sample with the higher overall genotyping rate. Finally, Applicants performed PCA using EIGENSTRAT and projected the samples onto data from HapMap3, which includes samples from 11 global populations. Six samples appeared to have some amount of non-European ancestral background, while the majority of samples appeared to be of European descent. Applicants removed no samples at this step, selecting to adjust for principal components in genome-wide testing. However, adjustment for principal components failed to eliminate population stratification, and Applicants therefore restricted to samples of European descent only, defined as samples falling within +/−10 standard deviations of the first and second principal component values of the CEU (Northern and Western European-ancestry samples living in Utah) and TSI (Tuscans in Italy) samples included in the HapMap 3 dataset.4 2.43. Finally, sex information was received after initial sample QC was complete. As a result, one sample with potentially mismatching sex information (comparing genotypes and phenotype information) was discovered after analyses were complete and therefore remained in the analysis.

### SNP Quality Control

Applicants removed all SNPs with missingness >5% and out of HWE, P<1×10⁻⁶. Applicants also removed monomorphic SNPs. Finally, Applicants set heterozygous haploid sites to missing to enable downstream imputation.

The final cleaned dataset included 190 samples and ˜700,000 SNPs. Applicants note that histology data was not available for all genotyped samples.

### Genotype Imputation

For the genotyped cohorts without imputation data (ENDOX and MOBB) Applicants performed imputation via the Michigan Imputation Server. Applicants aligned SNPs to the positive strand, and then uploaded the data (in VCF format) to the server. Applicants imputed the data with the Haplotype Reference Consortium (HRC) panel, to be consistent with the fatDIVA data which was already imputed with the HRC panel. Applicants selected EAGLE as the phasing tool to phase the data. To impute chromosome X, Applicants followed the server protocol for imputing this chromosome (including using SHAPEIT to perform the phasing step).

### Human Primary AMSCs Isolation and Differentiation

Human liposuction material used for isolation of preadipocytes was obtained from a collaborating private plastic surgery clinic Medaesthetic Privatklinik Hoffmann & Hoffmann in Munich, Germany. Harvested subcutaneous liposuction material was filled into sterile 1 L laboratory bottles and immediately transported to the laboratory in a secure transportation box. The fat was aliquoted into sterile straight-sided wide-mouth jars, excluding the transfer of liposuction fluid. The fat was stored in cold Adipocyte Basal medium (AC-BM) at a 1:1 ratio of fat to medium and stored at 4° C. to be processed the following day. Additionally, small quantities of the original liposuction material would be aliquoted into T-25 flasks at a 1:1 ratio of fat to medium as controls to check for contamination. These control flasks were stored in the 37° C.′ incubator and were not processed. Krebs-Ringer Phosphate (KRP) buffer was prepared containing 200 U/ml of collagenase and 4% heat shock fraction BSA and sterilized by filtration using a BottleTop Filter 0.22 μm. When the fat reached RT, 12.5 ml of liposuction material was aliquoted into sterile 50-ml tubes with plug seal caps. The tubes were filled to 47.5 ml with warm KRP-BSA-collagenase buffer and the caps were securely tightened and wrapped in Parafilm to avoid leakage. The tubes were incubated in a shaking water bath for 30 minutes at 37° C. with strong shaking. After 30 minutes, the oil on top was discarded and the supernatant was initially filtered through a 2000-μm nylon mesh. The supernatant of all tubes was combined after filtration and centrifuged at 200×g for 10 minutes. The supernatant was discarded and each pellet was resuspended with 3 ml of erythrocyte lysis buffer, then all the pellets were pulled in one tube and incubated for 10 minutes at RT. The cell suspension was filtered through a 250 μm Filter and then through 150 μm Filter, followed by centrifugation at 200 g for 10 minutes. The supernatant was discarded and the pellet containing preadipocytes was resuspended in an appropriate amount of DMEM/F12 with 1% P/S and 10% FCS and seeded in T75 cell culture flasks and stored in the incubator (37° C.′, 5% CO2). The next day the medium was changed to PAC-PM. Once preadipocytes reached 100% confluency in T25 or T75 flasks they were split into 6-well plates at a seeding density on 250,000 cells per plate in PAC-PM. Once they reached 100% confluency, PAC-IM was prepared fresh and added to the preadipocytes to induce differentiation. On day 3 after induction, the medium was changed to PAC-DM and replaced twice a week.

### Iso Tissue

Subcutaneous adipose tissue was sampled from the abdominal area at the site of incision and visceral adipose tissue from the angle of his from patients undergoing elective abdominal laparoscopic surgery. Each patient gave written informed consent prior to inclusion and the study protocol was approved by the ethics committee of the Technical University of Munich (Study nr. 5716/13). Connective tissue and blood vessels were dissected and one gram of minced adipose tissue was digested with 5 ml of Krebs-ringer phosphate buffer containing 200 U/ml of collagenase (SERVA, Heidelberg, Germany). Digestion was carried out at 37° C. for 60 minutes in a shaking water bath. Afterwards the suspension was centrifuged at 200 g for 10 minutes and the supernatant was discarded. The pellet containing the SVF was resuspended in DMEM/F12 (Gibco, Thermo Fisher Scientific, Darmstadt) containing 10% FCS (F7524, Sigma-Aldrich, Taufkirchen, Germany) and 1% penicillin-streptomycin (P/S; PAA Laboratories, Linz, Austria). After filtering the cell suspension through a 70 μm cell strainer the cells were plated, washed with PBS on the next day and medium was changed to proliferation medium. Proliferation and differentiation of isolated preadipocytes was carried out as described earlier. [DOI: 10.1056/NEJMoa1502214]

### Iso Liposuction

Human primary AMSCs were isolated from liposuction material. Each patient gave written informed consent prior to inclusion and the study protocol was approved by the ethics committee of the Technical University of Munich (study nr. 5716/13). The liposuction material was immediately transported to the laboratory and stored with an equal amount of DMEM-F12 (Gibco, Thermo Fisher Scientific, Darmstadt) containing 1% penicillin-streptomycin (P/S; PAA Laboratories, Linz, Austria) over night at 4° C. On the next day the samples were digested in a 1:4 ration with Krebs-Ringer Phosphate (KRP) buffer containing 200 U/ml collagenase (SERVA, Heidelberg, Germany) at 37° C. in a shaking water bath for 60 minutes. After digestion the adipocyte/oil containing layer was removed and the remaining liquid containing the SVF was filtered through a 2000 μm nylon mesh. The SVF was pelleted through centrifugation for 10 minutes at 200 g. The supernatant was discarded and the pellet was resuspended in 37° C.′ warm erythrocyte lysis buffer (155 mM NH₄Cl, 5.7 mM K2HPO₄, 0.1 mM EDTA dihydrate) and incubated at room temperature for 10 minutes. The cell suspension was filtered through a 250 μm Filter and then through a 150 μm Filter, followed by centrifugation at 200 g for 10 minutes. The supernatant was discarded and the pellet containing AMSCs was resuspended in DMEM/F12 containing 1% P/S and 10% FCS (Sigma, F7524). Cells were seeded and washed with PBS on the next day before switching to proliferation medium. Proliferation and differentiation was carried out as described earlier. [DOI: 10.1056/NEJMoa1502214]

### Flow Cytometry

Purity of AMCSs was assessed as previously described (Raajendiran et al, 2019). Briefly, cells were stained with 0.05 ug CD34, 0.125 ug CD29, 0.375 ug CD31, 0.125 ug CD45 per 250K cells and analyzed on CytoFlex together with negative control samples of corresponding AMCSs. (FIG. 46A-46E)

### Differentiation of Human AMSCs

For imaging, cells were seeded at 10K cells/well in 96-well plates (Cell Carrier, Perkin Elmer #6005550) and induced 4 days after seeding. For RNAseq, cells were seeded at 40K cells/well in 12-well dishes (Corning). Before Induction cells were cultured in proliferation medium. Adipogenic differentiation was induced by changing culture medium to induction medium. On day 3 of adipogenic differentiation culture medium was changed to differentiation medium. Medium was changed every 3 days. Visceral-derived AMSCs were differentiated by adding FFA.

### Lipocyte Painting in Human AMSCs

Human primary AMSCs were plated and differentiated in 96-well CellCarrier plates (Perkinelmer/6005550) for 14 days for high content imaging at day 0, day 3, day 8 and day 14 of adipogenic differentiation. On the respective day of the assay, cell culture media was removed and replaced by 0.5 uM Mitotracker staining solution (1 mM MitoTracker Deep Red stock (Invitrogen #M22426) diluted in culture media) to each well followed by 30 minutes incubation at 37° C. protected from light. After 30 min Mitotracker staining solution was removed and cells were washed twice with Dulbecco's Phosphate-Buffered Saline (1×), DPBS (Corning® #21-030-CV) and 2.9 uM BODIPY staining solution (3.8 mM BODIPY 505/515 stock (Thermofisher #D3921) diluted in DPBS) was added followed by 15 minutes incubation at 37° C.′ protected from light. Subsequently, cells were fixed by adding 16% Methanol-free Paraformaldehyde, PFA (Electron Microscopy Sciences #15710-S) directly to the BODIPY staining solution to a final concentration of 3.2% and incubated for 20 minutes at RT protected from light. PFA was removed and cells were washed once with Hank's Balanced Salt Solution (1×), HBSS (Gibco #14025076). To permeabilize cells 0.1% Triton X-100 (Sigma Aldrich #X100) was added and incubated at RT for 10 minutes protected from light. After Permeabilization multi-stain solution (10 units of Alexa Fluor™ 568 Phalloidin (ThermoFisher #A12380), 0.01 mg/ml Hoechst 33342 (Invitrogen #H3570), 0.0015 mg/ml Wheat Germ Agglutinin, Alexa Fluor™ 555 Conjugate (ThermoFisher #/W32464), 3 uM SYTO™ 14 Green Fluorescent Nucleic Acid Stain (Invitrogen #/S7576) diluted in HBSS) was added and cells were incubated at RT for 10 minutes protected from light. Finally, staining solution was removed and cells were washed three times with HBSS. Cells were imaged using a Opera Phenix High content screening system. Per well we imaged 25 fields.

### LipocyteProfiler

Quantitation was performed using CellProfiler 3.1.9. Prior to processing, flat field illumination correction was performed using functions generated from the mean intensity across each plate. Nuclei were identified using the DAPI stain and then expanded to identify whole cells using the AGP and Bodipy stains. Regions of cytoplasm were then determined by removing the Nuclei from the Cell segmentations. Speckles of Bodipy staining were enhanced to assist in detection of small and large individual Bodipy objects. For each object set measurements were collected representing size, shape, intensity, granularity, texture, colocalisation and distance to neighbouring objects.

After feature extraction data was filtered by applying automated and manual quality control steps. First, fields with a total cell count less than 50 cells were removed. Second, fields that are corrupted by experimental induced technical artifacts were removed by applying a manually defined quality control mask. Furthermore, blocklisted features that are known to be noisy and generally unreliable were removed. After filtering data were normalised per plate using a robust scaling approach that subtracts the median from each variable and divides it by the interquartile range. For each individual wells were aggregated for downstream analysis by cell depot and day of differentiation.

Subsequent data analyses were performed in R3.6.1 and Matlab using base packages unless noted. To check for batch effects, Applicants visualised the data using a Principle component analysis and quantifying using a Kolmogorov-Smirnov test implemented in the “BEclear” R package. Additionally, Applicants performed a k-nearest neighbour (knn) supervised machine learning algorithm implemented in the “class” R package (Venables W N, Ripley B D (2002)) to accuracy of predicting. For that the data set, consistent of 3 different cell Types (hWAT, hBAT, SGBS) balanced distributed on the 96-well plate, imaged at 4 days of differentiation, was splitted into equally distributed testing (n=18) and training (n=56) sets. Accuracy of the classification model was predicted based on three different categories cell type, batch and column of the 96-well plate.

For dimensionality reduction visualization Uniform manifold approximation and projection maps (UMAP) were created using the UMAP R package (McInnes and Healy 2018).

To identify patterns of adipocyte differentiation underlying the morphological profiles a sample progression discovery analysis (SPD) was performed using the algorithm described by Peng Qiu et al. Briefly, the two adipose depots were analysed separately and features were clustered into modules based on correlation (correlation coefficient 0.6). Minimal spanning trees (MST) were constructed for each module and MSTs of each module are correlated to each other. Modules that support common MST were selected and an overall MST based on features of all selected modules is reconstructed.

Variance component analysis was performed by fitting multivariable linear regression models:

$$yi \sim xi + zi + …$$

where y denotes an LipocyteProfiler feature of individual i and x, z, etc. independent variables that could confound the variability of the dataset. Independent variables are day of differentiation, experimental batch, column in 96-well plate, adipose depot, free fatty acid treatment, passaging before freezing, season and year of AMSCs isolation, sex, age, T2D status of individual, LipocyteProfiler feature Cells_Neighbors_PercentTouching_Adjacent corresponding to density of cell seeding and identification numbers of individuals.

To test whether there is a difference of morphological profiles in tail ends of polygenic risk scores (PRS) a multi-way analysis of variance (ANOVA) was performed. For that, individuals belonging to top 25% and bottom 25% of PRS score distribution are categorized into a categorical variable with 2 levels, top 25% or 25% bottom, according to their PRS percentile. Differences in morphological profiles are predicted using the categorized PRS variable adjusted for sex, age, BMI and batch.

To overcome multiple testing burden p-values were corrected using false positive rate (FDR) described in R package “qvalue” (Storey J D, Bass A J, Dabney A, Robinson D, 2020). Features with FDR <5% were classified to be significantly impacted by PRS variable. RNA Silencing

Pre-adipocytes were seeded to be 60-70% confluent at time of transfection. Silencing was performed using Lipofectamine® RNAiMAX Transfection Reagent (ThermoFisher #13778075) and following the manufacturer's protocol. Briefly, Lipofectamine® RNAiMAX Reagent was diluted in Opti-MEM medium. At the same time, siRNA was diluted in Opti-MEM medium. Then, diluted siRNA was added to the diluted Lipofectamine®) RNAiMAX reagent at a ratio 1:1 and incubated for 5 min. All silencing experiments were performed on 4 technical replicates. The plate was gently swirled and placed in a 37° C. incubator at 5% CO2 for 48 hours. Cells were then induced to differentiate following the standard differentiation cocktail or harvested for gene expression analysis and to assess knockdown efficiency. Silencing efficiency was compared between experiments using RT-qPCR with taqman probes for BCL2 (Assay Id U.S. Ser. No. 04/986,394 s1) and CANX as a housekeeping control gene (see RT qPCR section for detailed methods).

Silencer Select Pre-designed siRNA for BCL2 (ambion life technologies, #4392421, s1915) and Silencer Select Negative Control (ambion life technologies, #4390844), were diluted to 100 uM in water.

### Seahorse

The protocol for a standard bioenergetics profile is composed of basal mitochondrial respiration, ATP turnover, proton leak and mitochondrial respiratory capacity. First, oxygen consumption rate (OCR) in basal conditions was determined and used to calculate the basal mitochondrial respiration. After this, 2 μM oligomycin was injected from the first port to inhibit ATP synthase, resulting in an accumulation of protons in the mitochondrial intermembrane space and a reduced activity of the electron transport chain. The resulting decrease in OCR reveals the respiration driving ATP synthesis in the cells, indicating ATP turnover. Residual oxygen consumption capacity can be attributed to the proton leak maintaining a minimal ETC and non-mitochondrial respiration. Next, 2 μM of the mitochondrial uncoupler FCCP was injected which results in an increase in OCR as the proton gradient across the inner mitochondrial membrane is dissipated and ETC resumed. This measurement reflects the maximal mitochondrial respiratory capacity. Finally, 2 μM Rotenone/Antimycin A are injected to completely stop ETC activity and the OCR reading at this phase reflects non-mitochondrial respiration. We normalized all data to the relative number of live cells in each well of the 96-well Seahorse plate.

Oxygen Consumption and Bioenergetics Profile was measured using the XF24 extracellular flux analyzer from Seahorse Bioscience. The protocol used in this assay was adapted from Gesta et al., 2011. For this assay, pre-adipocytes were counted and 10K cells per well were seeded onto seahorse 96 well plate in 50 μl of growth media and left to adhere overnight. The next day, silencing was performed as seen in the previous section. Three days later, cells were induced to differentiate within the seahorse plate following the adipogenic differentiation protocol as described previously. Each cell type was run in 8 replicates. When the cells were terminally differentiated at day 14 post adipogenic induction, the assay was performed. The evening before the assay, the seahorse XF-24 instrument cartridge was loaded with seahorse calibrant and placed in a CO2-free incubator at 37° C. overnight.

On the day of the assay, cells were washed in XF Assay Media, L-glutamine 2 mM, sodium pyruvate 2 mM, and glucose 10 mM (pH was measured and adjusted to pH7.4 at 37° C.). The seahorse plate containing the differentiated adipocytes was then incubated for at least 1 hour at 37° C. in a CO2-free incubator to allow CO2 to diffuse out of solution. According to the manufactures protocol, the ports of the seahorse XF-24 analyser cartridge were then loaded with the following compounds:
Port A: Oligomycin (complex 1 inhibitor)
Port B: FCCP (carbonyl cyanide-p-trifluoromethoxyphenylhydrazone; mitochondrial uncoupler)
Port C: Rotenone and Antimycin (inhibitors of electron transfer)

Before running the assay, the XF-24 instrument cartridge was calibrated.

For total oxygen consumption rate (OCR) measurements, the minimum OCR reading after Rotenone/Antimycin A treatment was subtracted from the initial untreated level, following the manufacturer's protocol. To directly measure mitochondrial thermogenesis, uncoupled respiration (proton leak) was measured by subtracting the minimum OCR level after Rotenone/Antimycin from the minimum level after oligomycin treatment. Oxygen concentrations were measured over time periods of 4 min with 2 min waiting and 2 min mixing.

### TABLES

TABLE 1
Lists of pathways enriched among representative significant gene LP-features
connections. Term, which pathway; Overlap, number of genes that overlap and
total genes; p-value, enrichment p-value; adj., adjusted p-value, q-value;
OR, odds ratio, enrichment; CS, combined score, approximation of overall
association (-log10(P) * log(Odds)), Genes, genes in the pathway which
are associated with gene LP-feature connections.

| | | | | | | |
|---|---|---|---|---|---|---|
| Term | Overlap | p-value | adj. | OR | CS | Genes |

BioPlanet 2019: Cells_Granularity_11_Mito

| | | | | | | |
|---|---|---|---|---|---|---|
| Tricarboxylic acid (TCA) | 6/39 | 4.2E−06 | 1.0E−03 | 16.62 | 205.77 | CS; PDP2; SUCLG2; |
| cycle | | | | | | SDHC; DLAT; PDK2 |
| Pyruvate metabolism and | 6/40 | 4.9E−06 | 1.0E−03 | 16.13 | 197.23 | CS; PDP2; SUCLG2; |
| citric acid (TCA) cycle | | | | | | SDHC; DLAT; PDK2 |
| Tricarboxylic acid | 9/117 | 6.4E−06 | 1.0E−03 | 7.70 | 92.08 | CS; NDUFA6; PDP2; |
| (TCA) cycle and | | | | | | NDUFA5; NDUFB5; |
| respiratory electron | | | | | | SUCLG2; SDHC; |
| transport | | | | | | DLAT; PDK2 |
| Pyruvate dehydrogenase | 3/12 | 2.8E−04 | 3.3E−02 | 30.09 | 246.63 | PDP2; DLAT; PDK2 |
| (PDH) complex regulation | | | | | | |
| Vibrio cholerae | 5/55 | 3.6E−04 | 3.4E−02 | 9.09 | 72.17 | TJP1; ATP6V1E1; |
| infection | | | | | | PRKACA; PDIA4; |
| | | | | | | TJP2 |
| Metabolism | 33/1615 | 4.7E−04 | 3.7E−02 | 2.01 | 15.38 | DMGDH; NDUFB5; |
| | | | | | | HSD17B4; CACNA1C; |
| | | | | | | HSD17B8; GLIPR1; |
| | | | | | | DBT; DLAT; ATP6V |
| | | | | | | 1E1; PDK2; NDUFA6; |
| | | | | | | NDUFA5; ACSL1; |
| | | | | | | MMAB; NCOA3; NFYC; |
| | | | | | | CAD; TALDO1; G0S2; |
| | | | | | | SDHC; FAH; PGD; |
| | | | | | | DHRS3; CS; GLUD1; |
| | | | | | | UGDH; MLXIPL; PDP2; |
| | | | | | | G6PC3; POLR3C; |
| | | | | | | ACER3; SUCLG2; |
| | | | | | | CSPG5 |
| PKA-mediated phosphory- | 2/5 | 1.2E−03 | 8.1E−02 | 59.92 | 403.03 | MLXIPL; PRKACA |
| lation of key metabolic | | | | | | |
| factors | | | | | | |

WikiPathway: Cells_Intensity_MedianIntensity_Lipid

| Electron Transport Chain | 20/103 | 2.2E−13 | 6.58E−11 | 10.61 | 309.15 | NDUFA9; NDUFA8; |
|---|---|---|---|---|---|---|
| (OXPHOS system in | | | | | | ATP6; SDHC; SDHD; |
| mitochondria) WP111 | | | | | | COX6A1; SDHA; |
| | | | | | | COX5A; ATP5F1B; |
| | | | | | | SCO1; COX3; COX2; |
| | | | | | | COX11; UQCRFS1; |
| | | | | | | COX1; UQCRC2; CYTB; |
| | | | | | | ND2; SLC25A4; ND4 |
| Mitochondrial LC-Fatty | 8/17 | 1.6E−09 | 2.32E−07 | 38.24 | 775.72 | HADHA; ACADVL; CPT2; |
| Acid Beta-Oxidation | | | | | | EHHADH; SLC25A20; |
| WP368 | | | | | | HADH; ACADS; ACSF2 |
| Fatty Acid Beta | 10/34 | 3.1E−09 | 3.13E−07 | 17.99 | 352.24 | GCDH; LIPE; HADHA; |
| Oxidation WP143 | | | | | | ACADVL; CPT2; LPL; |
| | | | | | | SLC25A20; HADH; |
| | | | | | | ACADS; DLD |
| Nonalcoholic fatty liver | 18/155 | 2.1E−08 | 1.54E−06 | 5.74 | 101.63 | NDUFA9; CEBPA; |
| disease WP4396 | | | | | | NDUFA8; ADIPOQ; |
| | | | | | | SDHC; SDHD; COX6A1; |
| | | | | | | SDHA; COX5A; COX3; |
| | | | | | | AKT1; COX2; CYCS; |
| | | | | | | UQCRFS1; COX1; |
| | | | | | | UQCRC2; CYC1; CYTB |
| TCA Cycle (aka Krebs or | 7/18 | 8.6E−08 | 5.14E−06 | 27.31 | 444.43 | CS; SDHC; SDHD; |
| citric acid cycle) WP78 | | | | | | ACO2; SDHA; DLD; |
| | | | | | | IDH3A |
| TCA Cycle and Deficiency | 6/16 | 9.7E−07 | 4.84E−05 | 25.69 | 355.83 | CS; PDHA1; ACO1; |
| of Pyruvate Dehydro- | | | | | | SDHA; DLD; IDH3A |
| genase | | | | | | |
| complex (PDHc) WP2453 | | | | | | |
| PPAR signaling pathway | 10/67 | 3.0E−06 | 1.3E−04 | 7.56 | 96.16 | SLC27A1; FABP4; CPT2; |
| WP3942 | | | | | | EHHADH; ADIPOQ; AQP7; |
| | | | | | | LPL; PLIN1; ACAA1; |
| | | | | | | RXRG |
| Lipid Metabolism Pathway | 7/29 | 3.4E−06 | 1.3E−04 | 13.65 | 171.97 | LIPE; PDHA1; PRKAR2B; |
| WP3965 | | | | | | AKT1; ABHD5; PLIN1; |
| | | | | | | PRKACA |
| Amino Acid metabolism | 11/91 | 8.0E−06 | 2.7E−04 | 5.93 | 69.65 | CS; GLUD1; PDHA1; |
| WP3925 | | | | | | MARS2; EHHADH; PDK4; |
| | | | | | | ACO2; HADH; ACAA1; |
| | | | | | | SDHA; DLD |
| Tryptophan metabolism | 7/46 | 8.2E−05 | 2.5E−03 | 7.69 | 72.34 | ALDH3A2; GCDH; ALDH2; |
| WP465 | | | | | | AFMID; CAT; HADH; |
| | | | | | | ALDH9A1 |
| Cerebral Organic | 3/7 | 4.0E−04 | 1.1E−02 | 31.92 | 249.73 | GCDH; ADHFE1; L2HGDH |
| Acidurias, including | | | | | | |
| diseases WP4519 | | | | | | |
| Nanomaterial induced | 4/20 | 1.0E−03 | 2.4E−02 | 10.66 | 73.45 | ENDOG; AIFM1; BCL2; |
| apoptosis WP2507 | | | | | | CYCS |
| Sterol Regulatory | 7/69 | 1.1E−03 | 2.4E−02 | 4.83 | 33.13 | CDK8; AKT1; LPL; |
| Element-Binding | | | | | | PRKACA; SEC24D; |
| Proteins (SREBP) | | | | | | PPARGC1B; MTOR |
| signalling WP1982 | | | | | | |
| Fatty Acid | 4/22 | 1.5E−03 | 3.0E−02 | 9.47 | 61.73 | MECR; ACAA2; ECH1; |
| Biosynthesis WP357 | | | | | | HADH |
| PI3K/AKT/mTOR - VitD3 | 4/22 | 1.5E−03 | 3.0E−02 | 9.47 | 61.73 | PDHA1; VDR; AKT1; |
| Signalling WP4141 | | | | | | |
| Oxidative phosphory- | 6/60 | 2.6E−03 | 4.4E−02 | 4.75 | 28.32 | NDUFA9; NDUFA8; |
| lation WP623 | | | | | | ATP5F1B; ATP6; |
| | | | | | | ND2; ND4 |
| Metabolic reprogram- | 5/42 | 2.7E−03 | 4.4E−02 | 5.77 | 34.09 | GLUD1; PDHA1; |
| ming in colon cancer | | | | | | PYCR2; ACO2; |
| WP4290 | | | | | | IDH3A |
| PPAR Alpha Pathway | 4/26 | 2.8E−03 | 4.4E−02 | 7.75 | 45.52 | SLC27A1; CPT2; |
| WP2878 | | | | | | EHHADH; ACAA1 |
| Valproic acid | 3/13 | 2.9E−03 | 4.4E−02 | 12.76 | 7 4.37 | HADHA; EHHADH; |
| pathway WP3871 | | | | | | ACADSB |
| Nuclear Receptors | 16/319 | 3.2E−03 | 4.4E−02 | 2.28 | 13.07 | SLC27A1; ALAS1; |
| Meta-Pathway | | | | | | VDR; ARNT; |
| WP2882 | | | | | | SLC39A14; POU5F1; |
| | | | | | | SLC2A8; DNAJB1; |
| | | | | | | GCLC; CPT2; |
| | | | | | | EHHADH; PDK4; |
| | | | | | | SPRY1; ACAA1; |
| | | | | | | CES2; EPHA2 |
| Follicle Stimulating | 4/27 | 3.2E−03 | 4.4E−02 | 7.41 | 42.49 | AKT1; PRKACA; |
| Hormone (FSH) | | | | | | RAF1; MTOR |
| signaling pathway | | | | | | |
| WP2035 | | | | | | |
| Adipogenesis WP236 | 9/130 | 3.3E−03 | 4.4E−02 | 3.19 | 18.26 | MEF2A; CNTFR; |
| | | | | | | LIPE; CEBPA; |
| | | | | | | ADIPOQ; LPL; |
| | | | | | | PLIN1; KLF15; |
| | | | | | | RXRG |
| Thermogenesis | 8/108 | 3.6E−03 | 4.7E−02 | 3.43 | 19.28 | LIPE; CPT2; |
| WP4321 | | | | | | PLIN1; SLC25A20; |
| | | | | | | PRKACA; SOS2; |
| | | | | | | ADCY6; MTOR |
| | | | adj. p- | | | |
| Term | Overlap | p-value | value | OR | CS | Genes |

WikiPathway: Cells_Mean_LargeLipidObjects_Granularity_4_Lipid

| Adipogenesis WP236 | 6/130 | 7.2E−04 | 4.9E−02 | 6.04 | 43.72 | MEF2A; CEBPA; |
|---|---|---|---|---|---|---|
| | | | | | | SMAD3; ADIPOQ; |
| | | | | | | FAS; KLF15 |
| Apoptosis Modula- | 5/91 | 9.2E−04 | 4.9E−02 | 7.22 | 50.50 | AIFM2; CASP4; |
| tion and Signaling | | | | | | TNFSF10; BCL2; |
| WP1772 | | | | | | FAS |
| Differentiation of | 3/25 | 1.1E−03 | 4.9E−02 | 16.78 | 114.47 | CEBPA; ADIPOQ; |
| white and brown | | | | | | PPARGC1B |
| adipocyte WP2895 | | | | | | |
| Term | Overlap | p-value | adj. | OR | CS | Genes |

WikiPathway: Cells_Mean_LargeLipidObjects_Correlation_Overlap_Mito_AGP

| | | | | | | |
|---|---|---|---|---|---|---|
| Cytoplasmic | 41/89 | 3.0E−27 | 1.12E−24 | 14.30 | 873.59 | RPL4; RPL5; |
| Ribosomal | | | | | | RPL30; RPL3; |
| Proteins WP477 | | | | | | RPL32; RPL10; |
| | | | | | | RPL31; RPL34; |
| | | | | | | RPL12; RPLP0; |
| | | | | | | MRPL19; RPL10A; |
| | | | | | | RPS4X; RPL7A; |
| | | | | | | RPS19; RPS18; |
| | | | | | | RPL36; RPS3; |
| | | | | | | RPL35; RPL37; |
| | | | | | | RPL15; RPS11; |
| | | | | | | RPS27A; RPS12; |
| | | | | | | RPL19; RPL21; |
| | | | | | | RPS7; RPS8; |
| | | | | | | RPS5; RPS6; |
| | | | | | | RPL13A; RPS3A; |
| | | | | | | RPSA; RPS25; |
| | | | | | | RPS27; RPL27A; |
| | | | | | | RPL37A; RPL24; |
| | | | | | | RPS21; RPS24; |
| | | | | | | RPS23 |
| Fatty Acid Beta | 16/34 | 1.3E−11 | 2.35E−09 | 14.58 | 366.04 | GCDH; ACADVL; |
| Oxidation WP143 | | | | | | CHKB; ACSL1; |
| | | | | | | ECI1; LPL; |
| | | | | | | ACSL5; HADHB; |
| | | | | | | LIPE; HADHA; |
| | | | | | | CPT2; SLC25A20; |
| | | | | | | HADH; ACADS; |
| | | | | | | DLD; CRAT |
| Mitochondrial | 11/17 | 2.2E−10 | 2.74E−08 | 29.97 | 666.46 | PECR; HADHA; |
| LC-Fatty Acid | | | | | | ACADVL; CPT2; |
| Beta-Oxida- | | | | | | ACSL1; ECI1; |
| tion WP368 | | | | | | EHHADH; |
| | | | | | | SLC25A20; HADH; |
| | | | | | | ACADS; ACSF2 |
| PPAR signaling | 18/67 | 3.1E−08 | 2.92E−06 | 6.03 | 104.16 | SLC27A1; ACSL1; |
| pathway WP3942 | | | | | | PDPK1; ADIPOQ; |
| | | | | | | AQP7; ACSL5; |
| | | | | | | LPL; NR1H3; |
| | | | | | | FABP4; CPT2; |
| | | | | | | ACOX1; SCD; |
| | | | | | | EHHADH; CD36; |
| | | | | | | PLIN1; ACAA1; |
| | | | | | | PCK1; PLTP |
| Sterol Regulatory | 16/69 | 1.6E−06 | 1.2E−04 | 4.94 | 66.03 | GSK3A; AMFR; |
| Element-Binding | | | | | | CYP51A1; LPL; |
| Proteins (SREBP) | | | | | | LSS; MTOR; |
| signalling WP1982 | | | | | | CDK8; PIK3CA; |
| | | | | | | GPAM; SCD; |
| | | | | | | AKT1; PRKACA; |
| | | | | | | LPIN1; SEC24D; |
| | | | | | | PPARGC1B; SEC31A |
| TCA Cycle and | 7/16 | 1.6E−05 | 9.9E−04 | 12.67 | 139.99 | CS; IDH1; SUCLG2; |
| Deficiency of | | | | | | ACO1; PCK1; DLD; |
| Pyruvate De- | | | | | | IDH3A |
| hydrogenase | | | | | | |
| complex | | | | | | |
| (PDHc) WP2453 | | | | | | |
| Fatty Acid | 8/22 | 2.0E−05 | 1.0E−03 | 9.31 | 100.95 | PECR; ACAA2; |
| Biosynthesis | | | | | | ACSL1; SCD; |
| WP357 | | | | | | ECH1; ACSL5; |
| | | | | | | HADH; ACACB |
| Insulin signal- | 5/8 | 3.2E−05 | 1.3E−03 | 27.11 | 280.68 | TBC1D4; INSR; |
| ling in human | | | | | | AKT2; RPS6; |
| adipocytes | | | | | | MTOR |
| (diabetic | | | | | | |
| condition) | | | | | | |
| WP3635 | | | | | | |
| Insulin signal- | 5/8 | 3.2E−05 | 1.3E−03 | 27.11 | 280.68 | TBC1D4; INSR; |
| ling in human | | | | | | AKT2; RPS6; |
| adipocytes | | | | | | MTOR |
| (normal condi- | | | | | | |
| tion) WP3634 | | | | | | |
| TCA Cycle (aka | 7/18 | 4.0E−05 | 1.5E−03 | 10.36 | 104.99 | CS; SUCLA2; |
| Krebs or citric | | | | | | SUCLG2; SDHD; |
| acid cycle) | | | | | | ACO2;D LD; |
| WP78 | | | | | | IDH3A |
| Adipogenesis | 20/130 | 6.2E−05 | 2.1E−03 | 2.98 | 28.89 | MEF2A; CNTFR; |
| WP236 | | | | | | STAT5B; CEBPA; |
| | | | | | | SMAD3; ADIPOQ; |
| | | | | | | LPL; NR1H3; |
| | | | | | | NR3C1; KLF15; |
| | | | | | | AGPAT2; BSCL2; |
| | | | | | | LIPE; SCD; |
| | | | | | | PNPLA3; FAS; |
| | | | | | | STAT6; PLIN1; |
| | | | | | | PCK1; LPIN1 |
| Nonalcoholic | 22/155 | 9.3E−05 | 2.9E−03 | 2.71 | 25.16 | NDUFA9; CEBPA; |
| fatty liver | | | | | | GSK3A; NDUFB8; |
| disease | | | | | | NDUFA7; INSR; |
| WP4396 | | | | | | ADIPOQ; NR1H3; |
| | | | | | | SDHD; COX6A1; |
| | | | | | | ADIPOR2; MAPK9; |
| | | | | | | PIK3CA; AKT2; |
| | | | | | | AKT1; CYCS; |
| | | | | | | UQCRFS1; FAS; |
| | | | | | | BAX; CYC1; |
| | | | | | | CYTB; NDUFV2 |
| Lipid | 8/29 | 1.8E−04 | 5.3E−03 | 6.21 | 53.42 | LIPE; PRKAR2B; |
| Metabolism | | | | | | AKT2; HILPDA; |
| Pathway | | | | | | AKT1; ABHD5; |
| WP3965 | | | | | | PLIN1; PRKACA |
| Amino Acid | 14/91 | 7.5E−04 | 2.0E−02 | 2.97 | 21.37 | PDHX; PPM1L; |
| metabolism | | | | | | GPT2; IDH1; CS; |
| WP3925 | | | | | | HMGCL; GLUD1; |
| | | | | | | EHHADH; PDK4; |
| | | | | | | ACO2;HADH;A |
| | | | | | | CAA1; PCK1; |
| | | | | | | DLD |
| Glycerophos- | 6/21 | 9.7E−04 | 2.4E−02 | 6.51 | 45.13 | PCYT2; CHKB; |
| pholipid | | | | | | PTPMT1; GPD1; |
| Biosynthetic | | | | | | CEPT1; LPIN1 |
| Pathway | | | | | | |
| WP2533 | | | | | | |
| Cholesterol | 5/15 | 1.2E−03 | 2.8E−02 | 8.13 | 54.59 | NSDHL; CYP51A1; |
| Biosynthesis | | | | | | SC5D; DHCR7; |
| Pathway | | | | | | LSS |
| WP197 | | | | | | |
| Thermogenesis | 15/108 | 1.5E−03 | 3.2E−02 | 2.63 | 17.21 | KLB; ATF2; |
| WP4321 | | | | | | ACSL1; NPR1; |
| | | | | | | RPS6; ACSL5; |
| | | | | | | ADCY6; MTOR; |
| | | | | | | ACTG1; LIPE; |
| | | | | | | CPT2; GRB2; |
| | | | | | | PLIN1; SLC25A20; |
| | | | | | | PRKACA |
| Estrogen | 6/23 | 1.6E−03 | 3.3E−02 | 5.74 | 36.83 | MAPK9; PIK3CA; |
| signaling | | | | | | GPER1; BCL2; |
| pathway | | | | | | AKT1; PRKACA |
| WP712 | | | | | | |
| Angiopoietin | 17/132 | 1.7E−03 | 3.3E−02 | 2.42 | 15.41 | MAP4K2; GSK3A; |
| Like Protein | | | | | | PDPK1; INSR; |
| 8 Regulatory | | | | | | LPL; NR1H3; |
| Pathway | | | | | | MTOR; MAPK9; |
| WP3915 | | | | | | RPS6KA5; PIK3CA; |
| | | | | | | SCD; AKT2; AKT1; |
| | | | | | | MAP3K10; PCK1; |
| | | | | | | MAP3K14; RHOQ |
| Structural | 9/49 | 1.8E−03 | 3.4E−02 | 3.66 | 23.08 | ATF2; MAPK9; |
| Pathway of | | | | | | RPS6KA5; TOLLIP; |
| Interleukin | | | | | | MKNK2; MBP; |
| 1 (IL-1) | | | | | | IRAK4; MAP3K14; |
| WP2637 | | | | | | MYD88 |
| Triacylgly- | 6/24 | 2.1E−03 | 3.7E−02 | 5.42 | 33.49 | LIPE; DGAT1; |
| ceride | | | | | | GPAM; GPD1; |
| Synthesis | | | | | | LPL; AGPAT2 |
| WP325 | | | | | | |
| Apoptosis | 13/91 | 2.3E−03 | 3.9E−02 | 2.72 | 16.55 | TNFRSF1B; AIFM2; |
| Modulation | | | | | | TRAF3; TOLLIP; |
| and | | | | | | CASP4; BCL2; FAS; |
| Signaling | | | | | | BAX; CYCS; PRKD1; |
| WP1772 | | | | | | MAP3K14; MYD88; |
| | | | | | | BCL2L2 |
| Insulin | 19/160 | 2.4E−03 | 4.0E−02 | 2.20 | 13.25 | MAP4K2; GSK3A; |
| Signaling | | | | | | PDPK1; STXBP1; |
| WP481 | | | | | | INSR; PTPRF; |
| | | | | | | MTOR; LIPE; |
| | | | | | | MAPK9; RPS6KA5; |
| | | | | | | PIK3CA; TBC1D4; |
| | | | | | | AKT2; AKT1; |
| | | | | | | MAP3K10; GRB2; |
| | | | | | | MAP3K14; VAMP2; |
| | | | | | | RHOQ |

TABLE 2
Morphological signatures of adipocyte marker genes SCD, PLIN2, LIPE, TIMM22,
INSR and GLUT4 recapitulate their cellular function. LMM output (significance level FDR
5%). Beta, beta of LMM; se, standard error of LMM, p-value of LMM, q-value of LMM

| | | | | |
|---|---|---|---|---|
| Lipocyte Profiler features | beta | se | p-value | q-value |

SCD (ENSG00000099194)

| | | | | |
|---|---|---|---|---|
| Cells_AreaShape_MeanRadius | 6.3E−08 | 2.1E−08 | 2.5E−03 | 2.6E−02 |
| Cells_AreaShape_MedianRadius | 6.8E−08 | 2.3E−08 | 3.1E−03 | 2.9E−02 |
| Cells_AreaShape_Zernike_7_5 | 1.8E−08 | 6.8E−09 | 7.9E−03 | 4.8E−02 |
| Cells_LipidObjects_AreaShape_Area | 2.3E−07 | 4.8E−08 | 3.2E−06 | 8.5E−04 |
| Cells_LipidObjects_Intensity_IntegratedIntensity_Lipid | 2.5E−07 | 6.7E−08 | 1.9E−04 | 1.2E−02 |
| Cells_Children_LipidObjects_Count | 1.5E−07 | 4.2E−08 | 6.0E−04 | 1.9E−02 |
| Cells_Children_LargeLipidObjects_Count | 2.2E−07 | 7.4E−08 | 3.1E−03 | 2.9E−02 |
| Cells_Correlation_Correlation_Mito_AGP | −1.5E−07 | 5.0E−08 | 3.7E−03 | 3.2E−02 |
| Cells_Correlation_Correlation_Mito_Lipid | −1.5E−07 | 5.4E−08 | 6.5E−03 | 4.2E−02 |
| Cells_Correlation_Overlap_Mito_AGP | −1.4E−07 | 3.7E−08 | 1.6E−04 | 1.0E−02 |
| Cells_Granularity_10_AGP | −5.9E−08 | 2.2E−08 | 8.0E−03 | 4.8E−02 |
| Cells_Granularity_10_Mito | −9.4E−08 | 1.7E−08 | 2.2E−08 | 1.9E−05 |
| Cells_Granularity_11_AGP | −4.2E−08 | 8.9E−09 | 2.2E−06 | 6.3E−04 |
| Cells_Granularity_11_Mito | −1.2E−07 | 1.5E−08 | 1.0E−16 | 8.9E−13 |
| Cells_Granularity_12_Mito | −9.4E−08 | 1.9E−08 | 5.2E−07 | 2.2E−04 |
| Cells_Granularity_15_Lipid | 5.0E−08 | 1.8E−08 | 4.4E−03 | 3.5E−02 |
| Cells_Granularity_7_AGP | −8.4E−08 | 2.4E−08 | 3.7E−04 | 1.8E−02 |
| Cells_Granularity_8_AGP | −7.7E−08 | 2.4E−08 | 1.5E−03 | 2.3E−02 |
| Cells_Granularity_9_AGP | −7.2E−08 | 2.5E−08 | 3.5E−03 | 3.2E−02 |
| Cells_Intensity_IntegratedIntensity_Lipid | 2.5E−07 | 6.7E−08 | 1.7E−04 | 1.1E−02 |
| Cells_Intensity_IntegratedIntensityEdge_Lipid | 2.7E−07 | 6.9E−08 | 7.6E−05 | 6.5E−03 |
| Cells_Intensity_IntegratedIntensityEdge_DNA | 1.1E−07 | 4.0E−08 | 7.5E−03 | 4.7E−02 |
| Cells_Intensity_IntegratedIntensityEdge_Mito | 2.3E−07 | 8.0E−08 | 4.1E−03 | 3.4E−02 |
| Cells_Intensity_LowerQuartileIntensity_Lipid | 3.2E−07 | 7.3E−08 | 1.1E−05 | 2.0E−03 |
| Cells_Intensity_MADIntensity_Lipid | 2.9E−07 | 7.1E−08 | 4.5E−05 | 4.7E−03 |
| Cells_Intensity_MeanIntensity_Lipid | 2.1E−07 | 7.2E−08 | 2.9E−03 | 2.9E−02 |
| Cells_Intensity_MeanIntensityEdge_Lipid | 2.5E−07 | 7.0E−08 | 3.8E−04 | 1.8E−02 |
| Cells_Intensity_MeanIntensityEdge_Mito | 2.7E−07 | 9.1E−08 | 3.5E−03 | 3.2E−02 |
| Cells_Intensity_MedianIntensity_Lipid | 3.2E−07 | 7.1E−08 | 5.8E−06 | 1.3E−03 |
| Cells_Intensity_MinIntensity_AGP | −1.4E−07 | 4.6E−08 | 1.9E−03 | 2.4E−02 |
| Cells_Intensity_MinIntensity_Lipid | 2.3E−07 | 6.0E−08 | 9.1E−05 | 7.4E−03 |
| Cells_Intensity_MinIntensityEdge_AGP | −1.8E−07 | 5.3E−08 | 7.0E−04 | 1.9E−02 |
| Cells_Intensity_MinIntensityEdge_Lipid | 2.4E−07 | 6.0E−08 | 8.5E−05 | 7.0E−03 |
| Cells_Intensity_StdIntensity_AGP | −1.2E−07 | 4.3E−08 | 6.7E−03 | 4.3E−02 |
| Cells_Intensity_UpperQuartileIntensity_Lipid | 2.5E−07 | 7.5E−08 | 7.1E−04 | 1.9E−02 |
| Cells_LargeLipidObjects_AreaShape_Area | 2.5E−07 | 5.9E−08 | 2.9E−05 | 3.7E−03 |
| Cells_LargeLipidObjects_Intensity_IntegratedIntensity_Lipid | 2.6E−07 | 7.2E−08 | 3.8E−04 | 1.8E−02 |
| Cells_Mean_LargeLipidObjects_Correlation_K_AGP_Mito | 2.1E−07 | 7.3E−08 | 4.3E−03 | 3.5E−02 |
| Cells_Mean_LargeLipidObjects_Correlation_K_Lipid_Mito | 1.7E−07 | 6.0E−08 | 4.8E−03 | 3.7E−02 |
| Cells_Mean_LargeLipidObjects_Correlation_K_DNA_Mito | 2.1E−07 | 7.8E−08 | 7.7E−03 | 4.7E−02 |
| Cells_Mean_LargeLipidObjects_Correlation_Overlap_DNA_AGP | −9.9E−08 | 3.3E−08 | 2.4E−03 | 2.6E−02 |
| Cells_Mean_LargeLipidObjects_Correlation_Overlap_Mito_AGP | −1.3E−07 | 2.6E−08 | 2.1E−07 | 1.1E−04 |
| Cells_Mean_LargeLipidObjects_Granularity_1_Lipid | 1.7E−07 | 4.6E−08 | 2.9E−04 | 1.5E−02 |
| Cells_Mean_LargeLipidObjects_Granularity_10_Lipid | 1.6E−07 | 4.3E−08 | 1.4E−04 | 9.5E−03 |
| Cells_Mean_LargeLipidObjects_Granularity_11_Lipid | 1.2E−07 | 4.4E−08 | 5.1E−03 | 3.8E−02 |
| Cells_Mean_LargeLipidObjects_Granularity_12_Lipid | 1.0E−07 | 3.9E−08 | 8.0E−03 | 4.8E−02 |
| Cells_Mean_LargeLipidObjects_Granularity_2_Lipid | 1.7E−07 | 4.9E−08 | 7.6E−04 | 1.9E−02 |
| Cells_Mean_LargeLipidObjects_Granularity_3_Lipid | 2.0E−07 | 4.7E−08 | 1.8E−05 | 2.7E−03 |
| Cells_Mean_LargeLipidObjects_Granularity_4_Lipid | 2.2E−07 | 4.5E−08 | 1.3E−06 | 4.4E−04 |
| Cells_Mean_LargeLipidObjects_Granularity_5_Lipid | 2.3E−07 | 4.8E−08 | 9.7E−07 | 3.5E−04 |
| Cells_Mean_LargeLipidObjects_Granularity_6_Lipid | 2.4E−07 | 4.9E−08 | 1.2E−06 | 4.1E−04 |
| Cells_Mean_LargeLipidObjects_Granularity_7_Lipid | 2.3E−07 | 4.9E−08 | 2.8E−06 | 7.6E−04 |
| Cells_Mean_LargeLipidObjects_Granularity_8_Lipid | 2.1E−07 | 4.8E−08 | 8.6E−06 | 1.7E−03 |
| Cells_Mean_LargeLipidObjects_Granularity_9_Lipid | 1.9E−07 | 4.7E−08 | 7.1E−05 | 6.2E−03 |
| Cells_RadialDistribution_FracAtD_AGP_1of4 | −4.0E−08 | 1.3E−08 | 2.7E−03 | 2.7E−02 |
| Cells_RadialDistribution_FracAtD_DNA_4of4 | 8.5E−08 | 1.9E−08 | 5.3E−06 | 1.2E−03 |
| Cells_RadialDistribution_FracAtD_Mito_4of4 | 6.3E−08 | 1.6E−08 | 7.7E−05 | 6.5E−03 |
| Cells_RadialDistribution_MeanFrac_DNA_4of4 | 8.8E−08 | 2.1E−08 | 4.1E−05 | 4.4E−03 |
| Cells_RadialDistribution_RadialCV_Lipid_1of4 | 6.1E−08 | 1.6E−08 | 2.0E−04 | 1.2E−02 |
| Cells_Texture_Contrast_Mito_5_01 | 2.2E−07 | 8.5E−08 | 8.1E−03 | 4.9E−02 |
| Cells_Texture_Correlation_AGP_5_02 | −6.1E−08 | 2.3E−08 | 7.9E−03 | 4.8E−02 |
| Cells_Texture_DifferenceEntropy_AGP_20_02 | −1.3E−07 | 4.9E−08 | 6.2E−03 | 4.1E−02 |
| Cells_Texture_DifferenceEntropy_DNA_20_01 | −9.9E−08 | 3.1E−08 | 1.6E−03 | 2.3E−02 |
| Cells_Texture_Entropy_Lipid_20_03 | 1.7E−07 | 6.3E−08 | 5.6E−03 | 4.0E−02 |
| Cells_Texture_InfoMeas1_AGP_20_01 | 7.6E−08 | 2.1E−08 | 3.6E−04 | 1.8E−02 |
| Cells_Texture_InfoMeas1_Lipid_10_01 | 1.3E−07 | 3.2E−08 | 9.8E−05 | 7.7E−03 |
| Cells_Texture_InfoMeas1_DNA_20_02 | 1.1E−07 | 1.7E−08 | 2.1E−10 | 3.9E−07 |
| Cells_Texture_SumAverage_Lipid_20_01 | 2.0E−07 | 7.1E−08 | 4.9E−03 | 3.7E−02 |
| Cells_Texture_SumAverage_DNA_5_02 | −1.1E−07 | 3.3E−08 | 8.5E−04 | 1.9E−02 |
| Cells_Texture_SumEntropy_DNA_20_03 | −9.3E−08 | 3.5E−08 | 8.5E−03 | 5.0E−02 |

PLIN2 (ENSG00000147872)

| | | | | |
|---|---|---|---|---|
| Cells_AreaShape_MeanRadius | 2.5E−06 | 9.3E−07 | 6.6E−03 | 4.3E−02 |
| Cells_AreaShape_MedianRadius | 2.8E−06 | 1.0E−06 | 6.0E−03 | 4.1E−02 |
| Cells_AreaShape_Zernike_6_4 | 9.2E−07 | 2.6E−07 | 3.3E−04 | 1.7E−02 |
| Cells_LipidObjects_AreaShape_Area | 7.6E−06 | 2.4E−06 | 1.5E−03 | 2.3E−02 |
| Cells_Correlation_K_AGP_Mito | 9.0E−06 | 3.1E−06 | 4.5E−03 | 3.5E−02 |
| Cells_Granularity_10_Mito | −3.2E−06 | 7.8E−07 | 5.2E−05 | 5.0E−03 |
| Cells_Granularity_11_AGP | −1.6E−06 | 3.8E−07 | 2.4E−05 | 3.3E−03 |
| Cells_Granularity_11_Mito | −4.3E−06 | 7.0E−07 | 1.0E−09 | 1.5E−06 |
| Cells_Granularity_12_Mito | −3.7E−06 | 7.1E−07 | 2.4E−07 | 1.2E−04 |
| Cells_Granularity_14_Mito | −2.3E−06 | 8.3E−07 | 5.5E−03 | 3.9E−02 |
| Cells_Granularity_5_Lipid | 3.1E−06 | 1.1E−06 | 7.5E−03 | 4.6E−02 |
| Cells_Granularity_7_AGP | −3.2E−06 | 1.1E−06 | 3.4E−03 | 3.1E−02 |
| Cells_Granularity_8_AGP | −3.0E−06 | 1.1E−06 | 7.0E−03 | 4.4E−02 |
| Cells_Intensity_LowerQuartileIntensity_Lipid | 1.1E−05 | 3.6E−06 | 2.7E−03 | 2.7E−02 |
| Cells_Intensity_MADIntensity_AGP | −6.3E−06 | 1.6E−06 | 4.9E−05 | 4.9E−03 |
| Cells_Intensity_MaxIntensity_AGP | −4.6E−06 | 1.2E−06 | 1.8E−04 | 1.1E−02 |
| Cells_Intensity_MeanIntensity_AGP | −7.8E−06 | 1.8E−06 | 1.1E−05 | 2.0E−03 |
| Cells_Intensity_MedianIntensity_AGP | −5.8E−06 | 2.0E−06 | 4.1E−03 | 3.4E−02 |
| Cells_Intensity_MedianIntensity_Lipid | 9.9E−06 | 3.6E−06 | 6.7E−03 | 4.3E−02 |
| Cells_Intensity_MinIntensity_AGP | −5.8E−06 | 1.9E−06 | 2.5E−03 | 2.7E−02 |
| Cells_Intensity_MinIntensity_Lipid | 7.8E−06 | 2.9E−06 | 7.2E−03 | 4.5E−02 |
| Cells_Intensity_MinIntensityEdge_AGP | −7.7E−06 | 2.2E−06 | 3.6E−04 | 1.8E−02 |
| Cells_Intensity_MinIntensityEdge_Lipid | 7.9E−06 | 2.9E−06 | 7.1E−03 | 4.5E−02 |
| Cells_Intensity_StdIntensity_AGP | −5.8E−06 | 1.5E−06 | 8.6E−05 | 7.1E−03 |
| Cells_Intensity_StdIntensityEdge_AGP | −4.8E−06 | 1.5E−06 | 1.1E−03 | 2.0E−02 |
| Cells_Intensity_UpperQuartileIntensity_AGP | −7.4E−06 | 1.8E−06 | 2.5E−05 | 3.3E−03 |
| Cells_LargeLipidObjects_AreaShape_Area | 8.1E−06 | 2.6E−06 | 2.0E−03 | 2.5E−02 |
| Cells_Mean_LargeLipidObjects_Correlation_K_AGP_Mito | 8.7E−06 | 2.9E−06 | 2.7E−03 | 2.7E−02 |
| Cells_Mean_LargeLipidObjects_Correlation_K_Lipid_Mito | 6.6E−06 | 2.4E−06 | 5.4E−03 | 3.9E−02 |
| Cells_Mean_LargeLipidObjects_Correlation_K_Mito_AGP | −6.3E−06 | 2.3E−06 | 6.0E−03 | 4.1E−02 |
| Cells_Mean_LargeLipidObjects_Correlation_Overlap_Mito_AGP | −4.4E−06 | 1.2E−06 | 2.8E−04 | 1.5E−02 |
| Cells_Mean_LargeLipidObjects_Granularity_10_Lipid | 7.0E−06 | 1.9E−06 | 1.8E−04 | 1.1E−02 |
| Cells_Mean_LargeLipidObjects_Granularity_11_Lipid | 5.7E−06 | 1.8E−06 | 1.7E−03 | 2.3E−02 |
| Cells_Mean_LargeLipidObjects_Granularity_12_Lipid | 4.8E−06 | 1.6E−06 | 3.6E−03 | 3.2E−02 |
| Cells_Mean_LargeLipidObjects_Granularity_3_Lipid | 6.6E−06 | 2.0E−06 | 1.0E−03 | 1.9E−02 |
| Cells_Mean_LargeLipidObjects_Granularity_4_Lipid | 7.9E−06 | 1.8E−06 | 1.2E−05 | 2.1E−03 |
| Cells_Mean_LargeLipidObjects_Granularity_5_Lipid | 8.9E−06 | 1.9E−06 | 2.9E−06 | 7.9E−04 |
| Cells_Mean_LargeLipidObjects_Granularity_6_Lipid | 9.4E−06 | 2.0E−06 | 2.4E−06 | 6.9E−04 |
| Cells_Mean_LargeLipidObjects_Granularity_7_Lipid | 9.6E−06 | 2.1E−06 | 6.9E−06 | 1.4E−03 |
| Cells_Mean_LargeLipidObjects_Granularity_8_Lipid | 9.2E−06 | 2.1E−06 | 1.2E−05 | 2.0E−03 |
| Cells_Mean_LargeLipidObjects_Granularity_9_Lipid | 8.0E−06 | 2.1E−06 | 9.7E−05 | 7.7E−03 |
| Cells_RadialDistribution_FracAtD_AGP_1of4 | −1.9E−06 | 5.3E−07 | 3.7E−04 | 1.8E−02 |
| Cells_RadialDistribution_FracAtD_DNA_4of4 | 3.7E−06 | 7.8E−07 | 2.8E−06 | 7.7E−04 |
| Cells_RadialDistribution_FracAtD_Mito_4of4 | 2.1E−06 | 7.3E−07 | 4.5E−03 | 3.6E−02 |
| Cells_RadialDistribution_MeanFrac_DNA_4of4 | 3.8E−06 | 9.0E−07 | 2.5E−05 | 3.4E−03 |
| Cells_RadialDistribution_RadialCV_Lipid_1of4 | 2.0E−06 | 7.1E−07 | 4.0E−03 | 3.3E−02 |
| Cells_Texture_AngularSecondMoment_AGP_5_00 | 9.5E−06 | 2.5E−06 | 1.7E−04 | 1.1E−02 |
| Cells_Texture_Contrast_AGP_20_01 | −4.7E−06 | 1.4E−06 | 7.9E−04 | 1.9E−02 |
| Cells_Texture_DifferenceEntropy_AGP_20_01 | −6.3E−06 | 1.6E−06 | 8.2E−05 | 6.8E−03 |
| Cells_Texture_DifferenceEntropy_DNA_20_01 | −4.2E−06 | 1.4E−06 | 2.0E−03 | 2.4E−02 |
| Cells_Texture_DifferenceVariance_AGP_5_03 | 8.6E−06 | 1.9E−06 | 6.8E−06 | 1.4E−03 |
| Cells_Texture_Entropy_AGP_20_03 | −6.8E−06 | 1.7E−06 | 6.7E−05 | 5.9E−03 |
| Cells_Texture_InfoMeas2_AGP_10_03 | −2.7E−06 | 1.0E−06 | 7.6E−03 | 4.7E−02 |
| Cells_Texture_InfoMeas1_Lipid_10_01 | 5.2E−06 | 1.2E−06 | 1.1E−05 | 2.0E−03 |
| Cells_Texture_InfoMeas1_DNA_20_02 | 4.0E−06 | 6.7E−07 | 2.1E−09 | 2.8E−06 |
| Cells_Texture_InverseDifferenceMoment_AGP_5_03 | 8.4E−06 | 1.8E−06 | 3.8E−06 | 9.5E−04 |
| Cells_Texture_InverseDifferenceMoment_DNA_20_03 | 3.8E−06 | 1.4E−06 | 6.8E−03 | 4.4E−02 |
| Cells_Texture_SumAverage_AGP_20_01 | −7.4E−06 | 1.8E−06 | 4.1E−05 | 4.4E−03 |
| Cells_Texture_SumAverage_DNA_5_02 | −4.5E−06 | 1.4E−06 | 1.6E−03 | 2.3E−02 |
| Cells_Texture_SumEntropy_AGP_20_01 | −6.1E−06 | 1.7E−06 | 2.8E−04 | 1.5E−02 |
| Cells_Texture_SumEntropy_DNA_20_01 | −4.2E−06 | 1.5E−06 | 6.2E−03 | 4.1E−02 |
| Cells_Texture_SumVariance_AGP_20_02 | −3.9E−06 | 1.5E−06 | 8.0E−03 | 4.8E−02 |
| Cells_Texture_Variance_AGP_20_01 | −4.4E−06 | 1.5E−06 | 2.9E−03 | 2.8E−02 |

LIPE (ENSG00000079435)

| | | | | |
|---|---|---|---|---|
| Cells_AreaShape_Zernike_7_5 | 1.9E−06 | 6.3E−07 | 2.2E−03 | 2.5E−02 |
| Cells_LipidObjects_AreaShape_Area | 2.1E−05 | 5.0E−06 | 3.1E−05 | 3.8E−03 |
| Cells_LipidObjects_Intensity_IntegratedIntensity_Lipid | 2.4E−05 | 6.5E−06 | 1.9E−04 | 1.2E−02 |
| Cells_Children_LipidObjects_Count | 1.2E−05 | 4.3E−06 | 4.5E−03 | 3.5E−02 |
| Cells_Children_LargeLipidObjects_Count | 2.2E−05 | 7.1E−06 | 2.3E−03 | 2.5E−02 |
| Cells_Correlation_Correlation_DNA_AGP | −8.9E−06 | 3.3E−06 | 6.0E−03 | 4.1E−02 |
| Cells_Correlation_Correlation_Mito_AGP | −1.5E−05 | 4.7E−06 | 1.1E−03 | 2.0E−02 |
| Cells_Correlation_Correlation_Mito_Lipid | −1.5E−05 | 5.3E−06 | 5.3E−03 | 3.9E−02 |
| Cells_Correlation_K_AGP_Mito | 2.1E−05 | 7.6E−06 | 5.4E−03 | 3.9E−02 |
| Cells_Correlation_K_DNA_Mito | 1.7E−05 | 4.9E−06 | 4.9E−04 | 1.9E−02 |
| Cells_Correlation_Overlap_Mito_AGP | −1.4E−05 | 3.6E−06 | 1.2E−04 | 9.1E−03 |
| Cells_Granularity_10_Mito | −8.4E−06 | 1.7E−06 | 7.2E−07 | 2.8E−04 |
| Cells_Granularity_11_AGP | −3.1E−06 | 9.6E−07 | 1.1E−03 | 2.0E−02 |
| Cells_Granularity_11_Mito | −9.2E−06 | 2.1E−06 | 1.6E−05 | 2.5E−03 |
| Cells_Granularity_12_Mito | −6.3E−06 | 2.2E−06 | 5.3E−03 | 3.9E−02 |
| Cells_Granularity_15_Lipid | 5.4E−06 | 1.6E−06 | 1.1E−03 | 2.0E−02 |
| Cells_Granularity_16_Lipid | 5.0E−06 | 1.7E−06 | 4.1E−03 | 3.4E−02 |
| Cells_Granularity_3_Lipid | 7.4E−06 | 2.6E−06 | 3.6E−03 | 3.2E−02 |
| Cells_Granularity_7_AGP | −7.9E−06 | 2.4E−06 | 8.5E−04 | 1.9E−02 |
| Cells_Granularity_8_AGP | −7.1E−06 | 2.4E−06 | 3.4E−03 | 3.1E−02 |
| Cells_Granularity_9_AGP | −6.5E−06 | 2.4E−06 | 7.7E−03 | 4.7E−02 |
| Cells_Granularity_9_Mito | −6.2E−06 | 2.1E−06 | 2.9E−03 | 2.8E−02 |
| Cells_Intensity_IntegratedIntensity_Lipid | 2.5E−05 | 6.4E−06 | 9.6E−05 | 7.7E−03 |
| Cells_Intensity_IntegratedIntensityEdge_Lipid | 3.0E−05 | 6.1E−06 | 1.1E−06 | 3.8E−04 |
| Cells_Intensity_IntegratedIntensityEdge_Mito | 2.3E−05 | 7.2E−06 | 1.7E−03 | 2.3E−02 |
| Cells_Intensity_LowerQuartileIntensity_Lipid | 3.7E−05 | 5.4E−06 | 5.0E−12 | 1.5E−08 |
| Cells_Intensity_LowerQuartileIntensity_Mito | 2.2E−05 | 7.1E−06 | 2.4E−03 | 2.6E−02 |
| Cells_Intensity_MADIntensity_Lipid | 3.0E−05 | 6.5E−06 | 5.5E−06 | 1.2E−03 |
| Cells_Intensity_MaxIntensityEdge_Mito | 1.9E−05 | 7.2E−06 | 6.9E−03 | 4.4E−02 |
| Cells_Intensity_MeanIntensity_Lipid | 2.3E−05 | 6.6E−06 | 4.1E−04 | 1.8E−02 |
| Cells_Intensity_MeanIntensity_Mito | 1.8E−05 | 6.8E−06 | 7.4E−03 | 4.6E−02 |
| Cells_Intensity_MeanIntensityEdge_Lipid | 2.8E−05 | 6.1E−06 | 6.2E−06 | 1.3E−03 |
| Cells_Intensity_MeanIntensityEdge_Mito | 2.7E−05 | 8.1E−06 | 8.4E−04 | 1.9E−02 |
| Cells_Intensity_MedianIntensity_Lipid | 3.4E−05 | 6.1E−06 | 1.9E−08 | 1.7E−05 |
| Cells_Intensity_MedianIntensity_Mito | 1.9E−05 | 7.0E−06 | 5.7E−03 | 4.0E−02 |
| Cells_Intensity_MinIntensity_AGP | −1.3E−05 | 4.6E−06 | 4.2E−03 | 3.4E−02 |
| Cells_Intensity_MinIntensity_Lipid | 2.5E−05 | 5.3E−06 | 2.9E−06 | 7.9E−04 |
| Cells_Intensity_MinIntensityEdge_AGP | −1.6E−05 | 5.4E−06 | 2.8E−03 | 2.8E−02 |
| Cells_Intensity_MinIntensityEdge_Lipid | 2.5E−05 | 5.3E−06 | 2.4E−06 | 6.8E−04 |
| Cells_Intensity_StdIntensityEdge_Mito | 2.1E−05 | 7.6E−06 | 4.9E−03 | 3.7E−02 |
| Cells_Intensity_UpperQuartileIntensity_Lipid | 2.6E−05 | 7.1E−06 | 2.5E−04 | 1.4E−02 |
| Cells_LargeLipidObjects_AreaShape_Area | 2.3E−05 | 5.9E−06 | 7.2E−05 | 6.2E−03 |
| Cells_LargeLipidObjects_Intensity_IntegratedIntensity_Lipid | 2.5E−05 | 7.0E−06 | 3.2E−04 | 1.6E−02 |
| Cells_Mean_LargeLipidObjects_Correlation_K_AGP_Mito | 2.2E−05 | 6.6E−06 | 9.8E−04 | 1.9E−02 |
| Cells_Mean_LargeLipidObjects_Correlation_K_DNA_Mito | 2.1E−05 | 7.2E−06 | 2.8E−03 | 2.8E−02 |
| Cells_Mean_LargeLipidObjects_Correlation_Overlap_DNA_AGP | −9.3E−06 | 3.2E−06 | 3.3E−03 | 3.1E−02 |
| Cells_Mean_LargeLipidObjects_Correlation_Overlap_Mito_AGP | −1.3E−05 | 2.4E−06 | 7.5E−08 | 5.0E−05 |
| Cells_Mean_LargeLipidObjects_Granularity_1_Lipid | 1.6E−05 | 4.4E−06 | 3.7E−04 | 1.8E−02 |
| Cells_Mean_LargeLipidObjects_Granularity_10_Lipid | 1.3E−05 | 4.7E−06 | 4.7E−03 | 3.6E−02 |
| Cells_Mean_LargeLipidObjects_Granularity_2_Lipid | 1.7E−05 | 4.4E−06 | 8.8E−05 | 7.2E−03 |
| Cells_Mean_LargeLipidObjects_Granularity_3_Lipid | 1.9E−05 | 4.5E−06 | 1.6E−05 | 2.5E−03 |
| Cells_Mean_LargeLipidObjects_Granularity_4_Lipid | 2.0E−05 | 4.6E−06 | 1.7E−05 | 2.6E−03 |
| Cells_Mean_LargeLipidObjects_Granularity_5_Lipid | 2.0E−05 | 5.2E−06 | 1.6E−04 | 1.0E−02 |
| Cells_Mean_LargeLipidObjects_Granularity_6_Lipid | 1.9E−05 | 5.6E−06 | 9.3E−04 | 1.9E−02 |
| Cells_Mean_LargeLipidObjects_Granularity_7_Lipid | 1.8E−05 | 5.7E−06 | 1.9E−03 | 2.4E−02 |
| Cells_Mean_LargeLipidObjects_Granularity_8_Lipid | 1.6E−05 | 5.5E−06 | 3.1E−03 | 3.0E−02 |
| Cells_Mean_LargeLipidObjects_Granularity_9_Lipid | 1.4E−05 | 5.3E−06 | 8.4E−03 | 4.9E−02 |
| Cells_RadialDistribution_FracAtD_DNA_4of4 | 7.5E−06 | 1.9E−06 | 8.8E−05 | 7.2E−03 |
| Cells_RadialDistribution_FracAtD_Mito_4of4 | 5.4E−06 | 1.6E−06 | 6.7E−04 | 1.9E−02 |
| Cells_RadialDistribution_MeanFrac_DNA_4of4 | 7.9E−06 | 2.2E−06 | 2.4E−04 | 1.4E−02 |
| Cells_RadialDistribution_RadialCV_AGP_1of4 | 5.1E−06 | 1.7E−06 | 2.1E−03 | 2.5E−02 |
| Cells_Texture_Contrast_Mito_5_00 | 2.6E−05 | 7.0E−06 | 2.6E−04 | 1.4E−02 |
| Cells_Texture_Correlation_AGP_10_02 | −5.4E−06 | 2.0E−06 | 6.1E−03 | 4.1E−02 |
| Cells_Texture_DifferenceEntropy_DNA_5_02 | −9.5E−06 | 3.3E−06 | 3.9E−03 | 3.3E−02 |
| Cells_Texture_Entropy_Lipid_5_02 | 1.7E−05 | 6.6E−06 | 8.1E−03 | 4.9E−02 |
| Cells_Texture_InfoMeas1_AGP_20_00 | 6.9E−06 | 2.3E−06 | 3.3E−03 | 3.1E−02 |
| Cells_Texture_InfoMeas1_Lipid_5_02 | 1.1E−05 | 3.9E−06 | 7.2E−03 | 4.5E−02 |
| Cells_Texture_InfoMeas1_DNA_10_01 | 8.9E−06 | 2.1E−06 | 1.7E−05 | 2.6E−03 |
| Cells_Texture_InfoMeas1_Mito_5_02 | 9.2E−06 | 3.2E−06 | 3.5E−03 | 3.2E−02 |
| Cells_Texture_SumAverage_Lipid_5_02 | 2.2E−05 | 6.5E−06 | 6.6E−04 | 1.9E−02 |
| Cells_Texture_SumAverage_DNA_5_02 | −1.0E−05 | 3.2E−06 | 1.7E−03 | 2.3E−02 |
| Cells_Texture_SumEntropy_DNA_20_01 | −9.6E−06 | 3.4E−06 | 5.1E−03 | 3.8E−02 |

TIMM22 (ENSG00000177370)

| | | | | |
|---|---|---|---|---|
| Cells_Children_LipidObjects_Count | 1.5E−03 | 3.7E−04 | 4.6E−05 | 4.7E−03 |
| Cells_Correlation_K_AGP_Mito | 3.4E−03 | 6.1E−04 | 3.3E−08 | 2.7E−05 |
| Cells_Correlation_K_DNA_Mito | 2.3E−03 | 4.4E−04 | 1.3E−07 | 7.7E−05 |
| Cells_Correlation_Overlap_DNA_AGP | −7.5E−04 | 2.8E−04 | 7.8E−03 | 4.8E−02 |
| Cells_Correlation_Overlap_DNA_Lipid | −1.6E−03 | 4.8E−04 | 1.1E−03 | 2.0E−02 |
| Cells_Granularity_1_Mito | −1.4E−03 | 3.5E−04 | 8.1E−05 | 6.8E−03 |
| Cells_Granularity_3_Lipid | 7.0E−04 | 2.6E−04 | 6.3E−03 | 4.2E−02 |
| Cells_Granularity_4_Lipid | 1.0E−03 | 2.2E−04 | 4.1E−06 | 9.9E−04 |
| Cells_Granularity_5_Lipid | 1.0E−03 | 2.1E−04 | 2.6E−06 | 7.4E−04 |
| Cells_Intensity_IntegratedIntensity_Mito | 2.3E−03 | 5.9E−04 | 9.5E−05 | 7.6E−03 |
| Cells_Intensity_IntegratedIntensityEdge_DNA | 1.1E−03 | 3.6E−04 | 2.2E−03 | 2.5E−02 |
| Cells_Intensity_IntegratedIntensityEdge_Mito | 3.1E−03 | 7.3E−04 | 2.2E−05 | 3.1E−03 |
| Cells_Intensity_LowerQuartileIntensity_DNA | 1.0E−03 | 3.8E−04 | 6.3E−03 | 4.1E−02 |
| Cells_Intensity_LowerQuartileIntensity_Mito | 2.9E−03 | 7.8E−04 | 2.1E−04 | 1.2E−02 |
| Cells_Intensity_MADIntensity_Mito | 3.3E−03 | 5.2E−04 | 4.0E−10 | 6.8E−07 |
| Cells_Intensity_MassDisplacement_Lipid | −4.3E−04 | 1.6E−04 | 8.3E−03 | 4.9E−02 |
| Cells_Intensity_MassDisplacement_DNA | −3.6E−04 | 1.2E−04 | 1.5E−03 | 2.3E−02 |
| Cells_Intensity_MaxIntensity_Mito | 2.4E−03 | 4.3E−04 | 3.5E−08 | 2.8E−05 |
| Cells_Intensity_MaxIntensityEdge_Mito | 3.5E−03 | 5.5E−04 | 3.4E−10 | 5.9E−07 |
| Cells_Intensity_MeanIntensity_Mito | 3.1E−03 | 6.7E−04 | 4.4E−06 | 1.1E−03 |
| Cells_Intensity_MeanIntensityEdge_DNA | 1.1E−03 | 4.1E−04 | 6.4E−03 | 4.2E−02 |
| Cells_Intensity_MeanIntensityEdge_Mito | 3.5E−03 | 8.5E−04 | 4.6E−05 | 4.7E−03 |
| Cells_Intensity_MedianIntensity_Lipid | 2.6E−03 | 9.8E−04 | 7.2E−03 | 4.5E−02 |
| Cells_Intensity_MedianIntensity_Mito | 3.0E−03 | 7.0E−04 | 2.1E−05 | 3.0E−03 |
| Cells_Intensity_StdIntensity_Mito | 3.0E−03 | 5.1E−04 | 2.1E−09 | 2.8E−06 |
| Cells_Intensity_StdIntensityEdge_DNA | 1.0E−03 | 3.8E−04 | 7.6E−03 | 4.7E−02 |
| Cells_Intensity_StdIntensityEdge_Mito | 3.7E−03 | 5.6E−04 | 9.3E−11 | 1.9E−07 |
| Cells_Intensity_UpperQuartileIntensity_Mito | 3.1E−03 | 6.0E−04 | 3.2E−07 | 1.5E−04 |
| Cells_Mean_LargeLipidObjects_Correlation_K_AGP_Mito | 2.8E−03 | 5.7E−04 | 5.8E−07 | 2.4E−04 |
| Cells_Mean_LargeLipidObjects_Correlation_K_Lipid_Mito | 2.3E−03 | 5.6E−04 | 3.8E−05 | 4.2E−03 |
| Cells_Mean_LargeLipidObjects_Correlation_K_DNA_Mito | 2.1E−03 | 7.3E−04 | 4.5E−03 | 3.6E−02 |
| Cells_Mean_LargeLipidObjects_Correlation_K_Mito_AGP | −1.6E−03 | 6.2E−04 | 7.6E−03 | 4.7E−02 |
| Cells_Mean_LargeLipidObjects_Correlation_Overlap_Mito_AGP | −9.9E−04 | 3.5E−04 | 4.5E−03 | 3.5E−02 |
| Cells_Mean_LargeLipidObjects_Granularity_2_Lipid | 1.6E−03 | 5.8E−04 | 7.2E−03 | 4.5E−02 |
| Cells_Mean_LargeLipidObjects_Granularity_3_Lipid | 2.0E−03 | 5.7E−04 | 6.2E−04 | 1.9E−02 |
| Cells_Mean_LargeLipidObjects_Granularity_4_Lipid | 2.1E−03 | 5.5E−04 | 1.2E−04 | 8.9E−03 |
| Cells_Mean_LargeLipidObjects_Granularity_5_Lipid | 2.3E−03 | 5.2E−04 | 6.7E−06 | 1.4E−03 |
| Cells_Mean_LargeLipidObjects_Granularity_6_Lipid | 2.1E−03 | 4.9E−04 | 1.2E−05 | 2.0E−03 |
| Cells_Mean_LargeLipidObjects_Granularity_7_Lipid | 1.7E−03 | 5.5E−04 | 1.7E−03 | 2.3E−02 |
| Cells_RadialDistribution_FracAtD_DNA_4of4 | 5.9E−04 | 2.1E−04 | 5.2E−03 | 3.8E−02 |
| Cells_RadialDistribution_MeanFrac_DNA_4of4 | 6.8E−04 | 2.2E−04 | 2.5E−03 | 2.7E−02 |
| Cells_RadialDistribution_RadialCV_Lipid_1of4 | 8.0E−04 | 1.6E−04 | 3.1E−07 | 1.5E−04 |
| Cells_RadialDistribution_RadialCV_DNA_4of4 | −5.8E−04 | 2.2E−04 | 7.9E−03 | 4.8E−02 |
| Cells_RadialDistribution_RadialCV_Mito_1of4 | 6.0E−04 | 2.0E−04 | 2.9E−03 | 2.8E−02 |
| Cells_Texture_AngularSecondMoment_Mito_20_01 | −1.0E−03 | 3.3E−04 | 2.0E−03 | 2.5E−02 |
| Cells_Texture_Contrast_Mito_5_03 | 3.9E−03 | 5.5E−04 | 3.3E−12 | 1.0E−08 |
| Cells_Texture_Correlation_AGP_5_00 | −7.0E−04 | 2.5E−04 | 4.4E−03 | 3.5E−02 |
| Cells_Texture_DifferenceEntropy_Mito_5_02 | 2.6E−03 | 4.7E−04 | 1.9E−08 | 1.7E−05 |
| Cells_Texture_DifferenceVariance_Mito_10_03 | −1.5E−03 | 5.8E−04 | 8.2E−03 | 4.9E−02 |
| Cells_Texture_Entropy_Lipid_5_00 | 1.8E−03 | 6.5E−04 | 5.3E−03 | 3.9E−02 |
| Cells_Texture_Entropy_Mito_5_00 | 2.5E−03 | 4.6E−04 | 1.2E−07 | 7.1E−05 |
| Cells_Texture_InfoMeas1_Lipid_5_02 | 1.4E−03 | 4.1E−04 | 6.1E−04 | 1.9E−02 |
| Cells_Texture_InfoMeas1_Mito_5_00 | 8.6E−04 | 3.1E−04 | 6.2E−03 | 4.1E−02 |
| Cells_Texture_InverseDifferenceMoment_Lipid_5_02 | −1.8E−03 | 5.8E−04 | 1.8E−03 | 2.3E−02 |
| Cells_Texture_InverseDifferenceMoment_Mito_20_01 | −1.5E−03 | 4.1E−04 | 2.1E−04 | 1.3E−02 |
| Cells_Texture_SumAverage_Mito_5_02 | 3.1E−03 | 6.5E−04 | 2.6E−06 | 7.3E−04 |
| Cells_Texture_SumEntropy_Mito_5_02 | 2.4E−03 | 4.5E−04 | 8.9E−08 | 5.7E−05 |
| Cells_Texture_SumVariance_Mito_5_00 | 3.9E−03 | 6.5E−04 | 2.4E−09 | 3.2E−06 |
| Cells_Texture_Variance_Mito_5_00 | 3.9E−03 | 6.2E−04 | 3.5E−10 | 6.1E−07 |

INSR (ENSG00000171105)

| | | | | |
|---|---|---|---|---|
| Cells_AreaShape_MaximumRadius | 1.2E−04 | 4.2E−05 | 3.5E−03 | 3.2E−02 |
| Cells_AreaShape_MeanRadius | 1.5E−04 | 4.1E−05 | 3.8E−04 | 1.8E−02 |
| Cells_AreaShape_MedianRadius | 1.7E−04 | 4.5E−05 | 2.2E−04 | 1.3E−02 |
| Cells_LipidObjects_AreaShape_Area | 4.7E−04 | 1.1E−04 | 8.1E−06 | 1.6E−03 |
| Cells_LipidObjects_Intensity_IntegratedIntensity_Lipid | 5.6E−04 | 1.4E−04 | 3.9E−05 | 4.2E−03 |
| Cells_Children_LipidObjects_Count | 2.8E−04 | 9.4E−05 | 2.7E−03 | 2.7E−02 |
| Cells_Children_LargeLipidObjects_Count | 4.6E−04 | 1.6E−04 | 4.0E−03 | 3.3E−02 |
| Cells_Correlation_Correlation_Mito_AGP | −3.0E−04 | 1.1E−04 | 4.9E−03 | 3.7E−02 |
| Cells_Correlation_Correlation_Mito_Lipid | −3.4E−04 | 1.1E−04 | 2.3E−03 | 2.6E−02 |
| Cells_Correlation_K_DNA_Mito | 3.5E−04 | 1.3E−04 | 5.2E−03 | 3.8E−02 |
| Cells_Correlation_K_Mito_DNA | −2.3E−04 | 8.6E−05 | 7.9E−03 | 4.8E−02 |
| Cells_Correlation_Overlap_Mito_AGP | −2.8E−04 | 8.7E−05 | 1.2E−03 | 2.2E−02 |
| Cells_Granularity_10_Mito | −1.9E−04 | 4.4E−05 | 1.2E−05 | 2.0E−03 |
| Cells_Granularity_11_AGP | −6.9E−05 | 2.1E−05 | 1.0E−03 | 1.9E−02 |
| Cells_Granularity_11_Mito | −2.5E−04 | 4.8E−05 | 1.7E−07 | 9.6E−05 |
| Cells_Granularity_12_Mito | −1.8E−04 | 4.9E−05 | 1.5E−04 | 1.0E−02 |
| Cells_Granularity_14_Lipid | 1.0E−04 | 3.6E−05 | 4.6E−03 | 3.6E−02 |
| Cells_Granularity_15_Lipid | 1.0E−04 | 3.8E−05 | 6.4E−03 | 4.2E−02 |
| Cells_Granularity_7_AGP | −1.7E−04 | 5.2E−05 | 1.5E−03 | 2.3E−02 |
| Cells_Granularity_8_AGP | −1.7E−04 | 5.2E−05 | 1.2E−03 | 2.2E−02 |
| Cells_Granularity_9_AGP | −1.6E−04 | 5.3E−05 | 2.6E−03 | 2.7E−02 |
| Cells_Intensity_IntegratedIntensity_Lipid | 5.7E−04 | 1.3E−04 | 1.6E−05 | 2.4E−03 |
| Cells_Intensity_IntegratedIntensityEdge_Lipid | 5.7E−04 | 1.5E−04 | 1.3E−04 | 9.1E−03 |
| Cells_Intensity_LowerQuartileIntensity_Lipid | 6.3E−04 | 1.7E−04 | 1.5E−04 | 9.9E−03 |
| Cells_Intensity_MADIntensity_Lipid | 6.4E−04 | 1.5E−04 | 1.3E−05 | 2.2E−03 |
| Cells_Intensity_MeanIntensity_Lipid | 4.8E−04 | 1.5E−04 | 1.3E−03 | 2.2E−02 |
| Cells_Intensity_MeanIntensityEdge_Lipid | 5.4E−04 | 1.5E−04 | 2.8E−04 | 1.5E−02 |
| Cells_Intensity_MedianIntensity_Lipid | 6.9E−04 | 1.5E−04 | 4.5E−06 | 1.1E−03 |
| Cells_Intensity_MinIntensity_AGP | −2.9E−04 | 1.0E−04 | 4.6E−03 | 3.6E−02 |
| Cells_Intensity_MinIntensity_Lipid | 4.8E−04 | 1.3E−04 | 2.7E−04 | 1.5E−02 |
| Cells_Intensity_MinIntensityEdge_AGP | −3.5E−04 | 1.2E−04 | 3.2E−03 | 3.0E−02 |
| Cells_Intensity_MinIntensityEdge_Lipid | 4.8E−04 | 1.3E−04 | 2.7E−04 | 1.5E−02 |
| Cells_Intensity_UpperQuartileIntensity_Lipid | 5.7E−04 | 1.5E−04 | 1.9E−04 | 1.2E−02 |
| Cells_LargeLipidObjects_AreaShape_Area | 5.4E−04 | 1.3E−04 | 2.3E−05 | 3.1E−03 |
| Cells_LargeLipidObjects_Intensity_IntegratedIntensity_Lipid | 6.0E−04 | 1.5E−04 | 4.3E−05 | 4.6E−03 |
| Cells_Mean_LargeLipidObjects_Correlation_K_DNA_Mito | 4.4E−04 | 1.7E−04 | 8.3E−03 | 4.9E−02 |
| Cells_Mean_LargeLipidObjects_Correlation_Overlap_DNA_AGP | −2.3E−04 | 6.8E−05 | 7.4E−04 | 1.9E−02 |
| Cells_Mean_LargeLipidObjects_Correlation_Overlap_Mito_AGP | −2.9E−04 | 5.4E−05 | 6.5E−08 | 4.5E−05 |
| Cells_Mean_LargeLipidObjects_Granularity_1_Lipid | 3.2E−04 | 1.1E−04 | 3.7E−03 | 3.3E−02 |
| Cells_Mean_LargeLipidObjects_Granularity_10_Lipid | 3.3E−04 | 9.4E−05 | 3.8E−04 | 1.8E−02 |
| Cells_Mean_LargeLipidObjects_Granularity_2_Lipid | 3.3E−04 | 1.1E−04 | 3.1E−03 | 2.9E−02 |
| Cells_Mean_LargeLipidObjects_Granularity_3_Lipid | 3.9E−04 | 1.1E−04 | 4.9E−04 | 1.9E−02 |
| Cells_Mean_LargeLipidObjects_Granularity_4_Lipid | 4.2E−04 | 1.1E−04 | 2.0E−04 | 1.2E−02 |
| Cells_Mean_LargeLipidObjects_Granularity_5_Lipid | 4.4E−04 | 1.2E−04 | 1.8E−04 | 1.1E−02 |
| Cells_Mean_LargeLipidObjects_Granularity_6_Lipid | 4.6E−04 | 1.1E−04 | 5.7E−05 | 5.4E−03 |
| Cells_Mean_LargeLipidObjects_Granularity_7_Lipid | 4.6E−04 | 1.1E−04 | 3.0E−05 | 3.7E−03 |
| Cells_Mean_LargeLipidObjects_Granularity_8_Lipid | 4.3E−04 | 1.1E−04 | 4.8E−05 | 4.8E−03 |
| Cells_Mean_LargeLipidObjects_Granularity_9_Lipid | 3.8E−04 | 1.0E−04 | 2.4E−04 | 1.4E−02 |
| Cells_RadialDistribution_FracAtD_DNA_4of4 | 1.8E−04 | 4.2E−05 | 2.4E−05 | 3.3E−03 |
| Cells_RadialDistribution_FracAtD_Mito_4of4 | 1.2E−04 | 3.8E−05 | 2.5E−03 | 2.6E−02 |
| Cells_RadialDistribution_MeanFrac_DNA_4of4 | 1.9E−04 | 4.6E−05 | 2.9E−05 | 3.7E−03 |
| Cells_RadialDistribution_RadialCV_Lipid_1of4 | 1.3E−04 | 3.9E−05 | 7.3E−04 | 1.9E−02 |
| Cells_Texture_Contrast_DNA_20_01 | −2.5E−04 | 9.1E−05 | 5.9E−03 | 4.0E−02 |
| Cells_Texture_Correlation_Lipid_20_03 | 1.1E−04 | 3.6E−05 | 1.5E−03 | 2.3E−02 |
| Cells_Texture_DifferenceEntropy_DNA_5_02 | −2.3E−04 | 6.8E−05 | 8.2E−04 | 1.9E−02 |
| Cells_Texture_Entropy_Lipid_20_03 | 3.9E−04 | 1.3E−04 | 3.4E−03 | 3.1E−02 |
| Cells_Texture_Entropy_DNA_20_03 | −2.1E−04 | 7.5E−05 | 5.5E−03 | 3.9E−02 |
| Cells_Texture_InfoMeas1_AGP_20_01 | 1.5E−04 | 5.0E−05 | 2.5E−03 | 2.6E−02 |
| Cells_Texture_InfoMeas1_Lipid_10_01 | 3.1E−04 | 7.4E−05 | 3.7E−05 | 4.1E−03 |
| Cells_Texture_InfoMeas1_DNA_20_02 | 2.6E−04 | 4.3E−05 | 1.0E−09 | 1.6E−06 |
| Cells_Texture_InverseDifferenceMoment_DNA_20_01 | 1.9E−04 | 6.7E−05 | 3.8E−03 | 3.3E−02 |
| Cells_Texture_SumAverage_Lipid_20_01 | 4.4E−04 | 1.5E−04 | 2.6E−03 | 2.7E−02 |
| Cells_Texture_SumAverage_DNA_5_02 | −2.9E−04 | 7.0E−05 | 3.5E−05 | 3.9E−03 |
| Cells_Texture_SumEntropy_DNA_20_01 | −2.3E−04 | 7.1E−05 | 1.4E−03 | 2.2E−02 |
| Cells_Texture_SumVariance_DNA_5_02 | −2.9E−04 | 9.2E−05 | 1.9E−03 | 2.4E−02 |
| Cells_Texture_Variance_DNA_5_01 | −2.8E−04 | 9.2E−05 | 2.2E−03 | 2.5E−02 |

GLUTA (ENSG00000181856)

| | | | | |
|---|---|---|---|---|
| Cells_AreaShape_Zernike_6_4 | 8.1E−06 | 1.7E−06 | 1.8E−06 | 5.5E−04 |
| Cells_LipidObjects_AreaShape_Area | 5.9E−05 | 1.8E−05 | 1.1E−03 | 2.0E−02 |
| Cells_LipidObjects_Intensity_IntegratedIntensity_Lipid | 6.2E−05 | 2.2E−05 | 6.1E−03 | 4.1E−02 |
| Cells_Correlation_Correlation_DNA_AGP | −2.7E−05 | 9.7E−06 | 4.9E−03 | 3.7E−02 |
| Cells_Correlation_Overlap_Mito_AGP | −3.7E−05 | 1.2E−05 | 2.5E−03 | 2.6E−02 |
| Cells_Granularity_10_Mito | −2.3E−05 | 6.3E−06 | 2.9E−04 | 1.5E−02 |
| Cells_Granularity_11_AGP | −1.2E−05 | 3.0E−06 | 9.4E−05 | 7.5E−03 |
| Cells_Granularity_11_Mito | −3.0E−05 | 6.3E−06 | 2.3E−06 | 6.7E−04 |
| Cells_Granularity_12_Mito | −2.6E−05 | 5.8E−06 | 6.1E−06 | 1.3E−03 |
| Cells_Granularity_7_AGP | −2.5E−05 | 8.3E−06 | 2.4E−03 | 2.6E−02 |
| Cells_Granularity_8_AGP | −2.4E−05 | 8.4E−06 | 4.8E−03 | 3.6E−02 |
| Cells_Intensity_IntegratedIntensity_Lipid | 6.9E−05 | 2.5E−05 | 5.4E−03 | 3.9E−02 |
| Cells_Intensity_LowerQuartileIntensity_Lipid | 8.2E−05 | 2.9E−05 | 4.4E−03 | 3.5E−02 |
| Cells_Intensity_MADIntensity_AGP | −4.2E−05 | 1.3E−05 | 1.2E−03 | 2.1E−02 |
| Cells_Intensity_MADIntensity_Lipid | 7.5E−05 | 2.7E−05 | 4.7E−03 | 3.6E−02 |
| Cells_Intensity_MaxIntensity_AGP | −3.1E−05 | 1.0E−05 | 3.2E−03 | 3.0E−02 |
| Cells_Intensity_MeanIntensity_AGP | −4.4E−05 | 1.5E−05 | 4.3E−03 | 3.5E−02 |
| Cells_Intensity_MedianIntensity_AGP | −4.3E−05 | 1.6E−05 | 6.0E−03 | 4.1E−02 |
| Cells_Intensity_MedianIntensity_Lipid | 8.4E−05 | 2.8E−05 | 2.4E−03 | 2.6E−02 |
| Cells_Intensity_MinIntensity_AGP | −4.8E−05 | 1.4E−05 | 5.5E−04 | 1.9E−02 |
| Cells_Intensity_MinIntensity_Lipid | 6.7E−05 | 2.2E−05 | 2.4E−03 | 2.6E−02 |
| Cells_Intensity_MinIntensityEdge_AGP | −6.2E−05 | 1.6E−05 | 8.7E−05 | 7.2E−03 |
| Cells_Intensity_MinIntensityEdge_Lipid | 6.7E−05 | 2.2E−05 | 2.4E−03 | 2.6E−02 |
| Cells_Intensity_StdIntensity_AGP | −4.0E−05 | 1.2E−05 | 8.7E−04 | 1.9E−02 |
| Cells_Intensity_StdIntensityEdge_AGP | −3.2E−05 | 1.2E−05 | 6.4E−03 | 4.2E−02 |
| Cells_Intensity_UpperQuartileIntensity_AGP | −4.6E−05 | 1.5E−05 | 1.6E−03 | 2.3E−02 |
| Cells_LargeLipidObjects_AreaShape_Area | 6.4E−05 | 2.0E−05 | 1.7E−03 | 2.3E−02 |
| Cells_LargeLipidObjects_Intensity_IntegratedIntensity_Lipid | 6.2E−05 | 2.3E−05 | 6.9E−03 | 4.4E−02 |
| Cells_Mean_LargeLipidObjects_AreaShape_Orientation | 1.1E−06 | 4.0E−07 | 7.1E−03 | 4.5E−02 |
| Cells_Mean_LargeLipidObjects_Correlation_K_AGP_Mito | 6.0E−05 | 2.3E−05 | 7.6E−03 | 4.7E−02 |
| Cells_Mean_LargeLipidObjects_Correlation_K_Mito_AGP | −4.9E−05 | 1.7E−05 | 4.3E−03 | 3.5E−02 |
| Cells_Mean_LargeLipidObjects_Correlation_Overlap_DNA_AGP | −3.2E−05 | 9.7E−06 | 1.1E−03 | 2.0E−02 |
| Cells_Mean_LargeLipidObjects_Correlation_Overlap_Mito_AGP | −3.5E−05 | 9.0E−06 | 7.9E−05 | 6.7E−03 |
| Cells_Mean_LargeLipidObjects_Granularity_1_Lipid | 4.1E−05 | 1.5E−05 | 5.3E−03 | 3.9E−02 |
| Cells_Mean_LargeLipidObjects_Granularity_10_Lipid | 4.9E−05 | 1.5E−05 | 1.5E−03 | 2.2E−02 |
| Cells_Mean_LargeLipidObjects_Granularity_3_Lipid | 5.3E−05 | 1.5E−05 | 4.4E−04 | 1.8E−02 |
| Cells_Mean_LargeLipidObjects_Granularity_4_Lipid | 5.9E−05 | 1.4E−05 | 2.6E−05 | 3.5E−03 |
| Cells_Mean_LargeLipidObjects_Granularity_5_Lipid | 6.6E−05 | 1.5E−05 | 6.0E−06 | 1.3E−03 |
| Cells_Mean_LargeLipidObjects_Granularity_6_Lipid | 6.8E−05 | 1.6E−05 | 1.2E−05 | 2.0E−03 |
| Cells_Mean_LargeLipidObjects_Granularity_7_Lipid | 6.7E−05 | 1.7E−05 | 9.9E−05 | 7.8E−03 |
| Cells_Mean_LargeLipidObjects_Granularity_8_Lipid | 6.3E−05 | 1.7E−05 | 3.2E−04 | 1.6E−02 |
| Cells_Mean_LargeLipidObjects_Granularity_9_Lipid | 5.5E−05 | 1.7E−05 | 1.2E−03 | 2.1E−02 |
| Cells_RadialDistribution_FracAtD_AGP_4of4 | 1.5E−05 | 5.0E−06 | 2.0E−03 | 2.5E−02 |
| Cells_RadialDistribution_FracAtD_DNA_4of4 | 2.6E−05 | 6.0E−06 | 1.7E−05 | 2.5E−03 |
| Cells_RadialDistribution_FracAtD_Mito_4of4 | 1.8E−05 | 5.3E−06 | 9.6E−04 | 1.9E−02 |
| Cells_RadialDistribution_MeanFrac_AGP_4of4 | 2.6E−05 | 9.5E−06 | 6.1E−03 | 4.1E−02 |
| Cells_RadialDistribution_MeanFrac_DNA_4of4 | 2.6E−05 | 6.9E−06 | 1.6E−04 | 1.0E−02 |
| Cells_RadialDistribution_RadialCV_AGP_1of4 | 1.6E−05 | 5.3E−06 | 3.2E−03 | 3.0E−02 |
| Cells_Radial Distribution_RadialCV_Lipid_1of4 | 1.6E−05 | 5.2E−06 | 3.1E−03 | 3.0E−02 |
| Cells_Texture_AngularSecondMoment_AGP_5_00 | 7.4E−05 | 1.9E−05 | 1.3E−04 | 9.5E−03 |
| Cells_Texture_Contrast_AGP_20_01 | −3.1E−05 | 1.2E−05 | 7.5E−03 | 4.6E−02 |
| Cells_Texture_Correlation_Lipid_5_00 | 2.7E−05 | 9.1E−06 | 3.4E−03 | 3.1E−02 |
| Cells_Texture_DifferenceEntropy_AGP_20_01 | −4.6E−05 | 1.3E−05 | 3.2E−04 | 1.6E−02 |
| Cells_Texture_DifferenceVariance_AGP_20_00 | 6.6E−05 | 1.5E−05 | 6.1E−06 | 1.3E−03 |
| Cells_Texture_Entropy_AGP_20_03 | −4.9E−05 | 1.4E−05 | 3.8E−04 | 1.8E−02 |
| Cells_Texture_InfoMeas1_AGP_20_01 | 2.1E−05 | 7.2E−06 | 3.5E−03 | 3.2E−02 |
| Cells_Texture_InfoMeas1_Lipid_10_01 | 3.5E−05 | 9.9E−06 | 4.0E−04 | 1.8E−02 |
| Cells_Texture_InfoMeas1_DNA_20_02 | 2.6E−05 | 6.6E−06 | 8.4E−05 | 7.0E−03 |
| Cells_Texture_InverseDifferenceMoment_AGP_10_00 | 6.3E−05 | 1.4E−05 | 6.6E−06 | 1.4E−03 |
| Cells_Texture_InverseDifferenceMoment_DNA_20_01 | 2.9E−05 | 1.1E−05 | 8.1E−03 | 4.9E−02 |
| Cells_Texture_SumAverage_AGP_20_03 | −4.5E−05 | 1.5E−05 | 3.0E−03 | 2.9E−02 |
| Cells_Texture_SumEntropy_AGP_20_01 | −4.4E−05 | 1.3E−05 | 1.1E−03 | 2.0E−02 |
| Cells_Texture_SumVariance_AGP_5_03 | −3.0E−05 | 1.1E−05 | 7.5E−03 | 4.6E−02 |
| Cells_Texture_Variance_AGP_20_02 | −3.1E−05 | 1.2E−05 | 7.9E−03 | 4.8E−02 |

TABLE 3
Significant effects of drug perturbation on LP-profiles in AMSCs and PHH (t−test,
significance level AMSCs 5% FDR, PHH 0.1% FDR). p-value of t-test, q-value of t-test,
t-statistics of t-test

| | | | |
|---|---|---|---|
| LipocyteProfiler features | p-value | q-value | t-statistics |

mature visceral AMSCs isoproterenol treatment

| | | | |
|---|---|---|---|
| Cells_AreaShape_Eccentricity | 0.0132 | 0.0334 | −0.5969 |
| Cells_AreaShape_MaximumRadius | 0.0274 | 0.0480 | 1.0800 |
| Cells_AreaShape_Zernike_2_0 | 0.0158 | 0.0372 | 1.0184 |
| Cells_Correlation_Correlation_DNA_Lipid | 0.0172 | 0.0390 | 0.5553 |
| Cells_Correlation_K_Lipid_AGP | 0.0000 | 0.0016 | 1.0663 |
| Cells_Correlation_K_Lipid_DNA | 0.0011 | 0.0159 | 0.7035 |
| Cells_Granularity_11_Lipid | 0.0121 | 0.0324 | −0.8778 |
| Cells_Granularity_9_AGP | 0.0253 | 0.0464 | −0.9607 |
| Cells_Granularity_6_Lipid | 0.0243 | 0.0457 | −1.1015 |
| Cells_Granularity_8_Mito | 0.0229 | 0.0448 | −1.0998 |
| Cells_Intensity_MassDisplacement_DNA | 0.0118 | 0.0322 | 1.3798 |
| Cells_Intensity_MassDisplacement_Mito | 0.0054 | 0.0251 | 0.5479 |
| Cells_Intensity_MaxIntensity_Lipid | 0.0024 | 0.0193 | −0.4797 |
| Cells_Intensity_MeanIntensityEdge_DNA | 0.0188 | 0.0404 | −0.6042 |
| Cells_Intensity_StdIntensityEdge_DNA | 0.0118 | 0.0322 | −0.6431 |
| Cells_Mean_LargeLipidObjects_AreaShape_Eccentricity | 0.0051 | 0.0247 | 0.8529 |
| Cells_Mean_LargeLipidObjects_AreaShape_FormFactor | 0.0201 | 0.0417 | −0.5459 |
| Cells_Mean_LargeLipidObjects_AreaShape_Solidity | 0.0165 | 0.0381 | −0.5118 |
| Cells_Mean_LargeLipidObjects_Correlation_K_Mito_Lipid | 0.0106 | 0.0310 | −0.4755 |
| Cells_Mean_LargeLipidObjects_Correlation_Overlap_DNA_AGP | 0.0121 | 0.0324 | 1.0958 |
| Cells_Mean_LargeLipidObjects_Correlation_Overlap_Mito_AGP | 0.0097 | 0.0303 | 1.6234 |
| Cells_RadialDistribution_FracAtD_DNA_3of4 | 0.0261 | 0.0470 | 0.5310 |
| Cells_RadialDistribution_MeanFrac_Lipid_1of4 | 0.0128 | 0.0328 | −1.2714 |
| Cells_RadialDistribution_RadialCV_AGP_4of4 | 0.0122 | 0.0324 | −1.1200 |
| Cells_Texture_Correlation_AGP_10_00 | 0.0100 | 0.0307 | 0.7054 |
| Cells_Texture_Correlation_DNA_20_02 | 0.0124 | 0.0324 | 1.0506 |
| Cells_Texture_Correlation_Mito_20_00 | 0.0248 | 0.0462 | 0.7498 |
| Cells_Texture_DifferenceEntropy_AGP_10_01 | 0.0269 | 0.0475 | −0.6327 |
| Cells_Texture_DifferenceEntropy_Lipid_10_03 | 0.0032 | 0.0197 | −0.7727 |
| Cells_Texture_DifferenceVariance_Lipid_5_02 | 0.0001 | 0.0028 | 1.0550 |
| Cells_Texture_Entropy_Lipid_10_00 | 0.0084 | 0.0285 | −0.7719 |
| Cells_Texture_InfoMeas2_Lipid_20_01 | 0.0112 | 0.0317 | −0.6491 |
| Cells_Texture_InfoMeas1_DNA_10_01 | 0.0260 | 0.0470 | −0.9901 |
| Cells_Texture_InfoMeas1_Mito_10_01 | 0.0059 | 0.0253 | −1.0813 |
| Cells_Texture_InverseDifferenceMoment_Lipid_5_00 | 0.0176 | 0.0395 | 0.8646 |
| Cells_Texture_SumAverage_DNA_20_02 | 0.0199 | 0.0417 | −0.3251 |
| Cells_Texture_SumEntropy_Lipid_10_00 | 0.0057 | 0.0251 | −0.7100 |
| Cytoplasm_AreaShape_Compactness | 0.0007 | 0.0118 | −0.9665 |
| Cytoplasm_AreaShape_Eccentricity | 0.0255 | 0.0464 | −0.5269 |
| Cytoplasm_AreaShape_MaximumRadius | 0.0269 | 0.0475 | 1.0914 |
| Cytoplasm_AreaShape_MeanRadius | 0.0183 | 0.0400 | 1.2378 |
| Cytoplasm_AreaShape_Zernike_5_1 | 0.0074 | 0.0269 | 1.3893 |
| Cytoplasm_Correlation_K_Lipid_AGP | 0.0106 | 0.0310 | 1.1667 |
| Cytoplasm_Granularity_11_Lipid | 0.0092 | 0.0296 | −0.9346 |
| Cytoplasm_Granularity_9_AGP | 0.0215 | 0.0434 | −0.9875 |
| Cytoplasm_Granularity_6_Lipid | 0.0213 | 0.0432 | −1.1339 |
| Cytoplasm_Granularity_8_Mito | 0.0250 | 0.0462 | −1.2135 |
| Cytoplasm_Intensity_MassDisplacement_Mito | 0.0024 | 0.0193 | 0.5680 |
| Cytoplasm_Intensity_MaxIntensity_Lipid | 0.0017 | 0.0178 | −0.4964 |
| Cytoplasm_RadialDistribution_FracAtD_AGP_1of4 | 0.0068 | 0.0268 | 1.3822 |
| Cytoplasm_RadialDistribution_FracAtD_DNA_2of4 | 0.0007 | 0.0118 | 0.9333 |
| Cytoplasm_RadialDistribution_FracAtD_Mito_1of4 | 0.0262 | 0.0470 | 1.2949 |
| Cytoplasm_RadialDistribution_MeanFrac_AGP_2of4 | 0.0169 | 0.0386 | 1.1326 |
| Cytoplasm_Texture_Correlation_AGP_5_03 | 0.0290 | 0.0498 | 1.1195 |
| Cytoplasm_Texture_Correlation_DNA_20_03 | 0.0093 | 0.0296 | 0.6501 |
| Cytoplasm_Texture_Correlation_Mito_20_01 | 0.0023 | 0.0193 | 0.9141 |
| Cytoplasm_Texture_DifferenceEntropy_Lipid_20_00 | 0.0031 | 0.0197 | −0.7826 |
| Cytoplasm_Texture_DifferenceVariance_Lipid_10_03 | 0.0000 | 0.0015 | 1.2846 |
| Cytoplasm_Texture_Entropy_Lipid_10_00 | 0.0289 | 0.0498 | −0.6219 |
| Cytoplasm_Texture_InfoMeas2_Lipid_20_01 | 0.0004 | 0.0070 | −0.6576 |
| Cytoplasm_Texture_InfoMeas1_DNA_5_00 | 0.0039 | 0.0221 | −0.6451 |
| Cytoplasm_Texture_InfoMeas1_Mito_10_01 | 0.0018 | 0.0178 | −1.4854 |
| Cytoplasm_Texture_SumEntropy_Lipid_20_00 | 0.0100 | 0.0307 | −0.6528 |
| Nuclei_AreaShape_Eccentricity | 0.0018 | 0.0178 | −1.0867 |
| Nuclei_AreaShape_Zernike_3_3 | 0.0290 | 0.0498 | −1.2983 |
| Nuclei_Correlation_Correlation_Lipid_AGP | 0.0260 | 0.0470 | 1.0582 |
| Nuclei_Correlation_Correlation_DNA_Lipid | 0.0017 | 0.0178 | 0.6578 |
| Nuclei_Correlation_K_Lipid_AGP | 0.0152 | 0.0365 | 0.2224 |
| Nuclei_Correlation_Overlap_Lipid_AGP | 0.0053 | 0.0251 | 0.5245 |
| Nuclei_Correlation_Overlap_DNA_AGP | 0.0027 | 0.0197 | 1.2900 |
| Nuclei_Correlation_Overlap_DNA_Lipid | 0.0074 | 0.0269 | 0.5311 |
| Nuclei_Correlation_Overlap_Mito_Lipid | 0.0078 | 0.0273 | 0.6243 |
| Nuclei_Granularity_11_Lipid | 0.0135 | 0.0338 | −0.7548 |
| Nuclei_Granularity_11_Mito | 0.0159 | 0.0372 | 0.3324 |
| Nuclei_Granularity_9_AGP | 0.0163 | 0.0380 | −1.2602 |
| Nuclei_Granularity_1_Lipid | 0.0015 | 0.0178 | 1.0348 |
| Nuclei_Granularity_4_DNA | 0.0095 | 0.0302 | −1.0986 |
| Nuclei_Intensity_MassDisplacement_AGP | 0.0021 | 0.0185 | −1.0493 |
| Nuclei_Intensity_MinIntensity_DNA | 0.0046 | 0.0236 | 0.3131 |
| Nuclei_Intensity_MinIntensityEdge_DNA | 0.0045 | 0.0236 | 0.3134 |
| Nuclei_RadialDistribution_FracAtD_Lipid_1of4 | 0.0157 | 0.0371 | 1.0255 |
| Nuclei_RadialDistribution_MeanFrac_Lipid_1of4 | 0.0278 | 0.0484 | 0.7832 |
| Nuclei_RadialDistribution_MeanFrac_DNA_3of4 | 0.0014 | 0.0178 | −1.2471 |
| Nuclei_RadialDistribution_RadialCV_AGP_3of4 | 0.0057 | 0.0251 | −1.3273 |
| Nuclei_RadialDistribution_RadialCV_DNA_3of4 | 0.0182 | 0.0400 | −1.2101 |
| Nuclei_Texture_Correlation_Lipid_5_01 | 0.0053 | 0.0251 | −1.6472 |
| Nuclei_Texture_Correlation_DNA_5_02 | 0.0118 | 0.0322 | −0.9491 |
| Nuclei_Texture_Correlation_Mito_5_02 | 0.0066 | 0.0263 | −0.8419 |
| Nuclei_Texture_DifferenceEntropy_Lipid_20_03 | 0.0054 | 0.0251 | −0.8156 |
| Nuclei_Texture_DifferenceVariance_Lipid_20_03 | 0.0212 | 0.0432 | 0.4696 |
| Nuclei_Texture_Entropy_Lipid_20_01 | 0.0055 | 0.0251 | −0.7635 |
| Nuclei_Texture_InfoMeas2_AGP_5_02 | 0.0023 | 0.0193 | −1.1995 |
| Nuclei_Texture_InfoMeas1_Lipid_5_01 | 0.0101 | 0.0308 | 1.0781 |
| Nuclei_Texture_InfoMeas2_DNA_5_02 | 0.0040 | 0.0221 | −0.8347 |
| Nuclei_Texture_InverseDifferenceMoment_Lipid_20_00 | 0.0008 | 0.0126 | 0.7428 |
| Nuclei_Texture_SumAverage_DNA_20_03 | 0.0028 | 0.0197 | 0.4538 |
| Nuclei_Texture_SumEntropy_Lipid_20_01 | 0.0063 | 0.0261 | −0.7807 |

PHH olic acid treatment

| | | | |
|---|---|---|---|
| Cells_Children_LargeLipidObjects_Count | 2.3E−11 | 3.4E−10 | 1.9E+01 |
| Cells_Correlation_Correlation_Lipid_AGP | 9.5E−13 | 7.7E−11 | −2.4E+01 |
| Cells_Correlation_Correlation_DNA_Lipid | 2.1E−12 | 1.2E−10 | −2.3E+01 |
| Cells_Correlation_Correlation_Mito_Lipid | 5.1E−06 | 1.6E−05 | −7.1E+00 |
| Cells_Correlation_K_AGP_Lipid | 2.6E−07 | 1.0E−06 | 9.2E+00 |
| Cells_Correlation_K_Lipid_AGP | 2.0E−11 | 3.1E−10 | −1.9E+01 |
| Cells_Correlation_K_Lipid_DNA | 2.2E−11 | 3.3E−10 | −1.9E+01 |
| Cells_Correlation_K_Lipid_Mito | 1.2E−08 | 5.5E−08 | −1.2E+01 |
| Cells_Correlation_K_DNA_Lipid | 4.1E−07 | 1.5E−06 | 8.9E+00 |
| Cells_Correlation_K_DNA_Mito | 1.2E−04 | 2.9E−04 | 5.2E+00 |
| Cells_Correlation_K_Mito_Lipid | 8.1E−09 | 3.7E−08 | 1.2E+01 |
| Cells_Correlation_Overlap_Lipid_AGP | 1.3E−11 | 2.3E−10 | −2.0E+01 |
| Cells_Correlation_Overlap_DNA_Lipid | 9.0E−12 | 2.1E−10 | −2.0E+01 |
| Cells_Correlation_Overlap_Mito_Lipid | 2.1E−08 | 9.0E−08 | −1.1E+01 |
| Cells_Granularity_1_AGP | 5.7E−06 | 1.8E−05 | −7.1E+00 |
| Cells_Granularity_2_Lipid | 7.1E−13 | 6.8E−11 | 2.4E+01 |
| Cells_Intensity_IntegratedIntensity_Lipid | 7.1E−10 | 4.0E−09 | 1.5E+01 |
| Cells_Intensity_IntegratedIntensityEdge_Lipid | 1.1E−11 | 2.1E−10 | 2.0E+01 |
| Cells_Intensity_IntegratedIntensityEdge_DNA | 7.9E−06 | 2.4E−05 | −6.9E+00 |
| Cells_Intensity_LowerQuartileIntensity_Lipid | 3.2E−04 | 7.1E−04 | 4.7E+00 |
| Cells_Intensity_LowerQuartileIntensity_DNA | 3.0E−07 | 1.2E−06 | −9.1E+00 |
| Cells_Intensity_MADIntensity_Lipid | 2.8E−09 | 1.4E−08 | 1.3E+01 |
| Cells_Intensity_MADIntensity_DNA | 1.3E−05 | 3.9E−05 | −6.5E+00 |
| Cells_Intensity_MassDisplacement_Lipid | 1.8E−08 | 7.8E−08 | 1.1E+01 |
| Cells_Intensity_MassDisplacement_DNA | 5.3E−05 | 1.3E−04 | 5.7E+00 |
| Cells_Intensity_MaxIntensity_Lipid | 5.1E−10 | 3.0E−09 | 1.5E+01 |
| Cells_Intensity_MaxIntensityEdge_Lipid | 3.3E−11 | 4.6E−10 | 1.8E+01 |
| Cells_Intensity_MeanIntensity_Lipid | 1.2E−09 | 6.6E−09 | 1.4E+01 |
| Cells_Intensity_MeanIntensity_DNA | 1.2E−05 | 3.5E−05 | −6.6E+00 |
| Cells_Intensity_MeanIntensityEdge_Lipid | 1.6E−11 | 2.6E−10 | 1.9E+01 |
| Cells_Intensity_MeanIntensityEdge_DNA | 9.6E−07 | 3.3E−06 | −8.2E+00 |
| Cells_Intensity_MedianIntensity_Lipid | 4.8E−06 | 1.5E−05 | 7.2E+00 |
| Cells_Intensity_MedianIntensity_DNA | 5.9E−07 | 2.1E−06 | −8.6E+00 |
| Cells_Intensity_MinIntensity_DNA | 8.7E−09 | 3.9E−08 | −1.2E+01 |
| Cells_Intensity_MinIntensityEdge_DNA | 2.8E−08 | 1.2E−07 | −1.1E+01 |
| Cells_Intensity_StdIntensity_Lipid | 1.3E−11 | 2.3E−10 | 2.0E+01 |
| Cells_Intensity_StdIntensityEdge_Lipid | 1.7E−12 | 1.1E−10 | 2.3E+01 |
| Cells_Intensity_UpperQuartileIntensity_Lipid | 6.3E−09 | 2.9E−08 | 1.2E+01 |
| Cells_Intensity_UpperQuartileIntensity_DNA | 8.9E−06 | 2.7E−05 | −6.8E+00 |
| Nuclei_Texture_InfoMeas1_Mito_5_02 | 0.0017 | 0.0178 | 1.2208 |
| Nuclei_Texture_InverseDifferenceMoment_AGP_20_01 | 0.0107 | 0.0311 | 1.0068 |
| Cells_Mean_LargeLipidObjects_AreaShape_Area | 1.1E−11 | 2.1E−10 | 2.0E+01 |
| Cells_Mean_LargeLipidObjects_AreaShape_Center_X | 1.9E−12 | 1.1E−10 | 2.3E+01 |
| Cells_Mean_LargeLipidObjects_AreaShape_Center_Y | 1.1E−11 | 2.1E−10 | 2.0E+01 |
| Cells_Mean_LargeLipidObjects_AreaShape_Center_Z | 6.1E−12 | 1.7E−10 | 2.1E+01 |
| Cells_Mean_LargeLipidObjects_AreaShape_Compactness | 1.0E−11 | 2.1E−10 | 2.0E+01 |
| Cells_Mean_LargeLipidObjects_AreaShape_Eccentricity | 5.1E−12 | 1.7E−10 | 2.1E+01 |
| Cells_Mean_LargeLipidObjects_AreaShape_EulerNumber | 6.1E−12 | 1.7E−10 | 2.1E+01 |
| Cells_Mean_LargeLipidObjects_AreaShape_Extent | 2.5E−12 | 1.3E−10 | 2.2E+01 |
| Cells_Mean_LargeLipidObjects_AreaShape_FormFactor | 7.3E−13 | 6.8E−11 | 2.4E+01 |
| Cells_Mean_LargeLipidObjects_AreaShape_MajorAxisLength | 9.2E−12 | 2.1E−10 | 2.0E+01 |
| Cells_Mean_LargeLipidObjects_AreaShape_MaxFeretDiameter | 9.9E−12 | 2.1E−10 | 2.0E+01 |
| Cells_Mean_LargeLipidObjects_AreaShape_MaximumRadius | 3.0E−12 | 1.4E−10 | 2.2E+01 |
| Cells_Mean_LargeLipidObjects_AreaShape_MeanRadius | 2.8E−12 | 1.3E−10 | 2.2E+01 |
| Cells_Mean_LargeLipidObjects_AreaShape_MedianRadius | 2.8E−12 | 1.3E−10 | 2.2E+01 |
| Cells_Mean_LargeLipidObjects_AreaShape_MinFeretDiameter | 1.4E−11 | 2.4E−10 | 2.0E+01 |
| Cells_Mean_LargeLipidObjects_AreaShape_MinorAxisLength | 1.2E−11 | 2.1E−10 | 2.0E+01 |
| Cells_Mean_LargeLipidObjects_AreaShape_Perimeter | 2.9E−11 | 4.2E−10 | 1.9E+01 |
| Cells_Mean_LargeLipidObjects_AreaShape_Solidity | 3.2E−12 | 1.4E−10 | 2.2E+01 |
| Cells_Mean_LargeLipidObjects_Correlation_Correlation_Lipid_AGP | 5.5E−12 | 1.7E−10 | −2.1E+01 |
| Cells_Mean_LargeLipidObjects_Correlation_Correlation_DNA_AGP | 1.5E−10 | 1.0E−09 | 1.6E+01 |
| Cells_Mean_LargeLipidObjects_Correlation_Correlation_DNA_Lipid | 1.7E−15 | 9.5E−13 | −3.8E+01 |
| Cells_Mean_LargeLipidObjects_Correlation_Correlation_DNA_Mito | 4.2E−12 | 1.6E−10 | 2.1E+01 |
| Cells_Mean_LargeLipidObjects_Correlation_Correlation_Mito_AGP | 2.8E−10 | 1.8E−09 | 1.6E+01 |
| Cells_Mean_LargeLipidObjects_Correlation_Correlation_Mito_Lipid | 1.7E−15 | 9.5E−13 | −3.8E+01 |
| Cells_Mean_LargeLipidObjects_Correlation_K_AGP_Lipid | 3.0E−10 | 1.9E−09 | 1.6E+01 |
| Cells_Mean_LargeLipidObjects_Correlation_K_AGP_DNA | 2.8E−07 | 1.1E−06 | 9.1E+00 |
| Cells_Mean_LargeLipidObjects_Correlation_K_AGP_Mito | 2.2E−08 | 9.5E−08 | 1.1E+01 |
| Cells_Mean_LargeLipidObjects_Correlation_K_DNA_AGP | 3.1E−09 | 1.5E−08 | 1.3E+01 |
| Cells_Mean_LargeLipidObjects_Correlation_K_DNA_Lipid | 7.4E−12 | 1.9E−10 | 2.1E+01 |
| Cells_Mean_LargeLipidObjects_Correlation_K_DNA_Mito | 4.8E−09 | 2.3E−08 | 1.3E+01 |
| Cells_Mean_LargeLipidObjects_Correlation_K_Mito_AGP | 6.6E−08 | 2.7E−07 | 1.0E+01 |
| Cells_Mean_LargeLipidObjects_Correlation_K_Mito_Lipid | 6.0E−13 | 6.8E−11 | 2.5E+01 |
| Cells_Mean_LargeLipidObjects_Correlation_K_Mito_DNA | 1.9E−07 | 7.6E−07 | 9.5E+00 |
| Cells_Mean_LargeLipidObjects_Correlation_Overlap_Lipid_AGP | 5.2E−12 | 1.7E−10 | 2.1E+01 |
| Cells_Mean_LargeLipidObjects_Correlation_Overlap_DNA_AGP | 5.4E−12 | 1.7E−10 | 2.1E+01 |
| Cells_Mean_LargeLipidObjects_Correlation_Overlap_DNA_Lipid | 8.7E−12 | 2.1E−10 | 2.0E+01 |
| Cells_Mean_LargeLipidObjects_Correlation_Overlap_DNA_Mito | 5.9E−12 | 1.7E−10 | 2.1E+01 |
| Cells_Mean_LargeLipidObjects_Correlation_Overlap_Mito_AGP | 5.7E−12 | 1.7E−10 | 2.1E+01 |
| Cells_Mean_LargeLipidObjects_Correlation_Overlap_Mito_Lipid | 8.4E−12 | 2.1E−10 | 2.0E+01 |
| Cells_Mean_LargeLipidObjects_Granularity_13_Lipid | 7.3E−07 | 2.6E−06 | 8.4E+00 |
| Cells_Mean_LargeLipidObjects_Granularity_2_Lipid | 1.7E−14 | 5.9E−12 | 3.2E+01 |
| Cells_Mean_LargeLipidObjects_Intensity_IntegratedIntensity_Lipid | 3.9E−12 | 1.5E−10 | 2.2E+01 |
| Cells_Mean_LargeLipidObjects_Intensity_IntegratedIntensityEdge_Lipid | 1.1E−11 | 2.1E−10 | 2.0E+01 |
| Cells Mean_LargeLipidObjects_Intensity_LowerQuartileIntensity_Lipid | 7.9E−12 | 2.0E−10 | 2.0E+01 |
| Cells_Mean_LargeLipidObjects_Intensity_MADIntensity_Lipid | 6.7E−13 | 6.8E−11 | 2.5E+01 |
| Cells_Mean_LargeLipidObjects_Intensity_MassDisplacement_Lipid | 1.5E−10 | 1.0E−09 | 1.6E+01 |
| Cells_Mean_LargeLipidObjects_Intensity_MaxIntensity_Lipid | 4.8E−12 | 1.7E−10 | 2.1E+01 |
| Cells_Mean_LargeLipidObjects_Intensity_MaxIntensityEdge_Lipid | 5.6E−12 | 1.7E−10 | 2.1E+01 |
| Cells_Mean_LargeLipidObjects_Intensity_MeanIntensity_Lipid | 3.9E−12 | 1.5E−10 | 2.2E+01 |
| Cells_Mean_LargeLipidObjects_Intensity_MeanIntensityEdge_Lipid | 7.3E−12 | 1.9E−10 | 2.1E+01 |
| Cells_Mean_LargeLipidObjects_Intensity_MedianIntensity_Lipid | 5.0E−12 | 1.7E−10 | 2.1E+01 |
| Cells_Mean_LargeLipidObjects_Intensity_MinIntensity_Lipid | 1.5E−11 | 2.5E−10 | 1.9E+01 |
| Cells_Mean_LargeLipidObjects_Intensity_MinIntensityEdge_Lipid | 1.5E−11 | 2.5E−10 | 1.9E+01 |
| Cells_Mean_LargeLipidObjects_Intensity_StdIntensity_Lipid | 2.7E−12 | 1.3E−10 | 2.2E+01 |
| Cells_Mean_LargeLipidObjects_Intensity_StdIntensityEdge_Lipid | 3.8E−12 | 1.5E−10 | 2.2E+01 |
| Cells_Mean_LargeLipidObjects_Intensity_UpperQuartileIntensity_Lipid | 3.0E−12 | 1.4E−10 | 2.2E+01 |
| Cells_Mean_LargeLipidObjects_Location_Center_X | 1.9E−12 | 1.1E−10 | 2.3E+01 |
| Cells_Mean_LargeLipidObjects_Location_Center_Y | 1.1E−11 | 2.1E−10 | 2.0E+01 |
| Cells_Mean_LargeLipidObjects_Location_CenterMassIntensity_X_Lipid | 1.9E−12 | 1.1E−10 | 2.3E+01 |
| Cells_Mean_LargeLipidObjects_Location_CenterMassIntensity_Y_Lipid | 1.1E−11 | 2.1E−10 | 2.0E+01 |
| Cells_Mean_LargeLipidObjects_Location_MaxIntensity_X_Lipid | 1.9E−12 | 1.1E−10 | 2.3E+01 |
| Cells_Mean_LargeLipidObjects_Location_MaxIntensity_Y_Lipid | 1.1E−11 | 2.1E−10 | 2.0E+01 |
| Cells_Mean_LargeLipidObjects_Number_Object_Number | 5.4E−11 | 6.6E−10 | 1.8E+01 |
| Cells_Neighbors_FirstClosestObjectNumber_10 | 1.7E−04 | 3.8E−04 | −5.1E+00 |
| Cells_Neighbors_FirstClosestObjectNumber_Adjacent | 1.7E−04 | 3.8E−04 | −5.1E+00 |
| Cells_Neighbors_NumberOfNeighbors_10 | 4.4E−04 | 9.5E−04 | −4.6E+00 |
| Cells_Neighbors_SecondClosestDistance_10 | 3.9E−04 | 8.4E−04 | 4.6E+00 |
| Cells_Neighbors_SecondClosestDistance_Adjacent | 3.9E−04 | 8.4E−04 | 4.6E+00 |
| Cells_Neighbors_SecondClosestObjectNumber_10 | 1.5E−04 | 3.4E−04 | −5.1E+00 |
| Cells_Neighbors_SecondClosestObjectNumber_Adjacent | 1.5E−04 | 3.4E−04 | −5.1E+00 |
| Cells_Number_Object_Number | 1.6E−04 | 3.7E−04 | −5.1E+00 |
| Cells_Parent_Nuclei | 1.7E−04 | 3.8E−04 | −5.1E+00 |
| Cells_RadialDistribution_FracAtD_Lipid_2of4 | 1.0E−11 | 2.1E−10 | −2.0E+01 |
| Cells_RadialDistribution_MeanFrac_Lipid_2of4 | 4.1E−11 | 5.5E−10 | −1.8E+01 |
| Cells_RadialDistribution_RadialCV_Lipid_3of4 | 1.6E−10 | 1.1E−09 | 1.6E+01 |
| Cells_RadialDistribution_RadialCV_DNA_4of4 | 3.6E−05 | 9.2E−05 | 5.9E+00 |
| Cells_Texture_AngularSecondMoment_Lipid_20_01 | 4.9E−07 | 1.8E−06 | −8.7E+00 |
| Cells_Texture_AngularSecondMoment_DNA_10_03 | 3.6E−05 | 9.1E−05 | 5.9E+00 |
| Cells_Texture_Contrast_Lipid_20_01 | 6.9E−11 | 7.5E−10 | 1.7E+01 |
| Cells_Texture_Correlation_Lipid_10_02 | 1.9E−08 | 8.1E−08 | −1.1E+01 |
| Cells_Texture_Correlation_DNA_20_03 | 8.5E−06 | 2.6E−05 | 6.8E+00 |
| Cells_Texture_Correlation_Mito_20_00 | 1.1E−04 | 2.6E−04 | 5.3E+00 |
| Cells_Texture_DifferenceEntropy_Lipid_10_00 | 4.9E−11 | 6.2E−10 | 1.8E+01 |
| Cells_Texture_DifferenceEntropy_DNA_10_02 | 3.6E−06 | 1.2E−05 | −7.3E+00 |
| Cells_Texture_DifferenceVariance_Lipid_5_02 | 8.6E−07 | 3.0E−06 | −8.3E+00 |
| Cells_Texture_Entropy_Lipid_10_02 | 2.5E−09 | 1.2E−08 | 1.3E+01 |
| Cells_Texture_Entropy_DNA_10_02 | 1.8E−06 | 6.1E−06 | −7.8E+00 |
| Cells_Texture_InfoMeas1_Lipid_5_00 | 3.9E−12 | 1.5E−10 | −2.2E+01 |
| Cells_Texture_InfoMeas2_DNA_20_00 | 3.0E−05 | 7.8E−05 | −6.1E+00 |
| Cells_Texture_InverseDifferenceMoment_Lipid_20_01 | 6.1E−09 | 2.8E−08 | −1.2E+01 |
| Cells_Texture_InverseDifferenceMoment_DNA_20_02 | 2.5E−07 | 1.0E−06 | 9.2E+00 |
| Cells_Texture_SumAverage_Lipid_20_01 | 8.8E−10 | 4.8E−09 | 1.4E+01 |
| Cells_Texture_SumAverage_DNA_20_02 | 9.7E−06 | 2.9E−05 | −6.7E+00 |
| Cells_Texture_SumEntropy_Lipid_20_01 | 1.5E−10 | 1.1E−09 | 1.6E+01 |
| Cells_Texture_SumEntropy_DNA_10_02 | 5.2E−06 | 1.6E−05 | −7.1E+00 |
| Cells_Texture_SumVariance_Lipid_10_00 | 1.0E−10 | 8.9E−10 | 1.7E+01 |
| Cells_Texture_Variance_Lipid_20_01 | 8.7E−11 | 8.1E−10 | 1.7E+01 |
| Cytoplasm_AreaShape_Zernike_7_1 | 4.2E−05 | 1.1E−04 | −5.8E+00 |
| Cytoplasm_Correlation_Correlation_Lipid_AGP | 2.3E−15 | 9.8E−13 | −3.7E+01 |
| Cytoplasm_Correlation_Correlation_DNA_Lipid | 9.5E−14 | 2.3E−11 | −2.8E+01 |
| Cytoplasm_Correlation_Correlation_Mito_Lipid | 1.4E−12 | 9.9E−11 | −2.3E+01 |
| Cytoplasm_Correlation_K_AGP_Lipid | 2.8E−09 | 1.4E−08 | 1.3E+01 |
| Cytoplasm_Correlation_K_Lipid_AGP | 3.8E−10 | 2.3E−09 | −1.5E+01 |
| Cytoplasm_Correlation_K_Lipid_DNA | 1.5E−10 | 1.0E−09 | −1.6E+01 |
| Cytoplasm_Correlation_K_Lipid_Mito | 2.2E−08 | 9.5E−08 | −1.1 E+01 |
| Cytoplasm_Correlation_K_DNA_Lipid | 5.6E−13 | 6.8E−11 | 2.5E+01 |
| Cytoplasm_Correlation_K_DNA_Mito | 1.9E−05 | 5.3E−05 | 6.3E+00 |
| Cytoplasm_Correlation_K_Mito_Lipid | 5.1E−09 | 2.4E−08 | 1.3E+01 |
| Cytoplasm_Correlation_K_Mito_DNA | 1.3E−05 | 3.7E−05 | −6.6E+00 |
| Cytoplasm_Correlation_Overlap_Lipid_AGP | 7.2E−07 | 2.6E−06 | −8.5E+00 |
| Cytoplasm_Correlation_Overlap_DNA_Lipid | 5.7E−06 | 1.8E−05 | −7.1E+00 |
| Cytoplasm_Correlation_Overlap_Mito_Lipid | 2.6E−07 | 1.0E−06 | −9.2E+00 |
| Cytoplasm_Granularity_1_AGP | 1.1E−05 | 3.2E−05 | −6.7E+00 |
| Cytoplasm_Granularity_2_Lipid | 1.4E−15 | 9.5E−13 | 3.8E+01 |
| Cytoplasm_Intensity_IntegratedIntensity_Lipid | 3.3E−10 | 2.0E−09 | 1.6E+01 |
| Cytoplasm_Intensity_IntegratedIntensity_DNA | 2.6E−04 | 5.8E−04 | −4.8E+00 |
| Cytoplasm_Intensity_IntegratedIntensityEdge_Lipid | 3.1E−11 | 4.4E−10 | 1.8E+01 |
| Cytoplasm_Intensity_IntegratedIntensityEdge_DNA | 2.8E−07 | 1.1E−06 | −9.2E+00 |
| Cytoplasm_Intensity_LowerQuartileIntensity_DNA | 2.7E−07 | 1.1E−06 | −9.2E+00 |
| Cytoplasm_Intensity_MADIntensity_Lipid | 4.0E−10 | 2.4E−09 | 1.5E+01 |
| Cytoplasm_Intensity_MADIntensity_DNA | 4.7E−05 | 1.2E−04 | −5.8E+00 |
| Cytoplasm_Intensity_MassDisplacement_Lipid | 2.7E−09 | 1.3E−08 | 1.3E+01 |
| Cytoplasm_Intensity_MaxIntensity_Lipid | 5.0E−10 | 2.9E−09 | 1.5E+01 |
| Cytoplasm_Intensity_MaxIntensity_DNA | 7.5E−05 | 1.8E−04 | −5.5E+00 |
| Cytoplasm_Intensity_MaxIntensityEdge_Lipid | 4.8E−11 | 6.2E−10 | 1.8E+01 |
| Cytoplasm_Intensity_MaxIntensityEdge_DNA | 4.3E−05 | 1.1E−04 | −5.8E+00 |
| Cytoplasm_Intensity_MeanIntensity_Lipid | 3.6E−10 | 2.2E−09 | 1.5E+01 |
| Cytoplasm_Intensity_MeanIntensity_DNA | 2.7E−07 | 1.0E−06 | −9.2E+00 |
| Cytoplasm_Intensity_MeanIntensityEdge_Lipid | 5.4E−11 | 6.6E−10 | 1.8E+01 |
| Cytoplasm_Intensity_MeanIntensityEdge_DNA | 1.4E−07 | 5.6E−07 | −9.7E+00 |
| Cytoplasm_Intensity_MedianIntensity_Lipid | 3.7E−06 | 1.2E−05 | 7.3E+00 |
| Cytoplasm_Intensity_MedianIntensity_DNA | 5.9E−07 | 2.1E−06 | −8.6E+00 |
| Cytoplasm_Intensity_MinIntensity_DNA | 8.7E−09 | 3.9E−08 | −1.2E+01 |
| Cytoplasm_Intensity_MinIntensityEdge_DNA | 2.8E−08 | 1.2E−07 | −1.1E+01 |
| Cytoplasm_Intensity_StdIntensity_Lipid | 9.3E−12 | 2.1E−10 | 2.0E+01 |
| Cytoplasm_Intensity_StdIntensity_DNA | 8.0E−06 | 2.4E−05 | −6.8E+00 |
| Cytoplasm_Intensity_StdIntensityEdge_Lipid | 2.4E−12 | 1.3E−10 | 2.2E+01 |
| Cytoplasm_Intensity_StdIntensityEdge_DNA | 4.8E−05 | 1.2E−04 | −5.8E+00 |
| Cytoplasm_Intensity_UpperQuartileIntensity_Lipid | 5.2E−10 | 3.0E−09 | 1.5E+01 |
| Cytoplasm_Intensity_UpperQuartileIntensity_DNA | 8.3E−07 | 2.9E−06 | −8.3E+00 |
| Cytoplasm_Number_Object_Number | 1.6E−04 | 3.7E−04 | −5.1E+00 |
| Cytoplasm_Parent_Cells | 1.7E−04 | 3.8E−04 | −5.1E+00 |
| Cytoplasm_Parent_Nuclei | 1.7E−04 | 3.8E−04 | −5.1E+00 |
| Cytoplasm_RadialDistribution_FracAtD_AGP_1of4 | 1.7E−05 | 4.8E−05 | 6.4E+00 |
| Cytoplasm_RadialDistribution_MeanFrac_AGP_1of4 | 1.9E−05 | 5.3E−05 | 6.3E+00 |
| Cytoplasm_RadialDistribution_MeanFrac_Lipid_3of4 | 1.9E−07 | 7.7E−07 | −9.4E+00 |
| Cytoplasm_RadialDistribution_RadialCV_Lipid_2of4 | 8.4E−11 | 8.0E−10 | 1.7E+01 |
| Cytoplasm_Texture_AngularSecondMoment_Lipid_20_01 | 2.2E−07 | 9.0E−07 | −9.3E+00 |
| Cytoplasm_Texture_AngularSecondMoment_DNA_20_02 | 1.2E−04 | 2.8E−04 | 5.3E+00 |
| Cytoplasm_Texture_Contrast_Lipid_10_03 | 5.7E−11 | 6.7E−10 | 1.8E+01 |
| Cytoplasm_Texture_Contrast_DNA_5_02 | 1.3E−06 | 4.4E−06 | −8.0E+00 |
| Cytoplasm_Texture_Correlation_Lipid_5_02 | 7.5E−13 | 6.8E−11 | 2.4E+01 |
| Cytoplasm_Texture_DifferenceEntropy_Lipid_10_01 | 9.7E−12 | 2.1E−10 | 2.0E+01 |
| Cytoplasm_Texture_DifferenceEntropy_DNA_10_02 | 1.7E−06 | 5.7E−06 | −7.8E+00 |
| Cytoplasm_Texture_DifferenceVariance_Lipid_20_00 | 3.0E−07 | 1.1E−06 | −9.1E+00 |
| Cytoplasm_Texture_DifferenceVariance_DNA_20_02 | 1.8E−05 | 5.0E−05 | 6.4E+00 |
| Cytoplasm_Texture_Entropy_Lipid_10_02 | 5.5E−10 | 3.2E−09 | 1.5E+01 |
| Cytoplasm_Texture_Entropy_DNA_10_03 | 1.5E−05 | 4.3E−05 | −6.5E+00 |
| Cytoplasm_Texture_InfoMeas1_Lipid_5_00 | 2.8E−13 | 5.4E−11 | −2.6E+01 |
| Cytoplasm_Texture_InfoMeas2_DNA_20_00 | 6.1E−05 | 1.5E−04 | −5.6E+00 |
| Cytoplasm_Texture_InverseDifferenceMoment_Lipid_20_02 | 1.5E−09 | 7.9E−09 | −1.4E+01 |
| Cytoplasm_Texture_InverseDifferenceMoment_DNA_20_02 | 5.7E−06 | 1.8E−05 | 7.1E+00 |
| Cytoplasm_Texture_SumAverage_Lipid_10_02 | 2.7E−10 | 1.8E−09 | 1.6E+01 |
| Cytoplasm_Texture_SumAverage_DNA_10_01 | 2.5E−07 | 9.8E−07 | −9.2E+00 |
| Cytoplasm_Texture_SumEntropy_Lipid_10_01 | 4.1E−11 | 5.5E−10 | 1.8E+01 |
| Cytoplasm_Texture_SumEntropy_DNA_10_03 | 1.8E−05 | 5.1E−05 | −6.3E+00 |
| Cytoplasm_Texture_SumVariance_Lipid_10_00 | 6.8E−11 | 7.5E−10 | 1.7E+01 |
| Cytoplasm_Texture_SumVariance_DNA_10_00 | 2.7E−05 | 7.2E−05 | −6.1E+00 |
| Cytoplasm_Texture_Variance_Lipid_10_03 | 7.4E−11 | 7.7E−10 | 1.7E+01 |
| Cytoplasm_Texture_Variance_DNA_5_00 | 1.4E−05 | 4.1E−05 | −6.5E+00 |
| Nuclei_AreaShape_Zernike_3_3 | 4.4E−05 | 1.1E−04 | 5.8E+00 |
| Nuclei_Correlation_Correlation_Lipid_AGP | 1.0E−11 | 2.1E−10 | −2.0E+01 |
| Nuclei_Correlation_Correlation_DNA_Lipid | 3.4E−11 | 4.7E−10 | −1.8E+01 |
| Nuclei_Correlation_Correlation_Mito_Lipid | 1.3E−06 | 4.3E−06 | 8.0E+00 |
| Nuclei_Correlation_K_Lipid_AGP | 4.6E−05 | 1.1E−04 | −5.8E+00 |
| Nuclei_Correlation_K_Lipid_DNA | 1.7E−09 | 9.2E−09 | −1.4E+01 |
| Nuclei_Correlation_K_DNA_Lipid | 7.0E−08 | 2.9E−07 | 1.0E+01 |
| Nuclei_Correlation_K_DNA_Mito | 1.0E−05 | 3.0E−05 | 6.7E+00 |
| Nuclei_Correlation_K_Mito_DNA | 1.1E−05 | 3.3E−05 | −6.6E+00 |
| Nuclei_Correlation_Overlap_Lipid_AGP | 7.8E−12 | 2.0E−10 | −2.0E+01 |
| Nuclei_Correlation_Overlap_DNA_Lipid | 5.5E−12 | 1.7E−10 | −2.1E+01 |
| Nuclei_Granularity_10_Lipid | 8.3E−05 | 2.0E−04 | 5.5E+00 |
| Nuclei_Granularity_1_Lipid | 2.9E−06 | 9.3E−06 | −7.5E+00 |
| Nuclei_Intensity_IntegratedIntensityEdge_Lipid | 9.3E−07 | 3.2E−06 | 8.3E+00 |
| Nuclei_Intensity_MassDisplacement_Lipid | 5.2E−14 | 1.5E−11 | 3.0E+01 |
| Nuclei_Intensity_MassDisplacement_DNA | 3.6E−05 | 9.3E−05 | 5.9E+00 |
| Nuclei_Intensity_MaxIntensity_Lipid | 7.1E−09 | 3.2E−08 | 1.2E+01 |
| Nuclei_Intensity_MaxIntensityEdge_Lipid | 3.7E−10 | 2.2E−09 | 1.5E+01 |
| Nuclei_Intensity_MeanIntensityEdge_Lipid | 1.3E−06 | 4.4E−06 | 8.0E+00 |
| Nuclei_Intensity_MinIntensity_DNA | 1.7E−05 | 4.9E−05 | −6.4E+00 |
| Nuclei_Intensity_MinIntensityEdge_DNA | 1.7E−05 | 4.9E−05 | −6.4E+00 |
| Nuclei_Intensity_StdIntensity_Lipid | 1.6E−07 | 6.5E−07 | 9.6E+00 |
| Nuclei_Intensity_StdIntensityEdge_Lipid | 2.2E−11 | 3.3E−10 | 1.9E+01 |
| Nuclei_Neighbors_FirstClosestObjectNumber_2 | 1.5E−04 | 3.6E−04 | −5.1E+00 |
| Nuclei_Neighbors_SecondClosestObjectNumber_2 | 1.8E−04 | 4.1E−04 | −5.0E+00 |
| Nuclei_Number_Object_Number | 1.6E−04 | 3.7E−04 | −5.1E+00 |
| Nuclei_RadialDistribution_FracAtD_Lipid_2of4 | 7.2E−10 | 4.0E−09 | −1.5E+01 |
| Nuclei_RadialDistribution_MeanFrac_Lipid_2of4 | 2.0E−09 | 1.0E−08 | −1.3E+01 |
| Nuclei_RadialDistribution_RadialCV_Lipid_4of4 | 1.8E−13 | 3.9E−11 | 2.7E+01 |
| Nuclei_RadialDistribution_RadialCV_DNA_3of4 | 8.3E−07 | 2.9E−06 | 8.3E+00 |
| Nuclei_Texture_Contrast_Lipid_10_01 | 9.1E−10 | 5.0E−09 | 1.4E+01 |
| Nuclei_Texture_Correlation_Lipid_5_03 | 3.7E−12 | 1.5E−10 | 2.2E+01 |
| Nuclei_Texture_InfoMeas1_Lipid_10_03 | 1.2E−04 | 2.7E−04 | −5.3E+00 |
| Nuclei_Texture_SumAverage_Lipid_10_01 | 3.9E−04 | 8.4E−04 | 4.6E+00 |
| Nuclei_Texture_SumEntropy_Lipid_10_01 | 1.7E−05 | 4.9E−05 | 6.4E+00 |
| Nuclei_Texture_SumVariance_Lipid_10_01 | 2.5E−10 | 1.6E−09 | 1.6E+01 |
| Nuclei_Texture_Variance_Lipid_10_01 | 4.2E−10 | 2.5E−09 | 1.5E+01 |

PHH metformin treatment

| | | | |
|---|---|---|---|
| Cells_AreaShape_Area | 3.9E−11 | 2.0E−11 | −1.8E+01 |
| Cells_AreaShape_Center_Y | 1.1E−03 | 1.1E−04 | 4.1E+00 |
| Cells_AreaShape_Compactness | 3.7E−14 | 5.2E−14 | −3.0E+01 |
| Cells_AreaShape_Eccentricity | 2.8E−09 | 9.0E−10 | −1.3E+01 |
| Cells_AreaShape_EulerNumber | 7.9E−11 | 3.8E−11 | 1.7E+01 |
| Cells_AreaShape_Extent | 7.7E−16 | 2.5E−15 | 4.0E+01 |
| Cells_AreaShape_FormFactor | 1.5E−16 | 7.6E−16 | 4.5E+01 |
| Cells AreaShape_MajorAxisLength | 3.1E−12 | 2.1E−12 | −2.2E+01 |
| Cells_AreaShape_MaxFeretDiameter | 1.0E−12 | 8.3E−13 | −2.4E+01 |
| Cells_AreaShape_MaximumRadius | 2.8E−06 | 4.6E−07 | −7.5E+00 |
| Cells_AreaShape_MinFeretDiameter | 1.3E−12 | 1.0E−12 | −2.3E+01 |
| Cells_AreaShape_MinorAxisLength | 1.2E−11 | 7.2E−12 | −2.0E+01 |
| Cells_AreaShape_Perimeter | 1.8E−15 | 4.8E−15 | −3.8E+01 |
| Cells_AreaShape_Solidity | 2.0E−17 | 1.9E−16 | 5.2E+01 |
| Cells_AreaShape_Zernike_0_0 | 3.2E−16 | 1.2E−15 | 4.3E+01 |
| Cells_Children_LargeLipidObjects_Count | 2.4E−07 | 4.8E−08 | −9.3E+00 |
| Cells_Correlation_Correlation_Lipid_AGP | 1.4E−07 | 2.9E−08 | 9.7E+00 |
| Cells_Correlation_Correlation_DNA_AGP | 1.0E−18 | 2.8E−17 | −6.4E+01 |
| Cells_Correlation_Correlation_DNA_Lipid | 2.9E−06 | 4.6E−07 | 7.5E+00 |
| Cells_Correlation_Correlation_DNA_Mito | 1.5E−14 | 2.5E−14 | 3.2E+01 |
| Cells_Correlation_Correlation_Mito_AGP | 3.3E−18 | 5.6E−17 | 5.9E+01 |
| Cells_Correlation_Correlation_Mito_Lipid | 1.1E−13 | 1.2E−13 | 2.8E+01 |
| Cells_Correlation_K_AGP_Lipid | 3.0E−03 | 2.7E−04 | 3.6E+00 |
| Cells_Correlation_K_AGP_DNA | 9.2E−05 | 1.1E−05 | 5.4E+00 |
| Cells_Correlation_K_AGP_Mito | 1.3E−06 | 2.2E−07 | 8.0E+00 |
| Cells_Correlation_K_Lipid_AGP | 1.1E−05 | 1.6E−06 | −6.6E+00 |
| Cells_Correlation_K_Lipid_DNA | 2.8E−06 | 4.5E−07 | −7.5E+00 |
| Cells_Correlation_K_Lipid_Mito | 8.5E−08 | 1.9E−08 | 1.0E+01 |
| Cells_Correlation_K_DNA_AGP | 2.0E−06 | 3.2E−07 | −7.8E+00 |
| Cells_Correlation_K_DNA_Lipid | 8.8E−06 | 1.2E−06 | 6.8E+00 |
| Cells_Correlation_K_DNA_Mito | 1.8E−07 | 3.8E−08 | 9.5E+00 |
| Cells_Correlation_K_Mito_AGP | 1.3E−06 | 2.3E−07 | −8.0E+00 |
| Cells_Correlation_K_Mito_Lipid | 2.2E−07 | 4.6E−08 | −9.3E+00 |
| Cells_Correlation_K_Mito_DNA | 3.3E−06 | 5.3E−07 | −7.4E+00 |
| Cells_Correlation_Overlap_DNA_AGP | 2.2E−15 | 5.6E−15 | −3.7E+01 |
| Cells_Correlation_Overlap_DNA_Lipid | 2.3E−09 | 7.6E−10 | −1.3E+01 |
| Cells_Correlation_Overlap_Mito_AGP | 3.2E−17 | 2.6E−16 | 5.0E+01 |
| Cells_Correlation_Overlap_Mito_Lipid | 3.4E−07 | 6.7E−08 | 9.0E+00 |
| Cells_Granularity_10_AGP | 1.2E−12 | 9.4E−13 | 2.3E+01 |
| Cells_Granularity_10_Lipid | 2.5E−10 | 1.1E−10 | 1.6E+01 |
| Cells_Granularity_10_Mito | 2.1E−13 | 2.2E−13 | 2.7E+01 |
| Cells_Granularity_1_AGP | 9.7E−19 | 2.8E−17 | −6.5E+01 |
| Cells_Granularity_4_Lipid | 1.1E−15 | 3.5E−15 | 3.9E+01 |
| Cells_Granularity_3_Mito | 3.8E−18 | 5.8E−17 | 5.9E+01 |
| Cells_Intensity_IntegratedIntensity_AGP | 4.1E−03 | 3.6E−04 | −3.4E+00 |
| Cells_Intensity_IntegratedIntensity_Lipid | 2.1E−09 | 7.1E−10 | −1.3E+01 |
| Cells_Intensity_IntegratedIntensity_DNA | 6.1E−10 | 2.3E−10 | −1.5E+01 |
| Cells_Intensity_IntegratedIntensity_Mito | 6.2E−03 | 5.4E−04 | −3.2E+00 |
| Cells_Intensity_IntegratedIntensityEdge_AGP | 1.1E−05 | 1.5E−06 | −6.7E+00 |
| Cells_Intensity_IntegratedIntensityEdge_Lipid | 1.0E−12 | 8.2E−13 | −2.4E+01 |
| Cells_Intensity_IntegratedIntensityEdge_DNA | 1.3E−12 | 9.8E−13 | −2.3E+01 |
| Cells_Intensity_IntegratedIntensityEdge_Mito | 4.5E−10 | 1.8E−10 | −1.5E+01 |
| Cells_Intensity_LowerQuartileIntensity_AGP | 2.0E−07 | 4.1E−08 | 9.4E+00 |
| Cells_Intensity_LowerQuartileIntensity_Lipid | 4.7E−07 | 8.8E−08 | −8.8E+00 |
| Cells_Intensity_LowerQuartileIntensity_DNA | 1.7E−08 | 4.5E−09 | −1.1E+01 |
| Cells_Intensity_MADIntensity_AGP | 4.0E−07 | 7.8E−08 | 8.9E+00 |
| Cells_Intensity_MADIntensity_Lipid | 3.3E−03 | 2.9E−04 | 3.5E+00 |
| Cells_Intensity_MADIntensity_DNA | 5.6E−08 | 1.3E−08 | 1.0E+01 |
| Cells_Intensity_MADIntensity_Mito | 5.2E−08 | 1.2E−08 | 1.0E+01 |
| Cells_Intensity_MassDisplacement_AGP | 4.0E−12 | 2.6E−12 | −2.2E+01 |
| Cells_Intensity_MassDisplacement_Lipid | 6.5E−04 | 6.6E−05 | 4.4E+00 |
| Cells_Intensity_MassDisplacement_DNA | 1.3E−11 | 8.0E−12 | −2.0E+01 |
| Cells_Intensity_MassDisplacement_Mito | 1.6E−10 | 7.0E−11 | 1.6E+01 |
| Cells_Intensity_MaxIntensity_AGP | 1.4E−07 | 3.0E−08 | −9.7E+00 |
| Cells_Intensity_MaxIntensity_Lipid | 7.0E−07 | 1.3E−07 | −8.5E+00 |
| Cells_Intensity_MaxIntensityEdge_Lipid | 2.5E−09 | 8.3E−10 | −1.3E+01 |
| Cells_Intensity_MeanIntensity_Lipid | 6.1E−06 | 9.0E−07 | −7.0E+00 |
| Cells_Intensity_MeanIntensity_DNA | 1.4E−06 | 2.4E−07 | −8.0E+00 |
| Cells_Intensity_MeanIntensity_Mito | 1.0E−02 | 8.9E−04 | 3.0E+00 |
| Cells_Intensity_MeanIntensityEdge_Lipid | 1.6E−10 | 7.1E−11 | −1.6E+01 |
| Cells_Intensity_MeanIntensityEdge_DNA | 5.0E−09 | 1.5E−09 | −1.3E+01 |
| Cells_Intensity_MeanIntensityEdge_Mito | 1.3E−05 | 1.8E−06 | −6.5E+00 |
| Cells_Intensity_MedianIntensity_AGP | 8.2E−08 | 1.8E−08 | 1.0E+01 |
| Cells_Intensity_MedianIntensity_Lipid | 3.8E−04 | 4.0E−05 | −4.6E+00 |
| Cells_Intensity_MedianIntensity_DNA | 9.5E−04 | 9.3E−05 | −4.2E+00 |
| Cells_Intensity_MedianIntensity_Mito | 1.6E−03 | 1.5E−04 | 3.9E+00 |
| Cells_Intensity_MinIntensity_AGP | 1.8E−09 | 6.3E−10 | −1.4E+01 |
| Cells_Intensity_MinIntensity_Lipid | 1.0E−11 | 6.4E−12 | −2.0E+01 |
| Cells_Intensity_MinIntensity_DNA | 1.4E−14 | 2.3E−14 | −3.3E+01 |
| Cells_Intensity_MinIntensity_Mito | 1.6E−13 | 1.7E−13 | −2.7E+01 |
| Cells_Intensity_MinIntensityEdge_AGP | 1.1E−09 | 4.0E−10 | −1.4E+01 |
| Cells_Intensity_MinIntensityEdge_Lipid | 1.0E−11 | 6.3E−12 | −2.0E+01 |
| Cells_Intensity_MinIntensityEdge_DNA | 3.6E−15 | 8.2E−15 | −3.6E+01 |
| Cells_Intensity_MinIntensityEdge_Mito | 8.4E−14 | 1.0E−13 | −2.9E+01 |
| Cells_Intensity_StdIntensity_AGP | 2.5E−08 | 6.3E−09 | −1.1E+01 |
| Cells_Intensity_StdIntensity_Lipid | 8.6E−05 | 9.9E−06 | −5.5E+00 |
| Cells_Intensity_StdIntensity_DNA | 4.0E−04 | 4.2E−05 | −4.6E+00 |
| Cells_Intensity_StdIntensity_Mito | 1.4E−06 | 2.5E−07 | 8.0E+00 |
| Cells_Intensity_StdIntensityEdge_AGP | 7.0E−05 | 8.2E−06 | 5.6E+00 |
| Cells_Intensity_StdIntensityEdge_Lipid | 3.2E−08 | 8.0E−09 | −1.1E+01 |
| Cells_Intensity_StdIntensityEdge_DNA | 4.4E−07 | 8.4E−08 | 8.8E+00 |
| Cells_Intensity_StdIntensityEdge_Mito | 2.1E−08 | 5.3E−09 | 1.1E+01 |
| Cells_Intensity_UpperQuartileIntensity_AGP | 1.3E−04 | 1.4E−05 | 5.2E+00 |
| Cells_Intensity_UpperQuartileIntensity_Lipid | 7.4E−04 | 7.5E−05 | −4.3E+00 |
| Cells_Intensity_UpperQuartileIntensity_DNA | 1.5E−05 | 2.0E−06 | −6.5E+00 |
| Cells_Intensity_UpperQuartileIntensity_Mito | 1.5E−04 | 1.7E−05 | 5.1E+00 |
| Cells_Location_Center_Y | 9.3E−04 | 9.2E−05 | 4.2E+00 |
| Cells_Location_CenterMassIntensity_Y_AGP | 9.6E−04 | 9.4E−05 | 4.2E+00 |
| Cells_Location_CenterMassIntensity_Y_Lipid | 9.1E−04 | 9.0E−05 | 4.2E+00 |
| Cells_Location_CenterMassIntensity_Y_DNA | 1.0E−03 | 9.8E−05 | 4.1E+00 |
| Cells_Location_CenterMassIntensity_Y_Mito | 8.7E−04 | 8.6E−05 | 4.2E+00 |
| Cells_Location_MaxIntensity_Y_AGP | 1.2E−03 | 1.1E−04 | 4.1E+00 |
| Cells_Location_MaxIntensity_Y_Lipid | 1.0E−03 | 9.8E−05 | 4.1E+00 |
| Cells_Location_MaxIntensity_Y_DNA | 9.5E−04 | 9.3E−05 | 4.2E+00 |
| Cells_Location_MaxIntensity_Y_Mito | 1.2E−03 | 1.2E−04 | 4.0E+00 |
| Cells_Mean_LargeLipidObjects_AreaShape_Area | 3.8E−07 | 7.3E−08 | −8.9E+00 |
| Cells_Mean_LargeLipidObjects_AreaShape_Center_X | 2.8E−07 | 5.7E−08 | −9.1E+00 |
| Cells_Mean_LargeLipidObjects_AreaShape_Center_Y | 4.4E−07 | 8.5E−08 | −8.8E+00 |
| Cells_Mean_LargeLipidObjects_AreaShape_Center_Z | 3.7E−07 | 7.2E−08 | −8.9E+00 |
| Cells_Mean_LargeLipidObjects_AreaShape_Compactness | 4.3E−07 | 8.3E−08 | −8.8E+00 |
| Cells_Mean_LargeLipidObjects_AreaShape_Eccentricity | 3.7E−07 | 7.2E−08 | −8.9E+00 |
| Cells_Mean_LargeLipidObjects_AreaShape_EulerNumber | 3.7E−07 | 7.2E−08 | −8.9E+00 |
| Cells_Mean_LargeLipidObjects_AreaShape_Extent | 4.1E−07 | 7.8E−08 | −8.9E+00 |
| Cells_Mean_LargeLipidObjects_AreaShape_FormFactor | 1.2E−06 | 2.1E−07 | −8.1E+00 |
| Cells_Mean_LargeLipidObjects_AreaShape_MajorAxisLength | 4.1E−07 | 7.9E−08 | −8.9E+00 |
| Cells_Mean_LargeLipidObjects_AreaShape_MaxFeretDiameter | 3.9E−07 | 7.6E−08 | −8.9E+00 |
| Cells_Mean_LargeLipidObjects_AreaShape_MaximumRadius | 5.6E−07 | 1.0E−07 | −8.6E+00 |
| Cells_Mean_LargeLipidObjects_AreaShape_MeanRadius | 5.9E−07 | 1.1E−07 | −8.6E+00 |
| Cells_Mean_LargeLipidObjects_AreaShape_MedianRadius | 5.6E−07 | 1.0E−07 | −8.6E+00 |
| Cells_Mean_LargeLipidObjects_AreaShape_MinFeretDiameter | 3.0E−07 | 6.0E−08 | −9.1E+00 |
| Cells_Mean_LargeLipidObjects_AreaShape_MinorAxisLength | 3.2E−07 | 6.4E−08 | −9.0E+00 |
| Cells_Mean_LargeLipidObjects_AreaShape_Perimeter | 2.1E−07 | 4.2E−08 | −9.4E+00 |
| Cells_Mean_LargeLipidObjects_AreaShape_Solidity | 4.6E−07 | 8.8E−08 | −8.8E+00 |
| Cells_Mean_LargeLipidObjects_Correlation_Correlation_Lipid_AGP | 1.3E−07 | 2.9E−08 | 9.7E+00 |
| Cells_Mean_LargeLipidObjects_Correlation_Correlation_DNA_AGP | 6.0E−07 | 1.1E−07 | −8.6E+00 |
| Cells_Mean_LargeLipidObjects_Correlation_Correlation_DNA_Lipid | 8.4E−08 | 1.9E−08 | 1.0E+01 |
| Cells_Mean_LargeLipidObjects_Correlation_Correlation_DNA_Mito | 2.3E−07 | 4.7E−08 | −9.3E+00 |
| Cells_Mean_LargeLipidObjects_Correlation_Correlation_Mito_AGP | 1.2E−06 | 2.1E−07 | −8.1E+00 |
| Cells_Mean_LargeLipidObjects_Correlation_Correlation_Mito_Lipid | 2.8E−07 | 5.6E−08 | 9.1E+00 |
| Cells_Mean_LargeLipidObjects_Correlation_K_AGP_Lipid | 7.7E−09 | 2.2E−09 | −1.2E+01 |
| Cells_Mean_LargeLipidObjects_Correlation_K_AGP_DNA | 6.2E−09 | 1.8E−09 | −1.2E+01 |
| Cells_Mean_LargeLipidObjects_Correlation_K_AGP_Mito | 3.0E−08 | 7.4E−09 | −1.1E+01 |
| Cells_Mean_LargeLipidObjects_Correlation_K_DNA_Lipid | 8.7E−08 | 1.9E−08 | −1.0E+01 |
| Cells_Mean_LargeLipidObjects_Correlation_K_DNA_Mito | 3.7E−04 | 3.9E−05 | −4.7E+00 |
| Cells_Mean_LargeLipidObjects_Correlation_K_Mito_AGP | 7.4E−05 | 8.7E−06 | −5.5E+00 |
| Cells_Mean_LargeLipidObjects_Correlation_K_Mito_Lipid | 1.1E−07 | 2.3E−08 | −9.9E+00 |
| Cells_Mean_LargeLipidObjects_Correlation_K_Mito_DNA | 1.3E−07 | 2.8E−08 | −9.7E+00 |
| Cells_Mean_LargeLipidObjects_Correlation_Overlap_Lipid_AGP | 5.1E−07 | 9.5E−08 | −8.7E+00 |
| Cells_Mean_LargeLipidObjects_Correlation_Overlap_DNA_AGP | 3.9E−07 | 7.6E−08 | −8.9E+00 |
| Cells_Mean_LargeLipidObjects_Correlation_Overlap_DNA_Lipid | 5.2E−07 | 9.7E−08 | −8.7E+00 |
| Cells_Mean_LargeLipidObjects_Correlation_Overlap_DNA_Mito | 3.8E−07 | 7.3E−08 | −8.9E+00 |
| Cells_Mean_LargeLipidObjects_Correlation_Overlap_Mito_AGP | 4.7E−07 | 8.8E−08 | −8.8E+00 |
| Cells_Mean_LargeLipidObjects_Correlation_Overlap_Mito_Lipid | 6.2E−07 | 1.1E−07 | −8.6E+00 |
| Cells_Mean_LargeLipidObjects_Granularity_16_Lipid | 1.4E−04 | 1.5E−05 | −5.2E+00 |
| Cells_Mean_LargeLipidObjects_Granularity_1_Lipid | 2.0E−08 | 5.2E−09 | −1.1E+01 |
| Cells_Mean_LargeLipidObjects_Intensity_IntegratedIntensity_Lipid | 1.6E−07 | 3.3E−08 | −9.6E+00 |
| Cells_Mean_LargeLipidObjects_Intensity_IntegratedIntensityEdge_Lipid | 2.0E−07 | 4.2E−08 | −9.4E+00 |
| Cells_Mean_LargeLipidObjects_Intensity_LowerQuartileIntensity_Lipid | 2.2E−07 | 4.6E−08 | −9.3E+00 |
| Cells_Mean_LargeLipidObjects_Intensity_MADIntensity_Lipid | 5.7E−08 | 1.4E−08 | −1.0E+01 |
| Cells_Mean_LargeLipidObjects_Intensity_MassDisplacement_Lipid | 1.7E−06 | 2.8E−07 | −7.9E+00 |
| Cells_Mean_LargeLipidObjects_Intensity_MaxIntensity_Lipid | 1.6E−07 | 3.3E−08 | −9.6E+00 |
| Cells_Mean_LargeLipidObjects_Intensity_MaxIntensityEdge_Lipid | 4.9E−07 | 9.3E−08 | −8.7E+00 |
| Cells_Mean_LargeLipidObjects_Intensity_MeanIntensity_Lipid | 1.4E−07 | 2.9E−08 | −9.7E+00 |
| Cells_Mean_LargeLipidObjects_Intensity_MeanIntensityEdge_Lipid | 3.2E−07 | 6.3E−08 | −9.0E+00 |
| Cells_Mean_LargeLipidObjects_Intensity_MedianIntensity_Lipid | 1.2E−07 | 2.7E−08 | −9.8E+00 |
| Cells_Mean_LargeLipidObjects_Intensity_MinIntensity_Lipid | 7.6E−07 | 1.4E−07 | −8.4E+00 |
| Cells_Mean_LargeLipidObjects_Intensity_MinIntensityEdge_Lipid | 6.9E−07 | 1.3E−07 | −8.5E+00 |
| Cells_Mean_LargeLipidObjects_Intensity_StdIntensity_Lipid | 1.3E−07 | 2.8E−08 | −9.7E+00 |
| Cells_Mean_LargeLipidObjects_Intensity_StdIntensityEdge_Lipid | 5.3E−07 | 9.9E−08 | −8.7E+00 |
| Cells_Mean_LargeLipidObjects_Intensity_UpperQuartileIntensity_Lipid | 1.1E−07 | 2.4E−08 | −9.9E+00 |
| Cells_Mean_LargeLipidObjects_Location_Center_X | 2.8E−07 | 5.7E−08 | −9.1E+00 |
| Cells_Mean_LargeLipidObjects_Location_Center_Y | 4.4E−07 | 8.5E−08 | −8.8E+00 |
| Cells_Mean_LargeLipidObjects_Location_CenterMassIntensity_X_Lipid | 2.8E−07 | 5.7E−08 | −9.1E+00 |
| Cells_Mean_LargeLipidObjects_Location_CenterMassIntensity_Y_Lipid | 4.4E−07 | 8.5E−08 | −8.8E+00 |
| Cells_Mean_LargeLipidObjects_Location_MaxIntensity_X_Lipid | 2.8E−07 | 5.7E−08 | −9.1E+00 |
| Cells_Mean_LargeLipidObjects_Location_MaxIntensity_Y_Lipid | 4.5E−07 | 8.5E−08 | −8.8E+00 |
| Cells_Mean_LargeLipidObjects_Number_Object_Number | 8.8E−07 | 1.6E−07 | −8.3E+00 |
| Cells_Neighbors_AngleBetweenNeighbors_10 | 1.0E−12 | 8.3E−13 | −2.4E+01 |
| Cells_Neighbors_AngleBetweenNeighbors_Adjacent | 1.0E−12 | 8.3E−13 | −2.4E+01 |
| Cells_Neighbors_FirstClosestObjectNumber_10 | 6.4E−09 | 1.8E−09 | −1.2E+01 |
| Cells_Neighbors_FirstClosestObjectNumber_Adjacent | 6.4E−09 | 1.8E−09 | −1.2E+01 |
| Cells_Neighbors_NumberOfNeighbors_10 | 3.9E−16 | 1.4E−15 | −4.2E+01 |
| Cells_Neighbors_NumberOfNeighbors_Adjacent | 2.2E−16 | 9.4E−16 | −4.4E+01 |
| Cells_Neighbors_PercentTouching_10 | 1.8E−15 | 4.8E−15 | −3.8E+01 |
| Cells_Neighbors_PercentTouching_Adjacent | 3.8E−11 | 2.0E−11 | −1.8E+01 |
| Cells_Neighbors_SecondClosestDistance_10 | 1.7E−03 | 1.6E−04 | 3.9E+00 |
| Cells_Neighbors_SecondClosestDistance_Adjacent | 1.7E−03 | 1.6E−04 | 3.9E+00 |
| Cells_Neighbors_SecondClosestObjectNumber_10 | 5.6E−09 | 1.7E−09 | −1.2E+01 |
| Cells_Neighbors_SecondClosestObjectNumber_Adjacent | 5.6E−09 | 1.7E−09 | −1.2E+01 |
| Cells_Number_Object_Number | 6.0E−09 | 1.7E−09 | −1.2E+01 |
| Cells_Parent_Nuclei | 6.1E−09 | 1.8E−09 | −1.2E+01 |
| Cells_RadialDistribution_FracAtD_AGP_2of4 | 3.2E−12 | 2.2E−12 | −2.2E+01 |
| Cells_RadialDistribution_FracAtD_Lipid_4of4 | 5.3E−18 | 6.6E−17 | −5.7E+01 |
| Cells_RadialDistribution_FracAtD_DNA_1of4 | 3.4E−14 | 4.9E−14 | 3.0E+01 |
| Cells_RadialDistribution_FracAtD_Mito_1of4 | 7.4E−17 | 4.8E−16 | 4.7E+01 |
| Cells_RadialDistribution_MeanFrac_AGP_2of4 | 4.6E−16 | 1.6E−15 | −4.2E+01 |
| Cells_RadialDistribution_MeanFrac_Lipid_4of4 | 3.9E−16 | 1.4E−15 | −4.2E+01 |
| Cells_RadialDistribution_MeanFrac_DNA_3of4 | 6.3E−16 | 2.1E−15 | −4.1E+01 |
| Cells_RadialDistribution_MeanFrac_Mito_4of4 | 7.5E−16 | 2.5E−15 | −4.0E+01 |
| Cells_RadialDistribution_RadialCV_AGP_3of4 | 2.2E−16 | 9.4E−16 | −4.4E+01 |
| Cells_RadialDistribution_RadialCV_Lipid_4of4 | 2.2E−09 | 7.4E−10 | 1.3E+01 |
| Cells_RadialDistribution_RadialCV_DNA_4of4 | 2.4E−10 | 1.0E−10 | 1.6E+01 |
| Cells_RadialDistribution_RadialCV_Mito_4of4 | 2.3E−16 | 9.4E−16 | 4.4E+01 |
| Cells_Texture_AngularSecondMoment_AGP_5_01 | 8.5E−08 | 1.9E−08 | −1.0E+01 |
| Cells_Texture_AngularSecondMoment_DNA_20_00 | 9.4E−07 | 1.7E−07 | −8.3E+00 |
| Cells_Texture_AngularSecondMoment_Mito_20_02 | 6.7E−05 | 7.9E−06 | −5.6E+00 |
| Cells_Texture_Contrast_AGP_20_00 | 1.6E−09 | 5.4E−10 | −1.4E+01 |
| Cells_Texture_Contrast_Lipid_5_02 | 5.8E−05 | 7.0E−06 | −5.7E+00 |
| Cells_Texture_Contrast_DNA_20_03 | 2.1E−04 | 2.3E−05 | −4.9E+00 |
| Cells_Texture_Contrast_Mito_20_01 | 1.7E−06 | 2.8E−07 | 7.9E+00 |
| Cells_Texture_Correlation_AGP_5_00 | 1.3E−14 | 2.2E−14 | −3.3E+01 |
| Cells_Texture_Correlation_Lipid_20_01 | 6.7E−13 | 5.9E−13 | −2.5E+01 |
| Cells_Texture_Correlation_DNA_20_03 | 1.4E−18 | 3.0E−17 | −6.3E+01 |
| Cells_Texture_Correlation_Mito_20_01 | 4.4E−17 | 3.1E−16 | −4.9E+01 |
| Cells_Texture_DifferenceEntropy_AGP_20_00 | 1.5E−05 | 2.0E−06 | −6.5E+00 |
| Cells_Texture_DifferenceEntropy_Lipid_20_01 | 2.2E−03 | 2.0E−04 | −3.7E+00 |
| Cells_Texture_DifferenceEntropy_DNA_10_03 | 2.9E−07 | 5.8E−08 | 9.1E+00 |
| Cells_Texture_DifferenceEntropy_Mito_10_01 | 1.3E−05 | 1.7E−06 | 6.6E+00 |
| Cells_Texture_DifferenceVariance_Lipid_5_00 | 6.4E−04 | 6.5E−05 | 4.4E+00 |
| Cells_Texture_DifferenceVariance_DNA_10_03 | 4.6E−06 | 7.0E−07 | −7.2E+00 |
| Cells_Texture_DifferenceVariance_Mito_20_01 | 2.7E−04 | 2.9E−05 | −4.8E+00 |
| Cells_Texture_Entropy_AGP_5_00 | 1.7E−05 | 2.3E−06 | 6.4E+00 |
| Cells_Texture_Entropy_Lipid_20_01 | 7.0E−04 | 7.1E−05 | −4.3E+00 |
| Cells_Texture_Entropy_DNA_10_03 | 4.4E−08 | 1.1E−08 | 1.1E+01 |
| Cells_Texture_Entropy_Mito_10_01 | 6.7E−06 | 9.8E−07 | 7.0E+00 |
| Cells_Texture_InfoMeas1_AGP_5_00 | 1.3E−14 | 2.2E−14 | 3.3E+01 |
| Cells_Texture_InfoMeas1_Lipid_5_00 | 1.6E−05 | 2.2E−06 | −6.4E+00 |
| Cells_Texture_InfoMeas1_DNA_20_03 | 7.7E−14 | 9.5E−14 | −2.9E+01 |
| Cells_Texture_InfoMeas1_Mito_20_01 | 3.4E−13 | 3.3E−13 | −2.6E+01 |
| Cells_Texture_InverseDifferenceMoment_AGP_20_03 | 1.0E−08 | 2.8E−09 | −1.2E+01 |
| Cells_Texture_InverseDifferenceMoment_Lipid_20_00 | 1.2E−03 | 1.1E−04 | −4.1E+00 |
| Cells_Texture_InverseDifferenceMoment_DNA_10_03 | 2.2E−10 | 9.4E−11 | −1.6E+01 |
| Cells_Texture_InverseDifferenceMoment_Mito_20_01 | 9.7E−09 | 2.7E−09 | −1.2E+01 |
| Cells_Texture_SumAverage_Lipid_20_01 | 7.9E−07 | 1.4E−07 | −8.4E+00 |
| Cells_Texture_SumAverage_DNA_20_03 | 2.9E−07 | 5.7E−08 | −9.1E+00 |
| Cells_Texture_SumAverage_Mito_10_00 | 2.1E−03 | 2.0E−04 | 3.8E+00 |
| Cells_Texture_SumEntropy_AGP_20_00 | 1.5E−03 | 1.5E−04 | −3.9E+00 |
| Cells_Texture_SumEntropy_Lipid_20_01 | 8.8E−05 | 1.0E−05 | −5.4E+00 |
| Cells_Texture_SumEntropy_DNA_10_01 | 4.7E−06 | 7.2E−07 | 7.2E+00 |
| Cells_Texture_SumEntropy_Mito_5_00 | 2.4E−06 | 4.0E−07 | 7.6E+00 |
| Cells_Texture_SumVariance_AGP_5_01 | 1.3E−09 | 4.7E−10 | −1.4E+01 |
| Cells_Texture_SumVariance_Lipid_20_01 | 9.8E−06 | 1.4E−06 | −6.7E+00 |
| Cells_Texture_SumVariance_DNA_20_03 | 4.1E−05 | 5.2E−06 | −5.9E+00 |
| Cells_Texture_SumVariance_Mito_5_02 | 2.7E−06 | 4.4E−07 | 7.5E+00 |
| Cells_Texture_Variance_AGP_10_03 | 2.3E−09 | 7.6E−10 | −1.3E+01 |
| Cells_Texture_Variance_Lipid_20_01 | 3.2E−05 | 4.1E−06 | −6.0E+00 |
| Cells_Texture_Variance_DNA_20_03 | 1.1E−04 | 1.2E−05 | −5.3E+00 |
| Cells_Texture_Variance_Mito_10_01 | 5.3E−06 | 8.0E−07 | 7.1E+00 |
| Cytoplasm_AreaShape_Area | 2.0E−11 | 1.1E−11 | −1.9E+01 |
| Cytoplasm_AreaShape_Center_Y | 9.2E−04 | 9.1E−05 | 4.2E+00 |
| Cytoplasm_AreaShape_Eccentricity | 3.9E−06 | 6.1E−07 | −7.3E+00 |
| Cytoplasm_AreaShape_EulerNumber | 2.3E−12 | 1.6E−12 | 2.2E+01 |
| Cytoplasm_AreaShape_Extent | 5.4E−10 | 2.1E−10 | 1.5E+01 |
| Cytoplasm_AreaShape_FormFactor | 4.1E−14 | 5.7E−14 | 3.0E+01 |
| Cytoplasm_AreaShape_MajorAxisLength | 6.5E−12 | 4.2E−12 | −2.1E+01 |
| Cytoplasm_AreaShape_MaxFeretDiameter | 1.0E−12 | 8.3E−13 | −2.4E+01 |
| Cytoplasm_AreaShape_MaximumRadius | 7.2E−11 | 3.6E−11 | −1.7E+01 |
| Cytoplasm_AreaShape_MeanRadius | 3.2E−08 | 7.9E−09 | −1.1E+01 |
| Cytoplasm_AreaShape_MedianRadius | 2.0E−05 | 2.6E−06 | −6.3E+00 |
| Cytoplasm_AreaShape_MinFeretDiameter | 1.3E−12 | 9.6E−13 | −2.3E+01 |
| Cytoplasm_AreaShape_MinorAxisLength | 1.4E−11 | 8.4E−12 | −2.0E+01 |
| Cytoplasm_AreaShape_Perimeter | 3.5E−15 | 8.0E−15 | −3.6E+01 |
| Cytoplasm_AreaShape_Solidity | 6.9E−11 | 3.4E−11 | 1.7E+01 |
| Cytoplasm_AreaShape_Zernike_4_0 | 8.3E−17 | 5.2E−16 | 4.7E+01 |
| Cytoplasm_Correlation_Correlation_Lipid_AGP | 2.0E−13 | 2.1E−13 | 2.7E+01 |
| Cytoplasm_Correlation_Correlation_DNA_AGP | 2.7E−08 | 6.7E−09 | −1.1E+01 |
| Cytoplasm_Correlation_Correlation_DNA_Lipid | 4.3E−14 | 5.8E−14 | 3.0E+01 |
| Cytoplasm_Correlation_Correlation_DNA_Mito | 2.0E−07 | 4.0E−08 | 9.4E+00 |
| Cytoplasm_Correlation_Correlation_Mito_AGP | 4.2E−14 | 5.8E−14 | 3.0E+01 |
| Cytoplasm_Correlation_Correlation_Mito_Lipid | 3.3E−13 | 3.2E−13 | 2.6E+01 |
| Cytoplasm_Correlation_K_AGP_Lipid | 3.3E−08 | 8.1E−09 | −1.1E+01 |
| Cytoplasm_Correlation_K_AGP_DNA | 1.8E−07 | 3.7E−08 | −9.5E+00 |
| Cytoplasm_Correlation_K_Lipid_AGP | 4.3E−07 | 8.2E−08 | 8.8E+00 |
| Cytoplasm_Correlation_K_Lipid_DNA | 7.0E−07 | 1.3E−07 | 8.5E+00 |
| Cytoplasm_Correlation_K_Lipid_Mito | 1.5E−07 | 3.1E−08 | 9.6E+00 |
| Cytoplasm_Correlation_K_DNA_AGP | 1.6E−06 | 2.7E−07 | 7.9E+00 |
| Cytoplasm_Correlation_K_DNA_Lipid | 5.8E−07 | 1.1E−07 | −8.6E+00 |
| Cytoplasm_Correlation_K_DNA_Mito | 8.8E−05 | 1.0E−05 | 5.4E+00 |
| Cytoplasm_Correlation_K_Mito_Lipid | 2.6E−07 | 5.3E−08 | −9.2E+00 |
| Cytoplasm_Correlation_K_Mito_DNA | 1.0E−03 | 1.0E−04 | −4.1E+00 |
| Cytoplasm_Correlation_Overlap_Lipid_AGP | 1.6E−07 | 3.4E−08 | 9.6E+00 |
| Cytoplasm_Correlation_Overlap_DNA_AGP | 3.1E−08 | 7.8E−09 | −1.1E+01 |
| Cytoplasm_Correlation_Overlap_DNA_Lipid | 1.3E−08 | 3.4E−09 | 1.2E+01 |
| Cytoplasm_Correlation_Overlap_Mito_AGP | 3.4E−15 | 8.0E−15 | 3.6E+01 |
| Cytoplasm_Correlation_Overlap_Mito_Lipid | 4.5E−07 | 8.6E−08 | 8.8E+00 |
| Cytoplasm_Granularity_10_AGP | 2.2E−12 | 1.5E−12 | 2.2E+01 |
| Cytoplasm_Granularity_10_Lipid | 1.7E−10 | 7.6E−11 | 1.6E+01 |
| Cytoplasm_Granularity_10_Mito | 2.7E−13 | 2.7E−13 | 2.6E+01 |
| Cytoplasm_Granularity_1_AGP | 1.2E−18 | 2.8E−17 | −6.4E+01 |
| Cytoplasm_Granularity_4_Lipid | 1.7E−15 | 4.5E−15 | 3.8E+01 |
| Cytoplasm_Granularity_3_Mito | 4.1E−18 | 5.8E−17 | 5.8E+01 |
| Cytoplasm_Intensity_IntegratedIntensity_Lipid | 1.3E−09 | 4.7E−10 | −1.4E+01 |
| Cytoplasm_Intensity_IntegratedIntensity_DNA | 4.1E−09 | 1.2E−09 | −1.3E+01 |
| Cytoplasm_Intensity_IntegratedIntensity_Mito | 4.3E−04 | 4.5E−05 | −4.6E+00 |
| Cytoplasm_Intensity_IntegratedIntensityEdge_AGP | 3.6E−05 | 4.6E−06 | −5.9E+00 |
| Cytoplasm_Intensity_IntegratedIntensityEdge_Lipid | 2.9E−11 | 1.6E−11 | −1.9E+01 |
| Cytoplasm_Intensity_IntegratedIntensityEdge_DNA | 1.7E−12 | 1.2E−12 | −2.3E+01 |
| Cytoplasm_Intensity_IntegratedIntensityEdge_Mito | 3.3E−07 | 6.6E−08 | −9.0E+00 |
| Cytoplasm_Intensity_LowerQuartileIntensity_AGP | 4.8E−07 | 9.1E−08 | 8.7E+00 |
| Cytoplasm_Intensity_LowerQuartileIntensity_Lipid | 7.9E−08 | 1.8E−08 | −1.0E+01 |
| Cytoplasm_Intensity_LowerQuartileIntensity_DNA | 1.4E−09 | 4.8E−10 | −1.4E+01 |
| Cytoplasm_Intensity_LowerQuartileIntensity_Mito | 6.4E−03 | 5.6E−04 | −3.2E+00 |
| Cytoplasm_Intensity_MADIntensity_AGP | 3.5E−09 | 1.1E−09 | 1.3E+01 |
| Cytoplasm_Intensity_MADIntensity_Lipid | 1.3E−03 | 1.3E−04 | 4.0E+00 |
| Cytoplasm_Intensity_MADIntensity_DNA | 1.8E−07 | 3.7E−08 | 9.5E+00 |
| Cytoplasm_Intensity_MADIntensity_Mito | 2.7E−08 | 6.8E−09 | 1.1E+01 |
| Cytoplasm_Intensity_MassDisplacement_AGP | 5.8E−03 | 5.1E−04 | −3.2E+00 |
| Cytoplasm_Intensity_MassDisplacement_Lipid | 1.7E−04 | 1.9E−05 | 5.1E+00 |
| Cytoplasm_Intensity_MassDisplacement_Mito | 6.9E−11 | 3.4E−11 | 1.7E+01 |
| Cytoplasm_Intensity_MaxIntensity_AGP | 8.4E−03 | 7.2E−04 | 3.1E+00 |
| Cytoplasm_Intensity_MaxIntensity_Lipid | 5.3E−07 | 9.9E−08 | −8.7E+00 |
| Cytoplasm_Intensity_MaxIntensity_DNA | 2.3E−08 | 5.9E−09 | −1.1E+01 |
| Cytoplasm_Intensity_MaxIntensityEdge_Lipid | 1.7E−07 | 3.6E−08 | −9.5E+00 |
| Cytoplasm_Intensity_MaxIntensityEdge_DNA | 5.5E−09 | 1.6E−09 | −1.3E+01 |
| Cytoplasm_Intensity_MeanIntensity_AGP | 5.7E−07 | 1.1E−07 | 8.6E+00 |
| Cytoplasm_Intensity_MeanIntensity_Lipid | 3.0E−06 | 4.8E−07 | −7.5E+00 |
| Cytoplasm_Intensity_MeanIntensity_DNA | 1.5E−05 | 2.1E−06 | −6.4E+00 |
| Cytoplasm_Intensity_MeanIntensityEdge_Lipid | 1.4E−08 | 3.7E−09 | −1.2E+01 |
| Cytoplasm_Intensity_MeanIntensityEdge_DNA | 1.7E−08 | 4.5E−09 | −1.1E+01 |
| Cytoplasm_Intensity_MedianIntensity_AGP | 3.3E−08 | 8.0E−09 | 1.1E+01 |
| Cytoplasm_Intensity_MedianIntensity_Lipid | 5.9E−05 | 7.1E−06 | −5.7E+00 |
| Cytoplasm_Intensity_MedianIntensity_DNA | 2.6E−05 | 3.3E−06 | −6.1E+00 |
| Cytoplasm_Intensity_MinIntensity_AGP | 1.9E−09 | 6.4E−10 | −1.4E+01 |
| Cytoplasm_Intensity_MinIntensity_Lipid | 1.0E−11 | 6.4E−12 | −2.0E+01 |
| Cytoplasm_Intensity_MinIntensity_DNA | 1.4E−14 | 2.3E−14 | −3.3E+01 |
| Cytoplasm_Intensity_MinIntensity_Mito | 1.5E−13 | 1.6E−13 | −2.7E+01 |
| Cytoplasm_Intensity_MinIntensityEdge_AGP | 1.1E−09 | 3.9E−10 | −1.4E+01 |
| Cytoplasm_Intensity_MinIntensityEdge_Lipid | 1.0E−11 | 6.2E−12 | −2.0E+01 |
| Cytoplasm_Intensity_MinIntensityEdge_DNA | 3.6E−15 | 8.2E−15 | −3.6E+01 |
| Cytoplasm_Intensity_MinIntensityEdge_Mito | 8.9E−14 | 1.1E−13 | −2.8E+01 |
| Cytoplasm_Intensity_StdIntensity_AGP | 1.4E−05 | 1.9E−06 | 6.5E+00 |
| Cytoplasm_Intensity_StdIntensity_Lipid | 3.9E−05 | 4.9E−06 | −5.9E+00 |
| Cytoplasm_Intensity_StdIntensity_Mito | 8.7E−07 | 1.6E−07 | 8.3E+00 |
| Cytoplasm_Intensity_StdIntensityEdge_Lipid | 2.4E−05 | 3.1E−06 | −6.2E+00 |
| Cytoplasm_Intensity_StdIntensityEdge_DNA | 6.3E−03 | 5.5E−04 | −3.2E+00 |
| Cytoplasm_Intensity_StdIntensityEdge_Mito | 2.9E−08 | 7.1E−09 | 1.1E+01 |
| Cytoplasm_Intensity_UpperQuartileIntensity_AGP | 1.0E−07 | 2.2E−08 | 9.9E+00 |
| Cytoplasm_Intensity_UpperQuartileIntensity_Lipid | 9.1E−04 | 9.0E−05 | −4.2E+00 |
| Cytoplasm_Intensity_UpperQuartileIntensity_Mito | 4.9E−04 | 5.0E−05 | 4.5E+00 |
| Cytoplasm_Location_Center_Y | 9.0E−04 | 9.0E−05 | 4.2E+00 |
| Cytoplasm_Location_CenterMassIntensity_Y_AGP | 9.0E−04 | 9.0E−05 | 4.2E+00 |
| Cytoplasm_Location_CenterMassIntensity_Y_Lipid | 8.6E−04 | 8.5E−05 | 4.2E+00 |
| Cytoplasm_Location_CenterMassIntensity_Y_DNA | 9.2E−04 | 9.1E−05 | 4.2E+00 |
| Cytoplasm_Location_CenterMassIntensity_Y_Mito | 8.3E−04 | 8.3E−05 | 4.2E+00 |
| Cytoplasm_Location_MaxIntensity_Y_AGP | 1.5E−03 | 1.4E−04 | 3.9E+00 |
| Cytoplasm_Location_MaxIntensity_Y_Lipid | 1.0E−03 | 9.8E−05 | 4.1E+00 |
| Cytoplasm_Location_MaxIntensity_Y_DNA | 1.6E−03 | 1.5E−04 | 3.9E+00 |
| Cytoplasm_Location_MaxIntensity_Y_Mito | 1.2E−03 | 1.2E−04 | 4.0E+00 |
| Cytoplasm_Number_Object_Number | 6.0E−09 | 1.7E−09 | −1.2E+01 |
| Cytoplasm_Parent_Cells | 6.1E−09 | 1.8E−09 | −1.2E+01 |
| Cytoplasm_Parent_Nuclei | 6.1E−09 | 1.8E−09 | −1.2E+01 |
| Cytoplasm_RadialDistribution_FracAtD_AGP_1of4 | 2.3E−14 | 3.4E−14 | 3.1E+01 |
| Cytoplasm_RadialDistribution_FracAtD_Lipid_2of4 | 5.3E−14 | 6.9E−14 | 2.9E+01 |
| Cytoplasm_RadialDistribution_FracAtD_DNA_1of4 | 7.4E−08 | 1.7E−08 | 1.0E+01 |
| Cytoplasm_RadialDistribution_FracAtD_Mito_1of4 | 3.2E−14 | 4.6E−14 | 3.1E+01 |
| Cytoplasm_RadialDistribution_MeanFrac_AGP_1of4 | 6.2E−14 | 7.8E−14 | 2.9E+01 |
| Cytoplasm_RadialDistribution_MeanFrac_Lipid_4of4 | 2.2E−16 | 9.4E−16 | −4.4E+01 |
| Cytoplasm_RadialDistribution_MeanFrac_DNA_2of4 | 3.1E−09 | 1.0E−09 | 1.3E+01 |
| Cytoplasm_RadialDistribution_MeanFrac_Mito_1of4 | 1.5E−15 | 4.3E−15 | 3.8E+01 |
| Cytoplasm_RadialDistribution_RadialCV_AGP_4of4 | 8.0E−08 | 1.8E−08 | 1.0E+01 |
| Cytoplasm_RadialDistribution_RadialCV_Lipid_4of4 | 7.3E−09 | 2.1E−09 | 1.2E+01 |
| Cytoplasm_RadialDistribution_RadialCV_DNA_4of4 | 1.4E−14 | 2.3E−14 | 3.2E+01 |
| Cytoplasm_RadialDistribution_RadialCV_Mito_4of4 | 2.3E−16 | 9.4E−16 | 4.4E+01 |
| Cytoplasm_Texture_AngularSecondMoment_AGP_5_01 | 4.8E−08 | 1.2E−08 | −1.1E+01 |
| Cytoplasm_Texture_AngularSecondMoment_DNA_20_00 | 1.7E−05 | 2.3E−06 | −6.4E+00 |
| Cytoplasm_Texture_AngularSecondMoment_Mito_20_02 | 3.3E−04 | 3.5E−05 | −4.7E+00 |
| Cytoplasm_Texture_Contrast_AGP_5_00 | 1.0E−06 | 1.8E−07 | 8.2E+00 |
| Cytoplasm_Texture_Contrast_Lipid_5_02 | 2.4E−05 | 3.1E−06 | −6.2E+00 |
| Cytoplasm_Texture_Contrast_DNA_20_03 | 5.6E−03 | 4.9E−04 | 3.3E+00 |
| Cytoplasm_Texture_Contrast_Mito_10_03 | 1.6E−06 | 2.6E−07 | 7.9E+00 |
| Cytoplasm_Texture_Correlation_AGP_10_03 | 1.1E−10 | 5.2E−11 | −1.7E+01 |
| Cytoplasm_Texture_Correlation_Lipid_20_01 | 1.1E−13 | 1.2E−13 | −2.8E+01 |
| Cytoplasm_Texture_Correlation_DNA_20_03 | 6.8E−15 | 1.3E−14 | −3.4E+01 |
| Cytoplasm_Texture_Correlation_Mito_20_01 | 3.8E−15 | 8.5E−15 | −3.6E+01 |
| Cytoplasm_Texture_DifferenceEntropy_AGP_5_00 | 6.0E−08 | 1.4E−08 | 1.0E+01 |
| Cytoplasm_Texture_DifferenceEntropy_Lipid_20_01 | 2.8E−03 | 2.5E−04 | −3.6E+00 |
| Cytoplasm_Texture_DifferenceEntropy_DNA_10_01 | 7.7E−04 | 7.7E−05 | 4.3E+00 |
| Cytoplasm_Texture_DifferenceEntropy_Mito_10_01 | 3.6E−06 | 5.7E−07 | 7.3E+00 |
| Cytoplasm_Texture_DifferenceVariance_AGP_10_00 | 3.9E−06 | 6.1E−07 | −7.3E+00 |
| Cytoplasm_Texture_DifferenceVariance_Lipid_5_00 | 1.5E−03 | 1.4E−04 | 3.9E+00 |
| Cytoplasm_Texture_DifferenceVariance_DNA_5_00 | 2.2E−03 | 2.0E−04 | 3.7E+00 |
| Cytoplasm_Texture_DifferenceVariance_Mito_20_02 | 2.0E−04 | 2.1E−05 | −5.0E+00 |
| Cytoplasm_Texture_Entropy_AGP_5_00 | 6.3E−09 | 1.8E−09 | 1.2E+01 |
| Cytoplasm_Texture_Entropy_Lipid_20_01 | 1.5E−03 | 1.4E−04 | −3.9E+00 |
| Cytoplasm_Texture_Entropy_DNA_10_01 | 8.9E−05 | 1.0E−05 | 5.4E+00 |
| Cytoplasm_Texture_Entropy_Mito_10_02 | 1.3E−05 | 1.7E−06 | 6.6E+00 |
| Cytoplasm_Texture_InfoMeas1_AGP_20_01 | 2.8E−10 | 1.2E−10 | −1.6E+01 |
| Cytoplasm_Texture_InfoMeas1_Lipid_5_00 | 7.6E−04 | 7.6E−05 | −4.3E+00 |
| Cytoplasm_Texture_InfoMeas1_DNA_20_03 | 2.3E−13 | 2.4E−13 | −2.7E+01 |
| Cytoplasm_Texture_InfoMeas1_Mito_5_00 | 1.0E−14 | 1.9E−14 | −3.3E+01 |
| Cytoplasm_Texture_InverseDifferenceMoment_AGP_20_03 | 3.6E−10 | 1.5E−10 | −1.5E+01 |
| Cytoplasm_Texture_InverseDifferenceMoment_Lipid_20_00 | 3.6E−04 | 3.8E−05 | −4.7E+00 |
| Cytoplasm_Texture_InverseDifferenceMoment_DNA_20_03 | 9.0E−09 | 2.5E−09 | −1.2E+01 |
| Cytoplasm_Texture_InverseDifferenceMoment_Mito_20_01 | 6.6E−09 | 1.9E−09 | −1.2E+01 |
| Cytoplasm_Texture_SumAverage_AGP_5_00 | 2.4E−07 | 4.9E−08 | 9.2E+00 |
| Cytoplasm_Texture_SumAverage_Lipid_20_01 | 1.1E−06 | 1.9E−07 | −8.2E+00 |
| Cytoplasm_Texture_SumAverage_DNA_20_03 | 5.2E−06 | 7.9E−07 | −7.1E+00 |
| Cytoplasm_Texture_SumEntropy_AGP_5_02 | 2.4E−08 | 6.1E−09 | 1.1E+01 |
| Cytoplasm_Texture_SumEntropy_Lipid_20_01 | 3.7E−04 | 3.9E−05 | −4.7E+00 |
| Cytoplasm_Texture_SumEntropy_DNA_5_02 | 1.3E−04 | 1.5E−05 | 5.2E+00 |
| Cytoplasm_Texture_SumEntropy_Mito_5_00 | 1.8E−06 | 3.0E−07 | 7.8E+00 |
| Cytoplasm_Texture_SumVariance_AGP_5_00 | 1.7E−04 | 1.8E−05 | 5.1E+00 |
| Cytoplasm_Texture_SumVariance_Lipid_20_01 | 1.6E−05 | 2.1E−06 | −6.4E+00 |
| Cytoplasm_Texture_SumVariance_DNA_5_02 | 8.9E−03 | 7.6E−04 | 3.0E+00 |
| Cytoplasm_Texture_SumVariance_Mito_5_00 | 1.9E−06 | 3.1E−07 | 7.8E+00 |
| Cytoplasm_Texture_Variance_AGP_5_03 | 4.9E−05 | 6.0E−06 | 5.8E+00 |
| Cytoplasm_Texture_Variance_Lipid_20_01 | 3.3E−05 | 4.2E−06 | −6.0E+00 |
| Cytoplasm_Texture_Variance_Mito_10_03 | 2.9E−06 | 4.7E−07 | 7.5E+00 |
| Nuclei_AreaShape_Center_Y | 9.7E−04 | 9.6E−05 | 4.2E+00 |
| Nuclei_AreaShape_Compactness | 1.5E−12 | 1.1E−12 | 2.3E+01 |
| Nuclei_AreaShape_Eccentricity | 5.5E−17 | 3.8E−16 | 4.8E+01 |
| Nuclei_AreaShape_EulerNumber | 1.3E−06 | 2.2E−07 | −8.0E+00 |
| Nuclei_AreaShape_Extent | 4.0E−14 | 5.5E−14 | −3.0E+01 |
| Nuclei_AreaShape_FormFactor | 1.8E−13 | 1.9E−13 | −2.7E+01 |
| Nuclei_AreaShape_MajorAxisLength | 1.7E−03 | 1.6E−04 | 3.9E+00 |
| Nuclei_AreaShape_MaxFeretDiameter | 6.8E−04 | 6.9E−05 | 4.3E+00 |
| Nuclei_AreaShape_MaximumRadius | 2.4E−07 | 4.9E−08 | −9.3E+00 |
| Nuclei_AreaShape_MeanRadius | 1.8E−05 | 2.4E−06 | −6.3E+00 |
| Nuclei_AreaShape_MedianRadius | 1.3E−04 | 1.5E−05 | −5.2E+00 |
| Nuclei_AreaShape_MinFeretDiameter | 2.8E−05 | 3.6E−06 | −6.1E+00 |
| Nuclei_AreaShape_MinorAxisLength | 3.8E−06 | 6.0E−07 | −7.3E+00 |
| Nuclei_AreaShape_Solidity | 6.2E−13 | 5.5E−13 | −2.5E+01 |
| Nuclei_AreaShape_Zernike_4_4 | 4.4E−18 | 5.8E−17 | 5.8E+01 |
| Nuclei_Correlation_Correlation_Lipid_AGP | 1.8E−08 | 4.6E−09 | −1.1E+01 |
| Nuclei_Correlation_Correlation_DNA_AGP | 2.2E−12 | 1.5E−12 | −2.3E+01 |
| Nuclei_Correlation_Correlation_DNA_Lipid | 1.9E−07 | 3.9E−08 | −9.4E+00 |
| Nuclei_Correlation_Correlation_DNA_Mito | 3.7E−07 | 7.2E−08 | 8.9E+00 |
| Nuclei_Correlation_Correlation_Mito_AGP | 8.3E−10 | 3.1E−10 | 1.4E+01 |
| Nuclei_Correlation_Correlation_Mito_Lipid | 5.4E−07 | 1.0E−07 | 8.7E+00 |
| Nuclei_Correlation_K_AGP_Lipid | 2.5E−09 | 8.1E−10 | 1.3E+01 |
| Nuclei_Correlation_K_AGP_DNA | 6.0E−06 | 8.9E−07 | 7.0E+00 |
| Nuclei_Correlation_K_AGP_Mito | 1.5E−08 | 3.9E−09 | 1.2E+01 |
| Nuclei_Correlation_K_Lipid_AGP | 5.5E−11 | 2.8E−11 | −1.8E+01 |
| Nuclei_Correlation_K_Lipid_DNA | 3.0E−05 | 3.9E−06 | −6.0E+00 |
| Nuclei_Correlation_K_Lipid_Mito | 9.8E−08 | 2.2E−08 | 1.0E+01 |
| Nuclei_Correlation_K_DNA_AGP | 5.7E−08 | 1.3E−08 | −1.0E+01 |
| Nuclei_Correlation_K_DNA_Lipid | 2.5E−03 | 2.2E−04 | 3.7E+00 |
| Nuclei_Correlation_K_DNA_Mito | 1.0E−07 | 2.2E−08 | 9.9E+00 |
| Nuclei_Correlation_K_Mito_AGP | 3.5E−11 | 1.9E−11 | −1.8E+01 |
| Nuclei_Correlation_K_Mito_Lipid | 6.7E−08 | 1.6E−08 | −1.0E+01 |
| Nuclei_Correlation_K_Mito_DNA | 9.3E−09 | 2.6E−09 | −1.2E+01 |
| Nuclei_Correlation_Overlap_Lipid_AGP | 2.0E−06 | 3.3E−07 | −7.7E+00 |
| Nuclei_Correlation_Overlap_DNA_AGP | 2.8E−13 | 2.8E−13 | −2.6E+01 |
| Nuclei_Correlation_Overlap_DNA_Lipid | 2.7E−10 | 1.1E−10 | −1.6E+01 |
| Nuclei_Correlation_Overlap_DNA_Mito | 2.3E−08 | 5.8E−09 | −1.1E+01 |
| Nuclei_Correlation_Overlap_Mito_AGP | 4.8E−07 | 9.0E−08 | 8.7E+00 |
| Nuclei_Granularity_10_AGP | 9.4E−13 | 7.8E−13 | 2.4E+01 |
| Nuclei_Granularity_10_Lipid | 2.8E−09 | 9.0E−10 | 1.3E+01 |
| Nuclei_Granularity_10_DNA | 1.1E−13 | 1.2E−13 | 2.8E+01 |
| Nuclei_Granularity_10_Mito | 9.5E−14 | 1.1E−13 | 2.8E+01 |
| Nuclei_Granularity_2_AGP | 1.7E−16 | 7.9E−16 | −4.5E+01 |
| Nuclei_Granularity_4_Lipid | 4.2E−15 | 9.3E−15 | 3.5E+01 |
| Nuclei_Granularity_5_DNA | 6.8E−20 | 4.3E−18 | 7.8E+01 |
| Nuclei_Granularity_4_Mito | 8.1E−19 | 2.8E−17 | 6.6E+01 |
| Nuclei_Intensity_IntegratedIntensity_AGP | 1.1E−10 | 5.1E−11 | −1.7E+01 |
| Nuclei_Intensity_IntegratedIntensity_Lipid | 5.9E−05 | 7.1E−06 | −5.7E+00 |
| Nuclei_Intensity_IntegratedIntensity_DNA | 3.3E−09 | 1.0E−09 | −1.3E+01 |
| Nuclei_Intensity_IntegratedIntensity_Mito | 4.2E−06 | 6.4E−07 | 7.3E+00 |
| Nuclei_Intensity_IntegratedIntensityEdge_AGP | 1.0E−06 | 1.8E−07 | −8.2E+00 |
| Nuclei_Intensity_IntegratedIntensityEdge_DNA | 5.1E−11 | 2.6E−11 | −1.8E+01 |
| Nuclei_Intensity_IntegratedIntensityEdge_Mito | 1.2E−04 | 1.3E−05 | 5.3E+00 |
| Nuclei_Intensity_LowerQuartileIntensity_AGP | 1.4E−09 | 4.9E−10 | −1.4E+01 |
| Nuclei_Intensity_LowerQuartileIntensity_Lipid | 9.0E−05 | 1.0E−05 | −5.4E+00 |
| Nuclei_Intensity_LowerQuartileIntensity_DNA | 2.2E−11 | 1.2E−11 | −1.9E+01 |
| Nuclei_Intensity_LowerQuartileIntensity_Mito | 2.3E−06 | 3.7E−07 | 7.7E+00 |
| Nuclei_Intensity_MADIntensity_AGP | 3.2E−12 | 2.2E−12 | −2.2E+01 |
| Nuclei_Intensity_MADIntensity_DNA | 3.6E−04 | 3.8E−05 | 4.7E+00 |
| Nuclei_Intensity_MADIntensity_Mito | 9.2E−09 | 2.6E−09 | 1.2E+01 |
| Nuclei_Intensity_MassDisplacement_AGP | 1.9E−11 | 1.1E−11 | 1.9E+01 |
| Nuclei_Intensity_MassDisplacement_Lipid | 2.6E−13 | 2.6E−13 | 2.6E+01 |
| Nuclei_Intensity_MassDisplacement_DNA | 1.7E−18 | 3.2E−17 | 6.2E+01 |
| Nuclei_Intensity_MassDisplacement_Mito | 1.6E−08 | 4.2E−09 | 1.2E+01 |
| Nuclei_Intensity_MaxIntensity_AGP | 2.4E−10 | 1.0E−10 | −1.6E+01 |
| Nuclei_Intensity_MaxIntensity_Mito | 3.6E−03 | 3.2E−04 | 3.5E+00 |
| Nuclei_Intensity_MaxIntensityEdge_AGP | 1.3E−06 | 2.3E−07 | −8.0E+00 |
| Nuclei_Intensity_MaxIntensityEdge_DNA | 4.5E−09 | 1.4E−09 | −1.3E+01 |
| Nuclei_Intensity_MaxIntensityEdge_Mito | 5.6E−03 | 4.9E−04 | 3.3E+00 |
| Nuclei_Intensity_MeanIntensity_AGP | 1.7E−10 | 7.7E−11 | −1.6E+01 |
| Nuclei_Intensity_MeanIntensity_Lipid | 4.3E−04 | 4.5E−05 | −4.6E+00 |
| Nuclei_Intensity_MeanIntensity_DNA | 1.9E−07 | 3.9E−08 | −9.5E+00 |
| Nuclei_Intensity_MeanIntensity_Mito | 1.7E−06 | 2.9E−07 | 7.8E+00 |
| Nuclei_Intensity_MeanIntensityEdge_AGP | 9.9E−07 | 1.8E−07 | −8.2E+00 |
| Nuclei_Intensity_MeanIntensityEdge_DNA | 2.3E−10 | 9.7E−11 | −1.6E+01 |
| Nuclei_Intensity_MeanIntensityEdge_Mito | 1.4E−04 | 1.5E−05 | 5.2E+00 |
| Nuclei_Intensity_MedianIntensity_AGP | 1.0E−10 | 4.8E−11 | −1.7E+01 |
| Nuclei_Intensity_MedianIntensity_Lipid | 5.3E−04 | 5.4E−05 | −4.5E+00 |
| Nuclei_Intensity_MedianIntensity_DNA | 2.5E−09 | 8.2E−10 | −1.3E+01 |
| Nuclei_Intensity_MedianIntensity_Mito | 3.7E−07 | 7.3E−08 | 8.9E+00 |
| Nuclei_Intensity_MinIntensity_AGP | 1.8E−06 | 3.0E−07 | −7.8E+00 |
| Nuclei_Intensity_MinIntensity_Lipid | 5.6E−07 | 1.0E−07 | −8.6E+00 |
| Nuclei_Intensity_MinIntensity_DNA | 2.8E−10 | 1.2E−10 | −1.6E+01 |
| Nuclei_Intensity_MinIntensity_Mito | 5.3E−04 | 5.4E−05 | 4.5E+00 |
| Nuclei_Intensity_MinIntensityEdge_AGP | 3.1E−06 | 5.0E−07 | −7.5E+00 |
| Nuclei_Intensity_MinIntensityEdge_Lipid | 6.9E−07 | 1.3E−07 | −8.5E+00 |
| Nuclei_Intensity_MinIntensityEdge_DNA | 2.9E−10 | 1.2E−10 | −1.6E+01 |
| Nuclei_Intensity_MinIntensityEdge_Mito | 8.8E−03 | 7.6E−04 | 3.0E+00 |
| Nuclei_Intensity_StdIntensity_AGP | 4.6E−11 | 2.4E−11 | −1.8E+01 |
| Nuclei_Intensity_StdIntensity_DNA | 3.5E−03 | 3.2E−04 | 3.5E+00 |
| Nuclei_Intensity_StdIntensity_Mito | 2.6E−05 | 3.3E−06 | 6.1E+00 |
| Nuclei_Intensity_StdIntensityEdge_AGP | 3.1E−06 | 5.0E−07 | −7.5E+00 |
| Nuclei_Intensity_StdIntensityEdge_Lipid | 1.9E−05 | 2.4E−06 | 6.3E+00 |
| Nuclei_Intensity_StdIntensityEdge_DNA | 7.2E−08 | 1.7E−08 | −1.0E+01 |
| Nuclei_Intensity_StdIntensityEdge_Mito | 1.3E−04 | 1.5E−05 | 5.2E+00 |
| Nuclei_Intensity_UpperQuartileIntensity_AGP | 5.1E−11 | 2.6E−11 | −1.8E+01 |
| Nuclei_Intensity_UpperQuartileIntensity_Lipid | 6.4E−04 | 6.5E−05 | −4.4E+00 |
| Nuclei_Intensity_UpperQuartileIntensity_DNA | 5.8E−05 | 7.0E−06 | −5.7E+00 |
| Nuclei_Intensity_UpperQuartileIntensity_Mito | 7.1E−07 | 1.3E−07 | 8.5E+00 |
| Nuclei_Location_Center_Y | 9.8E−04 | 9.6E−05 | 4.2E+00 |
| Nuclei_Location_CenterMassIntensity_Y_AGP | 1.0E−03 | 9.8E−05 | 4.1E+00 |
| Nuclei_Location_CenterMassIntensity_Y_Lipid | 9.9E−04 | 9.7E−05 | 4.1E+00 |
| Nuclei_Location_CenterMassIntensity_Y_DNA | 9.8E−04 | 9.6E−05 | 4.2E+00 |
| Nuclei_Location_CenterMassIntensity_Y_Mito | 9.3E−04 | 9.1E−05 | 4.2E+00 |
| Nuclei_Location_MaxIntensity_Y_AGP | 1.1E−03 | 1.0E−04 | 4.1E+00 |
| Nuclei_Location_MaxIntensity_Y_Lipid | 1.1E−03 | 1.0E−04 | 4.1E+00 |
| Nuclei_Location_MaxIntensity_Y_DNA | 9.3E−04 | 9.2E−05 | 4.2E+00 |
| Nuclei_Location_MaxIntensity_Y_Mito | 5.1E−04 | 5.3E−05 | 4.5E+00 |
| Nuclei_Neighbors_AngleBetweenNeighbors_2 | 2.3E−12 | 1.6E−12 | −2.2E+01 |
| Nuclei_Neighbors_FirstClosestDistance_2 | 3.6E−03 | 3.2E−04 | −3.5E+00 |
| Nuclei_Neighbors_FirstClosestObjectNumber_2 | 5.7E−09 | 1.7E−09 | −1.2E+01 |
| Nuclei_Neighbors_NumberOfNeighbors_2 | 2.0E−11 | 1.1E−11 | 1.9E+01 |
| Nuclei_Neighbors_PercentTouching_2 | 1.0E−11 | 6.4E−12 | 2.0E+01 |
| Nuclei_Neighbors_SecondClosestDistance_2 | 4.8E−04 | 4.9E−05 | 4.5E+00 |
| Nuclei_Neighbors_SecondClosestObjectNumber_2 | 5.6E−09 | 1.7E−09 | −1.2E+01 |
| Nuclei_Number_Object_Number | 6.0E−09 | 1.7E−09 | −1.2E+01 |
| Nuclei_RadialDistribution_FracAtD_AGP_3of4 | 2.2E−13 | 2.3E−13 | −2.7E+01 |
| Nuclei_RadialDistribution_FracAtD_Lipid_1of4 | 6.7E−09 | 1.9E−09 | −1.2E+01 |
| Nuclei_RadialDistribution_FracAtD_DNA_2of4 | 4.4E−14 | 6.0E−14 | 3.0E+01 |
| Nuclei_RadialDistribution_FracAtD_Mito_3of4 | 6.9E−13 | 6.0E−13 | 2.4E+01 |
| Nuclei_RadialDistribution_MeanFrac_AGP_3of4 | 1.7E−13 | 1.8E−13 | −2.7E+01 |
| Nuclei_RadialDistribution_MeanFrac_Lipid_2of4 | 1.6E−06 | 2.7E−07 | −7.9E+00 |
| Nuclei_RadialDistribution_MeanFrac_DNA_2of4 | 1.6E−15 | 4.4E−15 | 3.8E+01 |
| Nuclei_Radial Distribution_MeanFrac_Mito_3of4 | 3.7E−13 | 3.5E−13 | 2.6E+01 |
| Nuclei_RadialDistribution_RadialCV_AGP_3of4 | 4.6E−13 | 4.2E−13 | 2.5E+01 |
| Nuclei_RadialDistribution_RadialCV_Lipid_3of4 | 2.7E−12 | 1.9E−12 | 2.2E+01 |
| Nuclei_RadialDistribution_RadialCV_DNA_3of4 | 3.4E−21 | 6.3E−19 | 9.7E+01 |
| Nuclei_RadialDistribution_RadialCV_Mito_2of4 | 2.5E−15 | 5.9E−15 | 3.7E+01 |
| Nuclei_Texture_AngularSecondMoment_AGP_10_02 | 6.0E−09 | 1.8E−09 | 1.2E+01 |
| Nuclei_Texture_AngularSecondMoment_Lipid_5_03 | 8.4E−04 | 8.4E−05 | 4.2E+00 |
| Nuclei_Texture_AngularSecondMoment_DNA_20_03 | 1.9E−07 | 4.0E−08 | 9.4E+00 |
| Nuclei_Texture_AngularSecondMoment_Mito_5_02 | 4.4E−11 | 2.3E−11 | −1.8E+01 |
| Nuclei_Texture_Contrast_AGP_10_03 | 5.9E−11 | 3.0E−11 | −1.8E+01 |
| Nuclei_Texture_Contrast_Lipid_20_02 | 6.8E−04 | 6.9E−05 | 4.3E+00 |
| Nuclei_Texture_Contrast_DNA_5_01 | 4.6E−05 | 5.7E−06 | 5.8E+00 |
| Nuclei_Texture_Contrast_Mito_5_03 | 9.2E−06 | 1.3E−06 | 6.8E+00 |
| Nuclei_Texture_Correlation_AGP_10_03 | 3.7E−14 | 5.2E−14 | 3.0E+01 |
| Nuclei_Texture_Correlation_Lipid_5_03 | 1.4E−12 | 1.0E−12 | 2.3E+01 |
| Nuclei_Texture_Correlation_DNA_10_00 | 2.1E−10 | 9.1E−11 | −1.6E+01 |
| Nuclei_Texture_Correlation_Mito_5_02 | 7.5E−10 | 2.8E−10 | 1.5E+01 |
| Nuclei_Texture_DifferenceEntropy_AGP_10_00 | 6.5E−12 | 4.2E−12 | −2.1E+01 |
| Nuclei_Texture_DifferenceEntropy_Lipid_20_01 | 1.1E−06 | 1.9E−07 | 8.2E+00 |
| Nuclei_Texture_DifferenceEntropy_DNA_10_03 | 1.4E−06 | 2.4E−07 | −8.0E+00 |
| Nuclei_Texture_DifferenceEntropy_Mito_20_00 | 1.4E−06 | 2.4E−07 | 8.0E+00 |
| Nuclei_Texture_DifferenceVariance_AGP_10_00 | 1.2E−09 | 4.4E−10 | 1.4E+01 |
| Nuclei_Texture_DifferenceVariance_Lipid_20_00 | 6.5E−05 | 7.7E−06 | 5.6E+00 |
| Nuclei_Texture_DifferenceVariance_DNA_20_03 | 4.2E−06 | 6.4E−07 | 7.3E+00 |
| Nuclei_Texture_DifferenceVariance_Mito_10_00 | 1.2E−06 | 2.1E−07 | −8.1E+00 |
| Nuclei_Texture_Entropy_AGP_5_00 | 1.3E−10 | 5.9E−11 | −1.7E+01 |
| Nuclei_Texture_Entropy_Lipid_20_01 | 3.7E−06 | 5.8E−07 | 7.3E+00 |
| Nuclei_Texture_Entropy_DNA_10_03 | 2.0E−06 | 3.3E−07 | −7.7E+00 |
| Nuclei_Texture_Entropy_Mito_5_02 | 3.7E−09 | 1.2E−09 | 1.3E+01 |
| Nuclei_Texture_InfoMeas1_AGP_10_03 | 2.9E−13 | 2.8E−13 | 2.6E+01 |
| Nuclei_Texture_InfoMeas1_Lipid_20_03 | 6.0E−07 | 1.1E−07 | −8.6E+00 |
| Nuclei_Texture_InfoMeas2_DNA_10_03 | 3.1E−09 | 1.0E−09 | −1.3E+01 |
| Nuclei_Texture_InfoMeas2_Mito_5_01 | 3.4E−09 | 1.1E−09 | 1.3E+01 |
| Nuclei_Texture_InverseDifferenceMoment_AGP_10_00 | 7.1E−12 | 4.5E−12 | 2.1E+01 |
| Nuclei_Texture_InverseDifferenceMoment_Lipid_20_00 | 3.1E−06 | 5.0E−07 | 7.5E+00 |
| Nuclei_Texture_InverseDifferenceMoment_DNA_20_03 | 4.9E−10 | 1.9E−10 | 1.5E+01 |
| Nuclei_Texture_InverseDifferenceMoment_Mito_10_00 | 8.9E−10 | 3.3E−10 | −1.4E+01 |
| Nuclei_Texture_SumAverage_AGP_10_00 | 4.3E−11 | 2.2E−11 | −1.8E+01 |
| Nuclei_Texture_SumAverage_Lipid_10_00 | 1.2E−05 | 1.6E−06 | −6.6E+00 |
| Nuclei_Texture_SumAverage_DNA_10_01 | 3.5E−11 | 1.9E−11 | −1.8E+01 |
| Nuclei_Texture_SumAverage_Mito_20_00 | 6.9E−07 | 1.3E−07 | 8.5E+00 |
| Nuclei_Texture_SumEntropy_AGP_5_00 | 2.3E−11 | 1.3E−11 | −1.9E+01 |
| Nuclei_Texture_SumEntropy_Lipid_20_01 | 1.4E−06 | 2.4E−07 | 8.0E+00 |
| Nuclei_Texture_SumEntropy_DNA_10_03 | 1.8E−06 | 3.0E−07 | −7.8E+00 |
| Nuclei_Texture_SumEntropy_Mito_5_02 | 2.3E−08 | 5.9E−09 | 1.1E+01 |
| Nuclei_Texture_SumVariance_AGP_10_03 | 7.6E−11 | 3.7E−11 | −1.7E+01 |
| Nuclei_Texture_SumVariance_Lipid_20_01 | 3.1E−05 | 3.9E−06 | 6.0E+00 |
| Nuclei_Texture_SumVariance_DNA_5_02 | 2.3E−05 | 3.0E−06 | 6.2E+00 |
| Nuclei_Texture_SumVariance_Mito_5_02 | 3.7E−06 | 5.7E−07 | 7.3E+00 |
| Nuclei_Texture_Variance_AGP_10_03 | 6.0E−11 | 3.0E−11 | −1.8E+01 |
| Nuclei_Texture_Variance_Lipid_20_00 | 3.7E−04 | 3.9E−05 | 4.7E+00 |
| Nuclei_Texture_Variance_DNA_5_00 | 4.9E−05 | 6.0E−06 | 5.8E+00 |
| Nuclei_Texture_Variance_Mito_5_02 | 7.1E−06 | 1.0E−06 | 6.9E+00 |

TABLE 4
AMSCs summary statistics. Sc, subcutaneous adipose depot;
vc, visceral adipose depot; total, total number of samples.

| | | | |
|---|---|---|---|
| adipose depot | sc (N = 29) | vc (N = 36) | Total (N = 65) |

sex

| | | | |
|---|---|---|---|
| male | 6 (20.7%) | 9 (25.0%) | 15 (23.1%) |
| female | 23 (79.3%) | 27 (75.0%) | 50 (76.9%) |

T2D

| | | | |
|---|---|---|---|
| not reported | 1 | 2 | 3 |
| negative | 18 (64.3%) | 22 (64.7%) | 40 (64.5%) |
| positive | 10 (35.7%) | 12 (35.3%) | 22 (35.5%) |

WHR

| | | | |
|---|---|---|---|
| not reported | 25 | 30 | 55 |
| Mean | 0.87 | 0.86 | 0.86 |
| Range | 0.77-0.92 | 0.77-0.92 | 0.77-0.92 |

age

| | | | |
|---|---|---|---|
| Mean | 41.83 | 43.28 | 42.63 |
| Range | 17−72 | 17−72 | 17−72 |

BMI

| | | | |
|---|---|---|---|
| not reported | 1 | 1 | 2 |
| Mean | 50.07 | 48.75 | 49.34 |
| Range | 22.27-69.26 | 22.27-83.26 | 22.27-83.26 |

(ANOVA adj. BMI, sex, age, batch; t-test, significance level FDR 5%

TABLE 5
Significant polygenic risk (PRS) effects on LipocyteProfiler for HOMA-IR and
WHRadjBMI in terminally differentiated AMSCs. (ANOVA adj. BMI, sex, age, batch,
significance level 5% FDR). P-value, p-value of ANOVA, q-value, q-value of ANOVA, FDR;
eta_sq, eta square of ANOVA, effect size; F value of ANOVA; t-statistics of t-test.

| | | | | | |
|---|---|---|---|---|---|
| LipocyteProfiler features | p-value | q-value | eta_sq | F value | t-statistics |

visceral AMSCs PRS HOMA−IR

| | | | | | |
|---|---|---|---|---|---|
| Cells_AreaShape_Zernike_8_4 | 0.00 | 0.03 | 0.24 | 14.37 | 2.28 |
| Cells_Correlation_K_Lipid_DNA | 0.01 | 0.04 | 0.35 | 10.99 | −2.91 |
| Cells_Correlation_Overlap_DNA_Lipid | 0.00 | 0.03 | 0.48 | 16.42 | −4.36 |
| Cells_Granularity_2_Lipid | 0.00 | 0.03 | 0.36 | 13.67 | 3.44 |
| Cells_Granularity_3_Lipid | 0.01 | 0.03 | 0.23 | 11.19 | 2.63 |
| Cells_Mean_LargeLipidObjects_Granularity_1_Lipid | 0.00 | 0.03 | 0.36 | 12.59 | 3.66 |
| Cells_Mean_LargeLipidObjects_Granularity_2_Lipid | 0.00 | 0.03 | 0.35 | 12.73 | 3.58 |
| Cells_Mean_LargeLipidObjects_Granularity_3_Lipid | 0.01 | 0.05 | 0.27 | 9.99 | 3.02 |
| Cells_RadialDistribution_FracAtD_DNA_3of4 | 0.01 | 0.04 | 0.38 | 10.50 | −3.69 |
| Cells_RadialDistribution_RadialCV_Lipid_2of4 | 0.01 | 0.04 | 0.28 | 10.75 | 2.87 |
| Cells_Texture_AngularSecondMoment_Lipid_5_03 | 0.00 | 0.00 | 0.44 | 41.18 | −3.77 |
| Cells_Texture_DifferenceEntropy_Lipid_10_01 | 0.00 | 0.03 | 0.33 | 13.95 | 3.12 |
| Cells_Texture_DifferenceVariance_Lipid_10_01 | 0.00 | 0.03 | 0.40 | 17.28 | −3.19 |
| Cells_Texture_Entropy_Lipid_10_00 | 0.00 | 0.03 | 0.34 | 13.31 | 3.20 |
| Cells_Texture_InfoMeas1_Lipid_5_00 | 0.01 | 0.03 | 0.33 | 11.23 | 3.15 |
| Cells_Texture_InverseDifferenceMoment_Lipid_10_01 | 0.00 | 0.03 | 0.37 | 18.38 | −3.50 |
| Cells_Texture_SumEntropy_Lipid_10_00 | 0.00 | 0.03 | 0.33 | 13.57 | 3.08 |
| Nuclei_Correlation_Correlation_DNA_Lipid | 0.00 | 0.03 | 0.41 | 12.12 | −3.45 |
| Nuclei_Correlation_Correlation_Mito_AGP | 0.00 | 0.03 | 0.45 | 17.16 | −3.67 |
| Nuclei_Correlation_Overlap_DNA_Lipid | 0.00 | 0.03 | 0.35 | 11.51 | −3.35 |
| Nuclei_Intensity_LowerQuartileIntensity_Lipid | 0.00 | 0.03 | 0.31 | 15.54 | 3.07 |
| Nuclei_Intensity_MinIntensityEdge_Lipid | 0.00 | 0.03 | 0.37 | 12.90 | 3.50 |
| Nuclei_Intensity_MinIntensity_Lipid | 0.00 | 0.03 | 0.37 | 13.16 | 3.50 |
| Nuclei_RadialDistribution_MeanFrac_Lipid_1of4 | 0.00 | 0.03 | 0.45 | 13.80 | −3.70 |
| Nuclei_RadialDistribution_MeanFrac_Lipid_2of4 | 0.00 | 0.03 | 0.39 | 11.50 | −3.47 |
| Nuclei_RadialDistribution_RadialCV_Lipid_4of4 | 0.00 | 0.03 | 0.33 | 11.73 | 3.20 |
| Nuclei_Texture_AngularSecondMoment_Lipid_10_01 | 0.00 | 0.03 | 0.28 | 12.96 | −2.72 |
| Nuclei_Texture_DifferenceVariance_Lipid_5_00 | 0.00 | 0.03 | 0.34 | 14.55 | −2.99 |
| Nuclei_Texture_InfoMeas1_Lipid_10_03 | 0.01 | 0.03 | 0.19 | 11.18 | −2.29 |
| Nuclei_Texture_InfoMeas2_Lipid_10_03 | 0.00 | 0.03 | 0.23 | 14.71 | 2.45 |
| Nuclei_Texture_SumEntropy_Lipid_10_01 | 0.01 | 0.04 | 0.29 | 10.07 | 2.87 |
| Cytoplasm_Granularity_2_Lipid | 0.00 | 0.02 | 0.45 | 21.51 | 4.15 |
| Cytoplasm_Granularity_3_Lipid | 0.00 | 0.02 | 0.38 | 21.90 | 3.76 |
| Cytoplasm_RadialDistribution_RadialCV_Lipid_2of4 | 0.01 | 0.04 | 0.30 | 10.71 | 2.83 |
| Cytoplasm_RadialDistribution_RadialCV_Lipid_3of4 | 0.01 | 0.03 | 0.32 | 11.16 | 2.99 |
| Cytoplasm_Texture_AngularSecondMoment_Lipid_5_01 | 0.00 | 0.00 | 0.45 | 36.97 | −3.77 |
| Cytoplasm_Texture_DifferenceEntropy_Lipid_10_02 | 0.00 | 0.03 | 0.35 | 13.97 | 3.24 |
| Cytoplasm_Texture_DifferenceVariance_Lipid_10_01 | 0.00 | 0.03 | 0.37 | 12.69 | −2.98 |
| Cytoplasm_Texture_Entropy_Lipid_10_00 | 0.00 | 0.03 | 0.34 | 13.01 | 3.22 |
| Cytoplasm_Texture_InverseDifferenceMoment_Lipid_10_01 | 0.00 | 0.03 | 0.38 | 17.31 | −3.52 |
| Cytoplasm_Texture_SumEntropy_Lipid_10_02 | 0.00 | 0.03 | 0.33 | 13.60 | 3.14 |

subcutaneous female AMSCs WHRadjBMI

| | | | | | |
|---|---|---|---|---|---|
| Cells_AreaShape_Compactness | 0.01 | 0.04 | 0.43 | 13.48 | 2.45 |
| Cells_AreaShape_Eccentricity | 0.02 | 0.04 | 0.57 | 10.90 | 3.24 |
| Cells_AreaShape_Extent | 0.00 | 0.04 | 0.38 | 31.42 | −2.19 |
| Cells_AreaShape_FormFactor | 0.01 | 0.04 | 0.47 | 20.28 | −2.64 |
| Cells_AreaShape_Solidity | 0.02 | 0.04 | 0.25 | 11.66 | −1.65 |
| Cells_AreaShape_Zernike_4_0 | 0.00 | 0.04 | 0.67 | 49.92 | 4.01 |
| Cells_Children_LargeLipidObjects_Count | 0.01 | 0.04 | 0.23 | 14.14 | −1.54 |
| Cells_Correlation_Correlation_DNA_Lipid | 0.00 | 0.04 | 0.27 | 25.16 | 1.74 |
| Cells_Correlation_Correlation_Mito_Lipid | 0.01 | 0.04 | 0.30 | 15.77 | 1.84 |
| Cells_Correlation_K_Lipid_AGP | 0.00 | 0.04 | 0.32 | 26.60 | 1.95 |
| Cells_Correlation_Overlap_Lipid_AGP | 0.01 | 0.04 | 0.27 | 17.84 | 1.71 |
| Cells_Correlation_Overlap_DNA_Lipid | 0.04 | 0.05 | 0.16 | 7.99 | 1.22 |
| Cells_Correlation_Overlap_Mito_Lipid | 0.02 | 0.04 | 0.36 | 12.37 | 2.10 |
| Cells_Granularity_1_Mito | 0.03 | 0.04 | 0.25 | 8.78 | 1.62 |
| Cells_Intensity_LowerQuartileIntensity_Lipid | 0.01 | 0.04 | 0.28 | 20.73 | −1.78 |
| Cells_Intensity_LowerQuartileIntensity_Mito | 0.02 | 0.04 | 0.28 | 11.31 | −1.77 |
| Cells_Intensity_MADIntensity_Lipid | 0.02 | 0.04 | 0.26 | 11.09 | −1.66 |
| Cells_Intensity_MADIntensity_Mito | 0.04 | 0.05 | 0.34 | 8.01 | −2.05 |
| Cells_Intensity_MassDisplacement_Lipid | 0.03 | 0.04 | 0.25 | 9.14 | 1.61 |
| Cells_Intensity_MassDisplacement_DNA | 0.01 | 0.04 | 0.29 | 20.52 | 1.79 |
| Cells_Intensity_MeanIntensity_Lipid | 0.02 | 0.04 | 0.28 | 11.23 | −1.75 |
| Cells_Intensity_MeanIntensity_Mito | 0.03 | 0.05 | 0.30 | 8.50 | −1.85 |
| Cells_Intensity_MeanIntensityEdge_Lipid | 0.00 | 0.04 | 0.23 | 27.67 | −1.55 |
| Cells_Intensity_MeanIntensityEdge_DNA | 0.03 | 0.05 | 0.23 | 8.27 | −1.53 |
| Cells_Intensity_MeanIntensityEdge_Mito | 0.03 | 0.05 | 0.29 | 8.32 | −1.81 |
| Cells_Intensity_MedianIntensity_Lipid | 0.01 | 0.04 | 0.26 | 13.65 | −1.68 |
| Cells_Intensity_MedianIntensity_Mito | 0.02 | 0.04 | 0.34 | 12.26 | −2.04 |
| Cells_Intensity_MinIntensity_Lipid | 0.00 | 0.04 | 0.38 | 59.41 | −2.22 |
| Cells_Intensity_MinIntensity_DNA | 0.04 | 0.05 | 0.19 | 7.86 | −1.37 |
| Cells_Intensity_MinIntensity_Mito | 0.01 | 0.04 | 0.34 | 17.84 | −2.02 |
| Cells_Intensity_MinIntensityEdge_Lipid | 0.00 | 0.04 | 0.38 | 60.88 | −2.19 |
| Cells_Intensity_MinIntensityEdge_Mito | 0.01 | 0.04 | 0.34 | 19.07 | −2.02 |
| Cells_Intensity_StdIntensityEdge_Lipid | 0.03 | 0.04 | 0.14 | 8.86 | −1.14 |
| Cells_Intensity_StdIntensityEdge_DNA | 0.03 | 0.04 | 0.34 | 9.03 | −2.03 |
| Cells_Intensity_UpperQuartileIntensity_Lipid | 0.03 | 0.04 | 0.28 | 9.83 | −1.78 |
| Cells_Intensity_UpperQuartileIntensity_Mito | 0.02 | 0.04 | 0.36 | 10.48 | −2.14 |
| Cells_Mean_LargeLipidObjects_Correlation_K_AGP_Lipid | 0.01 | 0.04 | 0.56 | 18.77 | −3.18 |
| Cells_Mean_LargeLipidObjects_Correlation_K_Lipid_AGP | 0.02 | 0.04 | 0.43 | 11.70 | 2.47 |
| Cells_Mean_LargeLipidObjects_Correlation_K_DNA_Mito | 0.02 | 0.04 | 0.40 | 11.13 | −2.30 |
| Cells_Mean_LargeLipidObjects_Correlation_K_Mito_AGP | 0.00 | 0.04 | 0.41 | 24.14 | 2.35 |
| Cells_Mean_LargeLipidObjects_Correlation_K_Mito_DNA | 0.03 | 0.04 | 0.40 | 8.93 | 2.32 |
| Cells_RadialDistribution_MeanFrac_AGP_1of4 | 0.02 | 0.04 | 0.33 | 10.63 | 1.97 |
| Cells_RadialDistribution_MeanFrac_DNA_1of4 | 0.01 | 0.04 | 0.34 | 15.13 | 2.03 |
| Cells_Texture_AngularSecondMoment_Lipid_20_01 | 0.00 | 0.04 | 0.29 | 23.53 | 1.80 |
| Cells Texture_AngularSecondMoment_Mito_5_02 | 0.00 | 0.04 | 0.39 | 33.77 | 2.25 |
| Cells_Texture_Correlation_Mito_20_02 | 0.02 | 0.04 | 0.56 | 13.12 | 3.16 |
| Cells_Texture_DifferenceEntropy_Lipid_20_01 | 0.03 | 0.04 | 0.24 | 9.44 | −1.58 |
| Cells_Texture_DifferenceEntropy_Mito_10_03 | 0.03 | 0.04 | 0.33 | 9.74 | −1.97 |
| Cells_Texture_DifferenceVariance_Lipid_10_01 | 0.01 | 0.04 | 0.30 | 13.87 | 1.84 |
| Cells_Texture_DifferenceVariance_Mito_10_03 | 0.01 | 0.04 | 0.28 | 14.35 | 1.77 |
| Cells_Texture_Entropy_Lipid_10_01 | 0.02 | 0.04 | 0.25 | 12.22 | −1.65 |
| Cells_Texture_Entropy_Mito_20_02 | 0.02 | 0.04 | 0.32 | 11.28 | −1.94 |
| Cells_Texture_InfoMeas2_Lipid_10_02 | 0.01 | 0.04 | 0.37 | 17.44 | −2.16 |
| Cells_Texture_InfoMeas1_DNA_5_01 | 0.01 | 0.04 | 0.54 | 14.37 | −3.07 |
| Cells_Texture_InverseDifferenceMoment_Lipid_10_01 | 0.02 | 0.04 | 0.25 | 11.91 | 1.65 |
| Cells_Texture_InverseDifferenceMoment_Mito_10_03 | 0.02 | 0.04 | 0.36 | 13.07 | 2.14 |
| Cells_Texture_SumAverage_Lipid_20_00 | 0.02 | 0.04 | 0.29 | 10.16 | −1.80 |
| Cells_Texture_SumAverage_Mito_20_00 | 0.03 | 0.04 | 0.31 | 8.90 | −1.88 |
| Cells_Texture_SumEntropy_Lipid_5_00 | 0.01 | 0.04 | 0.27 | 16.40 | −1.72 |
| Cells_Texture_SumEntropy_Mito_5_02 | 0.02 | 0.04 | 0.31 | 10.33 | −1.88 |
| Cytoplasm_AreaShape_Eccentricity | 0.02 | 0.04 | 0.60 | 11.97 | 3.44 |
| Cytoplasm_AreaShape_FormFactor | 0.02 | 0.04 | 0.24 | 11.40 | −1.59 |
| Cytoplasm_AreaShape_Zernike_7_7 | 0.00 | 0.04 | 0.56 | 24.79 | −3.16 |
| Cytoplasm_Correlation_Correlation_DNA_Lipid | 0.00 | 0.04 | 0.38 | 52.71 | 2.23 |
| Cytoplasm_Correlation_Correlation_Mito_Lipid | 0.01 | 0.04 | 0.33 | 19.17 | 1.99 |
| Cytoplasm_Correlation_K_Lipid_AGP | 0.02 | 0.04 | 0.31 | 12.79 | 1.89 |
| Cytoplasm_Correlation_Overlap_Lipid_AGP | 0.01 | 0.04 | 0.32 | 18.20 | 1.94 |
| Cytoplasm_Correlation_Overlap_Mito_Lipid | 0.00 | 0.04 | 0.40 | 26.24 | 2.32 |
| Cytoplasm_Granularity_3_AGP | 0.01 | 0.04 | 0.35 | 13.41 | −2.06 |
| Cytoplasm_Granularity_3_Mito | 0.02 | 0.04 | 0.35 | 12.36 | −2.06 |
| Cytoplasm_Intensity_IntegratedintensityEdge_Lipid | 0.02 | 0.04 | 0.17 | 11.58 | −1.29 |
| Cytoplasm_Intensity_LowerQuartileIntensity_Lipid | 0.01 | 0.04 | 0.30 | 15.75 | −1.83 |
| Cytoplasm_Intensity_LowerQuartileIntensity_Mito | 0.02 | 0.04 | 0.27 | 10.47 | −1.72 |
| Cytoplasm_Intensity_MADIntensity_Lipid | 0.02 | 0.04 | 0.26 | 12.62 | −1.68 |
| Cytoplasm_Intensity_MADIntensity_Mito | 0.03 | 0.04 | 0.35 | 8.59 | −2.05 |
| Cytoplasm_Intensity_MassDisplacement_DNA | 0.00 | 0.04 | 0.31 | 28.70 | 1.90 |
| Cytoplasm_Intensity_MaxIntensity_Lipid | 0.04 | 0.05 | 0.25 | 7.72 | −1.63 |
| Cytoplasm_Intensity_MeanIntensity_Lipid | 0.02 | 0.04 | 0.27 | 10.56 | −1.70 |
| Cytoplasm_Intensity_MeanIntensity_Mito | 0.04 | 0.05 | 0.28 | 8.22 | −1.77 |
| Cytoplasm_Intensity_MeanIntensityEdge_Lipid | 0.00 | 0.04 | 0.24 | 24.69 | −1.59 |
| Cytoplasm_Intensity_MeanIntensityEdge_DNA | 0.02 | 0.04 | 0.32 | 11.11 | −1.95 |
| Cytoplasm_Intensity_MeanIntensityEdge_Mito | 0.03 | 0.04 | 0.32 | 9.14 | −1.94 |
| Cytoplasm_Intensity_MedianIntensity_Lipid | 0.01 | 0.04 | 0.26 | 14.98 | −1.69 |
| Cytoplasm_Intensity_MedianIntensity_Mito | 0.02 | 0.04 | 0.32 | 11.84 | −1.93 |
| Cytoplasm_Intensity_MinIntensity_Lipid | 0.00 | 0.04 | 0.38 | 59.42 | −2.22 |
| Cytoplasm_Intensity_MinIntensity_DNA | 0.04 | 0.05 | 0.19 | 7.86 | −1.37 |
| Cytoplasm_Intensity_MinIntensity_Mito | 0.01 | 0.04 | 0.34 | 17.83 | −2.02 |
| Cytoplasm_Intensity_MinIntensityEdge_Lipid | 0.00 | 0.04 | 0.38 | 60.87 | −2.19 |
| Cytoplasm_Intensity_MinIntensityEdge_Mito | 0.01 | 0.04 | 0.34 | 19.10 | −2.02 |
| Cytoplasm_Intensity_UpperQuartileIntensity_Lipid | 0.03 | 0.04 | 0.28 | 9.13 | −1.75 |
| Cytoplasm_Intensity_UpperQuartileIntensity_Mito | 0.02 | 0.04 | 0.34 | 10.65 | −2.05 |
| Cytoplasm_RadialDistribution_FracAtD_Lipid_3of4 | 0.04 | 0.05 | 0.37 | 8.05 | −2.16 |
| Cytoplasm_RadialDistribution_MeanFrac_AGP_1of4 | 0.01 | 0.04 | 0.47 | 14.73 | 2.66 |
| Cytoplasm_Texture_AngularSecondMoment_Lipid_20_01 | 0.01 | 0.04 | 0.28 | 21.03 | 1.74 |
| Cytoplasm_Texture_AngularSecondMoment_Mito_10_01 | 0.00 | 0.04 | 0.39 | 35.12 | 2.27 |
| Cytoplasm_Texture_Correlation_Lipid_5_02 | 0.02 | 0.04 | 0.33 | 12.86 | −1.97 |
| Cytoplasm_Texture_Correlation_Mito_20_02 | 0.01 | 0.04 | 0.66 | 19.01 | 3.98 |
| Cytoplasm_Texture_DifferenceEntropy_Lipid_20_01 | 0.02 | 0.04 | 0.23 | 11.02 | −1.54 |
| Cytoplasm_Texture_DifferenceEntropy_DNA_5_00 | 0.02 | 0.04 | 0.38 | 10.93 | −2.22 |
| Cytoplasm_Texture_DifferenceEntropy_Mito_10_03 | 0.03 | 0.04 | 0.32 | 9.98 | −1.95 |
| Cytoplasm_Texture_DifferenceVariance_DNA_20_02 | 0.02 | 0.04 | 0.38 | 12.79 | 2.21 |
| Cytoplasm_Texture_DifferenceVariance_Mito_10_03 | 0.01 | 0.04 | 0.28 | 14.97 | 1.78 |
| Cytoplasm_Texture_Entropy_Lipid_10_01 | 0.01 | 0.04 | 0.25 | 14.36 | −1.62 |
| Cytoplasm_Texture_Entropy_Mito_5_01 | 0.02 | 0.04 | 0.32 | 12.10 | −1.93 |
| Cytoplasm_Texture_InfoMeas2_Lipid_20_00 | 0.01 | 0.04 | 0.33 | 21.95 | −1.98 |
| Cytoplasm_Texture_InfoMeas2_Mito_5_02 | 0.04 | 0.05 | 0.20 | 8.23 | −1.41 |
| Cytoplasm_Texture_InverseDifferenceMoment_Lipid_10_01 | 0.01 | 0.04 | 0.25 | 13.66 | 1.62 |
| Cytoplasm_Texture_InverseDifferenceMoment_DNA_5_01 | 0.02 | 0.04 | 0.32 | 10.42 | 1.95 |
| Cytoplasm_Texture_InverseDifferenceMoment_Mito_20_02 | 0.01 | 0.04 | 0.37 | 14.68 | 2.17 |
| Cytoplasm_Texture_SumAverage_Lipid_20_03 | 0.03 | 0.04 | 0.28 | 9.68 | −1.76 |
| Cytoplasm_Texture_SumAverage_Mito_20_01 | 0.03 | 0.05 | 0.29 | 8.47 | −1.79 |
| Cytoplasm_Texture_SumEntropy_Lipid_5_00 | 0.01 | 0.04 | 0.26 | 18.18 | −1.67 |
| Cytoplasm_Texture_SumEntropy_Mito_5_02 | 0.02 | 0.04 | 0.31 | 10.97 | −1.88 |

TABLE 6
List of significant genes (total 512 genes tested, known to be involved in adipocyte
function) for HOMA-IR PRS (linear regression model adj. BMI, sex, age batch, significance
level FDR 10%). Gene ID, Ensembl gene identification number; Gene name, gene name;
p-value, p-value of linear regression; q-value, q-value of linear regression

| Gene ID | Gene name | p-value | q-value |
|---|---|---|---|
| ENSG00000162433 | AK4 | 6.7E−05 | 1.8E−02 |
| ENSG00000004455 | AK2 | 1.1E−03 | 5.1E−02 |
| ENSG00000084234 | APLP2 | 1.1E−03 | 5.1E−02 |
| ENSG00000116171 | SCP2 | 9.4E−04 | 5.1E−02 |
| ENSG00000150593 | PDCD4 | 8.4E−04 | 5.1E−02 |
| ENSG00000165092 | ALDH1A1 | 9.1E−04 | 5.1E−02 |
| ENSG00000074800 | ENO1 | 4.4E−03 | 7.8E−02 |
| ENSG00000104812 | GYS1 | 5.5E−03 | 7.8E−02 |
| ENSG00000141526 | SLC16A3 | 2.2E−03 | 7.8E−02 |
| ENSG00000151640 | DPYSL4 | 4.4E−03 | 7.8E−02 |
| ENSG00000111275 | ALDH2 | 4.2E−03 | 7.8E−02 |
| ENSG00000122644 | ARL4A | 5.2E−03 | 7.8E−02 |
| ENSG00000130304 | SLC27A1 | 2.5E−03 | 7.8E−02 |
| ENSG00000141232 | TOB1 | 5.6E−03 | 7.8E−02 |
| ENSG00000148175 | STOM | 3.6E−03 | 7.8E−02 |
| ENSG00000151552 | QDPR | 4.7E−03 | 7.8E−02 |
| ENSG00000164237 | CMBL | 5.5E−03 | 7.8E−02 |
| ENSG00000211445 | GPX3 | 3.7E−03 | 7.8E−02 |
| ENSG00000159231 | CBR3 | 3.9E−03 | 7.8E−02 |
| ENSG00000188994 | ZNF292 | 6.5E−03 | 8.7E−02 |
| ENSG00000067057 | PFKP | 7.3E−03 | 9.2E−02 |
| ENSG00000105976 | MET | 8.0E−03 | 9.2E−02 |
| ENSG00000111669 | TPI1 | 8.3E−03 | 9.2E−02 |
| ENSG00000163516 | ANKZF1 | 8.0E−03 | 9.2E−02 |
| ENSG00000015532 | XYLT2 | 1.7E−02 | 9.5E−02 |
| ENSG00000102144 | PGK 1.00 | 1.7E−02 | 9.5E−02 |
| ENSG00000112715 | VEGFA | 1.1E−02 | 9.5E−02 |
| ENSG00000128039 | SRD5A3 | 1.1E−02 | 9.5E−02 |
| ENSG00000131724 | IL13RA1 | 1.7E−02 | 9.5E−02 |
| ENSG00000143590 | EFNA3 | 1.5E−02 | 9.5E−02 |
| ENSG00000143847 | PPFIA4 | 1.7E−02 | 9.5E−02 |
| ENSG00000146242 | TPBG | 9.8E−03 | 9.5E−02 |
| ENSG00000147852 | VLDLR | 1.7E−02 | 9.5E−02 |
| ENSG00000152952 | PLOD2 | 1.1E−02 | 9.5E−02 |
| ENSG00000167772 | ANGPTL4 | 1.6E−02 | 9.5E−02 |
| ENSG00000078070 | MCCC1 | 1.7E−02 | 9.5E−02 |
| ENSG00000127083 | OMD | 1.7E−02 | 9.5E−02 |
| ENSG00000143198 | MGST3 | 1.4E−02 | 9.5E−02 |
| ENSG00000152137 | HSPB8 | 1.2E−02 | 9.5E−02 |
| ENSG00000152583 | SPARCL1 | 1.3E−02 | 9.5E−02 |
| ENSG00000156709 | AIFM1 | 9.3E−03 | 9.5E−02 |
| ENSG00000205726 | ITSN1 | 1.2E−02 | 9.5E−02 |
| ENSG00000213619 | NDUFS3 | 9.2E−03 | 9.5E−02 |
| ENSG00000060971 | ACAA1 | 1.6E−02 | 9.5E−02 |
| ENSG00000100823 | APEX1 | 1.6E−02 | 9.5E−02 |
| ENSG00000104267 | CA2 | 1.3E−02 | 9.5E−02 |
| ENSG00000111897 | SERINC1 | 1.2E−02 | 9.5E−02 |
| ENSG00000159228 | CBR1 | 1.2E−02 | 9.5E−02 |
| ENSG00000248144 | ADH1C | 1.5E−02 | 9.5E−02 |
| ENSG00000117620 | SLC35A3 | 1.8E−02 | 9.5E−02 |
| ENSG00000130203 | APOE | 1.8E−02 | 9.5E−02 |

TABLE 7
List of KEGG pathways enriched among significantly associated genes with HOMA-IR polygenic risk.
KEGG-Pathway 2019

| Term | Overlap | p-value | adj. | OR | CS | Genes |
|---|---|---|---|---|---|---|
| Glycolysis/Gluconeogenesis | 5/68 | 8.1E−07 | 7.3E−05 | 3.4E+01 | 4.8E+02 | TPI1; ALDH2; ADH1C; ENO1; PFKP |
| Metabolism of xenobiotics by cytochrome P450 | 4/74 | 3.8E−05 | 1.1E−03 | 2.4E+01 | 2.5E+02 | CBR1; ADH1C; MGST3; CBR3 |
| PPAR signaling pathway | 4/74 | 3.8E−05 | 1.1E−03 | 2.4E+01 | 2.5E+02 | SLC27A1; SCP2; ANGPTL4; ACAA1 |
| Fatty acid degradation | 3/44 | 1.9E−04 | 4.3E−03 | 3.0E+01 | 2.6E+02 | ADH1C; ALDH2; ACAA1 |
| Valine, leucine and isoleucine degradation | 3/48 | 2.5E−04 | 4.5E−03 | 2.8E+01 | 2.3E+02 | ALDH2; MCCC1; ACAA1 |
| Arachidonic acid metabolism | 3/63 | 5.6E−04 | 7.4E−03 | 2.1E+01 | 1.6E+02 | CBR1; GPX3; CBR3 |
| Central carbon metabolism in cancer | 3/65 | 6.1E−04 | 7.4E−03 | 2.0E+01 | 1.5E+02 | SLC16A3; MET; PFKP |
| Thiamine metabolism | 2/15 | 6.6E−04 | 7.4E−03 | 6.3E+01 | 4.6E+02 | AK2; AK4 |
| RNA degradation | 3/79 | 1.1E−03 | 1.1E−02 | 1.6E+01 | 1.1E+02 | ENO1; TOB1; PFKP |
| Chemical carcinogenesis | 3/82 | 1.2E−03 | 1.1E−02 | 1.6E+01 | 1.1E+02 | CBR1; ADH1C; MGST3 |
| Folate biosynthesis | 2/26 | 2.0E−03 | 1.6E−02 | 3.4E+01 | 2.1E+02 | QDPR; CBR1 |
| Biosynthesis of unsaturated fatty acids | 2/27 | 2.1E−03 | 1.6E−02 | 3.3E+01 | 2.0E+02 | SCP2; ACAA1 |
| Fructose and mannose metabolism | 2/33 | 3.2E−03 | 2.2E−02 | 2.6E+01 | 1.5E+02 | TPI1; PFKP |
| MicroRNAs in cancer | 4/299 | 7.0E−03 | 4.3E−02 | 5.7E+00 | 2.8E+01 | EFNA3; PDCD4; MET; VEGFA |
| Cholesterol metabolism | 2/50 | 7.2E−03 | 4.3E−02 | 1.7E+01 | 8.3E+01 | ANGPTL4; APOE |
| Glutathione metabolism | 2/56 | 9.0E−03 | 5.1E−02 | 1.5E+01 | 7.1E+01 | GPX3; MGST3 |
| Lysine degradation | 2/59 | 9.9E−03 | 5.3E−02 | 1.4E+01 | 6.6E+01 | ALDH2; PLOD2 |
| PI3K-Akt signaling pathway | 4/354 | 1.3E−02 | 6.0E−02 | 4.8E+00 | 2.1E+01 | GYS1; EFNA3; MET; VEGFA |
| Retinol metabolism | 2/67 | 1.3E−02 | 6.0E−02 | 1.2E+01 | 5.5E+01 | ADH1C; ALDH1A1 |
| Renal cell carcinoma | 2/69 | 1.3E−02 | 6.0E−02 | 1.2E+01 | 5.2E+01 | MET; VEGFA |
| Proteoglycans in cancer | 3/201 | 1.5E−02 | 6.3E−02 | 6.2E+00 | 2.6E+01 | PDCD4; MET; VEGFA |
| Rap1 signaling pathway | 3/206 | 1.6E−02 | 6.4E−02 | 6.1E+00 | 2.5E+01 | EFNA3; MET; VEGFA |
| Peroxisome | 2/83 | 1.9E−02 | 7.4E−02 | 1.0E+01 | 4.0E+01 | SCP2; ACAA1 |
| Ras signaling pathway | 3/232 | 2.1E−02 | 8.0E−02 | 5.4E+00 | 2.1E+01 | EFNA3; MET; VEGFA |
| HIF-1 signaling pathway | 2/100 | 2.7E−02 | 9.7E−02 | 8.3E+00 | 3.0E+01 | ENO1; VEGFA |
| Insulin resistance | 2/108 | 3.1E−02 | 1.0E−01 | 7.6E+00 | 2.7E+01 | GYS1; SLC27A1 |
| Drug metabolism | 2/108 | 3.1E−02 | 1.0E−01 | 7.6E+00 | 2.7E+01 | ADH1C; MGST3 |
| AMPK signaling pathway | 2/120 | 3.8E−02 | 1.2E−01 | 6.9E+00 | 2.3E+01 | GYS1; PFKP |
| MAPK signaling pathway | 3/295 | 3.9E−02 | 1.2E−01 | 4.2E+00 | 1.4E+01 | EFNA3; MET; VEGFA |
| Nitrogen metabolism | 1/17 | 4.2E−02 | 1.2E−01 | 2.5E+01 | 7.9E+01 | CA2 |
| Primary bile acid biosynthesis | 1/17 | 4.2E−02 | 1.2E−01 | 2.5E+01 | 7.9E+01 | SCP2 |
| Purine metabolism | 2/129 | 4.3E−02 | 1.2E−01 | 6.4E+00 | 2.0E+01 | AK2; AK4 |
| Pathways in cancer | 4/530 | 4.6E−02 | 1.2E−01 | 3.1E+00 | 9.7E+00 | MGST3; MET; IL13RA1; VEGFA |
| Fluid shear stress and atherosclerosis | 2/139 | 4.9E−02 | 1.3E−01 | 5.9E+00 | 1.8E+01 | MGST3; VEGFA |
| Proximal tubule bicarbonate reclamation | 1/23 | 5.7E−02 | 1.4E−01 | 1.8E+01 | 5.2E+01 | CA2 |
| Histidine metabolism | 1/23 | 5.7E−02 | 1.4E−01 | 1.8E+01 | 5.2E+01 | ALDH2 |
| alpha-Linolenic acid metabolism | 1/25 | 6.2E−02 | 1.5E−01 | 1.7E+01 | 4.6E+01 | ACAA1 |
| Ascorbate and aldarate metabolism | 1/27 | 6.7E−02 | 1.5E−01 | 1.5E+01 | 4.2E+01 | ALDH2 |
| Collecting duct acid secretion | 1/27 | 6.7E−02 | 1.5E−01 | 1.5E+01 | 4.2E+01 | CA2 |
| Hepatocellular carcinoma | 2/168 | 6.8E−02 | 1.5E−01 | 4.9E+00 | 1.3E+01 | MGST3; MET |
| Alzheimer disease | 2/171 | 7.1E−02 | 1.5E−01 | 4.8E+00 | 1.3E+01 | NDUFS3; APOE |
| Pentose phosphate pathway | 1/30 | 7.4E−02 | 1.6E−01 | 1.4E+01 | 3.6E+01 | PFKP |
| beta-Alanine metabolism | 1/31 | 7.6E−02 | 1.6E−01 | 1.3E+01 | 3.4E+01 | ALDH2 |
| Galactose metabolism | 1/31 | 7.6E−02 | 1.6E−01 | 1.3E+01 | 3.4E+01 | PFKP |
| Axon guidance | 2/181 | 7.8E−02 | 1.6E−01 | 4.5E+00 | 1.2E+01 | EFNA3; MET |
| Base excision repair | 1/33 | 8.1E−02 | 1.6E−01 | 1.2E+01 | 3.1E+01 | APEX1 |
| Starch and sucrose metabolism | 1/36 | 8.8E−02 | 1.6E−01 | 1.1E+01 | 2.8E+01 | GYS1 |
| Tyrosine metabolism | 1/36 | 8.8E−02 | 1.6E−01 | 1.1E+01 | 2.8E+01 | ADH1C |
| Focal adhesion | 2/199 | 9.2E−02 | 1.7E−01 | 4.1E+00 | 9.8E+00 | MET; VEGFA |
| Pyruvate metabolism | 1/39 | 9.5E−02 | 1.7E−01 | 1.0E+01 | 2.5E+01 | ALDH2 |
| Bladder cancer | 1/41 | 9.9E−02 | 1.8E−01 | 1.0E+01 | 2.3E+01 | VEGFA |
| Tryptophan metabolism | 1/42 | 1.0E−01 | 1.8E−01 | 9.7E+00 | 2.2E+01 | ALDH2 |
| Arginine and proline metabolism | 1/49 | 1.2E−01 | 2.0E−01 | 8.3E+00 | 1.8E+01 | ALDH2 |
| Malaria | 1/49 | 1.2E−01 | 2.0E−01 | 8.3E+00 | 1.8E+01 | MET |
| N-Glycan biosynthesis | 1/50 | 1.2E−01 | 2.0E−01 | 8.1E+00 | 1.7E+01 | SRD5A3 |
| Glycosaminoglycan biosynthesis | 1/53 | 1.3E−01 | 2.0E−01 | 7.7E+00 | 1.6E+01 | XYLT2 |
| VEGF signaling pathway | 1/59 | 1.4E−01 | 2.2E−01 | 6.9E+00 | 1.3E+01 | VEGFA |
| Steroid hormone biosynthesis | 1/60 | 1.4E−01 | 2.2E−01 | 6.7E+00 | 1.3E+01 | SRD5A3 |
| Glycerolipid metabolism | 1/61 | 1.4E−01 | 2.2E−01 | 6.6E+00 | 1.3E+01 | ALDH2 |
| Epithelial cell signaling in Helicobacter pylori | 1/68 | 1.6E−01 | 2.3E−01 | 5.9E+00 | 1.1E+01 | MET |
| infection | | | | | | |
| Adherens junction | 1/72 | 1.7E−01 | 2.3E−01 | 5.6E+00 | 1.0E+01 | MET |
| Melanoma | 1/72 | 1.7E−01 | 2.3E−01 | 5.6E+00 | 1.0E+01 | MET |
| Bile secretion | 1/72 | 1.7E−01 | 2.3E−01 | 5.6E+00 | 1.0E+01 | CA2 |
| Inositol phosphate metabolism | 1/74 | 1.7E−01 | 2.3E−01 | 5.4E+00 | 9.6E+00 | TPI1 |
| Thyroid hormone synthesis | 1/74 | 1.7E−01 | 2.3E−01 | 5.4E+00 | 9.6E+00 | GPX3 |
| Bacterial invasion of epithelial cells | 1/74 | 1.7E−01 | 2.3E−01 | 5.4E+00 | 9.6E+00 | MET |
| Pancreatic cancer | 1/75 | 1.7E−01 | 2.3E−01 | 5.4E+00 | 9.4E+00 | VEGFA |
| Gastric acid secretion | 1/75 | 1.7E−01 | 2.3E−01 | 5.4E+00 | 9.4E+00 | CA2 |
| Rheumatoid arthritis | 1/91 | 2.1E−01 | 2.7E−01 | 4.4E+00 | 6.9E+00 | VEGFA |
| Pancreatic secretion | 1/98 | 2.2E−01 | 2.9E−01 | 4.1E+00 | 6.2E+00 | CA2 |
| AGE-RAGE signaling pathway in diabetic | 1/100 | 2.3E−01 | 2.9E−01 | 4.0E+00 | 6.0E+00 | VEGFA |
| complications | | | | | | |
| Glucagon signaling pathway | 1/103 | 2.3E−01 | 2.9E−01 | 3.9E+00 | 5.7E+00 | GYS1 |
| Thyroid hormone signaling pathway | 1/116 | 2.6E−01 | 3.2E−01 | 3.4E+00 | 4.7E+00 | PFKP |
| Relaxin signaling pathway | 1/130 | 2.8E−01 | 3.4E−01 | 3.1E+00 | 3.9E+00 | VEGFA |
| Oxidative phosphorylation | 1/133 | 2.9E−01 | 3.5E−01 | 3.0E+00 | 3.7E+00 | NDUFS3 |
| Insulin signaling pathway | 1/137 | 3.0E−01 | 3.5E−01 | 2.9E+00 | 3.5E+00 | GYS1 |
| Parkinson disease | 1/142 | 3.1E−01 | 3.5E−01 | 2.8E+00 | 3.3E+00 | NDUFS3 |
| Apoptosis | 1/143 | 3.1E−01 | 3.5E−01 | 2.8E+00 | 3.3E+00 | AIFM1 |
| Retrograde endocannabinoid signaling | 1/148 | 3.2E−01 | 3.5E−01 | 2.7E+00 | 3.1E+00 | NDUFS3 |
| Non-alcoholic fatty liver disease (NAFLD) | 1/149 | 3.2E−01 | 3.5E−01 | 2.7E+00 | 3.1E+00 | NDUFS3 |
| Gastric cancer | 1/149 | 3.2E−01 | 3.5E−01 | 2.7E+00 | 3.1E+00 | MET |
| JAK-STAT signaling pathway | 1/162 | 3.4E−01 | 3.7E−01 | 2.5E+00 | 2.7E+00 | IL13RA1 |
| Necroptosis | 1/162 | 3.4E−01 | 3.7E−01 | 2.5E+00 | 2.7E+00 | AIFM1 |
| Kaposi sarcoma-associated herpesvirus infection | 1/186 | 3.8E−01 | 4.0E−01 | 2.1E+00 | 2.1E+00 | VEGFA |
| Transcriptional misregulation in cancer | 1/186 | 3.8E−01 | 4.0E−01 | 2.1E+00 | 2.1E+00 | MET |
| Huntington disease | 1/193 | 3.9E−01 | 4.1E−01 | 2.1E+00 | 1.9E+00 | NDUFS3 |
| Human cytomegalovirus infection | 1/225 | 4.4E−01 | 4.5E−01 | 1.8E+00 | 1.5E+00 | VEGFA |
| Thermogenesis | 1/231 | 4.5E−01 | 4.6E−01 | 1.7E+00 | 1.4E+00 | NDUFS3 |
| Cytokine-cytokine receptor interaction | 1/294 | 5.3E−01 | 5.4E−01 | 1.3E+00 | 8.5E−01 | IL13RA1 |
| Human papillomavirus infection | 1/330 | 5.7E−01 | 5.7E−01 | 1.2E+00 | 6.7E−01 | VEGFA |

Term, which pathway; Overlap, number of genes that overlap and total genes; P-value, enrichment p-value; Adjusted P-value, Q-value; Odds Ratio, enrichment; Combined Score, approximation of overall association (−log10(P) * log(Odds)), Genes, genes in the pathway which are associated with HOMA-IR PRS

TABLE 8
Significant polygenic effects on LP profiles for lipodystrophy-like phenotype in
terminally differentiated subcutaneous and visceral AMSCs. (linear regression model adj.
BMI, sex, age, batch, significance level 5%). P-value, p-value of linear regression; q-value,
q-value of linear regression, estimate, estimate of linear regression model.

| | | | |
|---|---|---|---|
| LipocyteProfiler features | pvalue | q-value | estimate |

subcutaneous AMSCs day14

| | | | |
|---|---|---|---|
| Cells_Correlation_Correlation_DNA_AGP | 0.0039 | 0.0393 | −47.30 |
| Cells_Correlation_Correlation_Mito_AGP | 0.0013 | 0.0299 | −64.05 |
| Cells_Correlation_Overlap_Mito_AGP | 0.0020 | 0.0330 | −33.75 |
| Cells_Granularity_14_AGP | 0.0019 | 0.0330 | 6.32 |
| Cells_Granularity_14_Lipid | 0.0036 | 0.0393 | 2.14 |
| Cells_Granularity_15_AGP | 0.0011 | 0.0299 | 12.43 |
| Cells_Granularity_15_Mito | 0.0063 | 0.0409 | 12.24 |
| Cells_Granularity_16_AGP | 0.0064 | 0.0409 | 12.59 |
| Cells_Granularity_16_Lipid | 0.0042 | 0.0393 | −1.55 |
| Cells_Granularity_7_AGP | 0.0052 | 0.0393 | −11.80 |
| Cells_Intensity_IntegratedIntensity_Mito | 0.0063 | 0.0409 | 29.51 |
| Cells_Intensity_IntegratedIntensityEdge_Lipid | 0.0121 | 0.0467 | 2.15 |
| Cells_Intensity_IntegratedIntensityEdge_DNA | 0.0078 | 0.0433 | 27.05 |
| Cells_Intensity_IntegratedIntensityEdge_Mito | 0.0009 | 0.0293 | 50.58 |
| Cells_Intensity_LowerQuartileIntensity_Lipid | 0.0048 | 0.0393 | 5.65 |
| Cells_Intensity_LowerQuartileIntensity_Mito | 0.0008 | 0.0271 | 38.10 |
| Cells_Intensity_MassDisplacement_Lipid | 0.0129 | 0.0480 | −10.06 |
| Cells_Intensity_MaxIntensityEdge_Mito | 0.0050 | 0.0393 | 36.28 |
| Cells_Intensity_MeanIntensity_Mito | 0.0075 | 0.0433 | 28.08 |
| Cells_Intensity_MeanIntensityEdge_DNA | 0.0074 | 0.0433 | 15.09 |
| Cells_Intensity_MeanIntensityEdge_Mito | 0.0008 | 0.0271 | 44.58 |
| Cells_Intensity_MedianIntensity_Mito | 0.0041 | 0.0393 | 25.75 |
| Cells_Intensity_MinIntensity_Mito | 0.0038 | 0.0393 | 29.43 |
| Cells_Intensity_MinIntensityEdge_Mito | 0.0028 | 0.0368 | 30.60 |
| Cells_Intensity_StdIntensityEdge_Mito | 0.0086 | 0.0433 | 28.68 |
| Cells_Mean_LargeLipidObjects_Correlation_Correlation_DNA_AGP | 0.0117 | 0.0467 | −23.71 |
| Cells_Mean_LargeLipidObjects_Correlation_Correlation_Mito_AGP | 0.0099 | 0.0433 | −33.92 |
| Cells_Neighbors_PercentTouching_10 | 0.0112 | 0.0455 | 28.35 |
| Cells_Neighbors_PercentTouching_Adjacent | 0.0034 | 0.0393 | 23.72 |
| Cells_RadialDistribution_FracAtD_AGP_2of4 | 0.0112 | 0.0455 | −16.34 |
| Cells_RadialDistribution_FracAtD_AGP_4of4 | 0.0091 | 0.0433 | 19.39 |
| Cells_RadialDistribution_FracAtD_Lipid_4of4 | 0.0079 | 0.0433 | 20.69 |
| Cells_RadialDistribution_MeanFrac_AGP_3of4 | 0.0057 | 0.0406 | −12.30 |
| Cells_RadialDistribution_MeanFrac_AGP_4of4 | 0.0061 | 0.0409 | 19.50 |
| Cells_RadialDistribution_MeanFrac_Lipid_3of4 | 0.0122 | 0.0467 | −19.11 |
| Cells_RadialDistribution_MeanFrac_Lipid_4of4 | 0.0103 | 0.0441 | 19.84 |
| Cells_RadialDistribution_MeanFrac_Mito_3of4 | 0.0134 | 0.0491 | −13.26 |
| Cells_RadialDistribution_RadialCV_Lipid_4of4 | 0.0053 | 0.0393 | −40.29 |
| Cells_Texture_Contrast_Mito_5_03 | 0.0095 | 0.0433 | 48.68 |
| Cells_Texture_Correlation_AGP_5_03 | 0.0037 | 0.0393 | −40.89 |
| Cells_Texture_InfoMeas1_Mito_20_01 | 0.0001 | 0.0115 | −27.70 |
| Cells_Texture_InfoMeas2_AGP_5_02 | 0.0118 | 0.0467 | −40.71 |
| Cells_Texture_InfoMeas2_Mito_20_00 | 0.0012 | 0.0299 | 22.90 |
| Cells_Texture_SumAverage_Mito_5_02 | 0.0089 | 0.0433 | 27.13 |
| Cytoplasm_Correlation_Correlation_DNA_AGP | 0.0028 | 0.0368 | −56.82 |
| Cytoplasm_Correlation_Correlation_Mito_AGP | 0.0012 | 0.0299 | −62.74 |
| Cytoplasm_Correlation_Overlap_Mito_AGP | 0.0040 | 0.0393 | −33.97 |
| Cytoplasm_Granularity_14_AGP | 0.0021 | 0.0330 | 6.49 |
| Cytoplasm_Granularity_14_Lipid | 0.0035 | 0.0393 | 2.37 |
| Cytoplasm_Granularity_15_AGP | 0.0012 | 0.0299 | 12.13 |
| Cytoplasm_Granularity_15_Mito | 0.0072 | 0.0433 | 11.90 |
| Cytoplasm_Granularity_16_AGP | 0.0068 | 0.0424 | 12.17 |
| Cytoplasm_Granularity_16_Lipid | 0.0038 | 0.0393 | −1.35 |
| Cytoplasm_Granularity_7_AGP | 0.0089 | 0.0433 | −9.04 |
| Cytoplasm_Intensity_IntegratedIntensity_Mito | 0.0044 | 0.0393 | 31.81 |
| Cytoplasm_Intensity_IntegratedIntensityEdge_DNA | 0.0059 | 0.0408 | 30.95 |
| Cytoplasm_Intensity_IntegratedIntensityEdge_Mito | 0.0023 | 0.0336 | 42.61 |
| Cytoplasm_Intensity_LowerQuartileIntensity_Lipid | 0.0045 | 0.0393 | 10.24 |
| Cytoplasm_Intensity_LowerQuartileIntensity_DNA | 0.0099 | 0.0433 | 22.46 |
| Cytoplasm_Intensity_LowerQuartileIntensity_Mito | 0.0007 | 0.0262 | 41.23 |
| Cytoplasm_Intensity_MassDisplacement_Lipid | 0.0017 | 0.0330 | −14.52 |
| Cytoplasm_Intensity_MeanIntensity_Mito | 0.0047 | 0.0393 | 32.24 |
| Cytoplasm_Intensity_MeanIntensityEdge_DNA | 0.0089 | 0.0433 | 12.29 |
| Cytoplasm_Intensity_MeanIntensityEdge_Mito | 0.0022 | 0.0335 | 37.67 |
| Cytoplasm_Intensity_MedianIntensity_Mito | 0.0027 | 0.0368 | 29.73 |
| Cytoplasm_Intensity_MinIntensity_Mito | 0.0038 | 0.0393 | 29.43 |
| Cytoplasm_Intensity_MinIntensityEdge_Mito | 0.0028 | 0.0368 | 30.61 |
| Cytoplasm_Intensity_UpperQuartileIntensity_Mito | 0.0099 | 0.0433 | 22.71 |
| Cytoplasm_RadialDistribution_MeanFrac_Mito_2of4 | 0.0127 | 0.0479 | −8.68 |
| Cytoplasm_RadialDistribution_MeanFrac_Mito_3of4 | 0.0039 | 0.0393 | −9.92 |
| Cytoplasm_RadialDistribution_MeanFrac_Mito_4of4 | 0.0061 | 0.0409 | 11.29 |
| Cytoplasm_Texture_Contrast_Mito_5_03 | 0.0050 | 0.0393 | 57.18 |
| Cytoplasm_Texture_Correlation_AGP_5_03 | 0.0130 | 0.0480 | −29.05 |
| Cytoplasm_Texture_InfoMeas1_Mito_20_00 | 0.0001 | 0.0115 | −28.24 |
| Cytoplasm_Texture_InfoMeas2_Mito_20_00 | 0.0004 | 0.0248 | 24.06 |
| Cytoplasm_Texture_SumAverage_Mito_5_00 | 0.0049 | 0.0393 | 31.98 |
| Cytoplasm_Texture_SumVariance_Mito_5_02 | 0.0098 | 0.0433 | 56.60 |
| Cytoplasm_Texture_Variance_Mito_5_02 | 0.0095 | 0.0433 | 56.87 |

visceral AMSCs

| | | | |
|---|---|---|---|
| Cells_Texture_InfoMeas2_Lipid_20_01 | 0.0004 | 0.0343 | −25.55 |
| Cytoplasm_Texture_InfoMeas2_Lipid_20_01 | 0.0004 | 0.0343 | −28.47 |

TABLE 9
Significant effects on LP profile for 2p23.3 lipodystrophy locus in visceral AMSCs at day3
and day14 of differentiation. (ANOVA adj. BMI, sex, age, batch, significance level 5% FDR).
P-value, p-value of ANOVA, q-value, q-value of ANOVA, FDR; eta_sq, eta square
of ANOVA, effect size; F value of ANOVA; t-statistics of t-test.

| | | | | | |
|---|---|---|---|---|---|
| LipocyteProfiler features | p-value | q-value | eta_sq | F value | t-statistics |

day 14

| | | | | | |
|---|---|---|---|---|---|
| Cells_Correlation_K_AGP_Lipid | 0.00 | 0.03 | 0.59 | 17.19 | 3.13 |
| Cells_Granularity_1_Lipid | 0.00 | 0.03 | 0.52 | 21.88 | −2.72 |
| Cells_Intensity_MaxIntensity_Lipid | 0.00 | 0.02 | 0.58 | 29.14 | 3.27 |
| Cells_Intensity_MeanIntensity_Lipid | 0.00 | 0.03 | 0.60 | 21.64 | 3.50 |
| Cells_Intensity_StdIntensity_Lipid | 0.00 | 0.01 | 0.69 | 38.33 | 4.08 |
| Cells_LargeLipidObjects_AreaShape_Area | 0.01 | 0.05 | 0.45 | 10.85 | 2.37 |
| Cells_Mean_LargeLipidObjects_Intensity_IntegratedIntensity_Lipid | 0.01 | 0.04 | 0.50 | 13.50 | 2.70 |
| Cells_Mean_LargeLipidObjects_Intensity_IntegratedIntensityEdge_Lipid | 0.01 | 0.04 | 0.47 | 13.13 | 2.29 |
| Cells_Mean_LargeLipidObjects_Intensity_LowerQuartileIntensity_Lipid | 0.01 | 0.04 | 0.44 | 11.83 | 2.11 |
| Cells_Mean_LargeLipidObjects_Intensity_MassDisplacement_Lipid | 0.01 | 0.04 | 0.47 | 12.17 | 1.86 |
| Cells_Mean_LargeLipidObjects_Intensity_MaxIntensityEdge_Lipid | 0.01 | 0.04 | 0.48 | 12.40 | 2.50 |
| Cells_Mean_LargeLipidObjects_Intensity_MeanIntensity_Lipid | 0.01 | 0.04 | 0.44 | 11.55 | 2.24 |
| Cells_Mean_LargeLipidObjects_Intensity_MeanIntensityEdge_Lipid | 0.01 | 0.04 | 0.45 | 12.12 | 2.12 |
| Cells_Mean_LargeLipidObjects_Intensity_MedianIntensity_Lipid | 0.01 | 0.04 | 0.46 | 12.19 | 2.31 |
| Cells_Mean_LargeLipidObjects_Intensity_UpperQuartileIntensity_Lipid | 0.01 | 0.04 | 0.45 | 11.49 | 2.31 |
| Cells_Neighbors_PercentTouching_Adjacent | 0.01 | 0.05 | 0.51 | 11.03 | 3.42 |
| Cells_RadialDistribution_RadialCV_AGP_3of4 | 0.01 | 0.04 | 0.45 | 11.67 | −2.92 |
| Cells_Texture_AngularSecondMoment_Lipid_20_01 | 0.00 | 0.03 | 0.64 | 21.23 | −3.02 |
| Cells_Texture_Contrast_Lipid_5_00 | 0.00 | 0.03 | 0.64 | 22.92 | 3.64 |
| Cells_Texture_Correlation_Lipid_10_03 | 0.00 | 0.03 | 0.35 | 21.94 | 2.80 |
| Cells_Texture_Correlation_Mito_10_02 | 0.01 | 0.05 | 0.37 | 10.95 | 2.55 |
| Cells_Texture_DifferenceEntropy_Lipid_20_01 | 0.00 | 0.02 | 0.67 | 26.81 | 3.10 |
| Cells_Texture_Entropy_Lipid_20_01 | 0.00 | 0.02 | 0.68 | 25.98 | 3.08 |
| Cells_Texture_InfoMeas2_Lipid_5_03 | 0.00 | 0.01 | 0.64 | 70.52 | 4.04 |
| Cells_Texture_InverseDifferenceMoment_Lipid_20_01 | 0.00 | 0.02 | 0.68 | 27.50 | −3.00 |
| Cells_Texture_SumAverage_Lipid_20_03 | 0.00 | 0.02 | 0.72 | 25.13 | 3.82 |
| Cells_Texture_SumEntropy_Lipid_20_00 | 0.00 | 0.02 | 0.68 | 27.59 | 3.16 |
| Cells_Texture_SumVariance_Lipid_20_01 | 0.01 | 0.03 | 0.62 | 14.27 | 3.62 |
| Cells_Texture_Variance_Lipid_20_01 | 0.01 | 0.03 | 0.61 | 14.50 | 3.53 |
| Cytoplasm_Correlation_K_AGP_Lipid | 0.00 | 0.03 | 0.60 | 17.38 | 3.12 |
| Cytoplasm_Correlation_Overlap_DNA_AGP | 0.00 | 0.03 | 0.58 | 15.06 | −3.90 |
| Cytoplasm_Correlation_Overlap_DNA_Lipid | 0.01 | 0.04 | 0.53 | 12.85 | −2.85 |
| Cytoplasm_Correlation_Overlap_DNA_Mito | 0.00 | 0.03 | 0.54 | 17.45 | −2.20 |
| Cytoplasm_Granularity_1_Lipid | 0.00 | 0.03 | 0.53 | 21.85 | −2.78 |
| Cytoplasm_Granularity_13_Lipid | 0.01 | 0.04 | 0.11 | 11.95 | 1.16 |
| Cytoplasm_Intensity_MaxIntensity_Lipid | 0.00 | 0.02 | 0.57 | 23.92 | 3.20 |
| Cytoplasm_Intensity_MaxIntensityEdge_Lipid | 0.00 | 0.02 | 0.65 | 27.14 | 3.57 |
| Cytoplasm_Intensity_MeanIntensity_Lipid | 0.00 | 0.03 | 0.60 | 19.40 | 3.58 |
| Cytoplasm_Intensity_StdIntensity_Lipid | 0.00 | 0.02 | 0.67 | 27.65 | 3.97 |
| Cytoplasm_Intensity_StdIntensityEdge_Lipid | 0.00 | 0.01 | 0.69 | 44.45 | 3.96 |
| Cytoplasm_Texture_AngularSecondMoment_Lipid_20_03 | 0.00 | 0.03 | 0.63 | 16.17 | −3.13 |
| Cytoplasm_Texture_Contrast_Lipid_5_00 | 0.00 | 0.03 | 0.65 | 20.78 | 3.72 |
| Cytoplasm_Texture_Correlation_Lipid_10_02 | 0.00 | 0.01 | 0.55 | 40.73 | 3.59 |
| Cytoplasm_Texture_Correlation_Mito_5_02 | 0.00 | 0.03 | 0.46 | 17.49 | 2.79 |
| Cytoplasm_Texture_DifferenceEntropy_Lipid_20_01 | 0.00 | 0.03 | 0.63 | 18.68 | 2.87 |
| Cytoplasm_Texture_Entropy_Lipid_20_03 | 0.00 | 0.03 | 0.65 | 19.51 | 2.94 |
| Cytoplasm_Texture_InfoMeas1_Lipid_20_03 | 0.00 | 0.03 | 0.53 | 15.04 | −2.95 |
| Cytoplasm_Texture_InfoMeas1_DNA_5_02 | 0.01 | 0.04 | 0.19 | 11.67 | −1.74 |
| Cytoplasm_Texture_InfoMeas2_Lipid_5_02 | 0.00 | 0.01 | 0.72 | 53.74 | 4.25 |
| Cytoplasm_Texture_InverseDifferenceMoment_Lipid_20_01 | 0.00 | 0.02 | 0.71 | 27.33 | −3.10 |
| Cytoplasm_Texture_SumAverage_Lipid_20_01 | 0.00 | 0.03 | 0.68 | 20.02 | 3.67 |
| Cytoplasm_Texture_Variance_Lipid_20_01 | 0.01 | 0.04 | 0.60 | 12.91 | 3.46 |
| Nuclei_Correlation_Correlation_Lipid_AGP | 0.00 | 0.03 | 0.51 | 14.93 | −3.97 |
| Nuclei_Correlation_K_AGP_Lipid | 0.01 | 0.03 | 0.35 | 14.43 | 1.90 |
| Nuclei_Correlation_K_Lipid_AGP | 0.00 | 0.03 | 0.48 | 15.72 | −2.54 |
| Nuclei_Granularity_1_Lipid | 0.01 | 0.04 | 0.56 | 14.01 | −2.86 |
| Nuclei_Granularity_1_DNA | 0.00 | 0.01 | 0.83 | 101.09 | −7.42 |
| Nuclei_Granularity_1_Mito | 0.00 | 0.03 | 0.39 | 16.77 | −2.04 |
| Nuclei_Granularity_2_Mito | 0.00 | 0.03 | 0.39 | 16.16 | −3.06 |
| Nuclei_Intensity_LowerQuartileIntensity_Lipid | 0.00 | 0.02 | 0.64 | 23.97 | 2.83 |
| Nuclei_Intensity_LowerQuartileIntensity_DNA | 0.00 | 0.03 | 0.42 | 15.81 | 2.89 |
| Nuclei_Intensity_MaxIntensity_Lipid | 0.00 | 0.03 | 0.53 | 14.77 | 2.59 |
| Nuclei_Intensity_MaxIntensityEdge_Lipid | 0.01 | 0.04 | 0.54 | 13.38 | 2.56 |
| Nuclei_Intensity_MeanIntensity_Lipid | 0.01 | 0.04 | 0.43 | 13.32 | 2.14 |
| Nuclei_Intensity_MeanIntensityEdge_Lipid | 0.01 | 0.04 | 0.55 | 12.76 | 2.41 |
| Nuclei_Intensity_MedianIntensity_Lipid | 0.01 | 0.04 | 0.41 | 11.97 | 1.98 |
| Nuclei_Intensity_MinIntensity_Lipid | 0.00 | 0.03 | 0.68 | 22.90 | 3.03 |
| Nuclei_Intensity_MinIntensityEdge_Lipid | 0.00 | 0.03 | 0.65 | 18.74 | 2.84 |
| Nuclei_Intensity_StdIntensity_Lipid | 0.01 | 0.04 | 0.40 | 11.69 | 2.05 |
| Nuclei_Intensity_StdIntensityEdge_Lipid | 0.01 | 0.04 | 0.56 | 13.63 | 2.56 |
| Nuclei_RadialDistribution_RadialCV_DNA_1of4 | 0.00 | 0.02 | 0.64 | 24.16 | −2.91 |
| Nuclei_RadialDistribution_RadialCV_DNA_2of4 | 0.00 | 0.03 | 0.68 | 17.59 | −3.62 |
| Nuclei_RadialDistribution_RadialCV_DNA_3of4 | 0.00 | 0.03 | 0.59 | 16.79 | −3.80 |
| Nuclei_RadialDistribution_RadialCV_Mito_1of4 | 0.00 | 0.03 | 0.64 | 18.29 | −4.82 |
| Nuclei_Texture_Correlation_AGP_20_03 | 0.00 | 0.03 | 0.35 | 18.01 | −2.51 |
| Nuclei_Texture_Correlation_Lipid_20_03 | 0.00 | 0.02 | 0.40 | 25.21 | −2.77 |
| Nuclei_Texture_Correlation_Mito_20_03 | 0.00 | 0.02 | 0.39 | 25.59 | −2.72 |
| Nuclei_Texture_SumAverage_Lipid_10_01 | 0.00 | 0.03 | 0.47 | 15.41 | 2.31 |

day 3

| | | | | | |
|---|---|---|---|---|---|
| Cells_Correlation_K_AGP_Mito | 0.00 | 0.03 | 0.41 | 15.06 | −3.21 |
| Cells_Correlation_K_Mito_AGP | 0.00 | 0.02 | 0.48 | 23.26 | 3.29 |
| Cells_Granularity_4_Lipid | 0.01 | 0.03 | 0.46 | 11.77 | −3.29 |
| Cells_Granularity_7_Mito | 0.02 | 0.05 | 0.34 | 7.90 | −2.77 |
| Cells_Intensity_MassDisplacement_Lipid | 0.01 | 0.03 | 0.23 | 12.91 | −1.86 |
| Cells_Intensity_MaxIntensity_Mito | 0.00 | 0.03 | 0.41 | 16.42 | −2.94 |
| Cells_Intensity_MeanIntensityEdge_DNA | 0.02 | 0.05 | 0.24 | 8.07 | 2.13 |
| Cells_Intensity_MinIntensity_DNA | 0.01 | 0.03 | 0.24 | 12.68 | 2.02 |
| Cells_Intensity_MinIntensityEdge_DNA | 0.01 | 0.03 | 0.24 | 12.46 | 2.01 |
| Cells_Intensity_StdIntensity_Mito | 0.00 | 0.02 | 0.47 | 17.26 | −3.47 |
| Cells_Intensity_UpperQuartileIntensity_Mito | 0.01 | 0.03 | 0.33 | 12.13 | −2.15 |
| Cells_Mean_LargeLipidObjects_Correlation_K_DNA_Mito | 0.02 | 0.05 | 0.41 | 8.88 | −2.75 |
| Cells_RadialDistribution_FracAtD_Lipid_3of4 | 0.02 | 0.04 | 0.39 | 8.63 | −2.94 |
| Cells_RadialDistribution_FracAtD_Lipid_4of4 | 0.02 | 0.05 | 0.41 | 8.40 | 3.15 |
| Cells_RadialDistribution_MeanFrac_Lipid_3of4 | 0.01 | 0.03 | 0.41 | 12.35 | −2.96 |
| Cells_RadialDistribution_MeanFrac_Lipid_4of4 | 0.01 | 0.04 | 0.42 | 10.25 | 3.16 |
| Cells_RadialDistribution_RadialCV_Lipid_2of4 | 0.00 | 0.03 | 0.31 | 15.61 | −2.22 |
| Cells_RadialDistribution_RadialCV_Lipid_3of4 | 0.00 | 0.02 | 0.41 | 18.99 | −2.95 |
| Cells_RadialDistribution_RadialCV_Lipid_4of4 | 0.02 | 0.04 | 0.43 | 9.51 | −2.85 |
| Cells_Texture_Contrast_Mito_5_03 | 0.00 | 0.03 | 0.52 | 15.55 | −3.82 |
| Cells_Texture_DifferenceEntropy_Mito_10_03 | 0.00 | 0.02 | 0.52 | 20.31 | −3.76 |
| Cells_Texture_DifferenceVariance_Mito_10_03 | 0.00 | 0.02 | 0.46 | 19.81 | 3.20 |
| Cells_Texture_Entropy_Mito_10_02 | 0.00 | 0.02 | 0.38 | 16.97 | −2.47 |
| Cells_Texture_InverseDifferenceMoment_Mito_10_02 | 0.01 | 0.04 | 0.38 | 9.63 | 2.36 |
| Cells_Texture_SumAverage_Mito_10_01 | 0.01 | 0.03 | 0.36 | 11.04 | −2.25 |
| Cells_Texture_SumEntropy_Mito_20_02 | 0.00 | 0.02 | 0.37 | 21.86 | −2.62 |
| Cells_Texture_SumVariance_Mito_5_02 | 0.01 | 0.03 | 0.47 | 14.22 | −3.49 |
| Cells_Texture_Variance_Mito_10_02 | 0.01 | 0.03 | 0.47 | 13.99 | −3.55 |
| Cytoplasm_Correlation_K_AGP_Mito | 0.00 | 0.02 | 0.43 | 19.62 | −3.19 |
| Cytoplasm_Correlation_K_Mito_AGP | 0.00 | 0.02 | 0.46 | 21.85 | 3.12 |
| Cytoplasm_Correlation_K_Mito_DNA | 0.02 | 0.04 | 0.32 | 9.25 | 2.64 |
| Cytoplasm_Granularity_7_Mito | 0.02 | 0.04 | 0.38 | 9.47 | −3.02 |
| Cytoplasm_Granularity_8_Mito | 0.02 | 0.05 | 0.26 | 7.90 | −2.25 |
| Cytoplasm_Intensity_MassDisplacement_Lipid | 0.00 | 0.02 | 0.32 | 19.78 | −2.32 |
| Cytoplasm_Intensity_MaxIntensity_Mito | 0.00 | 0.02 | 0.41 | 17.35 | −2.92 |
| Cytoplasm_Intensity_MaxIntensityEdge_Mito | 0.00 | 0.02 | 0.45 | 17.86 | −3.27 |
| Cytoplasm_Intensity_MinIntensity_DNA | 0.01 | 0.03 | 0.24 | 12.68 | 2.02 |
| Cytoplasm_Intensity_MinIntensityEdge_DNA | 0.01 | 0.03 | 0.24 | 12.46 | 2.01 |
| Cytoplasm_Intensity_StdIntensity_Mito | 0.00 | 0.02 | 0.47 | 21.68 | −3.41 |
| Cytoplasm_Intensity_StdIntensityEdge_Mito | 0.00 | 0.02 | 0.44 | 18.13 | −3.09 |
| Cytoplasm_RadialDistribution_RadialCV_Lipid_2of4 | 0.01 | 0.03 | 0.33 | 10.54 | −2.44 |
| Cytoplasm_RadialDistribution_RadialCV_Lipid_3of4 | 0.01 | 0.03 | 0.41 | 13.46 | −2.94 |
| Cytoplasm_RadialDistribution_RadialCV_Lipid_4of4 | 0.01 | 0.03 | 0.42 | 11.33 | −2.95 |
| Cytoplasm_RadialDistribution_RadialCV_Mito_1of4 | 0.02 | 0.05 | 0.38 | 8.17 | −2.66 |
| Cytoplasm_Texture_Contrast_Mito_5_03 | 0.00 | 0.02 | 0.52 | 16.84 | −3.68 |
| Cytoplasm_Texture_Correlation_Lipid_5_02 | 0.01 | 0.03 | 0.32 | 10.62 | −2.17 |
| Cytoplasm_Texture_DifferenceEntropy_Mito_10_02 | 0.00 | 0.02 | 0.49 | 18.51 | −3.33 |
| Cytoplasm_Texture_DifferenceVariance_Mito_10_02 | 0.00 | 0.03 | 0.42 | 15.09 | 2.83 |
| Cytoplasm_Texture_InfoMeas2_Mito_10_01 | 0.02 | 0.05 | 0.35 | 8.26 | −2.49 |
| Cytoplasm_Texture_SumEntropy_Mito_20_01 | 0.01 | 0.03 | 0.29 | 13.69 | −1.99 |
| Cytoplasm_Texture_SumVariance_Mito_5_02 | 0.00 | 0.02 | 0.47 | 20.46 | −3.38 |
| Cytoplasm_Texture_Variance_Mito_5_01 | 0.00 | 0.02 | 0.48 | 20.35 | −3.44 |
| Nuclei_Correlation_K_AGP_Mito | 0.01 | 0.03 | 0.41 | 12.80 | −3.18 |
| Nuclei_Correlation_K_Mito_AGP | 0.00 | 0.02 | 0.48 | 18.09 | 3.28 |
| Nuclei_Granularity_1_Lipid | 0.00 | 0.02 | 0.38 | 31.62 | 2.53 |
| Nuclei_Granularity_3_Lipid | 0.02 | 0.05 | 0.36 | 8.37 | −2.80 |
| Nuclei_Granularity_4_Lipid | 0.01 | 0.03 | 0.44 | 13.59 | −3.09 |
| Nuclei_Intensity_IntegratedIntensity_Mito | 0.01 | 0.03 | 0.25 | 12.97 | −1.85 |
| Nuclei_Intensity_IntegratedIntensityEdge_Mito | 0.01 | 0.03 | 0.28 | 12.91 | −2.06 |
| Nuclei_Intensity_LowerQuartileIntensity_Mito | 0.00 | 0.02 | 0.45 | 22.43 | −3.18 |
| Nuclei_Intensity_MADIntensity_Mito | 0.01 | 0.04 | 0.43 | 10.36 | −3.35 |
| Nuclei_Intensity_MaxIntensity_Mito | 0.00 | 0.03 | 0.43 | 16.01 | −3.18 |
| Nuclei_Intensity_MaxIntensityEdge_Mito | 0.00 | 0.02 | 0.45 | 17.61 | −3.28 |
| Nuclei_Intensity_MeanIntensity_Mito | 0.00 | 0.02 | 0.45 | 19.46 | −3.26 |
| Nuclei_Intensity_MeanIntensityEdge_Mito | 0.00 | 0.02 | 0.44 | 21.61 | −3.10 |
| Nuclei_Intensity_MedianIntensity_Mito | 0.00 | 0.02 | 0.45 | 19.36 | −3.29 |
| Nuclei_Intensity_MinIntensity_Mito | 0.01 | 0.03 | 0.29 | 11.92 | −2.00 |
| Nuclei_Intensity_MinIntensityEdge_Mito | 0.01 | 0.04 | 0.28 | 10.44 | −1.94 |
| Nuclei_Intensity_StdIntensity_Mito | 0.01 | 0.03 | 0.45 | 11.46 | −3.45 |
| Nuclei_Intensity_StdIntensityEdge_Mito | 0.01 | 0.03 | 0.47 | 13.39 | −3.62 |
| Nuclei_Intensity_UpperQuartileIntensity_Mito | 0.00 | 0.02 | 0.45 | 17.38 | −3.30 |
| Nuclei_RadialDistribution_FracAtD_DNA_3of4 | 0.02 | 0.04 | 0.46 | 9.28 | −3.13 |
| Nuclei_Texture_Contrast_Mito_10_03 | 0.01 | 0.04 | 0.45 | 9.75 | −3.58 |
| Nuclei_Texture_Correlation_DNA_10_01 | 0.02 | 0.04 | 0.29 | 9.37 | 2.27 |
| Nuclei_Texture_DifferenceEntropy_Mito_10_02 | 0.01 | 0.03 | 0.45 | 12.45 | −3.43 |
| Nuclei_Texture_DifferenceVariance_Mito_5_03 | 0.01 | 0.03 | 0.40 | 13.36 | 2.93 |
| Nuclei_Texture_Entropy_Mito_5_00 | 0.01 | 0.03 | 0.42 | 12.01 | −3.16 |
| Nuclei_Texture_InfoMeas2_Mito_10_03 | 0.02 | 0.05 | 0.46 | 8.15 | −3.32 |
| Nuclei_Texture_InverseDifferenceMoment_Mito_10_00 | 0.01 | 0.03 | 0.44 | 11.05 | 3.26 |
| Nuclei_Texture_SumAverage_Mito_5_02 | 0.00 | 0.02 | 0.45 | 19.09 | −3.27 |
| Nuclei_Texture_SumEntropy_Mito_5_00 | 0.01 | 0.03 | 0.42 | 11.20 | −3.11 |
| Nuclei_Texture_SumVariance_Mito_5_02 | 0.02 | 0.04 | 0.43 | 9.48 | −3.40 |
| Nuclei_Texture_Variance_Mito_10_03 | 0.01 | 0.04 | 0.44 | 9.57 | −3.46 |

TABLE 10
List of genes with significant co-expression (q-value < 0.001, abs(estimate) >0.1 and <10
to exclude very low expression and housekeeping genes) with COBLLI across cohort
of 30 differentiating primary human adipocyte lines. Estimate betas provided by linear
regression with adjustment for 10 expression TPM (log-transformed) principal components,
day of differentiation, depot source, and donor as covariates.

| estimate | std.error | statistic | p.value | transcript | q.value |
|---|---|---|---|---|---|
| 0.12353721 | 0.018963106 | 6.514608456 | 5.44E−10 | ACVR1B | 6.72E−08 |
| 0.278498625 | 0.035055717 | 7.9444567 | 1.23E−13 | ADGRF5 | 3.80E−11 |
| −0.345241484 | 0.051818119 | −6.662563048 | 2.39E−10 | AIF1 | 3.23E−08 |
| 0.232391883 | 0.037023498 | 6.276875319 | 1.99E−09 | AKTIP | 2.17E−07 |
| 0.445746999 | 0.075994816 | 5.865492142 | 1.75E−08 | ANGPT1 | 1.42E−06 |
| 0.172190086 | 0.029272175 | 5.882380988 | 1.61E−08 | ANKRD40 | 1.32E−06 |
| 0.605316806 | 0.094489565 | 6.406176206 | 9.87E−10 | ANO6 | 1.16E−07 |
| 0.663393312 | 0.096709387 | 6.859657911 | 7.83E−11 | APBB2 | 1.25E−08 |
| 0.163500105 | 0.0272217 | 6.006241476 | 8.42E−09 | APPBP2 | 7.69E−07 |
| 0.148211492 | 0.025520873 | 5.807461673 | 2.36E−08 | AQP7P1 | 1.85E−06 |
| 0.392886492 | 0.032232707 | 12.18906303 | 4.09E−26 | ARHGEF6 | 4.58E−22 |
| 0.544845773 | 0.070844929 | 7.690681288 | 5.83E−13 | ARHGEF7 | 1.50E−10 |
| 0.314779245 | 0.053630715 | 5.869383706 | 1.72E−08 | ARPP19 | 1.40E−06 |
| 0.125477851 | 0.018609013 | 6.74285377 | 1.52E−10 | ATF7 | 2.18E−08 |
| 0.304335531 | 0.038649046 | 7.874334893 | 1.90E−13 | ATP11B | 5.54E−11 |
| 0.374462527 | 0.063314627 | 5.914313115 | 1.36E−08 | BCL2L13 | 1.15E−06 |
| 0.300020849 | 0.049777913 | 6.027188176 | 7.54E−09 | BNIP2 | 7.06E−07 |
| 3.233712959 | 0.450843133 | 7.172590024 | 1.28E−11 | BTNL9 | 2.40E−09 |
| 0.348218267 | 0.056221325 | 6.193704358 | 3.12E−09 | CA4 | 3.20E−07 |
| 0.267746305 | 0.040264475 | 6.649690718 | 2.56E−10 | CAB39L | 3.43E−08 |
| 0.180048985 | 0.019311342 | 9.323483884 | 1.75E−17 | CCDC170 | 1.62E−14 |
| 0.52589353 | 0.071392916 | 7.366186445 | 4.10E−12 | CD300LG | 8.56E−10 |
| −0.14693283 | 0.024458036 | −6.007548152 | 8.36E−09 | CD37 | 7.66E−07 |
| −0.287958114 | 0.036635887 | −7.86000107 | 2.07E−13 | CD53 | 5.95E−11 |
| 0.77292624 | 0.115116034 | 6.71432303 | 1.78E−10 | CDV3 | 2.51E−08 |
| 0.250508061 | 0.043384305 | 5.774163273 | 2.80E−08 | CHST3 | 2.14E−06 |
| 1 | 2.23E−17 | 44863746389628360 | 0 | COBLL1 | 0 |
| 0.216833474 | 0.033240249 | 6.523220547 | 5.18E−10 | COMMD2 | 6.52E−08 |
| 0.145515077 | 0.023737769 | 6.130107615 | 4.38E−09 | CRHBP | 4.33E−07 |
| 0.234961088 | 0.037738267 | 6.226069878 | 2.62E−09 | CRK | 2.76E−07 |
| 3.033158688 | 0.331400841 | 9.152537688 | 5.42E−17 | CSDE1 | 3.88E−14 |
| 0.166432488 | 0.026194456 | 6.353729573 | 1.31E−09 | CTNNBIP1 | 1.49E−07 |
| 0.208975566 | 0.029654582 | 7.046990854 | 2.67E−11 | DAPKZ | 4.67E−09 |
| 0.211103837 | 0.031356494 | 6.732380219 | 1.61E−10 | DLG1 | 2.30E−08 |
| 0.107373423 | 0.011311225 | 9.492643639 | 5.67E−18 | DMRT2 | 8.67E−15 |
| 0.108021003 | 0.018683073 | 5.781757919 | 2.70E−08 | DOP1A | 2.07E−06 |
| 0.161632436 | 0.024024311 | 6.727869855 | 1.65E−10 | DSG2 | 2.34E−08 |
| 0.212389257 | 0.031264483 | 6.793307833 | 1.14E−10 | DTD2 | 1.71E−08 |
| 0.546006767 | 0.072912966 | 7.488472868 | 1.98E−12 | DYNC1LI2 | 4.52E−10 |
| 0.473267525 | 0.079604183 | 5.945259506 | 1.16E−08 | ECE1 | 1.00E−06 |
| 0.175209767 | 0.027464867 | 6.379414428 | 1.14E−09 | EFCAB14 | 1.32E−07 |
| 0.18438036 | 0.027845952 | 6.62144224 | 3.00E−10 | EOGT | 3.96E−08 |
| 0.246789239 | 0.019728273 | 12.50941909 | 4.13E−27 | EPB41L4B | 6.94E−23 |
| 0.382348854 | 0.051355598 | 7.445125185 | 2.56E−12 | ESYT1 | 5.66E−10 |
| 0.168156447 | 0.028004252 | 6.004675474 | 8.49E−09 | ETV3 | 7.71E−07 |
| 0.215704432 | 0.029527808 | 7.305128625 | 5.89E−12 | FAM13B | 1.20E−09 |
| 0.1933708 | 0.031728937 | 6.094461996 | 5.29E−09 | FAM160B1 | 5.11E−07 |
| 0.22890165 | 0.028599561 | 8.003676965 | 8.53E−14 | FBXW2 | 2.84E−11 |
| −0.416143039 | 0.066798529 | −6.22982344 | 2.57E−09 | FCER1G | 2.71E−07 |
| 2.140486334 | 0.231555977 | 9.243926097 | 2.97E−17 | FERMT2 | 2.27E−14 |
| 0.138335158 | 0.02007716 | 6.89017553 | 6.58E−11 | FGF13 | 1.07E−08 |
| 0.105183946 | 0.015447457 | 6.80914327 | 1.04E−10 | FGFR2 | 1.57E−08 |
| 0.250670613 | 0.041064224 | 6.104355276 | 5.02E−09 | FMO3 | 4.89E−07 |
| 0.128802709 | 0.017594036 | 7.320816303 | 5.37E−12 | FOXN2 | 1.11E−09 |
| 0.634291963 | 0.104315855 | 6.080494337 | 5.69E−09 | GCLC | 5.45E−07 |
| 0.217669004 | 0.033961255 | 6.409333308 | 9.70E−10 | GDPD5 | 1.15E−07 |
| 0.135605273 | 0.017819721 | 7.609842667 | 9.52E−13 | GJA4 | 2.32E−10 |
| 0.332130737 | 0.04847028 | 6.852255331 | 8.17E−11 | GLYAT | 1.28E−08 |
| 0.903660764 | 0.094606321 | 9.551801111 | 3.82E−18 | GNAI1 | 6.75E−15 |
| 0.300257953 | 0.051780409 | 5.798678706 | 2.47E−08 | GPIHBP1 | 1.92E−06 |
| 0.249673499 | 0.042747371 | 5.840674994 | 1.99E−08 | HEATR5A | 1.59E−06 |
| 0.105319819 | 0.015076098 | 6.985880737 | 3.80E−11 | HEPACAM | 6.48E−09 |
| 0.235234107 | 0.030790468 | 7.639835344 | 7.94E−13 | HIPK3 | 1.99E−10 |
| 0.134027176 | 0.022561109 | 5.940628925 | 1.19E−08 | HPS5 | 1.02E−06 |
| 0.122582611 | 0.017933545 | 6.835380744 | 8.99E−11 | HSD17B13 | 1.39E−08 |
| 0.453141815 | 0.065677411 | 6.899507846 | 6.24E−11 | ID4 | 1.02E−08 |
| 0.162538978 | 0.016148272 | 10.06541 | 1.18E−19 | IGHV3-7 | 3.98E−16 |
| 0.310644517 | 0.053402853 | 5.817002267 | 2.25E−08 | IRS2 | 1.77E−06 |
| 0.419852203 | 0.051949808 | 8.081881646 | 5.25E−14 | ITGA1 | 1.88E−11 |
| −0.192356063 | 0.032479697 | −5.922347774 | 1.31E−08 | ITGAM | 1.11E−06 |
| 3.423504735 | 0.584584126 | 5.856308073 | 1.84E−08 | ITIH5 | 1.48E−06 |
| 0.279405495 | 0.048400209 | 5.772815845 | 2.82E−08 | ITPK1 | 2.15E−06 |
| 1.237137166 | 0.129268123 | 9.570318931 | 3.37E−18 | ITSN1 | 6.30E−15 |
| 0.296318808 | 0.035620049 | 8.318877059 | 1.18E−14 | KANK1 | 5.04E−12 |
| 0.117361909 | 0.017357296 | 6.761531891 | 1.37E−10 | KCTD18 | 2.00E−08 |
| 0.357544939 | 0.060472889 | 5.912483199 | 1.37E−08 | KCTD20 | 1.15E−06 |
| 0.231797583 | 0.036659637 | 6.322964465 | 1.55E−09 | KIAA1109 | 1.73E−07 |
| 0.216806946 | 0.03043285 | 7.124109104 | 1.70E−11 | LATS2 | 3.06E−09 |
| 3.966395581 | 0.480902936 | 8.24780904 | 1.86E−14 | LEP | 7.42E−12 |
| 0.498703237 | 0.063460208 | 7.858518728 | 2.09E−13 | LIMCH1 | 5.96E−11 |
| 0.260033734 | 0.042902463 | 6.061044368 | 6.31E−09 | LIMS2 | 5.99E−07 |
| 0.814166708 | 0.119370882 | 6.820479938 | 9.79E−11 | LMOD1 | 1.49E−08 |
| 0.330827265 | 0.055280552 | 5.984514474 | 9.44E−09 | LNPEP | 8.43E−07 |
| 0.1593283 | 0.026839332 | 5.936373454 | 1.21E−08 | LPGAT1 | 1.04E−06 |
| 0.410648338 | 0.064610691 | 6.355733549 | 1.30E−09 | LRP5 | 1.49E−07 |
| 0.307980734 | 0.044421099 | 6.933208366 | 5.14E−11 | MAP4K3 | 8.55E−09 |
| 0.952879508 | 0.136087283 | 7.001973177 | 3.46E−11 | MBNL1 | 5.96E−09 |
| 0.122035062 | 0.016711543 | 7.302441212 | 5.98E−12 | MBNL3 | 1.21E−09 |
| 0.501798633 | 0.075861884 | 6.614634498 | 3.12E−10 | MSRB3 | 4.05E−08 |
| 0.603688428 | 0.083305002 | 7.246724845 | 8.31E−12 | MTIF3 | 1.61E−09 |
| 0.162918021 | 0.023757815 | 6.857449552 | 7.93E−11 | MTMR12 | 1.25E−08 |
| 0.831308682 | 0.143167114 | 5.806561719 | 2.37E−08 | MTURN | 1.85E−06 |
| 0.429335554 | 0.059172986 | 7.255600666 | 7.89E−12 | NBPF19 | 1.57E−09 |
| 2.4104225 | 0.330537222 | 7.292438905 | 6.35E−12 | NFE2L1 | 1.28E−09 |
| 0.707341736 | 0.062157528 | 11.37982417 | 1.28E−23 | NPY1R | 8.61E−20 |
| 0.23988759 | 0.036471236 | 6.577446099 | 3.84E−10 | NRIP1 | 4.90E−08 |
| 0.578547544 | 0.091854738 | 6.298505225 | 1.77E−09 | OSBPL9 | 1.95E−07 |
| 0.289195726 | 0.045321626 | 6.380965367 | 1.13E−09 | OSGIN2 | 1.31E−07 |
| 0.11899215 | 0.013230451 | 8.993808894 | 1.54E−16 | OTUD7B | 1.03E−13 |
| 0.394357387 | 0.06140338 | 6.42240522 | 9.03E−10 | PAIP1 | 1.08E−07 |
| 0.979084729 | 0.167815716 | 5.834285093 | 2.06E−08 | PALMD | 1.64E−06 |
| 0.121262333 | 0.018159266 | 6.677711119 | 2.19E−10 | PARM1 | 3.03E−08 |
| 0.29988266 | 0.041871089 | 7.162045817 | 1.37E−11 | PBRM1 | 2.54E−09 |
| 0.613651216 | 0.086911273 | 7.060663058 | 2.46E−11 | PEAK1 | 4.36E−09 |
| 1.367753044 | 0.226106507 | 6.049153834 | 6.72E−09 | PICALM | 6.34E−07 |
| 0.203693388 | 0.031672101 | 6.43131904 | 8.60E−10 | PIK3CA | 1.03E−07 |
| 7.441090414 | 1.235249224 | 6.023958786 | 7.67E−09 | PLIN1 | 7.16E−07 |
| 0.193736659 | 0.028396477 | 6.822559575 | 9.67E−11 | POLI | 1.48E−08 |
| 0.220177524 | 0.036380795 | 6.052026146 | 6.62E−09 | PPM1A | 6.26E−07 |
| 2.622061326 | 0.328846241 | 7.97351771 | 1.03E−13 | PPP1R14A | 3.29E−11 |
| 0.183990649 | 0.029808035 | 6.172518641 | 3.49E−09 | PTBP2 | 3.54E−07 |
| 0.517720804 | 0.084060073 | 6.158938289 | 3.75E−09 | PTGER3 | 3.76E−07 |
| 0.172366256 | 0.022607414 | 7.624324185 | 8.72E−13 | PTPN21 | 2.16E−10 |
| 0.514551942 | 0.066191926 | 7.773636034 | 3.52E−13 | PTPRM | 9.54E−11 |
| 2.777613616 | 0.355083747 | 7.822418333 | 2.61E−13 | PXDN | 7.25E−11 |
| 0.210662803 | 0.035705624 | 5.899989466 | 1.47E−08 | RANBP9 | 1.22E−06 |
| 0.280903257 | 0.046656004 | 6.020731209 | 7.80E−09 | RBM28 | 7.24E−07 |
| 1.76529664 | 0.262223106 | 6.732040762 | 1.61E−10 | RDH10 | 2.30E−08 |
| 0.297656949 | 0.046978064 | 6.336083826 | 1.45E−09 | RHOBTB1 | 1.63E−07 |
| 0.661062671 | 0.067090101 | 9.853356376 | 5.01E−19 | RIMKLB | 1.12E−15 |
| 0.389558552 | 0.064681431 | 6.022726271 | 7.72E−09 | RNF41 | 7.19E−07 |
| 0.681479621 | 0.11462057 | 5.94552636 | 1.16E−08 | ROCK2 | 1.00E−06 |
| 0.225587295 | 0.033824944 | 6.669258566 | 2.30E−10 | SAMD8 | 3.14E−08 |
| 0.899873296 | 0.12984221 | 6.930514345 | 5.22E−11 | SBDS | 8.64E−09 |
| 0.496386584 | 0.07433563 | 6.677640094 | 2.19E−10 | SBDSP1 | 3.03E−08 |
| 0.113019481 | 0.017988529 | 6.282863886 | 1.93E−09 | SCN4A | 2.11E−07 |
| 0.163132376 | 0.019391905 | 8.412395354 | 6.55E−15 | SCTR | 3.06E−12 |
| 0.580328303 | 0.078430257 | 7.399291097 | 3.37E−12 | SEL1L | 7.21E−10 |
| 1.807970931 | 0.250330714 | 7.222329627 | 9.59E−12 | SEPTIN11 | 1.83E−09 |
| 0.146817211 | 0.02053113 | 7.150956331 | 1.46E−11 | SEPTIN4 | 2.66E−09 |
| 1.465272751 | 0.225156808 | 6.507787901 | 5.65E−10 | SERINC1 | 6.95E−08 |
| 0.338346773 | 0.045355017 | 7.459963602 | 2.35E−12 | SIAH1 | 5.22E−10 |
| 0.104960872 | 0.018154119 | 5.781655944 | 2.70E−08 | SIX1 | 2.07E−06 |
| 1.244822049 | 0.122608306 | 10.15283623 | 6.51E−20 | SLC19A3 | 2.74E−16 |
| 0.19423283 | 0.024337342 | 7.980856455 | 9.83E−14 | SLC31A2 | 3.21E−11 |
| 0.55630051 | 0.090466872 | 6.149217893 | 3.95E−09 | SLC44A2 | 3.92E−07 |
| 0.215057323 | 0.027027456 | 7.956994796 | 1.14E−13 | SNRK | 3.55E−11 |
| 0.147805189 | 0.024186221 | 6.111131941 | 4.84E−09 | SPIRE1 | 4.73E−07 |
| 2.422807191 | 0.313056793 | 7.739193802 | 4.34E−13 | SPTBN1 | 1.15E−10 |
| 0.465966949 | 0.072836744 | 6.397415983 | 1.04E−09 | STX12 | 1.21E−07 |
| 0.570405778 | 0.067445955 | 8.457227447 | 4.92E−15 | STX7 | 2.33E−12 |
| 0.706744203 | 0.093810435 | 7.533748306 | 1.51E−12 | SYNPO2 | 3.52E−10 |
| 0.180132315 | 0.020497852 | 8.787863042 | 5.88E−16 | TBC1D9 | 3.35E−13 |
| 0.804403375 | 0.075860951 | 10.60365526 | 2.92E−21 | TCF7L2 | 1.40E−17 |
| 0.360581425 | 0.043270476 | 8.333197608 | 1.08E−14 | TEAD1 | 4.79E−12 |
| 0.717383228 | 0.075524664 | 9.498661633 | 5.45E−18 | THBS4 | 8.67E−15 |
| 0.537535539 | 0.090031254 | 5.970543719 | 1.02E−08 | TINAGL1 | 8.98E−07 |
| 0.141007883 | 0.023965279 | 5.883840594 | 1.59E−08 | TJP1 | 1.31E−06 |
| 0.451549035 | 0.075045815 | 6.016978264 | 7.96E−09 | TNIP1 | 7.35E−07 |
| 2.800753974 | 0.283307648 | 9.885910237 | 4.01E−19 | TNS1 | 9.64E−16 |
| 0.481059905 | 0.053615879 | 8.972340256 | 1.77E−16 | TNS3 | 1.14E−13 |
| 0.133394488 | 0.022948716 | 5.812721144 | 2.30E−08 | TRAPPC6B | 1.80E−06 |
| 0.480782059 | 0.080353794 | 5.983314998 | 9.50E−09 | TRIP12 | 8.46E−07 |
| 0.699867971 | 0.078568141 | 8.907783223 | 2.70E−16 | TSPAN15 | 1.65E−13 |
| 0.15156668 | 0.022325318 | 6.789004349 | 1.17E−10 | TUFT1 | 1.73E−08 |
| 0.583304957 | 0.080449159 | 7.250603568 | 8.12E−12 | UGP2 | 1.59E−09 |
| 0.209906204 | 0.033642995 | 6.239224734 | 2.44E−09 | USP24 | 2.60E−07 |
| 0.262732586 | 0.042914618 | 6.122216574 | 4.56E−09 | UTP23 | 4.48E−07 |
| 0.815844534 | 0.133290844 | 6.120784508 | 4.60E−09 | UTRN | 4.51E−07 |
| 0.181844488 | 0.01674266 | 10.8611469 | 4.87E−22 | VIPR1 | 2.73E−18 |
| 0.232482598 | 0.030084412 | 7.727676438 | 4.66E−13 | WDR11 | 1.21E−10 |
| 0.242453172 | 0.036325758 | 6.674414699 | 2.23E−10 | WWC2 | 3.08E−08 |
| 0.227140895 | 0.035534451 | 6.392131811 | 1.07E−09 | ZFYVE16 | 1.24E−07 |
| 0.173627658 | 0.028846975 | 6.018920786 | 7.88E−09 | ZMYM2 | 7.29E−07 |
| 0.104893301 | 0.018038542 | 5.814954449 | 2.27E−08 | ZRANB1 | 1.79E−06 |

TABLE 11
List of KEGG pathways enriched among PAC-coexpressed genes.

| | | | | | Old | | | |
|---|---|---|---|---|---|---|---|---|
| | | | Adjusted | Old P- | Adjusted | Odds | Combined | |
| Term | Overlap | P-value | P-value | value | P-value | Ratio | Score | Genes |
| Regulation of | 6/55 | 5.97E−06 | 0.001051128 | 0 | 0 | 15.23758183 | 183.2833576 | PIK3CA; PTGER3; NPY1R; |
| lipolysis in | | | | | | | | IRS2; PLIN1; GNAI1 |
| adipocytes | | | | | | | | |
| Regulation of | 8/214 | 4.11E−04 | 0.036204713 | 0 | 0 | 4.855358358 | 37.85190174 | ITGAM; PIK3CA; ROCK2; |
| actin | | | | | | | | ITGA1; ARHGEF7; CRK; |
| cytoskeleton | | | | | | | | FGFR2; ARHGEF6 |
| Rap1 signaling | 6/206 | 0.007341186 | 0.318496889 | 0 | 0 | 3.704716981 | 18.20592333 | ITGAM; PIK3CA; ANGPT1; |
| pathway | | | | | | | | CRK; FGFR2; GNAI1 |
| Phagosome | 5/152 | 0.008611652 | 0.318496889 | 0 | 0 | 4.18537415 | 19.89994374 | STX12; DYNC1LI2; ITGAM; |
| | | | | | | | | STX7; THBS4 |
| Wnt signaling | 5/158 | 0.010075751 | 0.318496889 | 0 | 0 | 4.02001634 | 18.48252214 | TCF7L2; ROCK2; CTNNBIP1; |
| pathway | | | | | | | | SIAH1; LRP5 |
| Leukocyte | 4/112 | 0.013933402 | 0.318496889 | 0 | 0 | 4.538072234 | 19.39329862 | ITGAM; PIK3CA; ROCK2; |
| transendothelial | | | | | | | | GNAI1 |
| migration | | | | | | | | |
| Sphingolipid | 4/119 | 0.017048192 | 0.318496889 | 0 | 0 | 4.260329463 | 17.34683097 | FCER1G; PIK3CA; ROCK2; |
| signaling | | | | | | | | GNAI1 |
| pathway | | | | | | | | |
| Acute myeloid | 3/66 | 0.017266234 | 0.318496889 | 0 | 0 | 5.811875367 | 23.59041656 | TCF7L2; ITGAM; PIK3CA |
| leukemia | | | | | | | | |
| AMPK signaling | 4/120 | 0.01752578 | 0.318496889 | 0 | 0 | 4.223388306 | 17.07973005 | PIK3CA; CAB39L; LEP; IRS2 |
| pathway | | | | | | | | |
| Platelet | 4/124 | 0.019519304 | 0.318496889 | 0 | 0 | 4.081780538 | 16.06732245 | FCER1G; PIK3CA; ROCK2; |
| activation | | | | | | | | GNAI1 |
| Adherens | 3/72 | 0.021715697 | 0.318496889 | 0 | 0 | 5.304884595 | 20.31622219 | TJP1; TCF7L2; PTPRM |
| junction | | | | | | | | |
| Arrhythmogenic | 3/72 | 0.021715697 | 0.318496889 | 0 | 0 | 5.304884595 | 20.31622219 | TCF7L2; ITGA1; DSG2 |
| right ventricular | | | | | | | | |
| cardiomyopathy | | | | | | | | |
| (ARVC) | | | | | | | | |
| Focal adhesion | 5/199 | 0.024804889 | 0.335820037 | 0 | 0 | 3.163820876 | 11.69574252 | PIK3CA; ROCK2; ITGA1; |
| | | | | | | | | CRK; THBS4 |
| Signaling | 4/139 | 0.028211489 | 0.343186531 | 0 | 0 | 3.625488843 | 12.93583833 | PIK3CA; ID4; ACVR1B; |
| pathways | | | | | | | | FGFR2 |
| regulating | | | | | | | | |
| pluripotency of | | | | | | | | |
| stem cells | | | | | | | | |
| CAMP signaling | 5/212 | 0.031398435 | 0.343186531 | 0 | 0 | 2.963164251 | 10.25550322 | PIK3CA; ROCK2; PTGER3; |
| pathway | | | | | | | | NPY1R; GNAI1 |
| Pathways in | 9/530 | 0.032015288 | 0.343186531 | 0 | 0 | 2.138712535 | 7.360468442 | TCF7L2; PIK3CA; ROCK2; |
| cancer | | | | | | | | DAPK2; PTGER3; LRP5; |
| | | | | | | | | CRK; FGFR2; GNAI1 |
| Salmonella | 3/86 | 0.03430246 | 0.343186531 | 0 | 0 | 4.406961178 | 14.86264501 | TJP1; DYNC1LI2; ROCK2 |
| infection | | | | | | | | |
| Gastric cancer | 4/149 | 0.035103139 | 0.343186531 | 0 | 0 | 3.373741701 | 11.30022879 | TCF7L2; PIK3CA; LRP5; |
| | | | | | | | | FGFR2 |
| TGF-beta | 3/90 | 0.038460102 | 0.343186531 | 0 | 0 | 4.203490847 | 13.69553595 | ZFYVE16; ID4; ACVR1B |
| signaling | | | | | | | | |
| pathway | | | | | | | | |
| Human | 5/225 | 0.038998469 | 0.343186531 | 0 | 0 | 2.786221591 | 9.039151696 | PIK3CA; ROCK2; PTGER3; |
| cytomegalovirus | | | | | | | | CRK; GNAI1 |
| infection | | | | | | | | |
| Hippo signaling | 4/160 | 0.043716765 | 0.354471625 | 0 | 0 | 3.134097786 | 9.809800074 | TCF7L2; DLG1; LATS2; |
| pathway | | | | | | | | TEAD1 |
| Hematopoietic | 3/97 | 0.046322996 | 0.354471625 | 0 | 0 | 3.889085894 | 11.94772595 | ITGAM; ITGA1; CD37 |
| cell lineage | | | | | | | | |
| Prostate cancer | 3/97 | 0.046322996 | 0.354471625 | 0 | 0 | 3.889085894 | 11.94772595 | TCF7L2; PIK3CA; FGFR2 |
| Hepatocellular | 4/168 | 0.050662859 | 0.369520809 | 0 | 0 | 2.98000303 | 8.888044387 | TCF7L2; PBRM1; PIK3CA; |
| carcinoma | | | | | | | | LRP5 |
| Tight junction | 4/170 | 0.052488751 | 0.369520809 | 0 | 0 | 2.943800045 | 8.675839123 | TJP1; EPB41L4B; DLG1; |
| | | | | | | | | ROCK2 |
| Type II diabetes | 2/46 | 0.055282054 | 0.374216984 | 0 | 0 | 5.518962632 | 15.97909079 | PIK3CA; IRS2 |
| mellitus | | | | | | | | |
| Chemokine | 4/190 | 0.072684137 | 0.473792891 | 0 | 0 | 2.62459093 | 6.880711887 | PIK3CA; ROCK2; CRK; |
| signaling | | | | | | | | GNAI1 |
| pathway | | | | | | | | |
| Legionellosis | 2/55 | 0.075640687 | 0.475455748 | 0 | 0 | 4.579696724 | 11.82368216 | BCL2L13; ITGAM |
| Endometrial | 2/58 | 0.082881874 | 0.481848918 | 0 | 0 | 4.33369851 | 10.79237792 | TCF7L2; PIK3CA |
| cancer | | | | | | | | |
| Autophagy | 3/128 | 0.089530683 | 0.481848918 | 0 | 0 | 2.92 | 7.046467757 | PIK3CA; DAPK2; IRS2 |
| MAPK signaling | 5/295 | 0.097814113 | 0.481848918 | 0 | 0 | 2.106142241 | 4.896120237 | PPM1A; ANGPT1; CRK; |
| pathway | | | | | | | | FGFR2; MAP4K3 |
| Central carbon | 2/65 | 0.100540737 | 0.481848918 | 0 | 0 | 3.850813127 | 8.846058235 | PIK3CA; FGFR2 |
| metabolism in | | | | | | | | |
| cancer | | | | | | | | |
| Shigellosis | 2/65 | 0.100540737 | 0.481848918 | 0 | 0 | 3.850813127 | 8.846058235 | ROCK2; CRK |
| Ubiquitin | 3/137 | 0.104380233 | 0.481848918 | 0 | 0 | 2.722636816 | 6.152383143 | SIAH1; TRIP12; RHOBTB1 |
| mediated | | | | | | | | |
| proteolysis | | | | | | | | |
| Insulin signaling | 3/137 | 0.104380233 | 0.481848918 | 0 | 0 | 2.722636816 | 6.152383143 | PIK3CA; IRS2; CRK |
| pathway | | | | | | | | |
| Fc epsilon RI | 2/68 | 0.10840254 | 0.481848918 | 0 | 0 | 3.675218442 | 8.165981683 | FCER1G; PIK3CA |
| signaling | | | | | | | | |
| pathway | | | | | | | | |
| Adipocytokine | 2/69 | 0.111058735 | 0.481848918 | 0 | 0 | 3.620181302 | 7.956058228 | LEP; IRS2 |
| signaling | | | | | | | | |
| pathway | | | | | | | | |
| Renal cell | 2/69 | 0.111058735 | 0.481848918 | 0 | 0 | 3.620181302 | 7.956058228 | PIK3CA; CRK |
| carcinoma | | | | | | | | |
| Breast cancer | 3/147 | 0.121929162 | 0.481848918 | 0 | 0 | 2.532278807 | 5.328712393 | TCF7L2; PIK3CA; LRP5 |
| Bacterial | 2/74 | 0.124585955 | 0.481848918 | 0 | 0 | 3.367927744 | 7.014583174 | PIK3CA; CRK |
| invasion of | | | | | | | | |
| epithelial cells | | | | | | | | |
| Inositol | 2/74 | 0.124585955 | 0.481848918 | 0 | 0 | 3.367927744 | 7.014583174 | PIK3CA; ITPK1 |
| phosphate | | | | | | | | |
| metabolism | | | | | | | | |
| Non-alcoholic | 3/149 | 0.125561971 | 0.481848918 | 0 | 0 | 2.497336377 | 5.181862725 | PIK3CA; LEP; IRS2 |
| fatty liver | | | | | | | | |
| disease (NAFLD) | | | | | | | | |
| Pancreatic | 2/75 | 0.127337534 | 0.481848918 | 0 | 0 | 3.321623666 | 6.84558061 | PIK3CA; ARHGEF6 |
| cancer | | | | | | | | |
| Chronic myeloid | 2/76 | 0.130103483 | 0.481848918 | 0 | 0 | 3.27657105 | 6.682321303 | PIK3CA; CRK |
| leukemia | | | | | | | | |
| Pertussis | 2/76 | 0.130103483 | 0.481848918 | 0 | 0 | 3.27657105 | 6.682321303 | ITGAM; GNAI1 |
| mTOR signaling | 3/152 | 0.131083752 | 0.481848918 | 0 | 0 | 2.446681581 | 4.971458382 | PIK3CA; CAB39L; LRP5 |
| pathway | | | | | | | | |
| Nitrogen | 1/17 | 0.131413341 | 0.481848918 | 0 | 0 | 7.552972561 | 15.32806027 | CA4 |
| metabolism | | | | | | | | |
| Phenylalanine | 1/17 | 0.131413341 | 0.481848918 | 0 | 0 | 7.552972561 | 15.32806027 | GLYAT |
| metabolism | | | | | | | | |
| Human | 5/330 | 0.138042831 | 0.495827312 | 0 | 0 | 1.875961538 | 3.714762664 | TCF7L2; DLG1; PIK3CA; |
| papillomavirus | | | | | | | | ITGA1; THBS4 |
| infection | | | | | | | | |
| ECM-receptor | 2/82 | 0.146977805 | 0.509143066 | 0 | 0 | 3.029907975 | 5.80976882 | ITGA1; THBS4 |
| interaction | | | | | | | | |
| Neuroactive | 5/338 | 0.14813625 | 0.509143066 | 0 | 0 | 1.830142643 | 3.494882159 | VIPR1; LEP; PTGER3; |
| ligand-receptor | | | | | | | | NPY1R; SCTR |
| interaction | | | | | | | | |
| ErbB signaling | 2/85 | 0.155576201 | 0.509143066 | 0 | 0 | 2.919949738 | 5.432915793 | PIK3CA; CRK |
| pathway | | | | | | | | |
| cGMP-PKG | 3/166 | 0.157915901 | 0.509143066 | 0 | 0 | 2.234946603 | 4.125024538 | ROCK2; IRS2; GNAI1 |
| signaling | | | | | | | | |
| pathway | | | | | | | | |
| Colorectal | 2/86 | 0.158463673 | 0.509143066 | 0 | 0 | 2.885042361 | 5.314911319 | TCF7L2; PIK3CA |
| cancer | | | | | | | | |
| Gap junction | 2/88 | 0.164268429 | 0.509143066 | 0 | 0 | 2.817663005 | 5.089413453 | TJP1; GNAI1 |
| Other types of | 1/22 | 0.166689124 | 0.509143066 | 0 | 0 | 5.753193961 | 10.30756459 | EOGT |
| O-glycan | | | | | | | | |
| biosynthesis | | | | | | | | |
| PI3K-Akt | 5/354 | 0.169226681 | 0.509143066 | 0 | 0 | 1.74480659 | 3.099677091 | PIK3CA; ANGPT1; ITGA1; |
| signaling | | | | | | | | FGFR2; THBS4 |
| pathway | | | | | | | | |
| Fc gamma R- | 2/91 | 0.173044787 | 0.509143066 | 0 | 0 | 2.722272007 | 4.775422718 | PIK3CA; CRK |
| mediated | | | | | | | | |
| phagocytosis | | | | | | | | |
| Proximal tubule | 1/23 | 0.1735715 | 0.509143066 | 0 | 0 | 5.491407982 | 9.616365099 | CA4 |
| bicarbonate | | | | | | | | |
| reclamation | | | | | | | | |
| Renin- | 1/23 | 0.1735715 | 0.509143066 | 0 | 0 | 5.491407982 | 9.616365099 | LNPEP |
| angiotensin | | | | | | | | |
| system | | | | | | | | |
| Vitamin | 1/24 | 0.180397375 | 0.509873565 | 0 | 0 | 5.252386002 | 8.995200678 | SLC19A3 |
| digestion and | | | | | | | | |
| absorption | | | | | | | | |
| Amoebiasis | 2/96 | 0.187833957 | 0.509873565 | 0 | 0 | 2.576817648 | 4.308946524 | ITGAM; PIK3CA |
| Axon guidance | 3/181 | 0.188340387 | 0.509873565 | 0 | 0 | 2.045047857 | 3.414216364 | PIK3CA; ROCK2; GNAI1 |
| Choline | 2/99 | 0.196790329 | 0.509873565 | 0 | 0 | 2.496742774 | 4.058746088 | PIK3CA; SLC44A2 |
| metabolism in | | | | | | | | |
| cancer | | | | | | | | |
| Phosphatidylino | 2/99 | 0.196790329 | 0.509873565 | 0 | 0 | 2.496742774 | 4.058746088 | PIK3CA; ITPK1 |
| sitol signaling | | | | | | | | |
| system | | | | | | | | |
| Progesterone- | 2/99 | 0.196790329 | 0.509873565 | 0 | 0 | 2.496742774 | 4.058746088 | PIK3CA; GNAI1 |
| mediated | | | | | | | | |
| oocyte | | | | | | | | |
| maturation | | | | | | | | |
| HIF-1 signaling | 2/100 | 0.199787737 | 0.509873565 | 0 | 0 | 2.471140603 | 3.979771422 | PIK3CA; ANGPT1 |
| pathway | | | | | | | | |
| T cell receptor | 2/101 | 0.202790622 | 0.509873565 | 0 | 0 | 2.446055649 | 3.902880526 | DLG1; PIK3CA |
| signaling | | | | | | | | |
| pathway | | | | | | | | |
| Melanogenesis | 2/101 | 0.202790622 | 0.509873565 | 0 | 0 | 2.446055649 | 3.902880526 | TCF7L2; GNAI1 |
| Ribosome | 2/101 | 0.202790622 | 0.509873565 | 0 | 0 | 2.446055649 | 3.902880526 | RBM28; SBDS |
| biogenesis in | | | | | | | | |
| eukaryotes | | | | | | | | |
| Longevity | 2/102 | 0.205798693 | 0.510148873 | 0 | 0 | 2.421472393 | 3.828001115 | PIK3CA; IRS2 |
| regulating | | | | | | | | |
| pathway | | | | | | | | |
| Chagas disease | 2/103 | 0.208811661 | 0.510428504 | 0 | 0 | 2.397375934 | 3.755064053 | PIK3CA; GNAI1 |
| (American | | | | | | | | |
| trypanosomiasis) | | | | | | | | |
| C-type lectin | 2/104 | 0.211829243 | 0.510711599 | 0 | 0 | 2.373751955 | 3.684003185 | FCER1G; PIK3CA |
| receptor | | | | | | | | |
| signaling | | | | | | | | |
| pathway | | | | | | | | |
| Parathyroid | 2/106 | 0.217877137 | 0.518022284 | 0 | 0 | 2.327866918 | 3.547259399 | LRP5; GNAI1 |
| hormone | | | | | | | | |
| synthesis, | | | | | | | | |
| secretion and | | | | | | | | |
| action | | | | | | | | |
| Insulin | 2/108 | 0.223940194 | 0.518022284 | 0 | 0 | 2.283713393 | 3.417294489 | PIK3CA; IRS2 |
| resistance | | | | | | | | |
| Asthma | 1/31 | 0.226634749 | 0.518022284 | 0 | 0 | 4.025406504 | 5.975376181 | FCER1G |
| Galactose | 1/31 | 0.226634749 | 0.518022284 | 0 | 0 | 4.025406504 | 5.975376181 | UGP2 |
| metabolism | | | | | | | | |
| Cholinergic | 2/112 | 0.236103398 | 0.532746128 | 0 | 0 | 2.20022309 | 3.175990004 | PIK3CA; GNAI1 |
| synapse | | | | | | | | |
| Pentose and | 1/34 | 0.245648204 | 0.540426049 | 0 | 0 | 3.658906135 | 5.136573052 | UGP2 |
| glucuronate | | | | | | | | |
| interconversions | | | | | | | | |
| SNARE | 1/34 | 0.245648204 | 0.540426049 | 0 | 0 | 3.658906135 | 5.136573052 | STX7 |
| interactions in | | | | | | | | |
| vesicular | | | | | | | | |
| transport | | | | | | | | |
| Alanine, | 1/35 | 0.251882195 | 0.540682171 | 0 | 0 | 3.551111908 | 4.896251013 | RIMKLB |
| aspartate and | | | | | | | | |
| glutamate | | | | | | | | |
| metabolism | | | | | | | | |
| Human | 3/212 | 0.255111941 | 0.540682171 | 0 | 0 | 1.738968634 | 2.375523048 | PIK3CA; CRK; GNAI1 |
| immunodeficiency | | | | | | | | |
| virus 1 | | | | | | | | |
| infection | | | | | | | | |
| Neurotrophin | 2/119 | 0.257466687 | 0.540682171 | 0 | 0 | 2.067851712 | 2.805795483 | PIK3CA; CRK |
| signaling | | | | | | | | |
| pathway | | | | | | | | |
| Starch and | 1/36 | 0.258064978 | 0.540682171 | 0 | 0 | 3.449477352 | 4.672468415 | UGP2 |
| sucrose | | | | | | | | |
| metabolism | | | | | | | | |
| Aldosterone- | 1/37 | 0.26419697 | 0.540682171 | 0 | 0 | 3.35348916 | 4.463696472 | PIK3CA |
| regulated | | | | | | | | |
| sodium | | | | | | | | |
| reabsorption | | | | | | | | |
| Thyroid cancer | 1/37 | 0.26419697 | 0.540682171 | 0 | 0 | 3.35348916 | 4.463696472 | TCF7L2 |
| Ferroptosis | 1/40 | 0.282292336 | 0.566410991 | 0 | 0 | 3.095059412 | 3.914668568 | GCLC |
| Bladder cancer | 1/41 | 0.288225281 | 0.566410991 | 0 | 0 | 3.017530488 | 3.753846786 | DAPK2 |
| Relaxin signaling | 2/130 | 0.291073744 | 0.566410991 | 0 | 0 | 1.889091258 | 2.331476057 | PIK3CA; GNAI1 |
| pathway | | | | | | | | |
| Natural killer | 2/131 | 0.294123325 | 0.566410991 | 0 | 0 | 1.874352024 | 2.293749772 | FCER1G; PIK3CA |
| cell mediated | | | | | | | | |
| cytotoxicity | | | | | | | | |
| Vascular | 2/132 | 0.297171154 | 0.566410991 | 0 | 0 | 1.859839547 | 2.256816773 | PPP1R14A; ROCK2 |
| smooth muscle | | | | | | | | |
| contraction | | | | | | | | |
| FoxO signaling | 2/132 | 0.297171154 | 0.566410991 | 0 | 0 | 1.859839547 | 2.256816773 | PIK3CA; IRS2 |
| pathway | | | | | | | | |
| Ras signaling | 3/232 | 0.29980895 | 0.566410991 | 0 | 0 | 1.585476306 | 1.909880362 | PIK3CA; ANGPT1; FGFR2 |
| pathway | | | | | | | | |
| Vasopressin- | 1/44 | 0.305733205 | 0.566410991 | 0 | 0 | 2.806579694 | 3.325916035 | DYNC1LI2 |
| regulated water | | | | | | | | |
| reabsorption | | | | | | | | |
| Carbohydrate | 1/44 | 0.305733205 | 0.566410991 | 0 | 0 | 2.806579694 | 3.325916035 | PIK3CA |
| digestion and | | | | | | | | |
| absorption | | | | | | | | |
| Apelin signaling | 2/137 | 0.312377902 | 0.566788771 | 0 | 0 | 1.790502159 | 2.083323745 | PLIN1; GNAI1 |
| pathway | | | | | | | | |
| Estrogen | 2/137 | 0.312377902 | 0.566788771 | 0 | 0 | 1.790502159 | 2.083323745 | PIK3CA; GNAI1 |
| signaling | | | | | | | | |
| pathway | | | | | | | | |
| Cysteine and | 1/47 | 0.322813023 | 0.574496388 | 0 | 0 | 2.623144221 | 2.965941956 | GCLC |
| methionine | | | | | | | | |
| metabolism | | | | | | | | |
| Endocytosis | 3/244 | 0.326859053 | 0.574496388 | 0 | 0 | 1.505609344 | 1.683611864 | ZFYVE16; FGFR2; RNF41 |
| Amino sugar | 1/48 | 0.328412966 | 0.574496388 | 0 | 0 | 2.567202906 | 2.858537872 | UGP2 |
| and nucleotide | | | | | | | | |
| sugar | | | | | | | | |
| metabolism | | | | | | | | |
| Cocaine | 1/49 | 0.333966879 | 0.574496388 | 0 | 0 | 2.51359248 | 2.756690693 | GNAI1 |
| addiction | | | | | | | | |
| Malaria | 1/49 | 0.333966879 | 0.574496388 | 0 | 0 | 2.51359248 | 2.756690693 | THBS4 |
| Cell adhesion | 2/145 | 0.336556621 | 0.574496388 | 0 | 0 | 1.689647776 | 1.840007639 | ITGAM; PTPRM |
| molecules | | | | | | | | |
| (CAMs) | | | | | | | | |
| Vibrio cholerae | 1/50 | 0.339475139 | 0.574496388 | 0 | 0 | 2.462170234 | 2.66001685 | TJP1 |
| infection | | | | | | | | |
| Phospholipase D | 2/148 | 0.345562004 | 0.57922774 | 0 | 0 | 1.654676864 | 1.758231821 | FCER1G; PIK3CA |
| signaling | | | | | | | | |
| pathway | | | | | | | | |
| Glycosaminogly | 1/53 | 0.355729708 | 0.586264082 | 0 | 0 | 2.319770169 | 2.397677523 | CHST3 |
| can biosynthesis | | | | | | | | |
| Oxytocin | 2/153 | 0.36048152 | 0.586264082 | 0 | 0 | 1.59947995 | 1.631972723 | ROCK2; GNAI1 |
| signaling | | | | | | | | |
| pathway | | | | | | | | |
| Fanconi anemia | 1/54 | 0.361059052 | 0.586264082 | 0 | 0 | 2.275885872 | 2.318476245 | POLI |
| pathway | | | | | | | | |
| Pathogenic | 1/55 | 0.366344576 | 0.586264082 | 0 | 0 | 2.23362692 | 2.242965542 | ROCK2 |
| Escherichia coli | | | | | | | | |
| infection | | | | | | | | |
| Cushing | 2/155 | 0.366415051 | 0.586264082 | 0 | 0 | 1.578411324 | 1.584706924 | TCF7L2; GNAI1 |
| syndrome | | | | | | | | |
| Glutathione | 1/56 | 0.37158664 | 0.58918242 | 0 | 0 | 2.192904656 | 2.170916895 | GCLC |
| metabolism | | | | | | | | |
| Cellular | 2/160 | 0.381154913 | 0.597559529 | 0 | 0 | 1.528073309 | 1.47390218 | PIK3CA; HIPK3 |
| senescence | | | | | | | | |
| JAK-STAT | 2/162 | 0.387010951 | 0.597559529 | 0 | 0 | 1.508819018 | 1.43232535 | PIK3CA; LEP |
| signaling | | | | | | | | |
| pathway | | | | | | | | |
| VEGF signaling | 1/59 | 0.387055604 | 0.597559529 | 0 | 0 | 2.079163162 | 1.973514471 | PIK3CA |
| pathway | | | | | | | | |
| Long-term | 1/60 | 0.392127348 | 0.600125332 | 0 | 0 | 2.04381976 | 1.913359935 | GNAI1 |
| depression | | | | | | | | |
| Basal cell | 1/63 | 0.407093659 | 0.608623636 | 0 | 0 | 1.944630212 | 1.747662505 | TCF7L2 |
| carcinoma | | | | | | | | |
| Mitophagy | 1/65 | 0.416867143 | 0.608623636 | 0 | 0 | 1.88366997 | 1.648188073 | BCL2L13 |
| Non-small cell | 1/66 | 0.421693666 | 0.608623636 | 0 | 0 | 1.854596623 | 1.60139993 | PIK3CA |
| lung cancer | | | | | | | | |
| Retinol | 1/67 | 0.42648048 | 0.608623636 | 0 | 0 | 1.826404287 | 1.556441058 | RDH10 |
| metabolism | | | | | | | | |
| Staphylococcus | 1/68 | 0.431227911 | 0.608623636 | 0 | 0 | 1.799053513 | 1.513217252 | ITGAM |
| aureus infection | | | | | | | | |
| Epithelial cell | 1/68 | 0.431227911 | 0.608623636 | 0 | 0 | 1.799053513 | 1.513217252 | TJP1 |
| signaling in | | | | | | | | |
| Helicobacter | | | | | | | | |
| pylori infection | | | | | | | | |
| Tuberculosis | 2/179 | 0.435741226 | 0.608623636 | 0 | 0 | 1.362725729 | 1.132025434 | ITGAM; FCER1G |
| Renin secretion | 1/69 | 0.43593628 | 0.608623636 | 0 | 0 | 1.772507174 | 1.471640377 | GNAI1 |
| Prolactin | 1/70 | 0.440605906 | 0.608623636 | 0 | 0 | 1.746730293 | 1.431627903 | PIK3CA |
| signaling | | | | | | | | |
| pathway | | | | | | | | |
| B cell receptor | 1/71 | 0.445237107 | 0.608623636 | 0 | 0 | 1.721689895 | 1.393102475 | PIK3CA |
| signaling | | | | | | | | |
| pathway | | | | | | | | |
| MicroRNAs in | 3/299 | 0.449022173 | 0.608623636 | 0 | 0 | 1.22240991 | 0.978762845 | PIK3CA; IRS2; CRK |
| cancer | | | | | | | | |
| Bile secretion | 1/72 | 0.449830197 | 0.608623636 | 0 | 0 | 1.697354861 | 1.35599152 | SCTR |
| p53 signaling | 1/72 | 0.449830197 | 0.608623636 | 0 | 0 | 1.697354861 | 1.35599152 | SIAH1 |
| pathway | | | | | | | | |
| Melanoma | 1/72 | 0.449830197 | 0.608623636 | 0 | 0 | 1.697354861 | 1.35599152 | PIK3CA |
| Transcriptional | 2/186 | 0.455202647 | 0.608623636 | 0 | 0 | 1.310416111 | 1.031313965 | ITGAM; SIX1 |
| misregulation in | | | | | | | | |
| cancer | | | | | | | | |
| Leishmaniasis | 1/74 | 0.458903288 | 0.608623636 | 0 | 0 | 1.650684932 | 1.285744562 | ITGAM |
| PPAR signaling | 1/74 | 0.458903288 | 0.608623636 | 0 | 0 | 1.650684932 | 1.285744562 | PLIN1 |
| pathway | | | | | | | | |
| Gastric acid | 1/75 | 0.463383904 | 0.608623636 | 0 | 0 | 1.628295979 | 1.252484292 | GNAI1 |
| secretion | | | | | | | | |
| Glioma | 1/75 | 0.463383904 | 0.608623636 | 0 | 0 | 1.628295979 | 1.252484292 | PIK3CA |
| Complement | 1/79 | 0.480940582 | 0.627004018 | 0 | 0 | 1.544480926 | 1.130577871 | ITGAM |
| and coagulation | | | | | | | | |
| cascades | | | | | | | | |
| Viral | 2/201 | 0.495579736 | 0.636657179 | 0 | 0 | 1.210716157 | 0.849955454 | DLG1; PIK3CA |
| carcinogenesis | | | | | | | | |
| Proteoglycans in | 2/201 | 0.495579736 | 0.636657179 | 0 | 0 | 1.210716157 | 0.849955454 | PIK3CA; ROCK2 |
| cancer | | | | | | | | |
| Hypertrophic | 1/85 | 0.506210687 | 0.645602036 | 0 | 0 | 1.433725319 | 0.976083522 | ITGA1 |
| cardiomyopathy | | | | | | | | |
| (HCM) | | | | | | | | |
| GABAergic | 1/89 | 0.522374192 | 0.657220715 | 0 | 0 | 1.368278825 | 0.888520732 | GNAI1 |
| synapse | | | | | | | | |
| Dilated | 1/91 | 0.530257622 | 0.657220715 | 0 | 0 | 1.337737127 | 0.848650148 | ITGA1 |
| cardiomyopathy | | | | | | | | |
| (DCM) | | | | | | | | |
| Morphine | 1/91 | 0.530257622 | 0.657220715 | 0 | 0 | 1.337737127 | 0.848650148 | GNAI1 |
| addiction | | | | | | | | |
| Rheumatoid | 1/91 | 0.530257622 | 0.657220715 | 0 | 0 | 1.337737127 | 0.848650148 | ANGPT1 |
| arthritis | | | | | | | | |
| Small cell lung | 1/93 | 0.538011708 | 0.66179526 | 0 | 0 | 1.30852333 | 0.811120844 | PIK3CA |
| cancer | | | | | | | | |
| Human T-cell | 2/219 | 0.541468849 | 0.66179526 | 0 | 0 | 1.109270306 | 0.680503768 | DLG1; PIK3CA |
| leukemia virus 1 | | | | | | | | |
| infection | | | | | | | | |
| Circadian | 1/97 | 0.553140249 | 0.666393369 | 0 | 0 | 1.253747459 | 0.742398653 | GNAI1 |
| entrainment | | | | | | | | |
| Glycerophospho | 1/97 | 0.553140249 | 0.666393369 | 0 | 0 | 1.253747459 | 0.742398653 | LPGAT1 |
| lipid | | | | | | | | |
| metabolism | | | | | | | | |
| Pancreatic | 1/98 | 0.556844799 | 0.666393369 | 0 | 0 | 1.240759366 | 0.726425792 | SCTR |
| secretion | | | | | | | | |
| AGE-RAGE | 1/100 | 0.564162568 | 0.666393369 | 0 | 0 | 1.215570338 | 0.695808054 | PIK3CA |
| signaling | | | | | | | | |
| pathway in | | | | | | | | |
| diabetic | | | | | | | | |
| complications | | | | | | | | |
| Inflammatory | 1/100 | 0.564162568 | 0.666393369 | 0 | 0 | 1.215570338 | 0.695808054 | PIK3CA |
| mediator | | | | | | | | |
| regulation of | | | | | | | | |
| TRP channels | | | | | | | | |
| Toll-like | 1/104 | 0.578439713 | 0.678702597 | 0 | 0 | 1.168126924 | 0.639457151 | PIK3CA |
| receptor | | | | | | | | |
| signaling | | | | | | | | |
| pathway | | | | | | | | |
| Drug | 1/108 | 0.592251908 | 0.69030686 | 0 | 0 | 1.124230682 | 0.588898128 | FMO3 |
| metabolism | | | | | | | | |
| TNF signaling | 1/110 | 0.598988373 | 0.693565484 | 0 | 0 | 1.103490714 | 0.565553438 | PIK3CA |
| pathway | | | | | | | | |
| Toxoplasmosis | 1/113 | 0.608886201 | 0.69506512 | 0 | 0 | 1.073769599 | 0.532722751 | GNAI1 |
| Serotonergic | 1/113 | 0.608886201 | 0.69506512 | 0 | 0 | 1.073769599 | 0.532722751 | GNAI1 |
| synapse | | | | | | | | |
| Glutamatergic | 1/114 | 0.612131214 | 0.69506512 | 0 | 0 | 1.064213253 | 0.522325036 | GNAI1 |
| synapse | | | | | | | | |
| Thyroid | 1/116 | | | 0 | 0 | | | |
| hormone | | | | | | | | |
| signaling | | 0.618541173 | 0.697841324 | | | 1.045599152 | 0.502296966 | PIK3CA |
| pathway | | | | | | | | |
| Osteoclast | 1/127 | 0.651959155 | 0.73085867 | 0 | 0 | 0.953784359 | 0.408003545 | PIK3CA |
| differentiation | | | | | | | | |
| Dopaminergic | 1/131 | 0.663375549 | 0.738949979 | 0 | 0 | 0.924249531 | 0.379324956 | GNAI1 |
| synapse | | | | | | | | |
| Measles | 1/138 | 0.682465702 | 0.751853115 | 0 | 0 | 0.876713548 | 0.33494228 | PIK3CA |
| Fluid shear | 1/139 | 0.685103549 | 0.751853115 | 0 | 0 | 0.870316366 | 0.329140843 | PIK3CA |
| stress and | | | | | | | | |
| atherosclerosis | | | | | | | | |
| Parkinson | 1/142 | 0.692887116 | 0.751853115 | 0 | 0 | 0.851669261 | 0.31246739 | GNAI1 |
| disease | | | | | | | | |
| Apoptosis | 1/143 | 0.695438903 | 0.751853115 | 0 | 0 | 0.84562865 | 0.307142573 | PIK3CA |
| Adrenergic | 1/145 | 0.700479425 | 0.751853115 | 0 | 0 | 0.833799119 | 0.296824387 | GNAI1 |
| signaling in | | | | | | | | |
| cardiomyocytes | | | | | | | | |
| Cytokine- | 2/294 | 0.700590403 | 0.751853115 | 0 | 0 | 0.821203462 | 0.292210361 | LEP; ACVR1B |
| cytokine | | | | | | | | |
| receptor | | | | | | | | |
| interaction | | | | | | | | |
| Retrograde | 1/148 | 0.707885149 | 0.755077492 | 0 | 0 | 0.816658371 | 0.282133758 | GNAI1 |
| endocannabinoid | | | | | | | | |
| signaling | | | | | | | | |
| Hepatitis C | 1/155 | 0.72446496 | 0.768107427 | 0 | 0 | 0.779260374 | 0.251172671 | PIK3CA |
| Hepatitis B | 1/163 | 0.742270706 | 0.777462227 | 0 | 0 | 0.740477266 | 0.220692784 | PIK3CA |
| Protein | 1/165 | 0.746540434 | 0.777462227 | 0 | 0 | 0.731372695 | 0.21378426 | SEL1L |
| processing in | | | | | | | | |
| endoplasmic | | | | | | | | |
| reticulum | | | | | | | | |
| RNA transport | 1/165 | 0.746540434 | 0.777462227 | 0 | 0 | 0.731372695 | 0.21378426 | PAIP1 |
| Influenza A | 1/171 | 0.758932317 | 0.785718164 | 0 | 0 | 0.705344333 | 0.19456407 | PIK3CA |
| Alcoholism | 1/180 | 0.776399864 | 0.799101614 | 0 | 0 | 0.669573511 | 0.169460755 | GNAI1 |
| Kaposi sarcoma- | 1/186 | 0.787339982 | 0.804581585 | 0 | 0 | 0.647659855 | 0.154852315 | PIK3CA |
| associated | | | | | | | | |
| herpesvirus | | | | | | | | |
| infection | | | | | | | | |
| Calcium | 1/188 | 0.790867126 | 0.804581585 | 0 | 0 | 0.640667797 | 0.150316879 | PTGER3 |
| signaling | | | | | | | | |
| pathway | | | | | | | | |
| Epstein-Barr | 1/201 | 0.812417916 | 0.821756053 | 0 | 0 | 0.598628049 | 0.124359228 | PIK3CA |
| virus infection | | | | | | | | |
| Thermogenesis | 1/231 | 0.854092328 | 0.858972855 | 0 | 0 | 0.519750795 | 0.081973005 | PLIN1 |
| Herpes simplex | 1/492 | 0.983865307 | 0.983865307 | 0 | 0 | 0.240226516 | 0.00390759 | PIK3CA |
| virus 1 infection | | | | | | | | |

Term, which pathway; Overlap, number of genes that overlap and total genes; P-value, enrichment p-value; Adjusted P-value, Q-value; Odds Ratio, enrichment; Combined Score, approximation of overall association (−log10(P) * log(Odds)), Genes, genes in the pathway which are co-expressed with COBLL1.

TABLE 12
List of WikiPathway pathways enriched among PAC-coexpressed genes.

| | | | | | Old | | | |
|---|---|---|---|---|---|---|---|---|
| | Over- | | Adjusted | Old | Adjusted | Odds | Combined | |
| Term | lap | P-value | P-value | P-value | P-value | Ratio | Score | Genes |
| Regulation of Actin | 8/150 | 3.53E−05 | 0.005716497 | 0 | 0 | 7.066654705 | 72.44731104 | PIK3CA; ROCK2; ITGA1; |
| Cytoskeleton WP51 | | | | | | | | FGF13; ARHGEF7; CRK; |
| | | | | | | | | FGFR2; ARHGEF6 |
| Integrin-mediated | 6/101 | 1.91E−04 | 0.015477604 | 0 | 0 | 7.841112214 | 67.14195699 | ITGAM; ROCK2; ITGA1; |
| Cell Adhesion WP185 | | | | | | | | ARHGEF7; CRK; TNS1 |
| Hippo-Merlin | 6/120 | 4.84E−04 | 0.026155061 | 0 | 0 | 6.527970871 | 49.82602113 | PPP1R14A; ITGAM; LATS2; |
| Signaling | | | | | | | | ITGA1; TEAD1; FGFR2 |
| Dysregulation | | | | | | | | |
| WP4541 | | | | | | | | |
| Transcription factor | 3/22 | 7.57E−04 | 0.026526441 | 0 | 0 | 19.31384016 | 138.8017185 | LEP; NRIP1; IRS2 |
| regulation in | | | | | | | | |
| adipogenesis | | | | | | | | |
| WP3599 | | | | | | | | |
| Hippo-Yap signaling | 3/23 | 8.65E−04 | 0.026526441 | 0 | 0 | 18.34722222 | 129.4017394 | LATS2; TEAD1; MAP4K3 |
| pathway WP4537 | | | | | | | | |
| Angiogenesis | 3/24 | 9.82E−04 | 0.026526441 | 0 | 0 | 17.47266314 | 121.0060571 | ANGPT1; PIK3CA; FGFR2 |
| WP1539 | | | | | | | | |
| RAC1/PAK1/p38/MM | 4/68 | 0.002413217 | 0.049221831 | 0 | 0 | 7.67507764 | 46.25611625 | PIK3CA; ANGPT1; TNIP1; |
| P2 Pathway WP3303 | | | | | | | | CRK |
| Endothelin Pathways | 3/33 | 0.00250833 | 0.049221831 | 0 | 0 | 12.22530864 | 73.20683819 | NPY1R; ECE1; GNAI1 |
| WP2197 | | | | | | | | |
| Signaling of | 3/34 | 0.002734546 | 0.049221831 | 0 | 0 | 11.83034648 | 69.82021816 | PIK3CA; ITGA1; CRK |
| Hepatocyte Growth | | | | | | | | |
| Factor Receptor | | | | | | | | |
| WP313 | | | | | | | | |
| Microglia Pathogen | 3/40 | 0.004354844 | 0.066253959 | 0 | 0 | 9.908908909 | 53.86945081 | ITGAM; FCER1G; PIK3CA |
| Phagocytosis | | | | | | | | |
| Pathway WP3937 | | | | | | | | |
| Adipogenesis WP236 | 5/130 | 0.004498726 | 0.066253959 | 0 | 0 | 4.9275 | 26.6280184 | MBNL1; LEP; NRIP1; |
| | | | | | | | | IRS2; PLIN1 |
| MicroRNAs in | 4/84 | 0.005165306 | 0.069731626 | 0 | 0 | 6.135093168 | 32.30611842 | PIK3CA; ROCK2; LRP5; |
| cardiomyocyte | | | | | | | | FGFR2 |
| hypertrophy WP1544 | | | | | | | | |
| Phytochemical | 2/15 | 0.006619586 | 0.08249023 | 0 | 0 | 18.70882492 | 93.8756898 | GCLC; PIK3CA |
| activity on NRF2 | | | | | | | | |
| transcriptional | | | | | | | | |
| activation WP3 | | | | | | | | |
| Leptin Insulin Overlap | 2/17 | 0.008481838 | 0.095589537 | 0 | 0 | 16.21267894 | 77.3316922 | LEP; IRS2 |
| WP3935 | | | | | | | | |
| Pathways Regulating | 4/98 | 0.008850883 | 0.095589537 | 0 | 0 | 5.21765561 | 24.66510012 | TCF7L2; LATS2; TEAD1; |
| Hippo Signaling | | | | | | | | FGFR2 |
| WP4540 | | | | | | | | |
| Transcription co- | 2/18 | 0.009490728 | 0.096093624 | 0 | 0 | 15.19861963 | 70.78665791 | LATS2; TEAD1 |
| factors SKI and SKIL | | | | | | | | |
| protein partners | | | | | | | | |
| WP4533 | | | | | | | | |
| Focal Adhesion-PI3K- | 7/303 | 0.013059057 | 0.123743442 | 0 | 0 | 2.924499658 | 12.68727902 | PIK3CA; ANGPT1; |
| Akt-mTOR-signaling | | | | | | | | CAB39L; IRS2; FGF13; |
| pathway WP3932 | | | | | | | | FGFR2; THBS4 |
| Wnt Signaling WP428 | 4/115 | 0.015219815 | 0.123743442 | 0 | 0 | 4.414750154 | 18.47642275 | TCF7L2; ROCK2; |
| | | | | | | | | CTNNBIP1; LRP5 |
| Endometrial cancer | 3/63 | 0.015253684 | 0.123743442 | 0 | 0 | 6.103395062 | 25.53010014 | TCF7L2; PIK3CA; FGFR2 |
| WP4155 | | | | | | | | |
| NRF2-ARE regulation | 2/23 | 0.015276968 | 0.123743442 | 0 | 0 | 11.57697926 | 48.40808453 | GCLC; PIK3CA |
| WP4357 | | | | | | | | |
| GPCRs, Class B | 2/24 | 0.016576472 | 0.127875645 | 0 | 0 | 11.0501952 | 45.30326885 | VIPR1; SCTR |
| Secretin-like WP334 | | | | | | | | |
| Vitamin D Receptor | 5/182 | 0.017632649 | 0.129840419 | 0 | 0 | 3.47069209 | 14.01466512 | LPGAT1; ITGAM; NRIP1; |
| Pathway WP2877 | | | | | | | | ID4; LRP5 |
| Arrhythmogenic Right | 3/74 | 0.023325028 | 0.157712253 | 0 | 0 | 5.154929577 | 19.37340243 | TCF7L2; ITGA1; DSG2 |
| Ventricular | | | | | | | | |
| Cardiomyopathy | | | | | | | | |
| WP2118 | | | | | | | | |
| Angiopoietin Like | 4/132 | 0.023912827 | 0.157712253 | 0 | 0 | 3.82511646 | 14.28046139 | PIK3CA; IRS2; CRK; |
| Protein 8 Regulatory | | | | | | | | MAP4K3 |
| Pathway WP3915 | | | | | | | | |
| Focal Adhesion | 5/198 | 0.024338311 | 0.157712253 | 0 | 0 | 3.180375648 | 11.81733325 | PIK3CA; ROCK2; ITGA1; |
| WP306 | | | | | | | | CRK; THBS4 |
| Ovarian Infertility | 2/32 | 0.028539894 | 0.177825493 | 0 | 0 | 8.100204499 | 28.8079916 | GJA4; NRIP1 |
| Genes WP34 | | | | | | | | |
| ncRNAs involved in | 3/86 | 0.03430246 | 0.202346169 | 0 | 0 | 4.406961178 | 14.86264501 | TCF7L2; CTNNBIP1; LRP5 |
| Wnt signaling in | | | | | | | | |
| hepatocellular | | | | | | | | |
| carcinoma WP4336 | | | | | | | | |
| Catalytic cycle of | 1/5 | 0.040578743 | 0.202346169 | 0 | 0 | 30.23018293 | 96.87295099 | FM03 |
| mammalian Flavin- | | | | | | | | |
| containing | | | | | | | | |
| MonoOxygenases | | | | | | | | |
| (FMOs) WP688 | | | | | | | | |
| Sulindac Metabolic | 1/5 | 0.040578743 | 0.202346169 | 0 | 0 | 30.23018293 | 96.87295099 | MSRB3 |
| Pathway WP2542 | | | | | | | | |
| G13 Signaling | 2/39 | 0.041075821 | 0.202346169 | 0 | 0 | 6.565412038 | 20.95899875 | PIK3CA; ROCK2 |
| Pathway WP524 | | | | | | | | |
| Corticotropin- | 3/93 | 0.041739203 | 0.202346169 | 0 | 0 | 4.062757202 | 12.90459446 | CRHBP; ECE1; GNAI1 |
| releasing hormone | | | | | | | | |
| signaling pathway | | | | | | | | |
| WP2355 | | | | | | | | |
| Sudden Infant Death | 4/158 | 0.042069876 | 0.202346169 | 0 | 0 | 3.175123014 | 10.06013381 | VIPR1; ECE1; FMO3; |
| Syndrome (SIDS) | | | | | | | | SPTBN1 |
| Susceptibility | | | | | | | | |
| Pathways WP706 | | | | | | | | |
| LncRNA involvement | 3/94 | 0.042862594 | 0.202346169 | 0 | 0 | 4.017908018 | 12.655429 | TCF7L2; CTNNBIP1; LRP5 |
| in canonical Wnt | | | | | | | | |
| signaling and | | | | | | | | |
| colorectal cancer | | | | | | | | |
| WP4258 | | | | | | | | |
| Epithelial to | 4/159 | 0.042888833 | 0.202346169 | 0 | 0 | 3.154478061 | 9.933905023 | TJP1; LATS2; PIK3CA; |
| mesenchymal | | | | | | | | LRP5 |
| transition in | | | | | | | | |
| colorectal cancer | | | | | | | | |
| WP4239 | | | | | | | | |
| Insulin Signaling | 4/160 | 0.043716765 | 0.202346169 | 0 | 0 | 3.134097786 | 9.809800074 | PIK3CA; IRS2; CRK; |
| WP481 | | | | | | | | MAP4K3 |
| Chemokine signaling | 4/164 | 0.047118189 | 0.212031851 | 0 | 0 | 3.055124224 | 9.333698314 | PIK3CA; ROCK2; CRK; |
| pathway WP3929 | | | | | | | | GNAI1 |
| Nicotine Metabolism | 1/6 | 0.048495928 | 0.212333521 | 0 | 0 | 24.18292683 | 73.18419779 | FMO3 |
| WP1600 | | | | | | | | |
| Wnt Signaling | 3/102 | 0.052387142 | 0.222092865 | 0 | 0 | 3.691732136 | 10.88726544 | TCF7L2; LRP5; FBXW2 |
| Pathway and | | | | | | | | |
| Pluripotency WP399 | | | | | | | | |
| MAPK Signaling | 5/246 | 0.053466801 | 0.222092865 | 0 | 0 | 2.540715768 | 7.440979948 | PPM1A; FGF13; CRK; |
| Pathway WP382 | | | | | | | | FGFR2; MAP4K3 |
| Aryl Hydrocarbon | 2/46 | 0.055282054 | 0.223892321 | 0 | 0 | 5.518962632 | 15.97909079 | GCLC; NRIP1 |
| Receptor WP2586 | | | | | | | | |
| Mechanoregulation | 2/47 | 0.057434454 | 0.226936137 | 0 | 0 | 5.396046353 | 15.41710287 | TEAD1; MAP4K3 |
| and pathology of | | | | | | | | |
| YAP/TAZ via Hippo | | | | | | | | |
| and non-Hippo | | | | | | | | |
| mechanisms WP4534 | | | | | | | | |
| PI3K-Akt Signaling | 6/340 | 0.063106975 | 0.243412617 | 0 | 0 | 2.20325387 | 6.087422954 | PIK3CA; ANGPT1; ITGA1; |
| Pathway WP4172 | | | | | | | | FGF13; FGFR2; THBS4 |
| Copper homeostasis | 2/52 | 0.068616652 | 0.245253102 | 0 | 0 | 4.855214724 | 13.00818855 | PIK3CA; SLC31A2 |
| WP3286 | | | | | | | | |
| Wnt Signaling | 2/52 | 0.068616652 | 0.245253102 | 0 | 0 | 4.855214724 | 13.00818855 | TCF7L2; LRP5 |
| Pathway WP363 | | | | | | | | |
| ESC Pluripotency | 3/116 | 0.071265752 | 0.245253102 | 0 | 0 | 3.232055064 | 8.536954385 | LRP5; FGF13; FGFR2 |
| Pathways WP3931 | | | | | | | | |
| Thiamine metabolic | 1/9 | 0.071859962 | 0.245253102 | 0 | 0 | 15.11204268 | 39.79055287 | SLC19A3 |
| pathways WP4297 | | | | | | | | |
| IL-4 Signaling | 2/54 | 0.073274469 | 0.245253102 | 0 | 0 | 4.668003775 | 12.20002878 | PIK3CA; IRS2 |
| Pathway WP395 | | | | | | | | |
| Interferon type I | 2/54 | 0.073274469 | 0.245253102 | 0 | 0 | 4.668003775 | 12.20002878 | IRS2; CRK |
| signaling pathways | | | | | | | | |
| WP585 | | | | | | | | |
| Spinal Cord Injury | 3/118 | 0.074181494 | 0.245253102 | 0 | 0 | 3.175523349 | 8.260300163 | ROCK2; LEP; AIF1 |
| WP2431 | | | | | | | | |
| Gamma-Glutamyl | 1/10 | 0.07952055 | 0.252594687 | 0 | 0 | 13.43224932 | 34.00696028 | GCLC |
| Cycle for the | | | | | | | | |
| biosynthesis and | | | | | | | | |
| degradation of | | | | | | | | |
| glutathione, including | | | | | | | | |
| diseases WP4518 | | | | | | | | |
| Leptin and | 1/10 | 0.07952055 | 0.252594687 | 0 | 0 | 13.43224932 | 34.00696028 | LEP |
| adiponectin WP3934 | | | | | | | | |
| Circadian rhythm | 4/201 | 0.085252119 | 0.260854522 | 0 | 0 | 2.476652899 | 6.097871888 | ROCK2; LEP; NRIP1; |
| related genes | | | | | | | | ID4 |
| WP3594 | | | | | | | | |
| MET in type 1 | 2/59 | 0.085341294 | 0.260854522 | 0 | 0 | 4.25745345 | 10.47800522 | PIK3CA; CRK |
| papillary renal cell | | | | | | | | |
| carcinoma WP4205 | | | | | | | | |
| TYROBP Causal | 2/61 | 0.090325471 | 0.270976412 | 0 | 0 | 4.112717064 | 9.888352838 | ITGAM; CD37 |
| Network WP3945 | | | | | | | | |
| T-Cell antigen | 2/62 | 0.092849186 | 0.273483056 | 0 | 0 | 4.04396728 | 9.61161554 | DLG1; PIK3CA |
| Receptor (TCR) | | | | | | | | |
| pathway during | | | | | | | | |
| Staphylococcus | | | | | | | | |
| aureus infection | | | | | | | | |
| WP3863 | | | | | | | | |
| TGF-beta Signaling | 3/132 | 0.096014425 | 0.277756014 | 0 | 0 | 2.828883147 | 6.628799791 | PPM1A; ZFYVE16; |
| Pathway WP366 | | | | | | | | SPTBN1 |
| Cell-type Dependent | 1/13 | 0.102127274 | 0.278486281 | 0 | 0 | 10.0726626 | 22.98113689 | GNAI1 |
| Selectivity of CCK2R | | | | | | | | |
| Signaling WP3679 | | | | | | | | |
| Genes targeted by | 1/13 | 0.102127274 | 0.278486281 | 0 | 0 | 10.0726626 | 22.98113689 | PTBP2 |
| miRNAs in adipocytes | | | | | | | | |
| WP1992 | | | | | | | | |
| Mammary gland | 1/13 | 0.102127274 | 0.278486281 | 0 | 0 | 10.0726626 | 22.98113689 | NRIP1 |
| development | | | | | | | | |
| pathway - Puberty | | | | | | | | |
| (Stage 2 of 4) | | | | | | | | |
| WP2814 | | | | | | | | |
| Human Thyroid | 2/66 | 0.103143067 | 0.278486281 | 0 | 0 | 3.790452454 | 8.610536789 | PIK3CA; GNAI1 |
| Stimulating Hormone | | | | | | | | |
| (TSH) signaling | | | | | | | | |
| pathway WP2032 | | | | | | | | |
| Cysteine and | 1/14 | 0.10953953 | 0.290185727 | 0 | 0 | 9.297373358 | 20.56086033 | GCLC |
| methionine | | | | | | | | |
| catabolismwP4504 | | | | | | | | |
| AMP-activated | 2/69 | 0.111058735 | 0.290185727 | 0 | 0 | 3.620181302 | 7.956058228 | PIK3CA; LEP |
| Protein Kinase | | | | | | | | |
| (AMPK) Signaling | | | | | | | | |
| WP1403 | | | | | | | | |
| Brain-Derived | 3/144 | 0.116554827 | 0.299712413 | 0 | 0 | 2.58655109 | 5.559516088 | DLG1; RANBP9; IRS2 |
| Neurotrophic Factor | | | | | | | | |
| (BDNF) signaling | | | | | | | | |
| pathway WP2380 | | | | | | | | |
| Primary Focal | 2/72 | 0.119127616 | 0.301541777 | 0 | 0 | 3.46450482 | 7.370941734 | LRP5; UTRN |
| Segmental | | | | | | | | |
| Glomerulosclerosis | | | | | | | | |
| FSGS WP2572 | | | | | | | | |
| Nuclear Receptors | 5/319 | 0.124689727 | 0.304128018 | 0 | 0 | 1.942774682 | 4.044714698 | EPB41L4B; GCLC; NRIP1; |
| Meta-Pathway | | | | | | | | IRS2; FGF13 |
| WP2882 | | | | | | | | |
| Prolactin Signaling | 2/76 | 0.130103483 | 0.304128018 | 0 | 0 | 3.27657105 | 6.682321303 | PIK3CA; IRS2 |
| Pathway WP2037 | | | | | | | | |
| Leptin signaling | 2/76 | 0.130103483 | 0.304128018 | 0 | 0 | 3.27657105 | 6.682321303 | ROCK2; LEP |
| pathway WP2034 | | | | | | | | |
| NOTCH1 regulation of | 1/17 | 0.131413341 | 0.304128018 | 0 | 0 | 7.552972561 | 15.32806027 | ITGA1 |
| human endothelial | | | | | | | | |
| cell calcification | | | | | | | | |
| WP3413 | | | | | | | | |
| Amplification and | 1/17 | 0.131413341 | 0.304128018 | 0 | 0 | 7.552972561 | 15.32806027 | TCF7L2 |
| Expansion of | | | | | | | | |
| Oncogenic Pathways | | | | | | | | |
| as Metastatic Traits | | | | | | | | |
| WP3678 | | | | | | | | |
| miR-509-3p | 1/17 | 0.131413341 | 0.304128018 | 0 | 0 | 7.552972561 | 15.32806027 | TEAD1 |
| alteration of | | | | | | | | |
| YAP1/ECM axis | | | | | | | | |
| WP3967 | | | | | | | | |
| Breast cancer | 3/154 | 0.134811963 | 0.307552113 | 0 | 0 | 2.414029924 | 4.837412608 | TCF7L2; PIK3CA; LRP5 |
| pathway WP4262 | | | | | | | | |
| Nonalcoholic fatty | 3/155 | 0.136689828 | 0.307552113 | 0 | 0 | 2.398026316 | 4.772170566 | PIK3CA; LEP; IRS2 |
| liver disease WP4396 | | | | | | | | |
| Small Ligand GPCRs | 1/19 | 0.145698329 | 0.317472059 | 0 | 0 | 6.713075881 | 12.93084111 | PTGER3 |
| WP247 | | | | | | | | |
| Farnesoid X Receptor | 1/19 | 0.145698329 | 0.317472059 | 0 | 0 | 6.713075881 | 12.93084111 | IRS2 |
| Pathway WP2879 | | | | | | | | |
| Signaling Pathways in | 2/82 | 0.146977805 | 0.317472059 | 0 | 0 | 3.029907975 | 5.80976882 | PIK3CA; FGFR2 |
| Glioblastoma | | | | | | | | |
| WP2261 | | | | | | | | |
| Viral Acute | 2/84 | 0.152699152 | 0.321376462 | 0 | 0 | 2.955708514 | 5.554620507 | BNIP2; AIF1 |
| Myocarditis WP4298 | | | | | | | | |
| BMP Signaling | 1/20 | 0.15275301 | 0.321376462 | 0 | 0 | 6.359435173 | 11.94895247 | FGFR2 |
| Pathway in Eyelid | | | | | | | | |
| Development | | | | | | | | |
| WP3927 | | | | | | | | |
| Nicotine Activity on | 1/21 | 0.159749784 | 0.322428216 | 0 | 0 | 6.041158537 | 11.08037003 | GNAI1 |
| Dopaminergic | | | | | | | | |
| Neurons WP1602 | | | | | | | | |
| Tamoxifen | 1/21 | 0.159749784 | 0.322428216 | 0 | 0 | 6.041158537 | 11.08037003 | FM03 |
| metabolism WP691 | | | | | | | | |
| PI3K/AKT/mTOR - | 1/22 | 0.166689124 | 0.322428216 | 0 | 0 | 5.753193961 | 10.30756459 | PIK3CA |
| VitD3 Signalling | | | | | | | | |
| WP4141 | | | | | | | | |
| Regulation of | 1/22 | 0.166689124 | 0.322428216 | 0 | 0 | 5.753193961 | 10.30756459 | BCL2L13 |
| Apoptosis by | | | | | | | | |
| Parathyroid | | | | | | | | |
| Hormone-related | | | | | | | | |
| Protein WP3872 | | | | | | | | |
| miRNA targets in | 1/22 | 0.166689124 | 0.322428216 | 0 | 0 | 5.753193961 | 10.30756459 | ITGA1 |
| ECM and membrane | | | | | | | | |
| receptors WP2911 | | | | | | | | |
| Methionine De Novo | 1/22 | 0.166689124 | 0.322428216 | 0 | 0 | 5.753193961 | 10.30756459 | MSRB3 |
| and Salvage Pathway | | | | | | | | |
| WP3580 | | | | | | | | |
| Pancreatic | 2/89 | 0.167185001 | 0.322428216 | 0 | 0 | 2.78513504 | 4.981643733 | PIK3CA; ARHGEF6 |
| adenocarcinoma | | | | | | | | |
| pathway WP4263 | | | | | | | | |
| ErbB Signaling | 2/91 | 0.173044787 | 0.323202103 | 0 | 0 | 2.722272007 | 4.775422718 | PIK3CA; CRK |
| Pathway WP673 | | | | | | | | |
| Estrogen signaling | 1/23 | 0.1735715 | 0.323202103 | 0 | 0 | 5.491407982 | 9.616365099 | PIK3CA |
| pathway WP712 | | | | | | | | |
| Glutathione | 1/23 | 0.1735715 | 0.323202103 | 0 | 0 | 5.491407982 | 9.616365099 | GCLC |
| metabolism WP100 | | | | | | | | |
| Photodynamic | 1/24 | 0.180397375 | 0.324715274 | 0 | 0 | 5.252386002 | 8.995200678 | GCLC |
| therapy-induced | | | | | | | | |
| NFE2L2 (NRF2) | | | | | | | | |
| survival signaling | | | | | | | | |
| WP3612 | | | | | | | | |
| IL1 and | 1/24 | 0.180397375 | 0.324715274 | 0 | 0 | 5.252386002 | 8.995200678 | PIK3CA |
| megakaryocytes in | | | | | | | | |
| obesity WP2865 | | | | | | | | |
| miRNA regulation of | 1/24 | 0.180397375 | 0.324715274 | 0 | 0 | 5.252386002 | 8.995200678 | SIAH1 |
| p53 pathway in | | | | | | | | |
| prostate cancer | | | | | | | | |
| WP3982 | | | | | | | | |
| Differentiation of | 1/25 | 0.18716721 | 0.327174971 | 0 | 0 | 5.03328252 | 8.43453772 | LEP |
| white and brown | | | | | | | | |
| adipocyte WP2895 | | | | | | | | |
| Signal Transduction | 1/25 | 0.18716721 | 0.327174971 | 0 | 0 | 5.03328252 | 8.43453772 | GNAI1 |
| of S1P Receptor WP26 | | | | | | | | |
| Metapathway | 3/183 | 0.192507859 | 0.327174971 | 0 | 0 | 2.022119342 | 3.331680833 | GLYAT; FMO3; CHST3 |
| biotransformation | | | | | | | | |
| Phase I and II WP702 | | | | | | | | |
| EPO Receptor | 1/26 | 0.193881464 | 0.327174971 | 0 | 0 | 4.831707317 | 7.926456035 | IRS2 |
| Signaling WP581 | | | | | | | | |
| Glucuronidation | 1/26 | 0.193881464 | 0.327174971 | 0 | 0 | 4.831707317 | 7.926456035 | UGP2 |
| WP698 | | | | | | | | |
| Wnt/beta-catenin | 1/26 | 0.193881464 | 0.327174971 | 0 | 0 | 4.831707317 | 7.926456035 | LRP5 |
| Signaling Pathway in | | | | | | | | |
| Leukemia WP3658 | | | | | | | | |
| Sphingolipid pathway | 1/27 | 0.200540589 | 0.331505872 | 0 | 0 | 4.645637899 | 7.464325789 | SERINC1 |
| WP1422 | | | | | | | | |
| Intraflagellar | 1/27 | 0.200540589 | 0.331505872 | 0 | 0 | 4.645637899 | 7.464325789 | DYNC1LI2 |
| transport proteins | | | | | | | | |
| binding to dynein | | | | | | | | |
| WP4532 | | | | | | | | |
| Nanoparticle- | 1/28 | 0.207145036 | 0.338964604 | 0 | 0 | 4.4733514 | 7.04255848 | ITGA1 |
| mediated activation | | | | | | | | |
| of receptor signaling | | | | | | | | |
| WP2643 | | | | | | | | |
| PDGFR-beta pathway | 1/29 | 0.21369525 | 0.342758718 | 0 | 0 | 4.31337108 | 6.656412982 | PIK3CA |
| WP3972 | | | | | | | | |
| Lipid Metabolism | 1/29 | 0.21369525 | 0.342758718 | 0 | 0 | 4.31337108 | 6.656412982 | PLIN1 |
| Pathway WP3965 | | | | | | | | |
| PI3K-AKT-mTOR | 1/30 | 0.220191675 | 0.34971619 | 0 | 0 | 4.164423886 | 6.30184302 | PIK3CA |
| signaling pathway | | | | | | | | |
| and therapeutic | | | | | | | | |
| opportunities WP3844 | | | | | | | | |
| Trans-sulfuration and | 1/31 | 0.226634749 | 0.355749895 | 0 | 0 | 4.025406504 | 5.975376181 | GCLC |
| one carbon | | | | | | | | |
| metabolism WP2525 | | | | | | | | |
| DNA Damage | 2/110 | 0.230016298 | 0.355749895 | 0 | 0 | 2.241195183 | 3.293671897 | TCF7L2; PIK3CA |
| Response (only ATM | | | | | | | | |
| dependent) WP710 | | | | | | | | |
| Alpha 6 Beta 4 | 1/33 | 0.239362583 | 0.355749895 | 0 | 0 | 3.7734375 | 5.395169587 | IRS2 |
| signaling pathway | | | | | | | | |
| WP244 | | | | | | | | |
| Oxidative Stress | 1/33 | 0.239362583 | 0.355749895 | 0 | 0 | 3.7734375 | 5.395169587 | GCLC |
| WP408 | | | | | | | | |
| Pregnane X Receptor | 1/33 | 0.239362583 | 0.355749895 | 0 | 0 | 3.7734375 | 5.395169587 | NRIP1 |
| pathway WP2876 | | | | | | | | |
| Resistin as a | 1/33 | 0.239362583 | 0.355749895 | 0 | 0 | 3.7734375 | 5.395169587 | PIK3CA |
| regulator of | | | | | | | | |
| inflammation | | | | | | | | |
| WP4481 | | | | | | | | |
| miRNA regulation of | 1/33 | 0.239362583 | 0.355749895 | 0 | 0 | 3.7734375 | 5.395169587 | PIK3CA |
| prostate cancer | | | | | | | | |
| signaling pathways | | | | | | | | |
| WP3981 | | | | | | | | |
| Wnt Signaling in | 1/36 | 0.258064978 | 0.378760258 | 0 | 0 | 3.449477352 | 4.672468415 | LRP5 |
| Kidney Disease | | | | | | | | |
| WP4150 | | | | | | | | |
| Photodynamic | 1/37 | 0.26419697 | 0.378760258 | 0 | 0 | 3.35348916 | 4.463696472 | ANGPT1 |
| therapy-induced HIF- | | | | | | | | |
| 1 survival signaling | | | | | | | | |
| WP3614 | | | | | | | | |
| Fibrin Complement | 1/37 | 0.26419697 | 0.378760258 | 0 | 0 | 3.35348916 | 4.463696472 | ITGAM |
| Receptor 3 Signaling | | | | | | | | |
| Pathway WP4136 | | | | | | | | |
| Melatonin | 1/37 | 0.26419697 | 0.378760258 | 0 | 0 | 3.35348916 | 4.463696472 | ECE1 |
| metabolism and | | | | | | | | |
| effects WP3298 | | | | | | | | |
| Bladder Cancer | 1/40 | 0.282292336 | 0.394235849 | 0 | 0 | 3.095059412 | 3.914668568 | DAPK2 |
| WP2828 | | | | | | | | |
| Ferroptosis WP4313 | 1/40 | 0.282292336 | 0.394235849 | 0 | 0 | 3.095059412 | 3.914668568 | GCLC |
| Glycogen Synthesis | 1/40 | 0.282292336 | 0.394235849 | 0 | 0 | 3.095059412 | 3.914668568 | UGP2 |
| and Degradation | | | | | | | | |
| WP500 | | | | | | | | |
| Ebola Virus Pathway | 2/129 | 0.288022591 | 0.398800511 | 0 | 0 | 1.904062606 | 2.370017875 | PIK3CA; ITGA1 |
| on Host WP4217 | | | | | | | | |
| Common Pathways | 1/42 | 0.294109476 | 0.403777417 | 0 | 0 | 2.943783462 | 3.602611658 | GNAI1 |
| Underlying Drug | | | | | | | | |
| Addiction WP2636 | | | | | | | | |
| Vitamin A and | 1/43 | 0.299945319 | 0.408328922 | 0 | 0 | 2.8735482 | 3.46019769 | RDH10 |
| Carotenoid | | | | | | | | |
| Metabolism WP716 | | | | | | | | |
| VEGFA-VEGFR2 | 3/236 | 0.308817266 | 0.416903309 | 0 | 0 | 1.557939914 | 1.830588044 | PIK3CA; ROCK2; CRK |
| Signaling Pathway | | | | | | | | |
| WP3888 | | | | | | | | |
| Prostaglandin | 1/45 | 0.311473526 | 0.417014142 | 0 | 0 | 2.742655211 | 3.199145303 | PTGER3 |
| Synthesis and | | | | | | | | |
| Regulation WP98 | | | | | | | | |
| Regulation of | 1/46 | 0.31716667 | 0.419411867 | 0 | 0 | 2.681571816 | 3.079323653 | PIK3CA |
| Microtubule | | | | | | | | |
| Cytoskeleton | | | | | | | | |
| WP2038 | | | | | | | | |
| Regulation of toll-like | 2/139 | 0.318442344 | 0.419411867 | 0 | 0 | 1.764184318 | 2.018780539 | PIK3CA; RNF41 |
| receptor signaling | | | | | | | | |
| pathway WP1449 | | | | | | | | |
| Thymic Stromal | 1/47 | 0.322813023 | 0.421739594 | 0 | 0 | 2.623144221 | 2.965941956 | PIK3CA |
| LymphoPoietin (TSLP) | | | | | | | | |
| Signaling Pathway | | | | | | | | |
| WP2203 | | | | | | | | |
| Rett syndrome | 1/48 | 0.328412966 | 0.422245242 | 0 | 0 | 2.567202906 | 2.858537872 | CRK |
| causing genes | | | | | | | | |
| WP4312 | | | | | | | | |
| Exercise-induced | 1/48 | 0.328412966 | 0.422245242 | 0 | 0 | 2.567202906 | 2.858537872 | UGP2 |
| Circadian Regulation | | | | | | | | |
| WP410 | | | | | | | | |
| Synaptic signaling | 1/50 | 0.339475139 | 0.429758889 | 0 | 0 | 2.462170234 | 2.66001685 | PIK3CA |
| pathways associated | | | | | | | | |
| with autism spectrum | | | | | | | | |
| disorder WP4539 | | | | | | | | |
| NRF2 pathway | 2/146 | 0.339562579 | 0.429758889 | 0 | 0 | 1.677828903 | 1.812218003 | GCLC; FGF13 |
| WP2884 | | | | | | | | |
| Calcium Regulation in | 2/149 | 0.348555201 | 0.4332649 | 0 | 0 | 1.643337089 | 1.732009364 | GJA4; GNAI1 |
| the Cardiac Cell | | | | | | | | |
| WP536 | | | | | | | | |
| One carbon | 1/52 | 0.350356185 | 0.4332649 | 0 | 0 | 2.365375418 | 2.480817499 | GCLC |
| metabolism and | | | | | | | | |
| related pathways | | | | | | | | |
| WP3940 | | | | | | | | |
| Translation Factors | 1/52 | 0.350356185 | 0.4332649 | 0 | 0 | 2.365375418 | 2.480817499 | PAIP1 |
| WP107 | | | | | | | | |
| Apoptosis-related | 1/53 | 0.355729708 | 0.436577369 | 0 | 0 | 2.319770169 | 2.397677523 | GCLC |
| network due to | | | | | | | | |
| altered Notch3 in | | | | | | | | |
| ovarian cancer | | | | | | | | |
| WP2864 | | | | | | | | |
| Cardiac Hypertrophic | 1/55 | 0.366344576 | 0.439613492 | 0 | 0 | 2.23362692 | 2.242965542 | FGFR2 |
| Response WP2795 | | | | | | | | |
| Pathogenic | 1/55 | 0.366344576 | 0.439613492 | 0 | 0 | 2.23362692 | 2.242965542 | ROCK2 |
| Escherichia coli | | | | | | | | |
| infection WP2272 | | | | | | | | |
| Hematopoietic Stem | 1/55 | 0.366344576 | 0.439613492 | 0 | 0 | 2.23362692 | 2.242965542 | ACVR1B |
| Cell Differentiation | | | | | | | | |
| WP2849 | | | | | | | | |
| EGF/EGFR Signaling | 2/162 | 0.387010951 | 0.457686189 | 0 | 0 | 1.508819018 | 1.43232535 | PXDN; CRK |
| Pathway WP437 | | | | | | | | |
| Kit receptor signaling | 1/59 | 0.387055604 | 0.457686189 | 0 | 0 | 2.079163162 | 1.973514471 | CRK |
| pathway WP304 | | | | | | | | |
| Genotoxicity pathway | 1/63 | 0.407093659 | 0.477892557 | 0 | 0 | 1.944630212 | 1.747662505 | SEL1L |
| WP4286 | | | | | | | | |
| Pathways Affected in | 1/65 | 0.416867143 | 0.484499106 | 0 | 0 | 1.88366997 | 1.648188073 | PIK3CA |
| Adenoid Cystic | | | | | | | | |
| Carcinoma WP3651 | | | | | | | | |
| Non-small cell lung | 1/66 | 0.421693666 | 0.484499106 | 0 | 0 | 1.854596623 | 1.60139993 | PIK3CA |
| cancer WP4255 | | | | | | | | |
| Association Between | 1/66 | 0.421693666 | 0.484499106 | 0 | 0 | 1.854596623 | 1.60139993 | ROCK2 |
| Physico-Chemical | | | | | | | | |
| Features and Toxicity | | | | | | | | |
| Associated Pathways | | | | | | | | |
| WP3680 | | | | | | | | |
| PPAR signaling | 1/67 | 0.42648048 | 0.486548154 | 0 | 0 | 1.826404287 | 1.556441058 | PLIN1 |
| pathway WP3942 | | | | | | | | |
| Sterol Regulatory | 1/69 | 0.43593628 | 0.493857883 | 0 | 0 | 1.772507174 | 1.471640377 | PIK3CA |
| Element-Binding | | | | | | | | |
| Proteins (SREBP) | | | | | | | | |
| signalling WP1982 | | | | | | | | |
| Parkin-Ubiquitin | 1/70 | 0.440605906 | 0.495681645 | 0 | 0 | 1.746730293 | 1.431627903 | SIAH1 |
| Proteasomal System | | | | | | | | |
| pathway WP2359 | | | | | | | | |
| Ras Signaling WP4223 | 2/184 | 0.449680824 | 0.502402024 | 0 | 0 | 1.324951122 | 1.058923764 | PIK3CA; FGFR2 |
| Chromosomal and | 1/73 | 0.454385488 | 0.504181157 | 0 | 0 | 1.673695799 | 1.320226895 | TCF7L2 |
| microsatellite | | | | | | | | |
| instability in | | | | | | | | |
| colorectal cancer | | | | | | | | |
| WP4216 | | | | | | | | |
| Peptide GPCRs WP24 | 1/74 | 0.458903288 | 0.505730154 | 0 | 0 | 1.650684932 | 1.285744562 | NPY1R |
| Pathways in clear cell | 1/85 | 0.506210687 | 0.554095482 | 0 | 0 | 1.433725319 | 0.976083522 | PBRM1 |
| renal cell carcinoma | | | | | | | | |
| WP4018 | | | | | | | | |
| Androgen receptor | 1/90 | 0.526332208 | 0.568438785 | 0 | 0 | 1.352836394 | 0.868281094 | ROCK2 |
| signaling pathway | | | | | | | | |
| WP138 | | | | | | | | |
| T-Cell antigen | 1/90 | 0.526332208 | 0.568438785 | 0 | 0 | 1.352836394 | 0.868281094 | CRK |
| Receptor (TCR) | | | | | | | | |
| Signaling Pathway | | | | | | | | |
| WP69 | | | | | | | | |
| GPCRs, Other WP117 | 1/91 | 0.530257622 | 0.568885661 | 0 | 0 | 1.337737127 | 0.848650148 | ADGRF5 |
| TNF alpha Signaling | 1/92 | 0.5341507 | 0.569292194 | 0 | 0 | 1.322969713 | 0.829604236 | OTUD7B |
| Pathway WP231 | | | | | | | | |
| G Protein Signaling | 1/93 | 0.538011708 | 0.569659455 | 0 | 0 | 1.30852333 | 0.811120844 | GNAI1 |
| Pathways WP35 | | | | | | | | |
| B Cell Receptor | 1/97 | 0.553140249 | 0.581874807 | 0 | 0 | 1.253747459 | 0.742398653 | CRK |
| Signaling Pathway | | | | | | | | |
| WP23 | | | | | | | | |
| Neural Crest | 1/101 | 0.567776285 | 0.593417795 | 0 | 0 | 1.203353659 | 0.681131626 | FGFR2 |
| Differentiation | | | | | | | | |
| WP2064 | | | | | | | | |
| Toll-like Receptor | 1/103 | 0.574914615 | 0.597026715 | 0 | 0 | 1.179638929 | 0.652969954 | PIK3CA |
| Signaling Pathway | | | | | | | | |
| WP75 | | | | | | | | |
| Thermogenesis | 1/108 | 0.592251908 | 0.611113434 | 0 | 0 | 1.124230682 | 0.588898128 | PLIN1 |
| WP4321 | | | | | | | | |
| GPCRs, Class A | 2/257 | 0.628580726 | 0.644494162 | 0 | 0 | 0.942138819 | 0.437426402 | PTGER3; NPY1R |
| Rhodopsin-like | | | | | | | | |
| WP455 | | | | | | | | |
| mRNA Processing | 1/126 | 0.649045434 | 0.661291574 | 0 | 0 | 0.961463415 | 0.415595021 | PTBP2 |
| WP411 | | | | | | | | |
| Ectoderm | 1/138 | 0.682465702 | 0.690996523 | 0 | 0 | 0.876713548 | 0.33494228 | FGFR2 |
| Differentiation | | | | | | | | |
| WP2858 | | | | | | | | |
| Mesodermal | 1/147 | 0.705437022 | 0.709818619 | 0 | 0 | 0.822293685 | 0.286929332 | TEAD1 |
| Commitment | | | | | | | | |
| Pathway WP2857 | | | | | | | | |
| Ciliary landscape | 1/216 | 0.834554307 | 0.834554307 | 0 | 0 | 0.55643789 | 0.100635944 | RANBP9 |
| WP4352 | | | | | | | | |

Term, which pathway; Overlap, number of genes that overlap and total genes; P-value, enrichment p-value; Adjusted P-value, Q-value; Odds Ratio, enrichment; Combined Score, approximation of overall association (−log10(P) * log(Odds)), Genes, genes in the pathway which are co-expressed with COBLL1.

TABLE 13
List of HCI pathways enriched among PAC-coexpressed genes.

| | | | | | Old | | | |
|---|---|---|---|---|---|---|---|---|
| | | | Adjusted | Old P- | Adjusted | Odds | Combined | |
| Term | Overlap | P-value | P-value | value | P-value | Ratio | Score | Genes |
| Integrin-linked kinase | 5/45 | 3.37E−05 | 0.003600713 | 0 | 0 | 15.46484375 | 159.279421 | PPP1R14A; ARHGEF7; |
| signaling Homo sapiens | | | | | | | | LIMS2; TNS1; |
| 21738158-6194-11e5- | | | | | | | | ARHGEF6 |
| 8ac5-06603eb7f303 | | | | | | | | |
| Signaling events | 5/58 | 1.16E−04 | 0.006193572 | 0 | 0 | 11.66391509 | 105.7208509 | PIK3CA; ROCK2; |
| mediated by focal | | | | | | | | PTPN21; ARHGEF7; |
| adhesion kinase Homo | | | | | | | | CRK |
| sapiens 8fb80085-6195- | | | | | | | | |
| 11e5-8ac5-06603eb7f303 | | | | | | | | |
| E-cadherin signaling in | 4/39 | 2.93E−04 | 0.01046151 | 0 | 0 | 14.05501331 | 114.3272589 | TJP1; DLG1; PIK3CA; |
| the nascent adherens | | | | | | | | CRK |
| junction Homo sapiens | | | | | | | | |
| aef0d8c2-6191-11e5- | | | | | | | | |
| 8ac5-06603eb7f303 | | | | | | | | |
| VEGFR3 signaling in | 3/25 | 0.001109703 | 0.029052118 | 0 | 0 | 16.67760943 | 113.4688246 | PIK3CA; ITGA1; CRK |
| lymphatic endothelium | | | | | | | | |
| Homo sapiens 9048d98c- | | | | | | | | |
| 6196-11e5-8ac5- | | | | | | | | |
| 06603eb7f303 | | | | | | | | |
| IGF1 pathway Homo | 3/28 | 0.001552216 | 0.029052118 | 0 | 0 | 14.67407407 | 94.91296294 | PIK3CA; IRS2; CRK |
| sapiens 5e904cd6-6193- | | | | | | | | |
| 11e5-8ac5-06603eb7f303 | | | | | | | | |
| Nectin adhesion pathway | 3/29 | 0.001720898 | 0.029052118 | 0 | 0 | 14.10897436 | 89.80233628 | PIK3CA; PTPRM; CRK |
| Homo sapiens 685baa82- | | | | | | | | |
| 6194-11e5-8ac5- | | | | | | | | |
| 06603eb7f303 | | | | | | | | |
| Regulation of CDC42 | 3/30 | 0.001900606 | 0.029052118 | 0 | 0 | 13.58573388 | 85.12253726 | ITSN1; ARHGEF7; |
| activity Homo sapiens | | | | | | | | ARHGEF6 |
| Offe6681-6195-11e5- | | | | | | | | |
| 8ac5-06603eb7f303 | | | | | | | | |
| CDC42 signaling events | 4/70 | 0.002682635 | 0.035880243 | 0 | 0 | 7.441746659 | 44.06225271 | DLG1; PIK3CA; |
| Homo sapiens 50b98ae0- | | | | | | | | ARHGEF7; ARHGEF6 |
| 6190-11e5-8ac5- | | | | | | | | |
| 06603eb7f303 | | | | | | | | |
| Signaling events | 3/38 | 0.003763346 | 0.038830694 | 0 | 0 | 10.47619048 | 58.48277627 | PIK3CA; IRS2; CRK |
| regulated by Ret tyrosine | | | | | | | | |
| kinase Homo sapiens | | | | | | | | |
| e4431190-6195-11e5- | | | | | | | | |
| 8ac5-06603eb7f303 | | | | | | | | |
| EPHB forward signaling | 3/39 | 0.004052516 | 0.038830694 | 0 | 0 | 10.18467078 | 56.10141609 | PIK3CA; ITSN1; CRK |
| Homo sapiens 01c81f4a- | | | | | | | | |
| 6192-11e5-8ac5- | | | | | | | | |
| 06603eb7f303 | | | | | | | | |
| Plasma membrane | 3/40 | 0.004354844 | 0.038830694 | 0 | 0 | 9.908908909 | 53.86945081 | PIK3CA; ROCK2; |
| estrogen receptor | | | | | | | | GNAI1 |
| signaling Homo sapiens | | | | | | | | |
| dcc37895-6194-11e5- | | | | | | | | |
| 8ac5-06603eb7f303 | | | | | | | | |
| Internalization of ErbB1 | 3/40 | 0.004354844 | 0.038830694 | 0 | 0 | 9.908908909 | 53.86945081 | PIK3CA; ITSN1; |
| Homo sapiens 3aa9aafa- | | | | | | | | ARHGEF7 |
| 6194-11e5-8ac5- | | | | | | | | |
| 06603eb7f303 | | | | | | | | |
| PAR1-mediated thrombin | 3/43 | 0.005342455 | 0.043972518 | 0 | 0 | 9.164351852 | 47.94852957 | PIK3CA; ROCK2; |
| signaling events Homo | | | | | | | | GNAI1 |
| sapiens be5084c0-6194- | | | | | | | | |
| 11e5-8ac5-06603eb7f303 | | | | | | | | |
| Angiopoietin receptor | 3/48 | 0.00726633 | 0.055535522 | 0 | 0 | 8.144032922 | 40.10532209 | ANGPT1; PIK3CA; |
| Tie2-mediated signaling | | | | | | | | CRK |
| Homo sapiens ad60647c- | | | | | | | | |
| 6188-11e5-8ac5- | | | | | | | | |
| 06603eb7f303 | | | | | | | | |
| Signaling events | 3/52 | 0.009064913 | 0.063446922 | 0 | 0 | 7.477702192 | 35.17020598 | PIK3CA; LEP; CRK |
| mediated by PTP1B | | | | | | | | |
| Homo sapiens be498a9b- | | | | | | | | |
| 6195-11e5-8ac5- | | | | | | | | |
| 06603eb7f303 | | | | | | | | |
| CXCR4-mediated | 4/100 | 0.00948739 | 0.063446922 | 0 | 0 | 5.108436853 | 23.79403482 | PIK3CA; ITGA1; CRK; |
| signaling events Homo | | | | | | | | GNAI1 |
| sapiens 46a5529b-6191- | | | | | | | | |
| 11e5-8ac5-06603eb7f303 | | | | | | | | |
| SHP2 signaling Homo | 3/57 | 0.011648548 | 0.073317334 | 0 | 0 | 6.783607682 | 30.20451317 | PIK3CA; ANGPT1; |
| sapiens 85755aa4-6195- | | | | | | | | GNAI1 |
| 11e5-8ac5-06603eb7f303 | | | | | | | | |
| PDGFR-alpha signaling | 2/22 | 0.014023739 | 0.083363335 | 0 | 0 | 12.15644172 | 51.87158259 | PIK3CA; CRK |
| pathway Homo sapiens | | | | | | | | |
| c66cc833-6194-11e5- | | | | | | | | |
| 8ac5-06603eb7f303 | | | | | | | | |
| Integrin family cell | 2/26 | 0.01931096 | 0.108751195 | 0 | 0 | 10.12832311 | 39.97732665 | ITGAM; ITGA1 |
| surface interactions | | | | | | | | |
| Homo sapiens 1ca2bf67- | | | | | | | | |
| 6194-11e5-8ac5- | | | | | | | | |
| 06603eb7f303 | | | | | | | | |
| Nongenotropic Androgen | 2/30 | 0.025299012 | 0.12514128 | 0 | 0 | 8.679666959 | 31.91504809 | PIK3CA; GNAI1 |
| signaling Homo sapiens | | | | | | | | |
| 843e2f77-6194-11e5- | | | | | | | | |
| 8ac5-06603eb7f303 | | | | | | | | |
| Signaling events | 3/77 | 0.02585736 | 0.12514128 | 0 | 0 | 4.945195195 | 18.07547965 | PIK3CA; RANBP9; CRK |
| mediated by Hepatocyte | | | | | | | | |
| Growth Factor Receptor | | | | | | | | |
| (c-Met) Homo sapiens | | | | | | | | |
| ac39d2b9-6195-11e5- | | | | | | | | |
| 8ac5-06603eb7f303 | | | | | | | | |
| Osteopontin-mediated | 2/31 | 0.026899527 | 0.12514128 | 0 | 0 | 8.379944997 | 30.29891928 | PIK3CA; ROCK2 |
| events Homo sapiens | | | | | | | | |
| 94bf7f2a-6194-11e5- | | | | | | | | |
| 8ac5-06603eb7f303 | | | | | | | | |
| Nephrin/Neph1 signaling | 2/31 | 0.026899527 | 0.12514128 | 0 | 0 | 8.379944997 | 30.29891928 | TJP1; PIK3CA |
| in the kidney podocyte | | | | | | | | |
| Homo sapiens 6cfb9873- | | | | | | | | |
| 6194-11e5-8ac5- | | | | | | | | |
| 06603eb7f303 | | | | | | | | |
| N-cadherin signaling | 2/34 | 0.031937157 | 0.136691033 | 0 | 0 | 7.593174847 | 26.15078137 | PIK3CA; LRP5 |
| events Homo sapiens | | | | | | | | |
| 5fc9c1e1-6194-11e5- | | | | | | | | |
| 8ac5-06603eb7f303 | | | | | | | | |
| EGF receptor (ErbB1) | 2/34 | 0.031937157 | 0.136691033 | 0 | 0 | 7.593174847 | 26.15078137 | PIK3CA; GNAI1 |
| signaling pathway Homo | | | | | | | | |
| sapiens NULL | | | | | | | | |
| Regulation of RAC1 | 2/38 | 0.039177187 | 0.161229192 | 0 | 0 | 6.748125426 | 21.86163654 | ARHGEF7; ARHGEF6 |
| activity Homo sapiens | | | | | | | | |
| 351aacd6-6195-11e5- | | | | | | | | |
| 8ac5-06603eb7f303 | | | | | | | | |
| Urokinase-type | 2/42 | 0.046973311 | 0.17331532 | 0 | 0 | 6.07208589 | 18.56950545 | ITGAM; CRK |
| plasminogen activator | | | | | | | | |
| (uPA) and uPAR- | | | | | | | | |
| mediated signaling Homo | | | | | | | | |
| sapiens 503076a2-6196- | | | | | | | | |
| 11e5-8ac5-06603eb7f303 | | | | | | | | |
| Signaling events | 2/42 | 0.046973311 | 0.17331532 | 0 | 0 | 6.07208589 | 18.56950545 | PIK3CA; ITGA1 |
| mediated by TCPTP | | | | | | | | |
| Homo sapiens cd5ca44d- | | | | | | | | |
| 6195-11e5-8ac5- | | | | | | | | |
| 06603eb7f303 | | | | | | | | |
| BMP receptor signaling | 2/42 | 0.046973311 | 0.17331532 | 0 | 0 | 6.07208589 | 18.56950545 | PPM1A; ZFYVE16 |
| Homo sapiens 2a3c66e7- | | | | | | | | |
| 618e-11e5-8ac5- | | | | | | | | |
| 06603eb7f303 | | | | | | | | |
| CXCR3-mediated | 2/43 | 0.049004088 | 0.174781246 | 0 | 0 | 5.923686967 | 17.86496059 | PIK3CA; GNAI1 |
| signaling events Homo | | | | | | | | |
| sapiens 3a38a0ca-6191- | | | | | | | | |
| 11e5-8ac5-06603eb7f303 | | | | | | | | |
| ErbB2/ErbB3 signaling | 2/44 | 0.051066235 | 0.176260875 | 0 | 0 | 5.78235466 | 17.20037584 | PIK3CA; RNF41 |
| events Homo sapiens | | | | | | | | |
| 51e35311-6192-11e5- | | | | | | | | |
| 8ac5-06603eb7f303 | | | | | | | | |
| S1P5 pathway Homo | 1/8 | 0.06413601 | 0.214454783 | 0 | 0 | 17.271777 | 47.44124133 | GNAI1 |
| sapiens 845321c3-6195- | | | | | | | | |
| 11e5-8ac5-06603eb7f303 | | | | | | | | |
| TGF-beta receptor | 2/54 | 0.073274469 | 0.237586914 | 0 | 0 | 4.668003775 | 12.20002878 | ZFYVE16; SPTBN1 |
| signaling Homo sapiens | | | | | | | | |
| 1f188fcc-6196-11e5- | | | | | | | | |
| 8ac5-06603eb7f303 | | | | | | | | |
| IL2-mediated signaling | 2/55 | 0.075640687 | 0.238045692 | 0 | 0 | 4.579696724 | 11.82368216 | PIK3CA; IRS2 |
| events Homo sapiens | | | | | | | | |
| a2a1883c-6193-11e5- | | | | | | | | |
| 8ac5-06603eb7f303 | | | | | | | | |
| Fc-epsilon receptor I | 2/58 | 0.082881874 | 0.246797796 | 0 | 0 | 4.33369851 | 10.79237792 | FCER1G; PIK3CA |
| signaling in mast cells | | | | | | | | |
| Homo sapiens 86cd7795- | | | | | | | | |
| 6192-11e5-8ac5- | | | | | | | | |
| 06603eb7f303 | | | | | | | | |
| FGF signaling pathway | 2/59 | 0.085341294 | 0.246797796 | 0 | 0 | 4.25745345 | 10.47800522 | PIK3CA; FGFR2 |
| Homo sapiens 98ed0df6- | | | | | | | | |
| 6192-11e5-8ac5- | | | | | | | | |
| 06603eb7f303 | | | | | | | | |
| Coregulation of Androgen | 2/59 | 0.085341294 | 0.246797796 | 0 | 0 | 4.25745345 | 10.47800522 | LATS2; NRIP1 |
| receptor activity Homo | | | | | | | | |
| sapiens 27e0e369-6191- | | | | | | | | |
| 11e5-8ac5-06603eb7f303 | | | | | | | | |
| IL4-mediated signaling | 2/60 | 0.087822668 | 0.247290143 | 0 | 0 | 4.183837529 | 10.17691551 | PIK3CA; IRS2 |
| events Homo sapiens | | | | | | | | |
| cff33f50-6193-11e5- | | | | | | | | |
| 8ac5-06603eb7f303 | | | | | | | | |
| Neurotrophic factor- | 2/61 | 0.090325471 | 0.247816035 | 0 | 0 | 4.112717064 | 9.888352838 | PIK3CA; CRK |
| mediated Trk receptor | | | | | | | | |
| signaling Homo sapiens | | | | | | | | |
| 774988f5-6194-11e5- | | | | | | | | |
| 8ac5-06603eb7f303 | | | | | | | | |
| Endothelins Homo | 2/63 | 0.095393303 | 0.255177085 | 0 | 0 | 3.977471588 | 9.346051545 | CRK; GNAI1 |
| sapiens dfb9dc47-6191- | | | | | | | | |
| 11e5-8ac5-06603eb7f303 | | | | | | | | |
| LPA receptor mediated | 2/65 | 0.100540737 | 0.262386801 | 0 | 0 | 3.850813127 | 8.846058235 | CRK; GNAI1 |
| events Homo sapiens | | | | | | | | |
| 4b994cde-6194-11e5- | | | | | | | | |
| 8ac5-06603eb7f303 | | | | | | | | |
| S1P4 pathway Homo | 1/14 | 0.10953953 | 0.26638022 | 0 | 0 | 9.297373358 | 20.56086033 | GNAI1 |
| sapiens 821b0c12-6195- | | | | | | | | |
| 11e5-8ac5-06603eb7f303 | | | | | | | | |
| IL5-mediated signaling | 1/14 | 0.10953953 | 0.26638022 | 0 | 0 | 9.297373358 | 20.56086033 | PIK3CA |
| events Homo sapiens | | | | | | | | |
| e1d816a1-6193-11e5- | | | | | | | | |
| 8ac5-06603eb7f303 | | | | | | | | |
| JNK signaling in the CD4+ | 1/14 | 0.10953953 | 0.26638022 | 0 | 0 | 9.297373358 | 20.56086033 | CRK |
| TCR pathway Homo | | | | | | | | |
| sapiens 400ebdab-6194- | | | | | | | | |
| 11e5-8ac5-06603eb7f303 | | | | | | | | |
| PAR4-mediated thrombin | 1/15 | 0.116890963 | 0.277940734 | 0 | 0 | 8.632839721 | 18.5305089 | ROCK2 |
| signaling events Homo | | | | | | | | |
| sapiens c3b87da1-6194- | | | | | | | | |
| 11e5-8ac5-06603eb7f303 | | | | | | | | |
| Atypical NF-kappaB | 1/17 | 0.131413341 | 0.299175054 | 0 | 0 | 7.552972561 | 15.32806027 | PIK3CA |
| pathway Homo sapiens | | | | | | | | |
| d2dd121d-618b-11e5- | | | | | | | | |
| 8ac5-06603eb7f303 | | | | | | | | |
| Regulation of cytoplasmic | 1/17 | 0.131413341 | 0.299175054 | 0 | 0 | 7.552972561 | 15.32806027 | PPM1A |
| and nuclear SMAD2/3 | | | | | | | | |
| signaling Homo sapiens | | | | | | | | |
| 13312fe2-6195-11e5- | | | | | | | | |
| 8ac5-06603eb7f303 | | | | | | | | |
| Regulation of nuclear | 2/79 | 0.138483374 | 0.30262497 | 0 | 0 | 3.148434388 | 6.224470543 | TCF7L2; CTNNBIP1 |
| beta catenin signaling | | | | | | | | |
| and target gene | | | | | | | | |
| transcription Homo | | | | | | | | |
| sapiens 1590a3b3-6195- | | | | | | | | |
| 11e5-8ac5-06603eb7f303 | | | | | | | | |
| EPHA2 forward signaling | 1/18 | 0.138585266 | 0.30262497 | 0 | 0 | 7.108321377 | 14.04795874 | PIK3CA |
| Homo sapiens fb172ba9- | | | | | | | | |
| 6191-11e5-8ac5- | | | | | | | | |
| 06603eb7f303 | | | | | | | | |
| E-cadherin signaling in | 1/21 | 0.159749784 | 0.328715901 | 0 | 0 | 6.041158537 | 11.08037003 | PIK3CA |
| keratinocytes Homo | | | | | | | | |
| sapiens a5f1af61-6191- | | | | | | | | |
| 11e5-8ac5-06603eb7f303 | | | | | | | | |
| S1P1 pathway Homo | 1/21 | 0.159749784 | 0.328715901 | 0 | 0 | 6.041158537 | 11.08037003 | GNAI1 |
| sapiens 7327884f-6195- | | | | | | | | |
| 11e5-8ac5-06603eb7f303 | | | | | | | | |
| Sphingosine 1-phosphate | 1/21 | 0.159749784 | 0.328715901 | 0 | 0 | 6.041158537 | 11.08037003 | GNAI1 |
| (S1P) pathway Homo | | | | | | | | |
| sapiens eff796f3-6195- | | | | | | | | |
| 11e5-8ac5-06603eb7f303 | | | | | | | | |
| Signaling events | 1/22 | 0.166689124 | 0.336523326 | 0 | 0 | 5.753193961 | 10.30756459 | PIK3CA |
| mediated by the | | | | | | | | |
| Hedgehog family Homo | | | | | | | | |
| sapiens d3a49cee-6195- | | | | | | | | |
| 11e5-8ac5-06603eb7f303 | | | | | | | | |
| Signaling events | 1/23 | 0.1735715 | 0.343928712 | 0 | 0 | 5.491407982 | 9.616365099 | ITGA1 |
| mediated by PRL Homo | | | | | | | | |
| sapiens bb67523a-6195- | | | | | | | | |
| 11e5-8ac5-06603eb7f303 | | | | | | | | |
| Plexin-D1 Signaling Homo | 1/24 | 0.180397375 | 0.344687841 | 0 | 0 | 5.252386002 | 8.995200678 | ITGA1 |
| sapiens e3068f36-6194- | | | | | | | | |
| 11e5-8ac5-06603eb7f303 | | | | | | | | |
| S1P2 pathway Homo | 1/24 | 0.180397375 | 0.344687841 | 0 | 0 | 5.252386002 | 8.995200678 | GNAI1 |
| sapiens 7796a240-6195- | | | | | | | | |
| 11e5-8ac5-06603eb7f303 | | | | | | | | |
| IL3-mediated signaling | 1/26 | 0.193881464 | 0.346478077 | 0 | 0 | 4.831707317 | 7.926456035 | PIK3CA |
| events Homo sapiens | | | | | | | | |
| c868db9f-6193-11e5- | | | | | | | | |
| 8ac5-06603eb7f303 | | | | | | | | |
| VEGFR1 specific signals | 1/27 | 0.200540589 | 0.346478077 | 0 | 0 | 4.645637899 | 7.464325789 | PIK3CA |
| Homo sapiens 8b13143b- | | | | | | | | |
| 6196-11e5-8ac5- | | | | | | | | |
| 06603eb7f303 | | | | | | | | |
| Insulin-mediated glucose | 1/27 | 0.200540589 | 0.346478077 | 0 | 0 | 4.645637899 | 7.464325789 | LNPEP |
| transport Homo sapiens | | | | | | | | |
| 145e3376-6194-11e5- | | | | | | | | |
| 8ac5-06603eb7f303 | | | | | | | | |
| TRAIL signaling pathway | 1/28 | 0.207145036 | 0.346478077 | 0 | 0 | 4.4733514 | 7.04255848 | PIK3CA |
| Homo sapiens 3a79fddf- | | | | | | | | |
| 6196-11e5-8ac5- | | | | | | | | |
| 06603eb7f303 | | | | | | | | |
| Retinoic acid receptors- | 1/28 | 0.207145036 | 0.346478077 | 0 | 0 | 4.4733514 | 7.04255848 | NRIP1 |
| mediated signaling Homo | | | | | | | | |
| sapiens 5797691b-6195- | | | | | | | | |
| 11e5-8ac5-06603eb7f303 | | | | | | | | |
| Wnt signaling network | 1/28 | 0.207145036 | 0.346478077 | 0 | 0 | 4.4733514 | 7.04255848 | LRP5 |
| Homo sapiens 987a2b9f- | | | | | | | | |
| 6196-11e5-8ac5- | | | | | | | | |
| 06603eb7f303 | | | | | | | | |
| Beta2 integrin cell | 1/29 | 0.21369525 | 0.346478077 | 0 | 0 | 4.31337108 | 6.656412982 | ITGAM |
| surface interactions | | | | | | | | |
| Homo sapiens 95b6b434- | | | | | | | | |
| 618d-11e5-8ac5- | | | | | | | | |
| 06603eb7f303 | | | | | | | | |
| Reelin signaling pathway | 1/29 | 0.21369525 | 0.346478077 | 0 | 0 | 4.31337108 | 6.656412982 | PIK3CA |
| Homo sapiens 054f432f- | | | | | | | | |
| 6195-11e5-8ac5- | | | | | | | | |
| 06603eb7f303 | | | | | | | | |
| S1P3 pathway Homo | 1/29 | 0.21369525 | 0.346478077 | 0 | 0 | 4.31337108 | 6.656412982 | GNAI1 |
| sapiens 7cb02d01-6195- | | | | | | | | |
| 11e5-8ac5-06603eb7f303 | | | | | | | | |
| IL2 signaling events | 1/30 | 0.220191675 | 0.346478077 | 0 | 0 | 4.164423886 | 6.30184302 | PIK3CA |
| mediated by STAT5 | | | | | | | | |
| Homo sapiens 9938526b- | | | | | | | | |
| 6193-11e5-8ac5- | | | | | | | | |
| 06603eb7f303 | | | | | | | | |
| Netrin-mediated | 1/30 | 0.220191675 | 0.346478077 | 0 | 0 | 4.164423886 | 6.30184302 | PIK3CA |
| signaling events Homo | | | | | | | | |
| sapiens 716a3d34-6194- | | | | | | | | |
| 11e5-8ac5-06603eb7f303 | | | | | | | | |
| Ephrin B reverse signaling | 1/30 | 0.220191675 | 0.346478077 | 0 | 0 | 4.164423886 | 6.30184302 | PIK3CA |
| Homo sapiens 149a63dc- | | | | | | | | |
| 6192-11e5-8ac5- | | | | | | | | |
| 06603eb7f303 | | | | | | | | |
| Aurora A signaling Homo | 1/31 | 0.226634749 | 0.351448089 | 0 | 0 | 4.025406504 | 5.975376181 | ARHGEF7 |
| sapiens f131cf8e-618b- | | | | | | | | |
| 11e5-8ac5-06603eb7f303 | | | | | | | | |
| Alpha4 beta1 integrin | 1/32 | 0.233024908 | 0.355719395 | 0 | 0 | 3.895357986 | 5.674017125 | CRK |
| signaling events Homo | | | | | | | | |
| sapiens aa07df5d-6187- | | | | | | | | |
| 11e5-8ac5-06603eb7f303 | | | | | | | | |
| IL1-mediated signaling | 1/33 | 0.239362583 | 0.355719395 | 0 | 0 | 3.7734375 | 5.395169587 | PIK3CA |
| events Homo sapiens | | | | | | | | |
| 68fce8e7-6193-11e5- | | | | | | | | |
| 8ac5-06603eb7f303 | | | | | | | | |
| EPO signaling pathway | 1/33 | 0.239362583 | 0.355719395 | 0 | 0 | 3.7734375 | 5.395169587 | IRS2 |
| Homo sapiens 20fe3c0e- | | | | | | | | |
| 6192-11e5-8ac5- | | | | | | | | |
| 06603eb7f303 | | | | | | | | |
| EPHA forward signaling | 1/34 | 0.245648204 | 0.360059697 | 0 | 0 | 3.658906135 | 5.136573052 | CRK |
| Homo sapiens f25420e8- | | | | | | | | |
| 6191-11e5-8ac5- | | | | | | | | |
| 06603eb7f303 | | | | | | | | |
| IL2 signaling events | 1/35 | 0.251882195 | 0.363328324 | 0 | 0 | 3.551111908 | 4.896251013 | PIK3CA |
| mediated by PI3K Homo | | | | | | | | |
| sapiens 8bbf39aa-6193- | | | | | | | | |
| 11e5-8ac5-06603eb7f303 | | | | | | | | |
| GMCSF-mediated | 1/36 | 0.258064978 | 0.363328324 | 0 | 0 | 3.449477352 | 4.672468415 | PIK3CA |
| signaling events Homo | | | | | | | | |
| sapiens 095aa3ef-6193- | | | | | | | | |
| 11e5-8ac5-06603eb7f303 | | | | | | | | |
| Trk receptor signaling | 1/36 | 0.258064978 | 0.363328324 | 0 | 0 | 3.449477352 | 4.672468415 | PIK3CA |
| mediated by PI3K and | | | | | | | | |
| PLC-gamma Homo | | | | | | | | |
| sapiens 4037def0-6196- | | | | | | | | |
| 11e5-8ac5-06603eb7f303 | | | | | | | | |
| IL23-mediated signaling | 1/37 | 0.26419697 | 0.366073529 | 0 | 0 | 3.35348916 | 4.463696472 | PIK3CA |
| events Homo sapiens | | | | | | | | |
| b71d0ffd-6193-11e5- | | | | | | | | |
| 8ac5-06603eb7f303 | | | | | | | | |
| ErbB4 signaling events | 1/38 | 0.270278587 | 0.366073529 | 0 | 0 | 3.262689519 | 4.26858338 | PIK3CA |
| Homo sapiens 6104ebb2- | | | | | | | | |
| 6192-11e5-8ac5- | | | | | | | | |
| 06603eb7f303 | | | | | | | | |
| FAS (CD95) signaling | 1/38 | 0.270278587 | 0.366073529 | 0 | 0 | 3.262689519 | 4.26858338 | PIK3CA |
| pathway Homo sapiens | | | | | | | | |
| 79cc9c14-6192-11e5- | | | | | | | | |
| 8ac5-06603eb7f303 | | | | | | | | |
| amb2 Integrin signaling | 1/40 | 0.282292336 | 0.371851165 | 0 | 0 | 3.095059412 | 3.914668568 | ITGAM |
| Homo sapiens 5d4f90b6- | | | | | | | | |
| 6188-11e5-8ac5- | | | | | | | | |
| 06603eb7f303 | | | | | | | | |
| IFN-gamma pathway | 1/40 | 0.282292336 | 0.371851165 | | 0 | 3.095059412 | 3.914668568 | PIK3CA |
| Homo sapiens 51b1ed75- | | | | | | | | |
| 6193-11e5-8ac5- | | | | 0 | | | | |
| 06603eb7f303 | | | | | | | | |
| PDGFR-beta signaling | 2/128 | 0.284970052 | 0.371851165 | 0 | 0 | 1.919271594 | 2.409398258 | PIK3CA; CRK |
| pathway Homo sapiens | | | | | | | | |
| c901a3e4-6194-11e5- | | | | | | | | |
| 8ac5-06603eb7f303 | | | | | | | | |
| Insulin Pathway Homo | 1/43 | 0.299945319 | 0.381312738 | 0 | 0 | 2.8735482 | 3.46019769 | PIK3CA |
| sapiens 073b9f25-6194- | | | | | | | | |
| 11e5-8ac5-06603eb7f303 | | | | | | | | |
| FOXA1 transcription | 1/44 | 0.305733205 | 0.381312738 | 0 | 0 | 2.806579694 | 3.325916035 | NRIP1 |
| factor network Homo | | | | | | | | |
| sapiens aa3927b7-6192- | | | | | | | | |
| 11e5-8ac5-06603eb7f303 | | | | | | | | |
| a6b1 and a6b4 Integrin | 1/45 | 0.311473526 | 0.381312738 | 0 | 0 | 2.742655211 | 3.199145303 | PIK3CA |
| signaling Homo sapiens | | | | | | | | |
| 73d1a893-6186-11e5- | | | | | | | | |
| Sac5-06603eb7f303 | | | | | | | | |
| RhoA signaling pathway | 1/45 | 0.311473526 | 0.381312738 | 0 | 0 | 2.742655211 | 3.199145303 | ROCK2 |
| Homo sapiens 5c6b5f5c- | | | | | | | | |
| 6195-11e5-8ac5- | | | | | | | | |
| 06603eb7f303 | | | | | | | | |
| TNF receptor signaling | 1/46 | 0.31716667 | 0.381312738 | 0 | 0 | 2.681571816 | 3.079323653 | MAP4K3 |
| pathway Homo sapiens | | | | | | | | |
| 316be05e-6196-11e5- | | | | | | | | |
| 8ac5-06603eb7f303 | | | | | | | | |
| IL6-mediated signaling | 1/46 | 0.31716667 | 0.381312738 | 0 | 0 | 2.681571816 | 3.079323653 | PIK3CA |
| events Homo sapiens | | | | | | | | |
| e684d5d2-6193-11e5- | | | | | | | | |
| 8ac5-06603eb7f303 | | | | | | | | |
| PLK1 signaling events | 1/46 | 0.31716667 | 0.381312738 | 0 | 0 | 2.681571816 | 3.079323653 | ROCK2 |
| Homo sapiens e5e87977- | | | | | | | | |
| 6194-11e5-8ac5- | | | | | | | | |
| 06603eb7f303 | | | | | | | | |
| Hedgehog signaling | 1/47 | 0.322813023 | 0.383788816 | 0 | 0 | 2.623144221 | 2.965941956 | GNAI1 |
| events mediated by Gli | | | | | | | | |
| proteins Homo sapiens | | | | | | | | |
| 153b6970-6193-11e5- | | | | | | | | |
| 8ac5-06603eb7f303 | | | | | | | | |
| Class I PI3K signaling | 1/48 | 0.328412966 | 0.386155905 | 0 | 0 | 2.567202906 | 2.858537872 | PIK3CA |
| events Homo sapiens | | | | | | | | |
| 12b82bb7-6191-11e5- | | | | | | | | |
| 8ac5-06603eb7f303 | | | | | | | | |
| Arf6 trafficking events | 1/49 | 0.333966879 | 0.388418001 | 0 | 0 | 2.51359248 | 2.756690693 | ITGA1 |
| Homo sapiens 7a5b8f09- | | | | | | | | |
| 618a-11e5-8ac5- | | | | | | | | |
| 06603eb7f303 | | | | | | | | |
| Signaling events | 1/52 | 0.350356185 | 0.3988097 | 0 | 0 | 2.365375418 | 2.480817499 | PIK3CA |
| mediated by Stem cell | | | | | | | | |
| factor receptor (c-Kit) | | | | | | | | |
| Homo sapiens c6b6861c- | | | | | | | | |
| 6195-11e5-8ac5- | | | | | | | | |
| 06603eb7f303 | | | | | | | | |
| Regulation of Androgen | 1/52 | 0.350356185 | 0.3988097 | 0 | 0 | 2.365375418 | 2.480817499 | APPBP2 |
| receptor activity Homo | | | | | | | | |
| sapiens 094a8cb0-6195- | | | | | | | | |
| 11e5-8ac5-06603eb7f303 | | | | | | | | |
| RAC1 signaling pathway | 1/54 | 0.361059052 | 0.406666511 | 0 | 0 | 2.275885872 | 2.318476245 | CRK |
| Homo sapiens faffa4fc- | | | | | | | | |
| 6194-11e5-8ac5- | | | | | | | | |
| 06603eb7f303 | | | | | | | | |
| Validated targets of C- | 1/63 | 0.407093659 | 0.445293561 | 0 | 0 | 1.944630212 | 1.747662505 | CSDE1 |
| MYC transcriptional | | | | | | | | |
| repression Homo sapiens | | | | | | | | |
| 6bbdafa6-6196-11e5- | | | | | | | | |
| 8ac5-06603eb7f303 | | | | | | | | |
| BCR signaling pathway | 1/64 | 0.412000584 | 0.445293561 | 0 | 0 | 1.91366628 | 1.696906279 | PIK3CA |
| Homo sapiens acbf44e2- | | | | | | | | |
| 618c-11e5-8ac5- | | | | | | | | |
| 06603eb7f303 | | | | | | | | |
| Validated nuclear | 1/64 | 0.412000584 | 0.445293561 | 0 | 0 | 1.91366628 | 1.696906279 | NRIP1 |
| estrogen receptor alpha | | | | | | | | |
| network Homo sapiens | | | | | | | | |
| 58949883-6196-11e5- | | | | | | | | |
| 8ac5-06603eb7f303 | | | | | | | | |
| Regulation of | 1/64 | 0.412000584 | 0.445293561 | 0 | 0 | 1.91366628 | 1.696906279 | ATF7 |
| retinoblastoma protein | | | | | | | | |
| Homo sapiens 407a3468- | | | | | | | | |
| 6195-11e5-8ac5- | | | | | | | | |
| 06603eb7f303 | | | | | | | | |
| Beta1 integrin cell | 1/66 | 0.421693666 | 0.446744775 | 0 | 0 | 1.854596623 | 1.60139993 | ITGA1 |
| surface interactions | | | | | | | | |
| Homo sapiens 2fd0bc63- | | | | | | | | |
| 618d-11e5-8ac5- | | | | | | | | |
| 06603eb7f303 | | | | | | | | |
| HIF-1-alpha transcription | 1/66 | 0.421693666 | 0.446744775 | 0 | 0 | 1.854596623 | 1.60139993 | LEP |
| factor network Homo | | | | | | | | |
| sapiens 20ef2b81-6193- | | | | | | | | |
| 11e5-8ac5-06603eb7f303 | | | | | | | | |
| p75(NTR)-mediated | 1/68 | 0.431227911 | 0.447974626 | 0 | 0 | 1.799053513 | 1.513217252 | PIK3CA |
| signaling Homo sapiens | | | | | | | | |
| b492782f-6194-11e5- | | | | | | | | |
| 8ac5-06603eb7f303 | | | | | | | | |
| Signaling events | 1/68 | 0.431227911 | 0.447974626 | 0 | 0 | 1.799053513 | 1.513217252 | PIK3CA |
| mediated by VEGFR1 and | | | | | | | | |
| VEGFR2 Homo sapiens | | | | | | | | |
| d6f6ae1f-6195-11e5- | | | | | | | | |
| 8ac5-06603eb7f303 | | | | | | | | |
| AP-1 transcription factor | 1/69 | 0.43593628 | 0.448511365 | 0 | 0 | 1.772507174 | 1.471640377 | TCF7L2 |
| network Homo sapiens | | | | | | | | |
| 3ce2f9c5-6189-11e5- | | | | | | | | |
| 8ac5-06603eb7f303 | | | | | | | | |
| Integrins in angiogenesis | 1/72 | 0.449830197 | 0.458398391 | 0 | 0 | 1.697354861 | 1.35599152 | PIK3CA |
| Homo sapiens 2ddeac89- | | | | | | | | |
| 6194-11e5-8ac5- | | | | | | | | |
| 06603eb7f303 | | | | | | | | |
| Glucocorticoid receptor | 1/82 | 0.493732324 | 0.498390176 | 0 | 0 | 1.487052093 | 1.049504508 | VIPR1 |
| regulatory network | | | | | | | | |
| Homo sapiens dfba0dfb- | | | | | | | | |
| 6192-11e5-8ac5- | | | | | | | | |
| 06603eb7f303 | | | | | | | | |
| ErbB1 downstream | 1/105 | 0.581935755 | 0.581935755 | 0 | 0 | 1.156836304 | 0.62630565 | PIK3CA |
| signaling Homo sapiens | | | | | | | | |
| 30d60550-6192-11e5- | | | | | | | | |
| 8ac5-06603eb7f303 | | | | | | | | |

Term, which pathway; Overlap, number of genes that overlap and total genes; P-value, enrichment p-value; Adjusted P-value, Q-value; Odds Ratio, enrichment; Combined Score, approximation of overall association (−log10(P) * log(Odds)), Genes, genes in the pathway which are co-expressed with COBLL1.

TABLE 14
Differences in morphological profiles of siCOBLL-and siNT-treated AMSCs at
day 14 of differentiation. AP features, Adipocyte Profiler feature name; pvalue,
q-value, t-statistics of comparison.

| AP features | pvalue | q-value | t-statistics |
|---|---|---|---|
| Cells_AreaShape_Area | 0.0243 | 0.0360 | 4.2101 |
| Cells_AreaShape_Extent | 0.0332 | 0.0396 | −3.3896 |
| Cells_AreaShape_MajorAxisLength | 0.0249 | 0.0366 | 3.5869 |
| Cells_AreaShape_MaxFeretDiameter | 0.0121 | 0.0253 | 4.7060 |
| Cells_AreaShape_Perimeter | 0.0109 | 0.0253 | 7.1338 |
| Cells_AreaShape_Zernike_4_4 | 0.0342 | 0.0404 | −3.5356 |
| Cells_Children_LargeBODIPYObjects_Count | 0.0064 | 0.0253 | −7.3667 |
| Cells_Correlation_Correlation_BODIPY_AGP | 0.0062 | 0.0253 | 5.6509 |
| Cells_Correlation_Correlation_Mito_AGP | 0.0350 | 0.0407 | 3.7886 |
| Cells_Correlation_Correlation_Mito_BODIPY | 0.0138 | 0.0262 | 4.3484 |
| Cells_Correlation_K_Mito_AGP | 0.0319 | 0.0392 | 3.3510 |
| Cells_Correlation_K_Mito_BODIPY | 0.0264 | 0.0375 | 4.0663 |
| Cells_Granularity_11_BODIPY | 0.0486 | 0.0491 | 2.8876 |
| Cells_Granularity_3_BODIPY | 0.0484 | 0.0489 | −3.5220 |
| Cells_Granularity_8_Mito | 0.0448 | 0.0468 | 2.9000 |
| Cells_Intensity_IntegratedIntensity_AGP | 0.0102 | 0.0253 | 7.9337 |
| Cells_Intensity_IntegratedIntensityEdge_AGP | 0.0127 | 0.0253 | 8.5298 |
| Cells_Intensity_IntegratedIntensityEdge_BODIPY | 0.0407 | 0.0442 | −3.3538 |
| Cells_Intensity_LowerQuartileIntensity_AGP | 0.0028 | 0.0253 | 6.8475 |
| Cells_Intensity_LowerQuartileIntensity_BODIPY | 0.0115 | 0.0253 | −4.4546 |
| Cells_Intensity_MADIntensity_AGP | 0.0149 | 0.0271 | 4.7567 |
| Cells_Intensity_MassDisplacement_BODIPY | 0.0019 | 0.0214 | 11.2167 |
| Cells_Intensity_MassDisplacement_Mito | 0.0002 | 0.0147 | 21.7339 |
| Cells_Intensity_MaxIntensity_AGP | 0.0128 | 0.0253 | 4.6331 |
| Cells_Intensity_MaxIntensityEdge_AGP | 0.0037 | 0.0253 | 9.3496 |
| Cells_Intensity_MeanIntensity_AGP | 0.0032 | 0.0253 | 6.4003 |
| Cells_Intensity_MeanIntensityEdge_AGP | 0.0104 | 0.0253 | 8.1166 |
| Cells_Intensity_MedianIntensity_AGP | 0.0031 | 0.0253 | 6.4014 |
| Cells_Intensity_MinIntensity_AGP | 0.0008 | 0.0164 | 9.1612 |
| Cells_Intensity_MinIntensityEdge_AGP | 0.0007 | 0.0164 | 10.2839 |
| Cells_Intensity_MinIntensityEdge_BODIPY | 0.0117 | 0.0253 | −4.4907 |
| Cells_Intensity_StdIntensity_AGP | 0.0122 | 0.0253 | 5.2030 |
| Cells_Intensity_StdIntensityEdge_AGP | 0.0047 | 0.0253 | 6.9709 |
| Cells_Intensity_UpperQuartileIntensity_AGP | 0.0041 | 0.0253 | 6.1030 |
| Cells_Mean_LargeBODIPYObjects_AreaShape_Center_X | 0.0170 | 0.0291 | −5.0200 |
| Cells_Mean_LargeBODIPYObjects_AreaShape_Center_Z | 0.0088 | 0.0253 | −5.0592 |
| Cells_Mean_LargeBODIPYObjects_AreaShape_Compactness | 0.0088 | 0.0253 | −5.5559 |
| Cells_Mean_LargeBODIPYObjects_AreaShape_Eccentricity | 0.0073 | 0.0253 | −5.2952 |
| Cells_Mean_LargeBODIPYObjects_AreaShape_EulerNumber | 0.0087 | 0.0253 | −5.0830 |
| Cells_Mean_LargeBODIPYObjects_AreaShape_Extent | 0.0129 | 0.0253 | −4.5815 |
| Cells_Mean_LargeBODIPYObjects_AreaShape_FormFactor | 0.0207 | 0.0323 | −4.1719 |
| Cells_Mean_LargeBODIPYObjects_AreaShape_Solidity | 0.0108 | 0.0253 | −4.7739 |
| Cells_Mean_LargeBODIPYObjects_Correlation_Correlation_BODIPY_AGP | 0.0000 | 0.0060 | 45.0534 |
| Cells_Mean_LargeBODIPYObjects_Correlation_Correlation_Mito_AGP | 0.0029 | 0.0253 | −6.9109 |
| Cells_Mean_LargeBODIPYObjects_Correlation_Correlation_Mito_BODIPY | 0.0004 | 0.0164 | 10.7637 |
| Cells_Mean_LargeBODIPYObjects_Correlation_K_AGP_BODIPY | 0.0302 | 0.0386 | −3.4324 |
| Cells_Mean_LargeBODIPYObjects_Correlation_K_Mito_AGP | 0.0260 | 0.0374 | 3.4761 |
| Cells_Mean_LargeBODIPYObjects_Correlation_K_Mito_BODIPY | 0.0279 | 0.0381 | 3.4429 |
| Cells_Mean_LargeBODIPYObjects_Correlation_Overlap_BODIPY_AGP | 0.0168 | 0.0290 | −4.1035 |
| Cells_Mean_LargeBODIPYObjects_Correlation_Overlap_Mito_AGP | 0.0098 | 0.0253 | −4.8818 |
| Cells_Mean_LargeBODIPYObjects_Correlation_Overlap_Mito_BODIPY | 0.0161 | 0.0283 | −4.2111 |
| Cells_Mean_LargeBODIPYObjects_Granularity_10_BODIPY | 0.0208 | 0.0323 | 5.2476 |
| Cells_Mean_LargeBODIPYObjects_Granularity_3_BODIPY | 0.0045 | 0.0253 | −5.7960 |
| Cells_Mean_LargeBODIPYObjects_Intensity_MinIntensity_BODIPY | 0.0176 | 0.0297 | −4.0559 |
| Cells_Mean_LargeBODIPYObjects_Intensity_MinIntensityEdge_BODIPY | 0.0177 | 0.0297 | −4.0507 |
| Cells_Mean_LargeBODIPYObjects_Location_Center_X | 0.0170 | 0.0291 | −5.0212 |
| Cells_Mean_LargeBODIPYObjects_Location_CenterMassIntensity_X_BODIPY | 0.0170 | 0.0291 | −5.0216 |
| Cells_Mean_LargeBODIPYObjects_Location_MaxIntensity_X_BODIPY | 0.0171 | 0.0291 | −5.0256 |
| Cells_Mean_LargeBODIPYObjects_Number_Object_Number | 0.0012 | 0.0177 | −8.3021 |
| Cells_Neighbors_FirstClosestDistance_10 | 0.0110 | 0.0253 | 5.1159 |
| Cells_Neighbors_FirstClosestDistance_Adjacent | 0.0110 | 0.0253 | 5.1159 |
| Cells_Neighbors_FirstClosestObjectNumber_10 | 0.0139 | 0.0262 | −4.5929 |
| Cells_Neighbors_FirstClosestObjectNumber_Adjacent | 0.0139 | 0.0262 | −4.5929 |
| Cells_Neighbors_SecondClosestDistance_10 | 0.0271 | 0.0378 | 4.6651 |
| Cells_Neighbors_SecondClosestDistance_Adjacent | 0.0271 | 0.0378 | 4.6651 |
| Cells_Neighbors_SecondClosestObjectNumber_10 | 0.0126 | 0.0253 | −4.6259 |
| Cells_Neighbors_SecondClosestObjectNumber_Adjacent | 0.0126 | 0.0253 | −4.6259 |
| Cells_Number_Object_Number | 0.0127 | 0.0253 | −4.6547 |
| Cells_RadialDistribution_FracAtD_Mito_1of4 | 0.0294 | 0.0386 | 3.6724 |
| Cells_RadialDistribution_MeanFrac_AGP_3of4 | 0.0449 | 0.0468 | 2.8836 |
| Cells_RadialDistribution_MeanFrac_Mito_4of4 | 0.0195 | 0.0316 | −4.6966 |
| Cells_RadialDistribution_RadialCV_AGP_4of4 | 0.0262 | 0.0375 | −3.7993 |
| Cells_RadialDistribution_RadialCV_BODIPY_2of4 | 0.0376 | 0.0423 | 3.0617 |
| Cells_RadialDistribution_RadialCV_Mito_2of4 | 0.0051 | 0.0253 | 5.5567 |
| Cells_Texture_AngularSecondMoment_AGP_20_00 | 0.0421 | 0.0451 | −2.9748 |
| Cells_Texture_AngularSecondMoment_Mito_10_01 | 0.0376 | 0.0423 | 4.6736 |
| Cells_Texture_Contrast_AGP_10_00 | 0.0448 | 0.0468 | 3.1024 |
| Cells_Texture_Correlation_AGP_20_01 | 0.0272 | 0.0378 | 4.7706 |
| Cells_Texture_Correlation_BODIPY_10_01 | 0.0388 | 0.0431 | 4.2045 |
| Cells_Texture_Correlation_Mito_5_02 | 0.0059 | 0.0253 | 5.8347 |
| Cells_Texture_DifferenceEntropy_AGP_5_00 | 0.0357 | 0.0411 | 3.3332 |
| Cells_Texture_DifferenceEntropy_Mito_20_01 | 0.0460 | 0.0475 | −3.1953 |
| Cells_Texture_DifferenceVariance_Mito_10_01 | 0.0395 | 0.0436 | 4.7080 |
| Cells_Texture_Entropy_AGP_5_01 | 0.0182 | 0.0300 | 4.3073 |
| Cells_Texture_Entropy_Mito_20_00 | 0.0403 | 0.0439 | −3.4410 |
| Cells_Texture_InfoMeas1_AGP_20_00 | 0.0363 | 0.0413 | −3.8141 |
| Cells_Texture_InfoMeas1_BODIPY_5_02 | 0.0399 | 0.0437 | −3.0024 |
| Cells_Texture_InfoMeas1_Mito_10_01 | 0.0206 | 0.0323 | −4.7398 |
| Cells_Texture_InverseDifferenceMoment_AGP_20_03 | 0.0471 | 0.0481 | −3.6963 |
| Cells_Texture_InverseDifferenceMoment_Mito_20_01 | 0.0208 | 0.0323 | 3.7009 |
| Cells_Texture_SumAverage_AGP_20_03 | 0.0128 | 0.0253 | 5.6297 |
| Cells_Texture_SumEntropy_AGP_5_01 | 0.0159 | 0.0282 | 5.1481 |
| Cells_Texture_SumEntropy_Mito_20_03 | 0.0466 | 0.0479 | −3.2644 |
| Cells_Texture_SumVariance_AGP_5_02 | 0.0096 | 0.0253 | 6.0441 |
| Cells_Texture_Variance_AGP_5_02 | 0.0122 | 0.0253 | 5.3694 |
| Cytoplasm_AreaShape_Area | 0.0261 | 0.0374 | 4.1195 |
| Cytoplasm_AreaShape_Compactness | 0.0153 | 0.0276 | 4.2984 |
| Cytoplasm_AreaShape_Extent | 0.0166 | 0.0290 | −4.1935 |
| Cytoplasm_AreaShape_FormFactor | 0.0064 | 0.0253 | −7.7320 |
| Cytoplasm_AreaShape_MajorAxisLength | 0.0241 | 0.0359 | 3.6067 |
| Cytoplasm_AreaShape_MaxFeretDiameter | 0.0121 | 0.0253 | 4.7060 |
| Cytoplasm_AreaShape_Perimeter | 0.0081 | 0.0253 | 7.2861 |
| Cytoplasm_AreaShape_Zernike_8_8 | 0.0483 | 0.0489 | −2.8355 |
| Cytoplasm_Correlation_Correlation_BODIPY_AGP | 0.0055 | 0.0253 | 5.8211 |
| Cytoplasm_Correlation_Correlation_Mito_BODIPY | 0.0090 | 0.0253 | 4.9344 |
| Cytoplasm_Correlation_K_Mito_AGP | 0.0299 | 0.0386 | 3.4021 |
| Cytoplasm_Correlation_K_Mito_BODIPY | 0.0258 | 0.0373 | 4.0678 |
| Cytoplasm_Granularity_11_BODIPY | 0.0471 | 0.0481 | 2.9069 |
| Cytoplasm_Granularity_9_BODIPY | 0.0458 | 0.0475 | 3.8191 |
| Cytoplasm_Granularity_9_Mito | 0.0292 | 0.0386 | 5.3502 |
| Cytoplasm_Intensity_IntegratedIntensity_AGP | 0.0113 | 0.0253 | 7.7759 |
| Cytoplasm_Intensity_IntegratedIntensityEdge_AGP | 0.0111 | 0.0253 | 8.6662 |
| Cytoplasm_Intensity_IntegratedIntensityEdge_BODIPY | 0.0407 | 0.0442 | −3.3431 |
| Cytoplasm_Intensity_LowerQuartileIntensity_AGP | 0.0031 | 0.0253 | 6.9773 |
| Cytoplasm_Intensity_LowerQuartileIntensity_BODIPY | 0.0105 | 0.0253 | −4.5561 |
| Cytoplasm_Intensity_MADIntensity_AGP | 0.0127 | 0.0253 | 4.7144 |
| Cytoplasm_Intensity_MassDisplacement_BODIPY | 0.0017 | 0.0206 | 11.3823 |
| Cytoplasm_Intensity_MassDisplacement_Mito | 0.0003 | 0.0147 | 21.6648 |
| Cytoplasm_Intensity_MaxIntensity_AGP | 0.0126 | 0.0253 | 4.6396 |
| Cytoplasm_Intensity_MaxIntensityEdge_AGP | 0.0058 | 0.0253 | 5.3742 |
| Cytoplasm_Intensity_MeanIntensity_AGP | 0.0029 | 0.0253 | 6.4794 |
| Cytoplasm_Intensity_MeanIntensityEdge_AGP | 0.0059 | 0.0253 | 8.2149 |
| Cytoplasm_Intensity_MedianIntensity_AGP | 0.0026 | 0.0253 | 6.6893 |
| Cytoplasm_Intensity_MinIntensity_AGP | 0.0008 | 0.0164 | 9.1879 |
| Cytoplasm_Intensity_MinIntensity_BODIPY | 0.0201 | 0.0323 | −3.7593 |
| Cytoplasm_Intensity_MinIntensityEdge_AGP | 0.0007 | 0.0164 | 10.2880 |
| Cytoplasm_Intensity_MinIntensityEdge_BODIPY | 0.0188 | 0.0308 | −3.8421 |
| Cytoplasm_Intensity_StdIntensity_AGP | 0.0115 | 0.0253 | 5.1045 |
| Cytoplasm_Intensity_StdIntensityEdge_AGP | 0.0074 | 0.0253 | 5.0802 |
| Cytoplasm_Intensity_UpperQuartileIntensity_AGP | 0.0037 | 0.0253 | 6.0958 |
| Cytoplasm_Number_Object_Number | 0.0127 | 0.0253 | −4.6547 |
| Cytoplasm_Parent_Cells | 0.0127 | 0.0253 | −4.6547 |
| Cytoplasm_RadialDistribution_FracAtD_Mito_1of4 | 0.0233 | 0.0348 | −3.6518 |
| Cytoplasm_RadialDistribution_RadialCV_AGP_1of4 | 0.0001 | 0.0101 | −14.9563 |
| Cytoplasm_RadialDistribution_RadialCV_BODIPY_2of4 | 0.0156 | 0.0279 | 4.1361 |
| Cytoplasm_RadialDistribution_RadialCV_Mito_4of4 | 0.0017 | 0.0208 | 7.6019 |
| Cytoplasm_Texture_AngularSecondMoment_Mito_10_01 | 0.0309 | 0.0390 | 5.1197 |
| Cytoplasm_Texture_Contrast_AGP_5_02 | 0.0466 | 0.0479 | 3.0394 |
| Cytoplasm_Texture_Correlation_AGP_20_01 | 0.0325 | 0.0392 | 3.7190 |
| Cytoplasm_Texture_Correlation_BODIPY_5_01 | 0.0466 | 0.0479 | 2.9766 |
| Cytoplasm_Texture_Correlation_Mito_5_02 | 0.0125 | 0.0253 | 5.3732 |
| Cytoplasm_Texture_DifferenceEntropy_AGP_5_00 | 0.0297 | 0.0386 | 3.4155 |
| Cytoplasm_Texture_DifferenceEntropy_Mito_20_01 | 0.0408 | 0.0442 | −3.3896 |
| Cytoplasm_Texture_DifferenceVariance_Mito_10_01 | 0.0360 | 0.0412 | 4.9768 |
| Cytoplasm_Texture_Entropy_AGP_5_01 | 0.0325 | 0.0392 | 4.0343 |
| Cytoplasm_Texture_Entropy_Mito_20_00 | 0.0346 | 0.0404 | −3.6911 |
| Cytoplasm_Texture_InfoMeas1_AGP_20_03 | 0.0488 | 0.0492 | −3.8374 |
| Cytoplasm_Texture_InfoMeas1_BODIPY_5_02 | 0.0417 | 0.0449 | −2.9574 |
| Cytoplasm_Texture_InfoMeas1_Mito_10_02 | 0.0142 | 0.0265 | −5.3994 |
| Cytoplasm_Texture_InverseDifferenceMoment_AGP_20_01 | 0.0432 | 0.0457 | −3.7076 |
| Cytoplasm_Texture_InverseDifferenceMoment_Mito_20_01 | 0.0178 | 0.0297 | 3.8919 |
| Cytoplasm_Texture_SumAverage_AGP_20_03 | 0.0142 | 0.0265 | 5.4477 |
| Cytoplasm_Texture_SumEntropy_AGP_5_01 | 0.0294 | 0.0386 | 4.7114 |
| Cytoplasm_Texture_SumEntropy_Mito_10_03 | 0.0493 | 0.0495 | −3.1907 |
| Cytoplasm_Texture_SumVariance_AGP_5_02 | 0.0110 | 0.0253 | 5.6227 |
| Cytoplasm_Texture_Variance_AGP_5_02 | 0.0143 | 0.0266 | 5.0206 |

TABLE 15
Differences in morphological profiles between TT (n = 7) and CC (n = 6) allele carriers
at day 14 in subcutaneous AMSCs. Results of multi-way ANOVA with significance level 5%
FDR. AP features, Adipocyte Profiler feature name; pvalue, q-value, t-statistics of comparison.

| | | | effect size | |
|---|---|---|---|---|
| AP features | pvalue | q-value | (eta_sq) | t-test |
| Cells_AreaShape_FormFactor | 0.0389 | 0.0395 | 0.3000 | 1.9862 |
| Cells_AreaShape_Solidity | 0.0136 | 0.0313 | 0.4180 | 2.8491 |
| Cells_AreaShape_Zernike_8_8 | 0.0170 | 0.0326 | 0.3550 | 2.6712 |
| Cells_Children_LargeBODIPYObjects_Count | 0.0186 | 0.0339 | 0.4990 | 2.3018 |
| Cells_Granularity_14_AGP | 0.0123 | 0.0304 | 0.2630 | 1.9214 |
| Cells_Granularity_14_BODIPY | 0.0042 | 0.0298 | 0.5370 | 1.5891 |
| Cells_Granularity_7_AGP | 0.0257 | 0.0365 | 0.3810 | −2.0191 |
| Cells_Granularity_5_BODIPY | 0.0491 | 0.0425 | 0.2730 | 2.1404 |
| Cells_Granularity_8_Mito | 0.0213 | 0.0349 | 0.1120 | −1.1109 |
| Cells_Intensity_LowerQuartileIntensity_Mito | 0.0206 | 0.0348 | 0.3700 | 2.1866 |
| Cells_Intensity_MADIntensity_AGP | 0.0017 | 0.0298 | 0.0580 | −1.1573 |
| Cells_Intensity_MassDisplacement_AGP | 0.0209 | 0.0349 | 0.2720 | −1.7026 |
| Cells_Intensity_MassDisplacement_BODIPY | 0.0028 | 0.0298 | 0.3480 | −2.5211 |
| Cells_Intensity_MassDisplacement_Mito | 0.0297 | 0.0374 | 0.2040 | −1.3490 |
| Cells_Intensity_MaxIntensity_AGP | 0.0242 | 0.0365 | 0.0680 | −1.1854 |
| Cells_Intensity_MeanIntensityEdge_Mito | 0.0454 | 0.0412 | 0.3330 | 2.0549 |
| Cells_Intensity_MedianIntensity_Mito | 0.0395 | 0.0396 | 0.3120 | 2.0166 |
| Cells_Intensity_MinIntensity_Mito | 0.0259 | 0.0365 | 0.4250 | 2.7873 |
| Cells_Intensity_MinIntensityEdge_Mito | 0.0264 | 0.0366 | 0.4220 | 2.7584 |
| Cells_Intensity_StdIntensity_AGP | 0.0128 | 0.0304 | 0.0960 | −1.3545 |
| Cells_Mean_LargeBODIPYObjects_AreaShape_Orientation | 0.0020 | 0.0298 | 0.2580 | 1.7141 |
| Cells_Mean_LargeBODIPYObjects_Correlation_Overlap_BODIPY_AGP | 0.0277 | 0.0374 | 0.2690 | 2.3174 |
| Cells_Mean_LargeBODIPYObjects_Granularity_5_BODIPY | 0.0482 | 0.0423 | 0.4710 | 2.5675 |
| Cells_Neighbors_AngleBetweenNeighbors_10 | 0.0296 | 0.0374 | 0.3170 | 2.2575 |
| Cells_Neighbors_AngleBetweenNeighbors_Adjacent | 0.0296 | 0.0374 | 0.3170 | 2.2575 |
| Cells_RadialDistribution_MeanFrac_AGP_1of4 | 0.0118 | 0.0304 | 0.5930 | −3.7736 |
| Cells_RadialDistribution_MeanFrac_Mito_3of4 | 0.0326 | 0.0375 | 0.3170 | −1.9113 |
| Cells_RadialDistribution_RadialCV_BODIPY_3of4 | 0.0070 | 0.0298 | 0.2500 | −2.1039 |
| Cells_RadialDistribution_RadialCV_Mito_1of4 | 0.0474 | 0.0418 | 0.2080 | 1.7808 |
| Cells_Texture_AngularSecondMoment_AGP_20_03 | 0.0360 | 0.0384 | 0.0460 | 0.9478 |
| Cells_Texture_AngularSecondMoment_Mito_20_03 | 0.0362 | 0.0384 | 0.2800 | −1.8302 |
| Cells_Texture_Contrast_AGP_20_01 | 0.0146 | 0.0313 | 0.0940 | −1.3760 |
| Cells_Texture_Correlation_AGP_20_03 | 0.0289 | 0.0374 | 0.4880 | −2.2589 |
| Cells_Texture_Correlation_Mito_20_00 | 0.0141 | 0.0313 | 0.2810 | −1.9104 |
| Cells_Texture_DifferenceEntropy_AGP_20_01 | 0.0170 | 0.0326 | 0.0730 | −1.1987 |
| Cells_Texture_DifferenceVariance_AGP_20_01 | 0.0157 | 0.0321 | 0.0670 | 1.1178 |
| Cells_Texture_Entropy_AGP_20_03 | 0.0022 | 0.0298 | 0.0680 | −1.1877 |
| Cells_Texture_InverseDifferenceMoment_AGP_5_00 | 0.0099 | 0.0298 | 0.0370 | 0.9324 |
| Cells_Texture_SumEntropy_AGP_20_00 | 0.0014 | 0.0298 | 0.1060 | −1.4187 |
| Cells_Texture_SumEntropy_BODIPY_20_03 | 0.0432 | 0.0403 | 0.4170 | 2.0075 |
| Cells_Texture_SumVariance_AGP_20_00 | 0.0045 | 0.0298 | 0.1530 | −1.6931 |
| Cells_Texture_Variance_AGP_20_01 | 0.0063 | 0.0298 | 0.1240 | −1.5378 |
| Cytoplasm_AreaShape_FormFactor | 0.0330 | 0.0375 | 0.2970 | 2.3550 |
| Cytoplasm_AreaShape_Solidity | 0.0249 | 0.0365 | 0.3410 | 2.8358 |
| Cytoplasm_AreaShape_Zernike_8_8 | 0.0182 | 0.0336 | 0.3490 | 2.6257 |
| Cytoplasm_Granularity_14_AGP | 0.0176 | 0.0332 | 0.2390 | 1.8178 |
| Cytoplasm_Granularity_14_BODIPY | 0.0060 | 0.0298 | 0.5260 | 1.5386 |
| Cytoplasm_Granularity_7_AGP | 0.0286 | 0.0374 | 0.3410 | −1.7705 |
| Cytoplasm_Granularity_5_BODIPY | 0.0245 | 0.0365 | 0.3810 | 2.6321 |
| Cytoplasm_Granularity_8_Mito | 0.0183 | 0.0336 | 0.0900 | −0.9719 |
| Cytoplasm_Intensity_LowerQuartileIntensity_Mito | 0.0202 | 0.0348 | 0.3820 | 2.2758 |
| Cytoplasm_Intensity_MADIntensity_AGP | 0.0023 | 0.0298 | 0.0420 | −1.0256 |
| Cytoplasm_Intensity_MassDisplacement_AGP | 0.0431 | 0.0403 | 0.2490 | −1.6158 |
| Cytoplasm_Intensity_MassDisplacement_BODIPY | 0.0120 | 0.0304 | 0.3150 | −2.5346 |
| Cytoplasm_Intensity_MassDisplacement_Mito | 0.0190 | 0.0340 | 0.2350 | −1.5019 |
| Cytoplasm_Intensity_MaxIntensity_AGP | 0.0422 | 0.0403 | 0.0650 | −1.1326 |
| Cytoplasm_Intensity_MaxIntensityEdge_AGP | 0.0286 | 0.0374 | 0.1000 | −1.3495 |
| Cytoplasm_Intensity_MedianIntensity_Mito | 0.0347 | 0.0383 | 0.3310 | 2.0946 |
| Cytoplasm_Intensity_MinIntensity_Mito | 0.0259 | 0.0365 | 0.4250 | 2.7874 |
| Cytoplasm_Intensity_MinIntensityEdge_Mito | 0.0264 | 0.0366 | 0.4220 | 2.7587 |
| Cytoplasm_Intensity_StdIntensity_AGP | 0.0195 | 0.0346 | 0.0920 | −1.3041 |
| Cytoplasm_Intensity_StdIntensityEdge_AGP | 0.0129 | 0.0304 | 0.1040 | −1.4024 |
| Cytoplasm_RadialDistribution_FracAtD_AGP_3of4 | 0.0152 | 0.0317 | 0.3610 | 2.9312 |
| Cytoplasm_RadialDistribution_FracAtD_BODIPY_1of4 | 0.0030 | 0.0298 | 0.4720 | 3.3039 |
| Cytoplasm_RadialDistribution_MeanFrac_BODIPY_1of4 | 0.0023 | 0.0298 | 0.4540 | 3.1744 |
| Cytoplasm_Texture_AngularSecondMoment_AGP_20_03 | 0.0148 | 0.0313 | 0.0380 | 0.8972 |
| Cytoplasm_Texture_AngularSecondMoment_Mito_20_03 | 0.0313 | 0.0374 | 0.3060 | −1.9246 |
| Cytoplasm_Texture_Contrast_AGP_20_01 | 0.0190 | 0.0340 | 0.0910 | −1.3224 |
| Cytoplasm_Texture_Correlation_Mito_20_03 | 0.0321 | 0.0374 | 0.3750 | −2.1269 |
| Cytoplasm_Texture_DifferenceEntropy_AGP_20_01 | 0.0255 | 0.0365 | 0.0740 | −1.1954 |
| Cytoplasm_Texture_DifferenceVariance_AGP_5_02 | 0.0220 | 0.0358 | 0.0700 | 1.1405 |
| Cytoplasm_Texture_Entropy_AGP_20_01 | 0.0068 | 0.0298 | 0.0580 | −1.1054 |
| Cytoplasm_Texture_InverseDifferenceMoment_AGP_5_00 | 0.0049 | 0.0298 | 0.0410 | 0.9550 |
| Cytoplasm_Texture_InverseDifferenceMoment_Mito_20_03 | 0.0473 | 0.0418 | 0.2710 | −1.7694 |
| Cytoplasm_Texture_SumEntropy_AGP_20_03 | 0.0075 | 0.0298 | 0.0900 | −1.3071 |
| Cytoplasm_Texture_SumVariance_AGP_20_03 | 0.0107 | 0.0298 | 0.1240 | −1.5018 |
| Cytoplasm_Texture_Variance_AGP_20_01 | 0.0122 | 0.0304 | 0.1100 | −1.4230 |

TABLE 16
rs12454712-mediated LipocyteProfiler in subcutaneous AMSCs at day8. (ANOVA
adj. BMI, sex, age, batch, significance level 5% FDR). P-value, p-value of ANOVA, q-value,
q-value of ANOVA, FDR; eta_sq, eta square of ANOVA, effect size; F value of ANOVA;
t-statistics of t-test.

| Lipocyte Profiler features | p-value | q-value | eta_sq | t-statistics |
|---|---|---|---|---|
| Cells_Correlation_K_AGP_DNA | 4.11E−02 | 2.74E−02 | 0.28 | 2.47 |
| Cells_Correlation_K_AGP_Mito | 9.28E−03 | 1.64E−02 | 0.43 | 3.31 |
| Cells_Correlation_K_BODIPY_AGP | 1.64E−02 | 2.29E−02 | 0.40 | −2.15 |
| Cells_Correlation_K_DNA_AGP | 2.18E−02 | 2.30E−02 | 0.35 | −2.33 |
| Cells_Correlation_K_Mito_AGP | 6.52E−04 | 1.28E−02 | 0.56 | −2.93 |
| Cells_Correlation_K_Mito_DNA | 2.29E−02 | 2.31E−02 | 0.28 | −1.55 |
| Cells_Correlation_Overlap_BODIPY_AGP | 4.95E−02 | 2.87E−02 | 0.26 | −1.70 |
| Cells_Correlation_Overlap_DNA_Mito | 6.61E−03 | 1.33E−02 | 0.50 | −3.97 |
| Cells_Correlation_Overlap_Mito_BODIPY | 3.47E−02 | 2.71E−02 | 0.30 | −1.80 |
| Cells_Granularity_1_Mito | 8.23E−03 | 1.50E−02 | 0.42 | −2.56 |
| Cells_Granularity_13_AGP | 1.61E−02 | 2.29E−02 | 0.25 | −2.27 |
| Cells_Granularity_2_BODIPY | 4.13E−02 | 2.74E−02 | 0.31 | 1.91 |
| Cells_Intensity_IntegratedIntensityEdge_AGP | 3.56E−02 | 2.71E−02 | 0.32 | −1.82 |
| Cells_Intensity_LowerQuartileIntensity_AGP | 2.92E−02 | 2.54E−02 | 0.34 | −2.14 |
| Cells_Intensity_MADIntensity_AGP | 2.29E−02 | 2.31E−02 | 0.36 | −2.43 |
| Cells_Intensity_MADIntensity_Mito | 9.67E−03 | 1.68E−02 | 0.41 | 3.40 |
| Cells_Intensity_MaxIntensity_Mito | 8.35E−03 | 1.51E−02 | 0.38 | 2.93 |
| Cells_Intensity_MaxIntensityEdge_AGP | 2.84E−02 | 2.50E−02 | 0.37 | −2.09 |
| Cells_Intensity_MaxIntensityEdge_Mito | 4.95E−02 | 2.87E−02 | 0.24 | 2.05 |
| Cells_Intensity_MeanIntensity_AGP | 2.65E−02 | 2.44E−02 | 0.36 | −2.17 |
| Cells_Intensity_MeanIntensity_Mito | 4.10E−02 | 2.74E−02 | 0.25 | 2.21 |
| Cells_Intensity_MeanIntensityEdge_AGP | 4.68E−02 | 2.79E−02 | 0.30 | −1.90 |
| Cells_Intensity_MedianIntensity_AGP | 2.06E−02 | 2.29E−02 | 0.38 | −2.34 |
| Cells_Intensity_MinIntensityEdge_AGP | 4.43E−02 | 2.76E−02 | 0.30 | −1.76 |
| Cells_Intensity_StdIntensity_Mito | 6.08E−03 | 1.29E−02 | 0.45 | 3.88 |
| Cells_Intensity_StdIntensityEdge_AGP | 1.77E−02 | 2.29E−02 | 0.40 | −2.57 |
| Cells_Intensity_StdIntensityEdge_Mito | 4.14E−02 | 2.74E−02 | 0.27 | 2.31 |
| Cells_Intensity_UpperQuartileIntensity_AGP | 2.44E−02 | 2.38E−02 | 0.37 | −2.25 |
| Cells_Intensity_UpperQuartileIntensity_Mito | 2.65E−02 | 2.44E−02 | 0.31 | 2.58 |
| Cells_Mean_LargeBODIPYObjects_Correlation_Correlation_Mito_BODIPY | 2.77E−02 | 2.46E−02 | 0.32 | 2.42 |
| Cells_Mean_LargeBODIPYObjects_Correlation_K_AGP_DNA | 4.66E−02 | 2.79E−02 | 0.21 | 2.49 |
| Cells_Mean_LargeBODIPYObjects_Correlation_K_DNA_Mito | 3.47E−02 | 2.71E−02 | 0.28 | 2.53 |
| Cells_Mean_LargeBODIPYObjects_Correlation_K_Mito_AGP | 1.72E−02 | 2.29E−02 | 0.32 | −1.74 |
| Cells_Mean_LargeBODIPYObjects_Correlation_K_Mito_DNA | 1.60E−02 | 2.29E−02 | 0.33 | −1.87 |
| Cytoplasm_Correlation_K_AGP_DNA | 3.43E−02 | 2.71E−02 | 0.31 | 2.93 |
| Cytoplasm_Correlation_K_AGP_Mito | 9.43E−03 | 1.64E−02 | 0.42 | 3.26 |
| Cytoplasm_Correlation_K_BODIPY_AGP | 2.06E−02 | 2.29E−02 | 0.40 | −2.05 |
| Cytoplasm_Correlation_K_DNA_AGP | 1.90E−02 | 2.29E−02 | 0.36 | −2.26 |
| Cytoplasm_Correlation_K_DNA_Mito | 1.00E−02 | 1.72E−02 | 0.22 | 1.57 |
| Cytoplasm_Correlation_K_Mito_AGP | 6.53E−04 | 1.28E−02 | 0.56 | −2.92 |
| Cytoplasm_Correlation_K_Mito_DNA | 2.29E−02 | 2.31E−02 | 0.23 | −1.34 |
| Cytoplasm_Correlation_Overlap_BODIPY_AGP | 4.63E−02 | 2.79E−02 | 0.24 | −1.70 |
| Cytoplasm_Correlation_Overlap_Mito_BODIPY | 4.95E−02 | 2.87E−02 | 0.28 | −1.74 |
| Cytoplasm_Granularity_1_BODIPY | 4.93E−02 | 2.87E−02 | 0.25 | −1.77 |
| Cytoplasm_Granularity_1_Mito | 8.07E−03 | 1.48E−02 | 0.42 | −2.55 |
| Cytoplasm_Granularity_13_AGP | 1.49E−02 | 2.29E−02 | 0.25 | −2.31 |
| Cytoplasm_Granularity_2_BODIPY | 4.42E−02 | 2.76E−02 | 0.29 | 1.93 |
| Cytoplasm_Intensity_IntegratedIntensityEdge_AGP | 3.07E−02 | 2.62E−02 | 0.33 | −1.90 |
| Cytoplasm_Intensity_LowerQuartileIntensity_AGP | 3.22E−02 | 2.68E−02 | 0.33 | −2.05 |
| Cytoplasm_Intensity_MADIntensity_AGP | 2.57E−02 | 2.44E−02 | 0.34 | −2.18 |
| Cytoplasm_Intensity_MADIntensity_Mito | 1.28E−02 | 2.10E−02 | 0.37 | 2.93 |
| Cytoplasm_Intensity_MaxIntensity_Mito | 8.77E−03 | 1.56E−02 | 0.37 | 2.86 |
| Cytoplasm_Intensity_MaxIntensityEdge_AGP | 4.12E−02 | 2.74E−02 | 0.32 | −2.00 |
| Cytoplasm_Intensity_MaxIntensityEdge_Mito | 1.77E−02 | 2.29E−02 | 0.34 | 2.85 |
| Cytoplasm_Intensity_MeanIntensity_AGP | 2.64E−02 | 2.44E−02 | 0.35 | −2.09 |
| Cytoplasm_Intensity_MeanIntensity_Mito | 4.70E−02 | 2.80E−02 | 0.23 | 2.00 |
| Cytoplasm_Intensity_MeanIntensityEdge_AGP | 3.03E−02 | 2.61E−02 | 0.36 | −2.16 |
| Cytoplasm_Intensity_MedianIntensity_AGP | 2.25E−02 | 2.31E−02 | 0.37 | −2.21 |
| Cytoplasm_Intensity_MinIntensityEdge_AGP | 4.40E−02 | 2.76E−02 | 0.30 | −1.77 |
| Cytoplasm_Intensity_StdIntensity_Mito | 6.72E−03 | 1.33E−02 | 0.43 | 3.47 |
| Cytoplasm_Intensity_StdIntensityEdge_AGP | 2.43E−02 | 2.37E−02 | 0.35 | −2.54 |
| Cytoplasm_Intensity_StdIntensityEdge_Mito | 1.51E−02 | 2.29E−02 | 0.38 | 3.27 |
| Cytoplasm_Intensity_UpperQuartileIntensity_AGP | 2.35E−02 | 2.32E−02 | 0.36 | −2.15 |
| Cytoplasm_Intensity_UpperQuartileIntensity_Mito | 3.20E−02 | 2.68E−02 | 0.28 | 2.28 |
| Nuclei_AreaShape_MaximumRadius | 4.38E−02 | 2.76E−02 | 0.25 | −2.04 |
| Nuclei_AreaShape_MeanRadius | 4.92E−02 | 2.87E−02 | 0.25 | −2.00 |
| Nuclei_AreaShape_MinFeretDiameter | 4.43E−02 | 2.76E−02 | 0.24 | −1.93 |
| Nuclei_AreaShape_MinorAxisLength | 4.41E−02 | 2.76E−02 | 0.25 | −1.97 |
| Nuclei_Correlation_K_AGP_DNA | 2.83E−02 | 2.50E−02 | 0.32 | 2.84 |
| Nuclei_Correlation_K_AGP_Mito | 1.19E−02 | 1.98E−02 | 0.42 | 3.46 |
| Nuclei_Correlation_K_BODIPY_AGP | 4.40E−02 | 2.76E−02 | 0.24 | −1.77 |
| Nuclei_Correlation_K_DNA_AGP | 1.61E−02 | 2.29E−02 | 0.38 | −2.50 |
| Nuclei_Correlation_K_Mito_AGP | 5.80E−04 | 1.28E−02 | 0.56 | −2.96 |
| Nuclei_Correlation_K_Mito_DNA | 2.28E−02 | 2.31E−02 | 0.28 | −1.57 |
| Nuclei_Correlation_Overlap_DNA_Mito | 3.49E−03 | 1.28E−02 | 0.51 | −3.45 |
| Nuclei_Granularity_1_Mito | 1.52E−02 | 2.29E−02 | 0.38 | −2.31 |
| Nuclei_Granularity_13_AGP | 3.81E−02 | 2.72E−02 | 0.19 | −2.08 |
| Nuclei_Granularity_2_DNA | 4.88E−02 | 2.85E−02 | 0.25 | 2.07 |
| Nuclei_Granularity_7_DNA | 2.34E−02 | 2.32E−02 | 0.17 | −1.48 |
| Nuclei_Intensity_IntegratedIntensity_AGP | 3.68E−02 | 2.71E−02 | 0.31 | −1.95 |
| Nuclei_Intensity_IntegratedIntensity_Mito | 4.24E−02 | 2.76E−02 | 0.26 | 2.00 |
| Nuclei_Intensity_IntegratedIntensityEdge_AGP | 3.04E−02 | 2.61E−02 | 0.33 | −2.03 |
| Nuclei_Intensity_IntegratedIntensityEdge_Mito | 1.98E−02 | 2.29E−02 | 0.34 | 2.74 |
| Nuclei_Intensity_LowerQuartileIntensity_AGP | 8.98E−03 | 1.59E−02 | 0.45 | −3.00 |
| Nuclei_Intensity_LowerQuartileIntensity_Mito | 2.11E−02 | 2.30E−02 | 0.35 | 2.97 |
| Nuclei_Intensity_MADIntensity_DNA | 4.55E−02 | 2.77E−02 | 0.23 | 2.32 |
| Nuclei_Intensity_MADIntensity_Mito | 8.04E−03 | 1.48E−02 | 0.47 | 4.66 |
| Nuclei_Intensity_MaxIntensity_Mito | 1.07E−02 | 1.80E−02 | 0.40 | 3.35 |
| Nuclei_Intensity_MaxIntensityEdge_AGP | 4.30E−02 | 2.76E−02 | 0.32 | −2.00 |
| Nuclei_Intensity_MaxIntensityEdge_Mito | 1.07E−02 | 1.80E−02 | 0.40 | 3.31 |
| Nuclei_Intensity_MeanIntensity_AGP | 1.93E−02 | 2.29E−02 | 0.41 | −2.57 |
| Nuclei_Intensity_MeanIntensity_Mito | 1.58E−02 | 2.29E−02 | 0.38 | 3.26 |
| Nuclei_Intensity_MeanIntensityEdge_AGP | 1.65E−02 | 2.29E−02 | 0.40 | −2.50 |
| Nuclei_Intensity_MeanIntensityEdge_Mito | 1.36E−02 | 2.21E−02 | 0.39 | 3.15 |
| Nuclei_Intensity_MedianIntensity_AGP | 1.54E−02 | 2.29E−02 | 0.42 | −2.73 |
| Nuclei_Intensity_MedianIntensity_Mito | 1.74E−02 | 2.29E−02 | 0.37 | 3.22 |
| Nuclei_Intensity_MinIntensity_AGP | 1.06E−02 | 1.80E−02 | 0.44 | −2.82 |
| Nuclei_Intensity_MinIntensity_Mito | 3.63E−02 | 2.71E−02 | 0.30 | 2.35 |
| Nuclei_Intensity_MinIntensityEdge_AGP | 1.06E−02 | 1.80E−02 | 0.44 | −2.80 |
| Nuclei_Intensity_MinIntensityEdge_Mito | 3.01E−02 | 2.60E−02 | 0.31 | 2.43 |
| Nuclei_Intensity_StdIntensity_Mito | 8.71E−03 | 1.55E−02 | 0.45 | 4.36 |
| Nuclei_Intensity_StdIntensityEdge_Mito | 9.90E−03 | 1.71E−02 | 0.42 | 3.83 |
| Nuclei_Intensity_UpperQuartileIntensity_AGP | 2.74E−02 | 2.46E−02 | 0.38 | −2.38 |
| Nuclei_Intensity_UpperQuartileIntensity_Mito | 1.43E−02 | 2.29E−02 | 0.39 | 3.45 |
| Cells_Texture_AngularSecondMoment_AGP_20_01 | 2.04E−02 | 2.29E−02 | 0.32 | 3.18 |
| Cells_Texture_AngularSecondMoment_Mito_20_02 | 4.39E−03 | 1.28E−02 | 0.39 | −2.00 |
| Cells_Texture_Contrast_Mito_20_03 | 1.94E−02 | 2.29E−02 | 0.37 | 3.85 |
| Cells_Texture_Correlation_DNA_5_00 | 3.54E−02 | 2.71E−02 | 0.27 | −2.36 |
| Cells_Texture_Correlation_BODIPY_20_02 | 2.13E−03 | 1.28E−02 | 0.58 | −3.99 |
| Cells_Texture_DifferenceEntropy_AGP_20_03 | 4.15E−02 | 2.74E−02 | 0.32 | −2.09 |
| Cells_Texture_DifferenceEntropy_Mito_20_03 | 2.21E−03 | 1.28E−02 | 0.52 | 3.50 |
| Cells_Texture_DifferenceVariance_AGP_20_03 | 4.52E−02 | 2.77E−02 | 0.28 | 2.55 |
| Cells_Texture_DifferenceVariance_Mito_20_03 | 4.49E−03 | 1.28E−02 | 0.39 | −2.00 |
| Cells_Texture_Entropy_AGP_20_01 | 2.53E−02 | 2.43E−02 | 0.34 | −2.47 |
| Cells_Texture_Entropy_Mito_20_02 | 3.78E−03 | 1.28E−02 | 0.46 | 2.93 |
| Cells_Texture_InfoMeas1_DNA_5_03 | 1.15E−02 | 1.92E−02 | 0.30 | 2.47 |
| Cells_Texture_InfoMeas1_BODIPY_10_00 | 2.54E−02 | 2.43E−02 | 0.37 | 2.25 |
| Cells_Texture_InfoMeas2_Mito_5_02 | 5.66E−03 | 1.28E−02 | 0.39 | 3.38 |
| Cells_Texture_InverseDifferenceMoment_AGP_20_01 | 1.94E−02 | 2.29E−02 | 0.38 | 2.82 |
| Cells_Texture_InverseDifferenceMoment_Mito_20_03 | 1.63E−03 | 1.28E−02 | 0.51 | −2.93 |
| Cells_Texture_SumAverage_AGP_20_03 | 2.28E−02 | 2.31E−02 | 0.38 | −2.25 |
| Cells_Texture_SumAverage_Mito_20_01 | 3.15E−02 | 2.66E−02 | 0.28 | 2.41 |
| Cells_Texture_SumEntropy_AGP_5_00 | 3.49E−02 | 2.71E−02 | 0.31 | −2.37 |
| Cells_Texture_SumEntropy_Mito_20_01 | 3.81E−03 | 1.28E−02 | 0.47 | 3.07 |
| Cells_Texture_SumVariance_Mito_10_01 | 1.96E−02 | 2.29E−02 | 0.37 | 3.94 |
| Cells_Texture_Variance_Mito_20_03 | 1.72E−02 | 2.29E−02 | 0.38 | 3.94 |
| Nuclei_AreaShape_Zernike_8_8 | 4.75E−02 | 2.81E−02 | 0.30 | 2.82 |
| Nuclei_RadialDistribution_FracAtD_Mito_2of4 | 2.72E−02 | 2.44E−02 | 0.34 | −2.45 |
| Nuclei_Texture_AngularSecondMoment_AGP_20_02 | 4.52E−02 | 2.77E−02 | 0.30 | 2.39 |
| Nuclei_Texture_AngularSecondMoment_DNA_10_03 | 2.72E−02 | 2.44E−02 | 0.29 | −2.08 |
| Nuclei_Texture_AngularSecondMoment_BODIPY_5_00 | 3.75E−02 | 2.71E−02 | 0.29 | −2.80 |
| Nuclei_Texture_AngularSecondMoment_Mito_5_02 | 7.48E−04 | 1.28E−02 | 0.55 | −2.85 |
| Nuclei_Texture_Contrast_Mito_5_00 | 2.46E−02 | 2.38E−02 | 0.35 | 3.85 |
| Nuclei_Texture_Correlation_BODIPY_20_02 | 1.24E−02 | 2.05E−02 | 0.42 | 2.39 |
| Nuclei_Texture_Correlation_Mito_5_02 | 1.22E−02 | 2.01E−02 | 0.45 | 4.46 |
| Nuclei_Texture_DifferenceEntropy_AGP_20_02 | 4.78E−02 | 2.82E−02 | 0.32 | −1.95 |
| Nuclei_Texture_DifferenceEntropy_Mito_10_03 | 1.81E−03 | 1.28E−02 | 0.55 | 4.15 |
| Nuclei_Texture_DifferenceVariance_DNA_10_03 | 3.53E−02 | 2.71E−02 | 0.23 | −1.90 |
| Nuclei_Texture_DifferenceVariance_Mito_10_03 | 2.14E−03 | 1.28E−02 | 0.46 | −2.33 |
| Nuclei_Texture_Entropy_AGP_20_00 | 4.44E−02 | 2.76E−02 | 0.32 | −2.05 |
| Nuclei_Texture_Entropy_DNA_10_00 | 4.93E−02 | 2.87E−02 | 0.22 | 1.94 |
| Nuclei_Texture_Entropy_Mito_10_02 | 1.52E−03 | 1.28E−02 | 0.57 | 4.21 |
| Nuclei_Texture_InfoMeas2_Mito_5_00 | 3.55E−03 | 1.28E−02 | 0.53 | 4.75 |
| Nuclei_Texture_InverseDifferenceMoment_AGP_20_02 | 4.43E−02 | 2.76E−02 | 0.32 | 2.14 |
| Nuclei_Texture_InverseDifferenceMoment_DNA_10_00 | 3.78E−02 | 2.72E−02 | 0.26 | −2.16 |
| Nuclei_Texture_InverseDifferenceMoment_Mito_10_03 | 9.70E−04 | 1.28E−02 | 0.60 | −4.04 |
| Nuclei_Texture_SumAverage_AGP_10_03 | 1.75E−02 | 2.29E−02 | 0.41 | −2.59 |
| Nuclei_Texture_SumAverage_Mito_10_03 | 1.63E−02 | 2.29E−02 | 0.37 | 3.27 |
| Nuclei_Texture_SumEntropy_Mito_5_00 | 1.87E−03 | 1.28E−02 | 0.56 | 4.31 |
| Nuclei_Texture_SumVariance_Mito_10_03 | 3.15E−02 | 2.66E−02 | 0.34 | 3.89 |
| Nuclei_Texture_Variance_Mito_10_00 | 3.34E−02 | 2.71E−02 | 0.33 | 3.82 |
| Cytoplasm_RadialDistribution_FracAtD_Mito_2of4 | 4.60E−02 | 2.78E−02 | 0.18 | 1.31 |
| Cytoplasm_Texture_AngularSecondMoment_AGP_5_02 | 1.86E−02 | 2.29E−02 | 0.31 | 2.97 |
| Cytoplasm_Texture_AngularSecondMoment_Mito_20_02 | 6.12E−03 | 1.29E−02 | 0.36 | −1.88 |
| Cytoplasm_Texture_Contrast_Mito_20_03 | 1.77E−02 | 2.29E−02 | 0.36 | 3.56 |
| Cytoplasm_Texture_DifferenceEntropy_AGP_20_01 | 4.19E−02 | 2.76E−02 | 0.29 | −2.03 |
| Cytoplasm_Texture_DifferenceEntropy_Mito_20_03 | 2.42E−03 | 1.28E−02 | 0.49 | 3.15 |
| Cytoplasm_Texture_DifferenceVariance_AGP_20_02 | 3.49E−02 | 2.71E−02 | 0.27 | 2.54 |
| Cytoplasm_Texture_DifferenceVariance_BODIPY_5_00 | 4.36E−02 | 2.76E−02 | 0.31 | −1.79 |
| Cytoplasm_Texture_DifferenceVariance_Mito_20_03 | 5.51E−03 | 1.28E−02 | 0.37 | −1.91 |
| Cytoplasm_Texture_Entropy_AGP_5_02 | 2.91E−02 | 2.54E−02 | 0.30 | −2.19 |
| Cytoplasm_Texture_Entropy_Mito_20_00 | 5.27E−03 | 1.28E−02 | 0.42 | 2.59 |
| Cytoplasm_Texture_InfoMeas1_AGP_5_02 | 3.05E−02 | 2.61E−02 | 0.22 | −1.87 |
| Cytoplasm_Texture_InfoMeas2_Mito_5_00 | 1.79E−03 | 1.28E−02 | 0.47 | 3.50 |
| Cytoplasm_Texture_InverseDifferenceMoment_AGP_20_01 | 1.85E−02 | 2.29E−02 | 0.35 | 2.61 |
| Cytoplasm_Texture_InverseDifferenceMoment_DNA_10_00 | 4.16E−02 | 2.74E−02 | 0.23 | −2.81 |
| Cytoplasm_Texture_InverseDifferenceMoment_Mito_20_03 | 1.99E−03 | 1.28E−02 | 0.47 | −2.69 |
| Cytoplasm_Texture_SumAverage_AGP_20_01 | 2.56E−02 | 2.44E−02 | 0.36 | −2.11 |
| Cytoplasm_Texture_SumAverage_DNA_10_02 | 3.76E−02 | 2.71E−02 | 0.24 | 3.07 |
| Cytoplasm_Texture_SumAverage_Mito_20_01 | 4.00E−02 | 2.72E−02 | 0.25 | 2.09 |
| Cytoplasm_Texture_SumEntropy_AGP_5_00 | 3.56E−02 | 2.71E−02 | 0.28 | −2.16 |
| Cytoplasm_Texture_SumEntropy_Mito_10_01 | 5.01E−03 | 1.28E−02 | 0.43 | 2.71 |
| Cytoplasm_Texture_SumVariance_Mito_10_01 | 0.019858421 | 0.022916244 | 0.36 | 3.52 |
| Cytoplasm_Texture_Variance_Mito_20_03 | 0.019088676 | 0.022916244 | 0.36 | 3.53 |

TABLE 17
rs12454712-mediated LipocyteProfiler in subcutaneous AMSCs at day14. (ANOVA
adj. BMI, sex, age, batch, significance level 5% FDR). P-value, p-value of ANOVA, q-value,
q-value of ANOVA, FDR; eta_sq, eta square of ANOVA, effect size; F value of ANOVA;
t-statistics of t-test.

| Lipocyte Profiler features | p-value | q-value | eta_sq | t-statistics |
|---|---|---|---|---|
| Cells_AreaShape_Orientation | 4.75E−03 | 3.63E−02 | 0.42 | −2.31 |
| Cells_Correlation_K_AGP_BODIPY | 4.15E−02 | 3.63E−02 | 0.22 | 2.26 |
| Cells_Correlation_K_DNA_AGP | 1.24E−02 | 3.63E−02 | 0.28 | −2.57 |
| Cells_Correlation_K_Mito_AGP | 1.21E−02 | 3.63E−02 | 0.31 | −1.86 |
| Cells_Correlation_Overlap_BODIPY_AGP | 4.13E−02 | 3.63E−02 | 0.24 | −1.93 |
| Cells_Correlation_Overlap_Mito_BODIPY | 2.08E−02 | 3.63E−02 | 0.29 | −2.09 |
| Cells_Intensity_IntegratedIntensity_AGP | 4.67E−02 | 3.70E−02 | 0.20 | −1.85 |
| Cells_Intensity_IntegratedIntensityEdge_AGP | 1.68E−02 | 3.63E−02 | 0.30 | −2.03 |
| Cells_Intensity_LowerQuartileIntensity_AGP | 1.59E−02 | 3.63E−02 | 0.26 | −1.92 |
| Cells_Intensity_MADIntensity_AGP | 2.39E−02 | 3.63E−02 | 0.26 | −2.37 |
| Cells_Intensity_MaxIntensity_AGP | 1.96E−02 | 3.63E−02 | 0.26 | −2.73 |
| Cells_Intensity_MaxIntensityEdge_AGP | 1.41E−02 | 3.63E−02 | 0.30 | −2.14 |
| Cells_Intensity_MeanIntensity_AGP | 1.44E−02 | 3.63E−02 | 0.29 | −2.11 |
| Cells_Intensity_MeanIntensityEdge_AGP | 1.51E−02 | 3.63E−02 | 0.27 | −1.95 |
| Cells_Intensity_MedianIntensity_AGP | 1.65E−02 | 3.63E−02 | 0.27 | −2.01 |
| Cells_Intensity_MinIntensity_AGP | 7.91E−03 | 3.63E−02 | 0.36 | −1.90 |
| Cells_Intensity_MinIntensityEdge_AGP | 1.03E−02 | 3.63E−02 | 0.34 | −1.90 |
| Cells_Intensity_StdIntensity_AGP | 2.74E−02 | 3.63E−02 | 0.26 | −2.61 |
| Cells_Intensity_StdIntensityEdge_AGP | 2.25E−02 | 3.63E−02 | 0.27 | −2.15 |
| Cells_Intensity_UpperQuartileIntensity_AGP | 1.53E−02 | 3.63E−02 | 0.29 | −2.14 |
| Cells_Mean_LargeBODIPYObjects_AreaShape_Area | 2.79E−02 | 3.63E−02 | 0.24 | 2.42 |
| Cells_Mean_LargeBODIPYObjects_AreaShape_Compactness | 3.49E−02 | 3.63E−02 | 0.22 | 2.25 |
| Cells_Mean_LargeBODIPYObjects_AreaShape_Extent | 3.25E−02 | 3.63E−02 | 0.23 | 2.23 |
| Cells_Mean_LargeBODIPYObjects_AreaShape_FormFactor | 4.56E−02 | 3.65E−02 | 0.23 | 1.72 |
| Cells_Mean_LargeBODIPYObjects_AreaShape_MajorAxisLength | 3.35E−02 | 3.63E−02 | 0.23 | 2.29 |
| Cells_Mean_LargeBODIPYObjects_AreaShape_MaxFeretDiameter | 3.35E−02 | 3.63E−02 | 0.23 | 2.29 |
| Cells_Mean_LargeBODIPYObjects_AreaShape_MaximumRadius | 2.60E−02 | 3.63E−02 | 0.24 | 2.35 |
| Cells_Mean_LargeBODIPYObjects_AreaShape_MeanRadius | 2.48E−02 | 3.63E−02 | 0.24 | 2.41 |
| Cells_Mean_LargeBODIPYObjects_AreaShape_MedianRadius | 2.47E−02 | 3.63E−02 | 0.24 | 2.42 |
| Cells_Mean_LargeBODIPYObjects_AreaShape_MinFeretDiameter | 3.10E−02 | 3.63E−02 | 0.23 | 2.33 |
| Cells_Mean_LargeBODIPYObjects_AreaShape_MinorAxisLength | 3.00E−02 | 3.63E−02 | 0.23 | 2.33 |
| Cells_Mean_LargeBODIPYObjects_AreaShape_Perimeter | 3.43E−02 | 3.63E−02 | 0.22 | 2.28 |
| Cells_Mean_LargeBODIPYObjects_AreaShape_Solidity | 1.58E−02 | 3.63E−02 | 0.32 | 2.23 |
| Cells_Mean_LargeBODIPYObjects_Correlation_K_Mito_AGP | 8.48E−03 | 3.63E−02 | 0.35 | −2.12 |
| Cells_Mean_LargeBODIPYObjects_Correlation_K_Mito_DNA | 1.18E−02 | 3.63E−02 | 0.27 | −1.93 |
| Cells_Neighbors_NumberOfNeighbors_Adjacent | 3.64E−02 | 3.63E−02 | 0.20 | −2.31 |
| Cytoplasm_AreaShape_Compactness | 1.91E−02 | 3.63E−02 | 0.26 | −1.71 |
| Cytoplasm_AreaShape_Extent | 1.26E−02 | 3.63E−02 | 0.27 | 2.13 |
| Cytoplasm_AreaShape_FormFactor | 9.93E−03 | 3.63E−02 | 0.23 | 2.20 |
| Cytoplasm_AreaShape_Orientation | 4.40E−03 | 3.63E−02 | 0.43 | −2.34 |
| Cytoplasm_AreaShape_Solidity | 6.32E−03 | 3.63E−02 | 0.32 | 2.64 |
| Cytoplasm_Correlation_K_AGP_BODIPY | 4.90E−02 | 3.75E−02 | 0.22 | 2.31 |
| Cytoplasm_Correlation_K_DNA_AGP | 1.47E−02 | 3.63E−02 | 0.24 | −2.87 |
| Cytoplasm_Correlation_K_Mito_AGP | 1.20E−02 | 3.63E−02 | 0.31 | −1.85 |
| Cytoplasm_Correlation_Overlap_BODIPY_AGP | 2.12E−02 | 3.63E−02 | 0.30 | −1.83 |
| Cytoplasm_Correlation_Overlap_DNA_BODIPY | 3.66E−02 | 3.63E−02 | 0.23 | −2.27 |
| Cytoplasm_Correlation_Overlap_Mito_BODIPY | 3.39E−02 | 3.63E−02 | 0.27 | −1.83 |
| Cytoplasm_Granularity_4_BODIPY | 3.95E−02 | 3.63E−02 | 0.23 | 1.97 |
| Cytoplasm_Granularity_4_Mito | 4.73E−02 | 3.71E−02 | 0.19 | 2.67 |
| Cytoplasm_Intensity_IntegratedIntensityEdge_AGP | 1.36E−02 | 3.63E−02 | 0.31 | −2.08 |
| Cytoplasm_Intensity_LowerQuartileIntensity_AGP | 1.70E−02 | 3.63E−02 | 0.26 | −1.91 |
| Cytoplasm_Intensity_MADIntensity_AGP | 2.82E−02 | 3.63E−02 | 0.25 | −2.20 |
| Cytoplasm_Intensity_MaxIntensity_AGP | 2.76E−02 | 3.63E−02 | 0.22 | −2.51 |
| Cytoplasm_Intensity_MaxIntensityEdge_AGP | 2.10E−02 | 3.63E−02 | 0.29 | −2.31 |
| Cytoplasm_Intensity_MeanIntensity_AGP | 1.64E−02 | 3.63E−02 | 0.28 | −2.08 |
| Cytoplasm_Intensity_MeanIntensityEdge_AGP | 1.31E−02 | 3.63E−02 | 0.29 | −1.99 |
| Cytoplasm_Intensity_MedianIntensity_AGP | 1.86E−02 | 3.63E−02 | 0.26 | −1.97 |
| Cytoplasm_Intensity_MinIntensity_AGP | 7.95E−03 | 3.63E−02 | 0.36 | −1.90 |
| Cytoplasm_Intensity_MinIntensityEdge_AGP | 1.01E−02 | 3.63E−02 | 0.34 | −1.90 |
| Cytoplasm_Intensity_StdIntensity_AGP | 3.34E−02 | 3.63E−02 | 0.24 | −2.49 |
| Cytoplasm_Intensity_StdIntensityEdge_AGP | 3.78E−02 | 3.63E−02 | 0.25 | −2.20 |
| Cytoplasm_Intensity_UpperQuartileIntensity_AGP | 1.80E−02 | 3.63E−02 | 0.28 | −2.07 |
| Nuclei_AreaShape_Area | 1.70E−02 | 3.63E−02 | 0.25 | −2.11 |
| Nuclei_AreaShape_MajorAxisLength | 2.89E−02 | 3.63E−02 | 0.21 | −1.93 |
| Nuclei_AreaShape_MaxFeretDiameter | 2.87E−02 | 3.63E−02 | 0.21 | −1.91 |
| Nuclei_AreaShape_MaximumRadius | 2.42E−02 | 3.63E−02 | 0.23 | −2.23 |
| Nuclei_AreaShape_MeanRadius | 3.04E−02 | 3.63E−02 | 0.23 | −2.25 |
| Nuclei_AreaShape_MedianRadius | 2.86E−02 | 3.63E−02 | 0.22 | −2.36 |
| Nuclei_AreaShape_MinFeretDiameter | 2.04E−02 | 3.63E−02 | 0.23 | −2.11 |
| Nuclei_AreaShape_MinorAxisLength | 2.27E−02 | 3.63E−02 | 0.22 | −2.12 |
| Nuclei_AreaShape_Orientation | 3.13E−03 | 3.63E−02 | 0.48 | −3.07 |
| Nuclei_AreaShape_Perimeter | 2.24E−02 | 3.63E−02 | 0.21 | −1.98 |
| Nuclei_Correlation_Correlation_DNA_AGP | 4.72E−02 | 3.71E−02 | 0.22 | 1.56 |
| Nuclei_Correlation_K_BODIPY_AGP | 1.80E−02 | 3.63E−02 | 0.29 | −1.71 |
| Nuclei_Correlation_K_DNA_AGP | 1.27E−02 | 3.63E−02 | 0.29 | −2.58 |
| Nuclei_Correlation_K_Mito_AGP | 9.76E−03 | 3.63E−02 | 0.34 | −1.97 |
| Nuclei_Granularity_1_Mito | 2.14E−02 | 3.63E−02 | 0.26 | −1.51 |
| Nuclei_Granularity_7_DNA | 1.42E−02 | 3.63E−02 | 0.23 | −1.62 |
| Nuclei_Granularity_8_DNA | 4.61E−02 | 3.68E−02 | 0.22 | −1.32 |
| Nuclei_Intensity_IntegratedIntensity_AGP | 1.58E−02 | 3.63E−02 | 0.28 | −2.00 |
| Nuclei_Intensity_IntegratedIntensityEdge_AGP | 1.69E−02 | 3.63E−02 | 0.28 | −2.06 |
| Nuclei_Intensity_LowerQuartileIntensity_AGP | 2.04E−02 | 3.63E−02 | 0.28 | −2.28 |
| Nuclei_Intensity_MaxIntensity_AGP | 2.36E−02 | 3.63E−02 | 0.29 | −2.32 |
| Nuclei_Intensity_MaxIntensityEdge_AGP | 2.13E−02 | 3.63E−02 | 0.29 | −2.32 |
| Nuclei_Intensity_MeanIntensity_AGP | 2.48E−02 | 3.63E−02 | 0.28 | −2.19 |
| Nuclei_Intensity_MeanIntensityEdge_AGP | 1.92E−02 | 3.63E−02 | 0.30 | −2.17 |
| Nuclei_Intensity_MedianIntensity_AGP | 2.46E−02 | 3.63E−02 | 0.28 | −2.21 |
| Nuclei_Intensity_MinIntensity_AGP | 1.41E−02 | 3.63E−02 | 0.31 | −2.22 |
| Nuclei_Intensity_MinIntensityEdge_AGP | 1.37E−02 | 3.63E−02 | 0.32 | −2.19 |
| Nuclei_Intensity_StdIntensity_AGP | 4.34E−02 | 3.63E−02 | 0.24 | −1.99 |
| Nuclei_Intensity_StdIntensityEdge_AGP | 2.96E−02 | 3.63E−02 | 0.27 | −2.29 |
| Nuclei_Intensity_UpperQuartileIntensity_AGP | 2.95E−02 | 3.63E−02 | 0.27 | −2.13 |
| Cells_AreaShape_Zernike_2_2 | 3.35E−02 | 3.63E−02 | 0.20 | 2.54 |
| Cells_RadialDistribution_FracAtD_DNA_1of4 | 2.29E−02 | 3.63E−02 | 0.26 | −1.81 |
| Cells_RadialDistribution_MeanFrac_DNA_1of4 | 1.56E−02 | 3.63E−02 | 0.29 | −1.98 |
| Cells_RadialDistribution_RadialCV_AGP_4of4 | 3.35E−02 | 3.63E−02 | 0.20 | 1.91 |
| Cells_RadialDistribution_RadialCV_DNA_1of4 | 1.18E−02 | 3.63E−02 | 0.35 | 2.52 |
| Cells_RadialDistribution_RadialCV_Mito_3of4 | 1.76E−02 | 3.63E−02 | 0.28 | 2.64 |
| Cells_Texture_AngularSecondMoment_Mito_20_01 | 1.27E−02 | 3.63E−02 | 0.32 | −1.97 |
| Cells_Texture_Contrast_AGP_5_00 | 1.38E−02 | 3.63E−02 | 0.30 | −2.29 |
| Cells_Texture_Correlation_AGP_5_03 | 2.19E−02 | 3.63E−02 | 0.21 | 1.71 |
| Cells_Texture_Correlation_DNA_20_01 | 4.96E−02 | 3.77E−02 | 0.18 | −1.96 |
| Cells_Texture_Correlation_BODIPY_5_00 | 2.13E−02 | 3.63E−02 | 0.29 | 2.62 |
| Cells_Texture_Correlation_Mito_5_00 | 2.68E−02 | 3.63E−02 | 0.27 | 3.23 |
| Cells_Texture_DifferenceEntropy_AGP_20_03 | 3.80E−02 | 3.63E−02 | 0.25 | −2.60 |
| Cells_Texture_DifferenceEntropy_Mito_20_01 | 3.39E−02 | 3.63E−02 | 0.21 | 1.57 |
| Cells_Texture_DifferenceVariance_Mito_20_01 | 1.73E−02 | 3.63E−02 | 0.28 | −1.84 |
| Cells_Texture_Entropy_AGP_10_01 | 3.55E−02 | 3.63E−02 | 0.24 | −2.59 |
| Cells_Texture_Entropy_Mito_20_01 | 2.41E−02 | 3.63E−02 | 0.23 | 1.66 |
| Cells_Texture_InfoMeas1_AGP_5_00 | 4.35E−02 | 3.63E−02 | 0.21 | −2.30 |
| Cells_Texture_InfoMeas2_BODIPY_5_00 | 3.94E−02 | 3.63E−02 | 0.23 | 1.85 |
| Cells_Texture_InfoMeas2_Mito_5_00 | 1.64E−02 | 3.63E−02 | 0.30 | 2.18 |
| Cells_Texture_InverseDifferenceMoment_AGP_10_03 | 4.41E−02 | 3.63E−02 | 0.22 | 2.56 |
| Cells_Texture_InverseDifferenceMoment_Mito_20_01 | 2.19E−02 | 3.63E−02 | 0.26 | −1.84 |
| Cells_Texture_SumAverage_AGP_10_01 | 1.39E−02 | 3.63E−02 | 0.29 | −2.13 |
| Cells_Texture_SumEntropy_AGP_20_02 | 3.70E−02 | 3.63E−02 | 0.24 | −2.75 |
| Cells_Texture_SumEntropy_Mito_20_03 | 2.57E−02 | 3.63E−02 | 0.23 | 1.67 |
| Cells_Texture_SumVariance_AGP_10_02 | 2.26E−02 | 3.63E−02 | 0.28 | −2.51 |
| Cells_Texture_Variance_AGP_5_03 | 2.05E−02 | 3.63E−02 | 0.28 | −2.41 |
| Nuclei_AreaShape_Zernike_8_2 | 3.40E−02 | 3.63E−02 | 0.26 | −2.16 |
| Nuclei_RadialDistribution_FracAtD_DNA_4of4 | 3.28E−02 | 3.63E−02 | 0.21 | −2.41 |
| Nuclei_RadialDistribution_MeanFrac_DNA_2of4 | 3.84E−02 | 3.63E−02 | 0.25 | 3.26 |
| Nuclei_Texture_AngularSecondMoment_DNA_10_01 | 2.11E−02 | 3.63E−02 | 0.23 | −2.13 |
| Nuclei_Texture_AngularSecondMoment_BODIPY_5_00 | 1.53E−02 | 3.63E−02 | 0.27 | −1.75 |
| Nuclei_Texture_AngularSecondMoment_Mito_10_01 | 1.06E−02 | 3.63E−02 | 0.33 | −2.02 |
| Nuclei_Texture_Contrast_AGP_5_00 | 3.35E−02 | 3.63E−02 | 0.27 | −1.88 |
| Nuclei_Texture_Correlation_AGP_10_01 | 2.95E−02 | 3.63E−02 | 0.20 | −2.38 |
| Nuclei_Texture_Correlation_DNA_10_01 | 3.82E−02 | 3.63E−02 | 0.22 | −2.63 |
| Nuclei_Texture_Correlation_Mito_5_03 | 4.08E−02 | 3.63E−02 | 0.27 | 3.18 |
| Nuclei_Texture_DifferenceEntropy_DNA_10_01 | 4.12E−02 | 3.63E−02 | 0.19 | 2.51 |
| Nuclei_Texture_DifferenceEntropy_Mito_10_01 | 4.32E−02 | 3.63E−02 | 0.20 | 1.60 |
| Nuclei_Texture_DifferenceVariance_DNA_20_00 | 4.86E−03 | 3.63E−02 | 0.27 | −2.32 |
| Nuclei_Texture_DifferenceVariance_BODIPY_10_01 | 3.05E−02 | 3.63E−02 | 0.25 | −1.52 |
| Nuclei_Texture_DifferenceVariance_Mito_20_00 | 7.65E−03 | 3.63E−02 | 0.34 | −2.00 |
| Nuclei_Texture_Entropy_AGP_10_03 | 4.24E−02 | 3.63E−02 | 0.24 | −2.25 |
| Nuclei_Texture_Entropy_DNA_10_00 | 3.60E−02 | 3.63E−02 | 0.20 | 2.34 |
| Nuclei_Texture_Entropy_Mito_20_00 | 3.30E−02 | 3.63E−02 | 0.26 | 1.87 |
| Nuclei_Texture_InfoMeas1_DNA_5_00 | 4.05E−02 | 3.63E−02 | 0.23 | −3.48 |
| Nuclei_Texture_InfoMeas2_Mito_20_01 | 1.69E−02 | 3.63E−02 | 0.32 | 2.17 |
| Nuclei_Texture_InverseDifferenceMoment_DNA_10_01 | 1.41E−02 | 3.63E−02 | 0.25 | −2.41 |
| Nuclei_Texture_InverseDifferenceMoment_BODIPY_20_01 | 2.82E−02 | 3.63E−02 | 0.23 | −1.94 |
| Nuclei_Texture_InverseDifferenceMoment_Mito_20_01 | 1.78E−02 | 3.63E−02 | 0.30 | −2.09 |
| Nuclei_Texture_SumAverage_AGP_20_02 | 2.41E−02 | 3.63E−02 | 0.29 | −2.30 |
| Nuclei_Texture_SumEntropy_AGP_10_01 | 4.25E−02 | 3.63E−02 | 0.24 | −2.31 |
| Nuclei_Texture_SumEntropy_DNA_10_00 | 4.67E−02 | 3.70E−02 | 0.19 | 2.60 |
| Nuclei_Texture_SumEntropy_Mito_20_00 | 4.31E−02 | 3.63E−02 | 0.24 | 1.83 |
| Nuclei_Texture_SumVariance_AGP_10_03 | 2.76E−02 | 3.63E−02 | 0.28 | −1.83 |
| Nuclei_Texture_Variance_AGP_20_03 | 3.14E−02 | 3.63E−02 | 0.27 | −1.82 |
| Cytoplasm_AreaShape_Zernike_2_2 | 1.92E−02 | 3.63E−02 | 0.24 | 2.58 |
| Cytoplasm_RadialDistribution_FracAtD_AGP_1of4 | 2.17E−02 | 3.63E−02 | 0.28 | 3.27 |
| Cytoplasm_RadialDistribution_FracAtD_Mito_3of4 | 1.31E−02 | 3.63E−02 | 0.29 | 2.00 |
| Cytoplasm_RadialDistribution_RadialCV_AGP_4of4 | 4.49E−02 | 3.63E−02 | 0.17 | 1.88 |
| Cytoplasm_RadialDistribution_RadialCV_Mito_3of4 | 2.27E−02 | 3.63E−02 | 0.22 | 1.87 |
| Cytoplasm_Texture_AngularSecondMoment_Mito_20_01 | 1.23E−02 | 3.63E−02 | 0.31 | −2.01 |
| Cytoplasm_Texture_Contrast_AGP_10_03 | 1.88E−02 | 3.63E−02 | 0.28 | −2.23 |
| Cytoplasm_Texture_Correlation_AGP_5_00 | 1.25E−02 | 3.63E−02 | 0.21 | 2.03 |
| Cytoplasm_Texture_Correlation_BODIPY_5_03 | 2.30E−02 | 3.63E−02 | 0.29 | 2.50 |
| Cytoplasm_Texture_Correlation_Mito_5_00 | 2.78E−02 | 3.63E−02 | 0.26 | 2.93 |
| Cytoplasm_Texture_DifferenceEntropy_AGP_10_03 | 3.21E−02 | 3.63E−02 | 0.25 | −2.58 |
| Cytoplasm_Texture_DifferenceEntropy_Mito_20_01 | 3.78E−02 | 3.63E−02 | 0.20 | 1.51 |
| Cytoplasm_Texture_DifferenceVariance_Mito_20_01 | 1.89E−02 | 3.63E−02 | 0.28 | −1.83 |
| Cytoplasm_Texture_Entropy_AGP_5_03 | 3.77E−02 | 3.63E−02 | 0.23 | −2.50 |
| Cytoplasm_Texture_Entropy_Mito_20_01 | 2.65E−02 | 3.63E−02 | 0.21 | 1.61 |
| Cytoplasm_Texture_InfoMeas1_AGP_5_03 | 4.78E−02 | 3.71E−02 | 0.22 | −2.46 |
| Cytoplasm_Texture_InfoMeas2_Mito_5_00 | 1.37E−02 | 3.63E−02 | 0.30 | 2.06 |
| Cytoplasm_Texture_InverseDifferenceMoment_AGP_20_03 | 4.86E−02 | 3.75E−02 | 0.22 | 2.56 |
| Cytoplasm_Texture_InverseDifferenceMoment_Mito_20_01 | 2.47E−02 | 3.63E−02 | 0.24 | −1.79 |
| Cytoplasm_Texture_SumAverage_AGP_10_00 | 1.61E−02 | 3.63E−02 | 0.28 | −2.09 |
| Cytoplasm_Texture_SumEntropy_AGP_20_02 | 4.22E−02 | 3.63E−02 | 0.22 | −2.62 |
| Cytoplasm_Texture_SumEntropy_Mito_20_03 | 2.84E−02 | 3.63E−02 | 0.21 | 1.60 |
| Cytoplasm_Texture_SumVariance_AGP_10_02 | 3.24E−02 | 3.63E−02 | 0.24 | −2.36 |
| Cytoplasm_Texture_Variance_AGP_10_02 | 2.72E−02 | 3.63E−02 | 0.25 | −2.29 |

TABLE 18
Lists of pathways enriched among significant connections between gene and LP
features of rs12454712 profile. Term, which pathway; Overlap, number of genes that overlap
and total genes; P-value, enrichment p-value; Adjusted P-value, Q-value; Odds Ratio,
enrichment; Genes, genes in the pathway which are associated with gene LP-feature connections.

| | | | adj. | | |
|---|---|---|---|---|---|
| Term | Overlap | p-value | p-value | OR | Genes |
| fatty acid catabolic | 25/65 | 1.34E−07 | 5.63E−04 | 4.33 | PECR; ABCD2; ACAA2; ABHD3; ABHD1; HSD17B4; ACAT2; |
| process (GO: 0009062) | | | | | ACAT1; MCEE; HAO1; HADH; ACAD10; HIBCH; PHYH; |
| | | | | | GLYATL2; ACBD5; ADIPOQ; PEX13; HADHB; ALDH3A2; |
| | | | | | NUD17; PCCA; EHHADH; ADTRP; LPIN1 |
| fatty acid oxidation | 18/50 | 2.19E−05 | 3.47E−02 | 3.89 | PECR; ABCD2; PHYH; ACAA2; ADIPOQ; HSD17B4; ADIPOR2; |
| (GO: 0019395) | | | | | PEX13; ACAT2; ACAT1; HADHB; ALDH3A2; ACOX1; EHHADH; |
| | | | | | HAO1; HADH; ACAD10; HIBCH |
| branched-chain amino | 11/22 | 2.48E−05 | 3.47E−02 | 6.90 | MCCC2; GHR; STAT5B; ALDH6A1; HIBADH; IVD; BCKDHB; DBT; |
| acid metabolic process | | | | | HSD17B10; HIBCH; ACAT1 |
| (GO: 0009081) | | | | | |
| regulation of | 23/77 | 5.44E−05 | 5.71E−02 | 2.95 | SH3GLB1; BECN1; UVRAG; PLK3; PLK1; KIF14; KIF23; CDC6; |
| cytokinesis | | | | | RXFP3; CDC25B; CIT; AURKA; RACGAP1; PRC1; KIF13A; PKN2; |
| (GO:0032465) | | | | | KIF20A; ECT2; RAB11FIP3; OR2A4; E2F7; E2F8; BCL2L1 |
| fatty acid beta-oxidation | 17/50 | 8.50E−05 | 5.95E−02 | 3.56 | GCDH; ABCD2; ACAA2; ECI2; ADIPOQ; HSD17B4; ETFA; ACAT2; |
| (GO: 0006635) | | | | | ACAT1; HADHB; SCP2; ACOX1; EHHADH; IVD; HADH; ACAD10; |
| | | | | | HIBCH |
| regulation of cell cycle | 25/90 | 9.89E−05 | 5.95E−02 | 2.66 | BECN1; FOXM1; AURKA; RACGAP1; KIF13A; NEK2; FBXO5; |
| process (GO: 0010564) | | | | | ECT2; RAB11FIP3; SH3GLB1; PRMT5; UVRAG; PLK3; RMI2; |
| | | | | | YTHDF2; SPAG5; CAV2; PLK1; HMGA2; CDC25C; CDK5; PRC1; |
| | | | | | KIF20A; SLC25A33; BCL2L1 |
| branched-chain amino | 10/21 | 9.92E−05 | 5.95E−02 | 6.27 | MCCC2; HMGCL; ALDH6A1; HIBADH; IVD; BCKDHB; DBT; |
| acid catabolic process | | | | | HSD17B10; HIBCH; ACAT1 |
| (GO: 0009083) | | | | | |
| microtubule | 15/44 | 2.12E−04 | 9.73E−02 | 3.57 | GPSM2; STIL; DCTN2; PLK2; AURKA; CCNB1; ESPL1; KIF4A; |
| cytoskeleton | | | | | STMN1; NUSAP1; EFHC1; TACC3; RAN; CLASP2; SPC25 |
| organization involved in | | | | | |
| mitosis (GO: 1902850) | | | | | |
| negative regulation of | 9/19 | 2.35E−04 | 9.73E−02 | 6.21 | MRAP; DAB2; SAPCD2; TMBIM1; PPP2R5A; GBP1; MRAP2; |
| protein localization to | | | | | RHOQ; BCL2L1 |
| cell periphery | | | | | |
| (GO: 1904376) | | | | | |
| cellular amino acid | 10/23 | 2.53E−04 | 9.73E−02 | 5.31 | MCCC2; ALDH6A1; HIBADH; IVD; BCKDHB; DBT; ABAT; |
| catabolic process | | | | | HSD17B10; HIBCH; ACAT1 |
| (GO: 0009063) | | | | | |
| mitotic cytokinesis | 18/59 | 2.55E−04 | 9.73E−02 | 3.03 | PLK1; KIF23; SNX33; CENPA; CIT; ANLN; SNX18; ESPL1; |
| (GO: 0000281) | | | | | RACGAP1; INCENP; KIF4A; STMN1; NUSAP1; EFHC1; |
| | | | | | STAMBP; KIF20A; ECT2; CEP55 |
| cytoskeleton- | 20/70 | 3.16E−04 | 1.04E−01 | 2.76 | IST1; PLK1; KIF23; SNX33; CENPA; CIT; ANLN; SNX18; BIN3; |
| dependent cytokinesis | | | | | ESPL1; RACGAP1; INCENP; KIF4A; STMN1; NUSAP1; EFHC1; |
| (GO: 0061640) | | | | | STAMBP; KIF20A; ECT2; CEP55 |
| regulation of cell | 19/65 | 3.21E−04 | 1.04E−01 | 2.85 | SH3GLB1; BECN1; UVRAG; PLK3; BLM; SUSD2; PLK1; HTR2B; |
| division (GO: 0051302) | | | | | SIRT2; AURKA; KIF18B; PRC1; KIF13A; EFHC1; SFN; |
| | | | | | KIF20A; RAB11FIP3; FGFR2; BCL2L1 |
| regulation of | 6/10 | 5.53E−04 | 1.66E−01 | 10.34 | CCNB1; RACGAP1; SPAG5; NEK2; ECT2; SIRT2 |
| attachment of spindle | | | | | |
| microtubules to | | | | | |
| kinetochore | | | | | |
| (GO: 0051988) | | | | | |
| signal transduction | 18/63 | 6.19E−04 | 1.73E−01 | 2.76 | PLK3; CDKN1B; PCNA; PRMT1; PRKDC; PLK2; CDC25C; PML; |
| involved in mitotic G1 | | | | | AURKA; CCNB1; TFDP1; CHEK2; CENPJ; BAX; SFN; |
| DNA damage | | | | | ZNF385A; E2F7; E2F8 |
| checkpoint | | | | | |
| (GO: 0072431) | | | | | |
| negative regulation of | 11/30 | 7.27E−04 | 1.80E−01 | 3.99 | CDKN1C; PRDX3; PIF1; ZFP36; CDKN1B; BAG5; DTX3L; SRC; |
| transferase activity | | | | | POT1; MAPT; NIPSNAP2 |
| (GO: 0051348) | | | | | |
| positive regulation of | 26/107 | 7.28E−04 | 1.80E−01 | 2.22 | GPSM2; KIF14; RXFP3; AURKA; CCNB1; RACGAP1; SOX15; TPR; |
| cell cycle process | | | | | NUSAP1; PRKACA; ECT2; E2F7; E2F8; SPAG5; INSR; HMGA2; |
| (GO: 0090068) | | | | | CDC7; KIF23; CDC6; SIRT2; CDC25B; CIT; FAP; MYBBP1A; |
| | | | | | PKN2; OR2A4 |
| regulation of | 10/26 | 8.19E−04 | 1.87E−01 | 4.31 | DHFR; CDT1; PCNA; TFDP1; ORC1; FBXO5; CDC6; TYMS; E2F7; |
| transcription involved in | | | | | ZPR1 |
| G1/S transition of | | | | | |
| mitotic cell cycle | | | | | |
| (GO: 0000083) | | | | | |
| cholesterol biosynthetic | 12/35 | 8.47E−04 | 1.87E−01 | 3.60 | IDI1; FDPS; ACLY; PLPP6; EBP; MVK; ACAA2; PMVK; SC5D; |
| process (GO: 0006695) | | | | | MSMO1; DHCR7; ACAT2 |
| sterol biosynthetic | 13/40 | 9.27E−04 | 1.94E−01 | 3.32 | IDI1; FDPS; MVK; ACAA2; MSMO1; ACAT2; ACAT1; ACLY; |
| process (GO: 0016126) | | | | | PLPP6; EBP; PMVK; SC5D; DHCR7 |
| positive regulation of | 21/82 | 1.11E−03 | 2.15E−01 | 2.38 | PLK3; CDKN1B; PCNA; PRMT1; PLK2; HMGA2; CDC25C; PML; |
| cell cycle arrest | | | | | AURKA; CCNB1; TFDP1; FAP; MYBBP1A; CHEK2; CENPJ; BAX; |
| (GO:0071158) | | | | | SFN; PRKACA; ZNF385A; E2F7; E2F8 |
| secondary alcohol | 12/36 | 1.12E−03 | 2.15E−01 | 3.45 | IDI1; FDPS; ACLY; PLPP6; EBP; MVK; ACAA2; PMVK; MSMO1; |
| biosynthetic process | | | | | DHCR7; ACAT2; ACAT1 |
| (GO: 1902653) | | | | | |
| positive regulation of | 13/41 | 1.20E−03 | 2.19E−01 | 3.20 | KIF14; HTR2B; KIF23; CDC6; SIRT2; RXFP3; CIT; CDC25B; |
| cell division | | | | | RACGAP1; PKN2; ECT2; OR2A4; FGFR2 |
| (GO: 0051781) | | | | | |
| fatty acid alpha- | 5/8 | 1.32E−03 | 2.22E−01 | 11.48 | ALDH3A2; PECR; PHYH; HAO1; PEX13 |
| oxidation (GO: 0001561) | | | | | |
| ribosome biogenesis | 45/226 | 1.36E−03 | 2.22E−01 | 1.72 | RPL3; DDX27; PAK1IP1; NIP7; DIS3L; RPLPO; MALSU1; RPL10A; |
| (GO: 0042254) | | | | | WDR46; RPL6; WDR43; RPL7; NOL6; EXOSC7; RPS14; FBL; |
| | | | | | RPL7A; PDCD11; NOB1; RPL14; RPS3; RPL15; UTP14A; EXOSC2; |
| | | | | | MDN1; DCAF13; NOP56; NOP58; NOP16; UTP3; TEX10; RPS5; |
| | | | | | DDX56; DDX31; RPL13A; RPSA; RPL23A; WDR12; GNL1; LSG1; |
| | | | | | XRN2; TSR1; ABCE1; RAN; RPS23 |
| negative regulation of | 8/19 | 1.37E−03 | 2.22E−01 | 5.01 | MRAP; DAB2; TMBIM1; PPP2R5A; GBP1; MRAP2; RHOQ; |
| protein localization to | | | | | BCL2L1 |
| plasma membrane | | | | | |
| (GO: 1903077) | | | | | |
| DNA damage response, | 17/62 | 1.44E−03 | 2.23E−01 | 2.61 | PLK3; CDKN1B; PCNA; PRMT1; PLK2; CDC25C; PML; AURKA; |
| signal transduction by | | | | | CCNB1; TFDP1; CHEK2; CENPJ; BAX; SFN; ZNF385A; E2F7; E2F8 |
| p53 class mediator | | | | | |
| resulting in cell cycle | | | | | |
| arrest (GO: 0006977) | | | | | |
| rRNA metabolic process | 40/200 | 2.23E−03 | 3.34E−01 | 1.73 | RPL3; DDX27; DIS3L; RPLPO; RPL10A; WDR46; RPL6; WDR43; |
| (GO: 0016072) | | | | | RPL7; NOL6; EXOSC7; RPS14; FBL; RPL7A; PDCD11; NOB1; |
| | | | | | RPL14; RPS3; RPL15; UTP14A; EXOSC2; MDN1; DCAF13; NOP56; |
| | | | | | NOP58; NIFK; UTP3; TEX10; RPS5; DDX56; CD3EAP; RPL13A; |
| | | | | | RPSA; RPL23A; WDR12; XRN2; TSR1; ANG; MAPT; RPS23 |
| embryonic skeletal | 8/21 | 2.93E−03 | 3.75E−01 | 4.24 | KIAA1217; WNT11; SIX1; SOX11; WNT9A; HOXD3; MEGF8; |
| system development | | | | | DSCAML1 |
| (GO: 0048706) | | | | | |
| regulation of | 12/40 | 3.09E−03 | 3.75E−01 | 2.96 | IDI1; FDPS; MVK; GPAM; PMVK; SC5D; SCAP; LipidLIN2; |
| cholesterol biosynthetic | | | | | DHCR7; ABCG1; RAN; SOD1 |
| process (GO: 0045540) | | | | | |
| pyrimidine-containing | 4/6 | 3.14E−03 | 3.75E−01 | 13.77 | SLC19A3; C2ORF83; SLC25A33; SLC25A36 |
| compound | | | | | |
| transmembrane | | | | | |
| transport (GO: 0072531) | | | | | |
| regulation of protein | 4/6 | 3.14E−03 | 3.75E−01 | 13.77 | GPSM2; SAPCD2; EPB41; GNAI1 |
| localization to cell | | | | | |
| cortex (GO: 1904776) | | | | | |
| succinyl-CoA metabolic | 4/6 | 3.14E−03 | 3.75E−01 | 13.77 | DLST; SUCLG2; SUCLG1; ACOT4 |
| process (GO: 0006104) | | | | | |
| fatty acid metabolic | 24/106 | 3.15E−03 | 3.75E−01 | 2.02 | STAT5B; PHYH; ACBD5; ABCD2; GPX4; ACSL1; PNPLA8; ADIPOQ; |
| process (GO: 0006631) | | | | | ACSM6; ACSM5; MSMO1; ALKBH7; ADIPOR2; GHR; ACLY; |
| | | | | | GNPAT; NUDT7; GPAM; ACOX1; PNPLA3; GPAT2; LPIN1; |
| | | | | | ACOT4; CBR4 |
| 2-oxoglutarate | 7/17 | 3.21E−03 | 3.75E−01 | 4.82 | GHR; STAT5B; PHYH; MRPS36; IDH1; GPT2; DLST |
| metabolic process | | | | | |
| (GO: 0006103) | | | | | |
| alpha-linolenic acid | 6/13 | 3.22E−03 | 3.75E−01 | 5.91 | FADS2; SCP2; ELOVL5; ACSL1; ACOX1; HSD17B4 |
| metabolic process | | | | | |
| (GO: 0036109) | | | | | |
| tRNA methylation | 9/26 | 3.47E−03 | 3.90E−01 | 3.65 | TRMT10A; METTL1; THUMPD2; TRMT1; THADA; MTO1; |
| (GO: 0030488) | | | | | HSD17B10; FTSJ1; TRMT61A |
| positive regulation of | 17/67 | 3.53E−03 | 3.90E−01 | 2.35 | GPSM1; SH3GLB1; PRKN; BNIP3L; PLK3; SCOC; PLK2; TFEB; |
| autophagy | | | | | PIK3CB; TICAM1; SUPT5H; SVIP; MID2; DCN; PRKD1; TRIM21; |
| (GO: 0010508) | | | | | TRIM22 |
| protein tetramerization | 21/90 | 3.80E−03 | 4.01E−01 | 2.10 | GBP5; CD74; UXS1; HM13; INSR; APIP; ACOT13; SAMHD1; |
| (GO: 0051262) | | | | | HSD17B10; THG1L; CRYZ; DHRS4; HMGCL; SRR; RXRA; GPRIN1; |
| | | | | | CAT; CD247; KCNJ2; CBR4; S100A10 |
| positive regulation of | 16/62 | 3.82E−03 | 4.01E−01 | 2.40 | CREBBP; DHX9; PRKDC; XRCC5; PTPN22; RELA; DHX33; IFI16; |
| type I interferon | | | | | IRAK1; POLR3C; IRF1; POLR1C; TLR9; STAT6; POLR3H; MYD88 |
| production | | | | | |
| (GO: 0032481) | | | | | |
| STAT cascade | 8/22 | 4.09E−03 | 4.09E−01 | 3.94 | GHR; STAT5B; STAT2; CLCF1; CTR9; CCL2; NMI; STAMBP |
| (GO: 0097696) | | | | | |
| protein sumoylation | 17/68 | 4.16E−03 | 4.09E−01 | 2.30 | TOP2A; NUP214; NUP205; PCNA; L3MBTL2; UBE21; BCL11A; |
| (GO: 0016925) | | | | | NUP153; AAAS; PIAS2; SENP1; NUP93; SUMO1; INCENP; TPR; |
| | | | | | BIRC5; NUP98 |
| protein modification by | 69/398 | 4.24E−03 | 4.09E−01 | 1.45 | TOP2A; DET1; RNF14; FBXO25; KLHDC7A; HIF3A; UBE2Z; CDC73; |
| small protein | | | | | HLipidC4; HLipidC3; SUMO1; TRIM21; FBXO9; TRIM23; NUP214; |
| conjugation | | | | | ATG10; FBXO10; PIAS2; CCNB1IP1; PJA1; SENP1; NUP93; |
| (GO: 0032446) | | | | | UBE2R2; INCENP; KCTD10; BIRC5; NUP98; PRKN; NUP205; |
| | | | | | RNF34; L3MBTL2; PCNA; ANAPC16; DCUN1D5; PRKDC; CTR9; |
| | | | | | CUL1; DLipidL1; KLHL10; UBR3; WDR61; NEDD8; BCL10; AAAS; |
| | | | | | UBE2J2; RNF4; HIF1A; TPR; HLTF; TMEM129; VHL; BARD1; |
| | | | | | BUE2H; UBE21; SMURF2; BCL11A; UBE2C; PLK1; KLHDC8B; |
| | | | | | NUP153; KLHL24; UBE2W; UBE2T; KLHL8; UBA1; FBXL5; |
| | | | | | KBTBD7; RNF220; NFE2L2 |
| mitotic spindle | 18/74 | 4.44E−03 | 4.09E−01 | 2.22 | GPSM2; ARHGEF10; STIL; DCTN2; PLK2; KIF23; AURKA; TPX2; |
| organization | | | | | CCNB1; PRC1; CHEK2; KIF4A; STMN1; EFHC1; BIRC5; RAN; |
| (GO: 0007052) | | | | | CLASP2; SPC25 |
| ribosomal large subunit | 16/63 | 4.53E−03 | 4.09E−01 | 2.35 | RPL3; NOP16; PAK1IP1; NIP7; NOP2; RPL23A; MALSU1; RPL1OA; |
| biogenesis | | | | | WDR12; RPL6; NLE1; RPL7; RPL7A; RPL14; BRIX1; MDN1 |
| (GO: 0042273) | | | | | |
| cellular response to | 25/115 | 4.61E−03 | 4.09E−01 | 1.92 | VKORC1L1; TMEM161A; NCF2; PXN; HIF1A; EGFR; PRDX3; PRDX2; |
| oxidative stress | | | | | MAPK9; PRDX5; TXNDC8; BRF2; TXNRD1; GSR; CYBA; PYCR2; |
| (GO: 0034599) | | | | | WNT16; ETV5; SIRT2; SOD1; PXMP2; CAT; PRKD1; MAPT; |
| | | | | | NFE2L2 |
| mitotic cell cycle phase | 42/221 | 4.65E−03 | 4.09E−01 | 1.62 | CDKN1B; DCTN2; CCNH; CEP164; CUL1; FOXM1; AURKA; PPP6C; |
| transition (GO: 0044772) | | | | | CCNB1; TUBA1A; ORC1; PRKAR2B; CHEK2; NEK2; PRKACA; |
| | | | | | RAB8A; YWHAG; CLASP2; ACVR1; PLK4; PLK3; CDT1; UBE2C; |
| | | | | | ODF2; PLK2; TUBB; PLK1; CDC7; CDC6; CDC25C; CKAP5; |
| | | | | | CDC25B; POLA2; CDK7; MELK; CENPJ; POLE2; PPP2R2D; |
| | | | | | TACC3; MCM4; CACUL1; CDKN3 |
| modulation by virus of | 7/18 | 4.68E−03 | 4.09E−01 | 4.39 | SMAD3; RXRA; VAPB; INSR; KPNA7; KPNA5; KPNA2 |
| host morphology or | | | | | |
| physiology | | | | | |
| (GO: 0019048) | | | | | |
| sister chromatid | 10/32 | 4.87E−03 | 4.14E−01 | 3.13 | TOP2A; HIRA; KIF18B; SPAG5; ESPL1; PLK1; NUSAP1; |
| segregation | | | | | CHAMP1; NSL1; RAN |
| (GO: 0000819) | | | | | |
| ncRNA transcription | 6/14 | 5.03E−03 | 4.14E−01 | 5.17 | NIFK; SNAPC3; SNAPC4; CD3EAP; ANG; RPAP2 |
| (GO: 0098781) | | | | | |
| regulation of | 6/14 | 5.03E−03 | 4.14E−01 | 5.17 | NUP214; IFI27; MX2; XPO5; AAAS; UHMK1 |
| nucleocytoplasmic | | | | | |
| transport (GO: 0046822) | | | | | |
| DNA damage response, | 19/82 | 6.20E−03 | 4.71E−01 | 2.08 | PLK3; SP100; CDKN1B; PCNA; PRMT1; PLK2; FOXM1; CDC25C; |
| signal transduction by | | | | | PML; AURKA; CCNB1; TFDP1; CHEK2; CENPJ; BAX; SFN; |
| p53 class mediator | | | | | ZNF385A; E2F7; E2F8 |
| (GO: 0030330) | | | | | |
| mitotic sister chromatid | 19/82 | 6.20E−03 | 4.71E−01 | 2.08 | BECN1; SPAG5; PLK1; KIF14; KIF23; KIF22; NCAPH; NSL1; HIRA; |
| segregation | | | | | CCNB1; KIF18B; ESPL1; PRC1; NUSAP1; PHF13; CHMP4A; |
| (GO: 0000070) | | | | | NCAPD2; DLGAP5; RAN |
| embryonic organ | 10/33 | 6.20E−03 | 4.71E−01 | 3.00 | ACVR1; EFEMP1; MFAP2; RBPMS2; SIX1; SOX11; HOXD3; |
| morphogenesis | | | | | MEGF8; DSCAML1; FGFR2 |
| (GO: 0048562) | | | | | |
| valine metabolic | 4/7 | 6.59E−03 | 4.71E−01 | 9.18 | GHR; STAT5B; ALDH6A1; HIBADH |
| process (GO: 0006573) | | | | | |
| oxaloacetate metabolic | 4/7 | 6.59E−03 | 4.71E−01 | 9.18 | GHR; STAT5B; ACLY; MDH1 |
| process (GO: 0006107) | | | | | |
| unsaturated fatty acid | 12/44 | 7.22E−03 | 4.71E−01 | 2.59 | FADS3; CYP2J2; FADS2; SCP2; ELOVL5; PNPLA8; ACSL1; |
| metabolic process | | | | | ACOX1; SCD5; HSD17B4; CYP1B1; DAGLB |
| (GO: 0033559) | | | | | |
| viral transcription | 24/113 | 7.34E−03 | 4.71E−01 | 1.86 | NUP214; NUP205; RPL3; DHX9; RPS5; RPLPO; RPL13A; RPSA; |
| (GO: 0019083) | | | | | NUP153; RPL23A; RPL10A; AAAS; USF2; RPL6; RPL7; NUP93; |
| | | | | | RPS14; RPL7A; TPR; RPL14; RPS3; NUP98; RPL15; RPS23 |
| negative regulation of | 8/24 | 7.47E−03 | 4.71E−01 | 3.45 | MRAP; DAB2; TMBIM1; PPP2R5A; MRAP2; GBP1; RHOQ; |
| protein localization to | | | | | BCL2L1 |
| membrane | | | | | |
| (GO: 1905476) | | | | | |
| regulation of mitotic | 6/15 | 7.49E−03 | 4.71E−01 | 4.59 | PTTG1; UBE2C; PLK1; TPR; TACC3; CDC6 |
| sister chromatid | | | | | |
| separation | | | | | |
| (GO: 0010965) | | | | | |
| long-chain fatty acid | 14/55 | 7.49E−03 | 4.71E−01 | 2.36 | CYP2J2; GLYATL2; ACSL1; ELOVL5; PNPLA8; HSD17B4; FADS2; |
| metabolic process | | | | | SCP2; ACOX1; PNPLA3; CYP1B1; ADTRP; DAGLB; ACOT4 |
| (GO: 0001676) | | | | | |
| acyl-CoA metabolic | 11/39 | 7.58E−03 | 4.71E−01 | 2.71 | MCEE; DBT; DLST; ACSM6; ACOT13; SUCLG2; DBI; ACSM5; |
| process (GO: 0006637) | | | | | SUCLG1; GLYAT; ACOT4 |
| positive regulation of I- | 32/163 | 7.66E−03 | 4.71E−01 | 1.69 | PRKN; ECM1; GPR89A; SLC44A2; SLC20A1; TRADD; HTR2B; |
| kappaB kinase/NF- | | | | | BCL10; ATP2C1; EDA2R; RELA; IKBKB; IRAK1; TNFSF10; TRIM25; |
| kappaB signaling | | | | | ECT2; TRIM22; APOL3; MAP3K3; CD74; TRIM62; NDFIP1; PLK2; |
| (GO: 0043123) | | | | | F2R; ADIPOQ; TICAM1; MID2; TNIP2; TLR9; PRKD1; LTBR; |
| | | | | | MYD88 |
| positive regulation of | 10/34 | 7.81E−03 | 4.71E−01 | 2.87 | RACGAP1; KIF14; KIF23; PKN2; CDC6; ECT2; OR2A4; RXFP3; |
| cytokinesis | | | | | CIT; CDC25B |
| (GO: 0032467) | | | | | |
| positive regulation of | 5/11 | 7.82E−03 | 4.71E−01 | 5.74 | ZFP36; DHX9; XPO5; LIMD1; PUM2 |
| posttranscriptional | | | | | |
| gene silencing | | | | | |
| (GO: 0060148) | | | | | |
| regulation of | 5/11 | 7.82E−03 | 4.71E−01 | 5.74 | CCNK; CCNT1; CCNH; CCNL2; PPIL4 |
| phosphorylation of RNA | | | | | |
| polymerase II C- | | | | | |
| terminal domain | | | | | |
| (GO: 1901407) | | | | | |
| negative regulation of | 5/11 | 7.82E−03 | 4.71E−01 | 5.74 | BARD1; SP100; MDFIC; TPR; NUP153 |
| nucleocytoplasmic | | | | | |
| transport (GO: 0046823) | | | | | |
| magnesium ion | 5/11 | 7.82E−03 | 4.71E−01 | 5.74 | NIPAL4; MRS2; TUSC3; NIPAL2; SLC41A1 |
| transport (GO: 0015693) | | | | | |
| regulation of | 5/11 | 7.82E−03 | 4.71E−01 | 5.74 | SYDE1; ACVR1C; SMURF2; TIMP1; NODAL |
| trophoblast cell | | | | | |
| migration | | | | | |
| (GO: 1901163) | | | | | |
| positive regulation of | 54/307 | 7.85E−03 | 4.71E−01 | 1.48 | TOP2A; NDUFA13; MTCH2; TRIO; TRADD; PCSK9; UBE2Z; IFIT2; |
| apoptotic process | | | | | RASSF2; TCTN3; TNFSF10; CYP1B1; UNC13B; VAV3; BNIP3L; |
| (GO: 0043065) | | | | | ADIPOQ; F2R; DUSP6; SIRT2; VAV2; CLIP3; MELK; SHQ1; SIK1; |
| | | | | | SOS1; NGEF; BCL2A1; BCL10; APH1A; MAPK9; BCL2L11; ACVR1C; |
| | | | | | PNMA1; FRZB; RPS3; PMAIP1; ECT2; ZNF268; BARD1; IFNB1; |
| | | | | | SIAH1; DKKL1; HMGA2; GADD45G; SOD1; IL6; MYBBP1A; |
| | | | | | FAP; CDK5; BCL6; FAS; BAX; BLID; NODAL |
| cell cycle G2/M phase | 26/127 | 8.87E−03 | 5.24E−01 | 1.78 | DCTN2; CCNH; CEP164; CUL1; AKAP8; FOXM1; AURKA; CCNB1; |
| transition (GO: 0044839) | | | | | TUBA1A; PRKAR2B; CHEK2; NEK2; PRKACA; YWHAG; RAB8A; |
| | | | | | PLK4; PLK3; ODF2; TUBB; PLK1; CDC25C; CKAP5; CDC25B; |
| | | | | | CDK7; MELK; CENPJ |
| regulation of type I | 19/85 | 9.24E−03 | 5.39E−01 | 1.99 | CREBBP; DHX9; PRKDC; XRCC5; TNFAIP3; PTPN22; UBE2L6; |
| interferon production | | | | | RELA; DHX33; IFI16; IRAK1; POLR3C; IRF1; POLR1C; TRIM25; |
| (GO: 0032479) | | | | | STAT6; POLR3H; TRIM21; MYD88 |
| coenzyme metabolic | 13/51 | 9.66E−03 | 5.55E−01 | 2.36 | PANK1; ACSM6; ACOT13; DBI; ACSM5; GLYAT; ACAT1; |
| process (GO: 0006732) | | | | | SLC5A6; SLC25A16; ACLY; VNN1; DBT; ACOT4 |
| fatty-acyl-CoA | 9/30 | 9.92E−03 | 5.63E−01 | 2.95 | ACLY; ELOVL5; ACSL1; SCD5; ACSL4; ACSL3; HACD2; CBR4; |
| biosynthetic process | | | | | ACAT1 |
| (GO: 0046949) | | | | | |
| cellular response to | 23/110 | 1.03E−02 | 5.74E−01 | 1.83 | PRKDC; SRC; INSR; SYAP1; ADIPOQ; DENND4C; PDE3B; PCSK9; |
| peptide hormone | | | | | ADCY3; SORBS1; ADCY8; RELA; ADCY5; KAT2B; ADCY9; GNG2; |
| stimulus (GO: 0071375) | | | | | TBC1D4; PRKAR2B; PRKACA; SLC25A33; RAB8A; VAMP2; |
| | | | | | RHOQ |
| protein | 14/57 | 1.04E−02 | 5.74E−01 | 2.25 | GBP5; HM13; APIP; ACOT13; SAMHD1; HSD17B10; THG1L; |
| homotetramerization | | | | | CRYZ; SRR; RXRA; CAT; CD247; KCNJ2; CBR4 |
| (GO: 0051289) | | | | | |
| activation of protein | 6/16 | 1.07E−02 | 5.84E−01 | 4.13 | ADCY9; PRKAR2B; ADCY3; ADCY8; PRKACA; ADCY5 |
| kinase A activity | | | | | |
| (GO: 0034199) | | | | | |
| regulation of innate | 16/69 | 1.14E−02 | 5.95E−01 | 2.08 | GBP5; DHX9; IFNB1; STAT2; IFNA2; TNFAIP3; PTPN22; SAMHD1; |
| immune response | | | | | IFNA8; FGR; IFI16; POLR3C; IRF1; TYRO3; CD36; ABCE1 |
| (GO: 0045088) | | | | | |
| G1/S transition of | 22/105 | 1.17E−02 | 5.95E−01 | 1.83 | ACVR1; PLK3; CDT1; CDKN1B; PCNA; CCNH; PLK2; CUL1; CDC7; |
| mitotic cell cycle | | | | | CDC6; TYMS; DHFR; PPP6C; POLA2; CDK7; TFDP1; ORC1; POLE2; |
| (GO: 0000082) | | | | | MCM4; FBXO5; CACUL1; CDKN3 |
| protein K6-linked | 4/8 | 1.19E−02 | 5.95E−01 | 6.89 | PRKN; BARD1; UBE2T; RNF4 |
| ubiquitination | | | | | |
| (GO: 0085020) | | | | | |
| angiogenesis involved | 4/8 | 1.19E−02 | 5.95E−01 | 6.89 | MCAM; NDNF; HPSE; ADIPOR2 |
| in wound healing | | | | | |
| (GO: 0060055) | | | | | |
| positive regulation of | 4/8 | 1.19E−02 | 5.95E−01 | 6.89 | CD74; DCSTAMP; CSF1; CTNNBIP1 |
| monocyte | | | | | |
| differentiation | | | | | |
| (GO: 0045657) | | | | | |
| signal transduction | 5/12 | 1.20E−02 | 5.95E−01 | 4.92 | BABAM2; ABRAXAS1; PLK1; CHEK1; UIMC1 |
| involved in G2 DNA | | | | | |
| damage checkpoint | | | | | |
| (GO: 0072425) | | | | | |
| mitochondrial outer | 5/12 | 1.20E−02 | 5.95E−01 | 4.92 | BNIP3L; RHOT1; BCL2L11; BAX; PMAIP1 |
| membrane | | | | | |
| permeabilization | | | | | |
| (GO: 0097345) | | | | | |
| embryonic skeletal | 7/21 | 1.21E−02 | 5.95E−01 | 3.45 | EIF4A3; SIX1; SOX11; HOXD3; MEGF8; DSCAML1; FGFR2 |
| system morphogenesis | | | | | |
| (GO: 0048704) | | | | | |
| ncRNA processing | 41/227 | 1.22E−02 | 5.95E−01 | 1.52 | RPL3; DDX27; POP1; DIS3L; RPLPO; ADAR; RPL10A; WDR46; RPL6; |
| (GO: 0034470) | | | | | WDR43; RPL7; NOL6; THG1L; EXOSC7; RPS14; FBL; RPL7A; |
| | | | | | PDCD11; NOB1; RPL14; RPS3; RPL15; UTP14A; EXOSC2; MDN1; |
| | | | | | DCAF13; SMAD2; NOP56; NOP58; SMAD3; UTP3; TEX10; RPS5; |
| | | | | | DDX56; RPL13A; RPSA; RPL23A; WDR12; XRN2; TSR1; RPS23 |
| regulation of protein | 9/31 | 1.24E−02 | 6.00E−01 | 2.82 | BARD1; SP100; IFI27; TPR; XPO5; CAMK1; SUPT6H; UHMK1; |
| export from nucleus | | | | | RBM22 |
| (GO: 0046825) | | | | | |
| negative regulation of | 19/88 | 1.34E−02 | 6.25E−01 | 1.90 | YTHDF2; EIF4A3; SAMD4A; RPL13A; SAMD4B; MALSU1; LIMD1; |
| translation | | | | | TYMS; DHFR; MOV10; METTL14; PAIP2B; ILF3; RIDA; TPR; |
| (GO: 0017148) | | | | | RPS3; EIF4EBP2; PAIP2; CPEB3 |
| positive regulation of | 13/53 | 1.34E−02 | 6.25E−01 | 2.24 | SMAD3; JUP; F2R; EGFR; NUP93; IL6; DAB2; TPR; HCLS1; ECT2; |
| protein import into | | | | | NODAL; RBM22; ZPR1 |
| nucleus (GO: 0042307) | | | | | |
| rRNA processing | 37/202 | 1.34E−02 | 6.25E−01 | 1.55 | RPL3; DDX27; DIS3L; RPLPO; NOP2; RPL10A; WDR46; RPL6; |
| (GO: 0006364) | | | | | WDR43; RPL7; NOL6; EXOSC7; RPS14; FBL; RPL7A; PDCD11; |
| | | | | | NOB1; RPL14; RPS3; RPL15; UTP14A; EXOSC2; MDN1; DCAF13; |
| | | | | | NOP56; NOP58; UTP3; TEX10; RPS5; DDX56; RPL13A; RPSA; |
| | | | | | RPL23A; WDR12; XRN2; TSR1; RPS23 |
| positive regulation of | 11/42 | 1.36E−02 | 6.25E−01 | 2.45 | JUP; DTX3L; PYHIN1; TPR; PLK1; LIF; KAT7; ECT2; CCT5; RBM22; |
| protein localization to | | | | | ZPR1 |
| nucleus (GO: 1900182) | | | | | |
| dicarboxylic acid | 14/59 | 1.41E−02 | 6.43E−01 | 2.15 | STAT5B; PHYH; MDH1; MRPS36; GPT2; IDH1; DLST; GHR; DHFR; |
| metabolic process | | | | | ACLY; MTHFD1; ME1; SUCLG1; ACOT4 |
| (GO: 0043648) | | | | | |
| cellular response to | 15/65 | 1.46E−02 | 6.51E−01 | 2.07 | IFITM3; SP100; IFNB1; MX2; STAT2; IFNA2; ADAR; SAMHD1; |
| type I interferon | | | | | IFNA8; IFIT3; IFIT2; IFI27; IRAK1; IRF1; MYD88 |
| (GO: 0071357) | | | | | |
| type I interferon | 15/65 | 1.46E−02 | 6.51E−01 | 2.07 | IFITM3; SP100; IFNB1; MX2; STAT2; IFNA2; ADAR; SAMHD1; |
| signaling pathway | | | | | IFNA8; IFIT3; IFIT2; IFI27; IRAK1; IRF1; MYD88 |
| (GO: 0060337) | | | | | |
| positive regulation of | 6/17 | 1.48E−02 | 6.52E−01 | 3.76 | HEYL; KAT2B; CREBBP; NOTCH4; RBPJ; DLGAP5 |
| transcription of Notch | | | | | |
| receptor target | | | | | |
| (GO: 0007221) | | | | | |
| G2/M transition of | 25/126 | 1.49E−02 | 6.52E−01 | 1.71 | DCTN2; CCNH; CEP164; CUL1; FOXM1; AURKA; CCNB1; |
| mitotic cell cycle | | | | | TUBA1A; PRKAR2B; CHEK2; NEK2; PRKACA; YWHAG; RAB8A; |
| (GO: 0000086) | | | | | PLK4; PLK3; ODF2; TUBB; PLK1; CDC25C; CKAP5; CDC25B; |
| | | | | | CDK7; MELK; CENPJ |
| endodermal cell | 9/32 | 1.54E−02 | 6.57E−01 | 2.70 | COL4A2; CTR9; HMGA2; ITGA7; ITGA5; MMP8; CDC73; |
| differentiation | | | | | NODAL; MIXL1 |
| (GO: 0035987) | | | | | |
| G2 DNA damage | 9/32 | 1.54E−02 | 6.57E−01 | 2.70 | FANCI; BABAM2; BLM; ABRAXAS1; CHEK1; UIMC1; PLK1; |
| checkpoint | | | | | HMGA2; FOXN3 |
| (GO: 0031572) | | | | | |
| RNA modification | 13/54 | 1.57E−02 | 6.57E−01 | 2.19 | TRUB1; NOP56; NOP58; METTL1; NOP2; MEPCE; ADAR; TRMT1; |
| (GO: 0009451) | | | | | FTSJ1; C9ORF64; THG1L; TRMT61A; NOP10 |
| sulfur amino acid | 7/22 | 1.59E−02 | 6.57E−01 | 3.22 | MSRA; NFS1; AHCY; MTHFD1; CBS; BHMT2; PCYOX1 |
| metabolic process | | | | | |
| (GO: 0000096) | | | | | |
| ciliary basal body- | 20/96 | 1.67E−02 | 6.57E−01 | 1.82 | PLK4; DCTN2; ODF2; TUBB; CEP164; PLK1; KIF24; RPGRIP1L; |
| plasma membrane | | | | | CKAP5; AHI1; TUBA1A; TCTN3; PRKAR2B; CENPJ; NEK2; B9D2; |
| docking (GO: 0097711) | | | | | PRKACA; YWHAG; RAB8A; CEP89 |
| positive regulation of | 15/66 | 1.67E−02 | 6.57E−01 | 2.03 | PRKN; CDT1; PLK3; RIPOR1; DTX3L; PYHIN1; PLK1; LIF; SNX33; |
| cellular protein | | | | | TM9SF4; TPR; KAT7; MAPT; CENPQ; VAMP2 |
| localization | | | | | |
| (GO: 1903829) | | | | | |
| regulation of cell cycle | 18/84 | 1.70E−02 | 6.57E−01 | 1.88 | PLK4; DCTN2; ODF2; TUBB; CEP164; PLK1; KIF14; HMMR; CKAP5; |
| G2/M phase transition | | | | | AURKA; TPX2; TUBA1A; PRKAR2B; CENPJ; NEK10; NEK2; |
| (GO: 1902749) | | | | | PRKACA; YWHAG |
| RNA methylation | 12/49 | 1.74E−02 | 6.57E−01 | 2.24 | FBL; TRMT10A; METTL14; METTL1; TGS1; NOP2; MEPCE; |
| (GO: 0001510) | | | | | THUMPD2; THADA; MTO1; FTSJ1; TRMT61A |
| positive regulation of | 12/49 | 1.74E−02 | 6.57E−01 | 2.24 | BABAM2; SPRED2; EID3; PCNA; TMEM161A; DHX9; |
| response to DNA | | | | | ABRAXAS1; UIMC1; RPS3; PMAIP1; HMGA2; EGFR |
| damage stimulus | | | | | |
| (GO: 2001022) | | | | | |
| positive regulation of | 5/13 | 1.75E−02 | 6.57E−01 | 4.30 | DNMT1; AUTS2; KMT2A; CTR9; WDR61 |
| histone H3-K4 | | | | | |
| methylation | | | | | |
| (GO: 0051571) | | | | | |
| signal transduction | 5/13 | 1.75E−02 | 6.57E−01 | 4.30 | BABAM2; ABRAXAS1; PLK1; CHEK1; UIMC1 |
| involved in DNA | | | | | |
| damage checkpoint | | | | | |
| (GO: 0072422) | | | | | |
| multivesicular body | 5/13 | 1.75E−02 | 6.57E−01 | 4.30 | UVRAG; SORT1; RAB27A; RAB27B; SYTL4 |
| sorting pathway | | | | | |
| (GO: 0071985) | | | | | |
| placenta development | 5/13 | 1.75E−02 | 6.57E−01 | 4.30 | ANG; NODAL; E2F7; SOD1; E2F8 |
| (GO: 0001890) | | | | | |
| modulation by virus of | 5/13 | 1.75E−02 | 6.57E−01 | 4.30 | PRKN; CCNK; KPNA7; KPNA5; KPNA2 |
| host process | | | | | |
| (GO: 0019054) | | | | | |
| Notch signaling | 13/55 | 1.82E−02 | 6.57E−01 | 2.13 | CREBBP; NOTCH4; DTX1; NRARP; RBPJ; NLE1; APH1A; KAT2B; |
| pathway (GO: 0007219) | | | | | HEYL; GMDS; SNAI1; HOXD3; DLGAP5 |
| tRNA-containing | 9/33 | 1.89E−02 | 6.57E−01 | 2.58 | NUP214; NUP93; NUP205; TPR; NUP153; NUP98; AAAS; RAN; |
| ribonucleoprotein | | | | | NOL6 |
| complex export from | | | | | |
| nucleus {GO: 0071431) | | | | | |
| tRNA export from | 9/33 | 1.89E−02 | 6.57E−01 | 2.58 | NUP214; NUP93; NUP205; TPR; NUP153; NUP98; AAAS; RAN; |
| nucleus (GO: 0006409) | | | | | NOL6 |
| secretory granule | 4/9 | 1.92E−02 | 6.57E−01 | 5.51 | SRGN; F2R; LYST; F2RL3 |
| organization | | | | | |
| (GO: 0033363) | | | | | |
| mitochondrial | 4/9 | 1.92E−02 | 6.57E−01 | 5.51 | UQCC1; LYRM7; UQCR10; SLC25A33 |
| respiratory chain | | | | | |
| complex III assembly | | | | | |
| (GO: 0034551) | | | | | |
| respiratory chain | 4/9 | 1.92E−02 | 6.57E−01 | 5.51 | UQCC1; LYRM7; UQCR10; SLC25A33 |
| complex III assembly | | | | | |
| (GO: 0017062) | | | | | |
| positive regulation of | 4/9 | 1.92E−02 | 6.57E−01 | 5.51 | CCNK; CCNT1; CCNH; CCNL2 |
| phosphorylation of RNA | | | | | |
| polymerase II C- | | | | | |
| terminal domain | | | | | |
| (GO: 1901409) | | | | | |
| negative regulation of | 4/9 | 1.92E−02 | 6.57E−01 | 5.51 | CYP27B1; SCAP; LipidLIN2; SOD1 |
| alcohol biosynthetic | | | | | |
| process (GO: 1902931) | | | | | |
| histone H3-K4 | 4/9 | 1.92E−02 | 6.57E−01 | 5.51 | KMT2A; CTR9; WDR61; ZNF335 |
| trimethylation | | | | | |
| (GO: 0080182) | | | | | |
| negative regulation of | 4/9 | 1.92E−02 | 6.57E−01 | 5.51 | BARD1; CCNT1; CTR9; SUPT5H |
| mRNA 3'-end | | | | | |
| processing | | | | | |
| (GO: 0031441) | | | | | |
| viral gene expression | 22/110 | 1.96E−02 | 6.57E−01 | 1.73 | NUP214; NUP205; RPL3; RPS5; RPLPO; RPL13A; RPSA; NUP153; |
| (GO: 0019080) | | | | | RPL23A; RPL10A; AAAS; RPL6; RPL7; NUP93; RPS14; RPL7A; |
| | | | | | TPR; RPL14; RPS3; NUP98; RPL15; RPS23 |
| anion transmembrane | 6/18 | 1.99E−02 | 6.57E−01 | 3.44 | SLC13A1; SLC25A16; ABCC1; SLC13A4; LRRC8A; SLC25A42 |
| transport (GO: 0098656) | | | | | |
| negative regulation of | 6/18 | 1.99E−02 | 6.57E−01 | 3.44 | PRKN; VNN1; BAG5; NONO; HIF1A; NFE2L2 |
| oxidative stress-induced | | | | | |
| intrinsic apoptotic | | | | | |
| signaling pathway | | | | | |
| (GO: 1902176) | | | | | |
| release of cytochrome c | 6/18 | 1.99E−02 | 6.57E−01 | 3.44 | GGCT; BCL2A1; BAX; SFN; MFF; BCL2L1 |
| from mitochondria | | | | | |
| (GO: 0001836) | | | | | |
| negative regulation of | 8/28 | 1.99E−02 | 6.57E−01 | 2.76 | CD74; TMEM161A; DDIAS; RPS3; SNAI1; THOC5; CD44; BCL2L1 |
| response to DNA | | | | | |
| damage stimulus | | | | | |
| (GO: 2001021) | | | | | |
| protein K11-linked | 8/28 | 1.99E−02 | 6.57E−01 | 2.76 | PRKN; UBE2W; UBE2H; UBE2C; UBE2T; ANAPC4; RNF4; |
| ubiquitination | | | | | ANAPC10 |
| (GO: 0070979) | | | | | |
| carboxylic acid catabolic | 8/28 | 1.99E−02 | 6.57E−01 | 2.76 | ABHD10; NAGK; GNPDA1; HYAL1; HEXB; HMMR; CD44; ACOT4 |
| process (GO: 0046395) | | | | | |
| regulation of GTPase | 34/188 | 2.06E−02 | 6.74E−01 | 1.53 | GPSM1; CCL13; SNX13; ADRB1; F11R; RASGRP2; DOCK11; |
| activity (GO: 0043087) | | | | | SYDE1; RAP1A; WNT11; RACGAP1; SH3BP1; CCL7; LipidBB2; |
| | | | | | CCL2; ECT2; HRAS; S100A10; VAV3; ARFGEF1; CAV2; F2R; OCRL; |
| | | | | | ARHGAP27; VAV2; SOD1; SNX18; DNMBP; TBC1D7; RGS10; |
| | | | | | PLXNB2; PLXNB1; NGEF; CCL26 |
| regulation of extrinsic | 10/39 | 2.10E−02 | 6.83E−01 | 2.38 | SP100; RNF34; TRADD; FGG; TMBIM1; TNFSF10; TNFAIP3; |
| apoptotic signaling | | | | | PMAIP1; FAS; BCL2L1 |
| pathway via death | | | | | |
| domain receptors | | | | | |
| (GO: 1902041) | | | | | |
| negative regulation of | 14/62 | 2.15E−02 | 6.91E−01 | 2.01 | PRKN; NDUFA13; TMEM161A; BCL2A1; SRC; DDIAS; PLAUR; |
| intrinsic apoptotic | | | | | CREB3; BAG5; VNN1; CREB3L1; SNAI1; NFE2L2; BCL2L1 |
| signaling pathway | | | | | |
| (GO: 2001243) | | | | | |
| cholesterol metabolic | 15/68 | 2.17E−02 | 6.91E−01 | 1.95 | IDI1; FDPS; ACAA2; MVK; MSMO1; ACAT2; SULT2B1; ACLY; |
| process (GO: 0008203) | | | | | PLPP6; EBP; RXRA; PMVK; DHCR7; ABCG1; APOL2 |
| regulation of gene | 16/74 | 2.17E−02 | 6.91E−01 | 1.90 | SUZ12; DNMT1; KDM1B; GLMN; SMARCA5; CD3EAP; DDX21; |
| expression, epigenetic | | | | | BMI1; BAZ1B; KAT2B; IFI16; TAF1B; MYBBP1A; POLR1C; |
| (GO: 0040029) | | | | | ZNF335; SF3B1 |
| protein polymerization | 11/45 | 2.25E−02 | 7.05E−01 | 2.23 | PRKN; GPX4; UBE2C; CENPJ; FGG; MSRB2; ANG; CHMP4A; |
| (GO: 0051258) | | | | | MAPT; CKAP5; MSRB1 |
| regulation of alcohol | 9/34 | 2.28E−02 | 7.05E−01 | 2.48 | IDI1; FDPS; MVK; GPAM; PMVK; SC5D; SCAP; DHCR7; RAN |
| biosynthetic process | | | | | |
| (GO: 1902930) | | | | | |
| response to glucagon | 9/34 | 2.28E−02 | 7.05E−01 | 2.48 | GNG2; CREB1; ADCY9; PRKAR2B; CRY1; ADCY3; ADCY8; |
| (GO: 0033762) | | | | | PRKACA; ADCY5 |
| positive regulation of | 9/34 | 2.28E−02 | 7.05E−01 | 2.48 | SDCBP; SDC4; HGS; FGG; RAB27A; RAB27B; SYTL4; UNC13D; |
| exocytosis | | | | | CLASP2 |
| (GO: 0045921) | | | | | |
| CAMP biosynthetic | 5/14 | 2.45E−02 | 7.42E−01 | 3.83 | ADCY10; ADCY9; ADCY3; ADCY8; ADCY5 |
| process (GO: 0006171) | | | | | |
| acetyl-CoA metabolic | 5/14 | 2.45E−02 | 7.42E−01 | 3.83 | ACLY; NUDT7; MVK; PMVK; ACAT1 |
| process (GO: 0006084) | | | | | |
| regulation of mitotic | 33/184 | 2.49E−02 | 7.42E−01 | 1.51 | ANAPC16; DCTN2; CEP164; CUL1; KIF14; HMMR; ANAPC10; |
| cell cycle phase | | | | | AURKA; CDC20; CCNB1; TUBA1A; PSMB2; PRKAR2B; PSMD1; |
| transition (GO: 1901990) | | | | | KNTC1; NEK2; FBXO5; PRKACA; YWHAG; PLK4; UBE2C; ODF2; |
| | | | | | TUBB; PLK1; CDC6; CKAP5; SIRT2; TPX2; ANLN; PSMA3; CENPJ; |
| | | | | | PSME1; ANAPC4 |
| phosphatidic acid | 10/40 | 2.49E−02 | 7.42E−01 | 2.30 | GNPAT; GPAM; LPCAT4; DGKA; LIPH; GPD1; DDHD2; PNPLA3; |
| biosynthetic process | | | | | PLA2G5; GPAT2 |
| (GO: 0006654) | | | | | |
| regulation of viral | 10/40 | 2.49E−02 | 7.42E−01 | 2.30 | IFITM3; ZFP36; TRIM62; CCNT1; MDFIC; DHX9; NELFCD; |
| transcription | | | | | SUPT5H; TRIM21; MID2 |
| (GO: 0046782) | | | | | |
| regulation of | 23/119 | 2.53E−02 | 7.47E−01 | 1.65 | NUP214; BARD1; CDKN1C; NUP205; CDKN1B; STAT2; GLMN; |
| phosphorylation | | | | | IFNA2; NUP153; BCL10; AAAS; SIRT2; EGFR; PML; NUP93; IKBKB; |
| (GO: 0042325) | | | | | SDCBP; TPR; PLXNB2; NCKAP1L; ANG; NUP98; SLC25A33 |
| negative regulation of | 7/24 | 2.57E−02 | 7.48E−01 | 2.84 | CD74; TMEM161A; DDIAS; SNAI1; CD44; ZNF385A; BCL2L1 |
| intrinsic apoptotic | | | | | |
| signaling pathway in | | | | | |
| response to DNA | | | | | |
| damage (GO: 1902230) | | | | | |
| maturation of 5.8S | 6/19 | 2.60E−02 | 7.48E−01 | 3.18 | EXOSC7; PDCD11; RPSA; KRI1; WDR12; EXOSC2 |
| rRNA from tricistronic | | | | | |
| rRNA transcript (SSU- | | | | | |
| rRNA, 5.8S rRNA, LSU- | | | | | |
| rRNA) (GO: 0000466) | | | | | |
| regulation of | 6/19 | 2.60E−02 | 7.48E−01 | 3.18 | PRKN; INF2; STAT2; MAPT; MFF; DCN |
| mitochondrial fission | | | | | |
| (GO: 0090140) | | | | | |
| acylglycerol | 6/19 | 2.60E−02 | 7.48E−01 | 3.18 | GNPAT; GPAM; PNPLA3; ANG; GPAT2; LPIN1 |
| biosynthetic process | | | | | |
| (GO: 0046463) | | | | | |
| regulation of cyclin- | 9/35 | 2.74E−02 | 7.51E−01 | 2.39 | CDKN1C; BLM; CCNK; CDK7; CDKN1B; CCNT1; CDC6; CDC25C; |
| dependent protein | | | | | CDKN3 |
| kinase activity | | | | | |
| (GO: 1904029) | | | | | |
| maturation of SSU-rRNA | 9/35 | 2.74E−02 | 7.51E−01 | 2.39 | RPS14; PDCD11; UTP3; TSR1; RPSA; NOL10; KRI1; WDR46; |
| from tricistronic rRNA | | | | | DCAF13 |
| transcript (SSU-rRNA, | | | | | |
| 5.8S rRNA, LSU-rRNA) | | | | | |
| (GO: 0000462) | | | | | |
| tRNA transport | 9/35 | 2.74E−02 | 7.51E−01 | 2.39 | NUP214; NUP93; NUP205; TPR; NUP153; NUP98; AAAS; RAN; |
| (GO: 0051031) | | | | | NOL6 |
| tRNA modification | 13/58 | 2.77E−02 | 7.51E−01 | 1.99 | TRUB1; TRMT10A; PUS10; OSGEPL1; METTL1; THUMPD2; |
| (GO: 0006400) | | | | | TRMT1; THADA; MTO1; FTSJ1; C9ORF64; THG1L; TRMT61A |
| nuclear pore complex | 4/10 | 2.89E−02 | 7.51E−01 | 4.59 | NUP93; NUP205; NUP153; NUP98 |
| assembly (GO: 0051292) | | | | | |
| negative regulation of | 4/10 | 2.89E−02 | 7.51E−01 | 4.59 | ACVR1C; DUSP1; TIMP1; NODAL |
| reproductive process | | | | | |
| (GO: 2000242) | | | | | |
| mitochondrial | 4/10 | 2.89E−02 | 7.51E−01 | 4.59 | UQCC1; LYRM7; UQCR10; SLC25A33 |
| respiratory chain | | | | | |
| complex III biogenesis | | | | | |
| (GO: 0097033) | | | | | |
| positive regulation of | 4/10 | 2.89E−02 | 7.51E−01 | 4.59 | FAP; RPS3; BAX; SIRT2 |
| execution phase of | | | | | |
| apoptosis | | | | | |
| (GO: 1900119) | | | | | |
| regulation of cellular | 10/41 | 2.93E−02 | 7.51E−01 | 2.22 | PRKN; ACLY; RNF34; NPC2; GBA; CYP1B1; FOXM1; DKK1; |
| metabolic process | | | | | ABCG1; TBC1D16 |
| (GO: 0031323) | | | | | |
| phosphatidic acid | 10/41 | 2.93E−02 | 7.51E−01 | 2.22 | GNPAT; GPAM; LPCAT4; DGKA; LIPH; GPD1; DDHD2; PNPLA3; |
| metabolic process | | | | | PLA2G5; GPAT2 |
| (GO: 0046473) | | | | | |
| positive regulation of | 38/220 | 2.94E−02 | 7.51E−01 | 1.44 | FAM49B; PRKDC; DHX9; HILPDA; HTR2B; PTPN22; DDX60; |
| cytokine production | | | | | HIF1A; RELA; DHX33; IRAK1; IFI16; RPS3; CYP1B1; STAT6; IL6R; |
| (GO: 0001819) | | | | | HLA-DPA1; GBP5; CREBBP; XRCC5; ADIPOQ; GLMN; CYBA; |
| | | | | | TICAM1; PUM2; SOD1; FGR; IL6; POLR3C; IRF1; POLR1C; |
| | | | | | TLR9; PRKD2; POLR3H; HPSE; CD46; NODAL; MYD88 |
| regulation of | 8/30 | 2.98E−02 | 7.51E−01 | 2.51 | GHR; STAT5B; OLFM1; CTTN; INSR; SIX1; FGFR2; SOD1 |
| developmental growth | | | | | |
| (GO: 0048638) | | | | | |
| protein repair | 3/6 | 3.04E−02 | 7.51E−01 | 6.88 | MSRA; MSRB2; MSRB1 |
| (GO: 0030091) | | | | | |
| regulation of oxidative | 3/6 | 3.04E−02 | 7.51E−01 | 6.88 | PRKN; NONO; HIF1A |
| stress-induced neuron | | | | | |
| intrinsic apoptotic | | | | | |
| signaling pathway | | | | | |
| (GO: 1903376) | | | | | |
| establishment of | 3/6 | 3.04E−02 | 7.51E−01 | 6.88 | TJP1; F11R; TJP2 |
| endothelial intestinal | | | | | |
| barrier (GO: 0090557) | | | | | |
| choline catabolic | 3/6 | 3.04E−02 | 7.51E−01 | 6.88 | DMGDH; SLC44A1; SARDH |
| process (GO: 0042426) | | | | | |
| citrate metabolic | 3/6 | 3.04E−02 | 7.51E−01 | 6.88 | GHR; STAT5B; ACLY |
| process (GO: 0006101) | | | | | |
| signal complex | 3/6 | 3.04E−02 | 7.51E−01 | 6.88 | SRC; PXN; NCK2 |
| assembly (GO: 0007172) | | | | | |
| protein insertion into | 3/6 | 3.04E−02 | 7.51E−01 | 6.88 | BCL2L11; BAX; PMAIP1 |
| mitochondrial | | | | | |
| membrane involved in | | | | | |
| apoptotic signaling | | | | | |
| pathway (GO: 0001844) | | | | | |
| tRNA catabolic process | 3/6 | 3.04E−02 | 7.51E−01 | 6.88 | EXOSC7; POP1; EXOSC2 |
| (GO: 0016078) | | | | | |
| establishment of | 3/6 | 3.04E−02 | 7.51E−01 | 6.88 | ACD; POT1; WRAP53 |
| protein localization to | | | | | |
| telomere (GO: 0070200) | | | | | |
| granzyme-mediated | 3/6 | 3.04E−02 | 7.51E−01 | 6.88 | SRGN; LAMP1; GZMB |
| apoptotic signaling | | | | | |
| pathway (GO: 0008626) | | | | | |
| transmembrane | 3/6 | 3.04E−02 | 7.51E−01 | 6.88 | TRIO; FRS2; PTPRF |
| receptor protein | | | | | |
| tyrosine phosphatase | | | | | |
| signaling pathway | | | | | |
| (GO: 0007185) | | | | | |
| response to insulin | 15/71 | 3.12E−02 | 7.51E−01 | 1.85 | PRKDC; SORT1; INSR; PDE3B; SYAP1; ADIPOQ; DENND4C; |
| (GO: 0032868) | | | | | PCSK9; SORBS1; KAT2B; TBC1D4; SLC25A33; RAB8A; RHOQ; |
| | | | | | VAMP2 |
| peptidyl-lysine | 22/115 | 3.13E−02 | 7.51E−01 | 1.63 | NUP214; TOP2A; NUP205; L3MBTL2; UBE21; PCNA; BCL11A; |
| modification | | | | | NUP153; P3H3; AAAS; PIAS2; SIRT2; SENP1; NUP93; KAT2B; |
| (GO: 0018205) | | | | | SUMO1; INCENP; TPR; BIRC5; ETFBKMT; NUP98; IVL |
| cellular response to | 14/65 | 3.15E−02 | 7.51E−01 | 1.89 | PXN; TNFAIP3; RELA; EGFR; PRDX3; MAPK9; PRDX5; IL6; |
| reactive oxygen species | | | | | PXMP2; RPS3; CYP1B1; MAPT; ECT2; NFE2L2 |
| (GO: 0034614) | | | | | |
| positive regulation of | 19/96 | 3.16E−02 | 7.51E−01 | 1.70 | PRKN; BARD1; RAB1A; NDUFA13; CDKN1B; EGLN2; CSF1; |
| protein metabolic | | | | | SMURF2; PRMT1; TIPARP; PLK2; ADIPOQ; GBA; TNFAIP3; |
| process (GO: 0051247) | | | | | NR1H3; CNPY2; KLF4; OAZ3; SCAP |
| response to alcohol | 7/25 | 3.19E−02 | 7.51E−01 | 2.68 | SMAD2; ABCA2; GRIN2A; GPRIN1; PMVK; SDF4; SOD1 |
| (GO: 0097305) | | | | | |
| cellular response to | 7/25 | 3.19E−02 | 7.51E−01 | 2.68 | STAT5B; ZFP36; GAREM1; SYAP1; LipidBB2; EGFR; ZPR1 |
| epidermal growth | | | | | |
| factor stimulus | | | | | |
| (GO: 0071364) | | | | | |
| endoderm formation | 9/36 | 3.25E−02 | 7.51E−01 | 2.30 | DUSP5; COL4A2; DUSP1; HMGA2; ITGA7; ITGA5; MMP8; |
| (GO: 0001706) | | | | | NODAL; MIXL1 |
| regulation of ubiquitin | 9/36 | 3.25E−02 | 7.51E−01 | 2.30 | CDC20; RAB1A; CCNB1; ANAPC16; UBE2C; PLK1; ANAPC4; |
| protein ligase activity | | | | | FBXO5; ANAPC10 |
| (GO: 1904666) | | | | | |
| fatty acid beta- | 5/15 | 3.29E−02 | 7.51E−01 | 3.44 | SCP2; ACOX1; EHHADH; ECI2; HSD17B4 |
| oxidation using acyl- | | | | | |
| CoA oxidase | | | | | |
| (GO: 0033540) | | | | | |
| nuclear pore | 5/15 | 3.29E−02 | 7.51E−01 | 3.44 | NUP93; NUP205; TPR; NUP153; NUP98 |
| organization | | | | | |
| (GO: 0006999) | | | | | |
| regulation of mitotic | 5/15 | 3.29E−02 | 7.51E−01 | 3.44 | ESPL1; UBE2C; PLK1; CDC6; DLGAP5 |
| metaphase/anaphase | | | | | |
| transition (GO: 0030071) | | | | | |
| regulation of centriole | 5/15 | 3.29E−02 | 7.51E−01 | 3.44 | PLK4; KAT2B; STIL; PLK2; CENPJ |
| replication | | | | | |
| (GO: 0046599) | | | | | |
| fatty acid beta- | 5/15 | 3.29E−02 | 7.51E−01 | 3.44 | GCDH; ACOX1; IVD; ETFA; ACAD10 |
| oxidation using acyl- | | | | | |
| CoA dehydrogenase | | | | | |
| (GO: 0033539) | | | | | |
| negative regulation of | 5/15 | 3.29E−02 | 7.51E−01 | 3.44 | PRKN; CD74; RNF34; SNAI1; CD44 |
| signal transduction by | | | | | |
| p53 class mediator | | | | | |
| (GO: 1901797) | | | | | |
| positive regulation of | 5/15 | 3.29E−02 | 7.51E−01 | 3.44 | JUP; TPR; ECT2; RBM22; ZPR1 |
| protein import | | | | | |
| (GO: 1904591) | | | | | |
| hydrogen peroxide | 6/20 | 3.33E−02 | 7.51E−01 | 2.95 | PRDX3; PRDX2; PRDX5; CAT; CYBA; SOD1 |
| metabolic process | | | | | |
| (GO: 0042743) | | | | | |
| purine nucleotide | 6/20 | 3.33E−02 | 7.51E−01 | 2.95 | NT5E; MTHFD1; NT5C1A; NUDT1; FHIT; DNPH1 |
| metabolic process | | | | | |
| (GO: 0006163) | | | | | |
| regulation of | 10/42 | 3.43E−02 | 7.69E−01 | 2.15 | PIF1; ACD; PARP4; SRC; XRCC5; POT1; NEK2; MAPKAPK5; KLF4; |
| telomerase activity | | | | | WRAP53 |
| (GO: 0051972) | | | | | |
| mitochondrial | 19/97 | 3.48E−02 | 7.71E−01 | 1.68 | NDUFA13; COA1; NDUFA6; NDUFA5; NDUFB5; UQCC1; |
| respiratory chain | | | | | NDUFB4; COX17; NDUFB3; NDUFA2; UQCR10; PET117; SCO1; |
| complex assembly | | | | | LYRM7; NDUFAF3; COX14; SDHAF4; NDUFAF1; SLC25A33 |
| (GO: 0033108) | | | | | |
| pattern recognition | 11/48 | 3.52E−02 | 7.71E−01 | 2.05 | IRAK1; CTSL; S100A1; FGG; TLR9; LY96; BCL10; CD36; CNPY3; |
| receptor signaling | | | | | MYD88; CTSB |
| pathway (GO: 0002221) | | | | | |
| cellular response to | 21/110 | 3.55E−02 | 7.71E−01 | 1.63 | ATP6VOB; PRKDC; CAV2; INSR; SYAP1; ADIPOQ; DENND4C; |
| insulin stimulus | | | | | PDE3B; PCSK9; SORBS1; KAT2B; TBC1D4; SOS1; ATP6V1G3; |
| (GO: 0032869) | | | | | PAT6V1E2; ATP6V1D; SLC25A33; RAB8A; VAMP2; RHOQ; |
| | | | | | ATP6V1C2 |
| intracellular transport | 12/54 | 3.57E−02 | 7.71E−01 | 1.97 | NUP214; NUP93; NUP205; TPR; KPNA7; UBAP1; NUP153; |
| of virus (GO: 0075733) | | | | | KPNA5; NUP98; KPNA2; AAAS; RAN |
| cytoplasmic translation | 12/54 | 3.57E−02 | 7.71E−01 | 1.97 | MRPL2; RPLPO; RPS3; RPL22L1; RPL15; RPL10A; HSPA14; FTSJ1; |
| (GO: 0002181) | | | | | RPL6; EIF4B; RPL7; RPS23 |
| transport of virus | 12/54 | 3.57E−02 | 7.71E−01 | 1.97 | NUP214; NUP93; NUP205; TPR; KPNA7; UBAP1; NUP153; KPNA5; |
| (GO: 0046794) | | | | | NUP98; KPNA2; AAAS; RAN |
| positive regulation of | 8/31 | 3.58E−02 | 7.71E−01 | 2.40 | CDC20; RAB1A; DCUN1D5; UBE2C; PLK1; FBXO5; BMI1; |
| ubiquitin-protein | | | | | ANAPC10 |
| transferase activity | | | | | |
| (GO: 0051443) | | | | | |
| negative regulation of | 8/31 | 3.58E−02 | 7.71E−01 | 2.40 | RNF34; TRADD; FGG; TMBIM1; TNFSF10; TNFAIP3; FAS; |
| extrinsic apoptotic | | | | | BCL2L1 |
| signaling pathway via | | | | | |
| death domain receptors | | | | | |
| (GO: 1902042) | | | | | |
| protein | 9/37 | 3.83E−02 | 8.16E−01 | 2.21 | PRKN; UBE2W; DTX3L; UBE2R2; UBE2T; CTR9; TRIM25; |
| monoubiquitination | | | | | TRIM21; CDC73 |
| (GO: 0006513) | | | | | |
| regulation of DNA | 9/37 | 3.83E−02 | 8.16E−01 | 2.21 | BABAM2; PCNA; TMEM161A; DHX9; ABRAXAS1; UIMC1; RPS3; |
| repair (GO: 0006282) | | | | | EGFR; PML |
| cytoskeleton- | 7/26 | 3.90E−02 | 8.27E−01 | 2.54 | TUBA1C; TUBA1A; HOOK2; TUBB; KIF13A; KIF14; DYNLL2 |
| dependent intracellular | | | | | |
| transport (GO: 0030705) | | | | | |
| regulation of cyclin- | 14/67 | 3.97E−02 | 8.34E−01 | 1.82 | HEXIM1; CCNK; BLM; CDKN1B; CCNT1; CCNH; PLK1; CDC6; |
| dependent protein | | | | | CDC25C; CKS1B; CDK7; CKS2; CCNL2; CDKN3 |
| serine/threonine kinase | | | | | |
| activity (GO: 0000079) | | | | | |
| response to hydrogen | 10/43 | 3.97E−02 | 8.34E−01 | 2.09 | PRDX3; IL6; CAT; RPS3; TNFAIP3; CYP1B1; ECT2; RELA; SOD1; |
| peroxide (GO: 0042542) | | | | | NFE2L2 |
| positive regulation of | 12/55 | 4.05E−02 | 8.34E−01 | 1.92 | CYFIP2; NDUFA13; GRINZA; ACVR1C; ASPH; IFI16; GPRIN1; |
| cysteine-type | | | | | F2R; TNFSF10; PMAIP1; BLID; NODAL |
| endopeptidase activity | | | | | |
| (GO: 2001056) | | | | | |
| negative regulation of | 4/11 | 4.09E−02 | 8.34E−01 | 3.93 | BLM; SUSD2; E2F7; E2F8 |
| cell division | | | | | |
| (GO: 0051782) | | | | | |
| methionine metabolic | 4/11 | 4.09E−02 | 8.34E−01 | 3.93 | MSRA; AHCYL1; AHCY; MTHFD1 |
| process (GO: 0006555) | | | | | |
| mitotic spindle | 4/11 | 4.09E−02 | 8.34E−01 | 3.93 | RACGAP1; PRC1; KIF4A; KIF23 |
| elongation | | | | | |
| (GO: 0000022) | | | | | |
| regulation of mRNA | 4/11 | 4.09E−02 | 8.34E−01 | 3.93 | CCNT1; CTR9; SUPT5H; CPEB3 |
| polyadenylation | | | | | |
| (GO: 1900363) | | | | | |
| positive regulation of | 4/11 | 4.09E−02 | 8.34E−01 | 3.93 | NR1H3; SCAP; CNPY2; HIF1A |
| receptor biosynthetic | | | | | |
| process (GO: 0010870) | | | | | |
| positive regulation of | 74/479 | 4.16E−02 | 8.34E−01 | 1.26 | KLB; GPR89A; ECM1; CXCL9; SLC44A2; TRADD; HTR2B; PIK3CB; |
| intracellular signal | | | | | EDA2R; IKBKB; FGF5; TNFSF10; TRIM25; HRAS; TRIM22; TRIM62; |
| transduction | | | | | F2R; ADIPOQ; FRS2; SOX11; TICAM1; ADRA2C; TNFRSB; F1 |
| (GO: 1902533) | | | | | WNT16; MID2; DCN; FGR; TYRO3; TLR9; HCLS1; PRKD2; PRKD1; |
| | | | | | PRKN; BECN1; SRC; SLC20A1; ADRB1; BCL10; DDX60; ATP2C1; |
| | | | | | EGFR; RELA; BCL2L11; WNT11; DHX33; THPO; IRAK1; C19; D |
| | | | | | LipidBB2; RPS3; PMAIP1; ECT2; APOL3; HEXIM1; KL; MAP3K3; |
| | | | | | CD74; NDFIP1; CAV2; PLK2; INSR; SIAH1; LIF; PUM2; IL6; TNIP2; |
| | | | | | FAS; BAX; PLXNB1; HPSE; LTBR; MYD88; FGFR2; BCL2L1 |
| phospholipid | 6/21 | 4.17E−02 | 8.34E−01 | 2.75 | ABCC1; ATP8B2; ATP10B; ATP11B; ATP11A; ATP9A |
| translocation | | | | | |
| (GO: 0045332) | | | | | |
| mitochondrial | 8/32 | 4.26E−02 | 8.34E−01 | 2.30 | NDUFA13; SMDT1; TIMM29; UCP2; TIMM22; THRSP; SLC25A33; |
| transmembrane | | | | | SLC25A36 |
| transport (GO: 1990542) | | | | | |
| regulation of SMAD | 5/16 | 4.30E−02 | 8.34E−01 | 3.13 | NUP93; DAB2; SMAD3; NODAL; PBLD |
| protein import into | | | | | |
| nucleus (GO: 0060390) | | | | | |
| linoleic acid metabolic | 5/16 | 4.30E−02 | 8.34E−01 | 3.13 | CYP2J2; FADS2; ELOVL5; PNPLA8; ACSL1 |
| process (GO: 0043651) | | | | | |
| positive regulation of | 5/16 | 4.30E−02 | 8.34E−01 | 3.13 | PPP1R15A; ATXN3; BCL2L11; BAX; PMAIP1 |
| response to | | | | | |
| endoplasmic reticulum | | | | | |
| stress (GO: 1905898) | | | | | |
| regulation of intrinsic | 5/16 | 4.30E−02 | 8.34E−01 | 3.13 | TMEM161A; DDIAS; SNAI1; RPS3; BCL2L1 |
| apoptotic signaling | | | | | |
| pathway in response to | | | | | |
| DNA damage | | | | | |
| (GO: 1902229) | | | | | |
| regulation of reactive | 5/16 | 4.30E−02 | 8.34E−01 | 3.13 | GPRIN1; RAB27A; CYBA; CD36; SLC25A33 |
| oxygen species | | | | | |
| biosynthetic process | | | | | |
| (GO: 1903426) | | | | | |
| negative regulation of | 5/16 | 4.30E−02 | 8.34E−01 | 3.13 | DKKL1; SNAI1; SCAP; LipidLIN2; SOD1 |
| steroid biosynthetic | | | | | |
| process (GO: 0010894) | | | | | |
| regulation of histone | 5/16 | 4.30E−02 | 8.34E−01 | 3.13 | DNMT1; AUTS2; KMT2A; CTR9; WDR61 |
| H3-K4 methylation | | | | | |
| (GO: 0051569) | | | | | |
| positive regulation of | 30/172 | 4.33E−02 | 8.34E−01 | 1.46 | CCL13; NDUFA13; MTCL1; SNX13; ADRB1; F11R; RASGRP2; |
| hydrolase activity | | | | | DOCK11; RAP1A; WNT11; SH3BP1; CCL7; LipidBB2; RPS3; CCL2; |
| (GO: 0051345) | | | | | ECT2; HRAS; S100A10; CAV2; F2R; ARHGAP27; MSH6; SNX18; |
| | | | | | DNMBP; MYL3; TBC1D7; RGS10; POT1; PLXNB1; CCL26 |
| mitotic nuclear division | 15/74 | 4.34E−02 | 8.34E−01 | 1.75 | ARHGEF10; SPAG5; UBE2C; PLK1; NSL1; HIRA; TPX2; KIF18B; |
| (GO: 0140014) | | | | | ESPL1; CHEK2; PPP2R2D; NUSAP1; BIRC5; RAN; CLASP2 |
| positive regulation of | 20/106 | 4.40E−02 | 8.39E−01 | 1.60 | SH3GLB1; PFKFB2; ABCD2; DTX3L; INSR; PLK2; TFEB; ZC3HAV1; |
| cellular catabolic | | | | | PIK3CB; SNX33; TICAM1; TNFRSF1B; HIF1A; SVIP; MID2; DCN; |
| process (GO: 0031331) | | | | | KHSRP; PRKD1; TRIM21; TRIM22 |
| regulation of translation | 36/213 | 4.40E−02 | 8.39E−01 | 1.40 | CYFIP2; NUP205; CDC123; DHX9; EIF4A3; RBM4B; MALSU1; |
| (GO: 0006417) | | | | | AAAS; TYMS; RIDA; TPR; LipidBB2; RPS3; ZNF385A; EIF4B; |
| | | | | | NUP214; EIF2B4; EIF5B; EIF2B2; SAMD4A; SAMD4B; RPL13A; |
| | | | | | UPF3A; NUP153; LARP4; PUM2; NUP93; DHFR; ILF3; IL6; EIF5; |
| | | | | | MAPKAPK5; NUP98; CPEB3; RAN; EIF3B |
| positive regulation of | 9/38 | 4.47E−02 | 8.49E−01 | 2.14 | BABAM2; PCNA; TMEM161A; DHX9; ABRAXAS1; UIMC1; RPS3; |
| DNA repair | | | | | FOXM1; EGFR |
| (GO: 0045739) | | | | | |
| mitotic nuclear | 10/44 | 4.58E−02 | 8.53E−01 | 2.03 | NUP214; NUP93; NUP205; CCNB1; TPR; PLK1; NUP153; NUP98; |
| envelope disassembly | | | | | AAAS; LPIN1 |
| (GO: 0007077) | | | | | |
| ribosome assembly | 12/56 | 4.59E−02 | 8.53E−01 | 1.88 | RPS14; RPL3; RPS5; EFL1; NOP2; MPV17L2; RPSA; BRIX1; |
| (GO: 0042255) | | | | | RPL23A; RPL6; MDN1; NLE1 |
| intracellular sterol | 3/7 | 4.82E−02 | 8.53E−01 | 5.16 | STARD4; NPC2; ABCG1 |
| transport (GO: 0032366) | | | | | |
| modulation by | 3/7 | 4.82E−02 | 8.53E−01 | 5.16 | KPNA7; KPNA5; KPNA2 |
| symbiont of host | | | | | |
| cellular process | | | | | |
| (GO: 0044068) | | | | | |
| regulation of DNA | 3/7 | 4.82E−02 | 8.53E−01 | 5.16 | SMC3; E2F7; E2F8 |
| endoreduplication | | | | | |
| (GO: 0032875) | | | | | |
| pri-miRNA transcription | 3/7 | 4.82E−02 | 8.53E−01 | 5.16 | FOSL1; KLF4; RELA |
| from RNA polymerase II | | | | | |
| promoter (GO: 0061614) | | | | | |
| protein import into | 3/7 | 4.82E−02 | 8.53E−01 | 5.16 | NDUFA13; TIMM29; TIMM22 |
| mitochondrial inner | | | | | |
| membrane | | | | | |
| (GO: 0045039) | | | | | |
| L-cysteine metabolic | 3/7 | 4.82E−02 | 8.53E−01 | 5.16 | AHCYL1; AHCY; CBS |
| process (GO: 0046439) | | | | | |
| regulation of low- | 3/7 | 4.82E−02 | 8.53E−01 | 5.16 | ADIPOQ; SCAP; CNPY2 |
| density lipoprotein | | | | | |
| particle receptor | | | | | |
| biosynthetic process | | | | | |
| (GO: 0045714) | | | | | |
| cellular lipid | 3/7 | 4.82E−02 | 8.53E−01 | 5.16 | GNPAT; AGPS; ACAT1 |
| biosynthetic process | | | | | |
| (GO: 0097384) | | | | | |
| negative regulation of | 3/7 | 4.82E−02 | 8.53E−01 | 5.16 | FRS2; DKK1; EGFR |
| cardiocyte | | | | | |
| differentiation | | | | | |
| (GO: 1905208) | | | | | |
| G-quadruplex DNA | 3/7 | 4.82E−02 | 8.53E−01 | 5.16 | PIF1; BLM; DHX9 |
| unwinding | | | | | |
| (GO: 0044806) | | | | | |
| long-chain fatty acid | 3/7 | 4.82E−02 | 8.53E−01 | 5.16 | ACSL1; ACSL3; CD36 |
| import (GO: 0044539) | | | | | |
| leucine metabolic | 3/7 | 4.82E−02 | 8.53E−01 | 5.16 | MCCC2; HMGCL; IVD |
| process (GO: 0006551) | | | | | |
| rRNA-containing | 3/7 | 4.82E−02 | 8.53E−01 | 5.16 | RPSA; ABCE1; RAN |
| ribonucleoprotein | | | | | |
| complex export from | | | | | |
| nucleus (GO: 0071428) | | | | | |
| positive regulation of | 3/7 | 4.82E−02 | 8.53E−01 | 5.16 | FGR; SNX4; VAMP7 |
| mast cell degranulation | | | | | |
| (GO: 0043306) | | | | | |
| mitochondrion | 29/167 | 4.86E−02 | 8.57E−01 | 1.45 | PRKN; PIF1; BCL2A1; MFF; HSD17B10; HK2; IFIT2; PRDX3; |
| organization | | | | | RHOT1; TSPO; USP30; ESRRA; BNIP3L; AGTPBP1; CAV2; MX2; |
| (GO: 0007005) | | | | | MTOM34; TIMM22; PPRC1; DNM1; ATAD3C; HCFC1; TMEM11; |
| | | | | | YME1L1; BAX; SLC25A33; SLC25A36; BCL2L1; ATPAF1 |
| regulation of | 8/33 | 5.02E−02 | 8.82E−01 | 2.20 | PLK3; RIPOR1; IFI27; TPR; XPO5; ADIPOQ; VAMP2; UHMK1 |
| intracellular protein | | | | | |
| transport (GO: 0033157) | | | | | |
| positive regulation of | 16/82 | 5.13E−02 | 8.88E−01 | 1.67 | CCNK; CDKN1B; DUSP3; CCNT1; CCNH; CKS1B; CDC25B; |
| cell cycle (GO: 0045787) | | | | | HCFC1; CCNB1; CHEK1; CKS2; PKN2; CCNL2; TRIM21; |
| | | | | | ZNF268; FGFR2 |
| regulation of | 6/22 | 5.14E−02 | 8.88E−01 | 2.58 | PRDX5; BRF2; ICE2; POLR3C; NAB2; LipidBB2 |
| transcription from RNA | | | | | |
| polymerase Ill promoter | | | | | |
| (GO: 0006359) | | | | | |
| positive regulation of | 6/22 | 5.14E−02 | 8.88E−01 | 2.58 | CREB1; GPRIN1; RAB27A; CYBA; SORBS1; CD36 |
| biosynthetic process | | | | | |
| (GO: 0009891) | | | | | |
| lipid translocation | 6/22 | 5.14E−02 | 8.88E−01 | 2.58 | ABCC1; ATP8B2; ATP10B; ATP11B; ATP11A; ATP9A |
| (GO: 0034204) | | | | | |
| positive regulation of | 9/39 | 5.19E−02 | 8.92E−01 | 2.07 | ZFP36; CREB1; FRZB; SYAP1; ADIRF; METRNL; AAMDC; |
| fat cell differentiation | | | | | ZNF385A; MEDAG |
| (GO: 0045600) | | | | | |
| positive regulation of | 15/76 | 5.31E−02 | 8.97E−01 | 1.70 | IFNB1; SIAH1; PLAUR; GOS2; BCL10; MFF; PML; BCL2L11; |
| apoptotic signaling | | | | | TNFSF10; RPS3; PMAIP1; FAS; BAX; LTBR; BCL2L1 |
| pathway (GO: 2001235) | | | | | |
| protein complex | 32/189 | 5.38E−02 | 8.97E−01 | 1.41 | DPAGT1; RAB1A; BLM; CDC123; KHDRBS3; TRRAP; TRADD; |
| assembly (GO: 0006461) | | | | | PQRT; TNFAIP3; BCL10; CDC73; PPP6C; SCFD1; IRAK1; RBBP5; |
| | | | | | ZNHIT6; RUVBL1; PPP6R3; CD59; SEC31A; SH3GLB1; TLE3; |
| | | | | | TLE2; CREBBP; SEC16A; TRAPPC9; TRAPPC6A; NBR1; BAX; |
| | | | | | PREB; MAPT; BET1 |
| regulation of | 5/17 | 5.48E−02 | 8.97E−01 | 2.87 | GHR; STAT5B; PPIB; FGFR2; SOD1 |
| multicellular organism | | | | | |
| growth (GO: 0040014) | | | | | |
| response to interferon- | 5/17 | 5.48E−02 | 8.97E−01 | 2.87 | IFITM3; PYHIN1; MX2; TPR; ADAR |
| alpha (GO: 0035455) | | | | | |
| regulation of | 5/17 | 5.48E−02 | 8.97E−01 | 2.87 | RNF10; TENM4; KIF14; SIRT2; ZPR1 |
| myelination | | | | | |
| (GO: 0031641) | | | | | |
| peptidyl-lysine | 5/17 | 5.48E−02 | 8.97E−01 | 2.87 | KMT2A; CTR9; ETFBKMT; WDR61; ZNF335 |
| trimethylation | | | | | |
| (GO: 0018023) | | | | | |
| nucleotide catabolic | 5/17 | 5.48E−02 | 8.97E−01 | 2.87 | NT5E; NT5C1A; NUDT1; SAMHD1; DNPH1 |
| process (GO: 0009166) | | | | | |
| serine family amino | 5/17 | 5.48E−02 | 8.97E−01 | 2.87 | SEPSECS; SRR; MTHFD1; CBS; GLYAT |
| acid metabolic process | | | | | |
| (GO: 0009069) | | | | | |
| vascular endothelial | 14/70 | 5.49E−02 | 8.97E−01 | 1.72 | CYFIP2; VAV3; NCF2; SRC; PXN; CYBA; PIK3CB; PGF; VAV2; |
| growth factor receptor | | | | | ABI1; NCK2; PRKD2; NCKAP1L; PRKD1 |
| signaling pathway | | | | | |
| (GO: 0048010) | | | | | |
| activation of cysteine- | 4/12 | 5.54E−02 | 8.97E−01 | 3.44 | CYFIP2; GRIN2A; IFI16; ASPH |
| type endopeptidase | | | | | |
| activity (GO: 0097202) | | | | | |
| collagen metabolic | 4/12 | 5.54E−02 | 8.97E−01 | 3.44 | TNXB; P3H3; TRAM2; HIF1A |
| process (GO: 0032963) | | | | | |
| positive regulation of | 4/12 | 5.54E−02 | 8.97E−01 | 3.44 | CDT1; CDC7; E2F7; E2F8 |
| DNA-dependent DNA | | | | | |
| replication | | | | | |
| (GO: 2000105) | | | | | |
| negative regulation of | 4/12 | 5.54E−02 | 8.97E−01 | 3.44 | HEYL; DAB2; PIAS2; NODAL |
| androgen receptor | | | | | |
| signaling pathway | | | | | |
| (GO: 0060766) | | | | | |
| positive regulation of | 4/12 | 5.54E−02 | 8.97E−01 | 3.44 | NUP93; DAB2; SMAD3; NODAL |
| SMAD protein import | | | | | |
| into nucleus | | | | | |
| (GO: 0060391) | | | | | |
| Golgi localization | 4/12 | 5.54E−02 | 8.97E−01 | 3.44 | UVRAG; RIPOR1; TBCCD1; COPG1 |
| (GO: 0051645) | | | | | |
| positive regulation of | 7/28 | 5.62E−02 | 9.00E−01 | 2.30 | PPP2R1B; TNFSF10; PMAIP1; GOS2; BCL10; LTBR; PML |
| extrinsic apoptotic | | | | | |
| signaling pathway | | | | | |
| (GO: 2001238) | | | | | |
| cellular response to | 7/28 | 5.62E−02 | 9.00E−01 | 2.30 | IL6; RPS3; TNFAIP3; CYP1B1; ECT2; RELA; NFE2L2 |
| hydrogen peroxide | | | | | |
| (GO: 0070301) | | | | | |
| positive regulation of | 7/28 | 5.62E−02 | 9.00E−01 | 2.30 | SDCBP; IFNA2; GLMN; NCKAP1L; ANG; BCL10; EGFR |
| phosphate metabolic | | | | | |
| process (GO: 0045937) | | | | | |
| regulation of I-kappaB | 34/204 | 5.79E−02 | 9.25E−01 | 1.38 | PRKN; ECM1; GPR89A; SLC44A2; SLC20A1; TRADD; HTR2B; |
| kinase/NF-kappaB | | | | | TNFAIP3; BCL10; ATP2C1; EDA2R; RELA; IKBKB; IRAK1; |
| signaling (GO: 0043122) | | | | | TNFSF10; TRIM25; ECT2; TRIM22; APOL3; MAP3K3; CD74; |
| | | | | | TRIM62;NDFIP1; PLK2; ADIPOQ; F2R; TICAM1; MID2; PPM1B; |
| | | | | | TNIP2; TLR9; PRKD1; LTBR; MYD88 |
| apoptotic mitochondrial | 8/34 | 5.87E−02 | 9.26E−01 | 2.12 | GGCT; BCL2A1; BAX; SFN; MFF; HK2; IFIT2; BCL2L1 |
| changes (GO: 0008637) | | | | | |
| negative regulation of T | 8/34 | 5.87E−02 | 9.26E−01 | 2.12 | DUSP3; SDC4; IFNB1; IFNA2; GLMN; PTPN22; PDCD1LG2; |
| cell activation | | | | | PAG1 |
| (GO: 0050868) | | | | | |
| endoplasmic reticulum | 8/34 | 5.87E−02 | 9.26E−01 | 2.12 | RTN3; CAV2; VAPB; ATL1; LMAN2; SEC16A; TRAM2; SEC31A |
| organization | | | | | |
| (GO: 0007029) | | | | | |
| regulation of | 9/40 | 5.97E−02 | 9.34E−01 | 2.00 | PRKN; NUP214; SCFD1; LAMP1; SPAG5; MX2; AAAS; CRYAB; |
| intracellular transport | | | | | UNC13D |
| (GO: 0032386) | | | | | |
| nuclear envelope | 10/46 | 5.97E−02 | 9.34E−01 | 1.91 | NUP214; NUP93; NUP205; CCNB1; TPR; PLK1; NUP153; NUP98; |
| disassembly | | | | | AAAS; LPIN1 |
| (GO: 0051081) | | | | | |
| protein targeting to | 18/97 | 6.18E−02 | 9.38E−01 | 1.57 | RPL3; RPS5; RPLPO; RPL13A; RPSA; RPL23A; RPL10A; RPL6; |
| Lipid (GO: 0045047) | | | | | RPL7; RPS14; RPL7A; SPCS2; RPL14; RPS3; CHMP4A; RPL15; |
| | | | | | SEC62; RPS23 |
| regulation of | 6/23 | 6.24E−02 | 9.38E−01 | 2.43 | PHLDB1; STMN1; MAPT; SKA3; AURKA; CLASP2 |
| microtubule | | | | | |
| cytoskeleton | | | | | |
| organization | | | | | |
| (GO: 0070507) | | | | | |
| negative regulation of | 6/23 | 6.24E−02 | 9.38E−01 | 2.43 | IFITM3; TRIM62; ZFP36; HMGA2; TRIM21; MID2 |
| viral transcription | | | | | |
| (GO: 0032897) | | | | | |
| microtubule | 6/23 | 6.24E−02 | 9.38E−01 | 2.43 | KIF18B; CENPJ; STMN1; KIF24; MAPT; CKAP5 |
| polymerization or | | | | | |
| depolymerization | | | | | |
| (GO: 0031109) | | | | | |
| viral process | 36/220 | 6.52E−02 | 9.38E−01 | 1.35 | RAB1A; NUP205; RPL3; RPLPO; AAAS; RPL10A; RPL6; RPL7; |
| (GO: 0016032) | | | | | RPS14; RPL7A; RXRA; TPR; RPL14; RPS3; CCL2; RPL15; NUP214; |
| | | | | | IST1; RPS5; RPL13A; RPSA; NUP153; RPL23A; AP2B1; HCFC1; |
| | | | | | NUP93; VAPB; EIF3G; UBAP1; CHMP4A; NUP98; CD247; DOCK2; |
| | | | | | RAN; RPS23; EIF3B |
| regulation of | 7/29 | 6.63E−02 | 9.38E−01 | 2.19 | RMI2; RACGAP1; SPAG5; NEK2; CDC6; ECT2; PUM2 |
| chromosome | | | | | |
| segregation | | | | | |
| (GO: 0051983) | | | | | |
| response to molecule of | 18/98 | 6.71E−02 | 9.38E−01 | 1.55 | CXCL9; F2R; LY96; TNFAIP3; PTPN22; BCL10; TNFRSF1B; CXCL5; |
| bacterial origin | | | | | SIRT2; PRDX3; CYP27B1; IL6; SRR; IRAK1; TLR9; FAS; LTBR; |
| (GO: 0002237) | | | | | HNRNPA0 |
| regulation of intrinsic | 10/47 | 6.75E−02 | 9.38E−01 | 1.86 | NDUFA13; BCL2L11; BCL2A1; SRC; DAPK2; SIAH1; PLAUR; |
| apoptotic signaling | | | | | PMAIP1; BAX; BCL2L1 |
| pathway (GO: 2001242) | | | | | |
| positive regulation of | 10/47 | 6.75E−02 | 9.38E−01 | 1.86 | KAT2B; TAF1B; MYBBP1A; CHEK1; POLR1C; SMARCA5; |
| gene expression, | | | | | DDX21; CD3EAP; BAZ1B; SF3B1 |
| epigenetic | | | | | |
| (GO: 0045815) | | | | | |
| fatty acid biosynthetic | 10/47 | 6.75E−02 | 9.38E−01 | 1.86 | FADS3; ACLY; ABHD3; ELOVL5; ABHD1; SCD5; ACSM6; |
| process (GO: 0006633) | | | | | ACSM5; HACD2; CBR4 |
| regulation of | 8/35 | 6.80E−02 | 9.38E−01 | 2.04 | PRDX3; PMAIP1; BAX; MAPT; SLC25A33; SLC25A36; SOD1; |
| mitochondrial | | | | | BCL2L1 |
| membrane potential | | | | | |
| (GO: 0051881) | | | | | |
| regulation of | 9/41 | 6.82E−02 | 9.38E−01 | 1.94 | IDI1; FDPS; MVK; GPAM; PMVK; SC5D; SCAP; DHCR7; RAN |
| cholesterol metabolic | | | | | |
| process (GO: 0090181) | | | | | |
| histone H2A acetylation | 4/16 | 1.36E−01 | 9.93E−01 | 2.29 | TRRAP; RUVBL1; MSL3; BRD8 |
| (GO: 0043968) | | | | | |

TABLE 19
BCL2-KD-mediated LipocyteProfiler in subcutaneous AMSCs at day14. (ANOVA
adj. BMI, sex, age, batch, significance level 5% FDR). P-value, p-value of ANOVA, q-value,
q-value of ANOVA, FDR; eta_sq, eta square of ANOVA, effect size; F value of ANOVA; t-statistics of t-test.

| Lipocyte Profiler features | p-value | q-value | eta_sq | t-statistics |
|---|---|---|---|---|
| Cells_AreaShape_Compactness | 3.15E−03 | 3.42E−03 | 0.66 | 3.91 |
| Cells_AreaShape_Extent | 2.58E−03 | 2.94E−03 | 0.68 | −4.14 |
| Cells_AreaShape_MajorAxisLength | 1.07E−02 | 9.33E−03 | 0.56 | 3.16 |
| Cells_AreaShape_MaxFeretDiameter | 8.72E−03 | 7.97E−03 | 0.58 | 3.31 |
| Cells_AreaShape_Solidity | 2.40E−03 | 2.78E−03 | 0.67 | −4.02 |
| Cells_Children_LargeBODIPYObjects_Count | 2.49E−03 | 2.85E−03 | 0.46 | −2.59 |
| Cells_Correlation_Correlation_DNA_Lipid | 4.31E−07 | 1.23E−05 | 0.69 | 4.23 |
| Cells_Correlation_Correlation_Lipid_AGP | 4.69E−04 | 7.64E−04 | 0.30 | 1.84 |
| Cells_Correlation_Correlation_Mito_Lipid | 6.59E−04 | 9.78E−04 | 0.28 | 1.77 |
| Cells_Correlation_K_AGP_Lipid | 1.08E−06 | 1.40E−05 | 0.97 | −15.12 |
| Cells_Correlation_K_AGP_Mito | 1.20E−03 | 1.58E−03 | 0.51 | −2.86 |
| Cells_Correlation_K_DNA_Lipid | 2.50E−05 | 7.55E−05 | 0.82 | −6.05 |
| Cells_Correlation_K_DNA_Mito | 3.48E−03 | 3.72E−03 | 0.73 | −4.61 |
| Cells_Correlation_K_Lipid_AGP | 5.11E−06 | 2.41E−05 | 0.93 | 10.41 |
| Cells_Correlation_K_Lipid_DNA | 4.95E−05 | 1.30E−04 | 0.77 | 5.14 |
| Cells_Correlation_K_Lipid_Mito | 1.82E−04 | 3.67E−04 | 0.54 | 3.07 |
| Cells_Correlation_K_Mito_AGP | 9.05E−05 | 2.13E−04 | 0.41 | 2.37 |
| Cells_Correlation_K_Mito_DNA | 9.51E−03 | 8.50E−03 | 0.43 | 2.43 |
| Cells_Correlation_K_Mito_Lipid | 1.01E−04 | 2.32E−04 | 0.76 | −5.05 |
| Cells_Correlation_Overlap_DNA_Lipid | 5.96E−06 | 2.71E−05 | 0.81 | 5.87 |
| Cells_Correlation_Overlap_DNA_Mito | 1.07E−04 | 2.43E−04 | 0.60 | −3.44 |
| Cells_Correlation_Overlap_Lipid_AGP | 5.99E−03 | 5.87E−03 | 0.30 | 1.86 |
| Cells_Granularity_1_Lipid | 4.76E−04 | 7.69E−04 | 0.66 | 3.91 |
| Cells_Granularity_10_Lipid | 1.34E−03 | 1.73E−03 | 0.79 | −5.44 |
| Cells_Granularity_11_Lipid | 4.57E−03 | 4.66E−03 | 0.69 | −4.18 |
| Cells_Granularity_12_Lipid | 1.52E−03 | 1.91E−03 | 0.74 | −4.78 |
| Cells_Granularity_13_Lipid | 4.56E−03 | 4.65E−03 | 0.65 | −3.88 |
| Cells_Granularity_14_Lipid | 6.59E−03 | 6.34E−03 | 0.66 | −3.94 |
| Cells_Granularity_15_Lipid | 5.91E−03 | 5.80E−03 | 0.30 | −1.83 |
| Cells_Granularity_16_AGP | 1.07E−02 | 9.34E−03 | 0.38 | −2.21 |
| Cells_Granularity_2_Mito | 2.67E−03 | 3.03E−03 | 0.71 | 4.43 |
| Cells_Granularity_6_Lipid | 1.04E−02 | 9.14E−03 | 0.63 | −3.69 |
| Cells_Granularity_7_AGP | 3.43E−03 | 3.67E−03 | 0.56 | −3.17 |
| Cells_Granularity_7_Lipid | 1.96E−04 | 3.80E−04 | 0.86 | −7.06 |
| Cells_Granularity_8_Lipid | 6.58E−05 | 1.63E−04 | 0.86 | −7.02 |
| Cells_Granularity_9_Lipid | 1.18E−03 | 1.56E−03 | 0.74 | −4.75 |
| Cells_Intensity_IntegratedIntensity_Lipid | 1.56E−05 | 5.29E−05 | 0.83 | −6.30 |
| Cells_Intensity_IntegratedIntensityEdge_Lipid | 5.82E−04 | 8.84E−04 | 0.64 | −3.73 |
| Cells_Intensity_LowerQuartileIntensity_Lipid | 6.12E−05 | 1.55E−04 | 0.78 | −5.26 |
| Cells_Intensity_LowerQuartileIntensity_Mito | 6.61E−05 | 1.63E−04 | 0.89 | −8.00 |
| Cells_Intensity_MADIntensity_Lipid | 2.44E−06 | 1.59E−05 | 0.90 | −8.48 |
| Cells_Intensity_MADIntensity_Mito | 6.33E−04 | 9.45E−04 | 0.83 | −6.25 |
| Cells_Intensity_MassDisplacement_Mito | 7.62E−03 | 7.13E−03 | 0.49 | 2.76 |
| Cells_Intensity_MaxIntensity_Lipid | 2.18E−08 | 9.16E−06 | 0.90 | −8.56 |
| Cells_Intensity_MaxIntensity_Mito | 2.06E−03 | 2.45E−03 | 0.56 | −3.17 |
| Cells_Intensity_MaxIntensityEdge_Lipid | 4.00E−07 | 1.21E−05 | 0.83 | −6.13 |
| Cells_Intensity_MaxIntensityEdge_Mito | 2.05E−03 | 2.44E−03 | 0.76 | −5.05 |
| Cells_Intensity_MeanIntensity_Lipid | 2.21E−06 | 1.55E−05 | 0.87 | −7.18 |
| Cells_Intensity_MeanIntensity_Mito | 1.26E−04 | 2.77E−04 | 0.89 | −8.06 |
| Cells_Intensity_MeanIntensityEdge_DNA | 7.29E−03 | 6.86E−03 | 0.45 | −2.57 |
| Cells_Intensity_MeanIntensityEdge_Lipid | 5.68E−05 | 1.45E−04 | 0.73 | −4.66 |
| Cells_Intensity_MeanIntensityEdge_Mito | 6.25E−05 | 1.57E−04 | 0.86 | −7.05 |
| Cells_Intensity_MedianIntensity_Lipid | 1.73E−05 | 5.66E−05 | 0.84 | −6.40 |
| Cells_Intensity_MedianIntensity_Mito | 1.92E−04 | 3.76E−04 | 0.87 | −7.45 |
| Cells_Intensity_MinIntensity_Lipid | 1.96E−04 | 3.79E−04 | 0.70 | −4.28 |
| Cells_Intensity_MinIntensity_Mito | 8.31E−07 | 1.40E−05 | 0.97 | −15.41 |
| Cells_Intensity_MinIntensityEdge_Lipid | 9.67E−04 | 1.33E−03 | 0.59 | −3.39 |
| Cells_Intensity_MinIntensityEdge_Mito | 8.89E−07 | 1.40E−05 | 0.97 | −15.18 |
| Cells_Intensity_StdIntensity_Lipid | 2.65E−07 | 1.19E−05 | 0.89 | −8.02 |
| Cells_Intensity_StdIntensity_Mito | 2.83E−04 | 5.15E−04 | 0.75 | −4.90 |
| Cells_Intensity_StdIntensityEdge_Lipid | 6.58E−06 | 2.88E−05 | 0.80 | −5.64 |
| Cells_Intensity_StdIntensityEdge_Mito | 5.66E−04 | 8.68E−04 | 0.83 | −6.24 |
| Cells_Intensity_UpperQuartileIntensity_Lipid | 2.65E−06 | 1.69E−05 | 0.87 | −7.36 |
| Cells_Intensity_UpperQuartileIntensity_Mito | 2.52E−04 | 4.69E−04 | 0.87 | −7.24 |
| Cells_Mean_LargeBODIPYObjects_AreaShape_Area | 3.27E−04 | 5.77E−04 | 0.65 | −3.85 |
| Cells_Mean_LargeBODIPYObjects_AreaShape_Center_X | 9.51E−05 | 2.21E−04 | 0.61 | −3.52 |
| Cells_Mean_LargeBODIPYObjects_AreaShape_Center_Y | 3.67E−05 | 1.03E−04 | 0.67 | −4.02 |
| Cells_Mean_LargeBODIPYObjects_AreaShape_Center_Z | 4.78E−05 | 1.27E−04 | 0.62 | −3.59 |
| Cells_Mean_LargeBODIPYObjects_AreaShape_Compactness | 2.91E−05 | 8.60E−05 | 0.63 | −3.69 |
| Cells_Mean_LargeBODIPYObjects_AreaShape_Eccentricity | 2.74E−05 | 8.19E−05 | 0.64 | −3.74 |
| Cells_Mean_LargeBODIPYObjects_AreaShape_EulerNumber | 4.80E−05 | 1.27E−04 | 0.62 | −3.59 |
| Cells_Mean_LargeBODIPYObjects_AreaShape_Extent | 1.13E−04 | 2.53E−04 | 0.60 | −3.48 |
| Cells_Mean_LargeBODIPYObjects_AreaShape_FormFactor | 1.45E−04 | 3.09E−04 | 0.61 | −3.50 |
| Cells_Mean_LargeBODIPYObjects_AreaShape_MajorAxisLength | 6.01E−05 | 1.52E−04 | 0.65 | −3.84 |
| Cells_Mean_LargeBODIPYObjects_AreaShape_MaxFeretDiameter | 6.10E−05 | 1.54E−04 | 0.64 | −3.79 |
| Cells_Mean_LargeBODIPYObjects_AreaShape_MaximumRadius | 2.13E−04 | 4.08E−04 | 0.62 | −3.57 |
| Cells_Mean_LargeBODIPYObjects_AreaShape_MeanRadius | 1.89E−04 | 3.73E−04 | 0.62 | −3.62 |
| Cells_Mean_LargeBODIPYObjects_AreaShape_MedianRadius | 1.87E−04 | 3.72E−04 | 0.62 | −3.63 |
| Cells_Mean_LargeBODIPYObjects_AreaShape_MinFeretDiameter | 1.21E−04 | 2.69E−04 | 0.63 | −3.70 |
| Cells_Mean_LargeBODIPYObjects_AreaShape_MinorAxisLength | 1.42E−04 | 3.05E−04 | 0.63 | −3.68 |
| Cells_Mean_LargeBODIPYObjects_AreaShape_Perimeter | 6.52E−05 | 1.63E−04 | 0.64 | −3.75 |
| Cells_Mean_LargeBODIPYObjects_AreaShape_Solidity | 7.52E−05 | 1.81E−04 | 0.61 | −3.55 |
| Cells_Mean_LargeBODIPYObjects_Correlation_Correlation_DNA_AGP | 3.60E−04 | 6.18E−04 | 0.54 | −3.04 |
| Cells_Mean_LargeBODIPYObjects_Correlation_Correlation_DNA_Mito | 7.38E−05 | 1.78E−04 | 0.60 | −3.45 |
| Cells_Mean_LargeBODIPYObjects_Correlation_Correlation_Mito_AGP | 3.59E−04 | 6.17E−04 | 0.56 | −3.17 |
| Cells_Mean_LargeBODIPYObjects_Correlation_K_AGP_DNA | 2.46E−03 | 2.83E−03 | 0.72 | −4.48 |
| Cells_Mean_LargeBODIPYObjects_Correlation_K_AGP_Lipid | 9.37E−05 | 2.19E−04 | 0.87 | −7.44 |
| Cells_Mean_LargeBODIPYObjects_Correlation_K_AGP_Mito | 2.32E−06 | 1.56E−05 | 0.93 | −10.02 |
| Cells_Mean_LargeBODIPYObjects_Correlation_K_DNA_Lipid | 5.10E−05 | 1.33E−04 | 0.77 | −5.10 |
| Cells_Mean_LargeBODIPYObjects_Correlation_K_DNA_Mito | 9.25E−04 | 1.29E−03 | 0.64 | −3.74 |
| Cells_Mean_LargeBODIPYObjects_Correlation_K_Mito_Lipid | 1.56E−03 | 1.96E−03 | 0.60 | −3.49 |
| Cells_Mean_LargeBODIPYObjects_Correlation_Overlap_DNA_AGP | 4.76E−05 | 1.27E−04 | 0.62 | −3.63 |
| Cells_Mean_LargeBODIPYObjects_Correlation_Overlap_DNA_Lipid | 3.78E−05 | 1.05E−04 | 0.65 | −3.81 |
| Cells_Mean_LargeBODIPYObjects_Correlation_Overlap_DNA_Mito | 4.21E−05 | 1.15E−04 | 0.63 | −3.68 |
| Cells_Mean_LargeBODIPYObjects_Correlation_Overlap_Lipid_AGP | 3.78E−05 | 1.05E−04 | 0.65 | −3.84 |
| Cells_Mean_LargeBODIPYObjects_Correlation_Overlap_Mito_AGP | 4.56E−05 | 1.24E−04 | 0.63 | −3.68 |
| Cells_Mean_LargeBODIPYObjects_Correlation_Overlap_Mito_Lipid | 3.46E−05 | 9.86E−05 | 0.66 | −3.94 |
| Cells_Mean_LargeBODIPYObjects_Granularity_10_Lipid | 1.50E−04 | 3.16E−04 | 0.69 | −4.26 |
| Cells_Mean_LargeBODIPYObjects_Granularity_11_Lipid | 3.91E−04 | 6.61E−04 | 0.62 | −3.60 |
| Cells_Mean_LargeBODIPYObjects_Granularity_12_Lipid | 2.81E−04 | 5.12E−04 | 0.60 | −3.45 |
| Cells_Mean_LargeBODIPYObjects_Granularity_13_Lipid | 7.26E−04 | 1.06E−03 | 0.62 | −3.58 |
| Cells_Mean_LargeBODIPYObjects_Granularity_14_Lipid | 1.10E−03 | 1.47E−03 | 0.66 | −3.95 |
| Cells_Mean_LargeBODIPYObjects_Granularity_15_Lipid | 3.70E−03 | 3.92E−03 | 0.48 | −2.70 |
| Cells_Mean_LargeBODIPYObjects_Granularity_16_Lipid | 4.75E−03 | 4.82E−03 | 0.50 | −2.83 |
| Cells_Mean_LargeBODIPYObjects_Granularity_5_Lipid | 5.20E−03 | 5.19E−03 | 0.49 | −2.78 |
| Cells_Mean_LargeBODIPYObjects_Granularity_6_Lipid | 3.41E−04 | 5.97E−04 | 0.69 | −4.18 |
| Cells_Mean_LargeBODIPYObjects_Granularity_7_Lipid | 3.31E−05 | 9.53E−05 | 0.75 | −4.92 |
| Cells_Mean_LargeBODIPYObjects_Granularity_8_Lipid | 1.28E−05 | 4.63E−05 | 0.71 | −4.47 |
| Cells_Mean_LargeBODIPYObjects_Granularity_9_Lipid | 1.81E−04 | 3.65E−04 | 0.63 | −3.70 |
| Cells_Mean_LargeBODIPYObjects_Intensity_IntegratedIntensity_Lipid | 1.02E−04 | 2.34E−04 | 0.76 | −5.07 |
| Cells_Mean_LargeBODIPYObjects_Intensity_IntegratedIntensityEdge_Lipid | 7.93E−05 | 1.89E−04 | 0.76 | −4.97 |
| Cells_Mean_LargeBODIPYObjects_Intensity_LowerQuartileIntensity_Lipid | 1.35E−04 | 2.90E−04 | 0.73 | −4.62 |
| Cells_Mean_LargeBODIPYObjects_Intensity_MADIntensity_Lipid | 8.26E−05 | 1.96E−04 | 0.72 | −4.52 |
| Cells_Mean_LargeBODIPYObjects_Intensity_MassDisplacement_Lipid | 1.24E−04 | 2.74E−04 | 0.67 | −4.02 |
| Cells_Mean_LargeBODIPYObjects_Intensity_MaxIntensity_Lipid | 2.90E−05 | 8.60E−05 | 0.77 | −5.10 |
| Cells_Mean_LargeBODIPYObjects_Intensity_MaxIntensityEdge_Lipid | 3.20E−05 | 9.36E−05 | 0.77 | −5.19 |
| Cells_Mean_LargeBODIPYObjects_Intensity_MeanIntensity_Lipid | 6.60E−05 | 1.63E−04 | 0.75 | −4.88 |
| Cells_Mean_LargeBODIPYObjects_Intensity_MeanIntensityEdge_Lipid | 6.17E−05 | 1.55E−04 | 0.76 | −4.98 |
| Cells_Mean_LargeBODIPYObjects_Intensity_MedianIntensity_Lipid | 7.48E−05 | 1.80E−04 | 0.75 | −4.89 |
| Cells_Mean_LargeBODIPYObjects_Intensity_MinIntensity_Lipid | 3.43E−05 | 9.80E−05 | 0.76 | −5.09 |
| Cells_Mean_LargeBODIPYObjects_Intensity_MinIntensityEdge_Lipid | 3.57E−05 | 1.01E−04 | 0.76 | −5.07 |
| Cells_Mean_LargeBODIPYObjects_Intensity_StdIntensity_Lipid | 2.31E−05 | 7.13E−05 | 0.79 | −5.44 |
| Cells_Mean_LargeBODIPYObjects_Intensity_StdIntensityEdge_Lipid | 5.02E−05 | 1.31E−04 | 0.79 | −5.41 |
| Cells_Mean_LargeBODIPYObjects_Intensity_UpperQuartileIntensity_Lipid | 4.58E−05 | 1.24E−04 | 0.76 | −5.01 |
| Cells_Mean_LargeBODIPYObjects_Location_Center_X | 9.52E−05 | 2.21E−04 | 0.61 | −3.52 |
| Cells_Mean_LargeBODIPYObjects_Location_Center_Y | 3.68E−05 | 1.03E−04 | 0.67 | −4.02 |
| Cells_Mean_LargeBODIPYObjects_Location_CenterMassIntensity_X_Lipid | 9.53E−05 | 2.21E−04 | 0.61 | −3.52 |
| Cells_Mean_LargeBODIPYObjects_Location_CenterMassIntensity_Y_Lipid | 3.68E−05 | 1.03E−04 | 0.67 | −4.02 |
| Cells_Mean_LargeBODIPYObjects_Location_MaxIntensity_X_Lipid | 9.55E−05 | 2.21E−04 | 0.61 | −3.52 |
| Cells_Mean_LargeBODIPYObjects_Location_MaxIntensity_Y_Lipid | 3.68E−05 | 1.03E−04 | 0.67 | −4.02 |
| Cells_Neighbors_AngleBetweenNeighbors_Adjacent | 2.44E−03 | 2.81E−03 | 0.51 | −2.91 |
| Cells_Neighbors_FirstClosestDistance_Adjacent | 6.76E−03 | 6.47E−03 | 0.48 | 2.74 |
| Cells_Neighbors_FirstClosestObjectNumber_Adjacent | 1.62E−03 | 2.02E−03 | 0.55 | −3.12 |
| Cells_Neighbors_SecondClosestDistance_Adjacent | 4.35E−03 | 4.48E−03 | 0.50 | 2.85 |
| Cells_Neighbors_SecondClosestObjectNumber_Adjacent | 1.62E−03 | 2.02E−03 | 0.55 | −3.12 |
| Cells_Number_Object_Number | 1.64E−03 | 2.02E−03 | 0.55 | −3.13 |
| Cells_Parent_Nuclei | 1.64E−03 | 2.02E−03 | 0.55 | −3.13 |
| Cytoplasm_AreaShape_FormFactor | 5.58E−03 | 5.51E−03 | 0.40 | −2.33 |
| Cytoplasm_AreaShape_MajorAxisLength | 8.95E−03 | 8.14E−03 | 0.57 | 3.24 |
| Cytoplasm_AreaShape_MaxFeretDiameter | 8.72E−03 | 7.97E−03 | 0.58 | 3.31 |
| Cytoplasm_Correlation_Correlation_DNA_Lipid | 1.90E−06 | 1.54E−05 | 0.53 | 3.00 |
| Cytoplasm_Correlation_K_AGP_Lipid | 2.36E−06 | 1.57E−05 | 0.96 | −14.36 |
| Cytoplasm_Correlation_K_AGP_Mito | 8.63E−04 | 1.21E−03 | 0.63 | −3.65 |
| Cytoplasm_Correlation_K_DNA_Lipid | 9.57E−06 | 3.82E−05 | 0.85 | −6.64 |
| Cytoplasm_Correlation_K_DNA_Mito | 6.81E−03 | 6.51E−03 | 0.67 | −4.05 |
| Cytoplasm_Correlation_K_Lipid_AGP | 2.78E−06 | 1.72E−05 | 0.90 | 8.26 |
| Cytoplasm_Correlation_K_Lipid_DNA | 2.05E−05 | 6.59E−05 | 0.78 | 5.33 |
| Cytoplasm_Correlation_K_Lipid_Mito | 2.92E−05 | 8.61E−05 | 0.61 | 3.52 |
| Cytoplasm_Correlation_K_Mito_AGP | 1.14E−04 | 2.55E−04 | 0.54 | 3.07 |
| Cytoplasm_Correlation_K_Mito_DNA | 1.58E−03 | 1.98E−03 | 0.59 | 3.38 |
| Cytoplasm_Correlation_K_Mito_Lipid | 1.90E−04 | 3.74E−04 | 0.78 | −5.28 |
| Cytoplasm_Correlation_Overlap_DNA_Lipid | 1.92E−03 | 2.31E−03 | 0.51 | 2.88 |
| Cytoplasm_Correlation_Overlap_DNA_Mito | 2.65E−04 | 4.89E−04 | 0.78 | −5.31 |
| Cytoplasm_Granularity_1_Lipid | 5.49E−04 | 8.50E−04 | 0.63 | 3.71 |
| Cytoplasm_Granularity_10_Lipid | 1.26E−04 | 2.77E−04 | 0.84 | −6.45 |
| Cytoplasm_Granularity_11_Lipid | 3.16E−04 | 5.64E−04 | 0.79 | −5.44 |
| Cytoplasm_Granularity_12_Lipid | 3.11E−04 | 5.59E−04 | 0.82 | −5.95 |
| Cytoplasm_Granularity_13_Lipid | 8.57E−04 | 1.21E−03 | 0.80 | −5.67 |
| Cytoplasm_Granularity_14_Lipid | 2.43E−03 | 2.80E−03 | 0.75 | −4.86 |
| Cytoplasm_Granularity_16_AGP | 1.04E−02 | 9.14E−03 | 0.38 | −2.21 |
| Cytoplasm_Granularity_2_Mito | 4.35E−05 | 1.19E−04 | 0.86 | 6.95 |
| Cytoplasm_Granularity_6_Lipid | 2.45E−03 | 2.82E−03 | 0.71 | −4.43 |
| Cytoplasm_Granularity_7_AGP | 1.09E−02 | 9.52E−03 | 0.63 | −3.67 |
| Cytoplasm_Granularity_7_Lipid | 4.18E−05 | 1.15E−04 | 0.85 | −6.70 |
| Cytoplasm_Granularity_8_Lipid | 8.75E−06 | 3.63E−05 | 0.84 | −6.57 |
| Cytoplasm_Granularity_9_Lipid | 1.07E−04 | 2.43E−04 | 0.76 | −4.97 |
| Cytoplasm_Intensity_IntegratedIntensity_Lipid | 7.14E−04 | 1.05E−03 | 0.66 | −3.94 |
| Cytoplasm_Intensity_IntegratedIntensityEdge_Lipid | 2.89E−05 | 8.58E−05 | 0.79 | −5.42 |
| Cytoplasm_Intensity_LowerQuartileIntensity_DNA | 1.04E−02 | 9.14E−03 | 0.43 | −2.45 |
| Cytoplasm_Intensity_LowerQuartileIntensity_Lipid | 1.18E−03 | 1.55E−03 | 0.62 | −3.58 |
| Cytoplasm_Intensity_LowerQuartileIntensity_Mito | 3.26E−05 | 9.45E−05 | 0.86 | −7.13 |
| Cytoplasm_Intensity_MADIntensity_DNA | 4.89E−03 | 4.94E−03 | 0.58 | −3.31 |
| Cytoplasm_Intensity_MADIntensity_Lipid | 2.78E−06 | 1.72E−05 | 0.84 | −6.48 |
| Cytoplasm_Intensity_MADIntensity_Mito | 5.87E−04 | 8.88E−04 | 0.76 | −5.00 |
| Cytoplasm_Intensity_MassDisplacement_DNA | 8.82E−03 | 8.05E−03 | 0.54 | 3.07 |
| Cytoplasm_Intensity_MassDisplacement_Mito | 6.70E−03 | 6.43E−03 | 0.54 | 3.05 |
| Cytoplasm_Intensity_MaxIntensity_Lipid | 9.80E−08 | 9.16E−06 | 0.88 | −7.59 |
| Cytoplasm_Intensity_MaxIntensityEdge_Lipid | 1.20E−07 | 9.16E−06 | 0.87 | −7.35 |
| Cytoplasm_Intensity_MaxIntensityEdge_Mito | 3.95E−03 | 4.15E−03 | 0.66 | −3.97 |
| Cytoplasm_Intensity_MeanIntensity_Lipid | 1.07E−05 | 4.09E−05 | 0.81 | −5.88 |
| Cytoplasm_Intensity_MeanIntensity_Mito | 1.48E−04 | 3.14E−04 | 0.84 | −6.51 |
| Cytoplasm_Intensity_MeanIntensityEdge_Lipid | 1.40E−05 | 4.84E−05 | 0.81 | −5.79 |
| Cytoplasm_Intensity_MeanIntensityEdge_Mito | 7.47E−05 | 1.80E−04 | 0.88 | −7.64 |
| Cytoplasm_Intensity_MedianIntensity_DNA | 7.07E−03 | 6.71E−03 | 0.50 | −2.84 |
| Cytoplasm_Intensity_MedianIntensity_Lipid | 2.37E−04 | 4.45E−04 | 0.72 | −4.54 |
| Cytoplasm_Intensity_MedianIntensity_Mito | 9.14E−05 | 2.15E−04 | 0.83 | −6.28 |
| Cytoplasm_Intensity_MinIntensity_Lipid | 4.35E−04 | 7.20E−04 | 0.65 | −3.81 |
| Cytoplasm_Intensity_MinIntensity_Mito | 8.31E−07 | 1.40E−05 | 0.97 | −15.41 |
| Cytoplasm_Intensity_MinIntensityEdge_Lipid | 4.67E−04 | 7.62E−04 | 0.64 | −3.77 |
| Cytoplasm_Intensity_MinIntensityEdge_Mito | 8.87E−07 | 1.40E−05 | 0.97 | −15.19 |
| Cytoplasm_Intensity_StdIntensity_Lipid | 2.96E−07 | 1.19E−05 | 0.87 | −7.37 |
| Cytoplasm_Intensity_StdIntensity_Mito | 2.78E−03 | 3.11E−03 | 0.74 | −4.77 |
| Cytoplasm_Intensity_StdIntensityEdge_Lipid | 8.03E−07 | 1.40E−05 | 0.87 | −7.20 |
| Cytoplasm_Intensity_StdIntensityEdge_Mito | 9.93E−04 | 1.36E−03 | 0.81 | −5.76 |
| Cytoplasm_Intensity_UpperQuartileIntensity_DNA | 9.01E−03 | 8.18E−03 | 0.51 | −2.88 |
| Cytoplasm_Intensity_UpperQuartileIntensity_Lipid | 9.30E−06 | 3.79E−05 | 0.83 | −6.14 |
| Cytoplasm_Intensity_UpperQuartileIntensity_Mito | 2.21E−04 | 4.21E−04 | 0.81 | −5.93 |
| Cytoplasm_Number_Object_Number | 1.64E−03 | 2.02E−03 | 0.55 | −3.13 |
| Cytoplasm_Parent_Cells | 1.64E−03 | 2.02E−03 | 0.55 | −3.13 |
| Cytoplasm_Parent_Nuclei | 1.64E−03 | 2.02E−03 | 0.55 | −3.13 |
| Nuclei_AreaShape_Extent | 1.11E−02 | 9.57E−03 | 0.31 | 1.88 |
| Nuclei_AreaShape_FormFactor | 6.69E−03 | 6.42E−03 | 0.27 | 1.72 |
| Nuclei_AreaShape_MaximumRadius | 4.35E−03 | 4.48E−03 | 0.46 | 2.63 |
| Nuclei_AreaShape_MeanRadius | 1.69E−03 | 2.08E−03 | 0.45 | 2.58 |
| Nuclei_AreaShape_MedianRadius | 1.46E−03 | 1.85E−03 | 0.44 | 2.52 |
| Nuclei_AreaShape_Solidity | 1.76E−03 | 2.15E−03 | 0.30 | 1.86 |
| Nuclei_Correlation_Correlation_DNA_Lipid | 5.87E−07 | 1.40E−05 | 0.80 | 5.65 |
| Nuclei_Correlation_Correlation_Lipid_AGP | 9.38E−04 | 1.30E−03 | 0.43 | 2.45 |
| Nuclei_Correlation_K_AGP_Lipid | 2.74E−06 | 1.72E−05 | 0.96 | −14.36 |
| Nuclei_Correlation_K_AGP_Mito | 6.71E−04 | 9.93E−04 | 0.54 | −3.08 |
| Nuclei_Correlation_K_DNA_Lipid | 2.99E−06 | 1.74E−05 | 0.89 | −7.83 |
| Nuclei_Correlation_K_DNA_Mito | 3.09E−03 | 3.37E−03 | 0.74 | −4.72 |
| Nuclei_Correlation_K_Lipid_AGP | 5.79E−06 | 2.66E−05 | 0.95 | 11.79 |
| Nuclei_Correlation_K_Lipid_DNA | 4.77E−05 | 1.27E−04 | 0.78 | 5.32 |
| Nuclei_Correlation_K_Lipid_Mito | 1.51E−04 | 3.16E−04 | 0.56 | 3.18 |
| Nuclei_Correlation_K_Mito_AGP | 5.26E−05 | 1.36E−04 | 0.42 | 2.42 |
| Nuclei_Correlation_K_Mito_DNA | 8.42E−03 | 7.72E−03 | 0.43 | 2.48 |
| Nuclei_Correlation_K_Mito_Lipid | 1.07E−04 | 2.43E−04 | 0.80 | −5.58 |
| Nuclei_Correlation_Overlap_DNA_Lipid | 2.70E−05 | 8.13E−05 | 0.85 | 6.73 |
| Nuclei_Correlation_Overlap_DNA_Mito | 1.25E−04 | 2.77E−04 | 0.59 | −3.35 |
| Nuclei_Correlation_Overlap_Lipid_AGP | 5.16E−04 | 8.16E−04 | 0.49 | 2.79 |
| Nuclei_Granularity_1_Lipid | 3.37E−03 | 3.61E−03 | 0.47 | 2.64 |
| Nuclei_Granularity_11_DNA | 3.99E−03 | 4.19E−03 | 0.17 | 1.30 |
| Nuclei_Granularity_2_Mito | 8.14E−03 | 7.53E−03 | 0.58 | 3.33 |
| Nuclei_Granularity_8_Lipid | 8.12E−03 | 7.52E−03 | 0.65 | −3.87 |
| Nuclei_Intensity_IntegratedIntensity_Lipid | 1.99E−04 | 3.85E−04 | 0.87 | −7.36 |
| Nuclei_Intensity_IntegratedIntensityEdge_Lipid | 2.45E−06 | 1.59E−05 | 0.85 | −6.81 |
| Nuclei_Intensity_IntegratedIntensityEdge_Mito | 1.10E−02 | 9.56E−03 | 0.63 | −3.66 |
| Nuclei_Intensity_LowerQuartileIntensity_Lipid | 8.48E−06 | 3.54E−05 | 0.88 | −7.62 |
| Nuclei_Intensity_LowerQuartileIntensity_Mito | 2.93E−04 | 5.32E−04 | 0.84 | −6.39 |
| Nuclei_Intensity_MADIntensity_Lipid | 6.46E−08 | 9.16E−06 | 0.95 | −11.69 |
| Nuclei_Intensity_MADIntensity_Mito | 9.61E−04 | 1.32E−03 | 0.46 | −2.63 |
| Nuclei_Intensity_MassDisplacement_Mito | 3.19E−03 | 3.47E−03 | 0.62 | 3.58 |
| Nuclei_Intensity_MaxIntensity_Lipid | 1.87E−08 | 9.16E−06 | 0.93 | −10.62 |
| Nuclei_Intensity_MaxIntensity_Mito | 1.16E−03 | 1.54E−03 | 0.56 | −3.21 |
| Nuclei_Intensity_MaxIntensityEdge_Lipid | 1.09E−07 | 9.16E−06 | 0.91 | −8.79 |
| Nuclei_Intensity_MaxIntensityEdge_Mito | 4.19E−03 | 4.35E−03 | 0.66 | −3.93 |
| Nuclei_Intensity_MeanIntensity_Lipid | 7.19E−07 | 1.40E−05 | 0.91 | −9.05 |
| Nuclei_Intensity_MeanIntensity_Mito | 3.18E−04 | 5.66E−04 | 0.77 | −5.23 |
| Nuclei_Intensity_MeanIntensityEdge_Lipid | 1.24E−05 | 4.51E−05 | 0.84 | −6.45 |
| Nuclei_Intensity_MeanIntensityEdge_Mito | 8.52E−04 | 1.21E−03 | 0.82 | −5.93 |
| Nuclei_Intensity_MedianIntensity_Lipid | 1.46E−06 | 1.54E−05 | 0.91 | −8.95 |
| Nuclei_Intensity_MedianIntensity_Mito | 3.48E−04 | 6.04E−04 | 0.78 | −5.27 |
| Nuclei_Intensity_MinIntensity_Lipid | 1.37E−03 | 1.76E−03 | 0.64 | −3.75 |
| Nuclei_Intensity_MinIntensity_Mito | 1.27E−04 | 2.79E−04 | 0.86 | −7.09 |
| Nuclei_Intensity_MinIntensityEdge_Lipid | 4.16E−03 | 4.33E−03 | 0.56 | −3.16 |
| Nuclei_Intensity_MinIntensityEdge_Mito | 1.34E−04 | 2.90E−04 | 0.86 | −7.02 |
| Nuclei_Intensity_StdIntensity_Lipid | 8.16E−08 | 9.16E−06 | 0.94 | −10.69 |
| Nuclei_Intensity_StdIntensity_Mito | 1.05E−03 | 1.42E−03 | 0.50 | −2.83 |
| Nuclei_Intensity_StdIntensityEdge_Lipid | 3.33E−08 | 9.16E−06 | 0.94 | −10.77 |
| Nuclei_Intensity_StdIntensityEdge_Mito | 1.13E−02 | 9.78E−03 | 0.54 | −3.03 |
| Nuclei_Intensity_UpperQuartileIntensity_Lipid | 5.19E−07 | 1.40E−05 | 0.92 | −9.44 |
| Nuclei_Intensity_UpperQuartileIntensity_Mito | 3.67E−04 | 6.26E−04 | 0.73 | −4.62 |
| Nuclei_Number_Object_Number | 1.64E−03 | 2.02E−03 | 0.55 | −3.13 |
| Cells_AreaShape_Zernike_4_4 | 4.82E−04 | 7.74E−04 | 0.81 | −5.87 |
| Cells_Neighbors_AngleBetweenNeighbors_10 | 2.44E−03 | 2.81E−03 | 0.51 | −2.91 |
| Cells_Neighbors_FirstClosestDistance_10 | 6.76E−03 | 6.47E−03 | 0.48 | 2.74 |
| Cells_Neighbors_FirstClosestObjectNumber_10 | 1.62E−03 | 2.02E−03 | 0.55 | −3.12 |
| Cells_Neighbors_PercentTouching_10 | 4.56E−08 | 9.16E−06 | 0.28 | −1.75 |
| Cells_Neighbors_SecondClosestDistance_10 | 4.35E−03 | 4.48E−03 | 0.50 | 2.85 |
| Cells_Neighbors_SecondClosestObjectNumber_10 | 1.62E−03 | 2.02E−03 | 0.55 | −3.12 |
| Cells_RadialDistribution_MeanFrac_DNA_1of4 | 9.45E−03 | 8.45E−03 | 0.47 | 2.64 |
| Cells_RadialDistribution_MeanFrac_Lipid_1of4 | 3.28E−05 | 9.48E−05 | 0.51 | 2.90 |
| Cells_RadialDistribution_MeanFrac_Mito_3of4 | 1.35E−05 | 4.79E−05 | 0.41 | 2.37 |
| Cells_RadialDistribution_RadialCV_Lipid_4of4 | 2.86E−03 | 3.20E−03 | 0.68 | −4.15 |
| Cells_RadialDistribution_RadialCV_Mito_2of4 | 1.04E−03 | 1.41E−03 | 0.79 | 5.42 |
| Cells_Texture_AngularSecondMoment_DNA_20_03 | 8.98E−03 | 8.16E−03 | 0.65 | 3.83 |
| Cells_Texture_AngularSecondMoment_Lipid_5_00 | 7.45E−04 | 1.09E−03 | 0.66 | 3.98 |
| Cells_Texture_AngularSecondMoment_Mito_5_00 | 5.39E−03 | 5.35E−03 | 0.50 | 2.82 |
| Cells_Texture_Contrast_Lipid_20_00 | 1.65E−06 | 1.54E−05 | 0.92 | −9.35 |
| Cells_Texture_Contrast_Mito_20_03 | 6.06E−04 | 9.11E−04 | 0.74 | −4.80 |
| Cells_Texture_DifferenceEntropy_Lipid_20_01 | 6.94E−06 | 3.02E−05 | 0.85 | −6.69 |
| Cells_Texture_DifferenceEntropy_Mito_20_03 | 5.66E−04 | 8.68E−04 | 0.83 | −6.35 |
| Cells_Texture_DifferenceVariance_Lipid_5_01 | 1.26E−04 | 2.77E−04 | 0.79 | 5.41 |
| Cells_Texture_DifferenceVariance_Mito_20_03 | 2.31E−03 | 2.69E−03 | 0.73 | 4.69 |
| Cells_Texture_Entropy_Lipid_20_03 | 2.48E−05 | 7.53E−05 | 0.81 | −5.82 |
| Cells_Texture_Entropy_Mito_20_03 | 3.22E−04 | 5.70E−04 | 0.83 | −6.26 |
| Cells_Texture_InfoMeas1_DNA_20_03 | 1.22E−03 | 1.60E−03 | 0.76 | −5.05 |
| Cells_Texture_InfoMeas1_Lipid_20_01 | 6.03E−07 | 1.40E−05 | 0.85 | 6.70 |
| Cells_Texture_InfoMeas2_Mito_20_01 | 1.76E−04 | 3.58E−04 | 0.76 | −4.98 |
| Cells_Texture_InverseDifferenceMoment_Lipid_10_03 | 2.20E−04 | 4.20E−04 | 0.77 | 5.13 |
| Cells_Texture_InverseDifferenceMoment_Mito_5_02 | 2.71E−03 | 3.05E−03 | 0.67 | 4.01 |
| Cells_Texture_SumAverage_Lipid_20_00 | 1.74E−06 | 1.54E−05 | 0.88 | −7.74 |
| Cells_Texture_SumAverage_Mito_20_03 | 7.64E−05 | 1.83E−04 | 0.90 | −8.57 |
| Cells_Texture_SumEntropy_Lipid_20_01 | 4.89E−06 | 2.35E−05 | 0.83 | −6.32 |
| Cells_Texture_SumEntropy_Mito_20_03 | 2.77E−04 | 5.09E−04 | 0.86 | −6.98 |
| Cells_Texture_SumVariance_Lipid_10_01 | 1.47E−06 | 1.54E−05 | 0.91 | −8.73 |
| Cells_Texture_SumVariance_Mito_5_02 | 3.66E−04 | 6.25E−04 | 0.73 | −4.66 |
| Cells_Texture_Variance_Lipid_20_00 | 1.54E−06 | 1.54E−05 | 0.91 | −8.79 |
| Cells_Texture_Variance_Mito_20_03 | 3.98E−04 | 6.69E−04 | 0.73 | −4.65 |
| Cytoplasm_AreaShape_Zernike_7_7 | 1.84E−04 | 3.68E−04 | 0.77 | −5.24 |
| Cytoplasm_RadialDistribution_FracAtD_AGP_1of4 | 4.83E−03 | 4.88E−03 | 0.34 | −2.03 |
| Cytoplasm_RadialDistribution_FracAtD_DNA_1of4 | 9.11E−04 | 1.28E−03 | 0.47 | −2.67 |
| Cytoplasm_RadialDistribution_FracAtD_Lipid_2of4 | 1.91E−03 | 2.30E−03 | 0.74 | −4.83 |
| Cytoplasm_RadialDistribution_FracAtD_Mito_2of4 | 4.41E−04 | 7.25E−04 | 0.40 | −2.32 |
| Cytoplasm_RadialDistribution_MeanFrac_Mito_2of4 | 2.87E−03 | 3.20E−03 | 0.32 | −1.94 |
| Cytoplasm_RadialDistribution_RadialCV_AGP_3of4 | 1.92E−03 | 2.31E−03 | 0.75 | −4.83 |
| Cytoplasm_RadialDistribution_RadialCV_Lipid_4of4 | 5.09E−04 | 8.06E−04 | 0.59 | −3.42 |
| Cytoplasm_RadialDistribution_RadialCV_Mito_3of4 | 2.95E−04 | 5.34E−04 | 0.77 | 5.19 |
| Cytoplasm_Texture_AngularSecondMoment_DNA_10_01 | 2.08E−03 | 2.47E−03 | 0.69 | 4.19 |
| Cytoplasm_Texture_AngularSecondMoment_Lipid_5_02 | 6.32E−05 | 1.58E−04 | 0.68 | 4.12 |
| Cytoplasm_Texture_AngularSecondMoment_Mito_5_02 | 2.17E−03 | 2.57E−03 | 0.53 | 2.99 |
| Cytoplasm_Texture_Contrast_Lipid_20_03 | 3.65E−06 | 1.89E−05 | 0.91 | −8.78 |
| Cytoplasm_Texture_Contrast_Mito_20_03 | 6.12E−03 | 5.97E−03 | 0.68 | −4.14 |
| Cytoplasm_Texture_Correlation_DNA_20_01 | 8.26E−03 | 7.61E−03 | 0.65 | 3.89 |
| Cytoplasm_Texture_Correlation_Lipid_5_02 | 2.90E−03 | 3.21E−03 | 0.46 | −2.61 |
| Cytoplasm_Texture_DifferenceEntropy_DNA_20_00 | 8.88E−03 | 8.09E−03 | 0.63 | −3.68 |
| Cytoplasm_Texture_DifferenceEntropy_Lipid_10_01 | 2.95E−06 | 1.74E−05 | 0.77 | −5.20 |
| Cytoplasm_Texture_DifferenceEntropy_Mito_20_01 | 1.06E−03 | 1.43E−03 | 0.70 | −4.28 |
| Cytoplasm_Texture_DifferenceVariance_Lipid_5_01 | 1.22E−05 | 4.48E−05 | 0.75 | 4.96 |
| Cytoplasm_Texture_DifferenceVariance_Mito_5_02 | 1.57E−03 | 1.96E−03 | 0.75 | 4.84 |
| Cytoplasm_Texture_Entropy_DNA_20_02 | 4.06E−03 | 4.24E−03 | 0.65 | −3.85 |
| Cytoplasm_Texture_Entropy_Lipid_10_01 | 2.85E−06 | 1.72E−05 | 0.74 | −4.79 |
| Cytoplasm_Texture_Entropy_Mito_5_02 | 7.37E−04 | 1.08E−03 | 0.68 | −4.16 |
| Cytoplasm_Texture_InfoMeas1_DNA_10_01 | 5.04E−03 | 5.05E−03 | 0.68 | −4.08 |
| Cytoplasm_Texture_InfoMeas1_Lipid_20_01 | 1.73E−05 | 5.66E−05 | 0.78 | 5.28 |
| Cytoplasm_Texture_InfoMeas2_Mito_5_00 | 8.37E−07 | 1.40E−05 | 0.97 | −15.59 |
| Cytoplasm_Texture_InverseDifferenceMoment_DNA_10_01 | 7.53E−03 | 7.05E−03 | 0.62 | 3.64 |
| Cytoplasm_Texture_InverseDifferenceMoment_Lipid_10_01 | 2.10E−05 | 6.68E−05 | 0.75 | 4.87 |
| Cytoplasm_Texture_InverseDifferenceMoment_Mito_5_02 | 1.91E−03 | 2.30E−03 | 0.64 | 3.76 |
| Cytoplasm_Texture_SumAverage_DNA_20_02 | 9.18E−03 | 8.29E−03 | 0.58 | −3.31 |
| Cytoplasm_Texture_SumAverage_Lipid_20_03 | 9.50E−06 | 3.81E−05 | 0.83 | −6.15 |
| Cytoplasm_Texture_SumAverage_Mito_20_03 | 1.28E−04 | 2.81E−04 | 0.85 | −6.63 |
| Cytoplasm_Texture_SumEntropy_DNA_5_02 | 3.31E−03 | 3.56E−03 | 0.66 | −3.90 |
| Cytoplasm_Texture_SumEntropy_Lipid_10_01 | 1.45E−06 | 1.54E−05 | 0.76 | −5.01 |
| Cytoplasm_Texture_SumEntropy_Mito_5_02 | 4.82E−04 | 7.74E−04 | 0.71 | −4.44 |
| Cytoplasm_Texture_SumVariance_Lipid_5_01 | 2.86E−06 | 1.72E−05 | 0.88 | −7.81 |
| Cytoplasm_Texture_SumVariance_Mito_20_03 | 6.11E−03 | 5.96E−03 | 0.68 | −4.14 |
| Cytoplasm_Texture_Variance_Lipid_20_03 | 4.02E−06 | 2.02E−05 | 0.89 | −7.84 |
| Cytoplasm_Texture_Variance_Mito_20_03 | 5.87E−03 | 5.77E−03 | 0.69 | −4.17 |
| Nuclei_AreaShape_Zernike_7_1 | 1.91E−04 | 3.75E−04 | 0.64 | −3.76 |
| Nuclei_Neighbors_AngleBetweenNeighbors_2 | 6.59E−03 | 6.34E−03 | 0.50 | −2.80 |
| Nuclei_Neighbors_FirstClosestDistance_2 | 5.62E−03 | 5.54E−03 | 0.52 | 2.96 |
| Nuclei_Neighbors_FirstClosestObjectNumber_2 | 1.66E−03 | 2.05E−03 | 0.55 | −3.12 |
| Nuclei_Neighbors_SecondClosestDistance_2 | 3.24E−03 | 3.51E−03 | 0.53 | 2.99 |
| Nuclei_Neighbors_SecondClosestObjectNumber_2 | 1.65E−03 | 2.03E−03 | 0.55 | −3.12 |
| Nuclei_RadialDistribution_FracAtD_Lipid_1of4 | 1.22E−06 | 1.46E−05 | 0.66 | 3.92 |
| Nuclei_RadialDistribution_MeanFrac_Lipid_1of4 | 2.00E−06 | 1.55E−05 | 0.71 | 4.37 |
| Nuclei_RadialDistribution_RadialCV_DNA_1of4 | 1.36E−03 | 1.76E−03 | 0.43 | 2.46 |
| Nuclei_RadialDistribution_RadialCV_Lipid_2of4 | 2.64E−04 | 4.89E−04 | 0.85 | −6.73 |
| Nuclei_RadialDistribution_RadialCV_Mito_3of4 | 2.16E−05 | 6.79E−05 | 0.82 | 5.99 |
| Nuclei_Texture_AngularSecondMoment_AGP_20_03 | 8.18E−03 | 7.55E−03 | 0.65 | −3.88 |
| Nuclei_Texture_AngularSecondMoment_DNA_20_03 | 2.57E−04 | 4.77E−04 | 0.52 | −2.93 |
| Nuclei_Texture_AngularSecondMoment_Lipid_20_02 | 2.51E−04 | 4.68E−04 | 0.84 | 6.37 |
| Nuclei_Texture_Contrast_Lipid_20_00 | 8.53E−07 | 1.40E−05 | 0.94 | −11.53 |
| Nuclei_Texture_Contrast_Mito_20_03 | 3.16E−04 | 5.64E−04 | 0.63 | −3.67 |
| Nuclei_Texture_Correlation_DNA_5_01 | 6.18E−03 | 6.02E−03 | 0.43 | 2.43 |
| Nuclei_Texture_DifferenceEntropy_DNA_20_01 | 2.75E−03 | 3.10E−03 | 0.44 | 2.51 |
| Nuclei_Texture_DifferenceEntropy_Lipid_20_02 | 3.55E−06 | 1.86E−05 | 0.95 | −11.78 |
| Nuclei_Texture_DifferenceEntropy_Mito_10_03 | 6.60E−03 | 6.35E−03 | 0.33 | −2.00 |
| Nuclei_Texture_DifferenceVariance_DNA_5_02 | 1.32E−03 | 1.72E−03 | 0.56 | −3.19 |
| Nuclei_Texture_DifferenceVariance_Lipid_20_02 | 1.69E−04 | 3.47E−04 | 0.81 | 5.88 |
| Nuclei_Texture_Entropy_AGP_20_02 | 3.59E−03 | 3.82E−03 | 0.70 | 4.36 |
| Nuclei_Texture_Entropy_DNA_5_00 | 1.11E−03 | 1.48E−03 | 0.54 | 3.05 |
| Nuclei_Texture_Entropy_Lipid_10_01 | 3.54E−05 | 1.00E−04 | 0.89 | −8.22 |
| Nuclei_Texture_Entropy_Mito_10_02 | 1.07E−02 | 9.33E−03 | 0.22 | −1.52 |
| Nuclei_Texture_InfoMeas1_DNA_10_00 | 4.46E−03 | 4.57E−03 | 0.59 | −3.40 |
| Nuclei_Texture_InfoMeas2_Lipid_20_00 | 5.21E−05 | 1.36E−04 | 0.89 | −8.03 |
| Nuclei_Texture_InfoMeas1_Mito_5_00 | 7.92E−04 | 1.13E−03 | 0.75 | 4.91 |
| Nuclei_Texture_InverseDifferenceMoment_DNA_20_01 | 2.73E−03 | 3.07E−03 | 0.59 | −3.41 |
| Nuclei_Texture_InverseDifferenceMoment_Lipid_10_03 | 3.23E−05 | 9.39E−05 | 0.85 | 6.81 |
| Nuclei_Texture_InverseDifferenceMoment_Mito_20_00 | 4.41E−03 | 4.52E−03 | 0.50 | 2.80 |
| Nuclei_Texture_SumAverage_Lipid_20_00 | 1.93E−07 | 1.07E−05 | 0.95 | −12.28 |
| Nuclei_Texture_SumAverage_Mito_10_03 | 2.09E−04 | 4.01E−04 | 0.77 | −5.18 |
| Nuclei_Texture_SumEntropy_AGP_20_01 | 8.37E−03 | 7.69E−03 | 0.65 | 3.87 |
| Nuclei_Texture_SumEntropy_DNA_10_01 | 9.27E−04 | 1.29E−03 | 0.52 | 2.93 |
| Nuclei_Texture_SumEntropy_Lipid_20_02 | 1.50E−05 | 5.10E−05 | 0.93 | −10.60 |
| Nuclei_Texture_SumEntropy_Mito_5_02 | 4.92E−03 | 4.96E−03 | 0.34 | −2.04 |
| Nuclei_Texture_SumVariance_Lipid_20_03 | 2.27E−06 | 1.55E−05 | 0.95 | −12.63 |
| Nuclei_Texture_SumVariance_Mito_10_03 | 5.97E−04 | 9.01E−04 | 0.62 | −3.57 |
| Nuclei_Texture_Variance_Lipid_20_00 | 9.23E−07 | 1.40E−05 | 0.95 | −12.53 |
| Nuclei_Texture_Variance_Mito_20_02 | 3.25E−04 | 5.75E−04 | 0.62 | −3.60 |

TABLE 20
Significant genes (padj <= 10e−6; log2 FC > |0.75|) of BCL2-KD-mediated gene
expression changes at day14 in subcutaneous adipocytes. DESeq analysis. EN number,
ensemble gene identification number; log2FC, fold change (log2); pvalue, p-value
(significance level p <= 0.05); padj, adjusted p-value

| log2FC | pvalue | padj | gene | gene name |
|---|---|---|---|---|
| −1.205300673 | 7.91E−13 | 3.13E−11 | FGR | FGR proto-oncogene, Src family tyrosine |
| | | | | kinase |
| −0.810889868 | 1.24E−08 | 2.14E−07 | BAD | BCL2 associated agonist of cell death |
| −0.804965103 | 1.43E−10 | 3.71E−09 | NDUFAB1 | NADH: ubiquinone oxidoreductase subunit |
| | | | | AB1 |
| −0.784875201 | 6.73E−11 | 1.83E−09 | ITGA3 | integrin subunit alpha 3 |
| −1.248995998 | 1.12E−11 | 3.62E−10 | REX1BD | required for excision 1-B domain containing |
| −0.882325298 | 3.29E−15 | 1.90E−13 | TRAPPC6A | trafficking protein particle complex 6A |
| −0.845463388 | 6.82E−09 | 1.26E−07 | PIGQ | phosphatidylinositol glycan anchor |
| | | | | biosynthesis class Q |
| −0.954819595 | 1.44E−09 | 3.05E−08 | PLEKHG6 | pleckstrin homology and RhoGEF domain |
| | | | | containing G6 |
| −1.15061344 | 8.90E−12 | 2.93E−10 | MAPK8IP2 | mitogen-activated protein kinase 8 |
| | | | | interacting protein 2 |
| −1.659254492 | 3.19E−08 | 5.11E−07 | GIPR | gastric inhibitory polypeptide receptor |
| −0.838757087 | 1.48E−10 | 3.83E−09 | NR1H3 | nuclear receptor subfamily 1 group H |
| | | | | member 3 |
| −1.001687136 | 1.54E−16 | 1.11E−14 | TYMP | thymidine phosphorylase |
| −0.783878565 | 3.01E−11 | 8.83E−10 | SLC66A1 | solute carrier family 66 member 1 |
| −0.944459625 | 3.47E−10 | 8.28E−09 | BCAR1 | BCAR1 scaffold protein, Cas family member |
| −0.79387441 | 1.75E−11 | 5.48E−10 | GYG2 | glycogenin 2 |
| −0.995022757 | 1.98E−11 | 6.07E−10 | OGFR | opioid growth factor receptor |
| −0.842797362 | 8.42E−09 | 1.53E−07 | TNK2 | tyrosine kinase non receptor 2 |
| −1.138473176 | 8.60E−19 | 8.64E−17 | ISOC2 | isochorismatase domain containing 2 |
| −0.809431305 | 5.17E−12 | 1.76E−10 | GPC1 | glypican 1 |
| −1.377649669 | 1.03E−22 | 1.70E−20 | ABCA7 | ATP binding cassette subfamily A member 7 |
| −0.815857595 | 1.16E−08 | 2.02E−07 | WDR18 | WD repeat domain 18 |
| −0.967458348 | 3.36E−12 | 1.19E−10 | FGFR2 | fibroblast growth factor receptor 2 |
| −1.124278706 | 7.34E−20 | 8.72E−18 | PLEKHH3 | pleckstrin homology, MyTH4 and FLipidM |
| | | | | domain containing H3 |
| −1.229251669 | 3.95E−07 | 4.96E−06 | ST6GALNAC2 | ST6 N-acetylgalactosaminide alpha-2,6- |
| | | | | sialyltransferase 2 |
| −0.992016077 | 5.00E−14 | 2.41E−12 | MBD3 | methyl-CpG binding domain protein 3 |
| −0.75336364 | 1.98E−12 | 7.29E−11 | SELENOO | selenoprotein O |
| −0.808199305 | 9.42E−08 | 1.37E−06 | ANO8 | anoctamin 8 |
| −1.588619372 | 4.10E−12 | 1.43E−10 | LIPE | lipase E, hormone sensitive type |
| −0.813134724 | 3.96E−10 | 9.36E−09 | COL5A3 | collagen type V alpha 3 chain |
| −0.971545202 | 1.78E−19 | 1.93E−17 | AKR1B1 | aldo-keto reductase family 1 member B |
| −1.173782514 | 2.20E−09 | 4.48E−08 | HSD17B14 | hydroxysteroid 17-beta dehydrogenase 14 |
| −0.99351616 | 1.77E−17 | 1.45E−15 | ACHE | acetylcholinesterase (Cartwright blood |
| | | | | group) |
| −0.884988019 | 4.04E−13 | 1.70E−11 | PEBP1 | phosphatidylethanolamine binding protein 1 |
| −0.84606865 | 1.92E−07 | 2.57E−06 | LTBP4 | latent transforming growth factor beta |
| | | | | binding protein 4 |
| −0.84316805 | 2.41E−11 | 7.20E−10 | BLVRB | biliverdin reductase B |
| −0.966509655 | 5.96E−09 | 1.11E−07 | ABCC6 | ATP binding cassette subfamily C member 6 |
| −0.866498556 | 6.89E−09 | 1.27E−07 | TYRO3 | TYRO3 protein tyrosine kinase |
| −1.702868695 | 6.90E−14 | 3.26E−12 | SH2D3C | SH2 domain containing 3C |
| −0.998358467 | 9.71E−12 | 3.16E−10 | NUBP2 | nucleotide binding protein 2 |
| −0.795315259 | 5.94E−07 | 7.24E−06 | SCD | stearoyl-CoA desaturase |
| −0.889362217 | 3.98E−10 | 9.40E−09 | ATP5F1D | ATP synthase F1 subunit delta |
| −1.051019478 | 3.96E−16 | 2.65E−14 | NDUFB7 | NADH: ubiquinone oxidoreductase |
| | | | | subunit B7 |
| −0.997765058 | 1.82E−12 | 6.76E−11 | CEP170B | centrosomal protein 170B |
| −0.844285324 | 4.29E−20 | 5.26E−18 | MKNK2 | MAPK interacting serine/threonine kinase 2 |
| −0.849590025 | 1.80E−09 | 3.74E−08 | P2RX6 | purinergic receptor P2X 6 |
| −1.196371966 | 4.71E−15 | 2.63E−13 | DDTL | D-dopachrome tautomerase like |
| −1.196371966 | 4.71E−15 | 2.63E−13 | DDT | D-dopachrome tautomerase like |
| −1.196371966 | 4.71E−15 | 2.63E−13 | DDT | D-dopachrome tautomerase |
| −0.963154224 | 1.06E−20 | 1.38E−18 | SLC25A1 | solute carrier family 25 member 1 |
| −0.789432304 | 1.03E−13 | 4.68E−12 | MICALL1 | MICAL like 1 |
| −0.768853962 | 2.23E−10 | 5.47E−09 | LMF2 | lipase maturation factor 2 |
| −0.840309529 | 6.35E−08 | 9.58E−07 | RHBDD3 | rhomboid domain containing 3 |
| −0.7669685 | 7.58E−10 | 1.69E−08 | ARSA | arylsulfatase A |
| −1.435095151 | 2.52E−31 | 1.15E−28 | TSPO | translocator protein |
| −1.744235951 | 2.58E−08 | 4.21E−07 | EEF1A2 | eukaryotic translation elongation factor 1 |
| | | | | alpha 2 |
| −0.755383172 | 3.69E−13 | 1.56E−11 | HSF4 | heat shock transcription factor 4 |
| −0.984212142 | 6.46E−09 | 1.20E−07 | NME3 | NME/NM23 nucleoside diphosphate kinase 3 |
| −1.004866892 | 6.25E−07 | 7.58E−06 | CAPN15 | calpain 15 |
| −0.819672987 | 2.19E−09 | 4.45E−08 | PIEZO1 | piezo type mechanosensitive ion channel |
| | | | | component 1 |
| −0.851613477 | 2.37E−22 | 3.76E−20 | QPRT | quinolinate phosphoribosyltransferase |
| −0.825460797 | 2.68E−07 | 3.50E−06 | BBC3 | BCL2 binding component 3 |
| −1.371572117 | 1.22E−20 | 1.58E−18 | MYH14 | myosin heavy chain 14 |
| −1.16424613 | 6.38E−18 | 5.71E−16 | LFNG | LFNG O-fucosylpeptide 3-beta-N- |
| | | | | acetylglucosaminyltransferase |
| −0.825581846 | 1.10E−09 | 2.39E−08 | BRAT1 | BRCA1 associated ATM activator 1 |
| −0.766421108 | 4.17E−12 | 1.45E−10 | CHCHD2 | coiled-coil-helix-coiled-coil-helix domain |
| | | | | containing 2 |
| −1.025538798 | 1.94E−15 | 1.16E−13 | HOXB6 | homeobox B6 |
| −0.969439663 | 3.69E−12 | 1.29E−10 | SYNGR2 | synaptogyrin 2 |
| −1.046403471 | 7.26E−15 | 3.97E−13 | ALDOC | aldolase, fructose-bisphosphate C |
| −1.709649773 | 1.79E−13 | 7.82E−12 | NRXN2 | neurexin 2 |
| −0.848057765 | 3.20E−17 | 2.57E−15 | AMBRA1 | autophagy and beclin 1 regulator 1 |
| −1.01347786 | 2.12E−09 | 4.32E−08 | SLC22A18 | solute carrier family 22 member 18 |
| −0.930531285 | 3.09E−12 | 1.10E−10 | NDUFS8 | NADH: ubiquinone oxidoreductase core |
| | | | | subunit S8 |
| −0.761341491 | 2.57E−14 | 1.29E−12 | MVK | mevalonate kinase |
| −0.833013044 | 1.86E−11 | 5.77E−10 | FANCE | FA complementation group E |
| −1.004472712 | 2.17E−10 | 5.33E−09 | TFEB | transcription factor EB |
| −0.80987096 | 3.83E−07 | 4.81E−06 | DNPH1 | 2′-deoxynucleoside 5′-phosphate |
| | | | | N-hydrolase 1 |
| −1.287303706 | 5.38E−09 | 1.02E−07 | REEP6 | receptor accessory protein 6 |
| −1.56856113 | 1.74E−19 | 1.90E−17 | NDUFS7 | NADH:ubiquinone oxidoreductase core |
| | | | | subunit S7 |
| −0.85967414 | 1.98E−11 | 6.07E−10 | PECR | peroxisomal trans-2-enoyl-CoA reductase |
| −0.825992543 | 3.97E−28 | 1.17E−25 | CTSD | cathepsin D |
| −1.464679912 | 6.89E−12 | 2.28E−10 | MMP8 | matrix metallopeptidase 8 |
| −0.778806882 | 9.57E−09 | 1.71E−07 | STBD1 | starch binding domain 1 |
| −0.909147157 | 2.84E−13 | 1.21E−11 | PTK2B | protein tyrosine kinase 2 beta |
| −1.448982945 | 3.17E−17 | 2.57E−15 | ADGRB2 | adhesion G protein-coupled receptor B2 |
| −1.464177804 | 2.12E−18 | 2.06E−16 | CNTFR | ciliary neurotrophic factor receptor |
| −0.993429875 | 9.40E−13 | 3.69E−11 | ACADS | acyl-CoA dehydrogenase short chain |
| −0.864898316 | 3.83E−19 | 3.95E−17 | CDKN2C | cyclin dependent kinase inhibitor 2C |
| −0.842285296 | 1.18E−10 | 3.10E−09 | PDE1B | phosphodiesterase 1B |
| −1.252800403 | 6.85E−07 | 8.23E−06 | SARDH | sarcosine dehydrogenase |
| −1.810247219 | 9.20E−16 | 5.69E−14 | GOS2 | GO/G1 switch 2 |
| −0.916994361 | 5.91E−12 | 1.98E−10 | PREX1 | phosphatidylinositol-3,4,5-trisphosphate |
| | | | | dependent Rac exchange factor 1 |
| −1.035558134 | 4.93E−08 | 7.61E−07 | SNAI1 | snail family transcriptional repressor 1 |
| −0.834491272 | 3.59E−15 | 2.05E−13 | PEPD | peptidase D |
| −1.093905551 | 1.63E−08 | 2.76E−07 | KLHL31 | kelch like family member 31 |
| −1.478937362 | 1.93E−08 | 3.23E−07 | SH3TC1 | SH3 domain and tetratricopeptide repeats 1 |
| −0.960700017 | 1.23E−07 | 1.73E−06 | SOX9 | SRY-box transcription factor 9 |
| −1.027567236 | 1.15E−07 | 1.62E−06 | ALKBH7 | alkB homolog 7 |
| −1.86261909 | 9.38E−10 | 2.06E−08 | CITED1 | Cbp/p300 interacting transactivator with |
| | | | | Glu/Asp rich carboxy-terminal domain 1 |
| −0.926724082 | 1.12E−12 | 4.30E−11 | TMEM53 | transmembrane protein 53 |
| −0.836534007 | 1.39E−17 | 1.17E−15 | NR1D1 | nuclear receptor subfamily 1 group D |
| | | | | member 1 |
| −0.801506076 | 9.32E−16 | 5.73E−14 | FRMD8 | FLipidM domain containing 8 |
| −0.778221205 | 1.97E−09 | 4.06E−08 | AHDC1 | AT-hook DNA binding motif containing 1 |
| −1.027551641 | 5.70E−13 | 2.31E−11 | IF16 | interferon alpha inducible protein 6 |
| −0.905096836 | 5.72E−12 | 1.93E−10 | PIN1 | peptidylprolyl cis/trans isomerase, NIMA- |
| | | | | interacting 1 |
| −0.871958541 | 2.90E−08 | 4.67E−07 | EMC6 | Lipid membrane protein complex subunit 6 |
| −1.196079966 | 5.85E−08 | 8.92E−07 | MGAT3 | beta-1,4-mannosyl-glycoprotein 4-beta-N- |
| | | | | acetylglucosaminyltransferase |
| −0.882440014 | 2.67E−16 | 1.82E−14 | MPST | mercaptopyruvate sulfurtransferase |
| −1.008748317 | 7.68E−14 | 3.57E−12 | TST | thiosulfate sulfurtransferase |
| −1.016449946 | 3.51E−09 | 6.82E−08 | ABHD17A | abhydrolase domain containing 17A, |
| | | | | depalmitoylase |
| −1.127640384 | 4.35E−11 | 1.24E−09 | ANGPTL8 | angiopoietin like 8 |
| −0.998840284 | 1.28E−12 | 4.86E−11 | THEM6 | thioesterase superfamily member 6 |
| −1.50641304 | 1.85E−27 | 5.15E−25 | APOE | apolipoprotein E |
| −1.090597524 | 1.71E−08 | 2.90E−07 | GADD45G | growth arrest and DNA damage inducible |
| | | | | gamma |
| −0.804171043 | 9.59E−20 | 1.12E−17 | SLC27A1 | solute carrier family 27 member 1 |
| −1.190500446 | 5.58E−18 | 5.08E−16 | MRPL34 | mitochondrial ribosomal protein L34 |
| −1.281295643 | 8.57E−09 | 1.55E−07 | FCHO1 | FCH and mu domain containing endocytic |
| | | | | adaptor 1 |
| −1.352953988 | 2.16E−10 | 5.32E−09 | LAMA5 | laminin subunit alpha 5 |
| −0.767057573 | 6.47E−17 | 4.96E−15 | ADRM1 | adhesion regulating molecule 1 |
| −0.787751938 | 3.91E−07 | 4.91E−06 | HIP1R | huntingtin interacting protein 1 related |
| −0.922043991 | 5.33E−12 | 1.81E−10 | ACSS2 | acyl-CoA synthetase short chain family |
| | | | | member 2 |
| −0.81873838 | 5.15E−11 | 1.45E−09 | ACAP3 | ArfGAP with coiled-coil, ankyrin repeat and |
| | | | | PH domains 3 |
| −0.821522709 | 1.98E−10 | 4.92E−09 | CKMT2 | creatine kinase, mitochondrial 2 |
| −0.951689538 | 1.04E−12 | 4.01E−11 | PPP1R1B | protein phosphatase 1 regulatory inhibitor |
| | | | | subunit 1B |
| −1.050279667 | 8.69E−09 | 1.57E−07 | RAP1GAP2 | RAP1 GTPase activating protein 2 |
| −0.956757908 | 2.78E−18 | 2.65E−16 | CLUH | clustered mitochondria homolog |
| −0.75897002 | 2.69E−07 | 3.52E−06 | RBM38 | RNA binding motif protein 38 |
| −1.228116374 | 5.58E−09 | 1.05E−07 | MACROD1 | mono-ADP ribosylhydrolase 1 |
| −1.0662622 | 5.78E−16 | 3.72E−14 | LGALS12 | galectin 12 |
| −1.024120465 | 2.10E−20 | 2.66E−18 | BHLHE40 | basic helix-loop-helix family member e40 |
| −0.756138157 | 1.12E−10 | 2.95E−09 | LPIN1 | lipin 1 |
| −0.846789004 | 2.26E−09 | 4.57E−08 | HAVCR2 | hepatitis A virus cellular receptor 2 |
| −0.976954636 | 2.79E−18 | 2.65E−16 | DTX1 | deltex E3 ubiquitin ligase 1 |
| −0.893690372 | 1.95E−16 | 1.36E−14 | ITGA7 | integrin subunit alpha 7 |
| −0.798028546 | 1.12E−18 | 1.12E−16 | RDH5 | retinol dehydrogenase 5 |
| −0.892112753 | 2.11E−12 | 7.74E−11 | PPP1R1A | protein phosphatase 1 regulatory inhibitor |
| | | | | subunit 1A |
| −1.01338538 | 1.74E−11 | 5.44E−10 | FAIM2 | Fas apoptotic inhibitory molecule 2 |
| −0.814805594 | 1.64E−09 | 3.45E−08 | COX5B | cytochrome c oxidase subunit 58 |
| −1.142942175 | 4.39E−13 | 1.82E−11 | VILL | villin like |
| −0.757804867 | 8.98E−15 | 4.82E−13 | ST6GALNAC4 | ST6 N-acetylgalactosaminide alpha-2,6- |
| | | | | sialyltransferase 4 |
| −0.79232329 | 2.55E−15 | 1.50E−13 | HINT2 | histidine triad nucleotide binding protein 2 |
| −0.874695414 | 4.69E−12 | 1.61E−10 | FOXP4 | forkhead box P4 |
| −0.753841506 | 3.95E−11 | 1.14E−09 | TUBB2B | tubulin beta 2B class IIb |
| −0.951784556 | 3.59E−15 | 2.05E−13 | RBP4 | retinol binding protein 4 |
| −0.855382463 | 1.95E−14 | 9.95E−13 | DUSP6 | dual specificity phosphatase 6 |
| −0.846494221 | 3.65E−07 | 4.63E−06 | NEIL1 | nei like DNA glycosylase 1 |
| −1.477990212 | 2.00E−09 | 4.11E−08 | ADAMTS18 | ADAM metallopeptidase with |
| | | | | thrombospondin type 1 motif 18 |
| −0.894593984 | 1.22E−14 | 6.42E−13 | NOL3 | nucleolar protein 3 |
| −0.763388559 | 2.61E−11 | 7.75E−10 | RHOT2 | ras homolog family member T2 |
| −0.899278416 | 1.73E−19 | 1.89E−17 | SCRN2 | secernin 2 |
| −1.449802859 | 6.60E−07 | 7.97E−06 | TMC6 | transmembrane channel like 6 |
| −0.995921944 | 4.77E−08 | 7.37E−07 | RNF157 | ring finger protein 157 |
| −2.158000704 | 1.36E−09 | 2.88E−08 | TPGS1 | tubulin polyglutamylase complex subunit 1 |
| −0.789399011 | 1.28E−16 | 9.27E−15 | RNPEPL1 | arginyl aminopeptidase like 1 |
| −0.97027802 | 5.37E−11 | 1.50E−09 | SLC47A1 | solute carrier family 47 member 1 |
| −1.182332376 | 3.91E−20 | 4.87E−18 | MPC2 | mitochondrial pyruvate carrier 2 |
| −0.850511281 | 2.68E−11 | 7.96E−10 | EBP | EBP cholestenol delta-isomerase |
| −0.754425794 | 1.13E−08 | 1.97E−07 | NAPRT | nicotinate phosphoribosyltransferase |
| −0.79432051 | 8.20E−15 | 4.43E−13 | PLIN2 | perilipin 2 |
| −0.993470372 | 5.37E−26 | 1.29E−23 | TM7SF2 | transmembrane 7 superfamily member 2 |
| −1.074340885 | 4.20E−09 | 8.09E−08 | ABCB9 | ATP binding cassette subfamily B member 9 |
| −1.033756524 | 6.22E−16 | 3.96E−14 | THRSP | thyroid hormone responsive |
| −0.91678656 | 2.91E−09 | 5.73E−08 | DPYSL4 | dihydropyrimidinase like 4 |
| −1.51931394 | 2.05E−10 | 5.08E−09 | MZT2B | mitotic spindle organizing protein 2B |
| −0.939322807 | 2.08E−07 | 2.76E−06 | UBALD1 | UBA like domain containing 1 |
| −0.796825598 | 3.00E−11 | 8.82E−10 | C16orf89 | chromosome 16 open reading frame 89 |
| −0.99386595 | 3.39E−08 | 5.40E−07 | OBSCN | obscurin, cytoskeletal calmodulin and titin- |
| | | | | interacting RhoGEF |
| −1.055557156 | 3.08E−14 | 1.53E−12 | TIMP4 | TIMP metallopeptidase inhibitor 4 |
| −1.007195111 | 3.75E−07 | 4.72E−06 | RNF207 | ring finger protein 207 |
| −0.815866296 | 6.58E−12 | 2.19E−10 | FBXW5 | F-box and WD repeat domain containing 5 |
| −1.186225172 | 3.20E−19 | 3.39E−17 | SV2A | synaptic vesicle glycoprotein 2A |
| −0.913292707 | 7.90E−11 | 2.13E−09 | HK2 | hexokinase 2 |
| −1.710733925 | 1.17E−12 | 4.47E−11 | CCDC107 | coiled-coil domain containing 107 |
| −0.843405009 | 3.91E−11 | 1.13E−09 | CFAP410 | cilia and flagella associated protein 410 |
| −1.040831084 | 2.31E−09 | 4.65E−08 | FAM207A | family with sequence similarity 207 |
| | | | | member A |
| −0.891188651 | 1.47E−24 | 3.00E−22 | LSS | lanosterol synthase |
| −1.691743117 | 3.49E−07 | 4.45E−06 | SLC2A6 | solute carrier family 2 member 6 |
| −0.916675774 | 1.65E−07 | 2.26E−06 | PTH1R | parathyroid hormone 1 receptor |
| −1.050965395 | 1.33E−12 | 5.04E−11 | DMKN | dermokine |
| −1.401948353 | 5.88E−51 | 2.29E−47 | CYGB | cytoglobin |
| −1.02868646 | 5.24E−11 | 1.47E−09 | ALDH16A1 | aldehyde dehydrogenase 16 family |
| | | | | member A1 |
| −1.006906146 | 2.30E−28 | 7.31E−26 | ZNF385A | zinc finger protein 385A |
| −1.091791253 | 3.59E−11 | 1.04E−09 | JOSD2 | Josephin domain containing 2 |
| −0.822325123 | 2.40E−11 | 7.20E−10 | SPSB3 | splA/ryanodine receptor domain and SOCS |
| | | | | box containing 3 |
| −0.975813507 | 5.37E−11 | 1.50E−09 | AMDHD2 | amidohydrolase domain containing 2 |
| −0.860166833 | 2.44E−12 | 8.79E−11 | DHRS3 | dehydrogenase/reductase 3 |
| −1.022677115 | 2.65E−09 | 5.28E−08 | FAAP20 | FA core complex associated protein 20 |
| −0.890662061 | 6.84E−16 | 4.28E−14 | IGSF8 | immunoglobulin superfamily member 8 |
| −1.087636414 | 4.73E−08 | 7.33E−07 | FCRLB | Fc receptor like B |
| −0.763135459 | 5.59E−07 | 6.83E−06 | C1orf115 | chromosome 1 open reading frame 115 |
| −0.861167932 | 1.19E−11 | 3.83E−10 | KIF26B | kinesin family member 26B |
| −1.003128603 | 2.43E−11 | 7.25E−10 | ADORA1 | adenosine A1 receptor |
| −1.277873049 | 6.43E−12 | 2.14E−10 | CCR1 | C—C motif chemokine receptor 1 |
| −1.028680786 | 1.57E−08 | 2.67E−07 | ACSL6 | acyl-CoA synthetase long chain family |
| | | | | member 6 |
| −1.133102068 | 9.64E−17 | 7.15E−15 | SLC29A4 | solute carrier family 29 member 4 |
| −1.339165101 | 7.07E−10 | 1.60E−08 | GPR146 | G protein-coupled receptor 146 |
| −1.413681604 | 3.78E−28 | 1.14E−25 | GPLipid1 | G protein-coupled estrogen receptor 1 |
| −1.279741555 | 5.06E−07 | 6.23E−06 | NOS3 | nitric oxide synthase 3 |
| −0.763023587 | 8.66E−11 | 2.31E−09 | INTS1 | integrator complex subunit 1 |
| −1.246853055 | 3.42E−22 | 5.22E−20 | MID1IP1 | MID1 interacting protein 1 |
| −0.812609198 | 2.76E−07 | 3.59E−06 | AQP3 | aquaporin 3 (Gill blood group) |
| −0.85400866 | 3.01E−09 | 5.92E−08 | ZNF219 | zinc finger protein 219 |
| −0.972636208 | 1.62E−16 | 1.16E−14 | CKB | creatine kinase B |
| −1.624954245 | 9.45E−10 | 2.07E−08 | TBATA | thymus, brain and testes associated |
| −1.244610095 | 7.49E−16 | 4.65E−14 | LDHD | lactate dehydrogenase D |
| −0.783603547 | 9.41E−10 | 2.06E−08 | ACSF2 | acyl-CoA synthetase family member 2 |
| −1.505808883 | 5.83E−10 | 1.34E−08 | MVD | mevalonate diphosphate decarboxylase |
| −0.805890578 | 3.02E−11 | 8.84E−10 | TP53113 | tumor protein p53 inducible protein 13 |
| −0.751042061 | 4.60E−08 | 7.15E−07 | RAB4B | RAB4B, member RAS oncogene family |
| −0.813281635 | 5.13E−11 | 1.45E−09 | GPD1 | glycerol-3-phosphate dehydrogenase 1 |
| −0.791249472 | 1.68E−16 | 1.19E−14 | UBXN6 | UBX domain protein 6 |
| −1.24543338 | 2.73E−16 | 1.85E−14 | PLIN4 | perilipin 4 |
| −0.98972375 | 2.00E−08 | 3.34E−07 | MFSD3 | major facilitator superfamily domain |
| | | | | containing 3 |
| −1.072094708 | 8.00E−09 | 1.46E−07 | KIFC2 | kinesin family member C2 |
| −0.858942758 | 3.21E−11 | 9.36E−10 | RILP | Rab interacting lysosomal protein |
| −1.182467069 | 2.10E−16 | 1.46E−14 | RCOR2 | REST corepressor 2 |
| −0.918643467 | 8.85E−11 | 2.36E−09 | E4F1 | E4F transcription factor 1 |
| −0.756405075 | 2.68E−08 | 4.36E−07 | CAVIN2 | caveolae associated protein 2 |
| −0.803434807 | 1.01E−12 | 3.94E−11 | SLipidINC2 | serine incorporator 2 |
| −0.88160048 | 4.32E−12 | 1.50E−10 | LGALS9 | galectin 9 |
| −0.901825429 | 1.62E−13 | 7.13E−12 | SLC49A3 | solute carrier family 49 member 3 |
| −0.750244936 | 4.67E−26 | 1.14E−23 | NPR1.00 | natriuretic peptide receptor 1 |
| −1.300385933 | 3.26E−08 | 5.22E−07 | AGPAT2 | 1-acylglycerol-3-phosphate |
| | | | | O-acyltransferase2 |
| −1.679202704 | 3.54E−14 | 1.75E−12 | FASN | fatty acid synthase |
| −0.83198878 | 6.09E−16 | 3.89E−14 | DCXR | dicarbonyl and L-xylulose reductase |
| −1.121025165 | 2.31E−12 | 8.42E−11 | RAC3 | Rac family small GTPase 3 |
| −0.871210422 | 2.37E−13 | 1.03E−11 | MRAP | melanocortin 2 receptor accessory protein |
| −1.134809097 | 1.81E−07 | 2.44E−06 | GPRC5C | G protein-coupled receptor class C group 5 |
| | | | | member C |
| −1.70514406 | 1.01E−50 | 3.16E−47 | CD14 | CD14 molecule |
| −2.088527131 | 1.80E−10 | 4.50E−09 | HSD17B13 | hydroxysteroid 17-beta dehydrogenase 13 |
| −0.813002253 | 1.26E−07 | 1.76E−06 | TRABD | TraB domain containing |
| −0.853590812 | 3.00E−13 | 1.28E−11 | CAVIN3 | caveolae associated protein 3 |
| −1.336603657 | 1.50E−25 | 3.49E−23 | C9orf16 | chromosome 9 open reading frame 16 |
| −0.986398468 | 4.95E−13 | 2.04E−11 | SCAND1 | SCAN domain containing 1 |
| −1.049027985 | 4.72E−18 | 4.35E−16 | CXXC5 | CXXC finger protein 5 |
| −1.435216938 | 1.22E−11 | 3.92E−10 | BCL2 | BCL2 apoptosis regulator |
| −1.178759438 | 1.32E−10 | 3.46E−09 | PWWP2B | PWWP domain containing 2B |
| −1.942370036 | 6.23E−08 | 9.43E−07 | CYP4F22 | cytochrome P450 family 4 subfamily F |
| | | | | member 22 |
| −1.085036597 | 1.89E−12 | 6.99E−11 | CALB2 | calbindin 2 |
| −1.22315191 | 1.72E−12 | 6.42E−11 | MCRIP2 | MAPK regulated corepressor interacting |
| | | | | protein 2 |
| −1.140025202 | 8.67E−09 | 1.57E−07 | EGFL7 | EGF like domain multiple 7 |
| −0.858937854 | 4.02E−17 | 3.18E−15 | DHCR7 | 7-dehydrocholesterol reductase |
| −0.813198502 | 1.90E−08 | 3.19E−07 | ADCK5 | aarF domain containing kinase 5 |
| −0.785699048 | 4.32E−10 | 1.02E−08 | MRPL57 | mitochondrial ribosomal protein L57 |
| −0.908642099 | 3.20E−15 | 1.86E−13 | ESRRA | estrogen related receptor alpha |
| −1.636415606 | 7.85E−09 | 1.43E−07 | SNCG | synuclein gamma |
| −1.032566651 | 1.00E−20 | 1.32E−18 | PC | pyruvate carboxylase |
| −1.083669423 | 4.68E−08 | 7.26E−07 | LRFN4 | leucine rich repeat and fibronectin type III |
| | | | | domain containing 4 |
| −1.321251438 | 1.58E−14 | 8.19E−13 | SLC19A1 | solute carrier family 19 member 1 |
| −0.926859496 | 2.10E−09 | 4.29E−08 | JUP | junction plakoglobin |
| −0.773165381 | 6.94E−22 | 1.05E−19 | C1QTNF1 | C1q and TNF related 1 |
| −0.758337467 | 2.79E−09 | 5.53E−08 | MED16 | mediator complex subunit 16 |
| −1.041241451 | 2.96E−22 | 4.57E−20 | UCP2 | uncoupling protein 2 |
| −1.74389392 | 2.01E−11 | 6.15E−10 | CCDC85B | coiled-coil domain containing 85B |
| −0.757530616 | 1.20E−12 | 4.60E−11 | AURKAIP1 | aurora kinase A interacting protein 1 |
| −0.771837752 | 1.77E−16 | 1.24E−14 | PLAAT3 | phospholipase A and acyltransferase 3 |
| −1.083063349 | 1.37E−12 | 5.15E−11 | PIDD1 | p53-induced death domain protein 1 |
| −1.115530651 | 4.40E−20 | 5.36E−18 | PNPLA2 | patatin like phospholipase domain |
| | | | | containing 2 |
| −1.096792087 | 1.55E−07 | 2.12E−06 | MAMDC4 | MAM domain containing 4 |
| −1.036128855 | 3.61E−12 | 1.27E−10 | BOLA1 | bolA family member 1 |
| −1.254751577 | 4.89E−23 | 8.46E−21 | OPLAH | 5-oxoprolinase, ATP-hydrolysing |
| −0.960635689 | 1.48E−08 | 2.53E−07 | EXOSC4 | exosome component 4 |
| −1.431865381 | 4.55E−11 | 1.29E−09 | VWA1 | von Willebrand factor A domain containing 1 |
| −0.786866562 | 4.48E−18 | 4.15E−16 | LYNX1 | Ly6/neurotoxin 1 |
| −0.783856804 | 4.12E−07 | 5.15E−06 | H2AC6 | H2A clustered histone 6 |
| −0.85009246 | 3.13E−08 | 5.03E−07 | SCRIB | scribble planar cell polarity protein |
| −1.257053825 | 2.25E−15 | 1.33E−13 | TMEM132C | transmembrane protein 132C |
| −1.390086168 | 5.70E−13 | 2.31E−11 | PHLDA2 | pleckstrin homology like domain family A |
| | | | | member 2 |
| −0.79895785 | 1.69E−11 | 5.33E−10 | TMEM259 | transmembrane protein 259 |
| −0.917270031 | 1.78E−10 | 4.47E−09 | MRPL41 | mitochondrial ribosomal protein L41 |
| −1.051413309 | 2.08E−09 | 4.25E−08 | SLC25A18 | solute carrier family 25 member 18 |
| −1.136652164 | 3.54E−17 | 2.82E−15 | SLC25A10 | solute carrier family 25 member 10 |
| −0.788920487 | 8.02E−12 | 2.65E−10 | UPP1 | uridine phosphorylase 1 |
| −1.430200881 | 3.98E−20 | 4.92E−18 | NUDT14 | nudix hydrolase 14 |
| −0.761513169 | 7.23E−10 | 1.62E−08 | TXNRD2 | thioredoxin reductase 2 |
| −1.054832466 | 9.38E−08 | 1.36E−06 | PTP4A3 | protein tyrosine phosphatase 4A3 |
| −1.212826029 | 3.13E−21 | 4.56E−19 | C14orf180 | chromosome 14 open reading frame 180 |
| −1.793727734 | 4.57E−16 | 3.01E−14 | JAG2 | jagged canonical Notch ligand 2 |
| −0.767435832 | 4.22E−08 | 6.61E−07 | PTRHD1 | peptidyl-tRNA hydrolase domain |
| | | | | containing 1 |
| −0.785642761 | 1.73E−07 | 2.34E−06 | GAS2L1 | growth arrest specific 2 like 1 |
| −0.90913044 | 6.40E−24 | 1.20E−21 | PCYT2 | phosphate cytidylyltransferase 2, |
| | | | | ethanolamine |
| −0.884780213 | 5.18E−11 | 1.45E−09 | ATP6VOC | ATPase H+ transporting VO subunit c |
| −0.827036253 | 1.70E−28 | 5.51E−26 | KLHDC8B | kelch domain containing 8B |
| −0.974615478 | 7.26E−11 | 1.96E−09 | CYP4F12 | cytochrome P450 family 4 subfamily F |
| | | | | member 12 |
| −0.826595505 | 6.49E−10 | 1.48E−08 | RPS19BP1 | ribosomal protein S19 binding protein 1 |
| −0.966715412 | 1.23E−11 | 3.92E−10 | CMC1 | C-X9-C motif containing 1 |
| −0.951031337 | 1.91E−14 | 9.78E−13 | CIDEC | cell death inducing DFFA like effector c |
| −1.026537517 | 1.38E−09 | 2.92E−08 | AGRN | agrin |
| −0.815923595 | 1.61E−15 | 9.76E−14 | FAM53B | family with sequence similarity 53 member B |
| −0.791846233 | 1.62E−10 | 4.12E−09 | VKORC1L1 | vitamin K epoxide reductase complex |
| | | | | subunit 1 like 1 |
| −0.885189629 | 2.95E−08 | 4.75E−07 | COL27A1 | collagen type XXVII alpha 1 chain |
| −1.08717997 | 3.10E−13 | 1.31E−11 | LAMB3 | laminin subunit beta 3 |
| −1.027230009 | 6.60E−08 | 9.90E−07 | MIB2 | mindbomb E3 ubiquitin protein ligase 2 |
| −0.956003808 | 7.99E−15 | 4.34E−13 | RAB40C | RAB40C, member RAS oncogene family |
| −1.096813169 | 4.70E−16 | 3.09E−14 | CFD | complement factor D |
| −0.923333571 | 1.71E−12 | 6.40E−11 | PIM3 | Pim-3 proto-oncogene, serine/threonine |
| | | | | kinase |
| −0.753313522 | 2.35E−14 | 1.19E−12 | ANKRD35 | ankyrin repeat domain 35 |
| −1.364305928 | 6.72E−19 | 6.89E−17 | MMP17 | matrix metallopeptidase 17 |
| −0.795658406 | 7.43E−14 | 3.48E−12 | CCDC69 | coiled-coil domain containing 69 |
| −0.911138264 | 2.48E−09 | 4.95E−08 | SELENOM | selenoprotein M |
| −0.755232499 | 3.84E−17 | 3.05E−15 | CES1 | carboxylesterase 1 |
| −0.801389084 | 4.68E−12 | 1.61E−10 | INF2 | inverted formin 2 |
| −1.553945714 | 2.66E−14 | 1.33E−12 | H2AC18 | H2A clustered histone 18 |
| −0.846347603 | 2.80E−21 | 4.15E−19 | HSPA1B | heat shock protein family A (Hsp70) |
| | | | | member 1B |
| −0.79774136 | 1.14E−19 | 1.32E−17 | HSPA1A | heat shock protein family A (Hsp70) |
| | | | | member 1A |
| −0.851439682 | 7.35E−19 | 7.43E−17 | HLA-F | major histocompatibility complex, class I, F |
| −1.964831693 | 1.02E−09 | 2.23E−08 | UQCC3 | ubiquinol-cytochrome c reductase complex |
| | | | | assembly factor 3 |
| −2.640514366 | 2.19E−08 | 3.63E−07 | ADGRG1 | adhesion G protein-coupled receptor G1 |
| −0.944358299 | 1.26E−09 | 2.70E−08 | HCP5 | HLA complex P5 |
| −0.932570033 | 2.05E−08 | 3.41E−07 | SELENOH | selenoprotein H |
| −0.910631731 | 2.39E−09 | 4.80E−08 | C8orf82 | chromosome 8 open reading frame 82 |
| −0.879589039 | 8.60E−10 | 1.90E−08 | CSKMT | citrate synthase lysine methyltransferase |
| −1.007128445 | 7.21E−08 | 1.07E−06 | PAM16 | presequence translocase associated |
| | | | | motor1 6 |
| −1.807900286 | 1.37E−08 | 2.35E−07 | C6orf226 | chromosome 6 open reading frame 226 |
| −0.937264778 | 1.85E−09 | 3.83E−08 | OLMALINC | oligodendrocyte maturation-associated long |
| | | | | intergenic non-coding RNA |
| −0.972912643 | 2.59E−12 | 9.29E−11 | MIF | macrophage migration inhibitory factor |
| −0.873186533 | 2.99E−12 | 1.07E−10 | MTFP1 | mitochondrial fission process 1 |
| −0.817896563 | 8.57E−11 | 2.29E−09 | PEG10 | paternally expressed 10 |
| −0.993137221 | 1.72E−10 | 4.34E−09 | DECR2 | 2,4-dienoyl-CoA reductase 2 |
| −0.88528626 | 9.05E−14 | 4.14E−12 | AP5Z1 | adaptor related protein complex 5 subunit |
| | | | | zeta 1 |
| −0.850050324 | 2.50E−08 | 4.09E−07 | FAM86DP | family with sequence similarity 86 |
| | | | | member D, pseudogene |
| −0.986209966 | 1.94E−16 | 1.35E−14 | CEBPA | CCAAT enhancer binding protein alpha |
| −0.909292218 | 2.07E−18 | 2.03E−16 | H2AJ | H2A.J histone |
| −1.157889756 | 5.72E−24 | 1.10E−21 | TWF2 | twinfilin actin binding protein 2 |
| −0.775089674 | 1.43E−09 | 3.02E−08 | CHCHD10 | coiled-coil-helix-coiled-coil-helix domain |
| | | | | containing 10 |
| −1.127815874 | 5.59E−10 | 1.29E−08 | SHANK3 | SH3 and multiple ankyrin repeat domains 3 |
| −1.16756269 | 8.01E−07 | 9.52E−06 | LINC02749 | long intergenic non-protein coding RNA 2749 |
| −0.816532372 | 2.51E−08 | 4.11E−07 | CORO7 | coronin 7 |
| −0.857534102 | 1.04E−08 | 1.83E−07 | AGAP11 | ArfGAP with GTPase domain, ankyrin repeat |
| | | | | and PH domain 11 |
| −1.591352909 | 1.19E−14 | 6.28E−13 | H2AC19 | H2A clustered histone 19 |
| −1.261980676 | 3.51E−08 | 5.56E−07 | SRCIN1 | SRC kinase signaling inhibitor 1 |
| −1.110224944 | 4.38E−10 | 1.03E−08 | SOD2-OT1 | SOD2 overlapping transcript 1 |
| 0.867564367 | 3.96E−08 | 6.22E−07 | TMEM176A | transmembrane protein 176A |
| 0.773885381 | 3.83E−19 | 3.95E−17 | TFPI | tissue factor pathway inhibitor |
| 0.886126816 | 3.41E−07 | 4.36E−06 | FMO1 | flavin containing dimethylaniline |
| | | | | monoxygenase 1 |
| 0.91810421 | 1.87E−25 | 4.21E−23 | DCN | decorin |
| 0.858935463 | 8.58E−09 | 1.55E−07 | HGF | hepatocyte growth factor |
| 0.803807247 | 9.95E−20 | 1.16E−17 | RAI14 | retinoic acid induced 14 |
| 1.069136444 | 5.46E−14 | 2.62E−12 | LMO3 | LIM domain only 3 |
| 0.975972056 | 1.25E−24 | 2.59E−22 | KITLG | KIT ligand |
| 0.817851708 | 1.86E−11 | 5.77E−10 | ELN | elastin |
| 1.069633525 | 7.03E−25 | 1.48E−22 | SYNE2 | spectrin repeat containing nuclear envelope |
| | | | | protein 2 |
| 1.305445639 | 3.04E−43 | 4.73E−40 | COL11A1 | collagen type XI alpha 1 chain |
| 1.090046587 | 7.59E−14 | 3.54E−12 | GUCY1B1 | guanylate cyclase 1 soluble subunit beta 1 |
| 1.0802581 | 3.04E−29 | 1.03E−26 | LMCD1 | LIM and cysteine rich domains 1 |
| 1.1218649 | 5.91E−10 | 1.36E−08 | EDN1 | endothelin 1 |
| 0.797211173 | 5.27E−12 | 1.79E−10 | STK17B | serine/threonine kinase 17b |
| 0.883690413 | 1.65E−23 | 3.05E−21 | KCNK2 | potassium two pore domain channel |
| | | | | subfamily K member 2 |
| 0.92329765 | 2.22E−35 | 1.82E−32 | PTGS1 | prostaglandin-endoperoxide synthase 1 |
| 1.332400389 | 1.59E−13 | 7.08E−12 | DSP | desmoplakin |
| 0.97138283 | 9.84E−08 | 1.42E−06 | SUSD2 | sushi domain containing 2 |
| 1.378779261 | 8.53E−13 | 3.36E−11 | GGT5 | gamma-glutamyltransferase 5 |
| 1.461577572 | 8.01E−14 | 3.71E−12 | ISM1 | isthmin 1 |
| 1.085631438 | 4.78E−49 | 1.24E−45 | SAMHD1 | SAM and HD domain containing |
| | | | | deoxynucleoside triphosphate |
| | | | | triphosphohydrolase 1 |
| 0.816995752 | 3.33E−21 | 4.80E−19 | JAG1 | jagged canonical Notch ligand 1 |
| 0.982417511 | 2.01E−27 | 5.48E−25 | CRISPLD2 | cysteine rich secretory protein LCCL domain |
| | | | | containing 2 |
| 0.901762818 | 1.64E−10 | 4.15E−09 | CEMIP | cell migration inducing hyaluronidase 1 |
| 0.863327836 | 5.50E−15 | 3.05E−13 | PDGFRL | platelet derived growth factor receptor like |
| 0.754828073 | 1.53E−10 | 3.94E−09 | SFRP1 | secreted frizzled related protein 1 |
| 1.147558716 | 1.85E−09 | 3.83E−08 | IL7 | interleukin 7 |
| 2.555415803 | 2.73E−07 | 3.55E−06 | STMN2 | stathmin 2 |
| 0.870727992 | 2.23E−19 | 2.39E−17 | SLipidPINE1 | serpin family E member 1 |
| 1.136855934 | 7.38E−33 | 4.11E−30 | TMEM176B | transmembrane protein 176B |
| 0.956431006 | 7.49E−13 | 2.98E−11 | BST1 | bone marrow stromal cell antigen 1 |
| 1.118937862 | 1.03E−31 | 4.99E−29 | CTSC | cathepsin C |
| 0.868158603 | 9.88E−10 | 2.15E−08 | MGP | matrix Gla protein |
| 0.832909614 | 4.42E−16 | 2.94E−14 | SLC38A1 | solute carrier family 38 member 1 |
| 1.188271386 | 8.72E−38 | 9.05E−35 | COL12A1 | collagen type XII alpha 1 chain |
| 1.420529196 | 2.48E−34 | 1.84E−31 | SMOC2 | SPARC related modular calcium binding 2 |
| 1.068282103 | 1.13E−07 | 1.61E−06 | ENPP5 | ectonucleotide |
| | | | | pyrophosphatase/phosphodiesterase family |
| | | | | member 5 |
| 0.78499391 | 3.11E−08 | 5.00E−07 | SEMA5A | semaphorin 5A |
| 0.813149651 | 3.38E−36 | 3.10E−33 | LOX | lysyl oxidase |
| 1.451771611 | 3.27E−09 | 6.39E−08 | NPR 3.00 | natriuretic peptide receptor 3 |
| 0.917693512 | 1.41E−16 | 1.01E−14 | STC2 | stanniocalcin 2 |
| 0.7993153 | 3.10E−24 | 6.11E−22 | GNB4 | G protein subunit beta 4 |
| 0.765041883 | 9.87E−26 | 2.33E−23 | PLSCR4 | phospholipid scramblase 4 |
| 0.976758166 | 6.46E−18 | 5.75E−16 | PDE1A | phosphodiesterase 1A |
| 0.937501837 | 1.62E−33 | 1.09E−30 | EFEMP1 | EGF containing fibulin extracellular matrix |
| | | | | protein 1 |
| 1.408296436 | 4.82E−11 | 1.36E−09 | EFHD1 | EF-hand domain family member D1 |
| 0.814981104 | 1.90E−23 | 3.44E−21 | ANGPTL1 | angiopoietin like 1 |
| 0.86084403 | 1.01E−22 | 1.69E−20 | OLFML3 | olfactomedin like 3 |
| 1.081751017 | 3.02E−29 | 1.03E−26 | SGK1 | serum/glucocorticoid regulated kinase 1 |
| 1.018680646 | 6.43E−28 | 1.85E−25 | CCN2 | cellular communication network factor 2 |
| 0.764429051 | 3.52E−09 | 6.82E−08 | TGFB3 | transforming growth factor beta 3 |
| 1.584918206 | 6.09E−24 | 1.16E−21 | TEK | TEK receptor tyrosine kinase |
| 0.936830916 | 9.70E−15 | 5.19E−13 | MOB3B | MOB kinase activator 3B |
| 0.752392314 | 3.69E−08 | 5.82E−07 | PLXDC2 | plexin domain containing 2 |
| 0.940988996 | 4.05E−21 | 5.63E−19 | TNFSF10 | TNF superfamily member 10 |
| 0.792358443 | 1.65E−24 | 3.30E−22 | FMOD | fibromodulin |
| 0.875432905 | 6.36E−37 | 6.19E−34 | RECK | reversion inducing cysteine rich protein with |
| | | | | kazal motifs |
| 1.007115473 | 2.83E−34 | 2.00E−31 | BICC1 | BicC family RNA binding protein 1 |
| 2.115815954 | 4.64E−15 | 2.61E−13 | NRK | Nik related kinase |
| 0.85482601 | 6.69E−09 | 1.24E−07 | ABCC4 | ATP binding cassette subfamily C member 4 |
| 0.921802016 | 2.75E−22 | 4.32E−20 | AMOT | angiomotin |
| 1.016046433 | 5.93E−17 | 4.59E−15 | OMD | osteomodulin |
| 1.011983336 | 3.78E−26 | 9.50E−24 | MASP1 | mannan binding lectin serine peptidase 1 |
| 0.754151979 | 9.28E−08 | 1.35E−06 | STEAP4 | STEAP4 metalloreductase |
| 0.868558433 | 1.16E−48 | 2.57E−45 | CALU | calumenin |
| 0.845589436 | 3.94E−21 | 5.53E−19 | PALLD | palladin, cytoskeletal associated protein |
| 1.462649465 | 3.80E−28 | 1.14E−25 | GALNT15 | polypeptide N- |
| | | | | acetylgalactosaminyltransferase 15 |
| 1.058977928 | 3.68E−11 | 1.07E−09 | RAI2 | retinoic acid induced 2 |
| 0.851838179 | 1.11E−31 | 5.26E−29 | EMILIN2 | elastin microfibril interfacer 2 |
| 0.976629418 | 2.48E−08 | 4.07E−07 | LRRCC1 | leucine rich repeat and coiled-coil |
| | | | | centrosomal protein 1 |
| 0.779024231 | 1.02E−26 | 2.64E−24 | EMP1 | epithelial membrane protein 1 |
| 0.76714561 | 1.84E−11 | 5.73E−10 | TES | testin LIM domain protein |
| 0.81203427 | 1.71E−07 | 2.32E−06 | LCA5 | lebercilin LCA5 |
| 0.755317564 | 7.53E−11 | 2.03E−09 | LMO7 | LIM domain 7 |
| 1.358615469 | 3.28E−38 | 3.65E−35 | IL33 | interleukin 33 |
| 0.917209263 | 1.04E−10 | 2.75E−09 | SULF1 | sulfatase 1 |
| 1.005299391 | 4.98E−20 | 5.96E−18 | THBS1 | thrombospondin 1 |
| 0.806178271 | 2.28E−09 | 4.60E−08 | PRKG2 | protein kinase cGMP-dependent 2 |
| 0.808869183 | 4.46E−07 | 5.55E−06 | PDE5A | phosphodiesterase 5A |
| 1.246650205 | 3.33E−08 | 5.32E−07 | PTPRQ | protein tyrosine phosphatase receptor type Q |
| 1.01164151 | 1.85E−29 | 6.71E−27 | FBLN5 | fibulin 5 |
| 0.829870462 | 5.50E−27 | 1.45E−24 | FGF7 | fibroblast growth factor 7 |
| 0.779766326 | 2.61E−15 | 1.53E−13 | CDH11 | cadherin 11 |
| 1.025463033 | 2.68E−30 | 1.10E−27 | ABCA8 | ATP binding cassette subfamily A member 8 |
| 0.791770985 | 1.62E−24 | 3.27E−22 | CCN1 | cellular communication network factor 1 |
| 1.095833195 | 4.88E−08 | 7.53E−07 | ATP1B1 | ATPase Na+/K+ transporting subunit beta 1 |
| 1.006182148 | 8.83E−35 | 6.87E−32 | DPT | dermatopontin |
| 1.052365488 | 2.58E−08 | 4.21E−07 | STAC | SH3 and cysteine rich domain |
| 1.696055026 | 8.26E−07 | 9.79E−06 | COL8A1 | collagen type Vill alpha 1 chain |
| 1.10713393 | 2.27E−32 | 1.22E−29 | AGTR1 | angiotensin Il receptor type 1 |
| 0.839804157 | 7.23E−32 | 3.63E−29 | USP53 | ubiquitin specific peptidase 53 |
| 0.809826411 | 8.99E−13 | 3.54E−11 | SFRP2 | secreted frizzled related protein 2 |
| 0.774322761 | 1.93E−15 | 1.15E−13 | SKP2 | S-phase kinase associated protein 2 |
| 0.823885658 | 2.84E−09 | 5.63E−08 | SLC2A12 | solute carrier family 2 member 12 |
| 0.846823888 | 1.67E−26 | 4.27E−24 | TMEM47 | transmembrane protein 47 |
| 1.24530336 | 1.72E−45 | 2.97E−42 | GPC3 | glypican 3 |
| 0.819468058 | 5.45E−09 | 1.03E−07 | SHC3 | SHC adaptor protein 3 |
| 1.393667283 | 7.69E−11 | 2.07E−09 | ANKRD1 | ankyrin repeat domain 1 |
| 1.438840349 | 1.22E−08 | 2.12E−07 | TMC2 | transmembrane channel like 2 |
| 1.215806948 | 8.04E−47 | 1.56E−43 | TAGLN | transgelin |
| 1.260385153 | 1.52E−20 | 1.96E−18 | MKX | mohawk homeobox |
| 0.786465626 | 9.81E−18 | 8.58E−16 | CRIM1 | cysteine rich transmembrane BMP regulator 1 |
| 0.759909417 | 3.97E−15 | 2.25E−13 | SLC7A11 | solute carrier family 7 member 11 |
| 0.939257945 | 7.09E−13 | 2.84E−11 | ANO4 | anoctamin 4 |
| 1.008444048 | 6.84E−33 | 3.95E−30 | GFRA1 | GDNF family receptor alpha 1 |
| 0.829257541 | 2.48E−16 | 1.69E−14 | SETBP1 | SET binding protein 1 |
| 1.281366066 | 2.94E−25 | 6.45E−23 | IGSF10 | immunoglobulin superfamily member 10 |
| 0.890908924 | 3.49E−18 | 3.28E−16 | DDAH1 | dimethylarginine dimethylaminohydrolase 1 |
| 0.832921287 | 6.05E−10 | 1.38E−08 | SEMA3D | semaphorin 3D |
| 0.834372541 | 5.79E−11 | 1.60E−09 | AK5 | adenylate kinase 5 |
| 0.896163604 | 7.09E−11 | 1.92E−09 | PDE1C | phosphodiesterase 1C |
| 1.129226438 | 9.86E−09 | 1.76E−07 | JAM2 | junctional adhesion molecule 2 |
| 0.873740589 | 4.85E−40 | 5.81E−37 | ADAMTS5 | ADAM metallopeptidase with |
| | | | | thrombospondin type 1 motif 5 |
| 1.960537248 | 1.19E−19 | 1.37E−17 | GRIA1 | glutamate ionotropic receptor AMPA type |
| | | | | subunit 1 |
| 1.141120061 | 5.30E−33 | 3.30E−30 | DEPTOR | DEP domain containing MTOR interacting |
| | | | | protein |
| 1.517354686 | 2.06E−12 | 7.57E−11 | GDF6 | growth differentiation factor 6 |
| 1.034169103 | 9.98E−08 | 1.44E−06 | KCNS2 | potassium voltage-gated channel modifier |
| | | | | subfamily S member 2 |
| 0.85599469 | 3.40E−31 | 1.51E−28 | C1R | complement C1r |
| 0.894539454 | 1.03E−08 | 1.82E−07 | FAM131B | family with sequence similarity 131 |
| | | | | member B |
| 0.965721889 | 1.45E−56 | 1.13E−52 | PLPP3 | phospholipid phosphatase 3 |
| 1.53060429 | 5.33E−10 | 1.24E−08 | WNT4 | Wnt family member 4 |
| 0.797794249 | 4.77E−20 | 5.76E−18 | NEXN | nexilin F-actin binding protein |
| 0.877319305 | 2.49E−13 | 1.08E−11 | ADGRL4 | adhesion G protein-coupled receptor L4 |
| 2.156691955 | 3.04E−07 | 3.93E−06 | B3GALT2 | beta-1,3-galactosyltransferase 2 |
| 1.411284767 | 1.90E−10 | 4.74E−09 | NTNG1 | netrin G1 |
| 0.762101802 | 1.72E−08 | 2.91E−07 | OLFML2B | olfactomedin like 2B |
| 1.462371194 | 7.47E−10 | 1.67E−08 | FRZB | frizzled related protein |
| 1.505537868 | 3.44E−09 | 6.68E−08 | C1QTNF7 | C1q and TNF related 7 |
| 0.937005023 | 1.43E−42 | 2.02E−39 | FSTL1 | follistatin like 1 |
| 0.834430892 | 8.35E−15 | 4.50E−13 | RPL22L1 | ribosomal protein L22 like 1 |
| 0.80366448 | 7.65E−21 | 1.02E−18 | PTX3 | pentraxin 3 |
| 2.341228498 | 7.53E−09 | 1.38E−07 | DNASE1L3 | deoxyribonuclease 1 like 3 |
| 0.852104012 | 1.86E−11 | 5.77E−10 | HAND2 | heart and neural crest derivatives |
| | | | | expressed 2 |
| 1.417587954 | 9.23E−17 | 6.88E−15 | PI16 | peptidase inhibitor 16 |
| 1.093482639 | 1.71E−17 | 1.41E−15 | TNFRSF11B | TNF receptor superfamily member 11b |
| 1.001296849 | 4.97E−23 | 8.51E−21 | CTHRC1 | collagen triple helix repeat containing 1 |
| 1.620266405 | 1.06E−13 | 4.79E−12 | DIRAS2 | DIRAS family GTPase 2 |
| 1.009772629 | 1.19E−27 | 3.36E−25 | SVEP1 | sushi, von Willebrand factor type A, EGF |
| | | | | and pentraxin domain containing 1 |
| 1.816673686 | 3.20E−17 | 2.57E−15 | FAT3 | FAT atypical cadherin 3 |
| 0.799862269 | 4.42E−31 | 1.86E−28 | FBN1 | fibrillin 1 |
| 1.008400775 | 3.02E−29 | 1.03E−26 | WEE1 | WEE1 G2 checkpoint kinase |
| 1.539043298 | 3.24E−30 | 1.29E−27 | GREM1 | gremlin 1, DAN family BMP antagonist |
| 1.760103661 | 1.23E−08 | 2.14E−07 | FAM107A | family with sequence similarity 107 |
| | | | | member A |
| 1.125215966 | 6.05E−10 | 1.38E−08 | RAB3B | RAB3B, member RAS oncogene family |
| 1.210038265 | 2.11E−08 | 3.51E−07 | NLGN1 | neuroligin 1 |
| 0.855149212 | 1.59E−20 | 2.03E−18 | ALCAM | activated leukocyte cell adhesion molecule |
| 1.167706091 | 1.02E−07 | 1.46E−06 | SMAGP | small cell adhesion glycoprotein |
| 1.308300069 | 1.01E−68 | 1.58E−64 | PDGFD | platelet derived growth factor D |
| 0.922926098 | 6.88E−16 | 4.28E−14 | NEGR1 | neuronal growth regulator 1 |
| 0.849916479 | 8.88E−20 | 1.05E−17 | SYNPO2 | synaptopodin 2 |
| 1.301210617 | 3.04E−07 | 3.93E−06 | HPSE | heparanase |
| 0.964223021 | 4.44E−16 | 2.94E−14 | NDNF | neuron derived neurotrophic factor |
| 1.200087399 | 5.71E−14 | 2.72E−12 | DAB1 | DAB adaptor protein 1 |
| 0.839086576 | 1.96E−25 | 4.37E−23 | HEG1 | heart development protein with EGF like |
| | | | | domains 1 |
| 0.867820619 | 9.61E−29 | 3.18E−26 | MSRB3 | methionine sulfoxide reductase B3 |
| 0.758838942 | 6.35E−16 | 4.00E−14 | ADAMTSL1 | ADAMTS like 1 |
| 0.778601075 | 7.57E−36 | 6.55E−33 | MCFD2 | multiple coagulation factor deficiency 2, |
| | | | | Lipid cargo receptor complex subunit |
| 1.077661748 | 3.32E−25 | 7.18E−23 | TMEM64 | transmembrane protein 64 |
| 0.9142076 | 1.04E−17 | 9.05E−16 | TMEM45A | transmembrane protein 45A |
| 0.87930271 | 3.45E−32 | 1.79E−29 | PAPPA | pappalysin 1 |
| 0.801430144 | 2.32E−22 | 3.73E−20 | GAS6 | growth arrest specific 6 |
| 1.067260346 | 3.00E−20 | 3.77E−18 | CCBE1 | collagen and calcium binding EGF domains 1 |
| 0.772322682 | 8.82E−08 | 1.29E−06 | GPR1 | G protein-coupled receptor 1 |
| 1.552909203 | 5.55E−11 | 1.54E−09 | KCTD16 | potassium channel tetramerization domain |
| | | | | containing 16 |
| 0.809575707 | 4.65E−24 | 9.05E−22 | ALDH1A3 | aldehyde dehydrogenase 1 family |
| | | | | member A3 |
| 0.880350918 | 2.67E−12 | 9.56E−11 | KCND2 | potassium voltage-gated channel |
| | | | | subfamily D member 2 |
| 0.820014966 | 9.94E−17 | 7.30E−15 | PROS1 | protein S |
| 0.800120731 | 6.89E−19 | 7.02E−17 | ROR1 | receptor tyrosine kinase like orphan |
| | | | | receptor 1 |
| 1.314922398 | 3.96E−33 | 2.57E−30 | LSAMP | limbic system associated membrane protein |
| 0.945787726 | 4.73E−10 | 1.11E−08 | THSD4 | thrombospondin type 1 domain containing 4 |
| 0.943750183 | 2.90E−28 | 9.04E−26 | FAT4 | FAT atypical cadherin 4 |
| 0.820976928 | 9.21E−30 | 3.50E−27 | LAMA2 | laminin subunit alpha 2 |
| 0.846960903 | 1.73E−07 | 2.34E−06 | HRH1 | histamine receptor H1 |
| 0.773976924 | 6.83E−07 | 8.22E−06 | VEPH1 | ventricular zone expressed PH domain |
| | | | | containing 1 |
| 0.872190711 | 1.22E−19 | 1.37E−17 | MFAP5 | microfibril associated protein 5 |
| 1.178831668 | 1.14E−14 | 6.06E−13 | MAP1LC3C | microtubule associated protein 1 light |
| | | | | chain 3 gamma |
| 0.829226457 | 6.70E−33 | 3.95E−30 | LPAR1 | lysophosphatidic acid receptor 1 |
| 1.022764381 | 2.44E−27 | 6.55E−25 | ITGBL1 | integrin subunit beta like 1 |
| 0.75385608 | 3.74E−07 | 4.72E−06 | EGFL6 | EGF like domain multiple 6 |
| 0.881718791 | 1.58E−16 | 1.13E−14 | ALPK2 | alpha kinase 2 |
| 0.881615447 | 3.33E−19 | 3.50E−17 | GRK5 | G protein-coupled receptor kinase 5 |
| 0.851131452 | 5.39E−12 | 1.82E−10 | DMD | dystrophin |
| 0.896489557 | 4.81E−14 | 2.33E−12 | LAYN | layilin |
| 1.492029268 | 3.45E−11 | 1.00E−09 | AARD | alanine and arginine rich domain containing |
| | | | | protein |
| 0.787154869 | 1.10E−11 | 3.55E−10 | C4orf46 | chromosome 4 open reading frame 46 |
| 0.934538372 | 6.73E−21 | 9.04E−19 | VIT | vitrin |
| 1.043059359 | 2.36E−14 | 1.19E−12 | DOK6 | docking protein 6 |
| 0.897319675 | 6.47E−30 | 2.52E−27 | VGLL3 | vestigial like family member 3 |
| 0.848895197 | 1.14E−17 | 9.85E−16 | LBH | LBH regulator of WNT signaling pathway |
| 0.801278912 | 3.08E−09 | 6.05E−08 | ITGA1 | integrin subunit alpha 1 |
| 0.885142696 | 1.09E−16 | 7.94E−15 | PNMA2 | PNMA family member 2 |
| 1.102965935 | 8.05E−17 | 6.06E−15 | INMT | indolethylamine N-methyltransferase |
| 1.495749384 | 2.51E−52 | 1.30E−48 | LINC00968 | long intergenic non-protein coding RNA 968 |
| 1.360930375 | 1.33E−07 | 1.85E−06 | LINC01085 | long intergenic non-protein coding RNA 1085 |
| 1.069015559 | 1.79E−14 | 9.28E−13 | TRNP1 | TMF1 regulated nuclear protein 1 |
| 0.896637324 | 4.45E−40 | 5.77E−37 | MARCKS | myristoylated alanine rich protein kinase C |
| | | | | substrate |
| 1.190318913 | 3.53E−07 | 4.48E−06 | NEFL | neurofilament light |

TABLE 21
rs12454712-mediated LipocyteProfiler in visceral AMSCs at day14. (ANOVA adj.
BMI, sex, age, batch, significance level 5% FDR). P-value, p-value of ANOVA, q-value,
q-value of ANOVA, FDR; eta_sq, eta square of ANOVA, effect size; F value of ANOVA;
t-statistics of t-test.

| Lipocyte Profiler features | p-value | q-value | eta_sq | t-statistics |
|---|---|---|---|---|
| Cells_Intensity_MADIntensity_Mito | 1.02E−03 | 3.86E−02 | 0.47 | −4.18 |
| Cells_Intensity_MeanIntensity_Mito | 9.39E−04 | 3.86E−02 | 0.47 | −3.88 |
| Cells_Intensity_MedianIntensity_Mito | 5.60E−03 | 3.86E−02 | 0.37 | −3.43 |
| Cells_Intensity_UpperQuartileIntensity_Mito | 1.18E−03 | 3.86E−02 | 0.45 | −3.75 |
| Cytoplasm_Intensity_MADIntensity_Mito | 1.77E−03 | 3.86E−02 | 0.45 | −4.02 |
| Cytoplasm_Intensity_MeanIntensity_Mito | 1.46E−03 | 3.86E−02 | 0.45 | −3.66 |
| Cytoplasm_Intensity_MeanIntensityEdge_Mito | 6.86E−03 | 4.14E−02 | 0.33 | −2.88 |
| Cytoplasm_Intensity_MedianIntensity_Mito | 9.58E−03 | 4.94E−02 | 0.33 | −3.15 |
| Cytoplasm_Intensity_StdIntensityEdge_Mito | 9.78E−03 | 4.98E−02 | 0.29 | −3.10 |
| Cytoplasm_Intensity_UpperQuartileIntensity_Mito | 1.73E−03 | 3.86E−02 | 0.44 | −3.62 |
| Nuclei_Granularity_4_BODIPY | 6.98E−03 | 4.14E−02 | 0.16 | 2.57 |
| Nuclei_Intensity_MinIntensity_Mito | 5.85E−03 | 3.92E−02 | 0.33 | −3.53 |
| Nuclei_Intensity_MinIntensityEdge_Mito | 7.06E−03 | 4.14E−02 | 0.32 | −3.43 |
| Cells_AreaShape_Zernike_2_2 | 9.50E−03 | 4.93E−02 | 0.26 | −2.27 |
| Cells_Neighbors_NumberOfNeighbors_10 | 5.50E−03 | 3.86E−02 | 0.32 | 2.96 |
| Cells_Texture_Contrast_Mito_10_01 | 4.65E−03 | 3.86E−02 | 0.37 | −2.87 |
| Cells_Texture_DifferenceEntropy_Mito_5_02 | 6.16E−03 | 3.99E−02 | 0.35 | −3.73 |
| Cells_Texture_Entropy_Mito_5_02 | 3.30E−03 | 3.86E−02 | 0.39 | −4.31 |
| Cells_Texture_InverseDifferenceMoment_Mito_10_02 | 4.15E−03 | 3.86E−02 | 0.37 | 4.17 |
| Cells_Texture_SumAverage_Mito_10_02 | 9.72E−04 | 3.86E−02 | 0.46 | −3.94 |
| Cells_Texture_SumEntropy_Mito_5_00 | 2.97E−03 | 3.86E−02 | 0.38 | −4.53 |
| Nuclei_Texture_Entropy_DNA_20_01 | 3.56E−03 | 3.86E−02 | 0.27 | 2.36 |
| Nuclei_Texture_InfoMeas1_Mito_10_00 | 1.93E−03 | 3.86E−02 | 0.40 | 5.95 |
| Cytoplasm_Texture_Contrast_Mito_10_02 | 4.64E−03 | 3.86E−02 | 0.37 | −2.77 |
| Cytoplasm_Texture_DifferenceEntropy_Mito_5_02 | 5.97E−03 | 3.97E−02 | 0.36 | −3.72 |
| Cytoplasm_Texture_Entropy_Mito_5_02 | 3.54E−03 | 3.86E−02 | 0.40 | −4.25 |
| Cytoplasm_Texture_InverseDifferenceMoment_Mito_20_02 | 3.85E−03 | 3.86E−02 | 0.38 | 4.30 |
| Cytoplasm_Texture_SumAverage_Mito_5_02 | 1.49E−03 | 3.86E−02 | 0.45 | −3.69 |
| Cytoplasm_Texture_SumEntropy_Mito_10_00 | 3.21E−03 | 3.86E−02 | 0.39 | −4.47 |

TABLE 22
Lists of pathways enriched among significant connections between gene and LP features of
WHRadjBMI profile. Term, which pathway; Overlap, number of genes that overlap
and total genes; P-value, enrichment p-value; Adjusted P-value, Q-value; Odds Ratio,
enrichment; Genes, genes in the pathway which are associated with gene LP-feature connections.

| | | | adj. | | |
|---|---|---|---|---|---|
| Term | Overlap | p-value | p-value | OR | Genes |
| Generic transcription pathway | 15/377 | 2.97E−04 | 1.77E−01 | 3.03 | ZNF551; ZNF561; ZNF263; THRA; RBPJ; MED4; RXRA; |
| | | | | | TFDP1; ZNF517; ZKSCAN3; ZNF416; ZNF138; ZNF567; |
| | | | | | ZNF225; ZNF112 |
| Apoptosis intrinsic pathway | 4/30 | 7.73E−04 | 2.31E−01 | 10.98 | BCL2L11; TFDP1; DYNLL2; BCL2L1 |
| Activation of BH3-only proteins | 3/17 | 1.60E−03 | 3.09E−01 | 15.24 | BCL2L11; TFDP1; DYNLL2 |
| G1 to S cell cycle control | 5/67 | 2.47E−03 | 3.09E−01 | 5.76 | CDKN1C; CCNB1; CDKN1B; TFDP1; CCNG2 |
| Valine, leucine and isoleucine | 4/44 | 3.29E−03 | 3.09E−01 | 7.13 | ALDH6A1; ALDH2; EHHADH; DBT |
| degradation | | | | | |
| Cyclins and cell cycle regulation | 3/23 | 3.91E−03 | 3.09E−01 | 10.67 | CCNB1; CDKN1B; TFDP1 |
| Inactivation of BCL-2 by BH3- | 2/7 | 3.92E−03 | 3.09E−01 | 28.37 | BCL2L11; BCL2L1 |
| only proteins | | | | | |
| FoxO family signaling | 4/49 | 4.86E−03 | 3.09E−01 | 6.34 | CCNB1; CDKN1B; BCL2L11; BCL6 |
| APC/C-and Cdc20-mediated | 3/25 | 4.97E−03 | 3.09E−01 | 9.70 | CCNB1; NEK2; ANAPC10 |
| degradation of Nek2A | | | | | |
| Mitotic metaphase/anaphase | 2/8 | 5.17E−03 | 3.09E−01 | 23.64 | STAG1; REC8 |
| transition | | | | | |
| Notch signaling pathway | 6/121 | 7.18E−03 | 3.90E−01 | 3.73 | EFNB2; MFAP2; DTX1; MAPT; RBPJ; BCL2L1 |
| Deregulation of CDK5 in | 2/10 | 8.16E−03 | 4.06E−01 | 17.73 | CAPNS1; MAPT |
| Alzheimer's disease | | | | | |
| SUMOylation as a mechanism to | 2/11 | 9.88E−03 | 4.54E−01 | 15.76 | SUMO1; UBA1 |
| modulate CtBP-dependent gene | | | | | |
| responses | | | | | |
| Propanoate metabolism | 3/33 | 1.09E−02 | 4.63E−01 | 7.11 | ALDH6A1; ALDH2; EHHADH |
| PIK3C1/AKT pathway | 3/35 | 1.28E−02 | 5.08E−01 | 6.66 | CDKN1B; TBC1D4; BCL2L1 |
| p27 phosphorylation regulation | 2/13 | 1.38E−02 | 5.13E−01 | 12.89 | CDKN1B; TFDP1 |
| during cell cycle progression | | | | | |
| MAP kinase inactivation of | 2/14 | 1.59E−02 | 5.17E−01 | 11.82 | RXRA; THRA |
| SMRT corepressor | | | | | |
| Triglyceride biosynthesis | 3/38 | 1.60E−02 | 5.17E−01 | 6.09 | ELOVL5; GPAT2; LPIN1 |
| AP-1 transcription factor | 4/70 | 1.68E−02 | 5.17E−01 | 4.32 | CDKN1B; BCL2L11; NFATC3; CCL2 |
| network | | | | | |
| AKAP95 role in mitosis and | 2/15 | 1.82E−02 | 5.17E−01 | 10.91 | CCNB1; NCAPD2 |
| chromosome dynamics | | | | | |
| Fibrinolysis pathway | 2/15 | 1.82E−02 | 5.17E−01 | 10.91 | F2R; UBA1 |
| FOXM1 transcription factor | 3/41 | 1.96E−02 | 5.31E−01 | 5.61 | CCNB1; NFATC3; NEK2 |
| network | | | | | |
| Fatty acid metabolism | 3/42 | 2.09E−02 | 5.41E−01 | 5.47 | GCDH; ALDH2; EHHADH |
| Branched-chain amino acid | 2/17 | 2.31E−02 | 5.55E−01 | 9.45 | ALDH6A1; DBT |
| catabolism | | | | | |
| Lysine degradation | 3/44 | 2.36E−02 | 5.55E−01 | 5.20 | GCDH; ALDH2; EHHADH |
| Immune system | 22/998 | 2.42E−02 | 5.55E−01 | 1.64 | CDKN1B; SH3KBP1; NCF2; MX2; TNFAIP3; AP2B1; UBE2Z; |
| | | | | | DYNLL2; ANAPC10; DDOST; PJA1; PSMA3; SUMO1; CASP4; |
| | | | | | TLR9; HLA-DRA; UBA1; VHL; TRIM21; CD44; HLA-DPA1; |
| | | | | | BCL2L1 |
| Antigen presentation: folding, | 8/255 | 2.77E−02 | 5.59E−01 | 2.32 | PSMA3; NCF2; UBA1; VHL; UBE2Z; TRIM21; ANAPC10; PJA1 |
| assembly, and peptide loading | | | | | |
| of class I MHC proteins | | | | | |
| Nucleotide-binding domain, | 3/47 | 2.80E−02 | 5.59E−01 | 4.84 | CASP4; TNFAIP3; BCL2L1 |
| leucine rich repeat containing | | | | | |
| receptor (NLR) signaling | | | | | |
| pathways | | | | | |
| APC/C-mediated degradation of | 4/82 | 2.81E−02 | 5.59E−01 | 3.65 | PSMA3; CCNB1; PTTG1; ANAPC10 |
| cell cycle proteins | | | | | |
| ATM-dependent DNA damage | 4/82 | 2.81E−02 | 5.59E−01 | 3.65 | CDKN1B; BCL2L11; BCL6; CCNG2 |
| response | | | | | |
| Glycerolipid metabolism | 3/49 | 3.12E−02 | 5.79E−01 | 4.63 | ALDH2; PNPLA3; GPAT2 |
| NOD signaling pathway | 4/85 | 3.15E−02 | 5.79E−01 | 3.51 | CASP4; CARD6; TNFAIP3; CCL2 |
| Glycerophospholipid | 4/86 | 3.27E−02 | 5.79E−01 | 3.47 | PNPLA3; GPAT2; LPIN1; CDS2 |
| biosynthesis | | | | | |
| APC/C activator regulation | 2/21 | 3.44E−02 | 5.79E−01 | 7.46 | CCNB1; ANAPC10 |
| between G1/S and early | | | | | |
| anaphase | | | | | |
| Biosynthesis of unsaturated | 2/21 | 3.44E−02 | 5.79E−01 | 7.46 | ELOVL5; SCD5 |
| fatty acids | | | | | |
| Interleukin-2 receptor beta | 3/52 | 3.63E−02 | 5.79E−01 | 4.35 | CCNB1; TFDP1; BCL2L1 |
| chain in T cell activation | | | | | |
| Beta-alanine metabolism | 2/22 | 3.75E−02 | 5.79E−01 | 7.09 | ALDH2; EHHADH |
| Thrombin signaling through | 3/53 | 3.81E−02 | 5.79E−01 | 4.26 | ARFGEF1; F2R; GNAI1 |
| protease-activated receptors | | | | | |
| Ubiquitin-mediated proteolysis | 5/136 | 4.26E−02 | 5.79E−01 | 2.72 | HLipidC3; UBA1; VHL; UBE2Z; ANAPC10 |
| Proteasome complex | 2/24 | 4.40E−02 | 5.79E−01 | 6.44 | PSMA3; UBA1 |
| p53 signaling pathway | 5/139 | 4.61E−02 | 5.79E−01 | 2.66 | CCNB1; TFDP1; BCL6; TRRAP; BCL2L1 |
| Adaptive immune system | 14/606 | 4.69E−02 | 5.79E−01 | 1.70 | CDKN1B; SH3KBP1; NCF2; AP2B1; UBE2Z; DYNLL2; |
| | | | | | ANAPC10; PJA1; PSMA3; HLA-DRA; UBA1; VHL; |
| | | | | | TRIM21; HLA-DPA1 |
| Interferon-gamma signaling | 4/97 | 4.74E−02 | 5.79E−01 | 3.06 | SUMO1; HLA-DRA; CD44; HLA-DPA1 |
| pathway | | | | | |
| Tryptophan metabolism | 3/58 | 4.77E−02 | 5.79E−01 | 3.87 | GCDH; ALDH2; EHHADH |
| Senescence and autophagy | 4/99 | 5.05E−02 | 5.79E−01 | 2.99 | CDKN1B; LAMP1; KMT2A; CD44 |
| RXR/VDR pathway | 2/26 | 5.09E−02 | 5.79E−01 | 5.90 | RXRA; THRA |
| Gene expression | 20/968 | 5.38E−02 | 5.79E−01 | 1.52 | ZNF551; ZNF561; ZNF263; EIF2B2; THRA; SSRP1; RBPJ; |
| | | | | | MED4; DDOST; PSMA3; RXRA; TFDP1; ZNF517; ZKSCAN3; |
| | | | | | ZNF416; ZNF138; ZNF567; ZNF225; SF3B1; ZNF112 |
| TWEAK regulation of gene | 2/27 | 5.45E−02 | 5.79E−01 | 5.67 | CCL2; BCL2L1 |
| expression | | | | | |
| EGFR downregulation | 2/27 | 5.45E−02 | 5.79E−01 | 5.67 | SH3KBP1; AP2B1 |
| Apoptosis | 7/242 | 5.46E−02 | 5.79E−01 | 2.13 | PSMA3; BCL2L11; TFDP1; CASP4; MAPT; DYNLL2; BCL2L1 |
| Cell cycle | 11/453 | 5.50E−02 | 5.79E−01 | 1.78 | CDKN1C; STAG1; PSMA3; CCNB1; CDKN1B; TFDP1; PTTG1; |
| | | | | | NEK2; ANAPC10; REC8; GADD45G |
| TGF-beta regulation of | 13/565 | 5.56E−02 | 5.79E−01 | 1.69 | CDKN1C; B3GALNT1; CYB5A; JUP; BTN2A1; F2R; TNFAIP3; |
| extracellular matrix | | | | | ZNF35; SAT1; TFDP1; PTTG1; NCK2; BCL2L1 |
| MHC class II antigen | 4/103 | 5.68E−02 | 5.79E−01 | 2.87 | HLA-DRA; AP2B1; DYNLL2; HLA-DPA1 |
| presentation | | | | | |
| Cell cycle: G1/S checkpoint | 2/28 | 5.82E−02 | 5.79E−01 | 5.45 | CDKN1B; TFDP1 |
| ADP-ribosylation factor | 2/29 | 6.19E−02 | 5.79E−01 | 5.25 | ARFGEF1; NCF2 |
| Reelin signaling pathway | 2/29 | 6.19E−02 | 5.79E−01 | 5.25 | NCK2; MAPT |
| Influence of Ras and Rho | 2/29 | 6.19E−02 | 5.79E−01 | 5.25 | CDKN1B; TFDP1 |
| proteins on G1 to S transition | | | | | |
| Asthma | 2/30 | 6.57E−02 | 5.79E−01 | 5.06 | HLA-DRA; HLA-DPA1 |
| Butanoate metabolism | 2/30 | 6.57E−02 | 5.79E−01 | 5.06 | EHHADH; ACSM5 |
| Activation of NOXA and | 1/5 | 6.81E−02 | 5.79E−01 | 17.67 | TFDP1 |
| translocation to mitochondria | | | | | |
| Acyl chain remodeling of | 1/5 | 6.81E−02 | 5.79E−01 | 17.67 | PNPLA3 |
| diacylglycerol and triacylglycerol | | | | | |
| Chk1/Chk2(Cds1)-mediated | 1/5 | 6.81E−02 | 5.79E−01 | 17.67 | CCNB1 |
| inactivation of cyclin B-Cdk1 | | | | | |
| complex | | | | | |
| CD40/CD40L signaling | 2/31 | 6.96E−02 | 5.79E−01 | 4.88 | TNFAIP3; BCL2L1 |
| PD-1 signaling | 2/31 | 6.96E−02 | 5.79E−01 | 4.88 | HLA-DRA; HLA-DPA1 |
| PPAR signaling pathway | 3/69 | 7.25E−02 | 5.79E−01 | 3.23 | RXRA; EHHADH; SCD5 |
| Oocyte meiosis | 4/113 | 7.44E−02 | 5.79E−01 | 2.61 | CCNB1; PTTG1; ANAPC10; REC8 |
| MicroRNA regulation of DNA | 3/70 | 7.50E−02 | 5.79E−01 | 3.18 | CCNB1; CDKN1B; GADD45G |
| damage response | | | | | |
| E2F-mediated regulation of DNA | 2/33 | 7.77E−02 | 5.79E−01 | 4.57 | CCNB1; TFDP1 |
| replication | | | | | |
| Leishmaniasis | 3/72 | 8.01E−02 | 5.79E−01 | 3.08 | NCF2; HLA-DRA; HLA-DPA1 |
| Phosphorylation of Emi1 | 1/6 | 8.11E−02 | 5.79E−01 | 14.13 | CCNB1 |
| Proteolysis and signaling | 1/6 | 8.11E−02 | 5.79E−01 | 14.13 | RBPJ |
| pathway of Notch | | | | | |
| Pyrimidine biosynthesis | 1/6 | 8.11E−02 | 5.79E−01 | 14.13 | DHODH |
| Basic mechanisms of | 1/6 | 8.11E−02 | 5.79E−01 | 14.13 | SUMO1 |
| SUMOylation | | | | | |
| Low-density lipoprotein (LDL) | 1/6 | 8.11E−02 | 5.79E−01 | 14.13 | CCL2 |
| pathway during atherogenesis | | | | | |
| MSP/RON receptor signaling | 1/6 | 8.11E−02 | 5.79E−01 | 14.13 | CCL2 |
| pathway | | | | | |
| Neurotransmitter clearance in | 1/6 | 8.11E−02 | 5.79E−01 | 14.13 | ALDH2 |
| the synaptic cleft | | | | | |
| Cell cycle checkpoints | 4/117 | 8.22E−02 | 5.79E−01 | 2.51 | CCNB1; PSMA3; CDKN1B; ANAPC10 |
| RANKL regulation of apoptosis | 3/74 | 8.54E−02 | 5.79E−01 | 3.00 | DTX1; CCL2; CD44 |
| and immune response | | | | | |
| E2F transcription factor network | 3/74 | 8.54E−02 | 5.79E−01 | 3.00 | CDKN1B; TFDP1; TRRAP |
| EGF receptor (ErbB1) signaling | 2/35 | 8.60E−02 | 5.79E−01 | 4.29 | NCK2; GNAI1 |
| pathway | | | | | |
| fMLP induced chemokine gene | 2/35 | 8.60E−02 | 5.79E−01 | 4.29 | NCF2; NFATC3 |
| expression in HMC-1 cells | | | | | |
| Interleukin-7 signaling pathway | 2/35 | 8.60E−02 | 5.79E−01 | 4.29 | CDKN1B; BCL2L1 |
| MEF2D role in T cell apoptosis | 2/35 | 8.60E−02 | 5.79E−01 | 4.29 | CAPNS1; HLA-DRA |
| Interferon signaling | 5/168 | 8.77E−02 | 5.79E−01 | 2.18 | SUMO1; MX2; HLA-DRA; CD44; HLA-DPA1 |
| Generation of second | 2/36 | 9.02E−02 | 5.79E−01 | 4.17 | HLA-DRA; HLA-DPA1 |
| messenger molecules | | | | | |
| Phosphatidylglycerol | 1/7 | 9.40E−02 | 5.79E−01 | 11.78 | CDS2 |
| biosynthesis | | | | | |
| FXR and LXR regulation of | 1/7 | 9.40E−02 | 5.79E−01 | 11.78 | RXRA |
| cholesterol metabolism | | | | | |
| Gene expression regulation in | 1/7 | 9.40E−02 | 5.79E−01 | 11.78 | RBPJ |
| late stage pancreatic bud | | | | | |
| precursor cells | | | | | |
| Nef-mediated CD8 | 1/7 | 9.40E−02 | 5.79E−01 | 11.78 | AP2B1 |
| downregulation | | | | | |
| Allograft rejection | 2/37 | 9.45E−02 | 5.79E−01 | 4.05 | HLA-DRA; HLA-DPA1 |
| FRA pathway | 2/37 | 9.45E−02 | 5.79E−01 | 4.05 | NFATC3; CCL2 |
| Peroxisome | 3/78 | 9.63E−02 | 5.79E−01 | 2.84 | EHHADH; AGPS; PXMP2 |
| Fatty acid, triacylglycerol, and | 5/173 | 9.64E−02 | 5.79E−01 | 2.12 | RXRA; ELOVL5; GPAT2; MED4; LPIN1 |
| ketone body metabolism | | | | | |
| Signaling events mediated by | 2/38 | 9.89E−02 | 5.79E−01 | 3.93 | SUMO1; BCL6 |
| HDAC class II | | | | | |
| Cyclin D-associated events in G1 | 2/38 | 9.89E−02 | 5.79E−01 | 3.93 | CDKN1B; TFDP1 |
| Nuclear receptors | 2/38 | 9.89E−02 | 5.79E−01 | 3.93 | RXRA; THRA |
| Apoptosis modulation and | 3/80 | 1.02E−01 | 5.79E−01 | 2.76 | BCL2L11; CAPNS1; BCL2L1 |
| signaling | | | | | |
| Aurora B signaling | 2/39 | 1.03E−01 | 5.79E−01 | 3.83 | PSMA3; NCAPD2 |
| Recycling of elF2-GDP complex | 1/8 | 1.07E−01 | 5.79E−01 | 10.09 | EIF2B2 |
| BRCA1-dependent ubiquitin | 1/8 | 1.07E−01 | 5.79E−01 | 10.09 | BARD1 |
| ligase activity | | | | | |
| Binding of RNA by insulin-like | 1/8 | 1.07E−01 | 5.79E−01 | 10.09 | CD44 |
| growth factor 2 mRNA binding | | | | | |
| proteins (IGF2BPs/IMPs/VICKZs) | | | | | |
| Bystander B cell activation | 1/8 | 1.07E−01 | 5.79E−01 | 10.09 | HLA-DRA |
| Chromatin remodeling by | 1/8 | 1.07E−01 | 5.79E−01 | 10.09 | RXRA |
| nuclear receptors to facilitate | | | | | |
| initiation of transcription in | | | | | |
| carcinoma cells | | | | | |
| Cross-presentation of | 1/8 | 1.07E−01 | 5.79E−01 | 10.09 | NCF2 |
| particulate exogenous antigens | | | | | |
| (phagosomes) | | | | | |
| Transport of nucleotide sugars | 1/8 | 1.07E−01 | 5.79E−01 | 10.09 | SLC35A1 |
| Visceral fat deposits and the | 1/8 | 1.07E−01 | 5.79E−01 | 10.09 | RXRA |
| metabolic syndrome | | | | | |
| Eosinophils in the chemokine | 1/8 | 1.07E−01 | 5.79E−01 | 10.09 | HLA-DRA |
| network of allergy | | | | | |
| Vitamin C (ascorbate) | 1/8 | 1.07E−01 | 5.79E−01 | 10.09 | CYB5A |
| metabolism | | | | | |
| Vitamin D biosynthesis | 1/8 | 1.07E−01 | 5.79E−01 | 10.09 | RXRA |
| Lysine catabolism | 1/8 | 1.07E−01 | 5.79E−01 | 10.09 | GCDH |
| Bioactive peptide-induced | 2/41 | 1.12E−01 | 5.85E−01 | 3.63 | MAPT; GNAI1 |
| signaling pathway | | | | | |
| Graft-versus-host disease | 2/41 | 1.12E−01 | 5.85E−01 | 3.63 | HLA-DRA; HLA-DPA1 |
| Small cell lung cancer | 3/84 | 1.14E−01 | 5.85E−01 | 2.63 | CDKN1B; RXRA; BCL2L1 |
| VEGF, hypoxia, and angiogenesis | 2/42 | 1.17E−01 | 5.85E−01 | 3.54 | EIF2B2; VHL |
| Alpha-4 beta-7 integrin signaling | 1/9 | 1.19E−01 | 5.85E−01 | 8.83 | CD44 |
| Bicarbonate transporters | 1/9 | 1.19E−01 | 5.85E−01 | 8.83 | SLC4A8 |
| G2/M DNA damage checkpoint | 1/9 | 1.19E−01 | 5.85E−01 | 8.83 | CCNB1 |
| Nef-mediated CD4 | 1/9 | 1.19E−01 | 5.85E−01 | 8.83 | AP2B1 |
| downregulation | | | | | |
| PPAR-gamma coactivator role in | 1/9 | 1.19E−01 | 5.85E−01 | 8.83 | RXRA |
| obesity and thermogenesis | | | | | |
| Progesterone-mediated oocyte | 3/86 | 1.20E−01 | 5.85E−01 | 2.56 | CCNB1; ANAPC10; GNAI1 |
| Type 1 diabetes mellitus | 2/43 | 1.21E−01 | 5.85E−01 | 3.45 | HLA-DRA; HLA-DPA1 |
| Mitotic prometaphase | 2/43 | 1.21E−01 | 5.85E−01 | 3.45 | STAG1; REC8 |
| TNF-alpha effects on cytokine | 4/135 | 1.22E−01 | 5.85E−01 | 2.17 | DBT; TNFAIP3; CCL2; CD44 |
| activity, cell motility, and | | | | | |
| apoptosis | | | | | |
| Mitotic G1-G1/S phases | 4/135 | 1.22E−01 | 5.85E−01 | 2.17 | CCNB1; PSMA3; CDKN1B; TFDP1 |
| RhoA signaling pathway | 2/45 | 1.31E−01 | 5.93E−01 | 3.29 | CDKN1B; CIT |
| T cell receptor signaling | 4/139 | 1.31E−01 | 5.93E−01 | 2.10 | NCF2; NFATC3; NCK2; HLA-DRA |
| pathway | | | | | |
| Activation of Src by protein | 1/10 | 1.32E−01 | 5.93E−01 | 7.85 | CCNB1 |
| tyrosine phosphatase alpha | | | | | |
| TRAF6-mediated IRF7 activation | 1/10 | 1.32E−01 | 5.93E−01 | 7.85 | TLR9 |
| in TLR7/8 or 9 signaling | | | | | |
| E2F-enabled inhibition of pre- | 1/10 | 1.32E−01 | 5.93E−01 | 7.85 | CCNB1 |
| replication complex formation | | | | | |
| Ethanol oxidation | 1/10 | 1.32E−01 | 5.93E−01 | 7.85 | ALDH2 |
| Metabolism | 28/1615 | 1.41E−01 | 5.93E−01 | 1.27 | GCDH; AHCYL1; COX17; ACSM5; ENO3; SAT1; GNAI1; |
| | | | | | ALDH2; DBT; FFAR4; GPAT2; MGAT2; B3GALNT1; FDPS; |
| | | | | | CYB5A; ELOVL5; GLCE; CMBL; MED4; DDOST; DHODH; |
| | | | | | ALDH6A1; EHHADH; AGPS; PNPLA3; LPIN1; CD44; CDS2 |
| ALK2 pathway | 1/11 | 1.44E−01 | 5.93E−01 | 7.06 | ACVR1 |
| Alpha-linolenic (omega3) and | 1/11 | 1.44E−01 | 5.93E−01 | 7.06 | ELOVL5 |
| linoleic (omega6) acid | | | | | |
| metabolism | | | | | |
| Recruitment of NuMA to mitotic | 1/11 | 1.44E−01 | 5.93E−01 | 7.06 | CCNB1 |
| centrosomes | | | | | |
| B lymphocyte cell surface | 1/11 | 1.44E−01 | 5.93E−01 | 7.06 | HLA-DRA |
| molecules | | | | | |
| Sonic Hedgehog (SHH) receptor | 1/11 | 1.44E−01 | 5.93E−01 | 7.06 | CCNB1 |
| PTCH1 regulation of cell cycle | | | | | |
| Down syndrome cell adhesion | 1/11 | 1.44E−01 | 5.93E−01 | 7.06 | DSCAML1 |
| molecule (DSCAM) interactions | | | | | |
| E2F1 destruction pathway | 1/11 | 1.44E−01 | 5.93E−01 | 7.06 | TFDP1 |
| p38 gamma/delta MAPK | 1/11 | 1.44E−01 | 5.93E−01 | 7.06 | MAPT |
| pathway | | | | | |
| Monocyte and its surface | 1/11 | 1.44E−01 | 5.93E−01 | 7.06 | CD44 |
| molecules | | | | | |
| LipidBB signaling pathway | 3/94 | 1.45E−01 | 5.93E−01 | 2.34 | CDKN1B; BCL2L11; NCK2 |
| Presenilin action in Notch and | 2/48 | 1.45E−01 | 5.93E−01 | 3.08 | DTX1; RBPJ |
| Wnt signaling | | | | | |
| HES/HEY pathway | 2/48 | 1.45E−01 | 5.93E−01 | 3.08 | CDKN1B; RBPJ |
| Intestinal immune network for | 2/48 | 1.45E−01 | 5.93E−01 | 3.08 | HLA-DRA; HLA-DPA1 |
| IgA production | | | | | |
| N-glycan biosynthesis | 2/48 | 1.45E−01 | 5.93E−01 | 3.08 | MGAT2; DDOST |
| Oncostatin M | 7/311 | 1.48E−01 | 5.93E−01 | 1.64 | CCNB1; CDKN1B; PTTG1; F2R; CASP4; CCL2; OSMR |
| Interleukin-5 signaling pathway | 2/49 | 1.50E−01 | 5.93E−01 | 3.01 | HLA-DRA; MAPT |
| Lipid and lipoprotein | 10/489 | 1.50E−01 | 5.93E−01 | 1.49 | FDPS; RXRA; ELOVL5; VAPB; AGPS; PNPLA3; GPAT2; MED4; |
| metabolism | | | | | LPIN1; CDS2 |
| Parkin role in the ubiquitin- | 1/12 | 1.56E−01 | 5.93E−01 | 6.42 | SUMO1 |
| proteasomal pathway | | | | | |
| Phosphatidylethanolamine | 1/12 | 1.56E−01 | 5.93E−01 | 6.42 | LPIN1 |
| biosynthesis | | | | | |
| Caspase-mediated cleavage of | 1/12 | 1.56E−01 | 5.93E−01 | 6.42 | MAPT |
| cytoskeletal proteins | | | | | |
| Toll-like receptor endosomal | 1/12 | 1.56E−01 | 5.93E−01 | 6.42 | TLR9 |
| trafficking and processing | | | | | |
| Inhibition of replication | 1/12 | 1.56E−01 | 5.93E−01 | 6.42 | TFDP1 |
| initiation of damaged DNA by | | | | | |
| RB1/E2F1 | | | | | |
| Malaria | 2/51 | 1.60E−01 | 5.93E−01 | 2.89 | TLR9; CCL2 |
| Nuclear receptor transcription | 2/51 | 1.60E−01 | 5.93E−01 | 2.89 | RXRA; THRA |
| pathway | | | | | |
| Phospholipid metabolism | 5/205 | 1.61E−01 | 5.93E−01 | 1.77 | VAPB; PNPLA3; GPAT2; LPIN1; CDS2 |
| Pyruvate metabolism | 2/52 | 1.65E−01 | 5.93E−01 | 2.83 | ALDH2; ACYP2 |
| Autoimmune thyroid disease | 2/52 | 1.65E−01 | 5.93E−01 | 2.83 | HLA-DRA; HLA-DPA1 |
| Mitochondrial protein import | 2/52 | 1.65E−01 | 5.93E−01 | 2.83 | COX17; TIMM22 |
| Adenylate cyclase inhibitory | 1/13 | 1.68E−01 | 5.93E−01 | 5.89 | GNAI1 |
| pathway | | | | | |
| Advanced glycosylation | 1/13 | 1.68E−01 | 5.93E−01 | 5.89 | DDOST |
| endproduct receptor signaling | | | | | |
| CBL-mediated ligand-induced | 1/13 | 1.68E−01 | 5.93E−01 | 5.89 | SH3KBP1 |
| downregulation of EGF | | | | | |
| receptors | | | | | |
| Lck and Fyn tyrosine kinases in | 1/13 | 1.68E−01 | 5.93E−01 | 5.89 | HLA-DRA |
| initiation of T cell receptor | | | | | |
| activation | | | | | |
| NICD trafficking to the nucleus | 1/13 | 1.68E−01 | 5.93E−01 | 5.89 | RBPJ |
| PI3K class IB pathway | 1/13 | 1.68E−01 | 5.93E−01 | 5.89 | GNAI1 |
| Androgen receptor regulation of | 2/53 | 1.70E−01 | 5.93E−01 | 2.77 | RXRA; SENP1 |
| biosynthesis and transcription | | | | | |
| Phagosome | 4/154 | 1.70E−01 | 5.93E−01 | 1.89 | LAMP1; NCF2; HLA-DRA; HLA-DPA1 |
| Arginine and proline | 2/54 | 1.75E−01 | 5.93E−01 | 2.72 | ALDH2; SAT1 |
| metabolism | | | | | |
| Antigen-activated B-cell | 5/211 | 1.75E−01 | 5.93E−01 | 1.72 | BCL6; SH3KBP1; NFATC3; TLR9; HLA-DRA |
| receptor generation of second | | | | | |
| messengers | | | | | |
| Chagas disease | 3/104 | 1.79E−01 | 5.93E−01 | 2.10 | TLR9; CCL2; GNAI1 |
| Interleukin-4 signaling pathway | 3/104 | 1.79E−01 | 5.93E−01 | 2.10 | BCL6; HLA-DRA; BCL2L1 |
| AKT phosphorylation of | 1/14 | 1.79E−01 | 5.93E−01 | 5.43 | CDKN1B |
| cytosolic targets | | | | | |
| Activation of Rac | 1/14 | 1.79E−01 | 5.93E−01 | 5.43 | NCK2 |
| Retrograde neurotrophin | 1/14 | 1.79E−01 | 5.93E−01 | 5.43 | AP2B1 |
| signaling | | | | | |
| S1P/S1P4 pathway | 1/14 | 1.79E−01 | 5.93E−01 | 5.43 | GNAI1 |
| CARM1 transcriptional | 1/14 | 1.79E−01 | 5.93E−01 | 5.43 | RXRA |
| regulation by protein | | | | | |
| methylation | | | | | |
| Glycosphingolipid biosynthesis: | 1/14 | 1.79E−01 | 5.93E−01 | 5.43 | B3GALNT1 |
| globo series | | | | | |
| Hyaluronan metabolism | 1/14 | 1.79E−01 | 5.93E−01 | 5.43 | CD44 |
| Interferon gamma signaling | 1/14 | 1.79E−01 | 5.93E−01 | 5.43 | SUMO1 |
| regulation | | | | | |
| Calcineurin-dependent NFAT | 2/55 | 1.80E−01 | 5.93E−01 | 2.67 | NFATC3; BCL2L1 |
| signaling role in lymphocytes | | | | | |
| Meiotic synapsis | 2/55 | 1.80E−01 | 5.93E−01 | 2.67 | STAG1; REC8 |
| Pre-NOTCH expression and | 2/57 | 1.90E−01 | 6.02E−01 | 2.57 | TFDP1; RBPJ |
| processing | | | | | |
| Mechanism of gene regulation | 2/57 | 1.90E−01 | 6.02E−01 | 2.57 | RXRA; EHHADH |
| by peroxisome proliferators via | | | | | |
| PPAR-alpha | | | | | |
| BMP signaling pathway in stem | 1/15 | 1.91E−01 | 6.02E−01 | 5.05 | ACVR1 |
| cells | | | | | |
| Cyclin A/B1-associated events | 1/15 | 1.91E−01 | 6.02E−01 | 5.05 | CCNB1 |
| during G2/M transition | | | | | |
| Terpenoid backbone | 1/15 | 1.91E−01 | 6.02E−01 | 5.05 | FDPS |
| biosynthesis | | | | | |
| Extrinsic prothrombin activation | 1/15 | 1.91E−01 | 6.02E−01 | 5.05 | F2R |
| pathway | | | | | |
| Fatty acid omega oxidation | 1/15 | 1.91E−01 | 6.02E−01 | 5.05 | ALDH2 |
| Mu-calpain pathway | 1/15 | 1.91E−01 | 6.02E−01 | 5.05 | CAPNS1 |
| Autodegradation of Cdh1 by | 2/58 | 1.95E−01 | 6.04E−01 | 2.53 | PSMA3; ANAPC10 |
| Cdh1-APC/C | | | | | |
| Caspase cascade in apoptosis | 2/59 | 2.00E−01 | 6.04E−01 | 2.48 | CASP4; BCL2L1 |
| CD40L signaling pathway | 1/16 | 2.02E−01 | 6.04E−01 | 4.71 | TNFAIP3 |
| Control of cell cycle and breast | 1/16 | 2.02E−01 | 6.04E−01 | 4.71 | CCNB1 |
| tumor growth by estrogen- | | | | | |
| responsive protein Efp | | | | | |
| Ghrelin biosynthesis, secretion, | 1/16 | 2.02E−01 | 6.04E−01 | 4.71 | MBOAT4 |
| and deacylation | | | | | |
| GluR2-containing AMPA | 1/16 | 2.02E−01 | 6.04E−01 | 4.71 | AP2B1 |
| receptor trafficking | | | | | |
| Hypoxia-inducible factor in the | 1/16 | 2.02E−01 | 6.04E−01 | 4.71 | VHL |
| cardivascular system | | | | | |
| Inhibition of platelet activation | 1/16 | 2.02E−01 | 6.04E−01 | 4.71 | F2R |
| by aspirin | | | | | |
| Signaling by EGFR in cancer | 3/111 | 2.04E−01 | 6.04E−01 | 1.97 | CDKN1B; SH3KBP1; AP2B1 |
| RAGE pathway | 2/60 | 2.05E−01 | 6.04E−01 | 2.44 | CDKN1B; CCL2 |
| Selenium pathway | 2/60 | 2.05E−01 | 6.04E−01 | 2.44 | PRDX4; CCL2 |
| Ephrin receptor B forward | 2/60 | 2.05E−01 | 6.04E−01 | 2.44 | EFNB2; NCK2 |
| pathway | | | | | |
| Licensing factor removal from | 2/61 | 2.10E−01 | 6.04E−01 | 2.40 | PSMA3; PTTG1 |
| origins | | | | | |
| SUMOylation by RanBP2 | 1/17 | 2.13E−01 | 6.04E−01 | 4.41 | SUMO1 |
| regulates transcriptional | | | | | |
| repression | | | | | |
| G1/S-specific transcription | 1/17 | 2.13E−01 | 6.04E−01 | 4.41 | TFDP1 |
| HIV-1 elongation complex | 1/17 | 2.13E−01 | 6.04E−01 | 4.41 | SSRP1 |
| formation in the absence of | | | | | |
| HIV-1 Tat | | | | | |
| Inflammasomes | 1/17 | 2.13E−01 | 6.04E−01 | 4.41 | BCL2L1 |
| Proteasome degradation | 2/63 | 2.21E−01 | 6.04E−01 | 2.32 | PSMA3; UBA1 |
| CXCR4 signaling pathway | 3/116 | 2.22E−01 | 6.04E−01 | 1.88 | HLA-DRA; ITGA7; GNAI1 |
| Pertussis toxin-insensitive CCR5 | 1/18 | 2.24E−01 | 6.04E−01 | 4.15 | CCL2 |
| signaling in macrophage | | | | | |
| Phosphatidylcholine | 1/18 | 2.24E−01 | 6.04E−01 | 4.15 | LPIN1 |
| biosynthesis | | | | | |
| CDK regulation of DNA | 1/18 | 2.24E−01 | 6.04E−01 | 4.15 | CDKN1B |
| replication | | | | | |
| TNFR2 signaling pathway | 1/18 | 2.24E−01 | 6.04E−01 | 4.15 | TNFAIP3 |
| Transcriptional activity | 1/18 | 2.24E−01 | 6.04E−01 | 4.15 | SUMO1 |
| regulation by PML | | | | | |
| Fatty acyl-CoA biosynthesis | 1/18 | 2.24E−01 | 6.04E−01 | 4.15 | ELOVL5 |
| p53 activity regulation | 3/118 | 2.29E−01 | 6.04E−01 | 1.85 | CCNB1; CCNG2; GADD45G |
| Interleukin-12-mediated | 2/65 | 2.31E−01 | 6.04E−01 | 2.24 | HLA-DRA; GADD45G |
| signaling events | | | | | |
| LPA receptor mediated events | 2/65 | 2.31E−01 | 6.04E01− | 2.24 | MAPT; GNAI1 |
| Signaling by NOTCH | 3/119 | 2.33E−01 | 6.04E−01 | 1.83 | TFDP1; DTX1; RBPJ |
| PTEN-dependent cell cycle | 1/19 | 2.35E−01 | 6.04E−01 | 3.92 | CDKN1B |
| arrest and apoptosis | | | | | |
| Calcineurin in effects in | 1/19 | 2.35E−01 | 6.04E−01 | 3.92 | NFATC3 |
| keratinocyte differentiation | | | | | |
| Cell cycle progression regulation | 1/19 | 2.35E−01 | 6.04E−01 | 3.92 | CCNB1 |
| by PLK3 | | | | | |
| TP53 network | 1/19 | 2.35E−01 | 6.04E−01 | 3.92 | SUMO1 |
| Eicosanoid biosynthesis | 1/19 | 2.35E−01 | 6.04E−01 | 3.92 | PNPLA3 |
| Hypoxic and oxygen | 1/19 | 2.35E−01 | 6.04E−01 | 3.92 | VHL |
| homeostasis regulation of HIF-1- | | | | | |
| alpha | | | | | |
| Inhibition of T cell receptor | 1/19 | 2.35E−01 | 6.04E−01 | 3.92 | HLA-DRA |
| signaling by activated Csk | | | | | |
| Retinoblastoma protein | 2/66 | 2.36E−01 | 6.04E−01 | 2.21 | CDKN1B; TFDP1 |
| regulation | | | | | |
| Thyroid-stimulating hormone | 2/66 | 2.36E−01 | 6.04E−01 | 2.21 | CDKN1B; GNAI1 |
| signaling pathway | | | | | |
| Endochondral ossification | 2/66 | 2.36E−01 | 6.04E−01 | 2.21 | CDKN1C; THRA |
| Interleukin-1 regulation of | 3/120 | 2.37E−01 | 6.04E−01 | 1.81 | TNFAIP3; CCL2; MATN2 |
| extracellular matrix | | | | | |
| Peroxisomal lipid metabolism | 1/20 | 2.46E−01 | 6.04E−01 | 3.72 | AGPS |
| Acute myocardial infarction | 1/20 | 2.46E−01 | 6.04E−01 | 3.72 | F2R |
| Apoptotic signaling in response | 1/20 | 2.46E−01 | 6.04E−01 | 3.72 | BCL2L1 |
| to DNA damage | | | | | |
| Sprouty regulation of tyrosine | 1/20 | 2.46E−01 | 6.04E−01 | 3.72 | SH3KBP1 |
| kinase signals | | | | | |
| N-glycan antennae elongation in | 1/20 | 2.46E−01 | 6.04E−01 | 3.72 | MGAT2 |
| the medial/trans-Golgi | | | | | |
| AMPK signaling | 2/68 | 2.46E−01 | 6.04E−01 | 2.14 | CCNB1; SLC2A4RG |
| Interleukin-1 signaling pathway | 3/125 | 2.55E−01 | 6.04E−01 | 1.74 | TNFAIP3; CCL2; UBE2Z |
| S1P/S1P1 pathway | 1/21 | 2.56E−01 | 6.04E−01 | 3.53 | GNAI1 |
| T cell activation co-stimulatory | 1/21 | 2.56E−01 | 6.04E−01 | 3.53 | HLA-DRA |
| signal | | | | | |
| E-cadherin keratinocyte | 1/21 | 2.56E−01 | 6.04E−01 | 3.53 | JUP |
| pathway | | | | | |
| Inactivation of APC/C via direct | 1/21 | 2.56E−01 | 6.04E−01 | 3.53 | ANAPC10 |
| inhibition of the APC/C complex | | | | | |
| Mitochondrial role in apoptotic | 1/21 | 2.56E−01 | 6.04E−01 | 3.53 | BCL2L1 |
| signaling | | | | | |
| Nicotine activity on | 1/21 | 2.56E−01 | 6.04E−01 | 3.53 | GNAI1 |
| dopaminergic neurons | | | | | |
| Oxidative stress-induced gene | 1/21 | 2.56E−01 | 6.04E−01 | 3.53 | CRYZ |
| expression via Nrf2 | | | | | |
| Signaling events mediated by | 2/70 | 2.57E−01 | 6.04E−01 | 2.08 | MYOF; NCK2 |
| VEGFR1 and VEGFR2 | | | | | |
| Rac1 cell motility signaling | 2/71 | 2.62E−01 | 6.04E−01 | 2.05 | NCF2; IQGAP3 |
| pathway | | | | | |
| Viral myocarditis | 2/71 | 2.62E−01 | 6.04E−01 | 2.05 | HLA-DRA; HLA-DPA1 |
| Mitochondrial pathway of | 2/71 | 2.62E−01 | 6.04E−01 | 2.05 | CAPNS1; BCL2L1 |
| apoptosis: multidomain Bcl-2 | | | | | |
| family | | | | | |
| Nephrin interactions | 1/22 | 2.67E−01 | 6.04E−01 | 3.36 | NCK2 |
| Costimulation by the CD28 | 2/72 | 2.67E−01 | 6.04E−01 | 2.02 | HLA-DRA; HLA-DPA1 |
| family | | | | | |
| Cyclin A-Cdk2-associated events | 2/72 | 2.67E−01 | 6.04E−01 | 2.02 | PSMA3; CDKN1B |
| at S phase entry | | | | | |
| Cell-cell communication | 3/129 | 2.70E−01 | 6.04E−01 | 1.68 | JUP; NCK2; DSCAML1 |
| Signaling by NOTCH1 | 2/73 | 2.72E−01 | 6.04E−01 | 1.99 | DTX1; RBPJ |
| Chronic myeloid leukemia | 2/73 | 2.72E−01 | 6.04E−01 | 1.99 | CDKN1B; BCL2L1 |
| Progesterone-initiated oocyte | 1/23 | 2.77E−01 | 6.04E−01 | 3.21 | CCNB1 |
| maturation | | | | | |
| Signaling by bone | 1/23 | 2.77E−01 | 6.04E−01 | 3.21 | CHRDL1 |
| morphogenetic protein {BMP) | | | | | |
| Th1/Th2 differentiation | 1/23 | 2.77E−01 | 6.04E−01 | 3.21 | HLA-DRA |
| pathway | | | | | |
| Incretin biosynthesis, secretion, | 1/23 | 2.77E−01 | 6.04E−01 | 3.21 | FFAR4 |
| and inactivation | | | | | |
| Mitochondrial fatty acid beta- | 1/23 | 2.77E−01 | 6.04E−01 | 3.21 | EHHADH |
| oxidation | | | | | |
| Mitochondrial pathway of | 1/23 | 2.77E−01 | 6.04E−01 | 3.21 | BCL2L1 |
| apoptosis: antiapoptotic Bcl-2 | | | | | |
| family | | | | | |
| Arrhythmogenic right | 2/75 | 2.83E−01 | 6.04E−01 | 1.94 | JUP; ITGA7 |
| ventricular cardiomyopathy | | | | | |
| (ARVC) | | | | | |
| Glycolysis and gluconeogenesis | 2/75 | 2.83E−01 | 6.04E−01 | 1.94 | ALDH2; ENO3 |
| Adipogenesis | 3/133 | 2.86E−01 | 6.04E−01 | 1.63 | RXRA; PNPLA3; LPIN1 |
| Plexin D1 signaling | 1/24 | 2.87E−01 | 6.04E−01 | 3.07 | ITGA7 |
| Signal transduction of S1P | 1/24 | 2.87E−01 | 6.04E−01 | 3.07 | GNAI1 |
| receptor | | | | | |
| Cholesterol biosynthesis | 1/24 | 2.87E−01 | 6.04E−01 | 3.07 | FDPS |
| Triacylglyceride biosynthesis | 1/24 | 2.87E−01 | 6.04E−01 | 3.07 | AGPS |
| NF-kappaB signaling pathway | 1/24 | 2.87E−01 | 6.04E−01 | 3.07 | TNFAIP3 |
| Amino acid metabolism | 4/195 | 2.92E−01 | 6.04E−01 | 1.48 | GCDH; ALDH6A1; PSMA3; DBT |
| Transcriptional regulation of | 2/77 | 2.93E−01 | 6.04E−01 | 1.88 | RXRA; MED4 |
| white adipocyte differentiation | | | | | |
| Alpha-9 beta-1 integrin pathway | 1/25 | 2.97E−01 | 6.04E−01 | 2.94 | SAT1 |
| C-Myc pathway | 1/25 | 2.97E−01 | 6.04E−01 | 2.94 | TRRAP |
| Cellular response to hypoxia | 1/25 | 2.97E−01 | 6.04E−01 | 2.94 | VHL |
| Ck1/Cdk5 regulation by type 1 | 1/25 | 2.97E−01 | 6.04E−01 | 2.94 | NCS1 |
| glutamate receptors | | | | | |
| GO and early G1 pathway | 1/25 | 2.97E−01 | 6.04E−01 | 2.94 | TFDP1 |
| Apoptosis regulation | 2/78 | 2.98E−01 | 6.04E−01 | 1.86 | PSMA3; CDKN1B |
| Diabetes pathways | 3/137 | 3.01E−01 | 6.04E−01 | 1.58 | CCL2; MBOAT4; CD44 |
| Antigen processing: cross | 2/79 | 3.04E−01 | 6.04E−01 | 1.84 | PSMA3; NCF2 |
| presentation | | | | | |
| Signaling events mediated by | 2/79 | 3.04E−01 | 6.04E−01 | 1.84 | SH3KBP1; NCK2 |
| hepatocyte growth factor | | | | | |
| receptor (c-Met) | | | | | |
| p73 transcription factor network | 2/79 | 3.04E−01 | 6.04E−01 | 1.84 | CCNB1; BCL2L11 |
| Glycerophospholipid | 2/79 | 3.04E−01 | 6.04E−01 | 1.84 | GPAT2; CDS2 |
| metabolism | | | | | |
| Axon guidance | 6/325 | 3.04E−01 | 6.04E−01 | 1.33 | EFNB2; NFATC3; NCK2; NTN4; AP2B1; GNAI1 |
| ALK1 pathway | 1/26 | 3.07E−01 | 6.04E−01 | 2.82 | ACVR1 |
| Ras signaling pathway | 1/26 | 3.07E−01 | 6.04E−01 | 2.82 | BCL2L1 |
| Ascorbate and aldarate | 1/26 | 3.07E−01 | 6.04E−01 | 2.82 | ALDH2 |
| metabolism | | | | | |
| Segmentation clock | 1/26 | 3.07E−01 | 6.04E−01 | 2.82 | RBPJ |
| Selenoamino acid metabolism | 1/26 | 3.07E−01 | 6.04E−01 | 2.82 | AHCYL1 |
| TGF-beta signaling in | 1/26 | 3.07E−01 | 6.04E−01 | 2.82 | ACVR1 |
| development | | | | | |
| Estrogen receptor transcription | 1/26 | 3.07E−01 | 6.04E−01 | 2.82 | DBT |
| factor targets | | | | | |
| YAP1-and WWTR1 (TAZ)- | 1/26 | 3.07E−01 | 6.04E−01 | 2.82 | RXRA |
| stimulated gene expression | | | | | |
| Glycosaminoglycan | 1/26 | 3.07E−01 | 6.04E−01 | 2.82 | GLCE |
| biosynthesis: heparan sulfate | | | | | |
| Integrin family cell surface | 1/26 | 3.07E−01 | 6.04E−01 | 2.82 | ITGA7 |
| interactions | | | | | |
| Interleukin-9 regulation of | 1/26 | 3.07E−01 | 6.04E−01 | 2.82 | CCL2 |
| target genes | | | | | |
| Systemic lupus erythematosus | 3/139 | 3.09E−01 | 6.04E−01 | 1.56 | HLA-DRA; TRIM21; HLA-DPA1 |
| Endocytosis | 4/201 | 3.11E−01 | 6.04E−01 | 1.44 | SH3KBP1; F2R; CHMP4A; AP2B1 |
| Antigen processing and | 2/81 | 3.14E−01 | 6.04E−01 | 1.79 | HLA-DRA; HLA-DPA1 |
| presentation | | | | | |
| EGF/EGFR signaling pathway | 3/141 | 3.16E−01 | 6.04E−01 | 1.54 | SH3KBP1; NCK2; AP2B1 |
| ADP signalling through P2Y | 1/27 | 3.17E−01 | 6.04E−01 | 2.71 | GNAI1 |
| purinoceptor 12 | | | | | |
| Proteins and DNA sequences in | 1/27 | 3.17E−01 | 6.04E−01 | 2.71 | NFATC3 |
| cardicac structures | | | | | |
| RORA activates circadian | 1/27 | 3.17E−01 | 6.04E−01 | 2.71 | RXRA |
| expression | | | | | |
| BAD phosphorylation regulation | 1/27 | 3.17E−01 | 6.04E−01 | 2.71 | BCL2L1 |
| CTCF pathway | 1/27 | 3.17E−01 | 6.04E−01 | 2.71 | CDKN1B |
| Cell cycle: G2/M checkpoint | 1/27 | 3.17E−01 | 6.04E−01 | 2.71 | CCNB1 |
| Control of gene expression by | 1/27 | 3.17E−01 | 6.04E−01 | 2.71 | RXRA |
| vitamin D receptor | | | | | |
| Cooperation of prefoldin and | 1/27 | 3.17E−01 | 6.04E−01 | 2.71 | PFDN6 |
| TriC/CCT in actin and tubulin | | | | | |
| folding | | | | | |
| Glycolysis | 1/27 | 3.17E−01 | 6.04E−01 | 2.71 | ENO3 |
| Insulin regulation of blood | 1/27 | 3.17E−01 | 6.04E−01 | 2.71 | TBC1D4 |
| glucose | | | | | |
| Mammalian calpain regulation | 1/27 | 3.17E−01 | 6.04E−01 | 2.71 | CAPNS1 |
| of cell motility | | | | | |
| T cell signal transduction | 2/83 | 3.24E−01 | 6.04E−01 | 1.74 | NCK2; NFATC3 |
| Meiosis | 2/83 | 3.24E−01 | 6.04E−01 | 1.74 | STAG1; REC8 |
| Phosphatidic acid biosynthesis | 1/28 | 3.26E−01 | 6.04E−01 | 2.61 | GPAT2 |
| Recycling pathway of cell | 1/28 | 3.26E−01 | 6.04E−01 | 2.61 | AP2B1 |
| adhesion molecule L1 | | | | | |
| Endosomal sorting complex | 1/28 | 3.26E−01 | 6.04E−01 | 2.61 | CHMP4A |
| required for transport (ESCRT) | | | | | |
| pathway | | | | | |
| G-protein activation | 1/28 | 3.26E−01 | 6.04E−01 | 2.61 | GNAI1 |
| Multi-step regulation of | 1/28 | 3.26E−01 | 6.04E−01 | 2.61 | TRRAP |
| transcription by PITX2 | | | | | |
| Nef in HIV-1 replication and | 1/28 | 3.26E−01 | 6.04E−01 | 2.61 | AP2B1 |
| disease pathogenesis | | | | | |
| PLipidK-regulated gene | 1/28 | 3.26E−01 | 6.04E−01 | 2.61 | CCL2 |
| expression | | | | | |
| Interleukin-5 regulation of | 3/144 | 3.28E−01 | 6.04E−01 | 1.50 | CASP4; CCL2; CD44 |
| apoptosis | | | | | |
| ECM-receptor interaction | 2/84 | 3.29E−01 | 6.04E−01 | 1.72 | ITGA7; CD44 |
| Asparagine N-linked | 2/85 | 3.34E−01 | 6.04E−01 | 1.70 | MGAT2; DDOST |
| glycosylation | | | | | |
| C-Myb transcription factor | 2/85 | 3.34E−01 | 6.04E−01 | 1.70 | CCNB1; CDKN1B |
| network | | | | | |
| TGF-beta regulation of skeletal | 2/85 | 3.34E−01 | 6.04E−01 | 1.70 | ACVR1; TFDP1 |
| system development | | | | | |
| Myc active pathway | 2/85 | 3.34E−01 | 6.04E−01 | 1.70 | CCNB1; TRRAP |
| Adherens junction actin | 1/29 | 3.36E−01 | 6.04E−01 | 2.52 | JUP |
| cytoskeletal organization | | | | | |
| BARD1 signaling events | 1/29 | 3.36E−01 | 6.04E−01 | 2.52 | BARD1 |
| S1P/S1P3 pathway | 1/29 | 3.36E−01 | 6.04E−01 | 2.52 | GNAI1 |
| T cell receptor calcium pathway | 1/29 | 3.36E−01 | 6.04E−01 | 2.52 | NFATC3 |
| Thyroid cancer | 1/29 | 3.36E−01 | 6.04E−01 | 2.52 | RXRA |
| Histidine metabolism | 1/29 | 3.36E−01 | 6.04E−01 | 2.52 | ALDH2 |
| Homologous recombination | 1/29 | 3.36E−01 | 6.04E−01 | 2.52 | EME1 |
| IGF1 pathway | 1/29 | 3.36E−01 | 6.04E−01 | 2.52 | NCK2 |
| Inhibition of insulin secretion by | 1/29 | 3.36E−01 | 6.04E−01 | 2.52 | GNAI1 |
| adrenaline/noradrenaline | | | | | |
| T cell receptor regulation of | 10/603 | 3.38E−01 | 6.04E−01 | 1.19 | BARD1; FDPS; CCNB1; AHCYL1; PRDX4; CDKN1B; F2R; |
| apoptosis | | | | | CASP4; TNFAIP3; GNAI1 |
| Mitotic G2-G2/M phases | 2/87 | 3.45E−01 | 6.04E−01 | 1.66 | CCNB1; NEK2 |
| Retinoic acid receptor-mediated | 1/30 | 3.45E−01 | 6.04E−01 | 2.43 | RXRA |
| signaling | | | | | |
| Signaling pathway from | 1/30 | 3.45E−01 | 6.04E−01 | 2.43 | NFATC3 |
| G-protein families | | | | | |
| Tumor necrosis factor (TNF) | 1/30 | 3.45E−01 | 6.04E−01 | 2.43 | TNFAIP3 |
| pathway | | | | | |
| Glutamate binding and | 1/30 | 3.45E−01 | 6.04E−01 | 2.43 | AP2B1 |
| activation of AMPA receptors | | | | | |
| and synaptic plasticity | | | | | |
| Heparan sulfate/heparin | 1/30 | 3.45E−01 | 6.04E−01 | 2.43 | GLCE |
| glycosaminoglycan (HS-GAG) | | | | | |
| biosynthesis | | | | | |
| Interleukin-2/STAT5 pathway | 1/30 | 3.45E−01 | 6.04E−01 | 2.43 | BCL2L1 |
| Ovarian infertility genes | 1/30 | 3.45E−01 | 6.04E−01 | 2.43 | CDKN1B |
| DNA replication pre-Initiation | 2/88 | 3.50E−01 | 6.04E−01 | 1.64 | PSMA3; CDKN1B |
| HIF-1 degradation in normoxia | 2/88 | 3.50E−01 | 6.04E−01 | 1.64 | PSMA3; VHL |
| Hematopoietic cell lineage | 2/88 | 3.50E−01 | 6.04E−01 | 1.64 | HLA-DRA; CD44 |
| Activated NOTCH1 signaling in | 1/31 | 3.54E−01 | 6.04E−01 | 2.35 | DTX1 |
| the nucleus | | | | | |
| Alpha-V beta-3 integrin/OPN | 1/31 | 3.54E−01 | 6.04E−01 | 2.35 | CD44 |
| pathway | | | | | |
| Signal amplification | 1/31 | 3.54E−01 | 6.04E−01 | 2.35 | GNAI1 |
| Stathmin and breast cancer | 1/31 | 3.54E−01 | 6.04E−01 | 2.35 | CCNB1 |
| resistance to antimicrotubule | | | | | |
| agents | | | | | |
| Transport of vitamins, | 1/31 | 3.54E−01 | 6.04E−01 | 2.35 | SLC35A1 |
| nucleosides, and related | | | | | |
| molecules | | | | | |
| Gluconeogenesis | 1/31 | 3.54E−01 | 6.04E−01 | 2.35 | ENO3 |
| Signaling by the B cell receptor | 3/151 | 3.54E−01 | 6.04E−01 | 1.43 | PSMA3; CDKN1B; SH3KBP1 |
| (BCR) | | | | | |
| Immune system signaling by | 5/280 | 3.55E−01 | 6.04E−01 | 1.29 | SUMO1; MX2; HLA-DRA; CD44; HLA-DPA1 |
| interferons, interleukins, | | | | | |
| prolactin, and growth hormones | | | | | |
| Pancreatic beta-cell | 1/32 | 3.63E−01 | 6.09E−01 | 2.28 | RBPJ |
| development regulation | | | | | |
| Prothrombin activation intrinsic | 1/32 | 3.63E−01 | 6.09E−01 | 2.28 | F2R |
| pathway | | | | | |
| Serotonin HTR1 group and FOS | 1/32 | 3.63E−01 | 6.09E−01 | 2.28 | GNAI1 |
| pathway | | | | | |
| Signaling by Robo receptor | 1/32 | 3.63E−01 | 6.09E−01 | 2.28 | NCK2 |
| Sphingolipid de novo | 1/32 | 3.63E−01 | 6.09E−01 | 2.28 | VAPB |
| biosynthesis | | | | | |
| Negative regulators of RIG-I/ | 1/33 | 3.72E−01 | 6.23E−01 | 2.21 | TNFAIP3 |
| MDA5 signaling | | | | | |
| Cysteine and methionine | 1/34 | 3.81E−01 | 6.30E−01 | 2.14 | AHCYL1 |
| metabolism | | | | | |
| Fatty acid beta oxidation | 1/34 | 3.81E−01 | 6.30E−01 | 2.14 | GCDH |
| HIF-2-alpha transcription factor | 1/34 | 3.81E−01 | 6.30E−01 | 2.14 | VHL |
| network | | | | | |
| N-cadherin signaling events | 1/34 | 3.81E−01 | 6.30E−01 | 2.14 | JUP |
| Toll receptor cascades | 3/159 | 3.85E−01 | 6.31E−01 | 1.36 | TLR9; TNFAIP3; UBE2Z |
| Signal transduction by L1 | 1/35 | 3.90E−01 | 6.31E−01 | 2.08 | AP2B1 |
| Cytokines and inflammatory | 1/35 | 3.90E−01 | 6.31E−01 | 2.08 | HLA-DRA |
| response | | | | | |
| Ether lipid metabolism | 1/35 | 3.90E−01 | 6.31E−01 | 2.08 | AGPS |
| Integrated cancer pathway | 1/35 | 3.90E−01 | 6.31E−01 | 2.08 | BARD1 |
| Interleukin-2/PI3K pathway | 1/35 | 3.90E−01 | 6.31E−01 | 2.08 | BCL2L1 |
| Linoleic acid metabolism | 1/35 | 3.90E−01 | 6.31E−01 | 2.08 | ELOVL5 |
| Actin cytoskeleton regulation | 4/226 | 3.90E−01 | 6.31E−01 | 1.27 | MYL3; F2R; ITGA7; IQGAP3 |
| TSH regulation of gene | 2/97 | 3.95E−01 | 6.35E−01 | 1.49 | TNFAIP3; CCL2 |
| expression | | | | | |
| Mitochondrial pathway of | 2/97 | 3.95E−01 | 6.35E−01 | 1.49 | BCL2L11; CAPNS1 |
| apoptosis: BH3-only Bcl-2 family | | | | | |
| Phospholipids as signaling | 1/36 | 3.98E−01 | 6.36E−01 | 2.02 | GNAI1 |
| intermediaries | | | | | |
| Transport to the Golgi and | 1/36 | 3.98E−01 | 6.36E−01 | 2.02 | MGAT2 |
| subsequent modification | | | | | |
| GM-CSF-mediated signaling | 1/36 | 3.98E−01 | 6.36E−01 | 2.02 | CCL2 |
| events | | | | | |
| Downstream signaling events Of | 2/98 | 4.00E−01 | 6.36E−01 | 1.47 | PSMA3; CDKN1B |
| B cell receptor (BCR) | | | | | |
| ALK in cardiac myocytes | 1/37 | 4.07E−01 | 6.41E−01 | 1.96 | ACVR1 |
| Interleukin-23-mediated | 1/37 | 4.07E−01 | 6.41E−01 | 1.96 | CCL2 |
| signaling events | | | | | |
| Latent infection of Homo | 1/37 | 4.07E−01 | 6.41E−01 | 1.96 | NCF2 |
| sapiens with Mycobacterium | | | | | |
| tuberculosis | | | | | |
| PI3K/AKT activation | 1/37 | 4.07E−01 | 6.41E−01 | 1.96 | CDKN1B |
| Dilated cardiomyopathy | 2/100 | 4.09E−01 | 6.42E−01 | 1.44 | MYL3; ITGA7 |
| Integrin-mediated cell adhesion | 2/100 | 4.09E−01 | 6.42E−01 | 1.44 | CAPNS1; ITGA7 |
| TNF-alpha signaling pathway | 2/101 | 4.14E−01 | 6.44E−01 | 1.43 | TNFAIP3; BCL2L1 |
| BMAL1-CLOCK/NPAS2 activates | 1/38 | 4.15E−01 | 6.44E−01 | 1.91 | RXRA |
| circadian expression | | | | | |
| Striated muscle contraction | 1/38 | 4.15E−01 | 6.44E−01 | 1.91 | MYL3 |
| p38 alpha/beta MAPK | 1/38 | 4.15E−01 | 6.44E−01 | 1.91 | MAPKAPK5 |
| downstream pathway | | | | | |
| E-cadherin nascent AJ-like | 1/39 | 4.23E−01 | 6.49E−01 | 1.86 | JUP |
| junctions pathway | | | | | |
| Vitamin A and carotenoid | 1/39 | 4.23E−01 | 6.49E−01 | 1.86 | RXRA |
| metabolism | | | | | |
| G13 signaling pathway | 1/39 | 4.23E−01 | 6.49E−01 | 1.86 | CIT |
| GAB1 signalosome | 1/39 | 4.23E−01 | 6.49E−01 | 1.86 | CDKN1B |
| Chromosome maintenance | 2/103 | 4.24E−01 | 6.49E−01 | 1.40 | STAG1; REC8 |
| Electron transport chain | 2/105 | 4.34E−01 | 6.62E−01 | 1.37 | SCO1; COX17 |
| Plasma membrane estrogen | 1/41 | 4.39E−01 | 6.62E−01 | 1.76 | GNAI1 |
| receptor signaling | | | | | |
| Transcriptional activity of | 1/41 | 4.39E−01 | 6.62E−01 | 1.76 | TFDP1 |
| SMAD2/SMAD3-SMAD4 | | | | | |
| heterotrimer | | | | | |
| LipidBB1 internalization | 1/41 | 4.39E−01 | 6.62E−01 | 1.76 | SH3KBP1 |
| pathway | | | | | |
| Fc epsilon receptor I signaling in | 1/41 | 4.39E−01 | 6.62E−01 | 1.76 | NFATC3 |
| mast cells | | | | | |
| Insulin biosynthesis and | 1/41 | 4.39E−01 | 6.62E−01 | 1.76 | DDOST |
| processing | | | | | |
| BMP receptor signaling | 1/42 | 4.47E−01 | 6.67E−01 | 1.72 | CHRDL1 |
| TWEAK signaling pathway | 1/42 | 4.47E−01 | 6.67E−01 | 1.72 | CCL2 |
| Messenger RNA splicing: minor | 1/42 | 4.47E−01 | 6.67E−01 | 1.72 | SF3B1 |
| pathway | | | | | |
| Netrin-1 signaling | 1/42 | 4.47E−01 | 6.67E−01 | 1.72 | NTN4 |
| Voltage-gated potassium | 1/43 | 4.55E−01 | 6.72E−01 | 1.68 | KCNS2 |
| channels | | | | | |
| G2/M checkpoints | 1/43 | 4.55E−01 | 6.72E−01 | 1.68 | CCNB1 |
| Glycosaminoglycan metabolism | 2/110 | 4.57E−01 | 6.72E−01 | 1.31 | GLCE; CD44 |
| Leptin influence on immune | 2/110 | 4.57E−01 | 6.72E−01 | 1.31 | NCF2; CCL2 |
| response | | | | | |
| Vasopressin-regulated water | 1/44 | 4.63E−01 | 6.72E−01 | 1.64 | DYNLL2 |
| reabsorption | | | | | |
| G-protein-mediated events | 1/44 | 4.63E−01 | 6.72E−01 | 1.64 | GNAI1 |
| Gastrin pathway | 1/44 | 4.63E−01 | 6.72E−01 | 1.64 | CD44 |
| HNF3A pathway | 1/44 | 4.63E−01 | 6.72E−01 | 1.64 | CDKN1B |
| Heart development | 1/44 | 4.63E−01 | 6.72E−01 | 1.64 | NFATC3 |
| PI3K events in LipidBB2 signaling | 1/44 | 4.63E−01 | 6.72E−01 | 1.64 | CDKN1B |
| Innate immune system | 5/319 | 4.63E−01 | 6.72E−01 | 1.12 | CASP4; TLR9; TNFAIP3; DDOST; BCL2L1 |
| S phase | 2/112 | 4.66E−01 | 6.73E−01 | 1.28 | PSMA3; CDKN1B |
| Lipid metabolism regulation by | 2/112 | 4.66E−01 | 6.73E−01 | 1.28 | RXRA; MED4 |
| peroxisome proliferator- | | | | | |
| activated receptor alpha | | | | | |
| (PPAR-alpha) | | | | | |
| G alpha (z) signaling events | 1/45 | 4.70E−01 | 6.73E−01 | 1.60 | GNAI1 |
| Integrin-linked kinase signaling | 1/45 | 4.70E−01 | 6.73E−01 | 1.60 | NCK2 |
| Interleukin-12/STAT4 pathway | 1/45 | 4.70E−01 | 6.73E−01 | 1.60 | HLA-DRA |
| Interleukin-3 signaling pathway | 1/45 | 4.70E−01 | 6.73E−01 | 1.60 | BCL2L1 |
| Polo-like kinase 1 (PLK1) | 1/46 | 4.78E−01 | 6.79E−01 | 1.57 | CCNB1 |
| pathway | | | | | |
| Alpha-6 beta-1 and alpha-6 | 1/46 | 4.78E−01 | 6.79E−01 | 1.57 | RXRA |
| beta-4 integrin signaling | | | | | |
| RhoA activity regulation | 1/46 | 4.78E−01 | 6.79E−01 | 1.57 | CDKN1B |
| Pathways in cancer | 5/325 | 4.79E−01 | 6.79E−01 | 1.10 | CDKN1B; RXRA; JUP; VHL; BCL2L1 |
| TGF-beta signaling pathway | 3/185 | 4.81E−01 | 6.80E−01 | 1.16 | TFDP1; SUMO1; PJA1 |
| Regulation of NFAT | 1/47 | 4.85E−01 | 6.81E−01 | 1.53 | NFATC3 |
| transcription factors | | | | | |
| Regulation of transcription by | 1/47 | 4.85E−01 | 6.81E−01 | 1.53 | RBPJ |
| NOTCH1 intracellular domain | | | | | |
| LKB1 signaling events | 1/47 | 4.85E−01 | 6.81E−01 | 1.53 | MAPT |
| Leukocyte transendothelial | 2/117 | 4.89E−01 | 6.85E−01 | 1.23 | NCF2; GNAI1 |
| migration | | | | | |
| Post-translational regulation of | 1/48 | 4.92E−01 | 6.85E−01 | 1.50 | JUP |
| adherens junction stability and | | | | | |
| disassembly | | | | | |
| Diurnally regulated genes with | 1/48 | 4.92E−01 | 6.85E−01 | 1.50 | SUMO1 |
| circadian orthologs | | | | | |
| Energy metabolism | 1/48 | 4.92E−01 | 6.85E−01 | 1.50 | RXRA |
| Prolactin regulation of apoptosis | 2/118 | 4.94E−01 | 6.86E−01 | 1.22 | BCL6; MATN2 |
| Transmission across chemical | 3/190 | 4.98E−01 | 6.88E−01 | 1.13 | ALDH2; AP2B1; GNAI1 |
| synapses | | | | | |
| Arf6 integrin-mediated signaling | 1/49 | 4.99E−01 | 6.88E−01 | 1.47 | ITGA7 |
| pathway | | | | | |
| Muscle contraction | 1/49 | 4.99E−01 | 6.88E−01 | 1.47 | MYL3 |
| Translation factors | 1/50 | 5.06E−01 | 6.96E−01 | 1.44 | EIF2B2 |
| Signaling by PDGF | 2/122 | 5.11E−01 | 7.01E−01 | 1.18 | CDKN1B; NCK2 |
| Type I interferon (interferon- | 1/51 | 5.13E−01 | 7.01E−01 | 1.41 | PTPN9 |
| alpha/beta) pathway | | | | | |
| Metabolism of vitamins and | 1/51 | 5.13E−01 | 7.01E−01 | 1.41 | CYB5A |
| cofactors | | | | | |
| Interleukin-4 regulation of | 4/267 | 5.16E−01 | 7.03E−01 | 1.07 | BCL2L11; BCL6; CCL2; GNAI1 |
| apoptosis | | | | | |
| Apoptotic execution phase | 1/52 | 5.20E−01 | 7.04E−01 | 1.38 | MAPT |
| Vitamin B12 metabolism | 1/52 | 5.20E−01 | 7.04E−01 | 1.38 | CCL2 |
| Heparan sulfate/heparin | 1/52 | 5.20E−01 | 7.04E−01 | 1.38 | GLCE |
| glycosaminoglycan (HS-GAG) | | | | | |
| metabolism | | | | | |
| Integration of energy | 2/125 | 5.24E−01 | 7.04E−01 | 1.15 | FFAR4; GNAI1 |
| metabolism | | | | | |
| Interleukin-2 signaling pathway | 12/847 | 5.25E−01 | 7.04E−01 | 1.01 | SNX4; CYB5A; CDKN1B; BCL6; CCNG2; NFATC3; PTPN9; |
| | | | | | MAPT; BRD8; TRIM21; GADD45G; BCL2L1 |
| Protein folding | 1/53 | 5.27E−01 | 7.04E−01 | 1.36 | PFDN6 |
| Amyotrophic lateral sclerosis | 1/53 | 5.27E−01 | 7.04E−01 | 1.36 | BCL2L1 |
| (ALS) | | | | | |
| GABA A and B receptor | 1/53 | 5.27E−01 | 7.04E−01 | 1.36 | GNAI1 |
| activation | | | | | |
| JNK/MAPK pathway | 1/53 | 5.27E−01 | 7.04E−01 | 1.36 | NFATC3 |
| Jak-STAT signaling pathway | 3/199 | 5.29E−01 | 7.05E−01 | 1.08 | PTPN9; OSMR; BCL2L1 |
| HIV infection | 3/200 | 5.33E−01 | 7.05E−01 | 1.07 | PSMA3; AP2B1; SSRP1 |
| Binding of chemokines to | 1/54 | 5.33E−01 | 7.05E−01 | 1.33 | CCL2 |
| chemokine receptors | | | | | |
| Kit receptor signaling pathway | 1/54 | 5.33E−01 | 7.05E−01 | 1.33 | MAPT |
| Non-small cell lung cancer | 1/54 | 5.33E−01 | 7.05E−01 | 1.33 | RXRA |
| HIV factor interactions with host | 2/128 | 5.37E−01 | 7.07E−01 | 1.12 | PSMA3; AP2B1 |
| Developmental biology | 6/420 | 5.37E−01 | 7.07E−01 | 1.02 | RXRA; NCK2; NTN4; AP2B1; RBPJ; MED4 |
| TAp63 pathway | 1/55 | 5.40E−01 | 7.09E−01 | 1.31 | SSRP1 |
| Pathogenic Escherichia coli | 1/57 | 5.53E−01 | 7.17E−01 | 1.26 | NCK2 |
| infection | | | | | |
| Acute myeloid leukemia | 1/57 | 5.53E−01 | 7.17E−01 | 1.26 | JUP |
| SHP2 signaling | 1/57 | 5.53E−01 | 7.17E−01 | 1.26 | GNAI1 |
| Inositol phosphate metabolism | 1/57 | 5.53E−01 | 7.17E−01 | 1.26 | ALDH6A1 |
| PI3K cascade | 1/57 | 5.53E−01 | 7.17E−01 | 1.26 | CDKN1B |
| DNA replication | 3/207 | 5.56E−01 | 7.20E−01 | 1.04 | STAG1; PSMA3; CDKN1B |
| Cell adhesion molecules (CAMs) | 2/133 | 5.58E−01 | 7.21E−01 | 1.08 | HLA-DRA; HLA-DPA1 |
| Neuronal system | 4/283 | 5.62E−01 | 7.24E−01 | 1.01 | ALDH2; KCNS2; AP2B1; GNAI1 |
| ATF2 transcription factor | 1/59 | 5.65E−01 | 7.26E−01 | 1.22 | BCL2L1 |
| network | | | | | |
| RNA degradation | 1/59 | 5.65E−01 | 7.26E−01 | 1.22 | ENO3 |
| NGF signaling via TRKA from the | 2/136 | 5.70E−01 | 7.30E−01 | 1.05 | CDKN1B; AP2B1 |
| plasma membrane | | | | | |
| Cytochrome P450 pathway | 1/61 | 5.77E−01 | 7.33E−01 | 1.17 | CYB5A |
| Glucose metabolism | 1/61 | 5.77E−01 | 7.33E−01 | 1.17 | ENO3 |
| HIV genome transcription | 1/61 | 5.77E−01 | 7.33E−01 | 1.17 | SSRP1 |
| Leptin signaling pathway | 1/61 | 5.77E−01 | 7.33E−01 | 1.17 | BCL2L1 |
| Shigellosis | 1/62 | 5.83E−01 | 7.36E−01 | 1.16 | CD44 |
| Signaling events mediated by | 1/62 | 5.83E−01 | 7.36E−01 | 1.16 | NCK2 |
| focal adhesion kinase | | | | | |
| Circadian rhythm | 1/62 | 5.83E−01 | 7.36E−01 | 1.16 | RXRA |
| Protein metabolism | 6/442 | 5.87E−01 | 7.40E−01 | 0.97 | EIF2B2; COX17; TIMM22; MGAT2; DDOST; PFDN6 |
| Folate metabolism | 1/63 | 5.89E−01 | 7.40E−01 | 1.14 | CCL2 |
| Toll-like receptor signaling | 2/142 | 5.94E−01 | 7.40E−01 | 1.01 | TLR9; TNFAIP3 |
| pathway regulation | | | | | |
| Ubiquitin-mediated degradation | 1/64 | 5.95E−01 | 7.40E−01 | 1.12 | PSMA3 |
| of phosphorylated Cdc25A | | | | | |
| Endothelins | 1/64 | 5.95E−01 | 7.40E−01 | 1.12 | GNAI1 |
| Interferon alpha/beta signaling | 1/64 | 5.95E−01 | 7.40E−01 | 1.12 | MX2 |
| MAPK cascade role in | 1/64 | 5.95E−01 | 7.40E−01 | 1.12 | VHL |
| angiogenesis | | | | | |
| Signaling by NGF | 3/221 | 6.01E−01 | 7.44E−01 | 0.97 | CDKN1B; BCL2L11; AP2B1 |
| Disease | 9/674 | 6.05E−01 | 7.44E−01 | 0.95 | PSMA3; CDKN1B; SH3KBP1; NCF2; CCL2; AP2B1; SSRP1; |
| | | | | | MBOAT4; CD44 |
| Beta-1 integrin cell surface | 1/66 | 6.06E−01 | 7.44E−01 | 1.08 | ITGA7 |
| interactions | | | | | |
| Signaling by TGF-beta receptor | 1/66 | 6.06E−01 | 7.44E−01 | 1.08 | TFDP1 |
| complex | | | | | |
| Destabilization of mRNA by | 1/66 | 6.06E−01 | 7.44E−01 | 1.08 | PSMA3 |
| AUF1 (hnRNP DO) | | | | | |
| Adipocytokine signaling | 1/67 | 6.12E−01 | 7.44E−01 | 1.07 | RXRA |
| pathway | | | | | |
| SIDS susceptibility pathways | 1/67 | 6.12E−01 | 7.44E−01 | 1.07 | CDCA7L |
| T cell receptor signaling in naive | 1/67 | 6.12E−01 | 7.44E−01 | 1.07 | HLA-DRA |
| CD4+ T cells | | | | | |
| Telomerase regulation | 1/67 | 6.12E−01 | 7.44E−01 | 1.07 | CDKN1B |
| G alpha 13 pathway | 1/67 | 6.12E−01 | 7.44E−01 | 1.07 | SLC9B2 |
| Activation of NF-kappaB in B | 1/68 | 6.17E−01 | 7.44E−01 | 1.05 | PSMA3 |
| cells | | | | | |
| CD8/T cell receptor downstream | 1/68 | 6.17E−01 | 7.44E−01 | 1.05 | NFATC3 |
| pathway | | | | | |
| Phase I of biological oxidations: | 1/69 | 6.23E−01 | 7.44E−01 | 1.04 | ALDH2 |
| functionalization of compounds | | | | | |
| NFAT involvement in | 1/69 | 6.23E−01 | 7.44E−01 | 1.04 | NFATC3 |
| hypertrophy of the heart | | | | | |
| Translation | 2/151 | 6.27E−01 | 7.44E−01 | 0.94 | EIF2B2; DDOST |
| Pancreatic cancer | 1/70 | 6.28E−01 | 7.44E−01 | 1.02 | BCL2L1 |
| Antiviral mechanism by | 1/70 | 6.28E−01 | 7.44E−01 | 1.02 | MX2 |
| interferon-stimulated genes | | | | | |
| Renal cell carcinoma | 1/70 | 6.28E−01 | 7.44E−01 | 1.02 | VHL |
| CDC42 signaling events | 1/70 | 6.28E−01 | 7.44E−01 | 1.02 | IQGAP3 |
| Signaling events mediated by | 1/70 | 6.28E−01 | 7.44E−01 | 1.02 | SUMO1 |
| HDAC class I | | | | | |
| Complement and coagulation | 1/70 | 6.28E−01 | 7.44E−01 | 1.02 | F2R |
| cascades | | | | | |
| G alpha q pathway | 1/70 | 6.28E−01 | 7.44E−01 | 1.02 | SLC9B2 |
| Long-term depression | 1/70 | 6.28E−01 | 7.44E−01 | 1.02 | GNAI1 |
| Nucleotide metabolism | 1/70 | 6.28E−01 | 7.44E−01 | 1.02 | DHODH |
| EGFR1 pathway | 2/152 | 6.31E−01 | 7.44E−01 | 0.94 | BCL6; MAPT |
| Integrated breast cancer | 2/152 | 6.31E−01 | 7.44E−01 | 0.94 | BARD1; DHTKD1 |
| pathway | | | | | |
| Interleukin-6 signaling pathway | 1/71 | 6.33E−01 | 7.46E−01 | 1.01 | BCL2L1 |
| Sphingolipid metabolism | 1/72 | 6.38E−01 | 7.49E−01 | 0.99 | VAPB |
| Myocyte adrenergic pathway | 1/72 | 6.38E−01 | 7.49E−01 | 0.99 | GNAI1 |
| Carbohydrate metabolism | 3/235 | 6.42E−01 | 7.50E−01 | 0.91 | GLCE; ENO3; CD44 |
| Degradation of beta-catenin by | 1/73 | 6.43E−01 | 7.50E−01 | 0.98 | PSMA3 |
| the destruction complex | | | | | |
| Myc repressed pathway | 1/73 | 6.43E−01 | 7.50E−01 | 0.98 | CDKN1B |
| Wnt/calcium/cyclic GMP | 1/74 | 6.48E−01 | 7.52E−01 | 0.96 | NFATC3 |
| pathway | | | | | |
| Gastric acid secretion | 1/74 | 6.48E−01 | 7.52E−01 | 0.96 | GNAI1 |
| Integrins in angiogenesis | 1/74 | 6.48E−01 | 7.52E−01 | 0.96 | CDKN1B |
| RIG-I/MDA5-mediated induction | 1/76 | 6.58E−01 | 7.60E−01 | 0.94 | TNFAIP3 |
| of interferon-alpha/beta | | | | | |
| pathways | | | | | |
| VEGF signaling pathway | 1/76 | 6.58E−01 | 7.60E−01 | 0.94 | NFATC3 |
| Mitochondrial pathway of | 1/77 | 6.63E−01 | 7.64E−01 | 0.93 | CAPNS1 |
| apoptosis: caspases | | | | | |
| Phosphatidylinositol signaling | 1/78 | 6.68E−01 | 7.67E−01 | 0.91 | CDS2 |
| system | | | | | |
| Signaling by SCF-KIT | 1/78 | 6.68E−01 | 7.67E−01 | 0.91 | CDKN1B |
| Unfolded protein response | 1/79 | 6.72E−01 | 7.71E−01 | 0.90 | CCL2 |
| Cardiac muscle contraction | 1/80 | 6.77E−01 | 7.71E−01 | 0.89 | MYL3 |
| Nuclear beta-catenin signaling | 1/80 | 6.77E−01 | 7.71E−01 | 0.89 | TRRAP |
| and target gene transcription | | | | | |
| regulation | | | | | |
| Opioid signaling | 1/80 | 6.77E−01 | 7.71E−01 | 0.89 | GNAI1 |
| Protein processing in the | 2/166 | 6.78E−01 | 7.71E−01 | 0.86 | ATXN3; DDOST |
| endoplasmic reticulum | | | | | |
| MAP kinase signaling pathway | 1/81 | 6.82E−01 | 7.74E−01 | 0.88 | MAPKAPK5 |
| SMAD2/3 nuclear pathway | 1/82 | 6.86E−01 | 7.77E−01 | 0.87 | TFDP1 |
| Cell junction organization | 1/84 | 6.95E−01 | 7.86E−01 | 0.85 | JUP |
| Integrin cell surface interactions | 1/85 | 6.99E−01 | 7.89E−01 | 0.84 | ITGA7 |
| mRNA stability regulation by | 1/86 | 7.03E−01 | 7.91E−01 | 0.83 | PSMA3 |
| proteins that bind AU-rich | | | | | |
| elements | | | | | |
| Non-class A, B, C GPCRs | 1/86 | 7.03E−01 | 7.91E−01 | 0.83 | F2R |
| Androgen receptor signaling, | 1/88 | 7.12E−01 | 7.99E−01 | 0.81 | SUMO1 |
| proteolysis, and transcription | | | | | |
| regulation | | | | | |
| FSH regulation of apoptosis | 3/263 | 7.15E−01 | 8.00E−01 | 0.81 | CDKN1C; CASP4; ZBED1 |
| Prostate cancer | 1/89 | 7.16E−01 | 8.00E−01 | 0.80 | CDKN1B |
| Thymic stromal lymphopoietin | 1/90 | 7.20E−01 | 8.01E−01 | 0.79 | CCL2 |
| (TSLP) pathway | | | | | |
| Gap junction pathway | 1/90 | 7.20E−01 | 8.01E−01 | 0.79 | GNAI1 |
| Cytokine-cytokine receptor | 3/265 | 7.20E−01 | 8.01E−01 | 0.80 | ACVR1; CCL2; OSMR |
| interaction | | | | | |
| Neural crest differentiation | 1/91 | 7.24E−01 | 8.03E−01 | 0.78 | RBPJ |
| Signaling by LipidBB4 | 1/93 | 7.31E−01 | 8.10E−01 | 0.76 | CDKN1B |
| L1CAM interactions | 1/94 | 7.35E−01 | 8.13E−01 | 0.76 | AP2B1 |
| Transport of inorganic | 1/95 | 7.39E−01 | 8.14E−01 | 0.75 | SLC4A8 |
| cations/anions and amino | | | | | |
| acids/oligopeptides | | | | | |
| G-protein signaling pathways | 1/95 | 7.39E−01 | 8.14E−01 | 0.75 | GNAI1 |
| M phase pathway | 1/96 | 7.43E−01 | 8.16E−01 | 0.74 | STAG1 |
| Chemokine signaling pathway | 2/189 | 7.45E−01 | 8.18E−01 | 0.75 | CCL2; GNAI1 |
| Insulin signaling pathway | 3/277 | 7.48E−01 | 8.19E−01 | 0.77 | EIF2B2; TBC1D4; NCK2 |
| Wnt signaling pathway and | 1/98 | 7.50E−01 | 8.19E−01 | 0.73 | CD44 |
| pluripotency | | | | | |
| Peptide G-protein coupled | 2/192 | 7.53E−01 | 8.19E−01 | 0.74 | F2R; CCL2 |
| receptors | | | | | |
| Potassium channels | 1/99 | 7.53E−01 | 8.19E−01 | 0.72 | KCNS2 |
| Granule cell survival pathway | 1/99 | 7.53E−01 | 8.19E−01 | 0.72 | MAPT |
| Pyrimidine metabolism | 1/100 | 7.57E−01 | 8.21E−01 | 0.71 | DHODH |
| RNA polymerase II transcription | 1/101 | 7.60E−01 | 8.22E−01 | 0.70 | SSRP1 |
| Melanogenesis | 1/101 | 7.60E−01 | 8.22E−01 | 0.70 | GNAI1 |
| Post-translational protein | 2/196 | 7.63E−01 | 8.23E−01 | 0.72 | MGAT2; DDOST |
| modification | | | | | |
| Signaling by LipidBB2 | 1/102 | 7.63E−01 | 8.23E−01 | 0.70 | CDKN1B |
| LipidBB1 downstream pathway | 1/106 | 7.77E−01 | 8.35E−01 | 0.67 | BCL2L1 |
| Messenger RNA processing | 2/203 | 7.80E−01 | 8.37E−01 | 0.70 | SF3B1; SRPK1 |
| G alpha i pathway | 1/108 | 7.83E−01 | 8.39E−01 | 0.66 | GNAI1 |
| Platelet activation, signaling and | 2/205 | 7.84E−01 | 8.39E−01 | 0.69 | F2R; GNAI1 |
| aggregation | | | | | |
| Signaling by insulin receptor | 1/109 | 7.86E−01 | 8.39E−01 | 0.65 | TLR9 |
| Epidermal growth factor | 1/111 | 7.92E−01 | 8.44E−01 | 0.64 | NCK2 |
| receptor (EGFR) pathway | | | | | |
| Fas signaling pathway | 1/115 | 8.03E−01 | 8.55E−01 | 0.62 | NFATC3 |
| HIV life cycle | 1/118 | 8.11E−01 | 8.62E−01 | 0.60 | SSRP1 |
| MAPK signaling pathway | 3/314 | 8.19E−01 | 8.67E−01 | 0.68 | MAPKAPK5; MAPT; GADD45G |
| Lysosome | 1/121 | 8.19E−01 | 8.67E−01 | 0.59 | LAMP1 |
| p75 neurotrophin receptor- | 1/124 | 8.27E−01 | 8.74E−01 | 0.57 | BCL2L11 |
| mediated signaling | | | | | |
| G alpha (s) signaling events | 1/125 | 8.29E−01 | 8.75E−01 | 0.57 | GNAI1 |
| Spliceosome | 1/127 | 8.34E−01 | 8.78E−01 | 0.56 | SF3B1 |
| Signaling by FGFR in disease | 1/128 | 8.36E−01 | 8.78E−01 | 0.55 | CDKN1B |
| Wnt signaling pathway | 2/231 | 8.37E−01 | 8.78E−01 | 0.61 | F2R; NFATC3 |
| PDGFB signaling pathway | 1/129 | 8.39E−01 | 8.78E−01 | 0.55 | NCK2 |
| Parkinson's disease | 1/131 | 8.43E−01 | 8.82E−01 | 0.54 | UBA1 |
| Tight junction | 1/133 | 8.48E−01 | 8.83E−01 | 0.53 | GNAI1 |
| Membrane trafficking | 1/133 | 8.48E−01 | 8.83E−01 | 0.53 | CHMP4A |
| Signal transduction | 11/1020 | 8.51E−01 | 8.85E−01 | 0.76 | CDKN1B; BCL2L11; F2R; FFAR4; NCK2; DTX1; ITGA7; |
| | | | | | CCL2; AP2B1; CHRDL1; GNAI1 |
| Oxidative phosphorylation | 1/136 | 8.54E−01 | 8.86E−01 | 0.52 | COX17 |
| Adrenergic pathway | 1/137 | 8.56E−01 | 8.86E−01 | 0.52 | GNAI1 |
| Natural killer cell-mediated | 1/137 | 8.56E−01 | 8.86E−01 | 0.52 | NFATC3 |
| cytotoxicity | | | | | |
| Capped intron-containing pre- | 1/138 | 8.58E−01 | 8.86E−01 | 0.51 | SF3B1 |
| mRNA processing | | | | | |
| Biological oxidations | 1/139 | 8.60E−01 | 8.87E−01 | 0.51 | ALDH2 |
| SLC-mediated transmembrane | 2/251 | 8.69E−01 | 8.95E−01 | 0.56 | SLC35A1; SLC4A8 |
| transport | | | | | |
| Calcium regulation in the | 1/149 | 8.79E−01 | 9.03E−01 | 0.47 | GNAI1 |
| cardiac cell | | | | | |
| BDNF signaling pathway | 2/261 | 8.83E−01 | 9.06E−01 | 0.54 | ALDH6A1; BCL2L11 |
| Integrin signaling pathway | 1/155 | 8.89E−01 | 9.10E−01 | 0.46 | CAPNS1 |
| Neuroactive ligand-receptor | 2/272 | 8.97E−01 | 9.17E−01 | 0.52 | THRA; F2R |
| interaction | | | | | |
| Alzheimer's disease | 1/169 | 9.09E−01 | 9.27E−01 | 0.42 | MAPT |
| Metapathway | 1/174 | 9.15E−01 | 9.32E−01 | 0.40 | GLYATL2 |
| biotransformation | | | | | |
| Calcium signaling pathway | 1/178 | 9.20E−01 | 9.35E−01 | 0.40 | F2R |
| Transcription | 1/181 | 9.23E−01 | 9.37E−01 | 0.39 | SSRP1 |
| Huntington's disease | 1/184 | 9.26E−01 | 9.39E−01 | 0.38 | AP2B1 |
| GPCR ligand binding | 3/410 | 9.29E−01 | 9.40E−01 | 0.51 | F2R; FFAR4; CCL2 |
| G alpha (i) signaling events | 1/199 | 9.40E−01 | 9.50E−01 | 0.35 | GNAI1 |
| Gastrin-CREB signaling pathway | 1/206 | 9.46E−01 | 9.54E−01 | 0.34 | F2R |
| via PKC and MAPK | | | | | |
| Focal adhesion | 1/233 | 9.63E−01 | 9.70E−01 | 0.30 | ITGA7 |
| Class A GPCRs (rhodopsin-like) | 1/253 | 9.72E−01 | 9.77E−01 | 0.28 | F2R |
| Transmembrane transport of | 2/432 | 9.85E−01 | 9.88E−01 | 0.32 | SLC35A1; SLC4A8 |
| small molecules | | | | | |
| Hemostasis pathway | 2/468 | 9.90E−01 | 9.92E−01 | 0.30 | F2R; GNAI1 |
| Signaling by GPCR | 4/977 | 1.00E+00 | 1.00E+00 | 0.28 | F2R; FFAR4; CCL2; GNAI1 |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating subjects at risk for, or suffering from a metabolic disease comprising administering to a subject in need thereof, a therapeutically effective amount of one or more agents that:
increases the expression or activity of COBLL1, BCL2, or KDSR in one or more lipid-accumulating cells;
reduces the expression or activity of VPS4B in one or more lipid-accumulating cells;
enhances actin remodeling in one or more lipid-accumulating cells; or
inhibits apoptosis in one or more lipid-accumulating cells.

2. The method of claim 1, wherein the one or more lipid-accumulating cells is selected from the group consisting of adipocyte progenitors, adipocytes, and skeletal muscle.

3. The method of claim 1 or 2, wherein the metabolic disease is Type-2 Diabetes (T2D), MONW/MOH, lipodystrophy, insulin resistance with a “lipodystrophy-like” fat distribution, insulin sensitivity, BMI-adjusted T2D, and/or increased BMI-adjusted waist-to-hip ratio (WHRadjBMI).

4. The method of any of claims 1 to 3, wherein the subject has decreased expression of COBLL1 in adipocytes and/or adipocyte progenitors; decreased expression of BCL2 and/or KDSR in adipose-derived mesenchymal stem cells (AMSCs); decreased expression of BCL2 in skeletal muscle; and/or increased expression of VPS4B in AMSCs.

5. The method of any of claims 1 to 4, wherein the subject has an impairment of actin cytoskeleton remodeling in adipocytes and/or adipocyte progenitors; and/or comprises one or more MONW/MOH risk loci, preferably, the rs6712203 variant.

6. The method of any of claims 1 to 4, wherein the subject has decreased expression of BCL2 and/or KDSR in adipose-derived mesenchymal stem cells (AMSCs), decreased expression of BCL2 in skeletal muscle, increased expression of VPS4B in AMSCs, and/or increased apoptosis in adipocytes; and/or comprises one or more lipodystrophy risk loci, preferably, the rs12454712 variant.

7. The method of any of claims 1 to 5, wherein the one or more agents that enhances actin remodeling is selected from the group consisting of geodiamolides (Geodiamolide H), Jasplakinolide, Chondramide (Chondramide A), ADF/Cofilin, Arp2/3 complex, Profilin, Gelsolin (Flightless-I), Formin, Villin (Advillin), and Adseverin.

8. The method of claim 7, wherein the metabolic disease is Type-2 Diabetes (T2D) and/or MONW/MOH.

9. The method of any of claim 1 to 4 or 6, wherein the one or more agents that inhibits apoptosis is selected from the group consisting of Ginkgo biloba extract (EGb 761), Rhodiola crenulata extract (RCE), salidroside, dehydroepiandrosterone, allopregnanolone, diosmin, glycine, M50054, BI-6C9, TC9-305 (2-sulfonyl-pyrimidinyl derivatives), BI-11A7, 3-O-tolylthiazolidine-2,4-dione, minocycline, methazolamide, melatonin, gamma-tocotrienol (GTT), 3-hydroxypropyl-triphenylphosphonium (TPP)-conjugated imidazole-substituted oleic acid (TPP-IOA), TPP-conjugated stearic acid (TPP-ISA), TPP-6-ISA, CLZ-8, Xanthan gum (XG), PD98059, Vitamin E, and Tanshinone.

10. The method of claim 9, wherein the metabolic disease is lipodystrophy, insulin resistance with a “lipodystrophy-like” fat distribution, insulin sensitivity, BMI-adjusted T2D, increased BMI-adjusted waist-to-hip ratio (WHRadjBMI), and/or Type-2 Diabetes (T2D).

11. The method of any of claims 1 to 5, wherein the expression or activity of COBLL1 is increased in adipocyte progenitors or adipocytes.

12. The method of claim 11, wherein the metabolic disease is Type-2 Diabetes (T2D) and/or MONW/MOH.

13. The method of any of claim 1 to 4 or 6, wherein the expression or activity of BCL2 or KDSR is increased in adipocyte progenitors.

14. The method of claim 13, wherein the adipocyte progenitors are subcutaneous adipose-derived mesenchymal stem cells (AMSCs).

15. The method of any of claim 1 to 4 or 6, wherein the expression or activity of BCL2 is increased in skeletal muscle.

16. The method of any of claim 1 to 4 or 6, wherein the expression or activity of VPS4B is reduced in adipocyte progenitors.

17. The method of claim 16, wherein the adipocyte progenitors are visceral AMSCs.

18. The method of any of claims 13 to 17, wherein the metabolic disease is lipodystrophy, insulin resistance with a “lipodystrophy-like” fat distribution, insulin sensitivity, BMI-adjusted T2D, increased BMI-adjusted waist-to-hip ratio (WHRadjBMI), and/or Type-2 Diabetes (T2D).

19. The method of any of claims 1 to 5, wherein the one or more agents are one or more small molecules that enhances the activity or expression of COBLL1.

20. The method of any of claim 1 to 4 or 6, wherein the one or more agents are one or more small molecules that enhances the activity or expression of BCL2 or KDSR.

21. The method of 1 to 4 or 6, wherein the one or more agents are one or more small molecules that reduces the activity or expression of VPS4B.

22. The method of any of claims 1 to 5, where the one or more agents is a polynucleotide comprising a sequence encoding COBLL1.

23. The method of claim 22, wherein the polynucleotide is part of a vector system comprising adipocyte specific regulatory sequences for tissue specific expression of the one or more agents.

24. The method of claim 23, wherein the vector system comprises a viral vector system.

25. The method of claim 24, wherein the viral vector system has tropism for adipose tissue.

26. The method of any of claims 1 to 5, wherein the one or more agents is a recombinant polypeptide derived from the COBLL1 gene or functional variant thereof.

27. The method of any of claims 1 to 6, wherein the one or more agents is a fusion protein, comprising a DNA binding element of a programmable nuclease configured to specifically bind to a sequence in proximity to the COBLL1 gene and wherein the protein activates expression of COBLL1; or configured to specifically bind to a sequence in proximity to the 18q21.33 locus and wherein the protein activates expression of BCL2 and/or KDSR.

28. The method of claim 27, wherein the DNA-binding portion comprises a zinc finger protein or DNA-binding domain thereof, TALE protein or DNA-binding domain thereof, or a Cas nuclease protein or DNA-binding domain thereof.

29. The method of any of claims 27 to 28, wherein the DNA-binding portion is linked to an activation domain.

30. The method of claim 29, wherein the activation domain is derived from an alternative splicing variant of POU2F2 that activates expression.

31. The method of any one of claims 27 to 30, wherein the fusion protein is encoded in a polynucleotide vector.

32. The method of claim 31, wherein the vector system comprises adipocyte specific regulatory sequences for tissue specific expression of the one or more agents.

33. The method of claim 31, wherein the vector system comprises a viral vector system optionally comprising a tropism for adipose tissue.

34. A method of treating subjects suffering from or at risk of developing Type-2 Diabetes or lipodystrophy, comprising administering a gene editing system that corrects one or more genomic variants that decrease the expression or activity of COBLL1 in adipocytes and/or adipocyte progenitors; or that decrease the expression or activity of BCL2 and KDSR in adipocyte progenitors, decrease the expression or activity of BCL2 in skeletal muscle, and increase the expression or activity of VPS4B in adipocyte progenitors.

35. A method of treating subjects suffering from or at risk of developing a metabolic disease, comprising administering a gene editing system that corrects one or more genomic risk variants selected from the group consisting of rs6712203, rs9686661, rs4804833, rs2972144, rs13389219, rs11837287, rs7903146, rs1534696, rs287621, rs1412956, rs13133548, rs11667352, rs12454712 (BCL2), rs673918, rs646123, rs2963449, rs1572993, rs632057, rs11637681, rs6063048, rs7660000, rs1421085, rs7258937, rs9939609, rs998584, rs4925109, rs12641088, and any variant that is within the haplotype for the above variants.

36. The method of claim 34 or 35, wherein the gene editing system is a zinc finger nuclease, a TALEN, a meganuclease, or a CRISPR-Cas system.

37. The method of claim 36, wherein the gene editing system is a CRISPR-Cas system.

38. The method of claim 37, further comprising a donor template, configured to replace a portion of a genomic sequence comprising the one or more genomic risk variants with a wild-type or non-risk variant.

39. The method of claim 34 or 35, wherein the one or more variants comprises rs6712203 or rs12454712.

40. The method of claim 34 or 35, wherein the gene editing system is a base editing system that corrects one or more of the genomic variants to a wild type or non-risk variant.

41. The method of claim 40, wherein the base editing system is a CRISPR-Cas base editing system.

42. The method of claim 40, wherein the one or more genomic variants include rs6712203 or rs12454712.

43. The method of claim 42, wherein a C allele/risk genotype of rs6712203 is edited to the T allele/non-risk genotype; or wherein a T allele/risk genotype of rs12454712 is edited to the C allele/non-risk genotype.

44. The method claim 34 or 35, wherein the gene editing system is a prime editing system that corrects one or more of the genomic variants to a wild type or non-risk variant.

45. The method of claim 44, wherein the one or more genomic variants include rs6712203 or rs12454712.

46. The method of claim 45, wherein the PEG RNA encodes a donor template to replace the rs6712203 or rs12454712 variant with a wild-type or non-risk variant.

47. The method claim 34 or 35, wherein the gene editing system is a programmable transposition system that corrects one or more of the genomic variants to a wild type or non-risk variant.

48. The method of claim 47, wherein the one or more genomic variants include rs6712203 or rs12454712.

49. The method of claim 47 or 48, wherein the programmable transposition system is a CAST system.

50. The method of claim 49, wherein the guide polynucleotide of the CAST system comprises a donor construct comprising a donor sequence to replace a genomic region comprising the rs6712203 or rs12454712 variant with a wild type sequence.

51. A method of treating Type-2 Diabetes in subjects comprising one or more variants that decrease COBLL1 expression or activity by decreasing binding of POU2F2 to a binding site in an enhancer regulating COBLL1 expression comprising, administering to a subject in need thereof 1) allogenic adipocyte progenitors that exhibit wild type COBLL1 expression, or 2) autologous adipocyte progenitors genetically edited to correct the one or more variants to a wild-type sequence.

52. A method of treating a metabolic disorder in subjects comprising administering to a subject in need thereof
1) allogenic adipocyte progenitors that do not comprise one or more genomic risk variants selected from the group consisting of rs6712203, rs9686661, rs4804833, rs2972144, rs13389219, rs11837287, rs7903146, rs1534696, rs287621, rs1412956, rs13133548, rs11667352, rs12454712 (BCL2), rs673918, rs646123, rs2963449, rs1572993, rs632057, rs11637681, rs6063048, rs7660000, rs1421085, rs7258937, rs9939609, rs998584, rs4925109, rs12641088, and any variant that is within the haplotype for the above variants; or,
2) autologous adipocyte progenitors genetically edited to correct the one or genomic risk variants to a wild-type or non-risk variant.

53. The method of claim 51 or 52, wherein the one or more variants comprise rs6712203 or rs12454712.

54. The method of any of claims 51 to 53, wherein the adipocyte progenitors are adipose-derived mesenchymal stem cells (AMSCs).

55. The method of claim 51, wherein the autologous adipocyte progenitors are edited to change a C allele/risk genotype of rs6712203 to the T allele/non-risk genotype.

56. A method for detecting a variant in subject, comprising, detecting whether a rs6712203 or rs12454712 variant is present in a subject by conducting a genotyping assay on a biological sample from the subject and detecting whether the rs6712203 or rs12454712 variant is present.

57. The method of claim 56, wherein genotyping is conducted by restriction fragment length polymorphism identification, random amplified polymorphic detection, amplified fragment length polymorphism, PCR, DNA sequencing, allele specific oligonucleotide hybridization, or microarray hybridization.

58. The method of claim 56, further comprising administering a) a therapeutically effective amount of one or more agents that increase the expression or activity of COBLL1, or enhance actin remodeling in adipocytes or adipocyte progenitors, b) a therapeutically effective amount of one or more agents that increase the expression or activity of BCL2 and/or KDSR, or inhibit apoptosis in adipocytes or adipocyte progenitors, c) a gene editing system that corrects the one or more variants to a wild type sequence, d) adoptive cell transfer comprising allogenic adipocyte or adipocyte progenitor donors exhibiting wild type COBLL1 expression, or autologous adipocyte or adipocyte progenitor donors genetically modified to correct the one or more variants to a wild type sequence, or e) adoptive cell transfer comprising allogenic adipocyte progenitor donors exhibiting wild type BCL2 and/or KDSR expression, or autologous adipocyte progenitor donors genetically modified to correct the one or more variants to a wild type sequence.

59. A method of treating T2D comprising:
performing a genotyping assay on a biological sample from a subject to determine if the subject has one or more variants that decrease COBLL1 expression or activity by decreasing binding of POU2F2 to a binding site in an enhancer regulating COBLL1 expression; and
if the subject has the one or more variants administering a) a therapeutically effective amount of one or more agents that increase the expression or activity of COBLL1, or enhance actin remodeling in adipocytes or adipocyte progenitors, b) a gene editing system that corrects the one or more variants to a wild type sequence, or c) adoptive cell transfer comprising allogenic adipocyte donors exhibiting wild type COBLL1 expression, or autologous adipocyte donors genetically modified to correct the one or more variants to a wild type sequence; or
if the subject does not have the one or more variants, administering a standard-of-care T2D therapy.

60. A method of treating lipodystrophy comprising:
performing a genotyping assay on a biological sample from a subject to determine if the subject has one or more variants that decrease the expression or activity of BCL2 and KDSR in adipocyte progenitors, decrease the expression or activity of BCL2 in skeletal muscle, and increase the expression or activity of VPS4B in adipocyte progenitors; and
if the subject has the one or more variants administering a) a therapeutically effective amount of one or more agents that increase the expression or activity of BCL2 and/or KDSR, or inhibit apoptosis in adipocytes or adipocyte progenitors, b) a gene editing system that corrects the one or more variants to a wild type sequence, or c) adoptive cell transfer comprising allogenic adipocyte progenitor donors exhibiting wild type BCL2 and/or KDSR expression, or autologous adipocyte progenitor donors genetically modified to correct the one or more variants to a wild type sequence; or
if the subject does not have the one or more variants, administering a standard-of-care lipodystrophy therapy.

61. A method for diagnosing metabolically obese normal weight (MONW) subjects at increased risk for developing T2D comprising, detecting one or more variants that decrease the expression or activity of COBLL1 in adipocyte and/or adipocyte progenitors and diagnosing the subject as increased risk of T2D if the one or more variants are detected.

62. The method of claim 61, wherein the one or more variants decrease binding of POU2F2 to a binding site in an enhancer regulating COBLL1 expression.

63. The method of claim 62, wherein the one or more variants comprises rs6712203.

64. A method for diagnosing lipodystrophy subjects at increased risk for developing T2D or heart disease comprising, detecting one or more variants that that decrease the expression or activity of BCL2 and KDSR in adipocyte progenitors, decrease the expression or activity of BCL2 in skeletal muscle, and increase the expression or activity of VPS4B in adipocyte progenitors and diagnosing the subject as increased risk of T2D or heart disease if the one or more variants are detected.

65. The method of claim 64, wherein the one or more variants comprises rs12454712.

66. A method of screening for agents capable of treating T2D in subjects with a MONW/MOH risk phenotype comprising:
a) treating a population of cells comprising adipocytes having the rs6712203 variant with an agent; and
b) detecting actin remodeling and/or one or more COBLL1 co-regulated genes,
wherein detecting an increase in actin remodeling and/or the one or more genes identifies agent as capable of treating T2D in subjects having a MONW/MOH risk phenotype.

67. The method of claim 66, wherein the one or more COBLL1 co-regulated genes are selected from the group consisting of ITGAM, PIK3CA, ROCK2, ITGA1, ARHGEF7, CRK, FGFR2, and ARHGEF6.

68. A method of screening for agents capable of treating lipodystrophy in subjects with a lipodystrophy risk phenotype comprising:
a) treating a population of cells comprising adipocytes having the rs12454712 variant with an agent; and
b) detecting apoptosis and/or one or more apoptosis genes,
wherein detecting a decrease in apoptosis and/or one or more apoptosis genes identifies agent as capable of treating lipodystrophy in subjects having a lipodystrophy risk phenotype.

69. An unbiased high-throughput multiplex profiling method for simultaneously identifying morphological and cellular phenotypes for lipid-accumulating cells comprising:
a. staining a cellular system comprising one or more lipid-accumulating cells with one or more stains that differentiate cellular compartments selected from the group consisting of nuclei, cytoplasm and total cell and differentiate organelles selected from the group consisting of DNA, mitochondria, actin, Golgi, plasma membrane, lipids, nucleoli and cytoplasmic RNA;
b. imaging the stained cells using an automated image analysis pipeline; and
c. identifying one or more morphological features for each of the organelles from the resulting images, wherein the features comprise one or more features selected from the group consisting of object size, object shape, intensity, granularity, texture, colocalization, number of objects, distance to neighboring objects, cellular compartment, and combinations thereof.

70. The method of claim 69, wherein about 100 or more cells are imaged for the cellular system.

71. The method of claim 69, wherein about 500 or more cells are imaged for the cellular system.

72. The method of any of claims 69 to 71, wherein each feature for each organelle includes a quantitative range comprising at least two values for the feature.

73. The method of any of claims 69 to 72, wherein a pattern of morphological features is linked to a cellular phenotype.

74. The method of any of claims 69 to 73, wherein the morphological features are linked to one or more gene expression programs.

75. The method of any of claims 69 to 74, wherein the cellular system is obtained from a subject.

76. The method of any of claims 69 to 75, wherein the cellular system comprises lipocytes.

77. The method of claim 76, wherein the lipocytes are selected from the group consisting of adipocytes, hepatocytes, macrophages/foam cells and glial cells.

78. The method of claim 76, wherein the lipocytes are part of a pathophysiological process in cells selected from the group consisting of vascular smooth muscle cells, skeletal muscle cells, renal podocytes, and cancer cells.

79. The method of any of claims 69 to 78, wherein the cellular system comprises stem cells differentiated over a time course, wherein the cells from the cellular system are stained and imaged at different time points.

80. The method of claim 79, wherein the time points comprise one or more time points selected from the group consisting of 0 days, 3 days, 8 days and 14 days.

81. The method of any of claims 69 to 80, wherein the cellular system comprises adipose-derived mesenchymal stem cells (AMSCs) differentiated to adipocytes, wherein the cellular system is stained over a time course.

82. The method of claim 81, wherein the AMSCs are obtained from a subject.

83. The method of claim 81 or 82, wherein the AMSCs are subcutaneous AMSCs.

84. The method of claim 81 or 82, wherein the AMSCs are visceral AMSCs.

85. The method of any of claims 69 to 84, further comprising performing RNA-seq on the lipid-accumulating cells.

86. The method of any of claims 69 to 85, wherein the cellular system is stained with one or more fluorescent dyes selected from the group consisting of Hoechst, MitoTracker Red, Phalloidin, wheat germ agglutinin (WGA), BODIPY, and SYTO14.

87. The method of claim 86, wherein the imaging is taken across four channels.

88. The method of any of claims 69 to 87, wherein the image analysis pipeline comprises image analysis software and a novel algorithm.

89. The method of any of claims 68 to 88, wherein cells are clustered based on patterns of features identified.

90. The method of any of claims 69 to 89, wherein the imaging pipeline comprises artificial intelligence, machine learning, deep learning, neural networks, and/or linear regression modeling.

91. The method of any of claims 69 to 90, wherein the cellular system comprises cells comprising a SNP of interest, whereby morphological and cellular phenotypes can be determined for the SNP.

92. The method of any of claims 69 to 90, wherein the cellular system comprises cells perturbed with one or more drugs, whereby morphological and cellular phenotypes can be determined for the one or more drugs.

93. The method of any of claims 69 to 90, wherein the cellular system comprises cells perturbed at one or more genomic loci, whereby morphological and cellular phenotypes can be determined for the one or more genomic loci.

94. The method of claim 93, wherein the cells are perturbed with a programmable nuclease or RNAi.

95. A method of identifying morphological and cellular features for predicting metabolic clinical characteristics in a subject in need thereof comprising:
a. identifying morphological and cellular features according to the method of any of claims 69 to 94 for one or more cellular systems derived from one or more subjects having a metabolic clinical characteristic; and
b. fitting a logistic regression model for the clinical characteristic on the entire set of features from (a) and selecting features that best fit the model.

96. The method of claim 95, further comprising:
b′. identifying a subset of features from (a) comprising:
i. constructing an interaction network between the features, wherein nodes represent features, edges indicate interactions between two nodes, and edge weight indicates the strength of the interaction, and
ii. selecting a subset of nodes with at least one edge above a cutoff weight, whereby features with high-weight interactions are selected; and
c′. fitting a logistic regression model for the clinical characteristic on the entire set of features from (b′) and selecting features that best fit the model.

97. The method of claim 95 or 96, further comprising grouping the features into a compartment category selected from the group consisting of lipid, actin/Golgi/plasma membrane (AGP), Mito, DNA, and other, and stratifying by differentiation day, wherein the number of features that can be modeled in every grouped and stratified category are the features.

98. A method of predicting metabolic clinical characteristics in a subject in need thereof comprising:
a. identifying morphological and cellular features according to the method of any of claims 69 to 94 for one or more cellular systems derived from the subject; and
b. estimating a metabolic clinical characteristic from one or more of the features.

99. The method of claim 98, wherein the one or more features used for estimating the clinical characteristic are selected according to claims 95 to 97.

100. A method of identifying histological features for predicting metabolic clinical characteristics in a subject in need thereof comprising:
a. identifying features for one or more histological images of adipose tissue samples obtained from one or more subjects having a metabolic clinical characteristic, wherein the features are identified by a method comprising:
i. grouping at least 100-500 cells from an image into cell area (μm²) categories, wherein the categories are defined by cell area ranges for a plurality of control subjects of the same sample tissue type;
ii. determining for each cell area category one or more features selected from: the fraction of cells in the cell area category, median area of cells in the category, 25% interquartile point in the category, and 75% interquartile point in the category; and
b. fitting a logistic regression model for the clinical characteristic on the entire set of features and selecting features that best fit the model.

101. The method of claim 100, wherein the cells are grouped into 5 area categories consisting of:
i. a cell area <25% quartile point for the control group (very small),
ii. a cell area ≥25% quartile point for the control group and <the median cell area for the control group (small),
iii. a cell area ≥median cell area for the control group and <mean cell area for the control group (medium),
iv. a cell area ≥mean area for the control group and <75% quartile point for the control group (large), and
v. a cell area ≥75% quartile point for the control group (very large).

102. A method of predicting metabolic clinical characteristics in a subject in need thereof comprising:
a. identifying features from a histological image of an adipose tissue sample obtained from the subject comprising:
i. grouping at least 100-500 cells from the image into cell area (μm²) categories, wherein the categories are defined by cell area ranges for a plurality of control subjects of the same cell tissue type;
ii. determining for each cell area category one or more features selected from the fraction of cells in the cell area category, median area of cells in the category, 25% interquartile point in the category, and 75% interquartile point in the category; and
b. estimating a metabolic clinical characteristic from one or more of the features.

103. The method of claim 102, wherein the cells are grouped into 5 area categories consisting of:
i. a cell area <25% quartile point for the control group (very small),
ii. a cell area ≥25% quartile point for the control group and <the median cell area for the control group (small),
iii. a cell area ≥median cell area for the control group and <mean cell area for the control group (medium),
iv. a cell area ≥mean area for the control group and <75% quartile point for the control group (large), and
v. a cell area ≥75% quartile point for the control group (very large).

104. The method of claim 102 or 103, wherein the one or more features used for estimating the clinical characteristic are selected according to claim 100 or 101.

105. The method of any of claims 100 to 104, wherein the tissue is subcutaneous adipose tissue.

106. The method of any of claims 100 to 104, wherein the tissue is visceral adipose tissue.

107. A method of predicting metabolic clinical characteristics in a subject in need thereof comprising determining clinical characteristics according to claims 98 to 99 and according to claims 102 to 106; and comparing the clinical characteristics to predict clinical characteristics for the subject.

108. The method of any of claims 95 to 107, wherein the logistic regression model is a linear model with logit link (GLM).

109. The method of claim 108, wherein the linear association with binomial distribution is implemented using the R glm function,
wherein the default glm convergence criteria on deviances is used to stop the iterations,
wherein the DeLong method is used to calculate confidence intervals for the c-statistics,
wherein forward feature selection (R step function) is used to select the features, and/or
wherein the Akaike information criterion (AIC) is used as the stop condition for the feature selection procedure.

110. A method of detecting HOMO-IR or WHRadjBMI risk in a subject comprising, detecting one or more features according to the method of any of claims 69 to 94, wherein the one or more features are selected from the group consisting of:
a. increased lipid granularity in visceral adipocytes;
b. increased lipid texture_SumEntropy in visceral adipocytes;
c. increased cell area/shape in visceral adipocytes;
d. decreased lipid texture_InverseDifferenceMoment in visceral adipocytes;
e. decreased BODIPY Texture_AngularSecondMoment;
f. upregulation of one or more genes selected from the group consisting of GYS-1, TPI1, PFKP and PGK; and
g. downregulation of one or more genes selected from the group consisting of ACAA1 and SCP2.

111. A method of detecting lipodystrophy risk in a subject comprising, detecting one or more features according to the method of any of claims 69 to 94, wherein the one or more features are selected from the group consisting of:
a. increased mitochondrial stain intensity;
b. smaller lipid droplets on average compared to adipocytes from individuals with low polygenic risk;
c. upregulation of one or more genes selected from the group consisting of EHHADH and NFATC3.

112. The method of claim 110 or 111, further comprising a treatment step comprising administering one or more of insulin, thiazolidinedione, biguanide, meglitinide, DPP-4 inhibitors, Sodium-glucose transporter 2 (SGLT2) inhibitor, alpha-glucosidase inhibitor, bile acid sequestrant, sulfonylureas and/or amylin analogs.


SEQUENCE LISTING (84 sequences)

SEQ ID NO: 1 - protein, 1128 aa, Homo sapiens
MDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 2 - protein, 1166 aa, Homo sapiens
MDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPETFHPGLSSQEQCTAPKLMEETSVFECPGTPEAAITSLTSGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 3 - protein, 1233 aa, Homo sapiens
MPPSWSPLMCGRAAEAAASSRTPGREMGQAVTRRLGAGARAAPRRAMDGRTPRPQDAPARREIAGSWRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRATSVTVFSKSSLQESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPETFHPGLSSQEQCTAPKLMEETSVFECPGTPEAAITSLTSGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 4 - protein, 1174 aa, Homo sapiens
MPPSWSPLMCGRAAEAAASSRTPGREMGQAVTRRLGAGARAAPRRAMDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 5 - protein, 1128 aa, Homo sapiens; identical to SEQ ID NO: 1

SEQ ID NO: 6 - protein, 1173 aa, Homo sapiens
MPPSWSPLMCGRAAEAAASSRTPGREMGQAVTRRLGAGARAAPRRAMDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 7 - protein, 1161 aa, Homo sapiens
MPPSWSPLMCGRAAEAAASSRTPGREMGQAVTRRLGAGARAAPRRAMDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRATSVTVFSKSSLQESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRDG

SEQ ID NO: 8 - protein, 1128 aa, Homo sapiens; identical to SEQ ID NO: 1

SEQ ID NO: 9 - protein, 1141 aa, Homo sapiens
MDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRATSVTVFSKSSLQESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDGH

SEQ ID NO: 10 - protein, 1140 aa, Homo sapiens
MDGRTPRPQDAPARRKPKAKAPLPPAETKYTDVSSAADSVESTAFIMEQKENMIDKDVELSVVLPGDIIKSTTVHGSKPMMDLLIFLCAQYHLNPSSYTIDLLSAEQNHIKFKPNTPIGMLEVEKVILKPKMLDKKKPTPIIPEKTVRVVINFKKTQKTIVRVSPHASLQELAPIICSKCEFDPLHTLLLKDYQSQEPLDLTKSLNDLGLRELYAMDVNRATSVTVFSKSSLQESCQISQNLDIMKEKENKGFFSFFQRSKKKRDQTASAPATPLVNKHRPTFTRSNTISKPYISNTLPSDAPKKRRAPLPPMPASQSVPQDLAHIQERPASCIVKSMSVDETDKSPCEAGRVRAGSLQLSSMSAGNSSLRRTKRKAPSPPSKIPPHQSDENSRVTALQPVDGVPPDSASEANSPEELSSPAGISSDYSLEEIDEKEELSEVPKVEAENISPKSQDIPFVSTDIINTLKNDPDSALGNGSGEFSQNSMEEKQETKSTDGQEPHSVVYDTSNGKKVVDSIRNLKSLGPNQENVVQNEIIVYPENTEDNMKNGVKKTEINVEGVAKNNNIDMEVERPSNSEAHETDTAISYKENHLAASSVPDQKLNQPSAEKTKDAAIQTTPSCNSFDGKHQDHNLSDSKVEECVQTSNNNISTQHSCLSSQDSVNTSREFRSQGTLIIHSEDPLTVKDPICAHGNDDLLPPVDRIDKNSTASYLKNYPLYRQDYNPKPKPSNEITREYIPKIGMTTYKIVPPKSLEISKDWQSETIEYKDDQDMHALGKKHTHENVKETAIQTEDSAISESPEEPLPNLKPKPNLRTEHQVPSSVSSPDDAMVSPLKPAPKMTRDTGTAPFAPNLEEINNILESKFKSRASNAQAKPSSFFLQMQKRVSGHYVTSAAAKSVHAAPNPAPKELTNKEAERDMLPSPEQTLSPLSKMPHSVPQPLVEKTDDDVIGQAPAEASPPPIAPKPVTIPASQVSTQNLKTLKTFGAPRPYSSSGPSPFALAVVKRSQSFSKERTESPSASALVQPPANTEEGKTHSVNKFVDIPQLGVSDKENNSAHNEQNSQIPTPTDGPSFTVMRQSSLTFQSSDPEQMRQSLLTAIRSGEAAAKLKRVTIPSNTISVNGRSRLSHSMSPDAQDG

SEQ ID NO: 11 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 12 - protein, 205 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 13 - protein, 233 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNRWFLTGMTVAGVVLLGSLFSRK

SEQ ID NO: 14 - protein, 170 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQDTFVELYGNNAAAESRKGQERFNRWFLTGMTVAGVVLLGSLFSRK

SEQ ID NO: 15 - protein, 227 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWVRTKPLVCPFSLASGQRSPTALLLYLFLLCWVIVGDVDS

SEQ ID NO: 16 - protein, 170 aa, Homo sapiens; identical to SEQ ID NO: 14

SEQ ID NO: 17 - protein, 327 aa, Homo sapiens
MCSTSGCDLEEIPLDDDDLNTIEFKILAYYTRHHVFKSTPALFSPKLLRTRSLSQRGLGNCSANESWTEVSWPCRNSQSSEKAINLGKKKSSWKAFFGVVEKEDSQSTPAKVSAQGQRTLEYQDSHSQQWSRCLSNVEQCLEHEAVDPKVISIANRVAEIVYSWPPPQATQAGGFKSKEIFVTEGLSFQLQGHVPVASSSKKDEEEQILAKIVELLKYSGDQLERKLKKDKALMGHFQDGLSYSVFKTITDQVLMGVDPRGESEVKAQGFKAALVIDVTAKLTAIDNHPMNRVLGFGTKYLKENFSPWIQQHGGWEKILGISHEEVD

SEQ ID NO: 18 - protein, 204 aa, Homo sapiens
MVDQLRERTTMADPLRERTELLLADYLGYCAREPGTPEPAPSTPEAAVLRSAAARLRQIHRSFFSAYLGYPGNRFELVALMADSVLSDSPGPTWGRVVTLVTFAGTLLERGPLVTARWKKWGFQPRLKEQEGDVARDCQRLVALLSSRLMGQHRAWLQAQGGWDGFCHFFRTPFPLAFWRKQLVQAFLSCLLTTAFIYLWTRLL

SEQ ID NO: 19 - protein, 243 aa, Homo sapiens
MGRPAGLFPPLCPFLGFRPEACWERHMQIERAPSVPPFLRWAGYRPGPVRRRGKVELIKFVRVQWRRPQVEWRRRRWGPGPGASMAGSEELGLREDTLRVLAAFLRRGEAAGSPVPTPPRSPAQEEPTDFLSRLRRCLPCSLGRGAAPSESPRPCSLPIRPCYGLEPGPATPDFYALVAQRLEQLVQEQLKSPPSPELQGPPSTEKEAILRRLVALLEEEAEVINQKEGILAVSPVDLNLPLD

SEQ ID NO: 20 - protein, 332 aa, Homo sapiens
MLLLAAAFLVAFVLLLYMVSPLISPKPLALPGAHVVVTGGSSGIGKCIAIECYKQGAFITLVARNEDKLLQAKKEIEMHSINDKQVVLCISVDVSQDYNQVENVIKQAQEKLGPVDMLVNCAGMAVSGKFEDLEVSTFERLMSINYLGSVYPSRAVITTMKERRVGRIVFVSSQAGQLGLFGFTAYSASKFAIRGLAEALQMEVKPYNVYITVAYPPDTDTPGFAEENRTKPLETRLISETTSVCKPEQVAKQIVKDAIQGNFNSSLGSDGYMLSALTCGMAPVTSITEGLQQVVTMGLFRTIALFYLGSFDSIVRRCMMQREKSENADKTA

SEQ ID NO: 21 - protein, 8 aa, Homo sapiens
TGXXXGXG

SEQ ID NO: 22 - protein, 444 aa, Homo sapiens
MSSTSPNLQKAIDLASKAAQEDKAGNYEEALQLYQHAVQYFLHVVKYEAQGDKAKQSIRAKCTEYLDRAEKLKEYLKNKEKKAQKPVKEGQPSPADEKGNDSDGEGESDDPEKKKLQNQLQGAIVIERPNVKWSDVAGLEGAKEALKEAVILPIKFPHLFTGKRTPWRGILLFGPPGTGKSYLAKAVATEANNSTFFSISSSDLVSKWLGESEKLVKNLFQLARENKPSIIFIDEIDSLCGSRSENESEAARRIKTEFLVQMQGVGVDNDGILVLGATNIPWVLDSAIRRRFEKRIYIPLPEPHARAAMFKLHLGTTQNSLTEADFRELGRKTDGYSGADISIIVRDALMQPVRKVQSATHFKKVRGPSRADPNHLVDDLLTPCSPGDPGAIEMTWMDVPGDKLLEPVVSMSDMLRSLSNTKPTVNEHDLLKLKKFTEDFGQEG

SEQ ID NO: 23 - protein, 7 aa, Simian virus 40
PKKKRKV

SEQ ID NO: 24 - protein, 10 aa, Simian virus 40
PKKKRKVEAS

SEQ ID NO: 25 - protein, 16 aa, synthetic construct
KRPAATKKAGQAKKKK

SEQ ID NO: 26 - protein, 9 aa, Homo sapiens
PAAKRVKLD

SEQ ID NO: 27 - protein, 11 aa, Homo sapiens
RQRRNELKRSP

SEQ ID NO: 28 - protein, 38 aa, Homo sapiens
NQSSNFGPMKGGNFGGRSSGPYGGGGQYFAKPRNQGGY

SEQ ID NO: 29 - protein, 42 aa, Homo sapiens
RMRIZFKNKGKDTAELRRRRVEVSVELRKAKKDEQILKRRNV

SEQ ID NO: 30 - protein, 8 aa, Homo sapiens
VSRKRPRP

SEQ ID NO: 31 - protein, 8 aa, Homo sapiens
PPKKARED

SEQ ID NO: 32 - protein, 8 aa, Homo sapiens
PQPKKKPL

SEQ ID NO: 33 - protein, 12 aa, Mus sp.
SALIKKKKKMAP

SEQ ID NO: 34 - protein, 5 aa, Influenza virus
DRLRR

SEQ ID NO: 35 - protein, 7 aa, Influenza virus
PKQKKRK

SEQ ID NO: 36 - protein, 10 aa, Hepatitis D virus
RKLKKKIKKL

SEQ ID NO: 37 - protein, 10 aa, Mus sp.
REKKKFLKRR

SEQ ID NO: 38 - protein, 20 aa, Homo sapiens
KRKGDEVDGVDEVAKKKSKK

SEQ ID NO: 39 - protein, 17 aa, Homo sapiens
RKCLQAGMNLEARKTKK

SEQ ID NO: 40 - protein, 288 aa, synthetic construct
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 41 - protein, 183 aa, synthetic construct
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 42 - DNA, 20 bp, synthetic construct
  source 1..20: mol_type = other DNA
atacacaggtgcaatcaaag

SEQ ID NO: 43 - DNA, 20 bp, synthetic construct
  source 1..20: mol_type = other DNA
caactttgggatcagctccg

SEQ ID NO: 44 - DNA, 20 bp, synthetic construct
  source 1..20: mol_type = other DNA
tattgaacgcaccttaagtg

SEQ ID NO: 45 - DNA, 20 bp, synthetic construct
  source 1..20: mol_type = other DNA
agtcctcactggtggacacg

SEQ ID NO: 46 - DNA, 20 bp, synthetic construct
  source 1..20: mol_type = other DNA
caccaggtcatctctcaaga

SEQ ID NO: 47 - DNA, 20 bp, synthetic construct
  source 1..20: mol_type = other DNA
ttatatggccaatcccacta

SEQ ID NO: 48 - DNA, 20 bp, synthetic construct
  source 1..20: mol_type = other DNA
tcacggcagatacttaccag

SEQ ID NO: 49 - DNA, 20 bp, synthetic construct
  source 1..20: mol_type = other DNA
tctgcacggtgtatcgagag

SEQ ID NO: 50 - DNA, 20 bp, synthetic construct
  source 1..20: mol_type = other DNA
atcaggccttgtccgtgatt

SEQ ID NO: 51 - DNA, 20 bp, synthetic construct
  source 1..20: mol_type = other DNA
tacgtcattaagagttcaac

SEQ ID NO: 52 - DNA, 20 bp, synthetic construct
  source 1..20: mol_type = other DNA
gacagtgaaattagctccca

SEQ ID NO: 53 - DNA, 20 bp, synthetic construct
  source 1..20: mol_type = other DNA
gattcatactaaacactcta

SEQ ID NO: 54 - DNA, 20 bp, synthetic construct
  source 1..20: mol_type = other DNA
cctagttcataagctacgcc

SEQ ID NO: 55 - DNA, 34 bp, synthetic construct
  source 1..34: mol_type = other DNA
ctgtctcttatacacatctccgagcccacgagac

SEQ ID NO: 56 - DNA, 33 bp, synthetic construct
  source 1..33: mol_type = other DNA
ctgtctcttatacacatctgacgctgccgacga

SEQ ID NO: 57 - DNA, 40 bp, synthetic construct
  source 1..40: mol_type = other DNA
ttaatttgcctcattcatcacatgcaattctggcaaggaa

SEQ ID NO: 58 - DNA, 40 bp, synthetic construct
  source 1..40: mol_type = other DNA
ttaatttgcctcattcatcatatgcaattctggcaaggaa

SEQ ID NO: 59 - DNA, 40 bp, synthetic construct
  source 1..40: mol_type = other DNA
ccccacttccctctagggaatgggaaagaacatttaacct

SEQ ID NO: 60 - DNA, 40 bp, synthetic construct
  source 1..40: mol_type = other DNA
ccccacttccctctagggaacgggaaagaacatttaacct

SEQ ID NO: 61 - DNA, 25 bp, synthetic construct
  source 1..25: mol_type = other DNA
caccgactctccactaccattgcca

SEQ ID NO: 62 - DNA, 25 bp, synthetic construct
  source 1..25: mol_type = other DNA
aaactggcaatggtagtggagagtc

SEQ ID NO: 63 - DNA, 30 bp, synthetic construct
  source 1..30: mol_type = other DNA
ggtggtcccattaaaaagaaagaagcttgg

SEQ ID NO: 64 - DNA, 30 bp, synthetic construct
  source 1..30: mol_type = other DNA
cttctcttttaccctgctggctactggttg

SEQ ID NO: 65 - DNA, 23 bp, synthetic construct
  source 1..23: mol_type = other DNA
tcattcatcatatgcaattctgg

SEQ ID NO: 66 - DNA, 26 bp, synthetic construct
  source 1..26: mol_type = other DNA
ggcaaattaatatttaggattatatc

SEQ ID NO: 67 - DNA, 22 bp, synthetic construct
  source 1..22: mol_type = other DNA
ccattgccaacggctgagtcag

SEQ ID NO: 68 - DNA, 22 bp, synthetic construct
  source 1..22: mol_type = other DNA
tagtggagagttctcacaaaac

SEQ ID NO: 69 - DNA, 21 bp, synthetic construct
  source 1..21: mol_type = other DNA
tgggaaggaaaatgcattggg

SEQ ID NO: 70 - DNA, 20 bp, synthetic construct
  source 1..20: mol_type = other DNA
ataaggtcaggatggggtgg

SEQ ID NO: 71 - DNA, 20 bp, synthetic construct
  source 1..20: mol_type = other DNA
ctgtgccatcctgatgactg

SEQ ID NO: 72 - DNA, 18 bp, synthetic construct
  source 1..18: mol_type = other DNA
ccagggccaatctcaaaa

SEQ ID NO: 73 - DNA, 19 bp, synthetic construct
  source 1..19: mol_type = other DNA
tgaaaaggaccccacgaag

SEQ ID NO: 74 - DNA, 22 bp, synthetic construct
  source 1..22: mol_type = other DNA
aagcagatggccacagaactag

SEQ ID NO: 75 - DNA, 19 bp, synthetic construct
  source 1..19: mol_type = other DNA
tggttcccagtttttcatc

SEQ ID NO: 76 - DNA, 19 bp, synthetic construct
  source 1..19: mol_type = other DNA
cgagttgtccacagtcagc

SEQ ID NO: 77 - DNA, 24 bp, synthetic construct
  source 1..24: mol_type = other DNA
cggattatagtctctgaccatgtg

SEQ ID NO: 78 - DNA, 21 bp, synthetic construct
  source 1..21: mol_type = other DNA
tgtgtcaccatgttcttcagg

SEQ ID NO: 79 - DNA, 23 bp, synthetic construct
  source 1..23: mol_type = other DNA
ttgctcactagtggggtcgcagg

SEQ ID NO: 80 - DNA, 23 bp, synthetic construct
  source 1..23: mol_type = other DNA
cttcctccggccgagacgaaggg

SEQ ID NO: 81 - DNA, 24 bp, synthetic construct
  source 1..24: mol_type = other DNA
aaaagtttcctgatgtgaaagtca

SEQ ID NO: 82 - DNA, 20 bp, synthetic construct
  source 1..20: mol_type = other DNA
aaaaacagatgctccccaga

SEQ ID NO: 83 - DNA, 20 bp, synthetic construct
  source 1..20: mol_type = other DNA
cgtcacagagcaacaagaca

SEQ ID NO: 84 - DNA, 20 bp, synthetic construct
  source 1..20: mol_type = other DNA
actgagcacagaggaacacg